(19) United States
(12) Patent Application Publication
(10) Pub. No.: US 2025/0340660 A1
(43) Pub. Date: Nov. 6, 2025

(54) ANTI-CD30L ANTIBODIES, FORMULATIONS THEREFOR, AND USES THEREOF

(71) Applicants: PROMETHEUS BIOSCIENCES, INC., San Diego, CA (US); DR. FALK PHARMA GMBH, Freiburg im Breisgau (DE)

(72) Inventors: Johan Fransson, San Diego, CA (US); Brian Connolly, Berkeley, CA (US); Cindy T. Dickerson, Encinitas, CA (US); Jessie-Farah Fecteau, Escondido, CA (US); Mark Renshaw, San Diego, CA (US); Olivier Laurent, San Diego, CA (US); Burton Barnett, Poway, CA (US); Ernesto J. Munoz, Rancho Sante Fe, CA (US)

(73) Assignees: PROMETHEUS BIOSCIENCES, INC., San Diego, CA (US); DR. FALK PHARMA GMBH, Freiburg im Breisgau (DE)

(21) Appl. No.: 18/997,306

(22) PCT Filed: Jul. 24, 2023
(86) PCT No.: PCT/US2023/070856

§ 371 Date: Jan. 21, 2025

Related U.S. Application Data
(60) Provisional application No. 63/369,361, filed on Jul. 25, 2022.
(60) Provisional application No. 63/371,553, filed on Aug. 16, 2022.

(51) Int. Cl. C07K 16/28; A61K 9/08; A61K 39/00; A61K 47/18; A61K 47/22; A61K 47/26; A61P 1/00

(52) U.S. Cl. CPC C07K 16/2875; A61K 9/08; A61K 47/183; A61K 47/22; A61K 47/26; A61P 1/00; A61K 2039/505

(57) ABSTRACT

Described herein are anti-CD30L antibodies and pharmaceutical compositions for the treatment of autoimmune diseases and disorders such inflammatory bowel disease (IBD), including Crohn's Disease (CD) and ulcerative colitis (UC).

### CROSS-REFERENCE TO RELATED APPLICATIONS

This application claims benefit of U.S. Provisional Patent Application No. 63/369,361, filed on Jul. 25, 2022, and U.S. Provisional Patent Application No. 63/371,553, filed on Aug. 16, 2022, both of which are incorporated herein by reference in their entireties.

### REFERENCE TO A SEQUENCE LISTING

This application contains a Sequence Listing which has been submitted electronically in XML format and is hereby incorporated by reference in its entirety. Said XML copy, created on Jun. 26, 2023, is named 56884-409601.xml and is 663813 bytes in size.

### 1. BACKGROUND

Autoimmune disease occurs when the immune system attacks self-molecules as a result of a breakdown of immunologic tolerance to autoreactive immune cells. Many autoimmune disorders have been strongly associated with genetic, infectious, and/or environmental predisposing factors yielding multiple disorders and symptoms ranging from organ-specific dysfunction to systemic dysfunction. Inflammatory bowel disease (IBD) refers to a collection of intestinal disorders causing inflammatory conditions in the gastrointestinal tract. The primary types of IBD are ulcerative colitis (UC) and Crohn's Disease (CD). These diseases are prevalent, with about 1.86 million people diagnosed globally with UC, and about 1.3 million people diagnosed globally with CD. Severe forms of IBD may result in or be characterized by intestinal fibrosis, which is the accumulation of scar tissue in the intestinal wall. The pathogenesis of IBD is thought to involve an uncontrolled immune response that may be triggered by certain environmental factors in a genetically susceptible host. The heterogeneity of disease pathogenesis and clinical course, combined with the variable response to treatment and its associated side effects, suggests a targeted therapeutic approach to treating these diseases is a desirable treatment strategy. Yet there are very few targeted therapies available to IBD patients, especially those patients who may be non-responsive or lose response to existing IBD therapies.

### 2. SUMMARY

Provided herein are antibodies that bind CD30 ligand (also referred to as CD30L, CD153, TNFSF8) and are useful in the treatment of autoimmune disorders such as IBD. Generally, in an aspect, the antibodies described herein effectively inhibit an interaction between CD30L and CD30 in order to effectively reduce, inhibit, or prevent immune activation (for example, an inflammatory response). Furthermore, the anti-CD30L antibodies described herein also comprise properties useful for therapeutic application, including, for example, reduced and/or low immunogenicity. The antibodies described herein also inhibit expression/secretion of certain inflammatory cytokines (e.g., interleukin-6 and interleukin-8) that play a key role in inflammatory and autoimmune diseases such as inflammatory bowel disease, Crohn's disease, and ulcerative colitis.

Accordingly, in one aspect, provided herein is an antibody or antigen binding fragment thereof that binds CD30L, wherein the antibody or antigen binding fragment thereof comprises: (a) an immunoglobulin heavy chain CDR1 (CDR-H1) comprising the amino acid sequence set forth in any one of SEQ ID NOs: 100-139, 220-234, 465-489, 628-641, and 712-723; (b) an immunoglobulin heavy chain CDR2 (CDR-H2) comprising the amino acid sequence set forth in any one of SEQ ID NOs: 140-179, 235-249, 490-499, 513-527, 642-655, and 724-735; (c) an immunoglobulin heavy chain CDR3 (CDR-H3) comprising the amino acid sequence set forth in any one of SEQ Id NOs: 180-219, 250-264, 528-552, 656-669, and 736-743; (d) an immunoglobulin light chain CDR1 (CDR-L1) comprising the amino acid sequence set forth in any one of SEQ ID NOs: 300-339, 420-434, 553-577, 670-683, and 744-751; (d) an immunoglobulin light chain CDR2 (CDR-L2) comprising the amino acid sequence set forth in any one of SEQ ID NOs: 340-379, 435-449, 578-602, 684-697, and 752-759; and/or (e) an immunoglobulin light chain CDR3 (CDR-L3) comprising the amino acid sequence set forth in any one of SEQ ID NOs: 380-419, 450-464, 603-627, 698-711, and 760-765.

In some embodiments, the antibody or antigen binding fragment thereof comprises: (a) a CDR-H1 comprising the amino acid sequence set forth in any one of SEQ ID NOs: 100-109, 628, 635, and 712-723; (b) a CDR-H2 comprising the amino acid sequence set forth in any one of SEQ ID NOs: 140-149, 642, 649, and 724-735; (c) a CDR-H3 comprising the amino acid sequence set forth in any one of SEQ ID NOs: 180-189, 656, 663, and 736-743; (d) a CDR-L1 comprising the amino acid sequence set forth in any one of SEQ ID NOs: 300-309, 670, 677, and 744-751; (e) a CDR-L2 comprising the amino acid sequence set forth in any one of SEQ ID NOs: 340-349, 684, 691, and 752-759; and/or (f) a CDR-L3 comprising the amino acid sequence set forth in any one of SEQ ID Nos: 380-389, 698, 705, and 760-765.

In some embodiments, the antibody or antigen binding fragment thereof comprises: (a) a CDR-H1 comprising the amino acid sequence set forth in any one of SEQ ID NOs: 105-109, 628, 635, and 712-723; (b) a CDR-H2 comprising the amino acid sequence set forth in any one of SEQ ID NOs: 145-149, 642, 649, and 724-735; (c) a CDR-H3 comprising the amino acid sequence set forth in any one of SEQ ID NOs: 185-189, 656, 663, and 736-743; (d) a CDR-L1 comprising the amino acid sequence set forth in any one of SEQ ID NOs: 305-309, 670, 677, and 744-751; (e) a CDR-L2 comprising the amino acid sequence set forth in any one of SEQ ID NOs: 345-349, 684, 691, and 752-759; and/or (f) a CDR-L3 comprising the amino acid sequence set forth in any one of SEQ ID NOs: 385-389, 698, 705, and 760-765.

In one aspect, provided herein is an antibody or antigen binding fragment thereof that binds CD30L, wherein the antibody or antigen binding fragment thereof binds to an epitope comprising one or more amino acids in CD30L selected from the group consisting of N165, K166, I168, K169, and D234, wherein the amino acids are numbered according to the amino acid sequence of CD30L as set forth in SEQ ID NO:33. In some embodiments, the antibody or antigen binding fragment thereof binds to an epitope comprising: (i) any one amino acid in CD30L selected from the group consisting of N165, K166, I168, K169, and D234; (ii) any two amino acids in CD30L selected from the group consisting of N165, K166, I168, K169, and D234; (iii) any three amino acids in CD30L selected from the group consisting of N165, K166, I168, K169, and D234; (iv) any four amino acids in CD30L selected from the group consisting of N165, K166, I168, K169, and D234; or (v) any five amino acids in CD30L selected from the group consisting of N165, K166, I168, K169, and D234, wherein the amino acids are numbered according to the amino acid sequence of CD30L as set forth in SEQ ID NO: 33.

In one aspect, provided herein is an antibody or antigen binding fragment thereof that binds CD30L, wherein the antibody or antigen binding fragment thereof binds to an epitope comprising one or more amino acids in CD30L selected from the group consisting of H167, S217, and D118, wherein the amino acids are numbered according to the amino acid sequence of CD30L as set forth in SEQ ID NO:33. In some embodiments, the antibody or antigen binding fragment thereof binds to an epitope comprising: (i) any one amino acid in CD30L selected from the group consisting of H167, S217, and D118; (ii) any two amino acids in CD30L selected from the group consisting of H167, S217, and D118; or (iii) any three amino acids in CD30L selected from the group consisting of H167, S217, and D118, wherein the amino acids are numbered according to the amino acid sequence of CD30L as set forth in SEQ ID NO:33.

In one aspect, provided herein is an antibody or antigen binding fragment thereof that binds CD30L, wherein the antibody or antigen binding fragment thereof binds to an epitope comprising one or more amino acids in CD30L selected from the group consisting of D118, N165, K166, H167, I168, K169, S217, and D234, wherein the amino acids are numbered according to the amino acid sequence of CD30L as set forth in SEQ ID NO:33. In some embodiments, the antibody or antigen binding fragment thereof binds to an epitope comprising: (i) any one amino acid in CD30L selected from the group consisting of D118, N165, K166, H167, I168, K169, S217, and D234; (ii) any two amino acids in CD30L selected from the group consisting of D118, N165, K166, H167, I168, K169, S217, and D234; (iii) any three amino acids in CD30L selected from the group consisting of D118, N165, K166, H167, I168, K169, S217, and D234; (iv) any four amino acids in CD30L selected from the group consisting of D118, N165, K166, H167, I168, K169, S217, and D234; (v) any five amino acids in CD30L selected from the group consisting of D118, N165, K166, H167, I168, K169, S217, and D234; (vi) any six amino acids in CD30L selected from the group consisting of D118, N165, K166, H167, I168, K169, S217, and D234; (vii) any seven amino acids in CD30L selected from the group consisting of D118, N165, K166, H167, I168, K169, S217, and D234; or (viii) any five amino acids in CD30L selected from the group consisting of D118, N165, K166, H167, I168, K169, S217, and D234; wherein the amino acids are numbered according to the amino acid sequence of CD30L as set forth in SEQ ID NO:33.

In one aspect, provided herein is an anti-CD30L antibody or antigen binding fragment thereof, wherein the antibody or antigen binding fragment thereof binds to an epitope comprising K169 in CD30L, wherein the amino acids are numbered according to the amino acid sequence of CD30L as set forth in SEQ ID NO:33.

In some embodiments, the antibody or antigen binding fragment thereof binds to an epitope comprising D234 in CD30L, wherein the amino acids are numbered according to the amino acid sequence of CD30L as set forth in SEQ ID NO:33. In some embodiments, the epitope further comprises N165 in CD30L, wherein the amino acids are numbered according to the amino acid sequence of CD30L as set forth in SEQ ID NO:33. In some embodiments, the epitope further comprises I168 in CD30L, wherein the amino acids are numbered according to the amino acid sequence of CD30L as set forth in SEQ ID NO:33. In some embodiments, the epitope further comprises K166 in CD30L, wherein the amino acids are numbered according to the amino acid sequence of CD30L as set forth in SEQ ID NO:33. In some embodiments, the epitope further comprises H167 in CD30L, wherein the amino acids are numbered according to the amino acid sequence of CD30L as set forth in SEQ ID NO:33. In some embodiments, the epitope further comprises S217 in CD30L, wherein the amino acids are numbered according to the amino acid sequence of CD30L as set forth in SEQ ID NO: 33. In some embodiments, the epitope further comprises D118 in CD30L, wherein the amino acids are numbered according to the amino acid sequence of CD30L as set forth in SEQ ID NO:33.

In some embodiments, the antibody or antigen binding fragment thereof comprises: (a) a CDR-H1 comprising the amino acid sequence set forth in any one of SEQ ID NOs: 110-119, 629, 636, and 712-723; (b) a CDR-H2 comprising the amino acid sequence set forth in any one of SEQ ID NOs: 150-159, 643, 650, and 724-735; (c) a CDR-H3 comprising the amino acid sequence set forth in any one of SEQ ID NOs: 190-199, 657, 664, and 736-743; (d) a CDR-L1 comprising the amino acid sequence set forth in any one of SEQ ID NOs: 310-319, 671, 678, and 744-751; (e) a CDR-L2 comprising the amino acid sequence set forth in any one of SEQ ID NOs: 350-359, 685, 692, and 752-759; and/or (f) a CDR-L3 comprising the amino acid sequence set forth in any one of SEQ ID NOs: 390-399, 699, 706, and 760-765.

In some embodiments, the antibody or antigen binding fragment thereof comprises: (a) a CDR-H1 comprising the amino acid sequence set forth in any one of SEQ ID NOs: 115-119, 629, 636, and 712-723; (b) a CDR-H2 comprising the amino acid sequence set forth in any one of SEQ ID NOs: 155-159, 643, 650, and 724-735; (c) a CDR-H3 comprising the amino acid sequence set forth in any one of SEQ ID NOs: 195-199, 657, 664, and 736-743; (d) a CDR-L1 comprising the amino acid sequence set forth in any one of SEQ ID NOs: 315-319, 671, 678, and 744-751; (e) a CDR-L2 comprising the amino acid sequence set forth in any one of SEQ ID NOs: 355-359, 685, 692, and 752-759; and/or (f) a CDR-L3 comprising the amino acid sequence set forth in any one of SEQ ID NOs: 395-399, 699, 706, and 760-765.

In some embodiments, the antibody or antigen binding fragment thereof comprises: (a) a CDR-H1 comprising the amino acid sequence set forth in any one of SEQ ID NOs: 120-129, and 712-723; (b) a CDR-H2 comprising the amino acid sequence set forth in any one of SEQ ID NOs: 160-169, and 724-735; (c) a CDR-H3 comprising the amino acid sequence set forth in any one of SEQ ID NOs: 200-209, and 736-743; (d) a CDR-L1 comprising the amino acid sequence set forth in any one of SEQ ID NOs: 320-329, and 744-751; (e) a CDR-L2 comprising the amino acid sequence set forth in any one of SEQ ID NOs: 360-369, and 752-759; and/or (f) a CDR-L3 comprising the amino acid sequence set forth in any one of SEQ ID NOs: 400-409, and 760-765.

In some embodiments, the antibody or antigen binding fragment thereof comprises: (a) a CDR-H1 comprising the amino acid sequence set forth in any one of SEQ ID NOs: 125-129, and 712-723; (b) a CDR-H2 comprising the amino acid sequence set forth in any one of SEQ ID NOs: 165-169, and 724-735; (c) a CDR-H3 comprising the amino acid sequence set forth in any one of SEQ ID NOs: 205-209, and 736-743; (d) a CDR-L1 comprising the amino acid sequence set forth in any one of SEQ ID NOs: 325-329, and 744-751; (e) a CDR-L2 comprising the amino acid sequence set forth in any one of SEQ ID NOs: 365-369, and 752-759; and/or (f) a CDR-L3 comprising the amino acid sequence set forth in any one of SEQ ID NOs: 405-409, and 760-765.

In some embodiments, the antibody or antigen binding fragment thereof comprises: (a) a CDR-H1 comprising the amino acid sequence set forth in any one of SEQ ID NOs: 130-139, and 712-723; (b) a CDR-H2 comprising the amino acid sequence set forth in any one of SEQ ID NOs: 170-179, and 724-735; (c) a CDR-H3 comprising the amino acid sequence set forth in any one of SEQ ID NOs: 210-219, and 736-743; (d) a CDR-L1 comprising the amino acid sequence set forth in any one of SEQ ID NOs: 330-339, and 744-751; (e) a CDR-L2 comprising the amino acid sequence set forth in any one of SEQ ID NOs: 370-379, and 752-759; and/or (f) a CDR-L3 comprising the amino acid sequence set forth in any one of SEQ ID NOs: 410-419, and 760-765.

In some embodiments, the antibody or antigen binding fragment thereof comprises: (a) a CDR-H1 comprising the amino acid sequence set forth in any one of SEQ ID NOs: 130-134; (b) a CDR-H2 comprising the amino acid sequence set forth in any one of SEQ ID NOs: 170-174; (c) a CDR-H3 comprising the amino acid sequence set forth in any one of SEQ ID NOs: 210-214; (d) a CDR-L1 comprising the amino acid sequence set forth in any one of SEQ ID NOs: 330-334; (e) a CDR-L2 comprising the amino acid sequence set forth in any one of SEQ ID NOs: 370-374; and/or (f) a CDR-L3 comprising the amino acid sequence set forth in any one of SEQ ID NOs: 410-414.

In some embodiments, the antibody or antigen binding fragment thereof comprises: (a) a CDR-H1 comprising the amino acid sequence set forth in any one of SEQ ID NOs: 135-139, and 712-723; (b) a CDR-H2 comprising the amino acid sequence set forth in any one of SEQ ID NOs: 175-179, and 724-735; (c) a CDR-H3 comprising the amino acid sequence set forth in any one of SEQ ID NOs: 215-219, and 736-743; (d) a CDR-L1 comprising the amino acid sequence set forth in any one of SEQ ID NOs: 335-339, and 744-751; (e) a CDR-L2 comprising the amino acid sequence set forth in any one of SEQ ID NOs: 375-379, and 752-759; and/or (f) a CDR-L3 comprising the amino acid sequence set forth in any one of SEQ ID NOs: 415-419, and 760-765.

In some embodiments, the antibody or antigen binding fragment thereof comprises: (a) a CDR-H1 comprising the amino acid sequence set forth in any one of SEQ ID NOs: 220-224, and 712-723; (b) a CDR-H2 comprising the amino acid sequence set forth in any one of SEQ ID NOs: 235-239, and 724-735; (c) a CDR-H3 comprising the amino acid sequence set forth in any one of SEQ ID NOs: 250-254, and 736-743; (d) a CDR-L1 comprising the amino acid sequence set forth in any one of SEQ ID NOs: 420-424, and 744-751; (e) a CDR-L2 comprising the amino acid sequence set forth in any one of SEQ ID NOs: 435-439, and 752-759; and/or (f) a CDR-L3 comprising the amino acid sequence set forth in any one of SEQ ID NOs: 450-454, and 760-765.

In some embodiments, the antibody or antigen binding fragment thereof comprises an immunoglobulin variable region heavy chain and an immunoglobulin variable region light chain, wherein: (a) the immunoglobulin variable region heavy chain (VH) comprises an amino acid sequence having at least about 90, 95, 97, 98, 99, or 100% sequence identity to SEQ ID NO: 17; and/or (b) the immunoglobulin variable region light chain (VL) comprises an amino acid sequence having at least about 90, 95, 97, 98, 99, or 100% sequence identity to SEQ ID NO: 18.

In some embodiments, the antibody or antigen binding fragment thereof comprises: (a) a CDR-H1 comprising the amino acid sequence set forth in any one of SEQ ID NOs: 225-229, and 712-723; (b) a CDR-H2 comprising the amino acid sequence set forth in any one of SEQ ID NOs: 240-244, and 724-735; (c) a CDR-H3 comprising the amino acid sequence set forth in any one of SEQ ID NOs: 255-259, and 736-743; (d) a CDR-L1 comprising the amino acid sequence set forth in any one of SEQ ID NOs: 425-429, and 744-751; (e) a CDR-L2 comprising the amino acid sequence set forth in any one of SEQ ID NOs: 440-444, and 752-759; and/or (f) a CDR-L3 comprising the amino acid sequence set forth in any one of SEQ ID NOs: 455-459, and 760-765.

In some embodiments, the antibody or antigen binding fragment thereof comprises an immunoglobulin variable region heavy chain and an immunoglobulin variable region light chain, wherein: (a) VH comprises an amino acid sequence having at least about 90, 95, 97, 98, 99, or 100% sequence identity to SEQ ID NO: 19; and/or (b) VL comprises an amino acid sequence having at least about 90, 95, 97, 98, 99, or 100% sequence identity to SEQ ID NO: 20.

In some embodiments, the antibody or antigen binding fragment thereof comprises: (a) a CDR-H1 comprising the amino acid sequence set forth in any one of SEQ ID NOs: 230-234, and 712-723; (b) a CDR-H2 comprising the amino acid sequence set forth in any one of SEQ ID NOs: 245-249, and 724-735; (c) a CDR-H3 comprising the amino acid sequence set forth in any one of SEQ ID NOs: 260-264, and 736-743; (d) a CDR-L1 comprising the amino acid sequence set forth in any one of SEQ ID NOs: 430-434, and 744-751; (e) a CDR-L2 comprising the amino acid sequence set forth in any one of SEQ ID NOs: 445-449, and 752-759; and/or (f) a CDR-L3 comprising the amino acid sequence set forth in any one of SEQ ID NOs: 460-464, and 760-765.

In some embodiments, the antibody or antigen binding fragment thereof comprises an immunoglobulin variable region heavy chain and an immunoglobulin variable region light chain, wherein: (a) VH comprises an amino acid sequence having at least about 90, 95, 97, 98, 99, or 100% sequence identity to SEQ ID NO: 21; and/or (b) VL comprises an amino acid sequence having at least about 90, 95, 97, 98, 99, or 100% sequence identity to SEQ ID NO: 22.

In some embodiments, the antibody or antigen binding fragment thereof comprises: (a) a CDR-H1 comprising the amino acid sequence set forth in any one of SEQ ID NOs: 465-469, 631, 638, and 712-723; (b) a CDR-H2 comprising the amino acid sequence set forth in any one of SEQ ID NOs: 490-494, 645, 652, and 724-735; (c) a CDR-H3 comprising the amino acid sequence set forth in any one of SEQ ID NOs: 528-532, 659, 666, and 736-743; (d) a CDR-L1 comprising the amino acid sequence set forth in any one of SEQ ID NOs: 553-557, 673, 680, and 744-751; (e) a CDR-L2 comprising the amino acid sequence set forth in any one of SEQ ID NOs: 578-582, 687, 694, and 752-759; and/or (f) a CDR-L3 comprising the amino acid sequence set forth in any one of SEQ ID NOs: 603-607, 701, 708, and 760-765. In some embodiments, the antibody or antigen binding fragment thereof comprises: (a) a CDR-H1 comprising the amino acid sequence set forth in any one of SEQ ID NOs: 470-474, 632, 639, and 712-723; (b) a CDR-H2 comprising the amino acid sequence set forth in any one of SEQ ID NOs: 495-499, 646, 653, and 724-735; (c) a CDR-H3 comprising the amino acid sequence set forth in any one of SEQ ID NOs: 533-537, 660, 667, and 736-743; (d) a CDR-L1 comprising the amino acid sequence set forth in any one of SEQ ID NOs: 558-562, 674, 681, and 744-751; (e) a CDR-L2 comprising the amino acid sequence set forth in any one of SEQ ID NOs: 583-587, 688, 695, and 752-759; and/or (f) a CDR-L3 comprising the amino acid sequence set forth in any one of SEQ ID NOs: 608-612, 702, 709, and 760-765. In some embodiments, the antibody or antigen binding fragment thereof comprises: (a) a CDR-H1 comprising the amino acid sequence set forth in any one of SEQ ID NOs: 475-479, 633, 640, and 712-723; (b) a CDR-H2 comprising the amino acid sequence set forth in any one of SEQ ID NOs: 513-517, 647, 654, and 724-735; (c) a CDR-H3 comprising the amino acid sequence set forth in any one of SEQ ID NOs: 538-542, 661, 668, and 736-743; (d) a CDR-L1 comprising the amino acid sequence set forth in any one of SEQ ID NOs: 563-567, 675, 682, and 744-751; (e) a CDR-L2 comprising the amino acid sequence set forth in any one of SEQ ID NOs: 588-592, 689, 696, and 752-759; and/or (f) a CDR-L3 comprising the amino acid sequence set forth in any one of SEQ ID NOs: 613-617, 703, 710, and 760-765. In some embodiments, the antibody or antigen binding fragment thereof comprises: (a) a CDR-H1 comprising the amino acid sequence set forth in any one of SEQ ID NOs: 480-484, 630, 637, and 712-723; (b) a CDR-H2 comprising the amino acid sequence set forth in any one of SEQ ID NOs: 518-522, 644, 651, and 724-735; (c) a CDR-H3 comprising the amino acid sequence set forth in any one of SEQ ID NOs: 543-547, 658, 665, and 736-743; (d) a CDR-L1 comprising the amino acid sequence set forth in any one of SEQ ID NOs: 568-572, 672, 679, and 744-751; (e) a CDR-L2 comprising the amino acid sequence set forth in any one of SEQ ID NOs: 593-597, 686, 693, and 752-759; and/or (f) a CDR-L3 comprising the amino acid sequence set forth in any one of SEQ ID NOs: 618-622, 700, 707, and 760-765. In some embodiments, the antibody or antigen binding fragment thereof comprises: (a) a CDR-H1 comprising the amino acid sequence set forth in any one of SEQ ID NOs: 485-489, 634, 641, and 712-723; (b) a CDR-H2 comprising the amino acid sequence set forth in any one of SEQ ID NOs: 523-527, 648, 655, and 724-735; (c) a CDR-H3 comprising the amino acid sequence set forth in any one of SEQ ID NOs: 548-552, 662, 669, and 736-743; (d) a CDR-L1 comprising the amino acid sequence set forth in any one of SEQ ID NOs: 573-577, 676, 683, and 744-751; (e) a CDR-L2 comprising the amino acid sequence set forth in any one of SEQ ID NOs: 598-602, 690, 697, and 752-759; and/or (f) a CDR-L3 comprising the amino acid sequence set forth in any one of SEQ ID NOs: 623-627, 704, 711, and 760-765.

In some embodiments, the antibody or antigen binding fragment thereof comprises: (a) a CDR-H1 comprising the amino acid sequence set forth in any one of SEQ ID NOs: 712-723; (b) a CDR-H2 comprising the amino acid sequence set forth in any one of SEQ ID NOs: 724-735; (c) a CDR-H3 comprising the amino acid sequence set forth in any one of SEQ ID NOs: 736-743; (d) a CDR-L1 comprising the amino acid sequence set forth in any one of SEQ ID NOs: 744-751; (e) a CDR-L2 comprising the amino acid sequence set forth in any one of SEQ ID NOs: 752-759; and/or (f) a CDR-L3 comprising the amino acid sequence set forth in any one of SEQ ID NOs: 760-765. In some embodiments, the antibody or antigen binding fragment thereof comprises: (a) a CDR-H1 comprising the amino acid sequence set forth in any one of SEQ ID NOs: 712, 714, 716, 718, 720, and 722; (b) a CDR-H2 comprising the amino acid sequence set forth in any one of SEQ ID NOs: 724, 726, 728, 730, 732, and 734; (c) a CDR-H3 comprising the amino acid sequence set forth in any one of SEQ ID NOs: 736, 738, 740, and 742; (d) a CDR-L1 comprising the amino acid sequence set forth in any one of SEQ ID NOs: 744, 746, 748, and 750; (e) a CDR-L2 comprising the amino acid sequence set forth in any one of SEQ ID NOs: 752, 754, 756, and 758; and/or (f) a CDR-L3 comprising the amino acid sequence set forth in any one of SEQ ID NOs: 760, 762, and 764. In some embodiments, the antibody or antigen binding fragment thereof comprises: (a) a CDR-H1 comprising the amino acid sequence set forth in any one of SEQ ID NOs: 713, 715, 717, 719, 721, and 723; (b) a CDR-H2 comprising the amino acid sequence set forth in any one of SEQ ID NOs: 725, 727, 729, 731, 733, and 735; (c) a CDR-H3 comprising the amino acid sequence set forth in any one of SEQ ID NOs: 737, 739, 741, and 743; (d) a CDR-L1 comprising the amino acid sequence set forth in any one of SEQ ID NOs: 745, 747, 749, and 751; (e) a CDR-L2 comprising the amino acid sequence set forth in any one of SEQ ID NOs: 753, 755, 757, and 759; and/or (f) a CDR-L3 comprising the amino acid sequence set forth in any one of SEQ ID NOs: 761, 763, and 765. In some embodiments, the antibody or antigen binding fragment thereof comprises: (a) a CDR-H1 comprising the amino acid sequence set forth in SEQ ID NO: 712; (b) a CDR-H2 comprising the amino acid sequence set forth in SEQ ID NO: 730; (c) a CDR-H3 comprising the amino acid sequence set forth in SEQ ID NO: 736; (d) a CDR-L1 comprising the amino acid sequence set forth in SEQ ID NO: 744; (e) a CDR-L2 comprising the amino acid sequence set forth SEQ ID NO: 752; and/or (f) a CDR-L3 comprising the amino acid sequence set forth SEQ ID NO: 760. In some embodiments, the antibody or antigen binding fragment thereof comprises: (v)(a) a CDR-H1 comprising the amino acid sequence set forth in SEQ ID NO: 713; (b) a CDR-H2 comprising the amino acid sequence set forth in SEQ ID NO: 731; (c) a CDR-H3 comprising the amino acid sequence set forth in SEQ ID NO: 737; (d) a CDR-L1 comprising the amino acid sequence set forth in SEQ ID NO: 745; (e) a CDR-L2 comprising the amino acid sequence set forth SEQ ID NO: 753; and/or (f) a CDR-L3 comprising the amino acid sequence set forth SEQ ID NO: 761. In some embodiments, the antibody or antigen binding fragment thereof comprises: (a) a CDR-H1 comprising the amino acid sequence set forth in SEQ ID NO: 712; (b) a CDR-H2 comprising the amino acid sequence set forth in SEQ ID NO: 724; (c) a CDR-H3 comprising the amino acid sequence set forth in SEQ ID NO: 736; (d) a CDR-L1 comprising the amino acid sequence set forth in SEQ ID NO: 744; (e) a CDR-L2 comprising the amino acid sequence set forth SEQ ID NO: 752; and/or (f) a CDR-L3 comprising the amino acid sequence set forth SEQ ID NO: 760. In some embodiments, the antibody or antigen binding fragment thereof comprises: (a) a CDR-H1 comprising the amino acid sequence set forth in SEQ ID NO: 713; (b) a CDR-H2 comprising the amino acid sequence set forth in SEQ ID NO: 725; (c) a CDR-H3 comprising the amino acid sequence set forth in SEQ ID NO: 737; (d) a CDR-L1 comprising the amino acid sequence set forth in SEQ ID NO: 745; (e) a CDR-L2 comprising the amino acid sequence set forth SEQ ID NO: 753; and/or (f) a CDR-L3 comprising the amino acid sequence set forth SEQ ID NO: 761. In some embodiments, the antibody or antigen binding fragment thereof comprises: (a) a CDR-H1 comprising the amino acid sequence set forth in SEQ ID NO: 714; (b) a CDR-H2 comprising the amino acid sequence set forth in SEQ ID NO: 726; (c) a CDR-H3 comprising the amino acid sequence set forth in SEQ ID NO: 736; (d) a CDR-L1 comprising the amino acid sequence set forth in SEQ ID NO: 744; (e) a CDR-L2 comprising the amino acid sequence set forth SEQ ID NO: 752; and/or (f) a CDR-L3 comprising the amino acid sequence set forth SEQ ID NO: 760. In some embodiments, the antibody or antigen binding fragment thereof comprises: (a) a CDR-H1 comprising the amino acid sequence set forth in SEQ ID NO: 715; (b) a CDR-H2 comprising the amino acid sequence set forth in SEQ ID NO: 727; (c) a CDR-H3 comprising the amino acid sequence set forth in SEQ ID NO: 737; (d) a CDR-L1 comprising the amino acid sequence set forth in SEQ ID NO: 745; (e) a CDR-L2 comprising the amino acid sequence set forth SEQ ID NO: 753; and/or (f) a CDR-L3 comprising the amino acid sequence set forth SEQ ID NO: 761. In some embodiments, the antibody or antigen binding fragment thereof comprises: (a) a CDR-H1 comprising the amino acid sequence set forth in SEQ ID NO: 716; (b) a CDR-H2 comprising the amino acid sequence set forth in SEQ ID NO: 728; (c) a CDR-H3 comprising the amino acid sequence set forth in SEQ ID NO: 736; (d) a CDR-L1 comprising the amino acid sequence set forth in SEQ ID NO: 744; (e) a CDR-L2 comprising the amino acid sequence set forth SEQ ID NO: 752; and/or (f) a CDR-L3 comprising the amino acid sequence set forth SEQ ID NO: 760. In some embodiments, the antibody or antigen binding fragment thereof comprises: (a) a CDR-H1 comprising the amino acid sequence set forth in SEQ ID NO: 717; (b) a CDR-H2 comprising the amino acid sequence set forth in SEQ ID NO: 729; (c) a CDR-H3 comprising the amino acid sequence set forth in SEQ ID NO: 737; (d) a CDR-L1 comprising the amino acid sequence set forth in SEQ ID NO: 745; (e) a CDR-L2 comprising the amino acid sequence set forth SEQ ID NO: 753; and/or (f) a CDR-L3 comprising the amino acid sequence set forth SEQ ID NO: 761. In some embodiments, the antibody or antigen binding fragment thereof comprises: (a) a CDR-H1 comprising the amino acid sequence set forth in SEQ ID NO: 718; (b) a CDR-H2 comprising the amino acid sequence set forth in SEQ ID NO: 730; (c) a CDR-H3 comprising the amino acid sequence set forth in SEQ ID NO: 738; (d) a CDR-L1 comprising the amino acid sequence set forth in SEQ ID NO: 746; (e) a CDR-L2 comprising the amino acid sequence set forth SEQ ID NO: 754; and/or (f) a CDR-L3 comprising the amino acid sequence set forth SEQ ID NO: 762. In some embodiments, the antibody or antigen binding fragment thereof comprises: (a) a CDR-H1 comprising the amino acid sequence set forth in SEQ ID NO: 719; (b) a CDR-H2 comprising the amino acid sequence set forth in SEQ ID NO: 731; (c) a CDR-H3 comprising the amino acid sequence set forth in SEQ ID NO: 739; (d) a CDR-L1 comprising the amino acid sequence set forth in SEQ ID NO: 747; (e) a CDR-L2 comprising the amino acid sequence set forth SEQ ID NO: 755; and/or (f) a CDR-L3 comprising the amino acid sequence set forth SEQ ID NO: 763. In some embodiments, the antibody or antigen binding fragment thereof comprises: (a) a CDR-H1 comprising the amino acid sequence set forth in SEQ ID NO: 720; (b) a CDR-H2 comprising the amino acid sequence set forth in SEQ ID NO: 732; (c) a CDR-H3 comprising the amino acid sequence set forth in SEQ ID NO: 740; (d) a CDR-L1 comprising the amino acid sequence set forth in SEQ ID NO: 748; (e) a CDR-L2 comprising the amino acid sequence set forth SEQ ID NO: 756; and/or (f) a CDR-L3 comprising the amino acid sequence set forth SEQ ID NO: 760. In some embodiments, the antibody or antigen binding fragment thereof comprises: (a) a CDR-H1 comprising the amino acid sequence set forth in SEQ ID NO: 721; (b) a CDR-H2 comprising the amino acid sequence set forth in SEQ ID NO: 733; (c) a CDR-H3 comprising the amino acid sequence set forth in SEQ ID NO: 741; (d) a CDR-L1 comprising the amino acid sequence set forth in SEQ ID NO: 749; (e) a CDR-L2 comprising the amino acid sequence set forth SEQ ID NO: 757; and/or (f) a CDR-L3 comprising the amino acid sequence set forth SEQ ID NO: 761. In some embodiments, the antibody or antigen binding fragment thereof comprises: (a) a CDR-H1 comprising the amino acid sequence set forth in SEQ ID NO: 722; (b) a CDR-H2 comprising the amino acid sequence set forth in SEQ ID NO: 734; (c) a CDR-H3 comprising the amino acid sequence set forth in SEQ ID NO: 742; (d) a CDR-L1 comprising the amino acid sequence set forth in SEQ ID NO: 750; (e) a CDR-L2 comprising the amino acid sequence set forth SEQ ID NO: 758; and/or (f) a CDR-L3 comprising the amino acid sequence set forth SEQ ID NO: 764. In some embodiments, the antibody or antigen binding fragment thereof comprises: (a) a CDR-H1 comprising the amino acid sequence set forth in SEQ ID NO: 723; (b) a CDR-H2 comprising the amino acid sequence set forth in SEQ ID NO: 735; (c) a CDR-H3 comprising the amino acid sequence set forth in SEQ ID NO: 743; (d) a CDR-L1 comprising the amino acid sequence set forth in SEQ ID NO: 751; (e) a CDR-L2 comprising the amino acid sequence set forth SEQ ID NO: 759; and/or (f) a CDR-L3 comprising the amino acid sequence set forth SEQ ID NO: 765.

In some embodiments, the antibody or antigen binding fragment thereof comprises: (a) a CDR-H1 comprising the amino acid sequence set forth in SEQ ID NO: 635; (b) a CDR-H2 comprising the amino acid sequence set forth in SEQ ID NO: 649; (c) a CDR-H3 comprising the amino acid sequence set forth in SEQ ID NO: 663; (d) a CDR-L1 comprising the amino acid sequence set forth in SEQ ID NO: 677; (e) a CDR-L2 comprising the amino acid sequence set forth SEQ ID NO: 691; and/or (f) a CDR-L3 comprising the amino acid sequence set forth SEQ ID NO: 705. In some embodiments, the antibody or antigen binding fragment thereof comprises: (a) a CDR-H1 comprising the amino acid sequence set forth in SEQ ID NO: 107; (b) a CDR-H2 comprising the amino acid sequence set forth in SEQ ID NO: 147; (c) a CDR-H3 comprising the amino acid sequence set forth in SEQ ID NO: 187; (d) a CDR-L1 comprising the amino acid sequence set forth in SEQ ID NO: 307; (e) a CDR-L2 comprising the amino acid sequence set forth SEQ ID NO: 347; and/or (f) a CDR-L3 comprising the amino acid sequence set forth SEQ ID NO: 387. In some embodiments, the antibody or antigen binding fragment thereof comprises: (a) a CDR-H1 comprising the amino acid sequence set forth in SEQ ID NO: 105; (b) a CDR-H2 comprising the amino acid sequence set forth in SEQ ID NO: 145; (c) a CDR-H3 comprising the amino acid sequence set forth in SEQ ID NO: 185; (d) a CDR-L1 comprising the amino acid sequence set forth in SEQ ID NO: 305; (e) a CDR-L2 comprising the amino acid sequence set forth SEQ ID NO: 345; and/or (f) a CDR-L3 comprising the amino acid sequence set forth SEQ ID NO: 385. In some embodiments, the antibody or antigen binding fragment thereof comprises: (a) a CDR-H1 comprising the amino acid sequence set forth in SEQ ID NO: 106; (b) a CDR-H2 comprising the amino acid sequence set forth in SEQ ID NO: 146; (c) a CDR-H3 comprising the amino acid sequence set forth in SEQ ID NO: 186; (d) a CDR-L1 comprising the amino acid sequence set forth in SEQ ID NO: 306; (e) a CDR-L2 comprising the amino acid sequence set forth SEQ ID NO: 346; and/or (f) a CDR-L3 comprising the amino acid sequence set forth SEQ ID NO: 386. In some embodiments, the antibody or antigen binding fragment thereof comprises: (a) a CDR-H1 comprising the amino acid sequence set forth in SEQ ID NO: 108; (b) a CDR-H2 comprising the amino acid sequence set forth in SEQ ID NO: 148; (c) a CDR-H3 comprising the amino acid sequence set forth in SEQ ID NO: 188; (d) a CDR-L1 comprising the amino acid sequence set forth in SEQ ID NO: 308; (e) a CDR-L2 comprising the amino acid sequence set forth SEQ ID NO: 348; and/or (f) a CDR-L3 comprising the amino acid sequence set forth SEQ ID NO: 388. In some embodiments, the antibody or antigen binding fragment thereof comprises: (a) a CDR-H1 comprising the amino acid sequence set forth in SEQ ID NO: 109; (b) a CDR-H2 comprising the amino acid sequence set forth in SEQ ID NO: 149; (c) a CDR-H3 comprising the amino acid sequence set forth in SEQ ID NO: 189; (d) a CDR-L1 comprising the amino acid sequence set forth in SEQ ID NO: 309; (e) a CDR-L2 comprising the amino acid sequence set forth SEQ ID NO: 349; and/or (f) a CDR-L3 comprising the amino acid sequence set forth SEQ ID NO: 389. In some embodiments, the antibody or antigen binding fragment thereof comprises: (a) a CDR-H1 comprising the amino acid sequence set forth in SEQ ID NO: 628; (b) a CDR-H2 comprising the amino acid sequence set forth in SEQ ID NO: 642; (c) a CDR-H3 comprising the amino acid sequence set forth in SEQ ID NO: 656; (d) a CDR-L1 comprising the amino acid sequence set forth in SEQ ID NO: 670; (e) a CDR-L2 comprising the amino acid sequence set forth SEQ ID NO: 684; and/or (f) a CDR-L3 comprising the amino acid sequence set forth SEQ ID NO: 698.

In some embodiments, the antibody or antigen binding fragment thereof comprises: (a) a CDR-H1 comprising the amino acid sequence set forth in SEQ ID NO: 636; (b) a CDR-H2 comprising the amino acid sequence set forth in SEQ ID NO: 650; (c) a CDR-H3 comprising the amino acid sequence set forth in SEQ ID NO: 664; (d) a CDR-L1 comprising the amino acid sequence set forth in SEQ ID NO: 678; (e) a CDR-L2 comprising the amino acid sequence set forth SEQ ID NO: 692; and/or (f) a CDR-L3 comprising the amino acid sequence set forth SEQ ID NO: 706. In some embodiments, the antibody or antigen binding fragment thereof comprises: (a) a CDR-H1 comprising the amino acid sequence set forth in SEQ ID NO: 117; (b) a CDR-H2 comprising the amino acid sequence set forth in SEQ ID NO: 157; (c) a CDR-H3 comprising the amino acid sequence set forth in SEQ ID NO: 197; (d) a CDR-L1 comprising the amino acid sequence set forth in SEQ ID NO: 317; (e) a CDR-L2 comprising the amino acid sequence set forth SEQ ID NO: 357; and/or (f) a CDR-L3 comprising the amino acid sequence set forth SEQ ID NO: 397. In some embodiments, the antibody or antigen binding fragment thereof comprises: (a) a CDR-H1 comprising the amino acid sequence set forth in SEQ ID NO: 115; (b) a CDR-H2 comprising the amino acid sequence set forth in SEQ ID NO: 155; (c) a CDR-H3 comprising the amino acid sequence set forth in SEQ ID NO: 195; (d) a CDR-L1 comprising the amino acid sequence set forth in SEQ ID NO: 315; (e) a CDR-L2 comprising the amino acid sequence set forth SEQ ID NO: 355; and/or (f) a CDR-L3 comprising the amino acid sequence set forth SEQ ID NO: 395. In some embodiments, the antibody or antigen binding fragment thereof comprises: (a) a CDR-H1 comprising the amino acid sequence set forth in SEQ ID NO: 116; (b) a CDR-H2 comprising the amino acid sequence set forth in SEQ ID NO: 156; (c) a CDR-H3 comprising the amino acid sequence set forth in SEQ ID NO: 196; (d) a CDR-L1 comprising the amino acid sequence set forth in SEQ ID NO: 316; (e) a CDR-L2 comprising the amino acid sequence set forth SEQ ID NO: 356; and/or (f) a CDR-L3 comprising the amino acid sequence set forth SEQ ID NO: 396. In some embodiments, the antibody or antigen binding fragment thereof comprises: (a) a CDR-H1 comprising the amino acid sequence set forth in SEQ ID NO: 118; (b) a CDR-H2 comprising the amino acid sequence set forth in SEQ ID NO: 158; (c) a CDR-H3 comprising the amino acid sequence set forth in SEQ ID NO: 198; (d) a CDR-L1 comprising the amino acid sequence set forth in SEQ ID NO: 318; (e) a CDR-L2 comprising the amino acid sequence set forth SEQ ID NO: 358; and/or (f) a CDR-L3 comprising the amino acid sequence set forth SEQ ID NO: 398. In some embodiments, the antibody or antigen binding fragment thereof comprises: (a) a CDR-H1 comprising the amino acid sequence set forth in SEQ ID NO: 119; (b) a CDR-H2 comprising the amino acid sequence set forth in SEQ ID NO: 159; (c) a CDR-H3 comprising the amino acid sequence set forth in SEQ ID NO: 199; (d) a CDR-L1 comprising the amino acid sequence set forth in SEQ ID NO: 319; (e) a CDR-L2 comprising the amino acid sequence set forth SEQ ID NO: 359; and/or (f) a CDR-L3 comprising the amino acid sequence set forth SEQ ID NO: 399. In some embodiments, the antibody or antigen binding fragment thereof comprises: (a) a CDR-H1 comprising the amino acid sequence set forth in SEQ ID NO: 629; (b) a CDR-H2 comprising the amino acid sequence set forth in SEQ ID NO: 643; (c) a CDR-H3 comprising the amino acid sequence set forth in SEQ ID NO: 657; (d) a CDR-L1 comprising the amino acid sequence set forth in SEQ ID NO: 671; (e) a CDR-L2 comprising the amino acid sequence set forth SEQ ID NO: 685; and/or (f) a CDR-L3 comprising the amino acid sequence set forth SEQ ID NO: 699.

In some embodiments, the antibody or antigen binding fragment thereof comprises: (a) a CDR-H1 comprising the amino acid sequence set forth in SEQ ID NO: 637; (b) a CDR-H2 comprising the amino acid sequence set forth in SEQ ID NO: 651; (c) a CDR-H3 comprising the amino acid sequence set forth in SEQ ID NO: 665; (d) a CDR-L1 comprising the amino acid sequence set forth in SEQ ID NO: 679; (e) a CDR-L2 comprising the amino acid sequence set forth SEQ ID NO: 693; and/or (f) a CDR-L3 comprising the amino acid sequence set forth SEQ ID NO: 707. In some embodiments, the antibody or antigen binding fragment thereof comprises: (a) a CDR-H1 comprising the amino acid sequence set forth in SEQ ID NO: 482; (b) a CDR-H2 comprising the amino acid sequence set forth in SEQ ID NO: 520; (c) a CDR-H3 comprising the amino acid sequence set forth in SEQ ID NO: 545; (d) a CDR-L1 comprising the amino acid sequence set forth in SEQ ID NO: 570; (e) a CDR-L2 comprising the amino acid sequence set forth SEQ ID NO: 595; and/or (f) a CDR-L3 comprising the amino acid sequence set forth SEQ ID NO: 620. In some embodiments, the antibody or antigen binding fragment thereof comprises: (a) a CDR-H1 comprising the amino acid sequence set forth in SEQ ID NO: 480; (b) a CDR-H2 comprising the amino acid sequence set forth in SEQ ID NO: 518; (c) a CDR-H3 comprising the amino acid sequence set forth in SEQ ID NO: 543; (d) a CDR-L1 comprising the amino acid sequence set forth in SEQ ID NO: 568; (e) a CDR-L2 comprising the amino acid sequence set forth SEQ ID NO: 593; and/or (f) a CDR-L3 comprising the amino acid sequence set forth SEQ ID NO: 618. In some embodiments, the antibody or antigen binding fragment thereof comprises: (a) a CDR-H1 comprising the amino acid sequence set forth in SEQ ID NO: 481; (b) a CDR-H2 comprising the amino acid sequence set forth in SEQ ID NO: 519; (c) a CDR-H3 comprising the amino acid sequence set forth in SEQ ID NO: 544; (d) a CDR-L1 comprising the amino acid sequence set forth in SEQ ID NO: 569; (e) a CDR-L2 comprising the amino acid sequence set forth SEQ ID NO: 594; and/or (f) a CDR-L3 comprising the amino acid sequence set forth SEQ ID NO: 619. In some embodiments, the antibody or antigen binding fragment thereof comprises: (a) a CDR-H1 comprising the amino acid sequence set forth in SEQ ID NO: 483; (b) a CDR-H2 comprising the amino acid sequence set forth in SEQ ID NO: 521; (c) a CDR-H3 comprising the amino acid sequence set forth in SEQ ID NO: 546; (d) a CDR-L1 comprising the amino acid sequence set forth in SEQ ID NO: 571; (e) a CDR-L2 comprising the amino acid sequence set forth SEQ ID NO: 596; and/or (f) a CDR-L3 comprising the amino acid sequence set forth SEQ ID NO: 621. In some embodiments, the antibody or antigen binding fragment thereof comprises: (a) a CDR-H1 comprising the amino acid sequence set forth in SEQ ID NO: 484; (b) a CDR-H2 comprising the amino acid sequence set forth in SEQ ID NO: 522; (c) a CDR-H3 comprising the amino acid sequence set forth in SEQ ID NO: 547; (d) a CDR-L1 comprising the amino acid sequence set forth in SEQ ID NO: 572; (e) a CDR-L2 comprising the amino acid sequence set forth SEQ ID NO: 597; and/or (f) a CDR-L3 comprising the amino acid sequence set forth SEQ ID NO: 622. In some embodiments, the antibody or antigen binding fragment thereof comprises: (a) a CDR-H1 comprising the amino acid sequence set forth in SEQ ID NO: 630; (b) a CDR-H2 comprising the amino acid sequence set forth in SEQ ID NO: 644; (c) a CDR-H3 comprising the amino acid sequence set forth in SEQ ID NO: 658; (d) a CDR-L1 comprising the amino acid sequence set forth in SEQ ID NO: 672; (e) a CDR-L2 comprising the amino acid sequence set forth SEQ ID NO: 686; and/or (f) a CDR-L3 comprising the amino acid sequence set forth SEQ ID NO: 700.

In some embodiments, the antibody or antigen binding fragment thereof comprises: (a) a CDR-H1 comprising the amino acid sequence set forth in SEQ ID NO: 638; (b) a CDR-H2 comprising the amino acid sequence set forth in SEQ ID NO: 652; (c) a CDR-H3 comprising the amino acid sequence set forth in SEQ ID NO: 666; (d) a CDR-L1 comprising the amino acid sequence set forth in SEQ ID NO: 680; (e) a CDR-L2 comprising the amino acid sequence set forth SEQ ID NO: 694; and/or (f) a CDR-L3 comprising the amino acid sequence set forth SEQ ID NO: 708. In some embodiments, the antibody or antigen binding fragment thereof comprises: (a) a CDR-H1 comprising the amino acid sequence set forth in SEQ ID NO: 467; (b) a CDR-H2 comprising the amino acid sequence set forth in SEQ ID NO: 492; (c) a CDR-H3 comprising the amino acid sequence set forth in SEQ ID NO: 530; (d) a CDR-L1 comprising the amino acid sequence set forth in SEQ ID NO: 555; (e) a CDR-L2 comprising the amino acid sequence set forth SEQ ID NO: 580; and/or (f) a CDR-L3 comprising the amino acid sequence set forth SEQ ID NO: 605. In some embodiments, the antibody or antigen binding fragment thereof comprises: (a) a CDR-H1 comprising the amino acid sequence set forth in SEQ ID NO: 465; (b) a CDR-H2 comprising the amino acid sequence set forth in SEQ ID NO: 490; (c) a CDR-H3 comprising the amino acid sequence set forth in SEQ ID NO: 528; (d) a CDR-L1 comprising the amino acid sequence set forth in SEQ ID NO: 553; (e) a CDR-L2 comprising the amino acid sequence set forth SEQ ID NO: 578; and/or (f) a CDR-L3 comprising the amino acid sequence set forth SEQ ID NO: 603. In some embodiments, the antibody or antigen binding fragment thereof comprises: (a) a CDR-H1 comprising the amino acid sequence set forth in SEQ ID NO: 466; (b) a CDR-H2 comprising the amino acid sequence set forth in SEQ ID NO: 491; (c) a CDR-H3 comprising the amino acid sequence set forth in SEQ ID NO: 529; (d) a CDR-L1 comprising the amino acid sequence set forth in SEQ ID NO: 554; (e) a CDR-L2 comprising the amino acid sequence set forth SEQ ID NO: 579; and/or (f) a CDR-L3 comprising the amino acid sequence set forth SEQ ID NO: 604. In some embodiments, the antibody or antigen binding fragment thereof comprises: (a) a CDR-H1 comprising the amino acid sequence set forth in SEQ ID NO: 468; (b) a CDR-H2 comprising the amino acid sequence set forth in SEQ ID NO: 493; (c) a CDR-H3 comprising the amino acid sequence set forth in SEQ ID NO: 531; (d) a CDR-L1 comprising the amino acid sequence set forth in SEQ ID NO: 556; (e) a CDR-L2 comprising the amino acid sequence set forth SEQ ID NO: 581; and/or (f) a CDR-L3 comprising the amino acid sequence set forth SEQ ID NO: 606. In some embodiments, the antibody or antigen binding fragment thereof comprises: (a) a CDR-H1 comprising the amino acid sequence set forth in SEQ ID NO: 469; (b) a CDR-H2 comprising the amino acid sequence set forth in SEQ ID NO: 494; (c) a CDR-H3 comprising the amino acid sequence set forth in SEQ ID NO: 532; (d) a CDR-L1 comprising the amino acid sequence set forth in SEQ ID NO: 557; (e) a CDR-L2 comprising the amino acid sequence set forth SEQ ID NO: 582; and/or (f) a CDR-L3 comprising the amino acid sequence set forth SEQ ID NO: 607. In some embodiments, the antibody or antigen binding fragment thereof comprises: (a) a CDR-H1 comprising the amino acid sequence set forth in SEQ ID NO: 631; (b) a CDR-H2 comprising the amino acid sequence set forth in SEQ ID NO: 645; (c) a CDR-H3 comprising the amino acid sequence set forth in SEQ ID NO: 659; (d) a CDR-L1 comprising the amino acid sequence set forth in SEQ ID NO: 673; (e) a CDR-L2 comprising the amino acid sequence set forth SEQ ID NO: 687; and/or (f) a CDR-L3 comprising the amino acid sequence set forth SEQ ID NO: 701.

In some embodiments, the antibody or antigen binding fragment thereof comprises: (a) a CDR-H1 comprising the amino acid sequence set forth in SEQ ID NO: 639; (b) a CDR-H2 comprising the amino acid sequence set forth in SEQ ID NO: 653; (c) a CDR-H3 comprising the amino acid sequence set forth in SEQ ID NO: 667; (d) a CDR-L1 comprising the amino acid sequence set forth in SEQ ID NO: 681; (e) a CDR-L2 comprising the amino acid sequence set forth SEQ ID NO: 695; and/or (f) a CDR-L3 comprising the amino acid sequence set forth SEQ ID NO: 709. In some embodiments, the antibody or antigen binding fragment thereof comprises: (a) a CDR-H1 comprising the amino acid sequence set forth in SEQ ID NO: 472; (b) a CDR-H2 comprising the amino acid sequence set forth in SEQ ID NO: 497; (c) a CDR-H3 comprising the amino acid sequence set forth in SEQ ID NO: 535; (d) a CDR-L1 comprising the amino acid sequence set forth in SEQ ID NO: 560; (e) a CDR-L2 comprising the amino acid sequence set forth SEQ ID NO: 585; and/or (f) a CDR-L3 comprising the amino acid sequence set forth SEQ ID NO: 610. In some embodiments, the antibody or antigen binding fragment thereof comprises: (a) a CDR-H1 comprising the amino acid sequence set forth in SEQ ID NO: 470; (b) a CDR-H2 comprising the amino acid sequence set forth in SEQ ID NO: 495; (c) a CDR-H3 comprising the amino acid sequence set forth in SEQ ID NO: 533; (d) a CDR-L1 comprising the amino acid sequence set forth in SEQ ID NO: 558; (e) a CDR-L2 comprising the amino acid sequence set forth SEQ ID NO: 583; and/or (f) a CDR-L3 comprising the amino acid sequence set forth SEQ ID NO: 608. In some embodiments, the antibody or antigen binding fragment thereof comprises: (a) a CDR-H1 comprising the amino acid sequence set forth in SEQ ID NO: 471; (b) a CDR-H2 comprising the amino acid sequence set forth in SEQ ID NO: 496; (c) a CDR-H3 comprising the amino acid sequence set forth in SEQ ID NO: 534; (d) a CDR-L1 comprising the amino acid sequence set forth in SEQ ID NO: 559; (e) a CDR-L2 comprising the amino acid sequence set forth SEQ ID NO: 584; and/or (f) a CDR-L3 comprising the amino acid sequence set forth SEQ ID NO: 609. In some embodiments, the antibody or antigen binding fragment thereof comprises: (a) a CDR-H1 comprising the amino acid sequence set forth in SEQ ID NO: 473; (b) a CDR-H2 comprising the amino acid sequence set forth in SEQ ID NO: 498; (c) a CDR-H3 comprising the amino acid sequence set forth in SEQ ID NO: 536; (d) a CDR-L1 comprising the amino acid sequence set forth in SEQ ID NO: 561; (e) a CDR-L2 comprising the amino acid sequence set forth SEQ ID NO: 586; and/or (f) a CDR-L3 comprising the amino acid sequence set forth SEQ ID NO: 611. In some embodiments, the antibody or antigen binding fragment thereof comprises: (a) a CDR-H1 comprising the amino acid sequence set forth in SEQ ID NO: 474; (b) a CDR-H2 comprising the amino acid sequence set forth in SEQ ID NO: 499; (c) a CDR-H3 comprising the amino acid sequence set forth in SEQ ID NO: 537; (d) a CDR-L1 comprising the amino acid sequence set forth in SEQ ID NO: 562; (e) a CDR-L2 comprising the amino acid sequence set forth SEQ ID NO: 587; and/or (f) a CDR-L3 comprising the amino acid sequence set forth SEQ ID NO: 612. In some embodiments, the antibody or antigen binding fragment thereof comprises: (a) a CDR-H1 comprising the amino acid sequence set forth in SEQ ID NO: 632; (b) a CDR-H2 comprising the amino acid sequence set forth in SEQ ID NO: 646; (c) a CDR-H3 comprising the amino acid sequence set forth in SEQ ID NO: 660; (d) a CDR-L1 comprising the amino acid sequence set forth in SEQ ID NO: 674; (e) a CDR-L2 comprising the amino acid sequence set forth SEQ ID NO: 688; and/or (f) a CDR-L3 comprising the amino acid sequence set forth SEQ ID NO: 702.

In some embodiments, the antibody or antigen binding fragment thereof comprises: (a) a CDR-H1 comprising the amino acid sequence set forth in SEQ ID NO: 640; (b) a CDR-H2 comprising the amino acid sequence set forth in SEQ ID NO: 654; (c) a CDR-H3 comprising the amino acid sequence set forth in SEQ ID NO: 668; (d) a CDR-L1 comprising the amino acid sequence set forth in SEQ ID NO: 682; (e) a CDR-L2 comprising the amino acid sequence set forth SEQ ID NO: 696; and/or (f) a CDR-L3 comprising the amino acid sequence set forth SEQ ID NO: 710. In some embodiments, the antibody or antigen binding fragment thereof comprises: (a) a CDR-H1 comprising the amino acid sequence set forth in SEQ ID NO: 477; (b) a CDR-H2 comprising the amino acid sequence set forth in SEQ ID NO: 515; (c) a CDR-H3 comprising the amino acid sequence set forth in SEQ ID NO: 540; (d) a CDR-L1 comprising the amino acid sequence set forth in SEQ ID NO: 565; (e) a CDR-L2 comprising the amino acid sequence set forth SEQ ID NO: 590; and/or (f) a CDR-L3 comprising the amino acid sequence set forth SEQ ID NO: 615. In some embodiments, the antibody or antigen binding fragment thereof comprises: (a) a CDR-H1 comprising the amino acid sequence set forth in SEQ ID NO: 475; (b) a CDR-H2 comprising the amino acid sequence set forth in SEQ ID NO: 513; (c) a CDR-H3 comprising the amino acid sequence set forth in SEQ ID NO: 538; (d) a CDR-L1 comprising the amino acid sequence set forth in SEQ ID NO: 563; (e) a CDR-L2 comprising the amino acid sequence set forth SEQ ID NO: 588; and/or (f) a CDR-L3 comprising the amino acid sequence set forth SEQ ID NO: 613. In some embodiments, the antibody or antigen binding fragment thereof comprises: (a) a CDR-H1 comprising the amino acid sequence set forth in SEQ ID NO: 476; (b) a CDR-H2 comprising the amino acid sequence set forth in SEQ ID NO: 514; (c) a CDR-H3 comprising the amino acid sequence set forth in SEQ ID NO: 539; (d) a CDR-L1 comprising the amino acid sequence set forth in SEQ ID NO: 564; (e) a CDR-L2 comprising the amino acid sequence set forth SEQ ID NO: 589; and/or (f) a CDR-L3 comprising the amino acid sequence set forth SEQ ID NO: 614. In some embodiments, the antibody or antigen binding fragment thereof comprises: (a) a CDR-H1 comprising the amino acid sequence set forth in SEQ ID NO: 478; (b) a CDR-H2 comprising the amino acid sequence set forth in SEQ ID NO: 516; (c) a CDR-H3 comprising the amino acid sequence set forth in SEQ ID NO: 541; (d) a CDR-L1 comprising the amino acid sequence set forth in SEQ ID NO: 566; (e) a CDR-L2 comprising the amino acid sequence set forth SEQ ID NO: 591; and/or (f) a CDR-L3 comprising the amino acid sequence set forth SEQ ID NO: 616. In some embodiments, the antibody or antigen binding fragment thereof comprises: (a) a CDR-H1 comprising the amino acid sequence set forth in SEQ ID NO: 479; (b) a CDR-H2 comprising the amino acid sequence set forth in SEQ ID NO: 517; (c) a CDR-H3 comprising the amino acid sequence set forth in SEQ ID NO: 542; (d) a CDR-L1 comprising the amino acid sequence set forth in SEQ ID NO: 567; (e) a CDR-L2 comprising the amino acid sequence set forth SEQ ID NO: 592; and/or (f) a CDR-L3 comprising the amino acid sequence set forth SEQ ID NO: 617. In some embodiments, the antibody or antigen binding fragment thereof comprises: (a) a CDR-H1 comprising the amino acid sequence set forth in SEQ ID NO: 633; (b) a CDR-H2 comprising the amino acid sequence set forth in SEQ ID NO: 647; (c) a CDR-H3 comprising the amino acid sequence set forth in SEQ ID NO: 661; (d) a CDR-L1 comprising the amino acid sequence set forth in SEQ ID NO: 675; (e) a CDR-L2 comprising the amino acid sequence set forth SEQ ID NO: 689; and/or (f) a CDR-L3 comprising the amino acid sequence set forth SEQ ID NO: 703.

In some embodiments, the antibody or antigen binding fragment thereof comprises: (a) a CDR-H1 comprising the amino acid sequence set forth in SEQ ID NO: 641; (b) a CDR-H2 comprising the amino acid sequence set forth in SEQ ID NO: 655; (c) a CDR-H3 comprising the amino acid sequence set forth in SEQ ID NO: 669; (d) a CDR-L1 comprising the amino acid sequence set forth in SEQ ID NO: 683; (e) a CDR-L2 comprising the amino acid sequence set forth SEQ ID NO: 697; and/or (f) a CDR-L3 comprising the amino acid sequence set forth SEQ ID NO: 711. In some embodiments, the antibody or antigen binding fragment thereof comprises: (a) a CDR-H1 comprising the amino acid sequence set forth in SEQ ID NO: 487; (b) a CDR-H2 comprising the amino acid sequence set forth in SEQ ID NO: 525; (c) a CDR-H3 comprising the amino acid sequence set forth in SEQ ID NO: 550; (d) a CDR-L1 comprising the amino acid sequence set forth in SEQ ID NO: 575; (e) a CDR-L2 comprising the amino acid sequence set forth SEQ ID NO: 600; and/or (f) a CDR-L3 comprising the amino acid sequence set forth SEQ ID NO: 625. In some embodiments, the antibody or antigen binding fragment thereof comprises: (a) a CDR-H1 comprising the amino acid sequence set forth in SEQ ID NO: 485; (b) a CDR-H2 comprising the amino acid sequence set forth in SEQ ID NO: 523; (c) a CDR-H3 comprising the amino acid sequence set forth in SEQ ID NO: 548; (d) a CDR-L1 comprising the amino acid sequence set forth in SEQ ID NO: 573; (e) a CDR-L2 comprising the amino acid sequence set forth SEQ ID NO: 598; and/or (f) a CDR-L3 comprising the amino acid sequence set forth SEQ ID NO: 623. In some embodiments, the antibody or antigen binding fragment thereof comprises: (a) a CDR-H1 comprising the amino acid sequence set forth in SEQ ID NO: 486; (b) a CDR-H2 comprising the amino acid sequence set forth in SEQ ID NO: 524; (c) a CDR-H3 comprising the amino acid sequence set forth in SEQ ID NO: 549; (d) a CDR-L1 comprising the amino acid sequence set forth in SEQ ID NO: 574; (e) a CDR-L2 comprising the amino acid sequence set forth SEQ ID NO: 599; and/or (f) a CDR-L3 comprising the amino acid sequence set forth SEQ ID NO: 624. In some embodiments, the antibody or antigen binding fragment thereof comprises: (a) a CDR-H1 comprising the amino acid sequence set forth in SEQ ID NO: 488; (b) a CDR-H2 comprising the amino acid sequence set forth in SEQ ID NO: 526; (c) a CDR-H3 comprising the amino acid sequence set forth in SEQ ID NO: 551; (d) a CDR-L1 comprising the amino acid sequence set forth in SEQ ID NO: 576; (e) a CDR-L2 comprising the amino acid sequence set forth SEQ ID NO: 601; and/or (f) a CDR-L3 comprising the amino acid sequence set forth SEQ ID NO: 626. In some embodiments, the antibody or antigen binding fragment thereof comprises: (a) a CDR-H1 comprising the amino acid sequence set forth in SEQ ID NO: 489; (b) a CDR-H2 comprising the amino acid sequence set forth in SEQ ID NO: 527; (c) a CDR-H3 comprising the amino acid sequence set forth in SEQ ID NO: 552; (d) a CDR-L1 comprising the amino acid sequence set forth in SEQ ID NO: 577; (e) a CDR-L2 comprising the amino acid sequence set forth SEQ ID NO: 602; and/or (f) a CDR-L3 comprising the amino acid sequence set forth SEQ ID NO: 627. In some embodiments, the antibody or antigen binding fragment thereof comprises: (a) a CDR-H1 comprising the amino acid sequence set forth in SEQ ID NO: 634; (b) a CDR-H2 comprising the amino acid sequence set forth in SEQ ID NO: 648; (c) a CDR-H3 comprising the amino acid sequence set forth in SEQ ID NO: 662; (d) a CDR-L1 comprising the amino acid sequence set forth in SEQ ID NO: 676; (e) a CDR-L2 comprising the amino acid sequence set forth SEQ ID NO: 690; and/or (f) a CDR-L3 comprising the amino acid sequence set forth SEQ ID NO: 704.

In some embodiments, the antibody or antigen binding fragment thereof comprises: (a) an immunoglobulin variable region heavy chain (VH) comprising an amino acid sequence having at least about 90, 95, 97, 98, 99, or 100% sequence identity to any one of SEQ ID NOs: 1, 2, 5, 6, 9, 10, 13, 14, 17, 19, 21, 23, 25, 27, 29, and 31; and/or (b) an immunoglobulin variable region light chain (VL) comprising an amino acid sequence having at least about 90, 95, 97, 98, 99, or 100% sequence identity to any one of SEQ ID NOs: 3, 4, 7, 8, 11, 12, 15, 16, 18, 20, 22, 24, 26, 28, and 30. In some embodiments, the antibody or antigen binding fragment thereof comprises: (a) a VH comprising an amino acid sequence having at least about 90, 95, 97, 98, 99, or 100% sequence identity to SEQ ID NO: 1; and/or (b) a VL comprising an amino acid sequence having at least about 90, 95, 97, 98, 99, or 100% sequence identity to SEQ ID NO: 3. In some embodiments, the antibody or antigen binding fragment thereof comprises: (a) a VH comprising an amino acid sequence having at least about 90, 95, 97, 98, 99, or 100% sequence identity to SEQ ID NO: 2; and/or (b) a VL comprising an amino acid sequence having at least about 90, 95, 97, 98, 99, or 100% sequence identity to SEQ ID NO: 4. In some embodiments, the antibody or antigen binding fragment thereof comprises: (a) a VH comprising an amino acid sequence having at least about 90, 95, 97, 98, 99, or 100% sequence identity to SEQ ID NO: 5; and/or (b) a VL comprising an amino acid sequence having at least about 90, 95, 97, 98, 99, or 100% sequence identity to SEQ ID NO: 7. In some embodiments, the antibody or antigen binding fragment thereof comprises: (a) a VH comprising an amino acid sequence having at least about 90, 95, 97, 98, 99, or 100% sequence identity to SEQ ID NO: 6; and/or (b) a VL comprising an amino acid sequence having at least about 90, 95, 97, 98, 99, or 100% sequence identity to SEQ ID NO: 8. In some embodiments, the antibody or antigen binding fragment thereof comprises: (a) a VH comprising an amino acid sequence having at least about 90, 95, 97, 98, 99, or 100% sequence identity to SEQ ID NO: 9; and/or (b) a VL comprising an amino acid sequence having at least about 90, 95, 97, 98, 99, or 100% sequence identity to SEQ ID NO: 11. In some embodiments, the antibody or antigen binding fragment thereof comprises: (a) a VH comprising an amino acid sequence having at least about 90, 95, 97, 98, 99, or 100% sequence identity to SEQ ID NO: 10; and/or (b) a VL comprising an amino acid sequence having at least about 90, 95, 97, 98, 99, or 100% sequence identity to SEQ ID NO: 12. In some embodiments, the antibody or antigen binding fragment thereof comprises: (a) a VH comprising an amino acid sequence having at least about 90, 95, 97, 98, 99, or 100% sequence identity to SEQ ID NO: 13; and/or (b) a VL comprising an amino acid sequence having at least about 90, 95, 97, 98, 99, or 100% sequence identity to SEQ ID NO: 15. In some embodiments, the antibody or antigen binding fragment thereof comprises: (a) a VH comprising an amino acid sequence having at least about 90, 95, 97, 98, 99, or 100% sequence identity to SEQ ID NO: 14; and/or (b) a VL comprising an amino acid sequence having at least about 90, 95, 97, 98, 99, or 100% sequence identity to SEQ ID NO: 16. In some embodiments, the antibody or antigen binding fragment thereof comprises: (a) a VH comprising an amino acid sequence having at least about 90, 95, 97, 98, 99, or 100% sequence identity to SEQ ID NO: 23; and/or (b) a VL comprising an amino acid sequence having at least about 90, 95, 97, 98, 99, or 100% sequence identity to SEQ ID NO: 24. In some embodiments, the antibody or antigen binding fragment thereof comprises: (a) a VH comprising an amino acid sequence having at least about 90, 95, 97, 98, 99, or 100% sequence identity to SEQ ID NO: 25; and/or (b) a VL comprising an amino acid sequence having at least about 90, 95, 97, 98, 99, or 100% sequence identity to SEQ ID NO: 26. In some embodiments, the antibody or antigen binding fragment thereof comprises: (a) a VH comprising an amino acid sequence having at least about 90, 95, 97, 98, 99, or 100% sequence identity to SEQ ID NO: 27; and/or (b) a VL comprising an amino acid sequence having at least about 90, 95, 97, 98, 99, or 100% sequence identity to SEQ ID NO: 28. In some embodiments, the antibody or antigen binding fragment thereof comprises: (a) a VH comprising an amino acid sequence having at least about 90, 95, 97, 98, 99, or 100% sequence identity to SEQ ID NO: 29; and/or (b) a VL comprising an amino acid sequence having at least about 90, 95, 97, 98, 99, or 100% sequence identity to SEQ ID NO: 30. In some embodiments, the antibody or antigen binding fragment thereof comprises: (a) a VH comprising an amino acid sequence having at least about 90, 95, 97, 98, 99, or 100% sequence identity to SEQ ID NO: 31; and/or (b) a VL comprising an amino acid sequence having at least about 90, 95, 97, 98, 99, or 100% sequence identity to SEQ ID NO: 32.

In some embodiments, the antibody or antigen binding fragment thereof comprises: (a) a VH comprising an amino acid sequence set forth in any one of SEQ ID NOs: 1, 2, 5, 6, 9, 10, 13, 14, 17, 19, 21, 23, 25, 27, 29, and 31; and/or (b) a VL comprising an amino acid sequence set forth in any one of SEQ ID NOs: 3, 4, 7, 8, 11, 12, 15, 16, 18, 20, 22, 24, 26, 28, and 30. In some embodiments, the antibody or antigen binding fragment thereof comprises: (a) a VH comprising an amino acid sequence set forth in SEQ ID NO: 1; and/or (b) a VL comprising an amino acid sequence set forth in SEQ ID NO: 3. In some embodiments, the antibody or antigen binding fragment thereof comprises: (a) a VH comprising an amino acid sequence set forth in SEQ ID NO: 2; and/or (b) a VL comprising an amino acid sequence set forth in SEQ ID NO: 4. In some embodiments, the antibody or antigen binding fragment thereof comprises: (a) a VH comprising an amino acid sequence set forth in SEQ ID NO: 5; and/or (b) a VL comprising an amino acid sequence set forth in SEQ ID NO: 7. In some embodiments, the antibody or antigen binding fragment thereof comprises: (a) a VH comprising an amino acid sequence set forth in SEQ ID NO: 6; and/or (b) a VL comprising an amino acid sequence set forth in SEQ ID NO: 8. In some embodiments, the antibody or antigen binding fragment thereof comprises: (a) a VH comprising an amino acid sequence set forth in SEQ ID NO: 9; and/or (b) a VL comprising an amino acid sequence set forth in SEQ ID NO: 11. In some embodiments, the antibody or antigen binding fragment thereof comprises: (a) a VH comprising an amino acid sequence set forth in SEQ ID NO: 10; and/or (b) a VL comprising an amino acid sequence set forth in SEQ ID NO: 12. In some embodiments, the antibody or antigen binding fragment thereof comprises: (a) a VH comprising an amino acid sequence set forth in SEQ ID NO: 13; and/or (b) a VL comprising an amino acid sequence set forth in SEQ ID NO: 15. In some embodiments, the antibody or antigen binding fragment thereof comprises: (a) a VH comprising an amino acid sequence set forth in SEQ ID NO: 14; and/or (b) a VL comprising an amino acid sequence set forth in SEQ ID NO: 16. In some embodiments, the antibody or antigen binding fragment thereof comprises: (a) a VH comprising an amino acid sequence set forth in SEQ ID NO: 23; and/or (b) a VL comprising an amino acid sequence set forth in SEQ ID NO: 24. In some embodiments, the antibody or antigen binding fragment thereof comprises: (a) a VH comprising an amino acid sequence set forth in SEQ ID NO: 25; and/or (b) a VL comprising an amino acid sequence set forth in SEQ ID NO: 26. In some embodiments, the antibody or antigen binding fragment thereof comprises: (a) a VH comprising an amino acid sequence set forth in SEQ ID NO: 27; and/or (b) a VL comprising an amino acid sequence set forth in SEQ ID NO: 28. In some embodiments, the antibody or antigen binding fragment thereof comprises: (a) a VH comprising an amino acid sequence set forth in SEQ ID NO: 29; and/or (b) a VL comprising an amino acid sequence set forth in SEQ ID NO: 30. In some embodiments, the antibody or antigen binding fragment thereof comprises: (a) a VH comprising an amino acid sequence set forth in SEQ ID NO: 31; and/or (b) a VL comprising an amino acid sequence set forth in SEQ ID NO: 32.

In some embodiments, the antibody or antigen binding fragment thereof further comprises an IgG constant region. In some embodiments, the antibody or antigen binding fragment thereof further comprises an IgG constant region having reduced antibody-dependent cell-mediated cytotoxicity (ADCC) function as compared to human IgG and/or reduced complement-dependent cytotoxicity (CDC) as compared to human IgG. In some embodiments, the constant region comprises an amino acid sequence having 80, 85, 90, 95, 97, 98, 99, or 100% sequence identity to the amino acid sequence set forth by any one of SEQ ID NOs: 500-512. In some embodiments, the constant region comprises the amino acid sequence set forth by any one of SEQ ID NOs: 500-512.

In some embodiments, the antibody or antigen binding fragment thereof further comprises a constant region having an amino acid sequence variant corresponding to (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(mmm), per EU numbering.

In some embodiments, the antibody or antigen binding fragment thereof is an IgG antibody. In some embodiments, the IgG antibody is IgG1, IgG2, IgG3, or IgG4.

In some embodiments, the antibody or antigen binding fragment thereof is human, chimeric, or humanized.

In some embodiments, the antigen binding fragment thereof is a Fab, F(ab′)₂, a single-domain antibody, or a single chain variable fragment (scFv).

In some embodiments, the antibody or antigen binding fragment thereof binds one or more amino acids residues of CD30L that interact with CD30.

In some embodiments, the antibody or antigen binding fragment thereof inhibits a binding interaction between CD30L and CD30. In some embodiments, the antibody or antigen binding fragment thereof blocks a binding interaction between CD30L and CD30. In some embodiments, the inhibition or blocking is determined in an ELISA assay, a cell binding assay with CD30L expressing cells, or a surface plasmon resonance (SPR) assay.

In some embodiments, the antibody or antigen binding fragment thereof specifically binds to CD30L.

In some embodiments, the antibody or antigen binding fragment thereof (i) inhibits interleukin-8 secretion in a cell-based assay, (ii) inhibits interleukin-6 secretion in a cell-based assay, or (iii) both (i) and (ii). In some embodiments, the antibody or antigen binding fragment thereof (i) blocks interleukin-8 secretion in a cell-based assay, (ii) blocks interleukin-6 secretion in a cell-based assay, or (iii) both (i) and (ii). In some embodiments, the cell-based assay is a dual cell assay with a cell expressing CD30 and a cell expressing CD30L.

In some embodiments, the antibody or antigen binding fragment thereof binds to (i) human CD30L, (ii) cynomolgus CD30L, or (iii) both human CD30L and cynomolgus CD30L.

In some embodiments, the antibody or antigen binding fragment thereof binds to CD30L with a dissociation equilibrium constant (K_{D}) of no more than 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, or 1000 pM. In some embodiments, the antibody or antigen binding fragment thereof binds to CD30L with an association rate constant (kₒₙ) of at least 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 1.1×10⁶, 1.2×10⁶, 1.3×10⁶, 1.4×10⁶, 1.5×10⁶, or 1.55×10⁶M⁻¹ S⁻¹. In some embodiments, the antibody or antigen binding fragment thereof binds to CD30L with a dissociation rate constant (k_{off}) of no more than 1.4×10⁻⁴, 1.41×10⁻⁴, 1.5×10⁻⁴, 1.6×10⁻⁴, 1.7×10⁻⁴, 1.8×10⁻⁴, 1.9×10⁻⁴, 2.0×10⁻⁴, 2.1×10⁻⁴, 2.2×10⁻⁴, 2.3×10⁻⁴, 2.4×10⁻⁴, 2.5×10⁻⁴, 2.6×10⁻⁴, 2.7×10⁻⁴, 2.8×10⁻⁴, 2.9×10⁻⁴, 3.0×10⁻⁴, 3.1×10⁻⁴, 3.2×10⁻⁴, 3.3×10⁻⁴, 3.4×10⁻⁴, or 3.5×10⁻⁴ S⁻¹.

In some embodiments, the antibody or antigen binding fragment thereof is recombinant antibody or antigen binding fragment thereof.

In some embodiments, the antibody or antigen binding fragment thereof is isolated antibody or antigen binding fragment thereof.

In one aspect, provided herein are recombinant antibodies and/or antigen binding fragments thereof that binds CD30L, wherein the antibody or antigen binding fragment thereof comprises: (a) an immunoglobulin heavy chain CDR1 (CDR-H1) comprising the amino acid sequence set forth in any one of SEQ ID NOs: 100-139 or 220-234; (b) an immunoglobulin heavy chain CDR2 (CDR-H2) comprising the amino acid sequence set forth in any one of SEQ ID NOs: 140-179 or 235-249; (c) an immunoglobulin heavy chain CDR3 (CDR-H3) comprising the amino acid sequence set forth in any one of SEQ ID NOs: 180-219 or 250-264; (d) an immunoglobulin light chain CDR1 (CDR-L1) comprising the amino acid sequence set forth in any one of SEQ ID NOs: 300-339 or 420-434; (e) an immunoglobulin light chain CDR2 (CDR-L2) comprising the amino acid sequence set forth in any one of SEQ ID NOs: 340-379 or 435-449; and/or (f) an immunoglobulin light chain CDR3 (CDR-L3) comprising the amino acid sequence set forth in any one of SEQ ID NOs: 380-419 or 450-464.

In some embodiments, provided is an antibody or antigen binding fragment thereof of any of the preceding embodiments, wherein the antibody or antigen binding fragment thereof comprises: (a) an immunoglobulin heavy chain CDR1 (CDR-H1) comprising the amino acid sequence set forth in any one of SEQ ID NOs: 100-109; (b) an immunoglobulin heavy chain CDR2 (CDR-H2) comprising the amino acid sequence set forth in any one of SEQ ID NOs: 140-149; (c) an immunoglobulin heavy chain CDR3 (CDR-H3) comprising the amino acid sequence set forth in any one of SEQ ID NOs: 180-189; (d) an immunoglobulin light chain CDR1 (CDR-L1) comprising the amino acid sequence set forth in any one of SEQ ID NOs: 300-309; (e) an immunoglobulin light chain CDR2 (CDR-L2) comprising the amino acid sequence set forth in any one of SEQ ID NOs: 340-349; and/or (f) an immunoglobulin light chain CDR3 (CDR-L3) comprising the amino acid sequence set forth in any one of SEQ ID NOs: 380-389.

In some embodiments, provided is an antibody or antigen binding fragment thereof of any of the preceding embodiments, wherein the antibody or antigen binding fragment thereof comprises: (a) an immunoglobulin heavy chain CDR1 (CDR-H1) comprising the amino acid sequence set forth in any one of SEQ ID NOs: 100-104; (b) an immunoglobulin heavy chain CDR2 (CDR-H2) comprising the amino acid sequence set forth in any one of SEQ ID NOs: 140-144; (c) an immunoglobulin heavy chain CDR3 (CDR-H3) comprising the amino acid sequence set forth in any one of SEQ ID NOs: 180-184; (d) an immunoglobulin light chain CDR1 (CDR-L1) comprising the amino acid sequence set forth in any one of SEQ ID NOs: 300-304; (e) an immunoglobulin light chain CDR2 (CDR-L2) comprising the amino acid sequence set forth in any one of SEQ ID NOs: 340-344; and/or (f) an immunoglobulin light chain CDR3 (CDR-L3) comprising the amino acid sequence set forth in any one of SEQ ID NOs: 380-384.

In some embodiments, provided is an antibody or antigen binding fragment thereof of any of the preceding embodiments, wherein the antibody or antigen binding fragment thereof comprises: (a) an immunoglobulin heavy chain CDR1 (CDR-H1) comprising the amino acid sequence set forth in any one of SEQ ID NOs: 105-109; (b) an immunoglobulin heavy chain CDR2 (CDR-H2) comprising the amino acid sequence set forth in any one of SEQ ID NOs: 145-149; (c) an immunoglobulin heavy chain CDR3 (CDR-H3) comprising the amino acid sequence set forth in any one of SEQ ID NOs: 185-189; (d) an immunoglobulin light chain CDR1 (CDR-L1) comprising the amino acid sequence set forth in any one of SEQ ID NOs: 305-309; (e) an immunoglobulin light chain CDR2 (CDR-L2) comprising the amino acid sequence set forth in any one of SEQ ID NOs: 345-349; and/or (f) an immunoglobulin light chain CDR3 (CDR-L3) comprising the amino acid sequence set forth in any one of SEQ ID NOs: 385-389.

In some embodiments, provided is an antibody or antigen binding fragment thereof of any of the preceding embodiments, comprising an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to any one of SEQ ID NOs: 1 and 2; and/or (b) the immunoglobulin variable region light chain comprises an amino acid sequence having at least about 90, 95, 97, 98, 99, or 100% sequence identity to any one of SEQ ID NOs: 3 and 4.

In some embodiments, provided is an antibody or antigen binding fragment thereof of any of the preceding embodiments, wherein the antibody or antigen binding fragment thereof comprises: (a) an immunoglobulin heavy chain CDR1 (CDR-H1) comprising the amino acid sequence set forth in any one of SEQ ID NOs: 110-119; (b) an immunoglobulin heavy chain CDR2 (CDR-H2) comprising the amino acid sequence set forth in any one of SEQ ID NOs: 150-159; (c) an immunoglobulin heavy chain CDR3 (CDR-H3) comprising the amino acid sequence set forth in any one of SEQ ID NOs: 190-199; (d) an immunoglobulin light chain CDR1 (CDR-L1) comprising the amino acid sequence set forth in any one of SEQ ID NOs: 310-319; (e) an immunoglobulin light chain CDR2 (CDR-L2) comprising the amino acid sequence set forth in any one of SEQ ID NOs: 350-359; and/or (e) an immunoglobulin light chain CDR3 (CDR-L3) comprising the amino acid sequence set forth in any one of SEQ ID NOs: 390-399.

In some embodiments, provided is an antibody or antigen binding fragment thereof of any of the preceding embodiments, wherein the antibody or antigen binding fragment thereof comprises: (a) an immunoglobulin heavy chain CDR1 (CDR-H1) comprising the amino acid sequence set forth in any one of SEQ ID NOs: 110-114; (b) an immunoglobulin heavy chain CDR2 (CDR-H2) comprising the amino acid sequence set forth in any one of SEQ ID NOs: 150-154; (c) an immunoglobulin heavy chain CDR3 (CDR-H3) comprising the amino acid sequence set forth in any one of SEQ ID NOs: 190-194; (d) an immunoglobulin light chain CDR1 (CDR-L1) comprising the amino acid sequence set forth in any one of SEQ ID NOs: 310-314; (e) an immunoglobulin light chain CDR2 (CDR-L2) comprising the amino acid sequence set forth in any one of SEQ ID NOs: 350-354; and/or (e) an immunoglobulin light chain CDR3 (CDR-L3) comprising the amino acid sequence set forth in any one of SEQ ID NOs: 390-394.

In some embodiments, provided is an antibody or antigen binding fragment thereof of any of the preceding embodiments, wherein the antibody or antigen binding fragment thereof comprises: (a) an immunoglobulin heavy chain CDR1 (CDR-H1) comprising the amino acid sequence set forth in any one of SEQ ID NOs: 115-119; (b) an immunoglobulin heavy chain CDR2 (CDR-H2) comprising the amino acid sequence set forth in any one of SEQ ID NOs: 155-159; (c) an immunoglobulin heavy chain CDR3 (CDR-H3) comprising the amino acid sequence set forth in any one of SEQ ID NOs: 195-199; (d) an immunoglobulin light chain CDR1 (CDR-L1) comprising the amino acid sequence set forth in any one of SEQ ID NOs: 315-319; (e) an immunoglobulin light chain CDR2 (CDR-L2) comprising the amino acid sequence set forth in any one of SEQ ID NOs: 355-359; and/or (f) an immunoglobulin light chain CDR3 (CDR-L3) comprising the amino acid sequence set forth in any one of SEQ ID NOs: 395-399.

In some embodiments, provided is an antibody or antigen binding fragment thereof of any of the preceding embodiments, comprising an immunoglobulin variable region heavy chain and an immunoglobulin variable region light chain, wherein (a) the immunoglobulin variable region heavy chain comprises an amino acid sequence having at least about 90, 95, 97, 98, 99, or 100% sequence identity to any one of SEQ ID NOs: 5 and 6; and/or (b) the immunoglobulin variable region light chain comprises an amino acid sequence having at least about 90, 95, 97, 98, 99, or 100% sequence identity to any one of SEQ ID NOs: 7 and 8.

In some embodiments, provided is an antibody or antigen binding fragment thereof of any of the preceding embodiments, wherein the antibody or antigen binding fragment thereof comprises: (a) an immunoglobulin heavy chain CDR1 (CDR-H1) comprising the amino acid sequence set forth in any one of SEQ ID NOs: 120-129; (b) an immunoglobulin heavy chain CDR2 (CDR-H2) comprising the amino acid sequence set forth in any one of SEQ ID NOs: 160-169; (c) an immunoglobulin heavy chain CDR3 (CDR-H3) comprising the amino acid sequence set forth in any one of SEQ ID NOs: 200-209; (d) an immunoglobulin light chain CDR1 (CDR-L1) comprising the amino acid sequence set forth in any one of SEQ ID NOs: 320-329; (e) an immunoglobulin light chain CDR2 (CDR-L2) comprising the amino acid sequence set forth in any one of SEQ ID NOs: 360-369; and/or (f) an immunoglobulin light chain CDR3 (CDR-L3) comprising the amino acid sequence set forth in any one of SEQ ID NOs: 400-409.

In some embodiments, provided is an antibody or antigen binding fragment thereof of any of the preceding embodiments, wherein the antibody or antigen binding fragment thereof comprises: (a) an immunoglobulin heavy chain CDR1 (CDR-H1) comprising the amino acid sequence set forth in any one of SEQ ID NOs: 120-124; (b) an immunoglobulin heavy chain CDR2 (CDR-H2) comprising the amino acid sequence set forth in any one of SEQ ID NOs: 160-164; (c) an immunoglobulin heavy chain CDR3 (CDR-H3) comprising the amino acid sequence set forth in any one of SEQ ID NOs: 200-204; (d) an immunoglobulin light chain CDR1 (CDR-L1) comprising the amino acid sequence set forth in any one of SEQ ID NOs: 320-324; (e) an immunoglobulin light chain CDR2 (CDR-L2) comprising the amino acid sequence set forth in any one of SEQ ID NOs: 360-364; and/or (f) an immunoglobulin light chain CDR3 (CDR-L3) comprising the amino acid sequence set forth in any one of SEQ ID NOs: 400-404.

In some embodiments, provided is an antibody or antigen binding fragment thereof of any of the preceding embodiments, wherein the antibody or antigen binding fragment thereof comprises: (a) an immunoglobulin heavy chain CDR1 (CDR-H1) comprising the amino acid sequence set forth in any one of SEQ ID NOs: 125-129; (b) an immunoglobulin heavy chain CDR2 (CDR-H2) comprising the amino acid sequence set forth in any one of SEQ ID NOs: 165-169; (c) an immunoglobulin heavy chain CDR3 (CDR-H3) comprising the amino acid sequence set forth in any one of SEQ ID NOs: 205-209; (d) an immunoglobulin light chain CDR1 (CDR-L1) comprising the amino acid sequence set forth in any one of SEQ ID NOs: 325-329; (e) an immunoglobulin light chain CDR2 (CDR-L2) comprising the amino acid sequence set forth in any one of SEQ ID NOs: 365-369; and/or (f) an immunoglobulin light chain CDR3 (CDR-L3) comprising the amino acid sequence set forth in any one of SEQ ID NOs: 405-409.

In some embodiments, provided is an antibody or antigen binding fragment thereof of any of the preceding embodiments, comprising an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to any one of SEQ ID NOs: 9 and 10; and/or (b) the immunoglobulin variable region light chain comprises an amino acid sequence having at least about 90, 95, 97, 98, 99, or 100% sequence identity to any one of SEQ ID NOs: 11 and 12.

In some embodiments, provided is an antibody or antigen binding fragment thereof of any of the preceding embodiments, wherein the antibody or antigen binding fragment thereof comprises: (a) an immunoglobulin heavy chain CDR1 (CDR-H1) comprising the amino acid sequence set forth in any one of SEQ ID NOs: 130-139; (b) an immunoglobulin heavy chain CDR2 (CDR-H2) comprising the amino acid sequence set forth in any one of SEQ ID NOs: 170-179; (c) an immunoglobulin heavy chain CDR3 (CDR-H3) comprising the amino acid sequence set forth in any one of SEQ ID NOs: 210-219; (d) an immunoglobulin light chain CDR1 (CDR-L1) comprising the amino acid sequence set forth in any one of SEQ ID NOs: 330-339; (e) an immunoglobulin light chain CDR2 (CDR-L2) comprising the amino acid sequence set forth in any one of SEQ ID NOs: 370-379; and/or (f) an immunoglobulin light chain CDR3 (CDR-L3) comprising the amino acid sequence set forth in any one of SEQ ID NOs: 410-419.

In some embodiments, provided is an antibody or antigen binding fragment thereof of any of the preceding embodiments, wherein the antibody or antigen binding fragment thereof comprises: (a) an immunoglobulin heavy chain CDR1 (CDR-H1) comprising the amino acid sequence set forth in any one of SEQ ID NOs: 130-134; (b) an immunoglobulin heavy chain CDR2 (CDR-H2) comprising the amino acid sequence set forth in any one of SEQ ID NOs: 170-174; (c) an immunoglobulin heavy chain CDR3 (CDR-H3) comprising the amino acid sequence set forth in any one of SEQ ID NOs: 210-214; (d) an immunoglobulin light chain CDR1 (CDR-L1) comprising the amino acid sequence set forth in any one of SEQ ID NOs: 330-334; (e) an immunoglobulin light chain CDR2 (CDR-L2) comprising the amino acid sequence set forth in any one of SEQ ID NOs: 370-374; and/or (f) an immunoglobulin light chain CDR3 (CDR-L3) comprising the amino acid sequence set forth in any one of SEQ ID NOs: 410-414.

In some embodiments, provided is an antibody or antigen binding fragment thereof of any of the preceding embodiments, wherein the antibody or antigen binding fragment thereof comprises: (a) an immunoglobulin heavy chain CDR1 (CDR-H1) comprising the amino acid sequence set forth in any one of SEQ ID NOs: 135-139; (b) an immunoglobulin heavy chain CDR2 (CDR-H2) comprising the amino acid sequence set forth in any one of SEQ ID NOs: 175-179; (c) an immunoglobulin heavy chain CDR3 (CDR-H3) comprising the amino acid sequence set forth in any one of SEQ ID NOs: 215-219; (d) an immunoglobulin light chain CDR1 (CDR-L1) comprising the amino acid sequence set forth in any one of SEQ ID NOs: 335-339; (e) an immunoglobulin light chain CDR2 (CDR-L2) comprising the amino acid sequence set forth in any one of SEQ ID NOs: 375-379; and/or (f) an immunoglobulin light chain CDR3 (CDR-L3) comprising the amino acid sequence set forth in any one of SEQ ID NOs: 415-419.

In some embodiments, provided is an antibody or antigen binding fragment thereof of any of the preceding embodiments, comprising an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to any one of SEQ ID NOs: 13 and 14; and/or (b) the immunoglobulin variable region light chain comprises an amino acid sequence having at least about 90, 95, 97, 98, 99, or 100% sequence identity to any one of SEQ ID NOs: 15 and 16.

In some embodiments, provided is an antibody or antigen binding fragment thereof of any of the preceding embodiments, wherein the antibody or antigen binding fragment thereof comprises: (a) an immunoglobulin heavy chain CDR1 (CDR-H1) comprising the amino acid sequence set forth in any one of SEQ ID NOs: 220-224; (b) an immunoglobulin heavy chain CDR2 (CDR-H2) comprising the amino acid sequence set forth in any one of SEQ ID NOs: 235-239; (c) an immunoglobulin heavy chain CDR3 (CDR-H3) comprising the amino acid sequence set forth in any one of SEQ ID NOs: 250-254; (d) an immunoglobulin light chain CDR1 (CDR-L1) comprising the amino acid sequence set forth in any one of SEQ ID NOs: 420-424; (e) an immunoglobulin light chain CDR2 (CDR-L2) comprising the amino acid sequence set forth in any one of SEQ ID NOs: 435-439; and/or (f) an immunoglobulin light chain CDR3 (CDR-L3) comprising the amino acid sequence set forth in any one of SEQ ID NOs: 450-454.

In some embodiments, provided is an antibody or antigen binding fragment thereof of any of the preceding embodiments, comprising an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to any one of SEQ ID NO: 17; and/or (b) the immunoglobulin variable region light chain comprises an amino acid sequence having at least about 90, 95, 97, 98, 99, or 100% sequence identity to any one of SEQ ID NO: 15 and 18.

In some embodiments, provided is an antibody or antigen binding fragment thereof of any of the preceding embodiments, wherein the antibody or antigen binding fragment thereof comprises: (a) an immunoglobulin heavy chain CDR1 (CDR-H1) comprising the amino acid sequence set forth in any one of SEQ ID NOs: 225-229; (b) an immunoglobulin heavy chain CDR2 (CDR-H2) comprising the amino acid sequence set forth in any one of SEQ ID NOs: 240-244; (c) an immunoglobulin heavy chain CDR3 (CDR-H3) comprising the amino acid sequence set forth in any one of SEQ ID NOs: 255-259; (d) an immunoglobulin light chain CDR1 (CDR-L1) comprising the amino acid sequence set forth in any one of SEQ ID NOs: 425-429; (e) an immunoglobulin light chain CDR2 (CDR-L2) comprising the amino acid sequence set forth in any one of SEQ ID NOs: 440-444; and/or (f) an immunoglobulin light chain CDR3 (CDR-L3) comprising the amino acid sequence set forth in any one of SEQ ID NOs: 455-459.

In some embodiments, provided is an antibody or antigen binding fragment thereof of any of the preceding embodiments, comprising an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to any one of SEQ ID NO: 19; and/or (b) the immunoglobulin variable region light chain comprises an amino acid sequence having at least about 90, 95, 97, 98, 99, or 100% sequence identity to any one of SEQ ID NO: 15 and 20.

In some embodiments, provided is an antibody or antigen binding fragment thereof of any of the preceding embodiments, wherein the antibody or antigen binding fragment thereof comprises: (a) an immunoglobulin heavy chain CDR1 (CDR-H1) comprising the amino acid sequence set forth in any one of SEQ ID NOs: 230-234; (b) an immunoglobulin heavy chain CDR2 (CDR-H2) comprising the amino acid sequence set forth in any one of SEQ ID NOs: 245-249; (c) an immunoglobulin heavy chain CDR3 (CDR-H3) comprising the amino acid sequence set forth in any one of SEQ ID NOs: 260-264; (d) an immunoglobulin light chain CDR1 (CDR-L1) comprising the amino acid sequence set forth in any one of SEQ ID NOs: 430-434; (e) an immunoglobulin light chain CDR2 (CDR-L2) comprising the amino acid sequence set forth in any one of SEQ ID NOs: 445-449; and/or (f) an immunoglobulin light chain CDR3 (CDR-L3) comprising the amino acid sequence set forth in any one of SEQ ID NOs: 460-464.

In some embodiments, provided is an antibody or antigen binding fragment thereof of any of the preceding embodiments, comprising an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to any one of SEQ ID NO: 21; and/or (b) the immunoglobulin variable region light chain comprises an amino acid sequence having at least about 90, 95, 97, 98, 99, or 100% sequence identity to any one of SEQ ID NO: 15 and 22.

In some embodiments, provided is an antibody or antigen binding fragment thereof of any of the preceding embodiments, comprising a constant region (e.g. a fragment crystallizable (Fc) region) having reduced antibody-dependent cell-mediated cytotoxicity (ADCC) function as compared to human IgG1 and/or reduced complement-dependent cytotoxicity (CDC) as compared to human IgG1. In some embodiments, provided is an antibody or antigen binding fragment thereof of any of the preceding embodiments, wherein the Constant region comprises an amino acid sequence having 80, 85, 90, 95, 97, 98, 99, or 100% sequence identity to the amino acid sequence set forth by any one of SEQ ID NOs: 500-512. In some embodiments, provided is an antibody or antigen binding fragment thereof of any of the preceding embodiments, wherein the Constant region comprises the amino acid sequence set forth by any one of SEQ ID NOs: 500-512.

In some embodiments, provided is an antibody or antigen binding fragment thereof of any of the preceding embodiments, comprising a constant region having an amino acid sequence variant corresponding to (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(mmm), per EU numbering.

In some embodiments, provided is an antibody or antigen binding fragment thereof of any of the preceding embodiments, wherein the recombinant antibody or antigen binding fragment thereof is an IgG antibody. In some embodiments, provided is an antibody or antigen binding fragment thereof of any of the preceding embodiments, wherein the IgG antibody is IgG1, IgG2, IgG3, or IgG4. In some embodiments, provided is an antibody or antigen binding fragment thereof of any of the preceding embodiments, wherein the recombinant antibody or antigen binding fragment thereof is human, chimeric, or humanized.

In some embodiments, provided is an antibody or antigen binding fragment thereof of any of the preceding embodiments, wherein the recombinant antibody or antigen binding fragment thereof is a Fab, F(ab)′₂, a single-domain antibody, or a single chain variable fragment (scFv). In some embodiments, provided is an antibody or antigen binding fragment thereof of any of the preceding embodiments, wherein the recombinant antibody or antigen binding fragment thereof is a bispecific or multispecific antibody.

In some embodiments, provided is an antibody or antigen binding fragment thereof of any of the preceding embodiments, wherein the recombinant antibody or antigen binding fragment thereof inhibits a binding interaction between CD30L and CD30.

In an aspect, further provided are nucleic acids encoding the recombinant antibody or antigen binding fragment thereof of any of the preceding embodiments. Also provided are cells comprising the recombinant antibody or antigen binding fragment thereof of any of the preceding embodiments. In some embodiments, the cell is a eukaryotic cell. In some embodiments, the cell is a prokaryotic cell.

In an aspect, provided are recombinant antibodies or antigen binding fragments thereof of any the preceding embodiments for use in a method of inhibiting binding of CD30L to CD30. Further provided are recombinant antibodies or antigen binding fragments thereof of any the preceding embodiments for use in a method of inhibiting activation of CD30 signaling in a cell. Also provided are recombinant antibodies or antigen binding fragments thereof of any the preceding embodiments for use in a method of inhibiting activation, expression, and/or secretion of a pro-inflammatory cytokine protein.

In an aspect, provided are recombinant antibodies or antigen binding fragments thereof of any the preceding embodiments for use in treating an autoimmune disease in an individual in need thereof. In some embodiments, the autoimmune disease is irritable bowel disease. In some embodiments, the irritable bowel disease comprises ulcerative colitis (UC) or Crohn's Disease (CD).

In an aspect, provided are methods of treating or ameliorating an autoimmune disease in an individual in need thereof, the method comprising administering to the individual the recombinant antibody or antigen binding fragment thereof of any of the preceding embodiments, thereby treating or ameliorating the autoimmune disease. Also provided are methods for inhibiting and/or reducing binding of CD30L to CD30 in an individual with an inflammatory or autoimmune disorder, the method comprising administering to an individual afflicted with the inflammatory or autoimmune disorder the recombinant antibody or antigen binding fragment thereof of the preceding embodiments, thereby inhibiting and/or reducing the binding of CD30L to CD30. Further provided are methods of reducing and/or inhibiting inflammation in an individual, the method comprising administering to the individual the recombinant antibody or antigen binding fragment thereof of any of the preceding embodiments, thereby reducing and/or inhibiting inflammation.

In some embodiments, the provided are methods of any of the preceding embodiments, wherein the individual has an autoimmune disease. In some embodiments, the provided are methods of any of the preceding embodiments, wherein the autoimmune disease is irritable bowel disease. In some embodiments, the provided are methods of any of the preceding embodiments, wherein the irritable bowel disease comprises ulcerative colitis (UC) or Crohn's Disease (CD).

In some embodiments, the provided are methods of any of the preceding embodiments, wherein reducing and/or inhibiting inflammation comprises reducing an amount of pro-inflammatory cytokine expression or secretion in the individual or a tissue of the individual. In some embodiments, the provided are methods of any of the preceding embodiments, wherein the pro-inflammatory cytokine comprises interleukin 8 and/or interleukin 6. In some embodiments, the provided are methods of any of the preceding embodiments, wherein the individual has an autoimmune disease. In some embodiments, the provided are methods of any of the preceding embodiments, wherein the autoimmune disease is irritable bowel disease. In some embodiments, the provided are methods of any of the preceding embodiments, wherein the irritable bowel disease comprises ulcerative colitis (UC) or Crohn's Disease (CD).

Further provided herein are pharmaceutical compositions comprising antibodies that bind CD30 ligand (also referred to as CD30L, CD153, TNFSF8), which pharmaceutical compositions are useful in the treatment of autoimmune disorders such as IBD. Generally, in an aspect, the pharmaceutical compositions provided herein comprise anti-CD30L that effectively inhibit an interaction between CD30L and CD30 in order to effectively reduce, inhibit, or prevent immune activation (for example, an inflammatory response). Furthermore, the anti-CD30L in the pharmaceutical compositions provided herein also has properties useful for therapeutic application, including, for example, reduced and/or low immunogenicity. The pharmaceutical compositions comprising anti-CD30L described herein thus can inhibit expression/secretion of certain inflammatory cytokines (e.g., interleukin-6 and interleukin-8) that play a key role in inflammatory and autoimmune diseases such as inflammatory bowel disease, Crohn's disease, and ulcerative colitis.

Accordingly, in one aspect, provided herein is a pharmaceutical composition comprising an antibody or antigen binding fragment thereof that binds CD30L (anti-CD30L antibody or antigen binding fragment), wherein the antibody or antigen binding fragment thereof comprises: (a) a CDR-H1 comprising the amino acid sequence set forth in any one of SEQ ID NOs: 100-139, 220-234, 465-489, 628-641, and 712-723; (b) a CDR-H2 comprising the amino acid sequence set forth in any one of SEQ ID NOs: 140-179, 235-249, 490-499, 513-527, 642-655, and 724-735; (c) a CDR-H3 comprising the amino acid sequence set forth in any one of SEQ Id NOs: 180-219, 250-264, 528-552, 656-669, and 736-743; (d) a CDR-L1 comprising the amino acid sequence set forth in any one of SEQ ID NOs: 300-339, 420-434, 553-577, 670-683, and 744-751; (d) CDR-L2 comprising the amino acid sequence set forth in any one of SEQ ID NOs: 340-379, 435-449, 578-602, 684-697, and 752-759; and/or (e) a CDR-L3 comprising the amino acid sequence set forth in any one of SEQ ID NOs: 380-419, 450-464, 603-627, 698-711, and 760-765.

In some embodiments of the pharmaceutical composition of the anti-CD30L antibody or antigen binding fragment provided herein, the antibody or antigen binding fragment thereof comprises: (a) a CDR-H1 comprising the amino acid sequence set forth in any one of SEQ ID NOs: 100-109, 628, 635, and 712-723; (b) a CDR-H2 comprising the amino acid sequence set forth in any one of SEQ ID NOs: 140-149, 642, 649, and 724-735; (c) a CDR-H3 comprising the amino acid sequence set forth in any one of SEQ ID NOs: 180-189, 656, 663, and 736-743; (d) a CDR-L1 comprising the amino acid sequence set forth in any one of SEQ ID NOs: 300-309, 670, 677, and 744-751; (e) a CDR-L2 comprising the amino acid sequence set forth in any one of SEQ ID NOs: 340-349, 684, 691, and 752-759; and/or (f) a CDR-L3 comprising the amino acid sequence set forth in any one of SEQ ID Nos: 380-389, 698, 705, and 760-765.

In some embodiments of the pharmaceutical composition of the anti-CD30L antibody or antigen binding fragment provided herein, the antibody or antigen binding fragment thereof comprises: (a) a CDR-H1 comprising the amino acid sequence set forth in any one of SEQ ID NOs: 100-104; (b) a CDR-H2 comprising the amino acid sequence set forth in any one of SEQ ID NOs: 140-144; (c) a CDR-H3 comprising the amino acid sequence set forth in any one of SEQ ID NOs: 180-184; (d) a CDR-L1 comprising the amino acid sequence set forth in any one of SEQ ID NOs: 300-304; (e) a CDR-L2 comprising the amino acid sequence set forth in any one of SEQ ID NOs: 340-344; and/or (f) a CDR-L3 comprising the amino acid sequence set forth in any one of SEQ ID NOs: 380-384.

In some embodiments of the pharmaceutical composition of the anti-CD30L antibody or antigen binding fragment provided herein, the antibody or antigen binding fragment thereof comprises: (a) a CDR-H1 comprising the amino acid sequence set forth in any one of SEQ ID NOs: 105-109, 628, 635, and 712-723; (b) a CDR-H2 comprising the amino acid sequence set forth in any one of SEQ ID NOs: 145-149, 642, 649, and 724-735; (c) a CDR-H3 comprising the amino acid sequence set forth in any one of SEQ ID NOs: 185-189, 656, 663, and 736-743; (d) a CDR-L1 comprising the amino acid sequence set forth in any one of SEQ ID NOs: 305-309, 670, 677, and 744-751; (e) a CDR-L2 comprising the amino acid sequence set forth in any one of SEQ ID NOs: 345-349, 684, 691, and 752-759; and/or (f) a CDR-L3 comprising the amino acid sequence set forth in any one of SEQ ID NOs: 385-389, 698, 705, and 760-765.

In some embodiments of the pharmaceutical composition of the anti-CD30L antibody or antigen binding fragment provided herein, the antibody or antigen binding fragment thereof comprises an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to any one of SEQ ID NOs: 1 and 2; and/or (b) the immunoglobulin variable region light chain comprises an amino acid sequence having at least about 90, 95, 97, 98, 99, or 100% sequence identity to any one of SEQ ID NOs: 3 and 4.

In one aspect, provided herein is a pharmaceutical composition comprising an antibody or antigen binding fragment thereof that binds CD30L (anti-CD30L antibody or antigen binding fragment), wherein the antibody or antigen binding fragment thereof binds to an epitope comprising one or more amino acids in CD30L selected from the group consisting of N165, K166, I168, K169, and D234, wherein the amino acids are numbered according to the amino acid sequence of CD30L as set forth in SEQ ID NO:33.

In some embodiments of the pharmaceutical composition of the anti-CD30L antibody or antigen binding fragment provided herein, the antibody or antigen binding fragment thereof binds to an epitope comprising: (i) any one amino acid in CD30L selected from the group consisting of N165, K166, I168, K169, and D234; (ii) any two amino acids in CD30L selected from the group consisting of N165, K166, I168, K169, and D234; (iii) any three amino acids in CD30L selected from the group consisting of N165, K166, I168, K169, and D234; (iv) any four amino acids in CD30L selected from the group consisting of N165, K166, I168, K169, and D234; or (v) any five amino acids in CD30L selected from the group consisting of N165, K166, I168, K169, and D234; wherein the amino acids are numbered according to the amino acid sequence of CD30L as set forth in SEQ ID NO:33.

In another aspect, provided herein is a pharmaceutical composition comprising an antibody or antigen binding fragment thereof that binds CD30L (anti-CD30L antibody or antigen binding fragment), wherein the antibody or antigen binding fragment thereof binds to an epitope comprising one or more amino acids in CD30L selected from the group consisting of H167, S217, and D118, wherein the amino acids are numbered according to the amino acid sequence of CD30L as set forth in SEQ ID NO:33.

In some embodiments of the pharmaceutical composition of the anti-CD30L antibody or antigen binding fragment provided herein, the antibody or antigen binding fragment thereof binds to an epitope comprising: (i) any one amino acid in CD30L selected from the group consisting of H167, S217, and D118; (ii) any two amino acids in CD30L selected from the group consisting of H167, S217, and D118; or (iii) any three amino acids in CD30L selected from the group consisting of H167, S217, and D118; wherein the amino acids are numbered according to the amino acid sequence of CD30L as set forth in SEQ ID NO:33.

In yet another aspect, provided herein is a pharmaceutical composition comprising an antibody or antigen binding fragment thereof that binds CD30L (anti-CD30L antibody or antigen binding fragment), wherein the antibody or antigen binding fragment thereof binds to an epitope comprising one or more amino acids in CD30L selected from the group consisting of D118, N165, K166, H167, I168, K169, S217, and D234, wherein the amino acids are numbered according to the amino acid sequence of CD30L as set forth in SEQ ID NO:33.

In some embodiments of the pharmaceutical composition of the anti-CD30L antibody or antigen binding fragment provided herein, the antibody or antigen binding fragment thereof binds to an epitope comprising: (i) any one amino acid in CD30L selected from the group consisting of D118, N165, K166, H167, I168, K169, S217, and D234; (ii) any two amino acids in CD30L selected from the group consisting of D118, N165, K166, H167, I168, K169, S217, and D234; (iii) any three amino acids in CD30L selected from the group consisting of D118, N165, K166, H167, I168, K169, S217, and D234; (iv) any four amino acids in CD30L selected from the group consisting of D118, N165, K166, H167, I168, K169, S217, and D234; (v) any five amino acids in CD30L selected from the group consisting of D118, N165, K166, H167, I168, K169, S217, and D234; (vi) any six amino acids in CD30L selected from the group consisting of D118, N165, K166, H167, I168, K169, S217, and D234; (vii) any seven amino acids in CD30L selected from the group consisting of D118, N165, K166, H167, I168, K169, S217, and D234; or (viii) any five amino acids in CD30L selected from the group consisting of D118, N165, K166, H167, I168, K169, S217, and D234; wherein the amino acids are numbered according to the amino acid sequence of CD30L as set forth in SEQ ID NO:33.

In a further aspect, provided herein is a pharmaceutical composition comprising an anti-CD30L antibody or antigen binding fragment thereof, wherein the antibody or antigen binding fragment thereof binds to an epitope comprising K169 in CD30L, wherein the amino acids are numbered according to the amino acid sequence of CD30L as set forth in SEQ ID NO: 33.

In some embodiments of the pharmaceutical composition of the anti-CD30L antibody or antigen binding fragment provided herein, the antibody or antigen binding fragment thereof binds to an epitope comprising D234 in CD30L, wherein the amino acids are numbered according to the amino acid sequence of CD30L as set forth in SEQ ID NO:33.

In one aspect, provided herein is a pharmaceutical composition comprising an anti-CD30L antibody or antigen binding fragment thereof, wherein the antibody or antigen binding fragment thereof binds to an epitope comprising D234 in CD30L, wherein the amino acids are numbered according to the amino acid sequence of CD30L as set forth in SEQ ID NO: 33

In some embodiments of the pharmaceutical composition of the anti-CD30L antibody or antigen binding fragment provided herein, the epitope of the anti-CD30L antibody or antigen binding fragment further comprises N165 in CD30L, wherein the amino acids are numbered according to the amino acid sequence of CD30L as set forth in SEQ ID NO:33.

In some embodiments of the pharmaceutical composition of the anti-CD30L antibody or antigen binding fragment provided herein, the epitope of the anti-CD30L antibody or antigen binding fragment further comprises I168 in CD30L, wherein the amino acids are numbered according to the amino acid sequence of CD30L as set forth in SEQ ID NO:33.

In some embodiments of the pharmaceutical composition of the anti-CD30L antibody or antigen binding fragment provided herein, the epitope of the anti-CD30L antibody or antigen binding fragment further comprises K166 in CD30L, wherein the amino acids are numbered according to the amino acid sequence of CD30L as set forth in SEQ ID NO:33.

In some embodiments of the pharmaceutical composition of the anti-CD30L antibody or antigen binding fragment provided herein, the epitope of the anti-CD30L antibody or antigen binding fragment further comprises H167 in CD30L, wherein the amino acids are numbered according to the amino acid sequence of CD30L as set forth in SEQ ID NO: 33.

In some embodiments of the pharmaceutical composition of the anti-CD30L antibody or antigen binding fragment provided herein, the epitope of the anti-CD30L antibody or antigen binding fragment further comprises S217 in CD30L, wherein the amino acids are numbered according to the amino acid sequence of CD30L as set forth in SEQ ID NO: 33.

In some embodiments of the pharmaceutical composition of the anti-CD30L antibody or antigen binding fragment provided herein, the epitope of the anti-CD30L antibody or antigen binding fragment further comprises D118 in CD30L, wherein the amino acids are numbered according to the amino acid sequence of CD30L as set forth in SEQ ID NO: 33.

In some embodiments of the pharmaceutical composition of the anti-CD30L antibody or antigen binding fragment provided herein, the antibody or antigen binding fragment thereof comprises: (a) a CDR-H1 comprising the amino acid sequence set forth in any one of SEQ ID NOs: 110-119, 629, 636, and 712-723; (b) a CDR-H2 comprising the amino acid sequence set forth in any one of SEQ ID NOs: 150-159, 643, 650, and 724-735; (c) a CDR-H3 comprising the amino acid sequence set forth in any one of SEQ ID NOs: 190-199, 657, 664, and 736-743; (d) a CDR-L1 comprising the amino acid sequence set forth in any one of SEQ ID NOs: 310-319, 671, 678, and 744-751; (e) a CDR-L2 comprising the amino acid sequence set forth in any one of SEQ ID NOs: 350-359, 685, 692, and 752-759; and/or (f) a CDR-L3 comprising the amino acid sequence set forth in any one of SEQ ID NOs: 390-399, 699, 706, and 760-765.

In some embodiments of the pharmaceutical composition of the anti-CD30L antibody or antigen binding fragment provided herein, the antibody or antigen binding fragment thereof comprises: (a) a CDR-H1 comprising the amino acid sequence set forth in any one of SEQ ID NOs: 110-114; (b) a CDR-H2 comprising the amino acid sequence set forth in any one of SEQ ID NOs: 150-154; (c) a CDR-H3 comprising the amino acid sequence set forth in any one of SEQ ID NOs: 190-194; (d) a CDR-L1 comprising the amino acid sequence set forth in any one of SEQ ID NOs: 310-314; (e) a CDR-L2 comprising the amino acid sequence set forth in any one of SEQ ID NOs: 350-354; and/or (f) a CDR-L3 comprising the amino acid sequence set forth in any one of SEQ ID NOs: 390-394.

In some embodiments of the pharmaceutical composition of the anti-CD30L antibody or antigen binding fragment provided herein, the antibody or antigen binding fragment thereof comprises: (a) a CDR-H1 comprising the amino acid sequence set forth in any one of SEQ ID NOs: 115-119, 629, 636, and 712-723; (b) a CDR-H2 comprising the amino acid sequence set forth in any one of SEQ ID NOs: 155-159, 643, 650, and 724-735; (c) a CDR-H3 comprising the amino acid sequence set forth in any one of SEQ ID NOs: 195-199, 657, 664, and 736-743; (d) a CDR-L1 comprising the amino acid sequence set forth in any one of SEQ ID NOs: 315-319, 671, 678, and 744-751; (e) a CDR-L2 comprising the amino acid sequence set forth in any one of SEQ ID NOs: 355-359, 685, 692, and 752-759; and/or (f) a CDR-L3 comprising the amino acid sequence set forth in any one of SEQ ID NOs: 395-399, 699, 706, and 760-765.

In some embodiments of the pharmaceutical composition of the anti-CD30L antibody or antigen binding fragment provided herein, the antibody or antigen binding fragment thereof comprises an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to any one of SEQ ID NOs: 5 and 6; and/or (b) the immunoglobulin variable region light chain comprises an amino acid sequence having at least about 90, 95, 97, 98, 99, or 100% sequence identity to any one of SEQ ID NOs: 7 and 8.

In some embodiments of the pharmaceutical composition of the anti-CD30L antibody or antigen binding fragment provided herein, the antibody or antigen binding fragment thereof comprises: (a) a CDR-H1 comprising the amino acid sequence set forth in any one of SEQ ID NOs: 120-129, and 712-723; (b) a CDR-H2 comprising the amino acid sequence set forth in any one of SEQ ID NOs: 160-169, and 724-735; (c) a CDR-H3 comprising the amino acid sequence set forth in any one of SEQ ID NOs: 200-209, and 736-743; (d) a CDR-L1 comprising the amino acid sequence set forth in any one of SEQ ID NOs: 320-329, and 744-751; (e) a CDR-L2 comprising the amino acid sequence set forth in any one of SEQ ID NOs: 360-369, and 752-759; and/or (f) a CDR-L3 comprising the amino acid sequence set forth in any one of SEQ ID NOs: 400-409, and 760-765.

In some embodiments of the pharmaceutical composition of the anti-CD30L antibody or antigen binding fragment provided herein, the antibody or antigen binding fragment thereof comprises: (a) a CDR-H1 comprising the amino acid sequence set forth in any one of SEQ ID NOs: 120-124; (b) a CDR-H2 comprising the amino acid sequence set forth in any one of SEQ ID NOs: 160-164; (c) a CDR-H3 comprising the amino acid sequence set forth in any one of SEQ ID NOs: 200-204; (d) a CDR-L1 comprising the amino acid sequence set forth in any one of SEQ ID NOs: 320-324; (e) a CDR-L2 comprising the amino acid sequence set forth in any one of SEQ ID NOs: 360-364; and/or (f) a CDR-L3 comprising the amino acid sequence set forth in any one of SEQ ID NOs: 400-404.

In some embodiments of the pharmaceutical composition of the anti-CD30L antibody or antigen binding fragment provided herein, the antibody or antigen binding fragment thereof comprises: (a) a CDR-H1 comprising the amino acid sequence set forth in any one of SEQ ID NOs: 125-129, and 712-723; (b) a CDR-H2 comprising the amino acid sequence set forth in any one of SEQ ID NOs: 165-169, and 724-735; (c) a CDR-H3 comprising the amino acid sequence set forth in any one of SEQ ID NOs: 205-209, and 736-743; (d) a CDR-L1 comprising the amino acid sequence set forth in any one of SEQ ID NOs: 325-329, and 744-751; (e) a CDR-L2 comprising the amino acid sequence set forth in any one of SEQ ID NOs: 365-369, and 752-759; and/or (f) a CDR-L3 comprising the amino acid sequence set forth in any one of SEQ ID NOs: 405-409, and 760-765.

In some embodiments of the pharmaceutical composition of the anti-CD30L antibody or antigen binding fragment provided herein, the antibody or antigen binding fragment thereof comprises an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to any one of SEQ ID NOs: 9 and 10; and/or (b) the immunoglobulin variable region light chain comprises an amino acid sequence having at least about 90, 95, 97, 98, 99, or 100% sequence identity to any one of SEQ ID NOs: 11 and 12.

In some embodiments of the pharmaceutical composition of the anti-CD30L antibody or antigen binding fragment provided herein, the antibody or antigen binding fragment thereof comprises: (a) a CDR-H1 comprising the amino acid sequence set forth in any one of SEQ ID NOs: 130-139, and 712-723; (b) a CDR-H2 comprising the amino acid sequence set forth in any one of SEQ ID NOs: 170-179, and 724-735; (c) a CDR-H3 comprising the amino acid sequence set forth in any one of SEQ ID NOs: 210-219, and 736-743; (d) a CDR-L1 comprising the amino acid sequence set forth in any one of SEQ ID NOs: 330-339, and 744-751; (e) a CDR-L2 comprising the amino acid sequence set forth in any one of SEQ ID NOs: 370-379, and 752-759; and/or (f) a CDR-L3 comprising the amino acid sequence set forth in any one of SEQ ID NOs: 410-419, and 760-765.

In some embodiments of the pharmaceutical composition of the anti-CD30L antibody or antigen binding fragment provided herein, the antibody or antigen binding fragment thereof comprises: (a) a CDR-H1 comprising the amino acid sequence set forth in any one of SEQ ID NOs: 130-134; (b) a CDR-H2 comprising the amino acid sequence set forth in any one of SEQ ID NOs: 170-174; (c) a CDR-H3 comprising the amino acid sequence set forth in any one of SEQ ID NOs: 210-214; (d) a CDR-L1 comprising the amino acid sequence set forth in any one of SEQ ID NOs: 330-334; (e) a CDR-L2 comprising the amino acid sequence set forth in any one of SEQ ID NOs: 370-374; and/or (f) a CDR-L3 comprising the amino acid sequence set forth in any one of SEQ ID NOs: 410-414.

In some embodiments of the pharmaceutical composition of the anti-CD30L antibody or antigen binding fragment provided herein, the antibody or antigen binding fragment thereof comprises: (a) a CDR-H1 comprising the amino acid sequence set forth in any one of SEQ ID NOs: 135-139, and 712-723; (b) a CDR-H2 comprising the amino acid sequence set forth in any one of SEQ ID NOs: 175-179, and 724-735; (c) a CDR-H3 comprising the amino acid sequence set forth in any one of SEQ ID NOs: 215-219, and 736-743; (d) a CDR-L1 comprising the amino acid sequence set forth in any one of SEQ ID NOs: 335-339, and 744-751; (e) a CDR-L2 comprising the amino acid sequence set forth in any one of SEQ ID NOs: 375-379, and 752-759; and/or (f) a CDR-L3 comprising the amino acid sequence set forth in any one of SEQ ID NOs: 415-419, and 760-765.

In some embodiments of the pharmaceutical composition of the anti-CD30L antibody or antigen binding fragment provided herein, the antibody or antigen binding fragment thereof comprises an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to any one of SEQ ID NOs: 13 and 14; and/or (b) the immunoglobulin variable region light chain comprises an amino acid sequence having at least about 90, 95, 97, 98, 99, or 100% sequence identity to any one of SEQ ID NOs: 15 and 16.

In some embodiments of the pharmaceutical composition of the anti-CD30L antibody or antigen binding fragment provided herein, the antibody or antigen binding fragment thereof comprises: (a) a CDR-H1 comprising the amino acid sequence set forth in any one of SEQ ID NOs: 220-224, and 712-723; (b) a CDR-H2 comprising the amino acid sequence set forth in any one of SEQ ID NOs: 235-239, and 724-735; (c) a CDR-H3 comprising the amino acid sequence set forth in any one of SEQ ID NOs: 250-254, and 736-743; (d) a CDR-L1 comprising the amino acid sequence set forth in any one of SEQ ID NOs: 420-424, and 744-751; (e) a CDR-L2 comprising the amino acid sequence set forth in any one of SEQ ID NOs: 435-439, and 752-759; and/or (f) a CDR-L3 comprising the amino acid sequence set forth in any one of SEQ ID NOs: 450-454, and 760-765.

In some embodiments of the pharmaceutical composition of the anti-CD30L antibody or antigen binding fragment provided herein, the antibody or antigen binding fragment thereof comprises an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to SEQ ID NO: 17; and/or (b) the immunoglobulin variable region light chain comprises an amino acid sequence having at least about 90, 95, 97, 98, 99, or 100% sequence identity to SEQ ID NO: 18.

In some embodiments of the pharmaceutical composition of the anti-CD30L antibody or antigen binding fragment provided herein, the antibody or antigen binding fragment thereof comprises: (a) a CDR-H1 comprising the amino acid sequence set forth in any one of SEQ ID NOs: 225-229, and 712-723; (b) a CDR-H2 comprising the amino acid sequence set forth in any one of SEQ ID NOs: 240-244, and 724-735; (c) a CDR-H3 comprising the amino acid sequence set forth in any one of SEQ ID NOs: 255-259, and 736-743; (d) a CDR-L1 comprising the amino acid sequence set forth in any one of SEQ ID NOs: 425-429, and 744-751; (e) a CDR-L2 comprising the amino acid sequence set forth in any one of SEQ ID NOs: 440-444, and 752-759; and/or (f) a CDR-L3 comprising the amino acid sequence set forth in any one of SEQ ID NOs: 455-459, and 760-765.

In some embodiments of the pharmaceutical composition of the anti-CD30L antibody or antigen binding fragment provided herein, the antibody or antigen binding fragment thereof comprises an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to SEQ ID NO: 19; and/or (b) the immunoglobulin variable region light chain comprises an amino acid sequence having at least about 90, 95, 97, 98, 99, or 100% sequence identity to SEQ ID NO: 20.

In some embodiments of the pharmaceutical composition of the anti-CD30L antibody or antigen binding fragment provided herein, the antibody or antigen binding fragment thereof comprises: (a) a CDR-H1 comprising the amino acid sequence set forth in any one of SEQ ID NOs: 230-234, and 712-723; (b) a CDR-H2 comprising the amino acid sequence set forth in any one of SEQ ID NOs: 245-249, and 724-735; (c) a CDR-H3 comprising the amino acid sequence set forth in any one of SEQ ID NOs: 260-264, and 736-743; (d) a CDR-L1 comprising the amino acid sequence set forth in any one of SEQ ID NOs: 430-434, and 744-751; (e) a CDR-L2 comprising the amino acid sequence set forth in any one of SEQ ID NOs: 445-449, and 752-759; and/or (f) a CDR-L3 comprising the amino acid sequence set forth in any one of SEQ ID NOs: 460-464, and 760-765.

In some embodiments of the pharmaceutical composition of the anti-CD30L antibody or antigen binding fragment provided herein, the antibody or antigen binding fragment thereof comprises an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to SEQ ID NO: 21; and/or (b) the immunoglobulin variable region light chain comprises an amino acid sequence having at least about 90, 95, 97, 98, 99, or 100% sequence identity to SEQ ID NO: 22.

In some embodiments of the pharmaceutical composition of the anti-CD30L antibody or antigen binding fragment provided herein, the antibody or antigen binding fragment thereof comprises:
(i)(a) a CDR-H1 comprising the amino acid sequence set forth in any one of SEQ ID NOs: 465-469, 631, 638, and 712-723; (b) a CDR-H2 comprising the amino acid sequence set forth in any one of SEQ ID NOs: 490-494, 645, 652, and 724-735; (c) a CDR-H3 comprising the amino acid sequence set forth in any one of SEQ ID NOs: 528-532, 659, 666, and 736-743; (d) a CDR-L1 comprising the amino acid sequence set forth in any one of SEQ ID NOs: 553-557, 673, 680, and 744-751; (e) a CDR-L2 comprising the amino acid sequence set forth in any one of SEQ ID NOs: 578-582, 687, 694, and 752-759; and/or (f) a CDR-L3 comprising the amino acid sequence set forth in any one of SEQ ID NOs: 603-607, 701, 708, and 760-765;
(ii)(a) a CDR-H1 comprising the amino acid sequence set forth in any one of SEQ ID NOs: 470-474, 632, 639, and 712-723; (b) a CDR-H2 comprising the amino acid sequence set forth in any one of SEQ ID NOs: 495-499, 646, 653, and 724-735; (c) a CDR-H3 comprising the amino acid sequence set forth in any one of SEQ ID NOs: 533-537, 660, 667, and 736-743; (d) a CDR-L1 comprising the amino acid sequence set forth in any one of SEQ ID NOs: 558-562, 674, 681, and 744-751; (e) a CDR-L2 comprising the amino acid sequence set forth in any one of SEQ ID NOs: 583-587, 688, 695, and 752-759; and/or (f) a CDR-L3 comprising the amino acid sequence set forth in any one of SEQ ID NOs: 608-612, 702, 709, and 760-765;
(iii)(a) a CDR-H1 comprising the amino acid sequence set forth in any one of SEQ ID NOs: 475-479, 633, 640, and 712-723; (b) a CDR-H2 comprising the amino acid sequence set forth in any one of SEQ ID NOs: 513-517, 647, 654, and 724-735; (c) a CDR-H3 comprising the amino acid sequence set forth in any one of SEQ ID NOs: 538-542, 661, 668, and 736-743; (d) a CDR-L1 comprising the amino acid sequence set forth in any one of SEQ ID NOs: 563-567, 675, 682, and 744-751; (e) a CDR-L2 comprising the amino acid sequence set forth in any one of SEQ ID NOs: 588-592, 689, 696, and 752-759; and/or (f) a CDR-L3 comprising the amino acid sequence set forth in any one of SEQ ID NOs: 613-617, 703, 710, and 760-765;
(iv)(a) a CDR-H1 comprising the amino acid sequence set forth in any one of SEQ ID NOs: 480-484, 630, 637, and 712-723; (b) a CDR-H2 comprising the amino acid sequence set forth in any one of SEQ ID NOs: 518-522, 644, 651, and 724-735; (c) a CDR-H3 comprising the amino acid sequence set forth in any one of SEQ ID NOs: 543-547, 658, 665, and 736-743; (d) a CDR-L1 comprising the amino acid sequence set forth in any one of SEQ ID NOs: 568-572, 672, 679, and 744-751; (e) a CDR-L2 comprising the amino acid sequence set forth in any one of SEQ ID NOs: 593-597, 686, 693, and 752-759; and/or (f) a CDR-L3 comprising the amino acid sequence set forth in any one of SEQ ID NOs: 618-622, 700, 707, and 760-765; or
(v)(a) a CDR-H1 comprising the amino acid sequence set forth in any one of SEQ ID NOs: 485-489, 634, 641, and 712-723; (b) a CDR-H2 comprising the amino acid sequence set forth in any one of SEQ ID NOs: 523-527, 648, 655, and 724-735; (c) a CDR-H3 comprising the amino acid sequence set forth in any one of SEQ ID NOs: 548-552, 662, 669, and 736-743; (d) a CDR-L1 comprising the amino acid sequence set forth in any one of SEQ ID NOs: 573-577, 676, 683, and 744-751; (e) a CDR-L2 comprising the amino acid sequence set forth in any one of SEQ ID NOs: 598-602, 690, 697, and 752-759; and/or (f) a CDR-L3 comprising the amino acid sequence set forth in any one of SEQ ID NOs: 623-627, 704, 711, and 760-765.

In some embodiments of the pharmaceutical composition of the anti-CD30L antibody or antigen binding fragment provided herein, the antibody or antigen binding fragment thereof comprises:
(i)(a) a CDR-H1 comprising the amino acid sequence set forth in any one of SEQ ID NOs: 712-723; (b) a CDR-H2 comprising the amino acid sequence set forth in any one of SEQ ID NOs: 724-735; (c) a CDR-H3 comprising the amino acid sequence set forth in any one of SEQ ID NOs: 736-743; (d) a CDR-L1 comprising the amino acid sequence set forth in any one of SEQ ID NOs: 744-751; (e) a CDR-L2 comprising the amino acid sequence set forth in any one of SEQ ID NOs: 752-759; and/or (f) a CDR-L3 comprising the amino acid sequence set forth in any one of SEQ ID NOs: 760-765;
(ii)(a) a CDR-H1 comprising the amino acid sequence set forth in any one of SEQ ID NOs: 712, 714, 716, 718, 720, and 722; (b) a CDR-H2 comprising the amino acid sequence set forth in any one of SEQ ID NOs: 724, 726, 728, 730, 732, and 734; (c) a CDR-H3 comprising the amino acid sequence set forth in any one of SEQ ID NOs: 736, 738, 740, and 742; (d) a CDR-L1 comprising the amino acid sequence set forth in any one of SEQ ID NOs: 744, 746, 748, and 750; (e) a CDR-L2 comprising the amino acid sequence set forth in any one of SEQ ID NOs: 752, 754, 756, and 758; and/or (f) a CDR-L3 comprising the amino acid sequence set forth in any one of SEQ ID NOs: 760, 762, and 764;
(iii)(a) a CDR-H1 comprising the amino acid sequence set forth in any one of SEQ ID NOs: 713, 715, 717, 719, 721, and 723; (b) a CDR-H2 comprising the amino acid sequence set forth in any one of SEQ ID NOs: 725, 727, 729, 731, 733, and 735; (c) a CDR-H3 comprising the amino acid sequence set forth in any one of SEQ ID NOs: 737, 739, 741, and 743; (d) a CDR-L1 comprising the amino acid sequence set forth in any one of SEQ ID NOs: 745, 747, 749, and 751; (e) a CDR-L2 comprising the amino acid sequence set forth in any one of SEQ ID NOs: 753, 755, 757, and 759; and/or (f) a CDR-L3 comprising the amino acid sequence set forth in any one of SEQ ID NOs: 761, 763, and 765;
(iv)(a) a CDR-H1 comprising the amino acid sequence set forth in SEQ ID NO: 712; (b) a CDR-H2 comprising the amino acid sequence set forth in SEQ ID NO: 730; (c) a CDR-H3 comprising the amino acid sequence set forth in SEQ ID NO: 736; (d) a CDR-L1 comprising the amino acid sequence set forth in SEQ ID NO: 744; (e) a CDR-L2 comprising the amino acid sequence set forth SEQ ID NO: 752; and/or (f) a CDR-L3 comprising the amino acid sequence set forth SEQ ID NO: 760;
(v)(a) a CDR-H1 comprising the amino acid sequence set forth in SEQ ID NO: 713; (b) a CDR-H2 comprising the amino acid sequence set forth in SEQ ID NO: 731; (c) a CDR-H3 comprising the amino acid sequence set forth in SEQ ID NO: 737; (d) a CDR-L1 comprising the amino acid sequence set forth in SEQ ID NO: 745; (e) a CDR-L2 comprising the amino acid sequence set forth SEQ ID NO: 753; and/or (f) a CDR-L3 comprising the amino acid sequence set forth SEQ ID NO: 761;
(vi)(a) a CDR-H1 comprising the amino acid sequence set forth in SEQ ID NO: 712; (b) a CDR-H2 comprising the amino acid sequence set forth in SEQ ID NO: 724; (c) a CDR-H3 comprising the amino acid sequence set forth in SEQ ID NO: 736; (d) a CDR-L1 comprising the amino acid sequence set forth in SEQ ID NO: 744; (e) a CDR-L2 comprising the amino acid sequence set forth SEQ ID NO: 752; and/or (f) a CDR-L3 comprising the amino acid sequence set forth SEQ ID NO: 760;
(vii)(a) a CDR-H1 comprising the amino acid sequence set forth in SEQ ID NO: 713; (b) a CDR-H2 comprising the amino acid sequence set forth in SEQ ID NO: 725; (c) a CDR-H3 comprising the amino acid sequence set forth in SEQ ID NO: 737; (d) a CDR-L1 comprising the amino acid sequence set forth in SEQ ID NO: 745; (e) a CDR-L2 comprising the amino acid sequence set forth SEQ ID NO: 753; and/or (f) a CDR-L3 comprising the amino acid sequence set forth SEQ ID NO: 761;
(viii)(a) a CDR-H1 comprising the amino acid sequence set forth in SEQ ID NO: 714; (b) a CDR-H2 comprising the amino acid sequence set forth in SEQ ID NO: 726; (c) a CDR-H3 comprising the amino acid sequence set forth in SEQ ID NO: 736; (d) a CDR-L1 comprising the amino acid sequence set forth in SEQ ID NO: 744; (e) a CDR-L2 comprising the amino acid sequence set forth SEQ ID NO: 752; and/or (f) a CDR-L3 comprising the amino acid sequence set forth SEQ ID NO: 760;
(ix)(a) a CDR-H1 comprising the amino acid sequence set forth in SEQ ID NO: 715; (b) a CDR-H2 comprising the amino acid sequence set forth in SEQ ID NO: 727; (c) a CDR-H3 comprising the amino acid sequence set forth in SEQ ID NO: 737; (d) a CDR-L1 comprising the amino acid sequence set forth in SEQ ID NO: 745; (e) a CDR-L2 comprising the amino acid sequence set forth SEQ ID NO: 753; and/or (f) a CDR-L3 comprising the amino acid sequence set forth SEQ ID NO: 761;
(x)(a) a CDR-H1 comprising the amino acid sequence set forth in SEQ ID NO: 716; (b) a CDR-H2 comprising the amino acid sequence set forth in SEQ ID NO: 728; (c) a CDR-H3 comprising the amino acid sequence set forth in SEQ ID NO: 736; (d) a CDR-L1 comprising the amino acid sequence set forth in SEQ ID NO: 744; (e) a CDR-L2 comprising the amino acid sequence set forth SEQ ID NO: 752; and/or (f) a CDR-L3 comprising the amino acid sequence set forth SEQ ID NO: 760;
(xi)(a) a CDR-H1 comprising the amino acid sequence set forth in SEQ ID NO: 717; (b) a CDR-H2 comprising the amino acid sequence set forth in SEQ ID NO: 729; (c) a CDR-H3 comprising the amino acid sequence set forth in SEQ ID NO: 737; (d) a CDR-L1 comprising the amino acid sequence set forth in SEQ ID NO: 745; (e) a CDR-L2 comprising the amino acid sequence set forth SEQ ID NO: 753; and/or (f) a CDR-L3 comprising the amino acid sequence set forth SEQ ID NO: 761;
(xii)(a) a CDR-H1 comprising the amino acid sequence set forth in SEQ ID NO: 718; (b) a CDR-H2 comprising the amino acid sequence set forth in SEQ ID NO: 730; (c) a CDR-H3 comprising the amino acid sequence set forth in SEQ ID NO: 738; (d) a CDR-L1 comprising the amino acid sequence set forth in SEQ ID NO: 746; (e) a CDR-L2 comprising the amino acid sequence set forth SEQ ID NO: 754; and/or (f) a CDR-L3 comprising the amino acid sequence set forth SEQ ID NO: 762;
(xiii)(a) a CDR-H1 comprising the amino acid sequence set forth in SEQ ID NO: 719; (b) a CDR-H2 comprising the amino acid sequence set forth in SEQ ID NO: 731; (c) a CDR-H3 comprising the amino acid sequence set forth in SEQ ID NO: 739; (d) a CDR-L1 comprising the amino acid sequence set forth in SEQ ID NO: 747; (e) a CDR-L2 comprising the amino acid sequence set forth SEQ ID NO: 755; and/or (f) a CDR-L3 comprising the amino acid sequence set forth SEQ ID NO: 763;
(xiv)(a) a CDR-H1 comprising the amino acid sequence set forth in SEQ ID NO: 720; (b) a CDR-H2 comprising the amino acid sequence set forth in SEQ ID NO: 732; (c) a CDR-H3 comprising the amino acid sequence set forth in SEQ ID NO: 740; (d) a CDR-L1 comprising the amino acid sequence set forth in SEQ ID NO: 748; (e) a CDR-L2 comprising the amino acid sequence set forth SEQ ID NO: 756; and/or (f) a CDR-L3 comprising the amino acid sequence set forth SEQ ID NO: 760;
(xv)(a) a CDR-H1 comprising the amino acid sequence set forth in SEQ ID NO: 721; (b) a CDR-H2 comprising the amino acid sequence set forth in SEQ ID NO: 733; (c) a CDR-H3 comprising the amino acid sequence set forth in SEQ ID NO: 741; (d) a CDR-L1 comprising the amino acid sequence set forth in SEQ ID NO: 749; (e) a CDR-L2 comprising the amino acid sequence set forth SEQ ID NO: 757; and/or (f) a CDR-L3 comprising the amino acid sequence set forth SEQ ID NO: 761;
(xvi)(a) a CDR-H1 comprising the amino acid sequence set forth in SEQ ID NO: 722; (b) a CDR-H2 comprising the amino acid sequence set forth in SEQ ID NO: 734; (c) a CDR-H3 comprising the amino acid sequence set forth in SEQ ID NO: 742; (d) a CDR-L1 comprising the amino acid sequence set forth in SEQ ID NO: 750; (e) a CDR-L2 comprising the amino acid sequence set forth SEQ ID NO: 758; and/or (f) a CDR-L3 comprising the amino acid sequence set forth SEQ ID NO: 764; or
(xvii)(a) a CDR-H1 comprising the amino acid sequence set forth in SEQ ID NO: 723; (b) a CDR-H2 comprising the amino acid sequence set forth in SEQ ID NO: 735; (c) a CDR-H3 comprising the amino acid sequence set forth in SEQ ID NO: 743; (d) a CDR-L1 comprising the amino acid sequence set forth in SEQ ID NO: 751; (e) a CDR-L2 comprising the amino acid sequence set forth SEQ ID NO: 759; and/or (f) a CDR-L3 comprising the amino acid sequence set forth SEQ ID NO: 765.

In some embodiments of the pharmaceutical composition of the anti-CD30L antibody or antigen binding fragment provided herein, the antibody or antigen binding fragment thereof comprises:
(i)(a) a CDR-H1 comprising the amino acid sequence set forth in SEQ ID NO: 635; (b) a CDR-H2 comprising the amino acid sequence set forth in SEQ ID NO: 649; (c) a CDR-H3 comprising the amino acid sequence set forth in SEQ ID NO: 663; (d) a CDR-L1 comprising the amino acid sequence set forth in SEQ ID NO: 677; (e) a CDR-L2 comprising the amino acid sequence set forth SEQ ID NO: 691; and/or (f) a CDR-L3 comprising the amino acid sequence set forth SEQ ID NO: 705;
(ii)(a) a CDR-H1 comprising the amino acid sequence set forth in SEQ ID NO: 107; (b) a CDR-H2 comprising the amino acid sequence set forth in SEQ ID NO: 147; (c) a CDR-H3 comprising the amino acid sequence set forth in SEQ ID NO: 187; (d) a CDR-L1 comprising the amino acid sequence set forth in SEQ ID NO: 307; (e) a CDR-L2 comprising the amino acid sequence set forth SEQ ID NO: 347; and/or (f) a CDR-L3 comprising the amino acid sequence set forth SEQ ID NO: 387;
(iii)(a) a CDR-H1 comprising the amino acid sequence set forth in SEQ ID NO: 105; (b) a CDR-H2 comprising the amino acid sequence set forth in SEQ ID NO: 145; (c) a CDR-H3 comprising the amino acid sequence set forth in SEQ ID NO: 185; (d) a CDR-L1 comprising the amino acid sequence set forth in SEQ ID NO: 305; (e) a CDR-L2 comprising the amino acid sequence set forth SEQ ID NO: 345; and/or (f) a CDR-L3 comprising the amino acid sequence set forth SEQ ID NO: 385;
(iv)(a) a CDR-H1 comprising the amino acid sequence set forth in SEQ ID NO: 106; (b) a CDR-H2 comprising the amino acid sequence set forth in SEQ ID NO: 146; (c) a CDR-H3 comprising the amino acid sequence set forth in SEQ ID NO: 186; (d) a CDR-L1 comprising the amino acid sequence set forth in SEQ ID NO: 306; (e) a CDR-L2 comprising the amino acid sequence set forth SEQ ID NO: 346; and/or (f) a CDR-L3 comprising the amino acid sequence set forth SEQ ID NO: 386;
(v)(a) a CDR-H1 comprising the amino acid sequence set forth in SEQ ID NO: 108; (b) a CDR-H2 comprising the amino acid sequence set forth in SEQ ID NO: 148; (c) a CDR-H3 comprising the amino acid sequence set forth in SEQ ID NO: 188; (d) a CDR-L1 comprising the amino acid sequence set forth in SEQ ID NO: 308; (e) a CDR-L2 comprising the amino acid sequence set forth SEQ ID NO: 348; and/or (f) a CDR-L3 comprising the amino acid sequence set forth SEQ ID NO: 388;
(vi)(a) a CDR-H1 comprising the amino acid sequence set forth in SEQ ID NO: 109; (b) a CDR-H2 comprising the amino acid sequence set forth in SEQ ID NO: 149; (c) a CDR-H3 comprising the amino acid sequence set forth in SEQ ID NO: 189; (d) a CDR-L1 comprising the amino acid sequence set forth in SEQ ID NO: 309; (e) a CDR-L2 comprising the amino acid sequence set forth SEQ ID NO: 349; and/or (f) a CDR-L3 comprising the amino acid sequence set forth SEQ ID NO: 389; or
(vii)(a) a CDR-H1 comprising the amino acid sequence set forth in SEQ ID NO: 628; (b) a CDR-H2 comprising the amino acid sequence set forth in SEQ ID NO: 642; (c) a CDR-H3 comprising the amino acid sequence set forth in SEQ ID NO: 656; (d) a CDR-L1 comprising the amino acid sequence set forth in SEQ ID NO: 670; (e) a CDR-L2 comprising the amino acid sequence set forth SEQ ID NO: 684; and/or (f) a CDR-L3 comprising the amino acid sequence set forth SEQ ID NO: 698.

In some embodiments of the pharmaceutical composition of the anti-CD30L antibody or antigen binding fragment provided herein, the antibody or antigen binding fragment thereof comprises:
(i)(a) a CDR-H1 comprising the amino acid sequence set forth in SEQ ID NO: 636; (b) a CDR-H2 comprising the amino acid sequence set forth in SEQ ID NO: 650; (c) a CDR-H3 comprising the amino acid sequence set forth in SEQ ID NO: 664; (d) a CDR-L1 comprising the amino acid sequence set forth in SEQ ID NO: 678; (e) a CDR-L2 comprising the amino acid sequence set forth SEQ ID NO: 692; and/or (f) a CDR-L3 comprising the amino acid sequence set forth SEQ ID NO: 706;
(ii)(a) a CDR-H1 comprising the amino acid sequence set forth in SEQ ID NO: 117; (b) a CDR-H2 comprising the amino acid sequence set forth in SEQ ID NO: 157; (c) a CDR-H3 comprising the amino acid sequence set forth in SEQ ID NO: 197; (d) a CDR-L1 comprising the amino acid sequence set forth in SEQ ID NO: 317; (e) a CDR-L2 comprising the amino acid sequence set forth SEQ ID NO: 357; and/or (f) a CDR-L3 comprising the amino acid sequence set forth SEQ ID NO: 397;
(iii)(a) a CDR-H1 comprising the amino acid sequence set forth in SEQ ID NO: 115; (b) a CDR-H2 comprising the amino acid sequence set forth in SEQ ID NO: 155; (c) a CDR-H3 comprising the amino acid sequence set forth in SEQ ID NO: 195; (d) a CDR-L1 comprising the amino acid sequence set forth in SEQ ID NO: 315; (e) a CDR-L2 comprising the amino acid sequence set forth SEQ ID NO: 355; and/or (f) a CDR-L3 comprising the amino acid sequence set forth SEQ ID NO: 395;
(iv)(a) a CDR-H1 comprising the amino acid sequence set forth in SEQ ID NO: 116; (b) a CDR-H2 comprising the amino acid sequence set forth in SEQ ID NO: 156; (c) a CDR-H3 comprising the amino acid sequence set forth in SEQ ID NO: 196; (d) a CDR-L1 comprising the amino acid sequence set forth in SEQ ID NO: 316; (e) a CDR-L2 comprising the amino acid sequence set forth SEQ ID NO: 356; and/or (f) a CDR-L3 comprising the amino acid sequence set forth SEQ ID NO: 396;
(v)(a) a CDR-H1 comprising the amino acid sequence set forth in SEQ ID NO: 118; (b) a CDR-H2 comprising the amino acid sequence set forth in SEQ ID NO: 158; (c) a CDR-H3 comprising the amino acid sequence set forth in SEQ ID NO: 198; (d) a CDR-L1 comprising the amino acid sequence set forth in SEQ ID NO: 318; (e) a CDR-L2 comprising the amino acid sequence set forth SEQ ID NO: 358; and/or (f) a CDR-L3 comprising the amino acid sequence set forth SEQ ID NO: 398;
(vi)(a) a CDR-H1 comprising the amino acid sequence set forth in SEQ ID NO: 119; (b) a CDR-H2 comprising the amino acid sequence set forth in SEQ ID NO: 159; (c) a CDR-H3 comprising the amino acid sequence set forth in SEQ ID NO: 199; (d) a CDR-L1 comprising the amino acid sequence set forth in SEQ ID NO: 319; (e) a CDR-L2 comprising the amino acid sequence set forth SEQ ID NO: 359; and/or (f) a CDR-L3 comprising the amino acid sequence set forth SEQ ID NO: 399; or
(vii)(a) a CDR-H1 comprising the amino acid sequence set forth in SEQ ID NO: 629; (b) a CDR-H2 comprising the amino acid sequence set forth in SEQ ID NO: 643; (c) a CDR-H3 comprising the amino acid sequence set forth in SEQ ID NO: 657; (d) a CDR-L1 comprising the amino acid sequence set forth in SEQ ID NO: 671; (e) a CDR-L2 comprising the amino acid sequence set forth SEQ ID NO: 685; and/or (f) a CDR-L3 comprising the amino acid sequence set forth SEQ ID NO: 699.

In some embodiments of the pharmaceutical composition of the anti-CD30L antibody or antigen binding fragment provided herein, the antibody or antigen binding fragment thereof comprises:
(i)(a) a CDR-H1 comprising the amino acid sequence set forth in SEQ ID NO: 637; (b) a CDR-H2 comprising the amino acid sequence set forth in SEQ ID NO: 651; (c) a CDR-H3 comprising the amino acid sequence set forth in SEQ ID NO: 665; (d) a CDR-L1 comprising the amino acid sequence set forth in SEQ ID NO: 679; (e) a CDR-L2 comprising the amino acid sequence set forth SEQ ID NO: 693; and/or (f) a CDR-L3 comprising the amino acid sequence set forth SEQ ID NO: 707;
(ii)(a) a CDR-H1 comprising the amino acid sequence set forth in SEQ ID NO: 482; (b) a CDR-H2 comprising the amino acid sequence set forth in SEQ ID NO: 520; (c) a CDR-H3 comprising the amino acid sequence set forth in SEQ ID NO: 545; (d) a CDR-L1 comprising the amino acid sequence set forth in SEQ ID NO: 570; (e) a CDR-L2 comprising the amino acid sequence set forth SEQ ID NO: 595; and/or (f) a CDR-L3 comprising the amino acid sequence set forth SEQ ID NO: 620;
(iii)(a) a CDR-H1 comprising the amino acid sequence set forth in SEQ ID NO: 480; (b) a CDR-H2 comprising the amino acid sequence set forth in SEQ ID NO: 518; (c) a CDR-H3 comprising the amino acid sequence set forth in SEQ ID NO: 543; (d) a CDR-L1 comprising the amino acid sequence set forth in SEQ ID NO: 568; (e) a CDR-L2 comprising the amino acid sequence set forth SEQ ID NO: 593; and/or (f) a CDR-L3 comprising the amino acid sequence set forth SEQ ID NO: 618;
(iv)(a) a CDR-H1 comprising the amino acid sequence set forth in SEQ ID NO: 481; (b) a CDR-H2 comprising the amino acid sequence set forth in SEQ ID NO: 519; (c) a CDR-H3 comprising the amino acid sequence set forth in SEQ ID NO: 544; (d) a CDR-L1 comprising the amino acid sequence set forth in SEQ ID NO: 569; (e) a CDR-L2 comprising the amino acid sequence set forth SEQ ID NO: 594; and/or (f) a CDR-L3 comprising the amino acid sequence set forth SEQ ID NO: 619;
(v)(a) a CDR-H1 comprising the amino acid sequence set forth in SEQ ID NO: 483; (b) a CDR-H2 comprising the amino acid sequence set forth in SEQ ID NO: 521; (c) a CDR-H3 comprising the amino acid sequence set forth in SEQ ID NO: 546; (d) a CDR-L1 comprising the amino acid sequence set forth in SEQ ID NO: 571; (e) a CDR-L2 comprising the amino acid sequence set forth SEQ ID NO: 596; and/or (f) a CDR-L3 comprising the amino acid sequence set forth SEQ ID NO: 621;
(vi)(a) a CDR-H1 comprising the amino acid sequence set forth in SEQ ID NO: 484; (b) a CDR-H2 comprising the amino acid sequence set forth in SEQ ID NO: 522; (c) a CDR-H3 comprising the amino acid sequence set forth in SEQ ID NO: 547; (d) a CDR-L1 comprising the amino acid sequence set forth in SEQ ID NO: 572; (e) a CDR-L2 comprising the amino acid sequence set forth SEQ ID NO: 597; and/or (f) a CDR-L3 comprising the amino acid sequence set forth SEQ ID NO: 622; or
(vii)(a) a CDR-H1 comprising the amino acid sequence set forth in SEQ ID NO: 630; (b) a CDR-H2 comprising the amino acid sequence set forth in SEQ ID NO: 644; (c) a CDR-H3 comprising the amino acid sequence set forth in SEQ ID NO: 658; (d) a CDR-L1 comprising the amino acid sequence set forth in SEQ ID NO: 672; (e) a CDR-L2 comprising the amino acid sequence set forth SEQ ID NO: 686; and/or (f) a CDR-L3 comprising the amino acid sequence set forth SEQ ID NO: 700.

In some embodiments of the pharmaceutical composition of the anti-CD30L antibody or antigen binding fragment provided herein, the antibody or antigen binding fragment thereof comprises:
(i)(a) a CDR-H1 comprising the amino acid sequence set forth in SEQ ID NO: 638; (b) a CDR-H2 comprising the amino acid sequence set forth in SEQ ID NO: 652; (c) a CDR-H3 comprising the amino acid sequence set forth in SEQ ID NO: 666; (d) a CDR-L1 comprising the amino acid sequence set forth in SEQ ID NO: 680; (e) a CDR-L2 comprising the amino acid sequence set forth SEQ ID NO: 694; and/or (f) a CDR-L3 comprising the amino acid sequence set forth SEQ ID NO: 708;
(ii)(a) a CDR-H1 comprising the amino acid sequence set forth in SEQ ID NO: 467; (b) a CDR-H2 comprising the amino acid sequence set forth in SEQ ID NO: 492; (c) a CDR-H3 comprising the amino acid sequence set forth in SEQ ID NO: 530; (d) a CDR-L1 comprising the amino acid sequence set forth in SEQ ID NO: 555; (e) a CDR-L2 comprising the amino acid sequence set forth SEQ ID NO: 580; and/or (f) a CDR-L3 comprising the amino acid sequence set forth SEQ ID NO: 605;
(iii)(a) a CDR-H1 comprising the amino acid sequence set forth in SEQ ID NO: 465; (b) a CDR-H2 comprising the amino acid sequence set forth in SEQ ID NO: 490; (c) a CDR-H3 comprising the amino acid sequence set forth in SEQ ID NO: 528; (d) a CDR-L1 comprising the amino acid sequence set forth in SEQ ID NO: 553; (e) a CDR-L2 comprising the amino acid sequence set forth SEQ ID NO: 578; and/or (f) a CDR-L3 comprising the amino acid sequence set forth SEQ ID NO: 603;
(iv)(a) a CDR-H1 comprising the amino acid sequence set forth in SEQ ID NO: 466; (b) a CDR-H2 comprising the amino acid sequence set forth in SEQ ID NO: 491; (c) a CDR-H3 comprising the amino acid sequence set forth in SEQ ID NO: 529; (d) a CDR-L1 comprising the amino acid sequence set forth in SEQ ID NO: 554; (e) a CDR-L2 comprising the amino acid sequence set forth SEQ ID NO: 579; and/or (f) a CDR-L3 comprising the amino acid sequence set forth SEQ ID NO: 604;
(v)(a) a CDR-H1 comprising the amino acid sequence set forth in SEQ ID NO: 468; (b) a CDR-H2 comprising the amino acid sequence set forth in SEQ ID NO: 493; (c) a CDR-H3 comprising the amino acid sequence set forth in SEQ ID NO: 531; (d) a CDR-L1 comprising the amino acid sequence set forth in SEQ ID NO: 556; (e) a CDR-L2 comprising the amino acid sequence set forth SEQ ID NO: 581; and/or (f) a CDR-L3 comprising the amino acid sequence set forth SEQ ID NO: 606;
(vi)(a) a CDR-H1 comprising the amino acid sequence set forth in SEQ ID NO: 469; (b) a CDR-H2 comprising the amino acid sequence set forth in SEQ ID NO: 494; (c) a CDR-H3 comprising the amino acid sequence set forth in SEQ ID NO: 532; (d) a CDR-L1 comprising the amino acid sequence set forth in SEQ ID NO: 557; (e) a CDR-L2 comprising the amino acid sequence set forth SEQ ID NO: 582; and/or (f) a CDR-L3 comprising the amino acid sequence set forth SEQ ID NO: 607; or
(vii)(a) a CDR-H1 comprising the amino acid sequence set forth in SEQ ID NO: 631; (b) a CDR-H2 comprising the amino acid sequence set forth in SEQ ID NO: 645; (c) a CDR-H3 comprising the amino acid sequence set forth in SEQ ID NO: 659; (d) a CDR-L1 comprising the amino acid sequence set forth in SEQ ID NO: 673; (e) a CDR-L2 comprising the amino acid sequence set forth SEQ ID NO: 687; and/or (f) a CDR-L3 comprising the amino acid sequence set forth SEQ ID NO: 701.

In some embodiments of the pharmaceutical composition of the anti-CD30L antibody or antigen binding fragment provided herein, the antibody or antigen binding fragment thereof comprises:
(i)(a) a CDR-H1 comprising the amino acid sequence set forth in SEQ ID NO: 639; (b) a CDR-H2 comprising the amino acid sequence set forth in SEQ ID NO: 653; (c) a CDR-H3 comprising the amino acid sequence set forth in SEQ ID NO: 667; (d) a CDR-L1 comprising the amino acid sequence set forth in SEQ ID NO: 681; (e) a CDR-L2 comprising the amino acid sequence set forth SEQ ID NO: 695; and/or (f) a CDR-L3 comprising the amino acid sequence set forth SEQ ID NO: 709;
(ii)(a) a CDR-H1 comprising the amino acid sequence set forth in SEQ ID NO: 472; (b) a CDR-H2 comprising the amino acid sequence set forth in SEQ ID NO: 497; (c) a CDR-H3 comprising the amino acid sequence set forth in SEQ ID NO: 535; (d) a CDR-L1 comprising the amino acid sequence set forth in SEQ ID NO: 560; (e) a CDR-L2 comprising the amino acid sequence set forth SEQ ID NO: 585; and/or (f) a CDR-L3 comprising the amino acid sequence set forth SEQ ID NO: 610;
(iii)(a) a CDR-H1 comprising the amino acid sequence set forth in SEQ ID NO: 470; (b) a CDR-H2 comprising the amino acid sequence set forth in SEQ ID NO: 495; (c) a CDR-H3 comprising the amino acid sequence set forth in SEQ ID NO: 533; (d) a CDR-L1 comprising the amino acid sequence set forth in SEQ ID NO: 558; (e) a CDR-L2 comprising the amino acid sequence set forth SEQ ID NO: 583; and/or (f) a CDR-L3 comprising the amino acid sequence set forth SEQ ID NO: 608;
(iv)(a) a CDR-H1 comprising the amino acid sequence set forth in SEQ ID NO: 471; (b) a CDR-H2 comprising the amino acid sequence set forth in SEQ ID NO: 496; (c) a CDR-H3 comprising the amino acid sequence set forth in SEQ ID NO: 534; (d) a CDR-L1 comprising the amino acid sequence set forth in SEQ ID NO: 559; (e) a CDR-L2 comprising the amino acid sequence set forth SEQ ID NO: 584; and/or (f) a CDR-L3 comprising the amino acid sequence set forth SEQ ID NO: 609;
(v)(a) a CDR-H1 comprising the amino acid sequence set forth in SEQ ID NO: 473; (b) a CDR-H2 comprising the amino acid sequence set forth in SEQ ID NO: 498; (c) a CDR-H3 comprising the amino acid sequence set forth in SEQ ID NO: 536; (d) a CDR-L1 comprising the amino acid sequence set forth in SEQ ID NO: 561; (e) a CDR-L2 comprising the amino acid sequence set forth SEQ ID NO: 586; and/or (f) a CDR-L3 comprising the amino acid sequence set forth SEQ ID NO: 611;
(vi)(a) a CDR-H1 comprising the amino acid sequence set forth in SEQ ID NO: 474; (b) a CDR-H2 comprising the amino acid sequence set forth in SEQ ID NO: 499; (c) a CDR-H3 comprising the amino acid sequence set forth in SEQ ID NO: 537; (d) a CDR-L1 comprising the amino acid sequence set forth in SEQ ID NO: 562; (e) a CDR-L2 comprising the amino acid sequence set forth SEQ ID NO: 587; and/or (f) a CDR-L3 comprising the amino acid sequence set forth SEQ ID NO: 612; or
(vii)(a) a CDR-H1 comprising the amino acid sequence set forth in SEQ ID NO: 632; (b) a CDR-H2 comprising the amino acid sequence set forth in SEQ ID NO: 646; (c) a CDR-H3 comprising the amino acid sequence set forth in SEQ ID NO: 660; (d) a CDR-L1 comprising the amino acid sequence set forth in SEQ ID NO: 674; (e) a CDR-L2 comprising the amino acid sequence set forth SEQ ID NO: 688; and/or (f) a CDR-L3 comprising the amino acid sequence set forth SEQ ID NO: 702.

In some embodiments of the pharmaceutical composition of the anti-CD30L antibody or antigen binding fragment provided herein, the antibody or antigen binding fragment thereof comprises:
(i)(a) a CDR-H1 comprising the amino acid sequence set forth in SEQ ID NO: 640; (b) a CDR-H2 comprising the amino acid sequence set forth in SEQ ID NO: 654; (c) a CDR-H3 comprising the amino acid sequence set forth in SEQ ID NO: 668; (d) a CDR-L1 comprising the amino acid sequence set forth in SEQ ID NO: 682; (e) a CDR-L2 comprising the amino acid sequence set forth SEQ ID NO: 696; and/or (f) a CDR-L3 comprising the amino acid sequence set forth SEQ ID NO: 710;
(ii)(a) a CDR-H1 comprising the amino acid sequence set forth in SEQ ID NO: 477; (b) a CDR-H2 comprising the amino acid sequence set forth in SEQ ID NO: 515; (c) a CDR-H3 comprising the amino acid sequence set forth in SEQ ID NO: 540; (d) a CDR-L1 comprising the amino acid sequence set forth in SEQ ID NO: 565; (e) a CDR-L2 comprising the amino acid sequence set forth SEQ ID NO: 590; and/or (f) a CDR-L3 comprising the amino acid sequence set forth SEQ ID NO: 615;
(iii)(a) a CDR-H1 comprising the amino acid sequence set forth in SEQ ID NO: 475; (b) a CDR-H2 comprising the amino acid sequence set forth in SEQ ID NO: 513; (c) a CDR-H3 comprising the amino acid sequence set forth in SEQ ID NO: 538; (d) a CDR-L1 comprising the amino acid sequence set forth in SEQ ID NO: 563; (e) a CDR-L2 comprising the amino acid sequence set forth SEQ ID NO: 588; and/or (f) a CDR-L3 comprising the amino acid sequence set forth SEQ ID NO: 613;
(iv)(a) a CDR-H1 comprising the amino acid sequence set forth in SEQ ID NO: 476; (b) a CDR-H2 comprising the amino acid sequence set forth in SEQ ID NO: 514; (c) a CDR-H3 comprising the amino acid sequence set forth in SEQ ID NO: 539; (d) a CDR-L1 comprising the amino acid sequence set forth in SEQ ID NO: 564; (e) a CDR-L2 comprising the amino acid sequence set forth SEQ ID NO: 589; and/or (f) a CDR-L3 comprising the amino acid sequence set forth SEQ ID NO: 614;
(v)(a) a CDR-H1 comprising the amino acid sequence set forth in SEQ ID NO: 478; (b) a CDR-H2 comprising the amino acid sequence set forth in SEQ ID NO: 516; (c) a CDR-H3 comprising the amino acid sequence set forth in SEQ ID NO: 541; (d) a CDR-L1 comprising the amino acid sequence set forth in SEQ ID NO: 566; (e) a CDR-L2 comprising the amino acid sequence set forth SEQ ID NO: 591; and/or (f) a CDR-L3 comprising the amino acid sequence set forth SEQ ID NO: 616;
(vi)(a) a CDR-H1 comprising the amino acid sequence set forth in SEQ ID NO: 479; (b) a CDR-H2 comprising the amino acid sequence set forth in SEQ ID NO: 517; (c) a CDR-H3 comprising the amino acid sequence set forth in SEQ ID NO: 542; (d) a CDR-L1 comprising the amino acid sequence set forth in SEQ ID NO: 567; (e) a CDR-L2 comprising the amino acid sequence set forth SEQ ID NO: 592; and/or (f) a CDR-L3 comprising the amino acid sequence set forth SEQ ID NO: 617; or
(vii)(a) a CDR-H1 comprising the amino acid sequence set forth in SEQ ID NO: 633; (b) a CDR-H2 comprising the amino acid sequence set forth in SEQ ID NO: 647; (c) a CDR-H3 comprising the amino acid sequence set forth in SEQ ID NO: 661; (d) a CDR-L1 comprising the amino acid sequence set forth in SEQ ID NO: 675; (e) a CDR-L2 comprising the amino acid sequence set forth SEQ ID NO: 689; and/or (f) a CDR-L3 comprising the amino acid sequence set forth SEQ ID NO: 703.

In some embodiments of the pharmaceutical composition of the anti-CD30L antibody or antigen binding fragment provided herein, the antibody or antigen binding fragment thereof comprises:
(i)(a) a CDR-H1 comprising the amino acid sequence set forth in SEQ ID NO: 641; (b) a CDR-H2 comprising the amino acid sequence set forth in SEQ ID NO: 655; (c) a CDR-H3 comprising the amino acid sequence set forth in SEQ ID NO: 669; (d) a CDR-L1 comprising the amino acid sequence set forth in SEQ ID NO: 683; (e) a CDR-L2 comprising the amino acid sequence set forth SEQ ID NO: 697; and/or (f) a CDR-L3 comprising the amino acid sequence set forth SEQ ID NO: 711;
(ii)(a) a CDR-H1 comprising the amino acid sequence set forth in SEQ ID NO: 487; (b) a CDR-H2 comprising the amino acid sequence set forth in SEQ ID NO: 525; (c) a CDR-H3 comprising the amino acid sequence set forth in SEQ ID NO: 550; (d) a CDR-L1 comprising the amino acid sequence set forth in SEQ ID NO: 575; (e) a CDR-L2 comprising the amino acid sequence set forth SEQ ID NO: 600; and/or (f) a CDR-L3 comprising the amino acid sequence set forth SEQ ID NO: 625;
(iii)(a) a CDR-H1 comprising the amino acid sequence set forth in SEQ ID NO: 485; (b) a CDR-H2 comprising the amino acid sequence set forth in SEQ ID NO: 523; (c) a CDR-H3 comprising the amino acid sequence set forth in SEQ ID NO: 548; (d) a CDR-L1 comprising the amino acid sequence set forth in SEQ ID NO: 573; (e) a CDR-L2 comprising the amino acid sequence set forth SEQ ID NO: 598; and/or (f) a CDR-L3 comprising the amino acid sequence set forth SEQ ID NO: 623;
(iv)(a) a CDR-H1 comprising the amino acid sequence set forth in SEQ ID NO: 486; (b) a CDR-H2 comprising the amino acid sequence set forth in SEQ ID NO: 524; (c) a CDR-H3 comprising the amino acid sequence set forth in SEQ ID NO: 549; (d) a CDR-L1 comprising the amino acid sequence set forth in SEQ ID NO: 574; (e) a CDR-L2 comprising the amino acid sequence set forth SEQ ID NO: 599; and/or (f) a CDR-L3 comprising the amino acid sequence set forth SEQ ID NO: 624;
(v)(a) a CDR-H1 comprising the amino acid sequence set forth in SEQ ID NO: 488; (b) a CDR-H2 comprising the amino acid sequence set forth in SEQ ID NO: 526; (c) a CDR-H3 comprising the amino acid sequence set forth in SEQ ID NO: 551; (d) a CDR-L1 comprising the amino acid sequence set forth in SEQ ID NO: 576; (e) a CDR-L2 comprising the amino acid sequence set forth SEQ ID NO: 601; and/or (f) a CDR-L3 comprising the amino acid sequence set forth SEQ ID NO: 626;
(vi)(a) a CDR-H1 comprising the amino acid sequence set forth in SEQ ID NO: 489; (b) a CDR-H2 comprising the amino acid sequence set forth in SEQ ID NO: 527; (c) a CDR-H3 comprising the amino acid sequence set forth in SEQ ID NO: 552; (d) a CDR-L1 comprising the amino acid sequence set forth in SEQ ID NO: 577; (e) a CDR-L2 comprising the amino acid sequence set forth SEQ ID NO: 602; and/or (f) a CDR-L3 comprising the amino acid sequence set forth SEQ ID NO: 627; or
(vii)(a) a CDR-H1 comprising the amino acid sequence set forth in SEQ ID NO: 634; (b) a CDR-H2 comprising the amino acid sequence set forth in SEQ ID NO: 648; (c) a CDR-H3 comprising the amino acid sequence set forth in SEQ ID NO: 662; (d) a CDR-L1 comprising the amino acid sequence set forth in SEQ ID NO: 676; (e) a CDR-L2 comprising the amino acid sequence set forth SEQ ID NO: 690; and/or (f) a CDR-L3 comprising the amino acid sequence set forth SEQ ID NO: 704.

In some embodiments of the pharmaceutical composition of the anti-CD30L antibody or antigen binding fragment provided herein, the antibody or antigen binding fragment thereof comprises:
(i)(a) an immunoglobulin variable region heavy chain (VH) comprising an amino acid sequence having at least about 90, 95, 97, 98, 99, or 100% sequence identity to any one of SEQ ID NOs: 1, 2, 5, 6, 9, 10, 13, 14, 17, 19, 21, 23, 25, 27, 29, and 31; and/or (b) an immunoglobulin variable region light chain (VL) comprising an amino acid sequence having at least about 90, 95, 97, 98, 99, or 100% sequence identity to any one of SEQ ID NOs: 3, 4, 7, 8, 11, 12, 15, 16, 18, 20, 22, 24, 26, 28, and 30;
(ii)(a) a VH comprising an amino acid sequence having at least about 90, 95, 97, 98, 99, or 100% sequence identity to SEQ ID NO: 1; and/or (b) a VL comprising an amino acid sequence having at least about 90, 95, 97, 98, 99, or 100% sequence identity to SEQ ID NO: 3;
(iii)(a) a VH comprising an amino acid sequence having at least about 90, 95, 97, 98, 99, or 100% sequence identity to SEQ ID NO: 2; and/or (b) a VL comprising an amino acid sequence having at least about 90, 95, 97, 98, 99, or 100% sequence identity to SEQ ID NO: 4;
(iv)(a) a VH comprising an amino acid sequence having at least about 90, 95, 97, 98, 99, or 100% sequence identity to SEQ ID NO: 5; and/or (b) a VL comprising an amino acid sequence having at least about 90, 95, 97, 98, 99, or 100% sequence identity to SEQ ID NO: 7;
(v)(a) a VH comprising an amino acid sequence having at least about 90, 95, 97, 98, 99, or 100% sequence identity to SEQ ID NO: 6; and/or (b) a VL comprising an amino acid sequence having at least about 90, 95, 97, 98, 99, or 100% sequence identity to SEQ ID NO: 8;
(vi)(a) a VH comprising an amino acid sequence having at least about 90, 95, 97, 98, 99, or 100% sequence identity to SEQ ID NO: 9; and/or (b) a VL comprising an amino acid sequence having at least about 90, 95, 97, 98, 99, or 100% sequence identity to SEQ ID NO: 11;
(vii)(a) a VH comprising an amino acid sequence having at least about 90, 95, 97, 98, 99, or 100% sequence identity to SEQ ID NO: 10; and/or (b) a VL comprising an amino acid sequence having at least about 90, 95, 97, 98, 99, or 100% sequence identity to SEQ ID NO: 12;
(viii)(a) a VH comprising an amino acid sequence having at least about 90, 95, 97, 98, 99, or 100% sequence identity to SEQ ID NO: 13; and/or (b) a VL comprising an amino acid sequence having at least about 90, 95, 97, 98, 99, or 100% sequence identity to SEQ ID NO: 15;
(ix)(a) a VH comprising an amino acid sequence having at least about 90, 95, 97, 98, 99, or 100% sequence identity to SEQ ID NO: 14; and/or (b) a VL comprising an amino acid sequence having at least about 90, 95, 97, 98, 99, or 100% sequence identity to SEQ ID NO: 16;
(x)(a) a VH comprising an amino acid sequence having at least about 90, 95, 97, 98, 99, or 100% sequence identity to SEQ ID NO: 23; and/or (b) a VL comprising an amino acid sequence having at least about 90, 95, 97, 98, 99, or 100% sequence identity to SEQ ID NO: 24;
(xi)(a) a VH comprising an amino acid sequence having at least about 90, 95, 97, 98, 99, or 100% sequence identity to SEQ ID NO: 25; and/or (b) a VL comprising an amino acid sequence having at least about 90, 95, 97, 98, 99, or 100% sequence identity to SEQ ID NO: 26;
(xii)(a) a VH comprising an amino acid sequence having at least about 90, 95, 97, 98, 99, or 100% sequence identity to SEQ ID NO: 27; and/or (b) a VL comprising an amino acid sequence having at least about 90, 95, 97, 98, 99, or 100% sequence identity to SEQ ID NO: 28;
(xiii)(a) a VH comprising an amino acid sequence having at least about 90, 95, 97, 98, 99, or 100% sequence identity to SEQ ID NO: 29; and/or (b) a VL comprising an amino acid sequence having at least about 90, 95, 97, 98, 99, or 100% sequence identity to SEQ ID NO: 30; or
(xiv)(a) a VH comprising an amino acid sequence having at least about 90, 95, 97, 98, 99, or 100% sequence identity to SEQ ID NO: 31; and/or (b) a VL comprising an amino acid sequence having at least about 90, 95, 97, 98, 99, or 100% sequence identity to SEQ ID NO: 32.

In some embodiments of the pharmaceutical composition of the anti-CD30L antibody or antigen binding fragment provided herein, the antibody or antigen binding fragment thereof comprises:
(i)(a) a VH comprising an amino acid sequence set forth in any one of SEQ ID NOs: 1, 2, 5, 6, 9, 10, 13, 14, 17, 19, 21, 23, 25, 27, 29, and 31; and/or (b) a VL comprising an amino acid sequence set forth in any one of SEQ ID NOs: 3, 4, 7, 8, 11, 12, 15, 16, 18, 20, 22, 24, 26, 28, and 30;
(ii)(a) a VH comprising an amino acid sequence set forth in SEQ ID NO: 1; and/or (b) a VL comprising an amino acid sequence set forth in SEQ ID NO: 3;
(iii)(a) a VH comprising an amino acid sequence set forth in SEQ ID NO: 2; and/or (b) a VL comprising an amino acid sequence set forth in SEQ ID NO: 4;
(iv)(a) a VH comprising an amino acid sequence set forth in SEQ ID NO: 5; and/or (b) a VL comprising an amino acid sequence set forth in SEQ ID NO: 7;
(v)(a) a VH comprising an amino acid sequence set forth in SEQ ID NO: 6; and/or (b) a VL comprising an amino acid sequence set forth in SEQ ID NO: 8;
(vi)(a) a VH comprising an amino acid sequence set forth in SEQ ID NO: 9; and/or (b) a VL comprising an amino acid sequence set forth in SEQ ID NO: 11;
(vii)(a) a VH comprising an amino acid sequence set forth in SEQ ID NO: 10; and/or (b) a VL comprising an amino acid sequence set forth in SEQ ID NO: 12;
(viii)(a) a VH comprising an amino acid sequence set forth in SEQ ID NO: 13; and/or (b) a VL comprising an amino acid sequence set forth in SEQ ID NO: 15;
(ix)(a) a VH comprising an amino acid sequence set forth in SEQ ID NO: 14; and/or (b) a VL comprising an amino acid sequence set forth in SEQ ID NO: 16;
(x)(a) a VH comprising an amino acid sequence set forth in SEQ ID NO: 23; and/or (b) a VL comprising an amino acid sequence set forth in SEQ ID NO: 24;
(xi)(a) a VH comprising an amino acid sequence set forth in SEQ ID NO: 25; and/or (b) a VL comprising an amino acid sequence set forth in SEQ ID NO: 26;
(xii)(a) a VH comprising an amino acid sequence set forth in SEQ ID NO: 27; and/or (b) a VL comprising an amino acid sequence set forth in SEQ ID NO: 28;
(xiii)(a) a VH comprising an amino acid sequence set forth in SEQ ID NO: 29; and/or (b) a VL comprising an amino acid sequence set forth in SEQ ID NO: 30; or
(xiv)(a) a VH comprising an amino acid sequence set forth in SEQ ID NO: 31; and/or (b) a VL comprising an amino acid sequence set forth in SEQ ID NO: 32.

In some embodiments of the pharmaceutical composition of the anti-CD30L antibody or antigen binding fragment provided herein, the antibody or antigen binding fragment thereof further comprises an IgG constant region.

In some embodiments of the pharmaceutical composition of the anti-CD30L antibody or antigen binding fragment provided herein, the antibody or antigen binding fragment thereof further comprises an IgG constant region having reduced antibody-dependent cell-mediated cytotoxicity (ADCC) function as compared to human IgG and/or reduced complement-dependent cytotoxicity (CDC) as compared to human IgG.

In some embodiments of the pharmaceutical composition of the anti-CD30L antibody or antigen binding fragment provided herein, the constant region of the anti-CD30L antibody or antigen binding fragment comprises an amino acid sequence having 80, 85, 90, 95, 97, 98, 99, or 100% sequence identity to the amino acid sequence set forth by any one of SEQ ID NOs: 500-512.

In some embodiments of the pharmaceutical composition of the anti-CD30L antibody or antigen binding fragment provided herein, the constant region of the anti-CD30L antibody or antigen binding fragment comprises the amino acid sequence set forth by any one of SEQ ID NOs: 500-512.

In some embodiments of the pharmaceutical composition of the anti-CD30L antibody or antigen binding fragment provided herein, the antibody or antigen binding fragment thereof comprises a constant region having an amino acid sequence variant corresponding to (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(mmm), per EU numbering.

In some embodiments of the pharmaceutical composition of the anti-CD30L antibody or antigen binding fragment provided herein, the antibody or antigen binding fragment thereof is an IgG antibody.

In some embodiments of the pharmaceutical composition of the anti-CD30L antibody or antigen binding fragment provided herein, the IgG antibody is IgG1, IgG2, IgG3, or IgG4.

In some embodiments of the pharmaceutical composition of the anti-CD30L antibody or antigen binding fragment provided herein, the antibody or antigen binding fragment thereof is human, chimeric, or humanized.

In some embodiments of the pharmaceutical composition of the anti-CD30L antibody or antigen binding fragment provided herein, the antibody or antigen binding fragment thereof is a Fab, F(ab′)₂, a single-domain antibody, or a single chain variable fragment (scFv).

In some embodiments of the pharmaceutical composition of the anti-CD30L antibody or antigen binding fragment provided herein, the antibody or antigen binding fragment thereof is a bispecific or multispecific antibody.

In some embodiments of the pharmaceutical composition of the anti-CD30L antibody or antigen binding fragment provided herein, the antibody or antigen binding fragment thereof binds one or more amino acids residues of CD30L that interact with CD30.

In some embodiments of the pharmaceutical composition of the anti-CD30L antibody or antigen binding fragment provided herein, the antibody or antigen binding fragment thereof inhibits a binding interaction between CD30L and CD30.

In some embodiments of the pharmaceutical composition of the anti-CD30L antibody or antigen binding fragment provided herein, the antibody or antigen binding fragment thereof blocks a binding interaction between CD30L and CD30.

In some embodiments of the pharmaceutical composition of the anti-CD30L antibody or antigen binding fragment provided herein, the inhibition or blocking is determined in an ELISA assay, a cell binding assay with CD30L expressing cells, or a surface plasmon resonance (SPR) assay.

In some embodiments of the pharmaceutical composition of the anti-CD30L antibody or antigen binding fragment provided herein, the antibody or antigen binding fragment thereof specifically binds to CD30L.

In some embodiments of the pharmaceutical composition of the anti-CD30L antibody or antigen binding fragment provided herein, the antibody or antigen binding fragment thereof (i) inhibits interleukin-8 secretion in a cell-based assay, (ii) inhibits interleukin-6 secretion in a cell-based assay, or (iii) both (i) and (ii).

In some embodiments of the pharmaceutical composition of the anti-CD30L antibody or antigen binding fragment provided herein, the antibody or antigen binding fragment thereof (i) blocks interleukin-8 secretion in a cell-based assay, (ii) blocks interleukin-6 secretion in a cell-based assay, or (iii) both (i) and (ii).

In some embodiments of the pharmaceutical composition of the anti-CD30L antibody or antigen binding fragment provided herein, the cell-based assay is a dual cell assay with a cell expressing CD30 and a cell expressing CD30L.

In some embodiments of the pharmaceutical composition of the anti-CD30L antibody or antigen binding fragment provided herein, the antibody or antigen binding fragment thereof binds to (i) human CD30L, (ii) cynomolgus CD30L, or (iii) both human CD30L and cynomolgus CD30L.

In some embodiments of the pharmaceutical composition of the anti-CD30L antibody or antigen binding fragment provided herein, the antibody or antigen binding fragment thereof binds to CD30L with a dissociation equilibrium constant (K_{D}) of no more than 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, or 1000 pM.

In some embodiments of the pharmaceutical composition of the anti-CD30L antibody or antigen binding fragment provided herein, the antibody or antigen binding fragment thereof binds to CD30L with an association rate constant (kₒₙ) of at least 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 1.1×10⁶, 1.2×10⁶, 1.3×10⁶, 1.4×10⁶, 1.5×10⁶, or 1.55×10⁶M⁻¹ S⁻¹.

In some embodiments of the pharmaceutical composition of the anti-CD30L antibody or antigen binding fragment provided herein, the antibody or antigen binding fragment thereof binds to CD30L with a dissociation rate constant (k_{off}) of no more than 1.4×10⁻⁴, 1.41×10⁻⁴, 1.5×10⁻⁴, 1.6×10⁻⁴, 1.7×10⁻⁴, 1.8×10⁻⁴, 1.9×10⁻⁴, 2.0×10⁻⁴, 2.1×10⁻⁴, 2.2×10⁻⁴, 2.3×10⁻⁴, 2.4×10⁻⁴, 2.5×10⁻⁴, 2.6×10⁻⁴, 2.7×10⁻⁴, 2.8×10⁻⁴, 2.9×10⁻⁴, 3.0×10⁻⁴, 3.1×10⁻⁴, 3.2×10⁻⁴, 3.3×10⁻⁴, 3.4×10⁻⁴, or 3.5×10⁻⁴ S⁻¹.

In one aspect, provided herein is a pharmaceutical composition comprising an anti-CD30L antibody or antigen binding fragment thereof, wherein the antibody or antigen binding fragment thereof binds isoform 1 of CD30L, and wherein the antibody or antigen binding fragment thereof does not bind to isoform 2 of CD30L. In some embodiments, the isoform 1 of CD30L comprises the amino acid sequence set forth in SEQ ID NO: 34 and wherein the isoform 2 of CD30L comprises the amino acid sequence set forth in SEQ ID NO: 35.

In some embodiments of the pharmaceutical composition of the anti-CD30L antibody or antigen binding fragment provided herein, the antibody or antigen binding fragment thereof binds isoform 1 of CD30L, and wherein the antibody or antigen binding fragment thereof does not bind to isoform 2 of CD30L. In some embodiments, the isoform 1 of CD30L comprises the amino acid sequence set forth in SEQ ID NO: 34 and wherein the isoform 2 of CD30L comprises the amino acid sequence set forth in SEQ ID NO: 35.

In some embodiments of the pharmaceutical composition of the anti-CD30L antibody or antigen binding fragment provided herein, the antibody or antigen binding fragment thereof is recombinant antibody or antigen binding fragment thereof.

In some embodiments of the pharmaceutical composition of the anti-CD30L antibody or antigen binding fragment provided herein, the antibody or antigen binding fragment thereof is isolated antibody or antigen binding fragment thereof.

In some embodiments of the pharmaceutical composition of the anti-CD30L antibody or antigen binding fragment provided herein, the anti-CD30L antibody or antigen binding fragment is
(a) at a concentration (i) from about 50 mg/ml to about 275 mg/ml, (ii) from about 50 mg/ml to about 250 mg/ml, (iii) from about 50 mg/ml to about 225 mg/ml, (iv) from about 50 mg/ml to about 200 mg/ml, (v) from about 50 mg/ml to about 175 mg/ml, (vi) from about 50 mg/ml to about 150 mg/ml, (vii) from about 50 mg/ml to about 125 mg/ml, (viii) from about 50 mg/ml to about 100 mg/ml, or (ix) from about 50 mg/ml to about 75 mg/ml;
(b) at a concentration (i) from about 75 mg/ml to about 275 mg/ml, (ii) from about 75 mg/ml to about 250 mg/ml, (iii) from about 75 mg/ml to about 225 mg/ml, (iv) from about 75 mg/ml to about 200 mg/ml, (v) from about 75 mg/ml to about 175 mg/ml, (vi) from about 75 mg/ml to about 150 mg/ml, (vii) from about 75 mg/ml to about 125 mg/ml, or (viii) from about 75 mg/ml to about 100 mg/ml;
(c) at a concentration (i) from about 100 mg/ml to about 275 mg/ml, (ii) from about 100 mg/ml to about 250 mg/ml, (iii) from about 100 mg/ml to about 225 mg/ml, (iv) from about 100 mg/ml to about 200 mg/ml, (v) from about 100 mg/ml to about 175 mg/ml, (vi) from about 100 mg/ml to about 150 mg/ml, or (vii) from about 100 mg/ml to about 125 mg/ml;
(d) at a concentration (i) from about 125 mg/ml to about 275 mg/ml, (ii) from about 125 mg/ml to about 250 mg/ml, (iii) from about 125 mg/ml to about 225 mg/ml, (iv) from about 125 mg/ml to about 200 mg/ml, (v) from about 125 mg/ml to about 175 mg/ml, or (vi) from about 125 mg/ml to about 150 mg/ml;
(e) at a concentration (i) from about 150 mg/ml to about 275 mg/ml, (ii) from about 150 mg/ml to about 250 mg/ml, (iii) from about 150 mg/ml to about 225 mg/ml, (iv) from about 150 mg/ml to about 200 mg/ml, or (v) from about 150 mg/ml to about 175 mg/ml;
(f) at a concentration (i) from about 175 mg/ml to about 275 mg/ml, (ii) from about 175 mg/ml to about 250 mg/ml, (iii) from about 175 mg/ml to about 225 mg/ml, or (iv) from about 175 mg/ml to about 200 mg/ml;
(g) at a concentration (i) from about 200 mg/ml to about 275 mg/ml, (ii) from about 200 mg/ml to about 250 mg/ml, or (iii) from about 200 mg/ml to about 225 mg/ml; or
(h) at a concentration (i) at least about 50 mg/mL, (ii) at least about 75 mg/ml, (iii) at least about 100 mg/ml, (iv) at least about 125 mg/ml, (v) at least about 150 mg/ml, (vi) at least about 175 mg/ml, (vii) at least about 200 mg/ml, (viii) at least about 225 mg/ml, or (ix) at least about 250 mg/ml.

In one aspect, provided herein is a pharmaceutical composition comprising an antibody or antigen binding fragment that binds to CD30L (anti-CD30L antibody or antigen binding fragment) at a concentration (i) at least about 50 mg/mL, (ii) at least about 75 mg/ml, (iii) at least about 100 mg/ml, (iv) at least about 125 mg/ml, (v) at least about 150 mg/ml, (vi) at least about 175 mg/ml, (vii) at least about 200 mg/ml, (viii) at least about 225 mg/ml, or (ix) at least about 250 mg/ml.

In another aspect, provided herein is a pharmaceutical composition comprising an antibody or antigen binding fragment that binds to CD30L (anti-CD30L antibody or antigen binding fragment)
(a) at a concentration (i) from about 50 mg/ml to about 275 mg/ml, (ii) from about 50 mg/ml to about 250 mg/ml, (iii) from about 50 mg/ml to about 225 mg/ml, (iv) from about 50 mg/ml to about 200 mg/ml, (v) from about 50 mg/ml to about 175 mg/ml, (vi) from about 50 mg/ml to about 150 mg/ml, (vii) from about 50 mg/ml to about 125 mg/ml, (viii) from about 50 mg/ml to about 100 mg/ml, or (ix) from about 50 mg/ml to about 75 mg/ml;
(b) at a concentration (i) from about 75 mg/ml to about 275 mg/ml, (ii) from about 75 mg/ml to about 250 mg/ml, (iii) from about 75 mg/ml to about 225 mg/ml, (iv) from about 75 mg/ml to about 200 mg/ml, (v) from about 75 mg/ml to about 175 mg/ml, (vi) from about 75 mg/ml to about 150 mg/ml, (vii) from about 75 mg/ml to about 125 mg/ml, or (viii) from about 75 mg/ml to about 100 mg/ml;
(c) at a concentration (i) from about 100 mg/ml to about 275 mg/ml, (ii) from about 100 mg/ml to about 250 mg/ml, (iii) from about 100 mg/ml to about 225 mg/ml, (iv) from about 100 mg/ml to about 200 mg/ml, (v) from about 100 mg/ml to about 175 mg/ml, (vi) from about 100 mg/ml to about 150 mg/ml, or (vii) from about 100 mg/ml to about 125 mg/ml;
(d) at a concentration (i) from about 125 mg/ml to about 275 mg/ml, (ii) from about 125 mg/ml to about 250 mg/ml, (iii) from about 125 mg/ml to about 225 mg/ml, (iv) from about 125 mg/ml to about 200 mg/ml, (v) from about 125 mg/ml to about 175 mg/ml, or (vi) from about 125 mg/ml to about 150 mg/ml;
(e) at a concentration (i) from about 150 mg/ml to about 275 mg/ml, (ii) from about 150 mg/ml to about 250 mg/ml, (iii) from about 150 mg/ml to about 225 mg/ml, (iv) from about 150 mg/ml to about 200 mg/ml, or (v) from about 150 mg/ml to about 175 mg/ml;
(f) at a concentration (i) from about 175 mg/ml to about 275 mg/ml, (ii) from about 175 mg/ml to about 250 mg/ml, (iii) from about 175 mg/ml to about 225 mg/ml, or (iv) from about 175 mg/ml to about 200 mg/ml; or
(g) at a concentration (i) from about 200 mg/ml to about 275 mg/ml, (ii) from about 200 mg/ml to about 250 mg/ml, or (iii) from about 200 mg/ml to about 225 mg/ml.

In some embodiments of the pharmaceutical composition of the anti-CD30L antibody or antigen binding fragment provided herein, the pharmaceutical composition comprises a buffer. In some embodiments, the buffer is selected from the group consisting of an acetate buffer, a succinate buffer, a histidine buffer, a phosphate buffer, and a citrate buffer. In some embodiments, the buffer is a histidine buffer. In some embodiments, the concentration of the buffer is from 0.1 mM to 1 M. In some embodiments, the concentration of the buffer is from 1 mM to 100 mM. In some embodiments, the concentration of the buffer is from 10 mM to 50 mM. In some embodiments, the concentration of the buffer is from about 20 mM. In some embodiments, the pH of the buffer is from 4 to 7. In some embodiments, the pH of the buffer is from 5 to 6. In some embodiments, the pH of the buffer is about 5.5.

In some embodiments of the pharmaceutical composition of the anti-CD30L antibody or antigen binding fragment provided herein, the pharmaceutical composition comprises a pharmaceutically acceptable excipient. In some embodiments, the excipient is arginine, arginine hydrochloride (arginine HCl), proline, a polyol, sodium chloride, glycine, lysine, lysine-hydrochloride, arginine glutamate, potassium chloride, magnesium chloride, calcium chloride, or a combination thereof. In some embodiments, the excipient is arginine HCl. In some embodiments, the arginine HCl is (i) at a concentration from 25 mM to 300 mM, (ii) at a concentration from 50 mM to 250 mM (iii) at a concentration from 50 mM to 200 mM, (iv) at a concentration from 75 mM to 150 mM, or (v) at a concentration of about 100 mM. In some embodiments, the polyol is selected from the group consisting of sugar, sugar alcohol, and sugar acid. In some embodiments, the sugar is sucrose. In some embodiments, the sucrose is: (i) at a concentration from 5% to 10% (weight to volume, w/v), (ii) at a concentration of about 5% (w/v), (iii) at a concentration from 150 mM to 300 mM, or (iv) at a concentration of about 150 mM.

In some embodiments of the pharmaceutical composition of the anti-CD30L antibody or antigen binding fragment provided herein, the pharmaceutical composition comprises a surfactant. In some embodiments, the surfactant is a polysorbate. In some embodiments, the surfactant is polysorbate-80 (PS80). In some embodiments, the surfactant is poloxamer 188 (P188). In some embodiments, the surfactant is polysorbate-20 (PS20). In some embodiments, the concentration of the surfactant is (i) from 0.001% to 0.1% (w/v), (ii) from 0.005 to 0.05% (w/v), (iii) from 0.01 to 0.05% (w/v), (iv) from 0.01 to 0.04% (w/v), (v) from 0.01 to 0.03% (w/v), (vi) from 0.01 to 0.02% (w/v), (vii) about 0.02% (w/v), (viii) about 0.03% (w/v), (ix) about 0.04% (w/v), or (x) about 0.05% (w/v).

In some embodiments of the pharmaceutical composition of the anti-CD30L antibody or antigen binding fragment provided herein, the pharmaceutical composition comprises sodium chloride (NaCl). In some embodiments, the concentration of NaCl is (i) from 25 mM to 150 mM, (ii) from 25 mM to 100 mM (iii) from 50 mM to 100 mM, (iv) from 75 mM to 100 mM, or (v) about 50 mM.

In some embodiments of the pharmaceutical composition of the anti-CD30L antibody or antigen binding fragment provided herein, the pharmaceutical composition comprises an anti-oxidant.

In some embodiments of the pharmaceutical composition of the anti-CD30L antibody or antigen binding fragment provided herein, the pharmaceutical composition does not comprise an anti-oxidant.

In some embodiments of the pharmaceutical composition of the anti-CD30L antibody or antigen binding fragment provided herein, the viscosity of the composition is from 1 to 50 cP at 25° C. In some embodiments of the pharmaceutical composition of the anti-CD30L antibody or antigen binding fragment provided herein, the viscosity of the composition is from 10 to 25 cP at 25° C. In some embodiments of the pharmaceutical composition of the anti-CD30L antibody or antigen binding fragment provided herein, the viscosity of the composition is from 1 to 50, 2 to 49, 3 to 48, 4 to 46, 5 to 45, 6 to 44, 7 to 43, 8 to 42, 9 to 41, 10 to 40, 10 to 35, 10 to 30, 11 to 29, 12 to 28, 13 to 27, 14 to 26, 15 to 25, 16 to 24, 17 to 23, 18 to 22, 18 to 21, 18 to 20, 18 to 19, 19 to 21, 1 to 10, 1 to 15, or 1 to 20 cP at 25° C. In some embodiments of the pharmaceutical composition of the anti-CD30L antibody or antigen binding fragment provided herein, the viscosity of the composition is no more than 25 cP, no more than 24 cP, no more than 23 cP, no more than 22 cP, no more than 21 cP, no more than 20 cP, or no more than 19 cP at 25° C. In some embodiments of the pharmaceutical composition of the anti-CD30L antibody or antigen binding fragment provided herein, the viscosity of the composition is about 15 cP, about 16 cP, about 17 cP, about 18 cP, about 19 cP, about 20 cP, about 21 cP, about 22 cP, about 23 cP, about 24 cP, or about 25 cP at 25° C.

In some embodiments of the pharmaceutical composition of the anti-CD30L antibody or antigen binding fragment provided herein, the composition is a stable composition. In some embodiments of the pharmaceutical composition of the anti-CD30L antibody or antigen binding fragment provided herein, the composition is a stable: (i) for at least 2, 4, 8, 12, 16, 20, or 24 weeks when stored at 25° C.±5° C., (ii) for at least 2, 4, or 8 weeks when stored at 40° C.±5° C., or (iii) for at least 2, 4, 8, 12, 16, 20, or 24 weeks when stored at 25° C.±5° C. In some embodiments, the stability of the composition is determined by: (i) maintenance of at least 98% of the antibody main peak in size exclusion chromatography, (ii) maintenance of at least 65% of the antibody main peak in iCIEF, (iii) maintenance of at least 98% purity, (iv) maintenance of at least 99% of the antibody concentration, (v) maintenance of no more than 500 particles/ml of particles over 2 μm, no more than 10 particles/ml of particles over 10 μm, and/or no more than 2 particles/ml of particles over 25 μm, and/or (vi) any combination of (i) to (iv).

In some embodiments of the pharmaceutical composition of the anti-CD30L antibody or antigen binding fragment provided herein, the composition comprises the anti-CD30L antibody or antigen binding fragment at a concentration of 200 mg/mL, 20 mM histidine buffer, 100 mM arginine monohydrochloride, and 0.02% (w/v) PS20, at pH 5.5.

In one aspect, provided herein is a pharmaceutical composition comprising an anti-CD30L antibody or antigen binding fragment at a concentration of 200 mg/mL, 20 mM histidine buffer, 100 mM arginine monohydrochloride, and 0.02% (w/v) PS20, at pH 5.5.

In some embodiments of the pharmaceutical composition of the anti-CD30L antibody or antigen binding fragment provided herein, the anti-CD30L blocks both the soluble CD30L and the transmembrane CD30L.

In another aspect, provided herein is a method of making the pharmaceutical formulation described herein, comprising: culturing a cell of in a medium, wherein the cell comprises one or more polynucleotides comprising nucleotide sequences encoding a heavy chain, a light chain, or both a heavy chain and a light chain of the antibody or antigen-binding fragment thereof. In some embodiments of the method of making the pharmaceutical formulation, the method further comprises expressing the one or more polynucleotides in the cell. In some embodiments of the method of making the pharmaceutical formulation, the method further comprises purifying the antibody or antigen-binding fragment thereof. In some embodiments of the method of making the pharmaceutical formulation, the method further comprises a formulation step.

In some embodiments, the pharmaceutical composition provided herein is for use in a method of inhibiting binding of CD30L to CD30.

In some embodiments, the pharmaceutical composition provided herein is for use in a method of inhibiting activation of CD30 signaling in a cell.

In some embodiments, the pharmaceutical composition provided herein is for use in a method of inhibiting activation, expression, and/or secretion of a pro-inflammatory cytokine protein. In some embodiments, the pro-inflammatory cytokine protein is interleukin-8 and/or interleukin-6. In some embodiments, the interleukin-8 is expressed or released by a T lymphocyte.

In some embodiments, the pharmaceutical composition provided herein is for use in treating an autoimmune disease in an individual in need thereof. In some embodiments, the autoimmune disease is irritable bowel disease. In some embodiments, the irritable bowel disease comprises ulcerative colitis (UC) or Crohn's Disease (CD).

In one aspect, provided herein is a method of treating or ameliorating an autoimmune disease in an individual in need thereof, the method comprising administering to the individual the pharmaceutical composition described herein, thereby treating or ameliorating the autoimmune disease. In some embodiments, the autoimmune disease is irritable bowel disease. In some embodiments, the irritable bowel disease comprises ulcerative colitis (UC) or Crohn's Disease (CD).

In another aspect, provided herein is a method for inhibiting and/or reducing binding of CD30L to CD30 in an individual afflicted with an inflammatory or autoimmune disorder, the method comprising administering to the individual afflicted with the inflammatory or autoimmune disorder the pharmaceutical composition described herein, thereby inhibiting and/or reducing the binding of CD30L to CD30. In some embodiments, the individual is afflicted with an autoimmune disease. In some embodiments, the autoimmune disease is irritable bowel disease. In some embodiments, the irritable bowel disease comprises ulcerative colitis (UC) or Crohn's Disease (CD).

In a further aspect, provided herein is a method of reducing and/or inhibiting inflammation in an individual, the method comprising administering to the individual the pharmaceutical composition described herein, thereby reducing and/or inhibiting inflammation. In some embodiments, reducing and/or inhibiting inflammation comprises reducing an amount of pro-inflammatory cytokine expression or secretion in the individual or a tissue of the individual. In some embodiments, wherein reducing and/or inhibiting inflammation comprises reducing an amount of pro-inflammatory cytokine expression or secretion by a T lymphocyte of the individual. In some embodiments, the pro-inflammatory cytokine comprises interleukin-8. In some embodiments, the individual has an autoimmune disease. In some embodiments, the autoimmune disease is irritable bowel disease. In some embodiments, the irritable bowel disease comprises ulcerative colitis (UC) or Crohn's Disease (CD).

### 3. BRIEF DESCRIPTION OF THE DRAWINGS

FIGS. 1A-1E depict dose dependent curve of various anti-CD30L antibodies in B16-huCD30L cell binding assay (1st left column), inhibition/blocking of IL-8 release in a dual cell assay (B16-huCD30L cells and K299 cells) (2nd column from the left), ELISA assay of binding to rhCD30L (3rd column from the left), and ELISA assay of antibody-mediated blocking of CD30-Fc binding to huCD30L (4th column from the left). Units for ELISA are OD 450 nm; and units for cell binding assays are mean fluorescence intensity (MFI). FIG. 1A: results for clones 2 germlined, 62, 63, 64 and 65; FIG. 1B: results for clones 66, 67, 68, 69, 70 and 71; FIG. 1C: results for clones 72, 73, 74, 75, 76 and 77; FIG. 1D: results for clones 78, 79 80 and 8; FIG. 1E: results for clones 82, 83,84 and 85.

FIG. 2 depicts the overlap mapping of the peptides generated from trypsin, chymotrypsin, ASP-N, elastase, and/or thermolysin proteolysis, demonstrating that the peptide signature covers 94.76% of the CD30L ECD sequence.

FIG. 3 depicts the epitope determined from cross-linking and mass spectrometry studies in the context of parts of the CD30L ECD sequence (SEQ ID NO: 34).

FIG. 4A depicts the cell binding assays for determining anti-CD30L binding to CD30L expressing cells and for determining the blocking/inhibition of CD30 binding to CD30L expressing cells by anti-CD30L. FIG. 4B and FIG. 4C depict representative binding curves for germlined clone 2 from cell binding assay shown in FIG. 4A, with 4B showing binding to human CD30L expressing B16 cells and 4 C showing binding to cynoCD30L expressing HEK293 cells. FIG. 4D and FIG. 4E depict representative blocking curves for germlined clone 2 from cell binding assay shown in FIG. 4A, with 4D showing blocking curves against humanCD30L expressing B16 cells and 4E showing blocking curves against cynoCD30L expressing HEK293 cells. FIG. 4F depicts the ELISA configuration for determining anti-CD30L binding to purified CD30L and FIG. 4G depicts the ELISA configuration for determining the blocking/inhibition of CD30L binding to CD30 by anti-CD30L. FIG. 4H depicts representative binding curve for germlined clone 2 with ELISA assay shown in FIG. 4F and FIG. 4I depicts representative blocking curve for germlined clone 2 with ELISA assay shown in FIG. 4G. FIG. 4J depicts the dual cell IL-8 release assay in which CD30 expressing cells release IL-8 upon signaling by ligation with CD30L expressing cells via the CD30-CD30L interaction. FIG. 4K and FIG. 4L depict representative IL-8 release curves for germlined clone 2 from dual cell assay shown in FIG. 4J, with 4K showing IL-8 release curves with humanCD30L expressing B16 cells and 4L showing IL-8 release curves with cynoCD30L expressing HEK293 cells.

FIG. 5A depicts DSC profile of formulation F1-F12 in the pH/buffer study as described in Section 5.10.4. FIG. 5B depicts PEG precipitation test results in pH/buffer study. FIGS. 5C-5D depict excipient comparison and surfactant comparison of SE-UPLC result in excipients and surfactant strength study. FIGS. 5E-5F depict excipient comparison and surfactant comparison of iCIEF result in excipients and surfactant strength study. FIGS. 5G-5H depict excipient comparison and surfactant comparison of Caliper-SDS-NR result in excipients and surfactant strength study. FIGS. 5I-5J depict excipient comparison and surfactant comparison of Caliper-SDS-R result in excipients and surfactant strength study. FIG. 5K depict DSC profile of formulation Fe01-Fe09 in the excipients and surfactant strength study as described in Section 5.10.5.

FIG. 6A depicts CD30L on top and the main paratope residues of germline-clone-2 anti-CD30L Fab shown in the ribbon representation on the bottom. FIG. 6B depicts CD30L Epitope (left) as listed in Table 77 and germline-clone-2 anti-CD30L Fab Paratope (right) as listed in Table 77. FIG. 6C depicts the overview of the structure of the complex between CD30L and germlined-clone-2 Fab. In the structure, two Fab molecules of germlined clone 2 depicted in light grey bind to two monomers of sCD30L, which two monomer form a CD30L dimer. FIG. 6D and FIG. 6E show that cross-links identified by XL-MS between CD30L and germlined clone 2 Fab are within the expected distance range around the periphery of the non-covalent interactions in antibody-antigen interface found in the Cryo-EM structure, thereby confirming the binding interface identified in the cryoEM structure. FIG. 6F shows the close-up view of the contact site between E109 of CDR-H3 and N165 of CD30L in the cryoEM structure. FIG. 6G shows the close-up view of the contact site between H32 of CDR-L1 and K169 of CD30L in the cryoEM structure. FIG. 6H shows the close-up view of the contact site between N31 of CDR-L1 and K166 and I168 of CD30L in the cryoEM structure. FIG. 6I shows the close-up view of the contact site between R54 of CDR-H2 and D234 of CD30L in the cryoEM structure. FIG. 6J shows the close-up view of the contact site between G28 of CDR-L1 and S217 of CD30L in the cryoEM structure. FIG. 6K shows the close-up view of the contact site between Y91 of CDR-L3 and H167 of CD30L in the cryoEM structure.

FIG. 7A depicts percentage of body weight changes in the murine colitis model under various treatment conditions as indicated. CD30L blockade by Clone_111 (prophylacticly or therapeutically) were effective in preventive colitis induced weight loss. FIG. 7B depicts endoscopy scores in the murine colitis model under various treatment conditions as indicated. Statistical significance was tested by one-way ANOVA followed by Dunnett's multiple comparisons test using the PBS-treated group as the comparison. * indicates p<0.05 and ** indicates p<0.01. Clone_111 performed better than the positive control of anti-IL-12p40 treatment and were effective at reducing macroscopic colon pathology, with the majority of animals exhibiting normal colons at a macroscopic level. FIG. 7C depicts colon weight:length ratios in the murine colitis model under various treatment conditions as indicated. Statistical significance was tested by one-way ANOVA followed by Dunnett's multiple comparisons test using the PBS-treated group as the comparison. * indicates p<0.05, ** indicates p<0.01, and *** indicates p<0.001. Anti-IL-12p40 treatment positive control significantly reduced colon weight:length ratios, as did prophylactic and therapeutic CD30L blockade with Clone_111. FIG. 7D depicts histopathology sum score in the murine colitis model under various treatment conditions as indicated. Statistical significance was similarly tested. * indicates p<0.05, ** indicates p<0.01, and *** indicates p<0.001. CD30L blockade by Clone_111 prevented microscopic intestinal pathology to a similar extent as the positive control of anti-IL-12p40, and this effect was observed not only in prophylactic CD30L blockade (anti-CD30L treatment before or at the onset of T cell transfer), but also for therapeutic CD30L blockade (anti-CD30L treatment after the induction of colitis), indicating an ongoing role for CD30-CD30L interactions during the development of colitis. FIGS. 7E-7I show representative images from the proximal colon of naïve, isotype control, and Clone_111 treated mice, as indicated in the figures (left Naïve (7E), middle 2 panels isotype control treated (7F and 7G), and right 2 panels Clone_111 treated (7H and 7I)). The images in FIGS. 7E-7I confirmed that CD30L blockade reduced and/or reversed intestinal pathology in colitis. In FIGS. 7F and 7G, H indicates hyperplasia, E indicates Edema, and M indicates affected mucosa. FIGS. 7J-7S depict cytokine levels (IFNg in 7J, IL10 in 7K, IL1b in 7L, IL2 in 7M, IL4 in 7N, IL5 in 7O, IL6 in 7P, KC/GRO in 7Q, TNFα in 7R, IL12p70 in 7S) in the murine colitis model under various treatment conditions as indicated. Statistical significance was similarly tested. * indicates p<0.05, ** indicates p<0.01, and *** indicates p<0.001. The positive control anti-IL-12p40 treatment significantly reduced all but IL-12p70, with a concomitant increase in the type 2 cytokines IL-4 and IL-5. Prophylactic or therapeutic CD30L blockade with Clone_111 significantly reduced IFNg, IL-2, IL-6, KC/Gro, and TNFa, without effecting levels of IL-4 or IL-5. These data suggest that the observed anti-colitogenic effect of CD30L blockade may be due to inhibition of pathogenic immune responses in a mechanism distinct from that of IL-12p40 blockade.

FIG. 8A depicts staining of untransfected cells (negative control), CD30L isoform 1 transfected, and CD30L isoform 2 transfected cells, respectively, stained with AF647 conjugated anti-CD30L antibody (germlined clone 2). Fluorescence minus one (“FMO”) controls are also shown, in which AF647 FMO is FITC Flag-tag stained only; FITC FMO is anti-CD30L-AF647 stained only. In the full panel stain, a clear correlative, FITC+/AF647+ double-positive population is observed for CD30L isoform 1 transfected cells; whereas CD30L isoform 2 transfected cells is only detected by the FITC Flag-tag with virtually no AF647 staining. FIG. 8B shows a schematic representation of membrane-bound CD30L isoform 1 and isoform 2. FIG. 8B also illustrates (1) the Flag-tag and the binding of FITC-conjugated anti-Flag antibody and (2) the binding site for the anti-CD30L antibody (germlined clone 2) on CD30L isoform 1, which is not present on CD30L isoform 2.

### 4. DETAILED DESCRIPTION

There are a limited number of therapies available for autoimmune disorders such as IBD, and the development of new therapeutics presents a challenge in the treatment autoimmune diseases, disorders, and symptoms associated therewith. A notable number anti-inflammatory therapies either do not result in an effective therapeutic response to or fail to provide a lasting response. Furthermore, for the time that a patient is treated with an ineffective anti-inflammatory therapy, the disease worsens. For such non-responding patients, the only treatment is surgery, generally in the form of strictureplasty (reshaping of the intestine) or resection (removal of the intestine). Surgical treatments for IBD are invasive, causing post-operative risks for an estimated one-third of patients undergoing surgery, such as anastomotic leak, infection, and bleeding. Accordingly, the heterogeneity of disease pathogenesis and clinical course, combined with the variable and/or inconsistent response to treatment, suggests a targeted therapeutic approach to treating autoimmune disorders such as IBD is a desirable treatment strategy. Yet very few targeted therapies have been identified and effective in IBD patients, especially those patients who may be non-responsive to existing IBD anti-inflammatory therapies. Thus, there is a need for novel therapeutics to treat autoimmune diseases such as IBD that specifically target IBD pathogenesis.

Described herein are antibodies that target and bind CD30 ligand (CD30L) and, in certain instances, provide an effective targeted therapy approach for the treatment of autoimmune disorders such as IBD. Generally, in an aspect, the antibodies described herein effectively inhibit an interaction between CD30L and CD30 in order to effectively reduce, inhibit, or prevent immune activation (for example, an inflammatory response). The CD30 ligand (also referred to as CD30L, CD153 or TNFSF8) is a member of the tumor necrosis factor (TNF) family. CD30L is a transmembrane protein expressed on immune cells, including activated T and B cells. Generally, after interaction with its receptor CD30, CD30L may induce signal transduction through modulation of TNF receptor-associated factor 1 (TRAF1), TRAF2, 3 and 5. Interaction between CD30-CD30L results in the triggering of immune cell (for example, T cell) activation (for example, stimulating immune cell proliferation, release of cytokines, etc.) and the activation of an inflammatory response. Accordingly, the antibodies described here that target CD30L are useful in preventing, inhibiting, or reducing CD30-CD30L-mediated immune cell activation and/or activation of an inflammatory response in an individual. In certain instances, preventing, inhibiting, or reducing CD30-CD30L-mediated immune cell activation and/or activation of an inflammatory response in an individual is useful for the treatment of autoimmune disorders such as IBD.

### 4.1 anti-CD30L Antibodies

Described herein are recombinant antibodies or antigen binding fragments thereof that bind CD30 ligand (CD30L). CD30 ligand or CD30L or human CD30L (also known and referred to as Tumor necrosis factor ligand superfamily member 8, CD153; CD30L; CD30LG; TNLG3A) as used herein refers to any protein that comprises the expressed and processed forms of the human (Homo sapiens) CD30L gene which, in certain embodiments, is designated as UniProtKB/Swiss-Prot P32971 (NCBI Reference Sequence: NM 001244.4→NP_001235.1 or NM_001252290.1→NP_001239219.1) The term “CD30L” as used herein includes the wild type protein and all naturally occurring variants and/or isoforms thereof, and all transcriptional variants, post-translationally-modified variants (for example, as described as part of the record in UniProtKB/Swiss-Prot P32971). CD30L can be an isoform of CD30L. Such isoforms CD30L are results of an alternate splice site in the coding region of the last exon. In some embodiments of the anti-CD30L antibody or antigen binding fragment thereof provided herein including in Section 4.1, the CD30L that is the specific target of the antibody or antigen binding fragment is the isoform 1 of CD30L. In some embodiments of the anti-CD30L antibody or antigen binding fragment thereof provided herein including in Section 4.1, the CD30L that is the specific target of the antibody or antigen binding fragment is the isoform 1 of CD30L, wherein isoform 1 comprises amino acid sequence set forth in SEQ ID NO:34. In certain embodiments of the anti-CD30L antibody or antigen binding fragment thereof provided herein including in Section 4.1, the CD30L that is the specific target of the antibody or antigen binding fragment is the isoform 2 of CD30L. In certain embodiments of the anti-CD30L antibody or antigen binding fragment thereof provided herein including in Section 4.1, the CD30L that is the specific target of the antibody or antigen binding fragment is the isoform 2 of CD30L, wherein isoform 2 comprises amino acid sequence set forth in SEQ ID NO:35. Isoform 1 of CD30L is described in further details in NM_001244.4 (mRNA and protein sequence) and NP_001235.1 (protein sequence), which are incorporated herein in their entireties by reference. Isoform 2 of CD30L is described in further details in NM_001252290.1 (mRNA and protein sequence) and NP_001239219.1 (protein sequence), which are incorporated herein in their entireties by reference. The antibodies further block an interaction between CD30L and CD30 and inhibit pro-inflammatory cytokine release.

The term “and/or” as used in a phrase with a list of members is intended to include all members individually and all combination of full or partial list of members. For example, a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The disclosure provides that wherever embodiments are provided herein with the term “comprising,” the analogous embodiments described in terms of “consisting of” and/or “consisting essentially of” are also provided, if such analogous embodiments are not explicitly provided. The disclosure further provides that wherever embodiments are described herein with the phrase “consisting essentially of,” the analogous embodiments described in terms of “consisting of” are also provided. The disclosure also provides that wherever embodiments are described herein with the phrase “consisting of,” the analogous embodiments described in terms of “consisting essentially of” are also provided.

All applications, publications, patents and other references, GenBank citations and ATCC citations cited herein are incorporated by reference in their entirety. In case of conflict, the specification, including definitions, will control.

As used herein, numerical values are often presented in a range format throughout this document. The use of a range format is merely for convenience and brevity and should not be construed as an inflexible limitation on the scope of the invention unless the context clearly indicates otherwise. Accordingly, the use of a range expressly includes all possible subranges, all individual numerical values within that range, and all numerical values or numerical ranges including integers within such ranges and fractions of the values or the integers within ranges unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

In addition, reference to a range of 1-3, 3-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-225, 225-250 includes 1, 2, 3, 4, 5, 6, 7, 8, 9, 10, 11, 12, 13, 14, 15, 16, 17, 18, 19, 20, etc. In a further example, reference to a range of 25-250, 250-500, 500-1,000, 1,000-2,500, 2,500-5,000, 5,000-25,000, 25,000-50,000 includes any numerical value or range within or encompassing such values, e.g., 25, 26, 27, 28, 29 . . . 250, 251, 252, 253, 254 . . . 500, 501, 502, 503, 504 . . . , etc.

As also used herein a series of ranges are disclosed throughout this document. The use of a series of ranges includes combinations of the upper and lower ranges to provide another range. This construction applies regardless of the breadth of the range and in all contexts throughout this patent document. Thus, for example, reference to a series of ranges such as 5-10, 10-20, 20-30, 30-40, 40-50, 50-75, 75-100, 100-150, includes ranges such as 5-20, 5-30, 5-40, 5-50, 5-75, 5-100, 5-150, and 10-30, 10-40, 10-50, 10-75, 10-100, 10-150, and 20-40, 20-50, 20-75, 20-100, 20-150, and so forth.

In some embodiments, the term “about” means mean within 10%, within 9%, within 8%, within 7%, within 6%, within 5%, within 4%, within 3%, within 2%, within 1%, or less of a given value, amount, or range. For instance, an antibody variable region comprising about 90% identity to a reference variable region may comprise 82% to 98% identity to the reference variable region.

The terms “specifically bind to,” “specific binding,” and analogous terms when used in the context of one molecule binding to the other, means that one molecule binds to the other molecule with significantly higher affinity than to any cross-reactive antigen or off-target antigen (together as non-target antigen) as determined using experimental techniques, such as Surface Plasmon Resonance (SPR), fluorescence activated cell sorting (FACS) analysis, Kinetic Exclusion Assay (KinExA), isothermal titration calorimetry (ITC), radioimmunoassays (RIA) and enzyme linked immunosorbent assays (ELISAs). Typically, a specific or selective reaction will be at least twice non-target signal or noise of non-target binding and may be more than 10 times non-target binding. See, e.g., Fundamental Immunology 332-36 (Paul ed., 2d ed. 1989) for a discussion regarding antibody specificity. An antibody or antigen binding fragment which binds a target of interest (e.g., a target CD30L) is one that binds the target with sufficient affinity such that the antibody or antigen binding fragment is useful as a therapeutic agent in targeting a cell or tissue expressing the target, and does not significantly cross-react with other proteins. In such embodiments, the extent of binding of the antibody or antigen binding fragment to a “non-target” protein will be less than about 10% of the binding of the antibody or antigen binding fragment to its particular target protein, for example, as determined by FACS analysis, SPR, KinExA, ITC, ELISA, or RIA. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. As such, the term “specific binding,” “specifically binds to,” “specifically inhibits,” “specifically blocks” or “is specific for” a particular target as used herein refers to binding, blocking, or inhibition where a molecule binds to, blocks, or inhibits a particular target without substantially binding to or inhibiting a non-target. In certain embodiments, the antiCD30L antibody or antigen binding fragment provided herein specifically binds to CD30L. In some embodiments, an antiCD30L antibody or antigen binding fragment that specifically binds to CD30L indicates that the antibody reacts or associates more frequently, more rapidly, with greater duration, with greater affinity, or with some combination of the above to the CD30L than with alternative substances, including unrelated proteins.

As used herein, the term “inhibit,” “inhibiting,” or “inhibition” when used in reference to CD30-CD30L binding or interaction, CD30L-mediated CD30 signaling, or other CD30L biochemical or biological functions, is intended to mean decreasing, attenuating, lowering, reducing, or completely abolishing the CD30-CD30L binding or interaction, CD30L-mediated CD30 signaling, or other CD30L biochemical or biological functions (such as CD30L-mediated IL-8 release in CD30 expressing cells). For example, such inhibition of the CD30-CD30L binding or interaction, CD30L-mediated CD30 signaling, or other CD30L biochemical or biological functions can be a reduction of 10%, 20%, 30%, 40%, 50%, 60%, 70%, 80%, 90%, 95%, or 99% of the CD30-CD30L binding or interaction, CD30L-mediated CD30 signaling, or other CD30L biochemical or biological functions. In other examples, such inhibition of the CD30-CD30L binding or interaction, CD30L-mediated CD30 signaling, or other CD30L biochemical or biological functions can be complete elimination of the CD30-CD30L binding or interaction, CD30L-mediated CD30 signaling, or other CD30L biochemical or biological functions. In one embodiment, the anti-CD30L antibody or antigen binding fragment provided herein inhibited the CD30-CD30L binding or interaction, CD30L-mediated CD30 signaling, or other CD30L biochemical or biological functions by at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%. In another embodiment, the anti-CD30L antibody or antigen binding fragment provided herein inhibited the CD30-CD30L binding or interaction, CD30L-mediated CD30 signaling, or other CD30L biochemical or biological functions by about 30%, about 35%, about 40%, about 45%, about 50%, about 55%, about 60%, about 65%, about 70%, about 75%, about 80%, about 85%, about 90%, about 95%, or about 99%. In yet another embodiment, the anti-CD30L antibody or antigen binding fragment provided herein completely abolished the CD30-CD30L binding or interaction, CD30L-mediated CD30 signaling, or other CD30L biochemical or biological functions.

As used herein, the term “block,” or “blocking,” when used in reference to CD30-CD30L binding or interaction, CD30L-mediated CD30 signaling, or other CD30L biochemical or biological functions, is intended to mean decreasing, attenuating, lowering, reducing, or completely abolishing the CD30-CD30L binding or interaction, CD30L-mediated CD30 signaling, or other CD30L biochemical or biological functions (such as CD30L-mediated IL-8 release in CD30 expressing cells), to such a degree that the remaining the CD30-CD30L binding or interaction, CD30L-mediated CD30 signaling, or other CD30L biochemical or biological functions are no longer biologically significant. For example, such blocking of the CD30-CD30L binding or interaction, CD30L-mediated CD30 signaling, or other CD30L biochemical or biological functions can be a reduction of 60%, 65%, 70%, 75%, 80%, 85%, 90%, 95%, or 99% of the CD30-CD30L binding or interaction, CD30L-mediated CD30 signaling, or other CD30L biochemical or biological functions. In other examples, such blocking of the CD30-CD30L binding or interaction, CD30L-mediated CD30 signaling, or other CD30L biochemical or biological functions can be complete elimination of the CD30-CD30L binding or interaction, CD30L-mediated CD30 signaling, or other CD30L biochemical or biological functions. In one embodiment, the anti-CD30L antibody or antigen binding fragment provided herein blocks the CD30-CD30L binding or interaction, CD30L-mediated CD30 signaling, or other CD30L biochemical or biological functions by decreasing at least 60%, at least 65%, at least 70%, at least 75%, at least 80%, at least 85%, at least 90%, at least 95%, or at least 99% of the CD30-CD30L binding or interaction, CD30L-mediated CD30 signaling, or other CD30L biochemical or biological functions, such that the remaining CD30-CD30L binding or interaction, CD30L-mediated CD30 signaling, or other CD30L biochemical or biological functions are no longer biologically significant. In another embodiment, the anti-CD30L antibody or antigen binding fragment provided herein blocks the CD30-CD30L binding or interaction, CD30L-mediated CD30 signaling, or other CD30L biochemical or biological functions by about 60%, about 65%, about 70%, about 75%, about 80%, about 85%, about 90%, about 95%, or about 99% of the CD30-CD30L binding or interaction, CD30L-mediated CD30 signaling, or other CD30L biochemical or biological functions, such that the remaining CD30-CD30L binding or interaction, CD30L-mediated CD30 signaling, or other CD30L biochemical or biological functions are no longer biologically significant. In yet another embodiment, the anti-CD30L antibody or antigen binding fragment provided herein completely blocks, e.g. completely abolishes the CD30-CD30L binding or interaction, CD30L-mediated CD30 signaling, or other CD30L biochemical or biological functions.

“Antibody-dependent cell-mediated cytotoxicity” or “ADCC” refers to a form of cytotoxicity in which secreted immunoglobulin bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. NK cells, the primary cells for mediating ADCC, express FcγRIII only, whereas monocytes express FcγRI, FcγRII, and FcγRIII. FcR expression on hematopoietic cells is known (see, e.g., Ravetch and Kinet, 1991, Annu. Rev. Immunol. 9:457-92). To assess ADCC activity of a molecule of interest, an in vitro ADCC assay (see, e.g., U.S. Pat. Nos. 5,500,362 and 5,821,337) can be performed. Useful effector cells for such assays include peripheral blood mononuclear cells (PBMC) and Natural Killer (NK) cells. Alternatively, or additionally, ADCC activity of the molecule of interest may be assessed in vivo, for example, in an animal model (see, e.g., Clynes et al., 1998, Proc. Natl. Acad. Sci. USA 95:652-56). Antibodies with little or no ADCC activity can be selected for use.

“Antibody-dependent cellular phagocytosis” or “ADCP” refers to the destruction of target cells via monocyte or macrophage-mediated phagocytosis when immunoglobulin bound onto Fc receptors (FcRs) present on certain phagocytotic cells (e.g., neutrophils, monocytes, and macrophages) enable these phagocytotic cells to bind specifically to an antigen-bearing target cell and subsequently kill the target cell. To assess ADCP activity of a molecule of interest, an in vitro ADCP assay (see, e.g., Bracher et al., 2007, J. Immunol. Methods 323:160-71) can be performed. Useful phagocytotic cells for such assays include peripheral blood mononuclear cells (PBMC), purified monocytes from PBMC, or U937 cells differentiated to the mononuclear type. Alternatively, or additionally, ADCP activity of the molecule of interest may be assessed in vivo, for example, in an animal model (see, e.g., Wallace et al., 2001, J. Immunol. Methods 248:167-82). Antibodies with little or no ADCP activity can be selected for use.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay (see, e.g., Gazzano-Santoro et al., 1996, J. Immunol. Methods 202:163) may be performed. Polypeptide variants with altered Fc region amino acid sequences (polypeptides with a variant Fc region) and increased or decreased C1q binding capability have been described (see, e.g., U.S. Pat. No. 6,194,551; WO 1999/51642; Idusogie et al., 2000, J. Immunol. 164:4178-84). Antibodies with little or no CDC activity can be selected for use.

As provided herein, the term “antibody” is used in the broadest sense and includes polyclonal and monoclonal antibodies, including intact antibodies and functional (antigen-binding) antibody fragments thereof, including fragment antigen binding (Fab) fragments, F(ab′)₂ fragments, Fab′ fragments, Fv fragments, recombinant IgG (rIgG) fragments, single chain antibody fragments, including single chain variable fragments (sFv or scFv), and single domain antibodies (for example, sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, for example, bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region.

The provided antibodies are useful as monoclonal antibodies, in polyclonal antibody compositions, as multispecific antibodies (for example, bispecific antibodies and polyreactive antibodies), and as antibody fragments (for example, scFv formats). The antibodies provided herein also include antibody-conjugates and molecules comprising the antibodies, such as chimeric molecules. Thus, an antibody includes, but is not limited to, full-length and native antibodies, as well as fragments and portions thereof retaining the binding specificities thereof, such as any specific binding portion thereof including those having any number of, immunoglobulin classes and/or isotypes (for example, IgG1, IgG2, IgG3, IgG4, IgA, IgD, IgE and IgM); and biologically relevant (antigen-binding) fragments or specific binding portions thereof, including but not limited to Fab, F(ab′)₂, Fv, and scFv (single chain or related entity). A monoclonal antibody is generally one within a composition of substantially homogeneous antibodies; thus, any individual antibodies comprised within the monoclonal antibody composition are identical except for possible naturally occurring mutations that may be present in minor amounts. A polyclonal antibody is a preparation that includes different antibodies of varying sequences that generally are directed against two or more different determinants (epitopes). The monoclonal antibody can comprise a human IgG1 constant region. The monoclonal antibody can comprise a human IgG4 constant region. The term “anti-CD30L” is used as an abbreviation for anti-CD30L antibody or antigen binding fragment thereof.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745. (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme). The CDRs of the antibodies described herein may be defined by the Kabat, IMGT, Chothia, AbM, Aho, contact numbering scheme, or any combination thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The various numbering schemes and the CDR boundaries according to each numbering schemes are summarized below in Table 26.

TABLE 26
CDR boundaries according to various numbering schemes

| | IMGT | Kabat | AbM | Chothia | Contact | Aho |
|---|---|---|---|---|---|---|
| CDR-H1 (VH | 27-38 | 31-35 | 26-35 | 26-32 | 30-35 | 25-40 |
| CDR1) | | | | | | |
| CDR-H2 (VH | 56-65 | 50-65 | 50-58 | 52-56 | 47-58 | 58-77 |
| CDR2) | | | | | | |
| CDR-H3 (VH | 105-117 | 95-102 | 95-102 | 96-101 | 93-101 | 109-137 |
| CDR3) | | | | | | |
| CDR-L1 (VL | 27-38 | 24-34 | 24-34 | 26-32 | 30-36 | 25-40 |
| CDR1) | | | | | | |
| CDR-L2 (VL | 56-65 | 50-56 | 50-56 | 50-52 | 46-55 | 58-77 |
| CDR2) | | | | | | |
| CDR-L3 (VL | 105-117 | 89-97 | 89-97 | 91-96 | 89-96 | 109-137 |
| CDR3) | | | | | | |

Accordingly, the term “variable region residue numbering as in Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHo.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three CDRs (See for example, Kindt et al. Kuby Immunology, 6th ed., W.H. Freeman and Co., page 91(2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively (See for example, Portolano et al., J. Immunol. 150:880-887 (1993); Clarkson et al., Nature 352:624-628 (1991)).

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while μ and ε contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4. A heavy chain can be a human heavy chain.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains. Light chain amino acid sequences are well known in the art. A light chain can be a human light chain.

Among the provided antibodies are antibody fragments. An “antibody fragment,” “antigen-binding fragment,” “antigen-binding domain,” “antigen-binding region,” “antigen binding fragment,” “antigen binding domain,” “antigen binding region,” and similar terms refer to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; diabodies; linear antibodies; single-chain antibody molecules (for example, scFv or sFv); and multispecific antibodies formed from antibody fragments. In particular embodiments, the antibodies are single-chain antibody fragments comprising a variable heavy chain region and/or a variable light chain region, such as scFvs. Generally, an antibody fragment or antigen-binding fragment will comprise one or more CDRs from a parental antibody that are sufficient to confer binding specificity.

As used herein the term “contact” or “contacts” in reference to an antibody binding or being bound to a specific target refers to an amino acid residue of variable region or a CDR coming within 5, 4, 3 or fewer angstroms of the recited contacted residue. Contact includes hydrogen bonding, Van der Waal's interactions and salt bridge formation between an amino acid residue of the variable region or CDR of the antibody and the recited residue.

An “epitope” is the site on the surface of an antigen molecule to which a single antibody molecule binds, such as a localized region on the surface of an antigen, such as a CD30L polypeptide, a CD30L polypeptide fragment, that is capable of being bound to one or more antigen binding regions of an antibody, and that has antigenic or immunogenic activity in an animal, such as a mammal (e.g., a human), that is capable of eliciting an immune response. An epitope having immunogenic activity is a portion of a polypeptide that elicits an antibody response in an animal. An epitope having antigenic activity is a portion of a polypeptide to which an antibody binds as determined by any method well known in the art, including, for example, by an immunoassay. Antigenic epitopes need not necessarily be immunogenic. Epitopes often consist of chemically active surface groupings of molecules such as amino acids or sugar side chains and have specific three dimensional structural characteristics as well as specific charge characteristics. Antibody epitopes can be linear epitopes or conformational epitopes. Linear epitopes are formed by a continuous sequence of amino acids in a protein. Conformational epitopes are formed of amino acids that are discontinuous in the protein sequence, but which are brought together upon folding of the protein into its three-dimensional structure. Induced epitopes are formed when the three dimensional structure of the protein is in an altered conformation, such as following activation or binding of another protein or ligand. In certain embodiments, a CD30L epitope is a three-dimensional surface feature of a CD30L polypeptide. In other embodiments, a CD30L epitope is linear feature of a CD30L polypeptide. Generally an antigen has several or many different epitopes and can react with many different antibodies.

An antibody binds “an epitope,” “essentially the same epitope,” or “the same epitope” as a reference antibody, when the two antibodies recognize identical, overlapping, or adjacent epitopes in a three-dimensional space. The most widely used and rapid methods for determining whether two antibodies bind to identical, overlapping, or adjacent epitopes in a three-dimensional space are competition assays, which can be configured in a number of different formats, for example, using either labeled antigen or labeled antibody. In some assays, the antigen is immobilized on a 96-well plate, or expressed on a cell surface, and the ability of unlabeled antibodies to block the binding of labeled antibodies is measured using radioactive, fluorescent, or enzyme labels.

“Epitope mapping” is the process of identifying the binding sites, or epitopes, of antibodies on their target antigens. “Epitope binning” is the process of grouping antibodies based on the epitopes they recognize. More particularly, epitope binning comprises methods and systems for discriminating the epitope recognition properties of different antibodies, using competition assays combined with computational processes for clustering antibodies based on their epitope recognition properties and identifying antibodies having distinct binding specificities.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, for example, polypeptide linkers, and/or those that are not produced by enzyme digestion of a naturally-occurring intact antibody. In some aspects, the antibody fragments are scFvs.

Generally, a humanized antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (for example, the antibody from which the CDR residues are derived), for example, to restore or improve antibody specificity or affinity. In some embodiments, a humanized antibody refers to forms of non-human (for example, murine) or not fully humanized antibodies having specific immunoglobulin chains, chimeric immunoglobulins, or fragments thereof that contain minimal non-human (for example, murine) sequences. In a non-limiting example, a humanized antibody comprises less than about 40% non-human sequence in the variable region. In some embodiments, a humanized antibody comprises less than about 20% non-human sequence in a full-length antibody sequence. In a further non-limiting example, a humanized antibody comprises less than about 20% non-human sequence in the framework region of each of the heavy chain and light chain variable regions. For instance, the humanized antibody comprises less than about 20%, 19%, 18%, 17%, 16%, 15%, 14%, 13%, 12%, 11%, 10%, 9%, 8%, 7%, 6%, 5%, 4%, 3%, 2%, or 1% non-human sequence in the framework region of each of the heavy chain and light chain variable regions. As another example, the humanized antibody comprises about or less than about 15, 14, 13, 12, 11, 10, 9, 8, 7, 6, 5, 4, 3, 2, or 1 non-human sequences in the framework region of each of the heavy chain and light chain variable regions. In some embodiments, humanized antibodies are human immunoglobulins in which residues from the complementarity determining region (CDR) are replaced by residues from the CDR of a non-human species (for example, mouse, rat, rabbit, hamster) that have the desired specificity, affinity, and capability. These humanized antibodies may contain one or more non-human species mutations, for example, the heavy chain comprises about 1, 2, 3, 4, 5, 6, 7, 8, 9, 10, 11, 12, 13, 14, or 15 non-human species mutations in the framework region, and the light chain comprises about 1, 2, 3, 4, 5, 6, 7, 8, 9, 10, 11, 12, 13, 14, or 15 non-human species mutations in the framework region. The humanized heavy chain variable domain may comprise a IGHV3-9, IGHV4-59, or IGHV3-33 framework with no or fewer than about 10, 9, 8, 7, 6, 5, 4, 3, 2, or 1 amino acid mutations. In some embodiments, the heavy chain variable domain comprises a IGHV3-9 framework. n some embodiments, the heavy chain variable domain comprises a IGHV4-59 framework. n some embodiments, the heavy chain variable domain comprises a IGHV3-33 framework. The humanized light chain variable domain may comprise IGKV1-16, IGKV1-6, or IGKV2-28 framework with no or fewer than about 10, 9, 8, 7, 6, 5, 4, 3, 2, or 1 amino acid mutations.

Among the provided antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived or selected from human antibody libraries, including phage display and cell-free libraries, containing antibody-encoding sequences derived from a human repertoire. In certain embodiments, a human antibody can have sequence liabilities removed or its affinity increased by successive rounds of selection by a method such as phage display.

The terms “polypeptide” and “protein” are used interchangeably to refer to a polymer of amino acid residues and are not limited to a minimum length. Polypeptides, including the provided antibodies and antibody chains and other peptides, for example, linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In some embodiments, amino acid sequence variants of the antibodies provided herein are contemplated. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the invention and evaluating one or more biological activities of the polypeptide as described herein and/or using any of a number of known techniques. For example, it may be desirable to improve the binding affinity and/or other biological properties of the antibody Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

In some embodiments, antibody variants having one or more amino acid substitutions are provided. Sites of interest for mutagenesis by substitution include the CDRs and FRs. Amino acid substitutions may be introduced into an antibody of interest and the products screened for a desired activity, for example, retained/improved antigen binding, decreased immunogenicity such as reduced ADCC or CDC. In some embodiments, substitutions, insertions, or deletions may occur within one or more CDRs, wherein the substitutions, insertions, or deletions do not substantially reduce antibody binding to antigen. For example, conservative substitutions that do not substantially reduce binding affinity may be made in CDRs. Such alterations may be outside of CDR “hotspots.” In some embodiments of the variant VH and VL sequences, each CDR is unaltered.

Alterations (for example, substitutions) may be made in CDRs, for example, to improve antibody affinity. Such alterations may be made in CDR encoding codons with a high mutation rate during somatic maturation (See for example, Chowdhury, Methods Mol. Biol. 207:179-196 (2008)), and the resulting variant can be tested for binding affinity. Affinity maturation (for example, using error-prone PCR, chain shuffling, randomization of CDRs, or oligonucleotide-directed mutagenesis) can be used to improve antibody affinity (See for example, Hoogenboom et al. in Methods in Molecular Biology 178:1-37 (2001)). CDR residues involved in antigen binding may be specifically identified, for example, using alanine scanning mutagenesis or modeling (See for example, Cunningham and Wells Science, 244:1081-1085 (1989)). CDR-H3 and CDR-L3 in particular are often targeted. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions and deletions include amino- and/or carboxyl-terminal fusions ranging in length from one residue to polypeptides containing a hundred or more residues, as well as intrasequence insertions and deletions of single or multiple amino acid residues. Examples of terminal insertions include an antibody with an N-terminal methionyl residue. Other insertional variants of the antibody molecule include the fusion to the N- or C-terminus of the antibody to an enzyme (for example, for ADEPT) or a polypeptide which increases the serum half-life of the antibody. Examples of intrasequence insertion variants of the antibody molecules include an insertion of 3 amino acids in the light chain. Examples of terminal deletions include an antibody with a deletion of 7 or less amino acids at an end of the light chain.

In some embodiments, the antibodies are altered to increase or decrease their glycosylation (for example, by altering the amino acid sequence such that one or more glycosylation sites are created or removed). A carbohydrate attached to an Fc region of an antibody may be altered. Native antibodies from mammalian cells typically comprise a branched, biantennary oligosaccharide attached by an N-linkage to Asn297 of the CH2 domain of the Fc region (See for example, Wright et al. TIBTECH 15:26-32 (1997)). The oligosaccharide can be various carbohydrates, for example, mannose, N-acetyl glucosamine (GlcNAc), galactose, sialic acid, fucose attached to a GlcNAc in the stem of the biantennar oligosaccharide structure. Modifications of the oligosaccharide in an antibody can be made, for example, to create antibody variants with certain improved properties. Antibody glycosylation variants can alter ADCC and/or CDC function. Cell lines, for example, knockout cell lines and methods of their use can be used to produce defucosylated antibodies, for example, Lec13 CHO cells deficient in protein fucosylation and alpha-1,6-fucosyltransferase gene (FUT8) knockout CHO cells (See for example, Ripka et al. Arch. Biochem. Biophys. 249:533-545 (1986); Yamane-Ohnuki et al. Biotech. Bioeng. 87:614 (2004); Kanda, Y. et al., Biotechnol. Bioeng., 94(4): 680-688 (2006)). Other antibody glycosylation variants are also included (See for example, U.S. Pat. No. 6,602,684).

In some embodiments, one or more amino acid modifications may be introduced into the Fc region of an antibody provided herein, thereby generating an Fc region variant. An Fc region herein is a C-terminal region of an immunoglobulin heavy chain that contains at least a portion of the constant region. An Fc region includes native sequence Fc regions and variant Fc regions. The Fc region variant may comprise a human Fc region sequence (for example, a human IgG1, IgG2, IgG3 or IgG4 Fc region) comprising an amino acid modification (for example, a substitution) at one or more amino acid positions.

In some embodiments, the antibodies of this disclosure are variants that possess some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity). Non-limiting examples of in vitro assays to assess ADCC activity of a molecule of interest is described in U.S. Pat. Nos. 5,500,362 and 5,821,337. Alternatively, non-radioactive assays methods may be employed (for example, ACTI™ and CytoTox 96® non-radioactive cytotoxicity assays). Useful effector cells for such assays include peripheral blood mononuclear cells (PBMC), monocytes, macrophages, and Natural Killer (NK) cells.

Antibodies can have increased half-lives and improved binding to the neonatal Fc receptor (FcRn) (See for example, US 2005/0014934). Such antibodies can comprise an Fc region with one or more substitutions therein which improve binding of the Fc region to FcRn, and include those with substitutions at one or more of Fc region residues: 238, 256, 265, 272, 286, 303, 305, 307, 311, 312, 317, 340, 356, 360, 362, 376, 378, 380, 382, 413, 424 or 434 according to the EU numbering system (See for example, U.S. Pat. No. 7,371,826). Other examples of Fc region variants are also contemplated (See for example, Duncan & Winter, Nature 322:738-40 (1988); U.S. Pat. Nos. 5,648,260 and 5,624,821; and WO94/29351).

Reactive groups can be positioned at sites for conjugation to other moieties, such as drug moieties or linker drug moieties, to create an immunoconjugate. In certain embodiments, the recombinant anti-CD30L antibody provided herein may be further modified to contain additional nonproteinaceous moieties that are known and available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n vinyl pyrrolidone) polyethylene glycol, polypropylene glycol homopolymers, polypropylen oxide/ethylene oxide copolymers, polyoxyethylated polyols (for example, glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if two or more polymers are attached, they can be the same or different molecules.

The antibodies described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, for example, a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (for example, AttP-AttB recombination). Additionally, vectors can comprise sequences derived from transposable elements.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87:2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215:403-410, 1990). Percent homology of sequences can be determined using the most recent version of BLAST, as of the filing date of this application.

The nucleic acids encoding the antibodies described herein can be used to infect, transfect, transform, or otherwise render a suitable cell transgenic for the nucleic acid, thus enabling the production of antibodies for commercial or therapeutic uses. Standard cell lines and methods for the production of antibodies from a large-scale cell culture are known in the art. See for example, Li et al., “Cell culture processes for monoclonal antibody production.” Mabs. 2010 September-October; 2(5): 466-477. In certain embodiments, the cell is a Eukaryotic cell. In certain embodiments, the Eukaryotic cell is a mammalian cell. In certain embodiments, the mammalian cell is a Chines Hamster Ovary cell (CHO) cell, an NS0 murine myeloma cell, a HEK293 (Human Embryonic Kidney 293) cell or a PER. C6® cell. In certain embodiments, the nucleic acid encoding the antibody is integrated into a genomic locus of a cell useful for producing antibodies. In certain embodiments, described herein is a method of making an antibody comprising culturing a cell comprising a nucleic acid encoding an antibody under conditions in vitro sufficient to allow production and secretion of said antibody.

In certain embodiments, described herein, is a master cell bank comprising: (a) a mammalian cell line comprising one or more nucleic acids encoding an antibody described herein integrated at a genomic location; and (b) a cryoprotectant. In certain embodiments, the cryoprotectant comprises glycerol, DMSO, or a combination thereof. In certain embodiments, the master cell bank comprises: (a) a CHO cell line comprising a nucleic acid encoding an antibody with (i) a heavy chain variable region amino acid sequence at least 90% identical to that set forth by SEQ ID NO: 1, 2, 5, 6, 9, 10, 13, 14, 17, 19, 21, 23, 25, 27, 29, or 31; and (ii) a light chain amino acid sequence at least 90% identical to that set forth by SEQ ID NO: 3, 4, 7, 8, 11, 12, 15, 16, 18, 20, 22,24, 26, 28, 30, or 32, integrated at a genomic location; and (b) a cryoprotectant. In certain embodiments, the cryoprotectant comprises glycerol, DMSO, or a combination thereof. In certain embodiments, the master cell bank is contained in a suitable vial or container able to withstand freezing by liquid nitrogen.

Also described herein are methods of making an antibody described herein. Such methods comprise incubating a cell or cell-line comprising a nucleic acid encoding the antibody in a cell culture medium under conditions sufficient to allow for expression and secretion of the antibody, and further harvesting the antibody from the cell culture medium. The harvesting can further comprise one or more purification steps to remove live cells, cellular debris, non-antibody proteins or polypeptides, undesired salts, buffers, and medium components. In certain embodiments, the additional purification step(s) include centrifugation, ultracentrifugation, dialysis, desalting, protein A, protein G, protein A/G, or protein L purification, and/or ion exchange chromatography.

The recombinant antibodies or antibody fragments thereof disclosed herein, specifically bind CD30L and are marked by a high affinity for CD30L. Accordingly, the antibodies disclosed herein are useful for targeting (i.e. binding) CD30L. In some embodiments, the anti-CD30L antibodies provided comprise a heavy chain comprising four heavy chain framework regions (HCFR) and three heavy chain complementarity-determining regions (HCDR): HCFR1, HCDR1, HCFR2, HCDR2, HCFR3, HCDR3, and HCFR4; and a light chain comprising four light chain framework regions (LCFR) and three light chain complementarity-determining regions (LCDR): LCFR1, LCDR1, LCFR2, LCDR2, LCFR3, LCDR3, and LCFR4.

In one aspect, provided herein is an antibody or antigen binding fragment thereof that binds CD30L, wherein the antibody or antigen binding fragment thereof comprises: (a) an immunoglobulin heavy chain CDR1 (CDR-H1) comprising the amino acid sequence set forth in any one of SEQ ID NOs: 100-139, 220-234, 465-489, 628-641, and 712-723; (b) an immunoglobulin heavy chain CDR2 (CDR-H2) comprising the amino acid sequence set forth in any one of SEQ ID NOs: 140-179, 235-249, 490-499, 513-527, 642-655, and 724-735; (c) an immunoglobulin heavy chain CDR3 (CDR-H3) comprising the amino acid sequence set forth in any one of SEQ Id NOs: 180-219, 250-264, 528-552, 656-669, and 736-743; (d) an immunoglobulin light chain CDR1 (CDR-L1) comprising the amino acid sequence set forth in any one of SEQ ID NOs: 300-339, 420-434, 553-577, 670-683, and 744-751; (d) an immunoglobulin light chain CDR2 (CDR-L2) comprising the amino acid sequence set forth in any one of SEQ ID NOs: 340-379, 435-449, 578-602, 684-697, and 752-759; and/or (e) an immunoglobulin light chain CDR3 (CDR-L3) comprising the amino acid sequence set forth in any one of SEQ ID NOs: 380-419, 450-464, 603-627, 698-711, and 760-765.

Consistent with the above aspect, in one embodiment, the Antibody or antigen binding fragment thereof comprises or consists of a CDR-H1 comprising the amino acid sequence set forth in any one of SEQ ID NOs: 100-139, 220-234, 465-489, 628-641, and 712-723. In some embodiments, the Antibody or antigen binding fragment thereof comprises or consists of a CDR-H2 comprising the amino acid sequence set forth in any one of SEQ ID NOs: 140-179, 235-249, 490-499, 513-527, 642-655, and 724-735. In certain embodiments, the Antibody or antigen binding fragment thereof comprises or consists of a CDR-H3 comprising the amino acid sequence set forth in any one of SEQ Id NOs: 180-219, 250-264, 528-552, 656-669, and 736-743. In other embodiments, the Antibody or antigen binding fragment thereof comprises or consists of a CDR-L1 comprising the amino acid sequence set forth in any one of SEQ ID NOs: 300-339, 420-434, 553-577, 670-683, and 744-751. In yet other embodiments, the antibody or antigen binding fragment thereof comprises or consists of a CDR-L2 comprising the amino acid sequence set forth in any one of SEQ ID NOs: 340-379, 435-449, 578-602, 684-697, and 752-759. In another embodiment, the antibody or antigen binding fragment thereof comprises or consists of a CDR-L3 comprising the amino acid sequence set forth in any one of SEQ ID NOs: 380-419, 450-464, 603-627, 698-711, and 760-765. In one embodiment, the antibody or antigen binding fragment thereof comprises or consists of a CDR-H1 comprising the amino acid sequence set forth in any one of SEQ ID NOs: 100-139, 220-234, 465-489, 628-641, and 712-723 and a CDR-H2 comprising the amino acid sequence set forth in any one of SEQ ID NOs: 140-179, 235-249, 490-499, 513-527, 642-655, and 724-735. In some embodiments, the antibody or antigen binding fragment thereof comprises or consists of a CDR-H1 comprising the amino acid sequence set forth in any one of SEQ ID NOs: 100-139, 220-234, 465-489, 628-641, and 712-723 and a CDR-H3 comprising the amino acid sequence set forth in any one of SEQ Id NOs: 180-219, 250-264, 528-552, 656-669, and 736-743. In certain embodiments, the antibody or antigen binding fragment thereof comprises or consists of a CDR-H1 comprising the amino acid sequence set forth in any one of SEQ ID NOs: 100-139, 220-234, 465-489, 628-641, and 712-723 and a CDR-L1 comprising the amino acid sequence set forth in any one of SEQ ID NOs: 300-339, 420-434, 553-577, 670-683, and 744-751. In other embodiments, the antibody or antigen binding fragment thereof comprises or consists of a CDR-H1 comprising the amino acid sequence set forth in any one of SEQ ID NOs: 100-139, 220-234, 465-489, 628-641, and 712-723 and a CDR-L2 comprising the amino acid sequence set forth in any one of SEQ ID NOs: 340-379, 435-449, 578-602, 684-697, and 752-759. In some embodiments, the antibody or antigen binding fragment thereof comprises or consists of a CDR-H1 comprising the amino acid sequence set forth in any one of SEQ ID NOs: 100-139, 220-234, 465-489, 628-641, and 712-723 and a CDR-L3 comprising the amino acid sequence set forth in any one of SEQ ID NOs: 380-419, 450-464, 603-627, 698-711, and 760-765. In further embodiments, the antibody or antigen binding fragment thereof comprises or consists of a CDR-H2 comprising the amino acid sequence set forth in any one of SEQ ID NOs: 140-179, 235-249, 490-499, 513-527, 642-655, and 724-735 and a CDR-H3 comprising the amino acid sequence set forth in any one of SEQ Id NOs: 180-219, 250-264, 528-552, 656-669, and 736-743. In one embodiment, the antibody or antigen binding fragment thereof comprises or consists of a CDR-H2 comprising the amino acid sequence set forth in any one of SEQ ID NOs: 140-179, 235-249, 490-499, 513-527, 642-655, and 724-735 and a CDR-L1 comprising the amino acid sequence set forth in any one of SEQ ID NOs: 300-339, 420-434, 553-577, 670-683, and 744-751. In some embodiments, the antibody or antigen binding fragment thereof comprises or consists of a CDR-H2 comprising the amino acid sequence set forth in any one of SEQ ID NOs: 140-179, 235-249, 490-499, 513-527, 642-655, and 724-735 and a CDR-L2 comprising the amino acid sequence set forth in any one of SEQ ID NOs: 340-379, 435-449, 578-602, 684-697, and 752-759. In certain embodiments, the antibody or antigen binding fragment thereof comprises or consists of a CDR-H2 comprising the amino acid sequence set forth in any one of SEQ ID NOs: 140-179, 235-249, 490-499, 513-527, 642-655, and 724-735 and a CDR-L3 comprising the amino acid sequence set forth in any one of SEQ ID NOs: 380-419, 450-464, 603-627, 698-711, and 760-765. In other embodiments, the antibody or antigen binding fragment thereof comprises or consists of a CDR-H3 comprising the amino acid sequence set forth in any one of SEQ Id NOs: 180-219, 250-264, 528-552, 656-669, and 736-743 and a CDR-L1 comprising the amino acid sequence set forth in any one of SEQ ID NOs: 300-339, 420-434, 553-577, 670-683, and 744-751. In yet other embodiments, the antibody or antigen binding fragment thereof comprises or consists of a CDR-H3 comprising the amino acid sequence set forth in any one of SEQ Id NOs: 180-219, 250-264, 528-552, 656-669, and 736-743 and a CDR-L2 comprising the amino acid sequence set forth in any one of SEQ ID NOs: 340-379, 435-449, 578-602, 684-697, and 752-759. In some embodiments, the antibody or antigen binding fragment thereof comprises or consists of a CDR-H3 comprising the amino acid sequence set forth in any one of SEQ Id NOs: 180-219, 250-264, 528-552, 656-669, and 736-743 and a CDR-L3 comprising the amino acid sequence set forth in any one of SEQ ID NOs: 380-419, 450-464, 603-627, 698-711, and 760-765. In one embodiment, the antibody or antigen binding fragment thereof comprises or consists of a CDR-L1 comprising the amino acid sequence set forth in any one of SEQ ID NOs: 300-339, 420-434, 553-577, 670-683, and 744-751 and a CDR-L2 comprising the amino acid sequence set forth in any one of SEQ ID NOs: 340-379, 435-449, 578-602, 684-697, and 752-759. In another embodiment, the antibody or antigen binding fragment thereof comprises or consists of a CDR-L1 comprising the amino acid sequence set forth in any one of SEQ ID NOs: 300-339, 420-434, 553-577, 670-683, and 744-751 and a CDR-L3 comprising the amino acid sequence set forth in any one of SEQ ID NOs: 380-419, 450-464, 603-627, 698-711, and 760-765. In another embodiment, the antibody or antigen binding fragment thereof comprises or consists of a CDR-L2 comprising the amino acid sequence set forth in any one of SEQ ID NOs: 340-379, 435-449, 578-602, 684-697, and 752-759 and a CDR-L3 comprising the amino acid sequence set forth in any one of SEQ ID NOs: 380-419, 450-464, 603-627, 698-711, and 760-765. In other embodiments, the antibody or antigen binding fragment thereof comprises or consists of a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, and a CDR-H3 comprising the amino acid sequence set forth in any one of SEQ Id NOs: 180-219, 250-264, 528-552, 656-669, and 736-743. In yet other embodiments, the antibody or antigen binding fragment thereof comprises or consists of a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735 and a CDR-L1 comprising the amino acid sequence set forth in any one of SEQ ID NOs: 300-339, 420-434, 553-577, 670-683, and 744-751. In further embodiments, the antibody or antigen binding fragment thereof comprises or consists of a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, and a CDR-L2 comprising the amino acid sequence set forth in any one of SEQ ID NOs: 340-379, 435-449, 578-602, 684-697, and 752-759. In some embodiments, the antibody or antigen binding fragment thereof comprises or consists of a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, and a CDR-L3 comprising the amino acid sequence set forth in any one of SEQ ID NOs: 380-419, 450-464, 603-627, 698-711, and 760-765. In some embodiments, the antibody or antigen binding fragment thereof comprises or consists of a CDR-H1 comprising the amino acid sequence set forth in any one of SEQ ID NOs: 100-139, 220-234, 465-489, 628-641, and 712-723, a CDR-H3 comprising the amino acid sequence set forth in any one of SEQ Id NOs: 180-219, 250-264, 528-552, 656-669, and 736-743 and a CDR-L1 comprising the amino acid sequence set forth in any one of SEQ ID NOs: 300-339, 420-434, 553-577, 670-683, and 744-751. In certain embodiments, the antibody or antigen binding fragment thereof comprises or consists of a CDR-H1 comprising the amino acid sequence set forth in any one of SEQ ID NOs: 100-139, 220-234, 465-489, 628-641, and 712-723, a CDR-H3 comprising the amino acid sequence set forth in any one of SEQ Id NOs: 180-219, 250-264, 528-552, 656-669, and 736-743, and a CDR-L2 comprising the amino acid sequence set forth in any one of SEQ ID NOs: 340-379, 435-449, 578-602, 684-697, and 752-759. In some embodiments, the antibody or antigen binding fragment thereof comprises or consists of a CDR-H1 comprising the amino acid sequence set forth in any one of SEQ ID NOs: 100-139, 220-234, 465-489, 628-641, and 712-723, a CDR-H3 comprising the amino acid sequence set forth in any one of SEQ Id NOs: 180-219, 250-264, 528-552, 656-669, and 736-743, and a CDR-L3 comprising the amino acid sequence set forth in any one of SEQ ID NOs: 380-419, 450-464, 603-627, 698-711, and 760-765. In yet other embodiments, the antibody or antigen binding fragment thereof comprises or consists of a CDR-H1 comprising the amino acid sequence set forth in any one of SEQ ID NOs: 100-139, 220-234, 465-489, 628-641, and 712-723, a CDR-L1 comprising the amino acid sequence set forth in any one of SEQ ID NOs: 300-339, 420-434, 553-577, 670-683, and 744-751 and a CDR-L2 comprising the amino acid sequence set forth in any one of SEQ ID NOs: 340-379, 435-449, 578-602, 684-697, and 752-759. In some embodiments, the antibody or antigen binding fragment thereof comprises or consists of a CDR-H1 comprising the amino acid sequence set forth in any one of SEQ ID NOs: 100-139, 220-234, 465-489, 628-641, and 712-723, a CDR-L1 comprising the amino acid sequence set forth in any one of SEQ ID NOs: 300-339, 420-434, 553-577, 670-683, and 744-751 and a CDR-L3 comprising the amino acid sequence set forth in any one of SEQ ID NOs: 380-419, 450-464, 603-627, 698-711, and 760-765. In some embodiments, the antibody or antigen binding fragment thereof comprises or consists of a CDR-H1 comprising the amino acid sequence set forth in any one of SEQ ID NOs: 100-139, 220-234, 465-489, 628-641, and 712-723, a CDR-L2 comprising the amino acid sequence set forth in any one of SEQ ID NOs: 340-379, 435-449, 578-602, 684-697, and 752-759 and a CDR-L3 comprising the amino acid sequence set forth in any one of SEQ ID NOs: 380-419, 450-464, 603-627, 698-711, and 760-765. In some embodiments, the antibody or antigen binding fragment thereof comprises or consists of a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743 and a CDR-L1 comprising the amino acid sequence set forth in any one of SEQ ID NOs: 300-339, 420-434, 553-577, 670-683, and 744-751. In certain embodiments, the antibody or antigen binding fragment thereof comprises or consists of a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, and a CDR-L2 comprising the amino acid sequence set forth in any one of SEQ ID NOs: 340-379, 435-449, 578-602, 684-697, and 752-759. In some embodiments, the antibody or antigen binding fragment thereof comprises or consists of a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, and a CDR-L3 comprising the amino acid sequence set forth in any one of SEQ ID NOs: 380-419, 450-464, 603-627, 698-711, and 760-765. In other embodiments, the antibody or antigen binding fragment thereof comprises or consists of a CDR-H2 comprising the amino acid sequence set forth in any one of SEQ ID NOs: 140-179, 235-249, 490-499, 513-527, 642-655, and 724-735, a CDR-L1 comprising the amino acid sequence set forth in any one of SEQ ID NOs: 300-339, 420-434, 553-577, 670-683, and 744-751 and a CDR-L2 comprising the amino acid sequence set forth in any one of SEQ ID NOs: 340-379, 435-449, 578-602, 684-697, and 752-759. In yet other embodiments, the antibody or antigen binding fragment thereof comprises or consists of a CDR-H2 comprising the amino acid sequence set forth in any one of SEQ ID NOs: 140-179, 235-249, 490-499, 513-527, 642-655, and 724-735, a CDR-L1 comprising the amino acid sequence set forth in any one of SEQ ID NOs: 300-339, 420-434, 553-577, 670-683, and 744-751 and a CDR-L3 comprising the amino acid sequence set forth in any one of SEQ ID NOs: 380-419, 450-464, 603-627, 698-711, and 760-765. In some embodiments, the antibody or antigen binding fragment thereof comprises or consists of a CDR-H2 comprising the amino acid sequence set forth in any one of SEQ ID NOs: 140-179, 235-249, 490-499, 513-527, 642-655, and 724-735, a CDR-L2 comprising the amino acid sequence set forth in any one of SEQ ID NOs: 340-379, 435-449, 578-602, 684-697, and 752-759 and a CDR-L3 comprising the amino acid sequence set forth in any one of SEQ ID NOs: 380-419, 450-464, 603-627, 698-711, and 760-765. In yet other embodiments, the antibody or antigen binding fragment thereof comprises or consists of a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751 and a CDR-L2 comprising the amino acid sequence set forth in any one of SEQ ID NOs: 340-379, 435-449, 578-602, 684-697, and 752-759. In other embodiments, the antibody or antigen binding fragment thereof comprises or consists of a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751 and a CDR-L3 comprising the amino acid sequence set forth in any one of SEQ ID NOs: 380-419, 450-464, 603-627, 698-711, and 760-765. In certain embodiments, the antibody or antigen binding fragment thereof comprises or consists of a CDR-H3 comprising the amino acid sequence set forth in any one of SEQ Id NOs: 180-219, 250-264, 528-552, 656-669, and 736-743, a CDR-L2 comprising the amino acid sequence set forth in any one of SEQ ID NOs: 340-379, 435-449, 578-602, 684-697, and 752-759 and a CDR-L3 comprising the amino acid sequence set forth in any one of SEQ ID NOs: 380-419, 450-464, 603-627, 698-711, and 760-765. In some embodiments, the antibody or antigen binding fragment thereof comprises or consists of a CDR-L1 comprising the amino acid sequence set forth in any one of SEQ ID NOs: 300-339, 420-434, 553-577, 670-683, and 744-751, a CDR-L2 comprising the amino acid sequence set forth in any one of SEQ ID NOs: 340-379, 435-449, 578-602, 684-697, and 752-759 and a CDR-L3 comprising the amino acid sequence set forth in any one of SEQ ID NOs: 380-419, 450-464, 603-627, 698-711, and 760-765. In other embodiments, the antibody or antigen binding fragment thereof comprises or consists of a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, and a CDR-L1 comprising the amino acid sequence set forth in any one of SEQ ID NOs: 300-339, 420-434, 553-577, 670-683, and 744-751. In further embodiments, the antibody or antigen binding fragment thereof comprises or consists of a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, and a CDR-L2 comprising the amino acid sequence set forth in any one of SEQ ID NOs: 340-379, 435-449, 578-602, 684-697, and 752-759. In some embodiments, the antibody or antigen binding fragment thereof comprises or consists of a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, and a CDR-L3 comprising the amino acid sequence set forth in any one of SEQ ID NOs: 380-419, 450-464, 603-627, 698-711, and 760-765. In some embodiments, the antibody or antigen binding fragment thereof comprises or consists of a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-L1 comprising the amino acid sequence set forth in any one of SEQ ID NOs: 300-339, 420-434, 553-577, 670-683, and 744-751, and a CDR-L2 comprising the amino acid sequence set forth in any one of SEQ ID NOs: 340-379, 435-449, 578-602, 684-697, and 752-759. In some embodiments, the antibody or antigen binding fragment thereof comprises or consists of a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-L1 comprising the amino acid sequence set forth in any one of SEQ ID NOs: 300-339, 420-434, 553-577, 670-683, and 744-751, and a CDR-L3 comprising the amino acid sequence set forth in any one of SEQ ID NOs: 380-419, 450-464, 603-627, 698-711, and 760-765. In some embodiments, the antibody or antigen binding fragment thereof comprises or consists of a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765. In certain embodiments, the antibody or antigen binding fragment thereof comprises or consists of a CDR-H1 comprising the amino acid sequence set forth in any one of SEQ ID NOs: 100-139, 220-234, 465-489, 628-641, and 712-723, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, and a CDR-L2 comprising the amino acid sequence set forth in any one of SEQ ID NOs: 340-379, 435-449, 578-602, 684-697, and 752-759. In certain embodiments, the antibody or antigen binding fragment thereof comprises or consists of a CDR-H1 comprising the amino acid sequence set forth in any one of SEQ ID NOs: 100-139, 220-234, 465-489, 628-641, and 712-723, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, and a CDR-L3 comprising the amino acid sequence set forth in any one of SEQ ID NOs: 380-419, 450-464, 603-627, 698-711, and 760-765. In certain embodiments, the antibody or antigen binding fragment thereof comprises or consists of a CDR-H1 comprising the amino acid sequence set forth in any one of SEQ ID NOs: 100-139, 220-234, 465-489, 628-641, and 712-723, a CDR-H3 comprising the amino acid sequence set forth in any one of SEQ Id NOs: 180-219, 250-264, 528-552, 656-669, and 736-743, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765. In yet other embodiments, the antibody or antigen binding fragment thereof comprises or consists of a CDR-H1 comprising the amino acid sequence set forth in any one of SEQ ID NOs: 100-139, 220-234, 465-489, 628-641, and 712-723, a CDR-L1 comprising the amino acid sequence set forth in any one of SEQ ID NOs: 300-339, 420-434, 553-577, 670-683, and 744-751, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765. In further embodiments, the antibody or antigen binding fragment thereof comprises or consists of a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, and a CDR-L2 comprising the amino acid sequence set forth in any one of SEQ ID NOs: 340-379, 435-449, 578-602, 684-697, and 752-759. In some embodiments, the antibody or antigen binding fragment thereof comprises or consists of a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, and a CDR-L3 comprising the amino acid sequence set forth in any one of SEQ ID NOs: 380-419, 450-464, 603-627, 698-711, and 760-765. In further embodiments, the antibody or antigen binding fragment thereof comprises or consists of a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765. In some embodiments, the antibody or antigen binding fragment thereof comprises or consists of a CDR-H2 comprising the amino acid sequence set forth in any one of SEQ ID NOs: 140-179, 235-249, 490-499, 513-527, 642-655, and 724-735, a CDR-L1 comprising the amino acid sequence set forth in any one of SEQ ID NOs: 300-339, 420-434, 553-577, 670-683, and 744-751, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765. In some embodiments, the antibody or antigen binding fragment thereof comprises or consists of a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765. In certain embodiments, the antibody or antigen binding fragment thereof comprises or consists of a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, and a CDR-L2 comprising the amino acid sequence set forth in any one of SEQ ID NOs: 340-379, 435-449, 578-602, 684-697, and 752-759. In some embodiments, the antibody or antigen binding fragment thereof comprises or consists of a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, and a CDR-L3 comprising the amino acid sequence set forth in any one of SEQ ID NOs: 380-419, 450-464, 603-627, 698-711, and 760-765. In some embodiments, the antibody or antigen binding fragment thereof comprises or consists of a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765. In certain embodiments, the antibody or antigen binding fragment thereof comprises or consists of a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-L1 comprising the amino acid sequence set forth in any one of SEQ ID NOs: 300-339, 420-434, 553-577, 670-683, and 744-751, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765. In certain embodiments, the antibody or antigen binding fragment thereof comprises or consists of a CDR-H1 comprising the amino acid sequence set forth in any one of SEQ ID NOs: 100-139, 220-234, 465-489, 628-641, and 712-723, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765. In some embodiments, the antibody or antigen binding fragment thereof comprises or consists of a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765. In some embodiments, the antibody or antigen binding fragment thereof comprises or consists of a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765. In some embodiments, the antibody or antigen binding fragment thereof comprises or consists of any one of a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765. In some embodiments, the antibody or antigen binding fragment thereof comprises or consists of any two of a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765, in any combination or permutation. In some embodiments, the antibody or antigen binding fragment thereof comprises or consists of any three of a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765, in any combination or permutation. In some embodiments, the antibody or antigen binding fragment thereof comprises or consists of any four of a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765, in any combination or permutation. In some embodiments, the antibody or antigen binding fragment thereof comprises or consists of any five of a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765, in any combination or permutation. In some embodiments, the Antibody or antigen binding fragment thereof comprises or consists of all six of a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765.

In some embodiments of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in any one of SEQ ID NOs: 100-109, 628, 635, and 712-723; (b) a CDR-H2 comprising the amino acid sequence set forth in any one of SEQ ID NOs: 140-149, 642, 649, and 724-735; (c) a CDR-H3 comprising the amino acid sequence set forth in any one of SEQ ID NOs: 180-189, 656, 663, and 736-743; (d) a CDR-L1 comprising the amino acid sequence set forth in any one of SEQ ID NOs: 300-309, 670, 677, and 744-751; (e) a CDR-L2 comprising the amino acid sequence set forth in any one of SEQ ID NOs: 340-349, 684, 691, and 752-759; and/or (f) a CDR-L3 comprising the amino acid sequence set forth in any one of SEQ ID Nos: 380-389, 698, 705, and 760-765.

In certain embodiments of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in any one of SEQ ID NOs: 100-109, 628, 635, and 712-723; (b) a CDR-H2 comprising the amino acid sequence set forth in any one of SEQ ID NOs: 140-149, 642, 649, and 724-735; (c) a CDR-H3 comprising the amino acid sequence set forth in any one of SEQ ID NOs: 180-189, 656, 663, and 736-743; (d) a CDR-L1 comprising the amino acid sequence set forth in any one of SEQ ID NOs: 300-309, 670, 677, and 744-751; (e) a CDR-L2 comprising the amino acid sequence set forth in any one of SEQ ID NOs: 340-349, 684, 691, and 752-759; and/or (f) a CDR-L3 comprising the amino acid sequence set forth in any one of SEQ ID Nos: 380-389, 698, 705, and 760-765.

In certain embodiments of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in any one of SEQ ID NOs: 105-109, 628, 635, and 712-723; (b) a CDR-H2 comprising the amino acid sequence set forth in any one of SEQ ID NOs: 145-149, 642, 649, and 724-735; (c) a CDR-H3 comprising the amino acid sequence set forth in any one of SEQ ID NOs: 185-189, 656, 663, and 736-743; (d) a CDR-L1 comprising the amino acid sequence set forth in any one of SEQ ID NOs: 305-309, 670, 677, and 744-751; (e) a CDR-L2 comprising the amino acid sequence set forth in any one of SEQ ID NOs: 345-349, 684, 691, and 752-759; and/or (f) a CDR-L3 comprising the amino acid sequence set forth in any one of SEQ ID NOs: 385-389, 698, 705, and 760-765.

In one aspect, provided herein is an antibody or antigen binding fragment thereof that binds CD30L, wherein the antibody or antigen binding fragment thereof binds to an epitope comprising one or more amino acids in CD30L selected from the group consisting of N165, K166, I168, K169, and D234, wherein the amino acids are numbered according to the amino acid sequence of CD30L as set forth in SEQ ID NO:33.

In another aspect, provided herein is an antibody or antigen binding fragment thereof that binds CD30L, wherein the antibody or antigen binding fragment thereof binds to an epitope comprising one or more amino acids in CD30L selected from the group consisting of H167, S217, and D118, wherein the amino acids are numbered according to the amino acid sequence of CD30L as set forth in SEQ ID NO:33.

In yet another aspect, provided herein is an antibody or antigen binding fragment thereof that binds CD30L, wherein the antibody or antigen binding fragment thereof binds to an epitope comprising one or more amino acids in CD30L selected from the group consisting of D118, N165, K166, H167, I168, K169, S217, and D234, wherein the amino acids are numbered according to the amino acid sequence of CD30L as set forth in SEQ ID NO: 33.

The anti-CD30L antibody provided herein can binds to epitope of various combinations of amino acids on CD30L as described herein. Accordingly, in one embodiment of the anti-CD30L antibody or antigen binding fragment provided herein, including in Section 2 and this Section (Section 4.1), the antibody or antigen binding fragment thereof binds to an epitope comprising any one amino acid in CD30L selected from the group consisting of N165, K166, I168, K169, and D234. In another embodiment of the anti-CD30L antibody or antigen binding fragment provided herein, including in Section 2 and this Section (Section 4.1), the antibody or antigen binding fragment thereof binds to an epitope comprising any two amino acids in CD30L selected from the group consisting of N165, K166, I168, K169, and D234. In a further embodiment of the anti-CD30L antibody or antigen binding fragment provided herein, including in Section 2 and this Section (Section 4.1), the antibody or antigen binding fragment thereof binds to an epitope comprising any three amino acids in CD30L selected from the group consisting of N165, K166, I168, K169, and D234. In yet another embodiment of the anti-CD30L antibody or antigen binding fragment provided herein, including in Section 2 and this Section (Section 4.1), the antibody or antigen binding fragment thereof binds to an epitope comprising any four amino acids in CD30L selected from the group consisting of N165, K166, I168, K169, and D234. In a further embodiment of the anti-CD30L antibody or antigen binding fragment provided herein, including in Section 2 and this Section (Section 4.1), the antibody or antigen binding fragment thereof binds to an epitope comprising any five amino acids in CD30L selected from the group consisting of N165, K166, I168, K169, and D234. The position of the amino acids in this paragraph are numbered according to the amino acid sequence of CD30L as set forth in SEQ ID NO:33.

Additionally, the anti-CD30L antibody provided herein can bind to various combinations of amino acids on CD30L in addition to the epitope described in the previous paragraph. As such, in one embodiment of the anti-CD30L antibody or antigen binding fragment provided herein, including in Section 2 and this Section (Section 4.1), the antibody or antigen binding fragment thereof binds to an epitope further comprising any one amino acid in CD30L selected from the group consisting of H167, S217, and D118. In another embodiment of the anti-CD30L antibody or antigen binding fragment provided herein, including in Section 2 and this Section (Section 4.1), the antibody or antigen binding fragment thereof binds to an epitope further comprising any two amino acids in CD30L selected from the group consisting of H167, S217, and D118. In yet another embodiment of the anti-CD30L antibody or antigen binding fragment provided herein, including in Section 2 and this Section (Section 4.1), the antibody or antigen binding fragment thereof binds to an epitope further comprising three amino acids in CD30L selected from the group consisting of H167, S217, and D118. Alternatively, the anti-CD30L antibody provided herein can bind to various other combinations of amino acids on CD30L. In one embodiment of the anti-CD30L antibody or antigen binding fragment provided herein, including in Section 2 and this Section (Section 4.1), the antibody or antigen binding fragment thereof binds to an epitope comprising any one amino acid in CD30L selected from the group consisting of H167, S217, and D118. In another embodiment of the anti-CD30L antibody or antigen binding fragment provided herein, including in Section 2 and this Section (Section 4.1), the antibody or antigen binding fragment thereof binds to an epitope comprising any two amino acids in CD30L selected from the group consisting of H167, S217, and D118. In yet another embodiment of the anti-CD30L antibody or antigen binding fragment provided herein, including in Section 2 and this Section (Section 4.1), the antibody or antigen binding fragment thereof binds to an epitope comprising three amino acids in CD30L selected from the group consisting of H167, S217, and D118. The position of the amino acids in this paragraph are numbered according to the amino acid sequence of CD30L as set forth in SEQ ID NO:33.

Similarly, the anti-CD30L antibody provided herein can bind to epitope of various other combinations of amino acids on CD30L as described herein. Accordingly, in one embodiment of the anti-CD30L antibody or antigen binding fragment provided herein, including in Section 2 and this Section (Section 4.1), the antibody or antigen binding fragment thereof binds to an epitope comprising any one amino acid in CD30L selected from the group consisting of D118, N165, K166, H167, I168, K169, S217, and D234. In another embodiment of the anti-CD30L antibody or antigen binding fragment provided herein, including in Section 2 and this Section (Section 4.1), the antibody or antigen binding fragment thereof binds to an epitope comprising any two amino acids in CD30L selected from the group consisting of D118, N165, K166, H167, I168, K169, S217, and D234. In a further embodiment of the anti-CD30L antibody or antigen binding fragment provided herein, including in Section 2 and this Section (Section 4.1), the antibody or antigen binding fragment thereof binds to an epitope comprising any three amino acids in CD30L selected from the group consisting of D118, N165, K166, H167, I168, K169, S217, and D234. In yet another embodiment of the anti-CD30L antibody or antigen binding fragment provided herein, including in Section 2 and this Section (Section 4.1), the antibody or antigen binding fragment thereof binds to an epitope comprising any four amino acids in CD30L selected from the group consisting of D118, N165, K166, H167, I168, K169, S217, and D234. In a further embodiment of the anti-CD30L antibody or antigen binding fragment provided herein, including in Section 2 and this Section (Section 4.1), the antibody or antigen binding fragment thereof binds to an epitope comprising any five amino acids in CD30L selected from the group consisting of D118, N165, K166, H167, I168, K169, S217, and D234. In one embodiment of the anti-CD30L antibody or antigen binding fragment provided herein, including in Section 2 and this Section (Section 4.1), the antibody or antigen binding fragment thereof binds to an epitope comprising any six amino acids in CD30L selected from the group consisting of D118, N165, K166, H167, I168, K169, S217, and D234. In another embodiment of the anti-CD30L antibody or antigen binding fragment provided herein, including in Section 2 and this Section (Section 4.1), the antibody or antigen binding fragment thereof binds to an epitope comprising any seven amino acids in CD30L selected from the group consisting of D118, N165, K166, H167, I168, K169, S217, and D234. In a further embodiment of the anti-CD30L antibody or antigen binding fragment provided herein, including in Section 2 and this Section (Section 4.1), the antibody or antigen binding fragment thereof binds to an epitope comprising eight amino acids in CD30L selected from the group consisting of D118, N165, K166, H167, I168, K169, S217, and D234. The position of the amino acids in this paragraph are numbered according to the amino acid sequence of CD30L as set forth in SEQ ID NO:33.

Furthermore, the anti-CD30L antibody provided herein including in Section 2 and this Section (Section 4.1) can bind to certain specific epitope of specific combinations of amino acids on CD30L as described herein and such epitopes are further provided in this paragraph. Accordingly, in one embodiment, the epitope for the anti-CD30L antibody or antigen binding fragment provided herein comprises or consists of N165. In some embodiments, the epitope for the anti-CD30L antibody or antigen binding fragment provided herein comprises or consists of K166. In certain embodiments, the epitope for the anti-CD30L antibody or antigen binding fragment provided herein comprises or consists of I168. In other embodiments, the epitope for the anti-CD30L antibody or antigen binding fragment provided herein comprises or consists of K169. In yet other embodiments, the epitope for the anti-CD30L antibody or antigen binding fragment provided herein comprises or consists of D234. In another embodiment, the epitope for the anti-CD30L antibody or antigen binding fragment provided herein comprises or consists of H167. In yet another embodiment, the epitope for the anti-CD30L antibody or antigen binding fragment provided herein comprises or consists of S217. In yet another embodiment, the epitope for the anti-CD30L antibody or antigen binding fragment provided herein comprises or consists of D118. In one embodiment, the epitope for the anti-CD30L antibody or antigen binding fragment provided herein comprises or consists of N165 and K166. In some embodiments, the epitope for the anti-CD30L antibody or antigen binding fragment provided herein comprises or consists of N165 and I168. In certain embodiments, the epitope for the anti-CD30L antibody or antigen binding fragment provided herein comprises or consists of N165 and K169. In other embodiments, the epitope for the anti-CD30L antibody or antigen binding fragment provided herein comprises or consists of N165 and D234. In some embodiments, the epitope for the anti-CD30L antibody or antigen binding fragment provided herein comprises or consists of N165 and H167. In other embodiments, the epitope for the anti-CD30L antibody or antigen binding fragment provided herein comprises or consists of N165 and S217. In further embodiments, the epitope for the anti-CD30L antibody or antigen binding fragment provided herein comprises or consists of K166 and I168. In one embodiment, the epitope for the anti-CD30L antibody or antigen binding fragment provided herein comprises or consists of K166 and K169. In some embodiments, the epitope for the anti-CD30L antibody or antigen binding fragment provided herein comprises or consists of K166 and D234. In certain embodiments, the epitope for the anti-CD30L antibody or antigen binding fragment provided herein comprises or consists of K166 and H167. In some embodiments, the epitope for the anti-CD30L antibody or antigen binding fragment provided herein comprises or consists of K166 and S217. In other embodiments, the epitope for the anti-CD30L antibody or antigen binding fragment provided herein comprises or consists of I168 and K169. In yet other embodiments, the epitope for the anti-CD30L antibody or antigen binding fragment provided herein comprises or consists of I168 and D234. In some embodiments, the epitope for the anti-CD30L antibody or antigen binding fragment provided herein comprises or consists of I168 and H167. In certain embodiments, the epitope for the anti-CD30L antibody or antigen binding fragment provided herein comprises or consists of I168 and S217. In one embodiment, the epitope for the anti-CD30L antibody or antigen binding fragment provided herein comprises or consists of K169 and D234. In another embodiment, the epitope for the anti-CD30L antibody or antigen binding fragment provided herein comprises or consists of K169 and H167. In one embodiment, the epitope for the anti-CD30L antibody or antigen binding fragment provided herein comprises or consists of K169 and S217. In some embodiments, the epitope for the anti-CD30L antibody or antigen binding fragment provided herein comprises or consists of D234 and H167. In certain embodiments, the epitope for the anti-CD30L antibody or antigen binding fragment provided herein comprises or consists of D234 and S217. In one embodiment, the epitope for the anti-CD30L antibody or antigen binding fragment provided herein comprises or consists of H167 and S217. In one embodiment, the epitope for the anti-CD30L antibody or antigen binding fragment provided herein comprises or consists of N165 and D118. In another embodiment, the epitope for the anti-CD30L antibody or antigen binding fragment provided herein comprises or consists of K166 and D118. In some embodiments, the epitope for the anti-CD30L antibody or antigen binding fragment provided herein comprises or consists of I168 and D118. In certain embodiments, the epitope for the anti-CD30L antibody or antigen binding fragment provided herein comprises or consists of K169 and D118. In other embodiments, the epitope for the anti-CD30L antibody or antigen binding fragment provided herein comprises or consists of D234 and D118. In some embodiments, the epitope for the anti-CD30L antibody or antigen binding fragment provided herein comprises or consists of H167 and D118. In other embodiments, the epitope for the anti-CD30L antibody or antigen binding fragment provided herein comprises or consists of S217 and D118. In other embodiments, the epitope for the anti-CD30L antibody or antigen binding fragment provided herein comprises or consists of N165, K166, and I168. In yet other embodiments, the epitope for the anti-CD30L antibody or antigen binding fragment provided herein comprises or consists of N165, K166 and K169. In further embodiments, the epitope for the anti-CD30L antibody or antigen binding fragment provided herein comprises or consists of N165, K166, and D234. In some embodiments, the epitope for the anti-CD30L antibody or antigen binding fragment provided herein comprises or consists of N165, K166, and H167. In certain embodiments, the epitope for the anti-CD30L antibody or antigen binding fragment provided herein comprises or consists of N165, K166, and S217. In some embodiments, the epitope for the anti-CD30L antibody or antigen binding fragment provided herein comprises or consists of N165, I168 and K169. In certain embodiments, the epitope for the anti-CD30L antibody or antigen binding fragment provided herein comprises or consists of N165, I168, and D234. In some embodiments, the epitope for the anti-CD30L antibody or antigen binding fragment provided herein comprises or consists of N165, I168, and H167. In other embodiments, the epitope for the anti-CD30L antibody or antigen binding fragment provided herein comprises or consists of N165, I168, and S217. In yet other embodiments, the epitope for the anti-CD30L antibody or antigen binding fragment provided herein comprises or consists of N165, K169 and D234. In some embodiments, the epitope for the anti-CD30L antibody or antigen binding fragment provided herein comprises or consists of N165, K169 and H167. In other embodiments, the epitope for the anti-CD30L antibody or antigen binding fragment provided herein comprises or consists of N165, K169 and S217. In some embodiments, the epitope for the anti-CD30L antibody or antigen binding fragment provided herein comprises or consists of N165, D234 and H167. In certain embodiments, the epitope for the anti-CD30L antibody or antigen binding fragment provided herein comprises or consists of N165, D234 and S217. In other embodiments, the epitope for the anti-CD30L antibody or antigen binding fragment provided herein comprises or consists of N165, H167 and S217. In some embodiments, the epitope for the anti-CD30L antibody or antigen binding fragment provided herein comprises or consists of K166, I168 and K169. In certain embodiments, the epitope for the anti-CD30L antibody or antigen binding fragment provided herein comprises or consists of K166, I168, and D234. In some embodiments, the epitope for the anti-CD30L antibody or antigen binding fragment provided herein comprises or consists of K166, I168, and H167. In certain embodiments, the epitope for the anti-CD30L antibody or antigen binding fragment provided herein comprises or consists of K166, I168, and S217. In other embodiments, the epitope for the anti-CD30L antibody or antigen binding fragment provided herein comprises or consists of K166, K169 and D234. In yet other embodiments, the epitope for the anti-CD30L antibody or antigen binding fragment provided herein comprises or consists of K166, K169 and H167. In some embodiments, the epitope for the anti-CD30L antibody or antigen binding fragment provided herein comprises or consists of K166, K169 and S217. In some embodiments, the epitope for the anti-CD30L antibody or antigen binding fragment provided herein comprises or consists of K166, D234 and H167. In some embodiments, the epitope for the anti-CD30L antibody or antigen binding fragment provided herein comprises or consists of K166, D234 and S217. In some embodiments, the epitope for the anti-CD30L antibody or antigen binding fragment provided herein comprises or consists of K166, H167 and S217. In yet other embodiments, the epitope for the anti-CD30L antibody or antigen binding fragment provided herein comprises or consists of I168, K169 and D234. In other embodiments, the epitope for the anti-CD30L antibody or antigen binding fragment provided herein comprises or consists of I168, K169 and H167. In some embodiments, the epitope for the anti-CD30L antibody or antigen binding fragment provided herein comprises or consists of I168, K169 and S217. In certain embodiments, the epitope for the anti-CD30L antibody or antigen binding fragment provided herein comprises or consists of I168, D234 and H167. In other embodiments, the epitope for the anti-CD30L antibody or antigen binding fragment provided herein comprises or consists of I168, D234 and S217. In yet other embodiments, the epitope for the anti-CD30L antibody or antigen binding fragment provided herein comprises or consists of I168, H167 and S217. In some embodiments, the epitope for the anti-CD30L antibody or antigen binding fragment provided herein comprises or consists of K169, D234 and H167. In some embodiments, the epitope for the anti-CD30L antibody or antigen binding fragment provided herein comprises or consists of K169, D234 and S217. In some embodiments, the epitope for the anti-CD30L antibody or antigen binding fragment provided herein comprises or consists of K169, H167 and S217. In some embodiments, the epitope for the anti-CD30L antibody or antigen binding fragment provided herein comprises or consists of D234, H167 and S217. In one embodiment, the epitope for the anti-CD30L antibody or antigen binding fragment provided herein comprises or consists of N165, K166, and D118. In some embodiments, the epitope for the anti-CD30L antibody or antigen binding fragment provided herein comprises or consists of N165, I168, and D118. In certain embodiments, the epitope for the anti-CD30L antibody or antigen binding fragment provided herein comprises or consists of N165, K169, and D118. In other embodiments, the epitope for the anti-CD30L antibody or antigen binding fragment provided herein comprises or consists of N165, D234, and D118. In some embodiments, the epitope for the anti-CD30L antibody or antigen binding fragment provided herein comprises or consists of N165, H167, and D118. In other embodiments, the epitope for the anti-CD30L antibody or antigen binding fragment provided herein comprises or consists of N165, S217, and D118. In further embodiments, the epitope for the anti-CD30L antibody or antigen binding fragment provided herein comprises or consists of K166, I168, and D118. In one embodiment, the epitope for the anti-CD30L antibody or antigen binding fragment provided herein comprises or consists of K166, K169, and D118. In some embodiments, the epitope for the anti-CD30L antibody or antigen binding fragment provided herein comprises or consists of K166, D234, and D118. In certain embodiments, the epitope for the anti-CD30L antibody or antigen binding fragment provided herein comprises or consists of K166, H167, and D118. In some embodiments, the epitope for the anti-CD30L antibody or antigen binding fragment provided herein comprises or consists of K166, S217, and D118. In other embodiments, the epitope for the anti-CD30L antibody or antigen binding fragment provided herein comprises or consists of I168, K169, and D118. In yet other embodiments, the epitope for the anti-CD30L antibody or antigen binding fragment provided herein comprises or consists of I168, D234, and D118. In some embodiments, the epitope for the anti-CD30L antibody or antigen binding fragment provided herein comprises or consists of I168, H167, and D118. In certain embodiments, the epitope for the anti-CD30L antibody or antigen binding fragment provided herein comprises or consists of I168, S217, and D118. In one embodiment, the epitope for the anti-CD30L antibody or antigen binding fragment provided herein comprises or consists of K169, D234, and D118. In another embodiment, the epitope for the anti-CD30L antibody or antigen binding fragment provided herein comprises or consists of K169, H167, and D118. In one embodiment, the epitope for the anti-CD30L antibody or antigen binding fragment provided herein comprises or consists of K169, S217, and D118. In some embodiments, the epitope for the anti-CD30L antibody or antigen binding fragment provided herein comprises or consists of D234, H167, and D118. In certain embodiments, the epitope for the anti-CD30L antibody or antigen binding fragment provided herein comprises or consists of D234, S217, and D118. In one embodiment, the epitope for the anti-CD30L antibody or antigen binding fragment provided herein comprises or consists of H167, S217, and D118. In other embodiments, the epitope for the anti-CD30L antibody or antigen binding fragment provided herein comprises or consists of N165, K166, I168, and K169. In further embodiments, the epitope for the anti-CD30L antibody or antigen binding fragment provided herein comprises or consists of N165, K166, I168, and D234. In some embodiments, the epitope for the anti-CD30L antibody or antigen binding fragment provided herein comprises or consists of N165, K166, I168, and H167. In other embodiments, the epitope for the anti-CD30L antibody or antigen binding fragment provided herein comprises or consists of N165, K166, I168, and S217. In some embodiments, the epitope for the anti-CD30L antibody or antigen binding fragment provided herein comprises or consists of N165, K166, K169, and D234. In some embodiments, the epitope for the anti-CD30L antibody or antigen binding fragment provided herein comprises or consists of N165, K166, K169, and H167. In some embodiments, the epitope for the anti-CD30L antibody or antigen binding fragment provided herein comprises or consists of N165, K166, K169, and S217. In some embodiments, the epitope for the anti-CD30L antibody or antigen binding fragment provided herein comprises or consists of N165, K166, D234, and H167. In some embodiments, the epitope for the anti-CD30L antibody or antigen binding fragment provided herein comprises or consists of N165, K166, D234, and S217. In some embodiments, the epitope for the anti-CD30L antibody or antigen binding fragment provided herein comprises or consists of N165, K166, H167, and S217. In certain embodiments, the epitope for the anti-CD30L antibody or antigen binding fragment provided herein comprises or consists of N165, I168, K169, and D234. In certain embodiments, the epitope for the anti-CD30L antibody or antigen binding fragment provided herein comprises or consists of N165, I168, K169, and H167. In some embodiments, the epitope for the anti-CD30L antibody or antigen binding fragment provided herein comprises or consists of N165, I168, K169, and S217. In certain embodiments, the epitope for the anti-CD30L antibody or antigen binding fragment provided herein comprises or consists of N165, I168, D234, and H167. In some embodiments, the epitope for the anti-CD30L antibody or antigen binding fragment provided herein comprises or consists of N165, I168, D234, and S217. In other embodiments, the epitope for the anti-CD30L antibody or antigen binding fragment provided herein comprises or consists of N165, I168, H167, and S217. In yet other embodiments, the epitope for the anti-CD30L antibody or antigen binding fragment provided herein comprises or consists of N165, K169, D234, and H167. In some embodiments, the epitope for the anti-CD30L antibody or antigen binding fragment provided herein comprises or consists of N165, K169, D234, and S217. In certain embodiments, the epitope for the anti-CD30L antibody or antigen binding fragment provided herein comprises or consists of N165, K169, H167, and S217. In other embodiments, the epitope for the anti-CD30L antibody or antigen binding fragment provided herein comprises or consists of N165, D234, H167, and S217. In further embodiments, the epitope for the anti-CD30L antibody or antigen binding fragment provided herein comprises or consists of K166, I168, K169, and D234. In some embodiments, the epitope for the anti-CD30L antibody or antigen binding fragment provided herein comprises or consists of K166, I168, K169, and H167. In certain embodiments, the epitope for the anti-CD30L antibody or antigen binding fragment provided herein comprises or consists of K166, I168, K169, and S217. In further embodiments, the epitope for the anti-CD30L antibody or antigen binding fragment provided herein comprises or consists of K166, I168, D234, and H167. In other embodiments, the epitope for the anti-CD30L antibody or antigen binding fragment provided herein comprises or consists of K166, I168, D234, and S217. In some embodiments, the epitope for the anti-CD30L antibody or antigen binding fragment provided herein comprises or consists of K166, I168, H167, and S217. In some embodiments, the epitope for the anti-CD30L antibody or antigen binding fragment provided herein comprises or consists of K166, K169, D234, and H167. In some embodiments, the epitope for the anti-CD30L antibody or antigen binding fragment provided herein comprises or consists of K166, K169, D234, and S217. In some embodiments, the epitope for the anti-CD30L antibody or antigen binding fragment provided herein comprises or consists of K166, K169, H167, and S217. In some embodiments, the epitope for the anti-CD30L antibody or antigen binding fragment provided herein comprises or consists of K166, D234, H167, and S217. In some embodiments, the epitope for the anti-CD30L antibody or antigen binding fragment provided herein comprises or consists of I168, K169, D234, and H167. In some embodiments, the epitope for the anti-CD30L antibody or antigen binding fragment provided herein comprises or consists of I168, K169, D234, and S217. In some embodiments, the epitope for the anti-CD30L antibody or antigen binding fragment provided herein comprises or consists of I168, K169, H167, and S217. In some embodiments, the epitope for the anti-CD30L antibody or antigen binding fragment provided herein comprises or consists of I168, D234, H167, and S217. In some embodiments, the epitope for the anti-CD30L antibody or antigen binding fragment provided herein comprises or consists of K169, D234, H167, and S217. In other embodiments, the epitope for the anti-CD30L antibody or antigen binding fragment provided herein comprises or consists of N165, K166, I168, and D118. In yet other embodiments, the epitope for the anti-CD30L antibody or antigen binding fragment provided herein comprises or consists of N165, K166, K169, and D118. In further embodiments, the epitope for the anti-CD30L antibody or antigen binding fragment provided herein comprises or consists of N165, K166, D234, and D118. In some embodiments, the epitope for the anti-CD30L antibody or antigen binding fragment provided herein comprises or consists of N165, K166, H167, and D118. In certain embodiments, the epitope for the anti-CD30L antibody or antigen binding fragment provided herein comprises or consists of N165, K166, S217, and D118. In some embodiments, the epitope for the anti-CD30L antibody or antigen binding fragment provided herein comprises or consists of N165, I168, K169, and D118. In certain embodiments, the epitope for the anti-CD30L antibody or antigen binding fragment provided herein comprises or consists of N165, I168, D234, and D118. In some embodiments, the epitope for the anti-CD30L antibody or antigen binding fragment provided herein comprises or consists of N165, I168, H167, and D118. In other embodiments, the epitope for the anti-CD30L antibody or antigen binding fragment provided herein comprises or consists of N165, I168, S217, and D118. In yet other embodiments, the epitope for the anti-CD30L antibody or antigen binding fragment provided herein comprises or consists of N165, K169, D234, and D118. In some embodiments, the epitope for the anti-CD30L antibody or antigen binding fragment provided herein comprises or consists of N165, K169, H167, and D118. In other embodiments, the epitope for the anti-CD30L antibody or antigen binding fragment provided herein comprises or consists of N165, K169, S217, and D118. In some embodiments, the epitope for the anti-CD30L antibody or antigen binding fragment provided herein comprises or consists of N165, D234, H167, and D118. In certain embodiments, the epitope for the anti-CD30L antibody or antigen binding fragment provided herein comprises or consists of N165, D234, S217, and D118. In other embodiments, the epitope for the anti-CD30L antibody or antigen binding fragment provided herein comprises or consists of N165, H167, S217, and D118. In some embodiments, the epitope for the anti-CD30L antibody or antigen binding fragment provided herein comprises or consists of K166, I168, K169, and D118. In certain embodiments, the epitope for the anti-CD30L antibody or antigen binding fragment provided herein comprises or consists of K166, I168, D234, and D118. In some embodiments, the epitope for the anti-CD30L antibody or antigen binding fragment provided herein comprises or consists of K166, I168, H167, and D118. In certain embodiments, the epitope for the anti-CD30L antibody or antigen binding fragment provided herein comprises or consists of K166, I168, S217, and D118. In other embodiments, the epitope for the anti-CD30L antibody or antigen binding fragment provided herein comprises or consists of K166, K169, D234, and D118. In yet other embodiments, the epitope for the anti-CD30L antibody or antigen binding fragment provided herein comprises or consists of K166, K169, H167, and D118. In some embodiments, the epitope for the anti-CD30L antibody or antigen binding fragment provided herein comprises or consists of K166, K169, S217, and D118. In some embodiments, the epitope for the anti-CD30L antibody or antigen binding fragment provided herein comprises or consists of K166, D234, H167, and D118. In some embodiments, the epitope for the anti-CD30L antibody or antigen binding fragment provided herein comprises or consists of K166, D234, S217, and D118. In some embodiments, the epitope for the anti-CD30L antibody or antigen binding fragment provided herein comprises or consists of K166, H167, S217, and D118. In yet other embodiments, the epitope for the anti-CD30L antibody or antigen binding fragment provided herein comprises or consists of I168, K169, D234, and D118. In other embodiments, the epitope for the anti-CD30L antibody or antigen binding fragment provided herein comprises or consists of I168, K169, H167, and D118. In some embodiments, the epitope for the anti-CD30L antibody or antigen binding fragment provided herein comprises or consists of I168, K169, S217, and D118. In certain embodiments, the epitope for the anti-CD30L antibody or antigen binding fragment provided herein comprises or consists of I168, D234, H167, and D118. In other embodiments, the epitope for the anti-CD30L antibody or antigen binding fragment provided herein comprises or consists of I168, D234, S217, and D118. In yet other embodiments, the epitope for the anti-CD30L antibody or antigen binding fragment provided herein comprises or consists of I168, H167, S217, and D118. In some embodiments, the epitope for the anti-CD30L antibody or antigen binding fragment provided herein comprises or consists of K169, D234, H167, and D118. In some embodiments, the epitope for the anti-CD30L antibody or antigen binding fragment provided herein comprises or consists of K169, D234, S217, and D118. In some embodiments, the epitope for the anti-CD30L antibody or antigen binding fragment provided herein comprises or consists of K169, H167, S217, and D118. In some embodiments, the epitope for the anti-CD30L antibody or antigen binding fragment provided herein comprises or consists of D234, H167, S217, and D118. In certain embodiments, the epitope for the anti-CD30L antibody or antigen binding fragment provided herein comprises or consists of N165, K166, I168, K169, and D234. In some embodiments, the epitope for the anti-CD30L antibody or antigen binding fragment provided herein comprises or consists of N165, K166, I168, K169, and H167. In certain embodiments, the epitope for the anti-CD30L antibody or antigen binding fragment provided herein comprises or consists of N165, K166, I168, K169, and S217. In some embodiments, the epitope for the anti-CD30L antibody or antigen binding fragment provided herein comprises or consists of N165, K166, I168, D234, and H167. In certain embodiments, the epitope for the anti-CD30L antibody or antigen binding fragment provided herein comprises or consists of N165, K166, I168, D234, and S217. In some embodiments, the epitope for the anti-CD30L antibody or antigen binding fragment provided herein comprises or consists of N165, K166, I168, H167, and S217. In certain embodiments, the epitope for the anti-CD30L antibody or antigen binding fragment provided herein comprises or consists of N165, K166, K169, D234, and H167. In some embodiments, the epitope for the anti-CD30L antibody or antigen binding fragment provided herein comprises or consists of N165, K166, K169, D234, and S217. In certain embodiments, the epitope for the anti-CD30L antibody or antigen binding fragment provided herein comprises or consists of N165, K166, K169, H167, and S217. In some embodiments, the epitope for the anti-CD30L antibody or antigen binding fragment provided herein comprises or consists of N165, K166, D234, H167, and S217. In certain embodiments, the epitope for the anti-CD30L antibody or antigen binding fragment provided herein comprises or consists of N165, I168, K169, D234, and H167. In some embodiments, the epitope for the anti-CD30L antibody or antigen binding fragment provided herein comprises or consists of N165, I168, K169, D234, and S217. In certain embodiments, the epitope for the anti-CD30L antibody or antigen binding fragment provided herein comprises or consists of N165, I168, K169, H167, and S217. In some embodiments, the epitope for the anti-CD30L antibody or antigen binding fragment provided herein comprises or consists of N165, I168, D234, H167, and S217. In certain embodiments, the epitope for the anti-CD30L antibody or antigen binding fragment provided herein comprises or consists of N165, K169, D234, H167, and S217. In some embodiments, the epitope for the anti-CD30L antibody or antigen binding fragment provided herein comprises or consists of K166, I168, K169, D234, and H167. In certain embodiments, the epitope for the anti-CD30L antibody or antigen binding fragment provided herein comprises or consists of K166, I168, K169, D234, and S217. In some embodiments, the epitope for the anti-CD30L antibody or antigen binding fragment provided herein comprises or consists of K166, I168, K169, H167, and S217. In some embodiments, the epitope for the anti-CD30L antibody or antigen binding fragment provided herein comprises or consists of K166, I168, D234, H167, and S217. In certain embodiments, the epitope for the anti-CD30L antibody or antigen binding fragment provided herein comprises or consists of K166, K169, D234, H167, and S217. In some embodiments, the epitope for the anti-CD30L antibody or antigen binding fragment provided herein comprises or consists of I168, K169, D234, H167, and S217. In other embodiments, the epitope for the anti-CD30L antibody or antigen binding fragment provided herein comprises or consists of N165, K166, I168, K169, and D118. In further embodiments, the epitope for the anti-CD30L antibody or antigen binding fragment provided herein comprises or consists of N165, K166, I168, D234, and D118. In some embodiments, the epitope for the anti-CD30L antibody or antigen binding fragment provided herein comprises or consists of N165, K166, I168, H167, and D118. In other embodiments, the epitope for the anti-CD30L antibody or antigen binding fragment provided herein comprises or consists of N165, K166, I168, S217, and D118. In some embodiments, the epitope for the anti-CD30L antibody or antigen binding fragment provided herein comprises or consists of N165, K166, K169, D234, and D118. In some embodiments, the epitope for the anti-CD30L antibody or antigen binding fragment provided herein comprises or consists of N165, K166, K169, H167, and D118. In some embodiments, the epitope for the anti-CD30L antibody or antigen binding fragment provided herein comprises or consists of N165, K166, K169, S217, and D118. In some embodiments, the epitope for the anti-CD30L antibody or antigen binding fragment provided herein comprises or consists of N165, K166, D234, H167, and D118. In some embodiments, the epitope for the anti-CD30L antibody or antigen binding fragment provided herein comprises or consists of N165, K166, D234, S217, and D118. In some embodiments, the epitope for the anti-CD30L antibody or antigen binding fragment provided herein comprises or consists of N165, K166, H167, S217, and D118. In certain embodiments, the epitope for the anti-CD30L antibody or antigen binding fragment provided herein comprises or consists of N165, I168, K169, D234, and D118. In certain embodiments, the epitope for the anti-CD30L antibody or antigen binding fragment provided herein comprises or consists of N165, I168, K169, H167, and D118. In some embodiments, the epitope for the anti-CD30L antibody or antigen binding fragment provided herein comprises or consists of N165, I168, K169, S217, and D118. In certain embodiments, the epitope for the anti-CD30L antibody or antigen binding fragment provided herein comprises or consists of N165, I168, D234, H167, and D118. In some embodiments, the epitope for the anti-CD30L antibody or antigen binding fragment provided herein comprises or consists of N165, I168, D234, S217, and D118. In other embodiments, the epitope for the anti-CD30L antibody or antigen binding fragment provided herein comprises or consists of N165, I168, H167, S217, and D118. In yet other embodiments, the epitope for the anti-CD30L antibody or antigen binding fragment provided herein comprises or consists of N165, K169, D234, H167, and D118. In some embodiments, the epitope for the anti-CD30L antibody or antigen binding fragment provided herein comprises or consists of N165, K169, D234, S217, and D118. In certain embodiments, the epitope for the anti-CD30L antibody or antigen binding fragment provided herein comprises or consists of N165, K169, H167, S217, and D118. In other embodiments, the epitope for the anti-CD30L antibody or antigen binding fragment provided herein comprises or consists of N165, D234, H167, S217, and D118. In further embodiments, the epitope for the anti-CD30L antibody or antigen binding fragment provided herein comprises or consists of K166, I168, K169, D234, and D118. In some embodiments, the epitope for the anti-CD30L antibody or antigen binding fragment provided herein comprises or consists of K166, I168, K169, H167, and D118. In certain embodiments, the epitope for the anti-CD30L antibody or antigen binding fragment provided herein comprises or consists of K166, I168, K169, S217, and D118. In further embodiments, the epitope for the anti-CD30L antibody or antigen binding fragment provided herein comprises or consists of K166, I168, D234, H167, and D118. In other embodiments, the epitope for the anti-CD30L antibody or antigen binding fragment provided herein comprises or consists of K166, I168, D234, S217, and D118. In some embodiments, the epitope for the anti-CD30L antibody or antigen binding fragment provided herein comprises or consists of K166, I168, H167, S217, and D118. In some embodiments, the epitope for the anti-CD30L antibody or antigen binding fragment provided herein comprises or consists of K166, K169, D234, H167, and D118. In some embodiments, the epitope for the anti-CD30L antibody or antigen binding fragment provided herein comprises or consists of K166, K169, D234, S217, and D118. In some embodiments, the epitope for the anti-CD30L antibody or antigen binding fragment provided herein comprises or consists of K166, K169, H167, S217, and D118. In some embodiments, the epitope for the anti-CD30L antibody or antigen binding fragment provided herein comprises or consists of K166, D234, H167, S217, and D118. In some embodiments, the epitope for the anti-CD30L antibody or antigen binding fragment provided herein comprises or consists of I168, K169, D234, H167, and D118. In some embodiments, the epitope for the anti-CD30L antibody or antigen binding fragment provided herein comprises or consists of I168, K169, D234, S217, and D118. In some embodiments, the epitope for the anti-CD30L antibody or antigen binding fragment provided herein comprises or consists of I168, K169, H167, S217, and D118. In some embodiments, the epitope for the anti-CD30L antibody or antigen binding fragment provided herein comprises or consists of I168, D234, H167, S217, and D118. In some embodiments, the epitope for the anti-CD30L antibody or antigen binding fragment provided herein comprises or consists of K169, D234, H167, S217, and D118. In some embodiments, the epitope for the anti-CD30L antibody or antigen binding fragment provided herein comprises or consists of N165, K166, I168, K169, D234, and H167. In certain embodiments, the epitope for the anti-CD30L antibody or antigen binding fragment provided herein comprises or consists of N165, K166, I168, K169, D234, and S217. In some embodiments, the epitope for the anti-CD30L antibody or antigen binding fragment provided herein comprises or consists of N165, K166, I168, K169, H167, and S217. In certain embodiments, the epitope for the anti-CD30L antibody or antigen binding fragment provided herein comprises or consists of N165, K166, I168, D234, H167, and S217. In some embodiments, the epitope for the anti-CD30L antibody or antigen binding fragment provided herein comprises or consists of N165, K166, K169, D234, H167, and S217. In certain embodiments, the epitope for the anti-CD30L antibody or antigen binding fragment provided herein comprises or consists of N165, I168, K169, D234, H167, and S217. In some embodiments, the epitope for the anti-CD30L antibody or antigen binding fragment provided herein comprises or consists of K166, I168, K169, D234, H167, and S217. In certain embodiments, the epitope for the anti-CD30L antibody or antigen binding fragment provided herein comprises or consists of N165, K166, I168, K169, D234, and D118. In some embodiments, the epitope for the anti-CD30L antibody or antigen binding fragment provided herein comprises or consists of N165, K166, I168, K169, H167, and D118. In certain embodiments, the epitope for the anti-CD30L antibody or antigen binding fragment provided herein comprises or consists of N165, K166, I168, K169, S217, and D118. In some embodiments, the epitope for the anti-CD30L antibody or antigen binding fragment provided herein comprises or consists of N165, K166, I168, D234, H167, and D118. In certain embodiments, the epitope for the anti-CD30L antibody or antigen binding fragment provided herein comprises or consists of N165, K166, I168, D234, S217, and D118. In some embodiments, the epitope for the anti-CD30L antibody or antigen binding fragment provided herein comprises or consists of N165, K166, I168, H167, S217, and D118. In certain embodiments, the epitope for the anti-CD30L antibody or antigen binding fragment provided herein comprises or consists of N165, K166, K169, D234, H167, and D118. In some embodiments, the epitope for the anti-CD30L antibody or antigen binding fragment provided herein comprises or consists of N165, K166, K169, D234, S217, and D118. In certain embodiments, the epitope for the anti-CD30L antibody or antigen binding fragment provided herein comprises or consists of N165, K166, K169, H167, S217, and D118. In some embodiments, the epitope for the anti-CD30L antibody or antigen binding fragment provided herein comprises or consists of N165, K166, D234, H167, S217, and D118. In certain embodiments, the epitope for the anti-CD30L antibody or antigen binding fragment provided herein comprises or consists of N165, I168, K169, D234, H167, and D118. In some embodiments, the epitope for the anti-CD30L antibody or antigen binding fragment provided herein comprises or consists of N165, I168, K169, D234, S217, and D118. In certain embodiments, the epitope for the anti-CD30L antibody or antigen binding fragment provided herein comprises or consists of N165, I168, K169, H167, S217, and D118. In some embodiments, the epitope for the anti-CD30L antibody or antigen binding fragment provided herein comprises or consists of N165, I168, D234, H167, S217, and D118. In certain embodiments, the epitope for the anti-CD30L antibody or antigen binding fragment provided herein comprises or consists of N165, K169, D234, H167, S217, and D118. In some embodiments, the epitope for the anti-CD30L antibody or antigen binding fragment provided herein comprises or consists of K166, I168, K169, D234, H167, and D118. In certain embodiments, the epitope for the anti-CD30L antibody or antigen binding fragment provided herein comprises or consists of K166, I168, K169, D234, S217, and D118. In some embodiments, the epitope for the anti-CD30L antibody or antigen binding fragment provided herein comprises or consists of K166, I168, K169, H167, S217, and D118. In some embodiments, the epitope for the anti-CD30L antibody or antigen binding fragment provided herein comprises or consists of K166, I168, D234, H167, S217, and D118. In certain embodiments, the epitope for the anti-CD30L antibody or antigen binding fragment provided herein comprises or consists of K166, K169, D234, H167, S217, and D118. In some embodiments, the epitope for the anti-CD30L antibody or antigen binding fragment provided herein comprises or consists of I168, K169, D234, H167, S217, and D118. In some embodiments, the epitope for the anti-CD30L antibody or antigen binding fragment provided herein comprises or consists of any one of N165, K166, I168, K169, D234, H167, and S217. In certain embodiments, the epitope for the anti-CD30L antibody or antigen binding fragment provided herein comprises or consists of any one of N165, K166, I168, K169, D234, H167, and D118. In some embodiments, the epitope for the anti-CD30L antibody or antigen binding fragment provided herein comprises or consists of any one of N165, K166, I168, K169, D234, S217, and D118. In some embodiments, the epitope for the anti-CD30L antibody or antigen binding fragment provided herein comprises or consists of any one of N165, K166, I168, K169, H167, S217, and D118. In some embodiments, the epitope for the anti-CD30L antibody or antigen binding fragment provided herein comprises or consists of any one of N165, K166, I168, D234, H167, S217, and D118. In some embodiments, the epitope for the anti-CD30L antibody or antigen binding fragment provided herein comprises or consists of any one of N165, K166, K169, D234, H167, S217, and D118. In some embodiments, the epitope for the anti-CD30L antibody or antigen binding fragment provided herein comprises or consists of any one of N165, I168, K169, D234, H167, S217, and D118. In some embodiments, the epitope for the anti-CD30L antibody or antigen binding fragment provided herein comprises or consists of any one of K166, I168, K169, D234, H167, S217, and D118. In some embodiments, the epitope for the anti-CD30L antibody or antigen binding fragment provided herein comprises or consists of any two of N165, K166, I168, K169, D234, H167, S217, and D118 in any combination or permutation. In some embodiments, the epitope for the anti-CD30L antibody or antigen binding fragment provided herein comprises or consists of any three of N165, K166, I168, K169, D234, H167, S217, and D118 in any combination or permutation. In some embodiments, the epitope for the anti-CD30L antibody or antigen binding fragment provided herein comprises or consists of any four of N165, K166, I168, K169, D234, H167, S217, and D118, in any combination or permutation. In some embodiments, the epitope for the anti-CD30L antibody or antigen binding fragment provided herein comprises or consists of any five of N165, K166, I168, K169, D234, H167, S217, and D118, in any combination or permutation. In some embodiments, the epitope for the anti-CD30L antibody or antigen binding fragment provided herein comprises or consists of any six of N165, K166, I168, K169, D234, H167, S217, and D118, in any combination or permutation. In some embodiments, the epitope for the anti-CD30L antibody or antigen binding fragment provided herein comprises or consists of seven of N165, K166, I168, K169, D234, H167, S217, and D118, in any combination or permutation. In some embodiments, the epitope for the anti-CD30L antibody or antigen binding fragment provided herein comprises or consists of eight of N165, K166, I168, K169, D234, H167, S217, and D118, in any permutation. The position of the amino acids in this paragraph are numbered according to the amino acid sequence of CD30L as set forth in SEQ ID NO: 33.

In some embodiments of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in any one of SEQ ID NOs: 110-119, 629, 636, and 712-723; (b) a CDR-H2 comprising the amino acid sequence set forth in any one of SEQ ID NOs: 150-159, 643, 650, and 724-735; (c) a CDR-H3 comprising the amino acid sequence set forth in any one of SEQ ID NOs: 190-199, 657, 664, and 736-743; (d) a CDR-L1 comprising the amino acid sequence set forth in any one of SEQ ID NOs: 310-319, 671, 678, and 744-751; (e) a CDR-L2 comprising the amino acid sequence set forth in any one of SEQ ID NOs: 350-359, 685, 692, and 752-759; and/or (f) a CDR-L3 comprising the amino acid sequence set forth in any one of SEQ ID NOs: 390-399, 699, 706, and 760-765.

In certain embodiments of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in any one of SEQ ID NOs: 110-114, 629, 636, and 712-723; (b) a CDR-H2 comprising the amino acid sequence set forth in any one of SEQ ID NOs: 150-154, 643, 650, and 724-735; (c) a CDR-H3 comprising the amino acid sequence set forth in any one of SEQ ID NOs: 190-194, 657, 664, and 736-743; (d) a CDR-L1 comprising the amino acid sequence set forth in any one of SEQ ID NOs: 310-314, 671, 678, and 744-751; (e) a CDR-L2 comprising the amino acid sequence set forth in any one of SEQ ID NOs: 350-354, 685, 692, and 752-759; and/or (f) a CDR-L3 comprising the amino acid sequence set forth in any one of SEQ ID NOs: 390-394, 699, 706, and 760-765.

In other embodiments of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in any one of SEQ ID NOs: 115-119, 629, 636, and 712-723; (b) a CDR-H2 comprising the amino acid sequence set forth in any one of SEQ ID NOs: 155-159, 643, 650, and 724-735; (c) a CDR-H3 comprising the amino acid sequence set forth in any one of SEQ ID NOs: 195-199, 657, 664, and 736-743; (d) a CDR-L1 comprising the amino acid sequence set forth in any one of SEQ ID NOs: 315-319, 671, 678, and 744-751; (e) a CDR-L2 comprising the amino acid sequence set forth in any one of SEQ ID NOs: 355-359, 685, 692, and 752-759; and/or (f) a CDR-L3 comprising the amino acid sequence set forth in any one of SEQ ID NOs: 395-399, 699, 706, and 760-765.

In some embodiments of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in any one of SEQ ID NOs: 120-129, and 712-723; (b) a CDR-H2 comprising the amino acid sequence set forth in any one of SEQ ID NOs: 160-169, and 724-735; (c) a CDR-H3 comprising the amino acid sequence set forth in any one of SEQ ID NOs: 200-209, and 736-743; (d) a CDR-L1 comprising the amino acid sequence set forth in any one of SEQ ID NOs: 320-329, and 744-751; (e) a CDR-L2 comprising the amino acid sequence set forth in any one of SEQ ID NOs: 360-369, and 752-759; and/or (f) a CDR-L3 comprising the amino acid sequence set forth in any one of SEQ ID NOs: 400-409, and 760-765.

In certain embodiments of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in any one of SEQ ID NOs: 120-124, and 712-723; (b) a CDR-H2 comprising the amino acid sequence set forth in any one of SEQ ID NOs: 160-164, and 724-735; (c) a CDR-H3 comprising the amino acid sequence set forth in any one of SEQ ID NOs: 200-204, and 736-743; (d) a CDR-L1 comprising the amino acid sequence set forth in any one of SEQ ID NOs: 320-324, and 744-751; (e) a CDR-L2 comprising the amino acid sequence set forth in any one of SEQ ID NOs: 360-364, and 752-759; and/or (f) a CDR-L3 comprising the amino acid sequence set forth in any one of SEQ ID NOs: 400-404, and 760-765.

In other embodiments of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in any one of SEQ ID NOs: 125-129, and 712-723; (b) a CDR-H2 comprising the amino acid sequence set forth in any one of SEQ ID NOs: 165-169, and 724-735; (c) a CDR-H3 comprising the amino acid sequence set forth in any one of SEQ ID NOs: 205-209, and 736-743; (d) a CDR-L1 comprising the amino acid sequence set forth in any one of SEQ ID NOs: 325-329, and 744-751; (e) a CDR-L2 comprising the amino acid sequence set forth in any one of SEQ ID NOs: 365-369, and 752-759; and/or (f) a CDR-L3 comprising the amino acid sequence set forth in any one of SEQ ID NOs: 405-409, and 760-765.

In some embodiments of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in any one of SEQ ID NOs: 130-139, and 712-723; (b) a CDR-H2 comprising the amino acid sequence set forth in any one of SEQ ID NOs: 170-179, and 724-735; (c) a CDR-H3 comprising the amino acid sequence set forth in any one of SEQ ID NOs: 210-219, and 736-743; (d) a CDR-L1 comprising the amino acid sequence set forth in any one of SEQ ID NOs: 330-339, and 744-751; (e) a CDR-L2 comprising the amino acid sequence set forth in any one of SEQ ID NOs: 370-379, and 752-759; and/or (f) a CDR-L3 comprising the amino acid sequence set forth in any one of SEQ ID NOs: 410-419, and 760-765.

In certain embodiments of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in any one of SEQ ID NOs: 130-134, and 712-723; (b) a CDR-H2 comprising the amino acid sequence set forth in any one of SEQ ID NOs: 170-174, and 724-735; (c) a CDR-H3 comprising the amino acid sequence set forth in any one of SEQ ID NOs: 210-214, and 736-743; (d) a CDR-L1 comprising the amino acid sequence set forth in any one of SEQ ID NOs: 330-334, and 744-751; (e) a CDR-L2 comprising the amino acid sequence set forth in any one of SEQ ID NOs: 370-374, and 752-759; and/or (f) a CDR-L3 comprising the amino acid sequence set forth in any one of SEQ ID NOs: 410-414, and 760-765.

In other embodiments of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in any one of SEQ ID NOs: 135-139, and 712-723; (b) a CDR-H2 comprising the amino acid sequence set forth in any one of SEQ ID NOs: 175-179, and 724-735; (c) a CDR-H3 comprising the amino acid sequence set forth in any one of SEQ ID NOs: 215-219, and 736-743; (d) a CDR-L1 comprising the amino acid sequence set forth in any one of SEQ ID NOs: 335-339, and 744-751; (e) a CDR-L2 comprising the amino acid sequence set forth in any one of SEQ ID NOs: 375-379, and 752-759; and/or (f) a CDR-L3 comprising the amino acid sequence set forth in any one of SEQ ID NOs: 415-419, and 760-765.

In some embodiments of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in any one of SEQ ID NOs: 220-224, and 712-723; (b) a CDR-H2 comprising the amino acid sequence set forth in any one of SEQ ID NOs: 235-239, and 724-735; (c) a CDR-H3 comprising the amino acid sequence set forth in any one of SEQ ID NOs: 250-254, and 736-743; (d) a CDR-L1 comprising the amino acid sequence set forth in any one of SEQ ID NOs: 420-424, and 744-751; (e) a CDR-L2 comprising the amino acid sequence set forth in any one of SEQ ID NOs: 435-439, and 752-759; and/or (f) a CDR-L3 comprising the amino acid sequence set forth in any one of SEQ ID NOs: 450-454, and 760-765.

In certain embodiments of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in any one of SEQ ID NOs: 225-229, and 712-723; (b) a CDR-H2 comprising the amino acid sequence set forth in any one of SEQ ID NOs: 240-244, and 724-735; (c) a CDR-H3 comprising the amino acid sequence set forth in any one of SEQ ID NOs: 255-259, and 736-743; (d) a CDR-L1 comprising the amino acid sequence set forth in any one of SEQ ID NOs: 425-429, and 744-751; (e) a CDR-L2 comprising the amino acid sequence set forth in any one of SEQ ID NOs: 440-444, and 752-759; and/or (f) a CDR-L3 comprising the amino acid sequence set forth in any one of SEQ ID NOs: 455-459, and 760-765.

In other embodiments of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in any one of SEQ ID NOs: 230-234, and 712-723; (b) a CDR-H2 comprising the amino acid sequence set forth in any one of SEQ ID NOs: 245-249, and 724-735; (c) a CDR-H3 comprising the amino acid sequence set forth in any one of SEQ ID NOs: 260-264, and 736-743; (d) a CDR-L1 comprising the amino acid sequence set forth in any one of SEQ ID NOs: 430-434, and 744-751; (e) a CDR-L2 comprising the amino acid sequence set forth in any one of SEQ ID NOs: 445-449, and 752-759; and/or (f) a CDR-L3 comprising the amino acid sequence set forth in any one of SEQ ID NOs: 460-464, and 760-765.

In one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in any one of SEQ ID NOs: 465-469, 631, 638, and 712-723; (b) a CDR-H2 comprising the amino acid sequence set forth in any one of SEQ ID NOs: 490-494, 645, 652, and 724-735; (c) a CDR-H3 comprising the amino acid sequence set forth in any one of SEQ ID NOs: 528-532, 659, 666, and 736-743; (d) a CDR-L1 comprising the amino acid sequence set forth in any one of SEQ ID NOs: 553-557, 673, 680, and 744-751; (e) a CDR-L2 comprising the amino acid sequence set forth in any one of SEQ ID NOs: 578-582, 687, 694, and 752-759; and/or (f) a CDR-L3 comprising the amino acid sequence set forth in any one of SEQ ID NOs: 603-607, 701, 708, and 760-765;
In another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in any one of SEQ ID NOs: 470-474, 632, 639, and 712-723; (b) a CDR-H2 comprising the amino acid sequence set forth in any one of SEQ ID NOs: 495-499, 646, 653, and 724-735; (c) a CDR-H3 comprising the amino acid sequence set forth in any one of SEQ ID NOs: 533-537, 660, 667, and 736-743; (d) a CDR-L1 comprising the amino acid sequence set forth in any one of SEQ ID NOs: 558-562, 674, 681, and 744-751; (e) a CDR-L2 comprising the amino acid sequence set forth in any one of SEQ ID NOs: 583-587, 688, 695, and 752-759; and/or (f) a CDR-L3 comprising the amino acid sequence set forth in any one of SEQ ID NOs: 608-612, 702, 709, and 760-765;
In a further embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in any one of SEQ ID NOs: 475-479, 633, 640, and 712-723; (b) a CDR-H2 comprising the amino acid sequence set forth in any one of SEQ ID NOs: 513-517, 647, 654, and 724-735; (c) a CDR-H3 comprising the amino acid sequence set forth in any one of SEQ ID NOs: 538-542, 661, 668, and 736-743; (d) a CDR-L1 comprising the amino acid sequence set forth in any one of SEQ ID NOs: 563-567, 675, 682, and 744-751; (e) a CDR-L2 comprising the amino acid sequence set forth in any one of SEQ ID NOs: 588-592, 689, 696, and 752-759; and/or (f) a CDR-L3 comprising the amino acid sequence set forth in any one of SEQ ID NOs: 613-617, 703, 710, and 760-765;
In yet another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in any one of SEQ ID NOs: 480-484, 630, 637, and 712-723; (b) a CDR-H2 comprising the amino acid sequence set forth in any one of SEQ ID NOs: 518-522, 644, 651, and 724-735; (c) a CDR-H3 comprising the amino acid sequence set forth in any one of SEQ ID NOs: 543-547, 658, 665, and 736-743; (d) a CDR-L1 comprising the amino acid sequence set forth in any one of SEQ ID NOs: 568-572, 672, 679, and 744-751; (e) a CDR-L2 comprising the amino acid sequence set forth in any one of SEQ ID NOs: 593-597, 686, 693, and 752-759; and/or (f) a CDR-L3 comprising the amino acid sequence set forth in any one of SEQ ID NOs: 618-622, 700, 707, and 760-765; or
In one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in any one of SEQ ID Nos: 485-489, 634, 641, and 712-723; (b) a CDR-H2 comprising the amino acid sequence set forth in any one of SEQ ID Nos: 523-527, 648, 655, and 724-735; (c) a CDR-H3 comprising the amino acid sequence set forth in any one of SEQ ID Nos: 548-552, 662, 669, and 736-743; (d) a CDR-L1 comprising the amino acid sequence set forth in any one of SEQ ID Nos: 573-577, 676, 683, and 744-751; € a CDR-L2 comprising the amino acid sequence set forth in any one of SEQ ID Nos: 598-602, 690, 697, and 752-759; and/or (f) a CDR-L3 comprising the amino acid sequence set forth in any one of SEQ ID Nos: 623-627, 704, 711, and 760-765.

The disclosure further provides the sequence and structural features in the CDR sequences common to the antibodies that confer the anti-CD30L antibody the binding affinity, specificity, and the capacity to functionally block both the CD30-CD30L interaction and the CD30L-mediated CD30 signaling and IL-8 releasing. Accordingly, also provided herein are anti-CD30L antibodies with consensus CDR sequences that confer the anti-CD30L antibody the binding affinity, specificity, and the capacity to functionally block both the CD30-CD30L interaction and the CD30L-mediated CD30 signaling and IL-8 releasing. In one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in any one of SEQ ID NOs: 712-723; (b) a CDR-H2 comprising the amino acid sequence set forth in any one of SEQ ID NOs: 724-735; (c) a CDR-H3 comprising the amino acid sequence set forth in any one of SEQ ID NOs: 736-743; (d) a CDR-L1 comprising the amino acid sequence set forth in any one of SEQ ID NOs: 744-751; (e) a CDR-L2 comprising the amino acid sequence set forth in any one of SEQ ID NOs: 752-759; and/or (f) a CDR-L3 comprising the amino acid sequence set forth in any one of SEQ ID NOs: 760-765. In another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in any one of SEQ ID NOs: 712, 714, 716, 718, 720, and 722; (b) a CDR-H2 comprising the amino acid sequence set forth in any one of SEQ ID NOs: 724, 726, 728, 730, 732, and 734; (c) a CDR-H3 comprising the amino acid sequence set forth in any one of SEQ ID NOs: 736, 738, 740, and 742; (d) a CDR-L1 comprising the amino acid sequence set forth in any one of SEQ ID NOs: 744, 746, 748, and 750; (e) a CDR-L2 comprising the amino acid sequence set forth in any one of SEQ ID NOs: 752, 754, 756, and 758; and/or (f) a CDR-L3 comprising the amino acid sequence set forth in any one of SEQ ID NOs: 760, 762, and 764. In a further embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in any one of SEQ ID NOs: 713, 715, 717, 719, 721, and 723; (b) a CDR-H2 comprising the amino acid sequence set forth in any one of SEQ ID NOs: 725, 727, 729, 731, 733, and 735; (c) a CDR-H3 comprising the amino acid sequence set forth in any one of SEQ ID NOs: 737, 739, 741, and 743; (d) a CDR-L1 comprising the amino acid sequence set forth in any one of SEQ ID NOs: 745, 747, 749, and 751; (e) a CDR-L2 comprising the amino acid sequence set forth in any one of SEQ ID NOs: 753, 755, 757, and 759; and/or (f) a CDR-L3 comprising the amino acid sequence set forth in any one of SEQ ID NOs: 761, 763, and 765. In yet another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 712; (b) a CDR-H2 comprising the amino acid sequence set forth in SEQ ID NO: 730; (c) a CDR-H3 comprising the amino acid sequence set forth in SEQ ID NO: 736; (d) a CDR-L1 comprising the amino acid sequence set forth in SEQ ID NO: 744; (e) a CDR-L2 comprising the amino acid sequence set forth SEQ ID NO: 752; and/or (f) a CDR-L3 comprising the amino acid sequence set forth SEQ ID NO: 760. In one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 713; (b) a CDR-H2 comprising the amino acid sequence set forth in SEQ ID NO: 731; (c) a CDR-H3 comprising the amino acid sequence set forth in SEQ ID NO: 737; (d) a CDR-L1 comprising the amino acid sequence set forth in SEQ ID NO: 745; (e) a CDR-L2 comprising the amino acid sequence set forth SEQ ID NO: 753; and/or (f) a CDR-L3 comprising the amino acid sequence set forth SEQ ID NO: 761. In another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 712; (b) a CDR-H2 comprising the amino acid sequence set forth in SEQ ID NO: 724; (c) a CDR-H3 comprising the amino acid sequence set forth in SEQ ID NO: 736; (d) a CDR-L1 comprising the amino acid sequence set forth in SEQ ID NO: 744; (e) a CDR-L2 comprising the amino acid sequence set forth SEQ ID NO: 752; and/or (f) a CDR-L3 comprising the amino acid sequence set forth SEQ ID NO: 760. In a further embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 713; (b) a CDR-H2 comprising the amino acid sequence set forth in SEQ ID NO: 725; (c) a CDR-H3 comprising the amino acid sequence set forth in SEQ ID NO: 737; (d) a CDR-L1 comprising the amino acid sequence set forth in SEQ ID NO: 745; (e) a CDR-L2 comprising the amino acid sequence set forth SEQ ID NO: 753; and/or (f) a CDR-L3 comprising the amino acid sequence set forth SEQ ID NO: 761. In yet another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 714; (b) a CDR-H2 comprising the amino acid sequence set forth in SEQ ID NO: 726; (c) a CDR-H3 comprising the amino acid sequence set forth in SEQ ID NO: 736; (d) a CDR-L1 comprising the amino acid sequence set forth in SEQ ID NO: 744; (e) a CDR-L2 comprising the amino acid sequence set forth SEQ ID NO: 752; and/or (f) a CDR-L3 comprising the amino acid sequence set forth SEQ ID NO: 760. In one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 715; (b) a CDR-H2 comprising the amino acid sequence set forth in SEQ ID NO: 727; (c) a CDR-H3 comprising the amino acid sequence set forth in SEQ ID NO: 737; (d) a CDR-L1 comprising the amino acid sequence set forth in SEQ ID NO: 745; (e) a CDR-L2 comprising the amino acid sequence set forth SEQ ID NO: 753; and/or (f) a CDR-L3 comprising the amino acid sequence set forth SEQ ID NO: 761. In another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 716; (b) a CDR-H2 comprising the amino acid sequence set forth in SEQ ID NO: 728; (c) a CDR-H3 comprising the amino acid sequence set forth in SEQ ID NO: 736; (d) a CDR-L1 comprising the amino acid sequence set forth in SEQ ID NO: 744; (e) a CDR-L2 comprising the amino acid sequence set forth SEQ ID NO: 752; and/or (f) a CDR-L3 comprising the amino acid sequence set forth SEQ ID NO: 760. In yet another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 717; (b) a CDR-H2 comprising the amino acid sequence set forth in SEQ ID NO: 729; (c) a CDR-H3 comprising the amino acid sequence set forth in SEQ ID NO: 737; (d) a CDR-L1 comprising the amino acid sequence set forth in SEQ ID NO: 745; (e) a CDR-L2 comprising the amino acid sequence set forth SEQ ID NO: 753; and/or (f) a CDR-L3 comprising the amino acid sequence set forth SEQ ID NO: 761. In one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 718; (b) a CDR-H2 comprising the amino acid sequence set forth in SEQ ID NO: 730; (c) a CDR-H3 comprising the amino acid sequence set forth in SEQ ID NO: 738; (d) a CDR-L1 comprising the amino acid sequence set forth in SEQ ID NO: 746; (e) a CDR-L2 comprising the amino acid sequence set forth SEQ ID NO: 754; and/or (f) a CDR-L3 comprising the amino acid sequence set forth SEQ ID NO: 762. In one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 719; (b) a CDR-H2 comprising the amino acid sequence set forth in SEQ ID NO: 731; (c) a CDR-H3 comprising the amino acid sequence set forth in SEQ ID NO: 739; (d) a CDR-L1 comprising the amino acid sequence set forth in SEQ ID NO: 747; (e) a CDR-L2 comprising the amino acid sequence set forth SEQ ID NO: 755; and/or (f) a CDR-L3 comprising the amino acid sequence set forth SEQ ID NO: 763. In another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 720; (b) a CDR-H2 comprising the amino acid sequence set forth in SEQ ID NO: 732; (c) a CDR-H3 comprising the amino acid sequence set forth in SEQ ID NO: 740; (d) a CDR-L1 comprising the amino acid sequence set forth in SEQ ID NO: 748; (e) a CDR-L2 comprising the amino acid sequence set forth SEQ ID NO: 756; and/or (f) a CDR-L3 comprising the amino acid sequence set forth SEQ ID NO: 760. In a further embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 721; (b) a CDR-H2 comprising the amino acid sequence set forth in SEQ ID NO: 733; (c) a CDR-H3 comprising the amino acid sequence set forth in SEQ ID NO: 741; (d) a CDR-L1 comprising the amino acid sequence set forth in SEQ ID NO: 749; (e) a CDR-L2 comprising the amino acid sequence set forth SEQ ID NO: 757; and/or (f) a CDR-L3 comprising the amino acid sequence set forth SEQ ID NO: 761. In a further embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 722; (b) a CDR-H2 comprising the amino acid sequence set forth in SEQ ID NO: 734; (c) a CDR-H3 comprising the amino acid sequence set forth in SEQ ID NO: 742; (d) a CDR-L1 comprising the amino acid sequence set forth in SEQ ID NO: 750; (e) a CDR-L2 comprising the amino acid sequence set forth SEQ ID NO: 758; and/or (f) a CDR-L3 comprising the amino acid sequence set forth SEQ ID NO: 764. In yet another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 723; (b) a CDR-H2 comprising the amino acid sequence set forth in SEQ ID NO: 735; (c) a CDR-H3 comprising the amino acid sequence set forth in SEQ ID NO: 743; (d) a CDR-L1 comprising the amino acid sequence set forth in SEQ ID NO: 751; (e) a CDR-L2 comprising the amino acid sequence set forth SEQ ID NO: 759; and/or (f) a CDR-L3 comprising the amino acid sequence set forth SEQ ID NO: 765. In some embodiments of the anti-CD30L provided herein including in Section 2 and this Section 4.1 (such as those of this paragraph), the CDR-H1 comprises the amino acid sequence set forth in any one of SEQ ID NOs: 105-109, 628, 635, 115-119, 629, 636, 480-484, 630, 637, 465-469, 631, 638, 470-474, 632, 639, 475-479, 633, 640, 485-489, 634, and 641. In some embodiments of the anti-CD30L provided herein including in Section 2 and this Section 4.1 (such as those of this paragraph), the CDR-H2 comprises the amino acid sequence set forth in any one of SEQ ID NOs: 145-149, 642, 649, 155-159, 643, 650, 518-522, 644, 651, 490-494, 645, 652, 495-499, 646, 653, 513-517, 647, 654, 523-527, 648, and 655. In some embodiments of the anti-CD30L provided herein including in Section 2 and this Section 4.1 (such as those of this paragraph), the CDR-H3 comprises the amino acid sequence set forth in any one of SEQ ID NOs: 185-189, 656, 663, 195-199, 657, 664, 543-547, 658, 665, 528-532, 659, 666, 533-537, 660, 667, 538-542, 661, 668, 548-552, 662, and 669. In some embodiments of the anti-CD30L provided herein including in Section 2 and this Section 4.1 (such as those of this paragraph), the CDR-L1 comprises the amino acid sequence set forth in any one of SEQ ID NOs: 305-309, 670, 677, 315-319, 671, 678, 568-572, 672, 679, 553-557, 673, 680, 558-562, 674, 681, 563-567, 675, 682, 573-577, 676, and 683. In some embodiments of the anti-CD30L provided herein including in Section 2 and this Section 4.1 (such as those of this paragraph), the CDR-L2 comprises the amino acid sequence set forth in any one of SEQ ID NOs: 345-349, 684, 691, 355-359, 685, 692, 593-597, 686, 693, 578-582, 687, 694, 583-587, 688, 695, 588-592, 689, 696, 598-602, 690, and 697. In some embodiments of the anti-CD30L provided herein including in Section 2 and this Section 4.1 (such as those of this paragraph), the CDR-L3 comprises the amino acid sequence set forth in any one of SEQ ID NOs: 385-389, 698, 705, 395-399, 699, 706, 618-622, 700, 707, 603-607, 701, 708, 608-612, 702, 709, 613-617, 703, 710, 623-627, 704, and 711. In certain embodiments of the anti-CD30L provided herein including in Section 2 and this Section 4.1 (such as those of this paragraph), the CDR-H1 comprises the amino acid sequence set forth in any one of SEQ ID NOs: 105-109, 628, 635, 115-119, 629, 636, 480-484, 630, and 637. In certain embodiments of the anti-CD30L provided herein including in Section 2 and this Section 4.1 (such as those of this paragraph), the CDR-H2 comprises the amino acid sequence set forth in any one of SEQ ID NOs: 145-149, 642, 649, 155-159, 643, 650, 518-522, 644, and 651. In certain embodiments of the anti-CD30L provided herein including in Section 2 and this Section 4.1 (such as those of this paragraph), the CDR-H3 comprises the amino acid sequence set forth in any one of SEQ ID NOs: 185-189, 656, 663, 195-199, 657, 664, 543-547, 658, and 665. In certain embodiments of the anti-CD30L provided herein including in Section 2 and this Section 4.1 (such as those of this paragraph), the CDR-L1 comprises the amino acid sequence set forth in any one of SEQ ID NOs: 305-309, 670, 677, 315-319, 671, 678, 568-572, 672, and 679. In certain embodiments of the anti-CD30L provided herein including in Section 2 and this Section 4.1 (such as those of this paragraph), the CDR-L2 comprises the amino acid sequence set forth in any one of SEQ ID NOs: 345-349, 684, 691, 355-359, 685, 692, 593-597, 686, and 693. In certain embodiments of the anti-CD30L provided herein including in Section 2 and this Section 4.1 (such as those of this paragraph), the CDR-L3 comprises the amino acid sequence set forth in any one of SEQ ID NOs: 385-389, 698, 705, 395-399, 699, 706, 618-622, 700, and 707.

Further to the anti-CD30L antibody provided herein including in Section 2 and this Section 4.1 (such as those of the preceding paragraph), in one embodiment, the anti-CD30L antibody or antigen binding fragment provided herein comprises or consists of a CDR-H1 comprising the amino acid sequence set forth in any one of SEQ ID NOs: 712-723. In some embodiments, the anti-CD30L antibody or antigen binding fragment provided herein comprises or consists of a CDR-H2 comprising the amino acid sequence set forth in any one of SEQ ID NOs: 724-735. In certain embodiments, the anti-CD30L antibody or antigen binding fragment provided herein comprises or consists of a CDR-H3 comprising the amino acid sequence set forth in any one of SEQ ID NOs: 736-743. In other embodiments, the anti-CD30L antibody or antigen binding fragment provided herein comprises or consists of a CDR-L1 comprising the amino acid sequence set forth in any one of SEQ ID NOs: 744-751. In yet other embodiments, the anti-CD30L antibody or antigen binding fragment provided herein comprises or consists of a CDR-L2 comprising the amino acid sequence set forth in any one of SEQ ID NOs: 752-759. In another embodiment, the anti-CD30L antibody or antigen binding fragment provided herein comprises or consists of a CDR-L3 comprising the amino acid sequence set forth in any one of SEQ ID NOs: 760-765. In one embodiment, the anti-CD30L antibody or antigen binding fragment provided herein comprises or consists of a CDR-H1 comprising the amino acid sequence set forth in any one of SEQ ID NOs: 712-723 and a CDR-H2 comprising the amino acid sequence set forth in any one of SEQ ID NOs: 724-735. In some embodiments, the anti-CD30L antibody or antigen binding fragment provided herein comprises or consists of a CDR-H1 comprising the amino acid sequence set forth in any one of SEQ ID NOs: 712-723 and a CDR-H3 comprising the amino acid sequence set forth in any one of SEQ ID NOs: 736-743. In certain embodiments, the anti-CD30L antibody or antigen binding fragment provided herein comprises or consists of a CDR-H1 comprising the amino acid sequence set forth in any one of SEQ ID NOs: 712-723 and a CDR-L1 comprising the amino acid sequence set forth in any one of SEQ ID NOs: 744-751. In other embodiments, the anti-CD30L antibody or antigen binding fragment provided herein comprises or consists of a CDR-H1 comprising the amino acid sequence set forth in any one of SEQ ID NOs: 712-723 and a CDR-L2 comprising the amino acid sequence set forth in any one of SEQ ID NOs: 752-759. In some embodiments, the anti-CD30L antibody or antigen binding fragment provided herein comprises or consists of a CDR-H1 comprising the amino acid sequence set forth in any one of SEQ ID NOs: 712-723 and a CDR-L3 comprising the amino acid sequence set forth in any one of SEQ ID NOs: 760-765. In further embodiments, the anti-CD30L antibody or antigen binding fragment provided herein comprises or consists of a CDR-H2 comprising the amino acid sequence set forth in any one of SEQ ID NOs: 724-735 and a CDR-H3 comprising the amino acid sequence set forth in any one of SEQ ID NOs: 736-743. In one embodiment, the anti-CD30L antibody or antigen binding fragment provided herein comprises or consists of a CDR-H2 comprising the amino acid sequence set forth in any one of SEQ ID NOs: 724-735 and a CDR-L1 comprising the amino acid sequence set forth in any one of SEQ ID NOs: 744-751. In some embodiments, the anti-CD30L antibody or antigen binding fragment provided herein comprises or consists of a CDR-H2 comprising the amino acid sequence set forth in any one of SEQ ID NOs: 724-735 and a CDR-L2 comprising the amino acid sequence set forth in any one of SEQ ID NOs: 752-759. In certain embodiments, the anti-CD30L antibody or antigen binding fragment provided herein comprises or consists of a CDR-H2 comprising the amino acid sequence set forth in any one of SEQ ID NOs: 724-735 and a CDR-L3 comprising the amino acid sequence set forth in any one of SEQ ID NOs: 760-765. In other embodiments, the anti-CD30L antibody or antigen binding fragment provided herein comprises or consists of a CDR-H3 comprising the amino acid sequence set forth in any one of SEQ ID NOs: 736-743 and a CDR-L1 comprising the amino acid sequence set forth in any one of SEQ ID NOs: 744-751. In yet other embodiments, the anti-CD30L antibody or antigen binding fragment provided herein comprises or consists of a CDR-H3 comprising the amino acid sequence set forth in any one of SEQ ID NOs: 736-743 and a CDR-L2 comprising the amino acid sequence set forth in any one of SEQ ID NOs: 752-759. In some embodiments, the anti-CD30L antibody or antigen binding fragment provided herein comprises or consists of a CDR-H3 comprising the amino acid sequence set forth in any one of SEQ ID NOs: 736-743 and a CDR-L3 comprising the amino acid sequence set forth in any one of SEQ ID NOs: 760-765. In one embodiment, the anti-CD30L antibody or antigen binding fragment provided herein comprises or consists of a CDR-L1 comprising the amino acid sequence set forth in any one of SEQ ID NOs: 744-751 and a CDR-L2 comprising the amino acid sequence set forth in any one of SEQ ID NOs: 752-759. In another embodiment, the anti-CD30L antibody or antigen binding fragment provided herein comprises or consists of a CDR-L1 comprising the amino acid sequence set forth in any one of SEQ ID NOs: 744-751 and a CDR-L3 comprising the amino acid sequence set forth in any one of SEQ ID NOs: 760-765. In another embodiment, the anti-CD30L antibody or antigen binding fragment provided herein comprises or consists of a CDR-L2 comprising the amino acid sequence set forth in any one of SEQ ID NOs: 752-759 and a CDR-L3 comprising the amino acid sequence set forth in any one of SEQ ID NOs: 760-765. In other embodiments, the anti-CD30L antibody or antigen binding fragment provided herein comprises or consists of a CDR-H1 comprising the amino acid sequence set forth in any one of SEQ ID NOs: 712-723, a CDR-H2 comprising the amino acid sequence set forth in any one of SEQ ID NOs: 724-735, and a CDR-H3 comprising the amino acid sequence set forth in any one of SEQ ID NOs: 736-743. In yet other embodiments, the anti-CD30L antibody or antigen binding fragment provided herein comprises or consists of a CDR-H1 comprising the amino acid sequence set forth in any one of SEQ ID NOs: 712-723, a CDR-H2 comprising the amino acid sequence set forth in any one of SEQ ID NOs: 724-735 and a CDR-L1 comprising the amino acid sequence set forth in any one of SEQ ID NOs: 744-751. In further embodiments, the anti-CD30L antibody or antigen binding fragment provided herein comprises or consists of a CDR-H1 comprising the amino acid sequence set forth in any one of SEQ ID NOs: 712-723, a CDR-H2 comprising the amino acid sequence set forth in any one of SEQ ID NOs: 724-735, and a CDR-L2 comprising the amino acid sequence set forth in any one of SEQ ID NOs: 752-759. In some embodiments, the anti-CD30L antibody or antigen binding fragment provided herein comprises or consists of a CDR-H1 comprising the amino acid sequence set forth in any one of SEQ ID NOs: 712-723, a CDR-H2 comprising the amino acid sequence set forth in any one of SEQ ID NOs: 724-735, and a CDR-L3 comprising the amino acid sequence set forth in any one of SEQ ID NOs: 760-765. In some embodiments, the anti-CD30L antibody or antigen binding fragment provided herein comprises or consists of a CDR-H1 comprising the amino acid sequence set forth in any one of SEQ ID NOs: 712-723, a CDR-H3 comprising the amino acid sequence set forth in any one of SEQ ID NOs: 736-743 and a CDR-L1 comprising the amino acid sequence set forth in any one of SEQ ID NOs: 744-751. In certain embodiments, the anti-CD30L antibody or antigen binding fragment provided herein comprises or consists of a CDR-H1 comprising the amino acid sequence set forth in any one of SEQ ID NOs: 712-723, a CDR-H3 comprising the amino acid sequence set forth in any one of SEQ ID NOs: 736-743, and a CDR-L2 comprising the amino acid sequence set forth in any one of SEQ ID NOs: 752-759. In some embodiments, the anti-CD30L antibody or antigen binding fragment provided herein comprises or consists of a CDR-H1 comprising the amino acid sequence set forth in any one of SEQ ID NOs: 712-723, a CDR-H3 comprising the amino acid sequence set forth in any one of SEQ ID NOs: 736-743, and a CDR-L3 comprising the amino acid sequence set forth in any one of SEQ ID NOs: 760-765. In yet other embodiments, the anti-CD30L antibody or antigen binding fragment provided herein comprises or consists of a CDR-H1 comprising the amino acid sequence set forth in any one of SEQ ID NOs: 712-723, a CDR-L1 comprising the amino acid sequence set forth in any one of SEQ ID NOs: 744-751 and a CDR-L2 comprising the amino acid sequence set forth in any one of SEQ ID NOs: 752-759. In some embodiments, the anti-CD30L antibody or antigen binding fragment provided herein comprises or consists of a CDR-H1 comprising the amino acid sequence set forth in any one of SEQ ID NOs: 712-723, a CDR-L1 comprising the amino acid sequence set forth in any one of SEQ ID NOs: 744-751 and a CDR-L3 comprising the amino acid sequence set forth in any one of SEQ ID NOs: 760-765. In some embodiments, the anti-CD30L antibody or antigen binding fragment provided herein comprises or consists of a CDR-H1 comprising the amino acid sequence set forth in any one of SEQ ID NOs: 712-723, a CDR-L2 comprising the amino acid sequence set forth in any one of SEQ ID NOs: 752-759 and a CDR-L3 comprising the amino acid sequence set forth in any one of SEQ ID NOs: 760-765. In some embodiments, the anti-CD30L antibody or antigen binding fragment provided herein comprises or consists of a CDR-H2 comprising the amino acid sequence set forth in any one of SEQ ID NOs: 724-735, a CDR-H3 comprising the amino acid sequence set forth in any one of SEQ ID NOs: 736-743 and a CDR-L1 comprising the amino acid sequence set forth in any one of SEQ ID NOs: 744-751. In certain embodiments, the anti-CD30L antibody or antigen binding fragment provided herein comprises or consists of a CDR-H2 comprising the amino acid sequence set forth in any one of SEQ ID NOs: 724-735, a CDR-H3 comprising the amino acid sequence set forth in any one of SEQ ID NOs: 736-743, and a CDR-L2 comprising the amino acid sequence set forth in any one of SEQ ID NOs: 752-759. In some embodiments, the anti-CD30L antibody or antigen binding fragment provided herein comprises or consists of a CDR-H2 comprising the amino acid sequence set forth in any one of SEQ ID NOs: 724-735, a CDR-H3 comprising the amino acid sequence set forth in any one of SEQ ID NOs: 736-743, and a CDR-L3 comprising the amino acid sequence set forth in any one of SEQ ID NOs: 760-765. In other embodiments, the anti-CD30L antibody or antigen binding fragment provided herein comprises or consists of a CDR-H2 comprising the amino acid sequence set forth in any one of SEQ ID NOs: 724-735, a CDR-L1 comprising the amino acid sequence set forth in any one of SEQ ID NOs: 744-751 and a CDR-L2 comprising the amino acid sequence set forth in any one of SEQ ID NOs: 752-759. In yet other embodiments, the anti-CD30L antibody or antigen binding fragment provided herein comprises or consists of a CDR-H2 comprising the amino acid sequence set forth in any one of SEQ ID NOs: 724-735, a CDR-L1 comprising the amino acid sequence set forth in any one of SEQ ID NOs: 744-751 and a CDR-L3 comprising the amino acid sequence set forth in any one of SEQ ID NOs: 760-765. In some embodiments, the anti-CD30L antibody or antigen binding fragment provided herein comprises or consists of a CDR-H2 comprising the amino acid sequence set forth in any one of SEQ ID NOs: 724-735, a CDR-L2 comprising the amino acid sequence set forth in any one of SEQ ID NOs: 752-759 and a CDR-L3 comprising the amino acid sequence set forth in any one of SEQ ID NOs: 760-765. In yet other embodiments, the anti-CD30L antibody or antigen binding fragment provided herein comprises or consists of a CDR-H3 comprising the amino acid sequence set forth in any one of SEQ ID NOs: 736-743, a CDR-L1 comprising the amino acid sequence set forth in any one of SEQ ID NOs: 744-751 and a CDR-L2 comprising the amino acid sequence set forth in any one of SEQ ID NOS: 752-759. In other embodiments, the anti-CD30L antibody or antigen binding fragment provided herein comprises or consists of a CDR-H3 comprising the amino acid sequence set forth in any one of SEQ ID NOs: 736-743, a CDR-L1 comprising the amino acid sequence set forth in any one of SEQ ID NOs: 744-751 and a CDR-L3 comprising the amino acid sequence set forth in any one of SEQ ID NOs: 760-765. In certain embodiments, the anti-CD30L antibody or antigen binding fragment provided herein comprises or consists of a CDR-H3 comprising the amino acid sequence set forth in any one of SEQ ID NOs: 736-743, a CDR-L2 comprising the amino acid sequence set forth in any one of SEQ ID NOs: 752-759 and a CDR-L3 comprising the amino acid sequence set forth in any one of SEQ ID NOs: 760-765. In some embodiments, the anti-CD30L antibody or antigen binding fragment provided herein comprises or consists of a CDR-L1 comprising the amino acid sequence set forth in any one of SEQ ID NOs: 744-751, a CDR-L2 comprising the amino acid sequence set forth in any one of SEQ ID NOs: 752-759 and a CDR-L3 comprising the amino acid sequence set forth in any one of SEQ ID NOs: 760-765. In other embodiments, the anti-CD30L antibody or antigen binding fragment provided herein comprises or consists of a CDR-H1 comprising the amino acid sequence set forth in any one of SEQ ID NOs: 712-723, a CDR-H2 comprising the amino acid sequence set forth in any one of SEQ ID NOs: 724-735, a CDR-H3 comprising the amino acid sequence set forth in any one of SEQ ID NOs: 736-743, and a CDR-L1 comprising the amino acid sequence set forth in any one of SEQ ID NOs: 744-751. In further embodiments, the anti-CD30L antibody or antigen binding fragment provided herein comprises or consists of a CDR-H1 comprising the amino acid sequence set forth in any one of SEQ ID NOs: 712-723, a CDR-H2 comprising the amino acid sequence set forth in any one of SEQ ID NOs: 724-735, a CDR-H3 comprising the amino acid sequence set forth in any one of SEQ ID NOs: 736-743, and a CDR-L2 comprising the amino acid sequence set forth in any one of SEQ ID NOs: 752-759. In some embodiments, the anti-CD30L antibody or antigen binding fragment provided herein comprises or consists of a CDR-H1 comprising the amino acid sequence set forth in any one of SEQ ID NOs: 712-723, a CDR-H2 comprising the amino acid sequence set forth in any one of SEQ ID NOs: 724-735, a CDR-H3 comprising the amino acid sequence set forth in any one of SEQ ID NOs: 736-743, and a CDR-L3 comprising the amino acid sequence set forth in any one of SEQ ID NOs: 760-765. In some embodiments, the anti-CD30L antibody or antigen binding fragment provided herein comprises or consists of a CDR-H1 comprising the amino acid sequence set forth in any one of SEQ ID NOs: 712-723, a CDR-H2 comprising the amino acid sequence set forth in any one of SEQ ID NOs: 724-735, a CDR-L1 comprising the amino acid sequence set forth in any one of SEQ ID NOs: 744-751, and a CDR-L2 comprising the amino acid sequence set forth in any one of SEQ ID NOs: 752-759. In some embodiments, the anti-CD30L antibody or antigen binding fragment provided herein comprises or consists of a CDR-H1 comprising the amino acid sequence set forth in any one of SEQ ID NOs: 712-723, a CDR-H2 comprising the amino acid sequence set forth in any one of SEQ ID NOs: 724-735, a CDR-L1 comprising the amino acid sequence set forth in any one of SEQ ID NOs: 744-751, and a CDR-L3 comprising the amino acid sequence set forth in any one of SEQ ID NOs: 760-765. In some embodiments, the anti-CD30L antibody or antigen binding fragment provided herein comprises or consists of a CDR-H1 comprising the amino acid sequence set forth in any one of SEQ ID NOs: 712-723, a CDR-H2 comprising the amino acid sequence set forth in any one of SEQ ID NOs: 724-735, a CDR-L2 comprising the amino acid sequence set forth in any one of SEQ ID NOs: 752-759, and a CDR-L3 comprising the amino acid sequence set forth in any one of SEQ ID NOs: 760-765. In certain embodiments, the anti-CD30L antibody or antigen binding fragment provided herein comprises or consists of a CDR-H1 comprising the amino acid sequence set forth in any one of SEQ ID NOs: 712-723, a CDR-H3 comprising the amino acid sequence set forth in any one of SEQ ID NOs: 736-743, a CDR-L1 comprising the amino acid sequence set forth in any one of SEQ ID NOs: 744-751, and a CDR-L2 comprising the amino acid sequence set forth in any one of SEQ ID NOs: 752-759. In certain embodiments, the anti-CD30L antibody or antigen binding fragment provided herein comprises or consists of a CDR-H1 comprising the amino acid sequence set forth in any one of SEQ ID NOs: 712-723, a CDR-H3 comprising the amino acid sequence set forth in any one of SEQ ID NOs: 736-743, a CDR-L1 comprising the amino acid sequence set forth in any one of SEQ ID NOs: 744-751, and a CDR-L3 comprising the amino acid sequence set forth in any one of SEQ ID NOs: 760-765. In certain embodiments, the anti-CD30L antibody or antigen binding fragment provided herein comprises or consists of a CDR-H1 comprising the amino acid sequence set forth in any one of SEQ ID NOs: 712-723, a CDR-H3 comprising the amino acid sequence set forth in any one of SEQ ID NOs: 736-743, a CDR-L2 comprising the amino acid sequence set forth in any one of SEQ ID NOs: 752-759, and a CDR-L3 comprising the amino acid sequence set forth in any one of SEQ ID NOs: 760-765. In yet other embodiments, the anti-CD30L antibody or antigen binding fragment provided herein comprises or consists of a CDR-H1 comprising the amino acid sequence set forth in any one of SEQ ID NOs: 712-723, a CDR-L1 comprising the amino acid sequence set forth in any one of SEQ ID NOs: 744-751, a CDR-L2 comprising the amino acid sequence set forth in any one of SEQ ID NOs: 752-759, and a CDR-L3 comprising the amino acid sequence set forth in any one of SEQ ID NOs: 760-765. In further embodiments, the anti-CD30L antibody or antigen binding fragment provided herein comprises or consists of a CDR-H2 comprising the amino acid sequence set forth in any one of SEQ ID NOs: 724-735, a CDR-H3 comprising the amino acid sequence set forth in any one of SEQ ID NOs: 736-743, a CDR-L1 comprising the amino acid sequence set forth in any one of SEQ ID NOs: 744-751, and a CDR-L2 comprising the amino acid sequence set forth in any one of SEQ ID NOs: 752-759. In some embodiments, the anti-CD30L antibody or antigen binding fragment provided herein comprises or consists of a CDR-H2 comprising the amino acid sequence set forth in any one of SEQ ID NOs: 724-735, a CDR-H3 comprising the amino acid sequence set forth in any one of SEQ ID NOs: 736-743, a CDR-L1 comprising the amino acid sequence set forth in any one of SEQ ID NOs: 744-751, and a CDR-L3 comprising the amino acid sequence set forth in any one of SEQ ID NOs: 760-765. In further embodiments, the anti-CD30L antibody or antigen binding fragment provided herein comprises or consists of a CDR-H2 comprising the amino acid sequence set forth in any one of SEQ ID NOs: 724-735, a CDR-H3 comprising the amino acid sequence set forth in any one of SEQ ID NOs: 736-743, a CDR-L2 comprising the amino acid sequence set forth in any one of SEQ ID NOs: 752-759, and a CDR-L3 comprising the amino acid sequence set forth in any one of SEQ ID NOs: 760-765. In some embodiments, the anti-CD30L antibody or antigen binding fragment provided herein comprises or consists of a CDR-H2 comprising the amino acid sequence set forth in any one of SEQ ID NOs: 724-735, a CDR-L1 comprising the amino acid sequence set forth in any one of SEQ ID NOs: 744-751, a CDR-L2 comprising the amino acid sequence set forth in any one of SEQ ID NOs: 752-759, and a CDR-L3 comprising the amino acid sequence set forth in any one of SEQ ID NOs: 760-765. In some embodiments, the anti-CD30L antibody or antigen binding fragment provided herein comprises or consists of a CDR-H3 comprising the amino acid sequence set forth in any one of SEQ ID NOs: 736-743, a CDR-L1 comprising the amino acid sequence set forth in any one of SEQ ID NOs: 744-751, a CDR-L2 comprising the amino acid sequence set forth in any one of SEQ ID NOs: 752-759, and a CDR-L3 comprising the amino acid sequence set forth in any one of SEQ ID NOs: 760-765. In certain embodiments, the anti-CD30L antibody or antigen binding fragment provided herein comprises or consists of a CDR-H1 comprising the amino acid sequence set forth in any one of SEQ ID NOs: 712-723, a CDR-H2 comprising the amino acid sequence set forth in any one of SEQ ID NOs: 724-735, a CDR-H3 comprising the amino acid sequence set forth in any one of SEQ ID NOs: 736-743, a CDR-L1 comprising the amino acid sequence set forth in any one of SEQ ID NOs: 744-751, and a CDR-L2 comprising the amino acid sequence set forth in any one of SEQ ID NOs: 752-759. In some embodiments, the anti-CD30L antibody or antigen binding fragment provided herein comprises or consists of a CDR-H1 comprising the amino acid sequence set forth in any one of SEQ ID NOs: 712-723, a CDR-H2 comprising the amino acid sequence set forth in any one of SEQ ID NOs: 724-735, a CDR-H3 comprising the amino acid sequence set forth in any one of SEQ ID NOs: 736-743, a CDR-L1 comprising the amino acid sequence set forth in any one of SEQ ID NOs: 744-751, and a CDR-L3 comprising the amino acid sequence set forth in any one of SEQ ID NOs: 760-765. In some embodiments, the anti-CD30L antibody or antigen binding fragment provided herein comprises or consists of a CDR-H1 comprising the amino acid sequence set forth in any one of SEQ ID NOs: 712-723, a CDR-H2 comprising the amino acid sequence set forth in any one of SEQ ID NOs: 724-735, a CDR-H3 comprising the amino acid sequence set forth in any one of SEQ ID NOs: 736-743, a CDR-L2 comprising the amino acid sequence set forth in any one of SEQ ID NOs: 752-759, and a CDR-L3 comprising the amino acid sequence set forth in any one of SEQ ID NOs: 760-765. In certain embodiments, the anti-CD30L antibody or antigen binding fragment provided herein comprises or consists of a CDR-H1 comprising the amino acid sequence set forth in any one of SEQ ID NOs: 712-723, a CDR-H2 comprising the amino acid sequence set forth in any one of SEQ ID NOs: 724-735, a CDR-L1 comprising the amino acid sequence set forth in any one of SEQ ID NOs: 744-751, a CDR-L2 comprising the amino acid sequence set forth in any one of SEQ ID NOs: 752-759, and a CDR-L3 comprising the amino acid sequence set forth in any one of SEQ ID NOs: 760-765. In certain embodiments, the anti-CD30L antibody or antigen binding fragment provided herein comprises or consists of a CDR-H1 comprising the amino acid sequence set forth in any one of SEQ ID NOs: 712-723, a CDR-H3 comprising the amino acid sequence set forth in any one of SEQ ID NOs: 736-743, a CDR-L1 comprising the amino acid sequence set forth in any one of SEQ ID NOs: 744-751, a CDR-L2 comprising the amino acid sequence set forth in any one of SEQ ID NOs: 752-759, and a CDR-L3 comprising the amino acid sequence set forth in any one of SEQ ID NOs: 760-765. In some embodiments, the anti-CD30L antibody or antigen binding fragment provided herein comprises or consists of a CDR-H2 comprising the amino acid sequence set forth in any one of SEQ ID NOs: 724-735, a CDR-H3 comprising the amino acid sequence set forth in any one of SEQ ID NOs: 736-743, a CDR-L1 comprising the amino acid sequence set forth in any one of SEQ ID NOs: 744-751, a CDR-L2 comprising the amino acid sequence set forth in any one of SEQ ID NOs: 752-759, and a CDR-L3 comprising the amino acid sequence set forth in any one of SEQ ID NOs: 760-765. In some embodiments, the anti-CD30L antibody or antigen binding fragment provided herein comprises or consists of a CDR-H1 comprising the amino acid sequence set forth in any one of SEQ ID NOs: 712-723, a CDR-H2 comprising the amino acid sequence set forth in any one of SEQ ID NOs: 724-735, a CDR-H3 comprising the amino acid sequence set forth in any one of SEQ ID NOs: 736-743, a CDR-L1 comprising the amino acid sequence set forth in any one of SEQ ID NOs: 744-751, a CDR-L2 comprising the amino acid sequence set forth in any one of SEQ ID NOs: 752-759, and a CDR-L3 comprising the amino acid sequence set forth in any one of SEQ ID NOs: 760-765. In some embodiments, the anti-CD30L antibody or antigen binding fragment provided herein comprises or consists of any one of a CDR-H1 comprising the amino acid sequence set forth in any one of SEQ ID NOs: 712-723, a CDR-H2 comprising the amino acid sequence set forth in any one of SEQ ID NOs: 724-735, a CDR-H3 comprising the amino acid sequence set forth in any one of SEQ ID NOs: 736-743, a CDR-L1 comprising the amino acid sequence set forth in any one of SEQ ID NOs: 744-751, a CDR-L2 comprising the amino acid sequence set forth in any one of SEQ ID NOs: 752-759, and a CDR-L3 comprising the amino acid sequence set forth in any one of SEQ ID NOs: 760-765. In some embodiments, the anti-CD30L antibody or antigen binding fragment provided herein comprises or consists of any two of a CDR-H1 comprising the amino acid sequence set forth in any one of SEQ ID NOs: 712-723, a CDR-H2 comprising the amino acid sequence set forth in any one of SEQ ID NOs: 724-735, a CDR-H3 comprising the amino acid sequence set forth in any one of SEQ ID NOs: 736-743, a CDR-L1 comprising the amino acid sequence set forth in any one of SEQ ID NOs: 744-751, a CDR-L2 comprising the amino acid sequence set forth in any one of SEQ ID NOs: 752-759, and a CDR-L3 comprising the amino acid sequence set forth in any one of SEQ ID NOs: 760-765, in any combination or permutation. In some embodiments, the anti-CD30L antibody or antigen binding fragment provided herein comprises or consists of any three of a CDR-H1 comprising the amino acid sequence set forth in any one of SEQ ID NOs: 712-723, a CDR-H2 comprising the amino acid sequence set forth in any one of SEQ ID NOs: 724-735, a CDR-H3 comprising the amino acid sequence set forth in any one of SEQ ID NOs: 736-743, a CDR-L1 comprising the amino acid sequence set forth in any one of SEQ ID NOs: 744-751, a CDR-L2 comprising the amino acid sequence set forth in any one of SEQ ID NOs: 752-759, and a CDR-L3 comprising the amino acid sequence set forth in any one of SEQ ID NOs: 760-765, in any combination or permutation. In some embodiments, the anti-CD30L antibody or antigen binding fragment provided herein comprises or consists of any four of a CDR-H1 comprising the amino acid sequence set forth in any one of SEQ ID NOs: 712-723, a CDR-H2 comprising the amino acid sequence set forth in any one of SEQ ID NOs: 724-735, a CDR-H3 comprising the amino acid sequence set forth in any one of SEQ ID NOs: 736-743, a CDR-L1 comprising the amino acid sequence set forth in any one of SEQ ID NOs: 744-751, a CDR-L2 comprising the amino acid sequence set forth in any one of SEQ ID NOs: 752-759, and a CDR-L3 comprising the amino acid sequence set forth in any one of SEQ ID NOs: 760-765, in any combination or permutation. In some embodiments, the anti-CD30L antibody or antigen binding fragment provided herein comprises or consists of any five of a CDR-H1 comprising the amino acid sequence set forth in any one of SEQ ID NOs: 712-723, a CDR-H2 comprising the amino acid sequence set forth in any one of SEQ ID NOs: 724-735, a CDR-H3 comprising the amino acid sequence set forth in any one of SEQ ID NOs: 736-743, a CDR-L1 comprising the amino acid sequence set forth in any one of SEQ ID NOs: 744-751, a CDR-L2 comprising the amino acid sequence set forth in any one of SEQ ID NOs: 752-759, and a CDR-L3 comprising the amino acid sequence set forth in any one of SEQ ID NOs: 760-765, in any combination or permutation. In some embodiments, the anti-CD30L antibody or antigen binding fragment provided herein comprises or consists of all six of a CDR-H1 comprising the amino acid sequence set forth in any one of SEQ ID NOs: 712-723, a CDR-H2 comprising the amino acid sequence set forth in any one of SEQ ID NOs: 724-735, a CDR-H3 comprising the amino acid sequence set forth in any one of SEQ ID NOs: 736-743, a CDR-L1 comprising the amino acid sequence set forth in any one of SEQ ID NOs: 744-751, a CDR-L2 comprising the amino acid sequence set forth in any one of SEQ ID NOs: 752-759, and a CDR-L3 comprising the amino acid sequence set forth in any one of SEQ ID NOs: 760-765. In some embodiments of the anti-CD30L provided herein including in Section 2 and this Section 4.1 (such as those of this paragraph), the CDR-H1 comprises the amino acid sequence set forth in any one of SEQ ID NOs: 105-109, 628, 635, 115-119, 629, 636, 480-484, 630, 637, 465-469, 631, 638, 470-474, 632, 639, 475-479, 633, 640, 485-489, 634, and 641. In some embodiments of the anti-CD30L provided herein including in Section 2 and this Section 4.1 (such as those of this paragraph), the CDR-H2 comprises the amino acid sequence set forth in any one of SEQ ID NOs: 145-149, 642, 649, 155-159, 643, 650, 518-522, 644, 651, 490-494, 645, 652, 495-499, 646, 653, 513-517, 647, 654, 523-527, 648, and 655. In some embodiments of the anti-CD30L provided herein including in Section 2 and this Section 4.1 (such as those of this paragraph), the CDR-H3 comprises the amino acid sequence set forth in any one of SEQ ID NOs: 185-189, 656, 663, 195-199, 657, 664, 543-547, 658, 665, 528-532, 659, 666, 533-537, 660, 667, 538-542, 661, 668, 548-552, 662, and 669. In some embodiments of the anti-CD30L provided herein including in Section 2 and this Section 4.1 (such as those of this paragraph), the CDR-L1 comprises the amino acid sequence set forth in any one of SEQ ID NOs: 305-309, 670, 677, 315-319, 671, 678, 568-572, 672, 679, 553-557, 673, 680, 558-562, 674, 681, 563-567, 675, 682, 573-577, 676, and 683. In some embodiments of the anti-CD30L provided herein including in Section 2 and this Section 4.1 (such as those of this paragraph), the CDR-L2 comprises the amino acid sequence set forth in any one of SEQ ID NOs: 345-349, 684, 691, 355-359, 685, 692, 593-597, 686, 693, 578-582, 687, 694, 583-587, 688, 695, 588-592, 689, 696, 598-602, 690, and 697. In some embodiments of the anti-CD30L provided herein including in Section 2 and this Section 4.1 (such as those of this paragraph), the CDR-L3 comprises the amino acid sequence set forth in any one of SEQ ID NOs: 385-389, 698, 705, 395-399, 699, 706, 618-622, 700, 707, 603-607, 701, 708, 608-612, 702, 709, 613-617, 703, 710, 623-627, 704, and 711. In certain embodiments of the anti-CD30L provided herein including in Section 2 and this Section 4.1 (such as those of this paragraph), the CDR-H1 comprises the amino acid sequence set forth in any one of SEQ ID NOs: 105-109, 628, 635, 115-119, 629, 636, 480-484, 630, and 637. In certain embodiments of the anti-CD30L provided herein including in Section 2 and this Section 4.1 (such as those of this paragraph), the CDR-H2 comprises the amino acid sequence set forth in any one of SEQ ID NOs: 145-149, 642, 649, 155-159, 643, 650, 518-522, 644, and 651. In certain embodiments of the anti-CD30L provided herein including in Section 2 and this Section 4.1 (such as those of this paragraph), the CDR-H3 comprises the amino acid sequence set forth in any one of SEQ ID NOs: 185-189, 656, 663, 195-199, 657, 664, 543-547, 658, and 665. In certain embodiments of the anti-CD30L provided herein including in Section 2 and this Section 4.1 (such as those of this paragraph), the CDR-L1 comprises the amino acid sequence set forth in any one of SEQ ID NOs: 305-309, 670, 677, 315-319, 671, 678, 568-572, 672, and 679. In certain embodiments of the anti-CD30L provided herein including in Section 2 and this Section 4.1 (such as those of this paragraph), the CDR-L2 comprises the amino acid sequence set forth in any one of SEQ ID NOs: 345-349, 684, 691, 355-359, 685, 692, 593-597, 686, and 693. In certain embodiments of the anti-CD30L provided herein including in Section 2 and this Section 4.1 (such as those of this paragraph), the CDR-L3 comprises the amino acid sequence set forth in any one of SEQ ID NOs: 385-389, 698, 705, 395-399, 699, 706, 618-622, 700, and 707.

Additionally, the anti-CD30L antibody provided herein, including in Section 2 and this Section (Section 4.1), can comprise specific combinations of CDRs of each anti-CD30L clone as listed in Tables 10-16. Accordingly, in one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 635; (b) a CDR-H2 comprising the amino acid sequence set forth in SEQ ID NO: 649; (c) a CDR-H3 comprising the amino acid sequence set forth in SEQ ID NO: 663; (d) a CDR-L1 comprising the amino acid sequence set forth in SEQ ID NO: 677; (e) a CDR-L2 comprising the amino acid sequence set forth SEQ ID NO: 691; and/or (f) a CDR-L3 comprising the amino acid sequence set forth SEQ ID NO: 705. In another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 107; (b) a CDR-H2 comprising the amino acid sequence set forth in SEQ ID NO: 147; (c) a CDR-H3 comprising the amino acid sequence set forth in SEQ ID NO: 187; (d) a CDR-L1 comprising the amino acid sequence set forth in SEQ ID NO: 307; (e) a CDR-L2 comprising the amino acid sequence set forth SEQ ID NO: 347; and/or (f) a CDR-L3 comprising the amino acid sequence set forth SEQ ID NO: 387. In one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 105; (b) a CDR-H2 comprising the amino acid sequence set forth in SEQ ID NO: 145; (c) a CDR-H3 comprising the amino acid sequence set forth in SEQ ID NO: 185; (d) a CDR-L1 comprising the amino acid sequence set forth in SEQ ID NO: 305; (e) a CDR-L2 comprising the amino acid sequence set forth SEQ ID NO: 345; and/or (f) a CDR-L3 comprising the amino acid sequence set forth SEQ ID NO: 385. In one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 106; (b) a CDR-H2 comprising the amino acid sequence set forth in SEQ ID NO: 146; (c) a CDR-H3 comprising the amino acid sequence set forth in SEQ ID NO: 186; (d) a CDR-L1 comprising the amino acid sequence set forth in SEQ ID NO: 306; (e) a CDR-L2 comprising the amino acid sequence set forth SEQ ID NO: 346; and/or (f) a CDR-L3 comprising the amino acid sequence set forth SEQ ID NO: 386. In one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 108; (b) a CDR-H2 comprising the amino acid sequence set forth in SEQ ID NO: 148; (c) a CDR-H3 comprising the amino acid sequence set forth in SEQ ID NO: 188; (d) a CDR-L1 comprising the amino acid sequence set forth in SEQ ID NO: 308; (e) a CDR-L2 comprising the amino acid sequence set forth SEQ ID NO: 348; and/or (f) a CDR-L3 comprising the amino acid sequence set forth SEQ ID NO: 388. In one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 109; (b) a CDR-H2 comprising the amino acid sequence set forth in SEQ ID NO: 149; (c) a CDR-H3 comprising the amino acid sequence set forth in SEQ ID NO: 189; (d) a CDR-L1 comprising the amino acid sequence set forth in SEQ ID NO: 309; (e) a CDR-L2 comprising the amino acid sequence set forth SEQ ID NO: 349; and/or (f) a CDR-L3 comprising the amino acid sequence set forth SEQ ID NO: 389. In one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 628; (b) a CDR-H2 comprising the amino acid sequence set forth in SEQ ID NO: 642; (c) a CDR-H3 comprising the amino acid sequence set forth in SEQ ID NO: 656; (d) a CDR-L1 comprising the amino acid sequence set forth in SEQ ID NO: 670; (e) a CDR-L2 comprising the amino acid sequence set forth SEQ ID NO: 684; and/or (f) a CDR-L3 comprising the amino acid sequence set forth SEQ ID NO: 698.

Similarly, in one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 636; (b) a CDR-H2 comprising the amino acid sequence set forth in SEQ ID NO: 650; (c) a CDR-H3 comprising the amino acid sequence set forth in SEQ ID NO: 664; (d) a CDR-L1 comprising the amino acid sequence set forth in SEQ ID NO: 678; (e) a CDR-L2 comprising the amino acid sequence set forth SEQ ID NO: 692; and/or (f) a CDR-L3 comprising the amino acid sequence set forth SEQ ID NO: 706. In another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 117; (b) a CDR-H2 comprising the amino acid sequence set forth in SEQ ID NO: 157; (c) a CDR-H3 comprising the amino acid sequence set forth in SEQ ID NO: 197; (d) a CDR-L1 comprising the amino acid sequence set forth in SEQ ID NO: 317; (e) a CDR-L2 comprising the amino acid sequence set forth SEQ ID NO: 357; and/or (f) a CDR-L3 comprising the amino acid sequence set forth SEQ ID NO: 397. In yet another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 115; (b) a CDR-H2 comprising the amino acid sequence set forth in SEQ ID NO: 155; (c) a CDR-H3 comprising the amino acid sequence set forth in SEQ ID NO: 195; (d) a CDR-L1 comprising the amino acid sequence set forth in SEQ ID NO: 315; (e) a CDR-L2 comprising the amino acid sequence set forth SEQ ID NO: 355; and/or (f) a CDR-L3 comprising the amino acid sequence set forth SEQ ID NO: 395. In one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 116; (b) a CDR-H2 comprising the amino acid sequence set forth in SEQ ID NO: 156; (c) a CDR-H3 comprising the amino acid sequence set forth in SEQ ID NO: 196; (d) a CDR-L1 comprising the amino acid sequence set forth in SEQ ID NO: 316; (e) a CDR-L2 comprising the amino acid sequence set forth SEQ ID NO: 356; and/or (f) a CDR-L3 comprising the amino acid sequence set forth SEQ ID NO: 396. In another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 118; (b) a CDR-H2 comprising the amino acid sequence set forth in SEQ ID NO: 158; (c) a CDR-H3 comprising the amino acid sequence set forth in SEQ ID NO: 198; (d) a CDR-L1 comprising the amino acid sequence set forth in SEQ ID NO: 318; (e) a CDR-L2 comprising the amino acid sequence set forth SEQ ID NO: 358; and/or (f) a CDR-L3 comprising the amino acid sequence set forth SEQ ID NO: 398. In yet another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 119; (b) a CDR-H2 comprising the amino acid sequence set forth in SEQ ID NO: 159; (c) a CDR-H3 comprising the amino acid sequence set forth in SEQ ID NO: 199; (d) a CDR-L1 comprising the amino acid sequence set forth in SEQ ID NO: 319; (e) a CDR-L2 comprising the amino acid sequence set forth SEQ ID NO: 359; and/or (f) a CDR-L3 comprising the amino acid sequence set forth SEQ ID NO: 399. In one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 629; (b) a CDR-H2 comprising the amino acid sequence set forth in SEQ ID NO: 643; (c) a CDR-H3 comprising the amino acid sequence set forth in SEQ ID NO: 657; (d) a CDR-L1 comprising the amino acid sequence set forth in SEQ ID NO: 671; (e) a CDR-L2 comprising the amino acid sequence set forth SEQ ID NO: 685; and/or (f) a CDR-L3 comprising the amino acid sequence set forth SEQ ID NO: 699.

Likewise, in one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 637; (b) a CDR-H2 comprising the amino acid sequence set forth in SEQ ID NO: 651; (c) a CDR-H3 comprising the amino acid sequence set forth in SEQ ID NO: 665; (d) a CDR-L1 comprising the amino acid sequence set forth in SEQ ID NO: 679; (e) a CDR-L2 comprising the amino acid sequence set forth SEQ ID NO: 693; and/or (f) a CDR-L3 comprising the amino acid sequence set forth SEQ ID NO: 707. In another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 482; (b) a CDR-H2 comprising the amino acid sequence set forth in SEQ ID NO: 520; (c) a CDR-H3 comprising the amino acid sequence set forth in SEQ ID NO: 545; (d) a CDR-L1 comprising the amino acid sequence set forth in SEQ ID NO: 570; (e) a CDR-L2 comprising the amino acid sequence set forth SEQ ID NO: 595; and/or (f) a CDR-L3 comprising the amino acid sequence set forth SEQ ID NO: 620. In yet another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 480; (b) a CDR-H2 comprising the amino acid sequence set forth in SEQ ID NO: 518; (c) a CDR-H3 comprising the amino acid sequence set forth in SEQ ID NO: 543; (d) a CDR-L1 comprising the amino acid sequence set forth in SEQ ID NO: 568; (e) a CDR-L2 comprising the amino acid sequence set forth SEQ ID NO: 593; and/or (f) a CDR-L3 comprising the amino acid sequence set forth SEQ ID NO: 618. In one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 481; (b) a CDR-H2 comprising the amino acid sequence set forth in SEQ ID NO: 519; (c) a CDR-H3 comprising the amino acid sequence set forth in SEQ ID NO: 544; (d) a CDR-L1 comprising the amino acid sequence set forth in SEQ ID NO: 569; (e) a CDR-L2 comprising the amino acid sequence set forth SEQ ID NO: 594; and/or (f) a CDR-L3 comprising the amino acid sequence set forth SEQ ID NO: 619. In another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 483; (b) a CDR-H2 comprising the amino acid sequence set forth in SEQ ID NO: 521; (c) a CDR-H3 comprising the amino acid sequence set forth in SEQ ID NO: 546; (d) a CDR-L1 comprising the amino acid sequence set forth in SEQ ID NO: 571; (e) a CDR-L2 comprising the amino acid sequence set forth SEQ ID NO: 596; and/or (f) a CDR-L3 comprising the amino acid sequence set forth SEQ ID NO: 621. In yet another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 484; (b) a CDR-H2 comprising the amino acid sequence set forth in SEQ ID NO: 522; (c) a CDR-H3 comprising the amino acid sequence set forth in SEQ ID NO: 547; (d) a CDR-L1 comprising the amino acid sequence set forth in SEQ ID NO: 572; (e) a CDR-L2 comprising the amino acid sequence set forth SEQ ID NO: 597; and/or (f) a CDR-L3 comprising the amino acid sequence set forth SEQ ID NO: 622. In one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 630; (b) a CDR-H2 comprising the amino acid sequence set forth in SEQ ID NO: 644; (c) a CDR-H3 comprising the amino acid sequence set forth in SEQ ID NO: 658; (d) a CDR-L1 comprising the amino acid sequence set forth in SEQ ID NO: 672; (e) a CDR-L2 comprising the amino acid sequence set forth SEQ ID NO: 686; and/or (f) a CDR-L3 comprising the amino acid sequence set forth SEQ ID NO: 700.

Additionally, in one embodiment of the anti-CD30L antibody or antigen binding fragment provided herein, including in Section 2 and this Section (Section 4.1), the antibody or antigen binding fragment thereof comprises: (i)(a) a CDR-H1 comprising the amino acid sequence set forth in SEQ ID NO: 638; (b) a CDR-H2 comprising the amino acid sequence set forth in SEQ ID NO: 652; (c) a CDR-H3 comprising the amino acid sequence set forth in SEQ ID NO: 666; (d) a CDR-L1 comprising the amino acid sequence set forth in SEQ ID NO: 680; (e) a CDR-L2 comprising the amino acid sequence set forth SEQ ID NO: 694; and/or (f) a CDR-L3 comprising the amino acid sequence set forth SEQ ID NO: 708. In another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 467; (b) a CDR-H2 comprising the amino acid sequence set forth in SEQ ID NO: 492; (c) a CDR-H3 comprising the amino acid sequence set forth in SEQ ID NO: 530; (d) a CDR-L1 comprising the amino acid sequence set forth in SEQ ID NO: 555; (e) a CDR-L2 comprising the amino acid sequence set forth SEQ ID NO: 580; and/or (f) a CDR-L3 comprising the amino acid sequence set forth SEQ ID NO: 605. In yet another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 465; (b) a CDR-H2 comprising the amino acid sequence set forth in SEQ ID NO: 490; (c) a CDR-H3 comprising the amino acid sequence set forth in SEQ ID NO: 528; (d) a CDR-L1 comprising the amino acid sequence set forth in SEQ ID NO: 553; (e) a CDR-L2 comprising the amino acid sequence set forth SEQ ID NO: 578; and/or (f) a CDR-L3 comprising the amino acid sequence set forth SEQ ID NO: 603. In one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 466; (b) a CDR-H2 comprising the amino acid sequence set forth in SEQ ID NO: 491; (c) a CDR-H3 comprising the amino acid sequence set forth in SEQ ID NO: 529; (d) a CDR-L1 comprising the amino acid sequence set forth in SEQ ID NO: 554; (e) a CDR-L2 comprising the amino acid sequence set forth SEQ ID NO: 579; and/or (f) a CDR-L3 comprising the amino acid sequence set forth SEQ ID NO: 604. In another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 468; (b) a CDR-H2 comprising the amino acid sequence set forth in SEQ ID NO: 493; (c) a CDR-H3 comprising the amino acid sequence set forth in SEQ ID NO: 531; (d) a CDR-L1 comprising the amino acid sequence set forth in SEQ ID NO: 556; (e) a CDR-L2 comprising the amino acid sequence set forth SEQ ID NO: 581; and/or (f) a CDR-L3 comprising the amino acid sequence set forth SEQ ID NO: 606. In yet another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 469; (b) a CDR-H2 comprising the amino acid sequence set forth in SEQ ID NO: 494; (c) a CDR-H3 comprising the amino acid sequence set forth in SEQ ID NO: 532; (d) a CDR-L1 comprising the amino acid sequence set forth in SEQ ID NO: 557; (e) a CDR-L2 comprising the amino acid sequence set forth SEQ ID NO: 582; and/or (f) a CDR-L3 comprising the amino acid sequence set forth SEQ ID NO: 607. In one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 631; (b) a CDR-H2 comprising the amino acid sequence set forth in SEQ ID NO: 645; (c) a CDR-H3 comprising the amino acid sequence set forth in SEQ ID NO: 659; (d) a CDR-L1 comprising the amino acid sequence set forth in SEQ ID NO: 673; (e) a CDR-L2 comprising the amino acid sequence set forth SEQ ID NO: 687; and/or (f) a CDR-L3 comprising the amino acid sequence set forth SEQ ID NO: 701.

Similarly, in one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 639; (b) a CDR-H2 comprising the amino acid sequence set forth in SEQ ID NO: 653; (c) a CDR-H3 comprising the amino acid sequence set forth in SEQ ID NO: 667; (d) a CDR-L1 comprising the amino acid sequence set forth in SEQ ID NO: 681; (e) a CDR-L2 comprising the amino acid sequence set forth SEQ ID NO: 695; and/or (f) a CDR-L3 comprising the amino acid sequence set forth SEQ ID NO: 709. In another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 472; (b) a CDR-H2 comprising the amino acid sequence set forth in SEQ ID NO: 497; (c) a CDR-H3 comprising the amino acid sequence set forth in SEQ ID NO: 535; (d) a CDR-L1 comprising the amino acid sequence set forth in SEQ ID NO: 560; (e) a CDR-L2 comprising the amino acid sequence set forth SEQ ID NO: 585; and/or (f) a CDR-L3 comprising the amino acid sequence set forth SEQ ID NO: 610. In yet another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 470; (b) a CDR-H2 comprising the amino acid sequence set forth in SEQ ID NO: 495; (c) a CDR-H3 comprising the amino acid sequence set forth in SEQ ID NO: 533; (d) a CDR-L1 comprising the amino acid sequence set forth in SEQ ID NO: 558; (e) a CDR-L2 comprising the amino acid sequence set forth SEQ ID NO: 583; and/or (f) a CDR-L3 comprising the amino acid sequence set forth SEQ ID NO: 608. In one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 471; (b) a CDR-H2 comprising the amino acid sequence set forth in SEQ ID NO: 496; (c) a CDR-H3 comprising the amino acid sequence set forth in SEQ ID NO: 534; (d) a CDR-L1 comprising the amino acid sequence set forth in SEQ ID NO: 559; (e) a CDR-L2 comprising the amino acid sequence set forth SEQ ID NO: 584; and/or (f) a CDR-L3 comprising the amino acid sequence set forth SEQ ID NO: 609. In another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 473; (b) a CDR-H2 comprising the amino acid sequence set forth in SEQ ID NO: 498; (c) a CDR-H3 comprising the amino acid sequence set forth in SEQ ID NO: 536; (d) a CDR-L1 comprising the amino acid sequence set forth in SEQ ID NO: 561; (e) a CDR-L2 comprising the amino acid sequence set forth SEQ ID NO: 586; and/or (f) a CDR-L3 comprising the amino acid sequence set forth SEQ ID NO: 611. In yet another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 474; (b) a CDR-H2 comprising the amino acid sequence set forth in SEQ ID NO: 499; (c) a CDR-H3 comprising the amino acid sequence set forth in SEQ ID NO: 537; (d) a CDR-L1 comprising the amino acid sequence set forth in SEQ ID NO: 562; (e) a CDR-L2 comprising the amino acid sequence set forth SEQ ID NO: 587; and/or (f) a CDR-L3 comprising the amino acid sequence set forth SEQ ID NO: 612. In one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 632; (b) a CDR-H2 comprising the amino acid sequence set forth in SEQ ID NO: 646; (c) a CDR-H3 comprising the amino acid sequence set forth in SEQ ID NO: 660; (d) a CDR-L1 comprising the amino acid sequence set forth in SEQ ID NO: 674; (e) a CDR-L2 comprising the amino acid sequence set forth SEQ ID NO: 688; and/or (f) a CDR-L3 comprising the amino acid sequence set forth SEQ ID NO: 702.

Likewise, in one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 640; (b) a CDR-H2 comprising the amino acid sequence set forth in SEQ ID NO: 654; (c) a CDR-H3 comprising the amino acid sequence set forth in SEQ ID NO: 668; (d) a CDR-L1 comprising the amino acid sequence set forth in SEQ ID NO: 682; (e) a CDR-L2 comprising the amino acid sequence set forth SEQ ID NO: 696; and/or (f) a CDR-L3 comprising the amino acid sequence set forth SEQ ID NO: 710. In another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 477; (b) a CDR-H2 comprising the amino acid sequence set forth in SEQ ID NO: 515; (c) a CDR-H3 comprising the amino acid sequence set forth in SEQ ID NO: 540; (d) a CDR-L1 comprising the amino acid sequence set forth in SEQ ID NO: 565; (e) a CDR-L2 comprising the amino acid sequence set forth SEQ ID NO: 590; and/or (f) a CDR-L3 comprising the amino acid sequence set forth SEQ ID NO: 615. In yet another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 475; (b) a CDR-H2 comprising the amino acid sequence set forth in SEQ ID NO: 513; (c) a CDR-H3 comprising the amino acid sequence set forth in SEQ ID NO: 538; (d) a CDR-L1 comprising the amino acid sequence set forth in SEQ ID NO: 563; (e) a CDR-L2 comprising the amino acid sequence set forth SEQ ID NO: 588; and/or (f) a CDR-L3 comprising the amino acid sequence set forth SEQ ID NO: 613. In one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 476; (b) a CDR-H2 comprising the amino acid sequence set forth in SEQ ID NO: 514; (c) a CDR-H3 comprising the amino acid sequence set forth in SEQ ID NO: 539; (d) a CDR-L1 comprising the amino acid sequence set forth in SEQ ID NO: 564; (e) a CDR-L2 comprising the amino acid sequence set forth SEQ ID NO: 589; and/or (f) a CDR-L3 comprising the amino acid sequence set forth SEQ ID NO: 614. In another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 478; (b) a CDR-H2 comprising the amino acid sequence set forth in SEQ ID NO: 516; (c) a CDR-H3 comprising the amino acid sequence set forth in SEQ ID NO: 541; (d) a CDR-L1 comprising the amino acid sequence set forth in SEQ ID NO: 566; (e) a CDR-L2 comprising the amino acid sequence set forth SEQ ID NO: 591; and/or (f) a CDR-L3 comprising the amino acid sequence set forth SEQ ID NO: 616. In yet another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 479; (b) a CDR-H2 comprising the amino acid sequence set forth in SEQ ID NO: 517; (c) a CDR-H3 comprising the amino acid sequence set forth in SEQ ID NO: 542; (d) a CDR-L1 comprising the amino acid sequence set forth in SEQ ID NO: 567; (e) a CDR-L2 comprising the amino acid sequence set forth SEQ ID NO: 592; and/or (f) a CDR-L3 comprising the amino acid sequence set forth SEQ ID NO: 617. In one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 633; (b) a CDR-H2 comprising the amino acid sequence set forth in SEQ ID NO: 647; (c) a CDR-H3 comprising the amino acid sequence set forth in SEQ ID NO: 661; (d) a CDR-L1 comprising the amino acid sequence set forth in SEQ ID NO: 675; (e) a CDR-L2 comprising the amino acid sequence set forth SEQ ID NO: 689; and/or (f) a CDR-L3 comprising the amino acid sequence set forth SEQ ID NO: 703.

Additionally, in one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 641; (b) a CDR-H2 comprising the amino acid sequence set forth in SEQ ID NO: 655; (c) a CDR-H3 comprising the amino acid sequence set forth in SEQ ID NO: 669; (d) a CDR-L1 comprising the amino acid sequence set forth in SEQ ID NO: 683; (e) a CDR-L2 comprising the amino acid sequence set forth SEQ ID NO: 697; and/or (f) a CDR-L3 comprising the amino acid sequence set forth SEQ ID NO: 711. In another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 487; (b) a CDR-H2 comprising the amino acid sequence set forth in SEQ ID NO: 525; (c) a CDR-H3 comprising the amino acid sequence set forth in SEQ ID NO: 550; (d) a CDR-L1 comprising the amino acid sequence set forth in SEQ ID NO: 575; (e) a CDR-L2 comprising the amino acid sequence set forth SEQ ID NO: 600; and/or (f) a CDR-L3 comprising the amino acid sequence set forth SEQ ID NO: 625. In yet another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 485; (b) a CDR-H2 comprising the amino acid sequence set forth in SEQ ID NO: 523; (c) a CDR-H3 comprising the amino acid sequence set forth in SEQ ID NO: 548; (d) a CDR-L1 comprising the amino acid sequence set forth in SEQ ID NO: 573; (e) a CDR-L2 comprising the amino acid sequence set forth SEQ ID NO: 598; and/or (f) a CDR-L3 comprising the amino acid sequence set forth SEQ ID NO: 623. In one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 486; (b) a CDR-H2 comprising the amino acid sequence set forth in SEQ ID NO: 524; (c) a CDR-H3 comprising the amino acid sequence set forth in SEQ ID NO: 549; (d) a CDR-L1 comprising the amino acid sequence set forth in SEQ ID NO: 574; (e) a CDR-L2 comprising the amino acid sequence set forth SEQ ID NO: 599; and/or (f) a CDR-L3 comprising the amino acid sequence set forth SEQ ID NO: 624. In another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 488; (b) a CDR-H2 comprising the amino acid sequence set forth in SEQ ID NO: 526; (c) a CDR-H3 comprising the amino acid sequence set forth in SEQ ID NO: 551; (d) a CDR-L1 comprising the amino acid sequence set forth in SEQ ID NO: 576; (e) a CDR-L2 comprising the amino acid sequence set forth SEQ ID NO: 601; and/or (f) a CDR-L3 comprising the amino acid sequence set forth SEQ ID NO: 626. In yet another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 489; (b) a CDR-H2 comprising the amino acid sequence set forth in SEQ ID NO: 527; (c) a CDR-H3 comprising the amino acid sequence set forth in SEQ ID NO: 552; (d) a CDR-L1 comprising the amino acid sequence set forth in SEQ ID NO: 577; (e) a CDR-L2 comprising the amino acid sequence set forth SEQ ID NO: 602; and/or (f) a CDR-L3 comprising the amino acid sequence set forth SEQ ID NO: 627. In one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 634; (b) a CDR-H2 comprising the amino acid sequence set forth in SEQ ID NO: 648; (c) a CDR-H3 comprising the amino acid sequence set forth in SEQ ID NO: 662; (d) a CDR-L1 comprising the amino acid sequence set forth in SEQ ID NO: 676; (e) a CDR-L2 comprising the amino acid sequence set forth SEQ ID NO: 690; and/or (f) a CDR-L3 comprising the amino acid sequence set forth SEQ ID NO: 704.

The anti-CD30L antibodies can have various CDR sequences as listed in Tables 10-16. Accordingly, in one embodiment of the anti-CD30L antibody or antigen binding fragment provided herein, including in Section 2 and this Section (Section 4.1), the antibody or antigen binding fragment thereof comprises: (a) a CDR-H1 comprising any CDR-H1 sequence listed in Table 10; (b) a CDR-H2 comprising any CDR-H2 sequence listed in Table 10; (c) a CDR-H3 comprising any CDR-H3 sequence listed in Table 10; (d) a CDR-L1 comprising any CDR-L1 sequence listed in Table 10; (e) a CDR-L2 comprising any CDR-L2 sequence listed in Table 10; and/or (f) a CDR-L3 comprising any CDR-L3 sequence listed in Table 10.

In another embodiment of the anti-CD30L antibody or antigen binding fragment provided herein, including in Section 2 and this Section (Section 4.1), the antibody or antigen binding fragment thereof comprises: (a) a CDR-H1 comprising any CDR-H1 sequence listed in Table 11; (b) a CDR-H2 comprising any CDR-H2 sequence listed in Table 11; (c) a CDR-H3 comprising any CDR-H3 sequence listed in Table 11; (d) a CDR-L1 comprising any CDR-L1 sequence listed in Table 11; (e) a CDR-L2 comprising any CDR-L2 sequence listed in Table 11; and/or (f) a CDR-L3 comprising any CDR-L3 sequence listed in Table 11.

In a further embodiment of the anti-CD30L antibody or antigen binding fragment provided herein, including in Section 2 and this Section (Section 4.1), the antibody or antigen binding fragment thereof comprises: (a) a CDR-H1 comprising any CDR-H1 sequence listed in Table 12; (b) a CDR-H2 comprising any CDR-H2 sequence listed in Table 12; (c) a CDR-H3 comprising any CDR-H3 sequence listed in Table 12; (d) a CDR-L1 comprising any CDR-L1 sequence listed in Table 12; (e) a CDR-L2 comprising any CDR-L2 sequence listed in Table 12; and/or (f) a CDR-L3 comprising any CDR-L3 sequence listed in Table 12.

In yet another embodiment of the anti-CD30L antibody or antigen binding fragment provided herein, including in Section 2 and this Section (Section 4.1), the antibody or antigen binding fragment thereof comprises: (a) a CDR-H1 comprising any CDR-H1 sequence listed in Table 13; (b) a CDR-H2 comprising any CDR-H2 sequence listed in Table 13; (c) a CDR-H3 comprising any CDR-H3 sequence listed in Table 13; (d) a CDR-L1 comprising any CDR-L1 sequence listed in Table 13; (e) a CDR-L2 comprising any CDR-L2 sequence listed in Table 13; and/or (f) a CDR-L3 comprising any CDR-L3 sequence listed in Table 13.

In one embodiment of the anti-CD30L antibody or antigen binding fragment provided herein, including in Section 2 and this Section (Section 4.1), the antibody or antigen binding fragment thereof comprises: (a) a CDR-H1 comprising any CDR-H1 sequence listed in Table 14; (b) a CDR-H2 comprising any CDR-H2 sequence listed in Table 14; (c) a CDR-H3 comprising any CDR-H3 sequence listed in Table 14; (d) a CDR-L1 comprising any CDR-L1 sequence listed in Table 14; (e) a CDR-L2 comprising any CDR-L2 sequence listed in Table 14; and/or (f) a CDR-L3 comprising any CDR-L3 sequence listed in Table 14.

In another embodiment of the anti-CD30L antibody or antigen binding fragment provided herein, including in Section 2 and this Section (Section 4.1), the antibody or antigen binding fragment thereof comprises: (a) a CDR-H1 comprising any CDR-H1 sequence listed in Table 15; (b) a CDR-H2 comprising any CDR-H2 sequence listed in Table 15; (c) a CDR-H3 comprising any CDR-H3 sequence listed in Table 15; (d) a CDR-L1 comprising any CDR-L1 sequence listed in Table 15; (e) a CDR-L2 comprising any CDR-L2 sequence listed in Table 15; and/or (f) a CDR-L3 comprising any CDR-L3 sequence listed in Table 15.

In a further embodiment of the anti-CD30L antibody or antigen binding fragment provided herein, including in Section 2 and this Section (Section 4.1), the antibody or antigen binding fragment thereof comprises: (a) a CDR-H1 comprising any CDR-H1 sequence listed in Table 16; (b) a CDR-H2 comprising any CDR-H2 sequence listed in Table 16; (c) a CDR-H3 comprising any CDR-H3 sequence listed in Table 16; (d) a CDR-L1 comprising any CDR-L1 sequence listed in Table 16; (e) a CDR-L2 comprising any CDR-L2 sequence listed in Table 16; and/or (f) a CDR-L3 comprising any CDR-L3 sequence listed in Table 16.

In a further embodiment of the anti-CD30L antibody or antigen binding fragment provided herein, including in Section 2 and this Section (Section 4.1), the antibody or antigen binding fragment thereof comprises: (a) a CDR-H1 comprising any CDR-H1 sequence listed in Table 27; (b) a CDR-H2 comprising any CDR-H2 sequence listed in Table 27; (c) a CDR-H3 comprising any CDR-H3 sequence listed in Table 27; (d) a CDR-L1 comprising any CDR-L1 sequence listed in Table 27; (e) a CDR-L2 comprising any CDR-L2 sequence listed in Table 17; and/or (f) a CDR-L3 comprising any CDR-L3 sequence listed in Table 27.

In yet another embodiment of the anti-CD30L antibody or antigen binding fragment provided herein, including in Section 2 and this Section (Section 4.1), the antibody or antigen binding fragment thereof comprises: (a) a CDR-H1 comprising any CDR-H1 sequence listed in Tables 10-16; (b) a CDR-H2 comprising any CDR-H2 sequence listed in Tables 10-16; (c) a CDR-H3 comprising any CDR-H3 sequence listed in Tables 10-16; (d) a CDR-L1 comprising any CDR-L1 sequence listed in Tables 10-16; (e) a CDR-L2 comprising any CDR-L2 sequence listed in Tables 10-16; and/or (f) a CDR-L3 comprising any CDR-L3 sequence listed in Tables 10-16.

In some embodiments of the anti-CD30L antibody or antigen binding fragment provided herein, including in Section 2 and this Section (Section 4.1), the antibody or antigen binding fragment thereof comprises the CDR-H1, CDR-H2, CDR-H3, CDR-L1, CDR-L2, and CDR-L3 of the corresponding CDR-H1, CDR-H2, CDR-H3, CDR-L1, CDR-L2, and CDR-L3 of any anti-CD30L of the same clone number listed in Tables 10-16 and 27. In some embodiments of the anti-CD30L antibody or antigen binding fragment provided herein, including in Section 2 and this Section (Section 4.1), the antibody or antigen binding fragment thereof comprises the CDR-H1, CDR-H2, CDR-H3, CDR-L1, CDR-L2, and CDR-L3 of the corresponding CDR-H1, CDR-H2, CDR-H3, CDR-L1, CDR-L2, and CDR-L3 of any anti-CD30L of the same clone number listed in Table 10. In some embodiments of the anti-CD30L antibody or antigen binding fragment provided herein, including in Section 2 and this Section (Section 4.1), the antibody or antigen binding fragment thereof comprises the CDR-H1, CDR-H2, CDR-H3, CDR-L1, CDR-L2, and CDR-L3 of the corresponding CDR-H1, CDR-H2, CDR-H3, CDR-L1, CDR-L2, and CDR-L3 of any anti-CD30L of the same clone number listed in Table 11. In some embodiments of the anti-CD30L antibody or antigen binding fragment provided herein, including in Section 2 and this Section (Section 4.1), the antibody or antigen binding fragment thereof comprises the CDR-H1, CDR-H2, CDR-H3, CDR-L1, CDR-L2, and CDR-L3 of the corresponding CDR-H1, CDR-H2, CDR-H3, CDR-L1, CDR-L2, and CDR-L3 of any anti-CD30L of the same clone number listed in Table 12. In some embodiments of the anti-CD30L antibody or antigen binding fragment provided herein, including in Section 2 and this Section (Section 4.1), the antibody or antigen binding fragment thereof comprises the CDR-H1, CDR-H2, CDR-H3, CDR-L1, CDR-L2, and CDR-L3 of the corresponding CDR-H1, CDR-H2, CDR-H3, CDR-L1, CDR-L2, and CDR-L3 of any anti-CD30L of the same clone number listed in Table 13. In some embodiments of the anti-CD30L antibody or antigen binding fragment provided herein, including in Section 2 and this Section (Section 4.1), the antibody or antigen binding fragment thereof comprises the CDR-H1, CDR-H2, CDR-H3, CDR-L1, CDR-L2, and CDR-L3 of the corresponding CDR-H1, CDR-H2, CDR-H3, CDR-L1, CDR-L2, and CDR-L3 of any anti-CD30L of the same clone number listed in Table 14. In some embodiments of the anti-CD30L antibody or antigen binding fragment provided herein, including in Section 2 and this Section (Section 4.1), the antibody or antigen binding fragment thereof comprises the CDR-H1, CDR-H2, CDR-H3, CDR-L1, CDR-L2, and CDR-L3 of the corresponding CDR-H1, CDR-H2, CDR-H3, CDR-L1, CDR-L2, and CDR-L3 of any anti-CD30L of the same clone number listed in Table 15. In some embodiments of the anti-CD30L antibody or antigen binding fragment provided herein, including in Section 2 and this Section (Section 4.1), the antibody or antigen binding fragment thereof comprises the CDR-H1, CDR-H2, CDR-H3, CDR-L1, CDR-L2, and CDR-L3 of the corresponding CDR-H1, CDR-H2, CDR-H3, CDR-L1, CDR-L2, and CDR-L3 of any anti-CD30L of the same clone number listed in Table 16. In some embodiments of the anti-CD30L antibody or antigen binding fragment provided herein, including in Section 2 and this Section (Section 4.1), the antibody or antigen binding fragment thereof comprises the CDR-H1, CDR-H2, CDR-H3, CDR-L1, CDR-L2, and CDR-L3 of the corresponding CDR-H1, CDR-H2, CDR-H3, CDR-L1, CDR-L2, and CDR-L3 of any anti-CD30L of the same clone number listed in Table 27.

In some embodiments of the anti-CD30L antibody or antigen binding fragment provided herein, including in Section 2 and this Section (Section 4.1), the antibody or antigen binding fragment thereof comprises the VH and VL of the corresponding VH and VL of any anti-CD30L of the same clone number in Tables 10-16 and 27. In some embodiments of the anti-CD30L antibody or antigen binding fragment provided herein, including in Section 2 and this Section (Section 4.1), the antibody or antigen binding fragment thereof comprises the VH and VL of the corresponding VH and VL of any anti-CD30L of the same clone number listed in Table 10. In some embodiments of the anti-CD30L antibody or antigen binding fragment provided herein, including in Section 2 and this Section (Section 4.1), the antibody or antigen binding fragment thereof comprises the VH and VL of the corresponding VH and VL of any anti-CD30L of the same clone number listed in Table 11. In some embodiments of the anti-CD30L antibody or antigen binding fragment provided herein, including in Section 2 and this Section (Section 4.1), the antibody or antigen binding fragment thereof comprises the VH and VL of the corresponding VH and VL of any anti-CD30L of the same clone number listed in Table 12. In some embodiments of the anti-CD30L antibody or antigen binding fragment provided herein, including in Section 2 and this Section (Section 4.1), the antibody or antigen binding fragment thereof comprises the VH and VL of the corresponding VH and VL of any anti-CD30L of the same clone number listed in Table 13. In some embodiments of the anti-CD30L antibody or antigen binding fragment provided herein, including in Section 2 and this Section (Section 4.1), the antibody or antigen binding fragment thereof comprises the VH and VL of the corresponding VH and VL of any anti-CD30L of the same clone number listed in Table 14. In some embodiments of the anti-CD30L antibody or antigen binding fragment provided herein, including in Section 2 and this Section (Section 4.1), the antibody or antigen binding fragment thereof comprises the VH and VL of the corresponding VH and VL of any anti-CD30L of the same clone number listed in Table 15. In some embodiments of the anti-CD30L antibody or antigen binding fragment provided herein, including in Section 2 and this Section (Section 4.1), the antibody or antigen binding fragment thereof comprises the VH and VL of the corresponding VH and VL of any anti-CD30L of the same clone number listed in Table 16. In some embodiments of the anti-CD30L antibody or antigen binding fragment provided herein, including in Section 2 and this Section (Section 4.1), the antibody or antigen binding fragment thereof comprises the VH and VL of the corresponding VH and VL of any anti-CD30L of the same clone number listed in Table 27.

The disclosure also provides that the anti-CD30L antibodies provided herein can comprise various combinations of immunoglobulin heavy chain variable region (VH) and immunoglobulin light chain variable region (VL). In one embodiment of the anti-CD30L antibody or antigen binding fragment provided herein, including in Section 2 and this Section (Section 4.1), the antibody or antigen binding fragment thereof comprises: (a) an immunoglobulin variable region heavy chain (VH) comprising an amino acid sequence having at least about 90, 95, 97, 98, 99, or 100% sequence identity to any one of SEQ ID NOs: 1, 2, 5, 6, 9, 10, 13, 14, 17, 19, 21, 23, 25, 27, 29, and 31; and/or (b) an immunoglobulin variable region light chain (VL) comprising an amino acid sequence having at least about 90, 95, 97, 98, 99, or 100% sequence identity to any one of SEQ ID NOs: 3, 4, 7, 8, 11, 12, 15, 16, 18, 20, 22, 24, 26, 28, and 30. In one embodiment of the anti-CD30L antibody or antigen binding fragment provided herein, including in Section 2 and this Section (Section 4.1), the antibody or antigen binding fragment thereof comprises: (a) a VH comprising an amino acid sequence having at least about 90, 95, 97, 98, 99, or 100% sequence identity to SEQ ID NO: 1; and/or (b) a VL comprising an amino acid sequence having at least about 90, 95, 97, 98, 99, or 100% sequence identity to SEQ ID NO: 3. In one embodiment of the anti-CD30L antibody or antigen binding fragment provided herein, including in Section 2 and this Section (Section 4.1), the antibody or antigen binding fragment thereof comprises: (a) a VH comprising an amino acid sequence having at least about 90, 95, 97, 98, 99, or 100% sequence identity to SEQ ID NO: 2; and/or (b) a VL comprising an amino acid sequence having at least about 90, 95, 97, 98, 99, or 100% sequence identity to SEQ ID NO: 4. In one embodiment of the anti-CD30L antibody or antigen binding fragment provided herein, including in Section 2 and this Section (Section 4.1), the antibody or antigen binding fragment thereof comprises: (a) a VH comprising an amino acid sequence having at least about 90, 95, 97, 98, 99, or 100% sequence identity to SEQ ID NO: 5; and/or (b) a VL comprising an amino acid sequence having at least about 90, 95, 97, 98, 99, or 100% sequence identity to SEQ ID NO: 7. In one embodiment of the anti-CD30L antibody or antigen binding fragment provided herein, including in Section 2 and this Section (Section 4.1), the antibody or antigen binding fragment thereof comprises: (a) a VH comprising an amino acid sequence having at least about 90, 95, 97, 98, 99, or 100% sequence identity to SEQ ID NO: 6; and/or (b) a VL comprising an amino acid sequence having at least about 90, 95, 97, 98, 99, or 100% sequence identity to SEQ ID NO: 8. In one embodiment of the anti-CD30L antibody or antigen binding fragment provided herein, including in Section 2 and this Section (Section 4.1), the antibody or antigen binding fragment thereof comprises: (a) a VH comprising an amino acid sequence having at least about 90, 95, 97, 98, 99, or 100% sequence identity to SEQ ID NO: 9; and/or (b) a VL comprising an amino acid sequence having at least about 90, 95, 97, 98, 99, or 100% sequence identity to SEQ ID NO: 11. In one embodiment of the anti-CD30L antibody or antigen binding fragment provided herein, including in Section 2 and this Section (Section 4.1), the antibody or antigen binding fragment thereof comprises: (a) a VH comprising an amino acid sequence having at least about 90, 95, 97, 98, 99, or 100% sequence identity to SEQ ID NO: 10; and/or (b) a VL comprising an amino acid sequence having at least about 90, 95, 97, 98, 99, or 100% sequence identity to SEQ ID NO: 12. In one embodiment of the anti-CD30L antibody or antigen binding fragment provided herein, including in Section 2 and this Section (Section 4.1), the antibody or antigen binding fragment thereof comprises: (a) a VH comprising an amino acid sequence having at least about 90, 95, 97, 98, 99, or 100% sequence identity to SEQ ID NO: 13; and/or (b) a VL comprising an amino acid sequence having at least about 90, 95, 97, 98, 99, or 100% sequence identity to SEQ ID NO: 15. In one embodiment of the anti-CD30L antibody or antigen binding fragment provided herein, including in Section 2 and this Section (Section 4.1), the antibody or antigen binding fragment thereof comprises: (a) a VH comprising an amino acid sequence having at least about 90, 95, 97, 98, 99, or 100% sequence identity to SEQ ID NO: 14; and/or (b) a VL comprising an amino acid sequence having at least about 90, 95, 97, 98, 99, or 100% sequence identity to SEQ ID NO: 16. In one embodiment of the anti-CD30L antibody or antigen binding fragment provided herein, including in Section 2 and this Section (Section 4.1), the antibody or antigen binding fragment thereof comprises: (a) a VH comprising an amino acid sequence having at least about 90, 95, 97, 98, 99, or 100% sequence identity to SEQ ID NO: 23; and/or (b) a VL comprising an amino acid sequence having at least about 90, 95, 97, 98, 99, or 100% sequence identity to SEQ ID NO: 24. In one embodiment of the anti-CD30L antibody or antigen binding fragment provided herein, including in Section 2 and this Section (Section 4.1), the antibody or antigen binding fragment thereof comprises: (a) a VH comprising an amino acid sequence having at least about 90, 95, 97, 98, 99, or 100% sequence identity to SEQ ID NO: 25; and/or (b) a VL comprising an amino acid sequence having at least about 90, 95, 97, 98, 99, or 100% sequence identity to SEQ ID NO: 26. In one embodiment of the anti-CD30L antibody or antigen binding fragment provided herein, including in Section 2 and this Section (Section 4.1), the antibody or antigen binding fragment thereof comprises: (a) a VH comprising an amino acid sequence having at least about 90, 95, 97, 98, 99, or 100% sequence identity to SEQ ID NO: 27; and/or (b) a VL comprising an amino acid sequence having at least about 90, 95, 97, 98, 99, or 100% sequence identity to SEQ ID NO: 28. In one embodiment of the anti-CD30L antibody or antigen binding fragment provided herein, including in Section 2 and this Section (Section 4.1), the antibody or antigen binding fragment thereof comprises: (a) a VH comprising an amino acid sequence having at least about 90, 95, 97, 98, 99, or 100% sequence identity to SEQ ID NO: 29; and/or (b) a VL comprising an amino acid sequence having at least about 90, 95, 97, 98, 99, or 100% sequence identity to SEQ ID NO: 30. In one embodiment of the anti-CD30L antibody or antigen binding fragment provided herein, including in Section 2 and this Section (Section 4.1), the antibody or antigen binding fragment thereof comprises: (a) a VH comprising an amino acid sequence having at least about 90, 95, 97, 98, 99, or 100% sequence identity to SEQ ID NO: 31; and/or (b) a VL comprising an amino acid sequence having at least about 90, 95, 97, 98, 99, or 100% sequence identity to SEQ ID NO: 32.

Additionally, in one embodiment of the anti-CD30L antibody or antigen binding fragment provided herein, including in Section 2 and this Section (Section 4.1), the antibody or antigen binding fragment thereof comprises: (a) an immunoglobulin variable region heavy chain (VH) comprising an amino acid sequence having at least about 80, 81, 82, 83, 84, 85, 86, 87, 88, 89, 90, 91, 92, 93, 94, 95, 96, 97, 98, 99% sequence identity to any one of SEQ ID NOs: 1, 2, 5, 6, 9, 10, 13, 14, 17, 19, 21, 23, 25, 27, 29, and 31; and/or (b) an immunoglobulin variable region light chain (VL) comprising an amino acid sequence having at least about 80, 81, 82, 83, 84, 85, 86, 87, 88, 89, 90, 91, 92, 93, 94, 95, 96, 97, 98, 99% sequence identity to any one of SEQ ID NOs: 3, 4, 7, 8, 11, 12, 15, 16, 18, 20, 22, 24, 26, 28, and 30. In one embodiment of the anti-CD30L antibody or antigen binding fragment provided herein, including in Section 2 and this Section (Section 4.1), the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 1; and/or (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 3. In one embodiment of the anti-CD30L antibody or antigen binding fragment provided herein, including in Section 2 and this Section (Section 4.1), the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 2; and/or (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 4. In one embodiment of the anti-CD30L antibody or antigen binding fragment provided herein, including in Section 2 and this Section (Section 4.1), the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 5; and/or (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 7. In one embodiment of the anti-CD30L antibody or antigen binding fragment provided herein, including in Section 2 and this Section (Section 4.1), the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 6; and/or (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 8. In one embodiment of the anti-CD30L antibody or antigen binding fragment provided herein, including in Section 2 and this Section (Section 4.1), the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 9; and/or (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 11. In one embodiment of the anti-CD30L antibody or antigen binding fragment provided herein, including in Section 2 and this Section (Section 4.1), the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 10; and/or (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 12. In one embodiment of the anti-CD30L antibody or antigen binding fragment provided herein, including in Section 2 and this Section (Section 4.1), the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 13; and/or (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 15. In one embodiment of the anti-CD30L antibody or antigen binding fragment provided herein, including in Section 2 and this Section (Section 4.1), the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 14; and/or (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 16. In one embodiment of the anti-CD30L antibody or antigen binding fragment provided herein, including in Section 2 and this Section (Section 4.1), the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 23; and/or (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 24. In one embodiment of the anti-CD30L antibody or antigen binding fragment provided herein, including in Section 2 and this Section (Section 4.1), the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 25; and/or (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 26. In one embodiment of the anti-CD30L antibody or antigen binding fragment provided herein, including in Section 2 and this Section (Section 4.1), the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 27; and/or (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 28. In one embodiment of the anti-CD30L antibody or antigen binding fragment provided herein, including in Section 2 and this Section (Section 4.1), the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 29; and/or (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 30. In one embodiment of the anti-CD30L antibody or antigen binding fragment provided herein, including in Section 2 and this Section (Section 4.1), the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 31; and/or (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 32.

Furthermore, in one embodiment of the anti-CD30L antibody or antigen binding fragment provided herein, including in Section 2 and this Section (Section 4.1), the antibody or antigen binding fragment thereof comprises: (a) a VH comprising an amino acid sequence set forth in any one of SEQ ID NOs: 1, 2, 5, 6, 9, 10, 13, 14, 17, 19, 21, 23, 25, 27, 29, and 31; and/or (b) a VL comprising an amino acid sequence set forth in any one of SEQ ID NOs: 3, 4, 7, 8, 11, 12, 15, 16, 18, 20, 22, 24, 26, 28, and 30. In one embodiment of the anti-CD30L antibody or antigen binding fragment provided herein, including in Section 2 and this Section (Section 4.1), the antibody or antigen binding fragment thereof comprises: (a) a VH comprising an amino acid sequence set forth in SEQ ID NO: 1; and/or (b) a VL comprising an amino acid sequence set forth in SEQ ID NO: 3. In one embodiment of the anti-CD30L antibody or antigen binding fragment provided herein, including in Section 2 and this Section (Section 4.1), the antibody or antigen binding fragment thereof comprises: (a) a VH comprising an amino acid sequence set forth in SEQ ID NO: 2; and/or (b) a VL comprising an amino acid sequence set forth in SEQ ID NO: 4. In one embodiment of the anti-CD30L antibody or antigen binding fragment provided herein, including in Section 2 and this Section (Section 4.1), the antibody or antigen binding fragment thereof comprises: (a) a VH comprising an amino acid sequence set forth in SEQ ID NO: 5; and/or (b) a VL comprising an amino acid sequence set forth in SEQ ID NO: 7. In one embodiment of the anti-CD30L antibody or antigen binding fragment provided herein, including in Section 2 and this Section (Section 4.1), the antibody or antigen binding fragment thereof comprises: (a) a VH comprising an amino acid sequence set forth in SEQ ID NO: 6; and/or (b) a VL comprising an amino acid sequence set forth in SEQ ID NO: 8. In one embodiment of the anti-CD30L antibody or antigen binding fragment provided herein, including in Section 2 and this Section (Section 4.1), the antibody or antigen binding fragment thereof comprises: (a) a VH comprising an amino acid sequence set forth in SEQ ID NO: 9; and/or (b) a VL comprising an amino acid sequence set forth in SEQ ID NO: 11. In one embodiment of the anti-CD30L antibody or antigen binding fragment provided herein, including in Section 2 and this Section (Section 4.1), the antibody or antigen binding fragment thereof comprises: (a) a VH comprising an amino acid sequence set forth in SEQ ID NO: 10; and/or (b) a VL comprising an amino acid sequence set forth in SEQ ID NO: 12. In one embodiment of the anti-CD30L antibody or antigen binding fragment provided herein, including in Section 2 and this Section (Section 4.1), the antibody or antigen binding fragment thereof comprises: (a) a VH comprising an amino acid sequence set forth in SEQ ID NO: 13; and/or (b) a VL comprising an amino acid sequence set forth in SEQ ID NO: 15. In one embodiment of the anti-CD30L antibody or antigen binding fragment provided herein, including in Section 2 and this Section (Section 4.1), the antibody or antigen binding fragment thereof comprises: (a) a VH comprising an amino acid sequence set forth in SEQ ID NO: 14; and/or (b) a VL comprising an amino acid sequence set forth in SEQ ID NO: 16. In one embodiment of the anti-CD30L antibody or antigen binding fragment provided herein, including in Section 2 and this Section (Section 4.1), the antibody or antigen binding fragment thereof comprises: (a) a VH comprising an amino acid sequence set forth in SEQ ID NO: 23; and/or (b) a VL comprising an amino acid sequence set forth in SEQ ID NO: 24. In one embodiment of the anti-CD30L antibody or antigen binding fragment provided herein, including in Section 2 and this Section (Section 4.1), the antibody or antigen binding fragment thereof comprises: (a) a VH comprising an amino acid sequence set forth in SEQ ID NO: 25; and/or (b) a VL comprising an amino acid sequence set forth in SEQ ID NO: 26. In one embodiment of the anti-CD30L antibody or antigen binding fragment provided herein, including in Section 2 and this Section (Section 4.1), the antibody or antigen binding fragment thereof comprises: (a) a VH comprising an amino acid sequence set forth in SEQ ID NO: 27; and/or (b) a VL comprising an amino acid sequence set forth in SEQ ID NO: 28. In one embodiment of the anti-CD30L antibody or antigen binding fragment provided herein, including in Section 2 and this Section (Section 4.1), the antibody or antigen binding fragment thereof comprises: (a) a VH comprising an amino acid sequence set forth in SEQ ID NO: 29; and/or (b) a VL comprising an amino acid sequence set forth in SEQ ID NO: 30. In one embodiment of the anti-CD30L antibody or antigen binding fragment provided herein, including in Section 2 and this Section (Section 4.1), the antibody or antigen binding fragment thereof comprises: (a) a VH comprising an amino acid sequence set forth in SEQ ID NO: 31; and/or (b) a VL comprising an amino acid sequence set forth in SEQ ID NO: 32.

The CDRs of the anti-CD30L antibody or antigen binding fragment provided herein are also provided by the CDR definitions as described herein (Kabat, Chothia, AbM, Contact, IMGT, and Aho) for the provided VH and VL sequences. Accordingly, in one embodiment of the anti-CD30L antibody or antigen binding fragment provided herein, including in Section 2 and this Section (Section 4.1), the antibody or antigen binding fragment thereof comprises: (a) the CDR-H1, CDR-H2, and CDR-H3 of the corresponding CDR-H1, CDR-H2, and CDR-H3 of the VH sequence set forth in SEQ ID NO: 1; and/or (b) the CDR-L1, CDR-L2, and CDR-L3 of the corresponding CDR-L1, CDR-L2, and CDR-L3 of the VL sequence set forth in SEQ ID NO: 3, wherein the CDRs are determined by Kabat, Chothia, AbM, Contact, IMGT, or Aho numbering scheme. In one embodiment of the anti-CD30L antibody or antigen binding fragment provided herein, including in Section 2 and this Section (Section 4.1), the antibody or antigen binding fragment thereof comprises: (a) the CDR-H1, CDR-H2, and CDR-H3 of the corresponding CDR-H1, CDR-H2, and CDR-H3 of the VH sequence set forth in SEQ ID NO: 2; and/or (b) the CDR-L1, CDR-L2, and CDR-L3 of the corresponding CDR-L1, CDR-L2, and CDR-L3 of the VL sequence set forth in SEQ ID NO: 4, wherein the CDRs are determined by Kabat, Chothia, AbM, Contact, IMGT, or Aho numbering scheme. In one embodiment of the anti-CD30L antibody or antigen binding fragment provided herein, including in Section 2 and this Section (Section 4.1), the antibody or antigen binding fragment thereof comprises: (a) the CDR-H1, CDR-H2, and CDR-H3 of the corresponding CDR-H1, CDR-H2, and CDR-H3 of the VH sequence set forth in SEQ ID NO: 5; and/or (b) the CDR-L1, CDR-L2, and CDR-L3 of the corresponding CDR-L1, CDR-L2, and CDR-L3 of the VL sequence set forth in SEQ ID NO: 7, wherein the CDRs are determined by Kabat, Chothia, AbM, Contact, IMGT, or Aho numbering scheme. In one embodiment of the anti-CD30L antibody or antigen binding fragment provided herein, including in Section 2 and this Section (Section 4.1), the antibody or antigen binding fragment thereof comprises: (a) the CDR-H1, CDR-H2, and CDR-H3 of the corresponding CDR-H1, CDR-H2, and CDR-H3 of the VH sequence set forth in SEQ ID NO: 6; and/or (b) the CDR-L1, CDR-L2, and CDR-L3 of the corresponding CDR-L1, CDR-L2, and CDR-L3 of the VL sequence set forth in SEQ ID NO: 8, wherein the CDRs are determined by Kabat, Chothia, AbM, Contact, IMGT, or Aho numbering scheme. In one embodiment of the anti-CD30L antibody or antigen binding fragment provided herein, including in Section 2 and this Section (Section 4.1), the antibody or antigen binding fragment thereof comprises: (a) the CDR-H1, CDR-H2, and CDR-H3 of the corresponding CDR-H1, CDR-H2, and CDR-H3 of the VH sequence set forth in SEQ ID NO: 9; and/or (b) the CDR-L1, CDR-L2, and CDR-L3 of the corresponding CDR-L1, CDR-L2, and CDR-L3 of the VL sequence set forth in SEQ ID NO: 11, wherein the CDRs are determined by Kabat, Chothia, AbM, Contact, IMGT, or Aho numbering scheme. In one embodiment of the anti-CD30L antibody or antigen binding fragment provided herein, including in Section 2 and this Section (Section 4.1), the antibody or antigen binding fragment thereof comprises: (a) the CDR-H1, CDR-H2, and CDR-H3 of the corresponding CDR-H1, CDR-H2, and CDR-H3 of the VH sequence set forth in SEQ ID NO: 10; and/or (b) the CDR-L1, CDR-L2, and CDR-L3 of the corresponding CDR-L1, CDR-L2, and CDR-L3 of the VL sequence set forth in SEQ ID NO: 12, wherein the CDRs are determined by Kabat, Chothia, AbM, Contact, IMGT, or Aho numbering scheme. In one embodiment of the anti-CD30L antibody or antigen binding fragment provided herein, including in Section 2 and this Section (Section 4.1), the antibody or antigen binding fragment thereof comprises: (a) the CDR-H1, CDR-H2, and CDR-H3 of the corresponding CDR-H1, CDR-H2, and CDR-H3 of the VH sequence set forth in SEQ ID NO: 13; and/or (b) the CDR-L1, CDR-L2, and CDR-L3 of the corresponding CDR-L1, CDR-L2, and CDR-L3 of the VL sequence set forth in SEQ ID NO: 15, wherein the CDRs are determined by Kabat, Chothia, AbM, Contact, IMGT, or Aho numbering scheme. In one embodiment of the anti-CD30L antibody or antigen binding fragment provided herein, including in Section 2 and this Section (Section 4.1), the antibody or antigen binding fragment thereof comprises: (a) the CDR-H1, CDR-H2, and CDR-H3 of the corresponding CDR-H1, CDR-H2, and CDR-H3 of the VH sequence set forth in SEQ ID NO: 14; and/or (b) the CDR-L1, CDR-L2, and CDR-L3 of the corresponding CDR-L1, CDR-L2, and CDR-L3 of the VL sequence set forth in SEQ ID NO: 16, wherein the CDRs are determined by Kabat, Chothia, AbM, Contact, IMGT, or Aho numbering scheme. In one embodiment of the anti-CD30L antibody or antigen binding fragment provided herein, including in Section 2 and this Section (Section 4.1), the antibody or antigen binding fragment thereof comprises: (a) the CDR-H1, CDR-H2, and CDR-H3 of the corresponding CDR-H1, CDR-H2, and CDR-H3 of the VH sequence set forth in SEQ ID NO: 23; and/or (b) the CDR-L1, CDR-L2, and CDR-L3 of the corresponding CDR-L1, CDR-L2, and CDR-L3 of the VL sequence set forth in SEQ ID NO: 24, wherein the CDRs are determined by Kabat, Chothia, AbM, Contact, IMGT, or Aho numbering scheme. In one embodiment of the anti-CD30L antibody or antigen binding fragment provided herein, including in Section 2 and this Section (Section 4.1), the antibody or antigen binding fragment thereof comprises: (a) the CDR-H1, CDR-H2, and CDR-H3 of the corresponding CDR-H1, CDR-H2, and CDR-H3 of the VH sequence set forth in SEQ ID NO: 25; and/or (b) the CDR-L1, CDR-L2, and CDR-L3 of the corresponding CDR-L1, CDR-L2, and CDR-L3 of the VL sequence set forth in SEQ ID NO: 26, wherein the CDRs are determined by Kabat, Chothia, AbM, Contact, IMGT, or Aho numbering scheme. In one embodiment of the anti-CD30L antibody or antigen binding fragment provided herein, including in Section 2 and this Section (Section 4.1), the antibody or antigen binding fragment thereof comprises: (a) the CDR-H1, CDR-H2, and CDR-H3 of the corresponding CDR-H1, CDR-H2, and CDR-H3 of the VH sequence set forth in SEQ ID NO: 27; and/or (b) the CDR-L1, CDR-L2, and CDR-L3 of the corresponding CDR-L1, CDR-L2, and CDR-L3 of the VL sequence set forth in SEQ ID NO: 28, wherein the CDRs are determined by Kabat, Chothia, AbM, Contact, IMGT, or Aho numbering scheme. In one embodiment of the anti-CD30L antibody or antigen binding fragment provided herein, including in Section 2 and this Section (Section 4.1), the antibody or antigen binding fragment thereof comprises: (a) the CDR-H1, CDR-H2, and CDR-H3 of the corresponding CDR-H1, CDR-H2, and CDR-H3 of the VH sequence set forth in SEQ ID NO: 29; and/or (b) the CDR-L1, CDR-L2, and CDR-L3 of the corresponding CDR-L1, CDR-L2, and CDR-L3 of the VL sequence set forth in SEQ ID NO: 30, wherein the CDRs are determined by Kabat, Chothia, AbM, Contact, IMGT, or Aho numbering scheme. In one embodiment of the anti-CD30L antibody or antigen binding fragment provided herein, including in Section 2 and this Section (Section 4.1), the antibody or antigen binding fragment thereof comprises: (a) the CDR-H1, CDR-H2, and CDR-H3 of the corresponding CDR-H1, CDR-H2, and CDR-H3 of the VH sequence set forth in SEQ ID NO: 31; and/or (b) the CDR-L1, CDR-L2, and CDR-L3 of the corresponding CDR-L1, CDR-L2, and CDR-L3 of the VL sequence set forth in SEQ ID NO: 32, wherein the CDRs are determined by Kabat, Chothia, AbM, Contact, IMGT, or Aho numbering scheme.

As is clear from the descriptions above and below, the anti-CD30L antibodies provided herein can further comprise constant regions. In one embodiment of the anti-CD30L antibody or antigen binding fragment provided herein, including in Section 2 and this Section (Section 4.1), the antibody or antigen binding fragment thereof further comprises an immunoglobulin heavy chain constant region. In one embodiment of the anti-CD30L antibody or antigen binding fragment provided herein, including in Section 2 and this Section (Section 4.1), the antibody or antigen binding fragment thereof further comprises an immunoglobulin light chain constant region. In one embodiment of the anti-CD30L antibody or antigen binding fragment provided herein, including in Section 2 and this Section (Section 4.1), the antibody or antigen binding fragment thereof further comprises an immunoglobulin heavy chain constant region and an immunoglobulin light chain constant region. In one embodiment, the heavy chain constant region is an IgG heavy chain constant region. In one embodiment, the light chain constant region is an IgG light chain constant region. In one embodiment, the light chain constant region is a kappa (κ) chain constant region. In one embodiment, the light chain constant region is a lambda (λ) chain constant region. In one embodiment, the heavy chain constant region is a IgG1 heavy chain constant region. In one embodiment, the heavy chain constant region is a IgG2 heavy chain constant region. In one embodiment, the heavy chain constant region is a IgG3 heavy chain constant region. In one embodiment, the heavy chain constant region is a IgG4 heavy chain constant region. In one embodiment, the constant region comprises an amino acid sequence having about 80, 85, 90, 95, 97, 98, 99, or 100% sequence identity to the amino acid sequence set forth by any one of SEQ ID NOs: 500-512. In one embodiment, the constant region comprises an amino acid sequence having about 80, 81, 82, 83, 84, 85, 86, 87, 88, 89, 90, 91, 92, 93, 94, 95, 96, 97, 98, 99% sequence identity to the amino acid sequence set forth by any one of SEQ ID NOs: 500-512. In one embodiment, the constant region comprises the amino acid sequence set forth by any one of SEQ ID NOs: 500-512. In one embodiment, the constant region comprises the amino acid sequence set forth by any one of SEQ ID NO: 500. In one embodiment, the constant region comprises the amino acid sequence set forth by any one of SEQ ID NO: 501. In one embodiment, the constant region comprises the amino acid sequence set forth by any one of SEQ ID NO: 502. In one embodiment, the constant region comprises the amino acid sequence set forth by any one of SEQ ID NO: 503. In one embodiment, the constant region comprises the amino acid sequence set forth by any one of SEQ ID NO: 504. In one embodiment, the constant region comprises the amino acid sequence set forth by any one of SEQ ID NO: 505. In one embodiment, the constant region comprises the amino acid sequence set forth by any one of SEQ ID NO: 506. In one embodiment, the constant region comprises the amino acid sequence set forth by any one of SEQ ID NO: 507. In one embodiment, the constant region comprises the amino acid sequence set forth by any one of SEQ ID NO: 508. In one embodiment, the constant region comprises the amino acid sequence set forth by any one of SEQ ID NO: 509. In one embodiment, the constant region comprises the amino acid sequence set forth by any one of SEQ ID NO: 510. In one embodiment, the constant region comprises the amino acid sequence set forth by any one of SEQ ID NO: 511. In one embodiment, the constant region comprises the amino acid sequence set forth by any one of SEQ ID NO: 512.

The disclosure further provides anti-CD30L antibody or antigen binding fragments that bind to murine CD30L, functionally block both the murine CD30-CD30L interaction, and/or functionally block the CD30L-mediated CD30 signaling and IL-8 releasing in murine cells. Accordingly, in one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in any one of SEQ ID NOs: 774-779; (b) a CDR-H2 comprising the amino acid sequence set forth in any one of SEQ ID NOs: 780-785; (c) a CDR-H3 comprising the amino acid sequence set forth in any one of SEQ ID NOs: 786-791; (d) a CDR-L1 comprising the amino acid sequence set forth in any one of SEQ ID NOs: 792-797; (e) a CDR-L2 comprising the amino acid sequence set forth in any one of SEQ ID NOs: 798-803; and/or (f) a CDR-L3 comprising the amino acid sequence set forth in any one of SEQ ID NOs: 804-809. In one embodiment of the anti-CD30L antibody or antigen binding fragment provided herein, including in Section 2 and this Section (Section 4.1), the antibody or antigen binding fragment thereof comprises: (a) the CDR-H1, CDR-H2, and CDR-H3 of the corresponding CDR-H1, CDR-H2, and CDR-H3 of the VH sequence set forth in SEQ ID NO: 770; and/or (b) the CDR-L1, CDR-L2, and CDR-L3 of the corresponding CDR-L1, CDR-L2, and CDR-L3 of the VL sequence set forth in SEQ ID NO: 770, wherein the CDRs are determined by Kabat, Chothia, AbM, Contact, IMGT, or Aho numbering scheme.

Specifically, in one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 774; (b) a CDR-H2 comprising the amino acid sequence set forth in SEQ ID NO: 780; (c) a CDR-H3 comprising the amino acid sequence set forth in SEQ ID NO: 786; (d) a CDR-L1 comprising the amino acid sequence set forth in SEQ ID NO: 792; (e) a CDR-L2 comprising the amino acid sequence set forth SEQ ID NO: 798; and/or (f) a CDR-L3 comprising the amino acid sequence set forth SEQ ID NO: 804. In another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 775; (b) a CDR-H2 comprising the amino acid sequence set forth in SEQ ID NO: 781; (c) a CDR-H3 comprising the amino acid sequence set forth in SEQ ID NO: 787; (d) a CDR-L1 comprising the amino acid sequence set forth in SEQ ID NO: 793; (e) a CDR-L2 comprising the amino acid sequence set forth SEQ ID NO: 799; and/or (f) a CDR-L3 comprising the amino acid sequence set forth SEQ ID NO: 805. In yet another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 776; (b) a CDR-H2 comprising the amino acid sequence set forth in SEQ ID NO: 782; (c) a CDR-H3 comprising the amino acid sequence set forth in SEQ ID NO: 788; (d) a CDR-L1 comprising the amino acid sequence set forth in SEQ ID NO: 794; (e) a CDR-L2 comprising the amino acid sequence set forth SEQ ID NO: 800; and/or (f) a CDR-L3 comprising the amino acid sequence set forth SEQ ID NO: 806. In one embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 777; (b) a CDR-H2 comprising the amino acid sequence set forth in SEQ ID NO: 783; (c) a CDR-H3 comprising the amino acid sequence set forth in SEQ ID NO: 789; (d) a CDR-L1 comprising the amino acid sequence set forth in SEQ ID NO: 795; (e) a CDR-L2 comprising the amino acid sequence set forth SEQ ID NO: 801; and/or (f) a CDR-L3 comprising the amino acid sequence set forth SEQ ID NO: 807. In another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 778; (b) a CDR-H2 comprising the amino acid sequence set forth in SEQ ID NO: 784; (c) a CDR-H3 comprising the amino acid sequence set forth in SEQ ID NO: 790; (d) a CDR-L1 comprising the amino acid sequence set forth in SEQ ID NO: 796; (e) a CDR-L2 comprising the amino acid sequence set forth SEQ ID NO: 802; and/or (f) a CDR-L3 comprising the amino acid sequence set forth SEQ ID NO: 808. In yet another embodiment of the anti-CD30L antibody or antigen binding fragment provided herein, including in Section 2 and this Section (Section 4.1), the antibody or antigen binding fragment thereof comprises: (a) a CDR-H1 comprising the amino acid sequence set forth in SEQ ID NO: 779; (b) a CDR-H2 comprising the amino acid sequence set forth in SEQ ID NO: 785; (c) a CDR-H3 comprising the amino acid sequence set forth in SEQ ID NO: 791; (d) a CDR-L1 comprising the amino acid sequence set forth in SEQ ID NO: 797; (e) a CDR-L2 comprising the amino acid sequence set forth SEQ ID NO: 803; and/or (f) a CDR-L3 comprising the amino acid sequence set forth SEQ ID NO: 809.

Additionally, the disclosure provides that the anti-CD30L antibodies or antigen binding fragments provided herein can comprise the combination of immunoglobulin heavy chain variable region (VH) and immunoglobulin light chain variable region (VL) of the antibody or antigen binding fragments that bind to and block murine CD30L. In one embodiment of the anti-CD30L antibody or antigen binding fragment provided herein, including in Section 2 and this Section (Section 4.1), the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 770; and (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 771. In one embodiment of the anti-CD30L antibody or antigen binding fragment provided herein, including in Section 2 and this Section (Section 4.1), the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 770; or (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 771. In one embodiment of the anti-CD30L antibody or antigen binding fragment provided herein, including in Section 2 and this Section (Section 4.1), the antibody or antigen binding fragment thereof comprises: (a) a VH comprising an amino acid sequence set forth in SEQ ID NO: 770; and (b) a VL comprising an amino acid set forth in SEQ ID NO: 771. In one embodiment of the anti-CD30L antibody or antigen binding fragment provided herein, including in Section 2 and this Section (Section 4.1), the antibody or antigen binding fragment thereof comprises: (a) a VH comprising an amino acid sequence set forth in SEQ ID NO: 770; or (b) a VL comprising an amino acid set forth in SEQ ID NO: 771.

Similarly, the disclosure provides that the anti-CD30L antibodies provided herein can comprise the combination of immunoglobulin heavy chain (HC) and immunoglobulin light chain (LC) of the antibodies that bind to and block murine CD30L. In one embodiment of the anti-CD30L antibody provided herein, including in Section 2 and this Section (Section 4.1), the antibody comprises: (a) a HC comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 772; and (b) a LC comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 773. In one embodiment of the anti-CD30L antibody provided herein, including in Section 2 and this Section (Section 4.1), the antibody comprises: (a) a HC comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 772; or (b) a LC comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 773. In one embodiment of the anti-CD30L antibody provided herein, including in Section 2 and this Section (Section 4.1), the antibody comprises: (a) a HC comprising an amino acid sequence set forth in SEQ ID NO: 772; and (b) a LC comprising an amino acid set forth in SEQ ID NO: 773. In one embodiment of the anti-CD30L antibody provided herein, including in Section 2 and this Section (Section 4.1), the antibody comprises: (a) a HC comprising an amino acid sequence set forth in SEQ ID NO: 772; or (b) a LC comprising an amino acid set forth in SEQ ID NO: 773.

The antibody or antigen binding fragment thereof provided herein can have various functional properties as described herein, e.g., in Section 5 (EXAMPLES). Accordingly, in one embodiment of the anti-CD30L antibody or antigen binding fragment provided herein, including in Section 2 and this Section (Section 4.1), the antibody or antigen binding fragment thereof blocks a binding interaction between CD30L and CD30. In one embodiment of the anti-CD30L antibody or antigen binding fragment provided herein, including in Section 2 and this Section (Section 4.1), the antibody or antigen binding fragment thereof inhibits a binding interaction between CD30L and CD30. In one embodiment, the interaction between CD30L and CD30 for assessing the blocking or inhibition is determined in an ELISA assay, a cell binding assay with CD30L expressing cells, a KinExA assay, or a surface plasmon resonance (SPR) assay. In one embodiment, the interaction between CD30L and CD30 for assessing the blocking or inhibition is determined in an ELISA assay. In one embodiment, the interaction between CD30L and CD30 for assessing the blocking or inhibition is determined in a cell binding assay with CD30L expressing cells. In one embodiment, the interaction between CD30L and CD30 for assessing the blocking or inhibition is determined in a KinExA assay. In one embodiment, the interaction between CD30L and CD30 for assessing the blocking or inhibition is determined in an SPR assay.

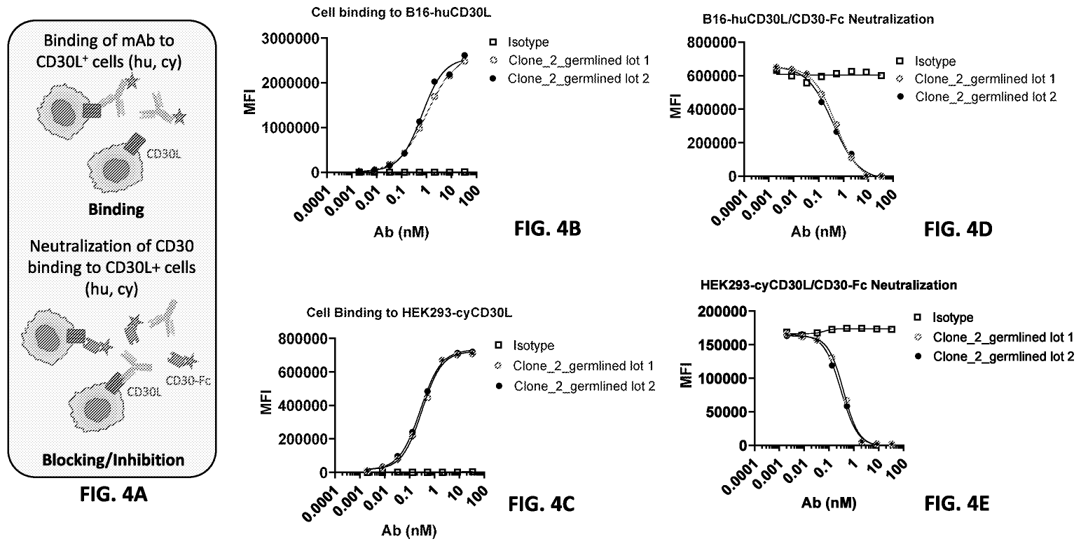

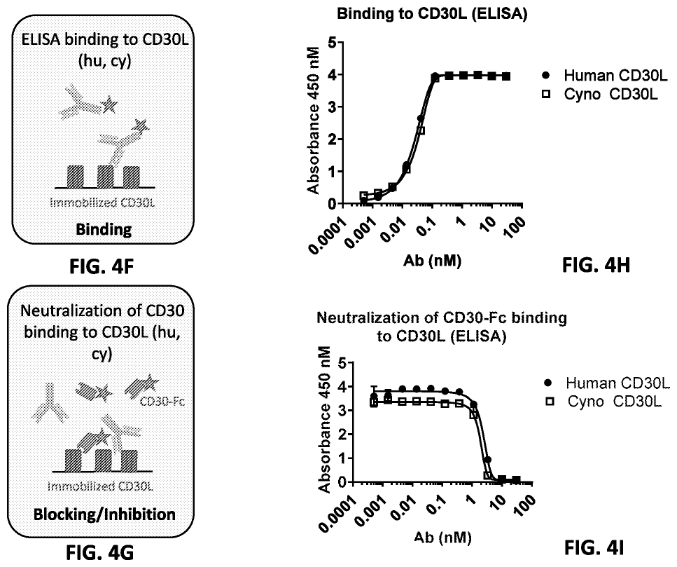

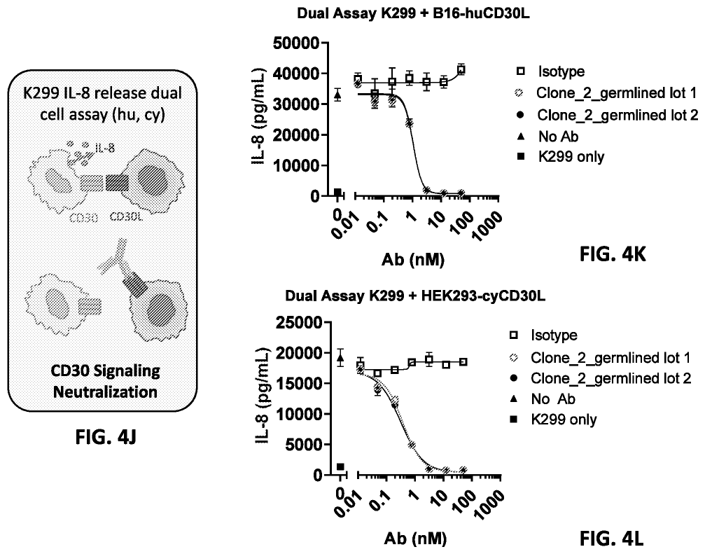

As shown in Example 8, the anti-CD30L or antigen binding fragment provided herein have high binding specificity for CD30L, binding specifically to CD30L among 6,232 targets tested. Accordingly, in one embodiment of the anti-CD30L antibody or antigen binding fragment provided herein, including in Section 2 and this Section (Section 4.1), the antibody or antigen binding fragment thereof specifically binds to CD30L. In some embodiments, the ELISA, SPR, KinExA, and/or cell binding assay with CD30L expressing cells are as described and demonstrated in Section 5 (EXAMPLES) (e.g. FIGS. 4A, 4F, and 4G).

As further demonstrated herein, e.g. in Section 5 (EXAMPLES), the anti-CD30L antibody provided herein can block or inhibit CD30L-mediated CD30 signaling in cell-based assays. Such cell assays include cytokine (e.g. IL-8) release dual cell assays. Briefly and using IL-8 release as an example of cytokine release, when co-cultured with CD30L expressing cells (e.g. B16 cells expressing humanCD30L or HEK293 expressing cynoCD30L), K299 CD30+ cells release IL-8 upon ligation with and stimulation by the co-cultured CD30L expressing cells (FIG. 4J). Blocking of the CD30L-CD30 interaction can proportionally reduce the release of IL-8 and thus the amount of IL-8 released can be used as a readout for the effectiveness of antibody mediated blocking of CD30L-CD30 interactions between the co-cultured cells. In some embodiments, provided herein are antibodies or antigen binding fragments that (i) bind to CD30L and (ii) block or inhibit CD30L-mediated CD30 signaling in cell-based assays described herein.

Accordingly, in one embodiment of the anti-CD30L antibody or antigen binding fragment provided herein, including in Section 2 and this Section (Section 4.1), the antibody or antigen binding fragment thereof inhibits interleukin-8 secretion in a cell-based assay. In one embodiment of the anti-CD30L antibody or antigen binding fragment provided herein, including in Section 2 and this Section (Section 4.1), the antibody or antigen binding fragment thereof inhibits interleukin-6 secretion in a cell-based assay. In one embodiment of the anti-CD30L antibody or antigen binding fragment provided herein, including in Section 2 and this Section (Section 4.1), the antibody or antigen binding fragment thereof inhibits both interleukin-8 and interleukin-6 secretion in a cell-based assay. In one embodiment of the anti-CD30L antibody or antigen binding fragment provided herein, including in Section 2 and this Section (Section 4.1), the antibody or antigen binding fragment thereof blocks interleukin-8 secretion in a cell-based assay. In one embodiment of the anti-CD30L antibody or antigen binding fragment provided herein, including in Section 2 and this Section (Section 4.1), the antibody or antigen binding fragment thereof blocks interleukin-6 secretion in a cell-based assay. In one embodiment of the anti-CD30L antibody or antigen binding fragment provided herein, including in Section 2 and this Section (Section 4.1), the antibody or antigen binding fragment thereof blocks both interleukin-8 and interleukin-6 secretion in a cell-based assay. In one embodiment of the anti-CD30L antibody or antigen binding fragment provided herein, including in Section 2 and this Section (Section 4.1) such as those of this paragraph, the cell-based assay is a dual cell assay with a cell expressing CD30 and a cell expressing CD30L.

In one embodiment, provided herein is an antibody or antigen binding fragment thereof that inhibits interleukin-8 secretion in a cell-based assay. In one embodiment, provided herein is an antibody or antigen binding fragment thereof that inhibits interleukin-6 secretion in a cell-based assay. In one embodiment, provided herein is an antibody or antigen binding fragment thereof that inhibits both interleukin-8 and interleukin-6 secretion in a cell-based assay. In one embodiment, provided herein is an antibody or antigen binding fragment thereof that blocks interleukin-8 secretion in a cell-based assay. In one embodiment, provided herein is an antibody or antigen binding fragment thereof that blocks interleukin-6 secretion in a cell-based assay. In one embodiment, provided herein is an antibody or antigen binding fragment thereof that blocks both interleukin-8 and interleukin-6 secretion in a cell-based assay. In one embodiment of the anti-CD30L antibody or antigen binding fragment provided herein, including in Section 2 and this Section (Section 4.1) such as those of this paragraph, the cell-based assay is a dual cell assay with a cell expressing CD30 and a cell expressing CD30L.

Similarly, in one embodiment, provided herein is an anti-CD30L antibody or antigen binding fragment that reduces IBD induced weight loss. In another embodiment, provided herein is an anti-CD30L antibody or antigen binding fragment that reduces CD induced weight loss. In yet another embodiment, provided herein is an anti-CD30L antibody or antigen binding fragment that reduces UC induced weight loss. In one embodiment of the anti-CD30L antibody or antigen binding fragment provided herein, including in Section 2 and this Section (Section 4.1), the antibody or antigen binding fragment thereof reduces IBD induced weight loss. In one embodiment of the anti-CD30L antibody or antigen binding fragment provided herein, including in Section 2 and this Section (Section 4.1), the antibody or antigen binding fragment thereof reduces CD induced weight loss. In one embodiment of the anti-CD30L antibody or antigen binding fragment provided herein, including in Section 2 and this Section (Section 4.1), the antibody or antigen binding fragment thereof reduces UC induced weight loss.

Additionally, in one embodiment, provided herein is an anti-CD30L antibody or antigen binding fragment that reduces IBD induced injury to intestinal mucosal barrier. In another embodiment, provided herein is an anti-CD30L antibody or antigen binding fragment that reduces CD induced injury to intestinal mucosal barrier. In yet another embodiment, provided herein is an anti-CD30L antibody or antigen binding fragment that reduces UC induced injury to intestinal mucosal barrier. In one embodiment of the anti-CD30L antibody or antigen binding fragment provided herein, including in Section 2 and this Section (Section 4.1), the antibody or antigen binding fragment thereof reduces IBD induced injury to intestinal mucosal barrier. In one embodiment of the anti-CD30L antibody or antigen binding fragment provided herein, including in Section 2 and this Section (Section 4.1), the antibody or antigen binding fragment thereof reduces CD induced injury to intestinal mucosal barrier. In one embodiment of the anti-CD30L antibody or antigen binding fragment provided herein, including in Section 2 and this Section (Section 4.1), the antibody or antigen binding fragment thereof reduces UC induced injury to intestinal mucosal barrier.

Alternatively, in one embodiment, provided herein is an anti-CD30L antibody or antigen binding fragment that reduces IBD induced intestinal histopathology sum score. In another embodiment, provided herein is an anti-CD30L antibody or antigen binding fragment that reduces CD induced intestinal histopathology sum score. In yet another embodiment, provided herein is an anti-CD30L antibody or antigen binding fragment that reduces UC induced intestinal histopathology sum score. In one embodiment of the anti-CD30L antibody or antigen binding fragment provided herein, including in Section 2 and this Section (Section 4.1), the antibody or antigen binding fragment thereof reduces IBD induced intestinal histopathology sum score. In one embodiment of the anti-CD30L antibody or antigen binding fragment provided herein, including in Section 2 and this Section (Section 4.1), the antibody or antigen binding fragment thereof reduces CD induced intestinal histopathology sum score. In one embodiment of the anti-CD30L antibody or antigen binding fragment provided herein, including in Section 2 and this Section (Section 4.1), the antibody or antigen binding fragment thereof reduces UC induced intestinal histopathology sum score.

Similarly, in one embodiment, provided herein is an anti-CD30L antibody or antigen binding fragment that reduces IBD induced endoscopy score. In another embodiment, provided herein is an anti-CD30L antibody or antigen binding fragment that reduces CD induced endoscopy score. In yet another embodiment, provided herein is an anti-CD30L antibody or antigen binding fragment that reduces UC induced endoscopy score. In one embodiment of the anti-CD30L antibody or antigen binding fragment provided herein, including in Section 2 and this Section (Section 4.1), the antibody or antigen binding fragment thereof reduces IBD induced endoscopy score. In one embodiment of the anti-CD30L antibody or antigen binding fragment provided herein, including in Section 2 and this Section (Section 4.1), the antibody or antigen binding fragment thereof reduces CD induced endoscopy score. In one embodiment of the anti-CD30L antibody or antigen binding fragment provided herein, including in Section 2 and this Section (Section 4.1), the antibody or antigen binding fragment thereof reduces UC induced endoscopy score.

The disclosure demonstrated in Section 5 (EXAMPLES) that the anti-CD30L antibody or antigen binding fragment bind to CD30L with strong binding affinity and blocked CD30L with high effectiveness. Accordingly, in one embodiment of the anti-CD30L antibody or antigen binding fragment provided herein, including in Section 2 and this Section (Section 4.1) such as those of this paragraph, the antibody or antigen binding fragment thereof binds to CD30L with a dissociation equilibrium constant (K_{D}) of no more than 60, no more than 65, no more than 70, no more than 75, no more than 80, no more than 85, no more than 90, no more than 95, no more than 100, no more than 105, no more than 110, no more than 115, no more than 120, no more than 125, no more than 130, no more than 135, no more than 140, no more than 145, no more than 150, no more than 155, no more than 160, no more than 165, no more than 170, no more than 175, no more than 180, no more than 185, no more than 190, no more than 195, no more than 200, no more than 250, no more than 300, no more than 350, no more than 400, no more than 450, no more than 500, no more than 550, no more than 600, no more than 650, no more than 700, no more than 750, no more than 800, no more than 850, no more than 900, no more than 950, or no more than 1000 pM. In one embodiment of the anti-CD30L antibody or antigen binding fragment provided herein, including in Section 2 and this Section (Section 4.1) such as those of this paragraph, the antibody or antigen binding fragment thereof binds to CD30L with a dissociation equilibrium constant (K_{D}) of about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 250, about 300, about 350, about 400, about 450, about 500, about 550, about 600, about 650, about 700, about 750, about 800, about 850, about 900, about 950, or about 1000 pM. In one embodiment of the anti-CD30L antibody or antigen binding fragment provided herein, including in Section 2 and this Section (Section 4.1) such as those of this paragraph, the antibody or antigen binding fragment thereof binds to CD30L with a K_{D} of no more than 90.5 pM. In one embodiment of the anti-CD30L antibody or antigen binding fragment provided herein, including in Section 2 and this Section (Section 4.1) such as those of this paragraph, the antibody or antigen binding fragment thereof binds to CD30L with a K_{D} of no more than 125 pM. In one embodiment of the anti-CD30L antibody or antigen binding fragment provided herein, including in Section 2 and this Section (Section 4.1) such as those of this paragraph, the antibody or antigen binding fragment thereof binds to CD30L with a K_{D} of no more than 65.4 pM. In one embodiment of the anti-CD30L antibody or antigen binding fragment provided herein, including in Section 2 and this Section (Section 4.1) such as those of this paragraph, the antibody or antigen binding fragment thereof binds to CD30L with a K_{D} of no more than 122 pM. In one embodiment of the anti-CD30L antibody or antigen binding fragment provided herein, including in Section 2 and this Section (Section 4.1) such as those of this paragraph, the antibody or antigen binding fragment thereof binds to CD30L with a K_{D} of no more than 51.3 pM. In one embodiment of the anti-CD30L antibody or antigen binding fragment provided herein, including in Section 2 and this Section (Section 4.1) such as those of this paragraph, the antibody or antigen binding fragment thereof binds to CD30L with a K_{D} of no more than 244 pM. In one embodiment of the anti-CD30L antibody or antigen binding fragment provided herein, including in Section 2 and this Section (Section 4.1) such as those of this paragraph, the antibody or antigen binding fragment thereof binds to CD30L with a K_{D} of about 90.5 pM. In one embodiment of the anti-CD30L antibody or antigen binding fragment provided herein, including in Section 2 and this Section (Section 4.1) such as those of this paragraph, the antibody or antigen binding fragment thereof binds to CD30L with a K_{D} of about 125 pM. In one embodiment of the anti-CD30L antibody or antigen binding fragment provided herein, including in Section 2 and this Section (Section 4.1) such as those of this paragraph, the antibody or antigen binding fragment thereof binds to CD30L with a K_{D} of about 64.5 pM. In one embodiment of the anti-CD30L antibody or antigen binding fragment provided herein, including in Section 2 and this Section (Section 4.1) such as those of this paragraph, the antibody or antigen binding fragment thereof binds to CD30L with a K_{D} of about 122 pM. In one embodiment of the anti-CD30L antibody or antigen binding fragment provided herein, including in Section 2 and this Section (Section 4.1) such as those of this paragraph, the antibody or antigen binding fragment thereof binds to CD30L with a K_{D} of about 51.3 pM. In one embodiment of the anti-CD30L antibody or antigen binding fragment provided herein, including in Section 2 and this Section (Section 4.1) such as those of this paragraph, the antibody or antigen binding fragment thereof binds to CD30L with a K_{D} of about 244 pM. In one embodiment of the anti-CD30L antibody or antigen binding fragment provided herein, including in Section 2 and this Section (Section 4.1) such as those of this paragraph, the antibody or antigen binding fragment thereof binds to CD30L with a K_{D} of about 65 to about 125 pM. In one embodiment of the anti-CD30L antibody or antigen binding fragment provided herein, including in Section 2 and this Section (Section 4.1) such as those of this paragraph, the antibody or antigen binding fragment thereof binds to CD30L with a K_{D} of about 50 to about 140 pM, about 55 to about 135 pM, about 60 to about 130 pM, about 65 to about 125 pM, about 70 to about 120 pM, about 75 to about 115 pM, about 80 to about 110 pM, about 85 to about 105 pM, about 90 to about 100 pM, or about 95 to about 100 pM. In one embodiment of the anti-CD30L antibody or antigen binding fragment provided herein, including in Section 2 and this Section (Section 4.1) such as those of this paragraph, the antibody or antigen binding fragment thereof binds to CD30L with a K_{D} of about 51 to about 244 pM. In one embodiment of the anti-CD30L antibody or antigen binding fragment provided herein, including in Section 2 and this Section (Section 4.1) such as those of this paragraph, the antibody or antigen binding fragment thereof binds to CD30L with a K_{D} of about 50 to about 250 pM, about 51 to about 244 pM, about 55 to about 240 pM, about 60 to about 235 pM, about 65 to about 230 pM, about 70 to about 225 pM, about 75 to about 220 pM, about 80 to about 215 pM, about 80 to about 210 pM, about 85 to about 205 pM, about 90 to about 200 pM, about 95 to about 195 pM, about 100 to about 190 pM, about 105 to about 185 pM, about 110 to about 180 pM, about 115 to about 175 pM, about 120 to about 170 pM, about 125 to about 165 pM, about 130 to about 160 pM, about 135 to about 155 pM, or about 140 to about 150 pM.

Additionally, in one embodiment of the anti-CD30L antibody or antigen binding fragment provided herein, including in Section 2 and this Section (Section 4.1) such as those of this paragraph, the antibody or antigen binding fragment thereof binds to CD30L with an association rate constant (kₒₙ) of at least 0.1×10⁶, at least 0.2×10⁶, at least 0.3×10⁶, at least 0.4×10⁶, at least 0.5×10⁶, at least 0.6×10⁶, at least 0.7×10⁶, at least 0.8×10⁶, at least 0.9×10⁶, at least 1.0×10⁶, at least 1.1×10⁶, at least 1.2×10⁶, at least 1.3×10⁶, at least 1.4×10⁶, at least 1.5×10⁶, at least 1.55×10⁶, at least 1.56×10⁶, at least 1.57×10⁶, at least 1.58×10⁶, at least 1.59×10⁶, at least 1.60×10⁶, at least 1.61×10⁶, at least 1.62×10⁶, at least 1.63×10⁶, at least 1.64×10⁶, at least 1.65×10⁶, at least 1.66×10⁶, at least 1.67×10⁶, at least 1.68×10⁶, at least 1.69×10⁶, at least 1.7×10⁶, at least 1.8×10⁶, at least 1.9×10⁶, at least 2.0×10⁶, at least 2.1×10⁶, at least 2.2×10⁶, at least 2.3×10⁶, at least 2.4×10⁶, at least 2.5×10⁶, at least 2.6×10⁶, at least 2.7×10⁶, at least 2.8×10⁶, at least 2.9×10⁶, at least 3.0×10⁶, or at least 3.1×10⁶M⁻¹ S⁻¹. In one embodiment of the anti-CD30L antibody or antigen binding fragment provided herein, including in Section 2 and this Section (Section 4.1) such as those of this paragraph, the antibody or antigen binding fragment thereof binds to CD30L with an association rate constant (kₒₙ) of about 0.1×10⁶, about 0.2×10⁶, about 0.3×10⁶, about 0.4×10⁶, about 0.5×10⁶, about 0.6×10⁶, about 0.7×10⁶, about 0.8×10⁶, about 0.9×10⁶, about 1.0×10⁶, about 1.1×10⁶, about 1.2×10⁶, about 1.3×10⁶, about 1.4×10⁶, about 1.5×10⁶, about 1.55×10⁶, about 1.56×10⁶, about 1.57×10⁶, about 1.58×10⁶, about 1.59×10⁶, about 1.60×10⁶, about 1.61×10⁶M⁻¹ S⁻¹, about 1.62×10⁶, about 1.63×10⁶, about 1.64×10⁶, about 1.65×10⁶, about 1.66×10⁶, about 1.67×10⁶, about 1.68×10⁶, about 1.69×10⁶, about 1.7×10⁶, about 1.8×10⁶, about 1.9×10⁶, about 2.0×10⁶, about 2.1×10⁶, about 2.2×10⁶, about 2.3×10⁶, about 2.4×10⁶, about 2.5×10⁶, about 2.6×10⁶, about 2.7×10⁶, about 2.8×10⁶, about 2.9×10⁶, about 3.0×10⁶, or about 3.1×10⁶ M⁻¹ S⁻¹. In one embodiment of the anti-CD30L antibody or antigen binding fragment provided herein, including in Section 2 and this Section (Section 4.1) such as those of this paragraph, the antibody or antigen binding fragment thereof binds to CD30L with an association rate constant (kₒₙ) of about 1.54×10⁶ to about 1.55×10⁶, about 1.53×10⁶ to about 1.56×10⁶, about 1.52×10⁶ to about 1.57×10⁶, about 1.53×10⁶ to about 1.58×10⁶, about 1.52×10⁶ to about 1.59×10⁶, about 1.51×10⁶ to about 1.60×10⁶, or about 1.5×10⁶ to about 1.61×10⁶M⁻¹ S⁻¹. In one embodiment of the anti-CD30L antibody or antigen binding fragment provided herein, including in Section 2 and this Section (Section 4.1) such as those of this paragraph, the antibody or antigen binding fragment thereof binds to CD30L with an association rate constant (kₒₙ) of about 2.5×10⁶ to about 3.8×10⁶, about 2.4×10⁶ to about 3.9×10⁶, about 2.45×10⁶ to about 3.89×10⁶, about 2.6×10⁶ to about 3.7×10⁶, about 2.7×10⁶ to about 3.6×10⁶, about 2.8×10⁶ to about 3.5×10⁶, about 2.9×10⁶ to about 3.4×10⁶, about 3.0×10⁶ to about 3.3×10⁶, or about 3.1×10⁶ to about 3.2×10⁶, M⁻¹ S⁻¹.

Furthermore, in one embodiment of the anti-CD30L antibody or antigen binding fragment provided herein, including in Section 2 and this Section (Section 4.1) such as those of this paragraph, the antibody or antigen binding fragment thereof binds to CD30L with a dissociation rate constant (k_{off}) of no more than 1.4×10⁻⁴, no more than 1.41×10⁻⁴, no more than 1.5×10⁻⁴, no more than 1.6×10⁻⁴, no more than 1.7×10⁻⁴, no more than 1.8×10⁻⁴, no more than 1.9×10⁻⁴, no more than 2.0×10⁻⁴, no more than 2.1×10⁻⁴, no more than 2.2×10⁻⁴, no more than 2.3×10⁻⁴, no more than 2.4×10⁻⁴, no more than 2.5×10⁻⁴, no more than 2.6×10⁻⁴, no more than 2.7×10⁻⁴, no more than 2.8×10⁻⁴, no more than 2.9×10⁻⁴, no more than 3.0×10⁻⁴, no more than 3.1×10⁻⁴, no more than 3.2×10⁻⁴, no more than 3.3×10⁻⁴, no more than 3.4×10⁻⁴, no more than 3.5×10⁻⁴, no more than 3.6×10⁻⁴, no more than 3.7×10⁻⁴, no more than 3.78×10⁻⁴, or no more than 3.8×10⁻⁴ S⁻¹. In one embodiment of the anti-CD30L antibody or antigen binding fragment provided herein, including in Section 2 and this Section (Section 4.1) such as those of this paragraph, the antibody or antigen binding fragment thereof binds to CD30L with a dissociation rate constant (k_{off}) of about 1.4×10⁻⁴, about 1.41×10⁻⁴, about 1.5×10⁻⁴, about 1.6×10⁻⁴, about 1.7×10⁻⁴, about 1.8×10⁻⁴, about 1.9×10⁻⁴, about 2.0×10⁻⁴, about 2.1×10⁻⁴, about 2.2×10⁻⁴, about 2.3×10⁻⁴, about 2.4×10⁻⁴, about 2.5×10⁻⁴, about 2.6×10⁻⁴, about 2.7×10⁻⁴, about 2.8×10⁻⁴, about 2.9×10⁻⁴, about 3.0×10⁻⁴, about 3.1×10⁻⁴, about 3.2×10⁻⁴, about 3.3×10⁻⁴, about 3.4×10⁻⁴, about 3.5×10⁻⁴ S⁻¹, about 3.6×10⁻⁴, about 3.7×10⁻⁴, about 3.78×10⁻⁴, or about 3.8×10⁻⁴ S⁻¹.

As described further above, the anti-CD30L antibody provided herein binds to specific epitope on CD30L. Such epitope maps differently on to the various CD30L isoforms. Accordingly, the disclosure further provides that the anti-CD30L antibody or antigen binding fragments provided herein binds differently to the CD30L isoforms. In one embodiment of the anti-CD30L antibody or antigen binding fragment provided herein, including in Section 2 and this Section (Section 4.1), the antibody or antigen binding fragment thereof binds isoform 1 of CD30L, and wherein the antibody or antigen binding fragment thereof does not bind to isoform 2 of CD30L. In one aspect, provided herein is an anti-CD30L antibody or antigen binding fragment thereof, wherein the antibody or antigen binding fragment thereof binds isoform 1 of CD30L, and wherein the antibody or antigen binding fragment thereof does not bind to isoform 2 of CD30L. In one embodiment of the anti-CD30L antibody or antigen binding fragment provided herein, including in Section 2 and this Section (Section 4.1) such as this paragraph, the isoform 1 of CD30L comprises the amino acid sequence set forth in SEQ ID NO: 34 and the isoform 2 of CD30L comprises the amino acid sequence set forth in SEQ ID NO: 35. In another embodiment of the anti-CD30L antibody or antigen binding fragment provided herein, including in Section 2 and this Section (Section 4.1) such as this paragraph, the isoform 1 of CD30L consists of the amino acid sequence set forth in SEQ ID NO: 34 and the isoform 2 of CD30L consists of the amino acid sequence set forth in SEQ ID NO: 35.

The disclosure demonstrates that the anti-CD30L antibody provided herein binds to human CD30L, cyno CD30L, or both human and cyno CD30L. In some embodiments of the anti-CD30L antibody or antigen binding fragment provided herein, including in Section 2 and this Section (Section 4.1), the antibody or antigen binding fragment thereof binds to human CD30L. In some embodiments of the anti-CD30L antibody or antigen binding fragment provided herein, including in Section 2 and this Section (Section 4.1), the antibody or antigen binding fragment thereof binds to cyno CD30L. In some embodiments of the anti-CD30L antibody or antigen binding fragment provided herein, including in Section 2 and this Section (Section 4.1), the antibody or antigen binding fragment thereof binds to both human and cyno CD30L. In some embodiments of the anti-CD30L antibody or antigen binding fragment provided herein, including in Section 2 and this Section (Section 4.1), the antibody or antigen binding fragment thereof specifically binds to human CD30L. In some embodiments of the anti-CD30L antibody or antigen binding fragment provided herein, including in Section 2 and this Section (Section 4.1), the antibody or antigen binding fragment thereof specifically binds to cyno CD30L. In some embodiments of the anti-CD30L antibody or antigen binding fragment provided herein, including in Section 2 and this Section (Section 4.1), the antibody or antigen binding fragment thereof specifically binds to both human and cyno CD30L.

In certain embodiments of the anti-CD30L antibody or antigen binding fragment provided herein, including in Section 2 and this Section (Section 4.1), the anti-CD30L antibody or antigen binding fragment thereof blocks both the soluble CD30L and the transmembrane CD30L. In one embodiment, the anti-CD30L antibody or antigen binding fragment thereof blocks the soluble CD30L. In another embodiment, the anti-CD30L antibody or antigen binding fragment thereof blocks the transmembrane CD30L. In some embodiments, the anti-CD30L antibody or antigen binding fragment provided herein, including in Section 2 and this Section (Section 4.1), the anti-CD30L antibody or antigen binding fragment thereof binds to both the soluble CD30L and the transmembrane CD30L. In one embodiment, the anti-CD30L antibody or antigen binding fragment thereof binds to the soluble CD30L. In another embodiment, the anti-CD30L antibody or antigen binding fragment thereof binds to the transmembrane CD30L.

In some embodiments of the anti-CD30L antibody or antigen binding fragment provided herein, including in Section 2 and this Section (Section 4.1), the anti-CD30L antibody or antigen binding fragment thereof comprises: (a) a CDR-H1 comprising the amino acid sequence set forth in any one of SEQ ID NOs: 100-139; (b) a CDR-H2 comprising the amino acid sequence set forth in any one of SEQ ID NOs: 140-179; (c) an immunoglobulin heavy chain CDR3 (CDR-H3) comprising the amino acid sequence set forth in any one of SEQ ID NOs: 180-219; (d) a CDR-L1 comprising the amino acid sequence set forth in any one of SEQ ID NOs: 300-339; (e) a CDR-L2 comprising the amino acid sequence set forth in any one of SEQ ID NOs: 340-379; and/or (f) a CDR-L3 comprising the amino acid sequence set forth in any one of SEQ ID NOs: 380-419.

In some embodiments, the antibody or antigen binding fragment thereof comprises: (a) a CDR-H1 comprising the amino acid sequence set forth in any one of SEQ ID NOs: 100-109; (b) a CDR-H2 comprising the amino acid sequence set forth in any one of SEQ ID NOs: 140-149; (c) an immunoglobulin heavy chain CDR3 (CDR-H3) comprising the amino acid sequence set forth in any one of SEQ ID NOs: 180-189; (d) a CDR-L1 comprising the amino acid sequence set forth in any one of SEQ ID NOs: 300-309; (e) a CDR-L2 comprising the amino acid sequence set forth in any one of SEQ ID NOs: 340-349; and/or (f) a CDR-L3 comprising the amino acid sequence set forth in any one of SEQ ID NOs: 380-389.

In certain embodiments, the antibody or antigen binding fragment thereof comprises: (a) a CDR-H1 comprising the amino acid sequence set forth in any one of SEQ ID NOs: 100-104; (b) a CDR-H2 comprising the amino acid sequence set forth in any one of SEQ ID NOs: 140-144; (c) an immunoglobulin heavy chain CDR3 (CDR-H3) comprising the amino acid sequence set forth in any one of SEQ ID NOs: 180-184; (d) a CDR-L1 comprising the amino acid sequence set forth in any one of SEQ ID NOs: 300-304; (e) a CDR-L2 comprising the amino acid sequence set forth in any one of SEQ ID NOs: 340-344; and/or (f) a CDR-L3 comprising the amino acid sequence set forth in any one of SEQ ID NOs: 380-384.

In certain embodiments, the antibody or antigen binding fragment thereof comprises: (a) a CDR-H1 comprising the amino acid sequence set forth in any one of SEQ ID NOs: 105-109; (b) a CDR-H2 comprising the amino acid sequence set forth in any one of SEQ ID NOs: 145-149; (c) an immunoglobulin heavy chain CDR3 (CDR-H3) comprising the amino acid sequence set forth in any one of SEQ ID NOs: 185-189; (d) a CDR-L1 comprising the amino acid sequence set forth in any one of SEQ ID NOs: 305-309; (e) a CDR-L2 comprising the amino acid sequence set forth in any one of SEQ ID NOs: 345-349; and/or (f) a CDR-L3 comprising the amino acid sequence set forth in any one of SEQ ID NOs: 385-389.

In some embodiments, the antibody or antigen binding fragment thereof comprises an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to any one of SEQ ID NOs: 1 and 2; and/or (b) the immunoglobulin variable region light chain comprises an amino acid sequence having at least about 90, 95, 97, 98, 99, or 100% sequence identity to any one of SEQ ID NOs: 3 and 4.

In certain embodiments, the immunoglobulin variable region heavy chain comprises an amino acid sequence having about 90% to about 100% sequence identity to any one of SEQ ID NOs: 1 and 2. In certain embodiments, the immunoglobulin variable region heavy chain comprises an amino acid sequence having at least about 90% sequence identity to any one of SEQ ID NOs: 1 and 2. In certain embodiments, the immunoglobulin variable region heavy chain comprises an amino acid sequence having at most about 100% sequence identity to any one of SEQ ID NOs: 1 and 2. In certain embodiments, the immunoglobulin variable region heavy chain comprises an amino acid sequence having about 90% to about 95% sequence identity to any one of SEQ ID NOs: 1 and 2, about 90% to about 96% sequence identity to any one of SEQ ID NOs: 1 and 2, about 90% to about 97% sequence identity to any one of SEQ ID NOs: 1 and 2, about 90% to about 98% sequence identity to any one of SEQ ID NOs: 1 and 2, about 90% to about 99% sequence identity to any one of SEQ ID NOs: 1 and 2, about 90% to about 100% sequence identity to any one of SEQ ID NOs: 1 and 2, about 95% to about 96% sequence identity to any one of SEQ ID NOs: 1 and 2, about 95% to about 97% sequence identity to any one of SEQ ID NOs: 1 and 2, about 95% to about 98% sequence identity to any one of SEQ ID NOs: 1 and 2, about 95% to about 99% sequence identity to any one of SEQ ID NOs: 1 and 2, about 95% to about 100% sequence identity to any one of SEQ ID NOs: 1 and 2, about 96% to about 97% sequence identity to any one of SEQ ID NOs: 1 and 2, about 96% to about 98% sequence identity to any one of SEQ ID NOs: 1 and 2, about 96% to about 99% sequence identity to any one of SEQ ID NOs: 1 and 2, about 96% to about 100% sequence identity to any one of SEQ ID NOs: 1 and 2, about 97% to about 98% sequence identity to any one of SEQ ID NOs: 1 and 2, about 97% to about 99% sequence identity to any one of SEQ ID NOs: 1 and 2, about 97% to about 100% sequence identity to any one of SEQ ID NOs: 1 and 2, about 98% to about 99% sequence identity to any one of SEQ ID NOs: 1 and 2, about 98% to about 100% sequence identity to any one of SEQ ID NOs: 1 and 2, or about 99% to about 100% sequence identity to any one of SEQ ID NOs: 1 and 2. In certain embodiments, the immunoglobulin variable region heavy chain comprises an amino acid sequence having about 90% sequence identity to any one of SEQ ID NOs: 1 and 2, about 95% sequence identity to any one of SEQ ID NOs: 1 and 2, about 97% sequence identity to any one of SEQ ID NOs: 1 and 2, about 98% sequence identity to any one of SEQ ID NOs: 1 and 2, about 99% sequence identity to any one of SEQ ID NOs: 1 and 2, or about 100% sequence identity to any one of SEQ ID NOs: 1 and 2.

In certain embodiments, the immunoglobulin variable region heavy chain comprises an amino acid sequence having about 90% to about 100% sequence identity to any one of SEQ ID NOs: 3 and 4. In certain embodiments, the immunoglobulin variable region heavy chain comprises an amino acid sequence having at least about 90% sequence identity to any one of SEQ ID NOs: 3 and 4. In certain embodiments, the immunoglobulin variable region heavy chain comprises an amino acid sequence having at most about 100% sequence identity to any one of SEQ ID NOs: 3 and 4. In certain embodiments, the immunoglobulin variable region heavy chain comprises an amino acid sequence having about 90% to about 95% sequence identity to any one of SEQ ID NOs: 3 and 4, about 90% to about 96% sequence identity to any one of SEQ ID NOs: 3 and 4, about 90% to about 97% sequence identity to any one of SEQ ID NOs: 3 and 4, about 90% to about 98% sequence identity to any one of SEQ ID NOs: 3 and 4, about 90% to about 99% sequence identity to any one of SEQ ID NOs: 3 and 4, about 90% to about 100% sequence identity to any one of SEQ ID NOs: 3 and 4, about 95% to about 96% sequence identity to any one of SEQ ID NOs: 3 and 4, about 95% to about 97% sequence identity to any one of SEQ ID NOs: 3 and 4, about 95% to about 98% sequence identity to any one of SEQ ID NOs: 3 and 4, about 95% to about 99% sequence identity to any one of SEQ ID NOs: 3 and 4, about 95% to about 100% sequence identity to any one of SEQ ID NOs: 3 and 4, about 96% to about 97% sequence identity to any one of SEQ ID NOs: 3 and 4, about 96% to about 98% sequence identity to any one of SEQ ID NOs: 3 and 4, about 96% to about 99% sequence identity to any one of SEQ ID NOs: 3 and 4, about 96% to about 100% sequence identity to any one of SEQ ID NOs: 3 and 4, about 97% to about 98% sequence identity to any one of SEQ ID NOs: 3 and 4, about 97% to about 99% sequence identity to any one of SEQ ID NOs: 3 and 4, about 97% to about 100% sequence identity to any one of SEQ ID NOs: 3 and 4, about 98% to about 99% sequence identity to any one of SEQ ID NOs: 3 and 4, about 98% to about 100% sequence identity to any one of SEQ ID NOs: 3 and 4, or about 99% to about 100% sequence identity to any one of SEQ ID NOs: 3 and 4. In certain embodiments, the immunoglobulin variable region heavy chain comprises an amino acid sequence having about 90% sequence identity to any one of SEQ ID NOs: 3 and 4, about 95% sequence identity to any one of SEQ ID NOs: 3 and 4, about 97% sequence identity to any one of SEQ ID NOs: 3 and 4, about 98% sequence identity to any one of SEQ ID NOs: 3 and 4, about 99% sequence identity to any one of SEQ ID NOs: 3 and 4, or about 100% sequence identity to any one of SEQ ID NOs: 3 and 4.

In some embodiments, the antibody or antigen binding fragment thereof comprises: (a) a CDR-H1 comprising the amino acid sequence set forth in any one of SEQ ID NOs: 110-119; (b) a CDR-H2 comprising the amino acid sequence set forth in any one of SEQ ID NOs: 150-159; (c) an immunoglobulin heavy chain CDR3 (CDR-H3) comprising the amino acid sequence set forth in any one of SEQ ID NOs: 190-199; (d) a CDR-L1 comprising the amino acid sequence set forth in any one of SEQ ID NOs: 310-319; (e) a CDR-L2 comprising the amino acid sequence set forth in any one of SEQ ID NOs: 350-359; and/or (f) a CDR-L3 comprising the amino acid sequence set forth in any one of SEQ ID NOs: 390-399.

In certain embodiments, the antibody or antigen binding fragment thereof comprises: (a) a CDR-H1 comprising the amino acid sequence set forth in any one of SEQ ID NOs: 110-114; (b) a CDR-H2 comprising the amino acid sequence set forth in any one of SEQ ID NOs: 150-154; (c) an immunoglobulin heavy chain CDR3 (CDR-H3) comprising the amino acid sequence set forth in any one of SEQ ID NOs: 190-194; (d) a CDR-L1 comprising the amino acid sequence set forth in any one of SEQ ID NOs: 310-314; (e) a CDR-L2 comprising the amino acid sequence set forth in any one of SEQ ID NOs: 350-354; and/or (f) a CDR-L3 comprising the amino acid sequence set forth in any one of SEQ ID NOs: 390-394.

In certain embodiments, the antibody or antigen binding fragment thereof comprises: (a) a CDR-H1 comprising the amino acid sequence set forth in any one of SEQ ID NOs: 115-119; (b) a CDR-H2 comprising the amino acid sequence set forth in any one of SEQ ID NOs: 155-159; (c) an immunoglobulin heavy chain CDR3 (CDR-H3) comprising the amino acid sequence set forth in any one of SEQ ID NOs: 195-199; (d) a CDR-L1 comprising the amino acid sequence set forth in any one of SEQ ID NOs: 315-319; (e) a CDR-L2 comprising the amino acid sequence set forth in any one of SEQ ID NOs: 355-359; and/or (f) a CDR-L3 comprising the amino acid sequence set forth in any one of SEQ ID NOs: 395-399.

In some embodiments, the antibody or antigen binding fragment thereof comprises an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to any one of SEQ ID NOs: 5 and 6; and/or (b) the immunoglobulin variable region light chain comprises an amino acid sequence having at least about 90, 95, 97, 98, 99, or 100% sequence identity to any one of SEQ ID NOs: 7 and 8.

In certain embodiments, the immunoglobulin variable region heavy chain comprises an amino acid sequence having about 90% sequence identity to any one of SEQ ID NOs: 5 and 6 to about 100% sequence identity to any one of SEQ ID NOs: 5 and 6. In certain embodiments, the immunoglobulin variable region heavy chain comprises an amino acid sequence having at least about 90% sequence identity to any one of SEQ ID NOs: 5 and 6. In certain embodiments, the immunoglobulin variable region heavy chain comprises an amino acid sequence having at most about 100% sequence identity to any one of SEQ ID NOs: 5 and 6. In certain embodiments, the immunoglobulin variable region heavy chain comprises an amino acid sequence having about 90% sequence identity to any one of SEQ ID NOs: 5 and 6 to about 95% sequence identity to any one of SEQ ID NOs: 5 and 6, about 90% sequence identity to any one of SEQ ID NOs: 5 and 6 to about 97% sequence identity to any one of SEQ ID NOs: 5 and 6, about 90% sequence identity to any one of SEQ ID NOs: 5 and 6 to about 98% sequence identity to any one of SEQ ID NOs: 5 and 6, about 90% sequence identity to any one of SEQ ID NOs: 5 and 6 to about 99% sequence identity to any one of SEQ ID NOs: 5 and 6, about 90% sequence identity to any one of SEQ ID NOs: 5 and 6 to about 100% sequence identity to any one of SEQ ID NOs: 5 and 6, about 95% sequence identity to any one of SEQ ID NOs: 5 and 6 to about 97% sequence identity to any one of SEQ ID NOs: 5 and 6, about 95% sequence identity to any one of SEQ ID NOs: 5 and 6 to about 98% sequence identity to any one of SEQ ID NOs: 5 and 6, about 95% sequence identity to any one of SEQ ID NOs: 5 and 6 to about 99% sequence identity to any one of SEQ ID NOs: 5 and 6, about 95% sequence identity to any one of SEQ ID NOs: 5 and 6 to about 100% sequence identity to any one of SEQ ID NOs: 5 and 6, about 97% sequence identity to any one of SEQ ID NOs: 5 and 6 to about 98% sequence identity to any one of SEQ ID NOs: 5 and 6, about 97% sequence identity to any one of SEQ ID NOs: 5 and 6 to about 99% sequence identity to any one of SEQ ID NOs: 5 and 6, about 97% sequence identity to any one of SEQ ID NOs: 5 and 6 to about 100% sequence identity to any one of SEQ ID NOs: 5 and 6, about 98% sequence identity to any one of SEQ ID NOs: 5 and 6 to about 99% sequence identity to any one of SEQ ID NOs: 5 and 6, about 98% sequence identity to any one of SEQ ID NOs: 5 and 6 to about 100% sequence identity to any one of SEQ ID NOs: 5 and 6, or about 99% sequence identity to any one of SEQ ID NOs: 5 and 6 to about 100% sequence identity to any one of SEQ ID NOs: 5 and 6. In certain embodiments, the immunoglobulin variable region heavy chain comprises an amino acid sequence having about 90% sequence identity to any one of SEQ ID NOs: 5 and 6, about 95% sequence identity to any one of SEQ ID NOs: 5 and 6, about 97% sequence identity to any one of SEQ ID NOs: 5 and 6, about 98% sequence identity to any one of SEQ ID NOs: 5 and 6, about 99% sequence identity to any one of SEQ ID NOs: 5 and 6, or about 100% sequence identity to any one of SEQ ID NOs: 5 and 6.

In certain embodiments, the immunoglobulin variable region light chain comprises an amino acid sequence having about 90% sequence identity to any one of SEQ ID NOs: 7 and 8 to about 100% sequence identity to any one of SEQ ID NOs: 7 and 8. In certain embodiments, the immunoglobulin variable region light chain comprises an amino acid sequence having at least about 90% sequence identity to any one of SEQ ID NOs: 7 and 8. In certain embodiments, the immunoglobulin variable region light chain comprises an amino acid sequence having at most about 100% sequence identity to any one of SEQ ID NOs: 7 and 8. In certain embodiments, the immunoglobulin variable region light chain comprises an amino acid sequence having about 90% sequence identity to any one of SEQ ID NOs: 7 and 8 to about 95% sequence identity to any one of SEQ ID NOs: 7 and 8, about 90% sequence identity to any one of SEQ ID NOs: 7 and 8 to about 97% sequence identity to any one of SEQ ID NOs: 7 and 8, about 90% sequence identity to any one of SEQ ID NOs: 7 and 8 to about 98% sequence identity to any one of SEQ ID NOs: 7 and 8, about 90% sequence identity to any one of SEQ ID NOs: 7 and 8 to about 99% sequence identity to any one of SEQ ID NOs: 7 and 8, about 90% sequence identity to any one of SEQ ID NOs: 7 and 8 to about 100% sequence identity to any one of SEQ ID NOs: 7 and 8, about 95% sequence identity to any one of SEQ ID NOs: 7 and 8 to about 97% sequence identity to any one of SEQ ID NOs: 7 and 8, about 95% sequence identity to any one of SEQ ID NOs: 7 and 8 to about 98% sequence identity to any one of SEQ ID NOs: 7 and 8, about 95% sequence identity to any one of SEQ ID NOs: 7 and 8 to about 99% sequence identity to any one of SEQ ID NOs: 7 and 8, about 95% sequence identity to any one of SEQ ID NOs: 7 and 8 to about 100% sequence identity to any one of SEQ ID NOs: 7 and 8, about 97% sequence identity to any one of SEQ ID NOs: 7 and 8 to about 98% sequence identity to any one of SEQ ID NOs: 7 and 8, about 97% sequence identity to any one of SEQ ID NOs: 7 and 8 to about 99% sequence identity to any one of SEQ ID NOs: 7 and 8, about 97% sequence identity to any one of SEQ ID NOs: 7 and 8 to about 100% sequence identity to any one of SEQ ID NOs: 7 and 8, about 98% sequence identity to any one of SEQ ID NOs: 7 and 8 to about 99% sequence identity to any one of SEQ ID NOs: 7 and 8, about 98% sequence identity to any one of SEQ ID NOs: 7 and 8 to about 100% sequence identity to any one of SEQ ID NOs: 7 and 8, or about 99% sequence identity to any one of SEQ ID NOs: 7 and 8 to about 100% sequence identity to any one of SEQ ID NOs: 7 and 8. In certain embodiments, the immunoglobulin variable region light chain comprises an amino acid sequence having about 90% sequence identity to any one of SEQ ID NOs: 7 and 8, about 95% sequence identity to any one of SEQ ID NOs: 7 and 8, about 97% sequence identity to any one of SEQ ID NOs: 7 and 8, about 98% sequence identity to any one of SEQ ID NOs: 7 and 8, about 99% sequence identity to any one of SEQ ID NOs: 7 and 8, or about 100% sequence identity to any one of SEQ ID NOs: 7 and 8.

In some embodiments, the antibody or antigen binding fragment thereof comprises: (a) a CDR-H1 comprising the amino acid sequence set forth in any one of SEQ ID NOs: 120-129; (b) a CDR-H2 comprising the amino acid sequence set forth in any one of SEQ ID NOs: 160-169; (c) an immunoglobulin heavy chain CDR3 (CDR-H3) comprising the amino acid sequence set forth in any one of SEQ ID NOs: 200-209; (d) a CDR-L1 comprising the amino acid sequence set forth in any one of SEQ ID NOs: 320-329; (e) a CDR-L2 comprising the amino acid sequence set forth in any one of SEQ ID NOs: 360-369; and/or (f) a CDR-L3 comprising the amino acid sequence set forth in any one of SEQ ID NOs: 400-409.

In certain embodiments, the antibody or antigen binding fragment thereof comprises: (a) a CDR-H1 comprising the amino acid sequence set forth in any one of SEQ ID NOs: 120-124; (b) a CDR-H2 comprising the amino acid sequence set forth in any one of SEQ ID NOs: 160-164; (c) an immunoglobulin heavy chain CDR3 (CDR-H3) comprising the amino acid sequence set forth in any one of SEQ ID NOs: 200-204; (d) a CDR-L1 comprising the amino acid sequence set forth in any one of SEQ ID NOs: 320-324; (e) a CDR-L2 comprising the amino acid sequence set forth in any one of SEQ ID NOs: 360-364; and/or (f) a CDR-L3 comprising the amino acid sequence set forth in any one of SEQ ID NOs: 400-404.

In certain embodiments, the antibody or antigen binding fragment thereof comprises: (a) a CDR-H1 comprising the amino acid sequence set forth in any one of SEQ ID NOs: 125-129; (b) a CDR-H2 comprising the amino acid sequence set forth in any one of SEQ ID NOs: 165-169; (c) an immunoglobulin heavy chain CDR3 (CDR-H3) comprising the amino acid sequence set forth in any one of SEQ ID NOs: 205-209; (d) a CDR-L1 comprising the amino acid sequence set forth in any one of SEQ ID NOs: 325-329; (e) a CDR-L2 comprising the amino acid sequence set forth in any one of SEQ ID NOs: 365-369; and/or (f) a CDR-L3 comprising the amino acid sequence set forth in any one of SEQ ID NOs: 405-409.

In some embodiments, the antibody or antigen binding fragment thereof comprises an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to any one of SEQ ID NOs: 9 and 10; and/or (b) the immunoglobulin variable region light chain comprises an amino acid sequence having at least about 90, 95, 97, 98, 99, or 100% sequence identity to any one of SEQ ID NOs: 11 and 12.

In certain embodiments, the immunoglobulin variable region heavy chain comprises an amino acid sequence having about 90% sequence identity to any one of SEQ ID NOs: 9 and 10 to about 100% sequence identity to any one of SEQ ID NOs: 9 and 10. In certain embodiments, the immunoglobulin variable region heavy chain comprises an amino acid sequence having at least about 90% sequence identity to any one of SEQ ID NOs: 9 and 10. In certain embodiments, the immunoglobulin variable region heavy chain comprises an amino acid sequence having at most about 100% sequence identity to any one of SEQ ID NOs: 9 and 10. In certain embodiments, the immunoglobulin variable region heavy chain comprises an amino acid sequence having about 90% sequence identity to any one of SEQ ID NOs: 9 and 10 to about 95% sequence identity to any one of SEQ ID NOs: 9 and 10, about 90% sequence identity to any one of SEQ ID NOs: 9 and 10 to about 97% sequence identity to any one of SEQ ID NOs: 9 and 10, about 90% sequence identity to any one of SEQ ID NOs: 9 and 10 to about 98% sequence identity to any one of SEQ ID NOs: 9 and 10, about 90% sequence identity to any one of SEQ ID NOs: 9 and 10 to about 99% sequence identity to any one of SEQ ID NOs: 9 and 10, about 90% sequence identity to any one of SEQ ID NOs: 9 and 10 to about 100% sequence identity to any one of SEQ ID NOs: 9 and 10, about 95% sequence identity to any one of SEQ ID NOs: 9 and 10 to about 97% sequence identity to any one of SEQ ID NOs: 9 and 10, about 95% sequence identity to any one of SEQ ID NOs: 9 and 10 to about 98% sequence identity to any one of SEQ ID NOs: 9 and 10, about 95% sequence identity to any one of SEQ ID NOs: 9 and 10 to about 99% sequence identity to any one of SEQ ID NOs: 9 and 10, about 95% sequence identity to any one of SEQ ID NOs: 9 and 10 to about 100% sequence identity to any one of SEQ ID NOs: 9 and 10, about 97% sequence identity to any one of SEQ ID NOs: 9 and 10 to about 98% sequence identity to any one of SEQ ID NOs: 9 and 10, about 97% sequence identity to any one of SEQ ID NOs: 9 and 10 to about 99% sequence identity to any one of SEQ ID NOs: 9 and 10, about 97% sequence identity to any one of SEQ ID NOs: 9 and 10 to about 100% sequence identity to any one of SEQ ID NOs: 9 and 10, about 98% sequence identity to any one of SEQ ID NOs: 9 and 10 to about 99% sequence identity to any one of SEQ ID NOs: 9 and 10, about 98% sequence identity to any one of SEQ ID NOs: 9 and 10 to about 100% sequence identity to any one of SEQ ID NOs: 9 and 10, or about 99% sequence identity to any one of SEQ ID NOs: 9 and 10 to about 100% sequence identity to any one of SEQ ID NOs: 9 and 10. In certain embodiments, the immunoglobulin variable region heavy chain comprises an amino acid sequence having about 90% sequence identity to any one of SEQ ID NOs: 9 and 10, about 95% sequence identity to any one of SEQ ID NOs: 9 and 10, about 97% sequence identity to any one of SEQ ID NOs: 9 and 10, about 98% sequence identity to any one of SEQ ID NOs: 9 and 10, about 99% sequence identity to any one of SEQ ID NOs: 9 and 10, or about 100% sequence identity to any one of SEQ ID NOs: 9 and 10.

In certain embodiments, the immunoglobulin variable region light chain comprises an amino acid sequence having about 90% sequence identity to any one of SEQ ID NOs: 11 and 12 to about 100% sequence identity to any one of SEQ ID NOs: 11 and 12. In certain embodiments, the immunoglobulin variable region light chain comprises an amino acid sequence having at least about 90% sequence identity to any one of SEQ ID NOs: 11 and 12. In certain embodiments, the immunoglobulin variable region light chain comprises an amino acid sequence having at most about 100% sequence identity to any one of SEQ ID NOs: 11 and 12. In certain embodiments, the immunoglobulin variable region light chain comprises an amino acid sequence having about 90% sequence identity to any one of SEQ ID NOs: 11 and 12 to about 95% sequence identity to any one of SEQ ID NOs: 11 and 12, about 90% sequence identity to any one of SEQ ID NOs: 11 and 12 to about 97% sequence identity to any one of SEQ ID NOs: 11 and 12, about 90% sequence identity to any one of SEQ ID NOs: 11 and 12 to about 98% sequence identity to any one of SEQ ID NOs: 11 and 12, about 90% sequence identity to any one of SEQ ID NOs: 11 and 12 to about 99% sequence identity to any one of SEQ ID NOs: 11 and 12, about 90% sequence identity to any one of SEQ ID NOs: 11 and 12 to about 100% sequence identity to any one of SEQ ID NOs: 11 and 12, about 95% sequence identity to any one of SEQ ID NOs: 11 and 12 to about 97% sequence identity to any one of SEQ ID NOs: 11 and 12, about 95% sequence identity to any one of SEQ ID NOs: 11 and 12 to about 98% sequence identity to any one of SEQ ID NOs: 11 and 12, about 95% sequence identity to any one of SEQ ID NOs: 11 and 12 to about 99% sequence identity to any one of SEQ ID NOs: 11 and 12, about 95% sequence identity to any one of SEQ ID NOs: 11 and 12 to about 100% sequence identity to any one of SEQ ID NOs: 11 and 12, about 97% sequence identity to any one of SEQ ID NOs: 11 and 12 to about 98% sequence identity to any one of SEQ ID NOs: 11 and 12, about 97% sequence identity to any one of SEQ ID NOs: 11 and 12 to about 99% sequence identity to any one of SEQ ID NOs: 11 and 12, about 97% sequence identity to any one of SEQ ID NOs: 11 and 12 to about 100% sequence identity to any one of SEQ ID NOs: 11 and 12, about 98% sequence identity to any one of SEQ ID NOs: 11 and 12 to about 99% sequence identity to any one of SEQ ID NOs: 11 and 12, about 98% sequence identity to any one of SEQ ID NOs: 11 and 12 to about 100% sequence identity to any one of SEQ ID NOs: 11 and 12, or about 99% sequence identity to any one of SEQ ID NOs: 11 and 12 to about 100% sequence identity to any one of SEQ ID NOs: 11 and 12. In certain embodiments, the immunoglobulin variable region light chain comprises an amino acid sequence having about 90% sequence identity to any one of SEQ ID NOs: 11 and 12, about 95% sequence identity to any one of SEQ ID NOs: 11 and 12, about 97% sequence identity to any one of SEQ ID NOs: 11 and 12, about 98% sequence identity to any one of SEQ ID NOs: 11 and 12, about 99% sequence identity to any one of SEQ ID NOs: 11 and 12, or about 100% sequence identity to any one of SEQ ID NOs: 11 and 12.

In some embodiments, the antibody or antigen binding fragment thereof comprises: (a) a CDR-H1 comprising the amino acid sequence set forth in any one of SEQ ID NOs: 130-139; (b) a CDR-H2 comprising the amino acid sequence set forth in any one of SEQ ID NOs: 170-179; (c) an immunoglobulin heavy chain CDR3 (CDR-H3) comprising the amino acid sequence set forth in any one of SEQ ID NOs: 210-219; (d) a CDR-L1 comprising the amino acid sequence set forth in any one of SEQ ID NOs: 330-339; (e) a CDR-L2 comprising the amino acid sequence set forth in any one of SEQ ID NOs: 370-379; and/or (f) a CDR-L3 comprising the amino acid sequence set forth in any one of SEQ ID NOs: 410-419.

In certain embodiments, the antibody or antigen binding fragment thereof comprises: (a) a CDR-H1 comprising the amino acid sequence set forth in any one of SEQ ID NOs: 130-134; (b) a CDR-H2 comprising the amino acid sequence set forth in any one of SEQ ID NOs: 170-174; (c) an immunoglobulin heavy chain CDR3 (CDR-H3) comprising the amino acid sequence set forth in any one of SEQ ID NOs: 210-214; (d) a CDR-L1 comprising the amino acid sequence set forth in any one of SEQ ID NOs: 330-334; (e) a CDR-L2 comprising the amino acid sequence set forth in any one of SEQ ID NOs: 370-374; and/or (f) a CDR-L3 comprising the amino acid sequence set forth in any one of SEQ ID NOs: 410-414.

In certain embodiments, the antibody or antigen binding fragment thereof comprises: (a) a CDR-H1 comprising the amino acid sequence set forth in any one of SEQ ID NOs: 135-139; (b) a CDR-H2 comprising the amino acid sequence set forth in any one of SEQ ID NOs: 175-179; (c) an immunoglobulin heavy chain CDR3 (CDR-H3) comprising the amino acid sequence set forth in any one of SEQ ID NOs: 215-219; (d) a CDR-L1 comprising the amino acid sequence set forth in any one of SEQ ID NOs: 335-339; (e) a CDR-L2 comprising the amino acid sequence set forth in any one of SEQ ID NOs: 375-379; and/or (f) a CDR-L3 comprising the amino acid sequence set forth in any one of SEQ ID NOs: 415-419.

In some embodiments, the antibody or antigen binding fragment thereof comprises an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to any one of SEQ ID NOs: 13 and 14; and/or (b) the immunoglobulin variable region light chain comprises an amino acid sequence having at least about 90, 95, 97, 98, 99, or 100% sequence identity to any one of SEQ ID NOs: 15 and 16.

In certain embodiments, the immunoglobulin variable region heavy chain comprises an amino acid sequence having about 90% sequence identity to any one of SEQ ID NOs: 13 and 14 to about 100% sequence identity to any one of SEQ ID NOs: 13 and 14. In certain embodiments, the immunoglobulin variable region heavy chain comprises an amino acid sequence having at least about 90% sequence identity to any one of SEQ ID NOs: 13 and 14. In certain embodiments, the immunoglobulin variable region heavy chain comprises an amino acid sequence having at most about 100% sequence identity to any one of SEQ ID NOs: 13 and 14. In certain embodiments, the immunoglobulin variable region heavy chain comprises an amino acid sequence having about 90% sequence identity to any one of SEQ ID NOs: 13 and 14 to about 95% sequence identity to any one of SEQ ID NOs: 13 and 14, about 90% sequence identity to any one of SEQ ID NOs: 13 and 14 to about 97% sequence identity to any one of SEQ ID NOs: 13 and 14, about 90% sequence identity to any one of SEQ ID NOs: 13 and 14 to about 98% sequence identity to any one of SEQ ID NOs: 13 and 14, about 90% sequence identity to any one of SEQ ID NOs: 13 and 14 to about 99% sequence identity to any one of SEQ ID NOs: 13 and 14, about 90% sequence identity to any one of SEQ ID NOs: 13 and 14 to about 100% sequence identity to any one of SEQ ID NOs: 13 and 14, about 95% sequence identity to any one of SEQ ID NOs: 13 and 14 to about 97% sequence identity to any one of SEQ ID NOs: 13 and 14, about 95% sequence identity to any one of SEQ ID NOs: 13 and 14 to about 98% sequence identity to any one of SEQ ID NOs: 13 and 14, about 95% sequence identity to any one of SEQ ID NOs: 13 and 14 to about 99% sequence identity to any one of SEQ ID NOs: 13 and 14, about 95% sequence identity to any one of SEQ ID NOs: 13 and 14 to about 100% sequence identity to any one of SEQ ID NOs: 13 and 14, about 97% sequence identity to any one of SEQ ID NOs: 13 and 14 to about 98% sequence identity to any one of SEQ ID NOs: 13 and 14, about 97% sequence identity to any one of SEQ ID NOs: 13 and 14 to about 99% sequence identity to any one of SEQ ID NOs: 13 and 14, about 97% sequence identity to any one of SEQ ID NOs: 13 and 14 to about 100% sequence identity to any one of SEQ ID NOs: 13 and 14, about 98% sequence identity to any one of SEQ ID NOs: 13 and 14 to about 99% sequence identity to any one of SEQ ID NOs: 13 and 14, about 98% sequence identity to any one of SEQ ID NOs: 13 and 14 to about 100% sequence identity to any one of SEQ ID NOs: 13 and 14, or about 99% sequence identity to any one of SEQ ID NOs: 13 and 14 to about 100% sequence identity to any one of SEQ ID NOs: 13 and 14. In certain embodiments, the immunoglobulin variable region heavy chain comprises an amino acid sequence having about 90% sequence identity to any one of SEQ ID NOs: 13 and 14, about 95% sequence identity to any one of SEQ ID NOs: 13 and 14, about 97% sequence identity to any one of SEQ ID NOs: 13 and 14, about 98% sequence identity to any one of SEQ ID NOs: 13 and 14, about 99% sequence identity to any one of SEQ ID NOs: 13 and 14, or about 100% sequence identity to any one of SEQ ID NOs: 13 and 14.

In certain embodiments, the immunoglobulin variable region light chain comprises an amino acid sequence having about 90% sequence identity to any one of SEQ ID NOs: 15 and 16 to about 100% sequence identity to any one of SEQ ID NOs: 15 and 16. In certain embodiments, the immunoglobulin variable region light chain comprises an amino acid sequence having at least about 90% sequence identity to any one of SEQ ID NOs: 15 and 16. In certain embodiments, the immunoglobulin variable region light chain comprises an amino acid sequence having at most about 100% sequence identity to any one of SEQ ID NOs: 15 and 16. In certain embodiments, the immunoglobulin variable region light chain comprises an amino acid sequence having about 90% sequence identity to any one of SEQ ID NOs: 15 and 16 to about 95% sequence identity to any one of SEQ ID NOs: 15 and 16, about 90% sequence identity to any one of SEQ ID NOs: 15 and 16 to about 97% sequence identity to any one of SEQ ID NOs: 15 and 16, about 90% sequence identity to any one of SEQ ID NOs: 15 and 16 to about 98% sequence identity to any one of SEQ ID NOs: 15 and 16, about 90% sequence identity to any one of SEQ ID NOs: 15 and 16 to about 99% sequence identity to any one of SEQ ID NOs: 15 and 16, about 90% sequence identity to any one of SEQ ID NOs: 15 and 16 to about 100% sequence identity to any one of SEQ ID NOs: 15 and 16, about 95% sequence identity to any one of SEQ ID NOs: 15 and 16 to about 97% sequence identity to any one of SEQ ID NOs: 15 and 16, about 95% sequence identity to any one of SEQ ID NOs: 15 and 16 to about 98% sequence identity to any one of SEQ ID NOs: 15 and 16, about 95% sequence identity to any one of SEQ ID NOs: 15 and 16 to about 99% sequence identity to any one of SEQ ID NOs: 15 and 16, about 95% sequence identity to any one of SEQ ID NOs: 15 and 16 to about 100% sequence identity to any one of SEQ ID NOs: 15 and 16, about 97% sequence identity to any one of SEQ ID NOs: 15 and 16 to about 98% sequence identity to any one of SEQ ID NOs: 15 and 16, about 97% sequence identity to any one of SEQ ID NOs: 15 and 16 to about 99% sequence identity to any one of SEQ ID NOs: 15 and 16, about 97% sequence identity to any one of SEQ ID NOs: 15 and 16 to about 100% sequence identity to any one of SEQ ID NOs: 15 and 16, about 98% sequence identity to any one of SEQ ID NOs: 15 and 16 to about 99% sequence identity to any one of SEQ ID NOs: 15 and 16, about 98% sequence identity to any one of SEQ ID NOs: 15 and 16 to about 100% sequence identity to any one of SEQ ID NOs: 15 and 16, or about 99% sequence identity to any one of SEQ ID NOs: 15 and 16 to about 100% sequence identity to any one of SEQ ID NOs: 15 and 16. In certain embodiments, the immunoglobulin variable region light chain comprises an amino acid sequence having about 90% sequence identity to any one of SEQ ID NOs: 15 and 16, about 95% sequence identity to any one of SEQ ID NOs: 15 and 16, about 97% sequence identity to any one of SEQ ID NOs: 15 and 16, about 98% sequence identity to any one of SEQ ID NOs: 15 and 16, about 99% sequence identity to any one of SEQ ID NOS: 15 and 16, or about 100% sequence identity to any one of SEQ ID NOs: 15 and 16.

In some embodiments of the anti-CD30L antibody or antigen binding fragment provided herein, including in Section 2 and this Section (Section 4.1), the antibody or antigen binding fragment thereof is recombinant antibody or antigen binding fragment thereof.

In certain embodiments of the anti-CD30L antibody or antigen binding fragment provided herein, including in Section 2 and this Section (Section 4.1), the antibody or antigen binding fragment thereof is isolated antibody or antigen binding fragment thereof.

In one additional aspect, provided herein is an antibody or antigen-binding fragment thereof that binds to an epitope of human CD30L recognized by an anti-CD30L antibody or antigen binding fragment provided herein, including in Section 2 and this Section (Section 4.1 such as the preceding paragraphs). Epitope recognized by the anti-CD30L antibodies or antigen binding fragments provided herein can be determined by means described in Sections 4.1 and/or Section 5.

In another aspect, provided herein is an antibody or antigen-binding fragment thereof that competes for the binding to human CD30L with an anti-CD30L antibody or antigen binding fragment provided herein, including in Section 2 and this Section (Section 4.1 such as the preceding paragraphs). Competition of binding to CD30L between the anti-CD30L antibodies (or antigen binding fragments) can be determined by means described in Sections 4.1 and/or Section 5.

In certain embodiments of the anti-CD30L antibody or antigen binding fragment provided herein, including in Section 2 and this Section (Section 4.1), the CD30L is a human CD30L. In some embodiments of the anti-CD30L antibody or antigen binding fragment provided herein, including in Section 2 and this Section (Section 4.1), the CD30L is a cyno CD30L. In some embodiments of the anti-CD30L antibody or antigen binding fragment provided herein, including in Section 2 and this Section (Section 4.1), the CD30L is both a human CD30L and a cyno CD30L. Other specific embodiments of anti-CD30L antibodies or antigen binding fragments that can bind to murine CD30L have also been specifically described above.

### 4.2 Germline Reversions

In some instances, germline reversions (for example, mutations of an antibody sequence back to the germline sequence) are useful for improving the safety, pharmacodynamic and/or pharmacokinetic properties of the anti-CD30L antibodies. For example, in certain instances, germline reversion mutations reduce immunogenicity of an anti-CD30L antibody while maintaining the ability to bind CD30L with high affinity and specificity.

Generally, a germline antibody gene or germline antibody gene segment refers to a sequence encoding an immunoglobulin present in the genome of an organism, wherein the genome is not or has not been subjected to mutational processes such a somatic hypermutation (for example, has not experienced a maturation process that leads to genetic rearrangements and mutations that express specific immunoglobulins). Thus, in some instances, the heavy chain germline gene refers to a germline antibody gene or gene fragment encoding an immunoglobulin heavy chain, which includes a V gene (variable), a D gene (diversity), and a J gene (joining), and in certain instances, the C gene (constant). Similarly, in some instances, the light chain germline gene refers to a germline antibody gene or gene fragment encoding an immunoglobulin light chain, which includes a V gene (variable), a J gene (joining), and in some instances, the C gene (constant). In some instances, the amino acid sequence encoded by the germline antibody gene or the germline antibody gene fragment is also referred to as a germline sequence. In certain instances, an amino acids sequence mutation back to a germline amino acid sequence refers to or identifies an amino acid mutation (for example, in a matured antibody amino acid sequence) to the amino acid of the germline amino acid (for example, in the germline antibody sequence). In certain instances, identifying a germline antibody gene or germline antibody gene fragments and their corresponding germline sequences can be obtained or queried from a professional database (for example, IMGT, UNSWIg, NCBI or VBASE2).

Accordingly, in some embodiments, the anti-CD30L comprises one or more amino acid sequence mutations back to a germline amino acid sequence. In certain embodiments, the recombinant antibody or antigen binding fragment thereof comprises a heavy chain variable region and/or light chain variable region comprising about 1 mutation back to a germline sequence to about 20 mutations back to a germline sequence. In certain embodiments, the recombinant antibody or antigen binding fragment thereof comprises a heavy chain variable region and/or light chain variable region comprising at least about 1 mutation back to a germline sequence. In certain embodiments, the recombinant antibody or antigen binding fragment thereof comprises a heavy chain variable region and/or light chain variable region comprising at most about 20 mutations back to a germline sequence. In certain embodiments, the recombinant antibody or antigen binding fragment thereof comprises a heavy chain variable region and/or light chain variable region comprising about 1 mutation back to a germline sequence to about 2 mutations back to a germline sequence, about 1 mutation back to a germline sequence to about 3 mutations back to a germline sequence, about 1 mutation back to a germline sequence to about 4 mutations back to a germline sequence, about 1 mutation back to a germline sequence to about 5 mutations back to a germline sequence, about 1 mutation back to a germline sequence to about 10 mutations back to a germline sequence, about 1 mutation back to a germline sequence to about 15 mutations back to a germline sequence, about 1 mutation back to a germline sequence to about 20 mutations back to a germline sequence, about 2 mutations back to a germline sequence to about 3 mutations back to a germline sequence, about 2 mutations back to a germline sequence to about 4 mutations back to a germline sequence, about 2 mutations back to a germline sequence to about 5 mutations back to a germline sequence, about 2 mutations back to a germline sequence to about 10 mutations back to a germline sequence, about 2 mutations back to a germline sequence to about 15 mutations back to a germline sequence, about 2 mutations back to a germline sequence to about 20 mutations back to a germline sequence, about 3 mutations back to a germline sequence to about 4 mutations back to a germline sequence, about 3 mutations back to a germline sequence to about 5 mutations back to a germline sequence, about 3 mutations back to a germline sequence to about 10 mutations back to a germline sequence, about 3 mutations back to a germline sequence to about 15 mutations back to a germline sequence, about 3 mutations back to a germline sequence to about 20 mutations back to a germline sequence, about 4 mutations back to a germline sequence to about 5 mutations back to a germline sequence, about 4 mutations back to a germline sequence to about 10 mutations back to a germline sequence, about 4 mutations back to a germline sequence to about 15 mutations back to a germline sequence, about 4 mutations back to a germline sequence to about 20 mutations back to a germline sequence, about 5 mutations back to a germline sequence to about 10 mutations back to a germline sequence, about 5 mutations back to a germline sequence to about 15 mutations back to a germline sequence, about 5 mutations back to a germline sequence to about 20 mutations back to a germline sequence, about 10 mutations back to a germline sequence to about 15 mutations back to a germline sequence, about 10 mutations back to a germline sequence to about 20 mutations back to a germline sequence, or about 15 mutations back to a germline sequence to about 20 mutations back to a germline sequence. In certain embodiments, the recombinant antibody or antigen binding fragment thereof comprises a heavy chain variable region and/or light chain variable region comprising about 1 mutation back to a germline sequence, about 2 mutations back to a germline sequence, about 3 mutations back to a germline sequence, about 4 mutations back to a germline sequence, about 5 mutations back to a germline sequence, about 10 mutations back to a germline sequence, about 15 mutations back to a germline sequence, or about 20 mutations back to a germline sequence.

In some embodiments, a recombinant antibody or antigen binding fragment thereof comprising an immunoglobulin variable region heavy chain and an immunoglobulin variable region light chain as set forth in SEQ ID NOs: 1 and 3 comprises one or more amino acid sequence mutations back to a germline amino acid sequence, thereby yielding a germline reverted or germlined anti-CD30L antibody. In certain embodiments, the germline reverted or germlined anti-CD30L antibody comprises the amino acid sequence as set forth in SEQ ID NOs: 2 and 4, or an amino acid sequence at least 90, 95, 95, 96, 97, 98, or 99 percent identical to the amino acid sequence as set forth in SEQ ID NOs: 2 and 4.

In some embodiments, a recombinant antibody or antigen binding fragment thereof comprising an immunoglobulin variable region heavy chain and an immunoglobulin variable region light chain as set forth in SEQ ID NOs: 5 and 7 comprises one or more amino acid sequence mutations back to a germline amino acid sequence, thereby yielding a germline reverted or germlined anti-CD30L antibody. In certain embodiments, the germline reverted or germlined anti-CD30L antibody comprises the amino acid sequence as set forth in SEQ ID NOs: 6 and 8, or an amino acid sequence at least 90, 95, 95, 96, 97, 98, or 99 percent identical to the amino acid sequence as set forth in SEQ ID NOs: 6 and 8.

In some embodiments, a recombinant antibody or antigen binding fragment thereof comprising an immunoglobulin variable region heavy chain and an immunoglobulin variable region light chain as set forth in SEQ ID NOs: 9 and 11 comprises one or more amino acid sequence mutations back to a germline amino acid sequence, thereby yielding a germline reverted or germlined anti-CD30L antibody. In certain embodiments, the germline reverted or germlined anti-CD30L antibody comprises the amino acid sequence as set forth in SEQ ID NOs: 10 and 12, or an amino acid sequence at least 90, 95, 95, 96, 97, 98, or 99 percent identical to the amino acid sequence as set forth in SEQ ID NOs: 10 and 12.

In some embodiments, a recombinant antibody or antigen binding fragment thereof comprising an immunoglobulin variable region heavy chain and an immunoglobulin variable region light chain as set forth in SEQ ID NOs: 13 and 15 comprises one or more amino acid sequence mutations back to a germline amino acid sequence, thereby yielding a germline reverted or germlined anti-CD30L antibody. In certain embodiments, the germline reverted or germlined anti-CD30L antibody comprises the amino acid sequence as set forth in SEQ ID NOs: 14 and 16, or an amino acid sequence at least 90, 95, 95, 96, 97, 98, or 99 percent identical to the amino acid sequence as set forth in SEQ ID NOs: 14 and 16.

### 4.3 Cysteine Engineering

In some instances, exposed (for example, solvent exposed) thiol moieties (for example, the thiol moiety of a cysteine amino acid residue) contribute to protein aggregation. For example, disulfide linkages between one or more proteins can be formed as a result of thiol oxidation. In certain instances, mutation of solvent exposed cysteine residues (for example, mutation of one or more solvent exposed cysteine residues) are advantageous in reducing protein aggregation, thereby increasing protein solubility.

Generally, solubility references the ability of a protein to remain dispersed within an aqueous solution (for example, the amount of a monomeric protein species in a solution). In certain instances, the solubility of a protein in an aqueous formulation depends upon the proper distribution of amino acid residues and therefore, solubility can, in certain instances, correlate with the production of correctly folded proteins or the production of proteins retaining. Methods of detecting an increase or decrease in solubility of a protein (for example, an antibody) are known in the art (for example, using routine HPLC techniques, dynamic light scattering techniques, etc.).

Accordingly, in some embodiments, one or more cysteine amino acid residues of the anti-CD30L antibodies are mutated to a non-cysteine amino acid residue (for example, serine). In certain embodiments, the recombinant antibody or antigen binding fragment thereof comprises a heavy chain variable region and/or light chain variable region comprising 1 cysteine residue that is mutated to a non-cysteine residue to 5 cysteine residues that is mutated to a non-cysteine residue. In certain embodiments, the recombinant antibody or antigen binding fragment thereof comprises a heavy chain variable region and/or light chain variable region comprising at least 1 cysteine residue that is mutated to a non-cysteine residue. In certain embodiments, the recombinant antibody or antigen binding fragment thereof comprises a heavy chain variable region and/or light chain variable region comprising at most 5 cysteine residues that is mutated to a non-cysteine residue. In certain embodiments, the recombinant antibody or antigen binding fragment thereof comprises a heavy chain variable region and/or light chain variable region comprising 1 cysteine residue that is mutated to a non-cysteine residue to 2 cysteine residues that is mutated to a non-cysteine residue, 1 cysteine residue that is mutated to a non-cysteine residue to 3 cysteine residues that is mutated to a non-cysteine residue, 1 cysteine residue that is mutated to a non-cysteine residue to 4 cysteine residues that is mutated to a non-cysteine residue, 1 cysteine residue that is mutated to a non-cysteine residue to 5 cysteine residues that is mutated to a non-cysteine residue, 2 cysteine residues that is mutated to a non-cysteine residue to 3 cysteine residues that is mutated to a non-cysteine residue, 2 cysteine residues that is mutated to a non-cysteine residue to 4 cysteine residues that is mutated to a non-cysteine residue, 2 cysteine residues that is mutated to a non-cysteine residue to 5 cysteine residues that is mutated to a non-cysteine residue, 3 cysteine residues that is mutated to a non-cysteine residue to 4 cysteine residues that is mutated to a non-cysteine residue, 3 cysteine residues that is mutated to a non-cysteine residue to 5 cysteine residues that is mutated to a non-cysteine residue, or 4 cysteine residues that is mutated to a non-cysteine residue to 5 cysteine residues that is mutated to a non-cysteine residue. In certain embodiments, the recombinant antibody or antigen binding fragment thereof comprises a heavy chain variable region and/or light chain variable region comprising 1 cysteine residue that is mutated to a non-cysteine residue, 2 cysteine residues that is mutated to a non-cysteine residue, 3 cysteine residues that is mutated to a non-cysteine residue, 4 cysteine residues that is mutated to a non-cysteine residue, or 5 cysteine residues that is mutated to a non-cysteine residue. In some embodiments, one or more cysteine amino acid residues of the anti-CD30L antibody as set forth in SEQ ID NOs: 1-16 are mutated to a non-cysteine amino acid residue.

### 4.4 Fc Constant Regions

Generally, the fragment crystallizable (Fc) region or domain of an antibody (for example, an anti-CD30L antibody) mediates downstream effector functions via its interaction with Fc-receptors on immune cells (for example, innate immune cells) or with complement protein C1q, the recognition molecule of the complement system. Furthermore, the interaction with Fc-receptors can lead to killing of targeted cells through a variety of immune effector mechanisms, including antibody-dependent cell-mediated cytotoxicity (ADCC) and antibody-dependent cellular phagocytosis (ADCP), and antibody-mediated complement activation may lead to complement-dependent cytotoxicity (CDC). In addition, both Fc-receptor interactions and complement activation can exert a broad range of immunomodulatory functions.

Accordingly, in certain instances, mutations within the Fc region that reduce, inhibit, ablate, and/or abrogate Fc-mediated function (for example, ADCC, ADCP, CDC, etc.) of an anti-CD30L antibodies are advantageous for reducing immune activation resulting from the binding of an anti-CD30L antibody to CD30L. In some embodiments, one or more amino acid modifications may be introduced into the Fc region of an anti-CD30L antibody, thereby generating an Fc region variant. An Fc region may comprise a C-terminal region of an immunoglobulin heavy chain that comprises a hinge region, CH2 domain, CH3 domain, or any combination thereof. As used herein, an Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution, addition, or deletion) at one or more amino acid positions.

In some embodiments, the anti-CD30L antibodies of this disclosure are variants that possess some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity) but retains FcRn binding ability. In some embodiments, antibodies are tested for binding to Fcγ receptors and complement C1q by an ELISA based assay, flow cytometry, SPR, or other methods know to interrogate protein-protein interactions and affinity. In some embodiments, antibodies can be tested for reduced effector functions their ability to activate appropriate cell lines or primary human immune cells in vitro, for example, by assessing their ability to induce expression of activation markers, cytokine/chemokine secretion, proliferation, or cell-killing (e.g. using labeled target cells).

In some embodiments, the anti-CD30L antibodies of this disclosure have a reduced effector function as compared to a human IgG. In general, effector function refers to a biological event resulting from the interaction of an antibody Fc region with an Fc receptor or ligand. In certain instances, non-limiting effector functions include C1q binding, complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (for example, B cell receptor), and B cell activation. In some embodiments, antibody-dependent cell-mediated cytotoxicity (ADCC) refers to a cell-mediated reaction in which nonspecific cytotoxic cells expressing Fc receptors (e.g., natural killer cells, neutrophils, macrophages) recognize bound antibody on a target cell, subsequently causing lysis of the target cell. In some embodiments, complement dependent cytotoxicity (CDC) refers to lysing of a target cells in the presence of complement, where the complement action pathway is initiated by the binding of C1q to antibody bound with the target.

Some Fc regions have a natural lack of effector function, and some Fc regions can comprise mutations that reduce effector functions. For instance, IgG4 has low ADCC and CDC activities and IgG2 has low ADCC activity.

In some embodiments, the anti-CD30L antibodies comprise Fc regions characterized by ADCC that is reduced by at least about 30%, at least about 40%, at least about 50%, at least about 60%, at least about 70% or more as compared to an antibody comprising a non-variant Fc region, such as, an antibody with the same sequence identity but for the substitution(s) that decrease ADCC. The disclosure provides antibodies comprising Fc regions characterized by exhibiting CDC that is reduced by at least about 30%, at least about 40%, at least about 50%, at least about 60%, at least about 70% or more as compared to an antibody comprising a non-variant Fc region, i.e., an antibody with the same sequence identity but for the substitution(s) that decrease CDC. In certain embodiments, the antibodies of this disclosure have reduced effector function as compared with human IgG1.

Non-limiting examples of Fc mutations in IgG1 that may reduce ADCC and/or CDC include substitutions at one or more of positions: 231, 232, 234, 235, 236, 237, 238, 239, 264, 265, 267, 269, 270, 297, 299, 318, 320, 322, 325, 327, 328, 329, 330, and 331 in IgG1, where the numbering system of the constant region is that of the EU index as set forth by Kabat. In certain embodiments, the antibodies of this disclosure have reduced effector function as compared with human IgG1.

In certain embodiments, the recombinant anti-CD30L antibody comprises an IgG1 Fc region comprising an N297A substitution, according to the EU numbering system. In certain embodiments, the recombinant anti-CD30L antibody comprises an IgG1 Fc region comprising an N297Q substitution, according to the EU numbering system. In certain embodiments, the recombinant anti-CD30L antibody comprises an IgG1 Fc region comprising an N297D substitution, according to the EU numbering system. In certain embodiments, the recombinant anti-CD30L antibody comprises an IgG1 Fc region comprising an D265A substitution, according to the EU numbering system. In certain embodiments, the recombinant anti-CD30L antibody comprises an IgG1 Fc region comprising an S228P substitution, according to the EU numbering system. In certain embodiments, the recombinant anti-CD30L antibody comprises an IgG1 Fc region comprising an L235A substitution, according to the EU numbering system. In certain embodiments, the recombinant anti-CD30L antibody comprises an IgG1 Fc region comprising an L237A substitution, according to the EU numbering system. In certain embodiments, the recombinant anti-CD30L antibody comprises an IgG1 Fc region comprising an L234A substitution, according to the EU numbering system. In certain embodiments, the recombinant anti-CD30L antibody comprises an IgG1 Fc region comprising an E233P substitution, according to the EU numbering system. In certain embodiments, the recombinant anti-CD30L antibody comprises an IgG1 Fc region comprising an L234V substitution, according to the EU numbering system. In certain embodiments, the recombinant anti-CD30L antibody comprises an IgG1 Fc region comprising an C236 deletion, according to the EU numbering system. In certain embodiments, the recombinant anti-CD30L antibody comprises an IgG1 Fc region comprising a P238A substitution, according to the EU numbering system. In certain embodiments, the recombinant anti-CD30L antibody comprises an IgG1 Fc region comprising an A327Q substitution, according to the EU numbering system. In certain embodiments, the recombinant anti-CD30L antibody comprises an IgG1 Fc region comprising a P329A substitution, according to the EU numbering system. In certain embodiments, the recombinant anti-CD30L antibody comprises an IgG1 Fc region comprising an P329G substitution, according to the EU numbering system. In certain embodiments, the recombinant anti-CD30L antibody comprises an IgG1 Fc region comprising an L235E substitution, according to the EU numbering system. In certain embodiments, the recombinant anti-CD30L antibody comprises an IgG1 Fc region comprising an P331S substitution, according to the EU numbering system. In certain embodiments, the recombinant anti-CD30L antibody comprises an IgG1 Fc region comprising an L234F substitution, according to the EU numbering system. In certain embodiments, the recombinant anti-CD30L antibody comprises an IgG1 Fc region comprising a 235G substitution, according to the EU numbering system. In certain embodiments, the recombinant anti-CD30L antibody comprises an IgG1 Fc region comprising an 235Q substitution, according to the EU numbering system. In certain embodiments, the recombinant anti-CD30L antibody comprises an IgG1 Fc region comprising an 235R substitution, according to the EU numbering system. In certain embodiments, the recombinant anti-CD30L antibody comprises an IgG1 Fc region comprising an 235S substitution, according to the EU numbering system. In certain embodiments, the recombinant anti-CD30L antibody comprises an IgG1 Fc region comprising an 236F substitution, according to the EU numbering system. In certain embodiments, the recombinant anti-CD30L antibody comprises an IgG1 Fc region comprising an 236R substitution, according to the EU numbering system. In certain embodiments, the recombinant anti-CD30L antibody comprises an IgG1 Fc region comprising an 237E substitution, according to the EU numbering system. In certain embodiments, the recombinant anti-CD30L antibody comprises an IgG1 Fc region comprising an 237K substitution, according to the EU numbering system. In certain embodiments, the recombinant anti-CD30L antibody comprises an IgG1 Fc region comprising an 237N substitution, according to the EU numbering system. In certain embodiments, the recombinant anti-CD30L antibody comprises an IgG1 Fc region comprising an 237R substitution, according to the EU numbering system. In certain embodiments, the recombinant anti-CD30L antibody comprises an IgG1 Fc region comprising an 238A substitution, according to the EU numbering system. In certain embodiments, the recombinant anti-CD30L antibody comprises an IgG1 Fc region comprising an 238E substitution, according to the EU numbering system. In certain embodiments, the recombinant anti-CD30L antibody comprises an IgG1 Fc region comprising an 238G substitution, according to the EU numbering system. In certain embodiments, the recombinant anti-CD30L antibody comprises an IgG1 Fc region comprising an 238H substitution, according to the EU numbering system. In certain embodiments, the recombinant anti-CD30L antibody comprises an IgG1 Fc region comprising an 238I substitution, according to the EU numbering system. In certain embodiments, the recombinant anti-CD30L antibody comprises an IgG1 Fc region comprising an 238V substitution, according to the EU numbering system. In certain embodiments, the recombinant anti-CD30L antibody comprises an IgG1 Fc region comprising an 238W substitution, according to the EU numbering system. In certain embodiments, the recombinant anti-CD30L antibody comprises an IgG1 Fc region comprising an 238Y substitution, according to the EU numbering system. In certain embodiments, the recombinant anti-CD30L antibody comprises an IgG1 Fc region comprising an 248A substitution, according to the EU numbering system. In certain embodiments, the recombinant anti-CD30L antibody comprises an IgG1 Fc region comprising an 254D substitution, according to the EU numbering system. In certain embodiments, the recombinant anti-CD30L antibody comprises an IgG1 Fc region comprising an 254E substitution, according to the EU numbering system. In certain embodiments, the recombinant anti-CD30L antibody comprises an IgG1 Fc region comprising an 254G substitution, according to the EU numbering system. In certain embodiments, the recombinant anti-CD30L antibody comprises an IgG1 Fc region comprising an 254H substitution, according to the EU numbering system. In certain embodiments, the recombinant anti-CD30L antibody comprises an IgG1 Fc region comprising an 254I substitution, according to the EU numbering system. In certain embodiments, the recombinant anti-CD30L antibody comprises an IgG1 Fc region comprising an 254N substitution, according to the EU numbering system. In certain embodiments, the recombinant anti-CD30L antibody comprises an IgG1 Fc region comprising an 254P substitution, according to the EU numbering system. In certain embodiments, the recombinant anti-CD30L antibody comprises an IgG1 Fc region comprising an 254Q substitution, according to the EU numbering system. In certain embodiments, the recombinant anti-CD30L antibody comprises an IgG1 Fc region comprising an 254T substitution, according to the EU numbering system. In certain embodiments, the recombinant anti-CD30L antibody comprises an IgG1 Fc region comprising an 254V substitution, according to the EU numbering system. In certain embodiments, the recombinant anti-CD30L antibody comprises an IgG1 Fc region comprising an 255N substitution, according to the EU numbering system. In certain embodiments, the recombinant anti-CD30L antibody comprises an IgG1 Fc region comprising an 256H substitution, according to the EU numbering system. In certain embodiments, the recombinant anti-CD30L antibody comprises an IgG1 Fc region comprising an 256K substitution, according to the EU numbering system. In certain embodiments, the recombinant anti-CD30L antibody comprises an IgG1 Fc region comprising an 256R substitution, according to the EU numbering system. In certain embodiments, the recombinant anti-CD30L antibody comprises an IgG1 Fc region comprising an 256V substitution, according to the EU numbering system. In certain embodiments, the recombinant anti-CD30L antibody comprises an IgG1 Fc region comprising an 264S substitution, according to the EU numbering system. In certain embodiments, the recombinant anti-CD30L antibody comprises an IgG1 Fc region comprising an 265H substitution, according to the EU numbering system. In certain embodiments, the recombinant anti-CD30L antibody comprises an IgG1 Fc region comprising an 265K substitution, according to the EU numbering system. In certain embodiments, the recombinant anti-CD30L antibody comprises an IgG1 Fc region comprising an 265S substitution, according to the EU numbering system. In certain embodiments, the recombinant anti-CD30L antibody comprises an IgG1 Fc region comprising an 265Y substitution, according to the EU numbering system. In certain embodiments, the recombinant anti-CD30L antibody comprises an IgG1 Fc region comprising an 267G substitution, according to the EU numbering system. In certain embodiments, the recombinant anti-CD30L antibody comprises an IgG1 Fc region comprising an 267H substitution, according to the EU numbering system. In certain embodiments, the recombinant anti-CD30L antibody comprises an IgG1 Fc region comprising an 267I substitution, according to the EU numbering system. In certain embodiments, the recombinant anti-CD30L antibody comprises an IgG1 Fc region comprising an 267K substitution, according to the EU numbering system. In certain embodiments, the recombinant anti-CD30L antibody comprises an IgG1 Fc region comprising an 268K substitution, according to the EU numbering system. In certain embodiments, the recombinant anti-CD30L antibody comprises an IgG1 Fc region comprising an 269N substitution, according to the EU numbering system. In certain embodiments, the recombinant anti-CD30L antibody comprises an IgG1 Fc region comprising an 269Q substitution, according to the EU numbering system. In certain embodiments, the recombinant anti-CD30L antibody comprises an IgG1 Fc region comprising an 270A substitution, according to the EU numbering system. In certain embodiments, the recombinant anti-CD30L antibody comprises an IgG1 Fc region comprising an 270G substitution, according to the EU numbering system. In certain embodiments, the recombinant anti-CD30L antibody comprises an IgG1 Fc region comprising an 270M substitution, according to the EU numbering system. In certain embodiments, the recombinant anti-CD30L antibody comprises an IgG1 Fc region comprising an 270N substitution, according to the EU numbering system. In certain embodiments, the recombinant anti-CD30L antibody comprises an IgG1 Fc region comprising an 271T substitution, according to the EU numbering system. In certain embodiments, the recombinant anti-CD30L antibody comprises an IgG1 Fc region comprising an 272N substitution, according to the EU numbering system. In certain embodiments, the recombinant anti-CD30L antibody comprises an IgG1 Fc region comprising an 279F substitution, according to the EU numbering system. In certain embodiments, the recombinant anti-CD30L antibody comprises an IgG1 Fc region comprising an 279K substitution, according to the EU numbering system. In certain embodiments, the recombinant anti-CD30L antibody comprises an IgG1 Fc region comprising an 279L substitution, according to the EU numbering system. In certain embodiments, the recombinant anti-CD30L antibody comprises an IgG1 Fc region comprising an 292E substitution, according to the EU numbering system. In certain embodiments, the recombinant anti-CD30L antibody comprises an IgG1 Fc region comprising an 292F substitution, according to the EU numbering system. In certain embodiments, the recombinant anti-CD30L antibody comprises an IgG1 Fc region comprising an 292G substitution, according to the EU numbering system. In certain embodiments, the recombinant anti-CD30L antibody comprises an IgG1 Fc region comprising an 292I substitution, according to the EU numbering system. In certain embodiments, the recombinant anti-CD30L antibody comprises an IgG1 Fc region comprising an 293S substitution, according to the EU numbering system. In certain embodiments, the recombinant anti-CD30L antibody comprises an IgG1 Fc region comprising an 301W substitution, according to the EU numbering system. In certain embodiments, the recombinant anti-CD30L antibody comprises an IgG1 Fc region comprising an 304E substitution, according to the EU numbering system. In certain embodiments, the recombinant anti-CD30L antibody comprises an IgG1 Fc region comprising an 311E substitution, according to the EU numbering system. In certain embodiments, the recombinant anti-CD30L antibody comprises an IgG1 Fc region comprising an 311G substitution, according to the EU numbering system. In certain embodiments, the recombinant anti-CD30L antibody comprises an IgG1 Fc region comprising an 311S substitution, according to the EU numbering system. In certain embodiments, the recombinant anti-CD30L antibody comprises an IgG1 Fc region comprising an 316F substitution, according to the EU numbering system. In certain embodiments, the recombinant anti-CD30L antibody comprises an IgG1 Fc region comprising an 327T substitution, according to the EU numbering system. In certain embodiments, the recombinant anti-CD30L antibody comprises an IgG1 Fc region comprising an 328V substitution, according to the EU numbering system. In certain embodiments, the recombinant anti-CD30L antibody comprises an IgG1 Fc region comprising an 329Y substitution, according to the EU numbering system. In certain embodiments, the recombinant anti-CD30L antibody comprises an IgG1 Fc region comprising an 330R substitution, according to the EU numbering system. In certain embodiments, the recombinant anti-CD30L antibody comprises an IgG1 Fc region comprising an 339E substitution, according to the EU numbering system. In certain embodiments, the recombinant anti-CD30L antibody comprises an IgG1 Fc region comprising an 339L substitution, according to the EU numbering system. In certain embodiments, the recombinant anti-CD30L antibody comprises an IgG1 Fc region comprising an 343I substitution, according to the EU numbering system. In certain embodiments, the recombinant anti-CD30L antibody comprises an IgG1 Fc region comprising an 343V substitution, according to the EU numbering system. In certain embodiments, the recombinant anti-CD30L antibody comprises an IgG1 Fc region comprising an 373A substitution, according to the EU numbering system. In certain embodiments, the recombinant anti-CD30L antibody comprises an IgG1 Fc region comprising an 373G substitution, according to the EU numbering system. In certain embodiments, the recombinant anti-CD30L antibody comprises an IgG1 Fc region comprising an 373S substitution, according to the EU numbering system. In certain embodiments, the recombinant anti-CD30L antibody comprises an IgG1 Fc region comprising an 376E substitution, according to the EU numbering system. In certain embodiments, the recombinant anti-CD30L antibody comprises an IgG1 Fc region comprising an 376W substitution, according to the EU numbering system. In certain embodiments, the recombinant anti-CD30L antibody comprises an IgG1 Fc region comprising an 376Y substitution, according to the EU numbering system. In certain embodiments, the recombinant anti-CD30L antibody comprises an IgG1 Fc region comprising an 380D substitution, according to the EU numbering system. In certain embodiments, the recombinant anti-CD30L antibody comprises an IgG1 Fc region comprising an 382D substitution, according to the EU numbering system. In certain embodiments, the recombinant anti-CD30L antibody comprises an IgG1 Fc region comprising an 382P substitution, according to the EU numbering system. In certain embodiments, the recombinant anti-CD30L antibody comprises an IgG1 Fc region comprising an 385P substitution, according to the EU numbering system. In certain embodiments, the recombinant anti-CD30L antibody comprises an IgG1 Fc region comprising an 424H substitution, according to the EU numbering system. In certain embodiments, the recombinant anti-CD30L antibody comprises an IgG1 Fc region comprising an 424M substitution, according to the EU numbering system. In certain embodiments, the recombinant anti-CD30L antibody comprises an IgG1 Fc region comprising an 424V substitution, according to the EU numbering system. In certain embodiments, the recombinant anti-CD30L antibody comprises an IgG1 Fc region comprising an 434I substitution, according to the EU numbering system. In certain embodiments, the recombinant anti-CD30L antibody comprises an IgG1 Fc region comprising an 438G substitution, according to the EU numbering system. In certain embodiments, the recombinant anti-CD30L antibody comprises an IgG1 Fc region comprising an 439E substitution, according to the EU numbering system. In certain embodiments, the recombinant anti-CD30L antibody comprises an IgG1 Fc region comprising an 439H substitution, according to the EU numbering system. In certain embodiments, the recombinant anti-CD30L antibody comprises an IgG1 Fc region comprising an 439Q substitution, according to the EU numbering system. In certain embodiments, the recombinant anti-CD30L antibody comprises an IgG1 Fc region comprising an 440A substitution, according to the EU numbering system. In certain embodiments, the recombinant anti-CD30L antibody comprises an IgG1 Fc region comprising an 440D substitution, according to the EU numbering system. In certain embodiments, the recombinant anti-CD30L antibody comprises an IgG1 Fc region comprising an 440E substitution, according to the EU numbering system. In certain embodiments, the recombinant anti-CD30L antibody comprises an IgG1 Fc region comprising an 440F substitution, according to the EU numbering system. In certain embodiments, the recombinant anti-CD30L antibody comprises an IgG1 Fc region comprising an 440M substitution, according to the EU numbering system. In certain embodiments, the recombinant anti-CD30L antibody comprises an IgG1 Fc region comprising an 440T Fc region substitution, according to the EU numbering system. In certain embodiments, the recombinant anti-CD30L antibody comprises an IgG1 Fc region comprising an 440V substitution, according to the EU numbering system.

In certain embodiments, the recombinant anti-CD30L antibody comprises a Fc region selected from the representative sequences disclosed in Table 1.

TABLE 1
Mutations Abrogating Effector Function

| Mutation(s) | Effect |
|---|---|
| E233P | Decreases binding to FcγRI, II, III |
| S228P, L235E SPLE in IgG4 | Decreases binding to FcγRI |
| L235E | Decreases binding to FcγRs |
| L234A, L235A | Decreases binding to FcγRI, II, III |
| L234A, L235A, G237A | Decreases binding to FcγRI, II, III, C1q |
| L234A, L235A, P329G | Decreases binding to FcγRI, II, III, C1q |
| L234F, L235E, P331S | Decreases binding to FcγRI, II, III, C1q |
| L234A, L235E, G237A | Decreases binding to FcγRI, II, III, C1q |
| L234A, L235E, G237A, P331S | Decreases binding to FcγRI, II, III, C1q |
| L234A, L235A, G237A, P238S, | Decreases binding to FcγRI, IIa, II, IIIa |
| H268A, A330S, P331S (IgG1σ) | |
| L234A, L235A, P329A | Decreases binding to FcγRI, II, III, C1q |
| G236R, L328R | Decreases binding to FcγRI, II, III |
| G237A | Decreases binding to FcγRII |
| F241A | Decreases binding to C1q |
| V264A | Decreases binding to C1q |
| D265A | Decreases binding to FcγRI, II, III |
| D265A, N297A | Decreases binding to FcγRI, II, III, C1q |
| D265A, N297G | Decreases binding to FcγRI, II, III, C1q |
| D270A | Decreases binding to C1q |
| N297A, G, D, Q | Elimination of N-linked glycosylation |
| | Decreases binding to FcγRI, II, III, C1q |
| P329A, G, R | Decreases binding to C1q |
| A330L | Decreases binding to C1q |
| P331A, S | Diminished C1q binding |
| IgG2 | Decreases binding to FcγRs |
| IgG4 | Decreases binding to FcγRs; |
| | Does not activate complement system |
| S228P | Prevent IgG4 Fab arm exchange |
| S228P, F234A, L235A (IgG4) | Decreases binding to FcγRI, IIa, IIIa |
| IgG2-IgG4 cross-subclass | Decreases binding to FcγRI, II, III, C1q |
| (IgG2/G4) | |
| IgG2-IgG3 cross-subclass | Decreases binding to FcγRs; |
| | Decreases binding to C1q |
| H268Q, V309L, A330S, P331S | Decreases binding to FcγRI, II, III, C1q |
| (IgG2m4) | |
| V234A, G237A, P238S, H268A, | Decreases binding to FcγRI, IIa, IIb, |
| V309L, A330S, P331S (IgG2σ) | IIIa, C1q |
| High mannose glycosylation | Decreases binding to C1q |

In certain embodiments, the recombinant anti-CD30L antibody comprises an IgG1 Fc region comprising E233P, according to the EU numbering system. In certain embodiments, the recombinant anti-CD30L antibody comprises an IgG4 Fc region comprising S228P and L235E. In certain embodiments, the recombinant anti-CD30L antibody comprises an IgG1 Fc region comprising L235E, according to the EU numbering system. In certain embodiments, the recombinant anti-CD30L antibody comprises an IgG1 Fc region comprising L234A and L235A, according to the EU numbering system. In certain embodiments, the recombinant anti-CD30L antibody comprises an IgG1 Fc region comprising L234A, L235A, and G237A, according to the EU numbering system. In certain embodiments, the recombinant anti-CD30L antibody comprises an IgG1 Fc region comprising L234A, L235A, P329G, according to the EU numbering system. In certain embodiments, the recombinant anti-CD30L antibody comprises an IgG1 Fc region comprising L234F, L235E, and P331S, according to the EU numbering system. In certain embodiments, the recombinant anti-CD30L antibody comprises an IgG1 Fc region comprising L234A, L235E, and G237A, according to the EU numbering system. In certain embodiments, the recombinant anti-CD30L antibody comprises an IgG1 Fc region comprising L234A, L235E, G237A, and P331S, according to the EU numbering system. In certain embodiments, the recombinant anti-CD30L antibody comprises an IgG1 Fc region comprising L234A, L235A, G237A, P238S, H268A, A330S, and P331S (IgG1), according to the EU numbering system. In certain embodiments, the recombinant anti-CD30L antibody comprises an IgG1 Fc region comprising L234A, L235A, and P329A, according to the EU numbering system. In certain embodiments, the recombinant anti-CD30L antibody comprises an IgG1 Fc region comprising G236R and L328R, according to the EU numbering system. In certain embodiments, the recombinant anti-CD30L antibody comprises an IgG1 Fc region comprising G237A, according to the EU numbering system. In certain embodiments, the recombinant anti-CD30L antibody comprises an IgG1 Fc region comprising F241A, according to the EU numbering system. In certain embodiments, the recombinant anti-CD30L antibody comprises an IgG1 Fc region comprising V264A, according to the EU numbering system. In certain embodiments, the recombinant anti-CD30L antibody comprises an IgG1 Fc region comprising D265A, according to the EU numbering system. In certain embodiments, the recombinant anti-CD30L antibody comprises an IgG1 Fc region comprising D265A and N297A, according to the EU numbering system. In certain embodiments, the recombinant anti-CD30L antibody comprises an IgG1 Fc region comprising D265A and N297G, according to the EU numbering system. In certain embodiments, the recombinant anti-CD30L antibody comprises an IgG1 Fc region comprising D270A, according to the EU numbering system. In certain embodiments, the recombinant anti-CD30L antibody comprises an IgG1 Fc region comprising N297A, according to the EU numbering system. In certain embodiments, the recombinant anti-CD30L antibody comprises an IgG1 Fc region comprising N297G, according to the EU numbering system. In certain embodiments, the recombinant anti-CD30L antibody comprises an IgG1 Fc region comprising N297D, according to the EU numbering system. In certain embodiments, the recombinant anti-CD30L antibody comprises an IgG1 Fc region comprising N297Q, according to the EU numbering system. In certain embodiments, the recombinant anti-CD30L antibody comprises an IgG1 Fc region comprising P329A, according to the EU numbering system. In certain embodiments, the recombinant anti-CD30L antibody comprises an IgG1 Fc region comprising P329G, according to the EU numbering system. In certain embodiments, the recombinant anti-CD30L antibody comprises an IgG1 Fc region comprising P329R, according to the EU numbering system. In certain embodiments, the recombinant anti-CD30L antibody comprises an IgG1 Fc region comprising A330L, according to the EU numbering system. In certain embodiments, the recombinant anti-CD30L antibody comprises an IgG1 Fc region comprising P331A, according to the EU numbering system. In certain embodiments, the recombinant anti-CD30L antibody comprises an IgG1 Fc region comprising P331S, according to the EU numbering system. In certain embodiments, the recombinant anti-CD30L antibody comprises an IgG2 Fc region. In certain embodiments, the recombinant anti-CD30L antibody comprises an IgG4 Fc region. In certain embodiments, the recombinant anti-CD30L antibody comprises an IgG4 Fc region comprising S228P, according to the EU numbering system. In certain embodiments, the recombinant anti-CD30L antibody comprises an IgG4 Fc region comprising S228P, F234A, and L235A, according to the EU numbering system. In certain embodiments, the recombinant anti-CD30L antibody comprises an IgG2-IgG4 cross-subclass (IgG2/G4) Fc region. In certain embodiments, the recombinant anti-CD30L antibody comprises an IgG2-IgG3 cross-subclass Fc region. In certain embodiments, the recombinant anti-CD30L antibody comprises an IgG2 Fc region comprising H268Q, V309L, A330S, and P331S, according to the EU numbering system. In certain embodiments, the recombinant anti-CD30L antibody comprises an IgG2 Fc region comprising V234A, G237A, P238S, H268A, V309L, A330S, and P331S, according to the EU numbering system. In certain embodiments, the recombinant anti-CD30L antibody comprises a Fc region comprising high mannose glycosylation.

In certain embodiments, the recombinant anti-CD30L antibody comprises an IgG4 Fc region comprising a S228P substitution, according to the EU numbering system. In certain embodiments, the recombinant anti-CD30L antibody comprises an IgG4 Fc region comprising an A330S substitution, according to the EU numbering system. In certain embodiments, the recombinant anti-CD30L antibody comprises an IgG4 Fc region comprising a P331S substitution, according to the EU numbering system.

In certain embodiments, the recombinant anti-CD30L antibody comprises an IgG2 Fc region comprising an A330S substitution, according to the EU numbering system. In certain embodiments, the recombinant anti-CD30L antibody comprises an IgG2 Fc region comprising an P331S substitution, according to the EU numbering system. In certain embodiments, the recombinant anti-CD30L antibody comprises an IgG2 Fc region comprising an 234A substitution, according to the EU numbering system. In certain embodiments, the recombinant anti-CD30L antibody comprises an IgG2 Fc region comprising an 237A substitution, according to the EU numbering system.

In some embodiments, assessment of ADCC activity of an anti-CD30L antibody comprises adding the antibody to target cells in combination with immune effector cells, which may be activated by the antigen-antibody complexes resulting in cytolysis of the target cell. Cytolysis may be detected by the release of label (for example, radioactive substrates, fluorescent dyes or natural intracellular proteins) from the lysed cells. Useful effector cells for such assays include peripheral blood mononuclear cells (PBMC) and Natural Killer (NK) cells. Specific examples of in vitro ADCC assays are described in Wisecarver et al., 1985 79:277-282; Bruggemann et al., 1987, J Exp Med 166:1351-1361; Wilkinson et al., 2001, J Immunol Methods 258:183-191; Patel et al., 1995 J Immunol Methods 184:29-38. Alternatively, or additionally, ADCC activity of the antibody of interest may be assessed in vivo, e.g., in an animal model such as that disclosed in Clynes et al., 1998, PNAS USA 95:652-656.

In some embodiments, antibodies comprising a Fc region herein exhibit decreased ADCC activities as compared to an unmodified antibody (e.g., an antibody with human IgG1). In some embodiments, the antibodies herein exhibit ADCC activities that are at least 2-fold, or at least 3-fold, or at least 5-fold or at least 10-fold or at least 50-fold or at least 100-fold less than that of an unmodified antibody. In some embodiments, antibodies herein exhibit ADCC activities that are reduced by at least 10%, or at least 20%, or by at least 30%, or by at least 40%, or by at least 50%, or by at least 60%, or by at least 70%, or by at least 80%, or by at least 90%, or by at least 100%, or by at least 200%, or by at least 300%, or by at least 400%, or by at least 500% relative to an unmodified antibody. In certain embodiments, antibodies herein have no detectable ADCC activity. In certain embodiments, the reduction and/or ablatement of ADCC activity may be attributed to the reduced affinity antibodies of the invention exhibit for Fc ligands and/or receptors.

In some embodiments, an assessment of complement activation, a CDC assay, may be performed as described in Gazzano-Santoro et al., 1996, J. Immunol. Methods, 202:163. In some embodiments, antibodies comprising Fc regions described herein exhibit decreased affinities to C1q relative to an unmodified antibody (e.g., human IgG1). In some embodiments, antibodies herein exhibit affinities for C1q receptor that are at least 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold less than an unmodified antibody. In some embodiments, antibodies herein exhibit affinities for C1q that are at least 90%, at least 80%, at least 70%, at least 60%, at least 50%, at least 40%, at least 30%, at least 20%, at least 10%, or at least 5% less than an unmodified antibody.

In some embodiments, antibodies comprising Fc regions described herein exhibit decreased CDC activities as compared to an unmodified antibody (e.g., human IgG1). In some embodiments, antibodies herein exhibit CDC activities that are at least 2-fold, or at least 3-fold, or at least 5-fold or at least 10-fold or at least 50-fold or at least 100-fold less than that of an unmodified antibody. In some embodiments, antibodies herein exhibit CDC activities that are reduced by at least 10%, or at least 20%, or by at least 30%, or by at least 40%, or by at least 50%, or by at least 60%, or by at least 70%, or by at least 80%, or by at least 90%, or by at least 100%, or by at least 200%, or by at least 300%, or by at least 400%, or by at least 500% relative to an unmodified antibody. In certain embodiments, antibodies herein exhibit no detectable CDC activities. In some embodiments, the reduction and/or ablatement of CDC activity may be attributed to the reduced affinity antibodies of the invention exhibit for Fc ligands and/or receptors.

Accordingly, further provided and described herein are anti-CD30L antibodies comprising a variant Fc region (for example, harboring mutations) that reduce the cytotoxic response (for example, ADCC or CDC) elicited by an anti-CD30L antibody. In some embodiments, an anti-CD30L antibody described herein comprises a Fc region comprising SEQ ID NO: 500 or a sequence at least about 90%, 91%, 92%, 93%, 94%, 95%, 96%, 97%, 98%, or 99% identical to SEQ ID NO: 500. In some embodiments, an anti-CD30L antibody described herein comprises a Fc region comprising SEQ ID NO: 501 or a sequence at least about 90%, 91%, 92%, 93%, 94%, 95%, 96%, 97%, 98%, or 99% identical to SEQ ID NO: 501. In some embodiments, an anti-CD30L antibody described herein comprises a Fc region comprising SEQ ID NO: 502 or a sequence at least about 90%, 91%, 92%, 93%, 94%, 95%, 96%, 97%, 98%, or 99% identical to SEQ ID NO: 502. In some embodiments, an anti-CD30L antibody described herein comprises a Fc region comprising SEQ ID NO: 503 or a sequence at least about 90%, 91%, 92%, 93%, 94%, 95%, 96%, 97%, 98%, or 99% identical to SEQ ID NO: 503. In some embodiments, an anti-CD30L antibody described herein comprises a Fc region comprising SEQ ID NO: 504 or a sequence at least about 90%, 91%, 92%, 93%, 94%, 95%, 96%, 97%, 98%, or 99% identical to SEQ ID NO: 504. In some embodiments, an anti-CD30L antibody described herein comprises a Fc region comprising SEQ ID NO: 505 or a sequence at least about 90%, 91%, 92%, 93%, 94%, 95%, 96%, 97%, 98%, or 99% identical to SEQ ID NO: 505. In some embodiments, an anti-CD30L antibody described herein comprises a Fc region comprising SEQ ID NO: 506 or a sequence at least about 90%, 91%, 92%, 93%, 94%, 95%, 96%, 97%, 98%, or 99% identical to SEQ ID NO: 506. In some embodiments, an anti-CD30L antibody described herein comprises a Fc region comprising SEQ ID NO: 507 or a sequence at least about 90%, 91%, 92%, 93%, 94%, 95%, 96%, 97%, 98%, or 99% identical to SEQ ID NO: 507. In some embodiments, an anti-CD30L antibody described herein comprises a Fc region comprising SEQ ID NO: 508 or a sequence at least about 90%, 91%, 92%, 93%, 94%, 95%, 96%, 97%, 98%, or 99% identical to SEQ ID NO: 508. In some embodiments, an anti-CD30L antibody described herein comprises a Fc region comprising SEQ ID NO: 509 or a sequence at least about 90%, 91%, 92%, 93%, 94%, 95%, 96%, 97%, 98%, or 99% identical to SEQ ID NO: 509. In some embodiments, an anti-CD30L antibody described herein comprises a Fc region comprising SEQ ID NO: 510 or a sequence at least about 90%, 91%, 92%, 93%, 94%, 95%, 96%, 97%, 98%, or 99% identical to SEQ ID NO: 510. In some embodiments, an anti-CD30L antibody described herein comprises a Fc region comprising SEQ ID NO: 511 or a sequence at least about 90%, 91%, 92%, 93%, 94%, 95%, 96%, 97%, 98%, or 99% identical to SEQ ID NO: 511. In some embodiments, an anti-CD30L antibody described herein comprises a Fc region comprising SEQ ID NO: 512 or a sequence at least about 90%, 91%, 92%, 93%, 94%, 95%, 96%, 97%, 98%, or 99% identical to SEQ ID NO: 512.

In certain embodiments, the recombinant antibody or antigen binding fragment thereof comprises a variant Fc region (for example, harboring mutations) that reduce the cytotoxic response (for example, ADCC or CDC) elicited by an anti-CD30L antibody and wherein the anti-CD30L antibody comprises (a) a CDR-H1 comprising the amino acid sequence set forth in any one of SEQ ID NOs: 100-109; (b) a CDR-H2 comprising the amino acid sequence set forth in any one of SEQ ID NOs: 140-149; (c) an immunoglobulin heavy chain CDR3 (CDR-H3) comprising the amino acid sequence set forth in any one of SEQ ID NOs: 180-189; (d) a CDR-L1 comprising the amino acid sequence set forth in any one of SEQ ID NOs: 300-309; (e) a CDR-L2 comprising the amino acid sequence set forth in any one of SEQ ID NOs: 340-349; and/or (f) a CDR-L3 comprising the amino acid sequence set forth in any one of SEQ ID NOs: 380-389. In certain embodiments, the recombinant antibody or antigen binding fragment thereof comprises a variant Fc region (for example, harboring mutations) that reduce the cytotoxic response (for example, ADCC or CDC) elicited by an anti-CD30L antibody and wherein the anti-CD30L antibody comprises an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to any one of SEQ ID NOs: 1 and 2; and/or (b) the immunoglobulin variable region light chain comprises an amino acid sequence having at least about 90, 95, 97, 98, 99, or 100% sequence identity to any one of SEQ ID NOs: 3 and 4. In certain embodiments, the anti-CD30L antibody comprises a Fc region comprising the amino acid sequence as set forth in any one of SEQ ID NOs: 500-512 or a sequence at least about 90, 91, 92, 93, 94, 95, 96, 97, 98, or 99% identical to SEQ ID NOs: 500-512. In certain embodiments, the anti-CD30L antibody comprises a Fc region comprising one or more of the mutations as set forth in Table 1.

In certain embodiments, the recombinant antibody or antigen binding fragment thereof comprises a variant Fc region (for example, harboring mutations) that reduce the cytotoxic response (for example, ADCC or CDC) elicited by an anti-CD30L antibody and wherein the anti-CD30L antibody comprises (a) a CDR-H1 comprising the amino acid sequence set forth in any one of SEQ ID NOs: 110-119; (b) a CDR-H2 comprising the amino acid sequence set forth in any one of SEQ ID NOs: 150-159; (c) an immunoglobulin heavy chain CDR3 (CDR-H3) comprising the amino acid sequence set forth in any one of SEQ ID NOs: 190-199; (d) a CDR-L1 comprising the amino acid sequence set forth in any one of SEQ ID NOs: 310-319; (e) a CDR-L2 comprising the amino acid sequence set forth in any one of SEQ ID NOs: 350-359; and/or (f) a CDR-L3 comprising the amino acid sequence set forth in any one of SEQ ID NOs: 390-399. In certain embodiments, the recombinant antibody or antigen binding fragment thereof comprises a variant Fc region (for example, harboring mutations) that reduce the cytotoxic response (for example, ADCC or CDC) elicited by an anti-CD30L antibody and wherein the anti-CD30L antibody comprises an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to any one of SEQ ID NOs: 5 and 6; and/or (b) the immunoglobulin variable region light chain comprises an amino acid sequence having at least about 90, 95, 97, 98, 99, or 100% sequence identity to any one of SEQ ID NOs: 7 and 8. In certain embodiments, the anti-CD30L antibody comprises a Fc region comprising the amino acid sequence as set forth in any one of SEQ ID NOs: 500-512 or a sequence at least about 90, 91, 92, 93, 94, 95, 96, 97, 98, or 99% identical to SEQ ID NOs: 500-512. In certain embodiments, the anti-CD30L antibody comprises a Fc region comprising one or more of the mutations as set forth in Table 1.

In certain embodiments, the recombinant antibody or antigen binding fragment thereof comprises a variant Fc region (for example, harboring mutations) that reduce the cytotoxic response (for example, ADCC or CDC) elicited by an anti-CD30L antibody and wherein the anti-CD30L antibody comprises (a) a CDR-H1 comprising the amino acid sequence set forth in any one of SEQ ID NOs: 120-129; (b) a CDR-H2 comprising the amino acid sequence set forth in any one of SEQ ID NOs: 160-169; (c) an immunoglobulin heavy chain CDR3 (CDR-H3) comprising the amino acid sequence set forth in any one of SEQ ID NOs: 200-209; (d) a CDR-L1 comprising the amino acid sequence set forth in any one of SEQ ID NOs: 320-329; (e) a CDR-L2 comprising the amino acid sequence set forth in any one of SEQ ID NOs: 360-369; and/or (f) a CDR-L3 comprising the amino acid sequence set forth in any one of SEQ ID NOs: 400-409. In certain embodiments, the recombinant antibody or antigen binding fragment thereof comprises a variant Fc region (for example, harboring mutations) that reduce the cytotoxic response (for example, ADCC or CDC) elicited by an anti-CD30L antibody and wherein the anti-CD30L antibody comprises an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to any one of SEQ ID NOs: 9 and 10; and/or (b) the immunoglobulin variable region light chain comprises an amino acid sequence having at least about 90, 95, 97, 98, 99, or 100% sequence identity to any one of SEQ ID NOs: 11 and 12. In certain embodiments, the anti-CD30L antibody comprises a Fc region comprising the amino acid sequence as set forth in any one of SEQ ID NOs: 500-512 or a sequence at least about 90, 91, 92, 93, 94, 95, 96, 97, 98, or 99% identical to SEQ ID NOs: 500-512. In certain embodiments, the anti-CD30L antibody comprises a Fc region comprising one or more of the mutations as set forth in Table 1.

In certain embodiments, the recombinant antibody or antigen binding fragment thereof comprises a variant Fc region (for example, harboring mutations) that reduce the cytotoxic response (for example, ADCC or CDC) elicited by an anti-CD30L antibody and wherein the anti-CD30L antibody comprises (a) a CDR-H1 comprising the amino acid sequence set forth in any one of SEQ ID NOs: 130-139; (b) a CDR-H2 comprising the amino acid sequence set forth in any one of SEQ ID NOs: 170-179; (c) an immunoglobulin heavy chain CDR3 (CDR-H3) comprising the amino acid sequence set forth in any one of SEQ ID NOs: 210-219; (d) a CDR-L1 comprising the amino acid sequence set forth in any one of SEQ ID NOs: 330-339; (e) a CDR-L2 comprising the amino acid sequence set forth in any one of SEQ ID NOs: 370-379; and/or (f) a CDR-L3 comprising the amino acid sequence set forth in any one of SEQ ID NOs: 410-419. In certain embodiments, the recombinant antibody or antigen binding fragment thereof comprises a variant Fc region (for example, harboring mutations) that reduce the cytotoxic response (for example, ADCC or CDC) elicited by an anti-CD30L antibody and wherein the anti-CD30L antibody comprises an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to any one of SEQ ID NOs: 13 and 14; and/or (b) the immunoglobulin variable region light chain comprises an amino acid sequence having at least about 90, 95, 97, 98, 99, or 100% sequence identity to any one of SEQ ID NOs: 15 and 16. In certain embodiments, the anti-CD30L antibody comprises a Fc region comprising the amino acid sequence as set forth in any one of SEQ ID NOs: 500-512 or a sequence at least about 90, 91, 92, 93, 94, 95, 96, 97, 98, or 99% identical to SEQ ID NOs: 500-512. In certain embodiments, the anti-CD30L antibody comprises a Fc region comprising one or more of the mutations as set forth in Table 1.

### 4.5 Pharmaceutical Compositions

In one aspect, anti-CD30L antibodies or antigen binding fragments provided herein are formulated into pharmaceutical compositions that are useful in a variety of applications including, but not limited to, therapeutic methods, such as the treatment of IBD. The methods of use may be in vitro, ex vivo, or in vivo methods. In certain embodiments, the disease treated with anti-CD30L antibody or antigen binding fragment is IBD, CD, UC and/or MR-UC.

In one aspect, provided herein are various pharmaceutical compositions comprising an antibody or antigen binding fragment that binds to a CD30L, a CD30L polypeptide fragment, a CD30L peptide, or a CD30L epitope.

As is clear from the descriptions above and below, the anti-CD30L antibody or antigen binding fragment provided herein including in Section 4.1 can be formulated in various pharmaceutical compositions described herein including in this Section 4.5. Accordingly, in various embodiments of the pharmaceutical composition provided herein, including in this Section (Section 4.5), the anti-CD30L antibody or antigen binding fragment in the pharmaceutical composition is provided in Sections 2 and 4.1, the concentrations of the anti-CD30L antibody or antigen binding fragment in the pharmaceutical composition is provided in Section XX, the buffer in the pharmaceutical composition is provided in Section XX, the buffer of the pharmaceutical composition is provided in Section XX, the pharmaceutically acceptable excipient and the concentration of such excipient in the pharmaceutical composition is provided in Section XX, the surfactant and the concentration of such surfactant in the pharmaceutical composition is provided in Section XX, the viscosity of the pharmaceutical composition is provided in Section XX, the stability and the measurement for such stability of the pharmaceutical composition is provided in Section XX. Therefore, the disclosure herein provides various combinations of the anti-CD30L antibody or antigen binding fragment, the therapeutically effective amount (such as the dose and dosing regimens) for the anti-CD30L antibody or antigen binding fragment, the buffer, the excipient, the surfactant, the viscosity, the stability, each further described herein in details, for the pharmaceutical composition.

In various embodiments, the pharmaceutical compositions are formulated for delivery via any route of administration. “Route of administration” includes any administration pathway known in the art, including but not limited to intravenous, subcutaneous, aerosol, nasal, oral, transmucosal, transdermal and parenteral. In example embodiments, the route of administration is subcutaneous.

In certain embodiments, provided herein is a pharmaceutical composition comprising an anti-CD30L antibody or antigen binding fragment thereof including those provided in Sections 2 and 4.1, wherein the pharmaceutical composition is formulated for intravenous administration.

In certain embodiments, provided herein is a pharmaceutical composition comprising an anti-CD30L antibody or antigen binding fragment thereof including those provided in Sections 2 and 4.1, wherein the pharmaceutical composition is formulated for subcutaneous administration.

In one aspect, the present disclosure further provides pharmaceutical compositions comprising at least one anti-CD30L antibody or antigen binding fragment provided herein. In some embodiments, a pharmaceutical composition comprises 1) an anti-CD30L antibody or antigen binding fragment, and 2) a pharmaceutically acceptable carrier.

Pharmaceutical compositions comprising an antibody are prepared for storage by mixing the antibody having the desired degree of purity with optional physiologically acceptable carriers, excipients, or stabilizers (see, e.g., Remington, Remington's Pharmaceutical Sciences (18th ed. 1980)) in the form of aqueous solutions or lyophilized or other dried forms.

The antibodies or antigen binding fragments of the present disclosure can be formulated in any suitable form for delivery to a target cell/tissue, e.g., as microcapsules or macroemulsions (Remington, supra; Park et al., 2005, Molecules 10:146-61; Malik et al., 2007, Curr. Drug. Deliv. 4:141-51), as sustained release formulations (Putney and Burke, 1998, Nature Biotechnol. 16:153-57), or in liposomes (Maclean et al., 1997, Int. J. Oncol. 11:325-32; Kontermann, 2006, Curr. Opin. Mol. Ther. 8:39-45).

An antibody provided herein can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles, and nanocapsules) or in macroemulsions. Such techniques are disclosed, for example, in Remington, supra.

Various compositions and delivery systems are known and can be used with an antibody that binds to CD30L as described herein, including, but not limited to, encapsulation in liposomes, microparticles, microcapsules, recombinant cells capable of expressing the antibody, receptor-mediated endocytosis (see, e.g., Wu and Wu, 1987, J. Biol. Chem. 262:4429-32), construction of a nucleic acid as part of a retroviral or other vector, etc. In another embodiment, a composition can be provided as a controlled release or sustained release system. In one embodiment, a pump can be used to achieve controlled or sustained release (see, e.g., Langer, supra; Sefton, 1987, Crit. Ref. Biomed. Eng. 14:201-40; Buchwald et al., 1980, Surgery 88:507-16; and Saudek et al., 1989, N. Engl. J. Med. 321:569-74). In another embodiment, polymeric materials can be used to achieve controlled or sustained release of a prophylactic or therapeutic agent (e.g., an antibody that binds to CD30L as described herein) or a composition of the invention (see, e.g., Medical Applications of Controlled Release (Langer and Wise eds., 1974); Controlled Drug Bioavailability, Drug Product Design and Performance (Smolen and Ball eds., 1984); Ranger and Peppas, 1983, J. Macromol. Sci. Rev. Macromol. Chem. 23:61-126; Levy et al., 1985, Science 228:190-92; During et al., 1989, Ann. Neurol. 25:351-56; Howard et al., 1989, J. Neurosurg. 71:105-12; U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable.

In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, 1990, Science 249:1527-33. Any technique known to one of skill in the art can be used to produce sustained release formulations comprising one or more antibodies that bind to CD30L as described herein (see, e.g., U.S. Pat. No. 4,526,938, PCT publication Nos. WO 91/05548 and WO 96/20698, Ning et al., 1996, Radiotherapy & Oncology 39:179-89; Song et al., 1995, PDA J. of Pharma. Sci. & Tech. 50:372-97; Cleek et al., 1997, Pro. Int'l. Symp. Control. Rel. Bioact. Mater. 24:853-54; and Lam et al., 1997, Proc. Int'l. Symp. Control Rel. Bioact. Mater. 24:759-60).

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. The term “carrier” can also refer to a diluent, adjuvant (e.g., Freund's adjuvant (complete or incomplete)), excipient, or vehicle. Such carriers, including pharmaceutical carriers, can be sterile liquids, such as water and oils, including those of petroleum, animal, vegetable, or synthetic origin, such as peanut oil, soybean oil, mineral oil, sesame oil, and the like. Water is an exemplary carrier when a composition (e.g., a pharmaceutical composition) is administered intravenously. Saline solutions and aqueous dextrose and glycerol solutions can also be employed as liquid carriers, particularly for injectable solutions. Suitable excipients (e.g., pharmaceutical excipients) include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard carriers such as pharmaceutical grades of mannitol, lactose, starch, magnesium stearate, sodium saccharine, cellulose, magnesium carbonate, etc. Examples of suitable pharmaceutical carriers are described in Remington and Gennaro, Remington's Pharmaceutical Sciences (18th ed. 1990). Compositions, including pharmaceutical compounds, can contain an anti-CD30L antibody, for example, in isolated or purified form, together with a suitable amount of carriers.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

The term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.), and polyols (e.g., mannitol, sorbitol, etc.). See, also, Remington and Gennaro, Remington's Pharmaceutical Sciences (18th ed. 1990), which is hereby incorporated by reference in its entirety. Non-limiting examples include, for example, starch, glucose, lactose, sucrose, gelatin, malt, rice, flour, chalk, silica gel, sodium stearate, glycerol monostearate, talc, sodium chloride, dried skim milk, water, saline, dextrose, propylene glycol, glycerol, ethanol, mannitol, polysorbate or the like and combinations thereof. In addition, if desired, the composition can contain auxiliary substances such as wetting or emulsifying agents, pH buffering agents and the like which enhance or maintain the effectiveness of the active ingredient. The pharmaceutical compositions as described herein can include pharmaceutically acceptable salts. Pharmaceutically acceptable salts include the acid addition salts formed with inorganic acids such as, for example, hydrochloric or phosphoric acids, organic acids, for example, acetic, tartaric or mandelic, salts formed from inorganic bases such as, for example, sodium, potassium, ammonium, calcium or ferric hydroxides, and salts formed from organic bases such as isopropylamine, trimethylamine, 2-ethylamino ethanol, histidine, procaine and the like. Liquid compositions can contain liquid phases in addition to and in the exclusion of water, for example, glycerin, vegetable oils such as cottonseed oil, and water-oil emulsions. Physiologically tolerable carriers are well known in the art. The amount of antibody used that will be effective in the treatment of a particular disorder or condition will depend on the nature of the disorder or condition and can be determined by one of skill in the art with standard clinical techniques.

In one aspect, provided herein is a pharmaceutical composition comprising an antibody or antigen binding fragment thereof that binds CD30L, wherein the antibody or antigen binding fragment thereof comprises: (a) an immunoglobulin heavy chain CDR1 (CDR-H1) comprising the amino acid sequence set forth in any one of SEQ ID NOs: 100-139, 220-234, 465-489, 628-641, and 712-723; (b) an immunoglobulin heavy chain CDR2 (CDR-H2) comprising the amino acid sequence set forth in any one of SEQ ID NOs: 140-179, 235-249, 490-499, 513-527, 642-655, and 724-735; (c) an immunoglobulin heavy chain CDR3 (CDR-H3) comprising the amino acid sequence set forth in any one of SEQ Id NOs: 180-219, 250-264, 528-552, 656-669, and 736-743; (d) an immunoglobulin light chain CDR1 (CDR-L1) comprising the amino acid sequence set forth in any one of SEQ ID NOs: 300-339, 420-434, 553-577, 670-683, and 744-751; (d) an immunoglobulin light chain CDR2 (CDR-L2) comprising the amino acid sequence set forth in any one of SEQ ID NOs: 340-379, 435-449, 578-602, 684-697, and 752-759; and/or (e) an immunoglobulin light chain CDR3 (CDR-L3) comprising the amino acid sequence set forth in any one of SEQ ID NOs: 380-419, 450-464, 603-627, 698-711, and 760-765.

Consistent with the above aspect, in one embodiment, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723. In some embodiments, the pharmaceutical composition comprises an anti-CD30L antibody or antigen binding fragment, wherein the antibody or antigen binding fragment thereof comprises a CDR-H2 comprising the amino acid sequence set forth in any one of SEQ ID NOs: 140-179, 235-249, 490-499, 513-527, 642-655, and 724-735. In certain embodiments, the pharmaceutical composition comprises an anti-CD30L antibody or antigen binding fragment, wherein the antibody or antigen binding fragment thereof comprises a CDR-H3 comprising the amino acid sequence set forth in any one of SEQ Id NOs: 180-219, 250-264, 528-552, 656-669, and 736-743. In other embodiments, the pharmaceutical composition comprises an anti-CD30L antibody or antigen binding fragment, wherein the antibody or antigen binding fragment thereof comprises a CDR-L1 comprising the amino acid sequence set forth in any one of SEQ ID NOs: 300-339, 420-434, 553-577, 670-683, and 744-751. In yet other embodiments, the pharmaceutical composition comprises an anti-CD30L antibody or antigen binding fragment, wherein the antibody or antigen binding fragment thereof comprises a CDR-L2 comprising the amino acid sequence set forth in any one of SEQ ID NOs: 340-379, 435-449, 578-602, 684-697, and 752-759. In another embodiment, the pharmaceutical composition comprises an anti-CD30L antibody or antigen binding fragment, wherein the antibody or antigen binding fragment thereof comprises a CDR-L3 comprising the amino acid sequence set forth in any one of SEQ ID NOs: 380-419, 450-464, 603-627, 698-711, and 760-765. In one embodiment, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723 and a CDR-H2 comprising the amino acid sequence set forth in any one of SEQ ID NOs: 140-179, 235-249, 490-499, 513-527, 642-655, and 724-735. In some embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723 and a CDR-H3 comprising the amino acid sequence set forth in any one of SEQ Id NOs: 180-219, 250-264, 528-552, 656-669, and 736-743. In certain embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723 and a CDR-L1 comprising the amino acid sequence set forth in any one of SEQ ID NOs: 300-339, 420-434, 553-577, 670-683, and 744-751. In other embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723 and a CDR-L2 comprising the amino acid sequence set forth in any one of SEQ ID NOs: 340-379, 435-449, 578-602, 684-697, and 752-759. In some embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723 and a CDR-L3 comprising the amino acid sequence set forth in any one of SEQ ID NOs: 380-419, 450-464, 603-627, 698-711, and 760-765. In further embodiments, the pharmaceutical composition comprises an anti-CD30L antibody or antigen binding fragment, wherein the antibody or antigen binding fragment thereof comprises a CDR-H2 comprising the amino acid sequence set forth in any one of SEQ ID NOs: 140-179, 235-249, 490-499, 513-527, 642-655, and 724-735 and a CDR-H3 comprising the amino acid sequence set forth in any one of SEQ Id NOs: 180-219, 250-264, 528-552, 656-669, and 736-743. In one embodiment, the pharmaceutical composition comprises an anti-CD30L antibody or antigen binding fragment, wherein the antibody or antigen binding fragment thereof comprises a CDR-H2 comprising the amino acid sequence set forth in any one of SEQ ID NOs: 140-179, 235-249, 490-499, 513-527, 642-655, and 724-735 and a CDR-L1 comprising the amino acid sequence set forth in any one of SEQ ID NOs: 300-339, 420-434, 553-577, 670-683, and 744-751. In some embodiments, the pharmaceutical composition comprises an anti-CD30L antibody or antigen binding fragment, wherein the antibody or antigen binding fragment thereof comprises a CDR-H2 comprising the amino acid sequence set forth in any one of SEQ ID NOs: 140-179, 235-249, 490-499, 513-527, 642-655, and 724-735 and a CDR-L2 comprising the amino acid sequence set forth in any one of SEQ ID NOs: 340-379, 435-449, 578-602, 684-697, and 752-759. In certain embodiments, the pharmaceutical composition comprises an anti-CD30L antibody or antigen binding fragment, wherein the antibody or antigen binding fragment thereof comprises a CDR-H2 comprising the amino acid sequence set forth in any one of SEQ ID NOs: 140-179, 235-249, 490-499, 513-527, 642-655, and 724-735 and a CDR-L3 comprising the amino acid sequence set forth in any one of SEQ ID NOs: 380-419, 450-464, 603-627, 698-711, and 760-765. In other embodiments, the pharmaceutical composition comprises an anti-CD30L antibody or antigen binding fragment, wherein the antibody or antigen binding fragment thereof comprises a CDR-H3 comprising the amino acid sequence set forth in any one of SEQ Id NOs: 180-219, 250-264, 528-552, 656-669, and 736-743 and a CDR-L1 comprising the amino acid sequence set forth in any one of SEQ ID NOs: 300-339, 420-434, 553-577, 670-683, and 744-751. In yet other embodiments, the pharmaceutical composition comprises an anti-CD30L antibody or antigen binding fragment, wherein the antibody or antigen binding fragment thereof comprises a CDR-H3 comprising the amino acid sequence set forth in any one of SEQ Id NOs: 180-219, 250-264, 528-552, 656-669, and 736-743 and a CDR-L2 comprising the amino acid sequence set forth in any one of SEQ ID NOs: 340-379, 435-449, 578-602, 684-697, and 752-759. In some embodiments, the pharmaceutical composition comprises an anti-CD30L antibody or antigen binding fragment, wherein the antibody or antigen binding fragment thereof comprises a CDR-H3 comprising the amino acid sequence set forth in any one of SEQ Id NOs: 180-219, 250-264, 528-552, 656-669, and 736-743 and a CDR-L3 comprising the amino acid sequence set forth in any one of SEQ ID NOs: 380-419, 450-464, 603-627, 698-711, and 760-765. In one embodiment, the pharmaceutical composition comprises an anti-CD30L antibody or antigen binding fragment, wherein the antibody or antigen binding fragment thereof comprises a CDR-L1 comprising the amino acid sequence set forth in any one of SEQ ID NOs: 300-339, 420-434, 553-577, 670-683, and 744-751 and a CDR-L2 comprising the amino acid sequence set forth in any one of SEQ ID NOs: 340-379, 435-449, 578-602, 684-697, and 752-759. In another embodiment, the pharmaceutical composition comprises an anti-CD30L antibody or antigen binding fragment, wherein the antibody or antigen binding fragment thereof comprises a CDR-L1 comprising the amino acid sequence set forth in any one of SEQ ID NOs: 300-339, 420-434, 553-577, 670-683, and 744-751 and a CDR-L3 comprising the amino acid sequence set forth in any one of SEQ ID NOs: 380-419, 450-464, 603-627, 698-711, and 760-765. In another embodiment, the pharmaceutical composition comprises an anti-CD30L antibody or antigen binding fragment, wherein the antibody or antigen binding fragment thereof comprises a CDR-L2 comprising the amino acid sequence set forth in any one of SEQ ID NOs: 340-379, 435-449, 578-602, 684-697, and 752-759 and a CDR-L3 comprising the amino acid sequence set forth in any one of SEQ ID NOs: 380-419, 450-464, 603-627, 698-711, and 760-765. In other embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, and a CDR-H3 comprising the amino acid sequence set forth in any one of SEQ Id NOs: 180-219, 250-264, 528-552, 656-669, and 736-743. In yet other embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735 and a CDR-L1 comprising the amino acid sequence set forth in any one of SEQ ID NOs: 300-339, 420-434, 553-577, 670-683, and 744-751. In further embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, and a CDR-L2 comprising the amino acid sequence set forth in any one of SEQ ID NOs: 340-379, 435-449, 578-602, 684-697, and 752-759. In some embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, and a CDR-L3 comprising the amino acid sequence set forth in any one of SEQ ID NOs: 380-419, 450-464, 603-627, 698-711, and 760-765. In some embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723, a CDR-H3 comprising the amino acid sequence set forth in any one of SEQ Id NOs: 180-219, 250-264, 528-552, 656-669, and 736-743 and a CDR-L1 comprising the amino acid sequence set forth in any one of SEQ ID NOs: 300-339, 420-434, 553-577, 670-683, and 744-751. In certain embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723, a CDR-H3 comprising the amino acid sequence set forth in any one of SEQ Id NOs: 180-219, 250-264, 528-552, 656-669, and 736-743, and a CDR-L2 comprising the amino acid sequence set forth in any one of SEQ ID NOs: 340-379, 435-449, 578-602, 684-697, and 752-759. In some embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723, a CDR-H3 comprising the amino acid sequence set forth in any one of SEQ Id NOs: 180-219, 250-264, 528-552, 656-669, and 736-743, and a CDR-L3 comprising the amino acid sequence set forth in any one of SEQ ID NOs: 380-419, 450-464, 603-627, 698-711, and 760-765. In yet other embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723, a CDR-L1 comprising the amino acid sequence set forth in any one of SEQ ID NOs: 300-339, 420-434, 553-577, 670-683, and 744-751 and a CDR-L2 comprising the amino acid sequence set forth in any one of SEQ ID NOs: 340-379, 435-449, 578-602, 684-697, and 752-759. In some embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723, a CDR-L1 comprising the amino acid sequence set forth in any one of SEQ ID NOs: 300-339, 420-434, 553-577, 670-683, and 744-751 and a CDR-L3 comprising the amino acid sequence set forth in any one of SEQ ID NOs: 380-419, 450-464, 603-627, 698-711, and 760-765. In some embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723, a CDR-L2 comprising the amino acid sequence set forth in any one of SEQ ID NOs: 340-379, 435-449, 578-602, 684-697, and 752-759 and a CDR-L3 comprising the amino acid sequence set forth in any one of SEQ ID NOs: 380-419, 450-464, 603-627, 698-711, and 760-765. In some embodiments, the pharmaceutical composition comprises an anti-CD30L antibody or antigen binding fragment, wherein the antibody or antigen binding fragment thereof comprises a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743 and a CDR-L1 comprising the amino acid sequence set forth in any one of SEQ ID NOs: 300-339, 420-434, 553-577, 670-683, and 744-751. In certain embodiments, the pharmaceutical composition comprises an anti-CD30L antibody or antigen binding fragment, wherein the antibody or antigen binding fragment thereof comprises a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, and a CDR-L2 comprising the amino acid sequence set forth in any one of SEQ ID NOs: 340-379, 435-449, 578-602, 684-697, and 752-759. In some embodiments, the pharmaceutical composition comprises an anti-CD30L antibody or antigen binding fragment, wherein the antibody or antigen binding fragment thereof comprises a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, and a CDR-L3 comprising the amino acid sequence set forth in any one of SEQ ID NOs: 380-419, 450-464, 603-627, 698-711, and 760-765. In other embodiments, the pharmaceutical composition comprises an anti-CD30L antibody or antigen binding fragment, wherein the antibody or antigen binding fragment thereof comprises a CDR-H2 comprising the amino acid sequence set forth in any one of SEQ ID NOs: 140-179, 235-249, 490-499, 513-527, 642-655, and 724-735, a CDR-L1 comprising the amino acid sequence set forth in any one of SEQ ID NOs: 300-339, 420-434, 553-577, 670-683, and 744-751 and a CDR-L2 comprising the amino acid sequence set forth in any one of SEQ ID NOs: 340-379, 435-449, 578-602, 684-697, and 752-759. In yet other embodiments, the pharmaceutical composition comprises an anti-CD30L antibody or antigen binding fragment, wherein the antibody or antigen binding fragment thereof comprises a CDR-H2 comprising the amino acid sequence set forth in any one of SEQ ID NOs: 140-179, 235-249, 490-499, 513-527, 642-655, and 724-735, a CDR-L1 comprising the amino acid sequence set forth in any one of SEQ ID NOs: 300-339, 420-434, 553-577, 670-683, and 744-751 and a CDR-L3 comprising the amino acid sequence set forth in any one of SEQ ID NOs: 380-419, 450-464, 603-627, 698-711, and 760-765. In some embodiments, the pharmaceutical composition comprises an anti-CD30L antibody or antigen binding fragment, wherein the antibody or antigen binding fragment thereof comprises a CDR-H2 comprising the amino acid sequence set forth in any one of SEQ ID NOs: 140-179, 235-249, 490-499, 513-527, 642-655, and 724-735, a CDR-L2 comprising the amino acid sequence set forth in any one of SEQ ID NOs: 340-379, 435-449, 578-602, 684-697, and 752-759 and a CDR-L3 comprising the amino acid sequence set forth in any one of SEQ ID NOs: 380-419, 450-464, 603-627, 698-711, and 760-765. In yet other embodiments, the pharmaceutical composition comprises an anti-CD30L antibody or antigen binding fragment, wherein the antibody or antigen binding fragment thereof comprises a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751 and a CDR-L2 comprising the amino acid sequence set forth in any one of SEQ ID NOs: 340-379, 435-449, 578-602, 684-697, and 752-759. In other embodiments, the pharmaceutical composition comprises an anti-CD30L antibody or antigen binding fragment, wherein the antibody or antigen binding fragment thereof comprises a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751 and a CDR-L3 comprising the amino acid sequence set forth in any one of SEQ ID NOs: 380-419, 450-464, 603-627, 698-711, and 760-765. In certain embodiments, the pharmaceutical composition comprises an anti-CD30L antibody or antigen binding fragment, wherein the antibody or antigen binding fragment thereof comprises a CDR-H3 comprising the amino acid sequence set forth in any one of SEQ Id NOs: 180-219, 250-264, 528-552, 656-669, and 736-743, a CDR-L2 comprising the amino acid sequence set forth in any one of SEQ ID NOs: 340-379, 435-449, 578-602, 684-697, and 752-759 and a CDR-L3 comprising the amino acid sequence set forth in any one of SEQ ID NOs: 380-419, 450-464, 603-627, 698-711, and 760-765. In some embodiments, the pharmaceutical composition comprises an anti-CD30L antibody or antigen binding fragment, wherein the antibody or antigen binding fragment thereof comprises a CDR-L1 comprising the amino acid sequence set forth in any one of SEQ ID NOs: 300-339, 420-434, 553-577, 670-683, and 744-751, a CDR-L2 comprising the amino acid sequence set forth in any one of SEQ ID NOs: 340-379, 435-449, 578-602, 684-697, and 752-759 and a CDR-L3 comprising the amino acid sequence set forth in any one of SEQ ID NOs: 380-419, 450-464, 603-627, 698-711, and 760-765. In other embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, and a CDR-L1 comprising the amino acid sequence set forth in any one of SEQ ID NOs: 300-339, 420-434, 553-577, 670-683, and 744-751. In further embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, and a CDR-L2 comprising the amino acid sequence set forth in any one of SEQ ID NOs: 340-379, 435-449, 578-602, 684-697, and 752-759. In some embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, and a CDR-L3 comprising the amino acid sequence set forth in any one of SEQ ID NOs: 380-419, 450-464, 603-627, 698-711, and 760-765. In some embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-L1 comprising the amino acid sequence set forth in any one of SEQ ID NOs: 300-339, 420-434, 553-577, 670-683, and 744-751, and a CDR-L2 comprising the amino acid sequence set forth in any one of SEQ ID NOs: 340-379, 435-449, 578-602, 684-697, and 752-759. In some embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-L1 comprising the amino acid sequence set forth in any one of SEQ ID NOs: 300-339, 420-434, 553-577, 670-683, and 744-751, and a CDR-L3 comprising the amino acid sequence set forth in any one of SEQ ID NOs: 380-419, 450-464, 603-627, 698-711, and 760-765. In some embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765. In certain embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, and a CDR-L2 comprising the amino acid sequence set forth in any one of SEQ ID NOs: 340-379, 435-449, 578-602, 684-697, and 752-759. In certain embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, and a CDR-L3 comprising the amino acid sequence set forth in any one of SEQ ID NOs: 380-419, 450-464, 603-627, 698-711, and 760-765. In certain embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723, a CDR-H3 comprising the amino acid sequence set forth in any one of SEQ Id NOs: 180-219, 250-264, 528-552, 656-669, and 736-743, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765. In yet other embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723, a CDR-L1 comprising the amino acid sequence set forth in any one of SEQ ID NOs: 300-339, 420-434, 553-577, 670-683, and 744-751, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765. In further embodiments, the pharmaceutical composition comprises an anti-CD30L antibody or antigen binding fragment, wherein the antibody or antigen binding fragment thereof comprises a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, and a CDR-L2 comprising the amino acid sequence set forth in any one of SEQ ID NOs: 340-379, 435-449, 578-602, 684-697, and 752-759. In some embodiments, the pharmaceutical composition comprises an anti-CD30L antibody or antigen binding fragment, wherein the antibody or antigen binding fragment thereof comprises a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, and a CDR-L3 comprising the amino acid sequence set forth in any one of SEQ ID NOs: 380-419, 450-464, 603-627, 698-711, and 760-765. In further embodiments, the pharmaceutical composition comprises an anti-CD30L antibody or antigen binding fragment, wherein the antibody or antigen binding fragment thereof comprises a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765. In some embodiments, the pharmaceutical composition comprises an anti-CD30L antibody or antigen binding fragment, wherein the antibody or antigen binding fragment thereof comprises a CDR-H2 comprising the amino acid sequence set forth in any one of SEQ ID NOs: 140-179, 235-249, 490-499, 513-527, 642-655, and 724-735, a CDR-L1 comprising the amino acid sequence set forth in any one of SEQ ID NOs: 300-339, 420-434, 553-577, 670-683, and 744-751, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765. In some embodiments, the pharmaceutical composition comprises an anti-CD30L antibody or antigen binding fragment, wherein the antibody or antigen binding fragment thereof comprises a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765. In certain embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, and a CDR-L2 comprising the amino acid sequence set forth in any one of SEQ ID NOs: 340-379, 435-449, 578-602, 684-697, and 752-759. In some embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, and a CDR-L3 comprising the amino acid sequence set forth in any one of SEQ ID NOs: 380-419, 450-464, 603-627, 698-711, and 760-765. In some embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765. In certain embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-L1 comprising the amino acid sequence set forth in any one of SEQ ID NOs: 300-339, 420-434, 553-577, 670-683, and 744-751, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765. In certain embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765. In some embodiments, the pharmaceutical composition comprises an anti-CD30L antibody or antigen binding fragment, wherein the antibody or antigen binding fragment thereof comprises a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765. In some embodiments, the pharmaceutical composition comprises an anti-CD30L antibody or antigen binding fragment, wherein the antibody or antigen binding fragment thereof comprises a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765. In some embodiments, the pharmaceutical composition comprises an anti-CD30L antibody or antigen binding fragment, wherein the antibody or antigen binding fragment thereof comprises any one of a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765. In some embodiments, the pharmaceutical composition comprises an anti-CD30L antibody or antigen binding fragment, wherein the antibody or antigen binding fragment thereof comprises any two of a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765, in any combination or permutation. In some embodiments, the pharmaceutical composition comprises an anti-CD30L antibody or antigen binding fragment, wherein the antibody or antigen binding fragment thereof comprises any three of a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765, in any combination or permutation. In some embodiments, the pharmaceutical composition comprises an anti-CD30L antibody or antigen binding fragment, wherein the antibody or antigen binding fragment thereof comprises any four of a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765, in any combination or permutation. In some embodiments, the pharmaceutical composition comprises an anti-CD30L antibody or antigen binding fragment, wherein the antibody or antigen binding fragment thereof comprises any five of a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765, in any combination or permutation. In some embodiments, the pharmaceutical composition comprises an anti-CD30L antibody or antigen binding fragment, wherein the antibody or antigen binding fragment thereof comprises all six of a CDR-H1 comprising the amino acid sequence set forth in any one of SEQ ID NOs: 100-139, 220-234, 465-489, 628-641, and 712-723, a CDR-H2 comprising the amino acid sequence set forth in any one of SEQ ID NOs: 140-179, 235-249, 490-499, 513-527, 642-655, and 724-735, a CDR-H3 comprising the amino acid sequence set forth in any one of SEQ Id NOs: 180-219, 250-264, 528-552, 656-669, and 736-743, a CDR-L1 comprising the amino acid sequence set forth in any one of SEQ ID NOs: 300-339, 420-434, 553-577, 670-683, and 744-751, a CDR-L2 comprising the amino acid sequence set forth in any one of SEQ ID NOs: 340-379, 435-449, 578-602, 684-697, and 752-759, and a CDR-L3 comprising the amino acid sequence set forth in any one of SEQ ID NOs: 380-419, 450-464, 603-627, 698-711, and 760-765.

In some embodiments of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in any one of SEQ ID NOs: 100-109, 628, 635, and 712-723; (b) a CDR-H2 comprising the amino acid sequence set forth in any one of SEQ ID NOs: 140-149, 642, 649, and 724-735; (c) a CDR-H3 comprising the amino acid sequence set forth in any one of SEQ ID NOs: 180-189, 656, 663, and 736-743; (d) a CDR-L1 comprising the amino acid sequence set forth in any one of SEQ ID NOs: 300-309, 670, 677, and 744-751; (e) a CDR-L2 comprising the amino acid sequence set forth in any one of SEQ ID NOs: 340-349, 684, 691, and 752-759; and/or (f) a CDR-L3 comprising the amino acid sequence set forth in any one of SEQ ID Nos: 380-389, 698, 705, and 760-765.

In certain embodiments of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in any one of SEQ ID NOs: 100-109, 628, 635, and 712-723; (b) a CDR-H2 comprising the amino acid sequence set forth in any one of SEQ ID NOs: 140-149, 642, 649, and 724-735; (c) a CDR-H3 comprising the amino acid sequence set forth in any one of SEQ ID NOs: 180-189, 656, 663, and 736-743; (d) a CDR-L1 comprising the amino acid sequence set forth in any one of SEQ ID NOs: 300-309, 670, 677, and 744-751; (e) a CDR-L2 comprising the amino acid sequence set forth in any one of SEQ ID NOs: 340-349, 684, 691, and 752-759; and/or (f) a CDR-L3 comprising the amino acid sequence set forth in any one of SEQ ID Nos: 380-389, 698, 705, and 760-765.

In certain embodiments of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in any one of SEQ ID NOs: 105-109, 628, 635, and 712-723; (b) a CDR-H2 comprising the amino acid sequence set forth in any one of SEQ ID NOs: 145-149, 642, 649, and 724-735; (c) a CDR-H3 comprising the amino acid sequence set forth in any one of SEQ ID NOs: 185-189, 656, 663, and 736-743; (d) a CDR-L1 comprising the amino acid sequence set forth in any one of SEQ ID NOs: 305-309, 670, 677, and 744-751; (e) a CDR-L2 comprising the amino acid sequence set forth in any one of SEQ ID NOs: 345-349, 684, 691, and 752-759; and/or (f) a CDR-L3 comprising the amino acid sequence set forth in any one of SEQ ID NOs: 385-389, 698, 705, and 760-765.

In one aspect, provided herein is a pharmaceutical composition comprising an antibody or antigen binding fragment thereof that binds CD30L, wherein the antibody or antigen binding fragment thereof binds to an epitope comprising one or more amino acids in CD30L selected from the group consisting of N165, K166, I168, K169, and D234, wherein the amino acids are numbered according to the amino acid sequence of CD30L as set forth in SEQ ID NO:33.

In another aspect, provided herein is a pharmaceutical composition comprising an antibody or antigen binding fragment thereof that binds CD30L, wherein the antibody or antigen binding fragment thereof binds to an epitope comprising one or more amino acids in CD30L selected from the group consisting of H167, S217, and D118, wherein the amino acids are numbered according to the amino acid sequence of CD30L as set forth in SEQ ID NO: 33.

In yet another aspect, provided herein is a pharmaceutical composition comprising an antibody or antigen binding fragment thereof that binds CD30L, wherein the antibody or antigen binding fragment thereof binds to an epitope comprising one or more amino acids in CD30L selected from the group consisting of D118, N165, K166, H167, I168, K169, S217, and D234, wherein the amino acids are numbered according to the amino acid sequence of CD30L as set forth in SEQ ID NO:33.

The anti-CD30L antibody in the pharmaceutical composition provided herein can bind to epitope of various combinations of amino acids on CD30L as described herein. Accordingly, in one embodiment of the pharmaceutical composition comprising an anti-CD30L antibody or antigen binding fragment provided herein, including in Section 2 and this Section (Section 4.5), the antibody or antigen binding fragment thereof binds to an epitope comprising any one amino acid in CD30L selected from the group consisting of N165, K166, I168, K169, and D234. In another embodiment of the pharmaceutical composition comprising an anti-CD30L antibody or antigen binding fragment provided herein, including in Section 2 and this Section (Section 4.5), the antibody or antigen binding fragment thereof binds to an epitope comprising any two amino acids in CD30L selected from the group consisting of N165, K166, I168, K169, and D234. In a further embodiment of the pharmaceutical composition comprising an anti-CD30L antibody or antigen binding fragment provided herein, including in Section 2 and this Section (Section 4.5), the antibody or antigen binding fragment thereof binds to an epitope comprising any three amino acids in CD30L selected from the group consisting of N165, K166, I168, K169, and D234. In yet another embodiment of the pharmaceutical composition comprising an anti-CD30L antibody or antigen binding fragment provided herein, including in Section 2 and this Section (Section 4.5), the antibody or antigen binding fragment thereof binds to an epitope comprising any four amino acids in CD30L selected from the group consisting of N165, K166, I168, K169, and D234. In a further embodiment of the pharmaceutical composition comprising an anti-CD30L antibody or antigen binding fragment provided herein, including in Section 2 and this Section (Section 4.5), the antibody or antigen binding fragment thereof binds to an epitope comprising any five amino acids in CD30L selected from the group consisting of N165, K166, I168, K169, and D234. The position of the amino acids in this paragraph are numbered according to the amino acid sequence of CD30L as set forth in SEQ ID NO:33.

Additionally, the anti-CD30L antibody in the pharmaceutical composition provided herein can bind to various combinations of amino acids on CD30L in addition to the epitope described in the previous paragraph. As such, in one embodiment of the pharmaceutical composition comprising an anti-CD30L antibody or antigen binding fragment provided herein, including in Section 2 and this Section (Section 4.5), the antibody or antigen binding fragment thereof binds to an epitope further comprising any one amino acid in CD30L selected from the group consisting of H167, S217, and D118. In another embodiment of the pharmaceutical composition comprising an anti-CD30L antibody or antigen binding fragment provided herein, including in Section 2 and this Section (Section 4.5), the antibody or antigen binding fragment thereof binds to an epitope further comprising any two amino acids in CD30L selected from the group consisting of H167, S217, and D118. In yet another embodiment of the pharmaceutical composition comprising an anti-CD30L antibody or antigen binding fragment provided herein, including in Section 2 and this Section (Section 4.5), the antibody or antigen binding fragment thereof binds to an epitope further comprising three amino acids in CD30L selected from the group consisting of H167, S217, and D118. Alternatively, the anti-CD30L antibody or antigen binding fragment in the pharmaceutical composition provided herein can bind to various other combinations of amino acids on CD30L. In one embodiment of the pharmaceutical composition comprising an anti-CD30L antibody or antigen binding fragment provided herein, including in Section 2 and this Section (Section 4.5), the antibody or antigen binding fragment thereof binds to an epitope comprising any one amino acid in CD30L selected from the group consisting of H167, S217, and D118. In another embodiment of the pharmaceutical composition comprising an anti-CD30L antibody or antigen binding fragment provided herein, including in Section 2 and this Section (Section 4.5), the antibody or antigen binding fragment thereof binds to an epitope comprising any two amino acids in CD30L selected from the group consisting of H167, S217, and D118. In yet another embodiment of the pharmaceutical composition comprising an anti-CD30L antibody or antigen binding fragment provided herein, including in Section 2 and this Section (Section 4.5), the antibody or antigen binding fragment thereof binds to an epitope comprising three amino acids in CD30L selected from the group consisting of H167, S217, and D118. The position of the amino acids in this paragraph are numbered according to the amino acid sequence of CD30L as set forth in SEQ ID NO:33.

Similarly, the anti-CD30L antibody in the pharmaceutical composition provided herein can bind to epitope of various other combinations of amino acids on CD30L as described herein. Accordingly, in one embodiment of the pharmaceutical composition comprising an anti-CD30L antibody or antigen binding fragment provided herein, including in Section 2 and this Section (Section 4.5), the antibody or antigen binding fragment thereof binds to an epitope comprising any one amino acid in CD30L selected from the group consisting of D118, N165, K166, H167, I168, K169, S217, and D234. In another embodiment of the pharmaceutical composition comprising an anti-CD30L antibody or antigen binding fragment provided herein, including in Section 2 and this Section (Section 4.5), the antibody or antigen binding fragment thereof binds to an epitope comprising any two amino acids in CD30L selected from the group consisting of D118, N165, K166, H167, I168, K169, S217, and D234. In a further embodiment of the pharmaceutical composition comprising an anti-CD30L antibody or antigen binding fragment provided herein, including in Section 2 and this Section (Section 4.5), the antibody or antigen binding fragment thereof binds to an epitope comprising any three amino acids in CD30L selected from the group consisting of D118, N165, K166, H167, I168, K169, S217, and D234. In yet another embodiment of the pharmaceutical composition comprising an anti-CD30L antibody or antigen binding fragment provided herein, including in Section 2 and this Section (Section 4.5), the antibody or antigen binding fragment thereof binds to an epitope comprising any four amino acids in CD30L selected from the group consisting of D118, N165, K166, H167, I168, K169, S217, and D234. In a further embodiment of the pharmaceutical composition comprising an anti-CD30L antibody or antigen binding fragment provided herein, including in Section 2 and this Section (Section 4.5), the antibody or antigen binding fragment thereof binds to an epitope comprising any five amino acids in CD30L selected from the group consisting of D118, N165, K166, H167, I168, K169, S217, and D234. In one embodiment of the pharmaceutical composition comprising an anti-CD30L antibody or antigen binding fragment provided herein, including in Section 2 and this Section (Section 4.5), the antibody or antigen binding fragment thereof binds to an epitope comprising any six amino acids in CD30L selected from the group consisting of D118, N165, K166, H167, I168, K169, S217, and D234. In another embodiment of the pharmaceutical composition comprising an anti-CD30L antibody or antigen binding fragment provided herein, including in Section 2 and this Section (Section 4.5), the antibody or antigen binding fragment thereof binds to an epitope comprising any seven amino acids in CD30L selected from the group consisting of D118, N165, K166, H167, I168, K169, S217, and D234. In a further embodiment of the pharmaceutical composition comprising an anti-CD30L antibody or antigen binding fragment provided herein, including in Section 2 and this Section (Section 4.5), the antibody or antigen binding fragment thereof binds to an epitope comprising eight amino acids in CD30L selected from the group consisting of D118, N165, K166, H167, I168, K169, S217, and D234. The position of the amino acids in this paragraph are numbered according to the amino acid sequence of CD30L as set forth in SEQ ID NO:33.

Furthermore, the anti-CD30L antibody in the pharmaceutical composition provided herein including in Section 2 and this Section (Section 4.5) can bind to certain specific epitope of specific combinations of amino acids on CD30L as described herein and such epitopes are further provided in this paragraph. Accordingly, in one embodiment, the epitope for the anti-CD30L in the pharmaceutical composition provided herein comprises or consists of N165. In some embodiments, the epitope for the anti-CD30L in the pharmaceutical composition provided herein comprises or consists of K166. In certain embodiments, the epitope for the anti-CD30L in the pharmaceutical composition provided herein comprises or consists of I168. In other embodiments, the epitope for the anti-CD30L in the pharmaceutical composition provided herein comprises or consists of K169. In yet other embodiments, the epitope for the anti-CD30L in the pharmaceutical composition provided herein comprises or consists of D234. In another embodiment, the epitope for the anti-CD30L in the pharmaceutical composition provided herein comprises or consists of H167. In yet another embodiment, the epitope for the anti-CD30L in the pharmaceutical composition provided herein comprises or consists of S217. In yet another embodiment, the epitope for the anti-CD30L in the pharmaceutical composition provided herein comprises or consists of D118. In one embodiment, the epitope for the anti-CD30L in the pharmaceutical composition provided herein comprises or consists of N165 and K166. In some embodiments, the epitope for the anti-CD30L in the pharmaceutical composition provided herein comprises or consists of N165 and I168. In certain embodiments, the epitope for the anti-CD30L in the pharmaceutical composition provided herein comprises or consists of N165 and K169. In other embodiments, the epitope for the anti-CD30L in the pharmaceutical composition provided herein comprises or consists of N165 and D234. In some embodiments, the epitope for the anti-CD30L in the pharmaceutical composition provided herein comprises or consists of N165 and H167. In other embodiments, the epitope for the anti-CD30L in the pharmaceutical composition provided herein comprises or consists of N165 and S217. In further embodiments, the epitope for the anti-CD30L in the pharmaceutical composition provided herein comprises or consists of K166 and I168. In one embodiment, the epitope for the anti-CD30L in the pharmaceutical composition provided herein comprises or consists of K166 and K169. In some embodiments, the epitope for the anti-CD30L in the pharmaceutical composition provided herein comprises or consists of K166 and D234. In certain embodiments, the epitope for the anti-CD30L in the pharmaceutical composition provided herein comprises or consists of K166 and H167. In some embodiments, the epitope for the anti-CD30L in the pharmaceutical composition provided herein comprises or consists of K166 and S217. In other embodiments, the epitope for the anti-CD30L in the pharmaceutical composition provided herein comprises or consists of I168 and K169. In yet other embodiments, the epitope for the anti-CD30L in the pharmaceutical composition provided herein comprises or consists of I168 and D234. In some embodiments, the epitope for the anti-CD30L in the pharmaceutical composition provided herein comprises or consists of I168 and H167. In certain embodiments, the epitope for the anti-CD30L in the pharmaceutical composition provided herein comprises or consists of I168 and S217. In one embodiment, the epitope for the anti-CD30L in the pharmaceutical composition provided herein comprises or consists of K169 and D234. In another embodiment, the epitope for the anti-CD30L in the pharmaceutical composition provided herein comprises or consists of K169 and H167. In one embodiment, the epitope for the anti-CD30L in the pharmaceutical composition provided herein comprises or consists of K169 and S217. In some embodiments, the epitope for the anti-CD30L in the pharmaceutical composition provided herein comprises or consists of D234 and H167. In certain embodiments, the epitope for the anti-CD30L in the pharmaceutical composition provided herein comprises or consists of D234 and S217. In one embodiment, the epitope for the anti-CD30L in the pharmaceutical composition provided herein comprises or consists of H167 and S217. In one embodiment, the epitope for the anti-CD30L in the pharmaceutical composition provided herein comprises or consists of N165 and D118. In another embodiment, the epitope for the anti-CD30L in the pharmaceutical composition provided herein comprises or consists of K166 and D118. In some embodiments, the epitope for the anti-CD30L in the pharmaceutical composition provided herein comprises or consists of I168 and D118. In certain embodiments, the epitope for the anti-CD30L in the pharmaceutical composition provided herein comprises or consists of K169 and D118. In other embodiments, the epitope for the anti-CD30L in the pharmaceutical composition provided herein comprises or consists of D234 and D118. In some embodiments, the epitope for the anti-CD30L in the pharmaceutical composition provided herein comprises or consists of H167 and D118. In other embodiments, the epitope for the anti-CD30L in the pharmaceutical composition provided herein comprises or consists of S217 and D118. In other embodiments, the epitope for the anti-CD30L in the pharmaceutical composition provided herein comprises or consists of N165, K166, and I168. In yet other embodiments, the epitope for the anti-CD30L in the pharmaceutical composition provided herein comprises or consists of N165, K166 and K169. In further embodiments, the epitope for the anti-CD30L in the pharmaceutical composition provided herein comprises or consists of N165, K166, and D234. In some embodiments, the epitope for the anti-CD30L in the pharmaceutical composition provided herein comprises or consists of N165, K166, and H167. In certain embodiments, the epitope for the anti-CD30L in the pharmaceutical composition provided herein comprises or consists of N165, K166, and S217. In some embodiments, the epitope for the anti-CD30L in the pharmaceutical composition provided herein comprises or consists of N165, I168 and K169. In certain embodiments, the epitope for the anti-CD30L in the pharmaceutical composition provided herein comprises or consists of N165, I168, and D234. In some embodiments, the epitope for the anti-CD30L in the pharmaceutical composition provided herein comprises or consists of N165, I168, and H167. In other embodiments, the epitope for the anti-CD30L in the pharmaceutical composition provided herein comprises or consists of N165, I168, and S217. In yet other embodiments, the epitope for the anti-CD30L in the pharmaceutical composition provided herein comprises or consists of N165, K169 and D234. In some embodiments, the epitope for the anti-CD30L in the pharmaceutical composition provided herein comprises or consists of N165, K169 and H167. In other embodiments, the epitope for the anti-CD30L in the pharmaceutical composition provided herein comprises or consists of N165, K169 and S217. In some embodiments, the epitope for the anti-CD30L in the pharmaceutical composition provided herein comprises or consists of N165, D234 and H167. In certain embodiments, the epitope for the anti-CD30L in the pharmaceutical composition provided herein comprises or consists of N165, D234 and S217. In other embodiments, the epitope for the anti-CD30L in the pharmaceutical composition provided herein comprises or consists of N165, H167 and S217. In some embodiments, the epitope for the anti-CD30L in the pharmaceutical composition provided herein comprises or consists of K166, I168 and K169. In certain embodiments, the epitope for the anti-CD30L in the pharmaceutical composition provided herein comprises or consists of K166, I168, and D234. In some embodiments, the epitope for the anti-CD30L in the pharmaceutical composition provided herein comprises or consists of K166, I168, and H167. In certain embodiments, the epitope for the anti-CD30L in the pharmaceutical composition provided herein comprises or consists of K166, I168, and S217. In other embodiments, the epitope for the anti-CD30L in the pharmaceutical composition provided herein comprises or consists of K166, K169 and D234. In yet other embodiments, the epitope for the anti-CD30L in the pharmaceutical composition provided herein comprises or consists of K166, K169 and H167. In some embodiments, the epitope for the anti-CD30L in the pharmaceutical composition provided herein comprises or consists of K166, K169 and S217. In some embodiments, the epitope for the anti-CD30L in the pharmaceutical composition provided herein comprises or consists of K166, D234 and H167. In some embodiments, the epitope for the anti-CD30L in the pharmaceutical composition provided herein comprises or consists of K166, D234 and S217. In some embodiments, the epitope for the anti-CD30L in the pharmaceutical composition provided herein comprises or consists of K166, H167 and S217. In yet other embodiments, the epitope for the anti-CD30L in the pharmaceutical composition provided herein comprises or consists of I168, K169 and D234. In other embodiments, the epitope for the anti-CD30L in the pharmaceutical composition provided herein comprises or consists of I168, K169 and H167. In some embodiments, the epitope for the anti-CD30L in the pharmaceutical composition provided herein comprises or consists of I168, K169 and S217. In certain embodiments, the epitope for the anti-CD30L in the pharmaceutical composition provided herein comprises or consists of I168, D234 and H167. In other embodiments, the epitope for the anti-CD30L in the pharmaceutical composition provided herein comprises or consists of I168, D234 and S217. In yet other embodiments, the epitope for the anti-CD30L in the pharmaceutical composition provided herein comprises or consists of I168, H167 and S217. In some embodiments, the epitope for the anti-CD30L in the pharmaceutical composition provided herein comprises or consists of K169, D234 and H167. In some embodiments, the epitope for the anti-CD30L in the pharmaceutical composition provided herein comprises or consists of K169, D234 and S217. In some embodiments, the epitope for the anti-CD30L in the pharmaceutical composition provided herein comprises or consists of K169, H167 and S217. In some embodiments, the epitope for the anti-CD30L in the pharmaceutical composition provided herein comprises or consists of D234, H167 and S217. In one embodiment, the epitope for the anti-CD30L in the pharmaceutical composition provided herein comprises or consists of N165, K166, and D118. In some embodiments, the epitope for the anti-CD30L in the pharmaceutical composition provided herein comprises or consists of N165, I168, and D118. In certain embodiments, the epitope for the anti-CD30L in the pharmaceutical composition provided herein comprises or consists of N165, K169, and D118. In other embodiments, the epitope for the anti-CD30L in the pharmaceutical composition provided herein comprises or consists of N165, D234, and D118. In some embodiments, the epitope for the anti-CD30L in the pharmaceutical composition provided herein comprises or consists of N165, H167, and D118. In other embodiments, the epitope for the anti-CD30L in the pharmaceutical composition provided herein comprises or consists of N165, S217, and D118. In further embodiments, the epitope for the anti-CD30L in the pharmaceutical composition provided herein comprises or consists of K166, I168, and D118. In one embodiment, the epitope for the anti-CD30L in the pharmaceutical composition provided herein comprises or consists of K166, K169, and D118. In some embodiments, the epitope for the anti-CD30L in the pharmaceutical composition provided herein comprises or consists of K166, D234, and D118. In certain embodiments, the epitope for the anti-CD30L in the pharmaceutical composition provided herein comprises or consists of K166, H167, and D118. In some embodiments, the epitope for the anti-CD30L in the pharmaceutical composition provided herein comprises or consists of K166, S217, and D118. In other embodiments, the epitope for the anti-CD30L in the pharmaceutical composition provided herein comprises or consists of I168, K169, and D118. In yet other embodiments, the epitope for the anti-CD30L in the pharmaceutical composition provided herein comprises or consists of I168, D234, and D118. In some embodiments, the epitope for the anti-CD30L in the pharmaceutical composition provided herein comprises or consists of I168, H167, and D118. In certain embodiments, the epitope for the anti-CD30L in the pharmaceutical composition provided herein comprises or consists of I168, S217, and D118. In one embodiment, the epitope for the anti-CD30L in the pharmaceutical composition provided herein comprises or consists of K169, D234, and D118. In another embodiment, the epitope for the anti-CD30L in the pharmaceutical composition provided herein comprises or consists of K169, H167, and D118. In one embodiment, the epitope for the anti-CD30L in the pharmaceutical composition provided herein comprises or consists of K169, S217, and D118. In some embodiments, the epitope for the anti-CD30L in the pharmaceutical composition provided herein comprises or consists of D234, H167, and D118. In certain embodiments, the epitope for the anti-CD30L in the pharmaceutical composition provided herein comprises or consists of D234, S217, and D118. In one embodiment, the epitope for the anti-CD30L in the pharmaceutical composition provided herein comprises or consists of H167, S217, and D118. In other embodiments, the epitope for the anti-CD30L in the pharmaceutical composition provided herein comprises or consists of N165, K166, I168, and K169. In further embodiments, the epitope for the anti-CD30L in the pharmaceutical composition provided herein comprises or consists of N165, K166, I168, and D234. In some embodiments, the epitope for the anti-CD30L in the pharmaceutical composition provided herein comprises or consists of N165, K166, I168, and H167. In other embodiments, the epitope for the anti-CD30L in the pharmaceutical composition provided herein comprises or consists of N165, K166, I168, and S217. In some embodiments, the epitope for the anti-CD30L in the pharmaceutical composition provided herein comprises or consists of N165, K166, K169, and D234. In some embodiments, the epitope for the anti-CD30L in the pharmaceutical composition provided herein comprises or consists of N165, K166, K169, and H167. In some embodiments, the epitope for the anti-CD30L in the pharmaceutical composition provided herein comprises or consists of N165, K166, K169, and S217. In some embodiments, the epitope for the anti-CD30L in the pharmaceutical composition provided herein comprises or consists of N165, K166, D234, and H167. In some embodiments, the epitope for the anti-CD30L in the pharmaceutical composition provided herein comprises or consists of N165, K166, D234, and S217. In some embodiments, the epitope for the anti-CD30L in the pharmaceutical composition provided herein comprises or consists of N165, K166, H167, and S217. In certain embodiments, the epitope for the anti-CD30L in the pharmaceutical composition provided herein comprises or consists of N165, I168, K169, and D234. In certain embodiments, the epitope for the anti-CD30L in the pharmaceutical composition provided herein comprises or consists of N165, I168, K169, and H167. In some embodiments, the epitope for the anti-CD30L in the pharmaceutical composition provided herein comprises or consists of N165, I168, K169, and S217. In certain embodiments, the epitope for the anti-CD30L in the pharmaceutical composition provided herein comprises or consists of N165, I168, D234, and H167. In some embodiments, the epitope for the anti-CD30L in the pharmaceutical composition provided herein comprises or consists of N165, I168, D234, and S217. In other embodiments, the epitope for the anti-CD30L in the pharmaceutical composition provided herein comprises or consists of N165, I168, H167, and S217. In yet other embodiments, the epitope for the anti-CD30L in the pharmaceutical composition provided herein comprises or consists of N165, K169, D234, and H167. In some embodiments, the epitope for the anti-CD30L in the pharmaceutical composition provided herein comprises or consists of N165, K169, D234, and S217. In certain embodiments, the epitope for the anti-CD30L in the pharmaceutical composition provided herein comprises or consists of N165, K169, H167, and S217. In other embodiments, the epitope for the anti-CD30L in the pharmaceutical composition provided herein comprises or consists of N165, D234, H167, and S217. In further embodiments, the epitope for the anti-CD30L in the pharmaceutical composition provided herein comprises or consists of K166, I168, K169, and D234. In some embodiments, the epitope for the anti-CD30L in the pharmaceutical composition provided herein comprises or consists of K166, I168, K169, and H167. In certain embodiments, the epitope for the anti-CD30L in the pharmaceutical composition provided herein comprises or consists of K166, I168, K169, and S217. In further embodiments, the epitope for the anti-CD30L in the pharmaceutical composition provided herein comprises or consists of K166, I168, D234, and H167. In other embodiments, the epitope for the anti-CD30L in the pharmaceutical composition provided herein comprises or consists of K166, I168, D234, and S217. In some embodiments, the epitope for the anti-CD30L in the pharmaceutical composition provided herein comprises or consists of K166, I168, H167, and S217. In some embodiments, the epitope for the anti-CD30L in the pharmaceutical composition provided herein comprises or consists of K166, K169, D234, and H167. In some embodiments, the epitope for the anti-CD30L in the pharmaceutical composition provided herein comprises or consists of K166, K169, D234, and S217. In some embodiments, the epitope for the anti-CD30L in the pharmaceutical composition provided herein comprises or consists of K166, K169, H167, and S217. In some embodiments, the epitope for the anti-CD30L in the pharmaceutical composition provided herein comprises or consists of K166, D234, H167, and S217. In some embodiments, the epitope for the anti-CD30L in the pharmaceutical composition provided herein comprises or consists of I168, K169, D234, and H167. In some embodiments, the epitope for the anti-CD30L in the pharmaceutical composition provided herein comprises or consists of I168, K169, D234, and S217. In some embodiments, the epitope for the anti-CD30L in the pharmaceutical composition provided herein comprises or consists of I168, K169, H167, and S217. In some embodiments, the epitope for the anti-CD30L in the pharmaceutical composition provided herein comprises or consists of I168, D234, H167, and S217. In some embodiments, the epitope for the anti-CD30L in the pharmaceutical composition provided herein comprises or consists of K169, D234, H167, and S217. In other embodiments, the epitope for the anti-CD30L in the pharmaceutical composition provided herein comprises or consists of N165, K166, I168, and D118. In yet other embodiments, the epitope for the anti-CD30L in the pharmaceutical composition provided herein comprises or consists of N165, K166, K169, and D118. In further embodiments, the epitope for the anti-CD30L in the pharmaceutical composition provided herein comprises or consists of N165, K166, D234, and D118. In some embodiments, the epitope for the anti-CD30L in the pharmaceutical composition provided herein comprises or consists of N165, K166, H167, and D118. In certain embodiments, the epitope for the anti-CD30L in the pharmaceutical composition provided herein comprises or consists of N165, K166, S217, and D118. In some embodiments, the epitope for the anti-CD30L in the pharmaceutical composition provided herein comprises or consists of N165, I168, K169, and D118. In certain embodiments, the epitope for the anti-CD30L in the pharmaceutical composition provided herein comprises or consists of N165, I168, D234, and D118. In some embodiments, the epitope for the anti-CD30L in the pharmaceutical composition provided herein comprises or consists of N165, I168, H167, and D118. In other embodiments, the epitope for the anti-CD30L in the pharmaceutical composition provided herein comprises or consists of N165, I168, S217, and D118. In yet other embodiments, the epitope for the anti-CD30L in the pharmaceutical composition provided herein comprises or consists of N165, K169, D234, and D118. In some embodiments, the epitope for the anti-CD30L in the pharmaceutical composition provided herein comprises or consists of N165, K169, H167, and D118. In other embodiments, the epitope for the anti-CD30L in the pharmaceutical composition provided herein comprises or consists of N165, K169, S217, and D118. In some embodiments, the epitope for the anti-CD30L in the pharmaceutical composition provided herein comprises or consists of N165, D234, H167, and D118. In certain embodiments, the epitope for the anti-CD30L in the pharmaceutical composition provided herein comprises or consists of N165, D234, S217, and D118. In other embodiments, the epitope for the anti-CD30L in the pharmaceutical composition provided herein comprises or consists of N165, H167, S217, and D118. In some embodiments, the epitope for the anti-CD30L in the pharmaceutical composition provided herein comprises or consists of K166, I168, K169, and D118. In certain embodiments, the epitope for the anti-CD30L in the pharmaceutical composition provided herein comprises or consists of K166, I168, D234, and D118. In some embodiments, the epitope for the anti-CD30L in the pharmaceutical composition provided herein comprises or consists of K166, I168, H167, and D118. In certain embodiments, the epitope for the anti-CD30L in the pharmaceutical composition provided herein comprises or consists of K166, I168, S217, and D118. In other embodiments, the epitope for the anti-CD30L in the pharmaceutical composition provided herein comprises or consists of K166, K169, D234, and D118. In yet other embodiments, the epitope for the anti-CD30L in the pharmaceutical composition provided herein comprises or consists of K166, K169, H167, and D118. In some embodiments, the epitope for the anti-CD30L in the pharmaceutical composition provided herein comprises or consists of K166, K169, S217, and D118. In some embodiments, the epitope for the anti-CD30L in the pharmaceutical composition provided herein comprises or consists of K166, D234, H167, and D118. In some embodiments, the epitope for the anti-CD30L in the pharmaceutical composition provided herein comprises or consists of K166, D234, S217, and D118. In some embodiments, the epitope for the anti-CD30L in the pharmaceutical composition provided herein comprises or consists of K166, H167, S217, and D118. In yet other embodiments, the epitope for the anti-CD30L in the pharmaceutical composition provided herein comprises or consists of I168, K169, D234, and D118. In other embodiments, the epitope for the anti-CD30L in the pharmaceutical composition provided herein comprises or consists of I168, K169, H167, and D118. In some embodiments, the epitope for the anti-CD30L in the pharmaceutical composition provided herein comprises or consists of I168, K169, S217, and D118. In certain embodiments, the epitope for the anti-CD30L in the pharmaceutical composition provided herein comprises or consists of I168, D234, H167, and D118. In other embodiments, the epitope for the anti-CD30L in the pharmaceutical composition provided herein comprises or consists of I168, D234, S217, and D118. In yet other embodiments, the epitope for the anti-CD30L in the pharmaceutical composition provided herein comprises or consists of I168, H167, S217, and D118. In some embodiments, the epitope for the anti-CD30L in the pharmaceutical composition provided herein comprises or consists of K169, D234, H167, and D118. In some embodiments, the epitope for the anti-CD30L in the pharmaceutical composition provided herein comprises or consists of K169, D234, S217, and D118. In some embodiments, the epitope for the anti-CD30L in the pharmaceutical composition provided herein comprises or consists of K169, H167, S217, and D118. In some embodiments, the epitope for the anti-CD30L in the pharmaceutical composition provided herein comprises or consists of D234, H167, S217, and D118. In certain embodiments, the epitope for the anti-CD30L in the pharmaceutical composition provided herein comprises or consists of N165, K166, I168, K169, and D234. In some embodiments, the epitope for the anti-CD30L in the pharmaceutical composition provided herein comprises or consists of N165, K166, I168, K169, and H167. In certain embodiments, the epitope for the anti-CD30L in the pharmaceutical composition provided herein comprises or consists of N165, K166, I168, K169, and S217. In some embodiments, the epitope for the anti-CD30L in the pharmaceutical composition provided herein comprises or consists of N165, K166, I168, D234, and H167. In certain embodiments, the epitope for the anti-CD30L in the pharmaceutical composition provided herein comprises or consists of N165, K166, I168, D234, and S217. In some embodiments, the epitope for the anti-CD30L in the pharmaceutical composition provided herein comprises or consists of N165, K166, I168, H167, and S217. In certain embodiments, the epitope for the anti-CD30L in the pharmaceutical composition provided herein comprises or consists of N165, K166, K169, D234, and H167. In some embodiments, the epitope for the anti-CD30L in the pharmaceutical composition provided herein comprises or consists of N165, K166, K169, D234, and S217. In certain embodiments, the epitope for the anti-CD30L in the pharmaceutical composition provided herein comprises or consists of N165, K166, K169, H167, and S217. In some embodiments, the epitope for the anti-CD30L in the pharmaceutical composition provided herein comprises or consists of N165, K166, D234, H167, and S217. In certain embodiments, the epitope for the anti-CD30L in the pharmaceutical composition provided herein comprises or consists of N165, I168, K169, D234, and H167. In some embodiments, the epitope for the anti-CD30L in the pharmaceutical composition provided herein comprises or consists of N165, I168, K169, D234, and S217. In certain embodiments, the epitope for the anti-CD30L in the pharmaceutical composition provided herein comprises or consists of N165, I168, K169, H167, and S217. In some embodiments, the epitope for the anti-CD30L in the pharmaceutical composition provided herein comprises or consists of N165, I168, D234, H167, and S217. In certain embodiments, the epitope for the anti-CD30L in the pharmaceutical composition provided herein comprises or consists of N165, K169, D234, H167, and S217. In some embodiments, the epitope for the anti-CD30L in the pharmaceutical composition provided herein comprises or consists of K166, I168, K169, D234, and H167. In certain embodiments, the epitope for the anti-CD30L in the pharmaceutical composition provided herein comprises or consists of K166, I168, K169, D234, and S217. In some embodiments, the epitope for the anti-CD30L in the pharmaceutical composition provided herein comprises or consists of K166, I168, K169, H167, and S217. In some embodiments, the epitope for the anti-CD30L in the pharmaceutical composition provided herein comprises or consists of K166, I168, D234, H167, and S217. In certain embodiments, the epitope for the anti-CD30L in the pharmaceutical composition provided herein comprises or consists of K166, K169, D234, H167, and S217. In some embodiments, the epitope for the anti-CD30L in the pharmaceutical composition provided herein comprises or consists of I168, K169, D234, H167, and S217. In other embodiments, the epitope for the anti-CD30L in the pharmaceutical composition provided herein comprises or consists of N165, K166, I168, K169, and D118. In further embodiments, the epitope for the anti-CD30L in the pharmaceutical composition provided herein comprises or consists of N165, K166, I168, D234, and D118. In some embodiments, the epitope for the anti-CD30L in the pharmaceutical composition provided herein comprises or consists of N165, K166, I168, H167, and D118. In other embodiments, the epitope for the anti-CD30L in the pharmaceutical composition provided herein comprises or consists of N165, K166, I168, S217, and D118. In some embodiments, the epitope for the anti-CD30L in the pharmaceutical composition provided herein comprises or consists of N165, K166, K169, D234, and D118. In some embodiments, the epitope for the anti-CD30L in the pharmaceutical composition provided herein comprises or consists of N165, K166, K169, H167, and D118. In some embodiments, the epitope for the anti-CD30L in the pharmaceutical composition provided herein comprises or consists of N165, K166, K169, S217, and D118. In some embodiments, the epitope for the anti-CD30L in the pharmaceutical composition provided herein comprises or consists of N165, K166, D234, H167, and D118. In some embodiments, the epitope for the anti-CD30L in the pharmaceutical composition provided herein comprises or consists of N165, K166, D234, S217, and D118. In some embodiments, the epitope for the anti-CD30L in the pharmaceutical composition provided herein comprises or consists of N165, K166, H167, S217, and D118. In certain embodiments, the epitope for the anti-CD30L in the pharmaceutical composition provided herein comprises or consists of N165, I168, K169, D234, and D118. In certain embodiments, the epitope for the anti-CD30L in the pharmaceutical composition provided herein comprises or consists of N165, I168, K169, H167, and D118. In some embodiments, the epitope for the anti-CD30L in the pharmaceutical composition provided herein comprises or consists of N165, I168, K169, S217, and D118. In certain embodiments, the epitope for the anti-CD30L in the pharmaceutical composition provided herein comprises or consists of N165, I168, D234, H167, and D118. In some embodiments, the epitope for the anti-CD30L in the pharmaceutical composition provided herein comprises or consists of N165, I168, D234, S217, and D118. In other embodiments, the epitope for the anti-CD30L in the pharmaceutical composition provided herein comprises or consists of N165, I168, H167, S217, and D118. In yet other embodiments, the epitope for the anti-CD30L in the pharmaceutical composition provided herein comprises or consists of N165, K169, D234, H167, and D118. In some embodiments, the epitope for the anti-CD30L in the pharmaceutical composition provided herein comprises or consists of N165, K169, D234, S217, and D118. In certain embodiments, the epitope for the anti-CD30L in the pharmaceutical composition provided herein comprises or consists of N165, K169, H167, S217, and D118. In other embodiments, the epitope for the anti-CD30L in the pharmaceutical composition provided herein comprises or consists of N165, D234, H167, S217, and D118. In further embodiments, the epitope for the anti-CD30L in the pharmaceutical composition provided herein comprises or consists of K166, I168, K169, D234, and D118. In some embodiments, the epitope for the anti-CD30L in the pharmaceutical composition provided herein comprises or consists of K166, I168, K169, H167, and D118. In certain embodiments, the epitope for the anti-CD30L in the pharmaceutical composition provided herein comprises or consists of K166, I168, K169, S217, and D118. In further embodiments, the epitope for the anti-CD30L in the pharmaceutical composition provided herein comprises or consists of K166, I168, D234, H167, and D118. In other embodiments, the epitope for the anti-CD30L in the pharmaceutical composition provided herein comprises or consists of K166, I168, D234, S217, and D118. In some embodiments, the epitope for the anti-CD30L in the pharmaceutical composition provided herein comprises or consists of K166, I168, H167, S217, and D118. In some embodiments, the epitope for the anti-CD30L in the pharmaceutical composition provided herein comprises or consists of K166, K169, D234, H167, and D118. In some embodiments, the epitope for the anti-CD30L in the pharmaceutical composition provided herein comprises or consists of K166, K169, D234, S217, and D118. In some embodiments, the epitope for the anti-CD30L in the pharmaceutical composition provided herein comprises or consists of K166, K169, H167, S217, and D118. In some embodiments, the epitope for the anti-CD30L in the pharmaceutical composition provided herein comprises or consists of K166, D234, H167, S217, and D118. In some embodiments, the epitope for the anti-CD30L in the pharmaceutical composition provided herein comprises or consists of I168, K169, D234, H167, and D118. In some embodiments, the epitope for the anti-CD30L in the pharmaceutical composition provided herein comprises or consists of I168, K169, D234, S217, and D118. In some embodiments, the epitope for the anti-CD30L in the pharmaceutical composition provided herein comprises or consists of I168, K169, H167, S217, and D118. In some embodiments, the epitope for the anti-CD30L in the pharmaceutical composition provided herein comprises or consists of I168, D234, H167, S217, and D118. In some embodiments, the epitope for the anti-CD30L in the pharmaceutical composition provided herein comprises or consists of K169, D234, H167, S217, and D118. In some embodiments, the epitope for the anti-CD30L in the pharmaceutical composition provided herein comprises or consists of N165, K166, I168, K169, D234, and H167. In certain embodiments, the epitope for the anti-CD30L in the pharmaceutical composition provided herein comprises or consists of N165, K166, I168, K169, D234, and S217. In some embodiments, the epitope for the anti-CD30L in the pharmaceutical composition provided herein comprises or consists of N165, K166, I168, K169, H167, and S217. In certain embodiments, the epitope for the anti-CD30L in the pharmaceutical composition provided herein comprises or consists of N165, K166, I168, D234, H167, and S217. In some embodiments, the epitope for the anti-CD30L in the pharmaceutical composition provided herein comprises or consists of N165, K166, K169, D234, H167, and S217. In certain embodiments, the epitope for the anti-CD30L in the pharmaceutical composition provided herein comprises or consists of N165, I168, K169, D234, H167, and S217. In some embodiments, the epitope for the anti-CD30L in the pharmaceutical composition provided herein comprises or consists of K166, I168, K169, D234, H167, and S217. In certain embodiments, the epitope for the anti-CD30L in the pharmaceutical composition provided herein comprises or consists of N165, K166, I168, K169, D234, and D118. In some embodiments, the epitope for the anti-CD30L in the pharmaceutical composition provided herein comprises or consists of N165, K166, I168, K169, H167, and D118. In certain embodiments, the epitope for the anti-CD30L in the pharmaceutical composition provided herein comprises or consists of N165, K166, I168, K169, S217, and D118. In some embodiments, the epitope for the anti-CD30L in the pharmaceutical composition provided herein comprises or consists of N165, K166, I168, D234, H167, and D118. In certain embodiments, the epitope for the anti-CD30L in the pharmaceutical composition provided herein comprises or consists of N165, K166, I168, D234, S217, and D118. In some embodiments, the epitope for the anti-CD30L in the pharmaceutical composition provided herein comprises or consists of N165, K166, I168, H167, S217, and D118. In certain embodiments, the epitope for the anti-CD30L in the pharmaceutical composition provided herein comprises or consists of N165, K166, K169, D234, H167, and D118. In some embodiments, the epitope for the anti-CD30L in the pharmaceutical composition provided herein comprises or consists of N165, K166, K169, D234, S217, and D118. In certain embodiments, the epitope for the anti-CD30L in the pharmaceutical composition provided herein comprises or consists of N165, K166, K169, H167, S217, and D118. In some embodiments, the epitope for the anti-CD30L in the pharmaceutical composition provided herein comprises or consists of N165, K166, D234, H167, S217, and D118. In certain embodiments, the epitope for the anti-CD30L in the pharmaceutical composition provided herein comprises or consists of N165, I168, K169, D234, H167, and D118. In some embodiments, the epitope for the anti-CD30L in the pharmaceutical composition provided herein comprises or consists of N165, I168, K169, D234, S217, and D118. In certain embodiments, the epitope for the anti-CD30L in the pharmaceutical composition provided herein comprises or consists of N165, I168, K169, H167, S217, and D118. In some embodiments, the epitope for the anti-CD30L in the pharmaceutical composition provided herein comprises or consists of N165, I168, D234, H167, S217, and D118. In certain embodiments, the epitope for the anti-CD30L in the pharmaceutical composition provided herein comprises or consists of N165, K169, D234, H167, S217, and D118. In some embodiments, the epitope for the anti-CD30L in the pharmaceutical composition provided herein comprises or consists of K166, I168, K169, D234, H167, and D118. In certain embodiments, the epitope for the anti-CD30L in the pharmaceutical composition provided herein comprises or consists of K166, I168, K169, D234, S217, and D118. In some embodiments, the epitope for the anti-CD30L in the pharmaceutical composition provided herein comprises or consists of K166, I168, K169, H167, S217, and D118. In some embodiments, the epitope for the anti-CD30L in the pharmaceutical composition provided herein comprises or consists of K166, I168, D234, H167, S217, and D118. In certain embodiments, the epitope for the anti-CD30L in the pharmaceutical composition provided herein comprises or consists of K166, K169, D234, H167, S217, and D118. In some embodiments, the epitope for the anti-CD30L in the pharmaceutical composition provided herein comprises or consists of I168, K169, D234, H167, S217, and D118. In some embodiments, the epitope for the anti-CD30L in the pharmaceutical composition provided herein comprises or consists of any one of N165, K166, I168, K169, D234, H167, and S217. In certain embodiments, the epitope for the anti-CD30L in the pharmaceutical composition provided herein comprises or consists of any one of N165, K166, I168, K169, D234, H167, and D118. In some embodiments, the epitope for the anti-CD30L in the pharmaceutical composition provided herein comprises or consists of any one of N165, K166, I168, K169, D234, S217, and D118. In some embodiments, the epitope for the anti-CD30L in the pharmaceutical composition provided herein comprises or consists of any one of N165, K166, I168, K169, H167, S217, and D118. In some embodiments, the epitope for the anti-CD30L in the pharmaceutical composition provided herein comprises or consists of any one of N165, K166, I168, D234, H167, S217, and D118. In some embodiments, the epitope for the anti-CD30L in the pharmaceutical composition provided herein comprises or consists of any one of N165, K166, K169, D234, H167, S217, and D118. In some embodiments, the epitope for the anti-CD30L in the pharmaceutical composition provided herein comprises or consists of any one of N165, I168, K169, D234, H167, S217, and D118. In some embodiments, the epitope for the anti-CD30L in the pharmaceutical composition provided herein comprises or consists of any one of K166, I168, K169, D234, H167, S217, and D118. In some embodiments, the epitope for the anti-CD30L in the pharmaceutical composition provided herein comprises or consists of any two of N165, K166, I168, K169, D234, H167, S217, and D118 in any combination or permutation. In some embodiments, the epitope for the anti-CD30L in the pharmaceutical composition provided herein comprises or consists of any three of N165, K166, I168, K169, D234, H167, S217, and D118 in any combination or permutation. In some embodiments, the epitope for the anti-CD30L in the pharmaceutical composition provided herein comprises or consists of any four of N165, K166, I168, K169, D234, H167, S217, and D118, in any combination or permutation. In some embodiments, the epitope for the anti-CD30L in the pharmaceutical composition provided herein comprises or consists of any five of N165, K166, I168, K169, D234, H167, S217, and D118, in any combination or permutation. In some embodiments, the epitope for the anti-CD30L in the pharmaceutical composition provided herein comprises or consists of any six of N165, K166, I168, K169, D234, H167, S217, and D118, in any combination or permutation. In some embodiments, the epitope for the anti-CD30L in the pharmaceutical composition provided herein comprises or consists of seven of N165, K166, I168, K169, D234, H167, S217, and D118, in any combination or permutation. In some embodiments, the epitope for the anti-CD30L in the pharmaceutical composition provided herein comprises or consists of eight of N165, K166, I168, K169, D234, H167, S217, and D118, in any permutation. The position of the amino acids in this paragraph are numbered according to the amino acid sequence of CD30L as set forth in SEQ ID NO:33.

In some embodiments of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in any one of SEQ ID NOs: 110-119, 629, 636, and 712-723; (b) a CDR-H2 comprising the amino acid sequence set forth in any one of SEQ ID NOs: 150-159, 643, 650, and 724-735; (c) a CDR-H3 comprising the amino acid sequence set forth in any one of SEQ ID NOs: 190-199, 657, 664, and 736-743; (d) a CDR-L1 comprising the amino acid sequence set forth in any one of SEQ ID NOs: 310-319, 671, 678, and 744-751; (e) a CDR-L2 comprising the amino acid sequence set forth in any one of SEQ ID NOs: 350-359, 685, 692, and 752-759; and/or (f) a CDR-L3 comprising the amino acid sequence set forth in any one of SEQ ID NOs: 390-399, 699, 706, and 760-765.

In certain embodiments of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in any one of SEQ ID NOs: 110-114, 629, 636, and 712-723; (b) a CDR-H2 comprising the amino acid sequence set forth in any one of SEQ ID NOs: 150-154, 643, 650, and 724-735; (c) a CDR-H3 comprising the amino acid sequence set forth in any one of SEQ ID NOs: 190-194, 657, 664, and 736-743; (d) a CDR-L1 comprising the amino acid sequence set forth in any one of SEQ ID NOs: 310-314, 671, 678, and 744-751; (e) a CDR-L2 comprising the amino acid sequence set forth in any one of SEQ ID NOs: 350-354, 685, 692, and 752-759; and/or (f) a CDR-L3 comprising the amino acid sequence set forth in any one of SEQ ID NOs: 390-394, 699, 706, and 760-765.

In other embodiments of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in any one of SEQ ID NOs: 115-119, 629, 636, and 712-723; (b) a CDR-H2 comprising the amino acid sequence set forth in any one of SEQ ID NOs: 155-159, 643, 650, and 724-735; (c) a CDR-H3 comprising the amino acid sequence set forth in any one of SEQ ID NOs: 195-199, 657, 664, and 736-743; (d) a CDR-L1 comprising the amino acid sequence set forth in any one of SEQ ID NOs: 315-319, 671, 678, and 744-751; (e) a CDR-L2 comprising the amino acid sequence set forth in any one of SEQ ID NOs: 355-359, 685, 692, and 752-759; and/or (f) a CDR-L3 comprising the amino acid sequence set forth in any one of SEQ ID NOs: 395-399, 699, 706, and 760-765.

In some embodiments of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in any one of SEQ ID NOs: 120-129, and 712-723; (b) a CDR-H2 comprising the amino acid sequence set forth in any one of SEQ ID NOs: 160-169, and 724-735; (c) a CDR-H3 comprising the amino acid sequence set forth in any one of SEQ ID NOs: 200-209, and 736-743; (d) a CDR-L1 comprising the amino acid sequence set forth in any one of SEQ ID NOs: 320-329, and 744-751; (e) a CDR-L2 comprising the amino acid sequence set forth in any one of SEQ ID NOs: 360-369, and 752-759; and/or (f) a CDR-L3 comprising the amino acid sequence set forth in any one of SEQ ID NOs: 400-409, and 760-765.

In certain embodiments of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in any one of SEQ ID NOs: 120-124, and 712-723; (b) a CDR-H2 comprising the amino acid sequence set forth in any one of SEQ ID NOs: 160-164, and 724-735; (c) a CDR-H3 comprising the amino acid sequence set forth in any one of SEQ ID NOs: 200-204, and 736-743; (d) a CDR-L1 comprising the amino acid sequence set forth in any one of SEQ ID NOs: 320-324, and 744-751; (e) a CDR-L2 comprising the amino acid sequence set forth in any one of SEQ ID NOs: 360-364, and 752-759; and/or (f) a CDR-L3 comprising the amino acid sequence set forth in any one of SEQ ID NOs: 400-404, and 760-765.

In other embodiments of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in any one of SEQ ID NOs: 125-129, and 712-723; (b) a CDR-H2 comprising the amino acid sequence set forth in any one of SEQ ID NOs: 165-169, and 724-735; (c) a CDR-H3 comprising the amino acid sequence set forth in any one of SEQ ID NOs: 205-209, and 736-743; (d) a CDR-L1 comprising the amino acid sequence set forth in any one of SEQ ID NOs: 325-329, and 744-751; (e) a CDR-L2 comprising the amino acid sequence set forth in any one of SEQ ID NOs: 365-369, and 752-759; and/or (f) a CDR-L3 comprising the amino acid sequence set forth in any one of SEQ ID NOs: 405-409, and 760-765.

In some embodiments of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in any one of SEQ ID NOs: 130-139, and 712-723; (b) a CDR-H2 comprising the amino acid sequence set forth in any one of SEQ ID NOs: 170-179, and 724-735; (c) a CDR-H3 comprising the amino acid sequence set forth in any one of SEQ ID NOs: 210-219, and 736-743; (d) a CDR-L1 comprising the amino acid sequence set forth in any one of SEQ ID NOs: 330-339, and 744-751; (e) a CDR-L2 comprising the amino acid sequence set forth in any one of SEQ ID NOs: 370-379, and 752-759; and/or (f) a CDR-L3 comprising the amino acid sequence set forth in any one of SEQ ID NOs: 410-419, and 760-765.

In certain embodiments of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in any one of SEQ ID NOs: 130-134, and 712-723; (b) a CDR-H2 comprising the amino acid sequence set forth in any one of SEQ ID NOs: 170-174, and 724-735; (c) a CDR-H3 comprising the amino acid sequence set forth in any one of SEQ ID NOs: 210-214, and 736-743; (d) a CDR-L1 comprising the amino acid sequence set forth in any one of SEQ ID NOs: 330-334, and 744-751; (e) a CDR-L2 comprising the amino acid sequence set forth in any one of SEQ ID NOs: 370-374, and 752-759; and/or (f) a CDR-L3 comprising the amino acid sequence set forth in any one of SEQ ID NOs: 410-414, and 760-765.

In other embodiments of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in any one of SEQ ID NOs: 135-139, and 712-723; (b) a CDR-H2 comprising the amino acid sequence set forth in any one of SEQ ID NOs: 175-179, and 724-735; (c) a CDR-H3 comprising the amino acid sequence set forth in any one of SEQ ID NOs: 215-219, and 736-743; (d) a CDR-L1 comprising the amino acid sequence set forth in any one of SEQ ID NOs: 335-339, and 744-751; (e) a CDR-L2 comprising the amino acid sequence set forth in any one of SEQ ID NOs: 375-379, and 752-759; and/or (f) a CDR-L3 comprising the amino acid sequence set forth in any one of SEQ ID NOs: 415-419, and 760-765.

In some embodiments of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in any one of SEQ ID NOs: 220-224, and 712-723; (b) a CDR-H2 comprising the amino acid sequence set forth in any one of SEQ ID NOs: 235-239, and 724-735; (c) a CDR-H3 comprising the amino acid sequence set forth in any one of SEQ ID NOs: 250-254, and 736-743; (d) a CDR-L1 comprising the amino acid sequence set forth in any one of SEQ ID NOs: 420-424, and 744-751; (e) a CDR-L2 comprising the amino acid sequence set forth in any one of SEQ ID NOs: 435-439, and 752-759; and/or (f) a CDR-L3 comprising the amino acid sequence set forth in any one of SEQ ID NOs: 450-454, and 760-765.

In certain embodiments of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in any one of SEQ ID NOs: 225-229, and 712-723; (b) a CDR-H2 comprising the amino acid sequence set forth in any one of SEQ ID NOs: 240-244, and 724-735; (c) a CDR-H3 comprising the amino acid sequence set forth in any one of SEQ ID NOs: 255-259, and 736-743; (d) a CDR-L1 comprising the amino acid sequence set forth in any one of SEQ ID NOs: 425-429, and 744-751; (e) a CDR-L2 comprising the amino acid sequence set forth in any one of SEQ ID NOs: 440-444, and 752-759; and/or (f) a CDR-L3 comprising the amino acid sequence set forth in any one of SEQ ID NOs: 455-459, and 760-765.

In other embodiments of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in any one of SEQ ID NOs: 230-234, and 712-723; (b) a CDR-H2 comprising the amino acid sequence set forth in any one of SEQ ID NOs: 245-249, and 724-735; (c) a CDR-H3 comprising the amino acid sequence set forth in any one of SEQ ID NOs: 260-264, and 736-743; (d) a CDR-L1 comprising the amino acid sequence set forth in any one of SEQ ID NOs: 430-434, and 744-751; (e) a CDR-L2 comprising the amino acid sequence set forth in any one of SEQ ID NOs: 445-449, and 752-759; and/or (f) a CDR-L3 comprising the amino acid sequence set forth in any one of SEQ ID NOs: 460-464, and 760-765.

In one embodiment of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in any one of SEQ ID NOs: 465-469, 631, 638, and 712-723; (b) a CDR-H2 comprising the amino acid sequence set forth in any one of SEQ ID NOs: 490-494, 645, 652, and 724-735; (c) a CDR-H3 comprising the amino acid sequence set forth in any one of SEQ ID NOs: 528-532, 659, 666, and 736-743; (d) a CDR-L1 comprising the amino acid sequence set forth in any one of SEQ ID NOs: 553-557, 673, 680, and 744-751; (e) a CDR-L2 comprising the amino acid sequence set forth in any one of SEQ ID NOs: 578-582, 687, 694, and 752-759; and/or (f) a CDR-L3 comprising the amino acid sequence set forth in any one of SEQ ID NOs: 603-607, 701, 708, and 760-765;

In another embodiment of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in any one of SEQ ID NOs: 470-474, 632, 639, and 712-723; (b) a CDR-H2 comprising the amino acid sequence set forth in any one of SEQ ID NOs: 495-499, 646, 653, and 724-735; (c) a CDR-H3 comprising the amino acid sequence set forth in any one of SEQ ID NOs: 533-537, 660, 667, and 736-743; (d) a CDR-L1 comprising the amino acid sequence set forth in any one of SEQ ID NOs: 558-562, 674, 681, and 744-751; (e) a CDR-L2 comprising the amino acid sequence set forth in any one of SEQ ID NOs: 583-587, 688, 695, and 752-759; and/or (f) a CDR-L3 comprising the amino acid sequence set forth in any one of SEQ ID NOs: 608-612, 702, 709, and 760-765;

In a further embodiment of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in any one of SEQ ID NOs: 475-479, 633, 640, and 712-723; (b) a CDR-H2 comprising the amino acid sequence set forth in any one of SEQ ID NOs: 513-517, 647, 654, and 724-735; (c) a CDR-H3 comprising the amino acid sequence set forth in any one of SEQ ID NOs: 538-542, 661, 668, and 736-743; (d) a CDR-L1 comprising the amino acid sequence set forth in any one of SEQ ID NOs: 563-567, 675, 682, and 744-751; (e) a CDR-L2 comprising the amino acid sequence set forth in any one of SEQ ID NOs: 588-592, 689, 696, and 752-759; and/or (f) a CDR-L3 comprising the amino acid sequence set forth in any one of SEQ ID NOs: 613-617, 703, 710, and 760-765;

In yet another embodiment of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in any one of SEQ ID NOs: 480-484, 630, 637, and 712-723; (b) a CDR-H2 comprising the amino acid sequence set forth in any one of SEQ ID NOs: 518-522, 644, 651, and 724-735; (c) a CDR-H3 comprising the amino acid sequence set forth in any one of SEQ ID NOs: 543-547, 658, 665, and 736-743; (d) a CDR-L1 comprising the amino acid sequence set forth in any one of SEQ ID NOs: 568-572, 672, 679, and 744-751; (e) a CDR-L2 comprising the amino acid sequence set forth in any one of SEQ ID NOs: 593-597, 686, 693, and 752-759; and/or (f) a CDR-L3 comprising the amino acid sequence set forth in any one of SEQ ID NOs: 618-622, 700, 707, and 760-765; or

In one embodiment of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in any one of SEQ ID Nos: 485-489, 634, 641, and 712-723; (b) a CDR-H2 comprising the amino acid sequence set forth in any one of SEQ ID Nos: 523-527, 648, 655, and 724-735; (c) a CDR-H3 comprising the amino acid sequence set forth in any one of SEQ ID Nos: 548-552, 662, 669, and 736-743; (d) a CDR-L1 comprising the amino acid sequence set forth in any one of SEQ ID Nos: 573-577, 676, 683, and 744-751; € a CDR-L2 comprising the amino acid sequence set forth in any one of SEQ ID Nos: 598-602, 690, 697, and 752-759; and/or (f) a CDR-L3 comprising the amino acid sequence set forth in any one of SEQ ID Nos: 623-627, 704, 711, and 760-765.

The disclosure further provides that the sequence and structural features in the CDR sequences common to the antibodies that confer the anti-CD30L antibody in the pharmaceutical composition the binding affinity, specificity, and the capacity to functionally block both the CD30-CD30L interaction and the CD30L-mediated CD30 signaling and IL-8 releasing. Accordingly, also provided herein are pharmaceutical compositions comprising anti-CD30L antibodies with consensus CDR sequences that confer the anti-CD30L antibody the binding affinity, specificity, and the capacity to functionally block both the CD30-CD30L interaction and the CD30L-mediated CD30 signaling and IL-8 releasing. In one embodiment of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in any one of SEQ ID NOs: 712-723; (b) a CDR-H2 comprising the amino acid sequence set forth in any one of SEQ ID NOs: 724-735; (c) a CDR-H3 comprising the amino acid sequence set forth in any one of SEQ ID NOs: 736-743; (d) a CDR-L1 comprising the amino acid sequence set forth in any one of SEQ ID NOs: 744-751; (e) a CDR-L2 comprising the amino acid sequence set forth in any one of SEQ ID NOs: 752-759; and/or (f) a CDR-L3 comprising the amino acid sequence set forth in any one of SEQ ID NOs: 760-765. In another embodiment of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in any one of SEQ ID NOs: 712, 714, 716, 718, 720, and 722; (b) a CDR-H2 comprising the amino acid sequence set forth in any one of SEQ ID NOs: 724, 726, 728, 730, 732, and 734; (c) a CDR-H3 comprising the amino acid sequence set forth in any one of SEQ ID NOs: 736, 738, 740, and 742; (d) a CDR-L1 comprising the amino acid sequence set forth in any one of SEQ ID NOs: 744, 746, 748, and 750; (e) a CDR-L2 comprising the amino acid sequence set forth in any one of SEQ ID NOs: 752, 754, 756, and 758; and/or (f) a CDR-L3 comprising the amino acid sequence set forth in any one of SEQ ID NOs: 760, 762, and 764. In a further embodiment of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in any one of SEQ ID NOs: 713, 715, 717, 719, 721, and 723; (b) a CDR-H2 comprising the amino acid sequence set forth in any one of SEQ ID NOs: 725, 727, 729, 731, 733, and 735; (c) a CDR-H3 comprising the amino acid sequence set forth in any one of SEQ ID NOs: 737, 739, 741, and 743; (d) a CDR-L1 comprising the amino acid sequence set forth in any one of SEQ ID NOs: 745, 747, 749, and 751; (e) a CDR-L2 comprising the amino acid sequence set forth in any one of SEQ ID NOs: 753, 755, 757, and 759; and/or (f) a CDR-L3 comprising the amino acid sequence set forth in any one of SEQ ID NOs: 761, 763, and 765. In yet another embodiment of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in SEQ ID NO: 712; (b) a CDR-H2 comprising the amino acid sequence set forth in SEQ ID NO: 730; (c) a CDR-H3 comprising the amino acid sequence set forth in SEQ ID NO: 736; (d) a CDR-L1 comprising the amino acid sequence set forth in SEQ ID NO: 744; (e) a CDR-L2 comprising the amino acid sequence set forth SEQ ID NO: 752; and/or (f) a CDR-L3 comprising the amino acid sequence set forth SEQ ID NO: 760. In one embodiment of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in SEQ ID NO: 713; (b) a CDR-H2 comprising the amino acid sequence set forth in SEQ ID NO: 731; (c) a CDR-H3 comprising the amino acid sequence set forth in SEQ ID NO: 737; (d) a CDR-L1 comprising the amino acid sequence set forth in SEQ ID NO: 745; (e) a CDR-L2 comprising the amino acid sequence set forth SEQ ID NO: 753; and/or (f) a CDR-L3 comprising the amino acid sequence set forth SEQ ID NO: 761. In another embodiment of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in SEQ ID NO: 712; (b) a CDR-H2 comprising the amino acid sequence set forth in SEQ ID NO: 724; (c) a CDR-H3 comprising the amino acid sequence set forth in SEQ ID NO: 736; (d) a CDR-L1 comprising the amino acid sequence set forth in SEQ ID NO: 744; (e) a CDR-L2 comprising the amino acid sequence set forth SEQ ID NO: 752; and/or (f) a CDR-L3 comprising the amino acid sequence set forth SEQ ID NO: 760. In a further embodiment of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in SEQ ID NO: 713; (b) a CDR-H2 comprising the amino acid sequence set forth in SEQ ID NO: 725; (c) a CDR-H3 comprising the amino acid sequence set forth in SEQ ID NO: 737; (d) a CDR-L1 comprising the amino acid sequence set forth in SEQ ID NO: 745; (e) a CDR-L2 comprising the amino acid sequence set forth SEQ ID NO: 753; and/or (f) a CDR-L3 comprising the amino acid sequence set forth SEQ ID NO: 761. In yet another embodiment of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in SEQ ID NO: 714; (b) a CDR-H2 comprising the amino acid sequence set forth in SEQ ID NO: 726; (c) a CDR-H3 comprising the amino acid sequence set forth in SEQ ID NO: 736; (d) a CDR-L1 comprising the amino acid sequence set forth in SEQ ID NO: 744; (e) a CDR-L2 comprising the amino acid sequence set forth SEQ ID NO: 752; and/or (f) a CDR-L3 comprising the amino acid sequence set forth SEQ ID NO: 760. In one embodiment of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in SEQ ID NO: 715; (b) a CDR-H2 comprising the amino acid sequence set forth in SEQ ID NO: 727; (c) a CDR-H3 comprising the amino acid sequence set forth in SEQ ID NO: 737; (d) a CDR-L1 comprising the amino acid sequence set forth in SEQ ID NO: 745; (e) a CDR-L2 comprising the amino acid sequence set forth SEQ ID NO: 753; and/or (f) a CDR-L3 comprising the amino acid sequence set forth SEQ ID NO: 761. In another embodiment of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in SEQ ID NO: 716; (b) a CDR-H2 comprising the amino acid sequence set forth in SEQ ID NO: 728; (c) a CDR-H3 comprising the amino acid sequence set forth in SEQ ID NO: 736; (d) a CDR-L1 comprising the amino acid sequence set forth in SEQ ID NO: 744; (e) a CDR-L2 comprising the amino acid sequence set forth SEQ ID NO: 752; and/or (f) a CDR-L3 comprising the amino acid sequence set forth SEQ ID NO: 760. In yet another embodiment of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in SEQ ID NO: 717; (b) a CDR-H2 comprising the amino acid sequence set forth in SEQ ID NO: 729; (c) a CDR-H3 comprising the amino acid sequence set forth in SEQ ID NO: 737; (d) a CDR-L1 comprising the amino acid sequence set forth in SEQ ID NO: 745; (e) a CDR-L2 comprising the amino acid sequence set forth SEQ ID NO: 753; and/or (f) a CDR-L3 comprising the amino acid sequence set forth SEQ ID NO: 761. In one embodiment of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in SEQ ID NO: 718; (b) a CDR-H2 comprising the amino acid sequence set forth in SEQ ID NO: 730; (c) a CDR-H3 comprising the amino acid sequence set forth in SEQ ID NO: 738; (d) a CDR-L1 comprising the amino acid sequence set forth in SEQ ID NO: 746; (e) a CDR-L2 comprising the amino acid sequence set forth SEQ ID NO: 754; and/or (f) a CDR-L3 comprising the amino acid sequence set forth SEQ ID NO: 762. In one embodiment of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in SEQ ID NO: 719; (b) a CDR-H2 comprising the amino acid sequence set forth in SEQ ID NO: 731; (c) a CDR-H3 comprising the amino acid sequence set forth in SEQ ID NO: 739; (d) a CDR-L1 comprising the amino acid sequence set forth in SEQ ID NO: 747; (e) a CDR-L2 comprising the amino acid sequence set forth SEQ ID NO: 755; and/or (f) a CDR-L3 comprising the amino acid sequence set forth SEQ ID NO: 763. In another embodiment of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in SEQ ID NO: 720; (b) a CDR-H2 comprising the amino acid sequence set forth in SEQ ID NO: 732; (c) a CDR-H3 comprising the amino acid sequence set forth in SEQ ID NO: 740; (d) a CDR-L1 comprising the amino acid sequence set forth in SEQ ID NO: 748; (e) a CDR-L2 comprising the amino acid sequence set forth SEQ ID NO: 756; and/or (f) a CDR-L3 comprising the amino acid sequence set forth SEQ ID NO: 760. In a further embodiment of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in SEQ ID NO: 721; (b) a CDR-H2 comprising the amino acid sequence set forth in SEQ ID NO: 733; (c) a CDR-H3 comprising the amino acid sequence set forth in SEQ ID NO: 741; (d) a CDR-L1 comprising the amino acid sequence set forth in SEQ ID NO: 749; (e) a CDR-L2 comprising the amino acid sequence set forth SEQ ID NO: 757; and/or (f) a CDR-L3 comprising the amino acid sequence set forth SEQ ID NO: 761. In a further embodiment of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in SEQ ID NO: 722; (b) a CDR-H2 comprising the amino acid sequence set forth in SEQ ID NO: 734; (c) a CDR-H3 comprising the amino acid sequence set forth in SEQ ID NO: 742; (d) a CDR-L1 comprising the amino acid sequence set forth in SEQ ID NO: 750; (e) a CDR-L2 comprising the amino acid sequence set forth SEQ ID NO: 758; and/or (f) a CDR-L3 comprising the amino acid sequence set forth SEQ ID NO: 764. In yet another embodiment of the pharmaceutical composition comprising an anti-CD30L antibody or antigen binding fragment provided herein, including in Section 2 and this Section (Section 4.5), the antibody or antigen binding fragment thereof comprises: (a) a CDR-H1 comprising the amino acid sequence set forth in SEQ ID NO: 723; (b) a CDR-H2 comprising the amino acid sequence set forth in SEQ ID NO: 735; (c) a CDR-H3 comprising the amino acid sequence set forth in SEQ ID NO: 743; (d) a CDR-L1 comprising the amino acid sequence set forth in SEQ ID NO: 751; (e) a CDR-L2 comprising the amino acid sequence set forth SEQ ID NO: 759; and/or (f) a CDR-L3 comprising the amino acid sequence set forth SEQ ID NO: 765. In some embodiments of the pharmaceutical composition comprising an anti-CD30L provided herein, including in Section 2 and this Section 4.5 (such as those of this paragraph), the CDR-H1 of the anti-CD30L comprises the amino acid sequence set forth in any one of SEQ ID NOs: 105-109, 628, 635, 115-119, 629, 636, 480-484, 630, 637, 465-469, 631, 638, 470-474, 632, 639, 475-479, 633, 640, 485-489, 634, and 641. In some embodiments of the pharmaceutical composition comprising an anti-CD30L provided herein, including in Section 2 and this Section 4.5 (such as those of this paragraph), the CDR-H2 of the anti-CD30L comprises the amino acid sequence set forth in any one of SEQ ID NOs: 145-149, 642, 649, 155-159, 643, 650, 518-522, 644, 651, 490-494, 645, 652, 495-499, 646, 653, 513-517, 647, 654, 523-527, 648, and 655. In some embodiments of the pharmaceutical composition comprising an anti-CD30L provided herein, including in Section 2 and this Section 4.5 (such as those of this paragraph), the CDR-H3 of the anti-CD30L comprises the amino acid sequence set forth in any one of SEQ ID NOs: 185-189, 656, 663, 195-199, 657, 664, 543-547, 658, 665, 528-532, 659, 666, 533-537, 660, 667, 538-542, 661, 668, 548-552, 662, and 669. In some embodiments of the pharmaceutical composition comprising an anti-CD30L provided herein, including in Section 2 and this Section 4.5 (such as those of this paragraph), the CDR-L1 of the anti-CD30L comprises the amino acid sequence set forth in any one of SEQ ID NOs: 305-309, 670, 677, 315-319, 671, 678, 568-572, 672, 679, 553-557, 673, 680, 558-562, 674, 681, 563-567, 675, 682, 573-577, 676, and 683. In some embodiments of the pharmaceutical composition comprising an anti-CD30L provided herein, including in Section 2 and this Section 4.5 (such as those of this paragraph), the CDR-L2 of the anti-CD30L comprises the amino acid sequence set forth in any one of SEQ ID NOs: 345-349, 684, 691, 355-359, 685, 692, 593-597, 686, 693, 578-582, 687, 694, 583-587, 688, 695, 588-592, 689, 696, 598-602, 690, and 697. In some embodiments of the pharmaceutical composition comprising an anti-CD30L provided herein, including in Section 2 and this Section 4.5 (such as those of this paragraph), the CDR-L3 of the anti-CD30L comprises the amino acid sequence set forth in any one of SEQ ID NOs: 385-389, 698, 705, 395-399, 699, 706, 618-622, 700, 707, 603-607, 701, 708, 608-612, 702, 709, 613-617, 703, 710, 623-627, 704, and 711. In certain embodiments of the pharmaceutical composition comprising an anti-CD30L provided herein, including in Section 2 and this Section 4.5 (such as those of this paragraph), the CDR-H1 of the anti-CD30L comprises the amino acid sequence set forth in any one of SEQ ID NOs: 105-109, 628, 635, 115-119, 629, 636, 480-484, 630, and 637. In certain embodiments of the pharmaceutical composition comprising an anti-CD30L provided herein, including in Section 2 and this Section 4.5 (such as those of this paragraph), the CDR-H2 of the anti-CD30L comprises the amino acid sequence set forth in any one of SEQ ID NOs: 145-149, 642, 649, 155-159, 643, 650, 518-522, 644, and 651. In certain embodiments of the pharmaceutical composition comprising an anti-CD30L provided herein, including in Section 2 and this Section 4.5 (such as those of this paragraph), the CDR-H3 of the anti-CD30L comprises the amino acid sequence set forth in any one of SEQ ID NOs: 185-189, 656, 663, 195-199, 657, 664, 543-547, 658, and 665. In certain embodiments of the pharmaceutical composition comprising an anti-CD30L provided herein, including in Section 2 and this Section 4.5 (such as those of this paragraph), the CDR-L1 of the anti-CD30L comprises the amino acid sequence set forth in any one of SEQ ID NOs: 305-309, 670, 677, 315-319, 671, 678, 568-572, 672, and 679. In certain embodiments of the pharmaceutical composition comprising an anti-CD30L provided herein, including in Section 2 and this Section 4.5 (such as those of this paragraph), the CDR-L2 of the anti-CD30L comprises the amino acid sequence set forth in any one of SEQ ID NOs: 345-349, 684, 691, 355-359, 685, 692, 593-597, 686, and 693. In certain embodiments of the pharmaceutical composition comprising an anti-CD30L provided herein, including in Section 2 and this Section 4.5 (such as those of this paragraph), the CDR-L3 of the anti-CD30L comprises the amino acid sequence set forth in any one of SEQ ID NOs: 385-389, 698, 705, 395-399, 699, 706, 618-622, 700, and 707.

Furthermore, in one embodiment, the pharmaceutical composition provided herein comprises an anti-CD30L, wherein the anti-CD30L comprises a CDR-H1 comprising the amino acid sequence set forth in any one of SEQ ID NOs: 712-723. In some embodiments, the pharmaceutical composition provided herein comprises an anti-CD30L, wherein the anti-CD30L comprises a CDR-H2 comprising the amino acid sequence set forth in any one of SEQ ID NOs: 724-735. In certain embodiments, the pharmaceutical composition provided herein comprises an anti-CD30L, wherein the anti-CD30L comprises a CDR-H3 comprising the amino acid sequence set forth in any one of SEQ ID NOs: 736-743. In other embodiments, the pharmaceutical composition provided herein comprises an anti-CD30L, wherein the anti-CD30L comprises a CDR-L1 comprising the amino acid sequence set forth in any one of SEQ ID NOs: 744-751. In yet other embodiments, the pharmaceutical composition provided herein comprises an anti-CD30L, wherein the anti-CD30L comprises a CDR-L2 comprising the amino acid sequence set forth in any one of SEQ ID NOs: 752-759. In another embodiment, the pharmaceutical composition provided herein comprises an anti-CD30L, wherein the anti-CD30L comprises a CDR-L3 comprising the amino acid sequence set forth in any one of SEQ ID NOs: 760-765. In one embodiment, the pharmaceutical composition provided herein comprises an anti-CD30L, wherein the anti-CD30L comprises a CDR-H1 comprising the amino acid sequence set forth in any one of SEQ ID NOs: 712-723 and a CDR-H2 comprising the amino acid sequence set forth in any one of SEQ ID NOs: 724-735. In some embodiments, the pharmaceutical composition provided herein comprises an anti-CD30L, wherein the anti-CD30L comprises a CDR-H1 comprising the amino acid sequence set forth in any one of SEQ ID NOs: 712-723 and a CDR-H3 comprising the amino acid sequence set forth in any one of SEQ ID NOs: 736-743. In certain embodiments, the pharmaceutical composition provided herein comprises an anti-CD30L, wherein the anti-CD30L comprises a CDR-H1 comprising the amino acid sequence set forth in any one of SEQ ID NOs: 712-723 and a CDR-L1 comprising the amino acid sequence set forth in any one of SEQ ID NOs: 744-751. In other embodiments, the pharmaceutical composition provided herein comprises an anti-CD30L, wherein the anti-CD30L comprises a CDR-H1 comprising the amino acid sequence set forth in any one of SEQ ID NOs: 712-723 and a CDR-L2 comprising the amino acid sequence set forth in any one of SEQ ID NOs: 752-759. In some embodiments, the pharmaceutical composition provided herein comprises an anti-CD30L, wherein the anti-CD30L comprises a CDR-H1 comprising the amino acid sequence set forth in any one of SEQ ID NOs: 712-723 and a CDR-L3 comprising the amino acid sequence set forth in any one of SEQ ID NOs: 760-765. In further embodiments, the pharmaceutical composition provided herein comprises an anti-CD30L, wherein the anti-CD30L comprises a CDR-H2 comprising the amino acid sequence set forth in any one of SEQ ID NOs: 724-735 and a CDR-H3 comprising the amino acid sequence set forth in any one of SEQ ID NOs: 736-743. In one embodiment, the pharmaceutical composition provided herein comprises an anti-CD30L, wherein the anti-CD30L comprises a CDR-H2 comprising the amino acid sequence set forth in any one of SEQ ID NOs: 724-735 and a CDR-L1 comprising the amino acid sequence set forth in any one of SEQ ID NOs: 744-751. In some embodiments, the pharmaceutical composition provided herein comprises an anti-CD30L, wherein the anti-CD30L comprises a CDR-H2 comprising the amino acid sequence set forth in any one of SEQ ID NOs: 724-735 and a CDR-L2 comprising the amino acid sequence set forth in any one of SEQ ID NOs: 752-759. In certain embodiments, the pharmaceutical composition provided herein comprises an anti-CD30L, wherein the anti-CD30L comprises a CDR-H2 comprising the amino acid sequence set forth in any one of SEQ ID NOs: 724-735 and a CDR-L3 comprising the amino acid sequence set forth in any one of SEQ ID NOs: 760-765. In other embodiments, the pharmaceutical composition provided herein comprises an anti-CD30L, wherein the anti-CD30L comprises a CDR-H3 comprising the amino acid sequence set forth in any one of SEQ ID NOs: 736-743 and a CDR-L1 comprising the amino acid sequence set forth in any one of SEQ ID NOs: 744-751. In yet other embodiments, the pharmaceutical composition provided herein comprises an anti-CD30L, wherein the anti-CD30L comprises a CDR-H3 comprising the amino acid sequence set forth in any one of SEQ ID NOs: 736-743 and a CDR-L2 comprising the amino acid sequence set forth in any one of SEQ ID NOs: 752-759. In some embodiments, the pharmaceutical composition provided herein comprises an anti-CD30L, wherein the anti-CD30L comprises a CDR-H3 comprising the amino acid sequence set forth in any one of SEQ ID NOs: 736-743 and a CDR-L3 comprising the amino acid sequence set forth in any one of SEQ ID NOs: 760-765. In one embodiment, the pharmaceutical composition provided herein comprises an anti-CD30L, wherein the anti-CD30L comprises a CDR-L1 comprising the amino acid sequence set forth in any one of SEQ ID NOs: 744-751 and a CDR-L2 comprising the amino acid sequence set forth in any one of SEQ ID NOs: 752-759. In another embodiment, the pharmaceutical composition provided herein comprises an anti-CD30L, wherein the anti-CD30L comprises a CDR-L1 comprising the amino acid sequence set forth in any one of SEQ ID NOs: 744-751 and a CDR-L3 comprising the amino acid sequence set forth in any one of SEQ ID NOs: 760-765. In another embodiment, the pharmaceutical composition provided herein comprises an anti-CD30L, wherein the anti-CD30L comprises a CDR-L2 comprising the amino acid sequence set forth in any one of SEQ ID NOs: 752-759 and a CDR-L3 comprising the amino acid sequence set forth in any one of SEQ ID NOs: 760-765. In other embodiments, the pharmaceutical composition provided herein comprises an anti-CD30L, wherein the anti-CD30L comprises a CDR-H1 comprising the amino acid sequence set forth in any one of SEQ ID NOs: 712-723, a CDR-H2 comprising the amino acid sequence set forth in any one of SEQ ID NOs: 724-735, and a CDR-H3 comprising the amino acid sequence set forth in any one of SEQ ID NOs: 736-743. In yet other embodiments, the pharmaceutical composition provided herein comprises an anti-CD30L, wherein the anti-CD30L comprises a CDR-H1 comprising the amino acid sequence set forth in any one of SEQ ID NOs: 712-723, a CDR-H2 comprising the amino acid sequence set forth in any one of SEQ ID NOs: 724-735 and a CDR-L1 comprising the amino acid sequence set forth in any one of SEQ ID NOs: 744-751. In further embodiments, the pharmaceutical composition provided herein comprises an anti-CD30L, wherein the anti-CD30L comprises a CDR-H1 comprising the amino acid sequence set forth in any one of SEQ ID NOs: 712-723, a CDR-H2 comprising the amino acid sequence set forth in any one of SEQ ID NOs: 724-735, and a CDR-L2 comprising the amino acid sequence set forth in any one of SEQ ID NOs: 752-759. In some embodiments, the pharmaceutical composition provided herein comprises an anti-CD30L, wherein the anti-CD30L comprises a CDR-H1 comprising the amino acid sequence set forth in any one of SEQ ID NOs: 712-723, a CDR-H2 comprising the amino acid sequence set forth in any one of SEQ ID NOs: 724-735, and a CDR-L3 comprising the amino acid sequence set forth in any one of SEQ ID NOs: 760-765. In some embodiments, the pharmaceutical composition provided herein comprises an anti-CD30L, wherein the anti-CD30L comprises a CDR-H1 comprising the amino acid sequence set forth in any one of SEQ ID NOs: 712-723, a CDR-H3 comprising the amino acid sequence set forth in any one of SEQ ID NOs: 736-743 and a CDR-L1 comprising the amino acid sequence set forth in any one of SEQ ID NOs: 744-751. In certain embodiments, the pharmaceutical composition provided herein comprises an anti-CD30L, wherein the anti-CD30L comprises a CDR-H1 comprising the amino acid sequence set forth in any one of SEQ ID NOs: 712-723, a CDR-H3 comprising the amino acid sequence set forth in any one of SEQ ID NOs: 736-743, and a CDR-L2 comprising the amino acid sequence set forth in any one of SEQ ID NOs: 752-759. In some embodiments, the pharmaceutical composition provided herein comprises an anti-CD30L, wherein the anti-CD30L comprises a CDR-H1 comprising the amino acid sequence set forth in any one of SEQ ID NOs: 712-723, a CDR-H3 comprising the amino acid sequence set forth in any one of SEQ ID NOs: 736-743, and a CDR-L3 comprising the amino acid sequence set forth in any one of SEQ ID NOs: 760-765. In yet other embodiments, the pharmaceutical composition provided herein comprises an anti-CD30L, wherein the anti-CD30L comprises a CDR-H1 comprising the amino acid sequence set forth in any one of SEQ ID NOs: 712-723, a CDR-L1 comprising the amino acid sequence set forth in any one of SEQ ID NOs: 744-751 and a CDR-L2 comprising the amino acid sequence set forth in any one of SEQ ID NOs: 752-759. In some embodiments, the pharmaceutical composition provided herein comprises an anti-CD30L, wherein the anti-CD30L comprises a CDR-H1 comprising the amino acid sequence set forth in any one of SEQ ID NOs: 712-723, a CDR-L1 comprising the amino acid sequence set forth in any one of SEQ ID NOs: 744-751 and a CDR-L3 comprising the amino acid sequence set forth in any one of SEQ ID NOs: 760-765. In some embodiments, the pharmaceutical composition provided herein comprises an anti-CD30L, wherein the anti-CD30L comprises a CDR-H1 comprising the amino acid sequence set forth in any one of SEQ ID NOs: 712-723, a CDR-L2 comprising the amino acid sequence set forth in any one of SEQ ID NOs: 752-759 and a CDR-L3 comprising the amino acid sequence set forth in any one of SEQ ID NOs: 760-765. In some embodiments, the pharmaceutical composition provided herein comprises an anti-CD30L, wherein the anti-CD30L comprises a CDR-H2 comprising the amino acid sequence set forth in any one of SEQ ID NOs: 724-735, a CDR-H3 comprising the amino acid sequence set forth in any one of SEQ ID NOs: 736-743 and a CDR-L1 comprising the amino acid sequence set forth in any one of SEQ ID NOs: 744-751. In certain embodiments, the pharmaceutical composition provided herein comprises an anti-CD30L, wherein the anti-CD30L comprises a CDR-H2 comprising the amino acid sequence set forth in any one of SEQ ID NOs: 724-735, a CDR-H3 comprising the amino acid sequence set forth in any one of SEQ ID NOs: 736-743, and a CDR-L2 comprising the amino acid sequence set forth in any one of SEQ ID NOs: 752-759. In some embodiments, the pharmaceutical composition provided herein comprises an anti-CD30L, wherein the anti-CD30L comprises a CDR-H2 comprising the amino acid sequence set forth in any one of SEQ ID NOs: 724-735, a CDR-H3 comprising the amino acid sequence set forth in any one of SEQ ID NOs: 736-743, and a CDR-L3 comprising the amino acid sequence set forth in any one of SEQ ID NOs: 760-765. In other embodiments, the pharmaceutical composition provided herein comprises an anti-CD30L, wherein the anti-CD30L comprises a CDR-H2 comprising the amino acid sequence set forth in any one of SEQ ID NOs: 724-735, a CDR-L1 comprising the amino acid sequence set forth in any one of SEQ ID NOs: 744-751 and a CDR-L2 comprising the amino acid sequence set forth in any one of SEQ ID NOs: 752-759. In yet other embodiments, the pharmaceutical composition provided herein comprises an anti-CD30L, wherein the anti-CD30L comprises a CDR-H2 comprising the amino acid sequence set forth in any one of SEQ ID NOs: 724-735, a CDR-L1 comprising the amino acid sequence set forth in any one of SEQ ID NOs: 744-751 and a CDR-L3 comprising the amino acid sequence set forth in any one of SEQ ID NOs: 760-765. In some embodiments, the pharmaceutical composition provided herein comprises an anti-CD30L, wherein the anti-CD30L comprises a CDR-H2 comprising the amino acid sequence set forth in any one of SEQ ID NOs: 724-735, a CDR-L2 comprising the amino acid sequence set forth in any one of SEQ ID NOs: 752-759 and a CDR-L3 comprising the amino acid sequence set forth in any one of SEQ ID NOs: 760-765. In yet other embodiments, the pharmaceutical composition provided herein comprises an anti-CD30L, wherein the anti-CD30L comprises a CDR-H3 comprising the amino acid sequence set forth in any one of SEQ ID NOs: 736-743, a CDR-L1 comprising the amino acid sequence set forth in any one of SEQ ID NOs: 744-751 and a CDR-L2 comprising the amino acid sequence set forth in any one of SEQ ID NOs: 752-759. In other embodiments, the pharmaceutical composition provided herein comprises an anti-CD30L, wherein the anti-CD30L comprises a CDR-H3 comprising the amino acid sequence set forth in any one of SEQ ID NOs: 736-743, a CDR-L1 comprising the amino acid sequence set forth in any one of SEQ ID NOs: 744-751 and a CDR-L3 comprising the amino acid sequence set forth in any one of SEQ ID NOs: 760-765. In certain embodiments, the pharmaceutical composition provided herein comprises an anti-CD30L, wherein the anti-CD30L comprises a CDR-H3 comprising the amino acid sequence set forth in any one of SEQ ID NOs: 736-743, a CDR-L2 comprising the amino acid sequence set forth in any one of SEQ ID NOs: 752-759 and a CDR-L3 comprising the amino acid sequence set forth in any one of SEQ ID NOs: 760-765. In some embodiments, the pharmaceutical composition provided herein comprises an anti-CD30L, wherein the anti-CD30L comprises a CDR-L1 comprising the amino acid sequence set forth in any one of SEQ ID NOs: 744-751, a CDR-L2 comprising the amino acid sequence set forth in any one of SEQ ID NOs: 752-759 and a CDR-L3 comprising the amino acid sequence set forth in any one of SEQ ID NOs: 760-765. In other embodiments, the pharmaceutical composition provided herein comprises an anti-CD30L, wherein the anti-CD30L comprises a CDR-H1 comprising the amino acid sequence set forth in any one of SEQ ID NOs: 712-723, a CDR-H2 comprising the amino acid sequence set forth in any one of SEQ ID NOs: 724-735, a CDR-H3 comprising the amino acid sequence set forth in any one of SEQ ID NOs: 736-743, and a CDR-L1 comprising the amino acid sequence set forth in any one of SEQ ID NOs: 744-751. In further embodiments, the pharmaceutical composition provided herein comprises an anti-CD30L, wherein the anti-CD30L comprises a CDR-H1 comprising the amino acid sequence set forth in any one of SEQ ID NOs: 712-723, a CDR-H2 comprising the amino acid sequence set forth in any one of SEQ ID NOs: 724-735, a CDR-H3 comprising the amino acid sequence set forth in any one of SEQ ID NOs: 736-743, and a CDR-L2 comprising the amino acid sequence set forth in any one of SEQ ID NOs: 752-759. In some embodiments, the pharmaceutical composition provided herein comprises an anti-CD30L, wherein the anti-CD30L comprises a CDR-H1 comprising the amino acid sequence set forth in any one of SEQ ID NOs: 712-723, a CDR-H2 comprising the amino acid sequence set forth in any one of SEQ ID NOs: 724-735, a CDR-H3 comprising the amino acid sequence set forth in any one of SEQ ID NOs: 736-743, and a CDR-L3 comprising the amino acid sequence set forth in any one of SEQ ID NOs: 760-765. In some embodiments, the pharmaceutical composition provided herein comprises an anti-CD30L, wherein the anti-CD30L comprises a CDR-H1 comprising the amino acid sequence set forth in any one of SEQ ID NOs: 712-723, a CDR-H2 comprising the amino acid sequence set forth in any one of SEQ ID NOs: 724-735, a CDR-L1 comprising the amino acid sequence set forth in any one of SEQ ID NOs: 744-751, and a CDR-L2 comprising the amino acid sequence set forth in any one of SEQ ID NOs: 752-759. In some embodiments, the pharmaceutical composition provided herein comprises an anti-CD30L, wherein the anti-CD30L comprises a CDR-H1 comprising the amino acid sequence set forth in any one of SEQ ID NOs: 712-723, a CDR-H2 comprising the amino acid sequence set forth in any one of SEQ ID NOs: 724-735, a CDR-L1 comprising the amino acid sequence set forth in any one of SEQ ID NOs: 744-751, and a CDR-L3 comprising the amino acid sequence set forth in any one of SEQ ID NOs: 760-765. In some embodiments, the pharmaceutical composition provided herein comprises an anti-CD30L, wherein the anti-CD30L comprises a CDR-H1 comprising the amino acid sequence set forth in any one of SEQ ID NOs: 712-723, a CDR-H2 comprising the amino acid sequence set forth in any one of SEQ ID NOs: 724-735, a CDR-L2 comprising the amino acid sequence set forth in any one of SEQ ID NOs: 752-759, and a CDR-L3 comprising the amino acid sequence set forth in any one of SEQ ID NOs: 760-765. In certain embodiments, the pharmaceutical composition provided herein comprises an anti-CD30L, wherein the anti-CD30L comprises a CDR-H1 comprising the amino acid sequence set forth in any one of SEQ ID NOs: 712-723, a CDR-H3 comprising the amino acid sequence set forth in any one of SEQ ID NOs: 736-743, a CDR-L1 comprising the amino acid sequence set forth in any one of SEQ ID NOs: 744-751, and a CDR-L2 comprising the amino acid sequence set forth in any one of SEQ ID NOs: 752-759. In certain embodiments, the pharmaceutical composition provided herein comprises an anti-CD30L, wherein the anti-CD30L comprises a CDR-H1 comprising the amino acid sequence set forth in any one of SEQ ID NOs: 712-723, a CDR-H3 comprising the amino acid sequence set forth in any one of SEQ ID NOs: 736-743, a CDR-L1 comprising the amino acid sequence set forth in any one of SEQ ID NOs: 744-751, and a CDR-L3 comprising the amino acid sequence set forth in any one of SEQ ID NOs: 760-765. In certain embodiments, the pharmaceutical composition provided herein comprises an anti-CD30L, wherein the anti-CD30L comprises a CDR-H1 comprising the amino acid sequence set forth in any one of SEQ ID NOs: 712-723, a CDR-H3 comprising the amino acid sequence set forth in any one of SEQ ID NOs: 736-743, a CDR-L2 comprising the amino acid sequence set forth in any one of SEQ ID NOs: 752-759, and a CDR-L3 comprising the amino acid sequence set forth in any one of SEQ ID NOs: 760-765. In yet other embodiments, the pharmaceutical composition provided herein comprises an anti-CD30L, wherein the anti-CD30L comprises a CDR-H1 comprising the amino acid sequence set forth in any one of SEQ ID NOs: 712-723, a CDR-L1 comprising the amino acid sequence set forth in any one of SEQ ID NOs: 744-751, a CDR-L2 comprising the amino acid sequence set forth in any one of SEQ ID NOs: 752-759, and a CDR-L3 comprising the amino acid sequence set forth in any one of SEQ ID NOs: 760-765. In further embodiments, the pharmaceutical composition provided herein comprises an anti-CD30L, wherein the anti-CD30L comprises a CDR-H2 comprising the amino acid sequence set forth in any one of SEQ ID NOs: 724-735, a CDR-H3 comprising the amino acid sequence set forth in any one of SEQ ID NOs: 736-743, a CDR-L1 comprising the amino acid sequence set forth in any one of SEQ ID NOs: 744-751, and a CDR-L2 comprising the amino acid sequence set forth in any one of SEQ ID NOs: 752-759. In some embodiments, the pharmaceutical composition provided herein comprises an anti-CD30L, wherein the anti-CD30L comprises a CDR-H2 comprising the amino acid sequence set forth in any one of SEQ ID NOs: 724-735, a CDR-H3 comprising the amino acid sequence set forth in any one of SEQ ID NOs: 736-743, a CDR-L1 comprising the amino acid sequence set forth in any one of SEQ ID NOs: 744-751, and a CDR-L3 comprising the amino acid sequence set forth in any one of SEQ ID NOs: 760-765. In further embodiments, the pharmaceutical composition provided herein comprises an anti-CD30L, wherein the anti-CD30L comprises a CDR-H2 comprising the amino acid sequence set forth in any one of SEQ ID NOs: 724-735, a CDR-H3 comprising the amino acid sequence set forth in any one of SEQ ID NOs: 736-743, a CDR-L2 comprising the amino acid sequence set forth in any one of SEQ ID NOs: 752-759, and a CDR-L3 comprising the amino acid sequence set forth in any one of SEQ ID NOs: 760-765. In some embodiments, the pharmaceutical composition provided herein comprises an anti-CD30L, wherein the anti-CD30L comprises a CDR-H2 comprising the amino acid sequence set forth in any one of SEQ ID NOs: 724-735, a CDR-L1 comprising the amino acid sequence set forth in any one of SEQ ID NOs: 744-751, a CDR-L2 comprising the amino acid sequence set forth in any one of SEQ ID NOs: 752-759, and a CDR-L3 comprising the amino acid sequence set forth in any one of SEQ ID NOs: 760-765. In some embodiments, the pharmaceutical composition provided herein comprises an anti-CD30L, wherein the anti-CD30L comprises a CDR-H3 comprising the amino acid sequence set forth in any one of SEQ ID NOs: 736-743, a CDR-L1 comprising the amino acid sequence set forth in any one of SEQ ID NOs: 744-751, a CDR-L2 comprising the amino acid sequence set forth in any one of SEQ ID NOs: 752-759, and a CDR-L3 comprising the amino acid sequence set forth in any one of SEQ ID NOs: 760-765. In certain embodiments, the pharmaceutical composition provided herein comprises an anti-CD30L, wherein the anti-CD30L comprises a CDR-H1 comprising the amino acid sequence set forth in any one of SEQ ID NOs: 712-723, a CDR-H2 comprising the amino acid sequence set forth in any one of SEQ ID NOs: 724-735, a CDR-H3 comprising the amino acid sequence set forth in any one of SEQ ID NOs: 736-743, a CDR-L1 comprising the amino acid sequence set forth in any one of SEQ ID NOs: 744-751, and a CDR-L2 comprising the amino acid sequence set forth in any one of SEQ ID NOs: 752-759. In some embodiments, the pharmaceutical composition provided herein comprises an anti-CD30L, wherein the anti-CD30L comprises a CDR-H1 comprising the amino acid sequence set forth in any one of SEQ ID NOs: 712-723, a CDR-H2 comprising the amino acid sequence set forth in any one of SEQ ID NOs: 724-735, a CDR-H3 comprising the amino acid sequence set forth in any one of SEQ ID NOs: 736-743, a CDR-L1 comprising the amino acid sequence set forth in any one of SEQ ID NOs: 744-751, and a CDR-L3 comprising the amino acid sequence set forth in any one of SEQ ID NOs: 760-765. In some embodiments, the pharmaceutical composition provided herein comprises an anti-CD30L, wherein the anti-CD30L comprises a CDR-H1 comprising the amino acid sequence set forth in any one of SEQ ID NOs: 712-723, a CDR-H2 comprising the amino acid sequence set forth in any one of SEQ ID NOs: 724-735, a CDR-H3 comprising the amino acid sequence set forth in any one of SEQ ID NOs: 736-743, a CDR-L2 comprising the amino acid sequence set forth in any one of SEQ ID NOs: 752-759, and a CDR-L3 comprising the amino acid sequence set forth in any one of SEQ ID NOs: 760-765. In certain embodiments, the pharmaceutical composition provided herein comprises an anti-CD30L, wherein the anti-CD30L comprises a CDR-H1 comprising the amino acid sequence set forth in any one of SEQ ID NOs: 712-723, a CDR-H2 comprising the amino acid sequence set forth in any one of SEQ ID NOs: 724-735, a CDR-L1 comprising the amino acid sequence set forth in any one of SEQ ID NOs: 744-751, a CDR-L2 comprising the amino acid sequence set forth in any one of SEQ ID NOs: 752-759, and a CDR-L3 comprising the amino acid sequence set forth in any one of SEQ ID NOs: 760-765. In certain embodiments, the pharmaceutical composition provided herein comprises an anti-CD30L, wherein the anti-CD30L comprises a CDR-H1 comprising the amino acid sequence set forth in any one of SEQ ID NOs: 712-723, a CDR-H3 comprising the amino acid sequence set forth in any one of SEQ ID NOs: 736-743, a CDR-L1 comprising the amino acid sequence set forth in any one of SEQ ID NOs: 744-751, a CDR-L2 comprising the amino acid sequence set forth in any one of SEQ ID NOs: 752-759, and a CDR-L3 comprising the amino acid sequence set forth in any one of SEQ ID NOs: 760-765. In some embodiments, the pharmaceutical composition provided herein comprises an anti-CD30L, wherein the anti-CD30L comprises a CDR-H2 comprising the amino acid sequence set forth in any one of SEQ ID NOs: 724-735, a CDR-H3 comprising the amino acid sequence set forth in any one of SEQ ID NOs: 736-743, a CDR-L1 comprising the amino acid sequence set forth in any one of SEQ ID NOs: 744-751, a CDR-L2 comprising the amino acid sequence set forth in any one of SEQ ID NOs: 752-759, and a CDR-L3 comprising the amino acid sequence set forth in any one of SEQ ID NOs: 760-765. In some embodiments, the pharmaceutical composition provided herein comprises an anti-CD30L, wherein the anti-CD30L comprises a CDR-H1 comprising the amino acid sequence set forth in any one of SEQ ID NOs: 712-723, a CDR-H2 comprising the amino acid sequence set forth in any one of SEQ ID NOs: 724-735, a CDR-H3 comprising the amino acid sequence set forth in any one of SEQ ID NOs: 736-743, a CDR-L1 comprising the amino acid sequence set forth in any one of SEQ ID NOs: 744-751, a CDR-L2 comprising the amino acid sequence set forth in any one of SEQ ID NOs: 752-759, and a CDR-L3 comprising the amino acid sequence set forth in any one of SEQ ID NOs: 760-765. In some embodiments, the pharmaceutical composition provided herein comprises an anti-CD30L, wherein the anti-CD30L comprises any one of a CDR-H1 comprising the amino acid sequence set forth in any one of SEQ ID NOs: 712-723, a CDR-H2 comprising the amino acid sequence set forth in any one of SEQ ID NOs: 724-735, a CDR-H3 comprising the amino acid sequence set forth in any one of SEQ ID NOs: 736-743, a CDR-L1 comprising the amino acid sequence set forth in any one of SEQ ID NOs: 744-751, a CDR-L2 comprising the amino acid sequence set forth in any one of SEQ ID NOs: 752-759, and a CDR-L3 comprising the amino acid sequence set forth in any one of SEQ ID NOs: 760-765. In some embodiments, the pharmaceutical composition provided herein comprises an anti-CD30L, wherein the anti-CD30L comprises any two of a CDR-H1 comprising the amino acid sequence set forth in any one of SEQ ID NOs: 712-723, a CDR-H2 comprising the amino acid sequence set forth in any one of SEQ ID NOs: 724-735, a CDR-H3 comprising the amino acid sequence set forth in any one of SEQ ID NOs: 736-743, a CDR-L1 comprising the amino acid sequence set forth in any one of SEQ ID NOs: 744-751, a CDR-L2 comprising the amino acid sequence set forth in any one of SEQ ID NOs: 752-759, and a CDR-L3 comprising the amino acid sequence set forth in any one of SEQ ID NOs: 760-765, in any combination or permutation. In some embodiments, the pharmaceutical composition provided herein comprises an anti-CD30L, wherein the anti-CD30L comprises any three of a CDR-H1 comprising the amino acid sequence set forth in any one of SEQ ID NOs: 712-723, a CDR-H2 comprising the amino acid sequence set forth in any one of SEQ ID NOs: 724-735, a CDR-H3 comprising the amino acid sequence set forth in any one of SEQ ID NOs: 736-743, a CDR-L1 comprising the amino acid sequence set forth in any one of SEQ ID NOs: 744-751, a CDR-L2 comprising the amino acid sequence set forth in any one of SEQ ID NOs: 752-759, and a CDR-L3 comprising the amino acid sequence set forth in any one of SEQ ID NOs: 760-765, in any combination or permutation. In some embodiments, the pharmaceutical composition provided herein comprises an anti-CD30L, wherein the anti-CD30L comprises any four of a CDR-H1 comprising the amino acid sequence set forth in any one of SEQ ID NOs: 712-723, a CDR-H2 comprising the amino acid sequence set forth in any one of SEQ ID NOs: 724-735, a CDR-H3 comprising the amino acid sequence set forth in any one of SEQ ID NOs: 736-743, a CDR-L1 comprising the amino acid sequence set forth in any one of SEQ ID NOs: 744-751, a CDR-L2 comprising the amino acid sequence set forth in any one of SEQ ID NOs: 752-759, and a CDR-L3 comprising the amino acid sequence set forth in any one of SEQ ID NOs: 760-765, in any combination or permutation. In some embodiments, the pharmaceutical composition provided herein comprises an anti-CD30L, wherein the anti-CD30L comprises any five of a CDR-H1 comprising the amino acid sequence set forth in any one of SEQ ID NOs: 712-723, a CDR-H2 comprising the amino acid sequence set forth in any one of SEQ ID NOs: 724-735, a CDR-H3 comprising the amino acid sequence set forth in any one of SEQ ID NOs: 736-743, a CDR-L1 comprising the amino acid sequence set forth in any one of SEQ ID NOs: 744-751, a CDR-L2 comprising the amino acid sequence set forth in any one of SEQ ID NOs: 752-759, and a CDR-L3 comprising the amino acid sequence set forth in any one of SEQ ID NOs: 760-765, in any combination or permutation. In some embodiments, the pharmaceutical composition provided herein comprises an anti-CD30L, wherein the anti-CD30L comprises all six of a CDR-H1 comprising the amino acid sequence set forth in any one of SEQ ID NOs: 712-723, a CDR-H2 comprising the amino acid sequence set forth in any one of SEQ ID NOs: 724-735, a CDR-H3 comprising the amino acid sequence set forth in any one of SEQ ID NOs: 736-743, a CDR-L1 comprising the amino acid sequence set forth in any one of SEQ ID NOs: 744-751, a CDR-L2 comprising the amino acid sequence set forth in any one of SEQ ID NOs: 752-759, and a CDR-L3 comprising the amino acid sequence set forth in any one of SEQ ID NOs: 760-765. In some embodiments of the pharmaceutical composition comprising anti-CD30L provided herein including in Section 2 and this Section 4.5 (such as those of this paragraph), the CDR-H1 of the anti-CD30L comprises the amino acid sequence set forth in any one of SEQ ID NOs: 105-109, 628, 635, 115-119, 629, 636, 480-484, 630, 637, 465-469, 631, 638, 470-474, 632, 639, 475-479, 633, 640, 485-489, 634, and 641. In some embodiments of the pharmaceutical composition comprising anti-CD30L provided herein including in Section 2 and this Section 4.5 (such as those of this paragraph), the CDR-H2 of the anti-CD30L comprises the amino acid sequence set forth in any one of SEQ ID NOs: 145-149, 642, 649, 155-159, 643, 650, 518-522, 644, 651, 490-494, 645, 652, 495-499, 646, 653, 513-517, 647, 654, 523-527, 648, and 655. In some embodiments of the pharmaceutical composition comprising anti-CD30L provided herein including in Section 2 and this Section 4.5 (such as those of this paragraph), the CDR-H3 of the anti-CD30L comprises the amino acid sequence set forth in any one of SEQ ID NOs: 185-189, 656, 663, 195-199, 657, 664, 543-547, 658, 665, 528-532, 659, 666, 533-537, 660, 667, 538-542, 661, 668, 548-552, 662, and 669. In some embodiments of the pharmaceutical composition comprising anti-CD30L provided herein including in Section 2 and this Section 4.5 (such as those of this paragraph), the CDR-L1 of the anti-CD30L comprises the amino acid sequence set forth in any one of SEQ ID NOs: 305-309, 670, 677, 315-319, 671, 678, 568-572, 672, 679, 553-557, 673, 680, 558-562, 674, 681, 563-567, 675, 682, 573-577, 676, and 683. In some embodiments of the pharmaceutical composition comprising anti-CD30L provided herein including in Section 2 and this Section 4.5 (such as those of this paragraph), the CDR-L2 of the anti-CD30L comprises the amino acid sequence set forth in any one of SEQ ID NOs: 345-349, 684, 691, 355-359, 685, 692, 593-597, 686, 693, 578-582, 687, 694, 583-587, 688, 695, 588-592, 689, 696, 598-602, 690, and 697. In some embodiments of the pharmaceutical composition comprising anti-CD30L provided herein including in Section 2 and this Section 4.5 (such as those of this paragraph), the CDR-L3 of the anti-CD30L comprises the amino acid sequence set forth in any one of SEQ ID NOs: 385-389, 698, 705, 395-399, 699, 706, 618-622, 700, 707, 603-607, 701, 708, 608-612, 702, 709, 613-617, 703, 710, 623-627, 704, and 711. In certain embodiments of the pharmaceutical composition comprising anti-CD30L provided herein including in Section 2 and this Section 4.5 (such as those of this paragraph), the CDR-H1 of the anti-CD30L comprises the amino acid sequence set forth in any one of SEQ ID NOs: 105-109, 628, 635, 115-119, 629, 636, 480-484, 630, and 637. In certain embodiments of the pharmaceutical composition comprising anti-CD30L provided herein including in Section 2 and this Section 4.5 (such as those of this paragraph), the CDR-H2 of the anti-CD30L comprises the amino acid sequence set forth in any one of SEQ ID NOs: 145-149, 642, 649, 155-159, 643, 650, 518-522, 644, and 651. In certain embodiments of the pharmaceutical composition comprising anti-CD30L provided herein including in Section 2 and this Section 4.5 (such as those of this paragraph), the CDR-H3 of the anti-CD30L comprises the amino acid sequence set forth in any one of SEQ ID NOs: 185-189, 656, 663, 195-199, 657, 664, 543-547, 658, and 665. In certain embodiments of the pharmaceutical composition comprising anti-CD30L provided herein including in Section 2 and this Section 4.5 (such as those of this paragraph), the CDR-L1 of the anti-CD30L comprises the amino acid sequence set forth in any one of SEQ ID NOs: 305-309, 670, 677, 315-319, 671, 678, 568-572, 672, and 679. In certain embodiments of the pharmaceutical composition comprising anti-CD30L provided herein including in Section 2 and this Section 4.5 (such as those of this paragraph), the CDR-L2 of the anti-CD30L comprises the amino acid sequence set forth in any one of SEQ ID NOs: 345-349, 684, 691, 355-359, 685, 692, 593-597, 686, and 693. In certain embodiments of the pharmaceutical composition comprising anti-CD30L provided herein including in Section 2 and this Section 4.5 (such as those of this paragraph), the CDR-L3 of the anti-CD30L comprises the amino acid sequence set forth in any one of SEQ ID NOs: 385-389, 698, 705, 395-399, 699, 706, 618-622, 700, and 707.

Additionally, the pharmaceutical composition comprising anti-CD30L provided herein including in Section 2 and this Section 4.5, can comprise an anti-CD30L comprising specific combinations of CDRs of each anti-CD30L clone as listed in Tables 10-16. Accordingly, in one embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 635; (b) a CDR-H2 comprising the amino acid sequence set forth in SEQ ID NO: 649; (c) a CDR-H3 comprising the amino acid sequence set forth in SEQ ID NO: 663; (d) a CDR-L1 comprising the amino acid sequence set forth in SEQ ID NO: 677; (e) a CDR-L2 comprising the amino acid sequence set forth SEQ ID NO: 691; and/or (f) a CDR-L3 comprising the amino acid sequence set forth SEQ ID NO: 705. In another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 107; (b) a CDR-H2 comprising the amino acid sequence set forth in SEQ ID NO: 147; (c) a CDR-H3 comprising the amino acid sequence set forth in SEQ ID NO: 187; (d) a CDR-L1 comprising the amino acid sequence set forth in SEQ ID NO: 307; (e) a CDR-L2 comprising the amino acid sequence set forth SEQ ID NO: 347; and/or (f) a CDR-L3 comprising the amino acid sequence set forth SEQ ID NO: 387. In one embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 105; (b) a CDR-H2 comprising the amino acid sequence set forth in SEQ ID NO: 145; (c) a CDR-H3 comprising the amino acid sequence set forth in SEQ ID NO: 185; (d) a CDR-L1 comprising the amino acid sequence set forth in SEQ ID NO: 305; (e) a CDR-L2 comprising the amino acid sequence set forth SEQ ID NO: 345; and/or (f) a CDR-L3 comprising the amino acid sequence set forth SEQ ID NO: 385. In one embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 106; (b) a CDR-H2 comprising the amino acid sequence set forth in SEQ ID NO: 146; (c) a CDR-H3 comprising the amino acid sequence set forth in SEQ ID NO: 186; (d) a CDR-L1 comprising the amino acid sequence set forth in SEQ ID NO: 306; (e) a CDR-L2 comprising the amino acid sequence set forth SEQ ID NO: 346; and/or (f) a CDR-L3 comprising the amino acid sequence set forth SEQ ID NO: 386. In one embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 108; (b) a CDR-H2 comprising the amino acid sequence set forth in SEQ ID NO: 148; (c) a CDR-H3 comprising the amino acid sequence set forth in SEQ ID NO: 188; (d) a CDR-L1 comprising the amino acid sequence set forth in SEQ ID NO: 308; (e) a CDR-L2 comprising the amino acid sequence set forth SEQ ID NO: 348; and/or (f) a CDR-L3 comprising the amino acid sequence set forth SEQ ID NO: 388. In one embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 109; (b) a CDR-H2 comprising the amino acid sequence set forth in SEQ ID NO: 149; (c) a CDR-H3 comprising the amino acid sequence set forth in SEQ ID NO: 189; (d) a CDR-L1 comprising the amino acid sequence set forth in SEQ ID NO: 309; (e) a CDR-L2 comprising the amino acid sequence set forth SEQ ID NO: 349; and/or (f) a CDR-L3 comprising the amino acid sequence set forth SEQ ID NO: 389. In one embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 628; (b) a CDR-H2 comprising the amino acid sequence set forth in SEQ ID NO: 642; (c) a CDR-H3 comprising the amino acid sequence set forth in SEQ ID NO: 656; (d) a CDR-L1 comprising the amino acid sequence set forth in SEQ ID NO: 670; (e) a CDR-L2 comprising the amino acid sequence set forth SEQ ID NO: 684; and/or (f) a CDR-L3 comprising the amino acid sequence set forth SEQ ID NO: 698.

Similarly, in one embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 636; (b) a CDR-H2 comprising the amino acid sequence set forth in SEQ ID NO: 650; (c) a CDR-H3 comprising the amino acid sequence set forth in SEQ ID NO: 664; (d) a CDR-L1 comprising the amino acid sequence set forth in SEQ ID NO: 678; (e) a CDR-L2 comprising the amino acid sequence set forth SEQ ID NO: 692; and/or (f) a CDR-L3 comprising the amino acid sequence set forth SEQ ID NO: 706. In another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 117; (b) a CDR-H2 comprising the amino acid sequence set forth in SEQ ID NO: 157; (c) a CDR-H3 comprising the amino acid sequence set forth in SEQ ID NO: 197; (d) a CDR-L1 comprising the amino acid sequence set forth in SEQ ID NO: 317; (e) a CDR-L2 comprising the amino acid sequence set forth SEQ ID NO: 357; and/or (f) a CDR-L3 comprising the amino acid sequence set forth SEQ ID NO: 397. In yet another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 115; (b) a CDR-H2 comprising the amino acid sequence set forth in SEQ ID NO: 155; (c) a CDR-H3 comprising the amino acid sequence set forth in SEQ ID NO: 195; (d) a CDR-L1 comprising the amino acid sequence set forth in SEQ ID NO: 315; (e) a CDR-L2 comprising the amino acid sequence set forth SEQ ID NO: 355; and/or (f) a CDR-L3 comprising the amino acid sequence set forth SEQ ID NO: 395. In one embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 116; (b) a CDR-H2 comprising the amino acid sequence set forth in SEQ ID NO: 156; (c) a CDR-H3 comprising the amino acid sequence set forth in SEQ ID NO: 196; (d) a CDR-L1 comprising the amino acid sequence set forth in SEQ ID NO: 316; (e) a CDR-L2 comprising the amino acid sequence set forth SEQ ID NO: 356; and/or (f) a CDR-L3 comprising the amino acid sequence set forth SEQ ID NO: 396. In another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 118; (b) a CDR-H2 comprising the amino acid sequence set forth in SEQ ID NO: 158; (c) a CDR-H3 comprising the amino acid sequence set forth in SEQ ID NO: 198; (d) a CDR-L1 comprising the amino acid sequence set forth in SEQ ID NO: 318; (e) a CDR-L2 comprising the amino acid sequence set forth SEQ ID NO: 358; and/or (f) a CDR-L3 comprising the amino acid sequence set forth SEQ ID NO: 398. In yet another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 119; (b) a CDR-H2 comprising the amino acid sequence set forth in SEQ ID NO: 159; (c) a CDR-H3 comprising the amino acid sequence set forth in SEQ ID NO: 199; (d) a CDR-L1 comprising the amino acid sequence set forth in SEQ ID NO: 319; (e) a CDR-L2 comprising the amino acid sequence set forth SEQ ID NO: 359; and/or (f) a CDR-L3 comprising the amino acid sequence set forth SEQ ID NO: 399. In one embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 629; (b) a CDR-H2 comprising the amino acid sequence set forth in SEQ ID NO: 643; (c) a CDR-H3 comprising the amino acid sequence set forth in SEQ ID NO: 657; (d) a CDR-L1 comprising the amino acid sequence set forth in SEQ ID NO: 671; (e) a CDR-L2 comprising the amino acid sequence set forth SEQ ID NO: 685; and/or (f) a CDR-L3 comprising the amino acid sequence set forth SEQ ID NO: 699.

Likewise, in one embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 637; (b) a CDR-H2 comprising the amino acid sequence set forth in SEQ ID NO: 651; (c) a CDR-H3 comprising the amino acid sequence set forth in SEQ ID NO: 665; (d) a CDR-L1 comprising the amino acid sequence set forth in SEQ ID NO: 679; (e) a CDR-L2 comprising the amino acid sequence set forth SEQ ID NO: 693; and/or (f) a CDR-L3 comprising the amino acid sequence set forth SEQ ID NO: 707. In another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 482; (b) a CDR-H2 comprising the amino acid sequence set forth in SEQ ID NO: 520; (c) a CDR-H3 comprising the amino acid sequence set forth in SEQ ID NO: 545; (d) a CDR-L1 comprising the amino acid sequence set forth in SEQ ID NO: 570; (e) a CDR-L2 comprising the amino acid sequence set forth SEQ ID NO: 595; and/or (f) a CDR-L3 comprising the amino acid sequence set forth SEQ ID NO: 620. In yet another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 480; (b) a CDR-H2 comprising the amino acid sequence set forth in SEQ ID NO: 518; (c) a CDR-H3 comprising the amino acid sequence set forth in SEQ ID NO: 543; (d) a CDR-L1 comprising the amino acid sequence set forth in SEQ ID NO: 568; (e) a CDR-L2 comprising the amino acid sequence set forth SEQ ID NO: 593; and/or (f) a CDR-L3 comprising the amino acid sequence set forth SEQ ID NO: 618. In one embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 481; (b) a CDR-H2 comprising the amino acid sequence set forth in SEQ ID NO: 519; (c) a CDR-H3 comprising the amino acid sequence set forth in SEQ ID NO: 544; (d) a CDR-L1 comprising the amino acid sequence set forth in SEQ ID NO: 569; (e) a CDR-L2 comprising the amino acid sequence set forth SEQ ID NO: 594; and/or (f) a CDR-L3 comprising the amino acid sequence set forth SEQ ID NO: 619. In another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 483; (b) a CDR-H2 comprising the amino acid sequence set forth in SEQ ID NO: 521; (c) a CDR-H3 comprising the amino acid sequence set forth in SEQ ID NO: 546; (d) a CDR-L1 comprising the amino acid sequence set forth in SEQ ID NO: 571; (e) a CDR-L2 comprising the amino acid sequence set forth SEQ ID NO: 596; and/or (f) a CDR-L3 comprising the amino acid sequence set forth SEQ ID NO: 621. In yet another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 484; (b) a CDR-H2 comprising the amino acid sequence set forth in SEQ ID NO: 522; (c) a CDR-H3 comprising the amino acid sequence set forth in SEQ ID NO: 547; (d) a CDR-L1 comprising the amino acid sequence set forth in SEQ ID NO: 572; (e) a CDR-L2 comprising the amino acid sequence set forth SEQ ID NO: 597; and/or (f) a CDR-L3 comprising the amino acid sequence set forth SEQ ID NO: 622. In one embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 630; (b) a CDR-H2 comprising the amino acid sequence set forth in SEQ ID NO: 644; (c) a CDR-H3 comprising the amino acid sequence set forth in SEQ ID NO: 658; (d) a CDR-L1 comprising the amino acid sequence set forth in SEQ ID NO: 672; (e) a CDR-L2 comprising the amino acid sequence set forth SEQ ID NO: 686; and/or (f) a CDR-L3 comprising the amino acid sequence set forth SEQ ID NO: 700.

Additionally, in one embodiment of the pharmaceutical composition comprising an anti-CD30L antibody or antigen binding fragment thereof provided herein including in Section 2 and this Section 4.5, the antibody or antigen binding fragment thereof comprises: (i)(a) a CDR-H1 comprising the amino acid sequence set forth in SEQ ID NO: 638; (b) a CDR-H2 comprising the amino acid sequence set forth in SEQ ID NO: 652; (c) a CDR-H3 comprising the amino acid sequence set forth in SEQ ID NO: 666; (d) a CDR-L1 comprising the amino acid sequence set forth in SEQ ID NO: 680; (e) a CDR-L2 comprising the amino acid sequence set forth SEQ ID NO: 694; and/or (f) a CDR-L3 comprising the amino acid sequence set forth SEQ ID NO: 708. In another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 467; (b) a CDR-H2 comprising the amino acid sequence set forth in SEQ ID NO: 492; (c) a CDR-H3 comprising the amino acid sequence set forth in SEQ ID NO: 530; (d) a CDR-L1 comprising the amino acid sequence set forth in SEQ ID NO: 555; (e) a CDR-L2 comprising the amino acid sequence set forth SEQ ID NO: 580; and/or (f) a CDR-L3 comprising the amino acid sequence set forth SEQ ID NO: 605. In yet another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 465; (b) a CDR-H2 comprising the amino acid sequence set forth in SEQ ID NO: 490; (c) a CDR-H3 comprising the amino acid sequence set forth in SEQ ID NO: 528; (d) a CDR-L1 comprising the amino acid sequence set forth in SEQ ID NO: 553; (e) a CDR-L2 comprising the amino acid sequence set forth SEQ ID NO: 578; and/or (f) a CDR-L3 comprising the amino acid sequence set forth SEQ ID NO: 603. In one embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 466; (b) a CDR-H2 comprising the amino acid sequence set forth in SEQ ID NO: 491; (c) a CDR-H3 comprising the amino acid sequence set forth in SEQ ID NO: 529; (d) a CDR-L1 comprising the amino acid sequence set forth in SEQ ID NO: 554; (e) a CDR-L2 comprising the amino acid sequence set forth SEQ ID NO: 579; and/or (f) a CDR-L3 comprising the amino acid sequence set forth SEQ ID NO: 604. In another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 468; (b) a CDR-H2 comprising the amino acid sequence set forth in SEQ ID NO: 493; (c) a CDR-H3 comprising the amino acid sequence set forth in SEQ ID NO: 531; (d) a CDR-L1 comprising the amino acid sequence set forth in SEQ ID NO: 556; (e) a CDR-L2 comprising the amino acid sequence set forth SEQ ID NO: 581; and/or (f) a CDR-L3 comprising the amino acid sequence set forth SEQ ID NO: 606. In yet another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 469; (b) a CDR-H2 comprising the amino acid sequence set forth in SEQ ID NO: 494; (c) a CDR-H3 comprising the amino acid sequence set forth in SEQ ID NO: 532; (d) a CDR-L1 comprising the amino acid sequence set forth in SEQ ID NO: 557; (e) a CDR-L2 comprising the amino acid sequence set forth SEQ ID NO: 582; and/or (f) a CDR-L3 comprising the amino acid sequence set forth SEQ ID NO: 607. In one embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 631; (b) a CDR-H2 comprising the amino acid sequence set forth in SEQ ID NO: 645; (c) a CDR-H3 comprising the amino acid sequence set forth in SEQ ID NO: 659; (d) a CDR-L1 comprising the amino acid sequence set forth in SEQ ID NO: 673; (e) a CDR-L2 comprising the amino acid sequence set forth SEQ ID NO: 687; and/or (f) a CDR-L3 comprising the amino acid sequence set forth SEQ ID NO: 701.

Similarly, in one embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 639; (b) a CDR-H2 comprising the amino acid sequence set forth in SEQ ID NO: 653; (c) a CDR-H3 comprising the amino acid sequence set forth in SEQ ID NO: 667; (d) a CDR-L1 comprising the amino acid sequence set forth in SEQ ID NO: 681; (e) a CDR-L2 comprising the amino acid sequence set forth SEQ ID NO: 695; and/or (f) a CDR-L3 comprising the amino acid sequence set forth SEQ ID NO: 709. In another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 472; (b) a CDR-H2 comprising the amino acid sequence set forth in SEQ ID NO: 497; (c) a CDR-H3 comprising the amino acid sequence set forth in SEQ ID NO: 535; (d) a CDR-L1 comprising the amino acid sequence set forth in SEQ ID NO: 560; (e) a CDR-L2 comprising the amino acid sequence set forth SEQ ID NO: 585; and/or (f) a CDR-L3 comprising the amino acid sequence set forth SEQ ID NO: 610. In yet another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 470; (b) a CDR-H2 comprising the amino acid sequence set forth in SEQ ID NO: 495; (c) a CDR-H3 comprising the amino acid sequence set forth in SEQ ID NO: 533; (d) a CDR-L1 comprising the amino acid sequence set forth in SEQ ID NO: 558; (e) a CDR-L2 comprising the amino acid sequence set forth SEQ ID NO: 583; and/or (f) a CDR-L3 comprising the amino acid sequence set forth SEQ ID NO: 608. In one embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 471; (b) a CDR-H2 comprising the amino acid sequence set forth in SEQ ID NO: 496; (c) a CDR-H3 comprising the amino acid sequence set forth in SEQ ID NO: 534; (d) a CDR-L1 comprising the amino acid sequence set forth in SEQ ID NO: 559; (e) a CDR-L2 comprising the amino acid sequence set forth SEQ ID NO: 584; and/or (f) a CDR-L3 comprising the amino acid sequence set forth SEQ ID NO: 609. In another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 473; (b) a CDR-H2 comprising the amino acid sequence set forth in SEQ ID NO: 498; (c) a CDR-H3 comprising the amino acid sequence set forth in SEQ ID NO: 536; (d) a CDR-L1 comprising the amino acid sequence set forth in SEQ ID NO: 561; (e) a CDR-L2 comprising the amino acid sequence set forth SEQ ID NO: 586; and/or (f) a CDR-L3 comprising the amino acid sequence set forth SEQ ID NO: 611. In yet another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 474; (b) a CDR-H2 comprising the amino acid sequence set forth in SEQ ID NO: 499; (c) a CDR-H3 comprising the amino acid sequence set forth in SEQ ID NO: 537; (d) a CDR-L1 comprising the amino acid sequence set forth in SEQ ID NO: 562; (e) a CDR-L2 comprising the amino acid sequence set forth SEQ ID NO: 587; and/or (f) a CDR-L3 comprising the amino acid sequence set forth SEQ ID NO: 612. In one embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 632; (b) a CDR-H2 comprising the amino acid sequence set forth in SEQ ID NO: 646; (c) a CDR-H3 comprising the amino acid sequence set forth in SEQ ID NO: 660; (d) a CDR-L1 comprising the amino acid sequence set forth in SEQ ID NO: 674; (e) a CDR-L2 comprising the amino acid sequence set forth SEQ ID NO: 688; and/or (f) a CDR-L3 comprising the amino acid sequence set forth SEQ ID NO: 702.

Likewise, in one embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 640; (b) a CDR-H2 comprising the amino acid sequence set forth in SEQ ID NO: 654; (c) a CDR-H3 comprising the amino acid sequence set forth in SEQ ID NO: 668; (d) a CDR-L1 comprising the amino acid sequence set forth in SEQ ID NO: 682; (e) a CDR-L2 comprising the amino acid sequence set forth SEQ ID NO: 696; and/or (f) a CDR-L3 comprising the amino acid sequence set forth SEQ ID NO: 710. In another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 477; (b) a CDR-H2 comprising the amino acid sequence set forth in SEQ ID NO: 515; (c) a CDR-H3 comprising the amino acid sequence set forth in SEQ ID NO: 540; (d) a CDR-L1 comprising the amino acid sequence set forth in SEQ ID NO: 565; (e) a CDR-L2 comprising the amino acid sequence set forth SEQ ID NO: 590; and/or (f) a CDR-L3 comprising the amino acid sequence set forth SEQ ID NO: 615. In yet another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 475; (b) a CDR-H2 comprising the amino acid sequence set forth in SEQ ID NO: 513; (c) a CDR-H3 comprising the amino acid sequence set forth in SEQ ID NO: 538; (d) a CDR-L1 comprising the amino acid sequence set forth in SEQ ID NO: 563; (e) a CDR-L2 comprising the amino acid sequence set forth SEQ ID NO: 588; and/or (f) a CDR-L3 comprising the amino acid sequence set forth SEQ ID NO: 613. In one embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 476; (b) a CDR-H2 comprising the amino acid sequence set forth in SEQ ID NO: 514; (c) a CDR-H3 comprising the amino acid sequence set forth in SEQ ID NO: 539; (d) a CDR-L1 comprising the amino acid sequence set forth in SEQ ID NO: 564; (e) a CDR-L2 comprising the amino acid sequence set forth SEQ ID NO: 589; and/or (f) a CDR-L3 comprising the amino acid sequence set forth SEQ ID NO: 614. In another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 478; (b) a CDR-H2 comprising the amino acid sequence set forth in SEQ ID NO: 516; (c) a CDR-H3 comprising the amino acid sequence set forth in SEQ ID NO: 541; (d) a CDR-L1 comprising the amino acid sequence set forth in SEQ ID NO: 566; (e) a CDR-L2 comprising the amino acid sequence set forth SEQ ID NO: 591; and/or (f) a CDR-L3 comprising the amino acid sequence set forth SEQ ID NO: 616. In yet another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 479; (b) a CDR-H2 comprising the amino acid sequence set forth in SEQ ID NO: 517; (c) a CDR-H3 comprising the amino acid sequence set forth in SEQ ID NO: 542; (d) a CDR-L1 comprising the amino acid sequence set forth in SEQ ID NO: 567; (e) a CDR-L2 comprising the amino acid sequence set forth SEQ ID NO: 592; and/or (f) a CDR-L3 comprising the amino acid sequence set forth SEQ ID NO: 617. In one embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 633; (b) a CDR-H2 comprising the amino acid sequence set forth in SEQ ID NO: 647; (c) a CDR-H3 comprising the amino acid sequence set forth in SEQ ID NO: 661; (d) a CDR-L1 comprising the amino acid sequence set forth in SEQ ID NO: 675; (e) a CDR-L2 comprising the amino acid sequence set forth SEQ ID NO: 689; and/or (f) a CDR-L3 comprising the amino acid sequence set forth SEQ ID NO: 703.

Additionally, in one embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 641; (b) a CDR-H2 comprising the amino acid sequence set forth in SEQ ID NO: 655; (c) a CDR-H3 comprising the amino acid sequence set forth in SEQ ID NO: 669; (d) a CDR-L1 comprising the amino acid sequence set forth in SEQ ID NO: 683; (e) a CDR-L2 comprising the amino acid sequence set forth SEQ ID NO: 697; and/or (f) a CDR-L3 comprising the amino acid sequence set forth SEQ ID NO: 711. In another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 487; (b) a CDR-H2 comprising the amino acid sequence set forth in SEQ ID NO: 525; (c) a CDR-H3 comprising the amino acid sequence set forth in SEQ ID NO: 550; (d) a CDR-L1 comprising the amino acid sequence set forth in SEQ ID NO: 575; (e) a CDR-L2 comprising the amino acid sequence set forth SEQ ID NO: 600; and/or (f) a CDR-L3 comprising the amino acid sequence set forth SEQ ID NO: 625. In yet another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 485; (b) a CDR-H2 comprising the amino acid sequence set forth in SEQ ID NO: 523; (c) a CDR-H3 comprising the amino acid sequence set forth in SEQ ID NO: 548; (d) a CDR-L1 comprising the amino acid sequence set forth in SEQ ID NO: 573; (e) a CDR-L2 comprising the amino acid sequence set forth SEQ ID NO: 598; and/or (f) a CDR-L3 comprising the amino acid sequence set forth SEQ ID NO: 623. In one embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 486; (b) a CDR-H2 comprising the amino acid sequence set forth in SEQ ID NO: 524; (c) a CDR-H3 comprising the amino acid sequence set forth in SEQ ID NO: 549; (d) a CDR-L1 comprising the amino acid sequence set forth in SEQ ID NO: 574; (e) a CDR-L2 comprising the amino acid sequence set forth SEQ ID NO: 599; and/or (f) a CDR-L3 comprising the amino acid sequence set forth SEQ ID NO: 624. In another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 488; (b) a CDR-H2 comprising the amino acid sequence set forth in SEQ ID NO: 526; (c) a CDR-H3 comprising the amino acid sequence set forth in SEQ ID NO: 551; (d) a CDR-L1 comprising the amino acid sequence set forth in SEQ ID NO: 576; (e) a CDR-L2 comprising the amino acid sequence set forth SEQ ID NO: 601; and/or (f) a CDR-L3 comprising the amino acid sequence set forth SEQ ID NO: 626. In yet another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 489; (b) a CDR-H2 comprising the amino acid sequence set forth in SEQ ID NO: 527; (c) a CDR-H3 comprising the amino acid sequence set forth in SEQ ID NO: 552; (d) a CDR-L1 comprising the amino acid sequence set forth in SEQ ID NO: 577; (e) a CDR-L2 comprising the amino acid sequence set forth SEQ ID NO: 602; and/or (f) a CDR-L3 comprising the amino acid sequence set forth SEQ ID NO: 627. In one embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 634; (b) a CDR-H2 comprising the amino acid sequence set forth in SEQ ID NO: 648; (c) a CDR-H3 comprising the amino acid sequence set forth in SEQ ID NO: 662; (d) a CDR-L1 comprising the amino acid sequence set forth in SEQ ID NO: 676; (e) a CDR-L2 comprising the amino acid sequence set forth SEQ ID NO: 690; and/or (f) a CDR-L3 comprising the amino acid sequence set forth SEQ ID NO: 704.

The anti-CD30L in the pharmaceutical composition provided herein can have various CDR sequences as listed in Tables 10-16. Accordingly, in one embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising any CDR-H1 sequence listed in Table 10; (b) a CDR-H2 comprising any CDR-H2 sequence listed in Table 10; (c) a CDR-H3 comprising any CDR-H3 sequence listed in Table 10; (d) a CDR-L1 comprising any CDR-L1 sequence listed in Table 10; (e) a CDR-L2 comprising any CDR-L2 sequence listed in Table 10; and/or (f) a CDR-L3 comprising any CDR-L3 sequence listed in Table 10.

In another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising any CDR-H1 sequence listed in Table 11; (b) a CDR-H2 comprising any CDR-H2 sequence listed in Table 11; (c) a CDR-H3 comprising any CDR-H3 sequence listed in Table 11; (d) a CDR-L1 comprising any CDR-L1 sequence listed in Table 11; (e) a CDR-L2 comprising any CDR-L2 sequence listed in Table 11; and/or (f) a CDR-L3 comprising any CDR-L3 sequence listed in Table 11.

In a further embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising any CDR-H1 sequence listed in Table 12; (b) a CDR-H2 comprising any CDR-H2 sequence listed in Table 12; (c) a CDR-H3 comprising any CDR-H3 sequence listed in Table 12; (d) a CDR-L1 comprising any CDR-L1 sequence listed in Table 12; (e) a CDR-L2 comprising any CDR-L2 sequence listed in Table 12; and/or (f) a CDR-L3 comprising any CDR-L3 sequence listed in Table 12.

In yet another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising any CDR-H1 sequence listed in Table 13; (b) a CDR-H2 comprising any CDR-H2 sequence listed in Table 13; (c) a CDR-H3 comprising any CDR-H3 sequence listed in Table 13; (d) a CDR-L1 comprising any CDR-L1 sequence listed in Table 13; (e) a CDR-L2 comprising any CDR-L2 sequence listed in Table 13; and/or (f) a CDR-L3 comprising any CDR-L3 sequence listed in Table 13.

In one embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising any CDR-H1 sequence listed in Table 14; (b) a CDR-H2 comprising any CDR-H2 sequence listed in Table 14; (c) a CDR-H3 comprising any CDR-H3 sequence listed in Table 14; (d) a CDR-L1 comprising any CDR-L1 sequence listed in Table 14; (e) a CDR-L2 comprising any CDR-L2 sequence listed in Table 14; and/or (f) a CDR-L3 comprising any CDR-L3 sequence listed in Table 14.

In another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising any CDR-H1 sequence listed in Table 15; (b) a CDR-H2 comprising any CDR-H2 sequence listed in Table 15; (c) a CDR-H3 comprising any CDR-H3 sequence listed in Table 15; (d) a CDR-L1 comprising any CDR-L1 sequence listed in Table 15; (e) a CDR-L2 comprising any CDR-L2 sequence listed in Table 15; and/or (f) a CDR-L3 comprising any CDR-L3 sequence listed in Table 15.

In a further embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising any CDR-H1 sequence listed in Table 16; (b) a CDR-H2 comprising any CDR-H2 sequence listed in Table 16; (c) a CDR-H3 comprising any CDR-H3 sequence listed in Table 16; (d) a CDR-L1 comprising any CDR-L1 sequence listed in Table 16; (e) a CDR-L2 comprising any CDR-L2 sequence listed in Table 16; and/or (f) a CDR-L3 comprising any CDR-L3 sequence listed in Table 16.

In yet another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising any CDR-H1 sequence listed in Tables 10-16; (b) a CDR-H2 comprising any CDR-H2 sequence listed in Tables 10-16; (c) a CDR-H3 comprising any CDR-H3 sequence listed in Tables 10-16; (d) a CDR-L1 comprising any CDR-L1 sequence listed in Tables 10-16; (e) a CDR-L2 comprising any CDR-L2 sequence listed in Tables 10-16; and/or (f) a CDR-L3 comprising any CDR-L3 sequence listed in Tables 10-16.

In some embodiments of the pharmaceutical composition comprising an anti-CD30L antibody or antigen binding fragment thereof provided herein including in Section 2 and this Section 4.5, the antibody or antigen binding fragment thereof comprises the CDR-H1, CDR-H2, CDR-H3, CDR-L1, CDR-L2, and CDR-L3 of the corresponding CDR-H1, CDR-H2, CDR-H3, CDR-L1, CDR-L2, and CDR-L3 of any anti-CD30L of the same clone number listed in Tables 10-16 and 27. In some embodiments of the pharmaceutical composition comprising an anti-CD30L antibody or antigen binding fragment thereof provided herein including in Section 2 and this Section 4.5, the antibody or antigen binding fragment thereof comprises the CDR-H1, CDR-H2, CDR-H3, CDR-L1, CDR-L2, and CDR-L3 of the corresponding CDR-H1, CDR-H2, CDR-H3, CDR-L1, CDR-L2, and CDR-L3 of any anti-CD30L of the same clone number listed in Table 10. In some embodiments of the pharmaceutical composition comprising an anti-CD30L antibody or antigen binding fragment thereof provided herein including in Section 2 and this Section 4.5, the antibody or antigen binding fragment thereof comprises the CDR-H1, CDR-H2, CDR-H3, CDR-L1, CDR-L2, and CDR-L3 of the corresponding CDR-H1, CDR-H2, CDR-H3, CDR-L1, CDR-L2, and CDR-L3 of any anti-CD30L of the same clone number listed in Table 11. In some embodiments of the pharmaceutical composition comprising an anti-CD30L antibody or antigen binding fragment thereof provided herein including in Section 2 and this Section 4.5, the antibody or antigen binding fragment thereof comprises the CDR-H1, CDR-H2, CDR-H3, CDR-L1, CDR-L2, and CDR-L3 of the corresponding CDR-H1, CDR-H2, CDR-H3, CDR-L1, CDR-L2, and CDR-L3 of any anti-CD30L of the same clone number listed in Table 12. In some embodiments of the pharmaceutical composition comprising an anti-CD30L antibody or antigen binding fragment thereof provided herein including in Section 2 and this Section 4.5, the antibody or antigen binding fragment thereof comprises the CDR-H1, CDR-H2, CDR-H3, CDR-L1, CDR-L2, and CDR-L3 of the corresponding CDR-H1, CDR-H2, CDR-H3, CDR-L1, CDR-L2, and CDR-L3 of any anti-CD30L of the same clone number listed in Table 13. In some embodiments of the pharmaceutical composition comprising an anti-CD30L antibody or antigen binding fragment thereof provided herein including in Section 2 and this Section 4.5, the antibody or antigen binding fragment thereof comprises the CDR-H1, CDR-H2, CDR-H3, CDR-L1, CDR-L2, and CDR-L3 of the corresponding CDR-H1, CDR-H2, CDR-H3, CDR-L1, CDR-L2, and CDR-L3 of any anti-CD30L of the same clone number listed in Table 14. In some embodiments of the pharmaceutical composition comprising an anti-CD30L antibody or antigen binding fragment thereof provided herein including in Section 2 and this Section 4.5, the antibody or antigen binding fragment thereof comprises the CDR-H1, CDR-H2, CDR-H3, CDR-L1, CDR-L2, and CDR-L3 of the corresponding CDR-H1, CDR-H2, CDR-H3, CDR-L1, CDR-L2, and CDR-L3 of any anti-CD30L of the same clone number listed in Table 15. In some embodiments of the pharmaceutical composition comprising an anti-CD30L antibody or antigen binding fragment thereof provided herein including in Section 2 and this Section 4.5, the antibody or antigen binding fragment thereof comprises the CDR-H1, CDR-H2, CDR-H3, CDR-L1, CDR-L2, and CDR-L3 of the corresponding CDR-H1, CDR-H2, CDR-H3, CDR-L1, CDR-L2, and CDR-L3 of any anti-CD30L of the same clone number listed in Table 16. In some embodiments of the pharmaceutical composition comprising an anti-CD30L antibody or antigen binding fragment thereof provided herein including in Section 2 and this Section 4.5, the antibody or antigen binding fragment thereof comprises the CDR-H1, CDR-H2, CDR-H3, CDR-L1, CDR-L2, and CDR-L3 of the corresponding CDR-H1, CDR-H2, CDR-H3, CDR-L1, CDR-L2, and CDR-L3 of any anti-CD30L of the same clone number listed in Table 27.

In some embodiments of the pharmaceutical composition comprising an anti-CD30L antibody or antigen binding fragment thereof provided herein including in Section 2 and this Section 4.5, the antibody or antigen binding fragment thereof comprises the VH and VL of the corresponding VH and VL of any anti-CD30L of the same clone number in Tables 10-16 and 27. In some embodiments of the pharmaceutical composition comprising an anti-CD30L antibody or antigen binding fragment thereof provided herein including in Section 2 and this Section 4.5, the antibody or antigen binding fragment thereof comprises the VH and VL of the corresponding VH and VL of any anti-CD30L of the same clone number listed in Table 10. In some embodiments of the pharmaceutical composition comprising an anti-CD30L antibody or antigen binding fragment thereof provided herein including in Section 2 and this Section 4.5, the antibody or antigen binding fragment thereof comprises the VH and VL of the corresponding VH and VL of any anti-CD30L of the same clone number listed in Table 11. In some embodiments of the pharmaceutical composition comprising an anti-CD30L antibody or antigen binding fragment thereof provided herein including in Section 2 and this Section 4.5, the antibody or antigen binding fragment thereof comprises the VH and VL of the corresponding VH and VL of any anti-CD30L of the same clone number listed in Table 12. In some embodiments of the pharmaceutical composition comprising an anti-CD30L antibody or antigen binding fragment thereof provided herein including in Section 2 and this Section 4.5, the antibody or antigen binding fragment thereof comprises the VH and VL of the corresponding VH and VL of any anti-CD30L of the same clone number listed in Table 13. In some embodiments of the pharmaceutical composition comprising an anti-CD30L antibody or antigen binding fragment thereof provided herein including in Section 2 and this Section 4.5, the antibody or antigen binding fragment thereof comprises the VH and VL of the corresponding VH and VL of any anti-CD30L of the same clone number listed in Table 14. In some embodiments of the pharmaceutical composition comprising an anti-CD30L antibody or antigen binding fragment thereof provided herein including in Section 2 and this Section 4.5, the antibody or antigen binding fragment thereof comprises the VH and VL of the corresponding VH and VL of any anti-CD30L of the same clone number listed in Table 15. In some embodiments of the pharmaceutical composition comprising an anti-CD30L antibody or antigen binding fragment thereof provided herein including in Section 2 and this Section 4.5, the antibody or antigen binding fragment thereof comprises the VH and VL of the corresponding VH and VL of any anti-CD30L of the same clone number listed in Table 16. In some embodiments of the pharmaceutical composition comprising an anti-CD30L antibody or antigen binding fragment thereof provided herein including in Section 2 and this Section 4.5, the antibody or antigen binding fragment thereof comprises the VH and VL of the corresponding VH and VL of any anti-CD30L of the same clone number listed in Table 27.

The disclosure also provides that the anti-CD30L in the pharmaceutical composition provided herein can comprise various combinations of immunoglobulin heavy chain variable region (VH) and immunoglobulin light chain variable region (VL). In one embodiment of the pharmaceutical composition comprising an anti-CD30L antibody or antigen binding fragment thereof provided herein including in Section 2 and this Section 4.5, the antibody or antigen binding fragment thereof comprises: (a) an immunoglobulin variable region heavy chain (VH) comprising an amino acid sequence having at least about 90, 95, 97, 98, 99, or 100% sequence identity to any one of SEQ ID NOs: 1, 2, 5, 6, 9, 10, 13, 14, 17, 19, 21, 23, 25, 27, 29, and 31; and/or (b) an immunoglobulin variable region light chain (VL) comprising an amino acid sequence having at least about 90, 95, 97, 98, 99, or 100% sequence identity to any one of SEQ ID NOs: 3, 4, 7, 8, 11, 12, 15, 16, 18, 20, 22, 24, 26, 28, and 30. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence having at least about 90, 95, 97, 98, 99, or 100% sequence identity to SEQ ID NO: 1; and/or (b) a VL comprising an amino acid sequence having at least about 90, 95, 97, 98, 99, or 100% sequence identity to SEQ ID NO: 3. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence having at least about 90, 95, 97, 98, 99, or 100% sequence identity to SEQ ID NO: 2; and/or (b) a VL comprising an amino acid sequence having at least about 90, 95, 97, 98, 99, or 100% sequence identity to SEQ ID NO: 4. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence having at least about 90, 95, 97, 98, 99, or 100% sequence identity to SEQ ID NO: 5; and/or (b) a VL comprising an amino acid sequence having at least about 90, 95, 97, 98, 99, or 100% sequence identity to SEQ ID NO: 7. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence having at least about 90, 95, 97, 98, 99, or 100% sequence identity to SEQ ID NO: 6; and/or (b) a VL comprising an amino acid sequence having at least about 90, 95, 97, 98, 99, or 100% sequence identity to SEQ ID NO: 8. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence having at least about 90, 95, 97, 98, 99, or 100% sequence identity to SEQ ID NO: 9; and/or (b) a VL comprising an amino acid sequence having at least about 90, 95, 97, 98, 99, or 100% sequence identity to SEQ ID NO: 11. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence having at least about 90, 95, 97, 98, 99, or 100% sequence identity to SEQ ID NO: 10; and/or (b) a VL comprising an amino acid sequence having at least about 90, 95, 97, 98, 99, or 100% sequence identity to SEQ ID NO: 12. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence having at least about 90, 95, 97, 98, 99, or 100% sequence identity to SEQ ID NO: 13; and/or (b) a VL comprising an amino acid sequence having at least about 90, 95, 97, 98, 99, or 100% sequence identity to SEQ ID NO: 15. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence having at least about 90, 95, 97, 98, 99, or 100% sequence identity to SEQ ID NO: 14; and/or (b) a VL comprising an amino acid sequence having at least about 90, 95, 97, 98, 99, or 100% sequence identity to SEQ ID NO: 16. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence having at least about 90, 95, 97, 98, 99, or 100% sequence identity to SEQ ID NO: 23; and/or (b) a VL comprising an amino acid sequence having at least about 90, 95, 97, 98, 99, or 100% sequence identity to SEQ ID NO: 24. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence having at least about 90, 95, 97, 98, 99, or 100% sequence identity to SEQ ID NO: 25; and/or (b) a VL comprising an amino acid sequence having at least about 90, 95, 97, 98, 99, or 100% sequence identity to SEQ ID NO: 26. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence having at least about 90, 95, 97, 98, 99, or 100% sequence identity to SEQ ID NO: 27; and/or (b) a VL comprising an amino acid sequence having at least about 90, 95, 97, 98, 99, or 100% sequence identity to SEQ ID NO: 28. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence having at least about 90, 95, 97, 98, 99, or 100% sequence identity to SEQ ID NO: 29; and/or (b) a VL comprising an amino acid sequence having at least about 90, 95, 97, 98, 99, or 100% sequence identity to SEQ ID NO: 30. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence having at least about 90, 95, 97, 98, 99, or 100% sequence identity to SEQ ID NO: 31; and/or (b) a VL comprising an amino acid sequence having at least about 90, 95, 97, 98, 99, or 100% sequence identity to SEQ ID NO: 32.

Additionally, in one embodiment of the pharmaceutical composition comprising an anti-CD30L antibody or antigen binding fragment thereof provided herein including in Section 2 and this Section 4.5, the antibody or antigen binding fragment thereof comprises: (a) an immunoglobulin variable region heavy chain (VH) comprising an amino acid sequence having at least about 80, 81, 82, 83, 84, 85, 86, 87, 88, 89, 90, 91, 92, 93, 94, 95, 96, 97, 98, 99% sequence identity to any one of SEQ ID NOs: 1, 2, 5, 6, 9, 10, 13, 14, 17, 19, 21, 23, 25, 27, 29, and 31; and/or (b) an immunoglobulin variable region light chain (VL) comprising an amino acid sequence having at least about 80, 81, 82, 83, 84, 85, 86, 87, 88, 89, 90, 91, 92, 93, 94, 95, 96, 97, 98, 99% sequence identity to any one of SEQ ID NOs: 3, 4, 7, 8, 11, 12, 15, 16, 18, 20, 22, 24, 26, 28, and 30. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 1; and/or (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 3. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 2; and/or (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 4. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 5; and/or (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 7. In the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 6; and/or (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 8. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 9; and/or (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 11. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 10; and/or (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 12. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 13; and/or (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 15. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 14; and/or (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 16. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 23; and/or (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 24. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 25; and/or (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 26. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 27; and/or (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 28. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 29; and/or (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 30. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 31; and/or (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 32.

Furthermore, in one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence set forth in any one of SEQ ID NOs: 1, 2, 5, 6, 9, 10, 13, 14, 17, 19, 21, 23, 25, 27, 29, and 31; and/or (b) a VL comprising an amino acid sequence set forth in any one of SEQ ID NOs: 3, 4, 7, 8, 11, 12, 15, 16, 18, 20, 22, 24, 26, 28, and 30. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence set forth in SEQ ID NO: 1; and/or (b) a VL comprising an amino acid sequence set forth in SEQ ID NO: 3. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence set forth in SEQ ID NO: 2; and/or (b) a VL comprising an amino acid sequence set forth in SEQ ID NO: 4. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence set forth in SEQ ID NO: 5; and/or (b) a VL comprising an amino acid sequence set forth in SEQ ID NO: 7. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence set forth in SEQ ID NO: 6; and/or (b) a VL comprising an amino acid sequence set forth in SEQ ID NO: 8. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence set forth in SEQ ID NO: 9; and/or (b) a VL comprising an amino acid sequence set forth in SEQ ID NO: 11. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence set forth in SEQ ID NO: 10; and/or (b) a VL comprising an amino acid sequence set forth in SEQ ID NO: 12. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence set forth in SEQ ID NO: 13; and/or (b) a VL comprising an amino acid sequence set forth in SEQ ID NO: 15. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence set forth in SEQ ID NO: 14; and/or (b) a VL comprising an amino acid sequence set forth in SEQ ID NO: 16. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence set forth in SEQ ID NO: 23; and/or (b) a VL comprising an amino acid sequence set forth in SEQ ID NO: 24. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence set forth in SEQ ID NO: 25; and/or (b) a VL comprising an amino acid sequence set forth in SEQ ID NO: 26. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence set forth in SEQ ID NO: 27; and/or (b) a VL comprising an amino acid sequence set forth in SEQ ID NO: 28. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence set forth in SEQ ID NO: 29; and/or (b) a VL comprising an amino acid sequence set forth in SEQ ID NO: 30. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence set forth in SEQ ID NO: 31; and/or (b) a VL comprising an amino acid sequence set forth in SEQ ID NO: 32.

The CDRs of the anti-CD30L antibody or antigen binding fragment in the pharmaceutical composition provided herein are also provided by the CDR definitions as described herein (Kabat, Chothia, AbM, Contact, IMGT, and Aho) for the provided VH and VL sequences. Accordingly, in one embodiment of the pharmaceutical composition comprising an anti-CD30L antibody or antigen binding fragment thereof provided herein including in Section 2 and this Section 4.5, the antibody or antigen binding fragment thereof comprises: (a) the CDR-H1, CDR-H2, and CDR-H3 of the corresponding CDR-H1, CDR-H2, and CDR-H3 of the VH sequence set forth in SEQ ID NO: 1; and/or (b) the CDR-L1, CDR-L2, and CDR-L3 of the corresponding CDR-L1, CDR-L2, and CDR-L3 of the VL sequence set forth in SEQ ID NO: 3, wherein the CDRs are determined by Kabat, Chothia, AbM, Contact, IMGT, or Aho numbering scheme. In one embodiment of the pharmaceutical composition comprising an anti-CD30L antibody or antigen binding fragment thereof provided herein including in Section 2 and this Section 4.5, the antibody or antigen binding fragment thereof comprises: (a) the CDR-H1, CDR-H2, and CDR-H3 of the corresponding CDR-H1, CDR-H2, and CDR-H3 of the VH sequence set forth in SEQ ID NO: 2; and/or (b) the CDR-L1, CDR-L2, and CDR-L3 of the corresponding CDR-L1, CDR-L2, and CDR-L3 of the VL sequence set forth in SEQ ID NO: 4, wherein the CDRs are determined by Kabat, Chothia, AbM, Contact, IMGT, or Aho numbering scheme. In one embodiment of the pharmaceutical composition comprising an anti-CD30L antibody or antigen binding fragment thereof provided herein including in Section 2 and this Section 4.5, the antibody or antigen binding fragment thereof comprises: (a) the CDR-H1, CDR-H2, and CDR-H3 of the corresponding CDR-H1, CDR-H2, and CDR-H3 of the VH sequence set forth in SEQ ID NO: 5; and/or (b) the CDR-L1, CDR-L2, and CDR-L3 of the corresponding CDR-L1, CDR-L2, and CDR-L3 of the VL sequence set forth in SEQ ID NO: 7, wherein the CDRs are determined by Kabat, Chothia, AbM, Contact, IMGT, or Aho numbering scheme. In one embodiment of the pharmaceutical composition comprising an anti-CD30L antibody or antigen binding fragment thereof provided herein including in Section 2 and this Section 4.5, the antibody or antigen binding fragment thereof comprises: (a) the CDR-H1, CDR-H2, and CDR-H3 of the corresponding CDR-H1, CDR-H2, and CDR-H3 of the VH sequence set forth in SEQ ID NO: 6; and/or (b) the CDR-L1, CDR-L2, and CDR-L3 of the corresponding CDR-L1, CDR-L2, and CDR-L3 of the VL sequence set forth in SEQ ID NO: 8, wherein the CDRs are determined by Kabat, Chothia, AbM, Contact, IMGT, or Aho numbering scheme. In one embodiment of the pharmaceutical composition comprising an anti-CD30L antibody or antigen binding fragment thereof provided herein including in Section 2 and this Section 4.5, the antibody or antigen binding fragment thereof comprises: (a) the CDR-H1, CDR-H2, and CDR-H3 of the corresponding CDR-H1, CDR-H2, and CDR-H3 of the VH sequence set forth in SEQ ID NO: 9; and/or (b) the CDR-L1, CDR-L2, and CDR-L3 of the corresponding CDR-L1, CDR-L2, and CDR-L3 of the VL sequence set forth in SEQ ID NO: 11, wherein the CDRs are determined by Kabat, Chothia, AbM, Contact, IMGT, or Aho numbering scheme. In one embodiment of the pharmaceutical composition comprising an anti-CD30L antibody or antigen binding fragment thereof provided herein including in Section 2 and this Section 4.5, the antibody or antigen binding fragment thereof comprises: (a) the CDR-H1, CDR-H2, and CDR-H3 of the corresponding CDR-H1, CDR-H2, and CDR-H3 of the VH sequence set forth in SEQ ID NO: 10; and/or (b) the CDR-L1, CDR-L2, and CDR-L3 of the corresponding CDR-L1, CDR-L2, and CDR-L3 of the VL sequence set forth in SEQ ID NO: 12, wherein the CDRs are determined by Kabat, Chothia, AbM, Contact, IMGT, or Aho numbering scheme. In one embodiment of the pharmaceutical composition comprising an anti-CD30L antibody or antigen binding fragment thereof provided herein including in Section 2 and this Section 4.5, the antibody or antigen binding fragment thereof comprises: (a) the CDR-H1, CDR-H2, and CDR-H3 of the corresponding CDR-H1, CDR-H2, and CDR-H3 of the VH sequence set forth in SEQ ID NO: 13; and/or (b) the CDR-L1, CDR-L2, and CDR-L3 of the corresponding CDR-L1, CDR-L2, and CDR-L3 of the VL sequence set forth in SEQ ID NO: 15, wherein the CDRs are determined by Kabat, Chothia, AbM, Contact, IMGT, or Aho numbering scheme. In one embodiment of the pharmaceutical composition comprising an anti-CD30L antibody or antigen binding fragment thereof provided herein including in Section 2 and this Section 4.5, the antibody or antigen binding fragment thereof comprises: (a) the CDR-H1, CDR-H2, and CDR-H3 of the corresponding CDR-H1, CDR-H2, and CDR-H3 of the VH sequence set forth in SEQ ID NO: 14; and/or (b) the CDR-L1, CDR-L2, and CDR-L3 of the corresponding CDR-L1, CDR-L2, and CDR-L3 of the VL sequence set forth in SEQ ID NO: 16, wherein the CDRs are determined by Kabat, Chothia, AbM, Contact, IMGT, or Aho numbering scheme. In one embodiment of the pharmaceutical composition comprising an anti-CD30L antibody or antigen binding fragment thereof provided herein including in Section 2 and this Section 4.5, the antibody or antigen binding fragment thereof comprises: (a) the CDR-H1, CDR-H2, and CDR-H3 of the corresponding CDR-H1, CDR-H2, and CDR-H3 of the VH sequence set forth in SEQ ID NO: 23; and/or (b) the CDR-L1, CDR-L2, and CDR-L3 of the corresponding CDR-L1, CDR-L2, and CDR-L3 of the VL sequence set forth in SEQ ID NO: 24, wherein the CDRs are determined by Kabat, Chothia, AbM, Contact, IMGT, or Aho numbering scheme. In one embodiment of the pharmaceutical composition comprising an anti-CD30L antibody or antigen binding fragment thereof provided herein including in Section 2 and this Section 4.5, the antibody or antigen binding fragment thereof comprises: (a) the CDR-H1, CDR-H2, and CDR-H3 of the corresponding CDR-H1, CDR-H2, and CDR-H3 of the VH sequence set forth in SEQ ID NO: 25; and/or (b) the CDR-L1, CDR-L2, and CDR-L3 of the corresponding CDR-L1, CDR-L2, and CDR-L3 of the VL sequence set forth in SEQ ID NO: 26, wherein the CDRs are determined by Kabat, Chothia, AbM, Contact, IMGT, or Aho numbering scheme. In one embodiment of the pharmaceutical composition comprising an anti-CD30L antibody or antigen binding fragment thereof provided herein including in Section 2 and this Section 4.5, the antibody or antigen binding fragment thereof comprises: (a) the CDR-H1, CDR-H2, and CDR-H3 of the corresponding CDR-H1, CDR-H2, and CDR-H3 of the VH sequence set forth in SEQ ID NO: 27; and/or (b) the CDR-L1, CDR-L2, and CDR-L3 of the corresponding CDR-L1, CDR-L2, and CDR-L3 of the VL sequence set forth in SEQ ID NO: 28, wherein the CDRs are determined by Kabat, Chothia, AbM, Contact, IMGT, or Aho numbering scheme. In one embodiment of the pharmaceutical composition comprising an anti-CD30L antibody or antigen binding fragment thereof provided herein including in Section 2 and this Section 4.5, the antibody or antigen binding fragment thereof comprises: (a) the CDR-H1, CDR-H2, and CDR-H3 of the corresponding CDR-H1, CDR-H2, and CDR-H3 of the VH sequence set forth in SEQ ID NO: 29; and/or (b) the CDR-L1, CDR-L2, and CDR-L3 of the corresponding CDR-L1, CDR-L2, and CDR-L3 of the VL sequence set forth in SEQ ID NO: 30, wherein the CDRs are determined by Kabat, Chothia, AbM, Contact, IMGT, or Aho numbering scheme. In one embodiment of the pharmaceutical composition comprising an anti-CD30L antibody or antigen binding fragment thereof provided herein including in Section 2 and this Section 4.5, the antibody or antigen binding fragment thereof comprises: (a) the CDR-H1, CDR-H2, and CDR-H3 of the corresponding CDR-H1, CDR-H2, and CDR-H3 of the VH sequence set forth in SEQ ID NO: 31; and/or (b) the CDR-L1, CDR-L2, and CDR-L3 of the corresponding CDR-L1, CDR-L2, and CDR-L3 of the VL sequence set forth in SEQ ID NO: 32, wherein the CDRs are determined by Kabat, Chothia, AbM, Contact, IMGT, or Aho numbering scheme.

As is clear from the descriptions above and below, the anti-CD30L antibodies in the pharmaceutical compositions provided herein can further comprise constant regions. In one embodiment of the pharmaceutical composition comprising an anti-CD30L antibody or antigen binding fragment thereof provided herein including in Section 2 and this Section 4.5, the antibody or antigen binding fragment thereof further comprises an immunoglobulin heavy chain constant region. In one embodiment of the pharmaceutical composition comprising an anti-CD30L antibody or antigen binding fragment thereof provided herein including in Section 2 and this Section 4.5, the antibody or antigen binding fragment thereof further comprises an immunoglobulin light chain constant region. In one embodiment of the pharmaceutical composition comprising an anti-CD30L antibody or antigen binding fragment thereof provided herein including in Section 2 and this Section 4.5, the antibody or antigen binding fragment thereof further comprises an immunoglobulin heavy chain constant region and an immunoglobulin light chain constant region. In one embodiment, the heavy chain constant region is an IgG heavy chain constant region. In one embodiment, the light chain constant region is an IgG light chain constant region. In one embodiment, the light chain constant region is a kappa (κ) chain constant region. In one embodiment, the light chain constant region is a lambda (λ) chain constant region. In one embodiment, the heavy chain constant region is a IgG1 heavy chain constant region. In one embodiment, the heavy chain constant region is a IgG2 heavy chain constant region. In one embodiment, the heavy chain constant region is a IgG3 heavy chain constant region. In one embodiment, the heavy chain constant region is a IgG4 heavy chain constant region. In one embodiment, the constant region comprises an amino acid sequence having about 80, 85, 90, 95, 97, 98, 99, or 100% sequence identity to the amino acid sequence set forth by any one of SEQ ID NOs: 500-512. In one embodiment, the constant region comprises an amino acid sequence having about 80, 81, 82, 83, 84, 85, 86, 87, 88, 89, 90, 91, 92, 93, 94, 95, 96, 97, 98, 99% sequence identity to the amino acid sequence set forth by any one of SEQ ID NOs: 500-512. In one embodiment, the constant region comprises the amino acid sequence set forth by any one of SEQ ID NOs: 500-512. In one embodiment, the constant region comprises the amino acid sequence set forth by any one of SEQ ID NO: 500. In one embodiment, the constant region comprises the amino acid sequence set forth by any one of SEQ ID NO: 501. In one embodiment, the constant region comprises the amino acid sequence set forth by any one of SEQ ID NO: 502. In one embodiment, the constant region comprises the amino acid sequence set forth by any one of SEQ ID NO: 503. In one embodiment, the constant region comprises the amino acid sequence set forth by any one of SEQ ID NO: 504. In one embodiment, the constant region comprises the amino acid sequence set forth by any one of SEQ ID NO: 505. In one embodiment, the constant region comprises the amino acid sequence set forth by any one of SEQ ID NO: 506. In one embodiment, the constant region comprises the amino acid sequence set forth by any one of SEQ ID NO: 507. In one embodiment, the constant region comprises the amino acid sequence set forth by any one of SEQ ID NO: 508. In one embodiment, the constant region comprises the amino acid sequence set forth by any one of SEQ ID NO: 509. In one embodiment, the constant region comprises the amino acid sequence set forth by any one of SEQ ID NO: 510. In one embodiment, the constant region comprises the amino acid sequence set forth by any one of SEQ ID NO: 511. In one embodiment, the constant region comprises the amino acid sequence set forth by any one of SEQ ID NO: 512.

The disclosure further provides pharmaceutical composition of anti-CD30L antibody or antigen binding fragments that cross-react with murine CD30L, functionally block both the murine CD30-CD30L interaction, and/or functionally block the CD30L-mediated CD30 signaling and IL-8 releasing in murine cells. Accordingly, in one embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in any one of SEQ ID NOs: 774-779; (b) a CDR-H2 comprising the amino acid sequence set forth in any one of SEQ ID NOs: 780-785; (c) a CDR-H3 comprising the amino acid sequence set forth in any one of SEQ ID NOs: 786-791; (d) a CDR-L1 comprising the amino acid sequence set forth in any one of SEQ ID NOs: 792-797; (e) a CDR-L2 comprising the amino acid sequence set forth in any one of SEQ ID NOs: 798-803; and/or (f) a CDR-L3 comprising the amino acid sequence set forth in any one of SEQ ID NOs: 804-809. In one embodiment of the pharmaceutical composition comprising an anti-CD30L antibody or antigen binding fragment thereof provided herein including in Section 2 and this Section 4.5, the antibody or antigen binding fragment thereof comprises: (a) the CDR-H1, CDR-H2, and CDR-H3 of the corresponding CDR-H1, CDR-H2, and CDR-H3 of the VH sequence set forth in SEQ ID NO: 770; and/or (b) the CDR-L1, CDR-L2, and CDR-L3 of the corresponding CDR-L1, CDR-L2, and CDR-L3 of the VL sequence set forth in SEQ ID NO: 770, wherein the CDRs are determined by Kabat, Chothia, AbM, Contact, IMGT, or Aho numbering scheme.

Specifically, in one embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 774; (b) a CDR-H2 comprising the amino acid sequence set forth in SEQ ID NO: 780; (c) a CDR-H3 comprising the amino acid sequence set forth in SEQ ID NO: 786; (d) a CDR-L1 comprising the amino acid sequence set forth in SEQ ID NO: 792; (e) a CDR-L2 comprising the amino acid sequence set forth SEQ ID NO: 798; and/or (f) a CDR-L3 comprising the amino acid sequence set forth SEQ ID NO: 804. In another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 775; (b) a CDR-H2 comprising the amino acid sequence set forth in SEQ ID NO: 781; (c) a CDR-H3 comprising the amino acid sequence set forth in SEQ ID NO: 787; (d) a CDR-L1 comprising the amino acid sequence set forth in SEQ ID NO: 793; (e) a CDR-L2 comprising the amino acid sequence set forth SEQ ID NO: 799; and/or (f) a CDR-L3 comprising the amino acid sequence set forth SEQ ID NO: 805. In yet another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 776; (b) a CDR-H2 comprising the amino acid sequence set forth in SEQ ID NO: 782; (c) a CDR-H3 comprising the amino acid sequence set forth in SEQ ID NO: 788; (d) a CDR-L1 comprising the amino acid sequence set forth in SEQ ID NO: 794; (e) a CDR-L2 comprising the amino acid sequence set forth SEQ ID NO: 800; and/or (f) a CDR-L3 comprising the amino acid sequence set forth SEQ ID NO: 806. In one embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 777; (b) a CDR-H2 comprising the amino acid sequence set forth in SEQ ID NO: 783; (c) a CDR-H3 comprising the amino acid sequence set forth in SEQ ID NO: 789; (d) a CDR-L1 comprising the amino acid sequence set forth in SEQ ID NO: 795; (e) a CDR-L2 comprising the amino acid sequence set forth SEQ ID NO: 801; and/or (f) a CDR-L3 comprising the amino acid sequence set forth SEQ ID NO: 807. In another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 778; (b) a CDR-H2 comprising the amino acid sequence set forth in SEQ ID NO: 784; (c) a CDR-H3 comprising the amino acid sequence set forth in SEQ ID NO: 790; (d) a CDR-L1 comprising the amino acid sequence set forth in SEQ ID NO: 796; (e) a CDR-L2 comprising the amino acid sequence set forth SEQ ID NO: 802; and/or (f) a CDR-L3 comprising the amino acid sequence set forth SEQ ID NO: 808. In yet another embodiment of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in SEQ ID NO: 779; (b) a CDR-H2 comprising the amino acid sequence set forth in SEQ ID NO: 785; (c) a CDR-H3 comprising the amino acid sequence set forth in SEQ ID NO: 791; (d) a CDR-L1 comprising the amino acid sequence set forth in SEQ ID NO: 797; (e) a CDR-L2 comprising the amino acid sequence set forth SEQ ID NO: 803; and/or (f) a CDR-L3 comprising the amino acid sequence set forth SEQ ID NO: 809.

Additionally, the disclosure provides that the anti-CD30L antibodies or antigen binding fragments in the pharmaceutical compositions provided herein can comprise the combination of immunoglobulin heavy chain variable region (VH) and immunoglobulin light chain variable region (VL) of the antibody or antigen binding fragments that bind to and block murine CD30L. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 770; and (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 771. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 770; or (b) a VL comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 771. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence set forth in SEQ ID NO: 770; and (b) a VL comprising an amino acid set forth in SEQ ID NO: 771. In one embodiment of the pharmaceutical composition comprising an anti-CD30L antibody or antigen binding fragment thereof provided herein including in Section 2 and this Section 4.5, the antibody or antigen binding fragment thereof comprises: (a) a VH comprising an amino acid sequence set forth in SEQ ID NO: 770; or (b) a VL comprising an amino acid set forth in SEQ ID NO: 771.

Similarly, the disclosure provides that the anti-CD30L antibodies in the pharmaceutical compositions provided herein can comprise the combination of immunoglobulin heavy chain (HC) and immunoglobulin light chain (LC) of the antibodies that bind to and block murine CD30L. In one embodiment of the pharmaceutical composition comprising an anti-CD30L antibody provided herein including in Section 2 and this Section 4.5, the antibody comprises: (a) a HC comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 772; and (b) a LC comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 773. In one embodiment of the pharmaceutical composition comprising an anti-CD30L antibody provided herein including in Section 2 and this Section 4.5, the antibody comprises: (a) a HC comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 772; or (b) a LC comprising an amino acid sequence having at least about 80, 81, 82, 83, 84, 85, 86, 87, 88, 89, 90, 91, 92, 93, 94, 95, 96, 97, 98, 99% sequence identity to SEQ ID NO: 773. In one embodiment of the pharmaceutical composition comprising an anti-CD30L antibody provided herein including in Section 2 and this Section 4.5, the antibody comprises: (a) a HC comprising an amino acid sequence set forth in SEQ ID NO: 772; and (b) a LC comprising an amino acid set forth in SEQ ID NO: 773. In one embodiment of the pharmaceutical composition comprising an anti-CD30L antibody provided herein including in Section 2 and this Section 4.5, the antibody comprises: (a) a HC comprising an amino acid sequence set forth in SEQ ID NO: 772; or (b) a LC comprising an amino acid set forth in SEQ ID NO: 773.

The antibody or antigen binding fragment thereof in the pharmaceutical composition provided herein can have various functional properties as described herein, e.g., in Section 5 (EXAMPLES). Accordingly, in one embodiment of the pharmaceutical composition comprising an anti-CD30L antibody or antigen binding fragment thereof provided herein including in Section 2 and this Section 4.5, the antibody or antigen binding fragment thereof blocks a binding interaction between CD30L and CD30. In one embodiment of the pharmaceutical composition comprising an anti-CD30L antibody or antigen binding fragment thereof provided herein including in Section 2 and this Section 4.5, the antibody or antigen binding fragment thereof inhibits a binding interaction between CD30L and CD30. In one embodiment, the interaction between CD30L and CD30 for assessing the blocking or inhibition is determined in an ELISA assay, a cell binding assay with CD30L expressing cells, a KinExA assay, or a surface plasmon resonance (SPR) assay. In one embodiment, the interaction between CD30L and CD30 for assessing the blocking or inhibition is determined in an ELISA assay. In one embodiment, the interaction between CD30L and CD30 for assessing the blocking or inhibition is determined in a cell binding assay with CD30L expressing cells. In one embodiment, the interaction between CD30L and CD30 for assessing the blocking or inhibition is determined in a KinExA assay. In one embodiment, the interaction between CD30L and CD30 for assessing the blocking or inhibition is determined in an SPR assay.

As shown in Example 8, the anti-CD30L in the pharmaceutical composition provided herein have high binding specificity for CD30L, binding specifically to CD30L among 6,232 targets tested. Accordingly, in one embodiment of the pharmaceutical composition comprising an anti-CD30L antibody or antigen binding fragment thereof provided herein including in Section 2 and this Section 4.5, the antibody or antigen binding fragment thereof specifically binds to CD30L. In some embodiments, the ELISA, SPR, KinExA, and/or cell binding assay with CD30L expressing cells are as described and demonstrated in Section 5 (EXAMPLES) (e.g. FIGS. 4A, 4F, and 4G).

As further demonstrated herein, e.g. in Section 5 (EXAMPLES), the anti-CD30L antibody in the pharmaceutical composition provided herein can block or inhibit CD30L-mediated CD30 signaling in cell-based assays. Such cell assays include cytokine (e.g. IL-8) release dual cell assays. Briefly and using IL-8 release as an example of cytokine release, when co-cultured with CD30L expressing cells (e.g. B16 cells expressing humanCD30L or HEK293 expressing cynoCD30L), K299 CD30+ cells release IL-8 upon ligation with and stimulation by the co-cultured CD30L expressing cells (FIG. 4J). Blocking of the CD30L-CD30 interaction can proportionally reduce the release of IL-8 and thus the amount of IL-8 released can be used as a readout for the effectiveness of antibody mediated blocking of CD30L-CD30 interactions between the co-cultured cells. In some embodiments, provided herein are pharmaceutical compositions comprising antibodies or antigen binding fragments that (i) bind to CD30L and (ii) block or inhibit CD30L-mediated CD30 signaling in cell-based assays described herein.

Accordingly, in one embodiment of the pharmaceutical composition comprising an anti-CD30L antibody or antigen binding fragment thereof provided herein including in Section 2 and this Section 4.5, the antibody or antigen binding fragment thereof inhibits interleukin-8 secretion in a cell-based assay. In one embodiment of the pharmaceutical composition comprising an anti-CD30L antibody or antigen binding fragment thereof provided herein including in Section 2 and this Section 4.5, the antibody or antigen binding fragment thereof inhibits interleukin-6 secretion in a cell-based assay. In one embodiment of the pharmaceutical composition comprising an anti-CD30L antibody or antigen binding fragment thereof provided herein including in Section 2 and this Section 4.5, the antibody or antigen binding fragment thereof inhibits both interleukin-8 and interleukin-6 secretion in a cell-based assay. In one embodiment of the pharmaceutical composition comprising an anti-CD30L antibody or antigen binding fragment thereof provided herein including in Section 2 and this Section 4.5, the antibody or antigen binding fragment thereof blocks interleukin-8 secretion in a cell-based assay. In one embodiment of the pharmaceutical composition comprising an anti-CD30L antibody or antigen binding fragment thereof provided herein including in Section 2 and this Section 4.5, the antibody or antigen binding fragment thereof blocks interleukin-6 secretion in a cell-based assay. In one embodiment of the pharmaceutical composition comprising an anti-CD30L antibody or antigen binding fragment thereof provided herein including in Section 2 and this Section 4.5, the antibody or antigen binding fragment thereof blocks both interleukin-8 and interleukin-6 secretion in a cell-based assay. In one embodiment of the pharmaceutical composition comprising an anti-CD30L antibody or antigen binding fragment thereof provided herein including in Section 2 and this Section 4.5 such as those of this paragraph, the cell-based assay is a dual cell assay with a cell expressing CD30 and a cell expressing CD30L.

In one embodiment, provided herein is a pharmaceutical composition comprising an antibody or antigen binding fragment thereof that inhibits interleukin-8 secretion in a cell-based assay. In one embodiment, provided herein is a pharmaceutical composition comprising an antibody or antigen binding fragment thereof that inhibits interleukin-6 secretion in a cell-based assay. In one embodiment, provided herein is a pharmaceutical composition comprising an antibody or antigen binding fragment thereof that inhibits both interleukin-8 and interleukin-6 secretion in a cell-based assay. In one embodiment, provided herein is a pharmaceutical composition comprising an antibody or antigen binding fragment thereof that blocks interleukin-8 secretion in a cell-based assay. In one embodiment, provided herein is a pharmaceutical composition comprising an antibody or antigen binding fragment thereof that blocks interleukin-6 secretion in a cell-based assay. In one embodiment, provided herein is a pharmaceutical composition comprising an antibody or antigen binding fragment thereof that blocks both interleukin-8 and interleukin-6 secretion in a cell-based assay. In one embodiment of the pharmaceutical composition provided herein, including in Section 2 and this Section 4.5 such as those of this paragraph, the cell-based assay is a dual cell assay with a cell expressing CD30 and a cell expressing CD30L.

Similarly, in one embodiment, provided herein is a pharmaceutical composition comprising an anti-CD30L antibody or antigen binding fragment that reduces IBD induced weight loss. In another embodiment, provided herein is a pharmaceutical composition comprising an anti-CD30L antibody or antigen binding fragment that reduces CD induced weight loss. In yet another embodiment, provided herein is a pharmaceutical composition comprising an anti-CD30L antibody or antigen binding fragment that reduces UC induced weight loss. In one embodiment of the pharmaceutical composition provided herein, including in Section 2 and this Section 4.5, the anti-CD30L antibody or antigen binding fragment thereof in the pharmaceutical composition reduces IBD induced weight loss. In one embodiment of the pharmaceutical composition provided herein, including in Section 2 and this Section 4.5, the anti-CD30L antibody or antigen binding fragment thereof in the pharmaceutical composition reduces CD induced weight loss. In one embodiment of the pharmaceutical composition provided herein, including in Section 2 and this Section 4.5, the anti-CD30L antibody or antigen binding fragment thereof in the pharmaceutical composition reduces UC induced weight loss.

Similarly, in one embodiment, provided herein is a pharmaceutical composition comprising an anti-CD30L antibody or antigen binding fragment that reduces IBD induced injury to intestinal mucosal barrier. In another embodiment, provided herein is a pharmaceutical composition comprising an anti-CD30L antibody or antigen binding fragment that reduces CD induced injury to intestinal mucosal barrier. In yet another embodiment, provided herein is a pharmaceutical composition comprising an anti-CD30L antibody or antigen binding fragment that reduces UC induced injury to intestinal mucosal barrier. In one embodiment of the pharmaceutical composition provided herein, including in Section 2 and this Section 4.5, the anti-CD30L antibody or antigen binding fragment thereof in the pharmaceutical composition reduces IBD induced injury to intestinal mucosal barrier. In one embodiment of the pharmaceutical composition provided herein, including in Section 2 and this Section 4.5, the anti-CD30L antibody or antigen binding fragment thereof in the pharmaceutical composition reduces CD induced injury to intestinal mucosal barrier. In one embodiment of the pharmaceutical composition provided herein, including in Section 2 and this Section 4.5, the anti-CD30L antibody or antigen binding fragment thereof in the pharmaceutical composition reduces UC induced injury to intestinal mucosal barrier.

Similarly, in one embodiment, provided herein is a pharmaceutical composition comprising an anti-CD30L antibody or antigen binding fragment that reduces IBD induced intestinal histopathology sum score. In another embodiment, provided herein is a pharmaceutical composition comprising an anti-CD30L antibody or antigen binding fragment that reduces CD induced intestinal histopathology sum score. In yet another embodiment, provided herein is a pharmaceutical composition comprising an anti-CD30L antibody or antigen binding fragment that reduces UC induced intestinal histopathology sum score. In one embodiment of the pharmaceutical composition provided herein, including in Section 2 and this Section 4.5, the anti-CD30L antibody or antigen binding fragment thereof in the pharmaceutical composition reduces IBD induced intestinal histopathology sum score. In one embodiment of the pharmaceutical composition provided herein, including in Section 2 and this Section 4.5, the anti-CD30L antibody or antigen binding fragment thereof in the pharmaceutical composition reduces CD induced intestinal histopathology sum score. In one embodiment of the pharmaceutical composition provided herein, including in Section 2 and this Section 4.5, the anti-CD30L antibody or antigen binding fragment thereof in the pharmaceutical composition reduces UC induced intestinal histopathology sum score.

Similarly, in one embodiment, provided herein is a pharmaceutical composition comprising an anti-CD30L antibody or antigen binding fragment that reduces IBD induced endoscopy score. In another embodiment, provided herein is a pharmaceutical composition comprising an anti-CD30L antibody or antigen binding fragment that reduces CD induced endoscopy score. In yet another embodiment, provided herein is a pharmaceutical composition comprising an anti-CD30L antibody or antigen binding fragment that reduces UC induced endoscopy score. In one embodiment of the pharmaceutical composition provided herein, including in Section 2 and this Section 4.5, the anti-CD30L antibody or antigen binding fragment thereof in the pharmaceutical composition reduces IBD induced endoscopy score. In one embodiment of the pharmaceutical composition provided herein, including in Section 2 and this Section 4.5, the anti-CD30L antibody or antigen binding fragment thereof in the pharmaceutical composition reduces CD induced endoscopy score. In one embodiment of the pharmaceutical composition provided herein, including in Section 2 and this Section 4.5, the anti-CD30L antibody or antigen binding fragment thereof in the pharmaceutical composition reduces UC induced endoscopy score.

The disclosure demonstrated in Section 5 (EXAMPLES) that the anti-CD30L antibody or antigen binding fragment in the pharmaceutical composition binds to CD30L with strong binding affinity and blocked CD30L with high effectiveness. Accordingly, in one embodiment of the pharmaceutical composition comprising an anti-CD30L antibody or antigen binding fragment thereof provided herein including in Section 2 and this Section 4.5, the antibody or antigen binding fragment thereof binds to CD30L with a dissociation equilibrium constant (K_{D}) of no more than 60, no more than 65, no more than 70, no more than 75, no more than 80, no more than 85, no more than 90, no more than 95, no more than 100, no more than 105, no more than 110, no more than 115, no more than 120, no more than 125, no more than 130, no more than 135, no more than 140, no more than 145, no more than 150, no more than 155, no more than 160, no more than 165, no more than 170, no more than 175, no more than 180, no more than 185, no more than 190, no more than 195, no more than 200, no more than 250, no more than 300, no more than 350, no more than 400, no more than 450, no more than 500, no more than 550, no more than 600, no more than 650, no more than 700, no more than 750, no more than 800, no more than 850, no more than 900, no more than 950, or no more than 1000 pM. In one embodiment of the pharmaceutical composition comprising an anti-CD30L antibody or antigen binding fragment thereof provided herein including in Section 2 and this Section 4.5, the antibody or antigen binding fragment thereof binds to CD30L with a dissociation equilibrium constant (K_{D}) of about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 250, about 300, about 350, about 400, about 450, about 500, about 550, about 600, about 650, about 700, about 750, about 800, about 850, about 900, about 950, or about 1000 pM. In one embodiment of the pharmaceutical composition comprising an anti-CD30L antibody or antigen binding fragment thereof provided herein including in Section 2 and this Section 4.5, the antibody or antigen binding fragment thereof binds to CD30L with a K_{D} of no more than 90.5 pM. In one embodiment of the pharmaceutical composition comprising an anti-CD30L antibody or antigen binding fragment thereof provided herein including in Section 2 and this Section 4.5, the antibody or antigen binding fragment thereof binds to CD30L with a K_{D} of no more than 125 pM. In one embodiment of the pharmaceutical composition comprising an anti-CD30L antibody or antigen binding fragment thereof provided herein including in Section 2 and this Section 4.5, the antibody or antigen binding fragment thereof binds to CD30L with a K_{D} of no more than 65.4 pM. In one embodiment of the pharmaceutical composition comprising an anti-CD30L antibody or antigen binding fragment thereof provided herein including in Section 2 and this Section 4.5, the antibody or antigen binding fragment thereof binds to CD30L with a K_{D} of no more than 122 pM. In one embodiment of the pharmaceutical composition comprising an anti-CD30L antibody or antigen binding fragment thereof provided herein including in Section 2 and this Section 4.5, the antibody or antigen binding fragment thereof binds to CD30L with a K_{D} of no more than 51.3 pM. In one embodiment of the pharmaceutical composition comprising an anti-CD30L antibody or antigen binding fragment thereof provided herein including in Section 2 and this Section 4.5, the antibody or antigen binding fragment thereof binds to CD30L with a K_{D} of no more than 244 pM. In one embodiment of the pharmaceutical composition comprising an anti-CD30L antibody or antigen binding fragment thereof provided herein including in Section 2 and this Section 4.5, the antibody or antigen binding fragment thereof binds to CD30L with a K_{D} of about 90.5 pM. In one embodiment of the pharmaceutical composition comprising an anti-CD30L antibody or antigen binding fragment thereof provided herein including in Section 2 and this Section 4.5, the antibody or antigen binding fragment thereof binds to CD30L with a K_{D} of about 125 pM. In one embodiment of the pharmaceutical composition comprising an anti-CD30L antibody or antigen binding fragment thereof provided herein including in Section 2 and this Section 4.5, the antibody or antigen binding fragment thereof binds to CD30L with a K_{D} of about 64.5 pM. In one embodiment of the pharmaceutical composition comprising an anti-CD30L antibody or antigen binding fragment thereof provided herein including in Section 2 and this Section 4.5, the antibody or antigen binding fragment thereof binds to CD30L with a K_{D} of about 122 pM. In one embodiment of the pharmaceutical composition comprising an anti-CD30L antibody or antigen binding fragment thereof provided herein including in Section 2 and this Section 4.5, the antibody or antigen binding fragment thereof binds to CD30L with a K_{D} of about 51.3 pM. In one embodiment of the pharmaceutical composition comprising an anti-CD30L antibody or antigen binding fragment thereof provided herein including in Section 2 and this Section 4.5, the antibody or antigen binding fragment thereof binds to CD30L with a K_{D} of about 244 pM. In one embodiment of the pharmaceutical composition comprising an anti-CD30L antibody or antigen binding fragment thereof provided herein including in Section 2 and this Section 4.5, the antibody or antigen binding fragment thereof binds to CD30L with a K_{D} of about 65 to about 125 pM. In one embodiment of the pharmaceutical composition comprising an anti-CD30L antibody or antigen binding fragment thereof provided herein including in Section 2 and this Section 4.5, the antibody or antigen binding fragment thereof binds to CD30L with a K_{D} of about 50 to about 140 pM, about 55 to about 135 pM, about 60 to about 130 pM, about 65 to about 125 pM, about 70 to about 120 pM, about 75 to about 115 pM, about 80 to about 110 pM, about 85 to about 105 pM, about 90 to about 100 pM, or about 95 to about 100 pM. In one embodiment of the pharmaceutical composition comprising an anti-CD30L antibody or antigen binding fragment thereof provided herein including in Section 2 and this Section 4.5, the antibody or antigen binding fragment thereof binds to CD30L with a K_{D} of about 51 to about 244 pM. In one embodiment of the pharmaceutical composition comprising an anti-CD30L antibody or antigen binding fragment thereof provided herein including in Section 2 and this Section 4.5, the antibody or antigen binding fragment thereof binds to CD30L with a K_{D} of about 50 to about 250 pM, about 51 to about 244 pM, about 55 to about 240 pM, about 60 to about 235 pM, about 65 to about 230 pM, about 70 to about 225 pM, about 75 to about 220 pM, about 80 to about 215 pM, about 80 to about 210 pM, about 85 to about 205 pM, about 90 to about 200 pM, about 95 to about 195 pM, about 100 to about 190 pM, about 105 to about 185 pM, about 110 to about 180 pM, about 115 to about 175 pM, about 120 to about 170 pM, about 125 to about 165 pM, about 130 to about 160 pM, about 135 to about 155 pM, or about 140 to about 150 pM.

Additionally, in one embodiment of the pharmaceutical composition comprising an anti-CD30L antibody or antigen binding fragment thereof provided herein including in Section 2 and this Section 4.5, the antibody or antigen binding fragment thereof binds to CD30L with an association rate constant (kₒₙ) of at least 0.1×10⁶, at least 0.2×10⁶, at least 0.3×10⁶, at least 0.4×10⁶, at least 0.5×10⁶, at least 0.6×10⁶, at least 0.7×10⁶, at least 0.8×10⁶, at least 0.9×10⁶, at least 1.0×10⁶, at least 1.1×10⁶, at least 1.2×10⁶, at least 1.3×10⁶, at least 1.4×10⁶, at least 1.5×10⁶, at least 1.55×10⁶, at least 1.56×10⁶, at least 1.57×10⁶, at least 1.58×10⁶, at least 1.59×10⁶, at least 1.60×10⁶, at least 1.61×10⁶, at least 1.62×10⁶, at least 1.63×10⁶, at least 1.64×10⁶, at least 1.65×10⁶, at least 1.66×10⁶, at least 1.67×10⁶, at least 1.68×10⁶, at least 1.69×10⁶, at least 1.7×10⁶, at least 1.8×10⁶, at least 1.9×10⁶, at least 2.0×10⁶, at least 2.1×10⁶, at least 2.2×10⁶, at least 2.3×10⁶, at least 2.4×10⁶, at least 2.5×10⁶, at least 2.6×10⁶, at least 2.7×10⁶, at least 2.8×10⁶, at least 2.9×10⁶, at least 3.0×10⁶, or at least 3.1×10⁶M⁻¹ S⁻¹. In one embodiment of the pharmaceutical composition comprising an anti-CD30L antibody or antigen binding fragment thereof provided herein including in Section 2 and this Section 4.5, the antibody or antigen binding fragment thereof binds to CD30L with an association rate constant (kₒₙ) of about 0.1×10⁶, about 0.2×10⁶, about 0.3×10⁶, about 0.4×10⁶, about 0.5×10⁶, about 0.6×10⁶, about 0.7×10⁶, about 0.8×10⁶, about 0.9×10⁶, about 1.0×10⁶, about 1.1×10⁶, about 1.2×10⁶, about 1.3×10⁶, about 1.4×10⁶, about 1.5×10⁶, about 1.55×10⁶, about 1.56×10⁶, about 1.57×10⁶, about 1.58×10⁶, about 1.59×10⁶, about 1.60×10⁶, about 1.61×10⁶M⁻¹ S⁻¹, about 1.62×10⁶, about 1.63×10⁶, about 1.64×10⁶, about 1.65×10⁶, about 1.66×10⁶, about 1.67×10⁶, about 1.68×10⁶, about 1.69×10⁶, about 1.7×10⁶, about 1.8×10⁶, about 1.9×10⁶, about 2.0×10⁶, about 2.1×10⁶, about 2.2×10⁶, about 2.3×10⁶, about 2.4×10⁶, about 2.5×10⁶, about 2.6×10⁶, about 2.7×10⁶, about 2.8×10⁶, about 2.9×10⁶, about 3.0×10⁶, or about 3.1×10⁶M⁻¹ S⁻¹. In one embodiment of the pharmaceutical composition comprising an anti-CD30L antibody or antigen binding fragment thereof provided herein including in Section 2 and this Section 4.5, the antibody or antigen binding fragment thereof binds to CD30L with an association rate constant (kₒₙ) of about 1.54×10⁶ to about 1.55×10⁶, about 1.53×10⁶ to about 1.56×10⁶, about 1.52×10⁶ to about 1.57×10⁶, about 1.53×10⁶ to about 1.58×10⁶, about 1.52×10⁶ to about 1.59×10⁶, about 1.51×10⁶ to about 1.60×10⁶, or about 1.5×10⁶ to about 1.61×10⁶M⁻¹ S⁻¹. In one embodiment of the pharmaceutical composition comprising an anti-CD30L antibody or antigen binding fragment thereof provided herein including in Section 2 and this Section 4.5, the antibody or antigen binding fragment thereof binds to CD30L with an association rate constant (kₒₙ) of about 2.5×10⁶ to about 3.8×10⁶, about 2.4×10⁶ to about 3.9×10⁶, about 2.45×10⁶ to about 3.89×10⁶, about 2.6×10⁶ to about 3.7×10⁶, about 2.7×10⁶ to about 3.6×10⁶, about 2.8×10⁶ to about 3.5×10⁶, about 2.9×10⁶ to about 3.4×10⁶, about 3.0×10⁶ to about 3.3×10⁶, or about 3.1×10⁶ to about 3.2×10⁶, M⁻¹ S⁻¹.

Furthermore, in one embodiment of the pharmaceutical composition comprising an anti-CD30L antibody or antigen binding fragment thereof provided herein including in Section 2 and this Section 4.5, the antibody or antigen binding fragment thereof binds to CD30L with a dissociation rate constant (k_{off}) of no more than 1.4×10⁻⁴, no more than 1.41×10⁻⁴, no more than 1.5×10⁻⁴, no more than 1.6×10⁻⁴, no more than 1.7×10⁻⁴, no more than 1.8×10⁻⁴, no more than 1.9×10⁻⁴, no more than 2.0×10⁻⁴, no more than 2.1×10⁻⁴, no more than 2.2×10⁻⁴, no more than 2.3×10⁻⁴, no more than 2.4×10⁻⁴, no more than 2.5×10⁻⁴, no more than 2.6×10⁻⁴, no more than 2.7×10⁻⁴, no more than 2.8×10⁻⁴, no more than 2.9×10⁻⁴, no more than 3.0×10⁻⁴, no more than 3.1×10⁻⁴, no more than 3.2×10⁻⁴, no more than 3.3×10⁻⁴, no more than 3.4×10⁻⁴, no more than 3.5×10⁻⁴, no more than 3.6×10⁻⁴, no more than 3.7×10⁻⁴, no more than 3.78×10⁻⁴, or no more than 3.8×10⁻⁴ S⁻¹. In one embodiment of the pharmaceutical composition comprising an anti-CD30L antibody or antigen binding fragment thereof provided herein including in Section 2 and this Section 4.5, the antibody or antigen binding fragment thereof binds to CD30L with a dissociation rate constant (k_{off}) of about 1.4×10⁻⁴, about 1.41×10⁻⁴, about 1.5×10⁻⁴, about 1.6×10⁻⁴, about 1.7×10⁻⁴, about 1.8×10⁻⁴, about 1.9×10⁻⁴, about 2.0×10⁻⁴, about 2.1×10⁻⁴, about 2.2×10⁻⁴, about 2.3×10⁻⁴, about 2.4×10⁻⁴, about 2.5×10⁻⁴, about 2.6×10⁻⁴, about 2.7×10⁻⁴, about 2.8×10⁻⁴, about 2.9×10⁻⁴, about 3.0×10⁻⁴, about 3.1×10⁻⁴, about 3.2×10⁻⁴, about 3.3×10⁻⁴, about 3.4×10⁻⁴, about 3.5×10⁻⁴ S⁻¹, about 3.6×10⁻⁴, about 3.7×10⁻⁴, about 3.78×10⁻⁴, or about 3.8×10⁻⁴ S⁻¹.

As described further above, the anti-CD30L in the pharmaceutical composition provided herein binds to specific epitope on CD30L. Such epitope maps differently on to the various CD30L isoforms. Accordingly, the disclosure further provides that the anti-CD30L antibody or antigen binding fragments in the pharmaceutical composition provided herein binds differently to the CD30L isoforms. In one embodiment of the pharmaceutical composition comprising an anti-CD30L antibody or antigen binding fragment thereof provided herein including in Section 2 and this Section 4.5, the antibody or antigen binding fragment thereof binds isoform 1 of CD30L, and wherein the antibody or antigen binding fragment thereof does not bind to isoform 2 of CD30L. In one aspect, provided herein is a pharmaceutical composition comprising an anti-CD30L antibody or antigen binding fragment thereof, wherein the antibody or antigen binding fragment thereof binds isoform 1 of CD30L, and wherein the antibody or antigen binding fragment thereof does not bind to isoform 2 of CD30L. In one embodiment of the pharmaceutical composition comprising an anti-CD30L antibody or antigen binding fragment thereof provided herein including in Section 2 and this Section 4.5 such as this paragraph, the isoform 1 of CD30L comprises the amino acid sequence set forth in SEQ ID NO: 34 and the isoform 2 of CD30L comprises the amino acid sequence set forth in SEQ ID NO: 35. In another embodiment of the pharmaceutical composition comprising an anti-CD30L antibody or antigen binding fragment thereof provided herein including in Section 2 and this Section 4.5 such as this paragraph, the isoform 1 of CD30L consists of the amino acid sequence set forth in SEQ ID NO: 34 and the isoform 2 of CD30L consists of the amino acid sequence set forth in SEQ ID NO: 35.

The disclosure demonstrates that the anti-CD30L in the pharmaceutical composition provided herein binds to human CD30L, cyno CD30L, or both human and cyno CD30L. In some embodiments of the pharmaceutical composition comprising an anti-CD30L antibody or antigen binding fragment thereof provided herein including in Section 2 and this Section 4.5, the antibody or antigen binding fragment thereof binds to human CD30L. In some embodiments of the pharmaceutical composition comprising an anti-CD30L antibody or antigen binding fragment thereof provided herein including in Section 2 and this Section 4.5, the antibody or antigen binding fragment thereof binds to cyno CD30L. In some embodiments of the pharmaceutical composition comprising an anti-CD30L antibody or antigen binding fragment thereof provided herein including in Section 2 and this Section 4.5, the antibody or antigen binding fragment thereof binds to both human and cyno CD30L. In some embodiments of the pharmaceutical composition comprising an anti-CD30L antibody or antigen binding fragment thereof provided herein including in Section 2 and this Section 4.5, the antibody or antigen binding fragment thereof specifically binds to human CD30L. In some embodiments of the pharmaceutical composition comprising an anti-CD30L antibody or antigen binding fragment thereof provided herein including in Section 2 and this Section 4.5, the antibody or antigen binding fragment thereof specifically binds to cyno CD30L. In some embodiments of the pharmaceutical composition comprising an anti-CD30L antibody or antigen binding fragment thereof provided herein including in Section 2 and this Section 4.5, the antibody or antigen binding fragment thereof specifically binds to both human and cyno CD30L.

In certain embodiments of the pharmaceutical composition of anti-CD30L antibody or antigen binding fragment provided herein, including in Section 2 and this Section 4.5, the anti-CD30L antibody or antigen binding fragment thereof in the pharmaceutical composition blocks both the soluble CD30L and the transmembrane CD30L. In one embodiment, the anti-CD30L antibody or antigen binding fragment thereof in the pharmaceutical composition blocks the soluble CD30L. In another embodiment, the anti-CD30L antibody or antigen binding fragment thereof in the pharmaceutical composition blocks the transmembrane CD30L. In some embodiments, the anti-CD30L antibody or antigen binding fragment provided herein, including in Section 2 and this Section (Section 4.1), the anti-CD30L antibody or antigen binding fragment thereof in the pharmaceutical composition binds to both the soluble CD30L and the transmembrane CD30L. In one embodiment, the anti-CD30L antibody or antigen binding fragment thereof in the pharmaceutical composition binds to the soluble CD30L. In another embodiment, the anti-CD30L antibody or antigen binding fragment thereof in the pharmaceutical composition binds to the transmembrane CD30L.

In some embodiments of the pharmaceutical composition comprising an anti-CD30L antibody or antigen binding fragment thereof provided herein including in Section 2 and this Section 4.5, the anti-CD30L antibody or antigen binding fragment thereof: (a) a CDR-H1 comprising the amino acid sequence set forth in any one of SEQ ID NOs: 100-139; (b) a CDR-H2 comprising the amino acid sequence set forth in any one of SEQ ID NOs: 140-179; (c) an immunoglobulin heavy chain CDR3 (CDR-H3) comprising the amino acid sequence set forth in any one of SEQ ID NOs: 180-219; (d) a CDR-L1 comprising the amino acid sequence set forth in any one of SEQ ID NOs: 300-339; (e) a CDR-L2 comprising the amino acid sequence set forth in any one of SEQ ID NOs: 340-379; and/or (f) a CDR-L3 comprising the amino acid sequence set forth in any one of SEQ ID NOs: 380-419.

In some embodiments of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in any one of SEQ ID NOs: 100-109; (b) a CDR-H2 comprising the amino acid sequence set forth in any one of SEQ ID NOs: 140-149; (c) an immunoglobulin heavy chain CDR3 (CDR-H3) comprising the amino acid sequence set forth in any one of SEQ ID NOs: 180-189; (d) a CDR-L1 comprising the amino acid sequence set forth in any one of SEQ ID NOs: 300-309; (e) a CDR-L2 comprising the amino acid sequence set forth in any one of SEQ ID NOs: 340-349; and/or (f) a CDR-L3 comprising the amino acid sequence set forth in any one of SEQ ID NOs: 380-389.

In certain embodiments of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in any one of SEQ ID NOs: 100-104; (b) a CDR-H2 comprising the amino acid sequence set forth in any one of SEQ ID NOs: 140-144; (c) an immunoglobulin heavy chain CDR3 (CDR-H3) comprising the amino acid sequence set forth in any one of SEQ ID NOs: 180-184; (d) a CDR-L1 comprising the amino acid sequence set forth in any one of SEQ ID NOs: 300-304; (e) a CDR-L2 comprising the amino acid sequence set forth in any one of SEQ ID NOs: 340-344; and/or (f) a CDR-L3 comprising the amino acid sequence set forth in any one of SEQ ID NOs: 380-384.

In certain embodiments of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in any one of SEQ ID NOs: 105-109; (b) a CDR-H2 comprising the amino acid sequence set forth in any one of SEQ ID NOs: 145-149; (c) an immunoglobulin heavy chain CDR3 (CDR-H3) comprising the amino acid sequence set forth in any one of SEQ ID NOs: 185-189; (d) a CDR-L1 comprising the amino acid sequence set forth in any one of SEQ ID NOs: 305-309; (e) a CDR-L2 comprising the amino acid sequence set forth in any one of SEQ ID NOs: 345-349; and/or (f) a CDR-L3 comprising the amino acid sequence set forth in any one of SEQ ID NOs: 385-389.

In some embodiments of the pharmaceutical composition comprising an anti-CD30L antibody or antigen binding fragment thereof provided herein including in Section 2 and this Section 4.5, the antibody or antigen binding fragment thereof comprises an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to any one of SEQ ID NOs: 1 and 2; and/or (b) the immunoglobulin variable region light chain comprises an amino acid sequence having at least about 90, 95, 97, 98, 99, or 100% sequence identity to any one of SEQ ID NOs: 3 and 4. In certain embodiments, the immunoglobulin variable region heavy chain comprises an amino acid sequence having about 90% to about 100% sequence identity to any one of SEQ ID NOs: 1 and 2. In certain embodiments, the immunoglobulin variable region heavy chain comprises an amino acid sequence having at least about 90% sequence identity to any one of SEQ ID NOs: 1 and 2. In certain embodiments, the immunoglobulin variable region heavy chain comprises an amino acid sequence having at most about 100% sequence identity to any one of SEQ ID NOs: 1 and 2. In certain embodiments, the immunoglobulin variable region heavy chain comprises an amino acid sequence having about 90% to about 95% sequence identity to any one of SEQ ID NOs: 1 and 2, about 90% to about 96% sequence identity to any one of SEQ ID NOs: 1 and 2, about 90% to about 97% sequence identity to any one of SEQ ID NOs: 1 and 2, about 90% to about 98% sequence identity to any one of SEQ ID NOs: 1 and 2, about 90% to about 99% sequence identity to any one of SEQ ID NOs: 1 and 2, about 90% to about 100% sequence identity to any one of SEQ ID NOs: 1 and 2, about 95% to about 96% sequence identity to any one of SEQ ID NOs: 1 and 2, about 95% to about 97% sequence identity to any one of SEQ ID NOs: 1 and 2, about 95% to about 98% sequence identity to any one of SEQ ID NOs: 1 and 2, about 95% to about 99% sequence identity to any one of SEQ ID NOs: 1 and 2, about 95% to about 100% sequence identity to any one of SEQ ID NOs: 1 and 2, about 96% to about 97% sequence identity to any one of SEQ ID NOs: 1 and 2, about 96% to about 98% sequence identity to any one of SEQ ID NOs: 1 and 2, about 96% to about 99% sequence identity to any one of SEQ ID NOs: 1 and 2, about 96% to about 100% sequence identity to any one of SEQ ID NOs: 1 and 2, about 97% to about 98% sequence identity to any one of SEQ ID NOs: 1 and 2, about 97% to about 99% sequence identity to any one of SEQ ID NOs: 1 and 2, about 97% to about 100% sequence identity to any one of SEQ ID NOs: 1 and 2, about 98% to about 99% sequence identity to any one of SEQ ID NOs: 1 and 2, about 98% to about 100% sequence identity to any one of SEQ ID NOs: 1 and 2, or about 99% to about 100% sequence identity to any one of SEQ ID NOs: 1 and 2. In certain embodiments, the immunoglobulin variable region heavy chain comprises an amino acid sequence having about 90% sequence identity to any one of SEQ ID NOs: 1 and 2, about 95% sequence identity to any one of SEQ ID NOs: 1 and 2, about 97% sequence identity to any one of SEQ ID NOs: 1 and 2, about 98% sequence identity to any one of SEQ ID NOs: 1 and 2, about 99% sequence identity to any one of SEQ ID NOs: 1 and 2, or about 100% sequence identity to any one of SEQ ID NOs: 1 and 2. In certain embodiments, the immunoglobulin variable region heavy chain comprises an amino acid sequence having about 90% to about 100% sequence identity to any one of SEQ ID NOs: 3 and 4. In certain embodiments, the immunoglobulin variable region heavy chain comprises an amino acid sequence having at least about 90% sequence identity to any one of SEQ ID NOs: 3 and 4. In certain embodiments, the immunoglobulin variable region heavy chain comprises an amino acid sequence having at most about 100% sequence identity to any one of SEQ ID NOs: 3 and 4. In certain embodiments, the immunoglobulin variable region heavy chain comprises an amino acid sequence having about 90% to about 95% sequence identity to any one of SEQ ID NOs: 3 and 4, about 90% to about 96% sequence identity to any one of SEQ ID NOs: 3 and 4, about 90% to about 97% sequence identity to any one of SEQ ID NOs: 3 and 4, about 90% to about 98% sequence identity to any one of SEQ ID NOs: 3 and 4, about 90% to about 99% sequence identity to any one of SEQ ID NOs: 3 and 4, about 90% to about 100% sequence identity to any one of SEQ ID NOs: 3 and 4, about 95% to about 96% sequence identity to any one of SEQ ID NOs: 3 and 4, about 95% to about 97% sequence identity to any one of SEQ ID NOs: 3 and 4, about 95% to about 98% sequence identity to any one of SEQ ID NOs: 3 and 4, about 95% to about 99% sequence identity to any one of SEQ ID NOs: 3 and 4, about 95% to about 100% sequence identity to any one of SEQ ID NOs: 3 and 4, about 96% to about 97% sequence identity to any one of SEQ ID NOs: 3 and 4, about 96% to about 98% sequence identity to any one of SEQ ID NOs: 3 and 4, about 96% to about 99% sequence identity to any one of SEQ ID NOs: 3 and 4, about 96% to about 100% sequence identity to any one of SEQ ID NOs: 3 and 4, about 97% to about 98% sequence identity to any one of SEQ ID NOs: 3 and 4, about 97% to about 99% sequence identity to any one of SEQ ID NOs: 3 and 4, about 97% to about 100% sequence identity to any one of SEQ ID NOs: 3 and 4, about 98% to about 99% sequence identity to any one of SEQ ID NOs: 3 and 4, about 98% to about 100% sequence identity to any one of SEQ ID NOs: 3 and 4, or about 99% to about 100% sequence identity to any one of SEQ ID NOs: 3 and 4. In certain embodiments, the immunoglobulin variable region heavy chain comprises an amino acid sequence having about 90% sequence identity to any one of SEQ ID NOs: 3 and 4, about 95% sequence identity to any one of SEQ ID NOs: 3 and 4, about 97% sequence identity to any one of SEQ ID NOs: 3 and 4, about 98% sequence identity to any one of SEQ ID NOs: 3 and 4, about 99% sequence identity to any one of SEQ ID NOs: 3 and 4, or about 100% sequence identity to any one of SEQ ID NOs: 3 and 4.

In some embodiments of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in any one of SEQ ID NOs: 110-119; (b) a CDR-H2 comprising the amino acid sequence set forth in any one of SEQ ID NOs: 150-159; (c) an immunoglobulin heavy chain CDR3 (CDR-H3) comprising the amino acid sequence set forth in any one of SEQ ID NOs: 190-199; (d) a CDR-L1 comprising the amino acid sequence set forth in any one of SEQ ID NOs: 310-319; (e) a CDR-L2 comprising the amino acid sequence set forth in any one of SEQ ID NOs: 350-359; and/or (f) a CDR-L3 comprising the amino acid sequence set forth in any one of SEQ ID NOs: 390-399.

In certain embodiments of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in any one of SEQ ID NOs: 110-114; (b) a CDR-H2 comprising the amino acid sequence set forth in any one of SEQ ID NOs: 150-154; (c) an immunoglobulin heavy chain CDR3 (CDR-H3) comprising the amino acid sequence set forth in any one of SEQ ID NOs: 190-194; (d) a CDR-L1 comprising the amino acid sequence set forth in any one of SEQ ID NOs: 310-314; (e) a CDR-L2 comprising the amino acid sequence set forth in any one of SEQ ID NOs: 350-354; and/or (f) a CDR-L3 comprising the amino acid sequence set forth in any one of SEQ ID NOs: 390-394.

In certain embodiments of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in any one of SEQ ID NOs: 115-119; (b) a CDR-H2 comprising the amino acid sequence set forth in any one of SEQ ID NOs: 155-159; (c) an immunoglobulin heavy chain CDR3 (CDR-H3) comprising the amino acid sequence set forth in any one of SEQ ID NOs: 195-199; (d) a CDR-L1 comprising the amino acid sequence set forth in any one of SEQ ID NOs: 315-319; (e) a CDR-L2 comprising the amino acid sequence set forth in any one of SEQ ID NOs: 355-359; and/or (f) a CDR-L3 comprising the amino acid sequence set forth in any one of SEQ ID NOs: 395-399.

In some embodiments of the pharmaceutical composition comprising an anti-CD30L antibody or antigen binding fragment thereof provided herein including in Section 2 and this Section 4.5, the antibody or antigen binding fragment thereof comprises an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to any one of SEQ ID NOs: 5 and 6; and/or (b) the immunoglobulin variable region light chain comprises an amino acid sequence having at least about 90, 95, 97, 98, 99, or 100% sequence identity to any one of SEQ ID NOs: 7 and 8. In certain embodiments, the immunoglobulin variable region heavy chain comprises an amino acid sequence having about 90% sequence identity to any one of SEQ ID NOs: 5 and 6 to about 100% sequence identity to any one of SEQ ID NOs: 5 and 6. In certain embodiments, the immunoglobulin variable region heavy chain comprises an amino acid sequence having at least about 90% sequence identity to any one of SEQ ID NOs: 5 and 6. In certain embodiments, the immunoglobulin variable region heavy chain comprises an amino acid sequence having at most about 100% sequence identity to any one of SEQ ID NOs: 5 and 6. In certain embodiments, the immunoglobulin variable region heavy chain comprises an amino acid sequence having about 90% sequence identity to any one of SEQ ID NOs: 5 and 6 to about 95% sequence identity to any one of SEQ ID NOs: 5 and 6, about 90% sequence identity to any one of SEQ ID NOs: 5 and 6 to about 97% sequence identity to any one of SEQ ID NOs: 5 and 6, about 90% sequence identity to any one of SEQ ID NOs: 5 and 6 to about 98% sequence identity to any one of SEQ ID NOs: 5 and 6, about 90% sequence identity to any one of SEQ ID NOs: 5 and 6 to about 99% sequence identity to any one of SEQ ID NOs: 5 and 6, about 90% sequence identity to any one of SEQ ID NOs: 5 and 6 to about 100% sequence identity to any one of SEQ ID NOs: 5 and 6, about 95% sequence identity to any one of SEQ ID NOs: 5 and 6 to about 97% sequence identity to any one of SEQ ID NOs: 5 and 6, about 95% sequence identity to any one of SEQ ID NOs: 5 and 6 to about 98% sequence identity to any one of SEQ ID NOs: 5 and 6, about 95% sequence identity to any one of SEQ ID NOs: 5 and 6 to about 99% sequence identity to any one of SEQ ID NOs: 5 and 6, about 95% sequence identity to any one of SEQ ID NOs: 5 and 6 to about 100% sequence identity to any one of SEQ ID NOs: 5 and 6, about 97% sequence identity to any one of SEQ ID NOs: 5 and 6 to about 98% sequence identity to any one of SEQ ID NOs: 5 and 6, about 97% sequence identity to any one of SEQ ID NOs: 5 and 6 to about 99% sequence identity to any one of SEQ ID NOs: 5 and 6, about 97% sequence identity to any one of SEQ ID NOs: 5 and 6 to about 100% sequence identity to any one of SEQ ID NOs: 5 and 6, about 98% sequence identity to any one of SEQ ID NOs: 5 and 6 to about 99% sequence identity to any one of SEQ ID NOs: 5 and 6, about 98% sequence identity to any one of SEQ ID NOs: 5 and 6 to about 100% sequence identity to any one of SEQ ID NOs: 5 and 6, or about 99% sequence identity to any one of SEQ ID NOs: 5 and 6 to about 100% sequence identity to any one of SEQ ID NOs: 5 and 6. In certain embodiments, the immunoglobulin variable region heavy chain comprises an amino acid sequence having about 90% sequence identity to any one of SEQ ID NOs: 5 and 6, about 95% sequence identity to any one of SEQ ID NOs: 5 and 6, about 97% sequence identity to any one of SEQ ID NOs: 5 and 6, about 98% sequence identity to any one of SEQ ID NOs: 5 and 6, about 99% sequence identity to any one of SEQ ID NOs: 5 and 6, or about 100% sequence identity to any one of SEQ ID NOs: 5 and 6. In certain embodiments, the immunoglobulin variable region light chain comprises an amino acid sequence having about 90% sequence identity to any one of SEQ ID NOs: 7 and 8 to about 100% sequence identity to any one of SEQ ID NOs: 7 and 8. In certain embodiments, the immunoglobulin variable region light chain comprises an amino acid sequence having at least about 90% sequence identity to any one of SEQ ID NOs: 7 and 8. In certain embodiments, the immunoglobulin variable region light chain comprises an amino acid sequence having at most about 100% sequence identity to any one of SEQ ID NOs: 7 and 8. In certain embodiments, the immunoglobulin variable region light chain comprises an amino acid sequence having about 90% sequence identity to any one of SEQ ID NOs: 7 and 8 to about 95% sequence identity to any one of SEQ ID NOs: 7 and 8, about 90% sequence identity to any one of SEQ ID NOs: 7 and 8 to about 97% sequence identity to any one of SEQ ID NOs: 7 and 8, about 90% sequence identity to any one of SEQ ID NOs: 7 and 8 to about 98% sequence identity to any one of SEQ ID NOs: 7 and 8, about 90% sequence identity to any one of SEQ ID NOs: 7 and 8 to about 99% sequence identity to any one of SEQ ID NOs: 7 and 8, about 90% sequence identity to any one of SEQ ID NOs: 7 and 8 to about 100% sequence identity to any one of SEQ ID NOs: 7 and 8, about 95% sequence identity to any one of SEQ ID NOs: 7 and 8 to about 97% sequence identity to any one of SEQ ID NOs: 7 and 8, about 95% sequence identity to any one of SEQ ID NOs: 7 and 8 to about 98% sequence identity to any one of SEQ ID NOs: 7 and 8, about 95% sequence identity to any one of SEQ ID NOs: 7 and 8 to about 99% sequence identity to any one of SEQ ID NOs: 7 and 8, about 95% sequence identity to any one of SEQ ID NOs: 7 and 8 to about 100% sequence identity to any one of SEQ ID NOs: 7 and 8, about 97% sequence identity to any one of SEQ ID NOs: 7 and 8 to about 98% sequence identity to any one of SEQ ID NOs: 7 and 8, about 97% sequence identity to any one of SEQ ID NOs: 7 and 8 to about 99% sequence identity to any one of SEQ ID NOs: 7 and 8, about 97% sequence identity to any one of SEQ ID NOs: 7 and 8 to about 100% sequence identity to any one of SEQ ID NOs: 7 and 8, about 98% sequence identity to any one of SEQ ID NOs: 7 and 8 to about 99% sequence identity to any one of SEQ ID NOs: 7 and 8, about 98% sequence identity to any one of SEQ ID NOs: 7 and 8 to about 100% sequence identity to any one of SEQ ID NOs: 7 and 8, or about 99% sequence identity to any one of SEQ ID NOs: 7 and 8 to about 100% sequence identity to any one of SEQ ID NOs: 7 and 8. In certain embodiments, the immunoglobulin variable region light chain comprises an amino acid sequence having about 90% sequence identity to any one of SEQ ID NOs: 7 and 8, about 95% sequence identity to any one of SEQ ID NOs: 7 and 8, about 97% sequence identity to any one of SEQ ID NOs: 7 and 8, about 98% sequence identity to any one of SEQ ID NOs: 7 and 8, about 99% sequence identity to any one of SEQ ID NOs: 7 and 8, or about 100% sequence identity to any one of SEQ ID NOs: 7 and 8.

In some embodiments of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in any one of SEQ ID NOs: 120-129; (b) a CDR-H2 comprising the amino acid sequence set forth in any one of SEQ ID NOs: 160-169; (c) an immunoglobulin heavy chain CDR3 (CDR-H3) comprising the amino acid sequence set forth in any one of SEQ ID NOs: 200-209; (d) a CDR-L1 comprising the amino acid sequence set forth in any one of SEQ ID NOs: 320-329; (e) a CDR-L2 comprising the amino acid sequence set forth in any one of SEQ ID NOs: 360-369; and/or (f) a CDR-L3 comprising the amino acid sequence set forth in any one of SEQ ID NOs: 400-409.

In certain embodiments of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in any one of SEQ ID NOs: 120-124; (b) a CDR-H2 comprising the amino acid sequence set forth in any one of SEQ ID NOs: 160-164; (c) an immunoglobulin heavy chain CDR3 (CDR-H3) comprising the amino acid sequence set forth in any one of SEQ ID NOs: 200-204; (d) a CDR-L1 comprising the amino acid sequence set forth in any one of SEQ ID NOs: 320-324; (e) a CDR-L2 comprising the amino acid sequence set forth in any one of SEQ ID NOs: 360-364; and/or (f) a CDR-L3 comprising the amino acid sequence set forth in any one of SEQ ID NOs: 400-404.

In certain embodiments of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in any one of SEQ ID NOs: 125-129; (b) a CDR-H2 comprising the amino acid sequence set forth in any one of SEQ ID NOs: 165-169; (c) an immunoglobulin heavy chain CDR3 (CDR-H3) comprising the amino acid sequence set forth in any one of SEQ ID NOs: 205-209; (d) a CDR-L1 comprising the amino acid sequence set forth in any one of SEQ ID NOs: 325-329; (e) a CDR-L2 comprising the amino acid sequence set forth in any one of SEQ ID NOs: 365-369; and/or (f) a CDR-L3 comprising the amino acid sequence set forth in any one of SEQ ID NOs: 405-409.

In some embodiments of the pharmaceutical composition comprising an anti-CD30L antibody or antigen binding fragment thereof provided herein including in Section 2 and this Section 4.5, the antibody or antigen binding fragment thereof comprises an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to any one of SEQ ID NOs: 9 and 10; and/or (b) the immunoglobulin variable region light chain comprises an amino acid sequence having at least about 90, 95, 97, 98, 99, or 100% sequence identity to any one of SEQ ID NOs: 11 and 12. In certain embodiments, the immunoglobulin variable region heavy chain comprises an amino acid sequence having about 90% sequence identity to any one of SEQ ID NOs: 9 and 10 to about 100% sequence identity to any one of SEQ ID NOs: 9 and 10. In certain embodiments, the immunoglobulin variable region heavy chain comprises an amino acid sequence having at least about 90% sequence identity to any one of SEQ ID NOs: 9 and 10. In certain embodiments, the immunoglobulin variable region heavy chain comprises an amino acid sequence having at most about 100% sequence identity to any one of SEQ ID NOs: 9 and 10. In certain embodiments, the immunoglobulin variable region heavy chain comprises an amino acid sequence having about 90% sequence identity to any one of SEQ ID NOs: 9 and 10 to about 95% sequence identity to any one of SEQ ID NOs: 9 and 10, about 90% sequence identity to any one of SEQ ID NOs: 9 and 10 to about 97% sequence identity to any one of SEQ ID NOs: 9 and 10, about 90% sequence identity to any one of SEQ ID NOs: 9 and 10 to about 98% sequence identity to any one of SEQ ID NOs: 9 and 10, about 90% sequence identity to any one of SEQ ID NOs: 9 and 10 to about 99% sequence identity to any one of SEQ ID NOs: 9 and 10, about 90% sequence identity to any one of SEQ ID NOs: 9 and 10 to about 100% sequence identity to any one of SEQ ID NOs: 9 and 10, about 95% sequence identity to any one of SEQ ID NOs: 9 and 10 to about 97% sequence identity to any one of SEQ ID NOs: 9 and 10, about 95% sequence identity to any one of SEQ ID NOs: 9 and 10 to about 98% sequence identity to any one of SEQ ID NOs: 9 and 10, about 95% sequence identity to any one of SEQ ID NOs: 9 and 10 to about 99% sequence identity to any one of SEQ ID NOs: 9 and 10, about 95% sequence identity to any one of SEQ ID NOs: 9 and 10 to about 100% sequence identity to any one of SEQ ID NOs: 9 and 10, about 97% sequence identity to any one of SEQ ID NOs: 9 and 10 to about 98% sequence identity to any one of SEQ ID NOs: 9 and 10, about 97% sequence identity to any one of SEQ ID NOs: 9 and 10 to about 99% sequence identity to any one of SEQ ID NOs: 9 and 10, about 97% sequence identity to any one of SEQ ID NOs: 9 and 10 to about 100% sequence identity to any one of SEQ ID NOs: 9 and 10, about 98% sequence identity to any one of SEQ ID NOs: 9 and 10 to about 99% sequence identity to any one of SEQ ID NOs: 9 and 10, about 98% sequence identity to any one of SEQ ID NOs: 9 and 10 to about 100% sequence identity to any one of SEQ ID NOs: 9 and 10, or about 99% sequence identity to any one of SEQ ID NOs: 9 and 10 to about 100% sequence identity to any one of SEQ ID NOs: 9 and 10. In certain embodiments, the immunoglobulin variable region heavy chain comprises an amino acid sequence having about 90% sequence identity to any one of SEQ ID NOs: 9 and 10, about 95% sequence identity to any one of SEQ ID NOs: 9 and 10, about 97% sequence identity to any one of SEQ ID NOs: 9 and 10, about 98% sequence identity to any one of SEQ ID NOs: 9 and 10, about 99% sequence identity to any one of SEQ ID NOs: 9 and 10, or about 100% sequence identity to any one of SEQ ID NOs: 9 and 10. In certain embodiments, the immunoglobulin variable region light chain comprises an amino acid sequence having about 90% sequence identity to any one of SEQ ID NOs: 11 and 12 to about 100% sequence identity to any one of SEQ ID NOs: 11 and 12. In certain embodiments, the immunoglobulin variable region light chain comprises an amino acid sequence having at least about 90% sequence identity to any one of SEQ ID NOs: 11 and 12. In certain embodiments, the immunoglobulin variable region light chain comprises an amino acid sequence having at most about 100% sequence identity to any one of SEQ ID NOs: 11 and 12. In certain embodiments, the immunoglobulin variable region light chain comprises an amino acid sequence having about 90% sequence identity to any one of SEQ ID NOs: 11 and 12 to about 95% sequence identity to any one of SEQ ID NOs: 11 and 12, about 90% sequence identity to any one of SEQ ID NOs: 11 and 12 to about 97% sequence identity to any one of SEQ ID NOs: 11 and 12, about 90% sequence identity to any one of SEQ ID NOs: 11 and 12 to about 98% sequence identity to any one of SEQ ID NOs: 11 and 12, about 90% sequence identity to any one of SEQ ID NOs: 11 and 12 to about 99% sequence identity to any one of SEQ ID NOs: 11 and 12, about 90% sequence identity to any one of SEQ ID NOs: 11 and 12 to about 100% sequence identity to any one of SEQ ID NOs: 11 and 12, about 95% sequence identity to any one of SEQ ID NOs: 11 and 12 to about 97% sequence identity to any one of SEQ ID NOs: 11 and 12, about 95% sequence identity to any one of SEQ ID NOs: 11 and 12 to about 98% sequence identity to any one of SEQ ID NOs: 11 and 12, about 95% sequence identity to any one of SEQ ID NOs: 11 and 12 to about 99% sequence identity to any one of SEQ ID NOs: 11 and 12, about 95% sequence identity to any one of SEQ ID NOs: 11 and 12 to about 100% sequence identity to any one of SEQ ID NOs: 11 and 12, about 97% sequence identity to any one of SEQ ID NOs: 11 and 12 to about 98% sequence identity to any one of SEQ ID NOs: 11 and 12, about 97% sequence identity to any one of SEQ ID NOs: 11 and 12 to about 99% sequence identity to any one of SEQ ID NOs: 11 and 12, about 97% sequence identity to any one of SEQ ID NOs: 11 and 12 to about 100% sequence identity to any one of SEQ ID NOs: 11 and 12, about 98% sequence identity to any one of SEQ ID NOs: 11 and 12 to about 99% sequence identity to any one of SEQ ID NOs: 11 and 12, about 98% sequence identity to any one of SEQ ID NOs: 11 and 12 to about 100% sequence identity to any one of SEQ ID NOs: 11 and 12, or about 99% sequence identity to any one of SEQ ID NOs: 11 and 12 to about 100% sequence identity to any one of SEQ ID NOs: 11 and 12. In certain embodiments, the immunoglobulin variable region light chain comprises an amino acid sequence having about 90% sequence identity to any one of SEQ ID NOs: 11 and 12, about 95% sequence identity to any one of SEQ ID NOs: 11 and 12, about 97% sequence identity to any one of SEQ ID NOs: 11 and 12, about 98% sequence identity to any one of SEQ ID NOs: 11 and 12, about 99% sequence identity to any one of SEQ ID NOs: 11 and 12, or about 100% sequence identity to any one of SEQ ID NOs: 11 and 12.

In some embodiments of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in any one of SEQ ID NOs: 130-139; (b) a CDR-H2 comprising the amino acid sequence set forth in any one of SEQ ID NOs: 170-179; (c) an immunoglobulin heavy chain CDR3 (CDR-H3) comprising the amino acid sequence set forth in any one of SEQ ID NOs: 210-219; (d) a CDR-L1 comprising the amino acid sequence set forth in any one of SEQ ID NOs: 330-339; (e) a CDR-L2 comprising the amino acid sequence set forth in any one of SEQ ID NOs: 370-379; and/or (f) a CDR-L3 comprising the amino acid sequence set forth in any one of SEQ ID NOs: 410-419.

In certain embodiments of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in any one of SEQ ID NOs: 130-134; (b) a CDR-H2 comprising the amino acid sequence set forth in any one of SEQ ID NOs: 170-174; (c) an immunoglobulin heavy chain CDR3 (CDR-H3) comprising the amino acid sequence set forth in any one of SEQ ID NOs: 210-214; (d) a CDR-L1 comprising the amino acid sequence set forth in any one of SEQ ID NOs: 330-334; (e) a CDR-L2 comprising the amino acid sequence set forth in any one of SEQ ID NOs: 370-374; and/or (f) a CDR-L3 comprising the amino acid sequence set forth in any one of SEQ ID NOs: 410-414.

In certain embodiments of the pharmaceutical composition comprising an anti-CD30L antibody or antigen binding fragment thereof provided herein including in Section 2 and this Section 4.5, the antibody or antigen binding fragment thereof comprises: (a) a CDR-H1 comprising the amino acid sequence set forth in any one of SEQ ID NOs: 135-139; (b) a CDR-H2 comprising the amino acid sequence set forth in any one of SEQ ID NOs: 175-179; (c) an immunoglobulin heavy chain CDR3 (CDR-H3) comprising the amino acid sequence set forth in any one of SEQ ID NOs: 215-219; (d) a CDR-L1 comprising the amino acid sequence set forth in any one of SEQ ID NOs: 335-339; (e) a CDR-L2 comprising the amino acid sequence set forth in any one of SEQ ID NOs: 375-379; and/or (f) a CDR-L3 comprising the amino acid sequence set forth in any one of SEQ ID NOs: 415-419.

In some embodiments of the pharmaceutical composition comprising an anti-CD30L antibody or antigen binding fragment thereof provided herein including in Section 2 and this Section 4.5, the antibody or antigen binding fragment thereof comprises an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to any one of SEQ ID NOs: 13 and 14; and/or (b) the immunoglobulin variable region light chain comprises an amino acid sequence having at least about 90, 95, 97, 98, 99, or 100% sequence identity to any one of SEQ ID NOs: 15 and 16. In certain embodiments, the immunoglobulin variable region heavy chain comprises an amino acid sequence having about 90% sequence identity to any one of SEQ ID NOs: 13 and 14 to about 100% sequence identity to any one of SEQ ID NOs: 13 and 14. In certain embodiments, the immunoglobulin variable region heavy chain comprises an amino acid sequence having at least about 90% sequence identity to any one of SEQ ID NOs: 13 and 14. In certain embodiments, the immunoglobulin variable region heavy chain comprises an amino acid sequence having at most about 100% sequence identity to any one of SEQ ID NOs: 13 and 14. In certain embodiments, the immunoglobulin variable region heavy chain comprises an amino acid sequence having about 90% sequence identity to any one of SEQ ID NOs: 13 and 14 to about 95% sequence identity to any one of SEQ ID NOs: 13 and 14, about 90% sequence identity to any one of SEQ ID NOs: 13 and 14 to about 97% sequence identity to any one of SEQ ID NOs: 13 and 14, about 90% sequence identity to any one of SEQ ID NOs: 13 and 14 to about 98% sequence identity to any one of SEQ ID NOs: 13 and 14, about 90% sequence identity to any one of SEQ ID NOs: 13 and 14 to about 99% sequence identity to any one of SEQ ID NOs: 13 and 14, about 90% sequence identity to any one of SEQ ID NOs: 13 and 14 to about 100% sequence identity to any one of SEQ ID NOs: 13 and 14, about 95% sequence identity to any one of SEQ ID NOs: 13 and 14 to about 97% sequence identity to any one of SEQ ID NOs: 13 and 14, about 95% sequence identity to any one of SEQ ID NOs: 13 and 14 to about 98% sequence identity to any one of SEQ ID NOs: 13 and 14, about 95% sequence identity to any one of SEQ ID NOs: 13 and 14 to about 99% sequence identity to any one of SEQ ID NOs: 13 and 14, about 95% sequence identity to any one of SEQ ID NOs: 13 and 14 to about 100% sequence identity to any one of SEQ ID NOs: 13 and 14, about 97% sequence identity to any one of SEQ ID NOs: 13 and 14 to about 98% sequence identity to any one of SEQ ID NOs: 13 and 14, about 97% sequence identity to any one of SEQ ID NOs: 13 and 14 to about 99% sequence identity to any one of SEQ ID NOs: 13 and 14, about 97% sequence identity to any one of SEQ ID NOs: 13 and 14 to about 100% sequence identity to any one of SEQ ID NOs: 13 and 14, about 98% sequence identity to any one of SEQ ID NOs: 13 and 14 to about 99% sequence identity to any one of SEQ ID NOs: 13 and 14, about 98% sequence identity to any one of SEQ ID NOs: 13 and 14 to about 100% sequence identity to any one of SEQ ID NOs: 13 and 14, or about 99% sequence identity to any one of SEQ ID NOs: 13 and 14 to about 100% sequence identity to any one of SEQ ID NOs: 13 and 14. In certain embodiments, the immunoglobulin variable region heavy chain comprises an amino acid sequence having about 90% sequence identity to any one of SEQ ID NOs: 13 and 14, about 95% sequence identity to any one of SEQ ID NOs: 13 and 14, about 97% sequence identity to any one of SEQ ID NOs: 13 and 14, about 98% sequence identity to any one of SEQ ID NOs: 13 and 14, about 99% sequence identity to any one of SEQ ID NOs: 13 and 14, or about 100% sequence identity to any one of SEQ ID NOs: 13 and 14. In certain embodiments, the immunoglobulin variable region light chain comprises an amino acid sequence having about 90% sequence identity to any one of SEQ ID NOs: 15 and 16 to about 100% sequence identity to any one of SEQ ID NOs: 15 and 16. In certain embodiments, the immunoglobulin variable region light chain comprises an amino acid sequence having at least about 90% sequence identity to any one of SEQ ID NOs: 15 and 16. In certain embodiments, the immunoglobulin variable region light chain comprises an amino acid sequence having at most about 100% sequence identity to any one of SEQ ID NOs: 15 and 16. In certain embodiments, the immunoglobulin variable region light chain comprises an amino acid sequence having about 90% sequence identity to any one of SEQ ID NOs: 15 and 16 to about 95% sequence identity to any one of SEQ ID NOs: 15 and 16, about 90% sequence identity to any one of SEQ ID NOs: 15 and 16 to about 97% sequence identity to any one of SEQ ID NOs: 15 and 16, about 90% sequence identity to any one of SEQ ID NOs: 15 and 16 to about 98% sequence identity to any one of SEQ ID NOs: 15 and 16, about 90% sequence identity to any one of SEQ ID NOs: 15 and 16 to about 99% sequence identity to any one of SEQ ID NOs: 15 and 16, about 90% sequence identity to any one of SEQ ID NOs: 15 and 16 to about 100% sequence identity to any one of SEQ ID NOs: 15 and 16, about 95% sequence identity to any one of SEQ ID NOs: 15 and 16 to about 97% sequence identity to any one of SEQ ID NOs: 15 and 16, about 95% sequence identity to any one of SEQ ID NOs: 15 and 16 to about 98% sequence identity to any one of SEQ ID NOs: 15 and 16, about 95% sequence identity to any one of SEQ ID NOs: 15 and 16 to about 99% sequence identity to any one of SEQ ID NOs: 15 and 16, about 95% sequence identity to any one of SEQ ID NOs: 15 and 16 to about 100% sequence identity to any one of SEQ ID NOs: 15 and 16, about 97% sequence identity to any one of SEQ ID NOs: 15 and 16 to about 98% sequence identity to any one of SEQ ID NOs: 15 and 16, about 97% sequence identity to any one of SEQ ID NOs: 15 and 16 to about 99% sequence identity to any one of SEQ ID NOs: 15 and 16, about 97% sequence identity to any one of SEQ ID NOs: 15 and 16 to about 100% sequence identity to any one of SEQ ID NOs: 15 and 16, about 98% sequence identity to any one of SEQ ID NOs: 15 and 16 to about 99% sequence identity to any one of SEQ ID NOs: 15 and 16, about 98% sequence identity to any one of SEQ ID NOs: 15 and 16 to about 100% sequence identity to any one of SEQ ID NOs: 15 and 16, or about 99% sequence identity to any one of SEQ ID NOs: 15 and 16 to about 100% sequence identity to any one of SEQ ID NOs: 15 and 16. In certain embodiments, the immunoglobulin variable region light chain comprises an amino acid sequence having about 90% sequence identity to any one of SEQ ID NOs: 15 and 16, about 95% sequence identity to any one of SEQ ID NOs: 15 and 16, about 97% sequence identity to any one of SEQ ID NOs: 15 and 16, about 98% sequence identity to any one of SEQ ID NOs: 15 and 16, about 99% sequence identity to any one of SEQ ID NOs: 15 and 16, or about 100% sequence identity to any one of SEQ ID NOs: 15 and 16.

In some embodiments of the pharmaceutical composition comprising an anti-CD30L antibody or antigen binding fragment thereof provided herein including in Section 2 and this Section 4.5, the antibody or antigen binding fragment thereof is recombinant antibody or antigen binding fragment thereof.

In certain embodiments of the pharmaceutical composition comprising an anti-CD30L antibody or antigen binding fragment thereof provided herein including in Section 2 and this Section 4.5, the antibody or antigen binding fragment thereof is isolated antibody or antigen binding fragment thereof.

In one additional aspect, provided herein is a pharmaceutical composition comprising an antibody or antigen-binding fragment thereof that binds to an epitope of human CD30L recognized by an anti-CD30L antibody or antigen binding fragment provided herein, including in Section 2 and Section 4.1. Epitope recognized by the anti-CD30L antibodies or antigen binding fragments provided herein can be determined by means described in Sections 4.1 and/or Section 5.

In another aspect, provided herein is a pharmaceutical composition comprising an antibody or antigen-binding fragment thereof that competes for the binding to human CD30L with an anti-CD30L antibody or antigen binding fragment provided herein, including in Section 2 and Section 4.1. Competition of binding to CD30L between the anti-CD30L antibodies (or antigen binding fragments) can be determined by means described in Sections 4.1 and/or Section 5.

In certain embodiments of the pharmaceutical composition comprising an anti-CD30L antibody or antigen binding fragment thereof provided herein including in Section 2 and this Section 4.5, the CD30L is a human CD30L. In some embodiments of the pharmaceutical composition comprising an anti-CD30L antibody or antigen binding fragment thereof provided herein including in Section 2 and this Section 4.5, the CD30L is a cyno CD30L. In certain embodiments of the pharmaceutical composition comprising an anti-CD30L antibody or antigen binding fragment thereof provided herein including in Section 2 and this Section 4.5, the CD30L is both a human CD30L and a cyno CD30L.

### 4.5.1 Concentration of Anti-CD30L Antibody or Antigen Binding Fragment in the Pharmaceutical Composition.

Certain embodiments of the concentrations of the anti-CD30L antibody or antigen binding fragment in the pharmaceutical compositions provided in Section 4.5 are described and provided in in this Section 4.5.1. Accordingly, in certain embodiments, provided herein is a pharmaceutical composition comprising an anti-CD30L antibody or antigen binding fragment thereof provided in Sections 2 and 4.1, at a concentration provided in this Section 4.5.1.

In certain embodiments, provided herein are pharmaceutical compositions comprising an anti-CD30L antibody or antigen binding fragment thereof at a concentration of about or greater than about 150 mg/mL. In certain embodiments, provided herein are pharmaceutical compositions comprising an anti-CD30L antibody or antigen binding fragment thereof at a concentration of additionally provided in the remainder of this paragraph. In some embodiments, the concentration is up to about 300 mg/mL. In some embodiments, the concentration is about or greater than about 155, 160, 165, 170, 175, 180, 185, 190, 195, or 200 mg/mL. In some embodiments, the concentration is about 150 mg/mL to about 300 mg/mL, about 150 mg/mL to about 250 mg/mL, about 150 mg/mL to about 225 mg/mL, about 150 mg/mL to about 220 mg/mL, about 150 mg/mL to about 210 mg/mL, about 150 mg/mL to about 200 mg/mL, about 150 mg/mL to about 190 mg/mL, about 150 mg/mL to about 180 mg/mL, about 160 mg/mL to about 300 mg/mL, about 160 mg/mL to about 250 mg/mL, about 160 mg/mL to about 225 mg/mL, about 160 mg/mL to about 220 mg/mL, about 160 mg/mL to about 210 mg/mL, about 160 mg/mL to about 200 mg/mL, about 160 mg/mL to about 190 mg/mL, about 160 mg/mL to about 180 mg/mL, about 170 mg/mL to about 300 mg/mL, about 170 mg/mL to about 250 mg/mL, about 170 mg/mL to about 225 mg/mL, about 170 mg/mL to about 220 mg/mL, about 170 mg/mL to about 210 mg/mL, about 170 mg/mL to about 200 mg/mL, about 170 mg/mL to about 190 mg/mL, or about 170 mg/mL to about 180 mg/mL. In some embodiments, about 150 mg to about 1,000 mg of the anti-CD30L antibody or antigen binding fragment thereof is present in the composition. For instance, about 150 mg to about 2000 mg, about 150 mg to about 1750 mg, about 150 mg to about 1500 mg, about 150 mg to about 1250 mg, about 150 mg to about 1000 mg, about 150 mg to about 750 mg, about 150 to about 500 mg, about 150 to about 300 mg, about 150 to about 200 mg, or about 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 350, 400, 450, 500, 550, 600, 650, 700, 750, 800, 850, 900, 950, 1000, 1100, 1200, 1300, 1400, 1500, 1600, 1700, 1800, 1900, or 2000 mg of the anti-CD30L antibody or antigen binding fragment thereof can be present in the composition.

Additionally, in some embodiments of the composition provided herein, the composition comprises an anti-CD30L antibody or antigen binding fragment thereof at a concentration greater than about 50 mg/mL. In certain embodiments, provided herein are pharmaceutical compositions comprising an anti-CD30L antibody or antigen binding fragment thereof at a concentration of additionally provided in the remainder of this paragraph. In some embodiments, the pharmaceutical composition comprises an anti-CD30L antibody or antigen binding fragment thereof at a concentration greater than about 55 mg/mL, greater than about 60 mg/mL, greater than about 65 mg/mL, greater than about 70 mg/mL, greater than about 75 mg/mL, greater than about 80 mg/mL, greater than about 85 mg/mL, greater than about 90 mg/mL, greater than about 95 mg/mL, greater than about 100 mg/mL, greater than about 105 mg/mL, greater than about 110 mg/mL, greater than about 115 mg/mL, greater than about 120 mg/mL, greater than about 125 mg/mL, greater than about 130 mg/mL, greater than about 135 mg/mL, greater than about 140 mg/mL, greater than about 145 mg/mL, greater than about 150 mg/mL, greater than about 155 mg/mL, greater than about 160 mg/mL, greater than about 165 mg/mL, greater than about 170 mg/mL, greater than about 175 mg/mL, greater than about 180 mg/mL, greater than about 185 mg/mL, greater than about 190 mg/mL, greater than about 195 mg/mL, greater than about 200 mg/mL, greater than about 205 mg/mL, greater than about 210 mg/mL, greater than about 215 mg/mL, greater than about 220 mg/mL, greater than about 225 mg/mL, greater than about 230 mg/mL, greater than about 235 mg/mL, greater than about 240 mg/mL, greater than about 245 mg/mL, greater than about 250 mg/mL, greater than about 255 mg/mL, greater than about 260 mg/mL, greater than about 265 mg/mL, greater than about 270 mg/mL, greater than about 275 mg/mL, greater than about 280 mg/mL, greater than about 285 mg/mL, greater than about 290 mg/mL, greater than about 295 mg/mL, or greater than about 300 mg/mL. In some embodiments, the pharmaceutical composition comprises an anti-CD30L antibody or antigen binding fragment thereof at a concentration at least about 55 mg/mL, at least about 60 mg/mL, at least about 65 mg/mL, at least about 70 mg/mL, at least about 75 mg/mL, at least about 80 mg/mL, at least about 85 mg/mL, at least about 90 mg/mL, at least about 95 mg/mL, at least about 100 mg/mL, at least about 105 mg/mL, at least about 110 mg/mL, at least about 115 mg/mL, at least about 120 mg/mL, at least about 125 mg/mL, at least about 130 mg/mL, at least about 135 mg/mL, at least about 140 mg/mL, at least about 145 mg/mL, at least about 150 mg/mL, at least about 155 mg/mL, at least about 160 mg/mL, at least about 165 mg/mL, at least about 170 mg/mL, at least about 175 mg/mL, at least about 180 mg/mL, at least about 185 mg/mL, at least about 190 mg/mL, at least about 195 mg/mL, at least about 200 mg/mL, at least about 205 mg/mL, at least about 210 mg/mL, at least about 215 mg/mL, at least about 220 mg/mL, at least about 225 mg/mL, at least about 230 mg/mL, at least about 235 mg/mL, at least about 240 mg/mL, at least about 245 mg/mL, at least about 250 mg/mL, at least about 255 mg/mL, at least about 260 mg/mL, at least about 265 mg/mL, at least about 270 mg/mL, at least about 275 mg/mL, at least about 280 mg/mL, at least about 285 mg/mL, at least about 290 mg/mL, at least about 295 mg/mL, or at least about 300 mg/mL. In some embodiments, the pharmaceutical composition comprises an anti-CD30L antibody or antigen binding fragment thereof at a concentration of about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 105 mg/mL, about 110 mg/mL, about 115 mg/mL, about 120 mg/mL, about 125 mg/mL, about 130 mg/mL, about 135 mg/mL, about 140 mg/mL, about 145 mg/mL, about 150 mg/mL, about 155 mg/mL, about 160 mg/mL, about 165 mg/mL, about 170 mg/mL, about 175 mg/mL, about 180 mg/mL, about 185 mg/mL, about 190 mg/mL, about 195 mg/mL, about 200 mg/mL, about 205 mg/mL, about 210 mg/mL, about 215 mg/mL, about 220 mg/mL, about 225 mg/mL, about 230 mg/mL, about 235 mg/mL, about 240 mg/mL, about 245 mg/mL, about 250 mg/mL, about 255 mg/mL, about 260 mg/mL, about 265 mg/mL, about 270 mg/mL, about 275 mg/mL, about 280 mg/mL, about 285 mg/mL, about 290 mg/mL, about 295 mg/mL, or about 300 mg/mL. In some embodiments, the pharmaceutical composition comprises an anti-CD30L antibody or antigen binding fragment thereof at a concentration of about 50 mg/mL to about 300 mg/ml, about 55 mg/mL to about 300 mg/ml, about 60 mg/mL to about 300 mg/ml, about 65 mg/mL to about 300 mg/ml, about 70 mg/mL to about 300 mg/ml, about 75 mg/mL to about 300 mg/ml, about 80 mg/mL to about 300 mg/ml, about 85 mg/mL to about 300 mg/ml, about 90 mg/mL to about 300 mg/ml, about 95 mg/mL to about 300 mg/ml, about 100 mg/mL to about 300 mg/ml, about 105 mg/mL to about 300 mg/ml, about 110 mg/mL to about 300 mg/ml, about 115 mg/mL to about 300 mg/ml, about 120 mg/mL to about 300 mg/ml, about 125 mg/mL to about 300 mg/ml, about 130 mg/mL to about 300 mg/ml, about 135 mg/mL to about 300 mg/ml, about 140 mg/mL to about 300 mg/ml, about 145 mg/mL to about 300 mg/ml, about 150 mg/mL to about 300 mg/ml, about 155 mg/mL to about 300 mg/ml, about 160 mg/mL to about 300 mg/ml, about 165 mg/mL to about 300 mg/ml, about 170 mg/mL to about 300 mg/ml, about 175 mg/mL to about 300 mg/ml, about 180 mg/mL to about 300 mg/ml, about 185 mg/mL to about 300 mg/ml, about 190 mg/mL to about 300 mg/ml, about 195 mg/mL to about 300 mg/ml, about 200 mg/mL to about 300 mg/ml, about 205 mg/mL to about 300 mg/ml, about 210 mg/mL to about 300 mg/ml, about 215 mg/mL to about 300 mg/ml, about 220 mg/mL to about 300 mg/ml, about 225 mg/mL to about 300 mg/ml, about 230 mg/mL to about 300 mg/ml, about 235 mg/mL to about 300 mg/ml, about 240 mg/mL to about 300 mg/ml, about 245 mg/mL to about 300 mg/ml, about 250 mg/mL to about 300 mg/ml, about 255 mg/mL to about 300 mg/ml, about 260 mg/mL to about 300 mg/ml, about 265 mg/mL to about 300 mg/ml, about 270 mg/mL to about 300 mg/ml, about 275 mg/mL to about 300 mg/ml, about 280 mg/mL to about 300 mg/ml, about 285 mg/mL to about 300 mg/ml, about 290 mg/mL to about 300 mg/ml, or about 295 mg/ml to about 300 mg/ml. In certain embodiments, the pharmaceutical composition comprises an anti-CD30L antibody or antigen binding fragment thereof at a concentration of about 50 mg/mL to about 290 mg/ml, about 55 mg/mL to about 290 mg/ml, about 60 mg/mL to about 290 mg/ml, about 65 mg/mL to about 290 mg/ml, about 70 mg/mL to about 290 mg/ml, about 75 mg/mL to about 290 mg/ml, about 80 mg/mL to about 290 mg/ml, about 85 mg/mL to about 290 mg/ml, about 90 mg/mL to about 290 mg/ml, about 95 mg/mL to about 290 mg/ml, about 100 mg/mL to about 290 mg/ml, about 105 mg/mL to about 290 mg/ml, about 110 mg/mL to about 290 mg/ml, about 115 mg/mL to about 290 mg/ml, about 120 mg/mL to about 290 mg/ml, about 125 mg/mL to about 290 mg/ml, about 130 mg/mL to about 290 mg/ml, about 135 mg/mL to about 290 mg/ml, about 140 mg/mL to about 290 mg/ml, about 145 mg/mL to about 290 mg/ml, about 150 mg/mL to about 290 mg/ml, about 155 mg/mL to about 290 mg/ml, about 160 mg/mL to about 290 mg/ml, about 165 mg/mL to about 290 mg/ml, about 170 mg/mL to about 290 mg/ml, about 175 mg/mL to about 290 mg/ml, about 180 mg/mL to about 290 mg/ml, about 185 mg/mL to about 290 mg/ml, about 190 mg/mL to about 290 mg/ml, about 195 mg/mL to about 290 mg/ml, about 200 mg/mL to about 290 mg/ml, about 205 mg/mL to about 290 mg/ml, about 210 mg/mL to about 290 mg/ml, about 215 mg/mL to about 290 mg/ml, about 220 mg/mL to about 290 mg/ml, about 225 mg/mL to about 290 mg/ml, about 230 mg/mL to about 290 mg/ml, about 235 mg/mL to about 290 mg/ml, about 240 mg/mL to about 290 mg/ml, about 245 mg/mL to about 290 mg/ml, about 250 mg/mL to about 290 mg/ml, about 255 mg/mL to about 290 mg/ml, about 260 mg/mL to about 290 mg/ml, about 265 mg/mL to about 290 mg/ml, about 270 mg/mL to about 290 mg/ml, about 275 mg/mL to about 290 mg/ml, about 280 mg/mL to about 290 mg/ml, or about 285 mg/mL to about 290 mg/ml. In some embodiments, the pharmaceutical composition comprises an anti-CD30L antibody or antigen binding fragment thereof at a concentration of about 50 mg/mL to about 280 mg/ml, about 55 mg/mL to about 280 mg/ml, about 60 mg/mL to about 280 mg/ml, about 65 mg/mL to about 280 mg/ml, about 70 mg/mL to about 280 mg/ml, about 75 mg/mL to about 280 mg/ml, about 80 mg/mL to about 280 mg/ml, about 85 mg/mL to about 280 mg/ml, about 90 mg/mL to about 280 mg/ml, about 95 mg/mL to about 280 mg/ml, about 100 mg/mL to about 280 mg/ml, about 105 mg/mL to about 280 mg/ml, about 110 mg/mL to about 280 mg/ml, about 115 mg/mL to about 280 mg/ml, about 120 mg/mL to about 280 mg/ml, about 125 mg/mL to about 280 mg/ml, about 130 mg/mL to about 280 mg/ml, about 135 mg/mL to about 280 mg/ml, about 140 mg/mL to about 280 mg/ml, about 145 mg/mL to about 280 mg/ml, about 150 mg/mL to about 280 mg/ml, about 155 mg/mL to about 280 mg/ml, about 160 mg/mL to about 280 mg/ml, about 165 mg/mL to about 280 mg/ml, about 170 mg/mL to about 280 mg/ml, about 175 mg/mL to about 280 mg/ml, about 180 mg/mL to about 280 mg/ml, about 185 mg/mL to about 280 mg/ml, about 190 mg/mL to about 280 mg/ml, about 195 mg/mL to about 280 mg/ml, about 200 mg/mL to about 280 mg/ml, about 205 mg/mL to about 280 mg/ml, about 210 mg/mL to about 280 mg/ml, about 215 mg/mL to about 280 mg/ml, about 220 mg/mL to about 280 mg/ml, about 225 mg/mL to about 280 mg/ml, about 230 mg/mL to about 280 mg/ml, about 235 mg/mL to about 280 mg/ml, about 240 mg/mL to about 280 mg/ml, about 245 mg/mL to about 280 mg/ml, about 250 mg/mL to about 280 mg/ml, about 255 mg/mL to about 280 mg/ml, about 260 mg/mL to about 280 mg/ml, about 265 mg/mL to about 280 mg/ml, about 270 mg/mL to about 280 mg/ml, or about 275 mg/mL to about 280 mg/ml. In certain embodiments, the pharmaceutical composition comprises an anti-CD30L antibody or antigen binding fragment thereof at a concentration of about 50 mg/mL to about 275 mg/ml, about 55 mg/mL to about 275 mg/ml, about 60 mg/mL to about 275 mg/ml, about 65 mg/mL to about 275 mg/ml, about 70 mg/mL to about 275 mg/ml, about 75 mg/mL to about 275 mg/ml, about 80 mg/mL to about 275 mg/ml, about 85 mg/mL to about 275 mg/ml, about 90 mg/mL to about 275 mg/ml, about 95 mg/mL to about 275 mg/ml, about 100 mg/mL to about 275 mg/ml, about 105 mg/mL to about 275 mg/ml, about 110 mg/mL to about 275 mg/ml, about 115 mg/mL to about 275 mg/ml, about 120 mg/mL to about 275 mg/ml, about 125 mg/mL to about 275 mg/ml, about 130 mg/mL to about 275 mg/ml, about 135 mg/mL to about 275 mg/ml, about 140 mg/mL to about 275 mg/ml, about 145 mg/mL to about 275 mg/ml, about 150 mg/mL to about 275 mg/ml, about 155 mg/mL to about 275 mg/ml, about 160 mg/mL to about 275 mg/ml, about 165 mg/mL to about 275 mg/ml, about 170 mg/mL to about 275 mg/ml, about 175 mg/mL to about 275 mg/ml, about 180 mg/mL to about 275 mg/ml, about 185 mg/mL to about 275 mg/ml, about 190 mg/mL to about 275 mg/ml, about 195 mg/mL to about 275 mg/ml, about 200 mg/mL to about 275 mg/ml, about 205 mg/mL to about 275 mg/ml, about 210 mg/mL to about 275 mg/ml, about 215 mg/mL to about 275 mg/ml, about 220 mg/mL to about 275 mg/ml, about 225 mg/mL to about 275 mg/ml, about 230 mg/mL to about 275 mg/ml, about 235 mg/mL to about 275 mg/ml, about 240 mg/mL to about 275 mg/ml, about 245 mg/mL to about 275 mg/ml, about 250 mg/mL to about 275 mg/ml, about 255 mg/mL to about 275 mg/ml, about 260 mg/mL to about 275 mg/ml, about 265 mg/mL to about 275 mg/ml, or about 270 mg/mL to about 275 mg/ml. In some embodiments, the pharmaceutical composition comprises an anti-CD30L antibody or antigen binding fragment thereof at a concentration of about 50 mg/mL to about 270 mg/ml, about 55 mg/mL to about 270 mg/ml, about 60 mg/mL to about 270 mg/ml, about 65 mg/mL to about 270 mg/ml, about 70 mg/mL to about 270 mg/ml, about 75 mg/mL to about 270 mg/ml, about 80 mg/mL to about 270 mg/ml, about 85 mg/mL to about 270 mg/ml, about 90 mg/mL to about 270 mg/ml, about 95 mg/mL to about 270 mg/ml, about 100 mg/mL to about 270 mg/ml, about 105 mg/mL to about 270 mg/ml, about 110 mg/mL to about 270 mg/ml, about 115 mg/mL to about 270 mg/ml, about 120 mg/mL to about 270 mg/ml, about 125 mg/mL to about 270 mg/ml, about 130 mg/mL to about 270 mg/ml, about 135 mg/mL to about 270 mg/ml, about 140 mg/mL to about 270 mg/ml, about 145 mg/mL to about 270 mg/ml, about 150 mg/mL to about 270 mg/ml, about 155 mg/mL to about 270 mg/ml, about 160 mg/mL to about 270 mg/ml, about 165 mg/mL to about 270 mg/ml, about 170 mg/mL to about 270 mg/ml, about 175 mg/mL to about 270 mg/ml, about 180 mg/mL to about 270 mg/ml, about 185 mg/mL to about 270 mg/ml, about 190 mg/mL to about 270 mg/ml, about 195 mg/mL to about 270 mg/ml, about 200 mg/mL to about 270 mg/ml, about 205 mg/mL to about 270 mg/ml, about 210 mg/mL to about 270 mg/ml, about 215 mg/mL to about 270 mg/ml, about 220 mg/mL to about 270 mg/ml, about 225 mg/mL to about 270 mg/ml, about 230 mg/mL to about 270 mg/ml, about 235 mg/mL to about 270 mg/ml, about 240 mg/mL to about 270 mg/ml, about 245 mg/mL to about 270 mg/ml, about 250 mg/mL to about 270 mg/ml, about 255 mg/mL to about 270 mg/ml, about 260 mg/mL to about 270 mg/ml, or about 265 mg/mL to about 270 mg/ml. In some embodiments, the pharmaceutical composition comprises an anti-CD30L antibody or antigen binding fragment thereof at a concentration of about 50 mg/mL to about 260 mg/ml, about 55 mg/mL to about 260 mg/ml, about 60 mg/mL to about 260 mg/ml, about 65 mg/mL to about 260 mg/ml, about 70 mg/mL to about 260 mg/ml, about 75 mg/mL to about 260 mg/ml, about 80 mg/mL to about 260 mg/ml, about 85 mg/mL to about 260 mg/ml, about 90 mg/mL to about 260 mg/ml, about 95 mg/mL to about 260 mg/ml, about 100 mg/mL to about 260 mg/ml, about 105 mg/mL to about 260 mg/ml, about 110 mg/mL to about 260 mg/ml, about 115 mg/mL to about 260 mg/ml, about 120 mg/mL to about 260 mg/ml, about 125 mg/mL to about 260 mg/ml, about 130 mg/mL to about 260 mg/ml, about 135 mg/mL to about 260 mg/ml, about 140 mg/mL to about 260 mg/ml, about 145 mg/mL to about 260 mg/ml, about 150 mg/mL to about 260 mg/ml, about 155 mg/mL to about 260 mg/ml, about 160 mg/mL to about 260 mg/ml, about 165 mg/mL to about 260 mg/ml, about 170 mg/mL to about 260 mg/ml, about 175 mg/mL to about 260 mg/ml, about 180 mg/mL to about 260 mg/ml, about 185 mg/mL to about 260 mg/ml, about 190 mg/mL to about 260 mg/ml, about 195 mg/mL to about 260 mg/ml, about 200 mg/mL to about 260 mg/ml, about 205 mg/mL to about 260 mg/ml, about 210 mg/mL to about 260 mg/ml, about 215 mg/mL to about 260 mg/ml, about 220 mg/mL to about 260 mg/ml, about 225 mg/mL to about 260 mg/ml, about 230 mg/mL to about 260 mg/ml, about 235 mg/mL to about 260 mg/ml, about 240 mg/mL to about 260 mg/ml, about 245 mg/mL to about 260 mg/ml, about 250 mg/mL to about 260 mg/ml, or about 255 mg/mL to about 260 mg/ml. In some embodiments, the pharmaceutical composition comprises an anti-CD30L antibody or antigen binding fragment thereof at a concentration of about 50 mg/mL to about 250 mg/mL, about 55 mg/mL to about 250 mg/mL, about 60 mg/mL to about 250 mg/mL, about 65 mg/mL to about 250 mg/mL, about 70 mg/mL to about 250 mg/mL, about 75 mg/mL to about 250 mg/mL, about 80 mg/mL to about 250 mg/mL, about 85 mg/mL to about 250 mg/mL, about 90 mg/mL to about 250 mg/mL, about 95 mg/mL to about 250 mg/mL, about 100 mg/mL to about 250 mg/mL, about 105 mg/mL to about 250 mg/mL, about 110 mg/mL to about 250 mg/mL, about 115 mg/mL to about 250 mg/mL, about 120 mg/mL to about 250 mg/mL, about 125 mg/mL to about 250 mg/mL, about 130 mg/mL to about 250 mg/mL, about 135 mg/mL to about 250 mg/mL, about 140 mg/mL to about 250 mg/mL, about 145 mg/mL to about 250 mg/mL, about 150 mg/mL to about 250 mg/mL, about 155 mg/mL to about 250 mg/mL, about 160 mg/mL to about 250 mg/mL, about 165 mg/mL to about 250 mg/mL, about 170 mg/mL to about 250 mg/mL, about 175 mg/mL to about 250 mg/mL, about 180 mg/mL to about 250 mg/mL, about 185 mg/mL to about 250 mg/mL, about 190 mg/mL to about 250 mg/mL, about 195 mg/mL to about 250 mg/mL, about 200 mg/mL to about 250 mg/mL, about 205 mg/mL to about 250 mg/mL, about 210 mg/mL to about 250 mg/mL, about 215 mg/mL to about 250 mg/mL, about 220 mg/mL to about 250 mg/mL, about 225 mg/mL to about 250 mg/mL, about 230 mg/mL to about 250 mg/mL, about 235 mg/mL to about 250 mg/mL, about 240 mg/mL to about 250 mg/mL, about 245 mg/mL to about 250 mg/mL, about 50 mg/mL to about 240 mg/mL, about 55 mg/mL to about 240 mg/mL, about 60 mg/mL to about 240 mg/mL, about 65 mg/mL to about 240 mg/mL, about 70 mg/mL to about 240 mg/mL, about 75 mg/mL to about 240 mg/mL, about 80 mg/mL to about 240 mg/mL, about 85 mg/mL to about 240 mg/mL, about 90 mg/mL to about 240 mg/mL, about 95 mg/mL to about 240 mg/mL, about 100 mg/mL to about 240 mg/mL, about 105 mg/mL to about 240 mg/mL, about 110 mg/mL to about 240 mg/mL, about 115 mg/mL to about 240 mg/mL, about 120 mg/mL to about 240 mg/mL, about 125 mg/mL to about 240 mg/mL, about 130 mg/mL to about 240 mg/mL, about 135 mg/mL to about 240 mg/mL, about 140 mg/mL to about 240 mg/mL, about 145 mg/mL to about 240 mg/mL, about 150 mg/mL to about 240 mg/mL, about 155 mg/mL to about 240 mg/mL, about 160 mg/mL to about 240 mg/mL, about 165 mg/mL to about 240 mg/mL, about 170 mg/mL to about 240 mg/mL, about 175 mg/mL to about 240 mg/mL, about 180 mg/mL to about 240 mg/mL, about 185 mg/mL to about 240 mg/mL, about 190 mg/mL to about 240 mg/mL, about 195 mg/mL to about 240 mg/mL, about 200 mg/mL to about 240 mg/mL, about 205 mg/mL to about 240 mg/mL, about 210 mg/mL to about 240 mg/mL, about 215 mg/mL to about 240 mg/mL, about 220 mg/mL to about 240 mg/mL, about 225 mg/mL to about 240 mg/mL, about 230 mg/mL to about 240 mg/mL, about 235 mg/mL to about 240 mg/mL, about 50 mg/mL to about 230 mg/mL, about 55 mg/mL to about 230 mg/mL, about 60 mg/mL to about 230 mg/mL, about 65 mg/mL to about 230 mg/mL, about 70 mg/mL to about 230 mg/mL, about 75 mg/mL to about 230 mg/mL, about 80 mg/mL to about 230 mg/mL, about 85 mg/mL to about 230 mg/mL, about 90 mg/mL to about 230 mg/mL, about 95 mg/mL to about 230 mg/mL, about 100 mg/mL to about 230 mg/mL, about 105 mg/mL to about 230 mg/mL, about 110 mg/mL to about 230 mg/mL, about 115 mg/mL to about 230 mg/mL, about 120 mg/mL to about 230 mg/mL, about 125 mg/mL to about 230 mg/mL, about 130 mg/mL to about 230 mg/mL, about 135 mg/mL to about 230 mg/mL, about 140 mg/mL to about 230 mg/mL, about 145 mg/mL to about 230 mg/mL, about 150 mg/mL to about 230 mg/mL, about 155 mg/mL to about 230 mg/mL, about 160 mg/mL to about 230 mg/mL, about 165 mg/mL to about 230 mg/mL, about 170 mg/mL to about 230 mg/mL, about 175 mg/mL to about 230 mg/mL, about 180 mg/mL to about 230 mg/mL, about 185 mg/mL to about 230 mg/mL, about 190 mg/mL to about 230 mg/mL, about 195 mg/mL to about 230 mg/mL, about 200 mg/mL to about 230 mg/mL, about 205 mg/mL to about 230 mg/mL, about 210 mg/mL to about 230 mg/mL, about 215 mg/mL to about 230 mg/mL, about 220 mg/mL to about 230 mg/mL, about 225 mg/mL to about 230 mg/mL, about 50 mg/mL to about 220 mg/mL, about 55 mg/mL to about 220 mg/mL, about 60 mg/mL to about 220 mg/mL, about 65 mg/mL to about 220 mg/mL, about 70 mg/mL to about 220 mg/mL, about 75 mg/mL to about 220 mg/mL, about 80 mg/mL to about 220 mg/mL, about 85 mg/mL to about 220 mg/mL, about 90 mg/mL to about 220 mg/mL, about 95 mg/mL to about 220 mg/mL, about 100 mg/mL to about 220 mg/mL, about 105 mg/mL to about 220 mg/mL, about 110 mg/mL to about 220 mg/mL, about 115 mg/mL to about 220 mg/mL, about 120 mg/mL to about 220 mg/mL, about 125 mg/mL to about 220 mg/mL, about 130 mg/mL to about 220 mg/mL, about 135 mg/mL to about 220 mg/mL, about 140 mg/mL to about 220 mg/mL, about 145 mg/mL to about 220 mg/mL, about 150 mg/mL to about 220 mg/mL, about 155 mg/mL to about 220 mg/mL, about 160 mg/mL to about 220 mg/mL, about 165 mg/mL to about 220 mg/mL, about 170 mg/mL to about 220 mg/mL, about 175 mg/mL to about 220 mg/mL, about 180 mg/mL to about 220 mg/mL, about 185 mg/mL to about 220 mg/mL, about 190 mg/mL to about 220 mg/mL, about 195 mg/mL to about 220 mg/mL, about 200 mg/mL to about 220 mg/mL, about 205 mg/mL to about 220 mg/mL, about 210 mg/mL to about 220 mg/mL, about 215 mg/mL to about 220 mg/mL, about 50 mg/mL to about 210 mg/mL, about 55 mg/mL to about 210 mg/mL, about 60 mg/mL to about 210 mg/mL, about 65 mg/mL to about 210 mg/mL, about 70 mg/mL to about 210 mg/mL, about 75 mg/mL to about 210 mg/mL, about 80 mg/mL to about 210 mg/mL, about 85 mg/mL to about 210 mg/mL, about 90 mg/mL to about 210 mg/mL, about 95 mg/mL to about 210 mg/mL, about 100 mg/mL to about 210 mg/mL, about 105 mg/mL to about 210 mg/mL, about 110 mg/mL to about 210 mg/mL, about 115 mg/mL to about 210 mg/mL, about 120 mg/mL to about 210 mg/mL, about 125 mg/mL to about 210 mg/mL, about 130 mg/mL to about 210 mg/mL, about 135 mg/mL to about 210 mg/mL, about 140 mg/mL to about 210 mg/mL, about 145 mg/mL to about 210 mg/mL, about 150 mg/mL to about 210 mg/mL, about 155 mg/mL to about 210 mg/mL, about 160 mg/mL to about 210 mg/mL, about 165 mg/mL to about 210 mg/mL, about 170 mg/mL to about 210 mg/mL, about 175 mg/mL to about 210 mg/mL, about 180 mg/mL to about 210 mg/mL, about 185 mg/mL to about 210 mg/mL, about 190 mg/mL to about 210 mg/mL, about 195 mg/mL to about 210 mg/mL, about 200 mg/mL to about 210 mg/mL, about 205 mg/mL to about 210 mg/mL, about 50 mg/mL to about 200 mg/mL, about 55 mg/mL to about 200 mg/mL, about 60 mg/mL to about 200 mg/mL, about 65 mg/mL to about 200 mg/mL, about 70 mg/mL to about 200 mg/mL, about 75 mg/mL to about 200 mg/mL, about 80 mg/mL to about 200 mg/mL, about 85 mg/mL to about 200 mg/mL, about 90 mg/mL to about 200 mg/mL, about 95 mg/mL to about 200 mg/mL, about 100 mg/mL to about 200 mg/mL, about 105 mg/mL to about 200 mg/mL, about 110 mg/mL to about 200 mg/mL, about 115 mg/mL to about 200 mg/mL, about 120 mg/mL to about 200 mg/mL, about 125 mg/mL to about 200 mg/mL, about 130 mg/mL to about 200 mg/mL, about 135 mg/mL to about 200 mg/mL, about 140 mg/mL to about 200 mg/mL, about 145 mg/mL to about 200 mg/mL, about 150 mg/mL to about 200 mg/mL, about 155 mg/mL to about 200 mg/mL, about 160 mg/mL to about 200 mg/mL, about 165 mg/mL to about 200 mg/mL, about 170 mg/mL to about 200 mg/mL, about 175 mg/mL to about 200 mg/mL, about 180 mg/mL to about 200 mg/mL, about 185 mg/mL to about 200 mg/mL, about 190 mg/mL to about 200 mg/mL, about 195 mg/mL to about 200 mg/mL, about 50 mg/mL to about 190 mg/mL, about 55 mg/mL to about 190 mg/mL, about 60 mg/mL to about 190 mg/mL, about 65 mg/mL to about 190 mg/mL, about 70 mg/mL to about 190 mg/mL, about 75 mg/mL to about 190 mg/mL, about 80 mg/mL to about 190 mg/mL, about 85 mg/mL to about 190 mg/mL, about 90 mg/mL to about 190 mg/mL, about 95 mg/mL to about 190 mg/mL, about 100 mg/mL to about 190 mg/mL, about 105 mg/mL to about 190 mg/mL, about 110 mg/mL to about 190 mg/mL, about 115 mg/mL to about 190 mg/mL, about 120 mg/mL to about 190 mg/mL, about 125 mg/mL to about 190 mg/mL, about 130 mg/mL to about 190 mg/mL, about 135 mg/mL to about 190 mg/mL, about 140 mg/mL to about 190 mg/mL, about 145 mg/mL to about 190 mg/mL, about 150 mg/mL to about 190 mg/mL, about 155 mg/mL to about 190 mg/mL, about 160 mg/mL to about 190 mg/mL, about 165 mg/mL to about 190 mg/mL, about 170 mg/mL to about 190 mg/mL, about 175 mg/mL to about 190 mg/mL, about 180 mg/mL to about 190 mg/mL, about 185 mg/mL to about 190 mg/mL, about 50 mg/mL to about 180 mg/mL, about 55 mg/mL to about 180 mg/mL, about 60 mg/mL to about 180 mg/mL, about 65 mg/mL to about 180 mg/mL, about 70 mg/mL to about 180 mg/mL, about 75 mg/mL to about 180 mg/mL, about 80 mg/mL to about 180 mg/mL, about 85 mg/mL to about 180 mg/mL, about 90 mg/mL to about 180 mg/mL, about 95 mg/mL to about 180 mg/mL, about 100 mg/mL to about 180 mg/mL, about 105 mg/mL to about 180 mg/mL, about 110 mg/mL to about 180 mg/mL, about 115 mg/mL to about 180 mg/mL, about 120 mg/mL to about 180 mg/mL, about 125 mg/mL to about 180 mg/mL, about 130 mg/mL to about 180 mg/mL, about 135 mg/mL to about 180 mg/mL, about 140 mg/mL to about 180 mg/mL, about 145 mg/mL to about 180 mg/mL, about 150 mg/mL to about 180 mg/mL, about 155 mg/mL to about 180 mg/mL, about 160 mg/mL to about 180 mg/mL, about 165 mg/mL to about 180 mg/mL, about 170 mg/mL to about 180 mg/mL, about 175 mg/mL to about 180 mg/mL, about 50 mg/mL to about 170 mg/mL, about 55 mg/mL to about 170 mg/mL, about 60 mg/mL to about 170 mg/mL, about 65 mg/mL to about 170 mg/mL, about 70 mg/mL to about 170 mg/mL, about 75 mg/mL to about 170 mg/mL, about 80 mg/mL to about 170 mg/mL, about 85 mg/mL to about 170 mg/mL, about 90 mg/mL to about 170 mg/mL, about 95 mg/mL to about 170 mg/mL, about 100 mg/mL to about 170 mg/mL, about 105 mg/mL to about 170 mg/mL, about 110 mg/mL to about 170 mg/mL, about 115 mg/mL to about 170 mg/mL, about 120 mg/mL to about 170 mg/mL, about 125 mg/mL to about 170 mg/mL, about 130 mg/mL to about 170 mg/mL, about 135 mg/mL to about 170 mg/mL, about 140 mg/mL to about 170 mg/mL, about 145 mg/mL to about 170 mg/mL, about 150 mg/mL to about 170 mg/mL, about 155 mg/mL to about 170 mg/mL, about 160 mg/mL to about 170 mg/mL, about 165 mg/mL to about 170 mg/mL, about 50 mg/mL to about 160 mg/mL, about 55 mg/mL to about 160 mg/mL, about 60 mg/mL to about 160 mg/mL, about 65 mg/mL to about 160 mg/mL, about 70 mg/mL to about 160 mg/mL, about 75 mg/mL to about 160 mg/mL, about 80 mg/mL to about 160 mg/mL, about 85 mg/mL to about 160 mg/mL, about 90 mg/mL to about 160 mg/mL, about 95 mg/mL to about 160 mg/mL, about 100 mg/mL to about 160 mg/mL, about 105 mg/mL to about 160 mg/mL, about 110 mg/mL to about 160 mg/mL, about 115 mg/mL to about 160 mg/mL, about 120 mg/mL to about 160 mg/mL, about 125 mg/mL to about 160 mg/mL, about 130 mg/mL to about 160 mg/mL, about 135 mg/mL to about 160 mg/mL, about 140 mg/mL to about 160 mg/mL, about 145 mg/mL to about 160 mg/mL, about 150 mg/mL to about 160 mg/mL, about 155 mg/mL to about 160 mg/mL, about 50 mg/mL to about 150 mg/mL, about 55 mg/mL to about 150 mg/mL, about 60 mg/mL to about 150 mg/mL, about 65 mg/mL to about 150 mg/mL, about 70 mg/mL to about 150 mg/mL, about 75 mg/mL to about 150 mg/mL, about 80 mg/mL to about 150 mg/mL, about 85 mg/mL to about 150 mg/mL, about 90 mg/mL to about 150 mg/mL, about 95 mg/mL to about 150 mg/mL, about 100 mg/mL to about 150 mg/mL, about 105 mg/mL to about 150 mg/mL, about 110 mg/mL to about 150 mg/mL, about 115 mg/mL to about 150 mg/mL, about 120 mg/mL to about 150 mg/mL, about 125 mg/mL to about 150 mg/mL, about 130 mg/mL to about 150 mg/mL, about 135 mg/mL to about 150 mg/mL, about 140 mg/mL to about 150 mg/mL, or about 145 mg/mL to about 150 mg/mL. In some embodiments, the pharmaceutical composition comprises an anti-CD30L antibody or antigen binding fragment thereof at a concentration of about 50 mg/mL to about 140 mg/mL, about 55 mg/mL to about 140 mg/mL, about 60 mg/mL to about 140 mg/mL, about 65 mg/mL to about 140 mg/mL, about 70 mg/mL to about 140 mg/mL, about 75 mg/mL to about 140 mg/mL, about 80 mg/mL to about 140 mg/mL, about 85 mg/mL to about 140 mg/mL, about 90 mg/mL to about 140 mg/mL, about 95 mg/mL to about 140 mg/mL, about 100 mg/mL to about 140 mg/mL, about 105 mg/mL to about 140 mg/mL, about 110 mg/mL to about 140 mg/mL, about 115 mg/mL to about 140 mg/mL, about 120 mg/mL to about 140 mg/mL, about 125 mg/mL to about 140 mg/mL, about 130 mg/mL to about 140 mg/mL, or about 135 mg/mL to about 140 mg/mL. In some embodiments, the pharmaceutical composition comprises an anti-CD30L antibody or antigen binding fragment thereof at a concentration of about 50 mg/mL to about 130 mg/mL, about 55 mg/mL to about 130 mg/mL, about 60 mg/mL to about 130 mg/mL, about 65 mg/mL to about 130 mg/mL, about 70 mg/mL to about 130 mg/mL, about 75 mg/mL to about 130 mg/mL, about 80 mg/mL to about 130 mg/mL, about 85 mg/mL to about 130 mg/mL, about 90 mg/mL to about 130 mg/mL, about 95 mg/mL to about 130 mg/mL, about 100 mg/mL to about 130 mg/mL, about 105 mg/mL to about 130 mg/mL, about 110 mg/mL to about 130 mg/mL, about 115 mg/mL to about 130 mg/mL, about 120 mg/mL to about 130 mg/mL, or about 125 mg/mL to about 130 mg/mL. In some embodiments, the pharmaceutical composition comprises an anti-CD30L antibody or antigen binding fragment thereof at a concentration of about 50 mg/mL to about 120 mg/mL, about 55 mg/mL to about 120 mg/mL, about 60 mg/mL to about 120 mg/mL, about 65 mg/mL to about 120 mg/mL, about 70 mg/mL to about 120 mg/mL, about 75 mg/mL to about 120 mg/mL, about 80 mg/mL to about 120 mg/mL, about 85 mg/mL to about 120 mg/mL, about 90 mg/mL to about 120 mg/mL, about 95 mg/mL to about 120 mg/mL, about 100 mg/mL to about 120 mg/mL, about 105 mg/mL to about 120 mg/mL, about 110 mg/mL to about 120 mg/mL, or about 115 mg/mL to about 120 mg/mL. In some embodiments, the pharmaceutical composition comprises an anti-CD30L antibody or antigen binding fragment thereof at a concentration of about 50 mg/mL to about 110 mg/mL, about 55 mg/mL to about 110 mg/mL, about 60 mg/mL to about 110 mg/mL, about 65 mg/mL to about 110 mg/mL, about 70 mg/mL to about 110 mg/mL, about 75 mg/mL to about 110 mg/mL, about 80 mg/mL to about 110 mg/mL, about 85 mg/mL to about 110 mg/mL, about 90 mg/mL to about 110 mg/mL, about 95 mg/mL to about 110 mg/mL, about 100 mg/mL to about 110 mg/mL, or about 105 mg/mL to about 110 mg/mL. In some embodiments, the pharmaceutical composition comprises an anti-CD30L antibody or antigen binding fragment thereof at a concentration of about 50 mg/mL to about 100 mg/mL, about 55 mg/mL to about 100 mg/mL, about 60 mg/mL to about 100 mg/mL, about 65 mg/mL to about 100 mg/mL, about 70 mg/mL to about 100 mg/mL, about 75 mg/mL to about 100 mg/mL, about 80 mg/mL to about 100 mg/mL, about 85 mg/mL to about 100 mg/mL, about 90 mg/mL to about 100 mg/mL, about 95 mg/mL to about 100 mg/mL, about 100 mg/mL to about 100 mg/mL, or about 105 mg/mL to about 100 mg/mL. In some embodiments, the pharmaceutical composition comprises an anti-CD30L antibody or antigen binding fragment thereof at a concentration of about 50 mg/mL to about 90 mg/mL, about 55 mg/mL to about 90 mg/mL, about 60 mg/mL to about 90 mg/mL, about 65 mg/mL to about 90 mg/mL, about 70 mg/mL to about 90 mg/mL, about 75 mg/mL to about 90 mg/mL, about 80 mg/mL to about 90 mg/mL, or about 85 mg/mL to about 90 mg/mL. In some embodiments, the pharmaceutical composition comprises an anti-CD30L antibody or antigen binding fragment thereof at a concentration of about 50 mg/mL to about 80 mg/mL, about 55 mg/mL to about 80 mg/mL, about 60 mg/mL to about 80 mg/mL, about 65 mg/mL to about 80 mg/mL, about 70 mg/mL to about 80 mg/mL, or about 75 mg/mL to about 80 mg/mL. In some embodiments, the pharmaceutical composition comprises an anti-CD30L antibody or antigen binding fragment thereof at a concentration of about 50 mg/mL to about 70 mg/mL, about 55 mg/mL to about 70 mg/mL, about 60 mg/mL to about 70 mg/mL, or about 65 mg/mL to about 70 mg/mL. In some embodiments, the pharmaceutical composition comprises an anti-CD30L antibody or antigen binding fragment thereof at a concentration of about 50 mg/mL to about 55 mg/mL, about 50 mg/mL to about 60 mg/mL, or about 55 mg/mL to about 60 mg/mL.

Additionally, provided herein are pharmaceutical compositions comprising an anti-CD30L antibody or antigen binding fragment thereof at a concentration of described in this paragraph. In some embodiments, the pharmaceutical composition comprises an anti-CD30L antibody or antigen binding fragment thereof at a concentration from about 50 mg/ml to about 275 mg/ml. In some embodiments, the pharmaceutical composition comprises an anti-CD30L antibody or antigen binding fragment thereof at a concentration from about 50 mg/ml to about 250 mg/ml. In some embodiments, the pharmaceutical composition comprises an anti-CD30L antibody or antigen binding fragment thereof at a concentration from about 50 mg/ml to about 225 mg/ml. In some embodiments, the pharmaceutical composition comprises an anti-CD30L antibody or antigen binding fragment thereof at a concentration from about 50 mg/ml to about 200 mg/ml. In some embodiments, the pharmaceutical composition comprises an anti-CD30L antibody or antigen binding fragment thereof at a concentration from about 50 mg/ml to about 175 mg/ml. In some embodiments, the pharmaceutical composition comprises an anti-CD30L antibody or antigen binding fragment thereof at a concentration from about 50 mg/ml to about 150 mg/ml. In some embodiments, the pharmaceutical composition comprises an anti-CD30L antibody or antigen binding fragment thereof at a concentration from about 50 mg/ml to about 125 mg/ml. In some embodiments, the pharmaceutical composition comprises an anti-CD30L antibody or antigen binding fragment thereof at a concentration from about 50 mg/ml to about 100 mg/ml. In some embodiments, the pharmaceutical composition comprises an anti-CD30L antibody or antigen binding fragment thereof at a concentration from about 50 mg/ml to about 75 mg/ml. In some embodiments, the pharmaceutical composition comprises an anti-CD30L antibody or antigen binding fragment thereof at a concentration from about 75 mg/ml to about 275 mg/ml. In some embodiments, the pharmaceutical composition comprises an anti-CD30L antibody or antigen binding fragment thereof at a concentration from about 75 mg/ml to about 250 mg/ml. In some embodiments, the pharmaceutical composition comprises an anti-CD30L antibody or antigen binding fragment thereof at a concentration from about 75 mg/ml to about 225 mg/ml. In some embodiments, the pharmaceutical composition comprises an anti-CD30L antibody or antigen binding fragment thereof at a concentration from about 75 mg/ml to about 200 mg/ml. In some embodiments, the pharmaceutical composition comprises an anti-CD30L antibody or antigen binding fragment thereof at a concentration from about 75 mg/ml to about 175 mg/ml. In some embodiments, the pharmaceutical composition comprises an anti-CD30L antibody or antigen binding fragment thereof at a concentration from about 75 mg/ml to about 150 mg/ml. In some embodiments, the pharmaceutical composition comprises an anti-CD30L antibody or antigen binding fragment thereof at a concentration from about 75 mg/ml to about 125 mg/ml. In some embodiments, the pharmaceutical composition comprises an anti-CD30L antibody or antigen binding fragment thereof at a concentration from about 75 mg/ml to about 100 mg/ml. In some embodiments, the pharmaceutical composition comprises an anti-CD30L antibody or antigen binding fragment thereof at a concentration from about 100 mg/ml to about 275 mg/ml. In some embodiments, the pharmaceutical composition comprises an anti-CD30L antibody or antigen binding fragment thereof at a concentration from about 100 mg/ml to about 250 mg/ml. In some embodiments, the pharmaceutical composition comprises an anti-CD30L antibody or antigen binding fragment thereof at a concentration from about 100 mg/ml to about 225 mg/ml. In some embodiments, the pharmaceutical composition comprises an anti-CD30L antibody or antigen binding fragment thereof at a concentration from about 100 mg/ml to about 200 mg/ml. In some embodiments, the pharmaceutical composition comprises an anti-CD30L antibody or antigen binding fragment thereof at a concentration from about 100 mg/ml to about 175 mg/ml. In some embodiments, the pharmaceutical composition comprises an anti-CD30L antibody or antigen binding fragment thereof at a concentration from about 100 mg/ml to about 150 mg/ml. In some embodiments, the pharmaceutical composition comprises an anti-CD30L antibody or antigen binding fragment thereof at a concentration from about 100 mg/ml to about 125 mg/ml. In some embodiments, the pharmaceutical composition comprises an anti-CD30L antibody or antigen binding fragment thereof at a concentration from about 125 mg/ml to about 275 mg/ml. In some embodiments, the pharmaceutical composition comprises an anti-CD30L antibody or antigen binding fragment thereof at a concentration from about 125 mg/ml to about 250 mg/ml. In some embodiments, the pharmaceutical composition comprises an anti-CD30L antibody or antigen binding fragment thereof at a concentration from about 125 mg/ml to about 225 mg/ml. In some embodiments, the pharmaceutical composition comprises an anti-CD30L antibody or antigen binding fragment thereof at a concentration from about 125 mg/ml to about 200 mg/ml. In some embodiments, the pharmaceutical composition comprises an anti-CD30L antibody or antigen binding fragment thereof at a concentration from about 125 mg/ml to about 175 mg/ml. In some embodiments, the pharmaceutical composition comprises an anti-CD30L antibody or antigen binding fragment thereof at a concentration from about 125 mg/ml to about 150 mg/ml. In some embodiments, the pharmaceutical composition comprises an anti-CD30L antibody or antigen binding fragment thereof at a concentration from about 150 mg/ml to about 275 mg/ml. In some embodiments, the pharmaceutical composition comprises an anti-CD30L antibody or antigen binding fragment thereof at a concentration from about 150 mg/ml to about 250 mg/ml. In some embodiments, the pharmaceutical composition comprises an anti-CD30L antibody or antigen binding fragment thereof at a concentration from about 150 mg/ml to about 225 mg/ml. In some embodiments, the pharmaceutical composition comprises an anti-CD30L antibody or antigen binding fragment thereof at a concentration from about 150 mg/ml to about 200 mg/ml. In some embodiments, the pharmaceutical composition comprises an anti-CD30L antibody or antigen binding fragment thereof at a concentration from about 150 mg/ml to about 175 mg/ml. In some embodiments, the pharmaceutical composition comprises an anti-CD30L antibody or antigen binding fragment thereof at a concentration from about 175 mg/ml to about 275 mg/ml. In some embodiments, the pharmaceutical composition comprises an anti-CD30L antibody or antigen binding fragment thereof at a concentration from about 175 mg/ml to about 250 mg/ml. In some embodiments, the pharmaceutical composition comprises an anti-CD30L antibody or antigen binding fragment thereof at a concentration from about 175 mg/ml to about 225 mg/ml. In some embodiments, the pharmaceutical composition comprises an anti-CD30L antibody or antigen binding fragment thereof at a concentration from about 175 mg/ml to about 200 mg/ml. In some embodiments, the pharmaceutical composition comprises an anti-CD30L antibody or antigen binding fragment thereof at a concentration from about 200 mg/ml to about 275 mg/ml. In some embodiments, the pharmaceutical composition comprises an anti-CD30L antibody or antigen binding fragment thereof at a concentration from about 200 mg/ml to about 250 mg/ml. In some embodiments, the pharmaceutical composition comprises an anti-CD30L antibody or antigen binding fragment thereof at a concentration from about 200 mg/ml to about 225 mg/ml. In some embodiments, the pharmaceutical composition comprises an anti-CD30L antibody or antigen binding fragment thereof at a concentration at least about 50 mg/mL. In some embodiments, the pharmaceutical composition comprises an anti-CD30L antibody or antigen binding fragment thereof at a concentration at least about 75 mg/ml. In some embodiments, the pharmaceutical composition comprises an anti-CD30L antibody or antigen binding fragment thereof at a concentration at least about 100 mg/ml. In some embodiments, the pharmaceutical composition comprises an anti-CD30L antibody or antigen binding fragment thereof at a concentration at least about 125 mg/ml. In some embodiments, the pharmaceutical composition comprises an anti-CD30L antibody or antigen binding fragment thereof at a concentration at least about 150 mg/ml. In some embodiments, the pharmaceutical composition comprises an anti-CD30L antibody or antigen binding fragment thereof at a concentration at least about 175 mg/ml. In some embodiments, the pharmaceutical composition comprises an anti-CD30L antibody or antigen binding fragment thereof at a concentration at least about 200 mg/ml. In some embodiments, the pharmaceutical composition comprises an anti-CD30L antibody or antigen binding fragment thereof at a concentration at least about 225 mg/ml. In some embodiments, the pharmaceutical composition comprises an anti-CD30L antibody or antigen binding fragment thereof at a concentration at least about 250 mg/ml.

### 4.5.2 Buffer in the Pharmaceutical Composition Comprising Anti-CD30L Antibody or Antigen Binding Fragment

Certain embodiments of the buffers in the pharmaceutical compositions comprising anti-CD30L provided in Section 4.5 are described and provided in in this Section 4.5.2. Accordingly, in certain embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition further comprises a buffer provided in this Section 4.5.2. In certain embodiments, provided herein is a pharmaceutical composition comprising an anti-CD30L antibody or antigen binding fragment thereof provided in Sections 2 and 4.1, wherein the pharmaceutical composition further comprises a buffer provided in this Section 4.5.2.

The antibodies of the pharmaceutical compositions provided herein can be formulated in a variety of buffers. In certain embodiments, the antibodies are formulated in a buffer selected from the group consisting of an acetate buffer, a histidine buffer, a succinate buffer, a citrate buffer, a phosphate buffer, and an arginine buffer. In some embodiments, the antibodies are formulated in an acetate buffer. In other embodiments, the antibodies are formulated in a histidine buffer. In certain embodiments, the antibodies are formulated in a succinate buffer. In another embodiment, the antibodies are formulated in a citrate buffer. In some embodiments, the antibodies are formulated in a phosphate buffer. In other embodiments, the antibodies are formulated in an arginine buffer. In some embodiments, the antibodies are formulated in more than one buffer selected from the group consisting of an acetate buffer, a histidine buffer, a succinate buffer, a citrate buffer, a phosphate buffer, and an arginine buffer. In certain embodiments, the antibodies are formulated in a mixture of two buffers selected from the group consisting of an acetate buffer, a histidine buffer, a succinate buffer, a citrate buffer, a phosphate buffer, and an arginine buffer. Exemplary mixtures of two buffers include, but are not limited to, a histidine-arginine buffer, a phosphate-citrate buffer, and a histidine-acetate buffer, etc.

The antibodies of the pharmaceutical compositions provided herein can be formulated in a range of pH values. In some embodiments, the pH of the buffer is from 4.0 to 8.0. In other embodiments, the pH of the buffer is from 4.0 to 7. In yet other embodiments, the pH of the buffer is from 5 to 6.5. In some embodiments, the pH of the buffer is from 5.9 to 6.1. In yet other embodiments, the pH of the buffer is from 5.8 to 6.2. In some embodiments, the pH of the buffer is from 5.7 to 6.3. In some embodiments, the pH of the buffer is from 5.6 to 6.4. In some embodiments, the pH of the buffer is from 5.5 to 6.5. In some embodiments, the pH of the buffer is from 4.9 to 5.1. In some embodiments, the pH of the buffer is from 5.4 to 5.6. In yet other embodiments, the pH of the buffer is from 5.3 to 5.7. In some embodiments, the pH of the buffer is from 5.2 to 5.8. In some embodiments, the pH of the buffer is from 5.1 to 5.9. In some embodiments, the pH of the buffer is from 5.0 to 6.0. In some embodiments, the pH of the buffer is from 6.4 to 6.6. In yet other embodiments, the pH of the buffer is from 6.3 to 6.7. In some embodiments, the pH of the buffer is from 6.2 to 6.8. In some embodiments, the pH of the buffer is from 6.1 to 6.9. In some embodiments, the pH of the buffer is from 6.0 to 7.0. In some embodiments, the pH of the buffer is from 4.9 to 5.1. In yet other embodiments, the pH of the buffer is from 4.8 to 5.2. In some embodiments, the pH of the buffer is from 4.7 to 5.3. In some embodiments, the pH of the buffer is from 4.6 to 5.4. In some embodiments, the pH of the buffer is from 4.5 to 5.5. In one embodiment, the pH of the buffer is about 4.5. In one embodiment, the pH of the buffer is about 4.6. In one embodiment, the pH of the buffer is about 4.7. In one embodiment, the pH of the buffer is about 4.8. In one embodiment, the pH of the buffer is about 4.9. In one embodiment, the pH of the buffer is about 5.0. In one embodiment, the pH of the buffer is about 5.1. In another embodiment, the pH of the buffer is about 5.2. In one embodiment, the pH of the buffer is about 5.3. In one embodiment, the pH of the buffer is about 5.4. In yet another embodiment, the pH of the buffer is about 5.5. In yet another embodiment, the pH of the buffer is about 5.6. In one embodiment, the pH of the buffer is about 5.7. In yet another embodiment, the pH of the buffer is about 5.8. In one embodiment, the pH of the buffer is about 5.9. In still another embodiment, the pH of the buffer is about 6.0. In one embodiment, the pH of the buffer is about 6.1. In one embodiment, the pH of the buffer is about 6.2. In one embodiment, the pH of the buffer is about 6.3. In one embodiment, the pH of the buffer is about 6.4. In one embodiment, the pH of the buffer is about 6.5. In one embodiment, the pH of the buffer is about 6.6. In one embodiment, the pH of the buffer is about 6.7. In one embodiment, the pH of the buffer is about 6.8. In one embodiment, the pH of the buffer is about 6.9. In one embodiment, the pH of the buffer is about 7.0. In one embodiment, the pH of the buffer is 4.5. In one embodiment, the pH of the buffer is 4.6. In one embodiment, the pH of the buffer is 4.7. In one embodiment, the pH of the buffer is 4.8. In one embodiment, the pH of the buffer is 4.9. In one embodiment, the pH of the buffer is 5.0. In one embodiment, the pH of the buffer is 5.1. In another embodiment, the pH of the buffer is 5.2. In one embodiment, the pH of the buffer is 5.3. In one embodiment, the pH of the buffer is 5.4. In yet another embodiment, the pH of the buffer is 5.5. In yet another embodiment, the pH of the buffer is 5.6. In one embodiment, the pH of the buffer is 5.7. In yet another embodiment, the pH of the buffer is 5.8. In one embodiment, the pH of the buffer is 5.9. In still another embodiment, the pH of the buffer is 6.0. In one embodiment, the pH of the buffer is 6.1. In one embodiment, the pH of the buffer is 6.2. In one embodiment, the pH of the buffer is 6.3. In one embodiment, the pH of the buffer is 6.4. In one embodiment, the pH of the buffer is 6.5. In one embodiment, the pH of the buffer is 6.6. In one embodiment, the pH of the buffer is 6.7. In one embodiment, the pH of the buffer is 6.8. In one embodiment, the pH of the buffer is 6.9. In one embodiment, the pH of the buffer is 7.0. The disclosure provides that the pH of the buffer in the pharmaceutical composition determines the pH of the pharmaceutical composition. Therefore, the pH of the buffer provided herein is provided as embodiment of the pH of the pharmaceutical composition. In one embodiment, the anti-CD30L provided herein can be formulated in a range of pH values described herein, for example in this paragraph, with acetate as the buffering agent. In one embodiment, the anti-CD30L provided herein can be formulated in a range of pH values described herein, for example in this paragraph, with histidine as the buffering agent. In one embodiment, the anti-CD30L provided herein can be formulated in a pharmaceutical composition in a range of pH values described herein, for example in this paragraph, with histidine as the buffering agent. In another embodiment, the anti-CD30L provided herein can be formulated in a pharmaceutical composition in a range of pH values described herein, for example in this paragraph, with succinate as the buffering agent. In a further embodiment, the anti-CD30L provided herein can be formulated in a pharmaceutical composition in a range of pH values described herein, for example in this paragraph, with citrate as the buffering agent. In yet another embodiment, the anti-CD30L provided herein can be formulated in a pharmaceutical composition in a range of pH values described herein, for example in this paragraph, with phosphate as the buffering agent. In yet another embodiment, the anti-CD30L provided herein can be formulated in a pharmaceutical composition in a range of pH values described herein, for example in this paragraph, with arginine as the buffering agent.

In certain embodiments, the pharmaceutical composition comprising an anti-CD30L antibody or antigen binding fragment comprise a buffer at a pH of 4.5 to 8.0. For instance, the pH of the buffer in the pharmaceutical composition is about 4.5 to about 8.0, about 4.5 to about 7.9, about 4.5 to about 7.8, about 4.5 to about 7.7, about 4.5 to about 7.6, about 4.5 to about 7.5, about 4.5 to about 7.4, about 4.5 to about 7.3, about 4.5 to about 7.2, about 4.5 to about 7.1, about 4.5 to about 7.0, about 4.5 to about 6.9, about 4.5 to about 6.8, about 4.5 to about 6.7, about 4.5 to about 6.6, about 4.5 to about 6.5, about 4.5 to about 6.4, about 4.5 to about 6.3, about 4.5 to about 6.2, about 4.5 to about 6.1, about 4.5 to about 6.0, about 4.5 to about 5.9, about 4.5 to about 5.8, about 4.5 to about 5.7, about 4.5 to about 5.6, about 4.5 to about 5.5, about 4.5 to about 5.4, about 4.5 to about 5.3, about 4.5 to about 5.2, about 4.5 to about 5.1, about 4.5 to about 5.0, about 4.6 to about 8.0, about 4.6 to about 7.9, about 4.6 to about 7.8, about 4.6 to about 7.7, about 4.6 to about 7.6, about 4.6 to about 7.5, about 4.6 to about 7.4, about 4.6 to about 7.3, about 4.6 to about 7.2, about 4.6 to about 7.1, about 4.6 to about 7.0, about 4.6 to about 6.9, about 4.6 to about 6.8, about 4.6 to about 6.7, about 4.6 to about 6.6, about 4.6 to about 6.5, about 4.6 to about 6.4, about 4.6 to about 6.3, about 4.6 to about 6.2, about 4.6 to about 6.1, about 4.6 to about 6.0, about 4.6 to about 5.9, about 4.6 to about 5.8, about 4.6 to about 5.7, about 4.6 to about 5.6, about 4.6 to about 5.5, about 4.6 to about 5.4, about 4.6 to about 5.3, about 4.6 to about 5.2, about 4.6 to about 5.1, about 4.6 to about 5.0, about 4.7 to about 8.0, about 4.7 to about 7.9, about 4.7 to about 7.8, about 4.7 to about 7.7, about 4.7 to about 7.6, about 4.7 to about 7.5, about 4.7 to about 7.4, about 4.7 to about 7.3, about 4.7 to about 7.2, about 4.7 to about 7.1, about 4.7 to about 7.0, about 4.7 to about 6.9, about 4.7 to about 6.8, about 4.7 to about 6.7, about 4.7 to about 6.6, about 4.7 to about 6.5, about 4.7 to about 6.4, about 4.7 to about 6.3, about 4.7 to about 6.2, about 4.7 to about 6.1, about 4.7 to about 6.0, about 4.7 to about 5.9, about 4.7 to about 5.8, about 4.7 to about 5.7, about 4.7 to about 5.6, about 4.7 to about 5.5, about 4.7 to about 5.4, about 4.7 to about 5.3, about 4.7 to about 5.2, about 4.7 to about 5.1, about 4.7 to about 5.0, about 4.8 to about 8.0, about 4.8 to about 7.9, about 4.8 to about 7.8, about 4.8 to about 7.7, about 4.8 to about 7.6, about 4.8 to about 7.5, about 4.8 to about 7.4, about 4.8 to about 7.3, about 4.8 to about 7.2, about 4.8 to about 7.1, about 4.8 to about 7.0, about 4.8 to about 6.9, about 4.8 to about 6.8, about 4.8 to about 6.7, about 4.8 to about 6.6, about 4.8 to about 6.5, about 4.8 to about 6.4, about 4.8 to about 6.3, about 4.8 to about 6.2, about 4.8 to about 6.1, about 4.8 to about 6.0, about 4.8 to about 5.9, about 4.8 to about 5.8, about 4.8 to about 5.7, about 4.8 to about 5.6, about 4.8 to about 5.5, about 4.8 to about 5.4, about 4.8 to about 5.3, about 4.8 to about 5.2, about 4.8 to about 5.1, about 4.8 to about 5.0, about 4.9 to about 8.0, about 4.9 to about 7.9, about 4.9 to about 7.8, about 4.9 to about 7.7, about 4.9 to about 7.6, about 4.9 to about 7.5, about 4.9 to about 7.4, about 4.9 to about 7.3, about 4.9 to about 7.2, about 4.9 to about 7.1, about 4.9 to about 7.0, about 4.9 to about 6.9, about 4.9 to about 6.8, about 4.9 to about 6.7, about 4.9 to about 6.6, about 4.9 to about 6.5, about 4.9 to about 6.4, about 4.9 to about 6.3, about 4.9 to about 6.2, about 4.9 to about 6.1, about 4.9 to about 6.0, about 4.9 to about 5.9, about 4.9 to about 5.8, about 4.9 to about 5.7, about 4.9 to about 5.6, about 4.9 to about 5.5, about 4.9 to about 5.4, about 4.9 to about 5.3, about 4.9 to about 5.2, about 4.9 to about 5.1, about 4.9 to about 5.0, about 5.0 to about 8.0, about 5.0 to about 7.9, about 5.0 to about 7.8, about 5.0 to about 7.7, about 5.0 to about 7.6, about 5.0 to about 7.5, about 5.0 to about 7.4, about 5.0 to about 7.3, about 5.0 to about 7.2, about 5.0 to about 7.1, about 5.0 to about 7.0, about 5.0 to about 6.9, about 5.0 to about 6.8, about 5.0 to about 6.7, about 5.0 to about 6.6, about 5.0 to about 6.5, about 5.0 to about 6.4, about 5.0 to about 6.3, about 5.0 to about 6.2, about 5.0 to about 6.1, about 5.0 to about 6.0, about 5.0 to about 5.9, about 5.0 to about 5.8, about 5.0 to about 5.7, about 5.0 to about 5.6, about 5.0 to about 5.5, about 5.0 to about 5.4, about 5.0 to about 5.3, about 5.0 to about 5.2, about 5.0 to about 5.1, about 5.1 to about 8.0, about 5.1 to about 7.9, about 5.1 to about 7.8, about 5.1 to about 7.7, about 5.1 to about 7.6, about 5.1 to about 7.5, about 5.1 to about 7.4, about 5.1 to about 7.3, about 5.1 to about 7.2, about 5.1 to about 7.1, about 5.1 to about 7.0, about 5.1 to about 6.9, about 5.1 to about 6.8, about 5.1 to about 6.7, about 5.1 to about 6.6, about 5.1 to about 6.5, about 5.1 to about 6.4, about 5.1 to about 6.3, about 5.1 to about 6.2, about 5.1 to about 6.1, about 5.1 to about 6.0, about 5.1 to about 5.9, about 5.1 to about 5.8, about 5.1 to about 5.7, about 5.1 to about 5.6, about 5.1 to about 5.5, about 5.1 to about 5.4, about 5.1 to about 5.3, about 5.1 to about 5.2, about 5.2 to about 8.0, about 5.2 to about 7.9, about 5.2 to about 7.8, about 5.2 to about 7.7, about 5.2 to about 7.6, about 5.2 to about 7.5, about 5.2 to about 7.4, about 5.2 to about 7.3, about 5.2 to about 7.2, about 5.2 to about 7.1, about 5.2 to about 7.0, about 5.2 to about 6.9, about 5.2 to about 6.8, about 5.2 to about 6.7, about 5.2 to about 6.6, about 5.2 to about 6.5, about 5.2 to about 6.4, about 5.2 to about 6.3, about 5.2 to about 6.2, about 5.2 to about 6.1, about 5.2 to about 6.0, about 5.2 to about 5.9, about 5.2 to about 5.8, about 5.2 to about 5.7, about 5.2 to about 5.6, about 5.2 to about 5.5, about 5.2 to about 5.4, about 5.2 to about 5.3, about 5.3 to about 8.0, about 5.3 to about 7.9, about 5.3 to about 7.8, about 5.3 to about 7.7, about 5.3 to about 7.6, about 5.3 to about 7.5, about 5.3 to about 7.4, about 5.3 to about 7.3, about 5.3 to about 7.2, about 5.3 to about 7.1, about 5.3 to about 7.0, about 5.3 to about 6.9, about 5.3 to about 6.8, about 5.3 to about 6.7, about 5.3 to about 6.6, about 5.3 to about 6.5, about 5.3 to about 6.4, about 5.3 to about 6.3, about 5.3 to about 6.2, about 5.3 to about 6.1, about 5.3 to about 6.0, about 5.3 to about 5.9, about 5.3 to about 5.8, about 5.3 to about 5.7, about 5.3 to about 5.6, about 5.3 to about 5.5, about 5.3 to about 5.4, about 5.4 to about 8.0, about 5.4 to about 7.9, about 5.4 to about 7.8, about 5.4 to about 7.7, about 5.4 to about 7.6, about 5.4 to about 7.5, about 5.4 to about 7.4, about 5.4 to about 7.3, about 5.4 to about 7.2, about 5.4 to about 7.1, about 5.4 to about 7.0, about 5.4 to about 6.9, about 5.4 to about 6.8, about 5.4 to about 6.7, about 5.4 to about 6.6, about 5.4 to about 6.5, about 5.4 to about 6.4, about 5.4 to about 6.3, about 5.4 to about 6.2, about 5.4 to about 6.1, about 5.4 to about 6.0, about 5.4 to about 5.9, about 5.4 to about 5.8, about 5.4 to about 5.7, about 5.4 to about 5.6, about 5.4 to about 5.5, about 5.5 to about 8.0, about 5.5 to about 7.9, about 5.5 to about 7.8, about 5.5 to about 7.7, about 5.5 to about 7.6, about 5.5 to about 7.5, about 5.5 to about 7.4, about 5.5 to about 7.3, about 5.5 to about 7.2, about 5.5 to about 7.1, about 5.5 to about 7.0, about 5.5 to about 6.9, about 5.5 to about 6.8, about 5.5 to about 6.7, about 5.5 to about 6.6, about 5.5 to about 6.5, about 5.5 to about 6.4, about 5.5 to about 6.3, about 5.5 to about 6.2, about 5.5 to about 6.1, about 5.5 to about 6.0, about 5.5 to about 5.9, about 5.5 to about 5.8, about 5.5 to about 5.7, about 5.5 to about 5.6, about 5.6 to about 8.0, about 5.6 to about 7.9, about 5.6 to about 7.8, about 5.6 to about 7.7, about 5.6 to about 7.6, about 5.6 to about 7.5, about 5.6 to about 7.4, about 5.6 to about 7.3, about 5.6 to about 7.2, about 5.6 to about 7.1, about 5.6 to about 7.0, about 5.6 to about 6.9, about 5.6 to about 6.8, about 5.6 to about 6.7, about 5.6 to about 6.6, about 5.6 to about 6.5, about 5.6 to about 6.4, about 5.6 to about 6.3, about 5.6 to about 6.2, about 5.6 to about 6.1, about 5.6 to about 6.0, about 5.6 to about 5.9, about 5.6 to about 5.8, about 5.6 to about 5.7, about 5.7 to about 8.0, about 5.7 to about 7.9, about 5.7 to about 7.8, about 5.7 to about 7.7, about 5.7 to about 7.6, about 5.7 to about 7.5, about 5.7 to about 7.4, about 5.7 to about 7.3, about 5.7 to about 7.2, about 5.7 to about 7.1, about 5.7 to about 7.0, about 5.7 to about 6.9, about 5.7 to about 6.8, about 5.7 to about 6.7, about 5.7 to about 6.6, about 5.7 to about 6.5, about 5.7 to about 6.4, about 5.7 to about 6.3, about 5.7 to about 6.2, about 5.7 to about 6.1, about 5.7 to about 6.0, about 5.7 to about 5.9, about 5.7 to about 5.8, about 5.8 to about 8.0, about 5.8 to about 7.9, about 5.8 to about 7.8, about 5.8 to about 7.7, about 5.8 to about 7.6, about 5.8 to about 7.5, about 5.8 to about 7.4, about 5.8 to about 7.3, about 5.8 to about 7.2, about 5.8 to about 7.1, about 5.8 to about 7.0, about 5.8 to about 6.9, about 5.8 to about 6.8, about 5.8 to about 6.7, about 5.8 to about 6.6, about 5.8 to about 6.5, about 5.8 to about 6.4, about 5.8 to about 6.3, about 5.8 to about 6.2, about 5.8 to about 6.1, about 5.8 to about 6.0, about 5.8 to about 5.9, about 5.9 to about 8.0, about 5.9 to about 7.9, about 5.9 to about 7.8, about 5.9 to about 7.7, about 5.9 to about 7.6, about 5.9 to about 7.5, about 5.9 to about 7.4, about 5.9 to about 7.3, about 5.9 to about 7.2, about 5.9 to about 7.1, about 5.9 to about 7.0, about 5.9 to about 6.9, about 5.9 to about 6.8, about 5.9 to about 6.7, about 5.9 to about 6.6, about 5.9 to about 6.5, about 5.9 to about 6.4, about 5.9 to about 6.3, about 5.9 to about 6.2, about 5.9 to about 6.1, about 5.9 to about 6.0, about 6.0 to about 8.0, about 6.0 to about 7.9, about 6.0 to about 7.8, about 6.0 to about 7.7, about 6.0 to about 7.6, about 6.0 to about 7.5, about 6.0 to about 7.4, about 6.0 to about 7.3, about 6.0 to about 7.2, about 6.0 to about 7.1, about 6.0 to about 7.0, about 6.0 to about 6.9, about 6.0 to about 6.8, about 6.0 to about 6.7, about 6.0 to about 6.6, about 6.0 to about 6.5, about 6.0 to about 6.4, about 6.0 to about 6.3, about 6.0 to about 6.2, about 6.0 to about 6.1, about 6.1 to about 8.0, about 6.1 to about 7.9, about 6.1 to about 7.8, about 6.1 to about 7.7, about 6.1 to about 7.6, about 6.1 to about 7.5, about 6.1 to about 7.4, about 6.1 to about 7.3, about 6.1 to about 7.2, about 6.1 to about 7.1, about 6.1 to about 7.0, about 6.1 to about 6.9, about 6.1 to about 6.8, about 6.1 to about 6.7, about 6.1 to about 6.6, about 6.1 to about 6.5, about 6.1 to about 6.4, about 6.1 to about 6.3, about 6.1 to about 6.2, about 6.2 to about 8.0, about 6.2 to about 7.9, about 6.2 to about 7.8, about 6.2 to about 7.7, about 6.2 to about 7.6, about 6.2 to about 7.5, about 6.2 to about 7.4, about 6.2 to about 7.3, about 6.2 to about 7.2, about 6.2 to about 7.1, about 6.2 to about 7.0, about 6.2 to about 6.9, about 6.2 to about 6.8, about 6.2 to about 6.7, about 6.2 to about 6.6, about 6.2 to about 6.5, about 6.2 to about 6.4, about 6.2 to about 6.3, about 6.3 to about 8.0, about 6.3 to about 7.9, about 6.3 to about 7.8, about 6.3 to about 7.7, about 6.3 to about 7.6, about 6.3 to about 7.5, about 6.3 to about 7.4, about 6.3 to about 7.3, about 6.3 to about 7.2, about 6.3 to about 7.1, about 6.3 to about 7.0, about 6.3 to about 6.9, about 6.3 to about 6.8, about 6.3 to about 6.7, about 6.3 to about 6.6, about 6.3 to about 6.5, about 6.3 to about 6.4, about 6.4 to about 8.0, about 6.4 to about 7.9, about 6.4 to about 7.8, about 6.4 to about 7.7, about 6.4 to about 7.6, about 6.4 to about 7.5, about 6.4 to about 7.4, about 6.4 to about 7.3, about 6.4 to about 7.2, about 6.4 to about 7.1, about 6.4 to about 7.0, about 6.4 to about 6.9, about 6.4 to about 6.8, about 6.4 to about 6.7, about 6.4 to about 6.6, about 6.4 to about 6.5, about 6.5 to about 8.0, about 6.5 to about 7.9, about 6.5 to about 7.8, about 6.5 to about 7.7, about 6.5 to about 7.6, about 6.5 to about 7.5, about 6.5 to about 7.4, about 6.5 to about 7.3, about 6.5 to about 7.2, about 6.5 to about 7.1, about 6.5 to about 7.0, about 6.5 to about 6.9, about 6.5 to about 6.8, about 6.5 to about 6.7, or about 6.5 to about 6.6. In one embodiment, the anti-CD30L provided herein can be formulated in a pharmaceutical composition in a range of pH values described herein, for example in this paragraph, with acetate as the buffering agent. In one embodiment, the anti-CD30L provided herein can be formulated in a pharmaceutical composition in a range of pH values described herein, for example in this paragraph, with histidine as the buffering agent. In another embodiment, the anti-CD30L provided herein can be formulated in a pharmaceutical composition in a range of pH values described herein, for example in this paragraph, with succinate as the buffering agent. In a further embodiment, the anti-CD30L provided herein can be formulated in a pharmaceutical composition in a range of pH values described herein, for example in this paragraph, with citrate as the buffering agent. In yet another embodiment, the anti-CD30L provided herein can be formulated in a pharmaceutical composition in a range of pH values described herein, for example in this paragraph, with phosphate as the buffering agent. In yet another embodiment, the anti-CD30L provided herein can be formulated in a pharmaceutical composition in a range of pH values described herein, for example in this paragraph, with arginine as the buffering agent.

The disclosure further provides that the anti-CD30L antibody or antigen binding fragment of the pharmaceutical compositions provided herein can be formulated for a range of pH provided herein including in this Section 4.5.2 with various buffer agents provided herein including in this Section 4.5.2, wherein the buffer agent is at a range of concentrations such as those described in this Section 4.5.2 (such as this paragraph and the next paragraph). In some embodiments, the pharmaceutical composition comprising the anti-CD30L provided herein contains 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12.5 mM, 15 mM, 17.5 mM, 20 mM, 25 mM, 30 mM, 35 mM, 40 mM, 50 mM, 60 mM, 70 mM, 80 mM, 90 mM, 100 mM, 150 mM, 200 mM, 300 mM, 400 mM, 500 mM, 1 M, or more buffer agent provided herein. In some embodiments, the pharmaceutical composition comprising the anti-CD30L provided herein comprises 1 to 5 mM, 1 to 10 mM, 1 to 15 mM, 1 to 20 mM, 1 to 25 mM, 1 to 30 mM, 1 to 35 mM, 1 to 40 mM, 1 to 45 mM, 1 to 50 mM, 1 to 60 mM, 1 to 70 mM, 1 to 80 mM, 1 to 90 mM, 1 to 100 mM, 5 to 10 mM, 5 to 15 mM, 5 to 20 mM, 5 to 25 mM, 5 to 30 mM, 5 to 35 mM, 5 to 40 mM, 5 to 45 mM, 5 to 50 mM, 5 to 60 mM, 5 to 70 mM, 5 to 80 mM, 5 to 90 mM, 5 to 100 mM, 10 to 15 mM, 10 to 20 mM, 10 to 25 mM, 10 to 30 mM, 10 to 35 mM, 10 to 40 mM, 10 to 45 mM, 10 to 50 mM, 10 to 60 mM, 10 to 70 mM, 10 to 80 mM, 10 to 90 mM, 10 to 100 mM, 15 to 20 mM, 15 to 25 mM, 15 to 30 mM, 15 to 35 mM, 15 to 40 mM, 15 to 45 mM, 15 to 50 mM, 15 to 60 mM, 15 to 70 mM, 15 to 80 mM, 15 to 90 mM, 15 to 100 mM, 20 to 25 mM, 20 to 30 mM, 20 to 35 mM, 20 to 40 mM, 20 to 45 mM, 20 to 50 mM, 20 to 60 mM, 20 to 70 mM, 20 to 80 mM, 20 to 90 mM, 20 to 100 mM, 30 to 35 mM, 30 to 40 mM, 30 to 45 mM, 30 to 50 mM, 30 to 60 mM, 30 to 70 mM, 30 to 80 mM, 30 to 90 mM, 30 to 100 mM, 40 to 45 mM, 40 to 50 mM, 40 to 60 mM, 40 to 70 mM, 40 to 80 mM, 40 to 90 mM, 40 to 100 mM, 50 to 60 mM, 50 to 70 mM, 50 to 80 mM, 50 to 90 mM, 50 to 100 mM, 60 to 70 mM, 60 to 80 mM, 60 to 90 mM, 60 to 100 mM, 70 to 80 mM, 70 to 90 mM, 70 to 100 mM, 80 to 90 mM, 80 to 100 mM, 90 to 100 mM, 1 to 500 mM, 100 to 500 mM, 1 mM to 1 M, 10 mM to 1 M, 50 mM to 1 M, 500 mM to 1 M, or more buffer agent provided herein. In some embodiments, the pharmaceutical composition comprising the anti-CD30L provided herein contains 0.1 mM-1 M buffer provided herein. In other embodiments, the pharmaceutical composition comprising the anti-CD30L provided herein contains 1-100 mM buffer provided herein. In other embodiments, the pharmaceutical composition comprising the anti-CD30L provided herein contains 1-50 mM buffer provided herein. In some embodiments, the pharmaceutical composition comprising the anti-CD30L provided herein contains 1-40 mM buffer provided herein. In some embodiments, the pharmaceutical composition comprising the anti-CD30L provided herein contains 1-30 mM buffer provided herein. In some embodiments, the pharmaceutical composition comprising the anti-CD30L provided herein contains 1-20 mM buffer provided herein. In some embodiments, the pharmaceutical composition comprising the anti-CD30L provided herein contains 1-10 mM buffer provided herein. In other embodiments, the pharmaceutical composition comprising the anti-CD30L provided herein contains 5-50 mM buffer provided herein. In some embodiments, the pharmaceutical composition comprising the anti-CD30L provided herein contains 5-40 mM buffer provided herein. In some embodiments, the pharmaceutical composition comprising the anti-CD30L provided herein contains 5-30 mM buffer provided herein. In some embodiments, the pharmaceutical composition comprising the anti-CD30L provided herein contains 5-20 mM buffer provided herein. In some embodiments, the pharmaceutical composition comprising the anti-CD30L provided herein contains 5-10 mM buffer provided herein. In other embodiments, the pharmaceutical composition comprising the anti-CD30L provided herein contains 10-50 mM buffer provided herein. In some embodiments, the pharmaceutical composition comprising the anti-CD30L provided herein contains 10-40 mM buffer provided herein. In some embodiments, the pharmaceutical composition comprising the anti-CD30L provided herein contains 10-30 mM buffer provided herein. In some embodiments, the pharmaceutical composition comprising the anti-CD30L provided herein contains 10-20 mM buffer provided herein. In other embodiments, the pharmaceutical composition comprising the anti-CD30L provided herein contains 20-50 mM buffer provided herein. In some embodiments, the pharmaceutical composition comprising the anti-CD30L provided herein contains 20-40 mM buffer provided herein. In some embodiments, the pharmaceutical composition comprising the anti-CD30L provided herein contains 20-30 mM buffer provided herein. In other embodiments, the pharmaceutical composition comprising the anti-CD30L provided herein contains 30-50 mM buffer provided herein. In some embodiments, the pharmaceutical composition comprising the anti-CD30L provided herein contains 30-40 mM buffer provided herein. In other embodiments, the pharmaceutical composition comprising the anti-CD30L provided herein contains 40-50 mM buffer provided herein. In some embodiments, the pharmaceutical composition comprising the anti-CD30L provided herein contains 5 mM buffer provided herein. In other embodiments, the pharmaceutical composition comprising the anti-CD30L provided herein contains 10 mM buffer provided herein. In some embodiments, the pharmaceutical composition comprising the anti-CD30L provided herein contains 15 mM buffer provided herein. In other embodiments, the pharmaceutical composition comprising the anti-CD30L provided herein contains 20 mM buffer provided herein. In some embodiments, the pharmaceutical composition comprising the anti-CD30L provided herein contains 25 mM buffer provided herein. In other embodiments, the pharmaceutical composition comprising the anti-CD30L provided herein contains 30 mM buffer provided herein. In some embodiments, the pharmaceutical composition comprising the anti-CD30L provided herein contains 35 mM buffer provided herein. In other embodiments, the pharmaceutical composition comprising the anti-CD30L provided herein contains 40 mM buffer provided herein. In some embodiments, the pharmaceutical composition comprising the anti-CD30L provided herein contains 45 mM buffer provided herein. In other embodiments, the pharmaceutical composition comprising the anti-CD30L provided herein contains 50 mM buffer provided herein. In one embodiment, the anti-CD30L provided herein can be formulated in a pharmaceutical composition in a range of pH values described herein, for example in this Section 4.5.2, with acetate as the buffering agent at a concentration described in this paragraph. In one embodiment, the anti-CD30L provided herein can be formulated in a pharmaceutical composition in a range of pH values described herein, for example in this Section 4.5.2, with histidine as the buffering agent at a concentration described in this paragraph. In one embodiment, the anti-CD30L provided herein can be formulated in a pharmaceutical composition in a range of pH values described herein, for example in this Section 4.5.2, with succinate as the buffering agent at a concentration described in this paragraph. In a further embodiment, the anti-CD30L provided herein can be formulated in a pharmaceutical composition in a range of pH values described herein, for example in this Section 4.5.2, with citrate as the buffering agent at a concentration described in this paragraph. In yet another embodiment, the anti-CD30L provided herein can be formulated in a pharmaceutical composition in a range of pH values described herein, for example in this Section 4.5.2, with phosphate as the buffering agent at a concentration described in this paragraph. In yet another embodiment, the anti-CD30L provided herein can be formulated in a pharmaceutical composition in a range of pH values described herein, for example in this Section 4.5.2, with arginine as the buffering agent at a concentration described in this paragraph.

Specifically, in one embodiment, the pharmaceutical composition having an anti-CD30L provided herein comprises an acetate buffer at a concentration of from 0.1 mM to 1M. In another embodiment, the pharmaceutical composition having an anti-CD30L provided herein comprises an acetate buffer at a concentration of from 0.1 mM to 100 mM. In one embodiment, the pharmaceutical composition having an anti-CD30L provided herein comprises an acetate buffer at a concentration of from 0.1 mM to 10 mM. In one embodiment, the pharmaceutical composition having an anti-CD30L provided herein comprises an acetate buffer at a concentration of from 1 mM to 100 mM. In one embodiment, the pharmaceutical composition having an anti-CD30L provided herein comprises an acetate buffer at a concentration of from 1 mM to 50 mM. In one embodiment, the pharmaceutical composition having an anti-CD30L provided herein comprises an acetate buffer at a concentration of from 1 mM to 40 mM. In one embodiment, the pharmaceutical composition having an anti-CD30L provided herein comprises an acetate buffer at a concentration of from 1 mM to 30 mM. In one embodiment, the pharmaceutical composition having an anti-CD30L provided herein comprises an acetate buffer at a concentration of from 1 mM to 20 mM. In one embodiment, the pharmaceutical composition having an anti-CD30L provided herein comprises an acetate buffer at a concentration of from 1 mM to 10 mM. In one embodiment, the pharmaceutical composition having an anti-CD30L provided herein comprises an acetate buffer at a concentration of from 10 mM to 50 mM. In one embodiment, the pharmaceutical composition having an anti-CD30L provided herein comprises an acetate buffer at a concentration of from 10 mM to 40 mM. In one embodiment, the pharmaceutical composition having an anti-CD30L provided herein comprises an acetate buffer at a concentration of from 10 mM to 30 mM. In one embodiment, the pharmaceutical composition having an anti-CD30L provided herein comprises an acetate buffer at a concentration of from 10 mM to 20 mM. In one embodiment, the pharmaceutical composition having an anti-CD30L provided herein comprises an acetate buffer at a concentration of from 5 mM to 25 mM. In one embodiment, the pharmaceutical composition having an anti-CD30L provided herein comprises an acetate buffer at a concentration of from 15 mM to 25 mM. In one embodiment, the pharmaceutical composition having an anti-CD30L provided herein comprises an acetate buffer at a concentration of 5 mM. In one embodiment, the pharmaceutical composition having an anti-CD30L provided herein comprises an acetate buffer at a concentration of 10 mM. In one embodiment, the pharmaceutical composition having an anti-CD30L provided herein comprises an acetate buffer at a concentration of 15 mM. In one embodiment, the pharmaceutical composition having an anti-CD30L provided herein comprises an acetate buffer at a concentration of 20 mM. In another embodiment, the pharmaceutical composition having an anti-CD30L provided herein comprises an acetate buffer at a concentration of 25 mM. In one embodiment, the pharmaceutical composition having an anti-CD30L provided herein comprises an acetate buffer at a concentration of 30 mM. In one embodiment, the pharmaceutical composition having an anti-CD30L provided herein comprises an acetate buffer at a concentration of 35 mM. In one embodiment, the pharmaceutical composition having an anti-CD30L provided herein comprises an acetate buffer at a concentration of 40 mM. In one embodiment, the pharmaceutical composition having an anti-CD30L provided herein comprises an acetate buffer at a concentration of 45 mM. In one embodiment, the pharmaceutical composition having an anti-CD30L provided herein comprises an acetate buffer at a concentration of 50 mM. In one embodiment, the pharmaceutical composition having an anti-CD30L provided herein comprises a succinate buffer at a concentration of from 0.1 mM to 1M. In another embodiment, the pharmaceutical composition having an anti-CD30L provided herein comprises a succinate buffer at a concentration of from 0.1 mM to 100 mM. In one embodiment, the pharmaceutical composition having an anti-CD30L provided herein comprises a succinate buffer at a concentration of from 0.1 mM to 10 mM. In one embodiment, the pharmaceutical composition having an anti-CD30L provided herein comprises a succinate buffer at a concentration of from 1 mM to 100 mM. In one embodiment, the pharmaceutical composition having an anti-CD30L provided herein comprises a succinate buffer at a concentration of from 1 mM to 50 mM. In one embodiment, the pharmaceutical composition having an anti-CD30L provided herein comprises a succinate buffer at a concentration of from 1 mM to 40 mM. In one embodiment, the pharmaceutical composition having an anti-CD30L provided herein comprises a succinate buffer at a concentration of from 1 mM to 30 mM. In one embodiment, the pharmaceutical composition having an anti-CD30L provided herein comprises a succinate buffer at a concentration of from 1 mM to 20 mM. In one embodiment, the pharmaceutical composition having an anti-CD30L provided herein comprises a succinate buffer at a concentration of from 1 mM to 10 mM. In one embodiment, the pharmaceutical composition having an anti-CD30L provided herein comprises a succinate buffer at a concentration of from 10 mM to 50 mM. In one embodiment, the pharmaceutical composition having an anti-CD30L provided herein comprises a succinate buffer at a concentration of from 10 mM to 40 mM. In one embodiment, the pharmaceutical composition having an anti-CD30L provided herein comprises a succinate buffer at a concentration of from 10 mM to 30 mM. In one embodiment, the pharmaceutical composition having an anti-CD30L provided herein comprises a succinate buffer at a concentration of from 10 mM to 20 mM. In one embodiment, the pharmaceutical composition having an anti-CD30L provided herein comprises a succinate buffer at a concentration of from 5 mM to 25 mM. In one embodiment, the pharmaceutical composition having an anti-CD30L provided herein comprises a succinate buffer at a concentration of from 15 mM to 25 mM. In one embodiment, the pharmaceutical composition having an anti-CD30L provided herein comprises a succinate buffer at a concentration of 5 mM. In one embodiment, the pharmaceutical composition having an anti-CD30L provided herein comprises a succinate buffer at a concentration of 10 mM. In one embodiment, the pharmaceutical composition having an anti-CD30L provided herein comprises a succinate buffer at a concentration of 15 mM. In one embodiment, the pharmaceutical composition having an anti-CD30L provided herein comprises a succinate buffer at a concentration of 20 mM. In another embodiment, the pharmaceutical composition having an anti-CD30L provided herein comprises a succinate buffer at a concentration of 25 mM. In one embodiment, the pharmaceutical composition having an anti-CD30L provided herein comprises a succinate buffer at a concentration of 30 mM. In one embodiment, the pharmaceutical composition having an anti-CD30L provided herein comprises a succinate buffer at a concentration of 35 mM. In one embodiment, the pharmaceutical composition having an anti-CD30L provided herein comprises a succinate buffer at a concentration of 40 mM. In one embodiment, the pharmaceutical composition having an anti-CD30L provided herein comprises a succinate buffer at a concentration of 45 mM. In one embodiment, the pharmaceutical composition having an anti-CD30L provided herein comprises a succinate buffer at a concentration of 50 mM. In one embodiment, the pharmaceutical composition having an anti-CD30L provided herein comprises a histidine buffer at a concentration of from 0.1 mM to 1M. In another embodiment, the pharmaceutical composition having an anti-CD30L provided herein comprises a histidine buffer at a concentration of from 0.1 mM to 100 mM. In one embodiment, the pharmaceutical composition having an anti-CD30L provided herein comprises a histidine buffer at a concentration of from 0.1 mM to 10 mM. In one embodiment, the pharmaceutical composition having an anti-CD30L provided herein comprises a histidine buffer at a concentration of from 1 mM to 100 mM. In one embodiment, the pharmaceutical composition having an anti-CD30L provided herein comprises a histidine buffer at a concentration of from 1 mM to 50 mM. In one embodiment, the pharmaceutical composition having an anti-CD30L provided herein comprises a histidine buffer at a concentration of from 1 mM to 40 mM. In one embodiment, the pharmaceutical composition having an anti-CD30L provided herein comprises a histidine buffer at a concentration of from 1 mM to 30 mM. In one embodiment, the pharmaceutical composition having an anti-CD30L provided herein comprises a histidine buffer at a concentration of from 1 mM to 20 mM. In one embodiment, the pharmaceutical composition having an anti-CD30L provided herein comprises a histidine buffer at a concentration of from 1 mM to 10 mM. In one embodiment, the pharmaceutical composition having an anti-CD30L provided herein comprises a histidine buffer at a concentration of from 10 mM to 50 mM. In one embodiment, the pharmaceutical composition having an anti-CD30L provided herein comprises a histidine buffer at a concentration of from 10 mM to 40 mM. In one embodiment, the pharmaceutical composition having an anti-CD30L provided herein comprises a histidine buffer at a concentration of from 10 mM to 30 mM. In one embodiment, the pharmaceutical composition having an anti-CD30L provided herein comprises a histidine buffer at a concentration of from 10 mM to 20 mM. In one embodiment, the pharmaceutical composition having an anti-CD30L provided herein comprises a histidine buffer at a concentration of from 5 mM to 25 mM. In one embodiment, the pharmaceutical composition having an anti-CD30L provided herein comprises a histidine buffer at a concentration of from 15 mM to 25 mM. In one embodiment, the pharmaceutical composition having an anti-CD30L provided herein comprises a histidine buffer at a concentration of 5 mM. In one embodiment, the pharmaceutical composition having an anti-CD30L provided herein comprises a histidine buffer at a concentration of 10 mM. In one embodiment, the pharmaceutical composition having an anti-CD30L provided herein comprises a histidine buffer at a concentration of 15 mM. In one embodiment, the pharmaceutical composition having an anti-CD30L provided herein comprises a histidine buffer at a concentration of 20 mM. In another embodiment, the pharmaceutical composition having an anti-CD30L provided herein comprises a histidine buffer at a concentration of 25 mM. In one embodiment, the pharmaceutical composition having an anti-CD30L provided herein comprises a histidine buffer at a concentration of 30 mM. In one embodiment, the pharmaceutical composition having an anti-CD30L provided herein comprises a histidine buffer at a concentration of 35 mM. In one embodiment, the pharmaceutical composition having an anti-CD30L provided herein comprises a histidine buffer at a concentration of 40 mM. In one embodiment, the pharmaceutical composition having an anti-CD30L provided herein comprises a histidine buffer at a concentration of 45 mM. In one embodiment, the pharmaceutical composition having an anti-CD30L provided herein comprises a histidine buffer at a concentration of 50 mM. In one embodiment, the pharmaceutical composition having an anti-CD30L provided herein comprises a phosphate buffer at a concentration of from 0.1 mM to 1M. In another embodiment, the pharmaceutical composition having an anti-CD30L provided herein comprises a phosphate buffer at a concentration of from 0.1 mM to 100 mM. In one embodiment, the pharmaceutical composition having an anti-CD30L provided herein comprises a phosphate buffer at a concentration of from 0.1 mM to 10 mM. In one embodiment, the pharmaceutical composition having an anti-CD30L provided herein comprises a phosphate buffer at a concentration of from 1 mM to 100 mM. In one embodiment, the pharmaceutical composition having an anti-CD30L provided herein comprises a phosphate buffer at a concentration of from 1 mM to 50 mM. In one embodiment, the pharmaceutical composition having an anti-CD30L provided herein comprises a phosphate buffer at a concentration of from 1 mM to 40 mM. In one embodiment, the pharmaceutical composition having an anti-CD30L provided herein comprises a phosphate buffer at a concentration of from 1 mM to 30 mM. In one embodiment, the pharmaceutical composition having an anti-CD30L provided herein comprises a phosphate buffer at a concentration of from 1 mM to 20 mM. In one embodiment, the pharmaceutical composition having an anti-CD30L provided herein comprises a phosphate buffer at a concentration of from 1 mM to 10 mM. In one embodiment, the pharmaceutical composition having an anti-CD30L provided herein comprises a phosphate buffer at a concentration of from 10 mM to 50 mM. In one embodiment, the pharmaceutical composition having an anti-CD30L provided herein comprises a phosphate buffer at a concentration of from 10 mM to 40 mM. In one embodiment, the pharmaceutical composition having an anti-CD30L provided herein comprises a phosphate buffer at a concentration of from 10 mM to 30 mM. In one embodiment, the pharmaceutical composition having an anti-CD30L provided herein comprises a phosphate buffer at a concentration of from 10 mM to 20 mM. In one embodiment, the pharmaceutical composition having an anti-CD30L provided herein comprises a phosphate buffer at a concentration of from 5 mM to 25 mM. In one embodiment, the pharmaceutical composition having an anti-CD30L provided herein comprises a phosphate buffer at a concentration of from 15 mM to 25 mM. In one embodiment, the pharmaceutical composition having an anti-CD30L provided herein comprises a phosphate buffer at a concentration of 5 mM. In one embodiment, the pharmaceutical composition having an anti-CD30L provided herein comprises a phosphate buffer at a concentration of 10 mM. In one embodiment, the pharmaceutical composition having an anti-CD30L provided herein comprises a phosphate buffer at a concentration of 15 mM. In one embodiment, the pharmaceutical composition having an anti-CD30L provided herein comprises a phosphate buffer at a concentration of 20 mM. In another embodiment, the pharmaceutical composition having an anti-CD30L provided herein comprises a phosphate buffer at a concentration of 25 mM. In one embodiment, the pharmaceutical composition having an anti-CD30L provided herein comprises a phosphate buffer at a concentration of 30 mM. In one embodiment, the pharmaceutical composition having an anti-CD30L provided herein comprises a phosphate buffer at a concentration of 35 mM. In one embodiment, the pharmaceutical composition having an anti-CD30L provided herein comprises a phosphate buffer at a concentration of 40 mM. In one embodiment, the pharmaceutical composition having an anti-CD30L provided herein comprises a phosphate buffer at a concentration of 45 mM. In one embodiment, the pharmaceutical composition having an anti-CD30L provided herein comprises a phosphate buffer at a concentration of 50 mM. In one embodiment, the pharmaceutical composition having an anti-CD30L provided herein comprises a citrate buffer at a concentration of from 0.1 mM to 1M. In another embodiment, the pharmaceutical composition having an anti-CD30L provided herein comprises a citrate buffer at a concentration of from 0.1 mM to 100 mM. In one embodiment, the pharmaceutical composition having an anti-CD30L provided herein comprises a citrate buffer at a concentration of from 0.1 mM to 10 mM. In one embodiment, the pharmaceutical composition having an anti-CD30L provided herein comprises a citrate buffer at a concentration of from 1 mM to 100 mM. In one embodiment, the pharmaceutical composition having an anti-CD30L provided herein comprises a citrate buffer at a concentration of from 1 mM to 50 mM. In one embodiment, the pharmaceutical composition having an anti-CD30L provided herein comprises a citrate buffer at a concentration of from 1 mM to 40 mM. In one embodiment, the pharmaceutical composition having an anti-CD30L provided herein comprises a citrate buffer at a concentration of from 1 mM to 30 mM. In one embodiment, the pharmaceutical composition having an anti-CD30L provided herein comprises a citrate buffer at a concentration of from 1 mM to 20 mM. In one embodiment, the pharmaceutical composition having an anti-CD30L provided herein comprises a citrate buffer at a concentration of from 1 mM to 10 mM. In one embodiment, the pharmaceutical composition having an anti-CD30L provided herein comprises a citrate buffer at a concentration of from 10 mM to 50 mM. In one embodiment, the pharmaceutical composition having an anti-CD30L provided herein comprises a citrate buffer at a concentration of from 10 mM to 40 mM. In one embodiment, the pharmaceutical composition having an anti-CD30L provided herein comprises a citrate buffer at a concentration of from 10 mM to 30 mM. In one embodiment, the pharmaceutical composition having an anti-CD30L provided herein comprises a citrate buffer at a concentration of from 10 mM to 20 mM. In one embodiment, the pharmaceutical composition having an anti-CD30L provided herein comprises a citrate buffer at a concentration of from 5 mM to 25 mM. In one embodiment, the pharmaceutical composition having an anti-CD30L provided herein comprises a citrate buffer at a concentration of from 15 mM to 25 mM. In one embodiment, the pharmaceutical composition having an anti-CD30L provided herein comprises a citrate buffer at a concentration of 5 mM. In one embodiment, the pharmaceutical composition having an anti-CD30L provided herein comprises a citrate buffer at a concentration of 10 mM. In one embodiment, the pharmaceutical composition having an anti-CD30L provided herein comprises a citrate buffer at a concentration of 15 mM. In one embodiment, the pharmaceutical composition having an anti-CD30L provided herein comprises a citrate buffer at a concentration of 20 mM. In another embodiment, the pharmaceutical composition having an anti-CD30L provided herein comprises a citrate buffer at a concentration of 25 mM. In one embodiment, the pharmaceutical composition having an anti-CD30L provided herein comprises a citrate buffer at a concentration of 30 mM. In one embodiment, the pharmaceutical composition having an anti-CD30L provided herein comprises a citrate buffer at a concentration of 35 mM. In one embodiment, the pharmaceutical composition having an anti-CD30L provided herein comprises a citrate buffer at a concentration of 40 mM. In one embodiment, the pharmaceutical composition having an anti-CD30L provided herein comprises a citrate buffer at a concentration of 45 mM. In one embodiment, the pharmaceutical composition having an anti-CD30L provided herein comprises a citrate buffer at a concentration of 50 mM. As is clear from the descriptions above, the range of concentration of the buffer agent and the type of the buffer agent provided herein can be used to formulate the pharmaceutical compositing having an anti-CD30L for a range of pH described herein including in this Section 4.5.2 (such as the preceding paragraphs of this Section 4.5.2). Accordingly, the pharmaceutical composition having an anti-CD30L provided herein can be formulated for a range of pH provided herein including in this Section 4.5.2 with various buffer agents provided herein including in this Section 4.5.2 (such as in this and the preceding paragraphs), wherein the buffer agent is at a range of concentrations described in this Section 4.5.2 (such as this and the preceding paragraphs).

### 4.5.3 Excipient in the Pharmaceutical Composition Comprising Anti-CD30L Antibody or Antigen Binding Fragment

Certain embodiments of the excipients in the pharmaceutical compositions comprising anti-CD30L provided in Section 4.5 are described and provided in this Section 4.5.3. Accordingly, in certain embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition comprises a buffer provided in Section 4.5.2, and wherein the pharmaceutical composition comprises a pharmaceutically acceptable excipient provided in this Section 4.5.3. In some embodiments, provided herein is a pharmaceutical composition comprising an anti-CD30L antibody or antigen binding fragment thereof provided in Sections 2 and 4.1, wherein the pharmaceutical composition comprises a pharmaceutically acceptable excipient provided in this Section 4.5.3. In certain embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition comprises a pharmaceutically acceptable excipient provided in this Section 4.5.3. In certain embodiments, provided herein is a pharmaceutical composition comprising an anti-CD30L antibody or antigen binding fragment thereof provided in Sections 2 and 4.1, wherein the pharmaceutical composition comprises a buffer provided in Section 4.5.2, and wherein the pharmaceutical composition comprises a pharmaceutically acceptable excipient provided in this Section 4.5.3.

In various embodiments of the pharmaceutical composition provided herein, the pharmaceutical composition further comprises a pharmaceutically acceptable excipient, wherein the pharmaceutically acceptable excipient is arginine, arginine hydrochloride (arginine HCl), proline, a polyol, sodium chloride, glycine, lysine, lysine-hydrochloride, arginine glutamate, potassium chloride, magnesium chloride, calcium chloride, or a combination thereof. In one embodiment, the pharmaceutical composition having an anti-CD30L provided herein further comprises a pharmaceutically acceptable excipient, wherein the pharmaceutically acceptable excipient is arginine. In one embodiment, the pharmaceutical composition having an anti-CD30L provided herein further comprises a pharmaceutically acceptable excipient, wherein the pharmaceutically acceptable excipient is arginine hydrochloride (arginine HCl). In one embodiment, the pharmaceutical composition having an anti-CD30L provided herein further comprises a pharmaceutically acceptable excipient, wherein the pharmaceutically acceptable excipient is proline. In one embodiment, the pharmaceutical composition having an anti-CD30L provided herein further comprises a pharmaceutically acceptable excipient, wherein the pharmaceutically acceptable excipient is a polyol. In one embodiment, the pharmaceutical composition having an anti-CD30L provided herein further comprises a pharmaceutically acceptable excipient, wherein the pharmaceutically acceptable excipient is sodium chloride. In one embodiment, the pharmaceutical composition having an anti-CD30L provided herein further comprises a pharmaceutically acceptable excipient, wherein the pharmaceutically acceptable excipient is glycine. In one embodiment, the pharmaceutical composition having an anti-CD30L provided herein further comprises a pharmaceutically acceptable excipient, wherein the pharmaceutically acceptable excipient is lysine. In one embodiment, the pharmaceutical composition having an anti-CD30L provided herein further comprises a pharmaceutically acceptable excipient, wherein the pharmaceutically acceptable excipient is lysine-hydrochloride. In one embodiment, the pharmaceutical composition having an anti-CD30L provided herein further comprises a pharmaceutically acceptable excipient, wherein the pharmaceutically acceptable excipient is arginine glutamate. In one embodiment, the pharmaceutical composition having an anti-CD30L provided herein further comprises a pharmaceutically acceptable excipient, wherein the pharmaceutically acceptable excipient is potassium chloride. In one embodiment, the pharmaceutical composition having an anti-CD30L provided herein further comprises a pharmaceutically acceptable excipient, wherein the pharmaceutically acceptable excipient is magnesium chloride. In one embodiment, the pharmaceutical composition having an anti-CD30L provided herein further comprises a pharmaceutically acceptable excipient, wherein the pharmaceutically acceptable excipient is calcium chloride. In some embodiments, the pharmaceutical composition having an anti-CD30L provided herein contains about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, about 10%, about 12.5%, about 15%, about 17.5%, about 20%, about 25%, about 30% (w/v), or more pharmaceutically acceptable excipient. In certain embodiments, the pharmaceutical composition of the antibodies provided herein contains 1-2.5%, 1.5-2.5%, 1-5%, 2.5-7.5%, 4-6%, 4.5-5.5%, 1-10%, 5-10%, 10-15%, 15-20%, 20-25%, 25-30%, 30-35%, 35-40%, (w/v), or more pharmaceutically acceptable excipient. In one embodiment, the concentration of the pharmaceutically acceptable excipient is from 1% to 2.5% (w/v). In one embodiment, the concentration of the pharmaceutically acceptable excipient is from 1.5% to 2.5% (w/v). In one embodiment, the concentration of the pharmaceutically acceptable excipient is from 1% to 5% (w/v). In one embodiment, the concentration of the pharmaceutically acceptable excipient is from 1% to 10% (w/v). In one embodiment, the concentration of the pharmaceutically acceptable excipient is from 2.5% to 7.5% (w/v). In one embodiment, the concentration of the pharmaceutically acceptable excipient is from 4.5% to 5.5% (w/v). In one embodiment, the concentration of the pharmaceutically acceptable excipient is from 5% to 10% (w/v). In one embodiment, the concentration of the pharmaceutically acceptable excipient is from 8.5% to 9.5% (w/v). In one embodiment, the concentration of the pharmaceutically acceptable excipient is about 1% (w/v). In one embodiment, the concentration of the pharmaceutically acceptable excipient is about 1.1% (w/v). In one embodiment, the concentration of the pharmaceutically acceptable excipient is about 1.2% (w/v). In one embodiment, the concentration of the pharmaceutically acceptable excipient is about 1.3% (w/v). In one embodiment, the concentration of the pharmaceutically acceptable excipient is about 1.4% (w/v). In one embodiment, the concentration of the pharmaceutically acceptable excipient is about 1.5% (w/v). In one embodiment, the concentration of the pharmaceutically acceptable excipient is about 1.6% (w/v). In one embodiment, the concentration of the pharmaceutically acceptable excipient is about 1.7% (w/v). In one embodiment, the concentration of the pharmaceutically acceptable excipient is about 1.8% (w/v). In one embodiment, the concentration of the pharmaceutically acceptable excipient is about 1.9% (w/v). In one embodiment, the concentration of the pharmaceutically acceptable excipient is about 2.0% (w/v). In one embodiment, the concentration of the pharmaceutically acceptable excipient is about 2.1% (w/v). In one embodiment, the concentration of the pharmaceutically acceptable excipient is about 2.2% (w/v). In one embodiment, the concentration of the pharmaceutically acceptable excipient is about 2.3% (w/v). In one embodiment, the concentration of the pharmaceutically acceptable excipient is about 2.4% (w/v). In one embodiment, the concentration of the pharmaceutically acceptable excipient is about 2.5% (w/v). In one embodiment, the concentration of the pharmaceutically acceptable excipient is about 2.6% (w/v). In one embodiment, the concentration of the pharmaceutically acceptable excipient is about 2.7% (w/v). In one embodiment, the concentration of the pharmaceutically acceptable excipient is about 2.8% (w/v). In one embodiment, the concentration of the pharmaceutically acceptable excipient is about 2.9% (w/v). In one embodiment, the concentration of the pharmaceutically acceptable excipient is about 3.0% (w/v). In one embodiment, the concentration of the pharmaceutically acceptable excipient is about 4% (w/v). In one embodiment, the concentration of the pharmaceutically acceptable excipient is about 4.1% (w/v). In one embodiment, the concentration of the pharmaceutically acceptable excipient is about 4.2% (w/v). In one embodiment, the concentration of the pharmaceutically acceptable excipient is about 4.3% (w/v). In one embodiment, the concentration of the pharmaceutically acceptable excipient is about 4.4% (w/v). In one embodiment, the concentration of the pharmaceutically acceptable excipient is about 4.5% (w/v). In one embodiment, the concentration of the pharmaceutically acceptable excipient is about 4.6% (w/v). In one embodiment, the concentration of the pharmaceutically acceptable excipient is about 4.7% (w/v). In one embodiment, the concentration of the pharmaceutically acceptable excipient is about 4.8% (w/v). In one embodiment, the concentration of the pharmaceutically acceptable excipient is about 4.9% (w/v). In one embodiment, the concentration of the pharmaceutically acceptable excipient is about 5% (w/v). In one embodiment, the concentration of the pharmaceutically acceptable excipient is about 5.1% (w/v). In one embodiment, the concentration of the pharmaceutically acceptable excipient is about 5.2% (w/v). In one embodiment, the concentration of the pharmaceutically acceptable excipient is about 5.3% (w/v). In one embodiment, the concentration of the pharmaceutically acceptable excipient is about 5.4% (w/v). In one embodiment, the concentration of the pharmaceutically acceptable excipient is about 5.5% (w/v). In one embodiment, the concentration of the pharmaceutically acceptable excipient is about 5.6% (w/v). In one embodiment, the concentration of the pharmaceutically acceptable excipient is about 5.7% (w/v). In one embodiment, the concentration of the pharmaceutically acceptable excipient is about 5.8% (w/v). In one embodiment, the concentration of the pharmaceutically acceptable excipient is about 5.9% (w/v). In one embodiment, the concentration of the pharmaceutically acceptable excipient is about 6% (w/v). In one embodiment, the concentration of the pharmaceutically acceptable excipient is about 7% (w/v). In one embodiment, the concentration of the pharmaceutically acceptable excipient is about 8% (w/v). In another embodiment, the concentration of the pharmaceutically acceptable excipient is about 9.5% (w/v). In one embodiment, the concentration of the pharmaceutically acceptable excipient is about 9.4% (w/v). In one embodiment, the concentration of the pharmaceutically acceptable excipient is about 9.3% (w/v). In one embodiment, the concentration of the pharmaceutically acceptable excipient is about 9.2% (w/v). In one embodiment, the concentration of the pharmaceutically acceptable excipient is about 9.1% (w/v). In one embodiment, the concentration of the pharmaceutically acceptable excipient is about 9.0% (w/v). In one embodiment, the concentration of the pharmaceutically acceptable excipient is about 8.9% (w/v). In one embodiment, the concentration of the pharmaceutically acceptable excipient is about 8.8% (w/v). In one embodiment, the concentration of the pharmaceutically acceptable excipient is about 8.7% (w/v). In one embodiment, the concentration of the pharmaceutically acceptable excipient is about 8.6% (w/v). In one embodiment, the concentration of the pharmaceutically acceptable excipient is about 8.5% (w/v). In some embodiments, the pharmaceutical composition of the antibodies provided herein contains about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 245 mM, about 250 mM, about 255 mM, about 260 mM, about 265 mM, about 270 mM, about 275 mM, about 280 mM, about 285 mM, about 290 mM, about 320 mM, about 350 mM, about 380 mM, about 410 mM, about 440 mM, about 470 mM, about 500 mM, about 550 mM, about 600 mM, about 700 mM, about 800 mM, about 900 mM, about 1 M, about 1.1 M, about 1.2 M, about 1.3 M, about 1.4 M, about 1.5 M, or more pharmaceutically acceptable excipient. In certain embodiments, the pharmaceutical composition of the antibodies provided herein contains 20-200 mM, 20-190 mM, 20-180 mM, 20-170 mM, 20-160 mM, 20-150 mM, 20-140 mM, 20-130 mM, 20-120 mM, 20-110 mM, 20-100 mM, 20-90 mM, 20-80 mM, 20-70 mM, 20-60 mM, 20-50 mM, 30-200 mM, 30-190 mM, 30-180 mM, 30-170 mM, 30-160 mM, 30-150 mM, 30-140 mM, 30-130 mM, 30-120 mM, 30-110 mM, 30-100 mM, 30-90 mM, 30-80 mM, 30-70 mM, 30-60 mM, 30-50 mM, 40-200 mM, 40-190 mM, 40-180 mM, 40-170 mM, 40-160 mM, 40-150 mM, 40-140 mM, 40-130 mM, 40-120 mM, 40-110 mM, 40-100 mM, 40-90 mM, 40-80 mM, 40-70 mM, 40-60 mM, 40-50 mM, 50-200 mM, 50-190 mM, 50-180 mM, 50-170 mM, 50-160 mM, 50-150 mM, 50-140 mM, 50-130 mM, 50-120 mM, 50-110 mM, 50-100 mM, 50-90 mM, 50-80 mM, 50-70 mM, 50-60 mM, 60-200 mM, 60-190 mM, 60-180 mM, 60-170 mM, 60-160 mM, 60-150 mM, 60-140 mM, 60-130 mM, 60-120 mM, 60-110 mM, 60-100 mM, 60-90 mM, 60-80 mM, 60-70 mM, 70-200 mM, 70-190 mM, 70-180 mM, 70-170 mM, 70-160 mM, 70-150 mM, 70-140 mM, 70-130 mM, 70-120 mM, 70-110 mM, 70-100 mM, 70-90 mM, 70-80 mM, 80-200 mM, 80-190 mM, 80-180 mM, 80-170 mM, 80-160 mM, 80-150 mM, 80-140 mM, 80-130 mM, 80-120 mM, 80-110 mM, 80-100 mM, 80-90 mM, 90-200 mM, 90-190 mM, 90-180 mM, 90-170 mM, 90-160 mM, 90-150 mM, 90-140 mM, 90-130 mM, 90-120 mM, 90-110 mM, 90-100 mM, 100-200 mM, 100-190 mM, 100-180 mM, 100-170 mM, 100-160 mM, 100-150 mM, 100-140 mM, 100-130 mM, 100-120 mM, 100-110 mM, 100-300 mM, 150-300 mM, 300-450 mM, 450-600 mM, 600-750 mM, 750-900 mM, 900 mM-1.05 M, 1.05-1.2 M, 1.2-1.35 M, 1.35-1.5M, or more pharmaceutically acceptable excipient. In one embodiment, the concentration of the pharmaceutically acceptable excipient is from 50 to 150 mM. In one embodiment, the concentration of the pharmaceutically acceptable excipient is from 60 to 140 mM. In one embodiment, the concentration of the pharmaceutically acceptable excipient is from 70 to 130 mM. In one embodiment, the concentration of the pharmaceutically acceptable excipient is from 80 to 120 mM. In one embodiment, the concentration of the pharmaceutically acceptable excipient is from 90 to 110 mM. In one embodiment, the concentration of the pharmaceutically acceptable excipient is from 100 to 200 mM. In one embodiment, the concentration of the pharmaceutically acceptable excipient is from 110 to 190 mM. In one embodiment, the concentration of the pharmaceutically acceptable excipient is from 120 to 180 mM. In one embodiment, the concentration of the pharmaceutically acceptable excipient is from 130 to 170 mM. In one embodiment, the concentration of the pharmaceutically acceptable excipient is from 140 to 160 mM. In one embodiment, the concentration of the pharmaceutically acceptable excipient is from 150 to 300 mM. In one embodiment, the concentration of the pharmaceutically acceptable excipient is from 245 mM to 275 mM. In another embodiment, the concentration of the pharmaceutically acceptable excipient is about 290 mM. In one embodiment, the concentration of the pharmaceutically acceptable excipient is about 285 mM. In one embodiment, the concentration of the pharmaceutically acceptable excipient is about 280 mM. In one embodiment, the concentration of the pharmaceutically acceptable excipient is about 275 mM. In one embodiment, the concentration of the pharmaceutically acceptable excipient is about 270 mM. In one embodiment, the concentration of the pharmaceutically acceptable excipient is about 265 mM. In one embodiment, the concentration of the pharmaceutically acceptable excipient is about 260 mM. In one embodiment, the concentration of the pharmaceutically acceptable excipient is about 255 mM. In one embodiment, the concentration of the pharmaceutically acceptable excipient is about 250 mM. In one embodiment, the concentration of the pharmaceutically acceptable excipient is about 245 mM. In one embodiment, the concentration of the pharmaceutically acceptable excipient is about 240 mM. In one embodiment, the concentration of the pharmaceutically acceptable excipient is about 235 mM. In one embodiment, the concentration of the pharmaceutically acceptable excipient is about 230 mM. In one embodiment, the concentration of the pharmaceutically acceptable excipient is about 225 mM. In one embodiment, the concentration of the pharmaceutically acceptable excipient is about 220 mM. In one embodiment, the concentration of the pharmaceutically acceptable excipient is about 215 mM. In one embodiment, the concentration of the pharmaceutically acceptable excipient is about 210 mM. In one embodiment, the concentration of the pharmaceutically acceptable excipient is about 205 mM. In one embodiment, the concentration of the pharmaceutically acceptable excipient is about 200 mM. In one embodiment, the concentration of the pharmaceutically acceptable excipient is about 195 mM. In another embodiment, the concentration of the pharmaceutically acceptable excipient is about 190 mM. In one embodiment, the concentration of the pharmaceutically acceptable excipient is about 185 mM. In one embodiment, the concentration of the pharmaceutically acceptable excipient is about 180 mM. In one embodiment, the concentration of the pharmaceutically acceptable excipient is about 175 mM. In one embodiment, the concentration of the pharmaceutically acceptable excipient is about 170 mM. In one embodiment, the concentration of the pharmaceutically acceptable excipient is about 165 mM. In one embodiment, the concentration of the pharmaceutically acceptable excipient is about 160 mM. In one embodiment, the concentration of the pharmaceutically acceptable excipient is about 155 mM. In one embodiment, the concentration of the pharmaceutically acceptable excipient is about 150 mM. In one embodiment, the concentration of the pharmaceutically acceptable excipient is about 145 mM. In one embodiment, the concentration of the pharmaceutically acceptable excipient is about 140 mM. In one embodiment, the concentration of the pharmaceutically acceptable excipient is about 135 mM. In one embodiment, the concentration of the pharmaceutically acceptable excipient is about 130 mM. In one embodiment, the concentration of the pharmaceutically acceptable excipient is about 125 mM. In one embodiment, the concentration of the pharmaceutically acceptable excipient is about 120 mM. In one embodiment, the concentration of the pharmaceutically acceptable excipient is about 115 mM. In one embodiment, the concentration of the pharmaceutically acceptable excipient is about 110 mM. In one embodiment, the concentration of the pharmaceutically acceptable excipient is about 105 mM. In one embodiment, the concentration of the pharmaceutically acceptable excipient is about 100 mM. In one embodiment, the concentration of the pharmaceutically acceptable excipient is about 95 mM. In one embodiment, the concentration of the pharmaceutically acceptable excipient is about 90 mM. In one embodiment, the concentration of the pharmaceutically acceptable excipient is about 85 mM. In one embodiment, the concentration of the pharmaceutically acceptable excipient is about 80 mM. In one embodiment, the concentration of the pharmaceutically acceptable excipient is about 75 mM. In one embodiment, the concentration of the pharmaceutically acceptable excipient is about 70 mM. In one embodiment, the concentration of the pharmaceutically acceptable excipient is about 65 mM. In one embodiment, the concentration of the pharmaceutically acceptable excipient is about 60 mM. In one embodiment, the concentration of the pharmaceutically acceptable excipient is about 55 mM. In one embodiment, the concentration of the pharmaceutically acceptable excipient is about 50 mM. In one embodiment, the pharmaceutical composition having an anti-CD30L provided herein further comprises a pharmaceutically acceptable excipient, wherein the pharmaceutically acceptable excipient is arginine and the concentration of arginine is selected from those described in this paragraph for the pharmaceutically acceptable excipient. In one embodiment, the pharmaceutical composition having an anti-CD30L provided herein further comprises a pharmaceutically acceptable excipient, wherein the pharmaceutically acceptable excipient is arginine hydrochloride (arginine HCl) and the concentration of arginine HCl is selected from those described in this paragraph for the pharmaceutically acceptable excipient. In one embodiment, the pharmaceutical composition having an anti-CD30L provided herein further comprises a pharmaceutically acceptable excipient, wherein the pharmaceutically acceptable excipient is proline and the concentration of proline is selected from those described in this paragraph for the pharmaceutically acceptable excipient. In one embodiment, the pharmaceutical composition having an anti-CD30L provided herein further comprises a pharmaceutically acceptable excipient, wherein the pharmaceutically acceptable excipient is a polyol and the concentration of polyol is selected from those described in this paragraph for the pharmaceutically acceptable excipient. In one embodiment, the pharmaceutical composition having an anti-CD30L provided herein further comprises a pharmaceutically acceptable excipient, wherein the pharmaceutically acceptable excipient is sodium chloride and the concentration of sodium chloride is selected from those described in this paragraph for the pharmaceutically acceptable excipient. In one embodiment, the pharmaceutical composition having an anti-CD30L provided herein further comprises a pharmaceutically acceptable excipient, wherein the pharmaceutically acceptable excipient is glycine and the concentration of glycine is selected from those described in this paragraph for the pharmaceutically acceptable excipient. In one embodiment, the pharmaceutical composition having an anti-CD30L provided herein further comprises a pharmaceutically acceptable excipient, wherein the pharmaceutically acceptable excipient is lysine and the concentration of lysine is selected from those described in this paragraph for the pharmaceutically acceptable excipient. In one embodiment, the pharmaceutical composition having an anti-CD30L provided herein further comprises a pharmaceutically acceptable excipient, wherein the pharmaceutically acceptable excipient is lysine-hydrochloride and the concentration of lysine-hydrochloride is selected from those described in this paragraph for the pharmaceutically acceptable excipient. In one embodiment, the pharmaceutical composition having an anti-CD30L provided herein further comprises a pharmaceutically acceptable excipient, wherein the pharmaceutically acceptable excipient is arginine glutamate and the concentration of arginine glutamate is selected from those described in this paragraph for the pharmaceutically acceptable excipient. In one embodiment, the pharmaceutical composition having an anti-CD30L provided herein further comprises a pharmaceutically acceptable excipient, wherein the pharmaceutically acceptable excipient is potassium chloride and the concentration of potassium chloride is selected from those described in this paragraph for the pharmaceutically acceptable excipient. In one embodiment, the pharmaceutical composition having an anti-CD30L provided herein further comprises a pharmaceutically acceptable excipient, wherein the pharmaceutically acceptable excipient is magnesium chloride and the concentration of magnesium chloride is selected from those described in this paragraph for the pharmaceutically acceptable excipient. In one embodiment, the pharmaceutical composition having an anti-CD30L provided herein further comprises a pharmaceutically acceptable excipient, wherein the pharmaceutically acceptable excipient is calcium chloride and the concentration of calcium chloride is selected from those described in this paragraph for the pharmaceutically acceptable excipient.

Specifically, in one embodiment, the pharmaceutical composition having an anti-CD30L provided herein further comprises a pharmaceutically acceptable excipient, wherein the pharmaceutically acceptable excipient is arginine HCl. In one embodiment, the pharmaceutical composition having an anti-CD30L provided herein further comprises a pharmaceutically acceptable excipient, wherein the pharmaceutically acceptable excipient is arginine. In some embodiments, the pharmaceutical composition having an anti-CD30L provided herein contains about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, about 10%, about 12.5%, about 15%, about 17.5%, about 20%, about 25%, about 30% (w/v), or more arginine HCl (or arginine). In certain embodiments, the pharmaceutical composition of the antibodies provided herein contains 1-2.5%, 1.5-2.5%, 1-5%, 1-10%, 5-10%, 10-15%, 15-20%, 20-25%, 25-30%, 30-35%, 35-40%, (w/v), or more arginine HCl (or arginine). In one embodiment, the concentration of arginine HCl (or arginine) is from 1% to 2.5% (w/v). In one embodiment, the concentration of arginine HCl (or arginine) is from 1.5% to 2.5% (w/v). In one embodiment, the concentration of arginine HCl (or arginine) is from 1% to 5% (w/v). In one embodiment, the concentration of arginine HCl (or arginine) is from 1% to 10% (w/v). In one embodiment, the concentration of arginine HCl (or arginine) is about 1% (w/v). In one embodiment, the concentration of arginine HCl (or arginine) is about 1.1% (w/v). In one embodiment, the concentration of arginine HCl (or arginine) is about 1.2% (w/v). In one embodiment, the concentration of arginine HCl (or arginine) is about 1.3% (w/v). In one embodiment, the concentration of arginine HCl (or arginine) is about 1.4% (w/v). In one embodiment, the concentration of arginine HCl (or arginine) is about 1.5% (w/v). In one embodiment, the concentration of arginine HCl (or arginine) is about 1.6% (w/v). In one embodiment, the concentration of arginine HCl (or arginine) is about 1.7% (w/v). In one embodiment, the concentration of arginine HCl (or arginine) is about 1.8% (w/v). In one embodiment, the concentration of arginine HCl (or arginine) is about 1.9% (w/v). In one embodiment, the concentration of arginine HCl (or arginine) is about 2.0% (w/v). In one embodiment, the concentration of arginine HCl (or arginine) is about 2.1% (w/v). In one embodiment, the concentration of arginine HCl (or arginine) is about 2.2% (w/v). In one embodiment, the concentration of arginine HCl (or arginine) is about 2.3% (w/v). In one embodiment, the concentration of arginine HCl (or arginine) is about 2.4% (w/v). In one embodiment, the concentration of arginine HCl (or arginine) is about 2.5% (w/v). In one embodiment, the concentration of arginine HCl (or arginine) is about 2.6% (w/v). In one embodiment, the concentration of arginine HCl (or arginine) is about 2.7% (w/v). In one embodiment, the concentration of arginine HCl (or arginine) is about 2.8% (w/v). In one embodiment, the concentration of arginine HCl (or arginine) is about 2.9% (w/v). In one embodiment, the concentration of arginine HCl (or arginine) is about 3.0% (w/v). In one embodiment, the concentration of arginine HCl (or arginine) is about 4% (w/v). In one embodiment, the concentration of arginine HCl (or arginine) is about 5% (w/v). In one embodiment, the concentration of arginine HCl (or arginine) is about 6% (w/v). In one embodiment, the concentration of arginine HCl (or arginine) is about 7% (w/v). In one embodiment, the concentration of arginine HCl (or arginine) is about 8% (w/v). In one embodiment, the concentration of arginine HCl (or arginine) is about 9.0% (w/v). In some embodiments, the pharmaceutical composition of the antibodies provided herein contains about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 245 mM, about 250 mM, or more arginine HCl (or arginine). In certain embodiments, the pharmaceutical composition of the antibodies provided herein contains 20-150 mM, 20-140 mM, 20-130 mM, 20-120 mM, 20-110 mM, 20-100 mM, 20-90 mM, 20-80 mM, 20-70 mM, 20-60 mM, 20-50 mM, 30-150 mM, 30-140 mM, 30-130 mM, 30-120 mM, 30-110 mM, 30-100 mM, 30-90 mM, 30-80 mM, 30-70 mM, 30-60 mM, 30-50 mM, 40-150 mM, 40-140 mM, 40-130 mM, 40-120 mM, 40-110 mM, 40-100 mM, 40-90 mM, 40-80 mM, 40-70 mM, 40-60 mM, 40-50 mM, 50-150 mM, 50-140 mM, 50-130 mM, 50-120 mM, 50-110 mM, 50-100 mM, 50-90 mM, 50-80 mM, 50-70 mM, 50-60 mM, 100-150 mM, 100-300 mM, 150-300 mM, or more arginine HCl (or arginine). In one embodiment, the concentration of arginine HCl (or arginine) is from 50 to 150 mM. In one embodiment, the concentration of arginine HCl (or arginine) is from 60 to 140 mM. In one embodiment, the concentration of arginine HCl (or arginine) is from 70 to 130 mM. In one embodiment, the concentration of arginine HCl (or arginine) is from 80 to 120 mM. In one embodiment, the concentration of arginine HCl (or arginine) is from 90 to 110 mM. In one embodiment, the concentration of arginine HCl (or arginine) is from 50 to 300 mM. In one embodiment, the concentration of arginine HCl (or arginine) is from 100 to 300 mM. In one embodiment, the concentration of arginine HCl (or arginine) is about 250 mM. In one embodiment, the concentration of arginine HCl (or arginine) is about 245 mM. In one embodiment, the concentration of arginine HCl (or arginine) is about 240 mM. In one embodiment, the concentration of arginine HCl (or arginine) is about 235 mM. In one embodiment, the concentration of arginine HCl (or arginine) is about 230 mM. In one embodiment, the concentration of arginine HCl (or arginine) is about 225 mM. In one embodiment, the concentration of arginine HCl (or arginine) is about 220 mM. In one embodiment, the concentration of arginine HCl (or arginine) is about 215 mM. In one embodiment, the concentration of arginine HCl (or arginine) is about 210 mM. In one embodiment, the concentration of arginine HCl (or arginine) is about 205 mM. In one embodiment, the concentration of arginine HCl (or arginine) is about 200 mM. In one embodiment, the concentration of arginine HCl (or arginine) is about 195 mM. In another embodiment, the concentration of arginine HCl (or arginine) is about 190 mM. In one embodiment, the concentration of arginine HCl (or arginine) is about 185 mM. In one embodiment, the concentration of arginine HCl (or arginine) is about 180 mM. In one embodiment, the concentration of arginine HCl (or arginine) is about 175 mM. In one embodiment, the concentration of arginine HCl (or arginine) is about 170 mM. In one embodiment, the concentration of arginine HCl (or arginine) is about 165 mM. In one embodiment, the concentration of arginine HCl (or arginine) is about 160 mM. In one embodiment, the concentration of arginine HCl (or arginine) is about 155 mM. In one embodiment, the concentration of arginine HCl (or arginine) is about 150 mM. In one embodiment, the concentration of arginine HCl (or arginine) is about 145 mM. In one embodiment, the concentration of arginine HCl (or arginine) is about 140 mM. In one embodiment, the concentration of arginine HCl (or arginine) is about 135 mM. In one embodiment, the concentration of arginine HCl (or arginine) is about 130 mM. In one embodiment, the concentration of arginine HCl (or arginine) is about 125 mM. In one embodiment, the concentration of arginine HCl (or arginine) is about 120 mM. In one embodiment, the concentration of arginine HCl (or arginine) is about 115 mM. In one embodiment, the concentration of arginine HCl (or arginine) is about 110 mM. In one embodiment, the concentration of arginine HCl (or arginine) is about 105 mM. In one embodiment, the concentration of arginine HCl (or arginine) is about 100 mM. In one embodiment, the concentration of arginine HCl (or arginine) is about 95 mM. In one embodiment, the concentration of arginine HCl (or arginine) is about 90 mM. In one embodiment, the concentration of arginine HCl (or arginine) is about 85 mM. In one embodiment, the concentration of arginine HCl (or arginine) is about 80 mM. In one embodiment, the concentration of arginine HCl (or arginine) is about 75 mM. In one embodiment, the concentration of arginine HCl (or arginine) is about 70 mM. In one embodiment, the concentration of arginine HCl (or arginine) is about 65 mM. In one embodiment, the concentration of arginine HCl (or arginine) is about 60 mM. In one embodiment, the concentration of arginine HCl (or arginine) is about 55 mM. In one embodiment, the concentration of arginine HCl (or arginine) is about 50 mM.

Similarly, in various embodiments of the pharmaceutical composition provided herein, the pharmaceutical composition further comprises a pharmaceutically acceptable excipient, wherein the pharmaceutically acceptable excipient is a polyol. In various embodiments of the pharmaceutical composition provided herein, the pharmaceutically acceptable excipient is a polyol. In various embodiments of the pharmaceutical composition provided herein, the pharmaceutical composition further comprises a polyol. In some embodiments, the polyol is selected from the group consisting of sugar, sugar alcohol, and sugar acid. In one embodiment, the polyol is sugar. In another embodiment, the polyol is sugar alcohol. In yet another embodiment, the polyol is sugar acid. Non-limiting examples of polyol include sucrose, maltose, trehalose, mannitol, sorbitol, etc. In some embodiments, the pharmaceutical composition of the antibodies provided herein contains about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, about 10%, about 12.5%, about 15%, about 17.5%, about 20%, about 25%, about 30%, about 35%, about 40%, about 50% (w/v), or more sugar. In certain embodiments, the pharmaceutical composition of the antibodies provided herein contains 1-5%, 1-10%, 2.5-7.5%, 3-7%, 4-6%, 2.5-10%, 5-10%, 10-15%, 15-20%, 20-25%, 25-30%, 30-35%, 35-40% (w/v), or more sugar. In one embodiment, the concentration of the sugar is from 1% to 5% (w/v). In one embodiment, the concentration of the sugar is from 1% to 10% (w/v). In one embodiment, the concentration of the sugar is from 2.5% to 7.5% (w/v). In one embodiment, the concentration of the sugar is from 2.5% to 10% (w/v). In one embodiment, the concentration of the sugar is from 2% to 8% (w/v). In one embodiment, the concentration of the sugar is from 3% to 7% (w/v). In one embodiment, the concentration of the sugar is from 4% to 6% (w/v). In one embodiment, the concentration of the sugar is from 4.5% to 5.5% (w/v). In one embodiment, the concentration of the sugar is from 4.6% to 5.4% (w/v). In one embodiment, the concentration of the sugar is from 4.7% to 5.3% (w/v). In one embodiment, the concentration of the sugar is from 4.8% to 5.2% (w/v). In one embodiment, the concentration of the sugar is from 4.9% to 5.1% (w/v). In one embodiment, the concentration of the sugar is from 5% to 10% (w/v). In one embodiment, the concentration of the sugar is from 8.5% to 9.5% (w/v). In another embodiment, the concentration of the sugar is about 9.5% (w/v). In one embodiment, the concentration of the sugar is about 9.4% (w/v). In one embodiment, the concentration of the sugar is about 9.3% (w/v). In one embodiment, the concentration of the sugar is about 9.2% (w/v). In one embodiment, the concentration of the sugar is about 9.1% (w/v). In one embodiment, the concentration of the sugar is about 9.0% (w/v). In one embodiment, the concentration of the sugar is about 8.9% (w/v). In one embodiment, the concentration of the sugar is about 8.8% (w/v). In one embodiment, the concentration of the sugar is about 8.7% (w/v). In one embodiment, the concentration of the sugar is about 8.6% (w/v). In one embodiment, the concentration of the sugar is about 8.5% (w/v). In another embodiment, the concentration of the sugar is about 5.5% (w/v). In one embodiment, the concentration of the sugar is about 5.4% (w/v). In one embodiment, the concentration of the sugar is about 5.3% (w/v). In one embodiment, the concentration of the sugar is about 5.2% (w/v). In one embodiment, the concentration of the sugar is about 5.1% (w/v). In one embodiment, the concentration of the sugar is about 5.0% (w/v). In one embodiment, the concentration of the sugar is about 4.9% (w/v). In one embodiment, the concentration of the sugar is about 4.8% (w/v). In one embodiment, the concentration of the sugar is about 4.7% (w/v). In one embodiment, the concentration of the sugar is about 4.6% (w/v). In one embodiment, the concentration of the sugar is about 4.5% (w/v). In some embodiments, the pharmaceutical composition of the antibodies provided herein contains about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 245 mM, about 250 mM, about 255 mM, about 260 mM, about 265 mM, about 270 mM, about 275 mM, about 280 mM, about 285 mM, about 290 mM, about 320 mM, about 350 mM, about 380 mM, about 410 mM, about 440 mM, about 470 mM, about 500 mM, about 550 mM, about 600 mM, about 700 mM, about 800 mM, about 900 mM, about 1 M, or more sugar. In certain embodiments, the pharmaceutical composition of the antibodies provided herein contains 30-150 mM, 50-250 mM, 50-240 mM, 50-230 mM, 50-220 mM, 50-210 mM, 50-200 mM, 50-190 mM, 50-180 mM, 50-170 mM, 50-160 mM, 50-150 mM, 50-140 mM, 50-130 mM, 50-120 mM, 50-110 mM, 50-100 mM, 60-250 mM, 60-240 mM, 60-230 mM, 60-220 mM, 60-210 mM, 60-200 mM, 60-190 mM, 60-180 mM, 60-170 mM, 60-160 mM, 60-150 mM, 60-140 mM, 60-130 mM, 60-120 mM, 60-110 mM, 60-100 mM, 70-250 mM, 70-240 mM, 70-230 mM, 70-220 mM, 70-210 mM, 70-200 mM, 70-190 mM, 70-180 mM, 70-170 mM, 70-160 mM, 70-150 mM, 70-140 mM, 70-130 mM, 70-120 mM, 70-110 mM, 70-100 mM, 80-250 mM, 80-240 mM, 80-230 mM, 80-220 mM, 80-210 mM, 80-200 mM, 80-190 mM, 80-180 mM, 80-170 mM, 80-160 mM, 80-150 mM, 80-140 mM, 80-130 mM, 80-120 mM, 80-110 mM, 80-100 mM, 90-250 mM, 90-240 mM, 90-230 mM, 90-220 mM, 90-210 mM, 90-200 mM, 90-190 mM, 90-180 mM, 90-170 mM, 90-160 mM, 90-150 mM, 90-140 mM, 90-130 mM, 90-120 mM, 90-110 mM, 90-100 mM, 100-250 mM, 100-240 mM, 100-230 mM, 100-220 mM, 100-210 mM, 100-200 mM, 100-190 mM, 100-180 mM, 100-170 mM, 100-160 mM, 100-150 mM, 100-140 mM, 100-130 mM, 100-120 mM, 100-110 mM, 110-250 mM, 110-240 mM, 110-230 mM, 110-220 mM, 110-210 mM, 110-200 mM, 110-190 mM, 110-180 mM, 110-170 mM, 110-160 mM, 110-150 mM, 110-140 mM, 110-130 mM, 110-120 mM, 120-250 mM, 120-240 mM, 120-230 mM, 120-220 mM, 120-210 mM, 120-200 mM, 120-190 mM, 120-180 mM, 120-170 mM, 120-160 mM, 120-150 mM, 120-140 mM, 120-130 mM, 130-250 mM, 130-240 mM, 130-230 mM, 130-220 mM, 130-210 mM, 130-200 mM, 130-190 mM, 130-180 mM, 130-170 mM, 130-160 mM, 130-150 mM, 130-140 mM, 50-250 mM, 140-240 mM, 140-230 mM, 140-220 mM, 140-210 mM, 140-200 mM, 140-190 mM, 140-180 mM, 140-170 mM, 140-160 mM, 140-150 mM, 150-250 mM, 150-240 mM, 150-230 mM, 150-220 mM, 150-210 mM, 150-200 mM, 150-190 mM, 150-180 mM, 150-170 mM, 150-160 mM, 150-300 mM, 300-450 mM, 450-600 mM, 600-750 mM, 750-900 mM, 900 mM-1.05 M, 1.05-1.2 M, 1.2-1.35 M, 1.35-1.5M, or more sugar. In one embodiment, the concentration of the sugar is from 50 to 300 mM. In one embodiment, the concentration of the sugar is from 50 to 250 mM. In one embodiment, the concentration of the sugar is from 60 to 240 mM. In one embodiment, the concentration of the sugar is from 70 to 230 mM. In one embodiment, the concentration of the sugar is from 80 to 220 mM. In one embodiment, the concentration of the sugar is from 90 to 210 mM. In one embodiment, the concentration of the sugar is from 100 to 200 mM. In one embodiment, the concentration of the sugar is from 110 to 190 mM. In one embodiment, the concentration of the sugar is from 120 to 180 mM. In one embodiment, the concentration of the sugar is from 130 to 170 mM. In one embodiment, the concentration of the sugar is from 140 to 160 mM. In one embodiment, the concentration of the sugar is from 245 mM to 275 mM. In another embodiment, the concentration of the sugar is about 290 mM. In one embodiment, the concentration of the sugar is about 285 mM. In one embodiment, the concentration of the sugar is about 280 mM. In one embodiment, the concentration of the sugar is about 275 mM. In one embodiment, the concentration of the sugar is about 270 mM. In one embodiment, the concentration of the sugar is about 265 mM. In one embodiment, the concentration of the sugar is about 260 mM. In one embodiment, the concentration of the sugar is about 255 mM. In one embodiment, the concentration of the sugar is about 250 mM. In one embodiment, the concentration of the sugar is about 245 mM. In one embodiment, the concentration of the sugar is about 240 mM. In another embodiment, the concentration of the sugar is 290 mM. In one embodiment, the concentration of the sugar is 285 mM. In one embodiment, the concentration of the sugar is 280 mM. In one embodiment, the concentration of the sugar is 275 mM. In one embodiment, the concentration of the sugar is 270 mM. In one embodiment, the concentration of the sugar is 265 mM. In one embodiment, the concentration of the sugar is 260 mM. In one embodiment, the concentration of the sugar is 255 mM. In one embodiment, the concentration of the sugar is 250 mM. In one embodiment, the concentration of the sugar is 245 mM. In one embodiment, the concentration of the sugar is 240 mM. In one embodiment, the concentration of the sugar is about 235 mM. In one embodiment, the concentration of the sugar is about 230 mM. In one embodiment, the concentration of the sugar is about 225 mM. In one embodiment, the concentration of the sugar is about 220 mM. In one embodiment, the concentration of the sugar is about 215 mM. In one embodiment, the concentration of the sugar is about 210 mM. In one embodiment, the concentration of the sugar is about 205 mM. In one embodiment, the concentration of the sugar is about 200 mM. In one embodiment, the concentration of the sugar is about 195 mM. In another embodiment, the concentration of the sugar is about 190 mM. In one embodiment, the concentration of the sugar is about 185 mM. In one embodiment, the concentration of the sugar is about 180 mM. In one embodiment, the concentration of the sugar is about 175 mM. In one embodiment, the concentration of the sugar is about 170 mM. In one embodiment, the concentration of the sugar is about 165 mM. In one embodiment, the concentration of the sugar is about 160 mM. In one embodiment, the concentration of the sugar is about 155 mM. In one embodiment, the concentration of the sugar is about 150 mM. In one embodiment, the concentration of the sugar is about 145 mM. In one embodiment, the concentration of the sugar is about 140 mM. In one embodiment, the concentration of the sugar is about 135 mM. In one embodiment, the concentration of the sugar is about 130 mM. In one embodiment, the concentration of the sugar is about 125 mM. In one embodiment, the concentration of the sugar is about 120 mM. In one embodiment, the concentration of the sugar is about 115 mM. In one embodiment, the concentration of the sugar is about 110 mM. In one embodiment, the concentration of the sugar is about 105 mM. In one embodiment, the concentration of the sugar is about 100 mM. In one embodiment, the concentration of the sugar is about 95 mM. In one embodiment, the concentration of the sugar is about 90 mM. In one embodiment, the concentration of the sugar is about 85 mM. In one embodiment, the concentration of the sugar is about 80 mM. In one embodiment, the concentration of the sugar is about 75 mM. In one embodiment, the concentration of the sugar is about 70 mM. In one embodiment, the concentration of the sugar is about 65 mM. In one embodiment, the concentration of the sugar is about 60 mM. In one embodiment, the concentration of the sugar is about 55 mM. In one embodiment, the concentration of the sugar is about 50 mM. In some embodiments of the pharmaceutical composition provided herein including in this paragraph that comprises a sugar, the sugar in the pharmaceutical composition is sucrose. In certain embodiments of the pharmaceutical composition provided herein including in this paragraph that comprises a sugar, the sugar in the pharmaceutical composition is maltose. In some embodiments of the pharmaceutical composition provided herein including in this paragraph that comprises a sugar, the sugar in the pharmaceutical composition is trehalose. In certain embodiments of the pharmaceutical composition provided herein including in this paragraph that comprises a sugar, the sugar in the pharmaceutical composition is mannitol. In certain embodiments of the pharmaceutical composition provided herein including in this paragraph that comprises a sugar, the sugar in the pharmaceutical composition is sorbitol.

### 4.5.4 Surfactant in the Pharmaceutical Composition Comprising Anti-CD30L Antibody or Antigen Binding Fragment

Certain embodiments of the surfactant in the pharmaceutical compositions comprising anti-CD30L provided in Section 4.5 are described and provided in this Section 4.5.4. Accordingly, in certain embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition comprises a buffer provided in Section 4.5.2, wherein the pharmaceutical composition comprises a pharmaceutically acceptable excipient provided in Section 4.5.3, and wherein the pharmaceutical composition comprises a surfactant provided in this Section 4.5.4. In some embodiments, provided herein is a pharmaceutical composition comprising an anti-CD30L antibody or antigen binding fragment thereof provided in Sections 2 and 4.1, wherein the pharmaceutical composition comprises a surfactant provided in this Section 4.5.4. In certain embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition comprises a surfactant provided in this Section 4.5.4. In certain embodiments, provided herein is a pharmaceutical composition comprising an anti-CD30L antibody or antigen binding fragment thereof provided in Sections 2 and 4.1, wherein the pharmaceutical composition comprises a buffer provided in Section 4.5.2, and wherein the pharmaceutical composition comprises a surfactant provided in this Section 4.5.4. In some embodiments, provided herein is a pharmaceutical composition comprising an anti-CD30L antibody or antigen binding fragment thereof provided in Sections 2 and 4.1, wherein the pharmaceutical composition comprises a pharmaceutically acceptable excipient provided in Section 4.5.3, and wherein the pharmaceutical composition comprises a surfactant provided in this Section 4.5.4. In some embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition comprises a buffer provided in Section 4.5.2, and wherein the pharmaceutical composition comprises a surfactant provided in this Section 4.5.4. In some embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition comprises a pharmaceutically acceptable excipient provided in Section 4.5.3, and wherein the pharmaceutical composition comprises a surfactant provided in this Section 4.5.4. In some embodiments, provided herein is a pharmaceutical composition comprising an anti-CD30L antibody or antigen binding fragment thereof provided in Sections 2 and 4.1, wherein the pharmaceutical composition comprises a buffer provided in Section 4.5.2, wherein the pharmaceutical composition comprises a pharmaceutically acceptable excipient provided in Section 4.5.3, and wherein the pharmaceutical composition comprises a surfactant provided in this Section 4.5.4.

In various embodiments of the pharmaceutical compositions provided herein, the pharmaceutical compositions further comprises a surfactant, including but not limited to polysorbate 20, polysorbate 40, polysorbate 60, and polysorbate 80. In one embodiment, the surfactant is a polysorbate. In one embodiment, the surfactant is polysorbate 20. In another embodiment, the surfactant is polysorbate 40. In one embodiment, the surfactant is polysorbate 60. In another embodiment, the surfactant is polysorbate 80. In one embodiment, the surfactant is poloxamer 188 (P188). In some embodiments, the pharmaceutical composition of the anti-CD30L provided herein contains 0.001%, 0.002%, 0.003%, 0.004%, 0.005%, 0.006%, 0.007%, 0.008%, 0.009%, 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, 0.03%, 0.035%, 0.04%, 0.045%, 0.05%, 0.055%, 0.06%, 0.065%, 0.07%, 0.075%, 0.08%, 0.085%, 0.09%, 0.095%, 0.1%, 0.15%, 0.2%, 0.3%, 0.4%, 0.5% (w/v), or more polysorbate. In other embodiments, the pharmaceutical composition of the anti-CD30L provided herein contains 0.001-0.005%, 0.005-0.01%, 0.005-0.03%, 0.005-0.025%, 0.005-0.02%, 0.005-0.015%, 0.01-0.03%, 0.01-0.025%, 0.01-0.02%, 0.015-0.03%, 0.015-0.025%, 0.015-0.02%, 0.01-0.04%, 0.01-0.05%, 0.05-0.1%, 0.1-0.5% (w/v), or more polysorbate. In one embodiment, the pharmaceutical composition of the anti-CD30L provided herein contains about 0.005% (w/v) polysorbate. In one embodiment, the pharmaceutical composition of the anti-CD30L provided herein contains about 0.01% (w/v) polysorbate. In one embodiment, the pharmaceutical composition of the anti-CD30L provided herein contains about 0.015% (w/v) polysorbate. In one embodiment, the pharmaceutical composition of the anti-CD30L provided herein contains about 0.02% (w/v) polysorbate. In one embodiment, the pharmaceutical composition of the anti-CD30L provided herein contains about 0.025% (w/v) polysorbate. In one embodiment, the pharmaceutical composition of the anti-CD30L provided herein contains about 0.03% (w/v) polysorbate. In one embodiment, the pharmaceutical composition of the anti-CD30L provided herein contains about 0.035% (w/v) polysorbate. In one embodiment, the pharmaceutical composition of the anti-CD30L provided herein contains about 0.04% (w/v) polysorbate. In one embodiment, the pharmaceutical composition of the anti-CD30L provided herein contains about 0.045% (w/v) polysorbate. In one embodiment, the pharmaceutical composition of the anti-CD30L provided herein contains about 0.05% (w/v) polysorbate. In one embodiment, the pharmaceutical composition of the anti-CD30L provided herein contains 0.001 to 0.05% (w/v) polysorbate. In one embodiment, the pharmaceutical composition of the anti-CD30L provided herein contains 0.001-0.005% (w/v) polysorbate. In one embodiment, the pharmaceutical composition of the anti-CD30L provided herein contains 0.005-0.01% (w/v) polysorbate. In one embodiment, the pharmaceutical composition of the anti-CD30L provided herein contains 0.005-0.03%, (w/v) polysorbate. In one embodiment, the pharmaceutical composition of the anti-CD30L provided herein contains 0.005-0.025%, (w/v) polysorbate. In one embodiment, the pharmaceutical composition of the anti-CD30L provided herein contains 0.005-0.02%, (w/v) polysorbate. In one embodiment, the pharmaceutical composition of the anti-CD30L provided herein contains 0.01-0.03% (w/v) polysorbate. In one embodiment, the pharmaceutical composition of the anti-CD30L provided herein contains 0.01-0.025% (w/v) polysorbate. In one embodiment, the pharmaceutical composition of the anti-CD30L provided herein contains 0.01-0.02% (w/v) polysorbate. In one embodiment, the pharmaceutical composition of the anti-CD30L provided herein contains 0.015-0.03% (w/v) polysorbate. In one embodiment, the pharmaceutical composition of the anti-CD30L provided herein contains 0.015-0.025% (w/v) polysorbate. In one embodiment, the pharmaceutical composition of the anti-CD30L provided herein contains 0.015-0.02% (w/v) polysorbate. In one embodiment, the pharmaceutical composition of the anti-CD30L provided herein contains 0.01-0.04% (w/v) polysorbate. In one embodiment, the pharmaceutical composition of the anti-CD30L provided herein contains 0.01-0.05% (w/v) polysorbate. In one embodiment, the pharmaceutical composition of the anti-CD30L provided herein contains 0.005-0.05% (w/v) polysorbate. The disclosure provides that the polysorbate described in various embodiments in this paragraph can be polysorbate 20, polysorbate 40, polysorbate 60, or polysorbate 80.

In various embodiments of the pharmaceutical compositions provided herein, the pharmaceutical compositions further comprises polysorbate 20. In some embodiments, the pharmaceutical composition of the anti-CD30L provided herein contains 0.001%, 0.002%, 0.003%, 0.004%, 0.005%, 0.006%, 0.007%, 0.008%, 0.009%, 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, 0.03%, 0.035%, 0.04%, 0.045%, 0.05%, 0.055%, 0.06%, 0.065%, 0.07%, 0.075%, 0.08%, 0.085%, 0.09%, 0.095%, 0.1%, 0.15%, 0.2%, 0.3%, 0.4%, 0.5% (w/v), or more polysorbate 20. In other embodiments, the pharmaceutical composition of the anti-CD30L provided herein contains 0.001-0.005%, 0.005-0.01%, 0.005-0.03%, 0.005-0.025%, 0.005-0.02%, 0.005-0.015%, 0.01-0.03%, 0.01-0.025%, 0.01-0.02%, 0.015-0.03%, 0.015-0.025%, 0.015-0.02%, 0.01-0.04%, 0.01-0.05%, 0.05-0.1%, 0.1-0.5% (w/v), or more polysorbate 20. In one embodiment, the pharmaceutical composition of the anti-CD30L provided herein contains about 0.005% (w/v) polysorbate 20. In one embodiment, the pharmaceutical composition of the anti-CD30L provided herein contains about 0.01% (w/v) polysorbate 20. In one embodiment, the pharmaceutical composition of the anti-CD30L provided herein contains about 0.015% (w/v) polysorbate 20. In one embodiment, the pharmaceutical composition of the anti-CD30L provided herein contains about 0.02% (w/v) polysorbate 20. In one embodiment, the pharmaceutical composition of the anti-CD30L provided herein contains about 0.025% (w/v) polysorbate 20. In one embodiment, the pharmaceutical composition of the anti-CD30L provided herein contains about 0.03% (w/v) polysorbate 20. In one embodiment, the pharmaceutical composition of the anti-CD30L provided herein contains about 0.035% (w/v) polysorbate 20. In one embodiment, the pharmaceutical composition of the anti-CD30L provided herein contains about 0.04% (w/v) polysorbate 20. In one embodiment, the pharmaceutical composition of the anti-CD30L provided herein contains about 0.045% (w/v) polysorbate 20. In one embodiment, the pharmaceutical composition of the anti-CD30L provided herein contains about 0.05% (w/v) polysorbate 20. In one embodiment, the pharmaceutical composition of the anti-CD30L provided herein contains 0.001 to 0.05% (w/v) polysorbate 20. In one embodiment, the pharmaceutical composition of the anti-CD30L provided herein contains 0.001-0.005% (w/v) polysorbate 20. In one embodiment, the pharmaceutical composition of the anti-CD30L provided herein contains 0.005-0.01% (w/v) polysorbate 20. In one embodiment, the pharmaceutical composition of the anti-CD30L provided herein contains 0.005-0.03%, (w/v) polysorbate 20. In one embodiment, the pharmaceutical composition of the anti-CD30L provided herein contains 0.005-0.025%, (w/v) polysorbate 20. In one embodiment, the pharmaceutical composition of the anti-CD30L provided herein contains 0.005-0.02%, (w/v) polysorbate 20. In one embodiment, the pharmaceutical composition of the anti-CD30L provided herein contains 0.01-0.03% (w/v) polysorbate 20. In one embodiment, the pharmaceutical composition of the anti-CD30L provided herein contains 0.01-0.025% (w/v) polysorbate 20. In one embodiment, the pharmaceutical composition of the anti-CD30L provided herein contains 0.01-0.02% (w/v) polysorbate 20. In one embodiment, the pharmaceutical composition of the anti-CD30L provided herein contains 0.015-0.03% (w/v) polysorbate 20. In one embodiment, the pharmaceutical composition of the anti-CD30L provided herein contains 0.015-0.025% (w/v) polysorbate 20. In one embodiment, the pharmaceutical composition of the anti-CD30L provided herein contains 0.015-0.02% (w/v) polysorbate 20. In one embodiment, the pharmaceutical composition of the anti-CD30L provided herein contains 0.01-0.04% (w/v) polysorbate 20. In one embodiment, the pharmaceutical composition of the anti-CD30L provided herein contains 0.01-0.05% (w/v) polysorbate 20. In one embodiment, the pharmaceutical composition of the anti-CD30L provided herein contains 0.005-0.05% (w/v) polysorbate 20.

In various embodiments of the pharmaceutical compositions provided herein, the pharmaceutical compositions further comprises polysorbate 40. In some embodiments, the pharmaceutical composition of the anti-CD30L provided herein contains 0.001%, 0.002%, 0.003%, 0.004%, 0.005%, 0.006%, 0.007%, 0.008%, 0.009%, 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, 0.03%, 0.035%, 0.04%, 0.045%, 0.05%, 0.055%, 0.06%, 0.065%, 0.07%, 0.075%, 0.08%, 0.085%, 0.09%, 0.095%, 0.1%, 0.15%, 0.2%, 0.3%, 0.4%, 0.5% (w/v), or more polysorbate 40. In other embodiments, the pharmaceutical composition of the anti-CD30L provided herein contains 0.001-0.005%, 0.005-0.01%, 0.005-0.03%, 0.005-0.025%, 0.005-0.02%, 0.005-0.015%, 0.01-0.03%, 0.01-0.025%, 0.01-0.02%, 0.015-0.03%, 0.015-0.025%, 0.015-0.02%, 0.01-0.04%, 0.01-0.05%, 0.05-0.1%, 0.1-0.5% (w/v), or more polysorbate 40. In one embodiment, the pharmaceutical composition of the anti-CD30L provided herein contains about 0.005% (w/v) polysorbate 40. In one embodiment, the pharmaceutical composition of the anti-CD30L provided herein contains about 0.01% (w/v) polysorbate 40. In one embodiment, the pharmaceutical composition of the anti-CD30L provided herein contains about 0.015% (w/v) polysorbate 40. In one embodiment, the pharmaceutical composition of the anti-CD30L provided herein contains about 0.02% (w/v) polysorbate 40. In one embodiment, the pharmaceutical composition of the anti-CD30L provided herein contains about 0.025% (w/v) polysorbate 40. In one embodiment, the pharmaceutical composition of the anti-CD30L provided herein contains about 0.03% (w/v) polysorbate 40. In one embodiment, the pharmaceutical composition of the anti-CD30L provided herein contains about 0.035% (w/v) polysorbate 40. In one embodiment, the pharmaceutical composition of the anti-CD30L provided herein contains about 0.04% (w/v) polysorbate 40. In one embodiment, the pharmaceutical composition of the anti-CD30L provided herein contains about 0.045% (w/v) polysorbate 40. In one embodiment, the pharmaceutical composition of the anti-CD30L provided herein contains about 0.05% (w/v) polysorbate 40. In one embodiment, the pharmaceutical composition of the anti-CD30L provided herein contains 0.001 to 0.05% (w/v) polysorbate 40. In one embodiment, the pharmaceutical composition of the anti-CD30L provided herein contains 0.001-0.005% (w/v) polysorbate 40. In one embodiment, the pharmaceutical composition of the anti-CD30L provided herein contains 0.005-0.01% (w/v) polysorbate 40. In one embodiment, the pharmaceutical composition of the anti-CD30L provided herein contains 0.005-0.03%, (w/v) polysorbate 40. In one embodiment, the pharmaceutical composition of the anti-CD30L provided herein contains 0.005-0.025%, (w/v) polysorbate 40. In one embodiment, the pharmaceutical composition of the anti-CD30L provided herein contains 0.005-0.02%, (w/v) polysorbate 40. In one embodiment, the pharmaceutical composition of the anti-CD30L provided herein contains 0.01-0.03% (w/v) polysorbate 40. In one embodiment, the pharmaceutical composition of the anti-CD30L provided herein contains 0.01-0.025% (w/v) polysorbate 40. In one embodiment, the pharmaceutical composition of the anti-CD30L provided herein contains 0.01-0.02% (w/v) polysorbate 40. In one embodiment, the pharmaceutical composition of the anti-CD30L provided herein contains 0.015-0.03% (w/v) polysorbate 40. In one embodiment, the pharmaceutical composition of the anti-CD30L provided herein contains 0.015-0.025% (w/v) polysorbate 40. In one embodiment, the pharmaceutical composition of the anti-CD30L provided herein contains 0.015-0.02% (w/v) polysorbate 40. In one embodiment, the pharmaceutical composition of the anti-CD30L provided herein contains 0.01-0.04% (w/v) polysorbate 40. In one embodiment, the pharmaceutical composition of the anti-CD30L provided herein contains 0.01-0.05% (w/v) polysorbate 40. In one embodiment, the pharmaceutical composition of the anti-CD30L provided herein contains 0.005-0.05% (w/v) polysorbate 40.

In various embodiments of the pharmaceutical compositions provided herein, the pharmaceutical compositions further comprises polysorbate 60. In some embodiments, the pharmaceutical composition of the anti-CD30L provided herein contains 0.001%, 0.002%, 0.003%, 0.004%, 0.005%, 0.006%, 0.007%, 0.008%, 0.009%, 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, 0.03%, 0.035%, 0.04%, 0.045%, 0.05%, 0.055%, 0.06%, 0.065%, 0.07%, 0.075%, 0.08%, 0.085%, 0.09%, 0.095%, 0.1%, 0.15%, 0.2%, 0.3%, 0.4%, 0.5% (w/v), or more polysorbate 60. In other embodiments, the pharmaceutical composition of the anti-CD30L provided herein contains 0.001-0.005%, 0.005-0.01%, 0.005-0.03%, 0.005-0.025%, 0.005-0.02%, 0.005-0.015%, 0.01-0.03%, 0.01-0.025%, 0.01-0.02%, 0.015-0.03%, 0.015-0.025%, 0.015-0.02%, 0.01-0.04%, 0.01-0.05%, 0.05-0.1%, 0.1-0.5% (w/v), or more polysorbate 60. In one embodiment, the pharmaceutical composition of the anti-CD30L provided herein contains about 0.005% (w/v) polysorbate 60. In one embodiment, the pharmaceutical composition of the anti-CD30L provided herein contains about 0.01% (w/v) polysorbate 60. In one embodiment, the pharmaceutical composition of the anti-CD30L provided herein contains about 0.015% (w/v) polysorbate 60. In one embodiment, the pharmaceutical composition of the anti-CD30L provided herein contains about 0.02% (w/v) polysorbate 60. In one embodiment, the pharmaceutical composition of the anti-CD30L provided herein contains about 0.025% (w/v) polysorbate 60. In one embodiment, the pharmaceutical composition of the anti-CD30L provided herein contains about 0.03% (w/v) polysorbate 60. In one embodiment, the pharmaceutical composition of the anti-CD30L provided herein contains about 0.035% (w/v) polysorbate 60. In one embodiment, the pharmaceutical composition of the anti-CD30L provided herein contains about 0.04% (w/v) polysorbate 60. In one embodiment, the pharmaceutical composition of the anti-CD30L provided herein contains about 0.045% (w/v) polysorbate 60. In one embodiment, the pharmaceutical composition of the anti-CD30L provided herein contains about 0.05% (w/v) polysorbate 60. In one embodiment, the pharmaceutical composition of the anti-CD30L provided herein contains 0.001 to 0.05% (w/v) polysorbate 60. In one embodiment, the pharmaceutical composition of the anti-CD30L provided herein contains 0.001-0.005% (w/v) polysorbate 60. In one embodiment, the pharmaceutical composition of the anti-CD30L provided herein contains 0.005-0.01% (w/v) polysorbate 60. In one embodiment, the pharmaceutical composition of the anti-CD30L provided herein contains 0.005-0.03%, (w/v) polysorbate 60. In one embodiment, the pharmaceutical composition of the anti-CD30L provided herein contains 0.005-0.025%, (w/v) polysorbate 60. In one embodiment, the pharmaceutical composition of the anti-CD30L provided herein contains 0.005-0.02%, (w/v) polysorbate 60. In one embodiment, the pharmaceutical composition of the anti-CD30L provided herein contains 0.01-0.03% (w/v) polysorbate 60. In one embodiment, the pharmaceutical composition of the anti-CD30L provided herein contains 0.01-0.025% (w/v) polysorbate 60. In one embodiment, the pharmaceutical composition of the anti-CD30L provided herein contains 0.01-0.02% (w/v) polysorbate 60. In one embodiment, the pharmaceutical composition of the anti-CD30L provided herein contains 0.015-0.03% (w/v) polysorbate 60. In one embodiment, the pharmaceutical composition of the anti-CD30L provided herein contains 0.015-0.025% (w/v) polysorbate 60. In one embodiment, the pharmaceutical composition of the anti-CD30L provided herein contains 0.015-0.02% (w/v) polysorbate 60. In one embodiment, the pharmaceutical composition of the anti-CD30L provided herein contains 0.01-0.04% (w/v) polysorbate 60. In one embodiment, the pharmaceutical composition of the anti-CD30L provided herein contains 0.01-0.05% (w/v) polysorbate 60. In one embodiment, the pharmaceutical composition of the anti-CD30L provided herein contains 0.005-0.05% (w/v) polysorbate 60.

In various embodiments of the pharmaceutical compositions provided herein, the pharmaceutical compositions further comprises polysorbate 80. In some embodiments, the pharmaceutical composition of the anti-CD30L provided herein contains 0.001%, 0.002%, 0.003%, 0.004%, 0.005%, 0.006%, 0.007%, 0.008%, 0.009%, 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, 0.03%, 0.035%, 0.04%, 0.045%, 0.05%, 0.055%, 0.06%, 0.065%, 0.07%, 0.075%, 0.08%, 0.085%, 0.09%, 0.095%, 0.1%, 0.15%, 0.2%, 0.3%, 0.4%, 0.5% (w/v), or more polysorbate 80. In other embodiments, the pharmaceutical composition of the anti-CD30L provided herein contains 0.001-0.005%, 0.005-0.01%, 0.005-0.03%, 0.005-0.025%, 0.005-0.02%, 0.005-0.015%, 0.01-0.03%, 0.01-0.025%, 0.01-0.02%, 0.015-0.03%, 0.015-0.025%, 0.015-0.02%, 0.01-0.04%, 0.01-0.05%, 0.05-0.1%, 0.1-0.5% (w/v), or more polysorbate 80. In one embodiment, the pharmaceutical composition of the anti-CD30L provided herein contains about 0.005% (w/v) polysorbate 80. In one embodiment, the pharmaceutical composition of the anti-CD30L provided herein contains about 0.01% (w/v) polysorbate 80. In one embodiment, the pharmaceutical composition of the anti-CD30L provided herein contains about 0.015% (w/v) polysorbate 80. In one embodiment, the pharmaceutical composition of the anti-CD30L provided herein contains about 0.02% (w/v) polysorbate 80. In one embodiment, the pharmaceutical composition of the anti-CD30L provided herein contains about 0.025% (w/v) polysorbate 80. In one embodiment, the pharmaceutical composition of the anti-CD30L provided herein contains about 0.03% (w/v) polysorbate 80. In one embodiment, the pharmaceutical composition of the anti-CD30L provided herein contains about 0.035% (w/v) polysorbate 80. In one embodiment, the pharmaceutical composition of the anti-CD30L provided herein contains about 0.04% (w/v) polysorbate 80. In one embodiment, the pharmaceutical composition of the anti-CD30L provided herein contains about 0.045% (w/v) polysorbate 80. In one embodiment, the pharmaceutical composition of the anti-CD30L provided herein contains about 0.05% (w/v) polysorbate 80. In one embodiment, the pharmaceutical composition of the anti-CD30L provided herein contains 0.001 to 0.05% (w/v) polysorbate 80. In one embodiment, the pharmaceutical composition of the anti-CD30L provided herein contains 0.001-0.005% (w/v) polysorbate 80. In one embodiment, the pharmaceutical composition of the anti-CD30L provided herein contains 0.005-0.01% (w/v) polysorbate 80. In one embodiment, the pharmaceutical composition of the anti-CD30L provided herein contains 0.005-0.03%, (w/v) polysorbate 80. In one embodiment, the pharmaceutical composition of the anti-CD30L provided herein contains 0.005-0.025%, (w/v) polysorbate 80. In one embodiment, the pharmaceutical composition of the anti-CD30L provided herein contains 0.005-0.02%, (w/v) polysorbate 80. In one embodiment, the pharmaceutical composition of the anti-CD30L provided herein contains 0.01-0.03% (w/v) polysorbate 80. In one embodiment, the pharmaceutical composition of the anti-CD30L provided herein contains 0.01-0.025% (w/v) polysorbate 80. In one embodiment, the pharmaceutical composition of the anti-CD30L provided herein contains 0.01-0.02% (w/v) polysorbate 80. In one embodiment, the pharmaceutical composition of the anti-CD30L provided herein contains 0.015-0.03% (w/v) polysorbate 80. In one embodiment, the pharmaceutical composition of the anti-CD30L provided herein contains 0.015-0.025% (w/v) polysorbate 80. In one embodiment, the pharmaceutical composition of the anti-CD30L provided herein contains 0.015-0.02% (w/v) polysorbate 80. In one embodiment, the pharmaceutical composition of the anti-CD30L provided herein contains 0.01-0.04% (w/v) polysorbate 80. In one embodiment, the pharmaceutical composition of the anti-CD30L provided herein contains 0.01-0.05% (w/v) polysorbate 80. In one embodiment, the pharmaceutical composition of the anti-CD30L provided herein contains 0.005-0.05% (w/v) polysorbate 80.

In various embodiments of the pharmaceutical compositions provided herein, the pharmaceutical compositions further comprises poloxamer 188. In some embodiments, the pharmaceutical composition of the anti-CD30L provided herein contains 0.001%, 0.002%, 0.003%, 0.004%, 0.005%, 0.006%, 0.007%, 0.008%, 0.009%, 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, 0.03%, 0.035%, 0.04%, 0.045%, 0.05%, 0.055%, 0.06%, 0.065%, 0.07%, 0.075%, 0.08%, 0.085%, 0.09%, 0.095%, 0.1%, 0.15%, 0.2%, 0.3%, 0.4%, 0.5% (w/v), or more poloxamer 188. In other embodiments, the pharmaceutical composition of the anti-CD30L provided herein contains 0.001-0.005%, 0.005-0.01%, 0.005-0.03%, 0.005-0.025%, 0.005-0.02%, 0.005-0.015%, 0.01-0.03%, 0.01-0.025%, 0.01-0.02%, 0.015-0.03%, 0.015-0.025%, 0.015-0.02%, 0.01-0.04%, 0.01-0.05%, 0.05-0.1%, 0.1-0.5% (w/v), or more poloxamer 188. In one embodiment, the pharmaceutical composition of the anti-CD30L provided herein contains about 0.005% (w/v) poloxamer 188. In one embodiment, the pharmaceutical composition of the anti-CD30L provided herein contains about 0.01% (w/v) poloxamer 188. In one embodiment, the pharmaceutical composition of the anti-CD30L provided herein contains about 0.015% (w/v) poloxamer 188. In one embodiment, the pharmaceutical composition of the anti-CD30L provided herein contains about 0.02% (w/v) poloxamer 188. In one embodiment, the pharmaceutical composition of the anti-CD30L provided herein contains about 0.025% (w/v) poloxamer 188. In one embodiment, the pharmaceutical composition of the anti-CD30L provided herein contains about 0.03% (w/v) poloxamer 188. In one embodiment, the pharmaceutical composition of the anti-CD30L provided herein contains about 0.035% (w/v) poloxamer 188. In one embodiment, the pharmaceutical composition of the anti-CD30L provided herein contains about 0.04% (w/v) poloxamer 188. In one embodiment, the pharmaceutical composition of the anti-CD30L provided herein contains about 0.045% (w/v) poloxamer 188. In one embodiment, the pharmaceutical composition of the anti-CD30L provided herein contains about 0.05% (w/v) poloxamer 188. In one embodiment, the pharmaceutical composition of the anti-CD30L provided herein contains 0.001 to 0.05% (w/v) poloxamer 188. In one embodiment, the pharmaceutical composition of the anti-CD30L provided herein contains 0.001-0.005% (w/v) poloxamer 188. In one embodiment, the pharmaceutical composition of the anti-CD30L provided herein contains 0.005-0.01% (w/v) poloxamer 188. In one embodiment, the pharmaceutical composition of the anti-CD30L provided herein contains 0.005-0.03%, (w/v) poloxamer 188. In one embodiment, the pharmaceutical composition of the anti-CD30L provided herein contains 0.005-0.025% (w/v) poloxamer 188. In one embodiment, the pharmaceutical composition of the anti-CD30L provided herein contains 0.005-0.02% (w/v) poloxamer 188. In one embodiment, the pharmaceutical composition of the anti-CD30L provided herein contains 0.01-0.03% (w/v) poloxamer 188. In one embodiment, the pharmaceutical composition of the anti-CD30L provided herein contains 0.01-0.025% (w/v) poloxamer 188. In one embodiment, the pharmaceutical composition of the anti-CD30L provided herein contains 0.01-0.02% (w/v) poloxamer 188. In one embodiment, the pharmaceutical composition of the anti-CD30L provided herein contains 0.015-0.03% (w/v) poloxamer 188. In one embodiment, the pharmaceutical composition of the anti-CD30L provided herein contains 0.015-0.025% (w/v) poloxamer 188. In one embodiment, the pharmaceutical composition of the anti-CD30L provided herein contains 0.015-0.02% (w/v) poloxamer 188. In one embodiment, the pharmaceutical composition of the anti-CD30L provided herein contains 0.01-0.04% (w/v) poloxamer 188. In one embodiment, the pharmaceutical composition of the anti-CD30L provided herein contains 0.01-0.05% (w/v) poloxamer 188. In one embodiment, the pharmaceutical composition of the anti-CD30L provided herein contains 0.005-0.05% (w/v) poloxamer 188.

In one embodiment, the concentration of the surfactant in the pharmaceutical composition is from 0.001% to 0.1% (w/v). In one embodiment, the concentration of the surfactant in the pharmaceutical composition is from 0.001% to 0.05% (w/v). In one embodiment, the concentration of the surfactant in the pharmaceutical composition is from 0.001% to 0.02% (w/v). In one embodiment, the concentration of the surfactant in the pharmaceutical composition is from 0.001% to 0.01% (w/v). In one embodiment, the concentration of the surfactant in the pharmaceutical composition is from 0.001% to 0.005% (w/v). In one embodiment, the concentration of the surfactant in the pharmaceutical composition is from 0.005% to 0.01% (w/v). In one embodiment, the concentration of the surfactant in the pharmaceutical composition is from 0.005% to 0.03% (w/v). In one embodiment, the concentration of the surfactant in the pharmaceutical composition is from 0.005% to 0.025% (w/v). In one embodiment, the concentration of the surfactant in the pharmaceutical composition is from 0.005% to 0.02% (w/v). In one embodiment, the concentration of the surfactant in the pharmaceutical composition is from 0.01% to 0.03% (w/v). In one embodiment, the concentration of the surfactant in the pharmaceutical composition is from 0.01% to 0.025% (w/v). In one embodiment, the concentration of the surfactant in the pharmaceutical composition is from 0.01% to 0.02% (w/v). In one embodiment, the concentration of the surfactant in the pharmaceutical composition is from 0.015% to 0.03% (w/v). In one embodiment, the concentration of the surfactant in the pharmaceutical composition is from 0.015% to 0.025% (w/v). In one embodiment, the concentration of the surfactant in the pharmaceutical composition is from 0.015% to 0.02% (w/v). In one embodiment, the concentration of the surfactant in the pharmaceutical composition is from 0.01% to 0.04% (w/v). In one embodiment, the concentration of the surfactant in the pharmaceutical composition is from 0.01% to 0.05% (w/v). In one embodiment, the concentration of the surfactant in the pharmaceutical composition is from 0.005% to 0.05% (w/v). In one embodiment, the concentration of the surfactant in the pharmaceutical composition is about 0.005% (w/v). In one embodiment, the concentration of the surfactant in the pharmaceutical composition is about 0.01% (w/v). In one embodiment, the concentration of the surfactant in the pharmaceutical composition is about 0.015% (w/v). In one embodiment, the concentration of the surfactant in the pharmaceutical composition is about 0.02% (w/v). In one embodiment, the concentration of the surfactant in the pharmaceutical composition is about 0.025% (w/v). In one embodiment, the concentration of the surfactant in the pharmaceutical composition is about 0.03% (w/v). In one embodiment, the concentration of the surfactant in the pharmaceutical composition is about 0.035% (w/v). In one embodiment, the concentration of the surfactant in the pharmaceutical composition is about 0.04% (w/v). In one embodiment, the concentration of the surfactant in the pharmaceutical composition is about 0.045% (w/v). In one embodiment, the concentration of the surfactant in the pharmaceutical composition is about 0.05% (w/v).

In one embodiment, the concentration of the polysorbate in the pharmaceutical composition is from 0.001% to 0.1% (w/v). In one embodiment, the concentration of the polysorbate in the pharmaceutical composition is from 0.001% to 0.05% (w/v). In one embodiment, the concentration of the polysorbate in the pharmaceutical composition is from 0.001% to 0.02% (w/v). In one embodiment, the concentration of the polysorbate in the pharmaceutical composition is from 0.001% to 0.01% (w/v). In one embodiment, the concentration of the polysorbate in the pharmaceutical composition is from 0.001% to 0.005% (w/v). In one embodiment, the concentration of the polysorbate in the pharmaceutical composition is from 0.005% to 0.01% (w/v). In one embodiment, the concentration of the polysorbate in the pharmaceutical composition is from 0.005% to 0.03% (w/v). In one embodiment, the concentration of the polysorbate in the pharmaceutical composition is from 0.005% to 0.025% (w/v). In one embodiment, the concentration of the polysorbate in the pharmaceutical composition is from 0.005% to 0.02% (w/v). In one embodiment, the concentration of the polysorbate in the pharmaceutical composition is from 0.01% to 0.03% (w/v). In one embodiment, the concentration of the polysorbate in the pharmaceutical composition is from 0.01% to 0.025% (w/v). In one embodiment, the concentration of the polysorbate in the pharmaceutical composition is from 0.01% to 0.02% (w/v). In one embodiment, the concentration of the polysorbate in the pharmaceutical composition is from 0.015% to 0.03% (w/v). In one embodiment, the concentration of the polysorbate in the pharmaceutical composition is from 0.015% to 0.025% (w/v). In one embodiment, the concentration of the polysorbate in the pharmaceutical composition is from 0.015% to 0.02% (w/v). In one embodiment, the concentration of the polysorbate in the pharmaceutical composition is from 0.01% to 0.04% (w/v). In one embodiment, the concentration of the polysorbate in the pharmaceutical composition is from 0.01% to 0.05% (w/v). In one embodiment, the concentration of the polysorbate in the pharmaceutical composition is from 0.005% to 0.05% (w/v). In one embodiment, the concentration of the polysorbate in the pharmaceutical composition is about 0.005% (w/v). In one embodiment, the concentration of the polysorbate in the pharmaceutical composition is about 0.01% (w/v). In one embodiment, the concentration of the polysorbate in the pharmaceutical composition is about 0.015% (w/v). In one embodiment, the concentration of the polysorbate in the pharmaceutical composition is about 0.02% (w/v). In one embodiment, the concentration of the polysorbate in the pharmaceutical composition is about 0.025% (w/v). In one embodiment, the concentration of the polysorbate in the pharmaceutical composition is about 0.03% (w/v). In one embodiment, the concentration of the polysorbate in the pharmaceutical composition is about 0.035% (w/v). In one embodiment, the concentration of the polysorbate in the pharmaceutical composition is about 0.04% (w/v). In one embodiment, the concentration of the polysorbate in the pharmaceutical composition is about 0.045% (w/v). In one embodiment, the concentration of the polysorbate in the pharmaceutical composition is about 0.05% (w/v).

In one embodiment, the concentration of the polysorbate 20 in the pharmaceutical composition is from 0.001% to 0.1% (w/v). In one embodiment, the concentration of the polysorbate 20 in the pharmaceutical composition is from 0.001% to 0.05% (w/v). In one embodiment, the concentration of the polysorbate 20 in the pharmaceutical composition is from 0.001% to 0.02% (w/v). In one embodiment, the concentration of the polysorbate 20 in the pharmaceutical composition is from 0.001% to 0.01% (w/v). In one embodiment, the concentration of the polysorbate 20 in the pharmaceutical composition is from 0.001% to 0.005% (w/v). In one embodiment, the concentration of the polysorbate 20 in the pharmaceutical composition is from 0.005% to 0.01% (w/v). In one embodiment, the concentration of the polysorbate 20 in the pharmaceutical composition is from 0.005% to 0.03% (w/v). In one embodiment, the concentration of the polysorbate 20 in the pharmaceutical composition is from 0.005% to 0.025% (w/v). In one embodiment, the concentration of the polysorbate 20 in the pharmaceutical composition is from 0.005% to 0.02% (w/v). In one embodiment, the concentration of the polysorbate 20 in the pharmaceutical composition is from 0.01% to 0.03% (w/v). In one embodiment, the concentration of the polysorbate 20 in the pharmaceutical composition is from 0.01% to 0.025% (w/v). In one embodiment, the concentration of the polysorbate 20 in the pharmaceutical composition is from 0.01% to 0.02% (w/v). In one embodiment, the concentration of the polysorbate 20 in the pharmaceutical composition is from 0.015% to 0.03% (w/v). In one embodiment, the concentration of the polysorbate 20 in the pharmaceutical composition is from 0.015% to 0.025% (w/v). In one embodiment, the concentration of the polysorbate 20 in the pharmaceutical composition is from 0.015% to 0.02% (w/v). In one embodiment, the concentration of the polysorbate 20 in the pharmaceutical composition is from 0.01% to 0.04% (w/v). In one embodiment, the concentration of the polysorbate 20 in the pharmaceutical composition is from 0.01% to 0.05% (w/v). In one embodiment, the concentration of the polysorbate 20 in the pharmaceutical composition is from 0.005% to 0.05% (w/v). In one embodiment, the concentration of the polysorbate 20 in the pharmaceutical composition is about 0.005% (w/v). In one embodiment, the concentration of the polysorbate 20 in the pharmaceutical composition is about 0.01% (w/v). In one embodiment, the concentration of the polysorbate 20 in the pharmaceutical composition is about 0.015% (w/v). In one embodiment, the concentration of the polysorbate 20 in the pharmaceutical composition is about 0.02% (w/v). In one embodiment, the concentration of the polysorbate 20 in the pharmaceutical composition is about 0.025% (w/v). In one embodiment, the concentration of the polysorbate 20 in the pharmaceutical composition is about 0.03% (w/v). In one embodiment, the concentration of the polysorbate 20 in the pharmaceutical composition is about 0.035% (w/v). In one embodiment, the concentration of the polysorbate 20 in the pharmaceutical composition is about 0.04% (w/v). In one embodiment, the concentration of the polysorbate 20 in the pharmaceutical composition is about 0.045% (w/v). In one embodiment, the concentration of the polysorbate 20 in the pharmaceutical composition is about 0.05% (w/v).

In one embodiment, the concentration of the polysorbate 40 in the pharmaceutical composition is from 0.001% to 0.1% (w/v). In one embodiment, the concentration of the polysorbate 40 in the pharmaceutical composition is from 0.001% to 0.05% (w/v). In one embodiment, the concentration of the polysorbate 40 in the pharmaceutical composition is from 0.001% to 0.02% (w/v). In one embodiment, the concentration of the polysorbate 40 in the pharmaceutical composition is from 0.001% to 0.01% (w/v). In one embodiment, the concentration of the polysorbate 40 in the pharmaceutical composition is from 0.001% to 0.005% (w/v). In one embodiment, the concentration of the polysorbate 40 in the pharmaceutical composition is from 0.005% to 0.01% (w/v). In one embodiment, the concentration of the polysorbate 40 in the pharmaceutical composition is from 0.005% to 0.03% (w/v). In one embodiment, the concentration of the polysorbate 40 in the pharmaceutical composition is from 0.005% to 0.025% (w/v). In one embodiment, the concentration of the polysorbate 40 in the pharmaceutical composition is from 0.005% to 0.02% (w/v). In one embodiment, the concentration of the polysorbate 40 in the pharmaceutical composition is from 0.01% to 0.03% (w/v). In one embodiment, the concentration of the polysorbate 40 in the pharmaceutical composition is from 0.01% to 0.025% (w/v). In one embodiment, the concentration of the polysorbate 40 in the pharmaceutical composition is from 0.01% to 0.02% (w/v). In one embodiment, the concentration of the polysorbate 40 in the pharmaceutical composition is from 0.015% to 0.03% (w/v). In one embodiment, the concentration of the polysorbate 40 in the pharmaceutical composition is from 0.015% to 0.025% (w/v). In one embodiment, the concentration of the polysorbate 40 in the pharmaceutical composition is from 0.015% to 0.02% (w/v). In one embodiment, the concentration of the polysorbate 40 in the pharmaceutical composition is from 0.01% to 0.04% (w/v). In one embodiment, the concentration of the polysorbate 40 in the pharmaceutical composition is from 0.01% to 0.05% (w/v). In one embodiment, the concentration of the polysorbate 40 in the pharmaceutical composition is from 0.005% to 0.05% (w/v). In one embodiment, the concentration of the polysorbate 40 in the pharmaceutical composition is about 0.005% (w/v). In one embodiment, the concentration of the polysorbate 40 in the pharmaceutical composition is about 0.01% (w/v). In one embodiment, the concentration of the polysorbate 40 in the pharmaceutical composition is about 0.015% (w/v). In one embodiment, the concentration of the polysorbate 40 in the pharmaceutical composition is about 0.02% (w/v). In one embodiment, the concentration of the polysorbate 40 in the pharmaceutical composition is about 0.025% (w/v). In one embodiment, the concentration of the polysorbate 40 in the pharmaceutical composition is about 0.03% (w/v). In one embodiment, the concentration of the polysorbate 40 in the pharmaceutical composition is about 0.035% (w/v). In one embodiment, the concentration of the polysorbate 40 in the pharmaceutical composition is about 0.04% (w/v). In one embodiment, the concentration of the polysorbate 40 in the pharmaceutical composition is about 0.045% (w/v). In one embodiment, the concentration of the polysorbate 40 in the pharmaceutical composition is about 0.05% (w/v).

In one embodiment, the concentration of the polysorbate 60 in the pharmaceutical composition is from 0.001% to 0.1% (w/v). In one embodiment, the concentration of the polysorbate 60 in the pharmaceutical composition is from 0.001% to 0.05% (w/v). In one embodiment, the concentration of the polysorbate 60 in the pharmaceutical composition is from 0.001% to 0.02% (w/v). In one embodiment, the concentration of the polysorbate 60 in the pharmaceutical composition is from 0.001% to 0.01% (w/v). In one embodiment, the concentration of the polysorbate 60 in the pharmaceutical composition is from 0.001% to 0.005% (w/v). In one embodiment, the concentration of the polysorbate 60 in the pharmaceutical composition is from 0.005% to 0.01% (w/v). In one embodiment, the concentration of the polysorbate 60 in the pharmaceutical composition is from 0.005% to 0.03% (w/v). In one embodiment, the concentration of the polysorbate 60 in the pharmaceutical composition is from 0.005% to 0.025% (w/v). In one embodiment, the concentration of the polysorbate 60 in the pharmaceutical composition is from 0.005% to 0.02% (w/v). In one embodiment, the concentration of the polysorbate 60 in the pharmaceutical composition is from 0.01% to 0.03% (w/v). In one embodiment, the concentration of the polysorbate 60 in the pharmaceutical composition is from 0.01% to 0.025% (w/v). In one embodiment, the concentration of the polysorbate 60 in the pharmaceutical composition is from 0.01% to 0.02% (w/v). In one embodiment, the concentration of the polysorbate 60 in the pharmaceutical composition is from 0.015% to 0.03% (w/v). In one embodiment, the concentration of the polysorbate 60 in the pharmaceutical composition is from 0.015% to 0.025% (w/v). In one embodiment, the concentration of the polysorbate 60 in the pharmaceutical composition is from 0.015% to 0.02% (w/v). In one embodiment, the concentration of the polysorbate 60 in the pharmaceutical composition is from 0.01% to 0.04% (w/v). In one embodiment, the concentration of the polysorbate 60 in the pharmaceutical composition is from 0.01% to 0.05% (w/v). In one embodiment, the concentration of the polysorbate 60 in the pharmaceutical composition is from 0.005% to 0.05% (w/v). In one embodiment, the concentration of the polysorbate 60 in the pharmaceutical composition is about 0.005% (w/v). In one embodiment, the concentration of the polysorbate 60 in the pharmaceutical composition is about 0.01% (w/v). In one embodiment, the concentration of the polysorbate 60 in the pharmaceutical composition is about 0.015% (w/v). In one embodiment, the concentration of the polysorbate 60 in the pharmaceutical composition is about 0.02% (w/v). In one embodiment, the concentration of the polysorbate 60 in the pharmaceutical composition is about 0.025% (w/v). In one embodiment, the concentration of the polysorbate 60 in the pharmaceutical composition is about 0.03% (w/v). In one embodiment, the concentration of the polysorbate 60 in the pharmaceutical composition is about 0.035% (w/v). In one embodiment, the concentration of the polysorbate 60 in the pharmaceutical composition is about 0.04% (w/v). In one embodiment, the concentration of the polysorbate 60 in the pharmaceutical composition is about 0.045% (w/v). In one embodiment, the concentration of the polysorbate 60 in the pharmaceutical composition is about 0.05% (w/v).

In one embodiment, the concentration of the polysorbate 80 in the pharmaceutical composition is from 0.001% to 0.1% (w/v). In one embodiment, the concentration of the polysorbate 80 in the pharmaceutical composition is from 0.001% to 0.05% (w/v). In one embodiment, the concentration of the polysorbate 80 in the pharmaceutical composition is from 0.001% to 0.02% (w/v). In one embodiment, the concentration of the polysorbate 80 in the pharmaceutical composition is from 0.001% to 0.01% (w/v). In one embodiment, the concentration of the polysorbate 80 in the pharmaceutical composition is from 0.001% to 0.005% (w/v). In one embodiment, the concentration of the polysorbate 80 in the pharmaceutical composition is from 0.005% to 0.01% (w/v). In one embodiment, the concentration of the polysorbate 80 in the pharmaceutical composition is from 0.005% to 0.03% (w/v). In one embodiment, the concentration of the polysorbate 80 in the pharmaceutical composition is from 0.005% to 0.025% (w/v). In one embodiment, the concentration of the polysorbate 80 in the pharmaceutical composition is from 0.005% to 0.02% (w/v). In one embodiment, the concentration of the polysorbate 80 in the pharmaceutical composition is from 0.01% to 0.03% (w/v). In one embodiment, the concentration of the polysorbate 80 in the pharmaceutical composition is from 0.01% to 0.025% (w/v). In one embodiment, the concentration of the polysorbate 80 in the pharmaceutical composition is from 0.01% to 0.02% (w/v). In one embodiment, the concentration of the polysorbate 80 in the pharmaceutical composition is from 0.015% to 0.03% (w/v). In one embodiment, the concentration of the polysorbate 80 in the pharmaceutical composition is from 0.015% to 0.025% (w/v). In one embodiment, the concentration of the polysorbate 80 in the pharmaceutical composition is from 0.015% to 0.02% (w/v). In one embodiment, the concentration of the polysorbate 80 in the pharmaceutical composition is from 0.01% to 0.04% (w/v). In one embodiment, the concentration of the polysorbate 80 in the pharmaceutical composition is from 0.01% to 0.05% (w/v). In one embodiment, the concentration of the polysorbate 80 in the pharmaceutical composition is from 0.005% to 0.05% (w/v). In one embodiment, the concentration of the polysorbate 80 in the pharmaceutical composition is about 0.005% (w/v). In one embodiment, the concentration of the polysorbate 80 in the pharmaceutical composition is about 0.01% (w/v). In one embodiment, the concentration of the polysorbate 80 in the pharmaceutical composition is about 0.015% (w/v). In one embodiment, the concentration of the polysorbate 80 in the pharmaceutical composition is about 0.02% (w/v). In one embodiment, the concentration of the polysorbate 80 in the pharmaceutical composition is about 0.025% (w/v). In one embodiment, the concentration of the polysorbate 80 in the pharmaceutical composition is about 0.03% (w/v). In one embodiment, the concentration of the polysorbate 80 in the pharmaceutical composition is about 0.035% (w/v). In one embodiment, the concentration of the polysorbate 80 in the pharmaceutical composition is about 0.04% (w/v). In one embodiment, the concentration of the polysorbate 80 in the pharmaceutical composition is about 0.045% (w/v). In one embodiment, the concentration of the polysorbate 80 in the pharmaceutical composition is about 0.05% (w/v).

In one embodiment, the concentration of the poloxamer 188 in the pharmaceutical composition is from 0.001% to 0.1% (w/v). In one embodiment, the concentration of the poloxamer 188 in the pharmaceutical composition is from 0.001% to 0.05% (w/v). In one embodiment, the concentration of the poloxamer 188 in the pharmaceutical composition is from 0.001% to 0.02% (w/v). In one embodiment, the concentration of the poloxamer 188 in the pharmaceutical composition is from 0.001% to 0.01% (w/v). In one embodiment, the concentration of the poloxamer 188 in the pharmaceutical composition is from 0.001% to 0.005% (w/v). In one embodiment, the concentration of the poloxamer 188 in the pharmaceutical composition is from 0.005% to 0.01% (w/v). In one embodiment, the concentration of the poloxamer 188 in the pharmaceutical composition is from 0.005% to 0.03% (w/v). In one embodiment, the concentration of the poloxamer 188 in the pharmaceutical composition is from 0.005% to 0.025% (w/v). In one embodiment, the concentration of the poloxamer 188 in the pharmaceutical composition is from 0.005% to 0.02% (w/v). In one embodiment, the concentration of the poloxamer 188 in the pharmaceutical composition is from 0.01% to 0.03% (w/v). In one embodiment, the concentration of the poloxamer 188 in the pharmaceutical composition is from 0.01% to 0.025% (w/v). In one embodiment, the concentration of the poloxamer 188 in the pharmaceutical composition is from 0.01% to 0.02% (w/v). In one embodiment, the concentration of the poloxamer 188 in the pharmaceutical composition is from 0.015% to 0.03% (w/v). In one embodiment, the concentration of the poloxamer 188 in the pharmaceutical composition is from 0.015% to 0.025% (w/v). In one embodiment, the concentration of the poloxamer 188 in the pharmaceutical composition is from 0.015% to 0.02% (w/v). In one embodiment, the concentration of the poloxamer 188 in the pharmaceutical composition is from 0.01% to 0.04% (w/v). In one embodiment, the concentration of the poloxamer 188 in the pharmaceutical composition is from 0.01% to 0.05% (w/v). In one embodiment, the concentration of the poloxamer 188 in the pharmaceutical composition is from 0.005% to 0.05% (w/v). In one embodiment, the concentration of the poloxamer 188 in the pharmaceutical composition is about 0.005% (w/v). In one embodiment, the concentration of the poloxamer 188 in the pharmaceutical composition is about 0.01% (w/v). In one embodiment, the concentration of the poloxamer 188 in the pharmaceutical composition is about 0.015% (w/v). In one embodiment, the concentration of the poloxamer 188 in the pharmaceutical composition is about 0.02% (w/v). In one embodiment, the concentration of the poloxamer 188 in the pharmaceutical composition is about 0.025% (w/v). In one embodiment, the concentration of the poloxamer 188 in the pharmaceutical composition is about 0.03% (w/v). In one embodiment, the concentration of the poloxamer 188 in the pharmaceutical composition is about 0.035% (w/v). In one embodiment, the concentration of the poloxamer 188 in the pharmaceutical composition is about 0.04% (w/v). In one embodiment, the concentration of the poloxamer 188 in the pharmaceutical composition is about 0.045% (w/v). In one embodiment, the concentration of the poloxamer 188 in the pharmaceutical composition is about 0.05% (w/v).

### 4.5.5 Salt in the Pharmaceutical Composition Comprising Anti-CD30L Antibody or Antigen Binding Fragment

Certain embodiments of the salt in the pharmaceutical compositions comprising anti-CD30L provided in Section 4.5 are described and provided in this Section 4.5.5. Accordingly, in certain embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition comprises a buffer provided in Section 4.5.2, wherein the pharmaceutical composition comprises a pharmaceutically acceptable excipient provided in Section 4.5.3, wherein the pharmaceutical composition comprises a surfactant provided in Section 4.5.4, and wherein the pharmaceutical composition comprises a salt provided in this Section 4.5.5. In some embodiments, provided herein is a pharmaceutical composition comprising an anti-CD30L antibody or antigen binding fragment thereof provided in Sections 2 and 4.1, wherein the pharmaceutical composition comprises a salt provided in this Section 4.5.5. In some embodiments, provided herein is a pharmaceutical composition comprising an anti-CD30L antibody or antigen binding fragment thereof provided in Sections 2 and 4.1, wherein the pharmaceutical composition comprises a surfactant provided in Section 4.5.4 and wherein the pharmaceutical composition comprises a salt provided in this Section 4.5.5. In certain embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition comprises a salt provided in this Section 4.5.5. In certain embodiments, provided herein is a pharmaceutical composition comprising an anti-CD30L antibody or antigen binding fragment thereof provided in Sections 2 and 4.1, wherein the pharmaceutical composition comprises a buffer provided in Section 4.5.2, and wherein the pharmaceutical composition comprises a salt provided in this Section 4.5.5. In some embodiments, provided herein is a pharmaceutical composition comprising an anti-CD30L antibody or antigen binding fragment thereof provided in Sections 2 and 4.1, wherein the pharmaceutical composition comprises a pharmaceutically acceptable excipient provided in Section 4.5.3, and wherein the pharmaceutical composition comprises a salt provided in this Section 4.5.5. In certain embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition comprises a surfactant provided in Section 4.5.4, and wherein the pharmaceutical composition comprises a salt provided in this Section 4.5.5. In certain embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition comprises a buffer provided in Section 4.5.2, and wherein the pharmaceutical composition comprises a salt provided in this Section 4.5.5. In certain embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition comprises a pharmaceutically acceptable excipient provided in Section 4.5.3, and wherein the pharmaceutical composition comprises a salt provided in this Section 4.5.5. In certain embodiments, provided herein is a pharmaceutical composition comprising an anti-CD30L antibody or antigen binding fragment thereof provided in Sections 2 and 4.1, wherein the pharmaceutical composition comprises a buffer provided in Section 4.5.2, wherein the pharmaceutical composition comprises a pharmaceutically acceptable excipient provided in Section 4.5.3, and wherein the pharmaceutical composition comprises a salt provided in this Section 4.5.5. In certain embodiments, provided herein is a pharmaceutical composition comprising an anti-CD30L antibody or antigen binding fragment thereof provided in Sections 2 and 4.1, wherein the pharmaceutical composition comprises a buffer provided in Section 4.5.2, wherein the pharmaceutical composition comprises a surfactant provided in Section 4.5.4, and wherein the pharmaceutical composition comprises a salt provided in this Section 4.5.5. In some embodiments, provided herein is a pharmaceutical composition comprising an anti-CD30L antibody or antigen binding fragment thereof provided in Sections 2 and 4.1, wherein the pharmaceutical composition comprises a pharmaceutically acceptable excipient provided in Section 4.5.3, wherein the pharmaceutical composition comprises a surfactant provided in Section 4.5.4, and wherein the pharmaceutical composition comprises a salt provided in this Section 4.5.5. In some embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition comprises a buffer provided in Section 4.5.2, wherein the pharmaceutical composition comprises a pharmaceutically acceptable excipient provided in Section 4.5.3, and wherein the pharmaceutical composition comprises a salt provided in this Section 4.5.5. In some embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition comprises a buffer provided in Section 4.5.2, wherein the pharmaceutical composition comprises a surfactant provided in Section 4.5.4, and wherein the pharmaceutical composition comprises a salt provided in this Section 4.5.5. In some embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition comprises a pharmaceutically acceptable excipient provided in Section 4.5.3, wherein the pharmaceutical composition comprises a surfactant provided in Section 4.5.4, and wherein the pharmaceutical composition comprises a salt provided in this Section 4.5.5. In some embodiments, provided herein is a pharmaceutical composition comprising an anti-CD30L antibody or antigen binding fragment thereof provided in Sections 2 and 4.1, wherein the pharmaceutical composition comprises a buffer provided in Section 4.5.2, wherein the pharmaceutical composition comprises a pharmaceutically acceptable excipient provided in Section 4.5.3, wherein the pharmaceutical composition comprises a surfactant provided in Section 4.5.4, and wherein the pharmaceutical composition comprises a salt provided in this Section 4.5.5. As is clear from the description herein, the disclosure provides that the excipient provided in Section 4.5.3 for the pharmaceutical composition can be alternative to or additional to the salt provided in this Section 4.5.5 for the pharmaceutical composition.

In some embodiments, the salt is present in the composition at a concentration of about 10 mM to about 150 mM. For instance, the salt is present at a concentration of about 10 mM to about 150 mM, about 10 mM to about 140 mM, about 10 mM to about 130 mM, about 10 mM to about 120 mM, about 10 mM to about 110 mM, about 10 mM to about 100 mM, about 10 mM to about 90 mM, about 10 mM to about 80 mM, about 10 mM to about 70 mM, about 10 mM to about 60 mM, about 10 mM to about 50 mM, about 10 mM to about 40 mM, about 10 mM to about 30 mM, about 20 mM to about 150 mM, about 20 mM to about 140 mM, about 20 mM to about 130 mM, about 20 mM to about 120 mM, about 20 mM to about 110 mM, about 20 mM to about 100 mM, about 20 mM to about 90 mM, about 20 mM to about 80 mM, about 20 mM to about 70 mM, about 20 mM to about 60 mM, about 20 mM to about 50 mM, about 20 mM to about 40 mM, about 20 mM to about 30 mM, about 30 mM to about 150 mM, about 30 mM to about 140 mM, about 30 mM to about 130 mM, about 30 mM to about 120 mM, about 30 mM to about 110 mM, about 30 mM to about 100 mM, about 30 mM to about 90 mM, about 30 mM to about 80 mM, about 30 mM to about 70 mM, about 30 mM to about 60 mM, about 30 mM to about 50 mM, about 30 mM to about 40 mM, about 40 mM to about 150 mM, about 40 mM to about 140 mM, about 40 mM to about 130 mM, about 40 mM to about 120 mM, about 40 mM to about 110 mM, about 40 mM to about 100 mM, about 40 mM to about 90 mM, about 40 mM to about 80 mM, about 40 mM to about 70 mM, about 40 mM to about 60 mM, or about 40 mM to about 50 mM. In example embodiments, the salt is present at concentrations of about 25 mM to about 130 mM. In some embodiments, the salt in the pharmaceutical composition is NaCl (sodium chloride). As a further embodiment, the composition comprises about 40 mM to about 130 mM NaCl. For instance, the composition comprises about 40 mM NaCl. In some embodiments, the composition comprises about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, or about 150 mM NaCl.

### 4.5.6 Volume of the Pharmaceutical Composition Comprising Anti-CD30L Antibody or Antigen Binding Fragment

Certain embodiments of the volume of the pharmaceutical compositions comprising anti-CD30L provided in Section 4.5 are described and provided in this Section 4.5.6. Accordingly, in certain embodiments, provided herein is a pharmaceutical composition comprising an anti-CD30L antibody or antigen binding fragment thereof provided in Sections 2 and 4.1, for example as provided in Sections 4.5.1, 4.5.2, 4.5.3, 4.5.4, and/or 4.5.5, wherein the volume of the pharmaceutical composition can be any one of those described in this Section 4.5.6.

In certain embodiments, provided herein is a pharmaceutical composition comprising a therapeutically effective dose of an anti-CD30L antibody or antigen binding fragment having a total volume of less than or equal to about 2.5 mL. In some embodiments, the pharmaceutical composition comprises a therapeutically effective dose of an anti-CD30L antibody or antigen binding fragment having a total volume of less than or equal to about 2 mL. The total volume may be less than or equal to about 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, or 0.8 mL. The total volume may be at least about 0.5 mL. The total volume may be about 0.5 mL to about 3 mL, about 0.5 mL to about 2.9 mL, about 0.5 mL to about 2.8 mL, about 0.5 mL to about 2.7 mL, about 0.5 mL to about 2.6 mL, about 0.5 mL to about 2.5 mL, about 0.5 mL to about 2.4 mL, about 0.5 mL to about 2.3 mL, about 0.5 mL to about 2.2 mL, about 0.5 mL to about 2.1 mL, about 0.5 mL to about 2.0 mL, about 0.5 mL to about 1.9 mL, about 0.5 mL to about 1.8 mL, about 0.5 mL to about 1.7 mL, about 0.5 mL to about 1.6 mL, about 0.5 mL to about 1.5 mL, about 0.5 mL to about 1.4 mL, about 0.5 mL to about 1.3 mL, about 0.5 mL to about 1.2 mL, about 0.5 mL to about 1.1 mL, about 0.5 mL to about 1.0 mL, about 0.5 mL to about 0.9 mL, about 0.5 mL to about 0.8 mL, about 0.6 mL to about 3 mL, about 0.6 mL to about 2.9 mL, about 0.6 mL to about 2.8 mL, about 0.6 mL to about 2.7 mL, about 0.6 mL to about 2.6 mL, about 0.6 mL to about 2.5 mL, about 0.6 mL to about 2.4 mL, about 0.6 mL to about 2.3 mL, about 0.6 mL to about 2.2 mL, about 0.6 mL to about 2.1 mL, about 0.6 mL to about 2.0 mL, about 0.6 mL to about 1.9 mL, about 0.6 mL to about 1.8 mL, about 0.6 mL to about 1.7 mL, about 0.6 mL to about 1.6 mL, 0.6 mL to about 1.5 mL, about 0.6 mL to about 1.4 mL, about 0.6 mL to about 1.3 mL, about 0.6 mL to about 1.2 mL, about 0.6 mL to about 1.1 mL, about 0.6 mL to about 1.0 mL, about 0.6 mL to about 0.9 mL, about 0.6 mL to about 0.8 mL, about 0.7 mL to about 3 mL, about 0.7 mL to about 2.9 mL, about 0.7 mL to about 2.8 mL, about 0.7 mL to about 2.7 mL, about 0.7 mL to about 2.6 mL, about 0.7 mL to about 2.5 mL, about 0.7 mL to about 2.4 mL, about 0.7 mL to about 2.3 mL, about 0.7 mL to about 2.2 mL, about 0.7 mL to about 2.1 mL, about 0.7 mL to about 2.0 mL, about 0.7 mL to about 1.9 mL, about 0.7 mL to about 1.8 mL, about 0.7 mL to about 1.7 mL, about 0.7 mL to about 1.6 mL, about 0.7 mL to about 1.5 mL, about 0.7 mL to about 1.4 mL, about 0.7 mL to about 1.3 mL, about 0.7 mL to about 1.2 mL, about 0.7 mL to about 1.1 mL, about 0.7 mL to about 1.0 mL, about 0.7 mL to about 0.9 mL, about 0.7 mL to about 0.8 mL, about 3 mL to about 10 mL, about 3 mL to about 9.5 mL, about 3 mL to about 9.0 mL, about 3 mL to about 8.5 mL, about 3 mL to about 8.0 mL, about 3 mL to about 7.5 mL, about 3 mL to about 7.0 mL, about 3 mL to about 6.5 mL, about 3 mL to about 6 mL, about 3 mL to about 5.5 mL, about 3 mL to about 5.0 mL, about 3 mL to about 4.5 mL, about 3 mL to about 4 mL, about 3 mL to about 3.5 mL, about 3.5 mL to about 10 mL, about 3.5 mL to about 9.5 mL, about 3.5 mL to about 9.0 mL, about 3.5 mL to about 8.5 mL, about 3.5 mL to about 8.0 mL, about 3.5 mL to about 7.5 mL, about 3.5 mL to about 7.0 mL, about 3.5 mL to about 6.5 mL, about 3.5 mL to about 6 mL, about 3.5 mL to about 5.5 mL, about 3.5 mL to about 5.0 mL, about 3.5 mL to about 4.5 mL, about 3.5 mL to about 4 mL, about 4.0 mL to about 10 mL, about 4.0 mL to about 9.5 mL, about 4.0 mL to about 9.0 mL, about 4.0 mL to about 8.5 mL, about 4.0 mL to about 8.0 mL, about 4.0 mL to about 7.5 mL, about 4.0 mL to about 7.0 mL, about 4.0 mL to about 6.5 mL, about 4.0 mL to about 6 mL, about 4.0 mL to about 5.5 mL, about 4.0 mL to about 5.0 mL, about 4.0 mL to about 4.5 mL, about 4.5 mL to about 10 mL, about 4.5 mL to about 9.5 mL, about 4.5 mL to about 9.0 mL, about 4.5 mL to about 8.5 mL, about 4.5 mL to about 8.0 mL, about 4.5 mL to about 7.5 mL, about 4.5 mL to about 7.0 mL, about 4.5 mL to about 6.5 mL, about 4.5 mL to about 6 mL, about 4.5 mL to about 5.5 mL, about 4.5 mL to about 5.0 mL, about 5 mL to about 10 mL, about 5 mL to about 9.5 mL, about 5 mL to about 9.0 mL, about 5 mL to about 8.5 mL, about 5 mL to about 8.0 mL, about 5 mL to about 7.5 mL, about 5 mL to about 7.0 mL, about 5 mL to about 6.5 mL, about 5 mL to about 6 mL, about 5 mL to about 5.5 mL, about 5.5 mL to about 10 mL, about 5.5 mL to about 9.5 mL, about 5.5 mL to about 9.0 mL, about 5.5 mL to about 8.5 mL, about 5.5 mL to about 8.0 mL, about 5.5 mL to about 7.5 mL, about 5.5 mL to about 7.0 mL, about 5.5 mL to about 6.5 mL, about 5.5 mL to about 6 mL, about 6.0 mL to about 10 mL, about 6.0 mL to about 9.5 mL, about 6.0 mL to about 9.0 mL, about 6.0 mL to about 8.5 mL, about 6.0 mL to about 8.0 mL, about 6.0 mL to about 7.5 mL, about 6.0 mL to about 7.0 mL, about 6.0 mL to about 6.5 mL, about 6.5 mL to about 10 mL, about 6.5 mL to about 9.5 mL, about 6.5 mL to about 9.0 mL, about 6.5 mL to about 8.5 mL, about 6.5 mL to about 8.0 mL, about 6.5 mL to about 7.5 mL, about 6.5 mL to about 7.0 mL, about 7.0 mL to about 10 mL, about 7.0 mL to about 9.5 mL, about 7.0 mL to about 9.0 mL, about 7.0 mL to about 8.5 mL, about 7.0 mL to about 8.0 mL, about 7.0 mL to about 7.5 mL, about 7.5 mL to about 10 mL, about 7.5 mL to about 9.5 mL, about 7.5 mL to about 9.0 mL, about 7.5 mL to about 8.5 mL, about 7.5 mL to about 8.0 mL, about 8.0 mL to about 10 mL, about 8.0 mL to about 9.5 mL, about 8.0 mL to about 9.0 mL, about 8.0 mL to about 8.5 mL, about 8.5 mL to about 10 mL, about 8.5 mL to about 9.5 mL, about 8.5 mL to about 9.0 mL, about 9 mL to about 10 mL, about 9 mL to about 9.5 mL, or about 9.5 mL to about 10 mL.

### 4.5.7 Stability and Other Properties of the Pharmaceutical Composition Comprising Anti-CD30L Antibody or Antigen Binding Fragment

The pharmaceutical compositions provided herein are formulated to achieve pharmaceutically desirable characteristics, including but not limited to stability, viscosity, and safety. As such the disclosure provides that the pharmaceutical composition of the anti-CD30L provided in Section 4.5 can have the stability characteristics and/or other characteristics as provided in this Section 4.5.7. Accordingly, in certain embodiments, provided herein is a pharmaceutical composition comprising an anti-CD30L antibody or antigen binding fragment thereof provided in Sections 2 and 4.1, for example as provided in Sections 4.5.1, 4.5.2, 4.5.3, 4.5.4, 4.5.5, and/or 4.5.6, wherein the pharmaceutical composition has the stability characteristics and/or other characteristics as provided in this Section 4.5.7.

In certain embodiments, the various pharmaceutical compositions provided herein are stable. In some embodiments, the various pharmaceutical compositions provided herein are in liquid formulation and are stable. Stability of the pharmaceutical compositions provided herein can be measured at a selected temperature for a selected time period. In one embodiment, the antibody in the liquid formulations is stable in a liquid form for at least about 2 weeks. In one embodiment, the antibody in the liquid formulations is stable in a liquid form for at least about 4 weeks. In one embodiment, the antibody in the liquid formulations is stable in a liquid form for at least about 8 weeks. In one embodiment, the antibody in the liquid formulations is stable in a liquid form for at least about 12 weeks. In one embodiment, the antibody in the liquid formulations is stable in a liquid form for at least about 26 weeks. In one embodiment, the antibody in the liquid formulations is stable in a liquid form for at least about 14 months. In one embodiment, the antibody in the liquid formulations is stable in a liquid form for at least about 3 months. In one embodiment, the antibody in the liquid formulations is stable in a liquid form for at least about 4 months. In one embodiment, the antibody in the liquid formulations is stable in a liquid form for at least about 5 months. In one embodiment, the antibody in the liquid formulations is stable in a liquid form for at least about 6 months. In one embodiment, the antibody in the liquid formulations is stable in a liquid form for at least about 12 months. In one embodiment, the antibody in the liquid formulations is stable in a liquid form for at least about 18 months. In one embodiment, the antibody in the liquid formulations is stable in a liquid form for at least about 24 months. Values and ranges intermediate to the above recited time periods are also contemplated, e.g., about 3, 4, 5, 6, 7, 8, 9, 10, 11, 12, 13, 14, 15, 16, 17, 18, 19, 20, 21, 22, 23, or 24 months. In addition, ranges of values using a combination of any of the above recited values as upper and/or lower limits are intended to be included. In some embodiments, the pharmaceutical composition is stable at −70° C. In some embodiments, the pharmaceutical composition is stable at 4° C. In some embodiments, the pharmaceutical composition is stable at 5° C. In some embodiments, the pharmaceutical composition is stable at 25° C. In some embodiments, the pharmaceutical composition is stable at 30° C. In some embodiments, the pharmaceutical composition is stable at 40° C. In some embodiments, the pharmaceutical composition is stable in the presence of 0.1-5.0 ppm H2O2 at 25±5° C.

In one embodiment, the formulated antibodies provided herein remain stable over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months or longer at 5±3° C. In another embodiment, the formulated antibodies provided herein remain stable over 1 week, 2 weeks, 4 weeks, 8 weeks, 2 months, 3 months, 4 months, 5 months, 6 months, 7 months, 8 months, 9 months, 10 months, 11 months, 12 months, 18 months, 24 months, or longer at 25±5° C. In yet another embodiment, the formulated antibodies provided herein remain stable over 1 week, 2 weeks, 4 weeks, 8 weeks, 2 months, 3 months, 4 months, 5 months, 6 months, 7 months, 8 months, 9 months, 10 months, 11 months, 12 months, 18 months, 24 months, 30 months, 36 months, 48 months, or longer at 40±5° C. In yet another embodiment, the formulated antibodies provided herein remain stable over 1 week, 2 weeks, 4 weeks, 8 weeks, 2 months, 3 months, 4 months, 5 months, 6 months, 7 months, 8 months, 9 months, 10 months, 11 months, 12 months, 18 months, 24 months, 30 months, 36 months, 48 months, or longer at −70±10° C. In yet another embodiment, the formulated antibodies provided herein remain stable after 1, 2, 3, 4, 5, 6, 7, 8, 9, 10 or more daily freeze/thaw cycles at −80° C./5° C. In still another embodiment, the formulated antibodies provided herein remain stable after vortexing continuously for 1, 2, 3, 4, 5, 6, 7, 8, 9, 10, 15, 20, 30 or more days at 5° C. In still another embodiment, the formulated antibodies provided herein remain stable after vortexing continuously for 1, 2, 3, 4, 5, 6, 7, 8, 9, 10, 11, 12, 13, 14, 15, 16, 17, 18, 19, 20, 21, 22, 23, 24 or more hours at 5° C.±3° C. In some embodiments, the formulated antibodies provided herein remain stable after agitating continuously for 1, 2, 3, 4, 5, 6, 7, 8, 9, 10, 15, 20, 30 or more days at 5° C. In still another embodiment, the formulated antibodies provided herein remain stable after agitating continuously for 1, 2, 3, 4, 5, 6, 7, 8, 9, 10, 11, 12, 13, 14, 15, 16, 17, 18, 19, 20, 21, 22, 23, 24 or more hours at 5° C.±3° C. In some embodiments, the formulated antibodies provided herein remain stable after agitating continuously for 1, 2, 3, 4, 5, 6, 7, 8, 9, 10, 15, 20, 30 or more days at 25° C. or room temperature. In some embodiments, the formulated antibodies provided herein remain stable after agitating continuously for 1, 2, 3, 4, 5, 6, 7, 8, 9, 10, 11, 12, 13, 14, 15, 16, 17, 18, 19, 20, 21, 22, 23, 24 or more hours at 25° C.±3° C. In some embodiments, the formulated antibodies provided herein remain stable after agitating continuously for 1, 2, 3, 4, 5, 6, 7, 8, 9, 10, 15, 20, 30 or more days at 37° C. In some embodiments, the formulated antibodies provided herein remain stable after agitating continuously for 1, 2, 3, 4, 5, 6, 7, 8, 9, 10, 11, 12, 13, 14, 15, 16, 17, 18, 19, 20, 21, 22, 23, 24 or more hours at 37° C.±3° C. In some embodiments, the formulated antibodies provided herein remain stable after agitating at 100 RPM continuously for 1, 2, 3, 4, 5, 6, 7, 8, 9, 10, 15, 20, 30 or more days at 25° C. or room temperature. In some embodiments, the formulated antibodies provided herein remain stable after agitating at 100 RPM continuously for 1, 2, 3, 4, 5, 6, 7, 8, 9, 10, 11, 12, 13, 14, 15, 16, 17, 18, 19, 20, 21, 22, 23, 24 or more hours at 25° C.±3° C. In some embodiments, the formulated antibodies provided herein remain stable after agitating at 100 RPM continuously for 1, 2, 3, 4, 5, 6, 7, 8, 9, 10, 15, 20, 30 or more days at 37° C. In some embodiments, the formulated antibodies provided herein remain stable after agitating at 100 RPM continuously for 1, 2, 3, 4, 5, 6, 7, 8, 9, 10, 11, 12, 13, 14, 15, 16, 17, 18, 19, 20, 21, 22, 23, 24 or more hours at 37° C.±3° C. In some embodiments, the formulated antibodies provided herein remain stable after agitating at 300 RPM continuously for 1, 2, 3, 4, 5, 6, 7, 8, 9, 10, 15, 20, 30 or more days at 25° C. or room temperature. In some embodiments, the formulated antibodies provided herein remain stable after agitating at 300 RPM continuously for 1, 2, 3, 4, 5, 6, 7, 8, 9, 10, 11, 12, 13, 14, 15, 16, 17, 18, 19, 20, 21, 22, 23, 24 or more hours at 25° C.±3° C. In some embodiments, the formulated antibodies provided herein remain stable after agitating at 300 RPM continuously for 1, 2, 3, 4, 5, 6, 7, 8, 9, 10, 15, 20, 30 or more days at 37° C. In some embodiments, the formulated antibodies provided herein remain stable after vigorously agitating at 300 RPM continuously for 1, 2, 3, 4, 5, 6, 7, 8, 9, 10, 11, 12, 13, 14, 15, 16, 17, 18, 19, 20, 21, 22, 23, 24 or more hours at 37° C.±3° C. In some embodiment, the formulated antibodies provided herein remain stable after agitating continuously for 3, 5 or 10 days at 25° C. or room temperature. In some embodiment, the formulated antibodies provided herein remain stable after agitating at 100 RPM continuously for 3, 5 or 10 days at 25° C. or room temperature. In some embodiment, the formulated antibodies provided herein remain stable after agitating at 300 RPM continuously for 3, 5 or 10 days at 25° C. or room temperature. In some embodiment, the formulated antibodies provided herein remain stable after agitating continuously for 3, 5 or 10 days at 37° C. In some embodiment, the formulated antibodies provided herein remain stable after agitating at 100 RPM continuously for 3, 5 or 10 days at 37° C. In some embodiment, the formulated antibodies provided herein remain stable after agitating at 300 RPM continuously for 3, 5 or 10 days at 37° C. In some embodiment, the formulated antibodies provided herein remain stable after 1 cycle of freeze and thaw from −70° C. to room temperature (or 25° C.). In some embodiment, the formulated antibodies provided herein remain stable after 2 cycles of freeze and thaw from −70° C. to room temperature (or 25° C.). In some embodiment, the formulated antibodies provided herein remain stable after 3 cycles of freeze and thaw from −70° C. to room temperature (or 25° C.). In some embodiment, the formulated antibodies provided herein remain stable after 4 cycles of freeze and thaw from −70° C. to room temperature (or 25° C.). In some embodiment, the formulated antibodies provided herein remain stable after 5 cycles of freeze and thaw from −70° C. to room temperature (or 25° C.). In some embodiment, the formulated antibodies provided herein remain stable after 6 cycles of freeze and thaw from −70° C. to room temperature (or 25° C.). In some embodiment, the formulated antibodies provided herein remain stable after 7 cycles of freeze and thaw from −70° C. to room temperature (or 25° C.). In some embodiment, the formulated antibodies provided herein remain stable after 8 cycles of freeze and thaw from −70° C. to room temperature (or 25° C.). In some embodiment, the formulated antibodies provided herein remain stable after 9 cycles of freeze and thaw from −70° C. to room temperature (or 25° C.). In some embodiment, the formulated antibodies provided herein remain stable after 10 cycles of freeze and thaw from −70° C. to room temperature (or 25° C.). In a specific embodiment, the pharmaceutical composition is stable for at least 12 months when stored at −70° C.±10° C. In other embodiments, the pharmaceutical composition is stable for at least 6 months when stored at 5° C.±3° C. In one embodiment, the formulated antibodies provided herein remain stable for at least 2 weeks when stored at 5±3° C. In one embodiment, the formulated antibodies provided herein remain stable for at least 4 weeks when stored at 5±3° C. In one embodiment, the formulated antibodies provided herein remain stable for at least 12 weeks when stored at 5±3° C. In other embodiments, the pharmaceutical composition is stable for at least 6 months when stored in the presence of 0.1-5.0 ppm H2O2 at 25±5° C. In one embodiment, the formulated antibodies provided herein remain stable for at least 2 weeks when stored in the presence of 0.1-5.0 ppm H2O2 at 25±5° C. In one embodiment, the formulated antibodies provided herein remain stable for at least 4 weeks when stored in the presence of 0.1-5.0 ppm H2O2 at 25±5° C. In one embodiment, the formulated antibodies provided herein remain stable for at least 12 weeks when stored in the presence of 0.1-5.0 ppm H2O2 at 25±5° C. In one embodiment, the formulated antibodies provided herein remain stable for at least 2 weeks when stored at 25±5° C. In one embodiment, the formulated antibodies provided herein remain stable for at least 4 weeks when stored at 25±5° C. In one embodiment, the formulated antibodies provided herein remain stable for at least 12 weeks when stored at 25±5° C. In one embodiment, the formulated antibodies provided herein remain stable for at least 2 weeks when stored at 40±5° C. In one embodiment, the formulated antibodies provided herein remain stable for at least 4 weeks when stored at 40±5° C. In one embodiment, the formulated antibodies provided herein remain stable for at least 12 weeks when stored at 40±5° C.

The stability of the anti-CD30L compositions provided herein can be determined by the content or percentage of the anti-CD30L monomer. For clarity, anti-CD30L monomer or monomer when used in reference to anti-CD30L antibody or antigen binding fragment means non-aggregated anti-CD30L antibody or antigen binding fragment. For example, in the context of a full-length anti-CD30L antibody, a monomer full length anti-CD30L antibody would mean a divalent anti-CD30L antibody molecule of about 150,000 Dalton with two heavy chain and two light chain, wherein the anti-CD30L antibody has not associated with, oligomerized with, or aggregated with any other antibody molecules. In one embodiment, the formulated antibodies provided herein remain substantially in a monomer form over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In one embodiment, the formulated antibodies provided herein remain substantially in a monomer form over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In another embodiment, the formulated antibodies provided herein remain substantially in a monomer form over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In another embodiment, the formulated antibodies provided herein remain substantially in a monomer form over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months 24 months or longer at 40±5° C. In one embodiment, the formulated antibodies provided herein remain substantially in a monomer form for at least 2 weeks when stored at 5±3° C. In one embodiment, the formulated antibodies provided herein remain substantially in a monomer form for at least 4 weeks when stored at 5±3° C. In one embodiment, the formulated antibodies provided herein remain substantially in a monomer form for at least 8 weeks when stored at 5±3° C. In one embodiment, the formulated antibodies provided herein remain substantially in a monomer form for at least 2 weeks when stored in the presence of 0.1-5.0 ppm H2O2 at 25±5° C. In one embodiment, the formulated antibodies provided herein remain substantially in a monomer form for at least 4 weeks when stored in the presence of 0.1-5.0 ppm H2O2 at 25±5° C. In one embodiment, the formulated antibodies provided herein remain substantially in a monomer form for at least 8 weeks when stored in the presence of 0.1-5.0 ppm H2O2 at 25±5° C. In one embodiment, the formulated antibodies provided herein remain substantially in a monomer form for at least 2 weeks when stored at 25±5° C. In one embodiment, the formulated antibodies provided herein remain substantially in a monomer form for at least 4 weeks when stored at 25±5° C. In one embodiment, the formulated antibodies provided herein remain substantially in a monomer form for at least 8 weeks when stored at 25±5° C. In one embodiment, the formulated antibodies provided herein remain substantially in a monomer form for at least 2 weeks when stored at 40±5° C. In one embodiment, the formulated antibodies provided herein remain substantially in a monomer form for at least 4 weeks when stored at 40±5° C. In one embodiment, the formulated antibodies provided herein remain substantially in a monomer form for at least 8 weeks when stored at 40±5° C.

In one embodiment, the formulated antibodies provided herein remain substantially in a monomer form over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In one embodiment, the monomer form of the antibody amounts to 50-60% (w/w), 60-70% (w/w), 70-80% (w/w), 80-85% (w/w), 85-90% (w/w), 90-95% (w/w), or 95-100% (w/w) of total protein in the pharmaceutical composition when stored at 5±3° C. In one embodiment, the monomer form of the antibody amounts to more than about 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), or 99.9% (w/w) of total protein in the pharmaceutical composition when stored at 5±3° C. In one embodiment, the formulated antibodies provided herein remain substantially in a monomer form over 2 weeks at 5±3° C., and the monomer form of the antibody is more than 90% (w/w), 91% (w/w), 92% (w/w), 93% (w/w), 94% (w/w), 95% (w/w), 96% (w/w), 97% (w/w), 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), or 99.9% (w/w) of total protein in the pharmaceutical composition at the end of the 2 week. In one embodiment, the formulated antibodies provided herein remain substantially in a monomer form over 4 weeks at 5±3° C., and the monomer form of the antibody is more than 90% (w/w), 91% (w/w), 92% (w/w), 93% (w/w), 94% (w/w), 95% (w/w), 96% (w/w), 97% (w/w), 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), or 99.9% (w/w) of total protein in the pharmaceutical composition at the end of the 4 week. In one embodiment, the formulated antibodies provided herein remain substantially in a monomer form over 8 weeks at 5±3° C., and the monomer form of the antibody is more than 90% (w/w), 91% (w/w), 92% (w/w), 93% (w/w), 94% (w/w), 95% (w/w), 96% (w/w), 97% (w/w), 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), or 99.9% (w/w) of total protein in the pharmaceutical composition at the end of the 8 week. In one embodiment, the formulated antibodies provided herein remain substantially in a monomer form over 8 weeks at 5±3° C., and the monomer form of the antibody is more than 90% (w/w), 91% (w/w), 92% (w/w), 93% (w/w), 94% (w/w), 95% (w/w), 96% (w/w), 97% (w/w), 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), or 99.9% (w/w) of total protein in the pharmaceutical composition at the end of the 8 week. In one embodiment, the formulated antibodies provided herein remain substantially in a monomer form over 12 weeks at 5±3° C., and the monomer form of the antibody is more than 90% (w/w), 91% (w/w), 92% (w/w), 93% (w/w), 94% (w/w), 95% (w/w), 96% (w/w), 97% (w/w), 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), or 99.9% (w/w) of total protein in the pharmaceutical composition at the end of the 12 week. In one embodiment, the formulated antibodies provided herein remain substantially in a monomer form over 26 weeks at 5±3° C., and the monomer form of the antibody is more than 90% (w/w), 91% (w/w), 92% (w/w), 93% (w/w), 94% (w/w), 95% (w/w), 96% (w/w), 97% (w/w), 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), or 99.9% (w/w) of total protein in the pharmaceutical composition at the end of the 26 week. In one embodiment, the formulated antibodies provided herein remain substantially in a monomer form over 2 weeks at 5±3° C., and the monomer form of the antibody is from 99% to 99.5% (w/w) of total protein in the pharmaceutical composition at the end of the 2 week. In one embodiment, the formulated antibodies provided herein remain substantially in a monomer form over 4 weeks at 5±3° C., and the monomer form of the antibody is from 99% to 99.5% (w/w) of total protein in the pharmaceutical composition at the end of the 4 week. In one embodiment, the formulated antibodies provided herein remain substantially in a monomer form over 8 weeks at 5±3° C., and the monomer form of the antibody is from 99% to 99.5% (w/w) of total protein in the pharmaceutical composition at the end of the 8 week. In one embodiment, the formulated antibodies provided herein remain substantially in a monomer form over 12 weeks at 5±3° C., and the monomer form of the antibody is from 99% to 99.5% (w/w) of total protein in the pharmaceutical composition at the end of the 12 week. In one embodiment, the formulated antibodies provided herein remain substantially in a monomer form over 26 weeks at 5±3° C., and the monomer form of the antibody is from 99% to 99.5% (w/w) of total protein in the pharmaceutical composition at the end of the 26 week.

In one embodiment, the formulated antibodies provided herein remain substantially in a monomer form over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In one embodiment, the monomer form of the antibody amounts to 50-60% (w/w), 60-70% (w/w), 70-80% (w/w), 80-85% (w/w), 85-90% (w/w), 90-95% (w/w), or 95-100% (w/w) of total protein in the pharmaceutical composition when stored in the presence of 0.1-5.0 ppm H2O2 at 25±5° C. In one embodiment, the monomer form of the antibody amounts to more than about 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), or 99.9% (w/w) of total protein in the pharmaceutical composition when stored in the presence of 0.1-5.0 ppm H2O2 at 25±5° C. In one embodiment, the formulated antibodies provided herein remain substantially in a monomer form over 2 weeks in the presence of 0.1-5.0 ppm H2O2 at 25±5° C., and the monomer form of the antibody is more than 90% (w/w), 91% (w/w), 92% (w/w), 93% (w/w), 94% (w/w), 95% (w/w), 96% (w/w), 97% (w/w), 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), or 99.9% (w/w) of total protein in the pharmaceutical composition at the end of the 2 week. In one embodiment, the formulated antibodies provided herein remain substantially in a monomer form over 4 weeks in the presence of 0.1-5.0 ppm H2O2 at 25±5° C., and the monomer form of the antibody is more than 90% (w/w), 91% (w/w), 92% (w/w), 93% (w/w), 94% (w/w), 95% (w/w), 96% (w/w), 97% (w/w), 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), or 99.9% (w/w) of total protein in the pharmaceutical composition at the end of the 4 week. In one embodiment, the formulated antibodies provided herein remain substantially in a monomer form over 8 weeks in the presence of 0.1-5.0 ppm H2O2 at 25±5° C., and the monomer form of the antibody is more than 90% (w/w), 91% (w/w), 92% (w/w), 93% (w/w), 94% (w/w), 95% (w/w), 96% (w/w), 97% (w/w), 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), or 99.9% (w/w) of total protein in the pharmaceutical composition at the end of the 8 week. In one embodiment, the formulated antibodies provided herein remain substantially in a monomer form over 8 weeks in the presence of 0.1-5.0 ppm H2O2 at 25±5° C., and the monomer form of the antibody is more than 90% (w/w), 91% (w/w), 92% (w/w), 93% (w/w), 94% (w/w), 95% (w/w), 96% (w/w), 97% (w/w), 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), or 99.9% (w/w) of total protein in the pharmaceutical composition at the end of the 8 week. In one embodiment, the formulated antibodies provided herein remain substantially in a monomer form over 12 weeks in the presence of 0.1-5.0 ppm H2O2 at 25±5° C., and the monomer form of the antibody is more than 90% (w/w), 91% (w/w), 92% (w/w), 93% (w/w), 94% (w/w), 95% (w/w), 96% (w/w), 97% (w/w), 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), or 99.9% (w/w) of total protein in the pharmaceutical composition at the end of the 12 week. In one embodiment, the formulated antibodies provided herein remain substantially in a monomer form over 26 weeks in the presence of 0.1-5.0 ppm H2O2 at 25±5° C., and the monomer form of the antibody is more than 90% (w/w), 91% (w/w), 92% (w/w), 93% (w/w), 94% (w/w), 95% (w/w), 96% (w/w), 97% (w/w), 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), or 99.9% (w/w) of total protein in the pharmaceutical composition at the end of the 26 week. In one embodiment, the formulated antibodies provided herein remain substantially in a monomer form over 2 weeks in the presence of 0.1-5.0 ppm H2O2 at 25±5° C., and the monomer form of the antibody is from 99% to 99.1% (w/w) of total protein in the pharmaceutical composition at the end of the 2 week. In one embodiment, the formulated antibodies provided herein remain substantially in a monomer form over 4 weeks in the presence of 0.1-5.0 ppm H2O2 at 25±5° C., and the monomer form of the antibody is from 99% to 99.1% (w/w) of total protein in the pharmaceutical composition at the end of the 4 week. In one embodiment, the formulated antibodies provided herein remain substantially in a monomer form over 8 weeks in the presence of 0.1-5.0 ppm H2O2 at 25±5° C., and the monomer form of the antibody is from 99% to 99.1% (w/w) of total protein in the pharmaceutical composition at the end of the 8 week. In one embodiment, the formulated antibodies provided herein remain substantially in a monomer form over 12 weeks in the presence of 0.1-5.0 ppm H2O2 at 25±5° C., and the monomer form of the antibody is from 99% to 99.1% (w/w) of total protein in the pharmaceutical composition at the end of the 12 week. In one embodiment, the formulated antibodies provided herein remain substantially in a monomer form over 26 weeks in the presence of 0.1-5.0 ppm H2O2 at 25±5° C., and the monomer form of the antibody is from 99% to 99.1% (w/w) of total protein in the pharmaceutical composition at the end of the 26 week.

In one embodiment, the formulated antibodies provided herein remain substantially in a monomer form over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In one embodiment, the monomer form of the antibody amounts to 50-60% (w/w), 60-70% (w/w), 70-80% (w/w), 80-85% (w/w), 85-90% (w/w), 90-95% (w/w), or 95-100% (w/w) of total protein in the pharmaceutical compositions when stored at 25±5° C. In one embodiment, the monomer form of the antibody amounts to more than about 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), 98.1% (w/w), 98.2% (w/w), 98.3% (w/w), 98.4% (w/w), 98.5% (w/w), 98.6% (w/w), 98.7% (w/w), 98.8% (w/w), 98.9% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), or 99.9% (w/w) of total protein in the pharmaceutical composition when stored at 25±5° C. In one embodiment, the formulated antibodies provided herein remain substantially in a monomer form over 2 weeks at 25±5° C., and the monomer form of the antibody is more than 90% (w/w), 91% (w/w), 92% (w/w), 93% (w/w), 94% (w/w), 95% (w/w), 96% (w/w), 97% (w/w), 98% (w/w), 98.1% (w/w), 98.2% (w/w), 98.3% (w/w), 98.4% (w/w), 98.5% (w/w), 98.6% (w/w), 98.7% (w/w), 98.8% (w/w), 98.9% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), or 99.9% (w/w) of total protein in the pharmaceutical composition at the end of the 2 week. In one embodiment, the formulated antibodies provided herein remain substantially in a monomer form over 4 weeks at 25±5° C., and the monomer form of the antibody is more than 90% (w/w), 91% (w/w), 92% (w/w), 93% (w/w), 94% (w/w), 95% (w/w), 96% (w/w), 97% (w/w), 98% (w/w), 98.1% (w/w), 98.2% (w/w), 98.3% (w/w), 98.4% (w/w), 98.5% (w/w), 98.6% (w/w), 98.7% (w/w), 98.8% (w/w), 98.9% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), or 99.9% (w/w) of total protein in the pharmaceutical composition at the end of the 4 week. In one embodiment, the formulated antibodies provided herein remain substantially in a monomer form over 8 weeks at 25±5° C., and the monomer form of the antibody is more than 90% (w/w), 91% (w/w), 92% (w/w), 93% (w/w), 94% (w/w), 95% (w/w), 96% (w/w), 97% (w/w), 98% (w/w), 98.1% (w/w), 98.2% (w/w), 98.3% (w/w), 98.4% (w/w), 98.5% (w/w), 98.6% (w/w), 98.7% (w/w), 98.8% (w/w), 98.9% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), or 99.9% (w/w) of total protein in the pharmaceutical composition at the end of the 8 week. In one embodiment, the formulated antibodies provided herein remain substantially in a monomer form over 8 weeks at 25±5° C., and the monomer form of the antibody is more than 90% (w/w), 91% (w/w), 92% (w/w), 93% (w/w), 94% (w/w), 95% (w/w), 96% (w/w), 97% (w/w), 98% (w/w), 98.1% (w/w), 98.2% (w/w), 98.3% (w/w), 98.4% (w/w), 98.5% (w/w), 98.6% (w/w), 98.7% (w/w), 98.8% (w/w), 98.9% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), or 99.9% (w/w) of total protein in the pharmaceutical composition at the end of the 8 week. In one embodiment, the formulated antibodies provided herein remain substantially in a monomer form over 12 weeks at 25±5° C., and the monomer form of the antibody is more than 90% (w/w), 91% (w/w), 92% (w/w), 93% (w/w), 94% (w/w), 95% (w/w), 96% (w/w), 97% (w/w), 98% (w/w), 98.1% (w/w), 98.2% (w/w), 98.3% (w/w), 98.4% (w/w), 98.5% (w/w), 98.6% (w/w), 98.7% (w/w), 98.8% (w/w), 98.9% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), or 99.9% (w/w) of total protein in the pharmaceutical composition at the end of the 12 week. In one embodiment, the formulated antibodies provided herein remain substantially in a monomer form over 26 weeks at 25±5° C., and the monomer form of the antibody is more than 90% (w/w), 91% (w/w), 92% (w/w), 93% (w/w), 94% (w/w), 95% (w/w), 96% (w/w), 97% (w/w), 98% (w/w), 98.1% (w/w), 98.2% (w/w), 98.3% (w/w), 98.4% (w/w), 98.5% (w/w), 98.6% (w/w), 98.7% (w/w), 98.8% (w/w), 98.9% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), or 99.9% (w/w) of total protein in the pharmaceutical composition at the end of the 26 week. In one embodiment, the formulated antibodies provided herein remain substantially in a monomer form over 2 weeks at 25±5° C., and the monomer form of the antibody is from 98% to 99% (w/w) of total protein in the pharmaceutical composition at the end of the 2 week. In one embodiment, the formulated antibodies provided herein remain substantially in a monomer form over 4 weeks at 25±5° C., and the monomer form of the antibody is from 98% to 99% (w/w) of total protein in the pharmaceutical composition at the end of the 4 week. In one embodiment, the formulated antibodies provided herein remain substantially in a monomer form over 8 weeks at 25±5° C., and the monomer form of the antibody is from 98% to 99% (w/w) of total protein in the pharmaceutical composition at the end of the 8 week. In one embodiment, the formulated antibodies provided herein remain substantially in a monomer form over 12 weeks at 25±5° C., and the monomer form of the antibody is from 98% to 99% (w/w) of total protein in the pharmaceutical composition at the end of the 12 week. In one embodiment, the formulated antibodies provided herein remain substantially in a monomer form over 26 weeks at 25±5° C., and the monomer form of the antibody is from 98% to 99% (w/w) of total protein in the pharmaceutical composition at the end of the 26 week.

In one embodiment, the formulated antibodies provided herein remain substantially in a monomer form over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40±5° C. In one embodiment, the monomer form of the antibody amounts to 50-60% (w/w), 60-70% (w/w), 70-80% (w/w), 80-85% (w/w), 85-90% (w/w), 90-95% (w/w), or 95-100% (w/w) of total protein in the pharmaceutical compositions when stored at 40±5° C. In one embodiment, the monomer form of the antibody amounts to more than about 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 97.1% (w/w), 97.2% (w/w), 97.3% (w/w), 97.4% (w/w), 97.5% (w/w), 97.6% (w/w), 97.7% (w/w), 97.8% (w/w), 97.9% (w/w), 98% (w/w), 98.1% (w/w), 98.2% (w/w), 98.3% (w/w), 98.4% (w/w), 98.5% (w/w), 98.6% (w/w), 98.7% (w/w), 98.8% (w/w), 98.9% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), or 99.9% (w/w) of total protein in the pharmaceutical composition when stored at 40±5° C. In one embodiment, the formulated antibodies provided herein remain substantially in a monomer form over 2 weeks at 40±5° C., and the monomer form of the antibody is more than 90% (w/w), 91% (w/w), 92% (w/w), 93% (w/w), 94% (w/w), 95% (w/w), 96% (w/w), 97% (w/w), 97.1% (w/w), 97.2% (w/w), 97.3% (w/w), 97.4% (w/w), 97.5% (w/w), 97.6% (w/w), 97.7% (w/w), 97.8% (w/w), 97.9% (w/w), 98% (w/w), 98.1% (w/w), 98.2% (w/w), 98.3% (w/w), 98.4% (w/w), 98.5% (w/w), 98.6% (w/w), 98.7% (w/w), 98.8% (w/w), 98.9% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), or 99.9% (w/w) of total protein in the pharmaceutical composition at the end of the 2 week. In one embodiment, the formulated antibodies provided herein remain substantially in a monomer form over 4 weeks at 40±5° C., and the monomer form of the antibody is more than 90% (w/w), 91% (w/w), 92% (w/w), 93% (w/w), 94% (w/w), 95% (w/w), 96% (w/w), 97% (w/w), 97.1% (w/w), 97.2% (w/w), 97.3% (w/w), 97.4% (w/w), 97.5% (w/w), 97.6% (w/w), 97.7% (w/w), 97.8% (w/w), 97.9% (w/w), 98% (w/w), 98.1% (w/w), 98.2% (w/w), 98.3% (w/w), 98.4% (w/w), 98.5% (w/w), 98.6% (w/w), 98.7% (w/w), 98.8% (w/w), 98.9% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), or 99.9% (w/w) of total protein in the pharmaceutical composition at the end of the 4 week. In one embodiment, the formulated antibodies provided herein remain substantially in a monomer form over 8 weeks at 40±5° C., and the monomer form of the antibody is more than 90% (w/w), 91% (w/w), 92% (w/w), 93% (w/w), 94% (w/w), 95% (w/w), 96% (w/w), 97% (w/w), 97.1% (w/w), 97.2% (w/w), 97.3% (w/w), 97.4% (w/w), 97.5% (w/w), 97.6% (w/w), 97.7% (w/w), 97.8% (w/w), 97.9% (w/w), 98% (w/w), 98.1% (w/w), 98.2% (w/w), 98.3% (w/w), 98.4% (w/w), 98.5% (w/w), 98.6% (w/w), 98.7% (w/w), 98.8% (w/w), 98.9% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), or 99.9% (w/w) of total protein in the pharmaceutical composition at the end of the 8 week. In one embodiment, the formulated antibodies provided herein remain substantially in a monomer form over 8 weeks at 40±5° C., and the monomer form of the antibody is more than 90% (w/w), 91% (w/w), 92% (w/w), 93% (w/w), 94% (w/w), 95% (w/w), 96% (w/w), 97% (w/w), 97.1% (w/w), 97.2% (w/w), 97.3% (w/w), 97.4% (w/w), 97.5% (w/w), 97.6% (w/w), 97.7% (w/w), 97.8% (w/w), 97.9% (w/w), 98% (w/w), 98.1% (w/w), 98.2% (w/w), 98.3% (w/w), 98.4% (w/w), 98.5% (w/w), 98.6% (w/w), 98.7% (w/w), 98.8% (w/w), 98.9% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), or 99.9% (w/w) of total protein in the pharmaceutical composition at the end of the 8 week. In one embodiment, the formulated antibodies provided herein remain substantially in a monomer form over 12 weeks at 40±5° C., and the monomer form of the antibody is more than 90% (w/w), 91% (w/w), 92% (w/w), 93% (w/w), 94% (w/w), 95% (w/w), 96% (w/w), 97% (w/w), 97.1% (w/w), 97.2% (w/w), 97.3% (w/w), 97.4% (w/w), 97.5% (w/w), 97.6% (w/w), 97.7% (w/w), 97.8% (w/w), 97.9% (w/w), 98% (w/w), 98.1% (w/w), 98.2% (w/w), 98.3% (w/w), 98.4% (w/w), 98.5% (w/w), 98.6% (w/w), 98.7% (w/w), 98.8% (w/w), 98.9% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), or 99.9% (w/w) of total protein in the pharmaceutical composition at the end of the 12 week. In one embodiment, the formulated antibodies provided herein remain substantially in a monomer form over 26 weeks at 40±5° C., and the monomer form of the antibody is more than 90% (w/w), 91% (w/w), 92% (w/w), 93% (w/w), 94% (w/w), 95% (w/w), 96% (w/w), 97% (w/w), 97.1% (w/w), 97.2% (w/w), 97.3% (w/w), 97.4% (w/w), 97.5% (w/w), 97.6% (w/w), 97.7% (w/w), 97.8% (w/w), 97.9% (w/w), 98% (w/w), 98.1% (w/w), 98.2% (w/w), 98.3% (w/w), 98.4% (w/w), 98.5% (w/w), 98.6% (w/w), 98.7% (w/w), 98.8% (w/w), 98.9% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), or 99.9% (w/w) of total protein in the pharmaceutical composition at the end of the 26 week. In one embodiment, the formulated antibodies provided herein remain substantially in a monomer form over 2 weeks at 40±5° C., and the monomer form of the antibody is from 97% to 97.9% (w/w) of total protein in the pharmaceutical composition at the end of the 2 week. In one embodiment, the formulated antibodies provided herein remain substantially in a monomer form over 4 weeks at 40±5° C., and the monomer form of the antibody is from 97% to 97.9% (w/w) of total protein in the pharmaceutical composition at the end of the 4 week. In one embodiment, the formulated antibodies provided herein remain substantially in a monomer form over 8 weeks at 40±5° C., and the monomer form of the antibody is from 90% to 97.9% (w/w) of total protein in the pharmaceutical composition at the end of the 8 week. In one embodiment, the formulated antibodies provided herein remain substantially in a monomer form over 12 weeks at 40±5° C., and the monomer form of the antibody is from 90% to 97.9% (w/w) of total protein in the pharmaceutical composition at the end of the 12 week. In one embodiment, the formulated antibodies provided herein remain substantially in a monomer form over 26 weeks at 40±5° C., and the monomer form of the antibody is from 90% to 97.9% (w/w) of total protein in the pharmaceutical composition at the end of the 26 week.

In one embodiment, the formulated antibodies provided herein remain substantially in a monomer form after agitation (e.g. 100 RPM at room temperature) for up to 3 days, agitation (e.g. 100 RPM at room temperature) for up to 5 days, or vigorous agitation (e.g. 300 RPM at room temperature) for up to 3 days. In one embodiment, the monomer form of the antibody amounts to 50-60% (w/w), 60-70% (w/w), 70-80% (w/w), 80-85% (w/w), 85-90% (w/w), 90-95% (w/w), or 95-100% (w/w) of total protein in the pharmaceutical compositions after agitation (e.g. 100 RPM at room temperature) for up to 3 days, agitation (e.g. 100 RPM at room temperature) for up to 5 days, or vigorous agitation (e.g. 300 RPM at room temperature) for up to 3 days. In one embodiment, formulated antibodies provided herein remain substantially in a monomer form after agitation (e.g. 100 RPM at room temperature) for up to 3 days, agitation (e.g. 100 RPM at room temperature) for up to 5 days, or vigorous agitation (e.g. 300 RPM at room temperature) for up to 3 days, and the monomer form of the antibody is more than 90% (w/w), 91% (w/w), 92% (w/w), 93% (w/w), 94% (w/w), 95% (w/w), 96% (w/w), 97% (w/w), 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), or 99.9% (w/w) of total protein in the pharmaceutical composition at the end of such agitation. In one embodiment, the formulated antibodies provided herein remain substantially in a monomer form after agitation (e.g. 100 RPM at room temperature) for up to 3 days, agitation (e.g. 100 RPM at room temperature) for up to 5 days, or vigorous agitation (e.g. 300 RPM at room temperature) for up to 3 days, and the monomer form of the antibody is from 99% to 99.5% (w/w) of total protein in the pharmaceutical composition at the end of such agitation. In one embodiment, the formulated antibodies provided herein remain substantially in a monomer form after agitation (e.g. 100 RPM at room temperature) for up to 3 days, agitation (e.g. 100 RPM at room temperature) for up to 5 days, or vigorous agitation (e.g. 300 RPM at room temperature) for up to 3 days, and the monomer form of the antibody is from 99% to 99.3% (w/w) of total protein in the pharmaceutical composition at the end of such agitation.

In a specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 2 weeks storage at 5±3° C., the monomer form of the antibody that binds to CD30L is from 99% to 99.5% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 4 weeks storage at 5±3° C., the monomer form of the antibody that binds to CD30L is from 99% to 99.5% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 8 weeks storage at 5±3° C., the monomer form of the antibody that binds to CD30L is from 99% to 99.5% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 12 weeks storage at 5±3° C., the monomer form of the antibody that binds to CD30L is from 99% to 99.5% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 26 weeks storage at 5±3° C., the monomer form of the antibody that binds to CD30L is from 99% to 99.5% (w/w) of total protein in the pharmaceutical composition. In a specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 2 weeks storage at 5±3° C., the monomer form of the antibody that binds to CD30L is from 99% to 99.5% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 4 weeks storage at 5±3° C., the monomer form of the antibody that binds to CD30L is from 99% to 99.5% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 8 weeks storage at 5±3° C., the monomer form of the antibody that binds to CD30L is from 99% to 99.5% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 12 weeks storage at 5±3° C., the monomer form of the antibody that binds to CD30L is from 99% to 99.5% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 26 weeks storage at 5±3° C., the monomer form of the antibody that binds to CD30L is from 99% to 99.5% (w/w) of total protein in the pharmaceutical composition.

In a specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 2 weeks storage in the presence of 0.1-5.0 ppm H2O2 at 25±5° C., the monomer form of the antibody that binds to CD30L is no less than 99% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 4 weeks storage in the presence of 0.1-5.0 ppm H2O2 at 25±5° C., the monomer form of the antibody that binds to CD30L is no less than 99% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 8 weeks storage in the presence of 0.1-5.0 ppm H2O2 at 25±5° C., the monomer form of the antibody that binds to CD30L is no less than 99% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 12 weeks storage in the presence of 0.1-5.0 ppm H2O2 at 25±5° C., the monomer form of the antibody that binds to CD30L is no less than 99% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 26 weeks storage in the presence of 0.1-5.0 ppm H2O2 at 25±5° C., the monomer form of the antibody that binds to CD30L is no less than 99% (w/w) of total protein in the pharmaceutical composition. In a specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 2 weeks storage in the presence of 0.1-5.0 ppm H2O2 at 25±5° C., the monomer form of the antibody that binds to CD30L is no less than 99% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 4 weeks storage in the presence of 0.1-5.0 ppm H2O2 at 25±5° C., the monomer form of the antibody that binds to CD30L is no less than 99% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 8 weeks storage in the presence of 0.1-5.0 ppm H2O2 at 25±5° C., the monomer form of the antibody that binds to CD30L is no less than 99% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 12 weeks storage in the presence of 0.1-5.0 ppm H2O2 at 25±5° C., the monomer form of the antibody that binds to CD30L is no less than 99% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 26 weeks storage in the presence of 0.1-5.0 ppm H2O2 at 25±5° C., the monomer form of the antibody that binds to CD30L is no less than 99% (w/w) of total protein in the pharmaceutical composition.

In a specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 2 weeks storage at 25±5° C., the monomer form of the antibody that binds to CD30L is from 98% to 99% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 4 weeks storage at 25±5° C., the monomer form of the antibody that binds to CD30L is from 98% to 99% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 8 weeks storage at 25±5° C., the monomer form of the antibody that binds to CD30L is from 98% to 99% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 12 weeks storage at 25±5° C., the monomer form of the antibody that binds to CD30L is from 98% to 99% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 26 weeks storage at 25±5° C., the monomer form of the antibody that binds to CD30L is from 98% to 99% (w/w) of total protein in the pharmaceutical composition. In a specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 2 weeks storage at 25±5° C., the monomer form of the antibody that binds to CD30L is from 98% to 99% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 4 weeks storage at 25±5° C., the monomer form of the antibody that binds to CD30L is from 98% to 99% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 8 weeks storage at 25±5° C., the monomer form of the antibody that binds to CD30L is from 98% to 99% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 12 weeks storage at 25±5° C., the monomer form of the antibody that binds to CD30L is from 98% to 99% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 26 weeks storage at 25±5° C., the monomer form of the antibody that binds to CD30L is from 98% to 99% (w/w) of total protein in the pharmaceutical composition.

In a specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 2 weeks storage at 40±5° C., the monomer form of the antibody that binds to CD30L is from 97% to 97.9% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 4 weeks storage at 40±5° C., the monomer form of the antibody that binds to CD30L is from 96% to 97.4% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 8 weeks storage at 40±5° C., the monomer form of the antibody that binds to CD30L is from 95% to 97.9% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 12 weeks storage at 40±5° C., the monomer form of the antibody that binds to CD30L is from 90% to 97.9% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 26 weeks storage at 40±5° C., the monomer form of the antibody that binds to CD30L is from 90% to 97.9% (w/w) of total protein in the pharmaceutical composition. In a specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 2 weeks storage at 40±5° C., the monomer form of the antibody that binds to CD30L is from 97% to 97.9% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 4 weeks storage at 40±5° C., the monomer form of the antibody that binds to CD30L is from 96% to 97.9% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 8 weeks storage at 40±5° C., the monomer form of the antibody that binds to CD30L is from 90% to 97.9% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 12 weeks storage at 40±5° C., the monomer form of the antibody that binds to CD30L is from 90% to 97.9% (w/w) of total protein in the pharmaceutical composition. In another specific embodiment, provided herein is a pharmaceutical composition comprising an antibody or antigen-binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 26 weeks storage at 40±5° C., the monomer form of the antibody that binds to CD30L is from 90% to 97.9% (w/w) of total protein in the pharmaceutical composition.

In one embodiment, the formulated antibodies provided herein form no more than a trace amount of aggregated high molecular weight (“HMW”) species over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In another embodiment, the formulated antibodies provided herein form no more than a trace amount of aggregated HMW species over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In another embodiment, the formulated antibodies provided herein form no more than a trace amount of aggregated HMW species over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months 24 months or longer at 40±5° C.

In one embodiment, the formulated antibodies provided herein form no more than a trace amount of aggregated HMW species over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. Trace amount when used in reference to the HMW species can be determined by a percentage of the HMW species in the total weight of the antibody or antigen binding fragment in the pharmaceutical composition as provided herein, for example, in this and the following paragraphs. In one embodiment, the HMW species of the antibody amounts to less than 10% (w/w), 9% (w/w), 8% (w/w), 7% (w/w), 6% (w/w), 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions. In one embodiment, the HMW species of the antibody amounts to 0-0.05% (w/w), 0.05-0.1% (w/w), 0.1-0.5% (w/w), 0.5-1% (w/w), 1-1.5% (w/w), 1.5-2% (w/w), 2-2.5% (w/w), 2.5-3% (w/w), 3-3.5% (w/w), 3.5-4% (w/w), 4-4.5% (w/w), 4.5-5% (w/w), 5-5.5% (w/w), 5.5-6% (w/w), 6-6.5% (w/w), 6.5-7% (w/w), 7-7.5% (w/w), 7.5-8% (w/w), 8-8.5% (w/w), 8.5-9% (w/w), 9-9.5% (w/w), 9.5-10% (w/w). In one embodiment, the formulated antibodies provided herein form no more than a trace amount of aggregated HMW species over 2 weeks at 5±3° C., and the trace amount of HMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 2 weeks. In one embodiment, the formulated antibodies provided herein form no more than a trace amount of aggregated HMW species over 2 weeks at 5±3° C., and the trace amount of HMW species of the antibody is from 0.1% to 1% (w/w) of total protein in the pharmaceutical composition at the end of the 2 weeks. In one embodiment, the formulated antibodies provided herein form no more than a trace amount of aggregated HMW species over 2 weeks at 5±3° C., and the trace amount of HMW species of the antibody is from 0.5% to 0.7% (w/w) of total protein in the pharmaceutical composition at the end of the 2 weeks. In one embodiment, the formulated antibodies provided herein form no more than a trace amount of aggregated HMW species over 4 weeks at 5±3° C., and the trace amount of HMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 4 weeks. In one embodiment, the formulated antibodies provided herein form no more than a trace amount of aggregated HMW species over 4 weeks at 5±3° C., and the trace amount of HMW species of the antibody is from 0.1% to 1% (w/w) of total protein in the pharmaceutical composition at the end of the 4 weeks. In one embodiment, the formulated antibodies provided herein form no more than a trace amount of aggregated HMW species over 4 weeks at 5±3° C., and the trace amount of HMW species of the antibody is from 0.5% to 0.7% (w/w) of total protein in the pharmaceutical composition at the end of the 4 weeks. In one embodiment, the formulated antibodies provided herein form no more than a trace amount of aggregated HMW species over 8 weeks at 5±3° C., and the trace amount of HMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 8 weeks. In one embodiment, the formulated antibodies provided herein form no more than a trace amount of aggregated HMW species over 8 weeks at 5±3° C., and the trace amount of HMW species of the antibody is from 0.1% to 1% (w/w) of total protein in the pharmaceutical composition at the end of the 8 weeks. In one embodiment, the formulated antibodies provided herein form no more than a trace amount of aggregated HMW species over 8 weeks at 5±3° C., and the trace amount of HMW species of the antibody is from 0.5% to 0.7% (w/w) of total protein in the pharmaceutical composition at the end of the 8 weeks. In one embodiment, the formulated antibodies provided herein form no more than a trace amount of aggregated HMW species over 12 weeks at 5±3° C., and the trace amount of HMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 12 weeks. In one embodiment, the formulated antibodies provided herein form no more than a trace amount of aggregated HMW species over 12 weeks at 5±3° C., and the trace amount of HMW species of the antibody is from 0.1% to 1% (w/w) of total protein in the pharmaceutical composition at the end of the 12 weeks. In one embodiment, the formulated antibodies provided herein form no more than a trace amount of aggregated HMW species over 12 weeks at 5±3° C., and the trace amount of HMW species of the antibody is from 0.5% to 0.7% (w/w) of total protein in the pharmaceutical composition at the end of the 12 weeks. In one embodiment, the formulated antibodies provided herein form no more than a trace amount of aggregated HMW species over 6 months at 5±3° C., and the trace amount of HMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 6 months. In one embodiment, the formulated antibodies provided herein form no more than a trace amount of aggregated HMW species over 6 months at 5±3° C., and the trace amount of HMW species of the antibody is from 0.1% to 1% (w/w) of total protein in the pharmaceutical composition at the end of the 6 months. In one embodiment, the formulated antibodies provided herein form no more than a trace amount of aggregated HMW species over 6 months at 5±3° C., and the trace amount of HMW species of the antibody is from 0.5% to 0.7% (w/w) of total protein in the pharmaceutical composition at the end of the 6 months.

In one embodiment, the formulated antibodies provided herein form no more than a trace amount of aggregated HMW species over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. Trace amount when used in reference to the HMW species can be determined by a percentage of the HMW species in the total weight of the antibody or antigen binding fragment in the pharmaceutical composition as provided herein, for example, in this and the following paragraphs. In one embodiment, the HMW species of the antibody amounts to less than 10% (w/w), 9% (w/w), 8% (w/w), 7% (w/w), 6% (w/w), 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions. In one embodiment, the HMW species of the antibody amounts to 0-0.05% (w/w), 0.05-0.1% (w/w), 0.1-0.5% (w/w), 0.5-1% (w/w), 1-1.5% (w/w), 1.5-2% (w/w), 2-2.5% (w/w), 2.5-3% (w/w), 3-3.5% (w/w), 3.5-4% (w/w), 4-4.5% (w/w), 4.5-5% (w/w), 5-5.5% (w/w), 5.5-6% (w/w), 6-6.5% (w/w), 6.5-7% (w/w), 7-7.5% (w/w), 7.5-8% (w/w), 8-8.5% (w/w), 8.5-9% (w/w), 9-9.5% (w/w), 9.5-10% (w/w). In one embodiment, the formulated antibodies provided herein form no more than a trace amount of aggregated HMW species over 2 weeks in the presence of 0.1-5.0 ppm H2O2 at 25±5° C., and the trace amount of HMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 2 weeks. In one embodiment, the formulated antibodies provided herein form no more than a trace amount of aggregated HMW species over 2 weeks in the presence of 0.1-5.0 ppm H2O2 at 25±5° C., and the trace amount of HMW species of the antibody is from 0.1% to 1% (w/w) of total protein in the pharmaceutical composition at the end of the 2 weeks. In one embodiment, the formulated antibodies provided herein form no more than a trace amount of aggregated HMW species over 2 weeks in the presence of 0.1-5.0 ppm H2O2 at 25±5° C., and the trace amount of HMW species of the antibody is no more than 0.7% (w/w) of total protein in the pharmaceutical composition at the end of the 2 weeks. In one embodiment, the formulated antibodies provided herein form no more than a trace amount of aggregated HMW species over 4 weeks in the presence of 0.1-5.0 ppm H2O2 at 25±5° C., and the trace amount of HMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 4 weeks. In one embodiment, the formulated antibodies provided herein form no more than a trace amount of aggregated HMW species over 4 weeks in the presence of 0.1-5.0 ppm H2O2 at 25±5° C., and the trace amount of HMW species of the antibody is from 0.1% to 1% (w/w) of total protein in the pharmaceutical composition at the end of the 4 weeks. In one embodiment, the formulated antibodies provided herein form no more than a trace amount of aggregated HMW species over 4 weeks in the presence of 0.1-5.0 ppm H2O2 at 25±5° C., and the trace amount of HMW species of the antibody is no more than 0.9% (w/w) of total protein in the pharmaceutical composition at the end of the 4 weeks. In one embodiment, the formulated antibodies provided herein form no more than a trace amount of aggregated HMW species over 8 weeks in the presence of 0.1-5.0 ppm H2O2 at 25±5° C., and the trace amount of HMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 8 weeks. In one embodiment, the formulated antibodies provided herein form no more than a trace amount of aggregated HMW species over 8 weeks in the presence of 0.1-5.0 ppm H2O2 at 25±5° C., and the trace amount of HMW species of the antibody is from 0.1% to 1% (w/w) of total protein in the pharmaceutical composition at the end of the 8 weeks. In one embodiment, the formulated antibodies provided herein form no more than a trace amount of aggregated HMW species over 8 weeks in the presence of 0.1-5.0 ppm H2O2 at 25±5° C., and the trace amount of HMW species of the antibody is no more than 0.9% (w/w) of total protein in the pharmaceutical composition at the end of the 8 weeks. In one embodiment, the formulated antibodies provided herein form no more than a trace amount of aggregated HMW species over 12 weeks in the presence of 0.1-5.0 ppm H2O2 at 25±5° C., and the trace amount of HMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 12 weeks. In one embodiment, the formulated antibodies provided herein form no more than a trace amount of aggregated HMW species over 12 weeks in the presence of 0.1-5.0 ppm H2O2 at 25±5° C., and the trace amount of HMW species of the antibody is from 0.1% to 1% (w/w) of total protein in the pharmaceutical composition at the end of the 12 weeks. In one embodiment, the formulated antibodies provided herein form no more than a trace amount of aggregated HMW species over 12 weeks in the presence of 0.1-5.0 ppm H2O2 at 25±5° C., and the trace amount of HMW species of the antibody is no more than 0.9% (w/w) of total protein in the pharmaceutical composition at the end of the 12 weeks. In one embodiment, the formulated antibodies provided herein form no more than a trace amount of aggregated HMW species over 6 months in the presence of 0.1-5.0 ppm H2O2 at 25±5° C., and the trace amount of HMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 6 months. In one embodiment, the formulated antibodies provided herein form no more than a trace amount of aggregated HMW species over 6 months in the presence of 0.1-5.0 ppm H2O2 at 25±5° C., and the trace amount of HMW species of the antibody is from 0.1% to 1% (w/w) of total protein in the pharmaceutical composition at the end of the 6 months. In one embodiment, the formulated antibodies provided herein form no more than a trace amount of aggregated HMW species over 6 months in the presence of 0.1-5.0 ppm H2O2 at 25±5° C., and the trace amount of HMW species of the antibody is no more than 0.9% (w/w) of total protein in the pharmaceutical composition at the end of the 6 months.

In one embodiment, the formulated antibodies provided herein form no more than a trace amount of aggregated HMW species over 2 weeks at 25±5° C., and the trace amount of HMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 2 weeks. In one embodiment, the formulated antibodies provided herein form no more than a trace amount of aggregated HMW species over 2 weeks at 25±5° C., and the trace amount of HMW species of the antibody is from 0.1% to 1% (w/w) of total protein in the pharmaceutical composition at the end of the 2 weeks. In one embodiment, the formulated antibodies provided herein form no more than a trace amount of aggregated HMW species over 2 weeks at 25±5° C., and the trace amount of HMW species of the antibody is from 0.5% to 0.7% (w/w) of total protein in the pharmaceutical composition at the end of the 2 weeks. In one embodiment, the formulated antibodies provided herein form no more than a trace amount of aggregated HMW species over 4 weeks at 25±5° C., and the trace amount of HMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 4 weeks. In one embodiment, the formulated antibodies provided herein form no more than a trace amount of aggregated HMW species over 4 weeks at 25±5° C., and the trace amount of HMW species of the antibody is from 0.1% to 1% (w/w) of total protein in the pharmaceutical composition at the end of the 4 weeks. In one embodiment, the formulated antibodies provided herein form no more than a trace amount of aggregated HMW species over 4 weeks at 25±5° C., and the trace amount of HMW species of the antibody is from 0.7% to 0.9% (w/w) of total protein in the pharmaceutical composition at the end of the 4 weeks. In one embodiment, the formulated antibodies provided herein form no more than a trace amount of aggregated HMW species over 8 weeks at 25±5° C., and the trace amount of HMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 8 weeks. In one embodiment, the formulated antibodies provided herein form no more than a trace amount of aggregated HMW species over 8 weeks at 25±5° C., and the trace amount of HMW species of the antibody is from 0.1% to 1.1% (w/w) of total protein in the pharmaceutical composition at the end of the 8 weeks. In one embodiment, the formulated antibodies provided herein form no more than a trace amount of aggregated HMW species over 8 weeks at 25±5° C., and the trace amount of HMW species of the antibody is from 0.8% to 1.1% (w/w) of total protein in the pharmaceutical composition at the end of the 8 weeks. In one embodiment, the formulated antibodies provided herein form no more than a trace amount of aggregated HMW species over 12 weeks at 25±5° C., and the trace amount of HMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 12 weeks. In one embodiment, the formulated antibodies provided herein form no more than a trace amount of aggregated HMW species over 12 weeks at 25±5° C., and the trace amount of HMW species of the antibody is from 0.1% to 1.1% (w/w) of total protein in the pharmaceutical composition at the end of the 12 weeks. In one embodiment, the formulated antibodies provided herein form no more than a trace amount of aggregated HMW species over 12 weeks at 25±5° C., and the trace amount of HMW species of the antibody is from 0.8% to 1.1% (w/w) of total protein in the pharmaceutical composition at the end of the 12 weeks. In one embodiment, the formulated antibodies provided herein form no more than a trace amount of aggregated HMW species over 6 months at 25±5° C., and the trace amount of HMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 6 months. In one embodiment, the formulated antibodies provided herein form no more than a trace amount of aggregated HMW species over 6 months at 25±5° C., and the trace amount of HMW species of the antibody is from 0.1% to 2% (w/w) of total protein in the pharmaceutical composition at the end of the 6 months. In one embodiment, the formulated antibodies provided herein form no more than a trace amount of aggregated HMW species over 6 months at 25±5° C., and the trace amount of HMW species of the antibody is from 0.5% to 2.0% (w/w) of total protein in the pharmaceutical composition at the end of the 6 months.

In one embodiment, the formulated antibodies provided herein form no more than a trace amount of aggregated HMW species over 2 weeks at 40±5° C., and the trace amount of HMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 2 weeks. In one embodiment, the formulated antibodies provided herein form no more than a trace amount of aggregated HMW species over 2 weeks at 40±5° C., and the trace amount of HMW species of the antibody is from 0.1% to 2% (w/w) of total protein in the pharmaceutical composition at the end of the 2 weeks. In one embodiment, the formulated antibodies provided herein form no more than a trace amount of aggregated HMW species over 2 weeks at 40±5° C., and the trace amount of HMW species of the antibody is from 1.0% to 1.5% (w/w) of total protein in the pharmaceutical composition at the end of the 2 weeks. In one embodiment, the formulated antibodies provided herein form no more than a trace amount of aggregated HMW species over 4 weeks at 40±5° C., and the trace amount of HMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 4 weeks. In one embodiment, the formulated antibodies provided herein form no more than a trace amount of aggregated HMW species over 4 weeks at 40±5° C., and the trace amount of HMW species of the antibody is from 0.1% to 3% (w/w) of total protein in the pharmaceutical composition at the end of the 4 weeks. In one embodiment, the formulated antibodies provided herein form no more than a trace amount of aggregated HMW species over 4 weeks at 40±5° C., and the trace amount of HMW species of the antibody is from 1.5% to 2.0% (w/w) of total protein in the pharmaceutical composition at the end of the 4 weeks. In one embodiment, the formulated antibodies provided herein form no more than a trace amount of aggregated HMW species over 8 weeks at 40±5° C., and the trace amount of HMW species of the antibody is less than 15%, 10%, 9%, 8%, 7%, 6%, 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 8 weeks. In one embodiment, the formulated antibodies provided herein form no more than a trace amount of aggregated HMW species over 8 weeks at 40±5° C., and the trace amount of HMW species of the antibody is from 0.1% to 5% (w/w) of total protein in the pharmaceutical composition at the end of the 8 weeks. In one embodiment, the formulated antibodies provided herein form no more than a trace amount of aggregated HMW species over 8 weeks at 40±5° C., and the trace amount of HMW species of the antibody is from 1.0% to 5.0% (w/w) of total protein in the pharmaceutical composition at the end of the 8 weeks. In one embodiment, the formulated antibodies provided herein form no more than a trace amount of aggregated HMW species over 12 weeks at 40±5° C., and the trace amount of HMW species of the antibody is less than 20%, 15%, 10%, 9%, 8%, 7%, 6%, 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 12 weeks. In one embodiment, the formulated antibodies provided herein form no more than a trace amount of aggregated HMW species over 12 weeks at 40±5° C., and the trace amount of HMW species of the antibody is from 1% to 10% (w/w) of total protein in the pharmaceutical composition at the end of the 12 weeks. In one embodiment, the formulated antibodies provided herein form no more than a trace amount of aggregated HMW species over 12 weeks at 40±5° C., and the trace amount of HMW species of the antibody is from 3% to 10% (w/w) of total protein in the pharmaceutical composition at the end of the 12 weeks. In one embodiment, the formulated antibodies provided herein form no more than a trace amount of aggregated HMW species over 6 months at 40±5° C., and the trace amount of HMW species of the antibody is less than 30%, 25%, 20%, 15%, 10%, 9%, 8%, 7%, 6%, 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 6 months. In one embodiment, the formulated antibodies provided herein form no more than a trace amount of aggregated HMW species over 6 months at 40±5° C., and the trace amount of HMW species of the antibody is from 1% to 15% (w/w) of total protein in the pharmaceutical composition at the end of the 6 months. In one embodiment, the formulated antibodies provided herein form no more than a trace amount of aggregated HMW species over 6 months at 40±5° C., and the trace amount of HMW species of the antibody is from 5% to 15% (w/w) of total protein in the pharmaceutical composition at the end of the 6 months.

In one embodiment, the formulated antibodies provided herein form no more than a trace amount of aggregated HMW species after agitation (e.g. 100 RPM at room temperature) for up to 3 days, agitation (e.g. 100 RPM at room temperature) for up to 5 days, or vigorous agitation (e.g. 300 RPM at room temperature) for up to 3 days. In one embodiment, the HMW species of the antibody amounts to less than 10% (w/w), 9% (w/w), 8% (w/w), 7% (w/w), 6% (w/w), 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions after agitation (e.g. 100 RPM at room temperature) for up to 3 days, agitation (e.g. 100 RPM at room temperature) for up to 5 days, or vigorous agitation (e.g. 300 RPM at room temperature) for up to 3 days. In one embodiment, the HMW species of the antibody amounts to 0-0.05% (w/w), 0.05-0.1% (w/w), 0.1-0.5% (w/w), 0.5-1% (w/w), 1-1.5% (w/w), 1.5-2% (w/w), 2-2.5% (w/w), 2.5-3% (w/w), 3-3.5% (w/w), 3.5-4% (w/w), 4-4.5% (w/w), 4.5-5% (w/w), 5-5.5% (w/w), 5.5-6% (w/w), 6-6.5% (w/w), 6.5-7% (w/w), 7-7.5% (w/w), 7.5-8% (w/w), 8-8.5% (w/w), 8.5-9% (w/w), 9-9.5% (w/w), 9.5-10% (w/w) after agitation (e.g. 100 RPM at room temperature) for up to 3 days, agitation (e.g. 100 RPM at room temperature) for up to 5 days, or vigorous agitation (e.g. 300 RPM at room temperature) for up to 3 days. In one embodiment, the formulated antibodies provided herein form no more than a trace amount of aggregated HMW species after agitation (e.g. 100 RPM at room temperature) for up to 3 days, agitation (e.g. 100 RPM at room temperature) for up to 5 days, or vigorous agitation (e.g. 300 RPM at room temperature) for up to 3 days, and the trace amount of HMW species of the antibody is from 0.1% to 1% (w/w) of total protein in the pharmaceutical composition at the end of the agitation. In one embodiment, the formulated antibodies provided herein form no more than a trace amount of aggregated HMW species after agitation (e.g. 100 RPM at room temperature) for up to 3 days, agitation (e.g. 100 RPM at room temperature) for up to 5 days, or vigorous agitation (e.g. 300 RPM at room temperature) for up to 3 days, and the trace amount of HMW species of the antibody is from 0.4% to 0.5% (w/w) of total protein in the pharmaceutical composition at the end of the agitation.

In one embodiment, the formulated antibodies provided herein form no more than a trace amount of aggregated HMW species after up to 3 cycles, up to 5 cycles, or up to 10 cycles of freeze and thaw from −80° C. to room temperature. In one embodiment, the HMW species of the antibody amounts to less than 10% (w/w), 9% (w/w), 8% (w/w), 7% (w/w), 6% (w/w), 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions after up to 3 cycles, up to 5 cycles, or up to 10 cycles of freeze and thaw from −80° C. to room temperature. In one embodiment, the HMW species of the antibody amounts to 0-0.05% (w/w), 0.05-0.1% (w/w), 0.1-0.5% (w/w), 0.5-1% (w/w), 1-1.5% (w/w), 1.5-2% (w/w), 2-2.5% (w/w), 2.5-3% (w/w), 3-3.5% (w/w), 3.5-4% (w/w), 4-4.5% (w/w), 4.5-5% (w/w), 5-5.5% (w/w), 5.5-6% (w/w), 6-6.5% (w/w), 6.5-7% (w/w), 7-7.5% (w/w), 7.5-8% (w/w), 8-8.5% (w/w), 8.5-9% (w/w), 9-9.5% (w/w), 9.5-10% (w/w) after up to 3 cycles, up to 5 cycles, or up to 10 cycles of freeze and thaw from −80° C. to room temperature. In one embodiment, the formulated antibodies provided herein form no more than a trace amount of aggregated HMW species after up to 3 cycles, up to 5 cycles, or up to 10 cycles of freeze and thaw from −80° C. to room temperature, and the trace amount of HMW species of the antibody is from 0.1% to 1% (w/w) of total protein in the pharmaceutical composition at the end of the freeze and thaw cycle. In one embodiment, the formulated antibodies provided herein form no more than a trace amount of aggregated HMW species after up to 3 cycles, up to 5 cycles, or up to 10 cycles of freeze and thaw from −80° C. to room temperature, and the trace amount of HMW species of the antibody is from 0.4% to 0.7% (w/w) of total protein in the pharmaceutical composition at the end of the freeze and thaw cycle.

In a specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 2 or 4 weeks storage at 5° C., the HMW species of the antibody that binds to CD30L is no more than 0.5% (w/w) of total protein in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 12 weeks storage at 5° C., the HMW species of the antibody that binds to CD30L is no more than 0.6% (w/w) of total protein in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 2 weeks storage at 25° C., the HMW species of the antibody that binds to CD30L is no more than 0.6% (w/w) of total protein in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 4 weeks storage at 25° C., the HMW species of the antibody that binds to CD30L is no more than 0.8% (w/w) of total protein in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 12 weeks storage at 25° C., the HMW species of the antibody that binds to CD30L is no more than 1.1% (w/w) of total protein in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 2 weeks storage at 40° C., the HMW species of the antibody that binds to CD30L is no more than 1.2% (w/w) of total protein in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 4 weeks storage at 40° C., the HMW species of the antibody that binds to CD30L is no more than 1.8% (w/w) of total protein in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after up to 3 cycles, up to 5 cycles, or up to 10 cycles of freeze and thaw from −80° C. to room temperature, the HMW species of the antibody that binds to CD30L is less than 0.7% (w/w) of total protein in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after agitation (e.g. 100 RPM at room temperature) for up to 3 days, agitation (e.g. 100 RPM at room temperature) for up to 5 days, or vigorous agitation (e.g. 300 RPM at room temperature) for up to 3 days, the HMW species of the antibody that binds to CD30L is no more than 0.5% (w/w) of total protein in the pharmaceutical composition. In a specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 2 weeks storage in the presence of 0.1-5.0 ppm H2O2 at 25±5° C., the HMW species of the antibody that binds to CD30L is no more than 0.7% (w/w) of total protein in the pharmaceutical composition. In one specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 4 weeks storage in the presence of 0.1-5.0 ppm H2O2 at 25±5° C., the HMW species of the antibody that binds to CD30L is no more than 0.9% (w/w) of total protein in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 12 weeks storage in the presence of 0.1-5.0 ppm H2O2 at 25±5° C., the HMW species of the antibody that binds to CD30L is no more than 0.9% (w/w) of total protein in the pharmaceutical composition.

In a specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 2 or 4 weeks storage at 5° C., the HMW species of the antibody that binds to CD30L is no more than 0.5% (w/w) of total protein in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 12 weeks storage at 5° C., the HMW species of the antibody that binds to CD30L is no more than 0.6% (w/w) of total protein in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 2 weeks storage at 25° C., the HMW species of the antibody that binds to CD30L is no more than 0.6% (w/w) of total protein in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 4 weeks storage at 25° C., the HMW species of the antibody that binds to CD30L is no more than 0.8% (w/w) of total protein in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 12 weeks storage at 25° C., the HMW species of the antibody that binds to CD30L is no more than 1.1% (w/w) of total protein in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 2 weeks storage at 40° C., the HMW species of the antibody that binds to CD30L is no more than 1.2% (w/w) of total protein in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 4 weeks storage at 40° C., the HMW species of the antibody that binds to CD30L is no more than 1.8% (w/w) of total protein in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after up to 3 cycles, up to 5 cycles, or up to 10 cycles of freeze and thaw from −80° C. to room temperature, the HMW species of the antibody that binds to CD30L is less than 0.7% (w/w) of total protein in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after agitation (e.g. 100 RPM at room temperature) for up to 3 days, agitation (e.g. 100 RPM at room temperature) for up to 5 days, or vigorous agitation (e.g. 300 RPM at room temperature) for up to 3 days, the HMW species of the antibody that binds to CD30L is no more than 0.5% (w/w) of total protein in the pharmaceutical composition. In a specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 2 weeks storage in the presence of 0.1-5.0 ppm H2O2 at 25±5° C., the HMW species of the antibody that binds to CD30L is no more than 0.7% (w/w) of total protein in the pharmaceutical composition. In one specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 4 weeks storage in the presence of 0.1-5.0 ppm H2O2 at 25±5° C., the HMW species of the antibody that binds to CD30L is no more than 0.9% (w/w) of total protein in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 12 weeks storage in the presence of 0.1-5.0 ppm H2O2 at 25±5° C., the HMW species of the antibody that binds to CD30L is no more than 0.9% (w/w) of total protein in the pharmaceutical composition.

In one embodiment, the formulated antibodies provided herein form no more than a trace amount of fragmented (clipped) low molecular weight (“LMW”) species over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In one embodiment, the formulated antibodies provided herein form no more than a trace amount of fragmented (clipped) LMW species over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In one embodiment, the formulated antibodies provided herein form no more than a trace amount of fragmented (clipped) LMW species over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40±5° C.

In one embodiment, the formulated antibodies provided herein form no more than a trace amount of fragmented (clipped) LMW species over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5° C. In one embodiment, the LMW species of the antibody amounts to less than 10% (w/w), 9% (w/w), 8% (w/w), 7% (w/w), 6% (w/w), 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions after storage at 5° C. for various durations. In one embodiment, the LMW species of the antibody amounts to 0.01-0.05% (w/w), 0.05-0.1% (w/w), 0.1-0.5% (w/w), 0.5-1% (w/w), 1-1.5% (w/w), 1.5-2% (w/w), 2-2.5% (w/w), 2.5-3% (w/w), 3-3.5% (w/w), 3.5-4% (w/w), 4-4.5% (w/w), 4.5-5% (w/w), 5-5.5% (w/w), 5.5-6% (w/w), 6-6.5% (w/w), 6.5-7% (w/w), 7-7.5% (w/w), 7.5-8% (w/w), 8-8.5% (w/w), 8.5-9% (w/w), 9-9.5% (w/w), 9.5-10% (w/w) after storage at 5° C. for various durations. In one embodiment, the formulated antibodies provided herein form no more than a trace amount of fragmented (clipped) LMW species over 2 weeks at 5° C., and the trace amount of LMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 2 weeks. In one embodiment, the formulated antibodies provided herein form no more than a trace amount of fragmented (clipped) LMW species over 2 weeks at 5° C., and the trace amount of LMW species of the antibody is from 0.01% to 0.1% (w/w) of total protein in the pharmaceutical composition at the end of the 2 weeks. In one embodiment, the formulated antibodies provided herein form no more than a trace amount of fragmented (clipped) LMW species over 2 weeks at 5° C., and the trace amount of LMW species of the antibody is below detection limit of total protein in the pharmaceutical composition at the end of the 2 weeks. In one embodiment, the formulated antibodies provided herein form no more than a trace amount of fragmented (clipped) LMW species over 4 weeks at 5° C., and the trace amount of LMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 4 weeks. In one embodiment, the formulated antibodies provided herein form no more than a trace amount of fragmented (clipped) LMW species over 4 weeks at 5° C., and the trace amount of LMW species of the antibody is from 0.01% to 0.1% (w/w) of total protein in the pharmaceutical composition at the end of the 4 weeks. In one embodiment, the formulated antibodies provided herein form no more than a trace amount of fragmented (clipped) LMW species over 4 weeks at 5° C., and the trace amount of LMW species of the antibody is below detection limit of total protein in the pharmaceutical composition at the end of the 4 weeks. In one embodiment, the formulated antibodies provided herein form no more than a trace amount of fragmented (clipped) LMW species over 8 weeks at 5° C., and the trace amount of LMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 8 weeks. In one embodiment, the formulated antibodies provided herein form no more than a trace amount of fragmented (clipped) LMW species over 8 weeks at 5° C., and the trace amount of LMW species of the antibody is from 0.01% to 0.1% (w/w) of total protein in the pharmaceutical composition at the end of the 8 weeks. In one embodiment, the formulated antibodies provided herein form no more than a trace amount of fragmented (clipped) LMW species over 8 weeks at 5° C., and the trace amount of LMW species of the antibody is below detection limit of total protein in the pharmaceutical composition at the end of the 8 weeks. In one embodiment, the formulated antibodies provided herein form no more than a trace amount of fragmented (clipped) LMW species over 12 weeks at 5° C., and the trace amount of LMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 12 weeks. In one embodiment, the formulated antibodies provided herein form no more than a trace amount of fragmented (clipped) LMW species over 12 weeks at 5° C., and the trace amount of LMW species of the antibody is from 0.01% to 0.1% (w/w) of total protein in the pharmaceutical composition at the end of the 12 weeks. In one embodiment, the formulated antibodies provided herein form no more than a trace amount of fragmented (clipped) LMW species over 12 weeks at 5° C., and the trace amount of LMW species of the antibody is below detection limit of total protein in the pharmaceutical composition at the end of the 12 weeks. In one embodiment, the formulated antibodies provided herein form no more than a trace amount of fragmented (clipped) LMW species over 6 months at 5° C., and the trace amount of LMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 6 months. In one embodiment, the formulated antibodies provided herein form no more than a trace amount of fragmented (clipped) LMW species over 6 months at 5° C., and the trace amount of LMW species of the antibody is from 0.01% to 0.5% (w/w) of total protein in the pharmaceutical composition at the end of the 6 months. In one embodiment, the formulated antibodies provided herein form no more than a trace amount of fragmented (clipped) LMW species over 6 months at 5° C., and the trace amount of LMW species of the antibody is from 0.01% to 3% (w/w) of total protein in the pharmaceutical composition at the end of the 6 months. In some embodiment, the detection limit referenced in this paragraph is set by SE-UPLC assay.

In one embodiment, the formulated antibodies provided herein form no more than a trace amount of fragmented (clipped) LMW species over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25° C. In one embodiment, the LMW species of the antibody amounts to less than 10% (w/w), 9% (w/w), 8% (w/w), 7% (w/w), 6% (w/w), 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions after storage at 25° C. for various durations. In one embodiment, the LMW species of the antibody amounts to 0.01-0.05% (w/w), 0.05-0.1% (w/w), 0.1-0.5% (w/w), 0.5-1% (w/w), 1-1.5% (w/w), 1.5-2% (w/w), 2-2.5% (w/w), 2.5-3% (w/w), 3-3.5% (w/w), 3.5-4% (w/w), 4-4.5% (w/w), 4.5-5% (w/w), 5-5.5% (w/w), 5.5-6% (w/w), 6-6.5% (w/w), 6.5-7% (w/w), 7-7.5% (w/w), 7.5-8% (w/w), 8-8.5% (w/w), 8.5-9% (w/w), 9-9.5% (w/w), 9.5-10% (w/w) after storage at 25° C. for various durations. In one embodiment, the formulated antibodies provided herein form no more than a trace amount of fragmented (clipped) LMW species over 2 weeks at 25° C., and the trace amount of LMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 2 weeks. In one embodiment, the formulated antibodies provided herein form no more than a trace amount of fragmented (clipped) LMW species over 2 weeks at 25° C., and the trace amount of LMW species of the antibody is from 0.01% to 0.3% (w/w) of total protein in the pharmaceutical composition at the end of the 2 weeks. In one embodiment, the formulated antibodies provided herein form no more than a trace amount of fragmented (clipped) LMW species over 2 weeks at 25° C., and the trace amount of LMW species of the antibody is no more than 0.3% (w/w) of total protein in the pharmaceutical composition at the end of the 2 weeks. In one embodiment, the formulated antibodies provided herein form no more than a trace amount of fragmented (clipped) LMW species over 4 weeks at 25° C., and the trace amount of LMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 4 weeks. In one embodiment, the formulated antibodies provided herein form no more than a trace amount of fragmented (clipped) LMW species over 4 weeks at 25° C., and the trace amount of LMW species of the antibody is from 0.01% to 0.3% (w/w) of total protein in the pharmaceutical composition at the end of the 4 weeks. In one embodiment, the formulated antibodies provided herein form no more than a trace amount of fragmented (clipped) LMW species over 4 weeks at 25° C., and the trace amount of LMW species of the antibody is no more than 0.3% (w/w) of total protein in the pharmaceutical composition at the end of the 4 weeks. In one embodiment, the formulated antibodies provided herein form no more than a trace amount of fragmented (clipped) LMW species over 8 weeks at 25° C., and the trace amount of LMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 8 weeks. In one embodiment, the formulated antibodies provided herein form no more than a trace amount of fragmented (clipped) LMW species over 8 weeks at 25° C., and the trace amount of LMW species of the antibody is from 0.01% to 0.4% (w/w) of total protein in the pharmaceutical composition at the end of the 8 weeks. In one embodiment, the formulated antibodies provided herein form no more than a trace amount of fragmented (clipped) LMW species over 8 weeks at 25° C., and the trace amount of LMW species of the antibody is from no more than 0.4% (w/w) of total protein in the pharmaceutical composition at the end of the 8 weeks. In one embodiment, the formulated antibodies provided herein form no more than a trace amount of fragmented (clipped) LMW species over 12 weeks at 25° C., and the trace amount of LMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 12 weeks. In one embodiment, the formulated antibodies provided herein form no more than a trace amount of fragmented (clipped) LMW species over 12 weeks at 25° C., and the trace amount of LMW species of the antibody is from 0.01% to 1.0% (w/w) of total protein in the pharmaceutical composition at the end of the 12 weeks. In one embodiment, the formulated antibodies provided herein form no more than a trace amount of fragmented (clipped) LMW species over 12 weeks at 25° C., and the trace amount of LMW species of the antibody is from 0.1% to 0.5% (w/w) of total protein in the pharmaceutical composition at the end of the 12 weeks. In one embodiment, the formulated antibodies provided herein form no more than a trace amount of fragmented (clipped) LMW species over 6 months at 25° C., and the trace amount of LMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 6 months. In one embodiment, the formulated antibodies provided herein form no more than a trace amount of fragmented (clipped) LMW species over 6 months at 25° C., and the trace amount of LMW species of the antibody is from 0.1% to 3.0% (w/w) of total protein in the pharmaceutical composition at the end of the 6 months. In one embodiment, the formulated antibodies provided herein form no more than a trace amount of fragmented (clipped) LMW species over 6 months at 25° C., and the trace amount of LMW species of the antibody is from 0.1% to 1% (w/w) of total protein in the pharmaceutical composition at the end of the 6 months.

In one embodiment, the formulated antibodies provided herein form no more than a trace amount of fragmented (clipped) LMW species over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40° C. In one embodiment, the LMW species of the antibody amounts to less than 10% (w/w), 9% (w/w), 8% (w/w), 7% (w/w), 6% (w/w), 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions. In one embodiment, the LMW species of the antibody amounts to 0.01-0.05% (w/w), 0.05-0.1% (w/w), 0.1-0.5% (w/w), 0.5-1% (w/w), 1-1.5% (w/w), 1.5-2% (w/w), 2-2.5% (w/w), 2.5-3% (w/w), 3-3.5% (w/w), 3.5-4% (w/w), 4-4.5% (w/w), 4.5-5% (w/w), 5-5.5% (w/w), 5.5-6% (w/w), 6-6.5% (w/w), 6.5-7% (w/w), 7-7.5% (w/w), 7.5-8% (w/w), 8-8.5% (w/w), 8.5-9% (w/w), 9-9.5% (w/w), 9.5-10% (w/w). In one embodiment, the formulated antibodies provided herein form no more than a trace amount of fragmented (clipped) LMW species over 2 weeks at 40° C., and the trace amount of LMW species of the antibody is less than 10% (w/w), 9% (w/w), 8% (w/w), 7% (w/w), 6% (w/w), 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 2 weeks. In one embodiment, the formulated antibodies provided herein form no more than a trace amount of fragmented (clipped) LMW species over 2 weeks at 40° C., and the trace amount of LMW species of the antibody is from 0.01% to 2% (w/w) of total protein in the pharmaceutical composition at the end of the 2 weeks. In one embodiment, the formulated antibodies provided herein form no more than a trace amount of fragmented (clipped) LMW species over 2 weeks at 40° C., and the trace amount of LMW species of the antibody is from 0.1% to 1% (w/w) of total protein in the pharmaceutical composition at the end of the 2 weeks. In one embodiment, the formulated antibodies provided herein form no more than a trace amount of fragmented (clipped) LMW species over 4 weeks at 40° C., and the trace amount of LMW species of the antibody is less than 10% (w/w), 9% (w/w), 8% (w/w), 7% (w/w), 6% (w/w), 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 4 weeks. In one embodiment, the formulated antibodies provided herein form no more than a trace amount of fragmented (clipped) LMW species over 4 weeks at 40° C., and the trace amount of LMW species of the antibody is from 0.1% to 2% (w/w) of total protein in the pharmaceutical composition at the end of the 4 weeks. In one embodiment, the formulated antibodies provided herein form no more than a trace amount of fragmented (clipped) LMW species over 4 weeks at 40° C., and the trace amount of LMW species of the antibody is from 0.1% to 1% (w/w) of total protein in the pharmaceutical composition at the end of the 4 weeks. In one embodiment, the formulated antibodies provided herein form no more than a trace amount of fragmented (clipped) LMW species over 8 weeks at 40° C., and the trace amount of LMW species of the antibody is less than 10% (w/w), 9% (w/w), 8% (w/w), 7% (w/w), 6% (w/w), 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 8 weeks. In one embodiment, the formulated antibodies provided herein form no more than a trace amount of fragmented (clipped) LMW species over 8 weeks at 40° C., and the trace amount of LMW species of the antibody is from 0.1% to 5% (w/w) of total protein in the pharmaceutical composition at the end of the 8 weeks. In one embodiment, the formulated antibodies provided herein form no more than a trace amount of fragmented (clipped) LMW species over 8 weeks at 40° C., and the trace amount of LMW species of the antibody is from 1% to 5% (w/w) of total protein in the pharmaceutical composition at the end of the 8 weeks. In one embodiment, the formulated antibodies provided herein form no more than a trace amount of fragmented (clipped) LMW species over 12 weeks at 40° C., and the trace amount of LMW species of the antibody is less than 10% (w/w), 9% (w/w), 8% (w/w), 7% (w/w), 6% (w/w), 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 12 weeks. In one embodiment, the formulated antibodies provided herein form no more than a trace amount of fragmented (clipped) LMW species over 12 weeks at 40° C., and the trace amount of LMW species of the antibody is from 0.1% to 10% (w/w) of total protein in the pharmaceutical composition at the end of the 12 weeks. In one embodiment, the formulated antibodies provided herein form no more than a trace amount of fragmented (clipped) LMW species over 12 weeks at 40° C., and the trace amount of LMW species of the antibody is from 1% to 10% (w/w) of total protein in the pharmaceutical composition at the end of the 12 weeks. In one embodiment, the formulated antibodies provided herein form no more than a trace amount of fragmented (clipped) LMW species over 6 months at 40° C., and the trace amount of LMW species of the antibody is less than 15% (w/w), 14% (w/w), 13% (w/w), 12% (w/w), 11% (w/w), 10% (w/w), 9% (w/w), 8% (w/w), 7% (w/w), 6% (w/w), 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 6 months. In one embodiment, the formulated antibodies provided herein form no more than a trace amount of fragmented (clipped) LMW species over 6 months at 40° C., and the trace amount of LMW species of the antibody is from 0.1% to 15% (w/w) of total protein in the pharmaceutical composition at the end of the 6 months. In one embodiment, the formulated antibodies provided herein form no more than a trace amount of fragmented (clipped) LMW species over 6 months at 40° C., and the trace amount of LMW species of the antibody is from 2% to 15% (w/w) of total protein in the pharmaceutical composition at the end of the 6 months.

In one embodiment, the formulated antibodies provided herein form no more than a trace amount of fragmented (clipped) LMW species over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In one embodiment, the LMW species of the antibody amounts to less than 10% (w/w), 9% (w/w), 8% (w/w), 7% (w/w), 6% (w/w), 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions after storage in the presence of 0.1-5.0 ppm H2O2 at 25±5° C. for various durations. In one embodiment, the LMW species of the antibody amounts to 0.01-0.05% (w/w), 0.05-0.1% (w/w), 0.1-0.5% (w/w), 0.5-1% (w/w), 1-1.5% (w/w), 1.5-2% (w/w), 2-2.5% (w/w), 2.5-3% (w/w), 3-3.5% (w/w), 3.5-4% (w/w), 4-4.5% (w/w), 4.5-5% (w/w), 5-5.5% (w/w), 5.5-6% (w/w), 6-6.5% (w/w), 6.5-7% (w/w), 7-7.5% (w/w), 7.5-8% (w/w), 8-8.5% (w/w), 8.5-9% (w/w), 9-9.5% (w/w), 9.5-10% (w/w) after storage in the presence of 0.1-5.0 ppm H2O2 at 25±5° C. for various durations. In one embodiment, the formulated antibodies provided herein form no more than a trace amount of fragmented (clipped) LMW species over 2 weeks in the presence of 0.1-5.0 ppm H2O2 at 25±5° C., and the trace amount of LMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 2 weeks. In one embodiment, the formulated antibodies provided herein form no more than a trace amount of fragmented (clipped) LMW species over 2 weeks in the presence of 0.1-5.0 ppm H2O2 at 25±5° C., and the trace amount of LMW species of the antibody is from 0.01% to 0.5% (w/w) of total protein in the pharmaceutical composition at the end of the 2 weeks. In one embodiment, the formulated antibodies provided herein form no more than a trace amount of fragmented (clipped) LMW species over 2 weeks in the presence of 0.1-5.0 ppm H2O2 at 25±5° C., and the trace amount of LMW species of the antibody is no more than 0.3% (w/w) of total protein in the pharmaceutical composition at the end of the 2 weeks. In one embodiment, the formulated antibodies provided herein form no more than a trace amount of fragmented (clipped) LMW species over 4 weeks in the presence of 0.1-5.0 ppm H2O2 at 25±5° C., and the trace amount of LMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 4 weeks. In one embodiment, the formulated antibodies provided herein form no more than a trace amount of fragmented (clipped) LMW species over 4 weeks in the presence of 0.1-5.0 ppm H2O2 at 25±5° C., and the trace amount of LMW species of the antibody is from 0.01% to 0.5% (w/w) of total protein in the pharmaceutical composition at the end of the 4 weeks. In one embodiment, the formulated antibodies provided herein form no more than a trace amount of fragmented (clipped) LMW species over 4 weeks in the presence of 0.1-5.0 ppm H2O2 at 25±5° C., and the trace amount of LMW species of the antibody is no more than 0.3% (w/w) of total protein in the pharmaceutical composition at the end of the 4 weeks. In one embodiment, the formulated antibodies provided herein form no more than a trace amount of fragmented (clipped) LMW species over 8 weeks in the presence of 0.1-5.0 ppm H2O2 at 25±5° C., and the trace amount of LMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 8 weeks. In one embodiment, the formulated antibodies provided herein form no more than a trace amount of fragmented (clipped) LMW species over 8 weeks in the presence of 0.1-5.0 ppm H2O2 at 25±5° C., and the trace amount of LMW species of the antibody is from 0.01% to 0.5% (w/w) of total protein in the pharmaceutical composition at the end of the 8 weeks. In one embodiment, the formulated antibodies provided herein form no more than a trace amount of fragmented (clipped) LMW species over 8 weeks in the presence of 0.1-5.0 ppm H2O2 at 25±5° C., and the trace amount of LMW species of the antibody is no more than 0.3% (w/w) of total protein in the pharmaceutical composition at the end of the 8 weeks. In one embodiment, the formulated antibodies provided herein form no more than a trace amount of fragmented (clipped) LMW species over 12 weeks in the presence of 0.1-5.0 ppm H2O2 at 25±5° C., and the trace amount of LMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 12 weeks. In one embodiment, the formulated antibodies provided herein form no more than a trace amount of fragmented (clipped) LMW species over 12 weeks in the presence of 0.1-5.0 ppm H2O2 at 25±5° C., and the trace amount of LMW species of the antibody is from 0.01% to 0.5% (w/w) of total protein in the pharmaceutical composition at the end of the 12 weeks. In one embodiment, the formulated antibodies provided herein form no more than a trace amount of fragmented (clipped) LMW species over 12 weeks in the presence of 0.1-5.0 ppm H2O2 at 25±5° C., and the trace amount of LMW species of the antibody is no more than 0.3% (w/w) of total protein in the pharmaceutical composition at the end of the 12 weeks. In one embodiment, the formulated antibodies provided herein form no more than a trace amount of fragmented (clipped) LMW species over 6 months in the presence of 0.1-5.0 ppm H2O2 at 25±5° C., and the trace amount of LMW species of the antibody is less than 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions at the end of the 6 months. In one embodiment, the formulated antibodies provided herein form no more than a trace amount of fragmented (clipped) LMW species over 6 months in the presence of 0.1-5.0 ppm H2O2 at 25±5° C., and the trace amount of LMW species of the antibody is from 0.01% to 0.5% (w/w) of total protein in the pharmaceutical composition at the end of the 6 months. In one embodiment, the formulated antibodies provided herein form no more than a trace amount of fragmented (clipped) LMW species over 6 months in the presence of 0.1-5.0 ppm H2O2 at 25±5° C., and the trace amount of LMW species of the antibody is from 0.01% to 3% (w/w) of total protein in the pharmaceutical composition at the end of the 6 months.

In one embodiment, the formulated antibodies provided herein form no more than a trace amount of fragmented (clipped) LMW species after agitation (e.g. 100 RPM at room temperature) for up to 3 days, agitation (e.g. 100 RPM at room temperature) for up to 5 days, or vigorous agitation (e.g. 300 RPM at room temperature) for up to 3 days. In one embodiment, the LMW species of the antibody amounts to less than 10% (w/w), 9% (w/w), 8% (w/w), 7% (w/w), 6% (w/w), 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions after agitation (e.g. 100 RPM at room temperature) for up to 3 days, agitation (e.g. 100 RPM at room temperature) for up to 5 days, or vigorous agitation (e.g. 300 RPM at room temperature) for up to 3 days. In one embodiment, the LMW species of the antibody amounts to 0.01-0.05% (w/w), 0.05-0.1% (w/w), 0.1-0.5% (w/w), 0.5-1% (w/w), 1-1.5% (w/w), 1.5-2% (w/w), 2-2.5% (w/w), 2.5-3% (w/w), 3-3.5% (w/w), 3.5-4% (w/w), 4-4.5% (w/w), 4.5-5% (w/w), 5-5.5% (w/w), 5.5-6% (w/w), 6-6.5% (w/w), 6.5-7% (w/w), 7-7.5% (w/w), 7.5-8% (w/w), 8-8.5% (w/w), 8.5-9% (w/w), 9-9.5% (w/w), 9.5-10% (w/w) after agitation (e.g. 100 RPM at room temperature) for up to 3 days, agitation (e.g. 100 RPM at room temperature) for up to 5 days, or vigorous agitation (e.g. 300 RPM at room temperature) for up to 3 days. In one embodiment, the formulated antibodies provided herein form no more than a trace amount of fragmented (clipped) LMW species after agitation (e.g. 100 RPM at room temperature) for up to 3 days, agitation (e.g. 100 RPM at room temperature) for up to 5 days, or vigorous agitation (e.g. 300 RPM at room temperature) for up to 3 days, and the trace amount of LMW species of the antibody is below detection limit in the pharmaceutical composition at the end of the agitation, wherein the detection limit is set by SE-UPLC assay. In one embodiment, the formulated antibodies provided herein form no more than a trace amount of fragmented (clipped) LMW species after agitation (e.g. 100 RPM at room temperature) for up to 3 days, agitation (e.g. 100 RPM at room temperature) for up to 5 days, or vigorous agitation (e.g. 300 RPM at room temperature) for up to 3 days, and the trace amount of LMW species of the antibody is from 0.01% to 0.1% (w/w) of total protein in the pharmaceutical composition at the end of the agitation.

In one embodiment, the formulated antibodies provided herein form no more than a trace amount of fragmented (clipped) LMW species after up to 3 cycles, up to 5 cycles, or up to 10 cycles of freeze and thaw from −80° C. to room temperature. In one embodiment, the LMW species of the antibody amounts to less than 10% (w/w), 9% (w/w), 8% (w/w), 7% (w/w), 6% (w/w), 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) of total protein in the pharmaceutical compositions after up to 3 cycles, up to 5 cycles, or up to 10 cycles of freeze and thaw from −80° C. to room temperature. In one embodiment, the LMW species of the antibody amounts to 0.01-0.05% (w/w), 0.05-0.1% (w/w), 0.1-0.5% (w/w), 0.5-1% (w/w), 1-1.5% (w/w), 1.5-2% (w/w), 2-2.5% (w/w), 2.5-3% (w/w), 3-3.5% (w/w), 3.5-4% (w/w), 4-4.5% (w/w), 4.5-5% (w/w), 5-5.5% (w/w), 5.5-6% (w/w), 6-6.5% (w/w), 6.5-7% (w/w), 7-7.5% (w/w), 7.5-8% (w/w), 8-8.5% (w/w), 8.5-9% (w/w), 9-9.5% (w/w), 9.5-10% (w/w) after up to 3 cycles, up to 5 cycles, or up to 10 cycles of freeze and thaw from −80° C. to room temperature. In one embodiment, the formulated antibodies provided herein form no more than a trace amount of fragmented (clipped) LMW species after up to 3 cycles, up to 5 cycles, or up to 10 cycles of freeze and thaw from −80° C. to room temperature, and the trace amount of LMW species of the antibody is from 0.01% to 1% (w/w) of total protein in the pharmaceutical composition at the end of the freeze and thaw cycles. In one embodiment, the formulated antibodies provided herein form no more than a trace amount of fragmented (clipped) LMW species after up to 3 cycles, up to 5 cycles, or up to 10 cycles of freeze and thaw from −80° C. to room temperature, and the trace amount of LMW species of the antibody is from 0.01% to 0.1% (w/w) of total protein in the pharmaceutical composition at the end of the freeze and thaw cycles.

In one specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 2 weeks storage at 5° C., the fragmented (clipped) LMW species of the antibody is no more than 0.1% (w/w) of total protein or below the detection limit in the pharmaceutical composition. In a specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 4 weeks storage at 5° C., the fragmented (clipped) LMW species of the antibody is no more than 0.1% (w/w) of total protein or below the detection limit in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 12 weeks storage at 5° C., the fragmented (clipped) LMW species of the antibody is no more than 0.1% (w/w) of total protein or below the detection limit in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 2 weeks storage at 25° C., the fragmented (clipped) LMW species of the antibody is no more than 0.3% (w/w) of total protein in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 4 weeks storage at 25° C., the fragmented (clipped) LMW species of the antibody is no more than 0.3% (w/w) of total protein in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 12 weeks storage at 25° C., the fragmented (clipped) LMW species of the antibody is no more than 0.4% (w/w) of total protein in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 2 weeks storage at 40° C., the fragmented (clipped) LMW species of the antibody is no more than 0.9% (w/w) of total protein in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 4 weeks storage at 40° C., the fragmented (clipped) LMW species of the antibody is no more than 0.9% (w/w) of total protein in the pharmaceutical composition. In one specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 2 weeks storage in the presence of 0.1-5.0 ppm H2O2 at 25±5° C., the fragmented (clipped) LMW species of the antibody is no more than 0.3% (w/w) of total protein or below the detection limit in the pharmaceutical composition. In a specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 4 weeks storage in the presence of 0.1-5.0 ppm H2O2 at 25±5° C., the fragmented (clipped) LMW species of the antibody is no more than 0.3% (w/w) of total protein or below the detection limit in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 12 weeks storage in the presence of 0.1-5.0 ppm H2O2 at 25±5° C., the fragmented (clipped) LMW species of the antibody is no more than 0.3% (w/w) of total protein or below the detection limit in the pharmaceutical composition.

In a specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 2 weeks storage at 5° C., the fragmented (clipped) LMW species of the antibody is no more than 0.1% (w/w) of total protein or below the detection limit in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 4 weeks storage at 5° C., the fragmented (clipped) LMW species of the antibody is no more than 0.1% (w/w) of total protein or below the detection limit in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 12 weeks storage at 5° C., the fragmented (clipped) LMW species of the antibody is no more than 0.1% (w/w) of total protein or below the detection limit in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 2 weeks storage at 25° C., the fragmented (clipped) LMW species of the antibody is no more than 0.3% (w/w) of total protein in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 4 weeks storage at 25° C., the fragmented (clipped) LMW species of the antibody is no more than 0.3% (w/w) of total protein in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 12 weeks storage at 25° C., the fragmented (clipped) LMW species of the antibody is no more than 0.4% (w/w) of total protein in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 2 weeks storage at 40° C., the fragmented (clipped) LMW species of the antibody is no more than 0.9% (w/w) of total protein in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 4 weeks storage at 40° C., the fragmented (clipped) LMW species of the antibody is no more than 0.9% (w/w) of total protein in the pharmaceutical composition. In a specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 2 weeks storage in the presence of 0.1-5.0 ppm H2O2 at 25±5° C., the fragmented (clipped) LMW species of the antibody is no more than 0.3% (w/w) of total protein or below the detection limit in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 4 weeks storage in the presence of 0.1-5.0 ppm H2O2 at 25±5° C., the fragmented (clipped) LMW species of the antibody is no more than 0.3% (w/w) of total protein or below the detection limit in the pharmaceutical composition. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 12 weeks storage in the presence of 0.1-5.0 ppm H2O2 at 25±5° C., the fragmented (clipped) LMW species of the antibody is no more than 0.3% (w/w) of total protein or below the detection limit in the pharmaceutical composition.

In one embodiment, the pharmaceutical compositions provided herein remain stable and have a density of subvisible particles that meets the US Pharmacopeia (USP) standard for intravenous administration after storage over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In one embodiment, the pharmaceutical compositions remain stable and have a density of subvisible particles that meets the US Pharmacopeia (USP) standard for intravenous administration after storage over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In one embodiment, the pharmaceutical compositions remain stable and have a density of subvisible particles that meets the US Pharmacopeia (USP) standard for intravenous administration after storage over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40±5° C. In one embodiment, the pharmaceutical compositions provided herein remain stable and have a density of subvisible particles that meets the US Pharmacopeia (USP) standard for intravenous administration after storage over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C.

In one embodiment, the pharmaceutical compositions provided herein have a density of subvisible particles that meets the USP standard for intravenous administration after storage over 1 week, 2 weeks, 4 weeks, 2 months, 3 months, 4 months, 5 months, 6 months, 26 weeks, 7 months, 8 months, 9 months, 10 months, 11 months, 12 months, 14 months, 24 months, or longer at 5±3° C. In one embodiment, the pharmaceutical compositions have a density of ≥2 μm subvisible particles of less than about 10,000 counts/ml, 5,000 counts/ml, 2,500 counts/ml, 2,000 counts/ml, 1,500 counts/ml, 1,000 counts/ml, 900 counts/ml, 800 counts/ml, 700 counts/ml, 600 counts/ml, 500 counts/ml, 400 counts/ml, 300 counts/ml, 200 counts/ml, or 100 counts/ml when stored at 5±3° C. In one embodiment, the pharmaceutical compositions have a density of ≥10 μm subvisible particles of less than about 1,000 counts/ml, 900 counts/ml, 800 counts/ml, 700 counts/ml, 600 counts/ml, 500 counts/ml, 400 counts/ml, 300 counts/ml, 200 counts/ml, 100 counts/ml, 90 counts/ml, 80 counts/ml, 70 counts/ml, 60 counts/ml, 50 counts/ml, 40 counts/ml, 30 counts/ml, 20 counts/ml, or 10 counts/ml when stored at 5±3° C. In one embodiment, the pharmaceutical compositions have a density of ≥25 μm subvisible particles of less than about 100 counts/ml, 50 counts/ml, 40 counts/ml, 30 counts/ml, 20 counts/ml, 10 counts/ml, or 5 counts/ml when stored at 5±3° C.

In one embodiment, the pharmaceutical compositions provided herein have a density of subvisible particles that meets the USP standard for intravenous administration after storage over 1 week, 2 weeks, 4 weeks, 2 months, 3 months, 4 months, 5 months, 6 months, 26 weeks, 7 months, 8 months, 9 months, 10 months, 11 months, 12 months, 14 months, 24 months, or longer at 25±5° C. In one embodiment, the pharmaceutical compositions have a density of ≥2 μm subvisible particles of less than about 10,000 counts/ml, 5,000 counts/ml, 2,500 counts/ml, 2,000 counts/ml, 1,500 counts/ml, 1,000 counts/ml, 900 counts/ml, 800 counts/ml, 700 counts/ml, 600 counts/ml, 500 counts/ml, 400 counts/ml, 300 counts/ml, 200 counts/ml, or 100 counts/ml when stored at 25±5° C. In one embodiment, the pharmaceutical compositions have a density of ≥10 μm subvisible particles of less than about 1,000 counts/ml, 900 counts/ml, 800 counts/ml, 700 counts/ml, 600 counts/ml, 500 counts/ml, 400 counts/ml, 300 counts/ml, 200 counts/ml, 100 counts/ml, 90 counts/ml, 80 counts/ml, 70 counts/ml, 60 counts/ml, 50 counts/ml, 40 counts/ml, 30 counts/ml, 20 counts/ml, or 10 counts/ml when stored at 25±5° C. In one embodiment, the pharmaceutical compositions have a density of ≥25 μm subvisible particles of less than about 100 counts/ml, 50 counts/ml, 40 counts/ml, 30 counts/ml, 20 counts/ml, 10 counts/ml, or 5 counts/ml when stored at 25±5° C.

In one embodiment, the pharmaceutical compositions provided herein have a density of subvisible particles that meets the USP standard for intravenous administration after storage over 1 week, 2 weeks, 4 weeks, 2 months, 3 months, 4 months, 5 months, 6 months, 26 weeks, 7 months, 8 months, 9 months, 10 months, 11 months, 12 months, 14 months, 24 months, or longer at 40±5° C. In one embodiment, the pharmaceutical compositions have a density of ≥2 μm subvisible particles of less than about 10,000 counts/ml, 5,000 counts/ml, 2,500 counts/ml, 2,000 counts/ml, 1,500 counts/ml, 1,000 counts/ml, 900 counts/ml, 800 counts/ml, 700 counts/ml, 600 counts/ml, 500 counts/ml, 400 counts/ml, 300 counts/ml, 200 counts/ml, or 100 counts/ml when stored at 40±5° C. In one embodiment, the pharmaceutical compositions have a density of ≥10 μm subvisible particles of less than about 1,000 counts/ml, 900 counts/ml, 800 counts/ml, 700 counts/ml, 600 counts/ml, 500 counts/ml, 400 counts/ml, 300 counts/ml, 200 counts/ml, 100 counts/ml, 90 counts/ml, 80 counts/ml, 70 counts/ml, 60 counts/ml, 50 counts/ml, 40 counts/ml, 30 counts/ml, 20 counts/ml, or 10 counts/ml when stored at 40±5° C. In one embodiment, the pharmaceutical compositions have a density of ≥25 μm subvisible particles of less than about 100 counts/ml, 50 counts/ml, 40 counts/ml, 30 counts/ml, 20 counts/ml, 10 counts/ml, or 5 counts/ml when stored at 40±5° C.

In one embodiment, the pharmaceutical compositions provided herein have a density of subvisible particles that meets the USP standard for intravenous administration after storage over 1 week, 2 weeks, 4 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In one embodiment, the pharmaceutical compositions have a density of ≥2 μm subvisible particles of less than about 10,000 counts/ml, 5,000 counts/ml, 2,500 counts/ml, 2,000 counts/ml, 1,500 counts/ml, 1,000 counts/ml, 900 counts/ml, 800 counts/ml, 700 counts/ml, 600 counts/ml, 500 counts/ml, 400 counts/ml, 300 counts/ml, 200 counts/ml, or 100 counts/ml when stored in the presence of 0.1-5.0 ppm H2O2 at 25±5° C. In one embodiment, the pharmaceutical compositions have a density of ≥10 μm subvisible particles of less than about 1,000 counts/ml, 900 counts/ml, 800 counts/ml, 700 counts/ml, 600 counts/ml, 500 counts/ml, 400 counts/ml, 300 counts/ml, 200 counts/ml, 100 counts/ml, 90 counts/ml, 80 counts/ml, 70 counts/ml, 60 counts/ml, 50 counts/ml, 40 counts/ml, 30 counts/ml, 20 counts/ml, or 10 counts/ml when stored in the presence of 0.1-5.0 ppm H2O2 at 25±5° C. In one embodiment, the pharmaceutical compositions have a density of ≥25 μm subvisible particles of less than about 100 counts/ml, 50 counts/ml, 40 counts/ml, 30 counts/ml, 20 counts/ml, 10 counts/ml, or 5 counts/ml when stored in the presence of 0.1-5.0 ppm H2O2 at 25±5° C.

In one embodiment, the pharmaceutical compositions provided herein after up to 3 days of agitation (e.g. agitation at 100 RPM) at room temperature (e.g. 25±5° C.) have a density of ≥2 μm subvisible particles of less than about 500 counts/ml. In one embodiment, the pharmaceutical compositions provided herein after up to 3 days of agitation (e.g. agitation at 100 RPM) at room temperature (e.g. 25±5° C.) have a density of ≥2 μm subvisible particles of less than about 600 counts/ml. In one embodiment, the pharmaceutical compositions provided herein after up to 3 days of agitation (e.g. agitation at 100 RPM) at room temperature (e.g. 25±5° C.) have a density of ≥2 μm subvisible particles of less than about 700 counts/ml. In one embodiment, the pharmaceutical compositions provided herein after up to 3 days of agitation (e.g. agitation at 100 RPM) at room temperature (e.g. 25±5° C.) have a density of ≥2 μm subvisible particles of less than about 800 counts/ml. In one embodiment, the pharmaceutical compositions provided herein after up to 3 days of agitation (e.g. agitation at 100 RPM) at room temperature (e.g. 25±5° C.) have a density of ≥2 μm subvisible particles of less than about 900 counts/ml. In one embodiment, the pharmaceutical compositions provided herein after up to 3 days of agitation (e.g. agitation at 100 RPM) at room temperature (e.g. 25±5° C.) have a density of ≥2 μm subvisible particles of less than about 1,000 counts/ml. In one embodiment, the pharmaceutical compositions after up to 1 day of agitation (e.g. agitation at 100 RPM) at room temperature (e.g. 25±5° C.) have a density of ≥2 μm subvisible particles of less than about 300 counts/ml. In one embodiment, the pharmaceutical compositions after up to 1 day of agitation (e.g. agitation at 100 RPM) at room temperature (e.g. 25±5° C.) have a density of ≥2 μm subvisible particles of less than about 200 counts/ml. In one embodiment, the pharmaceutical compositions after up to 1 day of agitation (e.g. agitation at 100 RPM) at room temperature (e.g. 25±5° C.) have a density of ≥2 μm subvisible particles of less than about 100 counts/ml. In one embodiment, the pharmaceutical compositions after up to 1 day of agitation (e.g. agitation at 100 RPM) at room temperature (e.g. 25±5° C.) have a density of ≥2 μm subvisible particles of less than about 90 counts/ml. In one embodiment, the pharmaceutical compositions after up to 1 day of agitation (e.g. agitation at 100 RPM) at room temperature (e.g. 25±5° C.) have a density of ≥2 μm subvisible particles of less than about 80 counts/ml. In one embodiment, the pharmaceutical compositions after up to 1 day of agitation (e.g. agitation at 100 RPM) at room temperature (e.g. 25±5° C.) have a density of ≥2 μm subvisible particles of less than about 70 counts/ml. In one embodiment, the pharmaceutical compositions after up to 1 day of agitation (e.g. agitation at 100 RPM) at room temperature (e.g. 25±5° C.) have a density of ≥2 μm subvisible particles of less than about 60 counts/ml. In one embodiment, the pharmaceutical compositions after up to 1 day of agitation (e.g. agitation at 100 RPM) at room temperature (e.g. 25±5° C.) have a density of ≥2 μm subvisible particles of less than about 50 counts/ml. In one embodiment, the pharmaceutical compositions after up to 1 day of agitation (e.g. agitation at 100 RPM) at room temperature (e.g. 25±5° C.) have a density of ≥2 μm subvisible particles of less than about 40 counts/ml. In one embodiment, the pharmaceutical compositions after up to 1 day of agitation (e.g. agitation at 100 RPM) at room temperature (e.g. 25±5° C.) have a density of ≥2 μm subvisible particles of less than about 30 counts/ml. In one embodiment, the pharmaceutical compositions after up to 1 day of agitation (e.g. agitation at 100 RPM) at room temperature (e.g. 25±5° C.) have a density of ≥2 μm subvisible particles is below the detection limit in an HIAC system.

In one embodiment, the pharmaceutical compositions provided herein after up to 10 cycles of freeze and thaw from −80° C. to room temperature have a density of ≥2 μm subvisible particles of less than about 1,000 counts/ml. In one embodiment, the pharmaceutical compositions after up to 10 cycles of freeze and thaw from −80° C. to room temperature have a density of ≥2 μm subvisible particles of less than about 900 counts/ml. In one embodiment, the pharmaceutical compositions after up to 10 cycles of freeze and thaw from −80° C. to room temperature have a density of ≥2 μm subvisible particles of less than about 800 counts/ml. In one embodiment, the pharmaceutical compositions after up to 10 cycles of freeze and thaw from −80° C. to room temperature have a density of ≥2 μm subvisible particles of less than about 700 counts/ml. In one embodiment, the pharmaceutical compositions after up to 10 cycles of freeze and thaw from −80° C. to room temperature have a density of ≥2 μm subvisible particles of less than about 600 counts/ml. In one embodiment, the pharmaceutical compositions after up to 10 cycles of freeze and thaw from −80° C. to room temperature have a density of ≥2 μm subvisible particles of less than about 500 counts/ml. In one embodiment, the pharmaceutical compositions provided herein after up to 10 cycles of freeze and thaw from −80° C. to room temperature have a density of ≥2 μm subvisible particles of less than about 400 counts/ml. In one embodiment, the pharmaceutical compositions after up to 10 cycles of freeze and thaw from −80° C. to room temperature have a density of ≥2 μm subvisible particles of less than about 300 counts/ml. In one embodiment, the pharmaceutical compositions after up to 10 cycles of freeze and thaw from −80° C. to room temperature have a density of ≥2 μm subvisible particles of less than about 200 counts/ml. In one embodiment, the pharmaceutical compositions after up to 10 cycles of freeze and thaw from −80° C. to room temperature have a density of ≥2 μm subvisible particles of less than about 100 counts/ml. In one embodiment, the pharmaceutical compositions after up to 10 cycles of freeze and thaw from −80° C. to room temperature have a density of ≥2 μm subvisible particles of less than about 90 counts/ml. In one embodiment, the pharmaceutical compositions after up to 10 cycles of freeze and thaw from −80° C. to room temperature have a density of ≥2 μm subvisible particles of less than about 80 counts/ml. In one embodiment, the pharmaceutical compositions after up to 10 cycles of freeze and thaw from −80° C. to room temperature have a density of ≥2 μm subvisible particles of less than about 70 counts/ml. In one embodiment, the pharmaceutical compositions after up to 10 cycles of freeze and thaw from −80° C. to room temperature have a density of ≥2 μm subvisible particles of less than about 60 counts/ml. In one embodiment, the pharmaceutical compositions after up to 10 cycles of freeze and thaw from −80° C. to room temperature have a density of ≥2 μm subvisible particles of less than about 50 counts/ml. In one embodiment, the pharmaceutical compositions after up to 10 cycles of freeze and thaw from −80° C. to room temperature have a density of ≥2 μm subvisible particles of less than about 40 counts/ml. In one embodiment, the pharmaceutical compositions after up to 10 cycles of freeze and thaw from −80° C. to room temperature have a density of ≥2 μm subvisible particles of less than about 30 counts/ml.

In one embodiment, the pharmaceutical compositions provided herein after up to 3 days of agitation (e.g. agitation at 100 RPM) at room temperature (e.g. 25±5° C.) have a density of ≥10 μm subvisible particles of less than about 1,000 counts/ml. In one embodiment, the pharmaceutical compositions after up to 3 days of agitation (e.g. agitation at 100 RPM) at room temperature (e.g. 25±5° C.) have a density of ≥10 μm subvisible particles of less than about 900 counts/ml. In one embodiment, the pharmaceutical compositions after up to 3 days of agitation (e.g. agitation at 100 RPM) at room temperature (e.g. 25±5° C.) have a density of ≥10 μm subvisible particles of less than about 800 counts/ml. In one embodiment, the pharmaceutical compositions after up to 3 days of agitation (e.g. agitation at 100 RPM) at room temperature (e.g. 25±5° C.) have a density of ≥10 μm subvisible particles of less than about 700 counts/ml. In one embodiment, the pharmaceutical compositions after up to 3 days of agitation (e.g. agitation at 100 RPM) at room temperature (e.g. 25±5° C.) have a density of ≥10 μm subvisible particles of less than about 600 counts/ml. In one embodiment, the pharmaceutical compositions after up to 3 days of agitation (e.g. agitation at 100 RPM) at room temperature (e.g. 25±5° C.) have a density of ≥10 μm subvisible particles of less than about 500 counts/ml. In one embodiment, the pharmaceutical compositions after up to 3 days of agitation (e.g. agitation at 100 RPM) at room temperature (e.g. 25±5° C.) have a density of ≥10 μm subvisible particles of less than about 400 counts/ml. In one embodiment, the pharmaceutical compositions after up to 3 days of agitation (e.g. agitation at 100 RPM) at room temperature (e.g. 25±5° C.) have a density of ≥10 μm subvisible particles of less than about 300 counts/ml. In one embodiment, the pharmaceutical compositions after up to 3 days of agitation (e.g. agitation at 100 RPM) at room temperature (e.g. 25±5° C.) have a density of ≥10 μm subvisible particles of less than about 200 counts/ml. In one embodiment, the pharmaceutical compositions after up to 3 days of agitation (e.g. agitation at 100 RPM) at room temperature (e.g. 25±5° C.) have a density of ≥10 μm subvisible particles of less than about 100 counts/ml. In one embodiment, the pharmaceutical compositions after up to 3 days of agitation (e.g. agitation at 100 RPM) at room temperature (e.g. 25±5° C.) have a density of ≥10 μm subvisible particles of less than about 90 counts/ml. In one embodiment, the pharmaceutical compositions after up to 3 days of agitation (e.g. agitation at 100 RPM) at room temperature (e.g. 25±5° C.) have a density of ≥10 μm subvisible particles of less than about 80 counts/ml. In one embodiment, the pharmaceutical compositions after up to 3 days of agitation (e.g. agitation at 100 RPM) at room temperature (e.g. 25±5° C.) have a density of ≥10 μm subvisible particles of less than about 70 counts/ml. In one embodiment, the pharmaceutical compositions after up to 3 days of agitation (e.g. agitation at 100 RPM) at room temperature (e.g. 25±5° C.) have a density of ≥10 μm subvisible particles of less than about 60 counts/ml. In one embodiment, the pharmaceutical compositions after up to 3 days of agitation (e.g. agitation at 100 RPM) at room temperature (e.g. 25±5° C.) have a density of ≥10 μm subvisible particles of less than about 50 counts/ml. In one embodiment, the pharmaceutical compositions after up to 3 days of agitation (e.g. agitation at 100 RPM) at room temperature (e.g. 25±5° C.) have a density of ≥10 μm subvisible particles of less than about 40 counts/ml. In one embodiment, the pharmaceutical compositions after up to 3 days of agitation (e.g. agitation at 100 RPM) at room temperature (e.g. 25±5° C.) have a density of ≥10 μm subvisible particles of less than about 30 counts/ml. In one embodiment, the pharmaceutical compositions after up to 3 days of agitation (e.g. agitation at 100 RPM) at room temperature (e.g. 25±5° C.) have a density of ≥10 μm subvisible particles of less than about 20 counts/ml. In one embodiment, the pharmaceutical compositions after up to 3 days of agitation (e.g. agitation at 100 RPM) at room temperature (e.g. 25±5° C.) have a density of ≥10 μm subvisible particles of less than about 10 counts/ml. In one embodiment, the pharmaceutical compositions after up to 3 days of agitation (e.g. agitation at 100 RPM) at room temperature (e.g. 25±5° C.) have a density of ≥10 μm subvisible particles of less than about 5 counts/ml.

In one embodiment, the pharmaceutical compositions after equal to or up to 1 day of agitation (e.g. agitation at 100 RPM) at room temperature (e.g. 25±5° C.) have a density of ≥10 μm subvisible particles of less than about 100 counts/ml. In one embodiment, the pharmaceutical compositions after equal to or up to 1 day of agitation (e.g. agitation at 100 RPM) at room temperature (e.g. 25±5° C.) have a density of ≥10 μm subvisible particles of less than about 90 counts/ml. In one embodiment, the pharmaceutical compositions after equal to or up to 1 day of agitation (e.g. agitation at 100 RPM) at room temperature (e.g. 25±5° C.) have a density of ≥10 μm subvisible particles of less than about 80 counts/ml. In one embodiment, the pharmaceutical compositions after equal to or up to 1 day of agitation (e.g. agitation at 100 RPM) at room temperature (e.g. 25±5° C.) have a density of ≥10 μm subvisible particles of less than about 70 counts/ml. In one embodiment, the pharmaceutical compositions after equal to or up to 1 day of agitation (e.g. agitation at 100 RPM) at room temperature (e.g. 25±5° C.) have a density of ≥10 μm subvisible particles of less than about 60 counts/ml. In one embodiment, the pharmaceutical compositions after equal to or up to 1 day of agitation (e.g. agitation at 100 RPM) at room temperature (e.g. 25±5° C.) have a density of ≥10 μm subvisible particles of less than about 50 counts/ml. In one embodiment, the pharmaceutical compositions after equal to or up to 1 day of agitation (e.g. agitation at 100 RPM) at room temperature (e.g. 25±5° C.) have a density of ≥10 μm subvisible particles of less than about 40 counts/ml. In one embodiment, the pharmaceutical compositions after equal to or up to 1 day of agitation (e.g. agitation at 100 RPM) at room temperature (e.g. 25±5° C.) have a density of ≥10 μm subvisible particles of less than about 30 counts/ml. In one embodiment, the pharmaceutical compositions after equal to or up to 1 day of agitation (e.g. agitation at 100 RPM) at room temperature (e.g. 25±5° C.) have a density of ≥10 μm subvisible particles of less than about 20 counts/ml. In one embodiment, the pharmaceutical compositions after equal to or up to 1 day of agitation (e.g. agitation at 100 RPM) at room temperature (e.g. 25±5° C.) have a density of ≥10 μm subvisible particles of less than about 10 counts/ml. In one embodiment, the pharmaceutical compositions after equal to or up to 1 day of agitation (e.g. agitation at 100 RPM) at room temperature (e.g. 25±5° C.) have a density of ≥10 μm subvisible particles of less than about 5 counts/ml. In one embodiment, the pharmaceutical compositions after equal to or up to 1 day of agitation (e.g. agitation at 100 RPM) at room temperature (e.g. 25±5° C.) have a density of ≥10 μm subvisible particles below the detection limit in an HIAC system.

In one embodiment, the pharmaceutical compositions provided herein after up to 10 cycles of freeze and thaw from −80° C. to room temperature have a density of ≥10 μm subvisible particles of less than about 1,000 counts/ml. In one embodiment, the pharmaceutical compositions after up to 10 cycles of freeze and thaw from −80° C. to room temperature have a density of ≥10 μm subvisible particles of less than about 900 counts/ml. In one embodiment, the pharmaceutical compositions after up to 10 cycles of freeze and thaw from −80° C. to room temperature have a density of ≥10 μm subvisible particles of less than about 800 counts/ml. In one embodiment, the pharmaceutical compositions after up to 10 cycles of freeze and thaw from −80° C. to room temperature have a density of ≥10 μm subvisible particles of less than about 700 counts/ml. In one embodiment, the pharmaceutical compositions after up to 10 cycles of freeze and thaw from −80° C. to room temperature have a density of ≥10 μm subvisible particles of less than about 600 counts/ml. In one embodiment, the pharmaceutical compositions after up to 10 cycles of freeze and thaw from −80° C. to room temperature have a density of ≥10 μm subvisible particles of less than about 500 counts/ml. In one embodiment, the pharmaceutical compositions after up to 10 cycles of freeze and thaw from −80° C. to room temperature have a density of ≥10 μm subvisible particles of less than about 400 counts/ml. In one embodiment, the pharmaceutical compositions after up to 10 cycles of freeze and thaw from −80° C. to room temperature have a density of ≥10 μm subvisible particles of less than about 300 counts/ml. In one embodiment, the pharmaceutical compositions after up to 10 cycles of freeze and thaw from −80° C. to room temperature have a density of ≥10 μm subvisible particles of less than about 200 counts/ml. In one embodiment, the pharmaceutical compositions after up to 10 cycles of freeze and thaw from −80° C. to room temperature have a density of ≥10 μm subvisible particles of less than about 100 counts/ml. In one embodiment, the pharmaceutical compositions after up to 10 cycles of freeze and thaw from −80° C. to room temperature have a density of ≥10 μm subvisible particles of less than about 90 counts/ml. In one embodiment, the pharmaceutical compositions after up to 10 cycles of freeze and thaw from −80° C. to room temperature have a density of ≥10 μm subvisible particles of less than about 80 counts/ml. In one embodiment, the pharmaceutical compositions after up to 10 cycles of freeze and thaw from −80° C. to room temperature have a density of ≥10 μm subvisible particles of less than about 70 counts/ml. In one embodiment, the pharmaceutical compositions after up to 10 cycles of freeze and thaw from −80° C. to room temperature have a density of ≥10 μm subvisible particles of less than about 60 counts/ml. In one embodiment, the pharmaceutical compositions after up to 10 cycles of freeze and thaw from −80° C. to room temperature have a density of ≥10 μm subvisible particles of less than about 50 counts/ml. In one embodiment, the pharmaceutical compositions after up to 10 cycles of freeze and thaw from −80° C. to room temperature have a density of ≥10 μm subvisible particles of less than about 40 counts/ml. In one embodiment, the pharmaceutical compositions after up to 10 cycles of freeze and thaw from −80° C. to room temperature have a density of ≥10 μm subvisible particles of less than about 30 counts/ml. In one embodiment, the pharmaceutical compositions after up to 10 cycles of freeze and thaw from −80° C. to room temperature have a density of ≥10 μm subvisible particles of less than about 20 counts/ml. In one embodiment, the pharmaceutical compositions after up to 10 cycles of freeze and thaw from −80° C. to room temperature have a density of ≥10 μm subvisible particles of less than about 10 counts/ml. In one embodiment, the pharmaceutical compositions after up to 10 cycles of freeze and thaw from −80° C. to room temperature have a density of ≥10 μm subvisible particles of less than about 5 counts/ml.

In one embodiment, the pharmaceutical compositions after up to 3 days of agitation (e.g. agitation at 100 RPM) at room temperature (e.g. 25±5° C.) have a density of ≥25 μm subvisible particles of less than about 100 counts/ml. In one embodiment, the pharmaceutical compositions after up to 3 days of agitation (e.g. agitation at 100 RPM) at room temperature (e.g. 25±5° C.) have a density of ≥25 μm subvisible particles of less than about 90 counts/ml. In one embodiment, the pharmaceutical compositions after up to 3 days of agitation (e.g. agitation at 100 RPM) at room temperature (e.g. 25±5° C.) have a density of ≥25 μm subvisible particles of less than about 80 counts/ml. In one embodiment, the pharmaceutical compositions after up to 3 days of agitation (e.g. agitation at 100 RPM) at room temperature (e.g. 25±5° C.) have a density of ≥25 μm subvisible particles of less than about 70 counts/ml. In one embodiment, the pharmaceutical compositions after up to 3 days of agitation (e.g. agitation at 100 RPM) at room temperature (e.g. 25±5° C.) have a density of ≥25 μm subvisible particles of less than about 60 counts/ml. In one embodiment, the pharmaceutical compositions after up to 3 days of agitation (e.g. agitation at 100 RPM) at room temperature (e.g. 25±5° C.) have a density of ≥25 μm subvisible particles of less than about 50 counts/ml. In one embodiment, the pharmaceutical compositions after up to 3 days of agitation (e.g. agitation at 100 RPM) at room temperature (e.g. 25±5° C.) have a density of ≥25 μm subvisible particles of less than about 40 counts/ml. In one embodiment, the pharmaceutical compositions after up to 3 days of agitation (e.g. agitation at 100 RPM) at room temperature (e.g. 25±5° C.) have a density of ≥25 μm subvisible particles of less than about 30 counts/ml. In one embodiment, the pharmaceutical compositions after up to 3 days of agitation (e.g. agitation at 100 RPM) at room temperature (e.g. 25±5° C.) have a density of ≥25 μm subvisible particles of less than about 20 counts/ml. In one embodiment, the pharmaceutical compositions after up to 3 days of agitation (e.g. agitation at 100 RPM) at room temperature (e.g. 25±5° C.) have a density of ≥25 μm subvisible particles of less than about 10 counts/ml. In one embodiment, the pharmaceutical compositions after up to 3 days of agitation (e.g. agitation at 100 RPM) at room temperature (e.g. 25±5° C.) have a density of ≥25 μm subvisible particles of less than about 5 counts/ml. In one embodiment, the pharmaceutical compositions after up to 3 days of agitation (e.g. agitation at 100 RPM) at room temperature (e.g. 25±5° C.) have a density of ≥25 μm subvisible particles of less than about 4 counts/ml. In one embodiment, the pharmaceutical compositions after up to 3 days of agitation (e.g. agitation at 100 RPM) at room temperature (e.g. 25±5° C.) have a density of ≥25 μm subvisible particles of less than about 3 counts/ml. In one embodiment, the pharmaceutical compositions after up to 3 days of agitation (e.g. agitation at 100 RPM) at room temperature (e.g. 25±5° C.) have a density of ≥25 μm subvisible particles of less than about 2 counts/ml. In one embodiment, the pharmaceutical compositions after up to 3 days of agitation (e.g. agitation at 100 RPM) at room temperature (e.g. 25±5° C.) have a density of ≥25 μm subvisible particles of less than about 1 counts/ml. In one embodiment, the pharmaceutical compositions after up to 3 days of agitation (e.g. agitation at 100 RPM) at room temperature (e.g. 25±5° C.) have a density of ≥25 μm subvisible particles below the detection limit of an HIAC system.

In one embodiment, the pharmaceutical compositions after equal to or up to 1 day of agitation (e.g. agitation at 100 RPM) at room temperature (e.g. 25±5° C.) have a density of ≥25 μm subvisible particles of less than about 100 counts/ml. In one embodiment, the pharmaceutical compositions after equal to or up to 1 day of agitation (e.g. agitation at 100 RPM) at room temperature (e.g. 25±5° C.) have a density of ≥25 μm subvisible particles of less than about 90 counts/ml. In one embodiment, the pharmaceutical compositions after equal to or up to 1 day of agitation (e.g. agitation at 100 RPM) at room temperature (e.g. 25±5° C.) have a density of ≥25 μm subvisible particles of less than about 80 counts/ml. In one embodiment, the pharmaceutical compositions after equal to or up to 1 day of agitation (e.g. agitation at 100 RPM) at room temperature (e.g. 25±5° C.) have a density of ≥25 μm subvisible particles of less than about 70 counts/ml. In one embodiment, the pharmaceutical compositions after equal to or up to 1 day of agitation (e.g. agitation at 100 RPM) at room temperature (e.g. 25±5° C.) have a density of ≥25 μm subvisible particles of less than about 60 counts/ml. In one embodiment, the pharmaceutical compositions after equal to or up to 1 day of agitation (e.g. agitation at 100 RPM) at room temperature (e.g. 25±5° C.) have a density of ≥25 μm subvisible particles of less than about 50 counts/ml. In one embodiment, the pharmaceutical compositions after equal to or up to 1 day of agitation (e.g. agitation at 100 RPM) at room temperature (e.g. 25±5° C.) have a density of ≥25 μm subvisible particles of less than about 40 counts/ml. In one embodiment, the pharmaceutical compositions after equal to or up to 1 day of agitation (e.g. agitation at 100 RPM) at room temperature (e.g. 25±5° C.) have a density of ≥25 μm subvisible particles of less than about 30 counts/ml. In one embodiment, the pharmaceutical compositions after equal to or up to 1 day of agitation (e.g. agitation at 100 RPM) at room temperature (e.g. 25±5° C.) have a density of ≥25 μm subvisible particles of less than about 20 counts/ml. In one embodiment, the pharmaceutical compositions after equal to or up to 1 day of agitation (e.g. agitation at 100 RPM) at room temperature (e.g. 25±5° C.) have a density of ≥25 μm subvisible particles of less than about 10 counts/ml. In one embodiment, the pharmaceutical compositions after equal to or up to 1 day of agitation (e.g. agitation at 100 RPM) at room temperature (e.g. 25±5° C.) have a density of ≥25 μm subvisible particles of less than about 5 counts/ml. In one embodiment, the pharmaceutical compositions after equal to or up to 1 day of agitation (e.g. agitation at 100 RPM) at room temperature (e.g. 25±5° C.) have a density of ≥25 μm subvisible particles of less than about 4 counts/ml. In one embodiment, the pharmaceutical compositions after equal to or up to 1 day of agitation (e.g. agitation at 100 RPM) at room temperature (e.g. 25±5° C.) have a density of ≥25 μm subvisible particles of less than about 3 counts/ml. In one embodiment, the pharmaceutical compositions after equal to or up to 1 day of agitation (e.g. agitation at 100 RPM) at room temperature (e.g. 25±5° C.) have a density of ≥25 μm subvisible particles of less than about 2 counts/ml. In one embodiment, the pharmaceutical compositions after equal to or up to 1 day of agitation (e.g. agitation at 100 RPM) at room temperature (e.g. 25±5° C.) have a density of ≥25 μm subvisible particles of less than about 1 count/ml. In one embodiment, the pharmaceutical compositions after equal to or up to 1 day of agitation (e.g. agitation at 100 RPM) at room temperature (e.g. 25±5° C.) have a density of ≥25 μm subvisible particles below the detection limit of a HIAC system.

In one embodiment, the pharmaceutical compositions provided herein after up to 10 cycles of freeze and thaw from −80° C. to room temperature have a density of ≥25 μm subvisible particles of less than about 1,000 counts/ml. In one embodiment, the pharmaceutical compositions after up to 10 cycles of freeze and thaw from −80° C. to room temperature have a density of ≥25 μm subvisible particles of less than about 900 counts/ml. In one embodiment, the pharmaceutical compositions after up to 10 cycles of freeze and thaw from −80° C. to room temperature have a density of ≥25 μm subvisible particles of less than about 800 counts/ml. In one embodiment, the pharmaceutical compositions after up to 10 cycles of freeze and thaw from −80° C. to room temperature have a density of ≥25 μm subvisible particles of less than about 700 counts/ml. In one embodiment, the pharmaceutical compositions after up to 10 cycles of freeze and thaw from −80° C. to room temperature have a density of ≥25 μm subvisible particles of less than about 600 counts/ml. In one embodiment, the pharmaceutical compositions after up to 10 cycles of freeze and thaw from −80° C. to room temperature have a density of ≥25 μm subvisible particles of less than about 500 counts/ml. In one embodiment, the pharmaceutical compositions after up to 10 cycles of freeze and thaw from −80° C. to room temperature have a density of ≥25 μm subvisible particles of less than about 400 counts/ml. In one embodiment, the pharmaceutical compositions after up to 10 cycles of freeze and thaw from −80° C. to room temperature have a density of ≥25 μm subvisible particles of less than about 300 counts/ml. In one embodiment, the pharmaceutical compositions after up to 10 cycles of freeze and thaw from −80° C. to room temperature have a density of ≥25 μm subvisible particles of less than about 200 counts/ml. In one embodiment, the pharmaceutical compositions after up to 10 cycles of freeze and thaw from −80° C. to room temperature have a density of ≥25 μm subvisible particles of less than about 100 counts/ml. In one embodiment, the pharmaceutical compositions after up to 10 cycles of freeze and thaw from −80° C. to room temperature have a density of ≥25 μm subvisible particles of less than about 90 counts/ml. In one embodiment, the pharmaceutical compositions after up to 10 cycles of freeze and thaw from −80° C. to room temperature have a density of ≥25 μm subvisible particles of less than about 80 counts/ml. In one embodiment, the pharmaceutical compositions after up to 10 cycles of freeze and thaw from −80° C. to room temperature have a density of ≥25 μm subvisible particles of less than about 70 counts/ml. In one embodiment, the pharmaceutical compositions after up to 10 cycles of freeze and thaw from −80° C. to room temperature have a density of ≥25 μm subvisible particles of less than about 60 counts/ml. In one embodiment, the pharmaceutical compositions after up to 10 cycles of freeze and thaw from −80° C. to room temperature have a density of ≥25 μm subvisible particles of less than about 50 counts/ml. In one embodiment, the pharmaceutical compositions after up to 10 cycles of freeze and thaw from −80° C. to room temperature have a density of ≥25 μm subvisible particles of less than about 40 counts/ml. In one embodiment, the pharmaceutical compositions after up to 10 cycles of freeze and thaw from −80° C. to room temperature have a density of ≥25 μm subvisible particles of less than about 30 counts/ml. In one embodiment, the pharmaceutical compositions after up to 10 cycles of freeze and thaw from −80° C. to room temperature have a density of ≥25 μm subvisible particles of less than about 20 counts/ml. In one embodiment, the pharmaceutical compositions after up to 10 cycles of freeze and thaw from −80° C. to room temperature have a density of ≥25 μm subvisible particles of less than about 10 counts/ml. In one embodiment, the pharmaceutical compositions after up to 10 cycles of freeze and thaw from −80° C. to room temperature have a density of ≥25 μm subvisible particles of less than about 5 counts/ml.

In a specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 1 or 3 days of agitation (e.g. agitation at 100 RPM) at room temperature, the pharmaceutical composition has a density of ≥2 μm subvisible particles less than about 500 counts/ml, and a density of ≥10 μm subvisible particles less than about 100 counts/ml, and a density of ≥25 μm subvisible particles less than about 5 counts/ml. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 3 days of vigorous agitation (e.g. agitation at 300 RPM) at room temperature, the pharmaceutical composition has a density of ≥2 μm subvisible particles less than about 500 counts/ml, and a density of ≥10 μm subvisible particles less than about 100 counts/ml, and a density of ≥25 μm subvisible particles less than about 5 counts/ml. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, and (iii) 0.02% (w/v) polysorbate-20, and wherein after 3, 5, or 10 cycles of freeze and thaw from −80° C. to room temperature, the pharmaceutical composition has a density of ≥2 μm subvisible particles less than about 500 counts/ml, and a density of ≥10 μm subvisible particles less than about 100 counts/ml, and a density of ≥25 μm subvisible particles less than about 5 counts/ml.

In a specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 1 or 3 days of agitation (e.g. agitation at 100 RPM) at room temperature, the pharmaceutical composition has a density of ≥2 μm subvisible particles less than about 500 counts/ml, and a density of ≥10 μm subvisible particles less than about 100 counts/ml, and a density of ≥25 μm subvisible particles less than about 5 counts/ml. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 3 days of vigorous agitation (e.g. agitation at 300 RPM) at room temperature, the pharmaceutical composition has a density of ≥2 μm subvisible particles less than about 500 counts/ml, and a density of ≥10 μm subvisible particles less than about 100 counts/ml, and a density of ≥25 μm subvisible particles less than about 5 counts/ml. In another specific embodiment, provided herein is a pharmaceutical composition comprising an antibody or antigen binding fragment that binds to CD30L, wherein the pharmaceutical composition has a pH of 5.5 and comprises (i) 20 mM histidine buffer, (ii) 100 mM arginine monohydrochloride, (iii) 10 mM L-Methionine, and (iv) 0.02% (w/v) polysorbate-20, and wherein after 3, 5, or 10 cycles of freeze and thaw from −80° C. to room temperature, the pharmaceutical composition has a density of ≥2 μm subvisible particles less than about 500 counts/ml, and a density of ≥10 μm subvisible particles less than about 100 counts/ml, and a density of ≥25 μm subvisible particles less than about 5 counts/ml.

In some embodiments, the pharmaceutical compositions provided herein have a stable pH value, wherein the pH changes no more than 0.1 unit over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In some embodiments, the pharmaceutical compositions have a stable pH value, wherein the pH changes no more than 0.2 unit over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In some embodiments, the pharmaceutical compositions have a stable pH value, wherein the pH changes no more than 0.1 unit over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In some embodiments, the pharmaceutical compositions have a stable pH value, wherein the pH changes no more than 0.2 unit over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In some embodiments, the pharmaceutical compositions have a stable pH value, wherein the pH changes no more than 0.1 unit over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40±5° C. In some embodiments, the pharmaceutical compositions have a stable pH value, wherein the pH changes no more than 0.2 unit over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40±5° C. In some embodiments, the pharmaceutical compositions provided herein have a stable pH value, wherein the pH changes no more than 0.1 unit over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In some embodiments, the pharmaceutical compositions have a stable pH value, wherein the pH changes no more than 0.2 unit over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In some embodiments of this paragraph, the starting pH of the pharmaceutical compositions is 5.0. In some embodiments of this paragraph, the starting pH of the pharmaceutical compositions is 5.1. In some embodiments of this paragraph, the starting pH of the pharmaceutical compositions is 5.2. In some embodiments of this paragraph, the starting pH of the pharmaceutical compositions is 5.3. In some embodiments of this paragraph, the starting pH of the pharmaceutical compositions is 5.4. In some embodiments of this paragraph, the starting pH of the pharmaceutical compositions is 5.5. In some embodiments of this paragraph, the starting pH of the pharmaceutical compositions is 5.5. In some embodiments of this paragraph, the starting pH of the pharmaceutical compositions is 5.7. In some embodiments of this paragraph, the starting pH of the pharmaceutical compositions is 5.8. In some embodiments of this paragraph, the starting pH of the pharmaceutical compositions is 5.9. In some embodiments of this paragraph, the starting pH of the pharmaceutical compositions is 6.0.

In some embodiments, the pharmaceutical compositions provided herein have a stable pI value, wherein the pI changes no more than 0.1 unit over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In some embodiments, the pharmaceutical compositions have a stable pI value, wherein the pI changes no more than 0.2 unit over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In some embodiments, the pharmaceutical compositions have a stable pI value, wherein the pI changes no more than 0.1 unit over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In some embodiments, the pharmaceutical compositions have a stable pI value, wherein the pI changes no more than 0.2 unit over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In some embodiments, the pharmaceutical compositions have a stable pI value, wherein the pI changes no more than 0.1 unit over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40±5° C. In some embodiments, the pharmaceutical compositions have a stable pI value, wherein the pI changes no more than 0.2 unit over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40±5° C. In some embodiments, the pharmaceutical compositions provided herein have a stable pI value, wherein the pI changes no more than 0.1 unit over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In some embodiments, the pharmaceutical compositions have a stable pI value, wherein the pI changes no more than 0.2 unit over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In some embodiments of this paragraph, the starting pI of the antibody or antigen binding fragments of the pharmaceutical compositions provided herein is 8.36.

In some embodiments, the pharmaceutical compositions provided herein have a stable concentration of the antibody or antigen binding fragment, wherein the concentration of the antibody or antigen binding fragment changes no more than 1 mg/ml over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In some embodiments, the pharmaceutical compositions have a stable concentration of the antibody or antigen binding fragment, wherein the concentration of the antibody or antigen binding fragment changes no more than 2 mg/ml over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In some embodiments, the pharmaceutical compositions have a stable concentration of the antibody or antigen binding fragment, wherein the concentration of the antibody or antigen binding fragment changes no more than 5% over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In some embodiments, the pharmaceutical compositions have a stable concentration of the antibody or antigen binding fragment, wherein the concentration of the antibody or antigen binding fragment changes no more than 1% over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In some embodiments, the pharmaceutical compositions have a stable concentration of the antibody or antigen binding fragment, wherein the concentration of the antibody or antigen binding fragment changes no more than 1 mg/ml over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In some embodiments, the pharmaceutical compositions have a stable concentration of the antibody or antigen binding fragment, wherein the concentration of the antibody or antigen binding fragment changes no more than 2 mg/ml over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In some embodiments, the pharmaceutical compositions have a stable concentration of the antibody or antigen binding fragment, wherein the concentration of the antibody or antigen binding fragment changes no more than 5% over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In some embodiments, the pharmaceutical compositions have a stable concentration of the antibody or antigen binding fragment, wherein the concentration of the antibody or antigen binding fragment changes no more than 1% over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In some embodiments, the pharmaceutical compositions have a stable concentration of the antibody or antigen binding fragment, wherein the concentration of the antibody or antigen binding fragment changes no more than 1 mg/ml over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40±5° C. In some embodiments, the pharmaceutical compositions have a stable concentration of the antibody or antigen binding fragment, wherein the concentration of the antibody or antigen binding fragment changes no more than 2 mg/ml over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40±5° C. In some embodiments, the pharmaceutical compositions have a stable concentration of the antibody or antigen binding fragment, wherein the concentration of the antibody or antigen binding fragment changes no more than 5% over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40±5° C. In some embodiments, the pharmaceutical compositions have a stable concentration of the antibody or antigen binding fragment, wherein the concentration of the antibody or antigen binding fragment changes no more than 1% over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40±5° C. In some embodiments, the pharmaceutical compositions provided herein have a stable concentration of the antibody or antigen binding fragment, wherein the concentration of the antibody or antigen binding fragment changes no more than 1 mg/ml over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In some embodiments, the pharmaceutical compositions have a stable concentration of the antibody or antigen binding fragment, wherein the concentration of the antibody or antigen binding fragment changes no more than 2 mg/ml over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In some embodiments, the pharmaceutical compositions have a stable concentration of the antibody or antigen binding fragment, wherein the concentration of the antibody or antigen binding fragment changes no more than 5% over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In some embodiments, the pharmaceutical compositions have a stable concentration of the antibody or antigen binding fragment, wherein the concentration of the antibody or antigen binding fragment changes no more than 1% over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In some embodiments of this paragraph, the starting concentration of the antibody or antigen binding fragments of the pharmaceutical compositions provided herein is about 200 mg/ml. In some embodiments of this paragraph, the starting concentration of the antibody or antigen binding fragments of the pharmaceutical compositions provided herein is at least 200 mg/ml.

In some embodiments, the purity of the antibody or antigen binding fragment in the pharmaceutical compositions provided herein remains stable during storage, wherein the purity changes no more than 1 percentage point over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In some embodiments, the purity of the antibody or antigen binding fragment in the pharmaceutical compositions remains stable during storage, wherein the purity changes no more than 2 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In some embodiments, the purity of the antibody or antigen binding fragment in the pharmaceutical compositions remains stable during storage, wherein the purity changes no more than 3 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In some embodiments, the purity of the antibody or antigen binding fragment in the pharmaceutical compositions remains stable during storage, wherein the purity changes no more than 4 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In some embodiments, the purity of the antibody or antigen binding fragment in the pharmaceutical compositions remains stable during storage, wherein the purity changes no more than 5 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In some embodiments of this paragraph, the starting purity of the antibody or antigen binding fragments of the pharmaceutical compositions is 95%. In some embodiments of this paragraph, the starting purity of the antibody or antigen binding fragments of the pharmaceutical compositions is 96%. In some embodiments of this paragraph, the starting purity of the antibody or antigen binding fragments of the pharmaceutical compositions is 97%. In some embodiments of this paragraph, the starting purity of the antibody or antigen binding fragments of the pharmaceutical compositions is 98%. In some embodiments of this paragraph, the starting purity of the antibody or antigen binding fragments of the pharmaceutical compositions is 99%.

In some embodiments, the purity of the antibody or antigen binding fragment in the pharmaceutical compositions provided herein remains stable during storage, wherein the purity changes no more than 1 percentage point over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In some embodiments, the purity of the antibody or antigen binding fragment in the pharmaceutical compositions remains stable during storage, wherein the purity changes no more than 2 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In some embodiments, the purity of the antibody or antigen binding fragment in the pharmaceutical compositions remains stable during storage, wherein the purity changes no more than 3 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In some embodiments, the purity of the antibody or antigen binding fragment in the pharmaceutical compositions remains stable during storage, wherein the purity changes no more than 4 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In some embodiments, the purity of the antibody or antigen binding fragment in the pharmaceutical compositions remains stable during storage, wherein the purity changes no more than 5 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In some embodiments of this paragraph, the starting purity of the antibody or antigen binding fragments of the pharmaceutical compositions is 95%. In some embodiments of this paragraph, the starting purity of the antibody or antigen binding fragments of the pharmaceutical compositions is 96%. In some embodiments of this paragraph, the starting purity of the antibody or antigen binding fragments of the pharmaceutical compositions is 97%. In some embodiments of this paragraph, the starting purity of the antibody or antigen binding fragments of the pharmaceutical compositions is 98%. In some embodiments of this paragraph, the starting purity of the antibody or antigen binding fragments of the pharmaceutical compositions is 99%.

In some embodiments, the purity of the antibody or antigen binding fragment in the pharmaceutical compositions provided herein remains stable during storage, wherein the purity changes no more than 1 percentage point over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40±5° C. In some embodiments, the purity of the antibody or antigen binding fragment in the pharmaceutical compositions remains stable during storage, wherein the purity changes no more than 2 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40±5° C. In some embodiments, the purity of the antibody or antigen binding fragment in the pharmaceutical compositions remains stable during storage, wherein the purity changes no more than 3 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40±5° C. In some embodiments, the purity of the antibody or antigen binding fragment in the pharmaceutical compositions remains stable during storage, wherein the purity changes no more than 4 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40±5° C. In some embodiments, the purity of the antibody or antigen binding fragment in the pharmaceutical compositions remains stable during storage, wherein the purity changes no more than 5 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40±5° C. In some embodiments of this paragraph, the starting purity of the antibody or antigen binding fragments of the pharmaceutical compositions is 95%. In some embodiments of this paragraph, the starting purity of the antibody or antigen binding fragments of the pharmaceutical compositions is 96%. In some embodiments of this paragraph, the starting purity of the antibody or antigen binding fragments of the pharmaceutical compositions is 97%. In some embodiments of this paragraph, the starting purity of the antibody or antigen binding fragments of the pharmaceutical compositions is 98%. In some embodiments of this paragraph, the starting purity of the antibody or antigen binding fragments of the pharmaceutical compositions is 99%.

In some embodiments, the purity of the antibody or antigen binding fragment in the pharmaceutical compositions provided herein remains stable during storage, wherein the purity changes no more than 1 percentage point over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In some embodiments, the purity of the antibody or antigen binding fragment in the pharmaceutical compositions remains stable during storage, wherein the purity changes no more than 2 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In some embodiments, the purity of the antibody or antigen binding fragment in the pharmaceutical compositions remains stable during storage, wherein the purity changes no more than 3 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In some embodiments, the purity of the antibody or antigen binding fragment in the pharmaceutical compositions remains stable during storage, wherein the purity changes no more than 4 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In some embodiments, the purity of the antibody or antigen binding fragment in the pharmaceutical compositions remains stable during storage, wherein the purity changes no more than 5 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In some embodiments of this paragraph, the starting purity of the antibody or antigen binding fragments of the pharmaceutical compositions is 95%. In some embodiments of this paragraph, the starting purity of the antibody or antigen binding fragments of the pharmaceutical compositions is 96%. In some embodiments of this paragraph, the starting purity of the antibody or antigen binding fragments of the pharmaceutical compositions is 97%. In some embodiments of this paragraph, the starting purity of the antibody or antigen binding fragments of the pharmaceutical compositions is 98%. In some embodiments of this paragraph, the starting purity of the antibody or antigen binding fragments of the pharmaceutical compositions is 99%.

In some embodiments, capillary isoelectric focusing (cIEF) profile of the antibody or antigen binding fragment in the pharmaceutical compositions provided herein remains stable during storage, wherein the percentage of the acidic peak, the main peak, and/or the basic peak changes no more than 1 percentage point over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In some embodiments, cIEF profile of the antibody or antigen binding fragment in the pharmaceutical compositions remains stable during storage, wherein the percentage of the acidic peak, the main peak, and/or the basic peak changes no more than 2 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In some embodiments, cIEF profile of the antibody or antigen binding fragment in the pharmaceutical compositions remains stable during storage, wherein the percentage of the acidic peak, the main peak, and/or the basic peak changes no more than 3 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In some embodiments, cIEF profile of the antibody or antigen binding fragment in the pharmaceutical compositions remains stable during storage, wherein the percentage of the acidic peak, the main peak, and/or the basic peak changes no more than 4 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In some embodiments, cIEF profile of the antibody or antigen binding fragment in the pharmaceutical compositions remains stable during storage, wherein the percentage of the acidic peak, the main peak, and/or the basic peak changes no more than 5 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In some embodiments, cIEF profile of the antibody or antigen binding fragment in the pharmaceutical compositions remains stable during storage, wherein the percentage of the acidic peak, the main peak, and/or the basic peak changes no more than 6 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In some embodiments, cIEF profile of the antibody or antigen binding fragment in the pharmaceutical compositions remains stable during storage, wherein the percentage of the acidic peak, the main peak, and/or the basic peak changes no more than 7 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In some embodiments, cIEF profile of the antibody or antigen binding fragment in the pharmaceutical compositions remains stable during storage, wherein the percentage of the acidic peak, the main peak, and/or the basic peak changes no more than 8 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In some embodiments, cIEF profile of the antibody or antigen binding fragment in the pharmaceutical compositions remains stable during storage, wherein the percentage of the acidic peak, the main peak, and/or the basic peak changes no more than 9 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In some embodiments, cIEF profile of the antibody or antigen binding fragment in the pharmaceutical compositions remains stable during storage, wherein the percentage of the acidic peak, the main peak, and/or the basic peak changes no more than 10 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In some embodiments, cIEF profile of the antibody or antigen binding fragment in the pharmaceutical compositions remains stable during storage, wherein the percentage of the acidic peak, the main peak, and/or the basic peak changes no more than 15 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In some embodiments, cIEF profile of the antibody or antigen binding fragment in the pharmaceutical compositions remains stable during storage, wherein the percentage of the acidic peak, the main peak, and/or the basic peak changes no more than 20 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In some embodiments, cIEF profile of the antibody or antigen binding fragment in the pharmaceutical compositions remains stable during storage, wherein the percentage of the acidic peak, the main peak, and/or the basic peak changes no more than 25 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 5±3° C. In some embodiments of this paragraph, the starting cIEF profile of the antibody or antigen binding fragments of the pharmaceutical compositions is 23.7% acidic peak, 67.8% main peak, and 8.6% basic peak. In some embodiments of this paragraph, the starting cIEF profile of the antibody or antigen binding fragments of the pharmaceutical compositions is 24% acidic peak, 68% main peak, and 8% basic peak. In some embodiments of this paragraph, the starting cIEF profile of the antibody or antigen binding fragments of the pharmaceutical compositions is 23% acidic peak, 68% main peak, and 9% basic peak. In some embodiments of this paragraph, the starting cIEF profile of the antibody or antigen binding fragments of the pharmaceutical compositions is 24% acidic peak, 67% main peak, and 9% basic peak.

In some embodiments, cIEF profile of the antibody or antigen binding fragment in the pharmaceutical compositions provided herein remains stable during storage, wherein the percentage of the acidic peak, the main peak, and/or the basic peak changes no more than 1 percentage point over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In some embodiments, cIEF profile of the antibody or antigen binding fragment in the pharmaceutical compositions remains stable during storage, wherein the percentage of the acidic peak, the main peak, and/or the basic peak changes no more than 2 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In some embodiments, cIEF profile of the antibody or antigen binding fragment in the pharmaceutical compositions remains stable during storage, wherein the percentage of the acidic peak, the main peak, and/or the basic peak changes no more than 3 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In some embodiments, cIEF profile of the antibody or antigen binding fragment in the pharmaceutical compositions remains stable during storage, wherein the percentage of the acidic peak, the main peak, and/or the basic peak changes no more than 4 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In some embodiments, cIEF profile of the antibody or antigen binding fragment in the pharmaceutical compositions remains stable during storage, wherein the percentage of the acidic peak, the main peak, and/or the basic peak changes no more than 5 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In some embodiments, cIEF profile of the antibody or antigen binding fragment in the pharmaceutical compositions remains stable during storage, wherein the percentage of the acidic peak, the main peak, and/or the basic peak changes no more than 6 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In some embodiments, cIEF profile of the antibody or antigen binding fragment in the pharmaceutical compositions remains stable during storage, wherein the percentage of the acidic peak, the main peak, and/or the basic peak changes no more than 7 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In some embodiments, cIEF profile of the antibody or antigen binding fragment in the pharmaceutical compositions remains stable during storage, wherein the percentage of the acidic peak, the main peak, and/or the basic peak changes no more than 8 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In some embodiments, cIEF profile of the antibody or antigen binding fragment in the pharmaceutical compositions remains stable during storage, wherein the percentage of the acidic peak, the main peak, and/or the basic peak changes no more than 9 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In some embodiments, cIEF profile of the antibody or antigen binding fragment in the pharmaceutical compositions remains stable during storage, wherein the percentage of the acidic peak, the main peak, and/or the basic peak changes no more than 10 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In some embodiments, cIEF profile of the antibody or antigen binding fragment in the pharmaceutical compositions remains stable during storage, wherein the percentage of the acidic peak, the main peak, and/or the basic peak changes no more than 15 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In some embodiments, cIEF profile of the antibody or antigen binding fragment in the pharmaceutical compositions remains stable during storage, wherein the percentage of the acidic peak, the main peak, and/or the basic peak changes no more than 20 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In some embodiments, cIEF profile of the antibody or antigen binding fragment in the pharmaceutical compositions remains stable during storage, wherein the percentage of the acidic peak, the main peak, and/or the basic peak changes no more than 25 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 25±5° C. In some embodiments of this paragraph, the starting cIEF profile of the antibody or antigen binding fragments of the pharmaceutical compositions is 23.7% acidic peak, 67.8% main peak, and 8.6% basic peak. In some embodiments of this paragraph, the starting cIEF profile of the antibody or antigen binding fragments of the pharmaceutical compositions is 24% acidic peak, 68% main peak, and 8% basic peak. In some embodiments of this paragraph, the starting cIEF profile of the antibody or antigen binding fragments of the pharmaceutical compositions is 23% acidic peak, 68% main peak, and 9% basic peak. In some embodiments of this paragraph, the starting cIEF profile of the antibody or antigen binding fragments of the pharmaceutical compositions is 24% acidic peak, 67% main peak, and 9% basic peak.

In some embodiments, cIEF profile of the antibody or antigen binding fragment in the pharmaceutical compositions provided herein remains stable during storage, wherein the percentage of the acidic peak, the main peak, and/or the basic peak changes no more than 1 percentage point over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40±5° C. In some embodiments, cIEF profile of the antibody or antigen binding fragment in the pharmaceutical compositions remains stable during storage, wherein the percentage of the acidic peak, the main peak, and/or the basic peak changes no more than 2 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40±5° C. In some embodiments, cIEF profile of the antibody or antigen binding fragment in the pharmaceutical compositions remains stable during storage, wherein the percentage of the acidic peak, the main peak, and/or the basic peak changes no more than 3 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40±5° C. In some embodiments, cIEF profile of the antibody or antigen binding fragment in the pharmaceutical compositions remains stable during storage, wherein the percentage of the acidic peak, the main peak, and/or the basic peak changes no more than 4 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40±5° C. In some embodiments, cIEF profile of the antibody or antigen binding fragment in the pharmaceutical compositions remains stable during storage, wherein the percentage of the acidic peak, the main peak, and/or the basic peak changes no more than 5 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40±5° C. In some embodiments, cIEF profile of the antibody or antigen binding fragment in the pharmaceutical compositions remains stable during storage, wherein the percentage of the acidic peak, the main peak, and/or the basic peak changes no more than 6 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40±5° C. In some embodiments, cIEF profile of the antibody or antigen binding fragment in the pharmaceutical compositions remains stable during storage, wherein the percentage of the acidic peak, the main peak, and/or the basic peak changes no more than 7 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40±5° C. In some embodiments, cIEF profile of the antibody or antigen binding fragment in the pharmaceutical compositions remains stable during storage, wherein the percentage of the acidic peak, the main peak, and/or the basic peak changes no more than 8 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40±5° C. In some embodiments, cIEF profile of the antibody or antigen binding fragment in the pharmaceutical compositions remains stable during storage, wherein the percentage of the acidic peak, the main peak, and/or the basic peak changes no more than 9 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40±5° C. In some embodiments, cIEF profile of the antibody or antigen binding fragment in the pharmaceutical compositions remains stable during storage, wherein the percentage of the acidic peak, the main peak, and/or the basic peak changes no more than 10 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40±5° C. In some embodiments, cIEF profile of the antibody or antigen binding fragment in the pharmaceutical compositions remains stable during storage, wherein the percentage of the acidic peak, the main peak, and/or the basic peak changes no more than 15 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40±5° C. In some embodiments, cIEF profile of the antibody or antigen binding fragment in the pharmaceutical compositions remains stable during storage, wherein the percentage of the acidic peak, the main peak, and/or the basic peak changes no more than 20 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40±5° C. In some embodiments, cIEF profile of the antibody or antigen binding fragment in the pharmaceutical compositions remains stable during storage, wherein the percentage of the acidic peak, the main peak, and/or the basic peak changes no more than 25 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer at 40±5° C. In some embodiments of this paragraph, the starting cIEF profile of the antibody or antigen binding fragments of the pharmaceutical compositions is 23.7% acidic peak, 67.8% main peak, and 8.6% basic peak. In some embodiments of this paragraph, the starting cIEF profile of the antibody or antigen binding fragments of the pharmaceutical compositions is 24% acidic peak, 68% main peak, and 8% basic peak. In some embodiments of this paragraph, the starting cIEF profile of the antibody or antigen binding fragments of the pharmaceutical compositions is 23% acidic peak, 68% main peak, and 9% basic peak. In some embodiments of this paragraph, the starting cIEF profile of the antibody or antigen binding fragments of the pharmaceutical compositions is 24% acidic peak, 67% main peak, and 9% basic peak.

In some embodiments, cIEF profile of the antibody or antigen binding fragment in the pharmaceutical compositions provided herein remains stable during storage, wherein the percentage of the acidic peak, the main peak, and/or the basic peak changes no more than 1 percentage point over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In some embodiments, cIEF profile of the antibody or antigen binding fragment in the pharmaceutical compositions remains stable during storage, wherein the percentage of the acidic peak, the main peak, and/or the basic peak changes no more than 2 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In some embodiments, cIEF profile of the antibody or antigen binding fragment in the pharmaceutical compositions remains stable during storage, wherein the percentage of the acidic peak, the main peak, and/or the basic peak changes no more than 3 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In some embodiments, cIEF profile of the antibody or antigen binding fragment in the pharmaceutical compositions remains stable during storage, wherein the percentage of the acidic peak, the main peak, and/or the basic peak changes no more than 4 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In some embodiments, cIEF profile of the antibody or antigen binding fragment in the pharmaceutical compositions remains stable during storage, wherein the percentage of the acidic peak, the main peak, and/or the basic peak changes no more than 5 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In some embodiments, cIEF profile of the antibody or antigen binding fragment in the pharmaceutical compositions remains stable during storage, wherein the percentage of the acidic peak, the main peak, and/or the basic peak changes no more than 6 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In some embodiments, cIEF profile of the antibody or antigen binding fragment in the pharmaceutical compositions remains stable during storage, wherein the percentage of the acidic peak, the main peak, and/or the basic peak changes no more than 7 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In some embodiments, cIEF profile of the antibody or antigen binding fragment in the pharmaceutical compositions remains stable during storage, wherein the percentage of the acidic peak, the main peak, and/or the basic peak changes no more than 8 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In some embodiments, cIEF profile of the antibody or antigen binding fragment in the pharmaceutical compositions remains stable during storage, wherein the percentage of the acidic peak, the main peak, and/or the basic peak changes no more than 9 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In some embodiments, cIEF profile of the antibody or antigen binding fragment in the pharmaceutical compositions remains stable during storage, wherein the percentage of the acidic peak, the main peak, and/or the basic peak changes no more than 10 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In some embodiments, cIEF profile of the antibody or antigen binding fragment in the pharmaceutical compositions remains stable during storage, wherein the percentage of the acidic peak, the main peak, and/or the basic peak changes no more than 15 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In some embodiments, cIEF profile of the antibody or antigen binding fragment in the pharmaceutical compositions remains stable during storage, wherein the percentage of the acidic peak, the main peak, and/or the basic peak changes no more than 20 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In some embodiments, cIEF profile of the antibody or antigen binding fragment in the pharmaceutical compositions remains stable during storage, wherein the percentage of the acidic peak, the main peak, and/or the basic peak changes no more than 25 percentage points over 1 week, 2 weeks, 4 weeks, 8 weeks, 2 months, 3 months, 4 months, 5 months, 6 months, 26 weeks, 7 months, 8 months, 9 months, 10 months, 11 months, 12 months, 14 months, 24 months, or longer in the presence of 0.1-5.0 ppm H2O2 at 25±5° C. In some embodiments of this paragraph, the starting cIEF profile of the antibody or antigen binding fragments of the pharmaceutical compositions is 23.7% acidic peak, 67.8% main peak, and 8.6% basic peak. In some embodiments of this paragraph, the starting cIEF profile of the antibody or antigen binding fragments of the pharmaceutical compositions is 24% acidic peak, 68% main peak, and 8% basic peak. In some embodiments of this paragraph, the starting cIEF profile of the antibody or antigen binding fragments of the pharmaceutical compositions is 23% acidic peak, 68% main peak, and 9% basic peak. In some embodiments of this paragraph, the starting cIEF profile of the antibody or antigen binding fragments of the pharmaceutical compositions is 24% acidic peak, 67% main peak, and 9% basic peak.

In some embodiments, the pharmaceutical compositions provided herein have a stable pH value, wherein the pH changes no more than 0.1 unit after agitation (e.g. 100 RPM) for up to 1 day, agitation (e.g. 100 RPM) for up to 3 days, or vigorous agitation (e.g. 300 RPM) for up to 3 days. In some embodiments, the pharmaceutical compositions have a stable pH value, wherein the pH changes no more than 0.2 unit after agitation (e.g. 100 RPM) for up to 1 day, agitation (e.g. 100 RPM) for up to 3 days, or vigorous agitation (e.g. 300 RPM) for up to 3 days. In some embodiments, the pharmaceutical compositions have a stable pH value, wherein the pH changes no more than 0.1 unit after up to 3 cycles, up to 5 cycles, or up to 10 cycles of freeze and thaw from −80° C. to room temperature. In some embodiments, the pharmaceutical compositions have a stable pH value, wherein the pH changes no more than 0.2 unit after up to 3 cycles, up to 5 cycles, or up to 10 cycles of freeze and thaw from −80° C. to room temperature. In some embodiments of this paragraph, the starting pH of the pharmaceutical compositions is 5.0. In some embodiments of this paragraph, the starting pH of the pharmaceutical compositions is 6.0.

In some embodiments, the pharmaceutical compositions provided herein have a stable pI value, wherein the pI changes no more than 0.1 unit after agitation (e.g. 100 RPM) for up to 1 day, agitation (e.g. 100 RPM) for up to 3 days, or vigorous agitation (e.g. 300 RPM) for up to 3 days. In some embodiments, the pharmaceutical compositions have a stable pI value, wherein the pI changes no more than 0.2 unit after agitation (e.g. 100 RPM) for up to 1 day, agitation (e.g. 100 RPM) for up to 3 days, or vigorous agitation (e.g. 300 RPM) for up to 3 days. In some embodiments, the pharmaceutical compositions have a stable pI value, wherein the pI changes no more than 0.1 unit after up to 3 cycles, up to 5 cycles, or up to 10 cycles of freeze and thaw from −80° C. to room temperature. In some embodiments, the pharmaceutical compositions have a stable pI value, wherein the pI changes no more than 0.2 unit after up to 3 cycles, up to 5 cycles, or up to 10 cycles of freeze and thaw from −80° C. to room temperature. In some embodiments of this paragraph, the starting pI of the antibody or antigen binding fragments of the pharmaceutical compositions is 9.0.

In some embodiments, the pharmaceutical compositions provided herein have a stable concentration of the antibody or antigen binding fragment, wherein the concentration of the antibody or antigen binding fragment changes no more than 10 mg/ml after agitation (e.g. 100 RPM) for up to 1 day, agitation (e.g. 100 RPM) for up to 3 days, or vigorous agitation (e.g. 300 RPM) for up to 3 days. In some embodiments, the pharmaceutical compositions have a stable concentration of the antibody or antigen binding fragment, wherein the concentration of the antibody or antigen binding fragment changes no more than 5 mg/ml after agitation (e.g. 100 RPM) for up to 1 day, agitation (e.g. 100 RPM) for up to 3 days, or vigorous agitation (e.g. 300 RPM) for up to 3 days. In some embodiments, the pharmaceutical compositions have a stable concentration of the antibody or antigen binding fragment, wherein the concentration of the antibody or antigen binding fragment changes no more than 10% after agitation (e.g. 100 RPM) for up to 1 day, agitation (e.g. 100 RPM) for up to 3 days, or vigorous agitation (e.g. 300 RPM) for up to 3 days. In some embodiments, the pharmaceutical compositions have a stable concentration of the antibody or antigen binding fragment, wherein the concentration of the antibody or antigen binding fragment changes no more than 5% after agitation (e.g. 100 RPM) for up to 1 day, agitation (e.g. 100 RPM) for up to 3 days, or vigorous agitation (e.g. 300 RPM) for up to 3 days. In some embodiments, the pharmaceutical compositions have a stable concentration of the antibody or antigen binding fragment, wherein the concentration of the antibody or antigen binding fragment changes no more than 4% after agitation (e.g. 100 RPM) for up to 1 day, agitation (e.g. 100 RPM) for up to 3 days, or vigorous agitation (e.g. 300 RPM) for up to 3 days. In some embodiments, the pharmaceutical compositions have a stable concentration of the antibody or antigen binding fragment, wherein the concentration of the antibody or antigen binding fragment changes no more than 3% after agitation (e.g. 100 RPM) for up to 1 day, agitation (e.g. 100 RPM) for up to 3 days, or vigorous agitation (e.g. 300 RPM) for up to 3 days. In some embodiments, the pharmaceutical compositions have a stable concentration of the antibody or antigen binding fragment, wherein the concentration of the antibody or antigen binding fragment changes no more than 2% after agitation (e.g. 100 RPM) for up to 1 day, agitation (e.g. 100 RPM) for up to 3 days, or vigorous agitation (e.g. 300 RPM) for up to 3 days. In some embodiments, the pharmaceutical compositions have a stable concentration of the antibody or antigen binding fragment, wherein the concentration of the antibody or antigen binding fragment changes no more than 1% after agitation (e.g. 100 RPM) for up to 1 day, agitation (e.g. 100 RPM) for up to 3 days, or vigorous agitation (e.g. 300 RPM) for up to 3 days. In some embodiments, the pharmaceutical compositions have a stable concentration of the antibody or antigen binding fragment, wherein the concentration of the antibody or antigen binding fragment changes no more than 10 mg/ml after up to 3 cycles, up to 5 cycles, or up to 10 cycles of freeze and thaw from −80° C. to room temperature. In some embodiments, the pharmaceutical compositions have a stable concentration of the antibody or antigen binding fragment, wherein the concentration of the antibody or antigen binding fragment changes no more than 5 mg/ml after up to 3 cycles, up to 5 cycles, or up to 10 cycles of freeze and thaw from −80° C. to room temperature. In some embodiments, the pharmaceutical compositions have a stable concentration of the antibody or antigen binding fragment, wherein the concentration of the antibody or antigen binding fragment changes no more than 10% after up to 3 cycles, up to 5 cycles, or up to 10 cycles of freeze and thaw from −80° C. to room temperature. In some embodiments, the pharmaceutical compositions have a stable concentration of the antibody or antigen binding fragment, wherein the concentration of the antibody or antigen binding fragment changes no more than 5% after up to 3 cycles, up to 5 cycles, or up to 10 cycles of freeze and thaw from −80° C. to room temperature. In some embodiments, the pharmaceutical compositions have a stable concentration of the antibody or antigen binding fragment, wherein the concentration of the antibody or antigen binding fragment changes no more than 4% after up to 3 cycles, up to 5 cycles, or up to 10 cycles of freeze and thaw from −80° C. to room temperature. In some embodiments, the pharmaceutical compositions have a stable concentration of the antibody or antigen binding fragment, wherein the concentration of the antibody or antigen binding fragment changes no more than 3% after up to 3 cycles, up to 5 cycles, or up to 10 cycles of freeze and thaw from −80° C. to room temperature. In some embodiments, the pharmaceutical compositions have a stable concentration of the antibody or antigen binding fragment, wherein the concentration of the antibody or antigen binding fragment changes no more than 2% after up to 3 cycles, up to 5 cycles, or up to 10 cycles of freeze and thaw from −80° C. to room temperature. In some embodiments, the pharmaceutical compositions have a stable concentration of the antibody or antigen binding fragment, wherein the concentration of the antibody or antigen binding fragment changes no more than 1% after up to 3 cycles, up to 5 cycles, or up to 10 cycles of freeze and thaw from −80° C. to room temperature. In some embodiments of this paragraph, the starting concentration of the antibody or antigen binding fragments of the pharmaceutical compositions provided herein is about 200 mg/ml. In some embodiments of this paragraph, the starting concentration of the antibody or antigen binding fragments of the pharmaceutical compositions provided herein is at least 200 mg/ml.

In some embodiments, the purity of the antibody or antigen binding fragment in the pharmaceutical compositions provided herein remains stable during storage, wherein the purity changes no more than 1 percentage point after agitation (e.g. 100 RPM) for up to 1 day, agitation (e.g. 100 RPM) for up to 3 days, or vigorous agitation (e.g. 300 RPM) for up to 3 days. In some embodiments, the purity of the antibody or antigen binding fragment in the pharmaceutical compositions remains stable during storage, wherein the purity changes no more than 2 percentage points after agitation (e.g. 100 RPM) for up to 1 day, agitation (e.g. 100 RPM) for up to 3 days, or vigorous agitation (e.g. 300 RPM) for up to 3 days. In some embodiments, the purity of the antibody or antigen binding fragment in the pharmaceutical compositions remains stable during storage, wherein the purity changes no more than 3 percentage points after agitation (e.g. 100 RPM) for up to 1 day, agitation (e.g. 100 RPM) for up to 3 days, or vigorous agitation (e.g. 300 RPM) for up to 3 days. In some embodiments, the purity of the antibody or antigen binding fragment in the pharmaceutical compositions remains stable during storage, wherein the purity changes no more than 4 percentage points after agitation (e.g. 100 RPM) for up to 1 day, agitation (e.g. 100 RPM) for up to 3 days, or vigorous agitation (e.g. 300 RPM) for up to 3 days. In some embodiments, the purity of the antibody or antigen binding fragment in the pharmaceutical compositions remains stable during storage, wherein the purity changes no more than 5 percentage points after agitation (e.g. 100 RPM) for up to 1 day, agitation (e.g. 100 RPM) for up to 3 days, or vigorous agitation (e.g. 300 RPM) for up to 3 days. In some embodiments of this paragraph, the starting purity of the antibody or antigen binding fragments in the pharmaceutical compositions is 95%. In some embodiments of this paragraph, the starting purity of the antibody or antigen binding fragments in the pharmaceutical compositions is 96%. In some embodiments of this paragraph, the starting purity of the antibody or antigen binding fragments in the pharmaceutical compositions is 97%. In some embodiments of this paragraph, the starting purity of the antibody or antigen binding fragments in the pharmaceutical compositions is 98%. In some embodiments of this paragraph, the starting purity of the antibody or antigen binding fragments in the pharmaceutical compositions is 99%.

In some embodiments, the purity of the antibody or antigen binding fragment in the pharmaceutical compositions provided herein remains stable during storage, wherein the purity changes no more than 1 percentage point after up to 3 cycles, up to 5 cycles, or up to 10 cycles of freeze and thaw from −80° C. to room temperature. In some embodiments, the purity of the antibody or antigen binding fragment in the pharmaceutical compositions remains stable during storage, wherein the purity changes no more than 2 percentage points after up to 3 cycles, up to 5 cycles, or up to 10 cycles of freeze and thaw from −80° C. to room temperature. In some embodiments, the purity of the antibody or antigen binding fragment in the pharmaceutical compositions remains stable during storage, wherein the purity changes no more than 3 percentage points after up to 3 cycles, up to 5 cycles, or up to 10 cycles of freeze and thaw from −80° C. to room temperature. In some embodiments, the purity of the antibody or antigen binding fragment in the pharmaceutical compositions remains stable during storage, wherein the purity changes no more than 4 percentage points after up to 3 cycles, up to 5 cycles, or up to 10 cycles of freeze and thaw from −80° C. to room temperature. In some embodiments, the purity of the antibody or antigen binding fragment in the pharmaceutical compositions remains stable during storage, wherein the purity changes no more than 5 percentage points after up to 3 cycles, up to 5 cycles, or up to 10 cycles of freeze and thaw from −80° C. to room temperature. In some embodiments of this paragraph, the starting purity of the antibody or antigen binding fragments in the pharmaceutical compositions is 95%. In some embodiments of this paragraph, the starting purity of the antibody or antigen binding fragments in the pharmaceutical compositions is 96%. In some embodiments of this paragraph, the starting purity of the antibody or antigen binding fragments in the pharmaceutical compositions is 97%. In some embodiments of this paragraph, the starting purity of the antibody or antigen binding fragments in the pharmaceutical compositions is 98%. In some embodiments of this paragraph, the starting purity of the antibody or antigen binding fragments in the pharmaceutical compositions is 99%.

In some embodiments, cIEF profile of the antibody or antigen binding fragment in the pharmaceutical compositions provided herein remains stable during storage, wherein the percentage of the acidic peak, the main peak, and/or the basic peak changes no more than 1 percentage point after agitation (e.g. 100 RPM) for up to 1 day, agitation (e.g. 100 RPM) for up to 3 days, or vigorous agitation (e.g. 300 RPM) for up to 3 days. In some embodiments, cIEF profile of the antibody or antigen binding fragment in the pharmaceutical compositions remains stable during storage, wherein the percentage of the acidic peak, the main peak, and/or the basic peak changes no more than 2 percentage points after agitation (e.g. 100 RPM) for up to 1 day, agitation (e.g. 100 RPM) for up to 3 days, or vigorous agitation (e.g. 300 RPM) for up to 3 days. In some embodiments, cIEF profile of the antibody or antigen binding fragment in the pharmaceutical compositions remains stable during storage, wherein the percentage of the acidic peak, the main peak, and/or the basic peak changes no more than 3 percentage points after agitation (e.g. 100 RPM) for up to 1 day, agitation (e.g. 100 RPM) for up to 3 days, or vigorous agitation (e.g. 300 RPM) for up to 3 days. In some embodiments, cIEF profile of the antibody or antigen binding fragment in the pharmaceutical compositions remains stable during storage, wherein the percentage of the acidic peak, the main peak, and/or the basic peak changes no more than 4 percentage points after agitation (e.g. 100 RPM) for up to 1 day, agitation (e.g. 100 RPM) for up to 3 days, or vigorous agitation (e.g. 300 RPM) for up to 3 days. In some embodiments, cIEF profile of the antibody or antigen binding fragment in the pharmaceutical compositions remains stable during storage, wherein the percentage of the acidic peak, the main peak, and/or the basic peak changes no more than 5 percentage points after agitation (e.g. 100 RPM) for up to 1 day, agitation (e.g. 100 RPM) for up to 3 days, or vigorous agitation (e.g. 300 RPM) for up to 3 days. In some embodiments, cIEF profile of the antibody or antigen binding fragment in the pharmaceutical compositions remains stable during storage, wherein the percentage of the acidic peak, the main peak, and/or the basic peak changes no more than 6 percentage points after agitation (e.g. 100 RPM) for up to 1 day, agitation (e.g. 100 RPM) for up to 3 days, or vigorous agitation (e.g. 300 RPM) for up to 3 days. In some embodiments, cIEF profile of the antibody or antigen binding fragment in the pharmaceutical compositions remains stable during storage, wherein the percentage of the acidic peak, the main peak, and/or the basic peak changes no more than 7 percentage points after agitation (e.g. 100 RPM) for up to 1 day, agitation (e.g. 100 RPM) for up to 3 days, or vigorous agitation (e.g. 300 RPM) for up to 3 days. In some embodiments, cIEF profile of the antibody or antigen binding fragment in the pharmaceutical compositions remains stable during storage, wherein the percentage of the acidic peak, the main peak, and/or the basic peak changes no more than 8 percentage points after agitation (e.g. 100 RPM) for up to 1 day, agitation (e.g. 100 RPM) for up to 3 days, or vigorous agitation (e.g. 300 RPM) for up to 3 days. In some embodiments, cIEF profile of the antibody or antigen binding fragment in the pharmaceutical compositions remains stable during storage, wherein the percentage of the acidic peak, the main peak, and/or the basic peak changes no more than 9 percentage points after agitation (e.g. 100 RPM) for up to 1 day, agitation (e.g. 100 RPM) for up to 3 days, or vigorous agitation (e.g. 300 RPM) for up to 3 days. In some embodiments, cIEF profile of the antibody or antigen binding fragment in the pharmaceutical compositions remains stable during storage, wherein the percentage of the acidic peak, the main peak, and/or the basic peak changes no more than 10 percentage points after agitation (e.g. 100 RPM) for up to 1 day, agitation (e.g. 100 RPM) for up to 3 days, or vigorous agitation (e.g. 300 RPM) for up to 3 days. In some embodiments of this paragraph, the starting cIEF profile of the antibody or antigen binding fragments of the pharmaceutical compositions is 23.7% acidic peak, 67.8% main peak, and 8.6% basic peak. In some embodiments of this paragraph, the starting cIEF profile of the antibody or antigen binding fragments of the pharmaceutical compositions is 24% acidic peak, 68% main peak, and 8% basic peak. In some embodiments of this paragraph, the starting cIEF profile of the antibody or antigen binding fragments of the pharmaceutical compositions is 23% acidic peak, 68% main peak, and 9% basic peak. In some embodiments of this paragraph, the starting cIEF profile of the antibody or antigen binding fragments of the pharmaceutical compositions is 24% acidic peak, 67% main peak, and 9% basic peak.

In some embodiments, cIEF profile of the antibody or antigen binding fragment in the pharmaceutical compositions provided herein remains stable during storage, wherein the percentage of the acidic peak, the main peak, and/or the basic peak changes no more than 1 percentage point after up to 3 cycles, up to 5 cycles, or up to 10 cycles of freeze and thaw from −80° C. to room temperature. In some embodiments, cIEF profile of the antibody or antigen binding fragment in the pharmaceutical compositions remains stable during storage, wherein the percentage of the acidic peak, the main peak, and/or the basic peak changes no more than 2 percentage points after up to 3 cycles, up to 5 cycles, or up to 10 cycles of freeze and thaw from −80° C. to room temperature. In some embodiments, cIEF profile of the antibody or antigen binding fragment in the pharmaceutical compositions remains stable during storage, wherein the percentage of the acidic peak, the main peak, and/or the basic peak changes no more than 3 percentage points after up to 3 cycles, up to 5 cycles, or up to 10 cycles of freeze and thaw from −80° C. to room temperature. In some embodiments, cIEF profile of the antibody or antigen binding fragment in the pharmaceutical compositions remains stable during storage, wherein the percentage of the acidic peak, the main peak, and/or the basic peak changes no more than 4 percentage points after up to 3 cycles, up to 5 cycles, or up to 10 cycles of freeze and thaw from −80° C. to room temperature. In some embodiments, cIEF profile of the antibody or antigen binding fragment in the pharmaceutical compositions remains stable during storage, wherein the percentage of the acidic peak, the main peak, and/or the basic peak changes no more than 5 percentage points after up to 3 cycles, up to 5 cycles, or up to 10 cycles of freeze and thaw from −80° C. to room temperature. In some embodiments, cIEF profile of the antibody or antigen binding fragment in the pharmaceutical compositions remains stable during storage, wherein the percentage of the acidic peak, the main peak, and/or the basic peak changes no more than 6 percentage points after up to 3 cycles, up to 5 cycles, or up to 10 cycles of freeze and thaw from −80° C. to room temperature. In some embodiments, cIEF profile of the antibody or antigen binding fragment in the pharmaceutical compositions remains stable during storage, wherein the percentage of the acidic peak, the main peak, and/or the basic peak changes no more than 7 percentage points after up to 3 cycles, up to 5 cycles, or up to 10 cycles of freeze and thaw from −80° C. to room temperature. In some embodiments, cIEF profile of the antibody or antigen binding fragment in the pharmaceutical compositions remains stable during storage, wherein the percentage of the acidic peak, the main peak, and/or the basic peak changes no more than 8 percentage points after up to 3 cycles, up to 5 cycles, or up to 10 cycles of freeze and thaw from −80° C. to room temperature. In some embodiments, cIEF profile of the antibody or antigen binding fragment in the pharmaceutical compositions remains stable during storage, wherein the percentage of the acidic peak, the main peak, and/or the basic peak changes no more than 9 percentage points after up to 3 cycles, up to 5 cycles, or up to 10 cycles of freeze and thaw from −80° C. to room temperature. In some embodiments, cIEF profile of the antibody or antigen binding fragment in the pharmaceutical compositions remains stable during storage, wherein the percentage of the acidic peak, the main peak, and/or the basic peak changes no more than 10 percentage points after up to 3 cycles, up to 5 cycles, or up to 10 cycles of freeze and thaw from −80° C. to room temperature. In some embodiments of this paragraph, the starting cIEF profile of the antibody or antigen binding fragments of the pharmaceutical compositions is 23.7% acidic peak, 67.8% main peak, and 8.6% basic peak. In some embodiments of this paragraph, the starting cIEF profile of the antibody or antigen binding fragments of the pharmaceutical compositions is 24% acidic peak, 68% main peak, and 8% basic peak. In some embodiments of this paragraph, the starting cIEF profile of the antibody or antigen binding fragments of the pharmaceutical compositions is 23% acidic peak, 68% main peak, and 9% basic peak. In some embodiments of this paragraph, the starting cIEF profile of the antibody or antigen binding fragments of the pharmaceutical compositions is 24% acidic peak, 67% main peak, and 9% basic peak.

Additionally, for example, in some embodiments, the pharmaceutical composition of anti-CD30L is characterized by a turbidity less than 12 Nephelometric Turbidity Units (NTU) when the anti-CD30L is at a concentration greater than about 100 mg/mL e.g., about 150 mg/mL to about 300 mg/mL, about 150 mg/mL to about 200 mg/mL, about 150 mg/mL to about 225 mg/mL, or about 150 mg/mL to about 250 mg/mL. In some cases, the anti-CD30L is selected from the group consisting of anti-CD30L antibodies or antigen binding fragments provided in Section 4.1. In some embodiments, the pharmaceutical composition of anti-CD30L is characterized by a turbidity less than 12 Nephelometric Turbidity Units (NTU) when the anti-CD30L is at a concentration greater than at least about 150 mg/mL. In some embodiments, the pharmaceutical composition of anti-CD30L is characterized by a turbidity less than 12 Nephelometric Turbidity Units (NTU) when the anti-CD30L is at a concentration greater than at least about 160 mg/mL. In some embodiments, the pharmaceutical composition of anti-CD30L is characterized by a turbidity less than 12 Nephelometric Turbidity Units (NTU) when the anti-CD30L is at a concentration greater than at least about 170 mg/mL. In some embodiments, the pharmaceutical composition of anti-CD30L is characterized by a turbidity less than 12 Nephelometric Turbidity Units (NTU) when the anti-CD30L is at a concentration greater than at least about 180 mg/mL. In some embodiments, the pharmaceutical composition of anti-CD30L is characterized by a turbidity less than 12 Nephelometric Turbidity Units (NTU) when the anti-CD30L is at a concentration greater than at least about 190 mg/mL. In some embodiments, the pharmaceutical composition of anti-CD30L is characterized by a turbidity less than 12 Nephelometric Turbidity Units (NTU) when the anti-CD30L is at a concentration of about 150 mg/mL to about 250 mg/mL. Less than 12 NTU may include about 1, 2, 3, 4, or 5 NTU to about 12 NTU.

The composition provided herein may have a viscosity of no more than or about 20 centipoise (cP). The composition provided herein may have a viscosity of no more than or about 19 centipoise (cP). The composition provided herein may have a viscosity of no more than or about 18 centipoise (cP). The composition provided herein may have a viscosity of no more than or about 17 centipoise (cP). The composition provided herein may have a viscosity of no more than or about 16 centipoise (cP). The composition may have a viscosity of no more than or about 15 centipoise (cP). The composition provided herein may have a viscosity of no more than or about 14 centipoise (cP). The composition provided herein may have a viscosity of no more than or about 13 centipoise (cP). The composition provided herein may have a viscosity of no more than or about 12 centipoise (cP). The composition provided herein may have a viscosity of no more than or about 11 centipoise (cP). The composition provided herein may have a viscosity of no more than or about 10 centipoise (cP). The composition provided herein may have a viscosity of no more than or about 9 centipoise (cP). The composition provided herein may have a viscosity of no more than or about 8 centipoise (cP). The composition provided herein may have a viscosity of no more than or about 7 centipoise (cP). The composition provided herein may have a viscosity of no more than or about 6 centipoise (cP). The composition provided herein may have a viscosity of no more than or about 5 centipoise (cP). The composition may have a viscosity of no more than or about 4 centipoise (cP). For instance, the composition has a viscosity of no more than or about 20, 19, 18, 17, 16, 15, 14, 13, 12, 11, 10, 9, 8, 7, 6, 5, 4, 3, or 2 cP. The composition may have a viscosity of at least about 1, 2 or 3 cP. Further example viscosities include about 1 cP to about 2 cP, about 1 cP to about 3 cP, about 1 cP to about 4 cP, about 1 cP to about 5 cP, about 1 cP to about 6 cP, about 1 cP to about 7 cP, about 1 cP to about 8 cP, about 1 cP to about 9 cP, about 1 cP to about 10 cP, about 1 cP to about 11 cP, about 1 cP to about 12 cP, about 1 cP to about 13 cP, about 1 cP to about 14 cP, about 1 cP to about 15 cP, about 1 cP to about 16 cP, about 1 cP to about 17 cP, about 1 cP to about 18 cP, about 1 cP to about 19 cP, about 1 cP to about 20 cP, about 2 cP to about 5 cP, about 2 cP to about 6 cP, about 2 cP to about 7 cP, about 2 cP to about 8 cP, about 2 cP to about 9 cP, about 2 cP to about 10 cP, about 2 cP to about 11 cP, about 2 cP to about 12 cP, about 2 cP to about 13 cP, about 2 cP to about 14 cP, about 2 cP to about 15 cP, about 2 cP to about 16 cP, about 2 cP to about 17 cP, about 2 cP to about 18 cP, about 2 cP to about 19 cP, about 2 cP to about 20 cP, about 3 cP to about 5 cP, about 3 cP to about 6 cP, about 3 cP to about 7 cP, about 3 cP to about 8 cP, about 3 cP to about 9 cP, about 3 cP to about 10 cP, about 3 cP to about 11 cP, about 3 cP to about 12 cP, about 3 cP to about 13 cP, about 3 cP to about 14 cP, about 3 cP to about 15 cP, about 3 cP to about 16 cP, about 3 cP to about 17 cP, about 3 cP to about 18 cP, about 3 cP to about 19 cP, about cP to about 20 cP, about 4 cP to about 5 cP, about 4 cP to about 6 cP, about 4 cP to about 7 cP, about 4 cP to about 8 cP, about 4 cP to about 9 cP, or about 4 cP to about 10 cP, about 4 cP to about 11 cP, about 4 cP to about 12 cP, about 4 cP to about 13 cP, about 4 cP to about 14 cP, about 4 cP to about 15 cP, about 4 cP to about 16 cP, about 4 cP to about 17 cP, about 4 cP to about 18 cP, about 4 cP to about 19 cP, about 4 cP to about 20 cP, about 5 cP to about 10 cP, about 5 cP to about 11 cP, about 5 cP to about 12 cP, about 5 cP to about 13 cP, about 5 cP to about 14 cP, about 5 cP to about 15 cP, about 5 cP to about 16 cP, about 5 cP to about 17 cP, about 5 cP to about 18 cP, about 5 cP to about 19 cP, about 5 cP to about 20 cP, about 6 cP to about 10 cP, about 6 cP to about 11 cP, about 6 cP to about 12 cP, about 6 cP to about 13 cP, about 6 cP to about 14 cP, about 6 cP to about 15 cP, about 6 cP to about 16 cP, about 6 cP to about 17 cP, about 6 cP to about 18 cP, about 6 cP to about 19 cP, about 6 cP to about 20 cP, about 7 cP to about 10 cP, about 7 cP to about 11 cP, about 7 cP to about 12 cP, about 7 cP to about 13 cP, about 7 cP to about 14 cP, about 7 cP to about 15 cP, about 7 cP to about 16 cP, about 7 cP to about 17 cP, about 7 cP to about 18 cP, about 7 cP to about 19 cP, about 7 cP to about 20 cP, about 8 cP to about 10 cP, about 8 cP to about 11 cP, about 8 cP to about 12 cP, about 8 cP to about 13 cP, about 8 cP to about 14 cP, about 8 cP to about 15 cP, about 8 cP to about 16 cP, about 8 cP to about 17 cP, about 8 cP to about 18 cP, about 8 cP to about 19 cP, or about 8 cP to about 20 cP. In some embodiments, a centipoise as used herein is a millipascal-second (mPa·s).

In certain embodiments, the pharmaceutical composition has a volume suitable for injection, such as via subcutaneous administration. In some embodiments, the total volume of the composition may be less than or equal to about 2.5 mL. In some embodiments, the total volume of the composition is less than about 2 mL, less than about or equal to about 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, or 0.8 mL. Antibody therapeutics suitable for injection and/or administration are important to realizing full therapeutic potential. However, administration is generally restricted by volume, for instance, when the therapeutic is delivered subcutaneously. This, in turn, elucidates the importance developing of high concentration antibody formulations of greater than, for example in some cases, 100 mg/ml, 150 mg/ml, or 200 mg/ml. Problems associated with antibody development include high solution viscosity and opalescence, which are commonly encountered during the development of high-concentration (e.g., greater than 100 mg/ml, 150 mg/ml, or 200 mg/ml). Both viscosity and opalescence impact antibody developability broadly, affecting manufacturability, stability, and delivery. High solution viscosities (e.g., greater than 30 mPa-s) cause limiting back-pressures in ultrafiltration/diafiltration during the antibody concentration unit operation. Similarly, viscous antibody solutions also result in forbidding or incompatible injection forces when administering via injection, including via patient friendly autoinjectors. In effect, solution viscosity can be a determining factor for the maximum antibody dose possible via injection. Solution opalescence in therapeutic antibodies can be equally problematic as opalescence can indicate predisposition for liquid-liquid phase separation, precipitation, or aggregation. Further difficulty may occur with blinding of subcutaneous placebo.

The anti-CD30L antibodies or antigen binding fragments provided herein demonstrate advantageous viscosity and aggregation properties at high antibody concentrations (e.g., greater than about 100, 125, 150, 160, 170, 180, 190, or 200 mg/mL). Notably, anti-CD30L antibodies or antigen binding fragments provided herein are characterized by low viscosity (e.g., less than 20 mPa-s) and low aggregation (e.g., less than 5% high molecular weight species and as provided elsewhere in this Section 4.5.7) at high concentrations.

For example, in some embodiments, the anti-CD30L antibodies or antigen binding fragments are characterized by a viscosity of no more than or about 30, 20, 19, 18, 17, 16, 15, 14, 13, 12, 11, 10, 9, 8, 7, 6, or 5 mPa-s at a concentration greater than about 100 mg/mL, e.g., about 150 mg/mL to about 300 mg/mL, about 150 mg/mL to about 200 mg/mL, about 150 mg/mL to about 225 mg/mL, or about 150 mg/mL to about 250 mg/mL. In some embodiments, the anti-CD30L antibody or antigen binding fragment is selected from the group consisting of anti-CD30L antibodies or antigen binding fragments provided in Section 4.1. In some specific embodiment, the antibody comprises a CDR-H1 comprising the amino acid sequence set forth in any one of SEQ ID NOs: 110-119, 629, 636, and 712-723, a CDR-H2 comprising the amino acid sequence set forth in any one of SEQ ID NOs: 150-159, 643, 650, and 724-735, a CDR-H3 comprising the amino acid sequence set forth in any one of SEQ ID NOs: 190-199, 657, 664, and 736-743, a CDR-L1 comprising the amino acid sequence set forth in any one of SEQ ID NOs: 310-319, 671, 678, and 744-751, a CDR-L2 comprising the amino acid sequence set forth in any one of SEQ ID NOs: 350-359, 685, 692, and 752-759, and a CDR-L3 comprising the amino acid sequence set forth in any one of SEQ ID NOs: 390-399, 699, 706, and 760-765. For instance, the pharmaceutical composition has a viscosity of less than or about 20, 19, 18, 17, 16, 15, 14, 13, 12, 11, 10, 9, 8, 7, 6, 5, 4, 3, or 2 cP. The pharmaceutical composition may have a viscosity of at least about 1, 2 or 3 cP. Further example viscosities include about 1 cP to about 5 cP, about 1 cP to about 6 cP, about 1 cP to about 7 cP, about 1 cP to about 8 cP, about 1 cP to about 9 cP, about 1 cP to about 10 cP, about 1 cP to about 11 cP, about 1 cP to about 12 cP, about 1 cP to about 13 cP, about 1 cP to about 14 cP, about 1 cP to about 15 cP, about 1 cP to about 16 cP, about 1 cP to about 17 cP, about 1 cP to about 18 cP, about 1 cP to about 19 cP, about 1 cP to about 20 cP, about 2 cP to about 5 cP, about 2 cP to about 6 cP, about 2 cP to about 7 cP, about 2 cP to about 8 cP, about 2 cP to about 9 cP, about 2 cP to about 10 cP, about 2 cP to about 11 cP, about 2 cP to about 12 cP, about 2 cP to about 13 cP, about 2 cP to about 14 cP, about 2 cP to about 15 cP, about 2 cP to about 16 cP, about 2 cP to about 17 cP, about 2 cP to about 18 cP, about 2 cP to about 19 cP, about 2 cP to about 20 cP, about 3 cP to about 5 cP, about 3 cP to about 6 cP, about 3 cP to about 7 cP, about 3 cP to about 8 cP, about 3 cP to about 9 cP, about 3 cP to about 10 cP, about 3 cP to about 11 cP, about 3 cP to about 12 cP, about 3 cP to about 13 cP, about 3 cP to about 14 cP, about 3 cP to about 15 cP, about 3 cP to about 16 cP, about 3 cP to about 17 cP, about 3 cP to about 18 cP, about 3 cP to about 19 cP, about cP to about 20 cP, about 4 cP to about 5 cP, about 4 cP to about 6 cP, about 4 cP to about 7 cP, about 4 cP to about 8 cP, about 4 cP to about 9 cP, about 4 cP to about 10 cP, about 4 cP to about 11 cP, about 4 cP to about 12 cP, about 4 cP to about 13 cP, about 4 cP to about 14 cP, about 4 cP to about 15 cP, about 4 cP to about 16 cP, about 4 cP to about 17 cP, about 4 cP to about 18 cP, about 4 cP to about 19 cP, about 4 cP to about 20 cP, about 5 cP to about 10 cP, about 5 cP to about 11 cP, about 5 cP to about 12 cP, about 5 cP to about 13 cP, about 5 cP to about 14 cP, about 5 cP to about 15 cP, about 5 cP to about 16 cP, about 5 cP to about 17 cP, about 5 cP to about 18 cP, about 5 cP to about 19 cP, about 5 cP to about 20 cP, about 6 cP to about 10 cP, about 6 cP to about 11 cP, about 6 cP to about 12 cP, about 6 cP to about 13 cP, about 6 cP to about 14 cP, about 6 cP to about 15 cP, about 6 cP to about 16 cP, about 6 cP to about 17 cP, about 6 cP to about 18 cP, about 6 cP to about 19 cP, about 6 cP to about 20 cP, about 7 cP to about 10 cP, about 7 cP to about 11 cP, about 7 cP to about 12 cP, about 7 cP to about 13 cP, about 7 cP to about 14 cP, about 7 cP to about 15 cP, about 7 cP to about 16 cP, about 7 cP to about 17 cP, about 7 cP to about 18 cP, about 7 cP to about 19 cP, about 7 cP to about 20 cP, about 8 cP to about 10 cP, about 8 cP to about 11 cP, about 8 cP to about 12 cP, about 8 cP to about 13 cP, about 8 cP to about 14 cP, about 8 cP to about 15 cP, about 8 cP to about 16 cP, about 8 cP to about 17 cP, about 8 cP to about 18 cP, about 8 cP to about 19 cP, or about 8 cP to about 20 cP, at a concentration of about 150 mg/ml to about 300 mg/ml, about 150 mg/ml to about 200 mg/ml, or about 150, 155, 160, 165, 170, 175, 180, 185, 190, 195, 200, 205, 210, 215, 220, or 225 mg/ml. In some embodiments, the anti-CD30L antibody or antigen binding fragment is characterized by a viscosity about or less than about 20, 19, 18, 17, 16, 15, 14, 13, 12, 11, 10, 9, 8, 7, 6, or 5 mPa-s at a concentration greater than or about 150 mg/mL. In some embodiments, the anti-CD30L antibody or antigen binding fragment is characterized by a viscosity about or less than about 20, 19, 18, 17, 16, 15, 14, 13, 12, 11, 10, 9, 8, 7, 6, or 5 mPa-s at a concentration greater than or about 160 mg/mL. In some embodiments, the anti-CD30L antibody or antigen binding fragment is characterized by a viscosity about or less than about 20, 19, 18, 17, 16, 15, 14, 13, 12, 11, 10, 9, 8, 7, 6, or 5 mPa-s at a concentration greater than or about 170 mg/mL. In some embodiments, the anti-CD30L antibody or antigen binding fragment is characterized by a viscosity about or less than about 20, 19, 18, 17, 16, 15, 14, 13, 12, 11, 10, 9, 8, 7, 6, or 5 mPa-s at a concentration greater than or about 180 mg/mL. In some embodiments, the anti-CD30L antibody or antigen binding fragment is characterized by a viscosity about or less than about 20, 19, 18, 17, 16, 15, 14, 13, 12, 11, 10, 9, 8, 7, 6, or 5 mPa-s at a concentration greater than or about 190 mg/mL. In some embodiments, the anti-CD30L antibody or antigen binding fragment is characterized by a viscosity about or less than about 20, 19, 18, 17, 16, 15, 14, 13, 12, 11, 10, 9, 8, 7, 6, or 5 mPa-s at a concentration greater than or about 200 mg/mL. In some embodiments, the anti-CD30L antibody or antigen binding fragment is characterized by a viscosity about or less than about 20, 19, 18, 17, 16, 15, 14, 13, 12, 11, 10, 9, 8, 7, 6, or 5 mPa-s at a concentration greater than or about 210 mg/mL. In some embodiments, the anti-CD30L antibody or antigen binding fragment is characterized by a viscosity about or less than about 20, 19, 18, 17, 16, 15, 14, 13, 12, 11, 10, 9, 8, 7, 6, or 5 mPa-s at a concentration greater than or about 220 mg/mL. In some embodiments, the anti-CD30L antibody or antigen binding fragment is characterized by a viscosity about or less than about 20, 19, 18, 17, 16, 15, 14, 13, 12, 11, 10, 9, 8, 7, 6, or 5 mPa-s at a concentration greater than or about 230 mg/mL. In some embodiments, the anti-CD30L antibody or antigen binding fragment is characterized by a viscosity about or less than about 20, 19, 18, 17, 16, 15, 14, 13, 12, 11, 10, 9, 8, 7, 6, or 5 mPa-s at a concentration greater than or about 240 mg/mL. In some embodiments, the anti-CD30L antibody or antigen binding fragment is characterized by a viscosity about or less than about 20, 19, 18, 17, 16, 15, 14, 13, 12, 11, 10, 9, 8, 7, 6, or 5 mPa-s at a concentration greater than or about 250 mg/mL. In some embodiments, the anti-CD30L antibody or antigen binding fragment is characterized by a viscosity about or less than about 20, 19, 18 17, 16, 15, 14, 13, 12, 11, 10, 9, 8, 7, 6, or 5 mPa-s at a concentration of about 150 mg/ml to about 250 mg/ml. In some embodiments, less than about 20 mPa-s includes from about 2 to about 20 mPa-s, from about 2 to about 19 mPa-s, from about 2 to about 18 mPa-s, from about 2 to about 17 mPa-s, from about 2 to about 16 mPa-s, from about 2 to about 15 mPa-s, from about 2 to about 14 mPa-s, from about 2 to about 13 mPa-s, from about 2 to about 12 mPa-s, from about 2 to about 11 mPa-s, from about 2 to about 10 mPa-s, from about 2 to about 9 mPa-s, from about 2 to about 8 mPa-s, from about 2 to about 7 mPa-s, from about 2 to about 6 mPa-s, from about 2 to about 5 mPa-s, from about 3 to about 20 mPa-s, from about 3 to about 19 mPa-s, from about 3 to about 18 mPa-s, from about 3 to about 17 mPa-s, from about 3 to about 16 mPa-s, from about 3 to about 15 mPa-s, from about 3 to about 14 mPa-s, from about 3 to about 13 mPa-s, from about 3 to about 12 mPa-s, from about 3 to about 11 mPa-s, from about 3 to about 10 mPa-s, from about 3 to about 9 mPa-s, from about 3 to about 8 mPa-s, from about 3 to about 7 mPa-s, from about 3 to about 6 mPa-s, from about 3 to about 5 mPa-s, from about 4 to about 20 mPa-s, from about 4 to about 19 mPa-s, from about 4 to about 18 mPa-s, from about 4 to about 17 mPa-s, from about 4 to about 16 mPa-s, from about 4 to about 15 mPa-s, from about 4 to about 14 mPa-s, from about 4 to about 13 mPa-s, from about 4 to about 12 mPa-s, from about 4 to about 11 mPa-s, from about 4 to about 10 mPa-s, from about 4 to about 9 mPa-s, from about 4 to about 8 mPa-s, from about 4 to about 7 mPa-s, from about 4 to about 6 mPa-s, from about 4 to about 5 mPa-s, from about 5 to about 20 mPa-s, from about 5 to about 19 mPa-s, from about 5 to about 18 mPa-s, from about 5 to about 17 mPa-s, from about 5 to about 16 mPa-s, from about 5 to about 15 mPa-s, from about 5 to about 14 mPa-s, from about 5 to about 13 mPa-s, from about 5 to about 12 mPa-s, from about 5 to about 11 mPa-s, from about 5 to about 10 mPa-s, from about 5 to about 9 mPa-s, from about 5 to about 8 mPa-s, from about 5 to about 7 mPa-s, from about 5 to about 6 mPa-s, from about 6 to about 20 mPa-s, from about 6 to about 19 mPa-s, from about 6 to about 18 mPa-s, from about 6 to about 17 mPa-s, from about 6 to about 16 mPa-s, from about 6 to about 15 mPa-s, from about 6 to about 14 mPa-s, from about 6 to about 13 mPa-s, from about 6 to about 12 mPa-s, from about 6 to about 11 mPa-s, from about 6 to about 10 mPa-s, from about 6 to about 9 mPa-s, from about 6 to about 8 mPa-s, or from about 6 to about 7 mPa-s. In some embodiments, greater than about 100, 125, 150, 160, 170, 180, 190, or 200 mg/ml is up to about 250 mg/ml.

4.5.8 Exemplary embodiments of the pharmaceutical composition of anti-CD30L.

The pharmaceutical compositions of anti-CD30L provided herein are formulated to achieve the described concentrations and volumes of the anti-CD30L and a pharmaceutically advantageous profile (including stability, viscosity and safety) with the buffer agents at the provided pH, the excipients, the surfactants, and/or the salts, each as described in details in this Section 4.5 (concentration in Section 4.5.1, buffer agent and pH in Section 4.5.2, excipient in Section 4.5.3, surfactant in Section 4.5.4, salt in Section 4.5.5, volumes in Section 4.5.6, and the pharmaceutically advantageous profiles in Section 4.5.7). As such, the disclosure provides that the pharmaceutical composition of the anti-CD30L provided in Section 4.5 can have any or any combination of (1) concentration as provided in Section 4.5.1, (2) buffer agent and pH as provided in Section 4.5.2, (3) excipient as provided in Section 4.5.3, (4) surfactant as provided in Section 4.5.4, (5) salt as provided in Section 4.5.5, (6) volumes as provided in Section 4.5.6, and/or (7) the pharmaceutically advantageous profiles as provided in Section 4.5.7. Alternatively, the disclosure provides that the pharmaceutical composition of the anti-CD30L provided in Section 4.5 can have any or any combination of (1) concentration as provided in Section 4.5.1, (2) buffer agent and pH as provided in Section 4.5.2, (3) excipient as provided in Section 4.5.3, (4) surfactant as provided in Section 4.5.4, (5) volumes as provided in Section 4.5.6, and/or (6) the pharmaceutically advantageous profiles as provided in Section 4.5.7. Similarly, the disclosure provides that the pharmaceutical composition of the anti-CD30L provided in Section 4.5 can have any or any combination of (1) concentration as provided in Section 4.5.1, (2) buffer agent and pH as provided in Section 4.5.2, (3) excipient as provided in Section 4.5.3, (4) surfactant as provided in Section 4.5.4, and/or (5) the pharmaceutically advantageous profiles as provided in Section 4.5.7. Additionally, the disclosure provides that the pharmaceutical composition of the anti-CD30L provided in Section 4.5 can have any or any combination of (1) concentration as provided in Section 4.5.1, (2) buffer agent and pH as provided in Section 4.5.2, (3) excipient as provided in Section 4.5.3, and/or (4) surfactant as provided in Section 4.5.4.

Accordingly, in certain embodiments, provided herein is a pharmaceutical composition comprising an anti-CD30L antibody or antigen binding fragment thereof provided in Sections 2 and 4.1, for example as provided in Sections 4.5.1, 4.5.2, 4.5.3, 4.5.4, 4.5.5, and/or 4.5.6, wherein the pharmaceutical composition has the stability characteristics and/or other characteristics as provided in Section 4.5.7. In some embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition comprises a buffer provided in Section 4.5.2, wherein the pharmaceutical composition comprises a pharmaceutically acceptable excipient provided in Section 4.5.3, wherein the pharmaceutical composition comprises a surfactant provided in Section 4.5.4, wherein the pharmaceutical composition comprises a salt provided in this Section 4.5.5, and wherein the pharmaceutical composition has the stability characteristics and/or other characteristics as provided in Section 4.5.7. In some embodiments, provided herein is a pharmaceutical composition comprising an anti-CD30L antibody or antigen binding fragment thereof provided in Sections 2 and 4.1, wherein the pharmaceutical composition comprises a salt provided in this Section 4.5.5, and wherein the pharmaceutical composition has the stability characteristics and/or other characteristics as provided in Section 4.5.7. In some embodiments, provided herein is a pharmaceutical composition comprising an anti-CD30L antibody or antigen binding fragment thereof provided in Sections 2 and 4.1, wherein the pharmaceutical composition comprises a surfactant provided in Section 4.5.4, wherein the pharmaceutical composition comprises a salt provided in this Section 4.5.5, and wherein the pharmaceutical composition has the stability characteristics and/or other characteristics as provided in Section 4.5.7. In certain embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition comprises a salt provided in this Section 4.5.5, and wherein the pharmaceutical composition has the stability characteristics and/or other characteristics as provided in Section 4.5.7. In certain embodiments, provided herein is a pharmaceutical composition comprising an anti-CD30L antibody or antigen binding fragment thereof provided in Sections 2 and 4.1, wherein the pharmaceutical composition comprises a buffer provided in Section 4.5.2, wherein the pharmaceutical composition comprises a salt provided in this Section 4.5.5, and wherein the pharmaceutical composition has the stability characteristics and/or other characteristics as provided in Section 4.5.7. In some embodiments, provided herein is a pharmaceutical composition comprising an anti-CD30L antibody or antigen binding fragment thereof provided in Sections 2 and 4.1, wherein the pharmaceutical composition comprises a pharmaceutically acceptable excipient provided in Section 4.5.3, wherein the pharmaceutical composition comprises a salt provided in this Section 4.5.5, and wherein the pharmaceutical composition has the stability characteristics and/or other characteristics as provided in Section 4.5.7. In certain embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition comprises a surfactant provided in Section 4.5.4, wherein the pharmaceutical composition comprises a salt provided in this Section 4.5.5, and wherein the pharmaceutical composition has the stability characteristics and/or other characteristics as provided in Section 4.5.7. In certain embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition comprises a buffer provided in Section 4.5.2, wherein the pharmaceutical composition comprises a salt provided in this Section 4.5.5, and wherein the pharmaceutical composition has the stability characteristics and/or other characteristics as provided in Section 4.5.7. In certain embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition comprises a pharmaceutically acceptable excipient provided in Section 4.5.3, wherein the pharmaceutical composition comprises a salt provided in this Section 4.5.5, and wherein the pharmaceutical composition has the stability characteristics and/or other characteristics as provided in Section 4.5.7. In certain embodiments, provided herein is a pharmaceutical composition comprising an anti-CD30L antibody or antigen binding fragment thereof provided in Sections 2 and 4.1, wherein the pharmaceutical composition comprises a buffer provided in Section 4.5.2, wherein the pharmaceutical composition comprises a pharmaceutically acceptable excipient provided in Section 4.5.3, wherein the pharmaceutical composition comprises a salt provided in this Section 4.5.5, and wherein the pharmaceutical composition has the stability characteristics and/or other characteristics as provided in Section 4.5.7. In certain embodiments, provided herein is a pharmaceutical composition comprising an anti-CD30L antibody or antigen binding fragment thereof provided in Sections 2 and 4.1, wherein the pharmaceutical composition comprises a buffer provided in Section 4.5.2, wherein the pharmaceutical composition comprises a surfactant provided in Section 4.5.4, wherein the pharmaceutical composition comprises a salt provided in this Section 4.5.5, and wherein the pharmaceutical composition has the stability characteristics and/or other characteristics as provided in Section 4.5.7. In some embodiments, provided herein is a pharmaceutical composition comprising an anti-CD30L antibody or antigen binding fragment thereof provided in Sections 2 and 4.1, wherein the pharmaceutical composition comprises a pharmaceutically acceptable excipient provided in Section 4.5.3, wherein the pharmaceutical composition comprises a surfactant provided in Section 4.5.4, wherein the pharmaceutical composition comprises a salt provided in this Section 4.5.5, and wherein the pharmaceutical composition has the stability characteristics and/or other characteristics as provided in Section 4.5.7. In some embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition comprises a buffer provided in Section 4.5.2, wherein the pharmaceutical composition comprises a pharmaceutically acceptable excipient provided in Section 4.5.3, wherein the pharmaceutical composition comprises a salt provided in this Section 4.5.5, and wherein the pharmaceutical composition has the stability characteristics and/or other characteristics as provided in Section 4.5.7. In some embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition comprises a buffer provided in Section 4.5.2, wherein the pharmaceutical composition comprises a surfactant provided in Section 4.5.4, wherein the pharmaceutical composition comprises a salt provided in this Section 4.5.5, and wherein the pharmaceutical composition has the stability characteristics and/or other characteristics as provided in Section 4.5.7. In some embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition comprises a pharmaceutically acceptable excipient provided in Section 4.5.3, wherein the pharmaceutical composition comprises a surfactant provided in Section 4.5.4, wherein the pharmaceutical composition comprises a salt provided in this Section 4.5.5, and wherein the pharmaceutical composition has the stability characteristics and/or other characteristics as provided in Section 4.5.7. In some embodiments, provided herein is a pharmaceutical composition comprising an anti-CD30L antibody or antigen binding fragment thereof provided in Sections 2 and 4.1, wherein the pharmaceutical composition comprises a buffer provided in Section 4.5.2, wherein the pharmaceutical composition comprises a pharmaceutically acceptable excipient provided in Section 4.5.3, wherein the pharmaceutical composition comprises a surfactant provided in Section 4.5.4, wherein the pharmaceutical composition comprises a salt provided in this Section 4.5.5, and wherein the pharmaceutical composition has the stability characteristics and/or other characteristics as provided in Section 4.5.7. In some embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition comprises a buffer provided in Section 4.5.2, wherein the pharmaceutical composition comprises a pharmaceutically acceptable excipient provided in Section 4.5.3, wherein the pharmaceutical composition comprises a surfactant provided in this Section 4.5.4, and wherein the pharmaceutical composition has the stability characteristics and/or other characteristics as provided in Section 4.5.7. In some embodiments, provided herein is a pharmaceutical composition comprising an anti-CD30L antibody or antigen binding fragment thereof provided in Sections 2 and 4.1, wherein the pharmaceutical composition comprises a surfactant provided in this Section 4.5.4, and wherein the pharmaceutical composition has the stability characteristics and/or other characteristics as provided in Section 4.5.7. In certain embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition comprises a surfactant provided in this Section 4.5.4, and wherein the pharmaceutical composition has the stability characteristics and/or other characteristics as provided in Section 4.5.7. In certain embodiments, provided herein is a pharmaceutical composition comprising an anti-CD30L antibody or antigen binding fragment thereof provided in Sections 2 and 4.1, wherein the pharmaceutical composition comprises a buffer provided in Section 4.5.2, wherein the pharmaceutical composition comprises a surfactant provided in this Section 4.5.4, and wherein the pharmaceutical composition has the stability characteristics and/or other characteristics as provided in Section 4.5.7. In some embodiments, provided herein is a pharmaceutical composition comprising an anti-CD30L antibody or antigen binding fragment thereof provided in Sections 2 and 4.1, wherein the pharmaceutical composition comprises a pharmaceutically acceptable excipient provided in Section 4.5.3, wherein the pharmaceutical composition comprises a surfactant provided in this Section 4.5.4, and wherein the pharmaceutical composition has the stability characteristics and/or other characteristics as provided in Section 4.5.7. In some embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition comprises a buffer provided in Section 4.5.2, wherein the pharmaceutical composition comprises a surfactant provided in this Section 4.5.4, and wherein the pharmaceutical composition has the stability characteristics and/or other characteristics as provided in Section 4.5.7. In some embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition comprises a pharmaceutically acceptable excipient provided in Section 4.5.3, wherein the pharmaceutical composition comprises a surfactant provided in this Section 4.5.4, and wherein the pharmaceutical composition has the stability characteristics and/or other characteristics as provided in Section 4.5.7. In some embodiments, provided herein is a pharmaceutical composition comprising an anti-CD30L antibody or antigen binding fragment thereof provided in Sections 2 and 4.1, wherein the pharmaceutical composition comprises a buffer provided in Section 4.5.2, wherein the pharmaceutical composition comprises a pharmaceutically acceptable excipient provided in Section 4.5.3, wherein the pharmaceutical composition comprises a surfactant provided in this Section 4.5.4, and wherein the pharmaceutical composition has the stability characteristics and/or other characteristics as provided in Section 4.5.7. In some embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition comprises a buffer provided in Section 4.5.2, wherein the pharmaceutical composition comprises a pharmaceutically acceptable excipient provided in this Section 4.5.3, and wherein the pharmaceutical composition has the stability characteristics and/or other characteristics as provided in Section 4.5.7. In some embodiments, provided herein is a pharmaceutical composition comprising an anti-CD30L antibody or antigen binding fragment thereof provided in Sections 2 and 4.1, wherein the pharmaceutical composition comprises a pharmaceutically acceptable excipient provided in this Section 4.5.3, and wherein the pharmaceutical composition has the stability characteristics and/or other characteristics as provided in Section 4.5.7. In certain embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition comprises a pharmaceutically acceptable excipient provided in this Section 4.5.3, and wherein the pharmaceutical composition has the stability characteristics and/or other characteristics as provided in Section 4.5.7. in certain embodiments, provided herein is a pharmaceutical composition comprising an anti-CD30L antibody or antigen binding fragment thereof provided in Sections 2 and 4.1, wherein the pharmaceutical composition comprises a buffer provided in Section 4.5.2, wherein the pharmaceutical composition comprises a pharmaceutically acceptable excipient provided in this Section 4.5.3, and wherein the pharmaceutical composition has the stability characteristics and/or other characteristics as provided in Section 4.5.7. In some embodiments, provided herein is a pharmaceutical composition comprising an anti-CD30L antibody or antigen binding fragment thereof provided in Sections 2 and 4.1, at a concentration provided in Section 4.5.1, wherein the pharmaceutical composition further comprises a buffer provided in this Section 4.5.2, and wherein the pharmaceutical composition has the stability characteristics and/or other characteristics as provided in Section 4.5.7. In certain embodiments, provided herein is a pharmaceutical composition comprising an anti-CD30L antibody or antigen binding fragment thereof provided in Sections 2 and 4.1, wherein the pharmaceutical composition further comprises a buffer provided in this Section 4.5.2, and wherein the pharmaceutical composition has the stability characteristics and/or other characteristics as provided in Section 4.5.7. In some embodiments, provided herein is a pharmaceutical composition comprising an anti-CD30L antibody or antigen binding fragment thereof provided in Sections 2 and 4.1, at a concentration provided in this Section 4.5.1, and wherein the pharmaceutical composition has the stability characteristics and/or other characteristics as provided in Section 4.5.7. For each of the embodiment provided in this paragraph, the additional embodiment is also provided, wherein the pharmaceutical composition has a volume selected from the volumes provided in Section 4.5.6.

Specifically and as an example of the forgoing paragraphs in this Section 4.5, in some embodiments, the pharmaceutical composition comprises (i) an anti-CD30L antibody or antigen-binding fragment provided herein, e.g. in Section 4.1, (ii) a surfactant, (iii) a histidine buffer, (iv) arginine HCl as the excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL. In some embodiments, the pharmaceutical composition comprises (i) an anti-CD30L antibody or antigen-binding fragment provided herein, e.g. in Section 4.1, (ii) a surfactant, (iii) a histidine buffer, (iv) arginine HCl as the excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL, and wherein the surfactant is PS20 at a concentration of about 0.01% to 0.03% (w/v) (e.g. about 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, 0.03% (w/v)). In some embodiments, the pharmaceutical composition comprises (i) an anti-CD30L antibody or antigen-binding fragment provided herein, e.g. in Section 4.1, (ii) a surfactant, (iii) a histidine buffer, (iv) arginine HCl as the excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL, wherein the surfactant is PS20 at a concentration of about 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, 0.03% (w/v), and wherein the histidine buffer is at a concentration of 10 mM to 30 mM (e.g. about 10, 11, 12, 13, 14, 15, 16, 17, 18, 19, 20, 21, 22, 23, 24, 25, 26, 27, 28, 29, or 30 mM) for pH of 5.0 to 6.0 (e.g. pH of about 5.1, 5.2, 5.3, 5.4, 5.5, 5.6, 5.7, 5.8, 5.9, or 6.0). In some embodiments, the pharmaceutical composition comprises (i) an anti-CD30L antibody or antigen-binding fragment provided herein, e.g. in Section 4.1, (ii) a surfactant, (iii) a histidine buffer, (iv) arginine HCl as the excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL, wherein the surfactant is PS20 at a concentration of about 0.01% to 0.03% (w/v) (e.g. about 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, or 0.03% (w/v)), wherein the histidine buffer is at a concentration of 10 mM to 30 mM (e.g. about 10, 11, 12, 13, 14, 15, 16, 17, 18, 19, 20, 21, 22, 23, 24, 25, 26, 27, 28, 29, or 30 mM) for pH of 5.0 to 6.0 (e.g. pH of about 5.1, 5.2, 5.3, 5.4, 5.5, 5.6, 5.7, 5.8, 5.9, or 6.0), and wherein the arginine HCl is at a concentration of 50 mM to 150 mM (e.g. about 50 mM, about 60 mM, about 70 mM, about 80 mM, about 90 mM, about 100 mM, about 110 mM, about 120 mM, about 130 mM, about 140 mM, or about 150 mM). In some embodiments, the pharmaceutical composition comprises (i) an anti-CD30L antibody or antigen-binding fragment provided herein, e.g. in Section 4.1, (ii) a surfactant, (iii) a histidine buffer, (iv) arginine HCl as the excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL, wherein the surfactant is PS20 at a concentration of about 0.01% to 0.03% (w/v) (e.g. about 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, 0.03% (w/v)), and wherein the histidine buffer is at a concentration of 10 mM to 50 mM (e.g. about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mM) for pH of 5.0 to 6.0 (e.g. pH of about 5.1, 5.2, 5.3, 5.4, 5.5, 5.6, 5.7, 5.8, 5.9, or 6.0). In some embodiments, the pharmaceutical composition comprises (i) an anti-CD30L antibody or antigen-binding fragment provided herein, e.g. in Section 4.1, (ii) a surfactant, (iii) a histidine buffer, (iv) arginine HCl as the excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL, wherein the surfactant is PS20 at a concentration of about 0.01% to 0.03% (w/v) (e.g. about 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, or 0.03% (w/v)), wherein the histidine buffer is at a concentration of 10 mM to 50 mM (e.g. about 10, 11, 12, 13, 14, 15, 16, 17, 18, 19, 20, 21, 22, 23, 24, 25, 26, 27, 28, 29, or 30 mM) for pH of 5.0 to 6.0 (e.g. pH of about 5.1, 5.2, 5.3, 5.4, 5.5, 5.6, 5.7, 5.8, 5.9, or 6.0), and wherein the arginine HCl is at a concentration of 50 mM to 250 mM (e.g. about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, or about 250 mM).

As another example of the pharmaceutical composition provided in this Section 4.5, in some embodiments, the pharmaceutical composition comprises (i) an anti-CD30L antibody or antigen-binding fragment provided herein, e.g. in Section 4.1, (ii) a surfactant, (iii) an acetate buffer, (iv) arginine HCl as the excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL. In some embodiments, the pharmaceutical composition comprises (i) an anti-CD30L antibody or antigen-binding fragment provided herein, e.g. in Section 4.1, (ii) a surfactant, (iii) an acetate buffer, (iv) arginine HCl as the excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL, and wherein the surfactant is PS20 at a concentration of about 0.01% to 0.03% (w/v) (e.g. about 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, 0.03% (w/v)). In some embodiments, the pharmaceutical composition comprises (i) an anti-CD30L antibody or antigen-binding fragment provided herein, e.g. in Section 4.1, (ii) a surfactant, (iii) an acetate buffer, (iv) arginine HCl as the excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL, wherein the surfactant is PS20 at a concentration of about 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, 0.03% (w/v), and wherein the acetate buffer is at a concentration of 10 mM to 30 mM (e.g. about 10, 11, 12, 13, 14, 15, 16, 17, 18, 19, 20, 21, 22, 23, 24, 25, 26, 27, 28, 29, or 30 mM) for pH of 5.0 to 6.0 (e.g. pH of about 5.1, 5.2, 5.3, 5.4, 5.5, 5.6, 5.7, 5.8, 5.9, or 6.0). In some embodiments, the pharmaceutical composition comprises (i) an anti-CD30L antibody or antigen-binding fragment provided herein, e.g. in Section 4.1, (ii) a surfactant, (iii) an acetate buffer, (iv) arginine HCl as the excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL, wherein the surfactant is PS20 at a concentration of about 0.01% to 0.03% (w/v) (e.g. about 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, or 0.03% (w/v)), wherein the acetate buffer is at a concentration of 10 mM to 30 mM (e.g. about 10, 11, 12, 13, 14, 15, 16, 17, 18, 19, 20, 21, 22, 23, 24, 25, 26, 27, 28, 29, or 30 mM) for pH of 5.0 to 6.0 (e.g. pH of about 5.1, 5.2, 5.3, 5.4, 5.5, 5.6, 5.7, 5.8, 5.9, or 6.0), and wherein the arginine HCl is at a concentration of 50 mM to 150 mM (e.g. about 50 mM, about 60 mM, about 70 mM, about 80 mM, about 90 mM, about 100 mM, about 110 mM, about 120 mM, about 130 mM, about 140 mM, or about 150 mM). In some embodiments, the pharmaceutical composition comprises (i) an anti-CD30L antibody or antigen-binding fragment provided herein, e.g. in Section 4.1, (ii) a surfactant, (iii) an acetate buffer, (iv) arginine HCl as the excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL, wherein the surfactant is PS20 at a concentration of about 0.01% to 0.03% (w/v) (e.g. about 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, 0.03% (w/v)), and wherein the acetate buffer is at a concentration of 10 mM to 50 mM (e.g. about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mM) for pH of 5.0 to 6.0 (e.g. pH of about 5.1, 5.2, 5.3, 5.4, 5.5, 5.6, 5.7, 5.8, 5.9, or 6.0). In some embodiments, the pharmaceutical composition comprises (i) an anti-CD30L antibody or antigen-binding fragment provided herein, e.g. in Section 4.1, (ii) a surfactant, (iii) an acetate buffer, (iv) arginine HCl as the excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL, wherein the surfactant is PS20 at a concentration of about 0.01% to 0.03% (w/v) (e.g. about 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, or 0.03% (w/v)), wherein the acetate buffer is at a concentration of 10 mM to 50 mM (e.g. about 10, 11, 12, 13, 14, 15, 16, 17, 18, 19, 20, 21, 22, 23, 24, 25, 26, 27, 28, 29, or 30 mM) for pH of 5.0 to 6.0 (e.g. pH of about 5.1, 5.2, 5.3, 5.4, 5.5, 5.6, 5.7, 5.8, 5.9, or 6.0), and wherein the arginine HCl is at a concentration of 50 mM to 250 mM (e.g. about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, or about 250 mM).

As a further example of the pharmaceutical composition provided in this Section 4.5, in some embodiments, the pharmaceutical composition comprises (i) an anti-CD30L antibody or antigen-binding fragment provided herein, e.g. in Section 4.1, (ii) a surfactant, (iii) a succinate buffer, (iv) arginine HCl as the excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL. In some embodiments, the pharmaceutical composition comprises (i) an anti-CD30L antibody or antigen-binding fragment provided herein, e.g. in Section 4.1, (ii) a surfactant, (iii) a succinate buffer, (iv) arginine HCl as the excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL, and wherein the surfactant is PS20 at a concentration of about 0.01% to 0.03% (w/v) (e.g. about 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, 0.03% (w/v)). In some embodiments, the pharmaceutical composition comprises (i) an anti-CD30L antibody or antigen-binding fragment provided herein, e.g. in Section 4.1, (ii) a surfactant, (iii) a succinate buffer, (iv) arginine HCl as the excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL, wherein the surfactant is PS20 at a concentration of about 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, 0.03% (w/v), and wherein the succinate buffer is at a concentration of 10 mM to 30 mM (e.g. about 10, 11, 12, 13, 14, 15, 16, 17, 18, 19, 20, 21, 22, 23, 24, 25, 26, 27, 28, 29, or 30 mM) for pH of 5.0 to 6.0 (e.g. pH of about 5.1, 5.2, 5.3, 5.4, 5.5, 5.6, 5.7, 5.8, 5.9, or 6.0). In some embodiments, the pharmaceutical composition comprises (i) an anti-CD30L antibody or antigen-binding fragment provided herein, e.g. in Section 4.1, (ii) a surfactant, (iii) a succinate buffer, (iv) arginine HCl as the excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL, wherein the surfactant is PS20 at a concentration of about 0.01% to 0.03% (w/v) (e.g. about 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, or 0.03% (w/v)), wherein the succinate buffer is at a concentration of 10 mM to 30 mM (e.g. about 10, 11, 12, 13, 14, 15, 16, 17, 18, 19, 20, 21, 22, 23, 24, 25, 26, 27, 28, 29, or 30 mM) for pH of 5.0 to 6.0 (e.g. pH of about 5.1, 5.2, 5.3, 5.4, 5.5, 5.6, 5.7, 5.8, 5.9, or 6.0), and wherein the arginine HCl is at a concentration of 50 mM to 150 mM (e.g. about 50 mM, about 60 mM, about 70 mM, about 80 mM, about 90 mM, about 100 mM, about 110 mM, about 120 mM, about 130 mM, about 140 mM, or about 150 mM). In some embodiments, the pharmaceutical composition comprises (i) an anti-CD30L antibody or antigen-binding fragment provided herein, e.g. in Section 4.1, (ii) a surfactant, (iii) a succinate buffer, (iv) arginine HCl as the excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL, wherein the surfactant is PS20 at a concentration of about 0.01% to 0.03% (w/v) (e.g. about 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, 0.03% (w/v)), and wherein the succinate buffer is at a concentration of 10 mM to 50 mM (e.g. about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mM) for pH of 5.0 to 6.0 (e.g. pH of about 5.1, 5.2, 5.3, 5.4, 5.5, 5.6, 5.7, 5.8, 5.9, or 6.0). In some embodiments, the pharmaceutical composition comprises (i) an anti-CD30L antibody or antigen-binding fragment provided herein, e.g. in Section 4.1, (ii) a surfactant, (iii) a succinate buffer, (iv) arginine HCl as the excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL, wherein the surfactant is PS20 at a concentration of about 0.01% to 0.03% (w/v) (e.g. about 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, or 0.03% (w/v)), wherein the succinate buffer is at a concentration of 10 mM to 50 mM (e.g. about 10, 11, 12, 13, 14, 15, 16, 17, 18, 19, 20, 21, 22, 23, 24, 25, 26, 27, 28, 29, or 30 mM) for pH of 5.0 to 6.0 (e.g. pH of about 5.1, 5.2, 5.3, 5.4, 5.5, 5.6, 5.7, 5.8, 5.9, or 6.0), and wherein the arginine HCl is at a concentration of 50 mM to 250 mM (e.g. about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, or about 250 mM).

As yet another example of the pharmaceutical composition provided in this Section 4.5, in some embodiments, the pharmaceutical composition comprises (i) an anti-CD30L antibody or antigen-binding fragment provided herein, e.g. in Section 4.1, (ii) a surfactant, (iii) a histidine buffer, (iv) L-proline as the excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL. In some embodiments, the pharmaceutical composition comprises (i) an anti-CD30L antibody or antigen-binding fragment provided herein, e.g. in Section 4.1, (ii) a surfactant, (iii) a histidine buffer, (iv) L-proline as the excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL, and wherein the surfactant is PS20 at a concentration of about 0.01% to 0.03% (w/v) (e.g. about 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, 0.03% (w/v)). In some embodiments, the pharmaceutical composition comprises (i) an anti-CD30L antibody or antigen-binding fragment provided herein, e.g. in Section 4.1, (ii) a surfactant, (iii) a histidine buffer, (iv) L-proline as the excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL, wherein the surfactant is PS20 at a concentration of about 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, 0.03% (w/v), and wherein the histidine buffer is at a concentration of 10 mM to 30 mM (e.g. about 10, 11, 12, 13, 14, 15, 16, 17, 18, 19, 20, 21, 22, 23, 24, 25, 26, 27, 28, 29, or 30 mM) for pH of 5.0 to 6.0 (e.g. pH of about 5.1, 5.2, 5.3, 5.4, 5.5, 5.6, 5.7, 5.8, 5.9, or 6.0). In some embodiments, the pharmaceutical composition comprises (i) an anti-CD30L antibody or antigen-binding fragment provided herein, e.g. in Section 4.1, (ii) a surfactant, (iii) a histidine buffer, (iv) L-proline as the excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL, wherein the surfactant is PS20 at a concentration of about 0.01% to 0.03% (w/v) (e.g. about 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, or 0.03% (w/v)), wherein the histidine buffer is at a concentration of 10 mM to 30 mM (e.g. about 10, 11, 12, 13, 14, 15, 16, 17, 18, 19, 20, 21, 22, 23, 24, 25, 26, 27, 28, 29, or 30 mM) for pH of 5.0 to 6.0 (e.g. pH of about 5.1, 5.2, 5.3, 5.4, 5.5, 5.6, 5.7, 5.8, 5.9, or 6.0), and wherein the L-proline is at a concentration of 150 mM to 300 mM (e.g. about 150 mM, about 160 mM, about 170 mM, about 180 mM, about 190 mM, about 200 mM, about 210 mM, about 220 mM, about 230 mM, about 240 mM, about 245 mM, about 250 mM, about 260 mM, about 270 mM, about 280 mM, about 290 mM, or about 300 mM). In some embodiments, the pharmaceutical composition comprises (i) an anti-CD30L antibody or antigen-binding fragment provided herein, e.g. in Section 4.1, (ii) a surfactant, (iii) a histidine buffer, (iv) L-proline as the excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL, wherein the surfactant is PS20 at a concentration of about 0.01% to 0.03% (w/v) (e.g. about 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, 0.03% (w/v)), and wherein the histidine buffer is at a concentration of 10 mM to 50 mM (e.g. about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mM) for pH of 5.0 to 6.0 (e.g. pH of about 5.1, 5.2, 5.3, 5.4, 5.5, 5.6, 5.7, 5.8, 5.9, or 6.0). In some embodiments, the pharmaceutical composition comprises (i) an anti-CD30L antibody or antigen-binding fragment provided herein, e.g. in Section 4.1, (ii) a surfactant, (iii) a histidine buffer, (iv) L-proline as the excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL, wherein the surfactant is PS20 at a concentration of about 0.01% to 0.03% (w/v) (e.g. about 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, or 0.03% (w/v)), wherein the histidine buffer is at a concentration of 10 mM to 50 mM (e.g. about 10, 11, 12, 13, 14, 15, 16, 17, 18, 19, 20, 21, 22, 23, 24, 25, 26, 27, 28, 29, or 30 mM) for pH of 5.0 to 6.0 (e.g. pH of about 5.1, 5.2, 5.3, 5.4, 5.5, 5.6, 5.7, 5.8, 5.9, or 6.0), and wherein the L-proline is at a concentration of 100 mM to 350 mM (e.g. about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 245 mM, about 250 mM, about 260 mM, about 270 mM, about 280 mM, about 290 mM, about 300 mM, about 310 mM, about 320 mM, about 330 mM, about 340 mM, or about 350 mM).

As another example of the pharmaceutical composition provided in this Section 4.5, in some embodiments, the pharmaceutical composition comprises (i) an anti-CD30L antibody or antigen-binding fragment provided herein, e.g. in Section 4.1, (ii) a surfactant, (iii) a buffer agent, (iv) an excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL. In some embodiments, the pharmaceutical composition comprises (i) an anti-CD30L antibody or antigen-binding fragment provided herein, e.g. in Section 4.1, (ii) a surfactant, (iii) a buffer agent, (iv) an excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL, and wherein the surfactant is PS20 at a concentration of about 0.01% to 0.03% (w/v) (e.g. about 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, 0.03% (w/v)). In some embodiments, the pharmaceutical composition comprises (i) an anti-CD30L antibody or antigen-binding fragment provided herein, e.g. in Section 4.1, (ii) a surfactant, (iii) a buffer agent, (iv) an excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL, wherein the surfactant is PS20 at a concentration of about 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, 0.03% (w/v), and wherein the buffer agent is histidine, succinate, or acetate at a concentration of 10 mM to 30 mM (e.g. about 10, 11, 12, 13, 14, 15, 16, 17, 18, 19, 20, 21, 22, 23, 24, 25, 26, 27, 28, 29, or 30 mM) for pH of 5.0 to 6.0 (e.g. pH of about 5.1, 5.2, 5.3, 5.4, 5.5, 5.6, 5.7, 5.8, 5.9, or 6.0). In some embodiments, the pharmaceutical composition comprises (i) an anti-CD30L antibody or antigen-binding fragment provided herein, e.g. in Section 4.1, (ii) a surfactant, (iii) a buffer agent, (iv) an excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL, wherein the surfactant is PS20 at a concentration of about 0.01% to 0.03% (w/v) (e.g. about 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, or 0.03% (w/v)), wherein the buffer agent is histidine, succinate, or acetate at a concentration of 10 mM to 30 mM (e.g. about 10, 11, 12, 13, 14, 15, 16, 17, 18, 19, 20, 21, 22, 23, 24, 25, 26, 27, 28, 29, or 30 mM) for pH of 5.0 to 6.0 (e.g. pH of about 5.1, 5.2, 5.3, 5.4, 5.5, 5.6, 5.7, 5.8, 5.9, or 6.0), and wherein the excipient is arginine HCl or L-proline at a concentration of 50 mM to 300 mM (e.g. about 50 mM, about 60 mM, about 70 mM, about 80 mM, about 90 mM, about 100 mM, about 110 mM, about 120 mM, about 130 mM, about 140 mM, or about 150 mM, about 160 mM, about 170 mM, about 180 mM, about 190 mM, about 200 mM, about 210 mM, about 220 mM, about 230 mM, about 240 mM, about 245 mM, about 250 mM, about 260 mM, about 270 mM, about 280 mM, about 290 mM, or about 300 mM). In some embodiments, the pharmaceutical composition comprises (i) an anti-CD30L antibody or antigen-binding fragment provided herein, e.g. in Section 4.1, (ii) a surfactant, (iii) a buffer agent, (iv) an excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL, wherein the surfactant is PS20 at a concentration of about 0.01% to 0.03% (w/v) (e.g. about 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, 0.03% (w/v)), and wherein the buffer agent is histidine, succinate, or acetate at a concentration of 10 mM to 50 mM (e.g. about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mM) for pH of 5.0 to 6.0 (e.g. pH of about 5.1, 5.2, 5.3, 5.4, 5.5, 5.6, 5.7, 5.8, 5.9, or 6.0). In some embodiments, the pharmaceutical composition comprises (i) an anti-CD30L antibody or antigen-binding fragment provided herein, e.g. in Section 4.1, (ii) a surfactant, (iii) a buffer agent, (iv) an excipient, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL, wherein the surfactant is PS20 at a concentration of about 0.01% to 0.03% (w/v) (e.g. about 0.01%, 0.011%, 0.012%, 0.013%, 0.014%, 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, or 0.03% (w/v)), wherein the buffer agent is histidine, succinate, or acetate at a concentration of 10 mM to 50 mM (e.g. about 10, 11, 12, 13, 14, 15, 16, 17, 18, 19, 20, 21, 22, 23, 24, 25, 26, 27, 28, 29, or 30 mM) for pH of 5.0 to 6.0 (e.g. pH of about 5.1, 5.2, 5.3, 5.4, 5.5, 5.6, 5.7, 5.8, 5.9, or 6.0), and wherein the excipient is arginine HCl or L-proline at a concentration of 50 mM to 350 mM (e.g. about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, or about 350 mM).

In some embodiments of the pharmaceutical composition comprising an anti-CD30L antibody or antigen binding fragment thereof provided in Sections 2 and 4.1, for example as provided in Sections 4.5.1, 4.5.2, 4.5.3, 4.5.4, 4.5.5, 4.5.6, 4.5.7, and/or 4.5.8, wherein the pharmaceutical composition further comprises an anti-oxidant. In some embodiments of the pharmaceutical composition comprising an anti-CD30L antibody or antigen binding fragment thereof provided in Sections 2 and 4.1, for example as provided in Sections 4.5.1, 4.5.2, 4.5.3, 4.5.4, 4.5.5, 4.5.6, 4.5.7, and/or 4.5.8, wherein the pharmaceutical composition further comprises an anti-oxidant, and wherein the anti-oxidant is L-Methionine at a concentration of about 1 mM to about 50 mM (e.g. about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mM). In some embodiments of the pharmaceutical composition comprising an anti-CD30L antibody or antigen binding fragment thereof provided in Sections 2 and 4.1, for example as provided in Sections 4.5.1, 4.5.2, 4.5.3, 4.5.4, 4.5.5, 4.5.6, 4.5.7, and/or 4.5.8, wherein the pharmaceutical composition further comprises an anti-oxidant, and wherein the anti-oxidant is L-Methionine at a concentration of about 10 mM.

In some embodiments of the pharmaceutical composition comprising an anti-CD30L antibody or antigen binding fragment thereof provided in Sections 2 and 4.1, for example as provided in Sections 4.5.1, 4.5.2, 4.5.3, 4.5.4, 4.5.5, 4.5.6, 4.5.7, and/or 4.5.8, wherein the pharmaceutical composition does not comprise an anti-oxidant. In certain embodiments of the pharmaceutical composition comprising an anti-CD30L antibody or antigen binding fragment thereof provided in Sections 2 and 4.1, for example as provided in this Section 4.5 (including but not limited to the foregoing paragraphs of this Section 4.5 such as the foregoing paragraphs of this subsection 4.5.8,), wherein the pharmaceutical composition does not comprise an anti-oxidant. The disclosure further provides that the omission of the anti-oxidant provides an advantage for the pharmaceutical compositions because of the reduce complexity in manufacturing and human safety evaluation. As is clear from Section 5, the pharmaceutical composition provided herein can maintain the advantageous and desirable properties during storage (such as those provided in Sections 4.5.7 and 5) despite the absence of an anti-oxidant.

In one embodiment, provided herein is a pharmaceutical composition comprising an anti-CD30L antibody or antigen binding fragment provided in Section 4.1 at a concentration of 200 mg/mL, 20 mM histidine buffer, 100 mM arginine monohydrochloride, and 0.02% (w/v) PS20, at pH 5.5. In another embodiment, provided herein is a pharmaceutical composition comprising an anti-CD30L antibody or antigen binding fragment at a concentration of 200 mg/mL, 20 mM histidine buffer, 100 mM arginine monohydrochloride, and 0.02% (w/v) PS20, at pH 5.5. In yet another embodiment, provided herein is a pharmaceutical composition comprising an anti-CD30L antibody or antigen binding fragment, 20 mM histidine buffer, 100 mM arginine monohydrochloride, and 0.02% (w/v) PS20, at pH 5.5, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL. In a further embodiment, provided herein is a pharmaceutical composition comprising an anti-CD30L antibody or antigen binding fragment selected from those provided in Section 4.1, 20 mM histidine buffer, 100 mM arginine monohydrochloride, and 0.02% (w/v) PS20, at pH 5.5, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL.

In one embodiment, provided herein is a pharmaceutical composition comprising an anti-CD30L antibody or antigen binding fragment at a concentration of 200 mg/mL, 20 mM histidine buffer, 100 mM arginine monohydrochloride, and 0.02% (w/v) PS20, at pH 5.5, wherein the anti-CD30L comprises a CDR-H1 comprising the amino acid sequence set forth in any one of SEQ ID NOs: 110-119, 629, 636, and 712-723, a CDR-H2 comprising the amino acid sequence set forth in any one of SEQ ID NOs: 150-159, 643, 650, and 724-735, a CDR-H3 comprising the amino acid sequence set forth in any one of SEQ ID NOs: 190-199, 657, 664, and 736-743, a CDR-L1 comprising the amino acid sequence set forth in any one of SEQ ID NOs: 310-319, 671, 678, and 744-751, a CDR-L2 comprising the amino acid sequence set forth in any one of SEQ ID NOs: 350-359, 685, 692, and 752-759, and a CDR-L3 comprising the amino acid sequence set forth in any one of SEQ ID NOs: 390-399, 699, 706, and 760-765. In yet another embodiment, provided herein is a pharmaceutical composition comprising an anti-CD30L antibody or antigen binding fragment, 20 mM histidine buffer, 100 mM arginine monohydrochloride, and 0.02% (w/v) PS20, at pH 5.5, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL, and wherein the anti-CD30L comprises a CDR-H1 comprising the amino acid sequence set forth in any one of SEQ ID NOs: 110-119, 629, 636, and 712-723, a CDR-H2 comprising the amino acid sequence set forth in any one of SEQ ID NOs: 150-159, 643, 650, and 724-735, a CDR-H3 comprising the amino acid sequence set forth in any one of SEQ ID NOs: 190-199, 657, 664, and 736-743, a CDR-L1 comprising the amino acid sequence set forth in any one of SEQ ID NOs: 310-319, 671, 678, and 744-751, a CDR-L2 comprising the amino acid sequence set forth in any one of SEQ ID NOs: 350-359, 685, 692, and 752-759, and a CDR-L3 comprising the amino acid sequence set forth in any one of SEQ ID NOs: 390-399, 699, 706, and 760-765.

In one embodiment, provided herein is a pharmaceutical composition comprising an anti-CD30L antibody or antigen binding fragment provided in Section 4.1 at a concentration of 200 mg/mL, 20 mM histidine buffer, 100 mM arginine monohydrochloride, 10 mM L-Methionine, and 0.02% (w/v) PS20, at pH 5.5. In another embodiment, provided herein is a pharmaceutical composition comprising an anti-CD30L antibody or antigen binding fragment at a concentration of 200 mg/mL, 20 mM histidine buffer, 100 mM arginine monohydrochloride, 10 mM L-Methionine, and 0.02% (w/v) PS20, at pH 5.5. In yet another embodiment, provided herein is a pharmaceutical composition comprising an anti-CD30L antibody or antigen binding fragment, 20 mM histidine buffer, 100 mM arginine monohydrochloride, 10 mM L-Methionine, and 0.02% (w/v) PS20, at pH 5.5, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL. In a further embodiment, provided herein is a pharmaceutical composition comprising an anti-CD30L antibody or antigen binding fragment selected from those provided in Section 4.1, 20 mM histidine buffer, 100 mM arginine monohydrochloride, 10 mM L-Methionine, and 0.02% (w/v) PS20, at pH 5.5, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL.

In one embodiment, provided herein is a pharmaceutical composition comprising an anti-CD30L antibody or antigen binding fragment at a concentration of 200 mg/mL, 20 mM histidine buffer, 100 mM arginine monohydrochloride, 10 mM L-Methionine, and 0.02% (w/v) PS20, at pH 5.5, wherein the anti-CD30L comprises a CDR-H1 comprising the amino acid sequence set forth in any one of SEQ ID NOs: 110-119, 629, 636, and 712-723, a CDR-H2 comprising the amino acid sequence set forth in any one of SEQ ID NOs: 150-159, 643, 650, and 724-735, a CDR-H3 comprising the amino acid sequence set forth in any one of SEQ ID NOs: 190-199, 657, 664, and 736-743, a CDR-L1 comprising the amino acid sequence set forth in any one of SEQ ID NOs: 310-319, 671, 678, and 744-751, a CDR-L2 comprising the amino acid sequence set forth in any one of SEQ ID NOs: 350-359, 685, 692, and 752-759, and a CDR-L3 comprising the amino acid sequence set forth in any one of SEQ ID NOs: 390-399, 699, 706, and 760-765. In yet another embodiment, provided herein is a pharmaceutical composition comprising an anti-CD30L antibody or antigen binding fragment, 20 mM histidine buffer, 100 mM arginine monohydrochloride, 10 mM L-Methionine, and 0.02% (w/v) PS20, at pH 5.5, wherein the concentration of the anti-CD30L is about or greater than about 155, 160, 165, 170, 175, 180, 185, 190, 195, 200, 205, 210, 215, 220, 225, 230, 235, 240, 245, or 250 mg/mL, and wherein the anti-CD30L comprises a CDR-H1 comprising the amino acid sequence set forth in any one of SEQ ID NOs: 110-119, 629, 636, and 712-723, a CDR-H2 comprising the amino acid sequence set forth in any one of SEQ ID NOs: 150-159, 643, 650, and 724-735, a CDR-H3 comprising the amino acid sequence set forth in any one of SEQ ID NOs: 190-199, 657, 664, and 736-743, a CDR-L1 comprising the amino acid sequence set forth in any one of SEQ ID NOs: 310-319, 671, 678, and 744-751, a CDR-L2 comprising the amino acid sequence set forth in any one of SEQ ID NOs: 350-359, 685, 692, and 752-759, and a CDR-L3 comprising the amino acid sequence set forth in any one of SEQ ID NOs: 390-399, 699, 706, and 760-765.

Also described herein are kits comprising one or more of the anti-CD30L antibodies described herein in a suitable container and one or more additional components selected from: instructions for use; a diluent, an excipient, a carrier, and a device for administration.

### 4.6 Methods of Treatment

The pharmaceutical composition of the anti-CD30L antibodies or antigen binding fragments thereof described herein are useful in various embodiments for treating or ameliorating an autoimmune disorder in an individual. For example, in some embodiments, the pharmaceutical composition of the anti-CD30L antibodies or antigen binding fragments thereof are useful in methods of treating an autoimmune disorder in an individual, wherein the method comprises administering the pharmaceutical composition of the anti-CD30L antibodies or antigen binding fragments thereof to the individual. Accordingly, provided methods of treating an autoimmune disorder in an individual, wherein the method comprises administering the pharmaceutical composition of the anti-CD30L antibodies or antigen binding fragments thereof to the individual, thereby treating or meliorating the immune disorder. In certain embodiments, the autoimmune disorder is inflammatory bowel disease (IBD). In certain embodiments, the inflammatory bowel disease (IBD) is Crohn's disease (CD), ulcerative colitis (UC), and/or fibrosis. In certain instances, Inflammatory bowel disease (IBD) refers to a chronic disorder characterized by inflammation of the gastrointestinal tract. IBD encompasses and/or includes ulcerative colitis, which affects the large intestine and/or rectum, and Crohn's disease, which can affect the entire gastrointestinal system or be localized to the small intestine (ileum) and regions of the large intestine.

The terms “patient” “individual” or “subject” are used interchangeably and refer to any animal, including, but not limited to, humans, non-human primates, rodents, and domestic and game animals, which is to be the recipient of a particular treatment. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In various embodiments, a subject can be one who has been previously diagnosed with or identified as suffering from or having a condition in need of treatment. In certain embodiments, the subject is a human. In various other embodiments, the subject previously diagnosed with or identified as suffering from or having a condition may or may not have undergone treatment for a condition. In yet other embodiments, a subject can also be one who has not been previously diagnosed as having a condition (i.e., a subject who exhibits one or more risk factors for a condition). An “individual in need” of treatment for a particular condition can be a subject having that condition, diagnosed as having that condition, or at risk of developing that condition. In some embodiments, the individual is a “patient,” that has been diagnosed with a disease or condition described herein.

In certain embodiments, administering the pharmaceutical composition of the anti-CD30L antibodies or antigen binding fragments thereof comprises administering a therapeutically effective amount of the recombinant anti-CD30L antibodies or antigen binding fragments thereof. In certain instances, the term “therapeutically effective amount” refers to an amount of an antibody, polypeptide, polynucleotide, small organic molecule, or other drug effective to “treat” a disease or disorder in a subject or mammal. In certain instances, therapeutically effective amount of the drug reduces the severity of symptoms of the disease or disorder. In some embodiments, the disease or disorder comprises inflammatory bowel disease (IBD), Crohn's disease (CD), or ulcerative colitis (UC). In certain embodiments, the IBD, CD, and/or UC are severe or medically refractory forms of the IBD, CD, and/or UC. Non-limiting examples of symptoms of IBD, CD, and/or UC include, but are not limited to, diarrhea, fever, fatigue, abdominal pain, abdominal cramping, inflammation, ulceration, nausea, vomiting, bleeding, blood in stool, reduced appetite, and weight loss.

In certain instances, the terms, “treat” or “treating” as used herein refer to both therapeutic treatment and prophylactic or preventative measures (e.g., disease progression), wherein the object is to prevent or slow down (lessen) the targeted pathologic condition. In some embodiments provided herein, individuals in need of treatment include those already with a disease or condition, as well as those susceptible to develop the disease or condition. In additional embodiments, the disease or condition comprises an inflammatory disease or autoimmune condition.

The pharmaceutical composition of the anti-CD30L antibodies or antigen binding fragments thereof described herein are further useful in various embodiments for inhibiting, reducing, and/or preventing an inflammatory immune response or immune activation in an individual, wherein the method comprises administering the pharmaceutical composition of the anti-CD30L antibodies or antigen binding fragments thereof. In certain instances, the inflammatory immune response or immune activation comprises the expression, release, and/or activation of cytokine molecules and signaling pathways. In certain instances, cytokine is a generic term for proteins released by one cell population which act on another cell as intercellular mediators. Examples of such cytokines are lymphokines, monokines, and traditional polypeptide hormones. Included among the cytokines are transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1a, IL-2, IL-3, IL-4, IL-5, IL-6, IL-7, IL-8, IL-9, IL-10, IL-11, IL-12, IL-13, IL-17, IL-21, IL-22, IL-26; a tumor necrosis factor such as TNF-α or TNF-β; and other polypeptide factors including LIF and kit ligand (KL). The anti-CD30L antibodies are useful for reducing the expression/secretion of pro-inflammatory cytokines such as, for example, IL-1, IL-1α, IL-2, IL-6, IL-7, IL-8, IL-12, IL-23, interferon-α, -β, and -γ, and GM-CSF. Without being bound by theory, pro-inflammatory cytokines are released by inflammatory T cell subsets such as Tₕ1 or Tₕ17 cells, which play a role in many immune mediated pathologies including inflammatory bowel disease, ulcerative colitis, and Crohn's disease. Thus, effective reduction or blockade of these pro-inflammatory cytokines or interleukins addresses a key etiology of many autoimmune and inflammatory disorders. In another instance, the inflammatory immune response or immune activation comprises apoptosis of cells affected by an autoimmune disorder. In another instance, the inflammatory immune response or immune activation comprises the presence or activation of immune cells (e.g. macrophages, T cells, T helper cells, natural killer cells, etc.) in a tissue affected by an autoimmune disorder. In certain embodiments, immune cells encompass any cell derived from a hematopoietic stem cell that plays a role in the immune response. Immune cells include, without limitation, lymphocytes, such as T cells and B cells, antigen-presenting cells (APC), dendritic cells, monocytes, macrophages, natural killer (NK) cells, mast cells, basophils, eosinophils, or neutrophils, as well as any progenitors of such cells. In certain embodiments, the immune cell is a T cell. In certain embodiments, T cells include all cells within the T cell lineage, including thymocytes, immature T cells, mature T cells and the like. Thus, T cells include CD4⁺ T cells, CD8⁺ T cells, T helper (Tₕ) cells (for example, Tₕ1, Tₕ2 and Tₕ17 cells) and T regulatory (T_{reg}) cells.

Accordingly, provided herein are methods of inhibiting, reducing, and/or preventing an inflammatory immune response or immune activation in an individual comprising administering the pharmaceutical composition of the anti-CD30L antibody or antigen binding fragment described herein. In some embodiments, the inflammatory immune response or immune activation is characterized by the presence or activation of cytokine signaling molecules, the presence or activation of cellular apoptosis, and/or the presence or activation of immune cells. In certain embodiments, the presence or activation of cytokine signaling molecules, the presence or activation of cellular apoptosis, and/or the presence or activation of immune cells is the result of an autoimmune disorder. In certain embodiments, the presence or activation of cytokine signaling molecules, the presence or activation of cellular apoptosis, and/or the presence or activation of immune cells is localized to a specific tissue affected by the autoimmune disorder. In certain embodiments, the autoimmune disease or disorder comprises inflammatory bowel disease (IBD), Crohn's disease (CD), or ulcerative colitis (UC). In certain embodiments, the IBD, CD, and/or UC are severe or medically refractory forms of the IBD, CD, and/or UC. In some embodiments of the methods provided herein, including in this paragraph, the pharmaceutical composition is selected from the pharmaceutical compositions of Sections 2 and 4.5. In one embodiment of the methods provided herein, including in this paragraph, the anti-CD30L antibody or antigen binding fragment thereof in the pharmaceutical composition is selected from group consisting of the anti-CD30L antibodies or antigen binding fragments of Sections 2 and 4.1.

Furthermore, the pharmaceutical composition of the anti-CD30L antibodies or antigen binding fragments thereof described herein are useful for inhibiting and/or reducing binding of a CD30L molecule to a CD30 molecule. In some embodiments, the pharmaceutical composition of the anti-CD30L antibodies or antigen binding fragments thereof described herein are useful for preventing, inhibiting and/or reducing binding of a CD30L molecule to a CD30 molecule in an individual. In some embodiments, inhibiting and/or reducing binding of a CD30L molecule to a CD30 molecule prevents, reduces, or inhibits an inflammatory immune response and/or the activation of immune cells, as described herein. Accordingly, provided are method of inhibiting and/or reducing binding of a CD30L molecule to a CD30 molecule, wherein the method comprises administering the pharmaceutical composition of the anti-CD30L antibodies or antigen binding fragments thereof described herein to the individual. In certain embodiments, the individual comprises an autoimmune disorder. In certain embodiments, the autoimmune disorder comprises inflammatory bowel disease (IBD), Crohn's disease (CD), or ulcerative colitis (UC). In certain embodiments, the IBD, CD, and/or UC are severe or medically refractory forms of the IBD, CD, and/or UC. In some embodiments of the methods provided herein, including in this paragraph, the pharmaceutical composition is selected from the pharmaceutical compositions of Sections 2 and 4.5. In one embodiment of the methods provided herein, including in this paragraph, the anti-CD30L antibody or antigen binding fragment thereof in the pharmaceutical composition is selected from group consisting of the anti-CD30L antibodies or antigen binding fragments of Sections 2 and 4.1.

As is clear from the descriptions above and below, the pharmaceutical compositions and the anti-CD30L antibodies or antigen binding fragments provided herein can be used in various methods of treatment. Accordingly, in one aspect, provided herein is a method of treating or ameliorating an autoimmune disease in a subject in need thereof, the method comprising administering to the subject a pharmaceutical composition selected from the pharmaceutical compositions of Sections 2 and 4.5, thereby treating or ameliorating the autoimmune disease. In one embodiment of the methods of this paragraph, the autoimmune disease is irritable bowel disease. In another embodiment of the methods of this paragraph, the irritable bowel disease comprises ulcerative colitis (UC) or Crohn's Disease (CD). In one embodiment of the methods of this paragraph, the subject is a human. In some embodiments of the methods of this paragraph, the subject has fibrosis. In one embodiment of the methods of this paragraph, the anti-CD30L antibody or antigen binding fragment thereof in the pharmaceutical composition is selected from group consisting of the anti-CD30L antibodies or antigen binding fragments of Sections 2 and 4.1. In another embodiment of the methods of this paragraph, the anti-CD30L antibody or antigen binding fragment thereof in the pharmaceutical composition binds to an epitope comprising one or more amino acids in CD30L selected from the group consisting of D118, N165, K166, H167, I168, K169, S217, and D234, wherein the amino acids are numbered according to the amino acid sequence of CD30L as set forth in SEQ ID NO:33. Examples of the epitope comprising one or more (e.g. 1, 2, 3, 4, 5, 6, 7, or 8) amino acids in CD30L selected from the group consisting of D118, N165, K166, H167, I168, K169, S217, and D234 are described in details in Section 4.1, which have already been provided as embodiments for the methods of this Section and this paragraph.

Additionally, in one aspect, provided herein is a method for inhibiting and/or reducing binding of CD30L to CD30 in a subject afflicted with an inflammatory or autoimmune disorder, the method comprising administering to the subject afflicted with the inflammatory or autoimmune disorder a pharmaceutical composition selected from the pharmaceutical compositions of Sections 2 and 4.5, thereby inhibiting and/or reducing the binding of CD30L to CD30. In one embodiment of the methods of this paragraph, the subject is afflicted with an autoimmune disease. In another embodiment of the methods of this paragraph, the autoimmune disease is irritable bowel disease. In yet another embodiment of the methods of this paragraph, the irritable bowel disease comprises ulcerative colitis (UC) or Crohn's Disease (CD). In one embodiment of the methods of this paragraph, the subject is a human. In some embodiments of the methods of this paragraph, the subject has fibrosis. In one embodiment of the methods of this paragraph, the anti-CD30L antibody or antigen binding fragment thereof in the pharmaceutical composition is selected from group consisting of the anti-CD30L antibodies or antigen binding fragments of Sections 2 and 4.1. In another embodiment of the methods of this paragraph, the anti-CD30L antibody or antigen binding fragment thereof in the pharmaceutical composition binds to an epitope comprising one or more amino acids in CD30L selected from the group consisting of D118, N165, K166, H167, I168, K169, S217, and D234, wherein the amino acids are numbered according to the amino acid sequence of CD30L as set forth in SEQ ID NO:33. Examples of the epitope comprising one or more (e.g. 1, 2, 3, 4, 5, 6, 7, or 8) amino acids in CD30L selected from the group consisting of D118, N165, K166, H167, I168, K169, S217, and D234 are described in details in Section 4.1, which have already been provided as embodiments for the methods of this Section and this paragraph.

Alternatively, in another aspect, provided herein is a method of reducing and/or inhibiting inflammation in a subject, the method comprising administering to the subject a pharmaceutical composition selected from the pharmaceutical compositions of Sections 2 and 4.5, thereby reducing and/or inhibiting inflammation. In one embodiment of the methods of this paragraph, reducing and/or inhibiting inflammation comprises reducing an amount of pro-inflammatory cytokine expression or secretion in the subject or a tissue of the subject. In another embodiment of the methods of this paragraph, reducing and/or inhibiting inflammation comprises reducing an amount of pro-inflammatory cytokine expression or secretion by a T lymphocyte of the subject. In a further embodiment of the methods of this paragraph, the pro-inflammatory cytokine comprises interleukin-8. In yet another embodiment of the methods of this paragraph, the subject has an autoimmune disease. In one embodiment of the methods of this paragraph, the autoimmune disease is irritable bowel disease. In another embodiment of the methods of this paragraph, the irritable bowel disease comprises ulcerative colitis (UC) or Crohn's Disease (CD). In one embodiment of the methods of this paragraph, the subject is a human. In some embodiments of the methods of this paragraph, the subject has fibrosis. In one embodiment of the methods of this paragraph, the anti-CD30L antibody or antigen binding fragment thereof in the pharmaceutical composition is selected from group consisting of the anti-CD30L antibodies or antigen binding fragments of Sections 2 and 4.1. In another embodiment of the methods of this paragraph, the anti-CD30L antibody or antigen binding fragment thereof in the pharmaceutical composition binds to an epitope comprising one or more amino acids in CD30L selected from the group consisting of D118, N165, K166, H167, I168, K169, S217, and D234, wherein the amino acids are numbered according to the amino acid sequence of CD30L as set forth in SEQ ID NO:33. Examples of the epitope comprising one or more (e.g. 1, 2, 3, 4, 5, 6, 7, or 8) amino acids in CD30L selected from the group consisting of D118, N165, K166, H167, I168, K169, S217, and D234 are described in details in Section 4.1, which have already been provided as embodiments for the methods of this Section and this paragraph.

Specifically, in one aspect, provided herein is a method of treating or ameliorating an autoimmune disease in a subject in need thereof, the method comprising administering to the subject a pharmaceutical composition, wherein the pharmaceutical composition comprises an anti-CD30L antibody or antigen binding fragment thereof that binds isoform 1 of CD30L and does not bind to isoform 2 of CD30L, thereby treating or ameliorating the autoimmune disease. In one embodiment of the methods of this paragraph, the autoimmune disease is irritable bowel disease. In another embodiment of the methods of this paragraph, the irritable bowel disease comprises ulcerative colitis (UC) or Crohn's Disease (CD). In yet another embodiment of the methods of this paragraph, the isoform 1 of CD30L comprises the amino acid sequence set forth in SEQ ID NO: 34 and the isoform 2 of CD30L comprises the amino acid sequence set forth in SEQ ID NO: 35. In one embodiment of the methods of this paragraph, the subject is a human. In some embodiments of the methods of this paragraph, the subject has fibrosis. In one embodiment of the methods of this paragraph, the anti-CD30L antibody or antigen binding fragment thereof in the pharmaceutical composition is selected from group consisting of the anti-CD30L antibodies or antigen binding fragments of Sections 2 and 4.1. In another embodiment of the methods of this paragraph, the anti-CD30L antibody or antigen binding fragment thereof in the pharmaceutical composition binds to an epitope comprising one or more amino acids in CD30L selected from the group consisting of D118, N165, K166, H167, I168, K169, S217, and D234, wherein the amino acids are numbered according to the amino acid sequence of CD30L as set forth in SEQ ID NO:33. Examples of the epitope comprising one or more (e.g. 1, 2, 3, 4, 5, 6, 7, or 8) amino acids in CD30L selected from the group consisting of D118, N165, K166, H167, I168, K169, S217, and D234 are described in details in Section 4.1, which have already been provided as embodiments for the methods of this Section and this paragraph.

Similarly, in one aspect, provided herein is a method of specifically inhibiting and/or specifically reducing binding of CD30L isoform 1 to CD30 in a subject in need thereof, the method comprising administering to the subject a pharmaceutical composition, wherein the pharmaceutical composition comprises an anti-CD30L antibody or antigen binding fragment thereof that binds isoform 1 of CD30L and does not bind to isoform 2 of CD30L, thereby specifically inhibiting and/or specifically reducing binding of CD30L isoform 1 to CD30 in the subject. In one embodiment of the methods of this paragraph, the subject is afflicted with an autoimmune disease. In one embodiment of the methods of this paragraph, the autoimmune disease is irritable bowel disease. In another embodiment of the methods of this paragraph, the irritable bowel disease comprises ulcerative colitis (UC) or Crohn's Disease (CD). In yet another embodiment of the methods of this paragraph, the isoform 1 of CD30L comprises the amino acid sequence set forth in SEQ ID NO: 34 and the isoform 2 of CD30L comprises the amino acid sequence set forth in SEQ ID NO: 35. In one embodiment of the methods of this paragraph, the subject is a human. In some embodiments of the methods of this paragraph, the subject has fibrosis. In one embodiment of the methods of this paragraph, the anti-CD30L antibody or antigen binding fragment thereof in the pharmaceutical composition is selected from group consisting of the anti-CD30L antibodies or antigen binding fragments of Sections 2 and 4.1. In another embodiment of the methods of this paragraph, the anti-CD30L antibody or antigen binding fragment thereof in the pharmaceutical composition binds to an epitope comprising one or more amino acids in CD30L selected from the group consisting of D118, N165, K166, H167, I168, K169, S217, and D234, wherein the amino acids are numbered according to the amino acid sequence of CD30L as set forth in SEQ ID NO:33. Examples of the epitope comprising one or more (e.g. 1, 2, 3, 4, 5, 6, 7, or 8) amino acids in CD30L selected from the group consisting of D118, N165, K166, H167, I168, K169, S217, and D234 are described in details in Section 4.1, which have already been provided as embodiments for the methods of this Section and this paragraph.

Alternatively, in one aspect, provided herein is a method of treating or ameliorating an autoimmune disease in a subject in need thereof, the method comprising specifically inhibiting and/or specifically reducing binding of CD30L isoform 1 to CD30 in the subject with a pharmaceutical composition comprising an anti-CD30L antibody or antigen binding fragment thereof that binds isoform 1 of CD30L and does not bind to isoform 2 of CD30L. In another aspect, provided herein is a method of treating or ameliorating an autoimmune disease in a subject in need thereof, the method comprising (a) administering to the subject a pharmaceutical composition, wherein the pharmaceutical composition comprises an anti-CD30L antibody or antigen binding fragment thereof that binds isoform 1 of CD30L and does not bind to isoform 2 of CD30L, (b) specifically inhibiting and/or specifically reducing binding of CD30L isoform 1 to CD30 in the subject. In yet another aspect, provided herein is a method of treating or ameliorating an autoimmune disease in a subject in need thereof, the method comprising administering to the subject a pharmaceutical composition, wherein the pharmaceutical composition comprises an anti-CD30L antibody or antigen binding fragment thereof that binds isoform 1 of CD30L and does not bind to isoform 2 of CD30L, thereby specifically inhibiting and/or specifically reducing binding of CD30L isoform 1 to CD30 in the subject. In one embodiment of the methods of this paragraph, the autoimmune disease is irritable bowel disease. In another embodiment of the methods of this paragraph, the irritable bowel disease comprises ulcerative colitis (UC) or Crohn's Disease (CD). In yet another embodiment of the methods of this paragraph, the isoform 1 of CD30L comprises the amino acid sequence set forth in SEQ ID NO: 34 and the isoform 2 of CD30L comprises the amino acid sequence set forth in SEQ ID NO: 35. In one embodiment of the methods of this paragraph, the subject is a human. In some embodiments of the methods of this paragraph, the subject has fibrosis. In one embodiment of the methods of this paragraph, the anti-CD30L antibody or antigen binding fragment thereof in the pharmaceutical composition is selected from group consisting of the anti-CD30L antibodies or antigen binding fragments of Sections 2 and 4.1. In another embodiment of the methods of this paragraph, the anti-CD30L antibody or antigen binding fragment thereof in the pharmaceutical composition binds to an epitope comprising one or more amino acids in CD30L selected from the group consisting of D118, N165, K166, H167, I168, K169, S217, and D234, wherein the amino acids are numbered according to the amino acid sequence of CD30L as set forth in SEQ ID NO:33. Examples of the epitope comprising one or more (e.g. 1, 2, 3, 4, 5, 6, 7, or 8) amino acids in CD30L selected from the group consisting of D118, N165, K166, H167, I168, K169, S217, and D234 are described in details in Section 4.1, which have already been provided as embodiments for the methods of this Section and this paragraph.

As already described above, the anti-CD30L antibodies or antigen binding fragments provided herein can also be used in various methods of treatment. Accordingly, in one aspect, provided herein is a method of treating or ameliorating an autoimmune disease in a subject in need thereof, the method comprising administering to the subject an anti-CD30L antibody or antigen binding fragment selected from the anti-CD30L antibodies and antigen binding fragments of Sections 2 and 4.1, thereby treating or ameliorating the autoimmune disease. In one embodiment of the methods of this paragraph, the autoimmune disease is irritable bowel disease. In another embodiment of the methods of this paragraph, the irritable bowel disease comprises ulcerative colitis (UC) or Crohn's Disease (CD). In one embodiment of the methods of this paragraph, the subject is a human. In some embodiments of the methods of this paragraph, the subject has fibrosis. In another embodiment of the methods of this paragraph, the anti-CD30L antibody or antigen binding fragment thereof binds to an epitope comprising one or more amino acids in CD30L selected from the group consisting of D118, N165, K166, H167, I168, K169, S217, and D234, wherein the amino acids are numbered according to the amino acid sequence of CD30L as set forth in SEQ ID NO: 33. Examples of the epitope comprising one or more (e.g. 1, 2, 3, 4, 5, 6, 7, or 8) amino acids in CD30L selected from the group consisting of D118, N165, K166, H167, I168, K169, S217, and D234 are described in details in Section 4.1, which have already been provided as embodiments for the methods of this Section and this paragraph.

Additionally, in one aspect, provided herein is a method for inhibiting and/or reducing binding of CD30L to CD30 in a subject afflicted with an inflammatory or autoimmune disorder, the method comprising administering to the subject afflicted with the inflammatory or autoimmune disorder an anti-CD30L antibody or antigen binding fragment selected from the anti-CD30L antibodies and antigen binding fragments of Sections 2 and 4.1, thereby inhibiting and/or reducing the binding of CD30L to CD30. In one embodiment of the methods of this paragraph, the subject is afflicted with an autoimmune disease. In another embodiment of the methods of this paragraph, the autoimmune disease is irritable bowel disease. In yet another embodiment of the methods of this paragraph, the irritable bowel disease comprises ulcerative colitis (UC) or Crohn's Disease (CD). In one embodiment of the methods of this paragraph, the subject is a human. In some embodiments of the methods of this paragraph, the subject has fibrosis. In another embodiment of the methods of this paragraph, the anti-CD30L antibody or antigen binding fragment thereof binds to an epitope comprising one or more amino acids in CD30L selected from the group consisting of D118, N165, K166, H167, I168, K169, S217, and D234, wherein the amino acids are numbered according to the amino acid sequence of CD30L as set forth in SEQ ID NO: 33. Examples of the epitope comprising one or more (e.g. 1, 2, 3, 4, 5, 6, 7, or 8) amino acids in CD30L selected from the group consisting of D118, N165, K166, H167, I168, K169, S217, and D234 are described in details in Section 4.1, which have already been provided as embodiments for the methods of this Section and this paragraph.

Alternatively, in another aspect, provided herein is a method of reducing and/or inhibiting inflammation in a subject, the method comprising administering to the subject an anti-CD30L antibody or antigen binding fragment selected from the anti-CD30L antibodies and antigen binding fragments of Sections 2 and 4.1, thereby reducing and/or inhibiting inflammation. In one embodiment of the methods of this paragraph, reducing and/or inhibiting inflammation comprises reducing an amount of pro-inflammatory cytokine expression or secretion in the subject or a tissue of the subject. In another embodiment of the methods of this paragraph, reducing and/or inhibiting inflammation comprises reducing an amount of pro-inflammatory cytokine expression or secretion by a T lymphocyte of the subject. In a further embodiment of the methods of this paragraph, the pro-inflammatory cytokine comprises interleukin-8. In yet another embodiment of the methods of this paragraph, the subject has an autoimmune disease. In one embodiment of the methods of this paragraph, the autoimmune disease is irritable bowel disease. In another embodiment of the methods of this paragraph, the irritable bowel disease comprises ulcerative colitis (UC) or Crohn's Disease (CD). In one embodiment of the methods of this paragraph, the subject is a human. In some embodiments of the methods of this paragraph, the subject has fibrosis. In another embodiment of the methods of this paragraph, the anti-CD30L antibody or antigen binding fragment thereof binds to an epitope comprising one or more amino acids in CD30L selected from the group consisting of D118, N165, K166, H167, I168, K169, S217, and D234, wherein the amino acids are numbered according to the amino acid sequence of CD30L as set forth in SEQ ID NO:33. Examples of the epitope comprising one or more (e.g. 1, 2, 3, 4, 5, 6, 7, or 8) amino acids in CD30L selected from the group consisting of D118, N165, K166, H167, I168, K169, S217, and D234 are described in details in Section 4.1, which have already been provided as embodiments for the methods of this Section and this paragraph.

Specifically, in one aspect, provided herein is a method of treating or ameliorating an autoimmune disease in a subject in need thereof, the method comprising administering to the subject an anti-CD30L antibody or antigen binding fragment thereof, wherein the anti-CD30L antibody or antigen binding fragment thereof binds isoform 1 of CD30L and does not bind to isoform 2 of CD30L, thereby treating or ameliorating the autoimmune disease. In one embodiment of the methods of this paragraph, the autoimmune disease is irritable bowel disease. In another embodiment of the methods of this paragraph, the irritable bowel disease comprises ulcerative colitis (UC) or Crohn's Disease (CD). In yet another embodiment of the methods of this paragraph, the isoform 1 of CD30L comprises the amino acid sequence set forth in SEQ ID NO: 34 and the isoform 2 of CD30L comprises the amino acid sequence set forth in SEQ ID NO: 35. In a further embodiment of the methods provided in this paragraph, the anti-CD30L antibody or antigen binding fragment thereof is selected from the anti-CD30L antibodies and antigen binding fragments of Sections 2 and 4.1. In one embodiment of the methods of this paragraph, the subject is a human. In some embodiments of the methods of this paragraph, the subject has fibrosis. In another embodiment of the methods of this paragraph, the anti-CD30L antibody or antigen binding fragment thereof binds to an epitope comprising one or more amino acids in CD30L selected from the group consisting of D118, N165, K166, H167, I168, K169, S217, and D234, wherein the amino acids are numbered according to the amino acid sequence of CD30L as set forth in SEQ ID NO:33. Examples of the epitope comprising one or more (e.g. 1, 2, 3, 4, 5, 6, 7, or 8) amino acids in CD30L selected from the group consisting of D118, N165, K166, H167, I168, K169, S217, and D234 are described in details in Section 4.1, which have already been provided as embodiments for the methods of this Section and this paragraph.

Similarly, in one aspect, provided herein is a method of specifically inhibiting and/or specifically reducing binding of CD30L isoform 1 to CD30 in a subject in need thereof, the method comprising administering to the subject an anti-CD30L antibody or antigen binding fragment thereof, wherein the anti-CD30L antibody or antigen binding fragment thereof binds isoform 1 of CD30L and does not bind to isoform 2 of CD30L, thereby specifically inhibiting and/or specifically reducing binding of CD30L isoform 1 to CD30 in the subject. In one embodiment of the methods of this paragraph, the subject is afflicted with an autoimmune disease. In one embodiment of the methods of this paragraph, the autoimmune disease is irritable bowel disease. In another embodiment of the methods of this paragraph, the irritable bowel disease comprises ulcerative colitis (UC) or Crohn's Disease (CD). In yet another embodiment of the methods of this paragraph, the isoform 1 of CD30L comprises the amino acid sequence set forth in SEQ ID NO: 34 and the isoform 2 of CD30L comprises the amino acid sequence set forth in SEQ ID NO: 35. In a further embodiment of the methods provided in this paragraph, the anti-CD30L antibody or antigen binding fragment thereof is selected from the anti-CD30L antibodies and antigen binding fragments of Sections 2 and 4.1. In one embodiment of the methods of this paragraph, the subject is a human. In some embodiments of the methods of this paragraph, the subject has fibrosis. In another embodiment of the methods of this paragraph, the anti-CD30L antibody or antigen binding fragment thereof binds to an epitope comprising one or more amino acids in CD30L selected from the group consisting of D118, N165, K166, H167, I168, K169, S217, and D234, wherein the amino acids are numbered according to the amino acid sequence of CD30L as set forth in SEQ ID NO:33. Examples of the epitope comprising one or more (e.g. 1, 2, 3, 4, 5, 6, 7, or 8) amino acids in CD30L selected from the group consisting of D118, N165, K166, H167, I168, K169, S217, and D234 are described in details in Section 4.1, which have already been provided as embodiments for the methods of this Section and this paragraph.

Alternatively, in one aspect, provided herein is a method of treating or ameliorating an autoimmune disease in a subject in need thereof, the method comprising specifically inhibiting and/or specifically reducing binding of CD30L isoform 1 to CD30 in the subject with an anti-CD30L antibody or antigen binding fragment thereof that binds isoform 1 of CD30L and does not bind to isoform 2 of CD30L. In another aspect, provided herein is a method of treating or ameliorating an autoimmune disease in a subject in need thereof, the method comprising (a) administering to the subject an anti-CD30L antibody or antigen binding fragment thereof, wherein the anti-CD30L antibody or antigen binding fragment thereof binds isoform 1 of CD30L and does not bind to isoform 2 of CD30L, (b) specifically inhibiting and/or specifically reducing binding of CD30L isoform 1 to CD30 in the subject. In yet another aspect, provided herein is a method of treating or ameliorating an autoimmune disease in a subject in need thereof, the method comprising administering to the subject an anti-CD30L antibody or antigen binding fragment thereof, wherein the anti-CD30L antibody or antigen binding fragment thereof binds isoform 1 of CD30L and does not bind to isoform 2 of CD30L, thereby specifically inhibiting and/or specifically reducing binding of CD30L isoform 1 to CD30 in the subject. In one embodiment of the methods of this paragraph, the autoimmune disease is irritable bowel disease. In another embodiment of the methods of this paragraph, the irritable bowel disease comprises ulcerative colitis (UC) or Crohn's Disease (CD). In yet another embodiment of the methods of this paragraph, the isoform 1 of CD30L comprises the amino acid sequence set forth in SEQ ID NO: 34 and the isoform 2 of CD30L comprises the amino acid sequence set forth in SEQ ID NO: 35. In a further embodiment of the methods provided in this paragraph, the anti-CD30L antibody or antigen binding fragment thereof is selected from the anti-CD30L antibodies and antigen binding fragments of Sections 2 and 4.1. In one embodiment of the methods of this paragraph, the subject is a human. In some embodiments of the methods of this paragraph, the subject has fibrosis. In another embodiment of the methods of this paragraph, the anti-CD30L antibody or antigen binding fragment thereof binds to an epitope comprising one or more amino acids in CD30L selected from the group consisting of D118, N165, K166, H167, I168, K169, S217, and D234, wherein the amino acids are numbered according to the amino acid sequence of CD30L as set forth in SEQ ID NO:33. Examples of the epitope comprising one or more (e.g. 1, 2, 3, 4, 5, 6, 7, or 8) amino acids in CD30L selected from the group consisting of D118, N165, K166, H167, I168, K169, S217, and D234 are described in details in Section 4.1, which have already been provided as embodiments for the methods of this Section and this paragraph.

Additionally, in yet another aspect, provided herein is a method of reducing and/or inhibiting inflammation in a subject, the method comprising specifically inhibiting and/or specifically reducing binding of CD30L isoform 1 to CD30 in the subject with an anti-CD30L antibody or antigen binding fragment thereof that binds isoform 1 of CD30L and does not bind to isoform 2 of CD30L. In another aspect, provided herein is a method of reducing and/or inhibiting inflammation in a subject, the method comprising (a) administering to the subject an anti-CD30L antibody or antigen binding fragment thereof, wherein the anti-CD30L antibody or antigen binding fragment thereof binds isoform 1 of CD30L and does not bind to isoform 2 of CD30L, (b) specifically inhibiting and/or specifically reducing binding of CD30L isoform 1 to CD30 in the subject. In yet another aspect, provided herein is a method of reducing and/or inhibiting inflammation in a subject, the method comprising administering to the subject an anti-CD30L antibody or antigen binding fragment thereof, wherein the anti-CD30L antibody or antigen binding fragment thereof binds isoform 1 of CD30L and does not bind to isoform 2 of CD30L, thereby specifically inhibiting and/or specifically reducing binding of CD30L isoform 1 to CD30 in the subject. In one embodiment of the methods of this paragraph, the autoimmune disease is irritable bowel disease. In another embodiment of the methods of this paragraph, the irritable bowel disease comprises ulcerative colitis (UC) or Crohn's Disease (CD). In yet another embodiment of the methods of this paragraph, the isoform 1 of CD30L comprises the amino acid sequence set forth in SEQ ID NO: 34 and the isoform 2 of CD30L comprises the amino acid sequence set forth in SEQ ID NO: 35. In a further embodiment of the methods provided in this paragraph, the anti-CD30L antibody or antigen binding fragment thereof is selected from the anti-CD30L antibodies or antigen binding fragments of Sections 2 and 4.1. In one embodiments of the methods of this paragraph, the subject is a human. In some embodiment of the methods of this paragraph, the subject has fibrosis. In another embodiment of the methods of this paragraph, the anti-CD30L antibody or antigen binding fragment thereof binds to an epitope comprising one or more amino acids in CD30L selected from the group consisting of D118, N165, K166, H167, I168, K169, S217, and D234, wherein the amino acids are numbered according to the amino acid sequence of CD30L as set forth in SEQ ID NO: 33. Examples of the epitope comprising one or more (e.g. 1, 2, 3, 4, 5, 6, 7, or 8) amino acids in CD30L selected from the group consisting of D118, N165, K166, H167, I168, K169, S217, and D234 are described in details in Section 4.1, which have already been provided as embodiments for the methods of this Section and this paragraph.

In certain embodiments of the methods provided in this Section, the subject has inflammation. In some embodiments of the methods provided in this Section, the subject has fibrosis. In some embodiment of the methods provided in this Section, the subject has both inflammation and fibrosis.

### 4.7 Methods of Making the Pharmaceutical Compositions

In another aspect, also provided is a method of making the various pharmaceutical compositions disclosed herein.

In certain embodiments, the method of making the various pharmaceutical compositions disclosed herein comprises: (a) culturing a cell in a medium, wherein the cell comprises one or more polynucleotides comprising nucleotide sequences encoding a heavy chain, a light chain, or both a heavy chain and a light chain of the antibody or antigen-binding fragment thereof provided herein.

In certain embodiments, the method of making the various pharmaceutical compositions disclosed herein further comprises: (b) expressing the one or more polynucleotides in the cell.

In certain embodiments, the method of making the various pharmaceutical compositions disclosed herein further comprises: (c) purifying the antibody or antigen-binding fragment thereof.

In certain embodiments, the method of making the various pharmaceutical compositions provided herein comprises: (a) culturing a cell in a medium, wherein the cell comprises one or more polynucleotides comprising nucleotide sequences encoding a heavy chain, a light chain, or both a heavy chain and a light chain of the antibody or antigen-binding fragment thereof provided herein; and (b) expressing the one or more polynucleotides in the cell. In other embodiments, the method of making the various pharmaceutical compositions comprises: (a) culturing a cell in a medium, wherein the cell comprises one or more polynucleotides comprising nucleotide sequences encoding a heavy chain, a light chain, or both a heavy chain and a light chain of the antibody or antigen-binding fragment thereof provided herein; (b) expressing the one or more polynucleotides in the cell; and (c) purifying the antibody or antigen-binding fragment thereof. In other embodiments, the method of making the various pharmaceutical compositions comprises: (a) culturing a cell in a medium, wherein the cell comprises one or more polynucleotides comprising nucleotide sequences encoding a heavy chain, a light chain, or both a heavy chain and a light chain of the antibody or antigen-binding fragment thereof provided herein; and (c) purifying the antibody or antigen-binding fragment thereof.

In certain embodiments, the method of making the various pharmaceutical compositions provided herein further comprises a formulation step.

In other embodiments, the method of making the various pharmaceutical compositions provided herein comprises: (a) culturing a cell in a medium, wherein the cell comprises one or more polynucleotides comprising nucleotide sequences encoding a heavy chain, a light chain, or both a heavy chain and a light chain of the antibody or antigen-binding fragment thereof provided herein; (b) expressing the one or more polynucleotides in the cell; (c) purifying the antibody or antigen-binding fragment thereof; and (d) formulating the antibody or antigen-binding fragment thereof as provided herein. In other embodiments, the method of making the various pharmaceutical compositions comprises: (a) culturing a cell in a medium, wherein the cell comprises one or more polynucleotides comprising nucleotide sequences encoding a heavy chain, a light chain, or both a heavy chain and a light chain of the antibody or antigen-binding fragment thereof provided herein; (c) purifying the antibody or antigen-binding fragment thereof; and (d) formulating the antibody or antigen-binding fragment thereof as provided herein.

The cells used herein can be any type of cell that a person of ordinary skill in the art uses in protein production (e.g., antibody production). In certain embodiments, the cell is CHO cell. In other embodiments, the cell is HEK293 cell. The medium used herein can be any suitable cell culture medium, including any suitable supplements, for the specific cell (e.g., CHO cell) used.

In certain embodiments of the methods, the purification steps comprise: (i) an affinity chromatography; (ii) a viral inactivation; (iii) an ion exchange chromatography; (iv) a viral filtration; and (v) an ultrafiltration/diafiltration.

In one embodiment, the affinity chromatography is a protein A affinity chromatography. In another embodiment, the viral inactivation step is a low-pH viral inactivation step. In yet another embodiment, the ion exchange chromatography is an anion exchange chromatography. In one embodiment, the affinity chromatography is a protein A affinity chromatography, and the viral inactivation step is a low-pH viral inactivation step. In another embodiment, the viral inactivation step is a low-pH viral inactivation step, and the ion exchange chromatography is an anion exchange chromatography. In yet another embodiment, the affinity chromatography is a protein A affinity chromatography, and the ion exchange chromatography is an anion exchange chromatography. In still another embodiment, the affinity chromatography is a protein A affinity chromatography, the viral inactivation step is a low-pH viral inactivation step, and the ion exchange chromatography is an anion exchange chromatography. Accordingly, in a specific embodiment of the methods, the purification steps comprise: (i) a protein A affinity chromatography; (ii) a low-pH viral inactivation step; (iii) an anion exchange chromatography; (iv) a viral filtration step; and (v) an ultrafiltration/diafiltration.

In another embodiment, the affinity chromatography is a protein G affinity chromatography. In yet another embodiment, the viral inactivation step can be solvent/detergent inactivation, pasteurization, or UV inactivation, or a combination thereof. In still another embodiment, the anion exchange chromatography can use any anion exchange media commercially available, such as Q or DEAE. In yet another embodiment, the anion exchange chromatography is a multimodal anion exchange chromatography.

Thus, in one embodiment, the method of making the various pharmaceutical compositions provided herein comprises: (a) culturing a cell in a medium, wherein the cell comprises one or more polynucleotides comprising nucleotide sequences encoding a heavy chain, a light chain, or both a heavy chain and a light chain of the antibody or antigen-binding fragment thereof provided herein; (b) expressing the one or more polynucleotides in the cell; and (c) purifying the antibody or antigen-binding fragment thereof, wherein the purification step comprises (i) a protein A affinity chromatography; (ii) a low-pH viral inactivation step; (iii) an anion exchange chromatography; (iv) a viral filtration step; and (v) an ultrafiltration/diafiltration; and (d) formulating the antibody or antigen-binding fragment thereof purified from step (c).

### 4.8 Additional Exemplary Embodiments

As described herein, provided are recombinant antibodies and/or antigen binding fragments thereof that binds CD30L, wherein the antibody or antigen binding fragment thereof comprises: (a) a CDR-H1 comprising the amino acid sequence set forth in any one of SEQ ID NOs: 100-139 or 220-234; (b) a CDR-H2 comprising the amino acid sequence set forth in any one of SEQ ID NOs: 140-179 or 235-249; (c) an immunoglobulin heavy chain CDR3 (CDR-H3) comprising the amino acid sequence set forth in any one of SEQ ID NOs: 180-219 or 250-264; (d) a CDR-L1 comprising the amino acid sequence set forth in any one of SEQ ID NOs: 300-339 or 420-434; (e) a CDR-L2 comprising the amino acid sequence set forth in any one of SEQ ID NOs: 340-379 or 435-449; and/or (f) a CDR-L3 comprising the amino acid sequence set forth in any one of SEQ ID NOs: 380-419 or 450-464.

In some embodiments, provided is an antibody or antigen binding fragment thereof of any of the preceding embodiments, wherein the antibody or antigen binding fragment thereof comprises: (a) a CDR-H1 comprising the amino acid sequence set forth in any one of SEQ ID NOs: 100-109; (b) a CDR-H2 comprising the amino acid sequence set forth in any one of SEQ ID NOs: 140-149; (c) an immunoglobulin heavy chain CDR3 (CDR-H3) comprising the amino acid sequence set forth in any one of SEQ ID NOs: 180-189; (d) a CDR-L1 comprising the amino acid sequence set forth in any one of SEQ ID NOs: 300-309; (e) a CDR-L2 comprising the amino acid sequence set forth in any one of SEQ ID NOs: 340-349; and/or (f) a CDR-L3 comprising the amino acid sequence set forth in any one of SEQ ID NOs: 380-389.

In some embodiments, provided is an antibody or antigen binding fragment thereof of any of the preceding embodiments, wherein the antibody or antigen binding fragment thereof comprises: (a) a CDR-H1 comprising the amino acid sequence set forth in any one of SEQ ID NOs: 100-104; (b) a CDR-H2 comprising the amino acid sequence set forth in any one of SEQ ID NOs: 140-144; (c) an immunoglobulin heavy chain CDR3 (CDR-H3) comprising the amino acid sequence set forth in any one of SEQ ID NOs: 180-184; (d) a CDR-L1 comprising the amino acid sequence set forth in any one of SEQ ID NOs: 300-304; (e) a CDR-L2 comprising the amino acid sequence set forth in any one of SEQ ID NOs: 340-344; and/or (f) a CDR-L3 comprising the amino acid sequence set forth in any one of SEQ ID NOs: 380-384.

In some embodiments, provided is an antibody or antigen binding fragment thereof of any of the preceding embodiments, wherein the antibody or antigen binding fragment thereof comprises: (a) a CDR-H1 comprising the amino acid sequence set forth in any one of SEQ ID NOs: 105-109; (b) a CDR-H2 comprising the amino acid sequence set forth in any one of SEQ ID NOs: 145-149; (c) an immunoglobulin heavy chain CDR3 (CDR-H3) comprising the amino acid sequence set forth in any one of SEQ ID NOs: 185-189; (d) a CDR-L1 comprising the amino acid sequence set forth in any one of SEQ ID NOs: 305-309; (e) a CDR-L2 comprising the amino acid sequence set forth in any one of SEQ ID NOs: 345-349; and/or (f) a CDR-L3 comprising the amino acid sequence set forth in any one of SEQ ID NOs: 385-389.

In some embodiments, provided is an antibody or antigen binding fragment thereof of any of the preceding embodiments, comprising an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to any one of SEQ ID NOs: 1 and 2; and/or (b) the immunoglobulin variable region light chain comprises an amino acid sequence having at least about 90, 95, 97, 98, 99, or 100% sequence identity to any one of SEQ ID NOs: 3 and 4.

In some embodiments, provided is an antibody or antigen binding fragment thereof of any of the preceding embodiments, wherein the antibody or antigen binding fragment thereof comprises: (a) a CDR-H1 comprising the amino acid sequence set forth in any one of SEQ ID NOs: 110-119; (b) a CDR-H2 comprising the amino acid sequence set forth in any one of SEQ ID NOs: 150-159; (c) an immunoglobulin heavy chain CDR3 (CDR-H3) comprising the amino acid sequence set forth in any one of SEQ ID NOs: 190-199; (d) a CDR-L1 comprising the amino acid sequence set forth in any one of SEQ ID NOs: 310-319; (e) a CDR-L2 comprising the amino acid sequence set forth in any one of SEQ ID NOs: 350-359; and/or (e) a CDR-L3 comprising the amino acid sequence set forth in any one of SEQ ID NOs: 390-399.

In some embodiments, provided is an antibody or antigen binding fragment thereof of any of the preceding embodiments, wherein the antibody or antigen binding fragment thereof comprises: (a) a CDR-H1 comprising the amino acid sequence set forth in any one of SEQ ID NOs: 110-114; (b) a CDR-H2 comprising the amino acid sequence set forth in any one of SEQ ID NOs: 150-154; (c) an immunoglobulin heavy chain CDR3 (CDR-H3) comprising the amino acid sequence set forth in any one of SEQ ID NOs: 190-194; (d) a CDR-L1 comprising the amino acid sequence set forth in any one of SEQ ID NOs: 310-314; (e) a CDR-L2 comprising the amino acid sequence set forth in any one of SEQ ID NOs: 350-354; and/or (e) a CDR-L3 comprising the amino acid sequence set forth in any one of SEQ ID NOs: 390-394.

In some embodiments, provided is an antibody or antigen binding fragment thereof of any of the preceding embodiments, wherein the antibody or antigen binding fragment thereof comprises: (a) a CDR-H1 comprising the amino acid sequence set forth in any one of SEQ ID NOs: 115-119; (b) a CDR-H2 comprising the amino acid sequence set forth in any one of SEQ ID NOs: 155-159; (c) an immunoglobulin heavy chain CDR3 (CDR-H3) comprising the amino acid sequence set forth in any one of SEQ ID NOs: 195-199; (d) a CDR-L1 comprising the amino acid sequence set forth in any one of SEQ ID NOs: 315-319; (e) a CDR-L2 comprising the amino acid sequence set forth in any one of SEQ ID NOs: 355-359; and/or (f) a CDR-L3 comprising the amino acid sequence set forth in any one of SEQ ID NOs: 395-399.

In some embodiments, provided is an antibody or antigen binding fragment thereof of any of the preceding embodiments, comprising an immunoglobulin variable region heavy chain and an immunoglobulin variable region light chain, wherein (a) the immunoglobulin variable region heavy chain comprises an amino acid sequence having at least about 90, 95, 97, 98, 99, or 100% sequence identity to any one of SEQ ID NOs: 5 and 6; and/or (b) the immunoglobulin variable region light chain comprises an amino acid sequence having at least about 90, 95, 97, 98, 99, or 100% sequence identity to any one of SEQ ID NOs: 7 and 8.

In some embodiments, provided is an antibody or antigen binding fragment thereof of any of the preceding embodiments, wherein the antibody or antigen binding fragment thereof comprises: (a) a CDR-H1 comprising the amino acid sequence set forth in any one of SEQ ID NOs: 120-129; (b) a CDR-H2 comprising the amino acid sequence set forth in any one of SEQ ID NOs: 160-169; (c) an immunoglobulin heavy chain CDR3 (CDR-H3) comprising the amino acid sequence set forth in any one of SEQ ID NOs: 200-209; (d) a CDR-L1 comprising the amino acid sequence set forth in any one of SEQ ID NOs: 320-329; (e) a CDR-L2 comprising the amino acid sequence set forth in any one of SEQ ID NOs: 360-369; and/or (f) a CDR-L3 comprising the amino acid sequence set forth in any one of SEQ ID NOs: 400-409.

In some embodiments, provided is an antibody or antigen binding fragment thereof of any of the preceding embodiments, wherein the antibody or antigen binding fragment thereof comprises: (a) a CDR-H1 comprising the amino acid sequence set forth in any one of SEQ ID NOs: 120-124; (b) a CDR-H2 comprising the amino acid sequence set forth in any one of SEQ ID NOs: 160-164; (c) an immunoglobulin heavy chain CDR3 (CDR-H3) comprising the amino acid sequence set forth in any one of SEQ ID NOs: 200-204; (d) a CDR-L1 comprising the amino acid sequence set forth in any one of SEQ ID NOs: 320-324; (e) a CDR-L2 comprising the amino acid sequence set forth in any one of SEQ ID NOs: 360-364; and/or (f) a CDR-L3 comprising the amino acid sequence set forth in any one of SEQ ID NOs: 400-404.

In some embodiments, provided is an antibody or antigen binding fragment thereof of any of the preceding embodiments, wherein the antibody or antigen binding fragment thereof comprises: (a) a CDR-H1 comprising the amino acid sequence set forth in any one of SEQ ID NOs: 125-129; (b) a CDR-H2 comprising the amino acid sequence set forth in any one of SEQ ID NOs: 165-169; (c) an immunoglobulin heavy chain CDR3 (CDR-H3) comprising the amino acid sequence set forth in any one of SEQ ID NOs: 205-209; (d) a CDR-L1 comprising the amino acid sequence set forth in any one of SEQ ID NOs: 325-329; (e) a CDR-L2 comprising the amino acid sequence set forth in any one of SEQ ID NOs: 365-369; and/or (f) a CDR-L3 comprising the amino acid sequence set forth in any one of SEQ ID NOs: 405-409.

In some embodiments, provided is an antibody or antigen binding fragment thereof of any of the preceding embodiments, comprising an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to any one of SEQ ID NOs: 9 and 10; and/or (b) the immunoglobulin variable region light chain comprises an amino acid sequence having at least about 90, 95, 97, 98, 99, or 100% sequence identity to any one of SEQ ID NOs: 11 and 12.

In some embodiments, provided is an antibody or antigen binding fragment thereof of any of the preceding embodiments, wherein the antibody or antigen binding fragment thereof comprises: (a) a CDR-H1 comprising the amino acid sequence set forth in any one of SEQ ID NOs: 130-139; (b) a CDR-H2 comprising the amino acid sequence set forth in any one of SEQ ID NOs: 170-179; (c) an immunoglobulin heavy chain CDR3 (CDR-H3) comprising the amino acid sequence set forth in any one of SEQ ID NOs: 210-219; (d) a CDR-L1 comprising the amino acid sequence set forth in any one of SEQ ID NOs: 330-339; (e) a CDR-L2 comprising the amino acid sequence set forth in any one of SEQ ID NOs: 370-379; and/or (f) a CDR-L3 comprising the amino acid sequence set forth in any one of SEQ ID NOs: 410-419.

In some embodiments, provided is an antibody or antigen binding fragment thereof of any of the preceding embodiments, wherein the antibody or antigen binding fragment thereof comprises: (a) a CDR-H1 comprising the amino acid sequence set forth in any one of SEQ ID NOs: 130-134; (b) a CDR-H2 comprising the amino acid sequence set forth in any one of SEQ ID NOs: 170-174; (c) an immunoglobulin heavy chain CDR3 (CDR-H3) comprising the amino acid sequence set forth in any one of SEQ ID NOs: 210-214; (d) a CDR-L1 comprising the amino acid sequence set forth in any one of SEQ ID NOs: 330-334; (e) a CDR-L2 comprising the amino acid sequence set forth in any one of SEQ ID NOs: 370-374; and/or (f) a CDR-L3 comprising the amino acid sequence set forth in any one of SEQ ID NOs: 410-414.

In some embodiments, provided is an antibody or antigen binding fragment thereof of any of the preceding embodiments, wherein the antibody or antigen binding fragment thereof comprises: (a) a CDR-H1 comprising the amino acid sequence set forth in any one of SEQ ID NOs: 135-139; (b) a CDR-H2 comprising the amino acid sequence set forth in any one of SEQ ID NOs: 175-179; (c) an immunoglobulin heavy chain CDR3 (CDR-H3) comprising the amino acid sequence set forth in any one of SEQ ID NOs: 215-219; (d) a CDR-L1 comprising the amino acid sequence set forth in any one of SEQ ID NOs: 335-339; (e) a CDR-L2 comprising the amino acid sequence set forth in any one of SEQ ID NOs: 375-379; and/or (f) a CDR-L3 comprising the amino acid sequence set forth in any one of SEQ ID NOs: 415-419.

In some embodiments, provided is an antibody or antigen binding fragment thereof of any of the preceding embodiments, comprising an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to any one of SEQ ID NOs: 13 and 14; and/or (b) the immunoglobulin variable region light chain comprises an amino acid sequence having at least about 90, 95, 97, 98, 99, or 100% sequence identity to any one of SEQ ID NOs: 15 and 16.

In some embodiments, provided is an antibody or antigen binding fragment thereof of any of the preceding embodiments, wherein the antibody or antigen binding fragment thereof comprises: (a) a CDR-H1 comprising the amino acid sequence set forth in any one of SEQ ID NOs: 220-224; (b) a CDR-H2 comprising the amino acid sequence set forth in any one of SEQ ID NOs: 235-239; (c) an immunoglobulin heavy chain CDR3 (CDR-H3) comprising the amino acid sequence set forth in any one of SEQ ID NOs: 250-254; (d) a CDR-L1 comprising the amino acid sequence set forth in any one of SEQ ID NOs: 420-424; (e) a CDR-L2 comprising the amino acid sequence set forth in any one of SEQ ID NOs: 435-439; and/or (f) a CDR-L3 comprising the amino acid sequence set forth in any one of SEQ ID NOs: 450-454.

In some embodiments, provided is an antibody or antigen binding fragment thereof of any of the preceding embodiments, comprising an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to any one of SEQ ID NO: 17; and/or (b) the immunoglobulin variable region light chain comprises an amino acid sequence having at least about 90, 95, 97, 98, 99, or 100% sequence identity to any one of SEQ ID NO: 15 and 18.

In some embodiments, provided is an antibody or antigen binding fragment thereof of any of the preceding embodiments, wherein the antibody or antigen binding fragment thereof comprises: (a) a CDR-H1 comprising the amino acid sequence set forth in any one of SEQ ID NOs: 225-229; (b) a CDR-H2 comprising the amino acid sequence set forth in any one of SEQ ID NOs: 240-244; (c) an immunoglobulin heavy chain CDR3 (CDR-H3) comprising the amino acid sequence set forth in any one of SEQ ID NOs: 255-259; (d) a CDR-L1 comprising the amino acid sequence set forth in any one of SEQ ID NOs: 425-429; (e) a CDR-L2 comprising the amino acid sequence set forth in any one of SEQ ID NOs: 440-444; and/or (f) a CDR-L3 comprising the amino acid sequence set forth in any one of SEQ ID NOs: 455-459.

In some embodiments, provided is an antibody or antigen binding fragment thereof of any of the preceding embodiments, comprising an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to any one of SEQ ID NO: 19; and/or (b) the immunoglobulin variable region light chain comprises an amino acid sequence having at least about 90, 95, 97, 98, 99, or 100% sequence identity to any one of SEQ ID NO: 15 and 20.

In some embodiments, provided is an antibody or antigen binding fragment thereof of any of the preceding embodiments, wherein the antibody or antigen binding fragment thereof comprises: (a) a CDR-H1 comprising the amino acid sequence set forth in any one of SEQ ID NOs: 230-234; (b) a CDR-H2 comprising the amino acid sequence set forth in any one of SEQ ID NOs: 245-249; (c) an immunoglobulin heavy chain CDR3 (CDR-H3) comprising the amino acid sequence set forth in any one of SEQ ID NOs: 260-264; (d) a CDR-L1 comprising the amino acid sequence set forth in any one of SEQ ID NOs: 430-434; (e) a CDR-L2 comprising the amino acid sequence set forth in any one of SEQ ID NOs: 445-449; and/or (f) a CDR-L3 comprising the amino acid sequence set forth in any one of SEQ ID NOs: 460-464.

In some embodiments, provided is an antibody or antigen binding fragment thereof of any of the preceding embodiments, comprising an immunoglobulin variable region heavy chain and an immunoglobulin variable region light chain, wherein: (a) the immunoglobulin variable region heavy chain comprises an amino acid sequence having at least about 90, 95, 97, 98, 99, or 100% sequence identity to any one of SEQ ID NO: 21; and/or (b) the immunoglobulin variable region light chain comprises an amino acid sequence having at least about 90, 95, 97, 98, 99, or 100% sequence identity to any one of SEQ ID NO: 15 and 22.

In some embodiments, provided is an antibody or antigen binding fragment thereof of any of the preceding embodiments, comprising a constant region (e.g. a fragment crystallizable (Fc) region) having reduced antibody-dependent cell-mediated cytotoxicity (ADCC) function as compared to human IgG1 and/or reduced complement-dependent cytotoxicity (CDC) as compared to human IgG1. In some embodiments, provided is an antibody or antigen binding fragment thereof of any of the preceding embodiments, wherein the Constant region comprises an amino acid sequence having 80, 85, 90, 95, 97, 98, 99, or 100% sequence identity to the amino acid sequence set forth by any one of SEQ ID NOs: 500-512. In some embodiments, provided is an antibody or antigen binding fragment thereof of any of the preceding embodiments, wherein the Constant region comprises the amino acid sequence set forth by any one of SEQ ID NOs: 500-512.

In some embodiments, provided is an antibody or antigen binding fragment thereof of any of the preceding embodiments, comprising a constant region having an amino acid sequence variant corresponding to (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(mmm), per EU numbering.

In some embodiments, provided is an antibody or antigen binding fragment thereof of any of the preceding embodiments, wherein the recombinant antibody or antigen binding fragment thereof is an IgG antibody. In some embodiments, provided is an antibody or antigen binding fragment thereof of any of the preceding embodiments, wherein the IgG antibody is IgG1, IgG2, IgG3, or IgG4. In some embodiments, provided is an antibody or antigen binding fragment thereof of any of the preceding embodiments, wherein the recombinant antibody or antigen binding fragment thereof is human, chimeric, or humanized.

In some embodiments, provided is an antibody or antigen binding fragment thereof of any of the preceding embodiments, wherein the recombinant antibody or antigen binding fragment thereof is a Fab, F(ab)′₂, a single-domain antibody, or a single chain variable fragment (scFv). In some embodiments, provided is an antibody or antigen binding fragment thereof of any of the preceding embodiments, wherein the recombinant antibody or antigen binding fragment thereof is a bispecific or multispecific antibody.

In some embodiments, provided is an antibody or antigen binding fragment thereof of any of the preceding embodiments, wherein the recombinant antibody or antigen binding fragment thereof inhibits a binding interaction between CD30L and CD30.

In an aspect, further provided are nucleic acids encoding the recombinant antibody or antigen binding fragment thereof of any of the preceding embodiments. Also provided are cells comprising the recombinant antibody or antigen binding fragment thereof of any of the preceding embodiments. In some embodiments, the cell is a eukaryotic cell. In some embodiments, the cell is a prokaryotic cell.

In an aspect, provided are recombinant antibodies or antigen binding fragments thereof of any the preceding embodiments for use in a method of inhibiting binding of CD30L to CD30. Further provided are recombinant antibodies or antigen binding fragments thereof of any the preceding embodiments for use in a method of inhibiting activation of CD30 signaling in a cell. Also provided are recombinant antibodies or antigen binding fragments thereof of any the preceding embodiments for use in a method of inhibiting activation, expression, and/or secretion of a pro-inflammatory cytokine protein.

In an aspect, provided are recombinant antibodies or antigen binding fragments thereof of any the preceding embodiments for use in treating an autoimmune disease in an individual in need thereof. In some embodiments, the autoimmune disease is irritable bowel disease. In some embodiments, the irritable bowel disease comprises ulcerative colitis (UC) or Crohn's Disease (CD).

In an aspect, provided are methods of treating or ameliorating an autoimmune disease in an individual in need thereof, the method comprising administering to the individual the recombinant antibody or antigen binding fragment thereof of any of the preceding embodiments, thereby treating or ameliorating the autoimmune disease. Also provided are methods for inhibiting and/or reducing binding of CD30L to CD30 in an individual with an inflammatory or autoimmune disorder, the method comprising administering to an individual afflicted with the inflammatory or autoimmune disorder the recombinant antibody or antigen binding fragment thereof of the preceding embodiments, thereby inhibiting and/or reducing the binding of CD30L to CD30. Further provided are methods of reducing and/or inhibiting inflammation in an individual, the method comprising administering to the individual the recombinant antibody or antigen binding fragment thereof of any of the preceding embodiments, thereby reducing and/or inhibiting inflammation.

In some embodiments, the provided are methods of any of the preceding embodiments, wherein the individual has an autoimmune disease. In some embodiments, the provided are methods of any of the preceding embodiments, wherein the autoimmune disease is irritable bowel disease. In some embodiments, the provided are methods of any of the preceding embodiments, wherein the irritable bowel disease comprises ulcerative colitis (UC) or Crohn's Disease (CD).

In some embodiments, the provided are methods of any of the preceding embodiments, wherein reducing and/or inhibiting inflammation comprises reducing an amount of pro-inflammatory cytokine expression or secretion in the individual or a tissue of the individual. In some embodiments, the provided are methods of any of the preceding embodiments, wherein the pro-inflammatory cytokine comprises interleukin 8 and/or interleukin 6. In some embodiments, the provided are methods of any of the preceding embodiments, wherein the individual has an autoimmune disease. In some embodiments, the provided are methods of any of the preceding embodiments, wherein the autoimmune disease is irritable bowel disease. In some embodiments, the provided are methods of any of the preceding embodiments, wherein the irritable bowel disease comprises ulcerative colitis (UC) or Crohn's Disease (CD).

### 5. EXAMPLES

The following examples are illustrative of the embodiments described herein and are not to be interpreted as limiting the scope of this disclosure. To the extent that specific materials are mentioned, it is merely for purposes of illustration and is not intended to be limiting. One skilled in the art may develop equivalent means or reactants without the exercise of inventive capacity and without departing from the scope of this disclosure.

### 5.1 Example 1: Generation and Identification of Anti-CD30L Antibodies

Mice with humanized immunoglobulin locus were used for antibody generation. Mice were inoculated with human CD30L expressing cells (B16 cell line transfected to express human CD30L), DNA encoding human CD30L, and recombinant human CD30L protein. At certain time points following the start of the immunizations, the serum of the mice was screened for reactivity to human CD30L and cynomolgus CD30L and plasma cells from mice with high titers were fused to generate hybridomas.

The hybridomas were then expanded, screened and selected based on their flow cytometry binding to cells expressing human and cyno CD30L, CD30:CD30L blocking activity when CD30L is expressed on cells by flow cytometry, ELISA binding to recombinant human CD30L protein, ELISA binding to recombinant cynomolgus CD30L protein, and CD30:CD30L blocking ability in an ELISA binding assay. Table 2, Table 3, and Table 4 show data from hybridoma screening assays. The anti-CD30L antibodies shown in Table 2 and Table 3 effectively bind recombinant CD30L, cells expressing CD30L, and inhibit binding of CD30L to CD30. Clone 1 comprises the CDRs as set forth in SEQ ID NOs: 100-104, 140-144, 180-184, 300-304, 340-344, and 380-384; Clone 2 comprises the CDRs as set forth in SEQ ID NOs: 110-114, 150-154, 190-194, 310-314, 350-354, and 390-394; Clone 3 comprises the CDRs as set forth in SEQ ID NOs: 120-124, 160-164, 200-204, 320-324, 360-364, and 400-404; and Clone 4 comprises the CDRs as set forth in SEQ ID NOs: 130-134, 170-174, 210-214, 330-334, 370-374, and 410-414; Clone 59 comprises the CDRs as set forth in SEQ ID NOs: 220-224, 235-239, 250-254, 420-424, 435-439, and 450-454; Clone 60 comprises the CDRs as set forth in SEQ ID NOs: 225-229, 240-244, 255-259, 425-429, 440-444, and 455-459; and Clone 61 comprises the CDRs as set forth in SEQ ID NOs: 230-234, 245-249, 260-264, 430-434, 445-449, and 460-464.

TABLE 2
Cell binding and ELISA screening against CD30L

| | Cell Binding (MFI) | | Elisa Binding (OD) | | |
|---|---|---|---|---|---|
| Clone | huCD30L | cyCD30L | huCD30L | huCD30L | huCD30L |
| ID | (1/50 dil) | (1/50 dil) | (1/50 dil) | (1/500 dil) | (1/5000 dil) |
| 1 | 208 | 77.3 | 3.586 | 3.025 | 1.283 |
| 2 | 280 | 57.6 | 2.147 | 1.712 | 0.565 |
| 3 | 153 | 72.4 | 3.495 | 2.54 | 0.662 |
| 4 | 151 | 67.8 | 3.075 | 2.652 | 0.68 |
| 5 | 205 | 79.6 | 3.166 | 2.849 | 1.111 |
| 6 | 200 | 61.9 | 2.374 | 1.377 | 0.503 |
| 7 | 234 | 77.6 | 2.17 | 1.404 | 0.395 |
| 8 | 186 | 69.9 | 2.255 | 1.074 | 0.255 |
| 9 | 234 | 62.8 | 1.829 | 1.202 | 0.358 |
| 10 | 192 | 74.6 | 2.14 | 1.233 | 0.313 |
| 11 | 176 | 68.4 | 2.294 | 1.274 | 0.324 |
| 12 | 186 | 76.7 | 3.067 | 2.679 | 0.822 |
| 13 | 168 | 69.6 | 3.244 | 2.797 | 0.825 |
| 14 | 168 | 70.3 | 2.045 | 1.129 | 0.27 |
| 15 | 176 | 67.1 | 2.951 | 2.6 | 0.754 |
| 16 | 176 | 76 | 2.014 | 1.133 | 0.284 |
| 17 | 166 | 68.3 | 3.184 | 2.448 | 0.719 |
| 18 | 171 | 70.1 | 3.183 | 2.571 | 0.774 |
| 19 | 137 | 57.5 | 1.87 | 0.93 | 0.221 |
| 20 | 197 | 79.8 | 2.085 | 1.345 | 0.3 |
| 21 | 160 | 66.2 | 2.302 | 1.239 | 0.295 |
| 22 | 156 | 74.6 | 3.128 | 2.673 | 0.82 |
| 23 | 170 | 64.2 | 3.482 | 2.494 | 0.701 |
| 24 | 170 | 68.7 | 3.176 | 2.618 | 0.697 |
| 25 | 155 | 64.2 | 3.104 | 2.411 | 0.621 |
| 26 | 167 | 66 | 2.919 | 2.48 | 0.658 |
| 27 | 149 | 68.8 | 3.206 | 2.395 | 0.605 |
| 28 | 144 | 68 | 3.046 | 2.342 | 0.569 |
| 29 | 139 | 65.9 | 3.158 | 2.306 | 0.588 |
| 30 | 154 | 62.9 | 3.137 | 2.389 | 0.645 |
| 31 | 141 | 68.2 | 3.043 | 2.316 | 0.589 |
| 32 | 149 | 68.2 | 3.012 | 2.397 | 0.608 |
| 33 | 132 | 68.5 | 3.059 | 2.224 | 0.536 |
| 34 | 115 | 57 | 1.923 | 0.784 | 0.177 |
| 35 | 148 | 57.4 | 3.18 | 2.429 | 0.572 |
| 36 | 146 | 57.7 | 3.288 | 2.308 | 0.582 |
| 37 | 127 | 57.2 | 1.826 | 0.834 | 0.193 |
| 38 | 146 | 63.1 | 2.864 | 2.31 | 0.628 |
| 39 | 135 | 52.4 | 2.004 | 0.846 | 0.178 |
| 40 | 123 | 50.4 | 1.198 | 0.791 | 0.191 |
| 41 | 120 | 51.9 | 0.502 | 0.349 | 0.081 |
| 42 | 104 | 53.1 | 3.109 | 1.928 | 0.408 |
| 43 | 104 | 51.7 | 1.772 | 0.643 | 0.142 |
| 44 | 76.2 | 46.4 | 1.545 | 0.52 | 0.114 |
| 45 | 0.54 | 0.54 | 0.314 | 0.085 | 0.047 |
| 46 | 0.74 | 0.77 | 0.454 | 0.111 | 0.054 |
| 47 | 0.59 | 0.76 | 0.61 | 0.138 | 0.053 |
| 48 | 0.86 | 0.63 | 0.418 | 0.108 | 0.043 |
| 49 | 2.83 | 2.86 | 0.143 | 0.054 | 0.043 |
| 50 | 0.23 | 0.16 | 0.047 | 0.046 | 0.047 |
| 51 | 0.23 | 0.16 | 0.052 | 0.048 | 0.042 |
| 52 | 0.61 | 0.77 | 0.499 | 0.137 | 0.053 |
| 53 | 18.8 | 11.4 | 0.887 | 0.18 | 0.059 |
| 54 | 0.58 | 0.75 | 0.471 | 0.128 | 0.05 |
| 55 | 0.47 | 0.57 | 0.331 | 0.086 | 0.047 |
| 56 | 0.25 | 0.17 | 0.043 | 0.042 | 0.043 |
| 57 | 0.26 | 0.18 | 0.041 | 0.041 | 0.04 |
| 58 | 0.53 | 0.23 | 0.033 | 0.043 | 0.039 |

TABLE 3
Cell binding and ELISA screening against CD30L

| Clone | Cell Binding | ELISA Binding |
|---|---|---|
| ID | HuCD30L Flow (MFI) | HuCD30L ELISA (OD) |
| 59 | 9785.31 | 0.06 |
| 60 | 1090.51 | 0.23 |
| 61 | 361.75 | 1.42 |

TABLE 4
ELISA screening against CD30L and CD30:CD30L blocking data

| | Elisa Binding (OD) | | | Elisa CD30 Neutralization | | | |
|---|---|---|---|---|---|---|---|
| | cyCD30L | cyCD30L | cyCD30L | (% inhibition) | | | |
| | (1/50 | (1/500 | (1/5000 | huCD30L | huCD30L | huCD30L | huCD30L |
| Clone ID | dil) | dil) | dil) | (undil) | (1/10 dil) | (1/100 dil) | (1/1000 dil) |
| 1 | 2.833 | 2.866 | 1.155 | 98% | 98% | 34% | 0% |
| 2 | 3.762 | 3.177 | 1.226 | 98% | 98% | 45% | 1% |
| 3 | 3.199 | 2.348 | 0.552 | 98% | 95% | 17% | 3% |
| 4 | 2.575 | 2.378 | 0.583 | 98% | 96% | 10% | 0% |
| 5 | 2.899 | 2.774 | 0.974 | 98% | 98% | 36% | 7% |
| 6 | 3.482 | 2.574 | 0.793 | 98% | 98% | 25% | 0% |
| 7 | 3.665 | 2.742 | 0.837 | 98% | 98% | 21% | 0% |
| 8 | 3.443 | 2.266 | 0.616 | 98% | 98% | 25% | 0% |
| 9 | 3.432 | 2.539 | 0.794 | 98% | 98% | 29% | 0% |
| 10 | 3.356 | 2.254 | 0.655 | 98% | 98% | 28% | 0% |
| 11 | 2.99 | 2.507 | 0.675 | 98% | 97% | 28% | 0% |
| 12 | 2.442 | 2.636 | 0.69 | 98% | 97% | 16% | 0% |
| 13 | 2.787 | 2.522 | 0.699 | 98% | 97% | 17% | 3% |
| 14 | 3.228 | 2.08 | 0.557 | 98% | 97% | 17% | 0% |
| 15 | 2.644 | 2.48 | 0.656 | 98% | 97% | 22% | 7% |
| 16 | 3.227 | 2.006 | 0.582 | 98% | 97% | 23% | 6% |
| 17 | 2.689 | 2.636 | 0.69 | 98% | 97% | 17% | 0% |
| 18 | 2.567 | 2.559 | 0.637 | 98% | 96% | 20% | 4% |
| 19 | 3.328 | 1.905 | 0.453 | 98% | 96% | 8% | 0% |
| 20 | 3.349 | 2.583 | 0.679 | 98% | 98% | 24% | 6% |
| 21 | 3.372 | 2.008 | 0.511 | 98% | 96% | 16% | 1% |
| 22 | 2.752 | 2.441 | 0.631 | 98% | 96% | 24% | 10% |
| 23 | 2.845 | 2.474 | 0.625 | 98% | 96% | 17% | 3% |
| 24 | 2.583 | 2.416 | 0.617 | 98% | 96% | 17% | 6% |
| 25 | 2.537 | 2.283 | 0.534 | 98% | 95% | 9% | 0% |
| 26 | 2.303 | 2.267 | 0.574 | 98% | 95% | 18% | 5% |
| 27 | 2.693 | 2.293 | 0.503 | 98% | 94% | 16% | 4% |
| 28 | 2.589 | 2.244 | 0.516 | 98% | 96% | 15% | 2% |
| 29 | 2.85 | 2.287 | 0.522 | 98% | 92% | 18% | 8% |
| 30 | 2.694 | 2.314 | 0.578 | 98% | 96% | 12% | 0% |
| 31 | 2.473 | 2.269 | 0.493 | 98% | 88% | 9% | 1% |
| 32 | 2.461 | 2.37 | 0.507 | 98% | 89% | 16% | 1% |
| 33 | 2.727 | 2.154 | 0.473 | 98% | 91% | 9% | 4% |
| 34 | 3.249 | 1.683 | 0.366 | 98% | 93% | 12% | 2% |
| 35 | 2.703 | 2.218 | 0.484 | 98% | 92% | 19% | 5% |
| 36 | 2.428 | 2.288 | 0.525 | 98% | 94% | 10% | 0% |
| 37 | 3.065 | 1.631 | 0.353 | 99% | 86% | 10% | 4% |
| 38 | 2.694 | 2.352 | 0.568 | 98% | 92% | 16% | 5% |
| 39 | 3.336 | 1.72 | 0.358 | 98% | 93% | 13% | 1% |
| 40 | 1.192 | 0.682 | 0.198 | 0% | 0% | 0% | 0% |
| 41 | 2.063 | 1.382 | 0.409 | 64% | 52% | 13% | 18% |
| 42 | 2.633 | 1.893 | 0.36 | 98% | 76% | 11% | 6% |
| 43 | 3.064 | 1.385 | 0.278 | 98% | 76% | 8% | 0% |
| 44 | 2.911 | 1.185 | 0.223 | 98% | 66% | 5% | 0% |
| 45 | 1.138 | 0.248 | 0.067 | 1% | 0% | 0% | 0% |
| 46 | 1.388 | 0.338 | 0.079 | 67% | 57% | 26% | 5% |
| 47 | 1.785 | 0.447 | 0.094 | 0% | 0% | 28% | 0% |
| 48 | 1.163 | 0.279 | 0.071 | 9% | 5% | 0% | 0% |
| 49 | 0.064 | 0.058 | 0.045 | 0% | 0% | 0% | 2% |
| 50 | 0.046 | 0.048 | 0.063 | 3% | 4% | 2% | 0% |
| 51 | 0.052 | 0.049 | 0.043 | 1% | 0% | 4% | 1% |
| 52 | 1.713 | 0.463 | 0.092 | 5% | 0% | 0% | 0% |
| 53 | 1.84 | 0.397 | 0.089 | 83% | 7% | 0% | 3% |
| 54 | 1.716 | 0.426 | 0.102 | 0% | 22% | 0% | 0% |
| 55 | 1.253 | 0.262 | 0.069 | 7% | 3% | 9% | 14% |
| 56 | 0.057 | 0.045 | 0.049 | 0% | 0% | 0% | 0% |
| 57 | 0.046 | 0.043 | 0.046 | 0% | 0% | 0% | 1% |
| 58 | 0.052 | 0.043 | 0.043 | 0% | 0% | 0% | 0% |

### 5.2 Example 2: Functional Activity of CD30L Antibodies

When engaged by CD30L, expressed at the cell surface or in a recombinant soluble form, CD30+Karpas 299 (K299) (cells from Dr Abraham Karpas at the University of Cambridge) cells release IL-8 in the supernatant. Interleukin 8 is a chemokine produced by macrophages and other cell types such as epithelial cells, and functions in activating an inflammatory and/or immune response. A monoclonal antibody binding to CD30L in such a way to neutralize CD30-CD30L interaction can therefore be identified using this functional assay by detecting inhibition of IL-8 release. A dual cell, IL-8 release assay was developed with the K299 cells expressing human CD30 and B16-huCD30L or HEK-cyCD30L. Clones 1-2 effectively reduced, inhibited, and/or prevented IL-8 release. In this assay, the anti-CD30L antibody test articles showed marked improvements (e.g. lower IC50) over the control test article anti-CD30L Reference 1 antibody (Ref1, comprising heavy chain of SEQ ID NO: 768 and light chain of SEQ ID NO: 769), an antibody that binds CD30L and blocks its interaction with CD30. Table 5 shows the results of the assay.

TABLE 5
IL-8 release, dual cell assay

| | Inhibition of IL-8 release in a | | Inhibition of IL-8 release in a | |
|---|---|---|---|---|
| | dual cell assay (B16-huCD30L | | dual cell assay (HEK293- | |
| | cells + K299 cells) | | cyCD30L cells + K299 cells) | |
| | IC₅₀ (nM) | | IC₅₀ (nM) | |
| Clone | Mouse IgG | Human IgG1 | Mouse IgG | Human IgG1 |
| Ref1 | | 1.74 | | 0.68 |
| 1 | 1.99 | 1.1 | 0.55 | 0.22 |
| 2 | 1.20 | 0.6 | 0.46 | 0.24 |

### 5.3 Example 3: Anti-CD30L Antibody Binding to Cell-Expressed CD30L

Anti-CD30L antibody binding to a CD30L target was determined by flow cytometry, using B16 cells transfected to express human CD30L (compared to non-transfected B16), and using HEK293 cells transfected to express cynomolgus CD30L (compared to non-transfected HEK293). In brief, the cell lines were detached with a non-enzymatic EDTA-based reagent, incubated on ice with increasing concentrations of anti-CD30L antibodies (from 0.002 nM to 33.5 nM) in flow cytometry buffer, and washed to remove unbound molecules. Cell surface bound antibodies were detected using a fluorescently labeled anti-human-IgG or anti-mouse IgG, depending on the Fc portion of the antibodies. After incubation with the secondary antibody, the cells were washed, fixed in a paraformaldehyde solution, and analyzed by flow cytometry to detect antibody binding. The results from this assay are shown in Table 6.

TABLE 6
Summary of cell-based binding and blocking assays

| | Binding of | | Antibody mediated blocking of | |
|---|---|---|---|---|
| | antibodies to B16- | | huCD30 binding to B16- | |
| | huCD30L cells | | huCD30L cells | |
| | EC₅₀ (nM) | | IC₅₀ (nM) | |
| Clone | Mouse IgG | Human IgG1 | Mouse IgG | Human IgG1 |
| Ref1 | | 1.74 | | 2.77 |
| 1 | 1.95 | 1.41 | 3.06 | 1.35 |
| 2 | 1.21 | 0.81 | 1.84 | 0.86 |

To assess the potency of the disclosed anti-CD30L antibodies to inhibit binding between CD30 and CD30L, a flow cytometry-based assay was developed using cells expressing human CD30L (B16-huCD30L) and cells expressing cyno CD30L (HEK-cyCD30L). In this assay, the binding of soluble fluorescently-labeled CD30-Fc to CD30 was detected at the surface of the cells by flow cytometry. The pre-incubation of these cells with increasing concentrations of blocking anti-CD30L antibodies inhibited the binding of fluorescent CD30-Fc to the cell surface in a dose dependent manner. The results from this assay are shown in Table 6. In both assays (binding and blocking), the anti-CD30L antibody test articles show improvements (e.g. lower EC50 and lower IC50 values) over the control test article anti-CD30L Reference 1 antibody (Ref1, comprising heavy chain of SEQ ID NO: 768 and light chain of SEQ ID NO: 769), an antibody that binds CD30L and blocks its interaction with CD30.

### 5.4 Example 4: Anti-CD30L Antibody Binding to Human Primary Cells

Peripheral blood mononuclear cells (PBMCs) were isolated from freshly collected whole blood from Ulcerative Colitis (UC) and Crohn's Disease (CD) patients, by conventional density gradient centrifugation. To induce CD30L expression on primary lymphocytes, the isolated cells were stimulated overnight with Phorbol 12-myristate 13-acetate (PMA) and ionomycin. The next day, the stimulated cells, along with non-stimulated cells kept as control, were collected, washed and incubated on ice with increasing concentrations of fluorescently labeled anti-CD30L antibodies or isotype control (from 0.001 nM to 60 nM). After washing to remove unbound antibodies, the cells were fixed in a paraformaldehyde solution and analyzed by flow cytometry to quantify cell surface antibody binding. Typical results from this assay are shown in Table 7.

TABLE 7
Binding to primary lymphocytes from UC and
CD patients stimulated with PMA/ionomycin.

| | Patient 1 | Patient 2 | Patient 3 | Patient 4 |
|---|---|---|---|---|
| | (CD, | (UC, | (UC, | (CD, |
| | #04-021) | #03-041) | #02-180) | #01-051) |
| Antibody | Binding | Binding | Binding | Binding |
| Clone | EC₅₀ (nM) | EC₅₀ (nM) | EC₅₀ (nM) | EC₅₀ (nM) |
| Ref1 (HC of | 3.63 | 3.12 | 3.07 | 2.82 |
| SEQ ID NO: | | | | |
| 768 & LC of | | | | |
| SEQ ID NO: | | | | |
| 769) | | | | |
| 1 | 7.45 | 5.43 | 5.57 | 4.53 |
| 2 | 2.40 | 1.55 | 1.71 | 1.72 |

### 5.5 Example 5: Generation and Identification of Additional Anti-CD30L Antibodies

To identify additional antibody sequences, including additional CDR sequences, that can specifically bind to CD30L, block CD30-CD30L interaction, and block CD30L-mediated CD30 signaling, multiple additional antibody campaigns were independently performed. Three different and independently constructed and humanized mouse strains (#1, #2, or #3) were used for antibody generation. Two of the mouse strains (#2 or #3) were designed to maximize antibody diversity. All mice were inoculated with human CD30L expressing cells (B16 cell line transfected to express human CD30L), DNA encoding human CD30L, and recombinant human CD30L protein. At certain time points following the start of the immunizations, the serum of the mice was screened for reactivity to human CD30L, cynomolgus CD30L and mouse CD30L. B cells from mice with strong titers were imported to a BEACON instrument for single B cell screening. The BEACON screening aimed to identify CD30L blocking antibodies that exhibited human and cynomolgus species cross reactivity and on-cell target binding.

B cells from the BEACON screening were exported to multi-well plates for VH and VL sequencing and production of recombinant antibodies. Purified recombinant antibodies were tested for binding to recombinant human CD30L, cynomolgus CD30L and mouse CD30L and to cells expressing human and cynomolgus CD30L (Table 8). In addition, the ability of each antibody to block CD30L binding to CD30 was tested. Briefly, individual anti-human biosensors were loaded with purified IgG (normalized to 10 μg/mL) and a baseline was measured for each biosensor. Biosensors were saturated with biotin and with polyclonal huIgG and a baseline was measured. Loaded biosensors were then allowed to bind CD30L and the association rate was measured for the binding. Biosensors were moved to buffer wells to measure the dissociation rate. Biosensors were reloaded with CD30L and moved to wells with CD30. Association of CD30 results in an increase in relative units over background (0.15) and is indicative of a non-blocking antibody clone.

TABLE 8
Purified recombinant antibodies bind CD30L and compete with CD30

| | | rhCD30L | rcyCD30L | rmCD30L | hCD30L | Parental | rhCD30L: |
|---|---|---|---|---|---|---|---|
| Clone | Mouse | (+) | (+) | (+) | B16 (+) | (−) | rhCD30 |
| ID | Strain | ELISA | ELISA | ELISA | FLOW | FLOW | Blocking |
| 62 | #1 | 0.555 | 0.332 | 0.047 | 4826784 | 11596 | 0.0557 |
| 86 | #1 | 1.435 | 0.748 | 0.045 | 4493586 | 11572 | 0.0631 |
| 87 | #1 | 0.051 | 0.057 | 0.045 | 10958 | 10992 | No |
| | | | | | | | binding |
| 88 | #1 | 0.283 | 0.109 | 0.045 | 1052602 | 10999 | 0.0547 |
| 89 | #1 | 0.449 | 0.146 | 0.058 | 1984901 | 11095 | 0.0532 |
| 90 | #1 | 1.641 | 0.798 | 0.046 | 5997151 | 11468 | 0.068 |
| 63 | #1 | 1.871 | 0.724 | 0.046 | 6070933 | 11113 | 0.0592 |
| 91 | #1 | 0.268 | 0.104 | 0.044 | 1051940 | 10786 | 0.0547 |
| 92 | #1 | 0.251 | 0.057 | 0.045 | 47034 | 10869 | 0.0642 |
| 93 | #1 | 0.144 | 0.066 | 0.045 | 234134 | 11002 | No |
| | | | | | | | binding |
| 94 | #1 | 0.982 | 0.245 | 0.059 | 4535943 | 11254 | 0.0551 |
| 95 | #1 | 0.866 | 0.262 | 0.045 | 1503954 | 11012 | 0.053 |
| 96 | #1 | 0.048 | 0.057 | 0.046 | 12183 | 11785 | No |
| | | | | | | | binding |
| 97 | #1 | 0.05 | 0.055 | 0.055 | 13077 | 11219 | No |
| | | | | | | | binding |
| 64 | #1 | 2.041 | 0.951 | 0.049 | 5667300 | 11683 | 0.0633 |
| 98 | #1 | 0.049 | 0.056 | 0.048 | 35192 | 11365 | No |
| | | | | | | | binding |
| 99 | #1 | 0.048 | 0.055 | 0.049 | 11119 | 11472 | No |
| | | | | | | | binding |
| 65 | #1 | 1.966 | 1.378 | 0.047 | 6913949 | 11367 | 0.0522 |
| 100 | #1 | 0.049 | 0.055 | 0.048 | 10750 | 11549 | No |
| | | | | | | | binding |
| 66 | #1 | 2.047 | 0.056 | 0.045 | 19034 | 11297 | 0.2575 |
| 101 | #1 | 0.052 | 0.055 | 0.046 | 10757 | 11622 | No |
| | | | | | | | binding |
| 67 | #1 | 2.352 | 0.089 | 0.046 | 197523 | 11267 | 0.2053 |
| 68 | #2 or #3 | 1.707 | 0.867 | 0.044 | 5578433 | 10925 | 0.055 |
| 102 | #2 or #3 | 0.412 | 0.231 | 0.045 | 2417049 | 11384 | 0.0697 |
| 69 | #2 or #3 | 0.353 | 0.138 | 0.047 | 4929180 | 11328 | 0.2273 |
| 103 | #2 or #3 | 0.05 | 0.058 | 0.049 | 11442 | 10952 | No |
| | | | | | | | binding |
| 70 | #2 or #3 | 1.514 | 0.709 | 0.046 | 6946359 | 12003 | 0.0687 |
| 104 | #2 or #3 | 0.238 | 0.065 | 0.047 | 774533 | 11524 | 0.0394 |
| 71 | #2 or #3 | 1.733 | 0.855 | 0.046 | 5382299 | 11676 | 0.0529 |
| 72 | #2 or #3 | 0.813 | 0.262 | 0.046 | 5242171 | 11521 | 0.0696 |
| 73 | #2 or #3 | 0.634 | 0.169 | 0.045 | 4377595 | 11469 | 0.0646 |
| 74 | #2 or #3 | 1.561 | 0.634 | 0.047 | 6843276 | 12458 | 0.0807 |
| 105 | #2 or #3 | 0.048 | 0.058 | 0.048 | 10970 | 11632 | No |
| | | | | | | | binding |
| 106 | #2 or #3 | 0.051 | 0.056 | 0.046 | 11924 | 11608 | No |
| | | | | | | | binding |
| 107 | #2 or #3 | 0.049 | 0.057 | 0.05 | 10499 | 11162 | No |
| | | | | | | | binding |
| 108 | #2 or #3 | 0.048 | 0.081 | 0.051 | 13882 | 15347 | No |
| | | | | | | | binding |
| 75 | #2 or #3 | 1.177 | 0.349 | 0.044 | 5907158 | 11251 | 0.0739 |
| 76 | #1 | 1.665 | 0.494 | 0.045 | 5656499 | 11271 | 0.0684 |
| 77 | #1 | 1.74 | 0.96 | 0.05 | 5186725 | 11680 | 0.0926 |
| 78 | #1 | 1.545 | 0.972 | 0.049 | 6565681 | 11228 | 0.0959 |
| 79 | #1 | 1.464 | 0.538 | 0.05 | 6388847 | 11415 | 0.0718 |
| 109 | #1 | 1.762 | 0.703 | 0.055 | 6506456 | 10919 | 0.0749 |
| 80 | #1 | 0.941 | 0.475 | 0.048 | 8301813 | 12058 | 0.0635 |
| 110 | #1 | 1.952 | 0.999 | 0.051 | 6507107 | 11209 | 0.1115 |
| 81 | #1 | 1.596 | 1.412 | 0.052 | 7156876 | 11429 | 0.2136 |
| 82 | #1 | 1.408 | 0.982 | 0.048 | 8422953 | 11194 | 0.071 |
| 83 | #1 | 1.855 | 1.214 | 0.049 | 6828103 | 11641 | 0.0855 |
| 84 | #1 | 1.643 | 0.732 | 0.052 | 6810066 | 12542 | 0.0638 |
| 85 | #1 | 1.03 | 0.283 | 0.067 | 5284859 | 12331 | 0.08 |

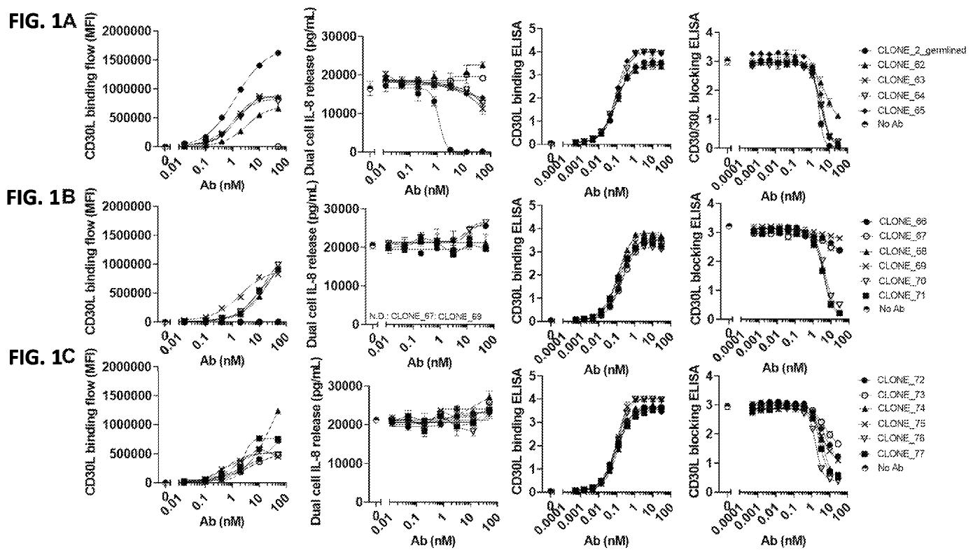

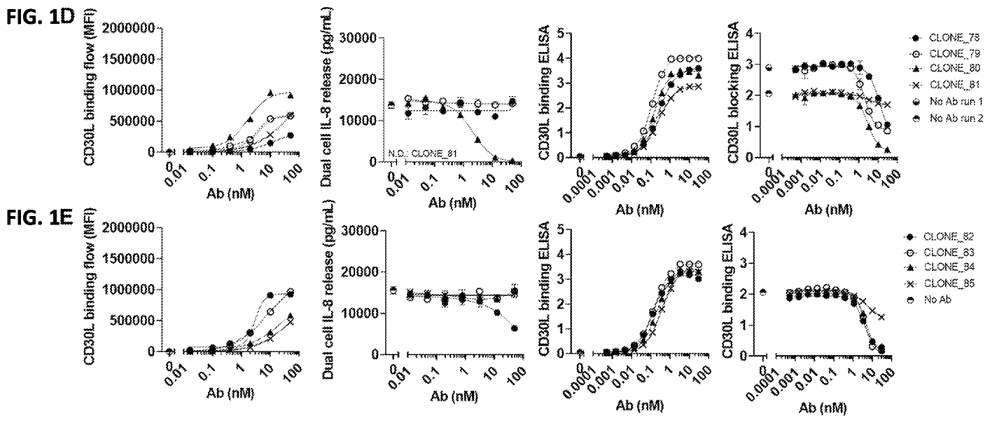

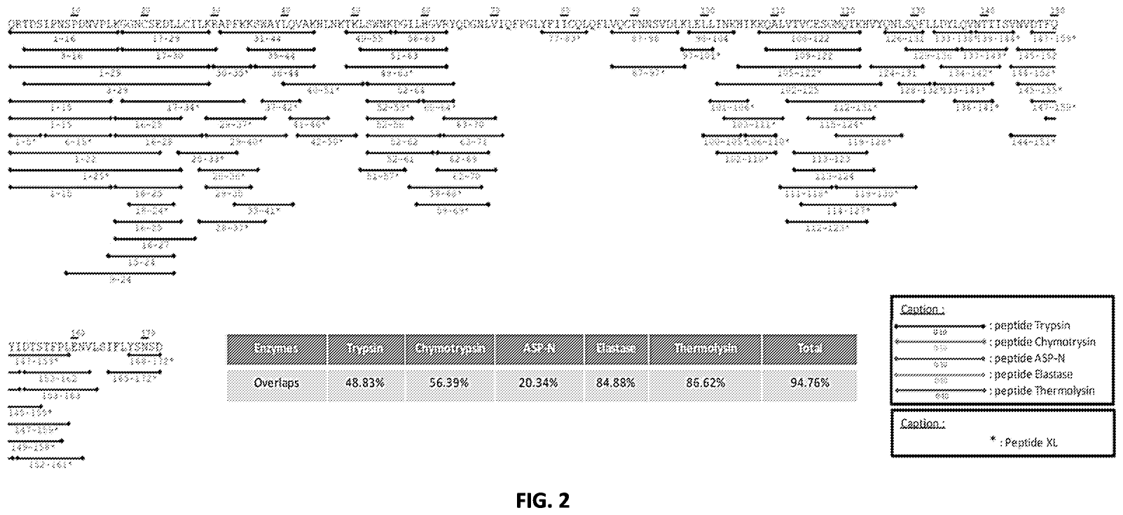

A subset of antibody clones (clones 62-85) with robust CD30L binding and CD30 blocking activity were selected for larger scale production based on the binding and blocking data, as well as VH and VL sequence considerations. Affinities to human CD30L were determined using a Carterra instrument (Table 9). Recombinant blocking activity of the clones were also evaluated in an ELISA format, in which CD30L was added to the plate, followed by CD30 and a dose response of the antibody clones. The IC50 of the clones determined in such ELISA assay was shown in Table 9. Clones were also tested in a functional assay. Briefly, when engaged by CD30L, expressed at the cell surface or in a recombinant soluble form, CD30+ Karpas 299 (K299) cells release IL-8 in the supernatant. Interleukin 8 is a chemokine produced by macrophages and other cell types such as epithelial cells, and functions in activating an inflammatory and/or immune response. A monoclonal antibody binding to CD30L in such a way to neutralize CD30-CD30L interaction can therefore be identified using this functional assay by detecting inhibition of IL-8 release. A dual cell, IL-8 release assay was developed with the K299 cells endogenously expressing human CD30 and B16-huCD30L (“dual cell assay”). The ability of each tested clone to inhibit or block CD30L-mediated CD30 signaling was shown in in Table 9, in which “++” indicates complete blocking of IL-8 release in the dual cell assay, “+” indicates inhibition of IL-8 release in the dual cell assay, and “−” indicates no inhibition of IL-8 release in the dual cell assay despite of inhibiting or blocking recombinant CD30-CD30L binding. FIG. 1A-1E shows a dose response of CD30L antibodies in dual cell IL-8 release assay, huCD30L B16 flow assay and binding/blocking ELISA which were the basis for Table 9.

TABLE 9
Characterization of select anti-CD30L antibodies

| | | Recombinant | Dual |
|---|---|---|---|
| Clone | KD | blocking ELISA | cell |
| ID | (Carterra) | (IC50 nM) | assay |
| 62 | 8.77E−09 | 5.936 | − |
| 63 | 1.85E−09 | 3.178 | + |
| 64 | 1.57E−09 | 3.174 | + |
| 65 | 1.52E−09 | 3.66 | + |
| 66 | 4.23E−10 | 12.25 | − |
| 67 | 1.15E−09 | 12.02 | − |
| 68 | 1.59E−08 | 3.658 | − |
| 69 | 4.83E−09 | 3.195 | NA |
| 70 | 2.83E−10 | 4.192 | − |
| 71 | 2.07E−08 | 3.576 | − |
| 72 | 4.72E−09 | 4.045 | − |
| 73 | 7.41E−09 | 4.791 | − |
| 74 | 3.58E−10 | 3.712 | − |
| 75 | 3.19E−09 | 3.394 | − |
| 76 | 5.57E−10 | 1.669 | − |
| 77 | 4.16E−10 | 2.716 | − |
| 78 | 3.13E−10 | 11.76 | − |
| 79 | 8.57E−10 | 2.023 | NA |
| 80 | 3.54E−09 | 2.739 | ++ |
| 81 | 1.37E−07 | 2.763 | NA |
| 82 | 5.59E−10 | 3.508 | + |
| 83 | 1.94E−10 | 3.742 | − |
| 84 | 3.23E−10 | 4.604 | − |
| 85 | 1.22E−08 | 6.375 | − |

Taking the above Examples (Examples 1-5) together, at least three different humanized mouse strains have been immunized, multiple independent antibody campaigns have been conducted at different time points, and at least 110 antibodies have been generated and tested. Of the 110 anti-CD30L antibody clones tested, the vast majority of the 110 clones bound to CD30L and the vast majority of the 110 clones blocked CD30-CD30L recombinant protein binding (see Tables 2-9). Among the 110 clones, seven clones (clones 1, 2, 63-65, 80 and 82) blocked or inhibited CD30L-mediated CD30 signaling, e.g. CD30L-mediated IL-8 release (see Tables 5, 6, and 9).

### 5.6 Example 6: Structural Determinants of Binding to CD30L and Blocking/Inhibiting CD30L-Mediated CD30 Signaling

As shown above, 110 clones across multiple antibody campaigns with at least three different mouse strains were generated, with the vast majority of which demonstrating robust binding to recombinant CD30L and cell surface CD30L and blocking/inhibiting recombinant CD30-CD30L binding. Despite such large number of clones from diverse campaigns and background, 7 anti-CD30L (clones 1, 2, 63-65, 80 and 82) both blocked/inhibited CD30-CD30L recombination protein interaction and functionally blocked/inhibited CD30L-mediated CD30 signaling in a dual cell assay. Therefore, the structural (including the primary structure, e.g. amino acid sequence) features of the 7 clones that confer them the binding and functional blocking capability were determined. The clones were sequenced and the CDR sequences of the 7 clones were identified according to Kabat, Chothia, AbM, Contact, IMGT, and Aho numbering scheme. Each of the 6 CDRs (CDR-H1, CDR-H2, CDR-H3, CDR-L1, CDR-L2, and CDR-L3) was aligned to determine the sequence and sequence features common to all of them, which can be the determinants of their common capacity to both block/inhibit CD30-CD30L recombination protein interaction and functionally block/inhibit CD30L-mediated CD30 signaling in a dual cell assay. As shown in Tables 10-16, despite coming from different mice and different antibody campaign, the corresponding CDR sequences of the 7 clones aligned well with identical residues at certain positions and conserved variations at others, suggesting that these conserved sequence features confer their common capacity to both block/inhibit CD30-CD30L recombination protein interaction and functionally block/inhibit CD30L-mediated CD30 signaling in a dual cell assay.

Accordingly, consensus sequences for each of the 6 CDRs (CDR-H1, CDR-H2, CDR-H3, CDR-L1, CDR-L2, and CDR-L3) was established based on the clones as shown in Tables 10-16 to capture the common sequence features, which can be the determinants of their common capacity to both block/inhibit CD30-CD30L recombination protein interaction and functionally block/inhibit CD30L-mediated CD30 signaling in a dual cell assay. In each of Tables 10-16, the consensus_1 sequence shows the common sequence features of clones 1, 2, and 80, which completely blocked both CD30-CD30L recombination protein interaction and CD30L-mediated CD30 signaling in a dual cell assay; the consensus_2 sequence shows the common sequence features of clones 1, 2, 63-65, 80 and 82, which both block/inhibit CD30-CD30L recombination protein interaction and functionally block/inhibit CD30L-mediated CD30 signaling in a dual cell assay

TABLE 10
Alignment and Consensus Sequences of Clones 1, 2, 63-65, 80 and 82 based on Kabat numbering (where applicable, space was
inserted into the CDR sequences to demonstrate the alignment; in the table ″++″ indicates complete blocking of IL-8 release in the dual
cell assay, ″+″ indicates inhibition of IL-8 release in the dual cell assay as shown in FIG. 1)

| Clone ID/ | | | | | | | Functional |
|---|---|---|---|---|---|---|---|
| Consensus | CDR-H1 | CDR-H2 | CDR-H3 | CDR-L1 | CDR-L2 | CDR-L3 | blocking |
| 1 | GYYWS (SEQ | YI FYSGYTKYNPSLKS | DR W D F DY | RASQGIRNNLG (SEQ | AESSLQS (SEQ | LQDFNYPYT (SEQ | ++ |
| (germlined) | ID NO: 107) | (SEQ ID NO: 147) | (SEQ ID NO: 187) | ID NO: 307) | ID NO: 347) | ID NO: 387) | |
| 2 | DYAMH (SEQ | GI S WRSGNIGYADSVKG | DKGIGFNWNYEGF DY | RASQGISNHLA (SEQ | AASSLQS (SEQ | QQYVTYPLT (SEQ | ++ |
| (germlined) | ID NO: 117) | (SEQ ID NO: 157) | (SEQ ID NO: 197) | ID NO: 317) | ID NO: 357) | ID NO: 397) | |
| 80 | SYDMH (SEQ | SI G I GGDTYYPGSVKG | GEWDLLWY FMDV | RASQGISSWLA (SEQ | AASSLQS (SEQ | QQANSFPLT (SEQ | ++ |
| | ID NO: 482) | (SEQ ID NO: 520) | (SEQ ID NO: 545) | ID NO: 570) | ID NO: 595) | ID NO: 620) | |
| 63 | SYSMN (SEQ | YI S SSSSTIYDADSVKG | EAYPGYYYN YMDV | RASQSISSRLA (SEQ | KASNLES (SEQ | QQYNSYSRT (SEQ | + |
| | ID NO: 467) | (SEQ ID NO: 492) | (SEQ ID NO: 530) | ID NO: 555) | ID NO: 580) | ID NO: 605) | |
| 64 | NYAMT (SEQ | AI S GFGGSTYYADSVKG | D HDYYA F DY | RASQSISSYLN (SEQ | AVSSLQS (SEQ | QQSYSTPYT (SEQ | + |
| | ID NO: 472) | (SEQ ID NO: 497) | (SEQ ID NO: 535) | ID NO: 560) | ID NO: 585) | ID NO: 610) | |
| 65 | NYAMT (SEQ | AI S GYGGSTYYAASVKG | D HDYYA F DY | RASQSINSYLN (SEQ | SASSLQS (SEQ | QQSYSTPYT (SEQ | + |
| | ID NO: 477) | (SEQ ID NO: 515) | (SEQ ID NO: 540) | ID NO: 565) | ID NO: 590) | ID NO: 615) | |
| 82 | NAWMS (SEQ | RIKSKTYGGTTDYAAPVKG | DPWNYVNYNY FMDV | RASQDIRNYLA (SEQ | AASTLQS (SEQ | QNYFSVPLT (SEQ | + |
| | ID NO: 487) | (SEQ ID NO: 525) | (SEQ ID NO: 550) | ID NO: 575) | ID NO: 600) | ID NO: 625) | |
| Consensus_1 | X1-Y-X2-X3-X4, | X1-I-X2-X3-X4-X5-G-X6- | X1-X2-X3-X4-X5-X6- | R-A-S-Q-G-I-X1-X2- | A-X1-S-S-L-Q-S, | X1-Q-X2-X3-X4- | |
| based on | wherein X1 is G, | X7-X8-Y-X9-X10-S-X11- | X7-W-X8-X9-X10-X11- | X3-L-X4, | wherein X1 is A | X5-P-X6-T, | |
| Kabat | D, or S; X2 is | K-X12, wherein X1 is | F-X12-D-X13, | wherein X1 is R | or E (SEQ ID | wherein X1 is L | |
| | Y, A, or D; X3 | Y, G, or S; X2 is S, | wherein X1 is D or | or S; X2 is N or | NO: 752) | or Q; X2 is D, | |
| | is W or M; and | G, or none; X3 is F, | none; X2 is R, K, | S; X3 is N, H | | Y, or A; X3 is | |
| | X4 is S or H | W, or I; X4 is Y, R, | or G; X3 is G, E, | or W; and X4 is | | F, V, or N; X4 | |
| | (SEQ ID NO: | or none; X5 is G or S; | or none; X4 is I, | G or A | | is N, T, or S; | |
| | 712) | X6 is Y, N, or D; X7 | W, or none; X5 is | (SEQ ID NO: 744) | | X5 is Y or F; | |
| | | is T or I; X8 is K, G, | G, D, or none; X6 | | | X6 is Y or L | |
| | | or Y; X9 is N, A, or | is F, L, or none; | | | (SEQ ID | |
| | | P; and X10 is P, D, or | X7 is N, L, or none; | | | NO: 760) | |
| | | G; X11 is L or V; and | X8 is N, Y, or none; | | | | |
| | | X12 is S or G (SEQ ID | X9 is Y, or none; | | | | |
| | | NO: 724) | X10 is D, E, or | | | | |
| | | | none; X11 is G or | | | | |
| | | | none; X12 is M or | | | | |
| | | | none; and X13 is Y | | | | |
| | | | or V (SEQ ID NO: | | | | |
| | | | 736) | | | | |
| Consensus_2 | X1-X2-X3-X4-X5, | X1-I-X2-X3-X4-X5-X6- | X1-X2-X3-X4-X5-X6- | R-A-S-Q-X1-I-X2- | X1-X2-S-X3-L- | X1-Q-X2-X3-X4- | |
| based on | wherein X1 is G, | X7-X8-X9-X10-X11-X12- | X7-X8-X9-X10-X11- | X3-X4-L-X5, | X4-S, wherein | X5-X6-X7-T, | |
| Kabat | D, N, or S; X2 | X13-X14-K-X15, wherein | X12-X13-X14-D-X15, | wherein X1 is G, | X1 is A, K, or | wherein X1 is L | |
| | is Y or A; X3 is | X1 is Y, G, S, A or R; | wherein X1 is D or | S, or D; X2 is | S; X2 is A, E, | or Q; X2 is D, | |
| | Y, A, S, W, or | X2 is S, G, or none; | none; X2 is R, K, | R, S, or N; X3 is | or V; X3 is S, | Y, A, or S; X3 | |
| | D; X4 is W or | X3 is F, W, I, S, G | G, P, or none; X3 | N or S; X4 is N, | N, or T; and X4 | is F, V, N, or | |
| | M; and X5 | or T; X4 is Y, R, S, | is G, E, W, or | H, W, R, or Y; | is Q or E | Y; X4 is N, T, | |
| | is N, T, S or H | F, or none; X5 is G or | none; X4 is I, W, | and X5 is G, A, | (SEQ ID NO: | or S; X5 is Y, | |
| | (SEQ ID NO: | S; X6 is G or S; X7 is | A, N, or none; X5 | or N (SEQ ID | 753) | F, T, or V; X6 | |
| | 713) | Y, N, D, T or S; X8 is | is G, D, Y, or | NO: 745) | | is P or S; X7 | |
| | | T or I; X9 is K, G, Y, | none; X6 is F, L, | | | is Y, L or R | |
| | | or D; X10 is Y or D; | P, H, V, or none; | | | (SEQ ID NO: 761) | |
| | | X11 is N, A, or P; and | X7 is N, L, G, D, | | | | |
| | | X12 is P, D, G, or A; | or none; X8 is W or | | | | |
| | | X13 is S or P; X14 is | Y; X9 is N, Y, or | | | | |
| | | L or V; and X15 is S | none; X10 is Y, A, | | | | |
| | | or G (SEQ ID NO: 725) | or none; X11 is D, | | | | |
| | | | E, N, or none; X12 | | | | |
| | | | is G or none; X13 | | | | |
| | | | is F or Y; X14 is | | | | |
| | | | M ornone; and X15 | | | | |
| | | | is Y or V | | | | |
| | | | (SEQ ID NO: 737) | | | | |

TABLE 11
Alignment and Consensus Sequences of Clones 1, 2, 63-65, 80 and 82 based on Chothia numbering (where applicable, space was
inserted into the CDR sequences to demonstrate the alignment; in the table ″++″ indicates complete blocking of IL-8 release in the dual
cell assay, ″+″ indicates inhibition of IL-8 release in the dual cell assay as shown in FIG. 1)

| Clone ID/ | | | | | | | Functional |
|---|---|---|---|---|---|---|---|
| Consensus | CDR-H1 | CDR-H2 | CDR-H3 | CDR-L1 | CDR-L2 | CDR-L3 | blocking |
| 1 | GGSISGY (SEQ | FYSGY (SEQ | DR W D F DY | RASQGIRNNLG (SEQ | AESSLQS (SEQ | LQDFNYPYT (SEQ | ++ |
| (germlined) | ID NO: 105) | ID NO: 145) | (SEQ ID NO: 185) | ID NO: 305) | ID NO: 345) | ID NO: 385) | |
| 2 | GFNFDDY (SEQ | S WRSGN (SEQ | DKGIGFNWNYEGF DY | RASQGISNHLA (SEQ | AASSLQS (SEQ | QQYVTYPLT (SEQ | ++ |
| (germlined) | ID NO: 115) | ID NO: 155) | (SEQ ID NO: 195) | ID NO: 315) | ID NO: 355) | ID NO: 395) | |
| 80 | GFTFSSY (SEQ | G I GGD (SEQ | GEWDLLWY FMDV | RASQGISSWLA (SEQ | AASSLQS (SEQ | QQANSFPLT (SEQ | ++ |
| | ID NO: 480) | ID NO: 518) | (SEQ ID NO: 543) | ID NO: 568) | ID NO: 593) | ID NO: 618) | |
| 63 | GFMFSSY (SEQ | S SSSST (SEQ | EAYPGYYYN YMDV | RASQSISSRLA (SEQ | KASNLES (SEQ | QQYNSYSRT (SEQ | + |
| | ID NO: 465) | ID NO: 490) | (SEQ ID NO: 528) | ID NO: 553) | ID NO: 578) | ID NO: 603) | |
| 64 | GFTFSNY (SEQ | S GFGGS (SEQ | D HDYYA F DY | RASQSISSYLN (SEQ | AVSSLQS (SEQ | QQSYSTPYT (SEQ | + |
| | ID NO: 470) | ID NO: 495) | (SEQ ID NO: 533) | ID NO: 558) | ID NO: 583) | ID NO: 608) | |
| 65 | GFTFSNY (SEQ | S GYGGS (SEQ | D HDYYA F DY | RASQSINSYLN (SEQ | SASSLQS (SEQ | QQSYSTPYT (SEQ | + |
| | ID NO: 475) | ID NO: 513) | (SEQ ID NO: 538) | ID NO: 563) | ID NO: 588) | ID NO: 613) | |
| 82 | GITFSNA (SEQ | KSKTYGGT (SEQ | DPWNYVNYNY FMDV | RASQDIRNYLA (SEQ | AASTLQS (SEQ | QNYFSVPLT (SEQ | + |
| | ID NO: 485) | ID NO: 523) | (SEQ ID NO: 548) | ID NO: 573) | ID NO: 598) | ID NO: 623) | |
| Consensus_1 | G-X1-X2-X3-X4- | X1-X2-X3-X4-G- | X1-X2-X3-X4-X5-X6-X7- | R-A-S-Q-G-I-X1-X2- | A-X1-S-S-L-Q-S, | X1-Q-X2-X3-X4-X5- | |
| based on | X5-Y, wherein X1 | X5, wherein X1 | W-X8-X9-X10-X11-F- | X3-L-X4, wherein | wherein X1 is | P-X6-T, wherein X1 | |
| Chothia | is G or F; X2 | is S, G, or | X12-D-X13, wherein X1 | X1 is R or S; X2 | A or E (SEQ ID | is L or Q; X2 is | |
| | is S, N, or T; | none; X2 is F, | is D or none; X2 is | is N or S; X3 is | NO: 752) | D, Y, or A; X3 is | |
| | X3 is I or F; X4 | W, or I; X3 is | R, K, or G; X3 is G, | N, H or W; and X4 | | F, V, or N; X4 is | |
| | is S or D; and | Y, R, or none; | E, or none; X4 is I, | is G or A | | N, T, or S; X5 is | |
| | X5 is G, D, or | X4 is S or G; | W, or none; X5 is G, | (SEQ ID NO: 744) | | Y or F; X6 is | |
| | S (SEQ ID NO: | and X5 is Y, N, | D, or none; X6 is F, | | | Y or L (SEQ ID | |
| | 714) | or D (SEQ ID | L, or none; X7 is N, | | | NO: 760) | |
| | | NO: 726) | L, or none; X8 is N, | | | | |
| | | | Y, or none; X9 is | | | | |
| | | | Y, or none; X10 is | | | | |
| | | | D, E, or none; X11 is | | | | |
| | | | G or none; X12 is M | | | | |
| | | | or none; and X13 is | | | | |
| | | | Y or V (SEQ ID NO: | | | | |
| | | | 736) | | | | |
| Consensus_2 | G-X1-X2-X3-X4- | X1-X2-X3-X4-X5- | X1-X2-X3-X4-X5-X6-X7- | R-A-S-Q-X1-I-X2- | X1-X2-S-X3-L- | X1-Q-X2-X3-X4-X5- | |
| based on | X5-X6, wherein | X6, wherein X1 | X8-X9-X10-X11-X12- | X3-X4-L-X5, | X4-S, wherein | X6-X7-T, wherein | |
| Chothia | X1 is G, F or I; | is S, G, or | X13-X14-D-X15, | wherein X1 is G, | X1 is A, K, | X1 is L or Q; X2 | |
| | X2 is S, N, T, | none; X2 is F, | wherein X1 is D or | S, or D; X2 is R, | or S; X2 is A, | is D, Y, A, or S; | |
| | or M; X3 is I or | W, G, S, I, or | none; X2 is R, K, G, | S, or N; X3 is N | E, or V; X3 is | X3 is F, V, N, or | |
| | F; X4 is S or | T; X3 is Y, R, | P, or none; X3 is G, | or S; X4 is N, H, | S, N, or T; and | Y; X4 is N, T, or | |
| | D; X5 is G, D, | S, F, or none; | E, W, or none; X4 | W, R, or Y; and X5 | X4 is Q or E | S; X5 is Y, F, T, | |
| | S, or N; and X6 | X4 is S or G; | is I, W, A, N, or | is G, A, or N (SEQ | (SEQ ID NO: | or V; X6 is P or | |
| | is Y or A | X5 is G or S; | none; X5 is G, D, Y, | ID NO: 745) | 753) | S; X7 is Y, L or | |
| | (SEQ ID NO: 715) | and X6 is Y, N, | ornone; X6 is F, L, | | | R (SEQ ID NO: 761) | |
| | | D, T, or S | P, H, V, or none; X7 | | | | |
| | | (SEQ ID NO: 727) | is N, L, G, D, or | | | | |
| | | | none; X8 is W or Y; | | | | |
| | | | X9 is N, Y, or none; | | | | |
| | | | X10 is Y, A, or none; | | | | |
| | | | X11 is D, E, N, or | | | | |
| | | | none; X12 is G or | | | | |
| | | | none; X13 is F or Y; | | | | |
| | | | X14 is M or none; and | | | | |
| | | | X15 is Y or V | | | | |
| | | | (SEQ ID NO: 737) | | | | |

TABLE 12
Alignment and Consensus Sequences of Clones 1, 2, 63-65, 80 and 82 based on AbM numbering (where applicable, space was
inserted into the CDR sequences to demonstrate the alignment; in the table ″++″ indicates complete blocking of IL-8 release in the dual
cell assay, ″+″ indicates inhibition of IL-8 release in the dual cell assay as shown in FIG. 1)

| Clone ID/ | | | | | | | Functional |
|---|---|---|---|---|---|---|---|
| Consensus | CDR-H1 | CDR-H2 | CDR-H3 | CDR-L1 | CDR-L2 | CDR-L3 | blocking |
| 1 | GGSISGYYWS (SEQ | YI FYSGYTK | DR W D F DY | RASQGIRNNLG (SEQ | AESSLQS (SEQ | LQDFNYPYT (SEQ | ++ |
| (germlined) | ID NO: 106) | (SEQ ID NO: 146) | (SEQ ID NO: 186) | ID NO: 306) | ID NO: 346) | ID NO: 386) | |
| 2 | GFNFDDYAMH (SEQ | GI S WRSGNIG | DKGIGFNWNYEGF DY | RASQGISNHLA (SEQ | AASSLQS (SEQ | QQYVTYPLT (SEQ | ++ |
| (germlined) | ID NO: 116) | (SEQ ID NO: 156) | (SEQ ID NO: 196) | ID NO: 316) | ID NO: 356) | ID NO: 396) | |
| 80 | GFTFSSYDMH (SEQ | SI G I GGDTY | GEWDLLWY FMDV | RASQGISSWLA (SEQ | AASSLQS (SEQ | QQANSFPLT (SEQ | ++ |
| | ID NO: 481) | (SEQ ID NO: 519) | (SEQ ID NO: 544) | ID NO: 569) | ID NO: 594) | ID NO: 619) | |
| 63 | GFMFSSYSMN (SEQ | YI S SSSSTIY | EAYPGYYYN YMDV | RASQSISSRLA (SEQ | KASNLES (SEQ | QQYNSYSRT (SEQ | + |
| | ID NO: 466) | (SEQ ID NO: 491) | (SEQ ID NO: 529) | ID NO: 554) | ID NO: 579) | ID NO: 604) | |
| 64 | GFTFSNYAMT (SEQ | AI S GFGGSTY | D HDYYA F DY | RASQSISSYLN (SEQ | AVSSLQS (SEQ | QQSYSTPYT (SEQ | + |
| | ID NO: 471) | (SEQ ID NO: 496) | (SEQ ID NO: 534) | ID NO: 559) | ID NO: 584) | ID NO: 609) | |
| 65 | GFTFSNYAMT (SEQ | AI S GYGGSTY | D HDYYA F DY | RASQSINSYLN (SEQ | SASSLQS (SEQ | QQSYSTPYT (SEQ | |
| | ID NO: 476) | (SEQ ID NO: 514) | (SEQ ID NO: 539) | ID NO: 564) | ID NO: 589) | ID NO: 614) + | |
| 82 | GITFSNAWMS (SEQ | RIKSKTYGGTTD | DPWNYVNYNY FMDV | RASQDIRNYLA (SEQ | AASTLQS (SEQ | QNYFSVPLT (SEQ | |
| | ID NO: 486) | (SEQ ID NO: 524) | (SEQ ID NO: 549) | ID NO: 574) | ID NO: 599) | ID NO: 624) + | |
| Consensus_1 | G-X1-X2-X3-X4- | X1-I-X2-X3-X4-X5-G- | X1-X2-X3-X4-X5-X6- | R-A-S-Q-G-I-X1-X2- | A-X1-S-S-L-Q-S, | X1-Q-X2-X3-X4- | |
| based on | X5-Y-X6-X7-X8, | X6-X7-X8, wherein | X7-W-X8-X9-X10-X11- | X3-L-X4, wherein | wherein X1 is | X5-P-X6-T, | |
| AbM | wherein X1 is G | X1 is Y, G, or S; | F-X12-D-X13, wherein | X1 is R or S; X2 | A or E | wherein X1 is L | |
| | or F; X2 is S, | X2 is S, G, or none; | X1 is D or none; X2 | is N or S; X3 is | (SEQ ID NO: | or Q; X2 is D, | |
| | N, or T; X3 is I | X3 is F, W, or I; | is R, K, or G; X3 is | N, H or W; and X4 | 752) | Y, or A; X3 is | |
| | or F; X4 is S or | X4 is Y, R, or | G, E, or none; X4 is | is G or A | | F, V, or N; X4 | |
| | D; X5 is G, D, | none; X5 is G or S; | I, W, or none; X5 is | (SEQ ID NO: 744) | | is N, T, or S; | |
| | or S; X6 is Y, A, | X6 is Y, N, or D; | G, D, or none; X6 is | | | X5 is Y or F; | |
| | or D; X7 is W or | X7 is T or I; X8 | F, L, or none; X7 is | | | X6 is Y or L | |
| | M; and X8 is S | is K, G, or Y | N, L, or none; X8 is | | | (SEQ ID NO: 760) | |
| | or H | (SEQ ID NO: 728) | N, Y, or none; X9 is | | | | |
| | (SEQ ID NO: 716) | | Y, or none; X10 is | | | | |
| | | | D, E, or none; X11 | | | | |
| | | | is G or none; X12 is | | | | |
| | | | M or none; and X13 | | | | |
| | | | is Y or V (SEQ ID | | | | |
| | | | NO: 736) | | | | |
| Consensus_2 | G-X1-X2-X3-X4- | X1-I-X2-X3-X4-X5- | X1-X2-X3-X4-X5-X6- | R-A-S-Q-X1-I-X2- | X1-X2-S-X3-L- | X1-Q-X2-X3-X4- | |
| based on | X5-X6-X7-X8-X9, | X6-X7-X8-X9, | X7-X8-X9-X10-X11- | X3-X4-L-X5, | X4-S, wherein | X5-X6-X7-T, | |
| AbM | wherein X1 is G, | wherein X1 is Y, G, | X12-X13-X14-D-X15, | wherein X1 is G, | X1 is A, K, or | wherein X1 is L | |
| | F or I; X2 is S, | S, A or R; X2 is S, | wherein X1 is D or | S, or D; X2 is R, | S; X2 is A, | or Q; X2 is D, | |
| | N, T, or M; X3 | G, or none; X3 is | none; X2 is R, K, G, | S, or N; X3 is N | E, or V; X3 is | Y, A, or S; X3 | |
| | is I or F; X4 is | F, W, I, S, G or T; | P, or none; X3 is G, | or S; X4 is N, H, | S, N, or T; | is F, V, N, or | |
| | S or D; X5 is G, | X4 is Y, R, S, F, | E, W, or none; X4 is | W, R, or Y; and X5 | and X4 is Q or | Y; X4 is N, T, | |
| | D, S, or N; X6 is | or none; X5 is G or | I, W, A, N, or none; | is G, A, or N | E (SEQ ID NO: | or S; X5 is Y, | |
| | Y or A; X7 is Y, | S; X6 is G or S; X7 | X5 is G, D, Y, or | (SEQ ID NO: 745) | 753) | F, T, or V; X6 | |
| | A, D, S or W; X8 | is Y, N, D, T or S; | none; X6 is F, L, P, | | | is P or S; X7 is | |
| | is W or M; and | X8 is T or I; X9 is | H, V, or none; X7 is | | | Y, L or R | |
| | X9 is S, H, N, | K, G, Y, or D (SEQ | N, L, G, D, or none; | | | (SEQ ID NO: 761) | |
| | or T (SEQ ID NO: | ID NO: 729) | X8 is W or Y; X9 is | | | | |
| | 717) | | N, Y, or none; X10 | | | | |
| | | | is Y, A, or none; | | | | |
| | | | X11 is D, E, N, or | | | | |
| | | | none; X12 is G or | | | | |
| | | | none; X13 is F or Y; | | | | |
| | | | X14 is M or none; | | | | |
| | | | and X15 is Y or V | | | | |
| | | | (SEQ ID NO: 737) | | | | |

TABLE 13
Alignment and Consensus Sequences of Clones 1, 2, 63-65, 80 and 82 based on Contact numbering (where applicable, space was
inserted into the CDR sequences to demonstrate the alignment; in the table ″++″ indicates complete blocking of IL-8 release in the dual
cell assay, ″+″ indicates inhibition of IL-8 release in the dual cell assay as shown in FIG. 1)

| Clone ID/ | | | | | | | Functional |
|---|---|---|---|---|---|---|---|
| Consensus | CDR-H1 | CDR-H2 | CDR-H3 | CDR-L1 | CDR-L2 | CDR-L3 | blocking |
| 1 | SGYYWS (SEQ | WIGYI FYSGYTK | ARDR W D F D | RNNLGWY (SEQ | LLIYAESSLQ (SEQ | LQDFNYPY (SEQ | ++ |
| (germlined) | ID NO: 108) | (SEQ ID NO: 148) | (SEQ ID NO: 188) | ID NO: 308) | ID NO: 348) | ID NO: 388) | |
| 2 | DDYAM (SEQ | WVSGI S WRSGNIG | AKDKGIGFNWNYEGF D | SNHLAWF (SEQ | SLIYAASSLQ (SEQ | QQYVTYPL (SEQ | ++ |
| (germlined) | ID NO: 118) | (SEQ ID NO: 158) | (SEQ ID NO: 198) | ID NO: 318) | ID NO: 358) | ID NO: 398) | |
| 80 | SSYDMH (SEQ | WVSSI G I GGDTY | AR GEWDLLWY FMD | SSWLAWY (SEQ | LLIYAASSLQ (SEQ | QQANSFPL (SEQ | ++ |
| | ID NO: 483) | (SEQ ID NO: 521) | (SEQ ID NO: 546) | ID NO: 571) | ID NO: 596) | ID NO: 621) | |
| 63 | SSYSMN (SEQ | WVSYI S SSSSTIY | TR EAYPGYYYN YMD | SSRLAWY (SEQ | LLIYKASNLE (SEQ | QQYNSYSR (SEQ | + |
| | ID NO: 468) | (SEQ ID NO: 493) | (SEQ ID NO: 531) | ID NO: 556) | ID NO: 581) | ID NO: 606) | |
| 64 | SNYAMT (SEQ | WVSAI S GFGGSTY | AKD HDYYA F D | SSYLNWY (SEQ | LLIYAVSSLQ (SEQ | QQSYSTPY (SEQ | + |
| | ID NO: 473) | (SEQ ID NO: 498) | (SEQ ID NO: 536) | ID NO: 561) | ID NO: 586) | ID NO: 611) | |
| 65 | SNYAMT (SEQ | WVSAI S GYGGSTY | AKD HDYYA F D | NSYLNWY (SEQ | LLIYSASSLQ (SEQ | QQSYSTPY (SEQ | + |
| | ID NO: 478) | (SEQ ID NO: 516) | (SEQ ID NO: 541) | ID NO: 566) | ID NO: 591) | ID NO: 616) | |
| 82 | SNAWMS (SEQ | WVGRIKSKTYGGTTD | TTDPWNYVNYNY FMD | RNYLAWY (SEQ | LLVYAASTLQ (SEQ | QNYFSVPL (SEQ | + |
| | ID NO: 488) | (SEQ ID NO: 526) | (SEQ ID NO: 551) | ID NO: 576) | ID NO: 601) | ID NO: 626) | |
| Consensus_1 | X1-X2-Y-X3- | W-X1-X2-X3-I-X4-X5- | A-X1-X2-X3-X4-X5-X6- | X1-X2-X3-L- | X1-L-I-Y-A-X2-S-S- | X1-Q-X2-X3-X4-X5- | |
| based on | X4-X5, | X6-X7-G-X8-X9-X10, | X7-X8-W-X9-X10-X11- | X4-W-X5, | L-Q wherein X1 is | P-X6, wherein X1 is | |
| Contact | wherein X1 | wherein X1 is I or | X12-F-X13-D, wherein | wherein X1 is | L or S; and X2 is | Lor Q; X2 is D, Y, | |
| | is S or D; | V; X2 is G or S; | X1 is K or R; X2 is | R or S; X2 is | A or E (SEQ ID | or A; X3 is F, V, or | |
| | X2 is G, D, | X3 is Y, G, or S; | D or none; X3 is R, | N or S; X3 | NO: 754) | N; X4 is N, T, or S; | |
| | or S; X3 is | X4 is S, G, or | K, or G; X4 is G, E, | is N, H or W; | | X5 is Y or F; X6 is | |
| | Y, A, or D; | none; X5 is F, W, | or none; X5 is I, W, | X4 is G or A; | | Y or L (SEQ ID NO: | |
| | X4 is W or M; | or I; X6 is Y, R, | or none; X6 is G, D, | and X5 is Y or | | 762) | |
| | and X5 is S | or none; X7 is G | or none; X7 is F, L, | F (SEQ | | | |
| | or H (SEQ ID | or S; X8 is Y, N, | or none; X8 is N, L, | ID NO: 746) | | | |
| | NO: 718) | or D; X9 is T or I; | or none; X9 is N, Y, | | | | |
| | | X10 is K, G, or Y | or none; X10 is Y, | | | | |
| | | (SEQ IDN O: 730) | or none; X11 is D, | | | | |
| | | | E, or none; X12 is | | | | |
| | | | G or none; and X13 | | | | |
| | | | is M or none | | | | |
| | | | (SEQ ID NO: 738) | | | | |
| Consensus_2 | X1-X2-X3-X4- | W-X1-X2-X3-I-X4-X5- | X1-X2-X3-X4-X5-X6- | X1-X2-X3-L-X4- | X1-L-X2-Y-X3-X4-S- | X1-Q-X2-X3-X4-X5- | |
| based on | X5-X6, | X6-X7-X8-X9-X10- | X7-X8-X9-X10-X11- | W-X5, | X5-L-X6, | X6-X7, wherein X1 | |
| Contact | wherein X1 is | X11, wherein X1 is | X12-X13-X14-X15-X16- | wherein X1 is | wherein X1 is L | is L or Q; X2 is D, | |
| | S or D; X2 | I or V; X2 is G or | D, wherein X1 is A | R, S, or N; | or S; X2 is I or | Y, A, or S; X3 is | |
| | is G, D, N, | S; X3 is Y, G, S, | or T; X2 is K, R, or | X2 is N or S; | V; X3 is A, K, or | F, V, N, or Y; X4 is | |
| | or S; X3 is | A or R; X4 is S, | T; X3 is D or none; | X3 is N, H, W, | S; X4 is A, E, or | N, T, or S; X5 is | |
| | Y or A; X4 | G, or none; X5 is | X4 is R, K, G, P, or | R, or Y; X4 is | V; X5 is S, N, or | Y, F, T, or V; X6 | |
| | is Y, A, S, | F, W, I, S, G or T; | none; X5 is G, E, W, | G, A, or N; | T; and X6 is Q or | is P or S; X7 is Y, | |
| | W, or D; X5 | X6 is Y, R, S, F, | or none; X6 is I, W, | and X5 Y or F | E (SEQ ID NO: 755) | L or R (SEQ | |
| | is W or M; | or none; X7 is G | A, N, or none; X7 | (SEQ ID NO: | | ID NO: 763) | |
| | and X6 is N, | or S; X8 is G or | is G, D, Y, or none; | 747) | | | |
| | T, S or H | S; X9 is Y, N, D, | X8 is F, L, P, H, V, | | | | |
| | (SEQ ID | T or S; X10 is T | or none; X9 is N, | | | | |
| | NO: 719) | or I; X 10 is K, | L, G, D, or none; | | | | |
| | | G, Y, or D (SEQ | X10 is W or Y; X11 | | | | |
| | | ID NO: 731) | is N, Y, or none; | | | | |
| | | | X12 is Y, A, or none; | | | | |
| | | | X13 is D, E, N, or | | | | |
| | | | none; X14 is G or | | | | |
| | | | none; X15 is F or | | | | |
| | | | Y; and X16 is M or | | | | |
| | | | none (SEQ ID | | | | |
| | | | NO: 739) | | | | |

TABLE 14
Alignment and Consensus Sequences of Clones 1, 2, 63-65, 80 and 82 based on IMGT numbering (where applicable, space was
inserted into the CDR sequences to demonstrate the alignment; in the table ″++″ indicates complete blocking of IL-8 release in the dual
cell assay, ″+″ indicates inhibition of IL-8 release in the dual cell assay as shown in FIG. 1)

| Clone ID/ | | | | | | | Functional |
|---|---|---|---|---|---|---|---|
| Consensus | CDR-H1 | CDR-H2 | CDR-H3 | CDR-L1 | CDR-L2 | CDR-L3 | blocking |
| 1 | GGSISGYY (SEQ | I FYSGYT (SEQ | ARDR W D F DY | QGIRNN (SEQ | AE (SEQ ID | LQDFNYPYT (SEQ | ++ |
| (germlined) | ID NO: 109) | ID NO: 149) | (SEQ ID NO: 189) | ID NO: 309) | NO: 349) | ID NO: 389) | |
| 2 | GFNFDDYA (SEQ | I S WRSGNI (SEQ | AKDKGIGFNWNYEGF DY | QGISNH (SEQ | AA (SEQ ID | QQYVTYPLT (SEQ | |
| (germlined) | ID NO: 119) | ID NO: 159) | (SEQ ID NO: 199) | ID NO: 319) | NO: 359) | ID NO: 399) | ++ |
| 80 | GFTFSSYD (SEQ | I G I GGDT (SEQ | AR GEWDLLWY FMDV | QGISSW (SEQ | AA (SEQ ID | QQANSFPLT (SEQ | |
| | ID NO: 484) | ID NO: 522) | (SEQ ID NO: 547) | ID NO: 572) | NO: 597) | ID NO: 622) | ++ |
| 63 | GFMFSSYS (SEQ | I S SSSSTI (SEQ | TR EAYPGYYYN YMDV | QSISSR (SEQ | KA (SEQ ID | QQYNSYSRT (SEQ | + |
| | ID NO: 469) | ID NO: 494) | (SEQ ID NO: 532) | ID NO: 557) | NO: 582) | ID NO: 607) | |
| 64 | GFTFSNYA (SEQ | I S GFGGST (SEQ | AKD HDYYA F DY | QSISSY (SEQ | AV (SEQ ID | QQSYSTPYT (SEQ | + |
| | ID NO: 474) | ID NO: 499) | (SEQ ID NO: 537) | ID NO: 562) | NO: 587) | ID NO: 612) | |
| 65 | GFTFSNYA (SEQ | I S GYGGST (SEQ | AKD HDYYA F DY | QSINSY (SEQ | SA (SEQ ID | QQSYSTPYT (SEQ | + |
| | ID NO: 479) | ID NO: 517) | (SEQ ID NO: 542) | ID NO: 567) | NO: 592) | ID NO: 617) | |
| 82 | GITFSNAW (SEQ | IKSKTYGGTT (SEQ | TTDPWNYVNYNY FMDV | QDIRNY (SEQ | AA (SEQ ID | QNYFSVPLT (SEQ | + |
| | ID NO: 489) | ID NO: 527) | SEQ ID NO: 552) | ID NO: 577) | NO: 602) | ID NO: 627) | |
| Consensus_1 | G-X1-X2-X3-X4- | I-X1-X2-X3-X4-G- | A-X1-X2-X3-X4-X5-X6- | Q-G-I-X1-X2-X3, | A-X1, wherein | X1-Q-X2-X3-X4-X5-P- | |
| based on | X5-Y-X6, | X5-X6, wherein | X7-X8-W-X9-X10-X11- | wherein X1 is R | X1 is A or E | X6-T, wherein X1 is L | |
| IMGT | wherein X1 is | X1 is S, G, or | X12-F-X13-D-X14, | or S; X2 is N or | (SEQ ID NO: | or Q; X2 is D, Y, or A; | |
| | G or F; X2 is | none; X2 is F, W, | wherein X1 is K or | S; and X3 is N, | 756) | X3 is F, V, or N; X4 is | |
| | S, N, or T; X3 | or I; X3 is Y, R, | R; X2 is D or none; | H or W (SEQ ID | | N, T, or S; X5 is Y or | |
| | is I or F; X4 | or none; X4 is G | X3 is R, K, or G; X4 | NO: 748) | | F; X6 is Y or L (SEQ ID | |
| | is S or D; X5 | or S; X5 is Y, N, | is G, E, or none; | | | NO: 760) | |
| | is G, D, or S; | or D; and X6 is | X5 is I, W, or none; | | | | |
| | and X6 is Y, A, | T or I | X6 is G, D, or none; | | | | |
| | or D (SEQ ID | (SEQ ID NO: 732) | X7 is F, L, or | | | | |
| | NO: 720) | | none; X8 is N, L, or | | | | |
| | | | none; X9 is N, Y, or | | | | |
| | | | none; X10 is Y, or | | | | |
| | | | none; X11 is D, E, | | | | |
| | | | or none; X12 is G or | | | | |
| | | | none; X13 is M or | | | | |
| | | | none; and X14 is Y | | | | |
| | | | or V (SEQ ID | | | | |
| | | | NO: 740) | | | | |
| Consensus_2 | G-X1-X2-X3-X4- | I-X1-X2-X3-X4-X5- | X1-X2-X3-X4-X5-X6- | Q-X1-I-X2-X3-X4, | X1-X2, wherein | X1-Q-X2-X3-X4-X5- | |
| based on | X5-X6-X7, | X6-X7, wherein | X7-X8-X9-X10-X11- | wherein X1 is G, | X1 is A, K, | X6-X7-T, wherein X1 | |
| IMGT | wherein X1 is | X1 is S, G, or | X12-X13-X14-X15-X16- | S, or D; X2 is | or S; and X2 | is Lor Q; X2 is D, Y, A, | |
| | G, For I; X2 is | none; X2 is F, W, | D-X17, wherein X1 | R, S, or N; X3 | is A, E, or V | or S; X3 is F, V, N, or | |
| | S, N, T, or M; | I, S, G or T; X3 | is A or T; X2 is K, | is N or S; and | (SEQ ID | Y; X4 is N, T, or S; X5 | |
| | X3 is I or F; | is Y, R, S, F, or | R, or T; X3 is D or | X4 is N, H, W, | NO: 757) | is Y, F, T, or V; X6 is | |
| | X4 is S or D; | none; X4 is G or | none; X4 is R, K, G, | R, or Y | | P or S; X7 is Y, L or R | |
| | X5 is G, D, S, | S; X5 is G or S; | P, or none; X5 is G, | (SEQ ID NO: 749) | | (SEQ ID NO: 761) | |
| | or N; X6 is Y | X6 is Y, N, D, T | E, W, or none; X6 is | | | | |
| | or A; and X7 is | or S; and X7 is | I, W, A, N, or | | | | |
| | Y, A, D, S, or | T or I (SEQ ID | none; X7 is G, D, Y, | | | | |
| | W | NO: 733) | or none; X8 is F, L, | | | | |
| | (SEQ ID NO: 721) | | P, H, V, or none; X9 | | | | |
| | | | is N, L, G, D, or | | | | |
| | | | none; X10 is W or Y; | | | | |
| | | | X11 is N, Y, or | | | | |
| | | | none; X12 is Y, A, | | | | |
| | | | or none; X13 is D, | | | | |
| | | | E, N, or none; X14 | | | | |
| | | | is G or none; X15 is | | | | |
| | | | F or Y; X16 is M or | | | | |
| | | | none; and X17 is Y | | | | |
| | | | or V (SEQ ID NO: 741) | | | | |

TABLE 15
Alignment and Consensus Sequences of Clones 1, 2, 63-65, 80 and 82 based on Aho numbering (where applicable, space was
inserted into the CDR sequences to demonstrate the alignment; in the table ″++″ indicates complete blocking of IL-8 release in the dual
cell assay, ″+″ indicates inhibition of IL-8 release in the dual cell assay as shown in FIG. 1)

| Clone ID/ | | | | | | | Functional |
|---|---|---|---|---|---|---|---|
| Consensus | CDR-H1 | CDR-H2 | CDR-H3 | CDR-L1 | CDR-L2 | CDR-L3 | blocking |
| 1 | VSGGSISGYY (SEQ | I FYSGYTKYNPSLKSR | DR W DFD | ASQGIRNN (SEQ | AESSLQSGVPSR | DFNYPY (SEQ | ++ |
| (germlined) | ID NO: 628) | (SEQ ID NO: 642) | (SEQ ID NO: 656) | ID NO: 670) | (SEQ ID NO: 684) | ID NO: 698) | |
| 2 | ASGFNFDDYA (SEQ | I S WRSGNIGYADSVKGR | DKGIGENWNYEGF D | ASQGISNH (SEQ | AASSLQSGVPSR | YVTYPL (SEQ | ++ |
| (germlined) | ID NO: 629) | (SEQ ID NO: 643) | (SEQ ID NO: 657) | ID NO: 671) | (SEQ ID NO: 685) | ID NO: 699) | |
| 80 | VSGFTFSSYD (SEQ | I G I GGDTYYPGSVKGR | GEWDLLWY FMD | ASQGISSW (SEQ | AASSLQSGVPSR | ANSFPL (SEQ | |
| | ID NO: 630) | (SEQ ID NO: 644) | (SEQ ID NO: 658) | ID NO: 672) | (SEQ ID NO: 686) | ID NO: 700) | ++ |
| 63 | ASGFMFSSYS (SEQ | I S SSSSTIYDADSVKGR | EAYPGYYYN YMD | ASQSISSR (SEQ | KASNLESGVPSR | YNSYSR (SEQ | |
| | ID NO: 631) | (SEQ ID NO: 645) | (SEQ ID NO: 659) | ID NO: 673) | (SEQ ID NO: 687) | ID NO: 701) | + |
| 64 | ASGFTFSNYA (SEQ | I S GFGGSTYYADSVKGR | D HDYYA F D | ASQSISSY (SEQ | AVSSLQSGVPSR | SYSTPY (SEQ | |
| | ID NO: 632) | (SEQ ID NO: 646) | (SEQ ID NO: 660) | ID NO: 674) | (SEQ ID NO: 688) | ID NO: 702) | + |
| 65 | ASGFTFSNYA (SEQ | I S GYGGSTYYAASVKGR | D HDYYA F D | ASQSINSY (SEQ | SASSLQSGVPSR | SYSTPY (SEQ | |
| | ID NO: 633) | (SEQ ID NO: 647) | (SEQ ID NO: 661) | ID NO: 675) | (SEQ ID NO: 689) | ID NO: 703) | + |
| 82 | VSGITFSNAW (SEQ | IKSKTYGGTTDYAAPVKGR | DPWNYVNYNY FMD | ASQDIRNY (SEQ | AASTLQSGVPSR | YFSVPL (SEQ | + |
| | ID NO: 634) | (SEQ ID NO: 648) | (SEQ ID NO: 662) | ID NO: 676) | (SEQ ID NO: 690) | ID NO: 704) | |
| Consensus_1 | X1-S-G-X2-X3-X4-X5- | I-X1-X2-X3-X4-G-X5-X6- | X1-X2-X3-X4-X5-X6- | A-S-Q-G-I-X1-X2- | A-X1-S-S-L-Q-S-G- | X1-X2-X3-X4-P- | |
| based on | X6-Y-X7, wherein X1 | X7-Y-X8-X9-S-X10-K- | X7-W-X8-X9-X10- | X3, wherein X1 | V-P-S-R, | X5, wherein X1 | |
| Aho | is V or A; X2 is G | X11-R, wherein X1 is | X11-F-X12-D, | is R or S; X2 | wherein X1 is A | is D, Y, or A; | |
| | or F; X3 is S, N, | S, G, or none; X2 is | wherein X1 is D | is N or S; and | or E | X2 is F, V, or | |
| | or T; X4 is I or F; | F, W, or I; X3 is Y, | or none; X2 is R, | X3 is N, H or W | (SEQ ID NO: 758) | N; X3 is N, T, | |
| | X5 is S or D; X6 | R, or none; X4 is G or | K, or G; X3 is G, | (SEQ ID | | or S; X4 is | |
| | is G, D, or S; and | S; X5 is Y, N, or D; | E, or none; X4 is | NO: 750) | | Y or F; X5 is | |
| | X7 is Y, A, or D | X6 is T or I; X7 is K, | I, W, or none; X5 | | | Y or L | |
| | (SEQ ID NO: 722) | G, or Y; X8 is N, A, | is G, D, or none; | | | (SEQ ID NO: | |
| | | or P; X9 is P, D or G; | X6 is F, L, or | | | 764) | |
| | | X10 is L or V; and X11 | none; X7 is N, L, | | | | |
| | | is G or S (SEQ ID NO: | or none; X8 is N, | | | | |
| | | 734) | Y, or none; X9 is | | | | |
| | | | Y, or none; X10 | | | | |
| | | | is D, E, or none; | | | | |
| | | | X11 is G or none; | | | | |
| | | | and X12 is M or | | | | |
| | | | none (SEQ ID NO: | | | | |
| | | | 742) | | | | |
| Consensus_2 | X1-S-G-X2-X3-X4-X5- | 1-X1-X2-X3-X4-X5-X6- | X1-X2-X3-X4-X5-X6- | A-S-Q-X1-I-X2- | X1-X2-S-X3-L-X4- | X1-X2-X3-X4- | |
| based on | X6-X7-X8, wherein | X7-X8-X9-X10-X11-X12- | X7-X8-X9-X10-X11- | X3-X4, wherein | S-G-V-P-S-R, | X5-X6, wherein | |
| Aho | X1 is V or A; X2 | X13-K-X14-R, wherein | X12-X13-X14-D, | X1 is G, S, or | wherein X1 is A, | X1 is D, Y, A, | |
| | is G, F or I; X3 | X1 is S, G, or none; | wherein X1 is D or | D; X2 is R, S, | K, or S; X2 is A, | or S; X2 is F, | |
| | is S, N, T, or M; | X2 is F, W, I, S, G or | none; X2 is R, K, | or N; X3 is N or | E, or V; X3 is | V, N, or Y; | |
| | X4 is I or F; X5 | T; X3 is Y, R, S, F, | G, P, or none; X3 | S; and X4 is N, | S, N, or T; and | X3 is N, T, or | |
| | is S or D; X6 is | or none; X4 is G or S; | is G, E, W, or | H, W, R, or Y | X4 is Q or E | S; X4 is Y, F, | |
| | G, D, S, or N; X7 | X5 is G or S; X6 is | none; X4 is I, W, | (SEQ ID NO: 751) | (SEQ ID NO: | T, or V; X5 is | |
| | is Y or A; and X8 | Y, N, D, T or S; X7 is | A, N, or none; X5 | | 759) | P or S; X6 is | |
| | is Y, A, D, S or W | T or I; X8 is K, G, Y, | is G, D, Y, or | | | Y, L or R | |
| | (SEQ ID NO: 723) | or D; X9 is Y or D; | none; X6 is F, L, | | | (SEQ ID NO: | |
| | | X10 is N, A, or P; X11 | P, H, V, or none; | | | 765) | |
| | | is P, D, G, or A; X12 | X7 is N, L, G, D, | | | | |
| | | is S or P; X13 is L or | or none; X8 is W | | | | |
| | | V; and X14 is G or S | or Y; X9 is N, Y, | | | | |
| | | (SEQ ID NO: 735) | or none; X10 is | | | | |
| | | | Y, A, or none; X11 | | | | |
| | | | is D, E, N, or | | | | |
| | | | none; X12 is G or | | | | |
| | | | none; X13 is F or | | | | |
| | | | Y; and X14 is M or | | | | |
| | | | none | | | | |
| | | | (SEQ ID NO: 743) | | | | |

TABLE 16
Alignment and Consensus Sequences of Clones 1, 2, 63-65, 80 and 82 based on cross-linking studies described in Example 7 and
Kabat and Contact numbering scheme (where applicable, space was inserted into the CDR sequences to demonstrate the alignment; in
the table ″++″ indicates complete blocking of IL-8 release in the dual cell assay, ″+″ indicates inhibition of IL-8 release in the dual cell
assay as shown in FIG. 1)

| Clone ID/ | | | | | | | Functional |
|---|---|---|---|---|---|---|---|
| Consensus | CDR-H1 | CDR-H2 | CDR-H3 | CDR-L1 | CDR-L2 | CDR-L3 | blocking |
| 1 | GYYWS (SEQ | WIGYI FYSGYTK | DR W DF DY | RASQGIRNNLG (SEQ | AESSLQS (SEQ | LQDFNYPYT (SEQ | ++ |
| (germlined) | ID NO: 635) | (SEQ ID NO: 649) | (SEQ ID NO: 663) | ID NO: 677) | ID NO: 691) | ID NO: 705) | |
| 2 | DYAMH (SEQ | WVSGI S WRSGNIG | DKGIGFNWNYEGF DY | RASQGISNHLA (SEQ | AASSLQS (SEQ | QQYVTYPLT (SEQ | ++ |
| (germlined) | ID NO: 636) | (SEQ ID NO: 650) | (SEQ ID NO: 664) | ID NO: 678) | ID NO: 692) | ID NO: 706) | |
| 80 | SYDMH (SEQ | WVSSI G I GGDTY | GEWDLLWY FMDV | RASQGISSWLA (SEQ | AASSLQS (SEQ | QQANSFPLT (SEQ | ++ |
| | ID NO: 637) | (SEQ ID NO: 651) | (SEQ ID NO: 665) | ID NO: 679) | ID NO: 693) | ID NO: 707) | |
| 63 | SYSMN (SEQ | WVSYI S SSSSTIY | EAYPGYYYN YMDV | RASQSISSRLA (SEQ | KASNLES (SEQ | QQYNSYSRT (SEQ | + |
| | ID NO: 638) | (SEQ ID NO: 652) | (SEQ ID NO: 666) | ID NO: 680) | ID NO: 694) | ID NO: 708) | |
| 64 | NYAMT (SEQ | WVSAI S GFGGSTY | D HDYYA F DY | RASQSISSYLN (SEQ | AVSSLQS (SEQ | QQSYSTPYT (SEQ | + |
| | ID NO: 639) | (SEQ ID NO: 653) | (SEQ ID NO: 667) | ID NO: 681) | ID NO: 695) | ID NO: 709) | |
| 65 | NYAMT (SEQ | WVSAI S GYGGSTY | D HDYYA F DY | RASQSINSYLN (SEQ | SASSLQS (SEQ | QQSYSTPYT (SEQ | + |
| | ID NO: 640) | (SEQ ID NO: 654) | (SEQ ID NO: 668) | ID NO: 682) | ID NO: 696) | ID NO: 710) | |
| 82 | NAWMS (SEQ | WVGRIKSKTYGGTTD | DPWNYVNYNY FMDV | RASQDIRNYLA (SEQ | AASTLQS (SEQ | QNYFSVPLT (SEQ | + |
| | ID NO: 641) | (SEQ ID NO: 655) | (SEQ ID NO: 669) | ID NO: 683) | ID NO: 697) | ID NO: 711) | |
| Consensus_1 | X1-Y-X2-X3- | W-X1-X2-X3-I-X4-X5- | X1-X2-X3-X4-X5-X6- | R-A-S-Q-G-I-X1-X2- | A-X1-S-S-L-Q-S, | X1-Q-X2-X3-X4-X5-P- | |
| based on | X4, wherein | X6-X7-G-X8-X9-X10, | X7-W-X8-X9-X10-X11- | X3-L-X4, wherein | wherein X1 is | X6-T, wherein X1 is L | |
| Cross- | X1 is G, D, | wherein X1 is I or | F-X12-D-X13, | X1 is R or S; X2 is | A or E | or Q; X2 is D, Y, or | |
| linking | or S; X2 is | V; X2 is G or S; X3 | wherein X1 is D or | N or S; X3 is N, | (SEQ ID NO: | A; X3 is F, V, or N; | |
| | Y, A, or D; | is Y, G, or S; X4 | none; X2 is R, K, | H or W; and X4 is | 752) | X4 is N, T, or S; X5 | |
| | X3 is W or | is S, G, or none; | or G; X3 is G, E, | G or A | | is Y or F; X6 is Y or | |
| | M; and X4 is | X5 is F, W, or I; | or none; X4 is I, | (SEQ ID NO: 744) | | L (SEQ ID NO: 760) | |
| | S or H (SEQ | X6 is Y, R, or | W, or none; X5 is | | | | |
| | ID NO: 712) | none; X7 is G or S; | G, D, or none; X6 | | | | |
| | | X8 is Y, N, or D; | is F, L, or none; | | | | |
| | | X9 is T or I; X | X7 is N, L, or | | | | |
| | | 10 is K, G, or Y | none; X8 is N, Y, | | | | |
| | | (SEQ ID NO: 730) | or none; X9 is Y, | | | | |
| | | | or none; X10 is | | | | |
| | | | D, E, or none; | | | | |
| | | | X11 is G or none; | | | | |
| | | | X12 is M or none; | | | | |
| | | | and X13 is Y | | | | |
| | | | or V (SEQ ID | | | | |
| | | | NO: 736) | | | | |
| Consensus_2 | X1-X2-X3-X4- | W-X1-X2-X3-I-X4-X5- | X1-X2-X3-X4-X5-X6- | R-A-S-Q-X1-I-X2-X3- | X1-X2-S-X3-L- | X1-Q-X2-X3-X4-X5- | |
| based on | X5, wherein | X6-X7-X8-X9-X10- | X7-X8-X9-X10-X11- | X4-L-X5, wherein | X4-S, wherein | X6-X7-T, wherein X1 | |
| Cross- | X1 is G, D, | X11, wherein X1 | X12-X13-X14-D-X15, | X1 is G, S, or D; | X1 is A, K, or | is L or Q; X2 is D, | |
| linking | N, or S; X2 | is I or V; X2 is | wherein X1 is D or | X2 is R, S, or N; | S; X2 is A, E, | Y, A, or S; X3 is F, | |
| | is Y or A; X3 | G or S; X3 is Y, G, | none; X2 is R, K, | X3 is N or S; X4 | or V; X3 is S, | V, N, or Y; X4 is N, | |
| | is Y, A, S, | S, A or R; X4 is S, | G, P, or none; X3 | is N, H, W, R, or | N, or T; and | T, or S; X5 is Y, F, | |
| | W, or D; X4 | G, or none; X5 is F, | is G, E, W, or none; | Y; and X5 is G, A, | X4 is Q or E | T, or V; X6 is P or | |
| | is W or M; | W, I, S, G or T; | X4 is I, W, A, N, | or N (SEQ ID NO: | (SEQ ID NO: | S; X7 is Y, L or R | |
| | and X5 is N, | X6 is Y, R, S, F, | or none; X5 is G, | 745) | 753) | (SEQ ID NO: 761) | |
| | T, S or H | or none; X7 is G or | D, Y, or none; X6 | | | | |
| | (SEQ ID NO: | S; X8 is G or S; X9 | is F, L, P, H, V, | | | | |
| | 713) | is Y, N, D, T or S; | or none; X7 is N, | | | | |
| | | X10 is T or I; X10 | L, G, D, or none; | | | | |
| | | is K, G, Y, or D | X8 is W or Y; X9 | | | | |
| | | (SEQ ID NO: 731) | is N, Y, or none; | | | | |
| | | | X10 is Y, A, or | | | | |
| | | | none; X11 is D, E, | | | | |
| | | | N, or none; X12 | | | | |
| | | | is G or none; X13 | | | | |
| | | | is F or Y; X14 is | | | | |
| | | | M or none; and X15 | | | | |
| | | | is Y or V | | | | |
| | | | (SEQ ID NO: 737) | | | | |

### 5.7 Example 7: Identification of Amino Acid Residues on CD30L and on the Anti-CD30L Antibodies that can be Cross-Linked Via a Cross-Linker

To identify the amino acid residues on CD30L and on the functional blocking anti-CD30L clones that can be crosslinked via a crosslinker (“crosslinking residues”), cross-linking, enzyme digestion and peptide mass spectrometry studies on the antibody-CD30L complex were performed (“crosslinking study”). Since the 7 clones demonstrated sequence similarity and common primary structure (sequence) features, a representative clone, germlined clone 2 comprising VH set forth in SEQ ID NO: 6, VL set forth in SEQ ID NO:8 and IgG1 constant region set forth in SEQ ID NO: 504 was used in the study. The CD30L extracellular domain (“ECD”) comprising SEQ ID NO: 34 was used. Before starting the crosslinking study, a high-mass MALDI analysis has been performed to verify the integrity and aggregation level for each sample, including germlined clone 2 and the CD30L extracellular domain comprising SEQ ID NO: 34 used for the complex formation (data not shown).

The germlined clone 2 anti-CD30L antibody and the ECD were mixed and incubated to allow the complex formation, and then cross-linked to form covalent bond for detection. By mixing a protein sample containing non-covalent interactions with a specially developed cross-linking mixture (Bich, C et al. Anal. Chem., 2010, 82 (1), pp 172-179), non-covalent complex can be specifically detected with high-sensitivity. The covalent binding generated allows the interacting species to survive the sample preparation process and the MALDI ionization. A special High-Mass detection system allows characterizing the crosslinking amino acid residues in the High-Mass range.

Each mixture prepared for the control (antibody alone or CD30L ECD alone) and for the antibody-CD30L complex (germlined clone 2 and CD30L ECD complex) was submitted to cross-linking using DSS MALDI MS analysis kit. The protein solutions (9 μl) were mixed with 1 μl of DSS Stabilizer reagent (2 mg/ml) and incubated at room temperature. After the incubation time (180 minutes) the samples were prepared for MALDI analysis for all the samples. The samples were analysed by High-Mass MALDI MS immediately after crystallization with sinapinic acid matrix (10 mg/ml) in acetonitrile/water (1:1, v/v), TFA 0.1% (DSS MALDI Kit). In control samples, MALDI TOF peaks corresponding to theoretically calculated values for antibody only or CD30L ECD only were detected (data not shown). After cross-linking, MALDI TOF peaks corresponding to the antibody-CD30L complex (germlined clone 2 and CD30L ECD complex) were detected, demonstrating the proper formation of the antibody-CD30L complex (data not shown).

Before the cross-linking studies, peptide mass fingerprint of CD30L ECD was determined. To characterize CD30L ECD, the sample was subjected to trypsin, chymotrypsin, Asp-N, elastase and thermolysin proteolysis followed by nLC-Q-Exactive MS/MS analysis. Briefly, two tubes of 10 μL of CD30L ECD (15.34 μM) were prepared. One of the two was mixed with 1 μL of DSS d0/d12 (2 mg/mL; DMF) before 180 minutes incubation time at room temperature. The other tube was not mixed with the cross-linking regent (control). Both tubes were kept 180 minutes at room temperature. After incubation, the cross-linking reaction was stopped by adding 1 μL of Ammonium Bicarbonate (20 mM final concentration) before additional 60 minutes incubation at room temperature. Then, both tubes were dried using speedvac before 8M urea resuspension (10 μL). After mixing, DTT (1 μl, 500 mM) was added to both tubes. The mixture was then incubated 60 minutes at 37° C. After incubation, iodoacetamide (1 μl, 1M) was added to each tube before additional 60 minutes incubation at room temperature, in a dark room. After incubation, 100 μl of the proteolytic buffer were added to both tubes. Trypsin buffer contains 50 mM Ambic pH 8.5, 5% acetonitrile; chymotrypsin buffer contains Tris HCl 100 mM, CaCL2 10 mM pH 7.8; the ASP-N buffer contains Phosphate buffer 50 mM pH 7.8; elastase buffer contains Tris HCl 50 mM pH 8.0 and thermolysin buffer contains Tris HCl 50 mM, CaCL2 0.5 mM pH 9.0. For trypsin proteolysis, 100 μl of the reduced/alkyled CD30L ECD were mixed with 1 μl of trypsin (Promega) with the ratio 1/100. The proteolytic mixture was incubated overnight at 37° C. For chymotrypsin proteolysis, 100 μl of the reduced/alkyled CD30L ECD were mixed with 0.5 μl of chymotrypsin (Promega) with the ratio 1/200. The proteolytic mixture was incubated overnight at 25° C. For ASP-N proteolysis, 100 μl of the reduced/alkyled CD30L ECD were mixed with 0.5 μl of ASP-N (Promega) with the ratio 1/200. The proteolytic mixture was incubated overnight at 37° C. For elastase proteolysis, 100 μl of the reduced/alkyled CD30L ECD were mixed with 1 μl of elastase (Promega) with the ratio 1/100. The proteolytic mixture was incubated overnight at 37° C. For thermolysin proteolysis, 100 μl of the reduced/alkyled TNFSF8 were mixed with 2 μl of thermolysin (Promega) with a ratio 1/50. The proteolytic mixture was incubated overnight at 70° C. After digestion formic acid 1% final was added to the solution.

After proteolysis, 1 μl of the peptide solution generated by proteolysis was loaded onto a nano-liquid chromatography system (Ultimate 3000-RSLC) and the Q-Exactive MS analysis was then performed. Based on the results obtained from such MS analyses, overlap mapping of the peptides generated from trypsin, chymotrypsin, ASP-N, elastase, and/or thermolysin proteolysis as determined and shown in FIG. 2. Combining the peptides of Trypsin, Chymotrypsin, ASP-N, Elastase and Thermolysin proteolysis, 94.76% of the CD30L ECD sequence (SEQ ID NO: 34) is covered.

To determine crosslinking residues between CD30L and the anti-CD30L binding with high resolution, the protein complexes of CD30L ECD (SEQ ID NO: 34) and anti-CD30L (clone 2 germlined) were incubated with deuterated cross-linkers and subjected to multi-enzymatic cleavage with the enzymes and procedures as described above for determining peptide mass fingerprint of CD30L ECD. After enrichment of the cross-linked peptides, the samples were analyzed by high resolution mass spectrometry (nLC-Q-Exactive MS) and the data generated were analyzed using XQuest 2.0 and Stavrox 3.6 software.

After trypsin, chymotrypsin, ASP-N, elastase, and thermolysin proteolysis of the complex between CD30L ECD and anti-CD30L with deuterated d0d12, the nLC-orbitrap MS/MS analysis detected nine cross-linked peptides between CD30L ECD (SEQ ID NO: 34) and anti-CD30L (clone 2 germlined). The sequences and positions of cross-links are presented in the Table 17.

TABLE 17
Cross-linked peptides detected between CD30L ECD (SEQ ID NO: 34) and
anti-CD30L (clone 2 germlined)

| | | | | | | Cross- | |
|---|---|---|---|---|---|---|---|
| | | | | | | linking | |
| | | | | | | position | |
| | | | Sequence | | Cross- | on | |
| | | | on CD30L | | linking | CD30L | |
| | | | ECD | | position | ECD | Identified |
| | | Sequence on | (SEQ ID | | on anti- | (SEQ ID | on |
| Sequence | Enzyme | anti-CD30L | NO: 34) | XLType | CD30L | NO: 34) | StavroX |
| ATYYCQQYVTYP- | Thermo- | 84-95 (SEQ | 1-24 | inter- | 93 (SEQ | 16 | Yes |
| QRTDSIPNSPDNVPLK | lysin | ID NO: 8) | | protein | ID NO: 8) | | |
| GGNCSEDL-a10-b16 | | | | xl | | | |
| GLEWVSGISWR- | Trypsin | 44-54 (SEQ | 17-29 | inter- | 49 (SEQ | 21 | Yes |
| GGNCSEDLLCILK-a6- | | ID NO: 6) | | protein | ID NO: 6) | | |
| b5 | | | | xl | | | |
| SLIYAASSLQSGVPSR- | Trypsin | 46-61 (SEQ | 17-29 | inter- | 52 (SEQ | 21 | Yes |
| GGNCSEDLLCILK-a7- | | ID NO: 8) | | protein | ID NO: 8) | | |
| b5 | | | | xl | | | |
| IGFNWNYEG- | Thermo- | 102-110 | 15-26 | inter- | 108 (SEQ | 21 | Yes |
| LKGGNCSEDLLC-a7-b7 | lysin | (SEQ ID NO: | | protein | ID NO: 6) | | |
| | | 6) | | xl | | | |
| SLRLSCAASGFNFDDY | Trypsin | 17-38 (SEQ | 30-34 | inter- | 35 (SEQ | 30 | Yes |
| AMHWVR-RAPFK-a19- | | ID NO: 6) | | protein | ID NO: 6) | | |
| b1 | | | | xl | | | |
| FDYWG-KHLNKTKLS- | Elastase | 111-115 | 44-52 | inter- | 113 (SEQ | 48 | Yes |
| a3-b5 | | (SEQ ID NO: | | protein | ID NO: 6) | | |
| | | 6) | | xl | | | |
| LQSGVPSR- | Thermo- | 54-61 (SEQ | 43-50 | inter- | 56 (SEQ | 48 | Yes |
| AKHLNKTK-a3-b6 | lysin | ID NO: 8) | | protein | ID NO: 8) | | |
| | | | | xl | | | |
| ISWRSGNIGYADS- | Thermo- | 51-63 (SEQ | 62-70 | inter- | 55 (SEQ | 63 | Yes |
| VRYQDGNLV-a5-b2 | lysin | ID NO: 6) | | protein | ID NO: 6) | | |
| | | | | xl | | | |
| ISWRSGNIG YADS- | Thermo- | 51-63 (SEQ | 62-70 | inter- | 55 (SEQ | 64 | Yes |
| VRYQDGNLV-a5-b3 | lysin | ID NO: 6) | | protein | ID NO: 6) | | |
| | | | | xl | | | |

Therefore, the studies with chemical cross-linking, high-mass MALDI mass spectrometry and nLC-Orbitrap mass spectrometry characterized the amino acid residues between CD30L and a representative functional blocking anti-CD30L that can be crosslinked via a chemical crosslinker in the CD30L and anti-CD30L complex (the crosslinking residues). The analyses demonstrate that such crosslinking residues between CD30L and the representative anti-CD30L (clone 2 germlined) include the amino acids the amino acids K16, S21, R30, K48, R63, and/or Y64 of SEQ ID NO: 34, as shown in Table 17 above and Table 18 below.

TABLE 18
Crosslinking Residues between CD30L and the
representative anti-CD30L (germlined clone 2)

| | CD30L SEQ ID 34 (numbering |
|---|---|
| | based on SEQ ID NO: |
| Germlined clone 2 residue | 33 in the bracket) |
| VH H35 (CDRH1) | R30 (R92) |
| VH S49 (FR-CDRH2) | S21 (S83) |
| VH S55 (CDRH2) | R63, Y64 (R125, Y126) |
| VH Y108 (CDRH3) | S21 (S83) |
| VH Y113 (CDRH3) | K48 (K110) |
| VL S52 (CDRL2) | S21 (S83) |
| VL S56 (CDRL2) | K48 (K110) |
| VL T93 (CDRL3) | K16 (K78) |

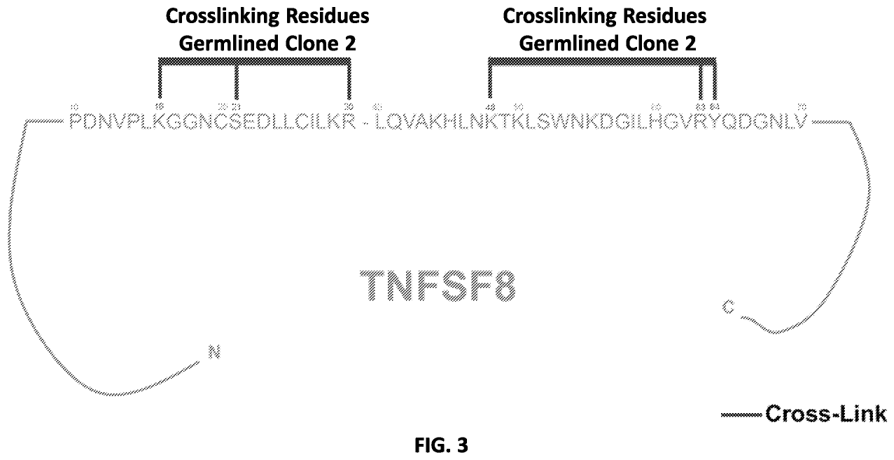

The corresponding crosslinked residues were further shown in FIG. 3. Based on the detected crosslinking residues from the anti-CD30L sequences, the CDRs from the representative anti-CD30L (germlined clone 2) were further determined by combining CDRs from Kabat numbering scheme and the Contact numbering scheme, as shown in Table 16 (referred to as based on cross-linking studies or cross-linking based). Such cross-linking based CDRs encompasses all the crosslinked residues detected in the cross-linking studies. The cross-linking based CDRs (e.g. Table 16) demonstrated robust alignment and significant similarity across the 7 function-blocking clones.

### 5.8 Example 8: Specificity of the Anti-CD30L Antibodies

Before the binding of the anti-CD30L antibodies to targets other than CD30L (off-target binding) can be assessed, an assay system was established and validated to investigate such off-target binding. Briefly, 4 representative antibodies were tested, including anti-CD30L antibody clone 1, clone 1 germlined, clone 2, and clone 2 germlined, all of which further comprising IgG1 constant regions (SEQ ID NO: 502). All 4 antibodies showed low to medium levels of background binding to untransfected HEK293 cells when tested at 2, 5 and 20 μg/mL, whilst germlined clone 2 showed low levels of background binding to untransfected HEK293 cells when tested at 2, 5 and 20 μg/mL. Binding to over-expressed TNFSF8 (CD30L), the primary target, was observed with equal intensity at all 3 concentrations (2, 5 and 20 μg/mL) tested. Based upon these data, further off-target binding was assessed at the concentration of 5 μg/mL to minimize the background.

Having established and validated the assay, all 4 antibodies (clone 1, clone 1 germlined, clone 2, and clone 2 germlined) at 5 μg/mL were screened for binding against fixed human HEK293 cells, individually expressing 5,861 full-length human plasma membrane proteins and cell surface-tethered human secreted proteins plus a further 371 human heterodimers. For the off-target screening, 5,861 expression vectors, encoding both ZsGreen1 and a full-length human plasma membrane protein or a cell surface-tethered human secreted protein, were individually arrayed in duplicate across 18 microarray slides (‘slide-sets’). In addition, vectors encoding a further 371 human heterodimers were co-arrayed across a further microarray slide. This study with a library of 6,232 (5,861+371) targets other than CD30L revealed no specific binding to 6,213 library targets, and only 19 preliminary library hits altogether for further investigation, among which is CD30L (hit #3 of the 19 hits).

To confirm the preliminary findings, each library hit was re-expressed, along with 2 control receptors, and re-tested with 5 μg/mL of each test antibody individually or control treatments. Briefly, vectors encoding each of the 19 hits identified in the library screen, plus vectors encoding CD20 and EGFR, were arrayed and expressed in HEK293 cells on new slides. This conformation study was performed both on fixed and live cells. The procedure and analyses were carried out as for described for the initial screen except that identical slides were treated, either after cell fixation or in the absence of cell fixation, with 5 μg/mL of each test antibody individually, 1 μg/mL Rituximab biosimilar (positive control), or no test molecule (AF647 anti-hIgG Fc secondary only) (n=2 slides per treatment for fixed cell screen and n=1 slide per treatment for live cell screen). Binding to target-expressing cells and untransfected cells was again assessed by fluorescence imaging.

In the confirmation screen, all 19 library hits (one of which is CD30L) and 2 control receptors (CD20 and EGFR) were over-expressed in HEK293 cells. As expected, the Rituximab biosimilar showed a med/strong intensity interaction with over-expressed CD20, validating the incubation conditions and detection system.

The binding of each of the tested anti-CD30L antibodies to CDH13 (hit #16 of the 19 hits) was very weak (very low confidence) in the library screen. Lack of reproducibility into the confirmation screen indicates that this was not a real interaction.

Twelve of the 19 library hits (#4 to #15 of the 19 hits, which are IGHG3, SLC22A23, CXCL12, IGF2, EDN2, IGF1, PAPPA, IGHG4, IGHG1, IGHG2, FCGR3A+FCER1G and FCGR3A+CD247, respectively) were bound by at least 1 of the control treatments (Rituximab biosimilar and/or no primary test molecule). These are therefore classed as non-specific, and included Fc gamma receptors (hit #s 14 and 15, FCGR3A+FCER1G and FCGR3A+CD247 respectively) which are presumably Fc-domain mediated and IGHG3 (hit #4) which is known to be bound directly by the secondary detection antibody. Hit #3 is TNFSF8 (CD30L), the primary target of the antibodies tested.

The signal intensities for the further 5 of the 19 library hits (hit #s 1, 2, 19, 20 and 21, which are FCGRIA, SLC38A4, GPR87, EPHA7, CACNA2D4) in the confirmation screen were classed as very weak (with the exception that clone 1 and clone 1 germlined binding to EPHA7 in live 293 cells was classified as weak) and, in 1 instance, only observed on 1 of the 2 replicate slides. For such hits, signals are so close to background levels that there is little to no confidence that they are real and/or specific. Thus no significance were attributed to such hits.

In contrast, the 4 representative anti-CD30L antibodies tested in this Example, including anti-CD30L antibody clone 1, clone 1 germlined, clone 2, and clone 2 germlined, all showed specific, strong intensity, interactions with TNFSF8 (CD30L), their primary target, on both fixed and live cell microarrays.

These data demonstrate high specificity of the 4 representative anti-CD30L antibodies tested in this Example, including anti-CD30L antibody clone 1, clone 1 germlined, clone 2, and clone 2 germlined for their primary target, TNFSF8 (CD30L).

### 5.9 Example 9: Additional Binding Studies and Comparison with Reference Anti-CD30L Antibodies

Representative anti-CD30L clone antibodies (clone 1, clone 1 germlined, clone 2, and clone 2 germlined) were also characterized for binding affinity to cynomolgus and human CD30L and for their ability to neutralize the interaction of CD30L to CD30. Binding of the representative anti-CD30L clones to recombinant cynomolgus (cyno) and human CD30L has been demonstrated by ELISA binding assays with purified human or cyno CD30L and by binding to CD30L expressing B16 or HEK293 cells in flow cytometry, with human or cyno CD30L expressed on HEK293 or B16 cells. In the B16 or HEK293 cell binding assays (cell binding assays as shown in FIG. 4A), the representative anti-CD30L clones were all shown to bind strongly to CD30L with nanomolar or sub-nanomolar affinity (Table 19). The representative binding and blocking curves for germlined clone 2 from such cell binding assays were shown in FIGS. 4B to 4E. In the ELISA binding assays with purified human or cyno CD30L (ELISA configuration shown in FIGS. 4F and 4G), the representative anti-CD30L clones were all shown to bind strongly to CD30L with picomolar affinity for recombinant human CD30L and nanomolar affinity for cyno CD30L (Table 20). The representative binding and blocking curves from the ELISA for germlined clone 2 were shown in FIGS. 4H and 41. When directly compared with the reference 1 (ref1) anti-CD30L (comprising HC of SEQ ID NO: 768 and LC of SEQ ID NO: 769), all 4 representative anti-CD30L clones had better IC₅₀, up to 2.74 fold better IC₅₀ (clone 2 germlined) against humanCD30L and up to 2.13 fold better IC₅₀ (clone 2 germlined) against cyno CD30L, at which the representative anti-CD30L clones blocked the binding of recombinant CD30-Fc to human or cyno CD30L expressed on HEK293 or B16 cells, (Table 19). In addition, these 4 representative anti-CD30L clones were tested in an IL-8 release dual cell assay, for cynoCD30L involving the K299 CD30+ cell line co-cultured with HEK293-cynoCD30L and for human CD30L involving K299 CD30+ cell line co-cultured with B16-humanCD30L cells. In such IL-8 release dual cell assay, K299 CD30+ release IL-8 upon ligation with and stimulation by the co-cultured CD30L expressing cells (FIG. 4J). Blocking of the CD30L-CD30 interaction can proportionally reduce the release of IL-8 and thus the amount of IL-8 released can be used as a readout for the effectiveness of antibody mediated blocking of CD30L-CD30 interactions between the co-cultured cells (FIG. 4J). All 4 representative anti-CD30L clones potently and effectively neutralized the cell-cell interaction mediated by CD30 and CD30L ligation and blocked IL-8 release, with IC₅₀ in the sub-nanomolar (clone 2 and clone 2 germlined) or nanomolar (clone 1 and clone 1 germlined) range (Table 21). The representative IL-8 release curves from the dual cell assay for germlined clone 2 were shown in FIGS. 4K and 4L. When directly compared with the reference 1 (ref1) anti-CD30L (comprising HC of SEQ ID NO: 768 and LC of SEQ ID NO: 769), all 4 representative anti-CD30L clones had better IC₅₀, up to 2.0 fold better IC₅₀ (clone 2) against humanCD30L and up to 2.6 fold better IC₅₀ (clone 2) against cyno CD30L, at which the representative anti-CD30L clones functionally blocked IL-8 release in the dual cell assay (Table 21).

TABLE 19
Summary of (1) the binding EC₅₀ of the representative anti-CD30L clones
to human or cyno CD30L expressed on HEK293 or B16 cells as indicated
and (2) the IC₅₀ at which the representative anti-CD30L clones blocked the
binding of recombinant CD30-Fc to human or cyno CD30L expressed on
HEK293 or B16 cells as indicated.

| | Human CD30L | | Cyno CD30L | |
|---|---|---|---|---|
| | | Ab-mediated | | Ab-mediated |
| | | blocking of | | blocking of |
| | | CD30-Fc | Binding | CD30-Fc to |
| | Binding | to B16- | to | HEK293- |
| | to B16- | huCD30L | HEK293- | cyCD30L |
| | rhCD30L | cells | cyCD30L | cells |
| Clones | EC₅₀ (nM) | IC₅₀ (nM) | EC₅₀ (nM) | IC₅₀ (nM) |
| Ref1 | 1.12 | 1.84 | 0.56 | 0.73 |
| CLONE_1 | 1.11 | 1.06 | 0.67 | 0.51 |
| CLONE_1_germ- | 1.12 | 1.05 | 0.58 | 0.54 |
| lined | | | | |
| CLONE_2 | 0.74 | 0.79 | 0.30 | 0.34 |
| CLONE_2_germ- | 0.64 | 0.67 | 0.33 | 0.34 |
| lined | | | | |

TABLE 20
Summary of (1) the binding EC₅₀ of the representative anti-
CD30L clones to purified human or cyno CD30L in an ELISA assay
and (2) the IC₅₀ at which the representative anti-CD30L
clones blocked the binding of recombinant CD30-Fc to purified
human or cyno CD30L in an ELISA assay.

| | Human CD30L | | Cyno CD30L | |
|---|---|---|---|---|
| | | Ab-mediated | | Ab-mediated |
| | Binding | blocking of | Binding | blocking of |
| | to | CD30-Fc to | to | CD30-Fc to |
| | rhCD30L | huCD30L | rcyCD30L | rcyCD30L |
| Clones | EC₅₀ (nM) | IC₅₀ (nM) | EC₅₀ (nM) | IC₅₀ (nM) |
| CLONE_1 | 0.05 | 2.14 | 0.07 | 1.58 |
| CLONE_1_germ- | 0.05 | 1.85 | 0.07 | 1.71 |
| lined | | | | |
| CLONE_2 | 0.04 | 1.35 | 0.05 | 1.28 |
| CLONE_2_germ- | 0.03 | 1.92 | 0.05 | 1.53 |
| lined | | | | |

TABLE 21
Summary of the IC₅₀ at which the representative anti-CD30L
clones blocked the IL-8 release in a dual cell assay.

| | Inhibition of | Inhibition of |
|---|---|---|
| | IL-8 release in a | IL-8 release in a |
| | dual cell assay (B16- | dual cell assay (HEK293- |
| | huCD30L cells + | cyCD30L cells + |
| | K299 cells) | K299 cells) |
| Clones | IC₅₀ (nM) | IC₅₀ (nM) |
| Ref1 | 1.61 | 0.79 |
| CLONE_1 | 1.43 | 0.39 |
| CLONE_1_germ- | 1.29 | 0.43 |
| lined | | |
| CLONE_2 | 0.81 | 0.31 |
| CLONE_2_germ- | 0.89 | 0.41 |
| lined | | |

To more precisely define and compare the binding affinities (dissociation equilibrium constant, K_{D}), the association rate constant (on rate, kₒₙ), and the dissociation rate constant (off rate, k_{off}), the kinetic exclusion assay (KinExA) was performed. KinExA measures the equilibrium binding affinity and kinetics between unmodified binding molecules in solution. For affinity analysis, the equilibrium dissociation constant, K_{D}, is experimentally determined and reflects the strength of the binding interaction. The rate of association, kₒₙ, is also experimentally determined, while the rate of dissociation, k_{off}, is calculated based on the equation: k_{off}=K_{d}×kₒₙ.

A K_{D} analysis requires immobilization of one interaction partner to a solid phase which is then used as a probe to capture the other interaction partner, the constant binding partner (Fab). For each experiment, CD30L antigen is titrated in a background of the Fab and allowed to reach equilibrium. The solutions are then briefly exposed to the solid phase and a portion of free Fab is captured. The captured Fab is then labeled with a fluorescent secondary molecule. The short contact time with the solid phase is less than the time needed for dissociation of the pre-formed complex in solution, thus competition between the solution and the solid phase titrated binding partner is kinetically excluded.

The signals generated from the captured Fab, which are directly proportional to the concentration of free Fab in the equilibrated samples, are used to determine the K_{D} value. The KinExA Pro software performs a least squares analysis on the measured data to fit optimal solution for the K_{D} and the activity of the Fab to a curve representative of a 1:1 reversible bi-molecular interaction.

Using a KinExA 3200 instrument, the affinity and kinetics were measured for the Fab of a representative anti-CD30L, clone 2 germlined, to human CD30L. The K_{D}, kₒₙ, and k_{off} were shown in Table 22.

TABLE 22
Binding of Fab of clone 2 germlined to human CD30L.

| | | |
|---|---|---|
| K_{D} | 90.5 | pM |
| 95% Confidence Interval | 65.4 to 125 | pM |

| | |
|---|---|
| On Rate (kₒₙ) (M⁻¹s⁻¹) | 1.55 × 10⁶ |
| 95% Confidence Interval (M⁻¹s⁻¹) | 1.50 × 10⁶ to 1.61 × 10⁶ |
| Off Rate (k_{off}) (s⁻¹) | 1.41 × 10⁻⁴ |

Using a KinExA 3200 instrument, the affinity and kinetics were measured for the Fab of a representative anti-CD30L, clone 2 germlined, to cyno CD30L. The K_{D}, kₒₙ, and k_{off} were shown in Table 23.

TABLE 23
Binding of the Fab of clone 2 germlined to cyno CD30L.

| | | |
|---|---|---|
| K_{D} | 122 | pM |
| 95% Confidence Interval | 51.3 to 244 | pM |

| | |
|---|---|
| On Rate (kₒₙ) (M⁻¹s⁻¹) | 3.1 × 10⁶ |
| 95% Confidence Interval (M⁻¹s⁻¹) | 2.45 × 10⁶ to 3.89 × 10⁶ |
| Off Rate (k_{off}) (s⁻¹) | 3.78 × 10⁻⁴ |

As a direct comparison, using a KinExA 3200 instrument, the affinity and kinetics were measured for the Fab of the reference 1 (ref1) anti-CD30L (comprising VH of SEQ ID NO:766 and VL of SEQ ID NO: 767), to human CD30L. The K_{D}, kₒₙ, and k_{off} for this reference 1 anti-CD30L were shown in Table 24.

TABLE 24
Binding of Fab of the reference 1 anti-CD30L to human CD30L.

| | | |
|---|---|---|
| K_{D} | 377 | pM |
| 95% Confidence Interval | 289 to 489 | pM |

| | |
|---|---|
| On Rate (kₒₙ) (M⁻¹s⁻¹) | 9.37 × 105 |
| 95% Confidence Interval (M⁻¹s⁻¹) | 7.56 × 10⁵ to 1.09 × 10⁶ |
| Off Rate (k_{off}) (s⁻¹) | 3.53 × 10⁻⁴ |

Therefore, the representative anti-CD30L provided herein, clone 2 germlined, has picomolar affinity for both human and cyno CD30L and has 4.2 fold higher K_{D} than the reference 1 anti-CD30L in a head-to-head comparison with the reference 1 anti-CD30L.

### 5.10 Example 10. Formulation Studies of a Representative Anti-CD30L Clone (Germlined Clone 2)

This disclosure provides formulation studies on a representative anti-CD30L clone (germlined clone 2). Two manufactured lots of the full-length anti-CD30L germlined clone 2 were used in the formulation studies. One lot was used for solubility study and pH/buffer study and the other lot for the excipient and surfactant study and oxidation study.

### 5.10.1 Analytical Methods Used in the Formulation Studies

Appearance: The appearance of all samples, including clarity, color and visible particles, was examined against black and white background using YB-2 Clarity Detector.

### Protein Conc. (A280):

Protein conc. was determined by a Thermo Ultraviolet (UV) spectrophotometer throughout the study except for the pH/buffer study. A280 value for each sample was determined and the extinction coefficient used in this evaluation was 1.49 (mg/mL)⁻¹·cm⁻¹.

Protein concentration in the pH/buffer study was measured using a Big Lunatic high throughput UV spectrophotometer. The extinction coefficient used in all evaluation studies was 1.49 AU*mL*mg⁻¹*cm⁻¹.

pH:

Sample pH was measured using a pH meter with a glass electrode. All measurements were repeated twice, and an average result was described.

### Osmolality:

Osmolality was measured using an Osmometer. Before and after tests, accuracy of the osmometer was confirmed with a Clinitrol 290 mOsm/kg reference solution.

### SE-UPLC:

SE-UPLC is a purity analysis method that separates proteins based on their size. The procedure of SE-UPLC analysis was as follows: sample was diluted to 10 mg/mL with mobile phase before SE-UPLC analysis if the sample concentration was above 10 mg/mL, then 10 μg sample was injected into SE-UPLC. The equipment used was the Agilent UPLC system with a SEC column (4.6×150 mm, 1.7 μm) and a UV detector (detection wavelength: 280 nm). The sampler temperature was set to 5±3° C. and the column oven temperature was set as 25±3° C. Mobile phase was 50 mM phosphate buffer, 300 mM Sodium Chloride, pH 6.8±0.1. An isocratic gradient was applied for 20 min at a flow rate of 0.4 mL/min with next injection delay of 8 min. All the raw data was processed with Empower 3.

### HIAC:

A Hach Particulate Analyzer was utilized to measure the size and counts of sub-visible particles in a bio-safety hood. To avoid introducing air bubbles and interference during examination, all samples were held in the bio-safety hood for at least 0.5 hour before testing. Each sample was tested for four consecutive runs, 0.45 mL each. The results were presented as average number of particles of ≥2 μm, ≥10 μm and ≥25 μm per mL (method conforms to USP <788> Particulate matter in injections).

iCIEF:

iCIEF is a purity analysis method used to monitor the percentage of charge variant species. The isoelectric point (pI) is an intrinsic property of a specific protein molecule and is the pH at which the protein molecule does not carry net electrical charge. Under an external electric field, the charge variants move along a continuous pH gradient formed by ampholytes and stop at where the pH equals its pI. For the anti-CD30L antibody (germlined clone 2), 20 μg sample was mixed with 80 μL master mix, which was composed of 0.5 μL of pI 7.05 marker, 0.5 μL of pI 9.91 marker, 1 μL of Pharmalyte 3-10, 3 μL of Pharmalyte 8-10.5, 35 μL of 1% methyl cellulose, 37.5 μL of 8 M urea solution and 2.5 μL of ultrapure water. The mixture was then analyzed with the iCE3 Capillary Isoelectric Focusing Analyzer equipped with a fluorocarbon (FC)-coated iCIEF cartridge. The sample was focused 1 minute at 1500 V and 8 minutes at 3000 V, respectively. Detection wavelength was set at 280 nm to evaluate the charge variants distribution in different pI range.

### Caliper-SDS (Non-reduced and Reduced):

Caliper-SDS was performed on a PerkinElmer Caliper automated electrophoresis. For non-reduced (NR), the sample denaturing solution was prepared by mixing sample buffer with 10% sodium dodecyl sulfate (SDS) and 100 mM N-Ethylmaleimide (NEM) at the volume ratio of 25:2.5:1. While for reduced (R), the sample denaturing solution was prepared by mixing sample buffer with 10% SDS and 1 M dithiothreitol at the volume ratio of 25:2.5:1.

Before running, 2 μL of diluted samples (diluted to 1 mg/mL) or water (for blank control) was mixed with 7 μL of the sample denaturing solution. For standard control, 8 μL of diluted reference standard (diluted to 1 mg/mL) was mixed with 28 μL of sample denaturing solution. In addition, 12 μL of protein ladder (supplied with the Caliper Reagent Kit) was prepared as well. Subsequently, these prepared vials were incubated at 70° C. for 10 minutes and then mixed well. After that, 35 μL of water was added to each sample and blank control vial, 140 μL of water to the standard vial and 120 μL of water to the ladder vial. Then all the vials were vortexed and centrifuged for a few seconds. 42 μL of the prepared samples and control solutions was transferred individually to a 96-well plate and centrifuged at 4000 rpm for 20 minutes. Then, the sample plate was placed onto a Labchip GXII's plate holder. Thereafter, prepared samples and control solutions were loaded, stained, separated and detected in the HT Protein Express LabChip filled with destain-gel, gel-dye and marker. At last, the raw data was analyzed with LabChip GX Reviewer software.

Differential Scanning calorimetry (DSC):

DSC is a thermos-analytical technique used to characterize the thermal stability of protein samples and assess conformational differences between them. Measurements were performed on MicroCal PEAQ DSC (Malvern) for thermal transition midpoint (Tm) and onset of unfolding (TOnset) testing. Samples were diluted to 1 mg/mL with the reference buffer. 400 μL of respective reference buffers was added into the odd-numbered wells of a 96-well plate and 400 μL of samples was added into the even-numbered wells of the same plate. Experimental parameters were set such that the scan temperature ramped from 10 to 95° C. at a scan rate of 200° C./h. Data analysis was performed in MicroCal PEAQ-Capillary DSC automated data analysis software.

### Viscosity:

The viscosity of protein sample was measured by DHR-2 Hybrid Rheometer at 25° C. The sample volume for testing was 0.6 mL and only one test was performed. The scan shear rate ramped from 10-1500 l/s with data read per 10 decades. At least 5 continuous data points within a linear range were selected to calculate viscosity for Newtonian fluid.

### PEG Precipitation Test:

PEG precipitation test is a high-throughput method for determining protein solubility in different formulation buffers. Sample was diluted to the concentration of 4 mg/mL. 1 part of protein sample, 2 parts of 5%, 10%, 15%, 20%, 25%, 30%, 35%, 40% PEG solution (w/v, dissolved in water) and 1 part of 60 mM corresponding buffer were mixed. The mixture solution was centrifuged with the speed at 4000 rpm for 30 minutes and then tested for protein conc. in supernatant using Big Lunatic.

### Post-translational Modification (PTM):

For reduced peptide mapping experiment, the protein was carried denatured with guanidine hydrochloride (Gdn-HCl) in Tris-HCl buffer, reduced by dithiothreitol (DTT), and followed by cysteine-alkylation with iodoacetamide (IAM). After sample clean-up, the protein was digested with Lys-C/trypsin sequential digestion with Lys-C/trypsin treatment to obtain peptides suitable for subsequent analysis. The online LC-MS analysis was performed on an Agilent/UHPLC 1290 system coupled to an Orbitrap mass spectrometer. The proteolytic peptides were separated by reversed phase liquid chromatography with an Agilent/Poroshell SB-C18 column, and the UV chromatograms were acquired with the detector set at wavelength of 214 nm. The peptides were further detected by the mass spectrometer operating with full MS scan followed by Tandem Mass Spectrometry (MS/MS) scans.

The characteristics of the peaks were confirmed by comparing the chromatographic profiles, considering the number of detected peaks, retention time, and peak intensity. The mass raw data including high resolution MS spectra and MS/MS spectra were analyzed using Protein Metrics Inc. (PMI)/Byonic search engine against the theoretical sequences. The measured masses of the peptides were compared to the theoretical masses to verify the correct identification of each peptide. Furthermore, potential mutation or post-translational modification (PTM) sites can be determined and manually confirmed. The PTM ratio was semi-quantitated by Extracted Ion Chromatograms (EICs) using the following equation:

$$EIC ratio = \frac{EIC {area}_{modified peptides}}{\sum EIC {area}_{modified peptides} + EIC {area}_{unmodified peptides}} \times 100 \%$$

The modified peptides with PTM ratio greater than 1.0% were identified.

### 5.10.2 Brief Overview of the Formulation Studies

In these formulation studies, protein solubility was evaluated, the impact of pH/buffer systems, excipients and surfactant strength on the anti-CD30L antibody stability were evaluated, and the effect of three oxidants on protein quality was also evaluated.

Briefly, for solubility study, three protein conc. from 100 to 200 mg/mL under stress conditions (40° C. for up to 2 weeks), one conc. (250 mg/mL) under 2-8° C. were investigated in the solubility study for their viscosity and stability performance. Appearance, pH, protein conc., viscosity, SE-UPLC, iCIEF and caliper-SDS (NR & R) were also tested. In the pH/buffer studies, twelve pH/buffer systems under stress conditions (40° C. for up to 2 weeks) were investigated, and the appearance, pH, protein conc., SE-UPLC, iCIEF, DSC, PEG precipitation test and caliper-SDS (NR & R) were tested. For excipients/surfactant strength studies, different types of excipients (sucrose, L-proline, ArgHCl, etc.), different types and concentrations of surfactants (0.02%-0.04% PS80, 0.02% PS20 and 0.05% poloxamer188 (P188)) were evaluated under stress conditions (40° C. for up to 4 weeks, agitation at 300 rpm for 3 days, and freeze/thaw for up to 5 cycles). Appearance, pH, protein conc., osmolality, viscosity, SE-UPLC, caliper-SDS (NR & R), iCIEF, HIAC and DSC were also evaluated in the excipients/surfactant strength study. For oxidation study, the stability of two formulations with or without L-Methionine under oxidation stress of three oxidants (0.1% H2O2, 0.2% AAPH and 0.5% tBHP) was investigated, and appearance, pH, protein conc., SE-UPLC, iCIEF, caliper-SDS (NR & R) and PTM were tested.

### 5.10.3 Solubility Study

The representative full-length anti-CD30L antibody (germlined clone 2) was first concentrated to over 250 mg/mL. Stock solutions of 40% (w/w) sucrose and 5% (w/w) polysorbate 80 (PS80) were used to prepare the formulations listed in Table 28. The amounts of anti-CD30L, excipient stock solutions and surfactant stock solution were calculated, measured and well-mixed based on the formulation recipes. Each of the prepared samples was filtered with a 0.22 μm polyvinylidene fluoride (PVDF) filter and filled into 2 ml glass vials (1 mL per vial), then stoppered, capped and labeled immediately. All procedures were carried out in a bio-safety hood. The various protein concentrations and the corresponding formulations tested in this study are listed in Table 28

TABLE 28
Candidates list of solubility study

| | | Protein Conc. |
|---|---|---|
| No. | Formulation | (mg/mL) |
| Fs01 | 20 mM Histidine, 6% (w/v) sucrose, 0.04% | 100 |
| Fs02 | PS80, pH 6.0 | 150 |
| Fs03 | | 200 |
| Fs04 | 20 mM Histidine, pH 6.0 | 250 |

Table 29 shows the sampling and testing for the solubility study. Fs01-Fs03 samples were stored at 40° C. for up to 2 weeks and Fs04 sample was stored at 2-8° C. for 24 hours. Samples were taken at each time point and kept at −70° C. before analysis. Testing items including appearance, pH, protein conc., SE-UPLC, iCIEF, Caliper-SDS (NR & R), and viscosity were performed in this study.

TABLE 29
Sampling and testing plan of solubility study

40° C.

| No. | T0 | 1 W | 2 W |
|---|---|---|---|
| Fs01-Fs03 | X, Y, Z | X, Y | X, Y |

| | | |
|---|---|---|
| Fs04 | X, Z | 2-8° C., 24 H |
| | | X |

X = Appearance, pH, protein conc.
Y = SE-UPLC, iCIEF, Caliper-SDS (NR & R)
Z = Viscosity

### Appearance and Viscosity:

Table 30 provides the data from the appearance and viscosity testing for the solubility study. For Fs01 (100 mg/mL), Fs02 (150 mg/mL) and Fs03 (200 mg/mL), all samples remained slightly yellow, slightly opalescent and free of visible particles after stored at 40° C. for up to 2 weeks. For Fs04 (250 mg/mL) without addition of sucrose and PS80, samples were slightly yellow, slightly opalescent and not essentially free of visible particles (more than 5 particles were found) at T0 and after stored at 2-8° C. for 24 hours. As protein conc. increased from 100 mg/mL to 250 mg/mL, viscosity increased significantly from 3.34 cp to 92.35 cp. The viscosity reached over 20 cp for the 200 mg/mL samples.

TABLE 30
Solubility study: appearance and viscosity

| | Appearance | | | Viscosity |
|---|---|---|---|---|
| Formulation | | 40° C. | | (cp) |
| No. | T0 | 1 W | 2 W | (25° C.) |
| Fs01 | SY, SO, FP | SY, SO, FP | SY, SO, FP | 3.34 |
| Fs02 | SY, SO, FP | SY, SO, FP | SY, SO, FP | 7.38 |
| Fs03 | SY, SO, FP | SY, SO, FP | SY, SO, FP | 22.45 |

| | | | |
|---|---|---|---|
| Fs04 | T0 | 2-8° C.-24 h | 92.35 |
| | SY, SO, NEFP | SY, SO, NEFP | |

Abbreviation: SY = slightly yellow, SO = slightly opalescent, FP = free of visible particles, NEFP (not essentially free of visible particles, >5).

### pH and Protein Concentration:

Table 31 provides the results of the pH and protein conc. testing for the solubility study. For Fs01 (100 mg/mL), Fs02 (150 mg/mL) and Fs03 (200 mg/mL), no substantial change in pH and protein conc. was observed after incubation at 40° C. for up to 2 weeks. For Fs04 (250 mg/mL), no substantial change in pH and protein conc. was observed after incubation at 2-8° C. for 24 hours. During the sample preparation, pH shift was observed after concentration by centrifugation due to donnan effect, and further shifted under higher protein concentration. The pH of the anti-CD30L antibody used for solubility study is pH6.0 and it was increased to 6.2 for Fs01 (100 mg/mL) and 6.4 for Fs04 (250 mg/mL).

TABLE 31
Solubility study: pH and protein conc.

| | pH | | | Protein Conc. * (mg/mL) | | |
|---|---|---|---|---|---|---|
| | | 40° C. | | | 40° C. | |
| Formulation No. | T0 | 1 W | 2 W | T0 | 1 W | 2 W |
| Fs01 | 6.2 | 6.1 | 6.1 | 102.4 | 102.3 | 102.1 |
| Fs02 | 6.3 | 6.2 | 6.2 | 151.6 | 150.9 | 155.7 |
| Fs03 | 6.3 | 6.3 | 6.3 | 209.8 | 204.2 | 205.0 |

| | | | | |
|---|---|---|---|---|
| Fs04 | T0 | 5° C.-24 h | T0 | 5° C.-24 h |
| | 6.4 | 6.4 | 255.1 | 250.8 |

* samples were 10-fold diluted before protein conc. test

### SE-UPLC:

Table 32 shows the data summary of SE-UPLC testing results for the solubility study. decrease in % main peak (% MP)(about 1.0-2.0%) was observed after incubation at 40° C. for 2 weeks, which mainly came from increases in % HMW. Higher percentage of aggregation was observed in high-conc. samples and longer thermal stress incubation.

TABLE 32
Solubility study: SE-UPLC

| | SE-UPLC | | | | | | | | |
|---|---|---|---|---|---|---|---|---|---|
| | Main peak (%) | | | HMW (%) | | | LMW (%) | | |
| | | 40° C. | | | 40° C. | | | 40° C. | |
| | | 1 | 2 | | 1 | 2 | | 1 | 2 |
| Formulation No. | T0 | W | W | T0 | W | W | T0 | W | W |
| Fs01 | 99.4 | 98.9 | 98.4 | 0.6 | 0.9 | 1.4 | 0.0 | 0.2 | 0.3 |
| Fs02 | 99.4 | 98.4 | 98.0 | 0.6 | 1.4 | 1.7 | 0.0 | 0.2 | 0.3 |
| Fs03 | 99.4 | 97.9 | 97.4 | 0.6 | 1.8 | 2.2 | 0.0 | 0.2 | 0.3 |

Abbreviation: HMW (high molecular weight); LMW (low molecular weight)

iCIEF:

Table 33 shows the data summary of iCIEF testing results for the solubility study. Decrease (12.5˜15.0%) in % MP was observed after incubation at 40° C. for 2 weeks, which mainly came from increases in % Acidic peaks (% AP). More decrease of % MP was found with thermal incubation time. No clear correlation between iCIEF change and protein conc. was observed.

TABLE 33
Solubility study: iCIEF

| | iCIEF | | | | | | | | |
|---|---|---|---|---|---|---|---|---|---|
| | Main peak (%) | | | Acidic peaks (%) | | | Basic peaks (%) | | |
| | | 40° C. | | | 40° C. | | | 40° C. | |
| | | 1 | 2 | | 1 | 2 | | 1 | 2 |
| Formulation No. | T0 | W | W | T0 | W | W | T0 | W | W |
| Fs01 | 72.9 | 65.3 | 57.9 | 14.4 | 23.2 | 31.0 | 12.7 | 11.5 | 11.0 |
| Fs02 | 72.1 | 64.1 | 58.2 | 15.0 | 24.3 | 30.1 | 13.0 | 11.6 | 11.7 |
| Fs03 | 71.8 | 65.4 | 59.3 | 15.3 | 23.3 | 28.6 | 12.9 | 11.2 | 12.1 |

### Caliper-SDS (NR & R):

Caliper-SDS (NR & R) testing results for the solubility study are shown in Table 34. For Caliper-SDS-NR, decrease (1.9˜2.0%) in purity was observed after incubation at 40° C. for 2 weeks. No clear correlation between Caliper-SDS-NR change and protein conc. was observed. For Caliper-SDS-R, no substantial change was observed after incubation at 40° C. for 2 weeks.

TABLE 34
Solubility study: Caliper-SDS (NR & R)

| | Caliper-SDS-NR (Purity %) | | | Caliper-SDS-R (LC + HC) % | | |
|---|---|---|---|---|---|---|
| | | 40° C. | | | 40° C. | |
| Formulation No. | T0 | 1 W | 2 W | T0 | 1 W | 2 W |
| Fs01 | 98.1 | 97.0 | 96.2 | 99.6 | 99.5 | 99.4 |
| Fs02 | 98.1 | 97.1 | 96.1 | 99.5 | 99.4 | 99.4 |
| Fs03 | 98.1 | 97.0 | 96.2 | 99.6 | 99.5 | 99.2 |

Abbreviation: LC (light chain); HC (heavy chain)

In summary, the solubility study investigated formulation stability and viscosity with three different protein conc. (100 mg/mL, 150 mg/mL and 200 mg/mL) under stress conditions (40° C. for up to 2 weeks) and one higher protein conc. (250 mg/mL) under 2-8° C. for 24 hours. No substantial change was observed in appearance, pH and protein conc. throughout the study. pH shift was observed after concentration by centrifugation during sample preparation. As protein conc. increased from 100 mg/mL to 250 mg/mL, viscosity increased from 3.34 cp to 92.35 cp. The viscosity reached to over 20 cp for 200 mg/mL samples. More aggregation was observed at higher protein conc. from SE-UPLC results. No substantial change was observed in iCIEF and Caliper-SDS (R & NR) between different protein conc. The result suggests that the anti-CD30L antibody provided here in can achieve high concentration (e.g. ≥150 mg/ml or ≥200 mg/ml) in the formulations.

5.10.4 pH/Buffer Study

The pH/buffer study aimed at selecting the optimal pH/buffer system that can stabilize the protein. The pH/buffer study was conducted under stress condition (40° C. incubation).

The representative anti-CD30L antibody (germlined clone 2) was first buffer-exchanged into 12 buffers including 20 mM acetate buffer (Ace, pH 4.5, pH 5.0, pH 5.5), 20 mM succinate buffer (Suc, pH 5.0, pH 5.5, pH 6.0), 20 mM histidine buffer (His, pH 5.5, pH 6.0, pH 6.5), and 20 mM phosphate buffer (PB, pH 6.5, pH 7.0, pH 7.5), and then concentrated to 20 mg/mL protein concentration, respectively (Table 35). Each of the prepared samples was filtered with a 0.22 PVDF filter and filled into 2 ml glass vials (1 mL per vial), then stoppered, capped and labeled immediately. All procedures were carried out in a bio-safety hood. The buffers and corresponding pH are shown in Table 35.

TABLE 35
Formulation candidates list of pH/buffer study

| Formulation No. | Buffer System | Target pH |
|---|---|---|
| F1 | 20 mM Acetate | 4.5 |
| F2 | | 5.0 |
| F3 | | 5.5 |
| F4 | 20 mM Succinate | 5.0 |
| F5 | | 5.5 |
| F6 | | 6.0 |
| F7 | 20 mM Histidine | 5.5 |
| F8 | | 6.0 |
| F9 | | 6.5 |
| F10 | 20 mM PB | 6.5 |
| F11 | | 7.0 |
| F12 | | 7.5 |

Table 36 shows the sampling and testing for the pH/buffer study. Samples were stored at 40° C. for up to 2 weeks, and then taken at each time point and kept at −70° C. before analysis. Testing items including appearance, pH, protein conc., SE-UPLC, iCIEF, Caliper-SDS (NR & R), DSC and PEG precipitation test were performed in this study.

TABLE 36
Sampling and testing plan of pH/buffer study

40° C.

| Formulation No. | T0 | 1 W | 2 W |
|---|---|---|---|
| F1-F12 | X, Y | X | X |

X = Appearance, pH, protein conc., SE-UPLC, iCIEF, Caliper-SDS (NR & R),
Y = DSC, PEG precipitation test

### Appearance:

Table 37 provides the results from the appearance testing for the pH/buffer study. All samples remained colorless, slightly opalescent and essentially free of visible particles when stored at 40° C. for 2 weeks, except for F09-F12. For F09 (His 6.5), F10 (PB 6.5) and F11 (PB 7.0), a few particles were observed, for F12 (PB 7.5), many particles were observed.

TABLE 37A
pH/buffer study: appearance

| | Appearance | | |
|---|---|---|---|
| Formulation | | 40° C. | |
| No. | T0 | 1 W | 2 W |
| F1 (Ac 4.5) | C, SO, FP | C, SO, FP | C, SO, FP |
| F2 (Ac 5.0) | C, SO, FP | C, SO, EFP (3) | C, SO, FP |
| F3 (Ac 5.5) | C, SO, FP | C, SO, FP | C, SO, EFP (2) |
| F4 (Suc 5.0) | C, SO, FP | C, SO, FP | C, SO, FP |
| F5 (Suc 5.5) | C, SO, FP | C, SO, FP | C, SO, FP |
| F6 (Suc 6.0) | C, SO, FP | C, SO, FP | C, SO, FP |
| F7 (His 5.5) | C, SO, FP | C, SO, FP | C, SO, FP |
| F8 (His 6.0) | C, SO, FP | C, SO, FP | C, SO, FP |
| F9 (His 6.5) | C, SO, FP | C, SO, FP | C, SO, NEFP |
| | | | (a few) |
| F10 (PB 6.5) | C, SO, FP | C, SO, NEFP | C, SO, NEFP |
| | | (a few) | (a few) |
| F11 (PB 7.0) | C, SO, NEFP | C, SO, NEFP | C, SO, NEFP |
| | (a few) | (a few) | (a few) |
| F12 (PB 7.5) | C, SO, NEFP | C, SO, NEFP | C, SO, NEFP |
| | (many) | (many) | (many) |

Abbreviation: C = colourless, SO = slightly opalescent, FP = free of visible particles, EFP (essentially free of visible particles, ≤5), NEFP (not essentially free of visible particles, >5).

### pH and Protein Concentration:

Table 37 shows the results of pH and protein conc. testing for the pH/buffer study. No substantial change was observed in pH and protein conc. after incubation at 40° C. for 2 weeks.

TABLE 37B
pH/buffer study: pH and protein conc.

| | pH | | | Protein Conc. (mg/mL) | | |
|---|---|---|---|---|---|---|
| | | 40° C. | | | 40° C. | |
| Formulation No. | T0 | 1 W | 2 W | T0 | 1 W | 2 W |
| F1 (Ac 4.5) | 4.5 | 4.5 | 4.6 | 19.7 | 19.7 | 19.7 |
| F2 (Ac 5.0) | 5.0 | 5.0 | 5.0 | 19.7 | 19.6 | 19.6 |
| F3 (Ac 5.5) | 5.6 | 5.6 | 5.6 | 19.7 | 19.7 | 19.7 |
| F4 (Suc 5.0) | 5.0 | 5.0 | 5.1 | 19.7 | 19.7 | 19.6 |
| F5 (Suc 5.5) | 5.5 | 5.5 | 5.5 | 19.6 | 19.7 | 19.6 |
| F6 (Suc 6.0) | 6.0 | 6.0 | 6.1 | 19.9 | 19.9 | 19.9 |
| F7 (His 5.5) | 5.5 | 5.5 | 5.5 | 19.7 | 19.8 | 19.8 |
| F8 (His 6.0) | 6.1 | 6.1 | 6.1 | 19.8 | 19.9 | 19.8 |
| F9 (His 6.5) | 6.6 | 6.5 | 6.6 | 19.7 | 19.7 | 19.7 |
| F10 (PB 6.5) | 6.5 | 6.4 | 6.5 | 19.4 | 19.6 | 19.5 |
| F11 (PB 7.0) | 6.9 | 6.9 | 6.9 | 19.7 | 19.7 | 19.5 |
| F12 (PB 7.5) | 7.3 | 7.3 | 7.3 | 19.8 | 19.7 | 19.7 |

### SE-UPLC:

Table 38 shows the data of SE-UPLC testing results for the pH/buffer study. Decrease in % MP (about 0.3˜1.5%) was observed after incubation at 40° C. for 2 weeks. From the SE-UPLC results, the histidine buffer system performed better than the other three buffer system. The performance of the acetate buffer system ranked the second, followed by succinate buffer system and PB system.

TABLE 38
pH/buffer study: SE-UPLC

| | SE-UPLC | | | | | | | | |
|---|---|---|---|---|---|---|---|---|---|
| | Main peak (%) | | | HMW (%) | | | LMW (%) | | |
| Formulation | | 40° C. | | | 40° C. | | | 40° C. | |
| No. | T0 | 1 W | 2 W | T0 | 1 W | 2 W | T0 | 1 W | 2 W |
| F1 (Ac 4.5) | 99.6 | 99.3 | 99.2 | 0.4 | 0.3 | 0.2 | 0.0 | 0.4 | 0.6 |
| F2 (Ac 5.0) | 99.6 | 99.3 | 99.2 | 0.4 | 0.4 | 0.3 | 0.0 | 0.3 | 0.5 |
| F3 (Ac 5.5) | 99.6 | 99.2 | 98.9 | 0.4 | 0.5 | 0.5 | 0.0 | 0.3 | 0.6 |
| F4 (Suc 5.0) | 99.7 | 99.3 | 99.0 | 0.3 | 0.4 | 0.4 | 0.0 | 0.3 | 0.5 |
| F5 (Suc 5.5) | 99.6 | 99.2 | 98.9 | 0.4 | 0.5 | 0.6 | 0.0 | 0.3 | 0.5 |
| F6 (Suc 6.0) | 99.6 | 98.8 | 98.5 | 0.4 | 0.8 | 0.8 | 0.0 | 0.4 | 0.7 |
| F7 (His 5.5) | 99.7 | 99.5 | 99.4 | 0.3 | 0.3 | 0.3 | 0.0 | 0.2 | 0.3 |
| F8 (His 6.0) | 99.7 | 99.5 | 99.4 | 0.3 | 0.3 | 0.3 | 0.0 | 0.2 | 0.3 |
| F9 (His 6.5) | 99.7 | 99.5 | 99.3 | 0.3 | 0.3 | 0.4 | 0.0 | 0.2 | 0.3 |
| F10 (PB 6.5) | 99.6 | 98.7 | 98.2 | 0.4 | 0.8 | 1.0 | 0.0 | 0.5 | 0.8 |
| F11 (PB 7.0) | 99.6 | 98.5 | 98.0 | 0.4 | 1.1 | 1.3 | 0.0 | 0.5 | 0.8 |
| F12 (PB 7.5) | 99.5 | 98.3 | 97.6 | 0.5 | 1.2 | 1.5 | 0.0 | 0.5 | 0.8 |

iCIEF:

Table 39 shows the data summary of iCIEF testing results for the pH/buffer study. A decline in the % MP of up to 28.7% for PB buffer system and 9.7% for others was seen in all buffer systems after storage at 40° C. for 2 weeks. The histidine buffer system performed the best, followed by the acetate buffer system and the succinate buffer system. The PB system showed the worst performance especially under higher pH.

TABLE 39
pH/buffer study: iCIEF

| | iCIEF | | | | | | | | |
|---|---|---|---|---|---|---|---|---|---|
| | Main peak (%) | | | Acidic peaks (%) | | | Basic peaks (%) | | |
| Formulation | | 40° C. | | | 40° C. | | | 40° C. | |
| No. | T0 | 1 W | 2 W | T0 | 1 W | 2 W | T0 | 1 W | 2 W |
| F1 (Ac 4.5) | 71.6 | 65.3 | 58.1 | 15.1 | 21.3 | 28.1 | 13.4 | 13.5 | 13.9 |
| F2 (Ac 5.0) | 72.6 | 65.0 | 58.6 | 14.8 | 21.9 | 28.4 | 12.6 | 13.1 | 13.0 |
| F3 (Ac 5.5) | 71.6 | 66.8 | 60.7 | 15.7 | 21.1 | 28.1 | 12.7 | 12.0 | 11.2 |
| F4 (Suc 5.0) | 70.7 | 62.1 | 55.5 | 16.0 | 24.4 | 32.4 | 13.4 | 13.5 | 12.1 |
| F5 (Suc 5.5) | 71.4 | 63.9 | 58.3 | 15.7 | 23.5 | 29.0 | 12.9 | 12.6 | 12.7 |
| F6 (Suc 6.0) | 71.8 | 65.9 | 60.9 | 15.3 | 21.4 | 27.2 | 12.9 | 12.7 | 11.9 |
| F7 (His 5.5) | 71.1 | 66.3 | 60.4 | 16.1 | 21.1 | 26.9 | 12.8 | 12.7 | 12.7 |
| F8 (His 6.0) | 71.2 | 67.5 | 61.5 | 16.0 | 19.9 | 26.2 | 12.7 | 12.6 | 12.3 |
| F9 (His 6.5) | 72.0 | 65.6 | 60.8 | 15.5 | 21.4 | 26.0 | 12.5 | 13.0 | 13.2 |
| F10 (PB 6.5) | 72.3 | 62.7 | 56.4 | 14.9 | 24.0 | 30.4 | 12.8 | 13.3 | 13.2 |
| F11 (PB 7.0) | 71.6 | 61.1 | 51.0 | 15.6 | 25.1 | 35.2 | 12.8 | 13.9 | 13.8 |
| F12 (PB 7.5) | 70.7 | 53.0 | 42.0 | 15.9 | 33.1 | 44.4 | 13.4 | 13.9 | 13.7 |

### Caliper-SDS (NR & R):

Caliper-SDS (NR & R) testing results for the pH/buffer study is shown in Table 40. For Caliper-SDS-NR, the purity of all samples decreased with the increase of incubation time at 40° C. for up to 2 weeks. F10-F12 (PB buffer system) showed a faster and greater extent of decline (>4.5% by 2 weeks) than the samples in other three buffer systems. Histidine buffer system showed the best performance. For Caliper-SDS-R, minimal changes were seen in all samples. Decrease of purity (LC+HC, %) for no more than 1.1% was observed for all samples. Higher extent of decrease was observed in Caliper-SDS-NR than those in Caliper-SDS-R, which indicated certain degree of breakage on disulfide bonds.

TABLE 40
pH/buffer study: Caliper-SDS (NR & R)

| | Caliper-SDS-NR (Purity %) | | | Caliper-SDS-R (LC + HC) % | | |
|---|---|---|---|---|---|---|
| | | 40° C. | | | 40° C. | |
| Formulation No. | T0 | 1 W | 2 W | T0 | 1 W | 2 W |
| F1 (Ac 4.5) | 98.2 | 96.8 | 95.5 | 99.2 | 99.2 | 98.6 |
| F2 (Ac 5.0) | 98.2 | 96.8 | 95.6 | 99.3 | 99.3 | 98.7 |
| F3 (Ac 5.5) | 98.1 | 96.8 | 95.1 | 99.0 | 99.4 | 99.0 |
| F4 (Suc 5.0) | 98.3 | 96.7 | 95.7 | 99.3 | 99.1 | 98.4 |
| F5 (Suc 5.5) | 98.3 | 96.9 | 95.5 | 99.1 | 99.3 | 98.8 |
| F6 (Suc 6.0) | 98.4 | 96.4 | 94.6 | 99.3 | 99.3 | 98.9 |
| F7 (His 5.5) | 98.2 | 97.4 | 96.7 | 99.2 | 99.4 | 98.9 |
| F8 (His 6.0) | 98.2 | 97.7 | 96.7 | 99.3 | 99.3 | 98.9 |
| F9 (His 6.5) | 98.1 | 97.7 | 96.8 | 99.3 | 99.3 | 99.2 |
| F10 (PB 6.5) | 98.3 | 96.1 | 93.8 | 99.3 | 99.2 | 98.8 |
| F11 (PB 7.0) | 98.2 | 95.5 | 93.0 | 99.3 | 99.0 | 98.6 |
| F12 (PB 7.5) | 98.1 | 94.5 | 91.0 | 99.2 | 98.9 | 98.1 |

### DSC:

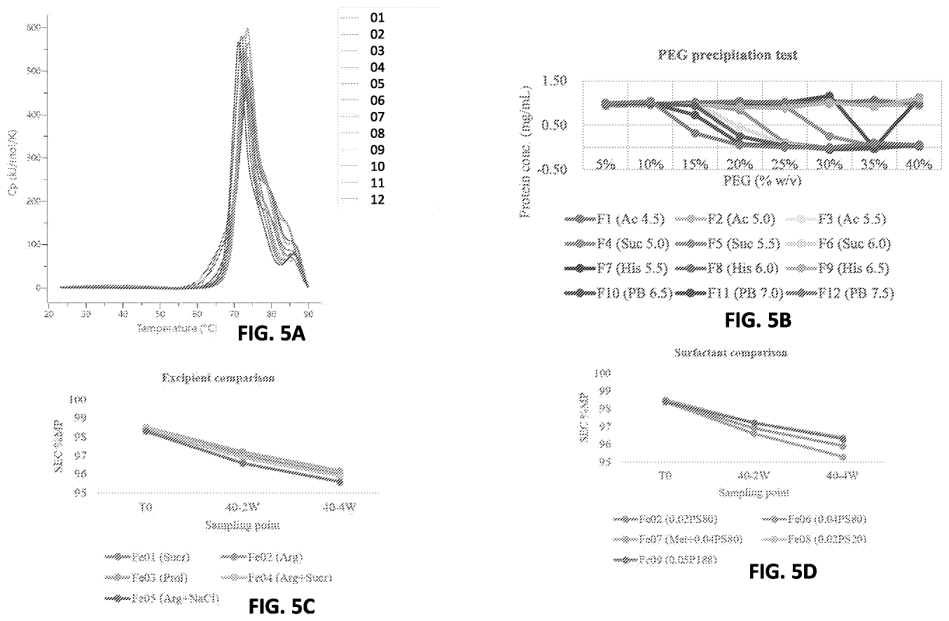

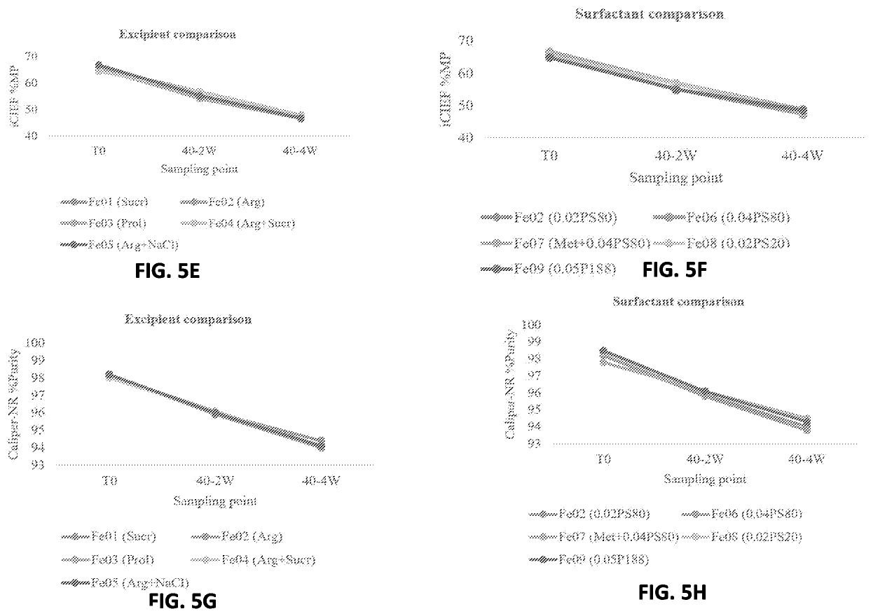

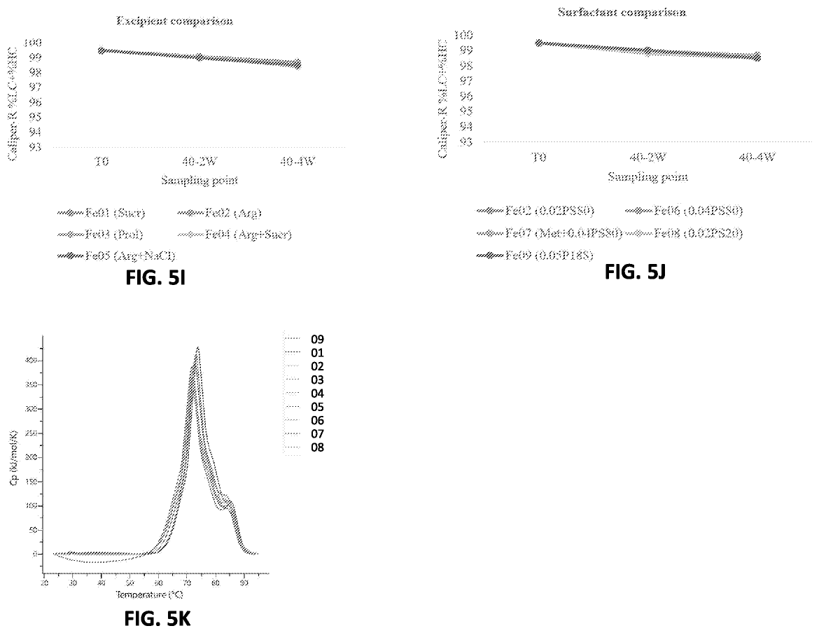

Table 41 and FIG. 5A show the data summary of DSC results for the pH/buffer study. All buffer systems showed Tm_{Onset} over 60° C. except for F1 (Ac 4.5) indicating good thermal stability overall. The Tm_{Onset} value increased with the increase of pH for the same buffer system except for PB buffer system.

TABLE 41
pH/buffer study: DSC

| Formulation | Tₒₙₛₑₜ | Tₘ₁ | Tₘ₂ | Tₘ₃ | Tₘ₄ |
|---|---|---|---|---|---|
| No. | (° C.) | (° C.) | (° C.) | (° C.) | (° C.) |
| F1 (Ac 4.5) | 58.8 | 66.2 | 72.8 | 79.0 | 84.6 |
| F2 (Ac 5.0) | 62.7 | 70.6 | 73.5 | 79.1 | 86.1 |
| F3 (Ac 5.5) | 65.1 | 72.5 | 73.6 | 78.6 | 86.4 |
| F4 (Suc 5.0) | 60.3 | 68.5 | 72.9 | 77.3 | 85.3 |
| F5 (Suc 5.5) | 63.5 | 70.9 | 72.8 | 78.9 | 86.0 |
| F6 (Suc 6.0) | 65.2 | 71.9 | 72.8 | 77.9 | 86.2 |
| F7 (His 5.5) | 60.2 | 67.5 | 72.9 | 78.0 | 84.3 |
| F8 (His 6.0) | 64.3 | 72.3 | 73.6 | 79.1 | 86.3 |
| F9 (His 6.5) | 66.1 | 71.2 | 73.4 | 78.1 | 86.7 |
| F10 (PB 6.5) | 65.8 | 70.3 | 72.5 | 76.8 | 86.2 |
| F11 (PB 7.0) | 65.3 | 69.1 | 71.7 | 75.9 | 86.0 |
| F12 (PB 7.5) | 64.7 | 68.1 | 70.9 | 75.2 | 85.8 |

### PEG Precipitation Test:

Table 42 and FIG. 5B show the data summary of PEG precipitation test results for the pH/buffer study. No substantial difference in solubility was observed for acetate and histidine buffer systems in this test. The protein conc. didn't show any decrease even with 40% of PEG in solution for these two buffer systems. The protein showed relatively worse solubility in succinate and phosphate buffer systems, and the solubility decreased with pH increased in the same buffer system.

TABLE 42
pH/buffer study: PEG precipitation

| | PEG (% w/v) | | | | | | | |
|---|---|---|---|---|---|---|---|---|
| Formulation | 5% | 10% | 15% | 20% | 25% | 30% | 35% | 40% |
| No. | Protein conc. (mg/mL) | | | | | | | |
| F1 (Ac 4.5) | 0.99 | 0.99 | 1.02 | 1.04 | 1.00 | 1.05 | 1.01 | 1.04 |
| F2 (Ac 5.0) | 0.93 | 0.97 | 0.97 | 0.98 | 0.90 | 1.00 | 0.96 | 0.93 |
| F3 (Ac 5.5) | 0.97 | 0.99 | 1.00 | 1.01 | 1.06 | 0.97 | 1.09 | 1.02 |
| F4 (Suc 5.0) | 0.93 | 0.95 | 0.98 | 0.98 | 0.93 | 0.25 | 0.03 | 0.06 |
| F5 (Suc 5.5) | 0.96 | 0.99 | 0.98 | 0.84 | 0.10 | −0.01 | 0.03 | 0.06 |
| F6 (Suc 6.0) | 0.97 | 1.00 | 1.00 | 0.46 | 0.07 | −0.05 | 0.04 | 0.04 |
| F7 (His 5.5) | 0.94 | 0.98 | 0.99 | 0.99 | 1.01 | 1.16 | 0.57* | 1.12 |
| F8 (His 6.0) | 0.96 | 0.99 | 1.01 | 1.03 | 1.02 | 1.00 | 1.06 | 0.98 |
| F9 (His 6.5) | 0.94 | 0.97 | 0.96 | 0.91 | 0.88 | 1.02 | 0.92 | 1.11 |
| F10 (PB 6.5) | 0.97 | 0.99 | 0.94 | 0.25 | 0.04 | −0.05 | −0.03 | 0.06 |
| F11 (PB 7.0) | 0.98 | 1.00 | 0.73 | 0.08 | 0.02 | 0.00 | 0.02 | 0.03 |
| F12 (PB 7.5) | 1.00 | 1.04 | 0.32 | 0.05 | 0.00 | −0.37* | 0.10 | 0.06 |

*Abnormal data points due to operation/test variation

In summary, the pH/buffer study investigated twelve pH/buffer systems under stress conditions (40° C. for up to 2 weeks), and the results showed that the stability of the anti-CD30L antibody was best in histidine buffer system, followed by acetate buffer system and succinate buffer system. The anti-CD30L antibody showed worst stability in PB buffer system. From the appearance results, not essentially free of visible particles (more than 5 particles) was observed in F9 (His 6.5), F10 (PB 6.5), F11 (PB 7.0) and F12 (PB 7.5) samples after incubation at 40° C. for 2 weeks. No substantial change was observed in pH or protein concentration in all buffer systems. From the PEG precipitation results, the protein had higher solubility in acetate and histidine buffer systems than that in succinate and phosphate buffer systems. Histidine buffer system performed better than all other tested buffer systems according to SE-UPLC, iCIEF and Caliper-SDS-NR results.

Taking all these results into consideration, F7 (His, pH 5.5) and F8 (His, pH 6.0) showed the best performance among all the pH/buffer types. 20 mM Histidine buffer under pH 5.5 was selected as the lead pH/buffer type considering the pI value of the anti-CD30L antibody (germlined clone 2), which is 8.36.

### 5.10.5 Excipients and Surfactant Strength Study

The excipients and surfactant strength study were to determine the optimal excipient that helps stabilize the protein. This study was conducted under different stress conditions (including 40° C. incubation, multiple freeze-thaw cycles and agitation).

The anti-CD30L antibody (germlined clone 2) was first concentrated to over 250 mg/mL. Stock solutions of 40% (w/w) sucrose, 1 M ArgHCl, 2 M proline, 1 M NaCl, 0.1 M methionine, 5% (w/w) polysorbate 80 (PS80), 5% (w/w) PS 20 and 10% (w/w) PS188 were used to prepare the formulations listed in Table 43. The amounts of the anti-CD30L antibody, excipient stock solutions and surfactant stock solutions were calculated, measured and well-mixed based on the formulation recipes. All formulation samples listed in Table 43 were eventually filtered through 0.22 μm PVDF filters, filled into 2 mL glass vials (1 mL per vial), then stoppered, capped and labeled immediately.

TABLE 43
Formulation candidates list of excipients
and surfactant strength study

| | Protein | | |
|---|---|---|---|
| Formulation | conc. | | |
| No. | (mg/mL) | Excipient(s) | Surfactant |
| Fe01 | 170 | 5% (w/v) sucrose | 0.02% (w/v) PS80 |
| Fe02 | | 100 mM ArgHCl | |
| Fe03 | | 220 mM proline | |
| Fe04 | | 50 mM ArgHCl, 2.5% | |
| | | (w/v) sucrose | |
| Fe05 | | 50 mM ArgHCl, 50 mM | |
| | | NaCl | |
| Fe06 | | 100 mM ArgHCl | 0.04% (w/v) PS80 |
| Fe07 | | 100 mM ArgHCl, 10 | 0.04% (w/v) PS80 |
| | | mM methionine | |
| Fe08 | | 100 mM ArgHCl | 0.02% (w/v) PS20 |
| Fe09 | | 100 mM ArgHCl | 0.05% (w/v) P188 |
| Fe10 | 200 | 5% (w/v) sucrose | 0.02% (w/v) PS80 |
| Fe11 | | 100 mM ArgHCl | |
| Fe12 | | 220 mM proline | |

Table 44 shows the sampling and testing for the excipients and surfactant strength study. For freeze and thaw (FT), samples were completely frozen at −70° C. and then thawed at room temperature (RT). For thermal stress, samples were stored at 40° C. for up to 4 weeks (W). For agitation stress, samples were agitated at 300 rpm for up to 3 days (D). Testing items including appearance, pH, protein conc., osmolality, viscosity, SE-UPLC, Caliper-SDS (NR & R), iCIEF, HIAC, DSC were performed in this study.

TABLE 44
Sampling and testing plan of excipients
and surfactant strength study

| Formulation | | | | Sampling Points | |
|---|---|---|---|---|---|
| No. | Attribute | Condition | T0 | and Assay | |
| Fe01-Fe12 | Thermal | 40° C. | X, Y, | 2 W | 4 W |
| | | | Z, W | X, Z | X, Z, W |
| | Agitation | 300 rpm, | | 1 D | 3 D |
| | | 25° C. | | X | X, Z |
| | FT | −70° C. | | 3 Cycles | 5 Cycles |
| | | to RT | | X | X, Z |

X = Appearance, pH, protein conc., SE-UPLC, Caliper-SDS (NR & R), iCIEF
Y = DSC, osmolality, Viscosity
Z = Sub-visible particle (HIAC)

Appearance, pH, protein conc., osmolality and viscosity:

Table 45 shows the results of appearance testing for the excipients and surfactant strength study. No substantial change in appearance was observed after 40° C. incubation for four weeks, freeze-thaw for 5 cycles (CYS) or agitation for 3 days.

TABLE 45
Excipients and surfactant strength study: appearance

| | Appearance | | | | | | |
|---|---|---|---|---|---|---|---|
| Formulation | | FT | | Agitation | | 40° C. | |
| No. | T0 | 3 CYS | 5 CYS | 1D | 3 D | 2 W | 4 W |
| Fe01 | SY, SO, | SY, SO, | SY, SO, | SY, SO, | SY, SO, | SY, SO, | SY, SO, |
| | FP | FP | FP | FP | FP | FP | FP |
| Fe02 | SY, SO, | SY, SO, | SY, SO, | SY, SO, | SY, SO, | SY, SO, | SY, SO, |
| | FP | FP | FP | FP | FP | FP | FP |
| Fe03 | SY, SO, | SY, SO, | SY, SO, | SY, SO, | SY, SO, | SY, SO, | SY, SO, |
| | FP | FP | FP | FP | FP | FP | FP |
| Fe04 | SY, SO, | SY, SO, | SY, SO, | SY, SO, | SY, SO, | SY, SO, | SY, SO, |
| | FP | FP | FP | FP | FP | FP | FP |
| Fe05 | SY, SO, | SY, SO, | SY, SO, | SY, SO, | SY, SO, | SY, SO, | SY, SO, |
| | FP | FP | FP | FP | FP | FP | FP |
| Fe06 | SY, SO, | SY, SO, | SY, SO, | SY, SO, | SY, SO, | SY, SO, | SY, SO, |
| | FP | FP | FP | FP | FP | FP | FP |
| Fe07 | SY, SO, | SY, SO, | SY, SO, | SY, SO, | SY, SO, | SY, SO, | SY, SO, |
| | FP | FP | FP | FP | FP | FP | FP |
| Fe08 | SY, SO, | SY, SO, | SY, SO, | SY, SO, | SY, SO, | SY, SO, | SY, SO, |
| | FP | FP | FP | FP | FP | FP | FP |
| Fe09 | SY, SO, | SY, SO, | SY, SO, | SY, SO, | SY, SO, | SY, SO, | SY, SO, |
| | FP | FP | FP | FP | FP | FP | FP |
| Fe10 | SY, SO, | SY, SO, | SY, SO, | SY, SO, | SY, SO, | SY, SO, | SY, SO, |
| | FP | FP | FP | FP | FP | FP | FP |
| Fe11 | SY, SO, | SY, SO, | SY, SO, | SY, SO, | SY, SO, | SY, SO, | SY, SO, |
| | FP | FP | FP | FP | FP | FP | FP |
| Fe12 | SY, SO, | SY, SO, | SY, SO, | SY, SO, | SY, SO, | SY, SO, | SY, SO, |
| | FP | FP | FP | FP | FP | FP | FP |

Abbreviation: SO (slightly opalescent); SY (slightly yellow); FP (free of visible particles); FT (freeze and thaw), CYS (cycle)

The results of osmolality, viscosity and protein concentrations in this study were shown in Table 46 and Table 47. The osmolality of all the formulations were between 269-398 mOsmol/kg, in the acceptable range for clinical use.

The viscosity results are summarized in Table 46. The viscosity was between 8.62-11.02 cP for Fe01-Fe09 with 170 mg/mL. Fe01 with only sucrose as excipient had the highest viscosity (11.02 cP) while Fe02 and Fe03 with only ArgHCl and proline as excipient had lower viscosity (˜9 cP), indicating that ArgHCl and proline could help reduce the viscosity. Fe04 with the excipient combination of sucrose and ArgHCl had the viscosity between Fe01 and Fe02, Fe05 with the excipient combination of ArgHCl and NaCl had similar viscosity as Fe02, different surfactant types and concentrations and addition of methionine (Fe02 and Fe06-Fe09) didn't cause much change on viscosity. Higher protein conc. for Fe10-Fe12 (200 mg/mL) also resulted in higher viscosity, which was between 17.40-22.21 cP. Even at the higher concentration (200 mg/ml), reduction of viscosity could also be seen for formulations with ArgHCl and proline, as compared to the formulation using sucrose as the excipient.

TABLE 46
Excipients and surfactant strength study: osmolality, viscosity and pH

| | | | pH | | | | | | |
|---|---|---|---|---|---|---|---|---|---|
| | Osmolality | Viscosity | | FT | | | | | |
| Formulation | (mOsm/kg) | (cp) | | 3 | 5 | Agitation | | 40° C. | |
| No. | T0 | | T0 | CYS | CYS | 1 D | 3 D | 2 W | 4 W |
| Fe01 | 299 | 11.02 | 5.3 | 5.4 | 5.4 | 5.4 | 5.4 | 5.3 | 5.3 |
| Fe02 | 272 | 8.98 | 5.4 | 5.4 | 5.4 | 5.4 | 5.4 | 5.4 | 5.4 |
| Fe03 | 349 | 8.99 | 5.4 | 5.4 | 5.4 | 5.4 | 5.4 | 5.4 | 5.4 |
| Fe04 | 283 | 9.72 | 5.4 | 5.4 | 5.4 | 5.4 | 5.4 | 5.4 | 5.4 |
| Fe05 | 287 | 9.04 | 5.4 | 5.4 | 5.4 | 5.4 | 5.4 | 5.4 | 5.4 |
| Fe06 | 269 | 8.62 | 5.4 | 5.4 | 5.4 | 5.4 | 5.4 | 5.4 | 5.4 |
| Fe07 | 283 | 9.02 | 5.4 | 5.4 | 5.4 | 5.4 | 5.4 | 5.4 | 5.4 |
| Fe08 | 273 | 8.78 | 5.4 | 5.4 | 5.4 | 5.4 | 5.4 | 5.4 | 5.4 |
| Fe09 | 269 | 9.12 | 5.4 | 5.4 | 5.4 | 5.4 | 5.4 | 5.4 | 5.4 |
| Fe10 | 342 | 22.21 | 5.3 | 5.4 | 5.4 | 5.4 | 5.3 | 5.4 | 5.4 |
| Fe11 | 303 | 18.09 | 5.4 | 5.4 | 5.4 | 5.4 | 5.4 | 5.4 | 5.4 |
| Fe12 | 398 | 17.40 | 5.4 | 5.4 | 5.4 | 5.4 | 5.4 | 5.4 | 5.4 |

TABLE 47
Excipients and surfactant strength study: Protein Conc.

| | Protein Conc. (mg/mL) | | | | | | |
|---|---|---|---|---|---|---|---|
| | | FT | | Agitation | | 40° C. | |
| Formulation No. | T0 | 3 CYS | 5 CYS | 1 D | 3 D | 2 W | 4 W |
| Fe01 | 180.1 | 176.6 | 177.5 | 175.3 | 176.4 | 175.7 | 178.0 |
| Fe02 | 183.0 | 173.1 | 178.6 | 173.6 | 181.6 | 175.4 | 176.9 |
| Fe03 | 179.8 | 173.8 | 179.9 | 173.0 | 179.4 | 177.7 | 176.6 |
| Fe04 | 183.6 | 175.1 | 181.6 | 174.4 | 183.5 | 179.8 | 179.1 |
| Fe05 | 178.9 | 174.1 | 180.0 | 179.8 | 180.2 | 176.6 | 176.6 |
| Fe06 | 180.8 | 176.7 | 181.2 | 175.4 | 181.1 | 178.7 | 178.1 |
| Fe07 | 181.6 | 176.0 | 174.8 | 175.8 | 185.8 | 178.3 | 178.2 |
| Fe08 | 178.9 | 177.1 | 175.7 | 174.5 | 181.2 | 179.3 | 176.3 |
| Fe09 | 180.9 | 178.3 | 177.0 | 173.4 | 181.3 | 180.8 | 176.7 |
| Fe10 | 217.2 | 212.3 | 222.9 | 209.8 | 220.5 | 216.1 | 211.9 |
| Fe11 | 216.9 | 208.4 | 212.5 | 205.6 | 221.3 | 212.0 | 207.1 |
| Fe12 | 212.9 | 202.3 | 209.7 | 203.8 | 215.3 | 211.3 | 210.4 |

*: samples were 10-fold diluted by corresponding buffer before UV280 test.

### Sub-visible Particle (HIAC):

Table 48 shows the results of sub-visible particle (HIAC) for the excipients and surfactant strength study.

TABLE 48
Excipients and surfactant strength
study: sub-visible particles (HIAC)

| | Sub-visible particles count (#/mL, ≥2 μm/≥10 μm/≥25 μm) | | | | |
|---|---|---|---|---|---|
| Formula- | | FT | Agitation | 40° C. | |
| tion No. | T0 | 5 CYS | 3 D | 2 W | 4 W |
| Fe01 | 202/51/2 | 422/63/2 | 186/18/0 | 433/45/3 | 346/38/1 |
| Fe02 | 26/3/0 | 161/3/0 | 377/6/0 | 175/3/0 | 89/4/0 |
| Fe03 | 47/3/0 | 329/3/0 | 190/3/0 | 69/4/0 | 116/3/0 |
| Fe04 | 60/1/0 | 554/6/0 | 400/6/0 | NA* | 264/6/0 |
| Fe05 | 23/3/0 | 667/14/1 | 1087/12/0 | 59/6/0 | 529/12/0 |
| Fe06 | 16/4/0 | 915/16/0 | 367/6/1 | 596/27/0 | 410/16/0 |
| Fe07 | 11/3/0 | 405/4/0 | 792/9/0 | 155/11/1 | 297/5/0 |
| Fe08 | 6/2/0 | 261/6/0 | 206/5/0 | 358/27/1 | 334/5/0 |
| Fe09 | 3/1/0 | 506/12/1 | 1402/9/0 | 343/13/0 | 655/13/0 |
| Fe10 | 46/1/0 | 280/6/0 | 228/8/1 | 172/11/0 | 156/3/0 |
| Fe11 | 8/1/0 | 211/5/0 | 266/18/0 | 223/12/1 | 543/12/0 |
| Fe12 | 10/2/1 | 228/6/0 | 129/9/0 | 109/3/0 | 228/5/0 |

*Test failed due to operation error

### SE-UPLC:

Table 49 and FIG. 5C-5D provide the data from the SE-UPLC testing for the excipients and surfactant strength study. Decrease of % MP (about 2.0-3.1%) was observed after 40° C. incubation for four weeks, which mainly came from increase in % HMW. No substantial change was observed for all formulation after freeze-thaw for 5 cycles or agitation for 3 days. Formulations containing PS20 (Fe08) or P188 (Fe09) performed better than PS80 (Fe02). Higher % HMW was observed in formulation containing higher PS80 concentration (Fe02 vs. Fe06), which could be mitigated by addition of methionine (Fe07).

TABLE 49
Excipients and surfactant strength study: SE-UPLC

| | SE-UPLC | | | | | | | | | |
|---|---|---|---|---|---|---|---|---|---|---|
| | Main peak (MP) (%) | | | | | | | High Molecular Weight (HMW) (%) | | |
| | | FT | | Agitation | | 40° C. | | | FT | |
| Formulation | | 3 | 5 | 1 | 3 | 2 | 4 | | 3 | 5 |
| No. | T0 | CYS | CYS | D | D | W | W | T0 | CYS | CYS |
| Fe01 | 98.5 | 98.4 | 98.4 | 98.4 | 98.3 | 97.0 | 96.0 | 1.5 | 1.5 | 1.5 |
| Fe02 | 98.4 | 98.4 | 98.3 | 98.4 | 98.3 | 96.9 | 95.9 | 1.5 | 1.5 | 1.5 |
| Fe03 | 98.5 | 98.4 | 98.4 | 98.5 | 98.3 | 97.2 | 96.2 | 1.5 | 1.5 | 1.5 |
| Fe04 | 98.5 | 98.4 | 98.4 | 98.5 | 98.3 | 96.9 | 95.9 | 1.5 | 1.5 | 1.5 |
| Fe05 | 98.3 | 98.3 | 98.1 | 98.3 | 98.2 | 96.6 | 95.6 | 1.6 | 1.7 | 1.7 |
| Fe06 | 98.4 | 98.4 | 98.3 | 98.4 | 98.3 | 96.6 | 95.3 | 1.5 | 1.5 | 1.5 |
| Fe07 | 98.5 | 98.5 | 98.3 | 98.5 | 98.3 | 97.2 | 96.3 | 1.5 | 1.5 | 1.5 |
| Fe08 | 98.4 | 98.4 | 98.3 | 98.4 | 98.3 | 97.2 | 96.4 | 1.5 | 1.6 | 1.5 |
| Fe09 | 98.4 | 98.4 | 98.3 | 98.5 | 98.2 | 97.2 | 96.3 | 1.5 | 1.5 | 1.5 |
| Fe10 | 98.4 | 98.4 | 98.3 | 98.3 | 98.1 | 96.7 | 95.6 | 1.6 | 1.6 | 1.6 |
| Fe11 | 98.3 | 98.3 | 98.2 | 98.4 | 98.1 | 96.6 | 95.5 | 1.6 | 1.6 | 1.7 |
| Fe12 | 98.4 | 98.4 | 98.3 | 98.3 | 98.1 | 96.9 | 95.8 | 1.5 | 1.6 | 1.6 |

| | SE-UPLC | | | | | | | | | | |
|---|---|---|---|---|---|---|---|---|---|---|---|
| | High Molecular Weight (HMW) (%) | | | | Low Molecular Weight (LMW) (%) | | | | | | |
| | Agitation | | 40° C. | | | FT | | Agitation | | 40° C. | |
| Formulation | 1 | 3 | 2 | 4 | | 3 | 5 | 1 | 3 | 2 | 4 |
| No. | D | D | W | W | T0 | CYS | CYS | D | D | W | W |
| Fe01 | 1.5 | 1.5 | 2.5 | 3.3 | 0.1 | 0.0 | 0.1 | 0.1 | 0.2 | 0.4 | 0.7 |
| Fe02 | 1.5 | 1.5 | 2.7 | 3.3 | 0.1 | 0.1 | 0.1 | 0.1 | 0.2 | 0.5 | 0.8 |
| Fe03 | 1.5 | 1.5 | 2.4 | 3.0 | 0.1 | 0.1 | 0.1 | 0.1 | 0.2 | 0.5 | 0.8 |
| Fe04 | 1.5 | 1.5 | 2.6 | 3.2 | 0.0 | 0.1 | 0.1 | 0.1 | 0.2 | 0.5 | 0.8 |
| Fe05 | 1.6 | 1.6 | 2.8 | 3.5 | 0.1 | 0.0 | 0.1 | 0.1 | 0.2 | 0.5 | 0.9 |
| Fe06 | 1.5 | 1.5 | 2.9 | 3.9 | 0.1 | 0.0 | 0.2 | 0.1 | 0.2 | 0.5 | 0.9 |
| Fe07 | 1.5 | 1.5 | 2.4 | 2.8 | 0.1 | 0.0 | 0.1 | 0.1 | 0.2 | 0.5 | 0.9 |
| Fe08 | 1.5 | 1.5 | 2.4 | 2.8 | 0.0 | 0.1 | 0.1 | 0.1 | 0.2 | 0.4 | 0.8 |
| Fe09 | 1.5 | 1.6 | 2.4 | 2.8 | 0.1 | 0.0 | 0.2 | 0.1 | 0.2 | 0.5 | 0.9 |
| Fe10 | 1.6 | 1.7 | 2.9 | 3.6 | 0.1 | 0.1 | 0.1 | 0.1 | 0.2 | 0.4 | 0.8 |
| Fe11 | 1.6 | 1.6 | 2.9 | 3.6 | 0.1 | 0.1 | 0.2 | 0.1 | 0.2 | 0.5 | 0.9 |
| Fe12 | 1.6 | 1.6 | 2.6 | 3.3 | 0.1 | 0.1 | 0.2 | 0.1 | 0.2 | 0.4 | 0.9 |

TABLE 50
Excipients and surfactant strength study: iCIEF

| | iCIEF | | | | | | | | | |
|---|---|---|---|---|---|---|---|---|---|---|
| | Main peak (MP) (%) | | | | | | | Acidic peaks (AP) (%) | | |
| | | FT | | Agitation | | 40° C. | | | FT | |
| Formulation | | 3 | 5 | 1 | 3 | 2 | 4 | | 3 | 5 |
| No. | T0 | CYS | CYS | D | D | W | W | T0 | CYS | CYS |
| Fe01 | 65.9 | 67.1 | 64.6 | 65.8 | 63.9 | 54.1 | 46.6 | 26.5 | 25.1 | 28.3 |
| Fe02 | 65.6 | 65.0 | 64.6 | 65.3 | 65.3 | 56.8 | 48.0 | 25.8 | 27.4 | 27.8 |
| Fe03 | 65.4 | 66.0 | 63.4 | 65.3 | 63.8 | 54.2 | 47.3 | 26.8 | 26.2 | 29.7 |
| Fe04 | 64.5 | 64.4 | 64.2 | 65.6 | 65.6 | 56.8 | 47.7 | 27.2 | 27.5 | 27.8 |
| Fe05 | 66.9 | 67.7 | 64.2 | 66.6 | 64.8 | 55.3 | 46.8 | 25.8 | 24.7 | 28.1 |
| Fe06 | 65.4 | 66.1 | 64.8 | 65.8 | 63.9 | 55.1 | 47.4 | 26.2 | 26.1 | 27.6 |
| Fe07 | 66.7 | 65.6 | 64.7 | 65.6 | 65.1 | 56.9 | 48.8 | 26.1 | 26.3 | 27.4 |
| Fe08 | 65.5 | 66.9 | 65.4 | 66.3 | 64.3 | 56.8 | 48.1 | 26.4 | 25.6 | 26.9 |
| Fe09 | 64.9 | 66.9 | 63.8 | 65.9 | 64.2 | 55.0 | 48.6 | 26.7 | 25.4 | 28.1 |
| Fe10 | 66.6 | 67.0 | 62.3 | 66.8 | 64.3 | 55.6 | 47.3 | 26.1 | 25.1 | 29.7 |
| Fe11 | 65.6 | 67.3 | 62.4 | 65.5 | 65.3 | 56.6 | 48.5 | 26.1 | 24.8 | 28.8 |
| Fe12 | 65.3 | 63.8 | 60.5 | 65.5 | 63.2 | 56.2 | 47.8 | 27.1 | 27.7 | 30.9 |

| | iCIEF | | | | | | | | | | |
|---|---|---|---|---|---|---|---|---|---|---|---|
| | Acidic peaks (AP) (%) | | | | Basic peaks (BP) (%) | | | | | | |
| | Agitation | | 40° C. | | | FT | | Agitation | | 40° C. | |
| Formulation | 1 | 3 | 2 | 4 | | 3 | 5 | 1 | 3 | 2 | 4 |
| No. | D | D | W | W | T0 | CYS | CYS | D | D | W | W |
| Fe01 | 26.7 | 28.7 | 37.2 | 46.2 | 7.7 | 7.8 | 7.1 | 7.5 | 7.4 | 8.7 | 7.3 |
| Fe02 | 26.6 | 26.9 | 34.9 | 44.2 | 8.6 | 7.6 | 7.6 | 8.1 | 7.9 | 8.3 | 7.8 |
| Fe03 | 27.5 | 28.7 | 37.7 | 46.5 | 7.8 | 7.8 | 7.0 | 7.2 | 7.6 | 8.1 | 6.2 |
| Fe04 | 26.8 | 27.2 | 35.0 | 45.2 | 8.2 | 8.1 | 8.0 | 7.7 | 7.3 | 8.2 | 7.2 |
| Fe05 | 26.0 | 27.6 | 36.9 | 44.9 | 7.3 | 7.6 | 7.8 | 7.4 | 7.6 | 7.8 | 8.4 |
| Fe06 | 26.3 | 27.5 | 36.5 | 44.7 | 8.4 | 7.8 | 7.6 | 7.9 | 8.7 | 8.4 | 8.0 |
| Fe07 | 26.3 | 27.3 | 34.4 | 43.1 | 7.2 | 8.1 | 7.8 | 8.1 | 7.6 | 8.8 | 8.1 |
| Fe08 | 25.9 | 27.8 | 34.1 | 43.7 | 8.1 | 7.5 | 7.7 | 7.9 | 7.9 | 9.1 | 8.2 |
| Fe09 | 25.6 | 28.1 | 36.3 | 42.9 | 8.5 | 7.8 | 8.1 | 8.5 | 7.6 | 8.7 | 8.6 |
| Fe10 | 25.5 | 28.5 | 35.8 | 45.2 | 7.4 | 7.9 | 8.0 | 7.7 | 7.2 | 8.6 | 7.5 |
| Fe11 | 25.5 | 27.4 | 34.5 | 43.5 | 8.3 | 7.9 | 8.8 | 9.0 | 7.3 | 8.9 | 8.0 |
| Fe12 | 26.6 | 29.3 | 35.7 | 44.4 | 7.6 | 8.5 | 8.6 | 7.9 | 7.5 | 8.1 | 7.8 |

TABLE 51
Excipients and surfactant strength study: Caliper-SDS (NR & R)

| | Caliper-SDS-NR (Purity %) | | | | | | | Caliper-SDS-R (LC + HC) % | | | | | | |
|---|---|---|---|---|---|---|---|---|---|---|---|---|---|---|
| | | FT | | Agitation | | 40° C. | | | FT | | Agitation | | 40° C. | |
| Formulation No. | T0 | 3 CYS | 5 CYS | 1 D | 3 D | 2 W | 4 W | T0 | 3 CYS | 5 CYS | 1 D | 3 D | 2 W | 4 W |
| Fe01 | 98.1 | 98.3 | 98.3 | 97.8 | 97.6 | 96.0 | 94.4 | 99.5 | 99.6 | 99.5 | 99.6 | 99.6 | 99.1 | 98.7 |
| Fe02 | 98.2 | 98.2 | 98.4 | 97.8 | 97.7 | 95.9 | 94.0 | 99.5 | 99.5 | 99.5 | 99.6 | 99.6 | 99.0 | 98.5 |
| Fe03 | 98.1 | 98.4 | 98.3 | 97.8 | 97.7 | 96.1 | 94.1 | 99.5 | 99.5 | 99.6 | 99.6 | 99.6 | 99.1 | 98.6 |
| Fe04 | 98.0 | 98.4 | 98.3 | 98.3 | 97.7 | 96.1 | 94.1 | 99.5 | 99.5 | 99.6 | 99.6 | 99.6 | 99.1 | 98.4 |
| Fe05 | 98.2 | 98.3 | 98.2 | 98.2 | 97.8 | 96.0 | 94.1 | 99.5 | 99.6 | 99.5 | 99.6 | 99.6 | 99.0 | 98.5 |
| Fe06 | 98.2 | 98.3 | 98.4 | 97.6 | 97.7 | 95.8 | 93.8 | 99.5 | 99.5 | 99.5 | 99.6 | 99.5 | 99.0 | 98.6 |
| Fe07 | 97.8 | 98.1 | 97.6 | 97.8 | 97.7 | 96.1 | 94.5 | 99.5 | 99.6 | 99.5 | 99.6 | 99.5 | 99.0 | 98.7 |
| Fe08 | 98.4 | 98.4 | 98.3 | 97.7 | 97.7 | 96.1 | 94.4 | 99.5 | 99.6 | 99.1 | 99.6 | 99.6 | 98.8 | 98.5 |
| Fe09 | 98.5 | 98.3 | 97.8 | 97.7 | 97.7 | 96.1 | 94.3 | 99.5 | 99.6 | 99.6 | 99.6 | 99.5 | 99.0 | 98.5 |
| Fe10 | 98.4 | 98.2 | 97.6 | 98.8* | 98.7* | 95.9 | 94.1 | 99.5 | 99.6 | 99.6 | 99.6 | 99.5 | 98.9 | 98.8 |
| Fe11 | 98.2 | 98.3 | 97.8 | 97.7 | 97.5 | 95.8 | 93.7 | 99.4 | 99.6 | 99.6 | 99.5 | 99.5 | 98.9 | 98.6 |
| Fe12 | 98.3 | 98.4 | 97.7 | 97.7 | 97.7 | 95.9 | 93.9 | 99.5 | 99.6 | 99.6 | 99.6 | 99.6 | 99.0 | 98.4 |

*Samples were retested.

iCIEF:

Table 50 and FIGS. 5E-5F show the results of iCIEF testing for the excipients and surfactant strength study. Decrease of % MP (about 16.4%-20.1%) was observed after 40° C. incubation for four weeks, which mainly came from increases in % AP. Slight decrease of % MP (3.2-4.8%) was observed in Fe10-Fe12 (200 mg/mL) after freeze-thaw for 5 cycles.

### Caliper-SDS (NR & R):

Table 51 and FIGS. 5G-5J show the data summary of Caliper-SDS (NR & R) testing results for the excipients and surfactant strength study. The results indicate that the Caliper-SDS purity remained stable in the formulation. When stored at 40° C. for 4 weeks, the purity for all formulations declined for 3.3-4.5% in the Caliper-SDS-NR, and declined for 0.7-1.1% in the Caliper-SDS-R. The results indicated that Caliper-SDS (NR & R) purity remain stable in the formulation.

### DSC:

Table 52 and FIG. 5K show the data summary of DSC results for the excipients and surfactant strength study. The Tmₒₙₛₑₜ values of all formulations were over 57° C. Formulations with sucrose (Fe01), sucrose and ArgHCl combination (Fe04) or L-proline (Fe03) had slightly higher Tmₒₙₛₑₜ value compared with formulation with ArgHCl (Fe02). Other formulations with ArgHCl (Fe05-Fe10) had similar Tm-onset value compared with Fe02.

TABLE 52
Excipients and surfactant strength study: DSC

| Formulation | Tmₒₙₛₑₜ | Tₘ₁ | Tₘ₂ | Tₘ₃ | Tₘ₄ |
|---|---|---|---|---|---|
| No. | (° C.) | (° C.) | (° C.) | (° C.) | (° C.) |
| Fe01 | 61.1 | 68.9 | 73.6 | 78.8 | 85.6 |
| Fe02 | 57.7 | 66.8 | 71.6 | 76.5 | 84.3 |
| Fe03 | 60.6 | 68.9 | 73.0 | 78.3 | 85.4 |
| Fe04 | 59.4 | 67.4 | 72.4 | 77.0 | 84.8 |
| Fe05 | 58.0 | 67.0 | 71.7 | 76.8 | 84.4 |
| Fe06 | 57.8 | 66.5 | 71.5 | 76.8 | 84.3 |
| Fe07 | 57.7 | 66.8 | 71.5 | 76.8 | 84.3 |
| Fe08 | 57.6 | 66.9 | 71.6 | 76.6 | 84.4 |
| Fe09 | 58.0 | 66.8 | 71.6 | 76.5 | 84.3 |

In the excipients and surfactant strength study, both ArgHCl and L-Proline could help reduce viscosity. ArgHCl was selected for further studies considering the totality of the data and practical factors.

The SE-UPLC results demonstrated PS20 (Fe08) and P188 (Fe09) performed better than PS80 (Fe02). Higher % HMW was observed for the formulation with higher PS80 content (Fe02 vs. Fe06), which could be mitigated by addition of methionine (Fe07). Thus, 0.02% (w/v) PS20 was selected considering the totality of the data and practical factors. The need for L-Methionine as an anti-oxidant was further tested in the following oxidation study in view of the potential surfactant degradation, hotspot in CDR and sterilization method with vaporized hydrogen peroxide.

Taken all these testing results and factors into consideration, 200 mg/mL protein, 20 mM histidine, 100 mM ArgHCl, 0.02% (w/v) PS20, at pH 5.5, and 200 mg/mL protein, 20 mM histidine, 100 mM ArgHCl, 10 mM L-Methionine, 0.02% (w/v) PS20, at pH 5.5 were further investigated in the subsequent oxidation studies.

### 5.10.6 Oxidation Study

Both H₂O₂ and tBHP are commonly used to induce Methionine oxidation, while tBHP was reported to also induce low level of Tryptophan oxidation in some molecules. The purpose of oxidation study is to evaluate the effect of H₂O₂, AAPH and tBHP oxidation on protein quality, and to determine whether L-Methionine should be added in the formulation. The anti-CD30L antibody (germlined clone 2) was first concentrated to over 250 mg/mL. Stock solutions of 1 M ArgHCl, 0.1 M methionine, 5% (w/w) PS 20, 10% (v/v) H₂O₂, 20% (v/v) tBHP and 25% (w/w) AAPH were prepared. The amounts of the anti-CD30L antibody, excipient stock solutions, surfactant stock solution and oxidant stock solutions were calculated, measured and well-mixed based on the formulation recipes. All formulation samples listed in Table 53 were eventually filtered through 0.22 μm PVDF filters, filled into 2 mL glass vials (1 mL per vial), then stoppered, capped and labeled immediately.

TABLE 53
Formulation candidates list of oxidation study

| | Protein | |
|---|---|---|
| Formula- | conc. | |
| tion No. | (mg/mL) | Formulation |
| Fo01 | 200 | 20 mM Histidine, 100 mM ArgHCl, 0.02% (w/v) |
| | | PS20, pH 5.5 |
| Fo02 | | 20 mM Histidine, 100 mM ArgHCl, 10 mM Met, |
| | | 0.02% (w/v) PS20, pH 5.5 |

Table 54 shows the sampling and testing for the oxidation study. Sample were treated with 0.1% H₂O₂, 0.2% tBHP, and 0.5% AAPH and stored separately at 40° C. for 24 hours. Testing items including appearance, pH, protein conc., SE-UPLC, Caliper (NR&R), iCIEF and PTM were performed in this study.

TABLE 54
Sampling and testing plan of oxidation study

| T0 | Attribute | 40° C. for 24 hours* |
|---|---|---|
| X, Y | Blank | X, (Y) |
| | 0.1% H2O2 | X, (Y) |
| | 0.2% tBHP | X, (Y) |
| | 0.5% AAPH | X, (Y) |

X = Appearance, pH, protein conc., SE-UPLC, iCIEF, Caliper-SDS (R & NR)
Y = PTM (optional based on other test results)
*The reaction will be terminated by freezing at −70° C.

### Appearance, pH and Protein Conc.:

Table 55 shows the data summary of appearance, pH and protein conc. for the oxidation study. Samples remained slightly yellow, slightly opalescent, and free of visible particles throughout the study. No substantial change in pH and protein conc. was observed.

TABLE 55
Oxidation study: appearance, protein conc., and pH

| | | | 24 h | | | |
|---|---|---|---|---|---|---|
| | Formulation | | | 0.1% | 0.2% | 0.5% |
| Items | No. | T0 | Blank | H₂O₂ | tBHP | AAPH |
| Appearance | Fo01 | SY, SO, FP | SY, SO, FP | SY, SO, FP | SY, SO, FP | SY, SO, FP |
| | Fo02 (Met) | SY, SO, FP | SY, SO, FP | SY, SO, FP | SY, SO, FP | SY, SO, FP |
| pH | Fo01 | 5.4 | 5.4 | 5.4 | 5.4 | 5.4 |
| | Fo02 (Met) | 5.4 | 5.4 | 5.4 | 5.4 | 5.4 |
| Conc. | Fo01 | 207.6 | 203.9 | 209.1 | 206.0 | 201.2 |
| | Fo02 (Met) | 207.7 | 205.1 | 199.9 | 205.4 | 203.8 |

SE-UPLC, iCIEF and Caliper (NR & R):

Table 56 provides the data summary from the SE-UPLC, iCIEF and Caliper-SDS (NR & R) testing results for the oxidation study. For 0.1% H₂O₂ treatment, SE-UPLC, iCIEF and Caliper-SDS (NR & R) results were comparable between Fo01 and Fo02 (Met). For 0.2% tBHP treatment, Fo01 performed slightly better than Fo02 (Met) based on the iCIEF results. For 0.5% AAPH treatment, Fo02 (Met) performed slightly better than Fo01 based on the SE-UPLC, iCIEF and Caliper-SDS-NR results.

TABLE 56
Oxidation study: SE-UPLC, iCIEF and Caliper-SDS (NR & R)

| | | | | 40° C.-24 h | | | |
|---|---|---|---|---|---|---|---|
| | | Formulation | | | 0.1% | 0.2% | 0.5% |
| Items | | No. | T0 | Blank | H₂O₂ | tBHP | AAPH |
| SE- | % MP/% HM | Fo01 | 98.2/1.6/ | 98.2/1.6/ | 95.7/3.8/ | 97.3/2.4/ | 92.9/6.7/ |
| UPLC | W/% LMW | | 0.2 | 0.3 | 0.4 | 0.3 | 0.5 |
| | | Fo02 (Met) | 98.2/1.6/ | 98.1/1.6/ | 95.8/3.8/ | 97.7/2.0/ | 94.9/4.6/ |
| | | | 0.2 | 0.3 | 0.4 | 0.3 | 0.5 |
| iCIEF | % MP/% AP/ | Fo01 | 64.4/8.0/ | 64.9/8.0/ | 57.4/7.2/ | 65.3/7.4/ | 59.3/7.5/ |
| | % BP | | 27.6 | 27.2 | 35.4 | 27.3 | 33.3 |
| | | Fo02 (Met) | 64.8/8.5/ | 64.1/9.0/ | 55.3/7.2/ | 59.7/9.1/ | 65.2/7.4/ |
| | | | 26.7 | 26.9 | 37.5 | 31.2 | 27.4 |
| Caliper-R | % LC + % HC | Fo01 | 99.5 | 99.5 | 99.5 | 99.5 | 98.8 |
| | | Fo02 (Met) | 99.6 | 99.5 | 99.5 | 99.5 | 99.0 |
| Caliper-NR | % Purity | Fo01 | 97.4 | 97.2 | 85.8 | 96.2 | 91.5 |
| | | Fo02 (Met) | 97.5 | 97.1 | 86.2 | 96.6 | 93.0 |

### Post Translational Modification (PTM):

Fo01 at T0, Fo01 and Fo02 (Met) under 0.1% H2O2 and 40° C. treatment for 24 hours was selected for PTM testing in view of potential sterilization with vaporized hydrogen peroxide. The major identified PTMs were N-glycosylation and the loss of C-terminal lysine on the heavy chain. In addition, low abundant modifications such as methionine oxidation, lysine hydroxylation, typtophan oxidation, proline amidation, following the loss of C-terminal lysine and glycine, asparagine deamidation and succinimide formation were also reported in the literature.

Under 0.1% H₂O₂ and 40° C. treatment for 24 hours, compared with sample Fo01 at T0, methione oxidation on HC: M34 in the CDR region, on HC: M83 and on LC: M4 slightly increased for samples Fo01 and Fo02 (Met). Methione oxidation on HC: M259 and on HC: M435 significantly increased for Fo01 and Fo02 (Met) samples. Other modifications were also reported with comparable ratios for samples Fo01 at T0, Fo01 and Fo02 (Met) under 0.1% H₂O₂ and 40° C. treatment for 24 hours.

In summary of the oxidation study, two formulations with or without L-Methionine under oxidation stress of three different oxidants (0.1% H₂O₂, 0.2% AAPH and 0.5% tBHP) were investigated in the oxidation study. No substantial change was observed in appearance, pH and protein conc. throughout the study. For 0.1% H₂O₂ treatment, SE-UPLC, iCIEF and Caliper-SDS (R & NR) results were comparable between Fo01 and Fo02 (Met). For 0.2% tBHP treatment, Fo01 performed slightly better than Fo02 (Met) based on the iCIEF results. For 0.5% AAPH treatment, Fo02 (Met) performed slightly better than Fo01 from the SE-UPLC, iCIEF and Caliper-SDS-NR results.

Overall, no substantial difference was found between Fo01 and Fo02 (Met) with 0.1% H₂O₂ treatment between two formulations, and vaporized hydrogen peroxide not AAPH was used for sterilization. Fo01 (200 mg/mL protein, 20 mM Histidine, 100 mM ArgHCl, 0.02% (w/v) PS20, pH 5.5) without the anti-oxidant methionine was selected as a representative exemplary formulation for the anti-CD30L antibody (germlined clone 2) considering the totality of the data and other considerations described herein.

### Summary of the Formulation Studies:

In the solubility study, formulations with 3 different protein conc. (100 mg/mL, 150 mg/mL and 200 mg/mL) under stress conditions (40° C. for up to 2 weeks) and 1 formulation with higher protein conc. (250 mg/mL) under long-term storage condition (2-8° C. for 24 hours) were investigated. Based on all the results in the solubility study, the stability and viscosity of this protein was acceptable at high concentration (e.g. ≥150 mg/ml)

In the pH/buffer screening study, 12 formulations designed from 4 buffer systems were evaluated under the thermal stress condition of 40° C. Based on the results obtained from appearance, pH, protein conc., SE-UPLC, Caliper-SDS (R & NR), iCIEF, DSC and PEG precipitation test, 20 mM histidine at pH 5.5 was selected for the excipients and surfactant screening study.

In the excipients and surfactant screening study, 12 designed formulations were evaluated in terms of their ability to maintain the anti-CD30L antibody stability under treatments of thermal stress condition at 40° C., freeze/thaw cycles, and agitation, as well as to reduce the viscosity. Based on the results obtained from appearance, pH, protein conc., SE-UPLC, Caliper-SDS (NR & R), iCIEF, HIAC, DSC, viscosity and osmolality, ArgHCl, methionine and PS20 were selected as excipients and surfactant used in the selected formulation. Therefore, two formulations, which were 200 mg/mL protein, 20 mM histidine, 100 mM ArgHCl, 0.02% (w/v) PS20, at pH 5.5, and 200 mg/mL protein, 20 mM histidine, 100 mM ArgHCl, 10 mM L-Methionine, 0.02% (w/v) PS20, at pH 5.5 were selected and were further evaluated in the subsequent oxidation study.

In the oxidation study, the two formulations with or without L-Methionine under oxidation stress of three different oxidants (0.1% H₂O₂, 0.2% AAPH and 0.5% tBHP) were investigated. In view of potential sterilization with vaporized hydrogen peroxide, comparison of Fo01 and Fo02 (Met) treated by 0.1% H₂O₂ was conducted. Since no substantial difference was observed with 0.1% H₂O₂ and 40° C. incubation for 24 hours' treatment between Fo01 and Fo02 (Met), Fo01 (200 mg/mL protein, 20 mM Histidine, 100 mM ArgHCl, 0.02% (w/v) PS20, pH 5.5) was selected as the formulation for the representative anti-CD30L antibody (germlined clone 2).

### 5.11 Example 11. Confirmation Studies of the Formulation Selected in Example 10 for the Representative Anti-CD30L Clone (Germlined Clone 2)

The disclosure further provides studies that confirm the formulation selected in Example 10 (Section 5.10) for the representative anti-CD30L clone (germlined clone 2) (referred to as the confirmation studies). To confirm reproducibility, a manufactured lot of the anti-CD30L antibody different from the 2 lots used in Example 10 (Section 5.10) was used in the confirmation studies.

Briefly, the selected formulation was confirmed through 3-month (M) stability study under various conditions (2-8° C., 25° C. and 40° C. incubation, agitation and incubation at 25° C. with the presence of H₂O₂). Inverted placement of the container with the formulation was tested for 2-8° C. and 25° C. Appearance, pH, protein conc., SE-UPLC, CE-SDS (NR&R), iCIEF, HIAC, viscosity, Tg′ and residual H₂O₂ testing were conducted as further described below.

### 5.11.1 Analytical Methods Used in the Confirmation Studies

Methods used in the confirmation studies to evaluate/determine the Appearance, Protein Concentration (A280), pH, and SE-UPLC have been described in Example 10 (Section 5.10).

### Liquid Particle Counter (HIAC):

A Liquid Particle Counting Systems (HIAC 9703+) was utilized to measure the size and counts of sub-visible particles in a bio-safety hood. To avoid introducing air bubbles and interference during examination, all samples were held in the bio-safety hood for at least 0.5 hour before testing. Each sample was tested for four consecutive runs, 0.45 mL each. The data in the first run was ignored, and the results were presented as average number of particles of ≥2 μm, ≥10 μm and ≥25 μm per mL (method conforms to USP <788> Particulate matter in injections).

iCIEF:
iCIEF is a purity analysis method used to monitor the percentage of charge variant species. The isoelectric point (pI) is an intrinsic property of a specific protein molecule and is the pH at which the protein molecule does not carry net electrical charge. Under an external electric field, the charge variants move along a continuous pH gradient formed by ampholytes and stop at where the pH equals its pI. The electropherogram is obtained at 280 nm. For WBP2454 protein, 20 μg sample was mixed with 80 μL master mix, which was composed of 0.5 μL of pI 7.05 marker, 0.5 μL of pI 9.91 marker, 1 μL of pharmalyte 3-10, 3 μL of pharmalyte 8-10.5, 35 μL of 1% methyl cellulose, 37.5 μL of 8 M urea solution, and 2.5 μL of ultrapure water. The mixture was then analyzed with the Maurice C. In the electropherogram, the peaks on the left of the main peak are acidic peaks, and the peaks on the right of the main peak are basic peaks. The pI value and relative abundance of the resolved peaks can be quantitated using chromatographic software (Empower 3).

### CE-SDS-NR & R:

Non-reduced: CE-SDS-NR, driven by a high-voltage direct current electric field, is a capillary electrophoresis method that allows protein separation by size. In this method, the reference standard or samples were diluted to 4 mg/mL with phosphate buffer-citric acid (PB-CA) buffer, and then 25 μL of diluted reference standard or sample was mixed with 75 μL of SDS buffer and 5 μL of 100 mM N-Ethylmaleimide (NEM). The mixed sample was incubated at 60° C. for 10 min. The Sample was then injected into the capillary filled with the separation gel buffer by the voltage at −5 kV for 20 seconds and separated in the capillary thermostatically controlled at 25° C. with voltage applied at −15 kV for 35 min, The UV spectrum at 220 nm was collected by the Photo Diode Array (PDA) detector and analyzed by normalizing the corrected peak areas. During the experiment, the auto sampler tray was maintained at 15° C.

Reduced: CE-SDS-R, driven by a high-voltage direct current electric field, is a capillary electrophoresis method that allows protein separation by size. 100 μg of protein was mixed with the dilution solution (PB-CA) to achieve a total volume of 25 μL. Then the solutions were heated in the presence of 75 μL of SDS buffer and 5 μL of 2-mercaptoethanol (BME) at 70° C. for 10 min. Then samples were injected into the capillary filled with the separation gel buffer by the voltage at −5 kV for 20 seconds. Separation in the capillary thermostatically was controlled at 25° C. with voltage applied at −15 kV for 30 min. The UV spectrum at 220 nm was collected by the PDA detector and analyzed by normalizing the corrected peak areas. During the experiment, the auto sampler tray was maintained at 15° C.

### Glass Transition Temperature (Tg′):

Tg′ is the glass transition temperature of the maximally freeze concentrated solute, and is a critical parameter in determining the state of frozen DS, below which the DS would be in glass state where potential physical or chemical change would be substantially limited. Tg′ of a formulation was measured by TA Q2000 DSC. 20 μL of the sample was sealed in an aluminum pan and transferred into the equipment. A sealed empty pan was used as the reference. The samples were cooled to −60° C. by a cooling rate of 10° C./minute (min), and kept at this temperature for 5 min, then were heated to 20° C. with heating rate of 10° C./min. The results were recorded and analyzed by Universal Analysis Software.

Residual Peroxide (H₂O₂):

Aqueous-compatible formulation quantitative peroxide kit (Thermo Scientific 23280) was used for peroxide quantification with the detection range between 0.1 ppm and 3.4 ppm. Mixture of Reagent A and Reagent B (volume ratio 1:100) was used as the working reagent. 20 μL of the sample, standard solutions with a series of concentrations, reference standard and blank sample were mixed well with 200 μL working reagent respectively, then incubated at room temperature for 15-20 min with protection from light. 2 μL of each prepared mixture was added to the Big Lunatic sample plate and the absorbance was measured with the wavelength at 595 nm. Plotting with molarity of standard solutions as x-axis and absorbance value as y-axis: y=A*x+B.

### Viscosity:

The viscosity of protein sample was measured by DHR-2 Hybrid Rheometer at 25° C. The sample volume for testing was 0.6 mL and only one test was performed. The scan shear rate ramped from 10-1500 l/s with data read per 10 decades. At least 5 continuous data points within a linear range were selected to calculate viscosity for Newtonian fluid.

### 5.11.2 Protocol and Results of the Confirmation Studies

A full-length anti-CD30L antibody (germlined clone 2) in the formulation of about 200 mg/ml anti-CD30L antibody, 20 mM histidine buffer, 100 mM arginine monohydrochloride, 0.02% (w/v) PS20, at pH 5.5 was used in this study. The samples were filtered with 0.22 μm Polyvinylidene Fluoride (PVDF) filter, filled into 8 mL glass vials (3.1 mL per vial), then stoppered, capped and labeled immediately.

Table 57 shows the sampling and testing in the confirmation study. Samples were stored separately at different temperatures including 2-8° C. (Inverted), 25° C. (Inverted) for up to 3 months, 40° C. (Upright) for up to 1 month and 25° C. (Upright) with the presence of 0.1-5.0 ppm H₂O₂ for up to 1 month. For agitation study, samples were agitated at 25° C. with a speed of 100 rpm for up to 3 days. Testing items including appearance, pH, protein conc., Tg′, viscosity, HIAC, SE-UPLC, CE-SDS-NR&R and iCIEF were performed in this study.

TABLE 57
Sampling and testing in the formulation confirmation study

| | | Sampling points and assay | | |
|---|---|---|---|---|
| Storage condition | T0 | 2 W | 1 M | 3 M |
| 2-8° C. (inverted) | X, Y, Q, | X | X, Y | X, Y |
| 25° C. (inverted) | | X | X, Y | X, Y |
| 40° C. (upright) | | X | X, Y | / |
| 25° C. (0.1/0.5/2.0/5.0 ppm | | X, W | X, Y, W | / |
| H₂O₂) (Upright) | | | | |

| | | |
|---|---|---|
| Agitation (100 rpm, 25° C.) | 1 D | 3 D |
| (upright) | X | X, Y |

X = Appearance, pH, protein conc., SE-UPLC, CE-SDS-NR & R, iCIEF
Y = Sub-visible particles (HIAC; W = Residual H₂O₂, Q = Tg′; viscosity

The testing results for samples (anti-CD30L antibody germlined clone 2, 20 mM histidine buffer, 100 mM arginine monohydrochloride, 0.02% (w/v) PS20, at pH 5.5) stored under different conditions are summarized in this Section 5.11.2. Tg′ is an informative parameter in determining the state of frozen anti-CD30L antibody, below which the antibody would be in glass state where potential physical or chemical change would be substantially limited. The measured value of Tg′ for the anti-CD30L antibody formulation was −21.93° C.

The viscosity for the anti-CD30L antibody formulation (about 200 mg/mL anti-CD30L antibody, 20 mM histidine buffer, 100 mM arginine monohydrochloride, 0.02% (w/v) PS20, pH 5.5) was 18.16 cP (25° C.).

5.11.2.1 Studies with Agitation

### Appearance, pH, Protein Conc., and Sub-Visible Particles (HIAC):

Table 58 shows the data summary of appearance, pH, protein conc., and sub-visible particles (HIAC) from the agitation study. Samples remained slightly yellow, slightly opalescent, and free of visible particles after agitation for 3 days (D). The amount of sub-visible particles (≥2 μm, ≥10 μm, and ≥25 μm) remained at a very low level. In addition, no substantial change in pH and the protein conc. was observed.

TABLE 58
Formulation confirmation study (agitation):
appearance, protein conc., pH, and HIAC

| | | | Protein conc. | | HIAC (particles/mL) | | |
|---|---|---|---|---|---|---|---|
| Sampling point | | Appearance | (mg/mL) | pH | ≥2 μm | ≥10 μm | ≥25 μm |
| T0 | | SY, SO, FP | 216.5 | 5.5 | 246 | 34 | 1 |
| Agitation | 1 D | | 212.5 | 5.6 | NT | NT | NT |
| | 3 D | | 208.6 | 5.5 | 498 | 77 | 0 |

Abbreviation: SY, slightly yellow; SO, slightly opalescent; FP, free of visible particles; NT, not tested.

### SE-UPLC:

Table 59 shows the data summary of the SE-UPLC testing for the agitation study. No substantial change was observed in the SE-UPLC monomer % after agitation for 3 days.

TABLE 59
Formulation confirmation study (agitation): SE-UPLC

SE-UPLC

| | | | |
|---|---|---|---|
| Sampling point | Monomer (%) | HMW (%) | LMW (%) |

| T0 | | 99.6 | 0.4 | ND |
|---|---|---|---|---|
| Agitation | 1 D | 99.3 | 0.4 | 0.3 |
| | 3 D | 99.3 | 0.5 | 0.2 |

Abbreviation: HMW, high molecular weight; LMW, low molecular weight; ND, not detected.

iCIEF:

Table 60 shows the data summary of iCIEF testing results from the agitation study. The measured Isoelectric Point (pI) of all samples was 8.8. No substantial change was observed in the iCIEF main peak % compared with T0 after agitation for 3 days.

TABLE 60
Formulation confirmation study (agitation): iCIEF

| | | iCIEF | | |
|---|---|---|---|---|
| Sampling point | | Main peak (%) | Acidic peaks (%) | Basic peaks (%) |
| T0 | | 67.8 | 23.7 | 8.6 |
| Agitation | 1 D | 67.8 | 23.5 | 8.7 |
| | 3 D | 67.7 | 23.6 | 8.7 |

### CE-SDS-NR & R:

Table 61 shows the data summary of the CE-SDS-NR & R testing from the agitation study. No substantial change in purity % was observed for the CE-SDS-NR & R testing after agitation for 3 days.

TABLE 61
Formulation confirmation study (agitation): CE-SDS-NR&R

| | CE-SDS-NR | CE-SDS-R |
|---|---|---|
| Sampling point | Purity % | (LC + HC) % |

| T0 | | | 97.8 | 98.7 |
|---|---|---|---|---|
| | Agitation | 1 D | 98.1 | 98.7 |
| | | 3 D | 98.2 | 98.7 |

Abbreviation: HC, heavy chain; LC, light chain.

In summary, all monitored quality attributes remained stable throughout the stability study after agitation for 3 days.

### 5.11.2.2 Stability Study at 2-8° C. (Inverted) Condition

### Appearance, pH, Protein Conc., and Sub-Visible Particles (HIAC):

Table 62A shows the data summary of appearance, pH, protein conc., and sub-visible particles (HIAC) from the stability study at 2-8° C. Samples remained slightly yellow, slightly opalescent, and free of visible particles after incubation at 2-8° C. for 3 months. The amount of sub-visible particles (≥2 μm, ≥10 μm, and ≥25 μm) remained at a very low level. In addition, no substantial change in pH and the protein concentration was observed.

TABLE 62A
Formulation confirmation study (2-8°
C.): appearance, protein conc., pH, and HIAC

| | | | Protein conc. | | HIAC (particles/mL) | | |
|---|---|---|---|---|---|---|---|
| Sampling point | | Appearance | (mg/mL) | pH | ≥2 μm | ≥10 μm | ≥25 μm |
| T0 | | SY, SO, FP | 216.5 | 5.5 | 246 | 34 | 1 |
| 2-8° C. | 2 W | | 216.9 | 5.5 | NT | NT | NT |
| | 1 M | | 218.3 | 5.6 | 211 | 66 | 1 |
| | 3 M | | 215.4 | 5.6 | 286 | 48 | 0 |

Abbreviation: SY, slightly yellow; SO, slightly opalescent; FP, free of visible particles; NT, not tested.

### SE-UPLC:

Table 62B shows the data summary of the SE-UPLC testing from the stability study at 2-8° C. No substantial change was observed in the SE-UPLC monomer % after stored at 2-8° C. for 3 months.

TABLE 62B
Formulation confirmation study (2-8° C.): SE-UPLC

| | | | SE-UPLC | | |
|---|---|---|---|---|---|
| | Sampling point | | Monomer (%) | HMW (%) | LMW (%) |
| T0 | | | 99.6 | 0.4 | ND |
| | 2-8° C. | 2 W | 99.5 | 0.5 | ND |
| | | 1 M | 99.5 | 0.5 | ND |
| | | 3 M | 99.4 | 0.6 | ND |

Abbreviation: HMW, high molecular weight; LMW, low molecular weight; ND, not detected.

iCIEF:

Table 63 shows the data summary of iCIEF testing results from the stability study at 2-8° C. The measured pI of all samples was 8.8. No substantial change was observed in iCIEF main peak % after stored at 2-8° C. for 3 months.

TABLE 63
Formulation confirmation study (2-8° C.): iCIEF

| | | iCIEF | | |
|---|---|---|---|---|
| Sampling point | | Main peak (%) | Acidic peaks (%) | Basic peaks (%) |
| T0 | | 67.8 | 23.7 | 8.6 |
| 2-8° C. | 2 W | 67.6 | 23.6 | 8.7 |
| | 1 M | 67.2 | 24.0 | 8.9 |
| | 3 M | 65.7 | 25.3 | 9.0 |

### CE-SDS-NR & R:

Table 64 shows the data summary of the CE-SDS-NR & R testing from the stability study at 2-8° C. No substantial change in purity % was observed for the CE-SDS-NR & R testing when stored at 2-8° C. for 3 months.

TABLE 64
Formulation confirmation study (2-8° C.): CE-SDS-NR & R

| | CE-SDS-NR | CE-SDS-R |
|---|---|---|
| Sampling point | Purity % | (LC + HC) % |

| T0 | | | 97.8 | 98.7 |
|---|---|---|---|---|
| | 2-8° C. | 2 W | 98.1 | 98.7 |
| | | 1 M | 98.1 | 98.8 |
| | | 3 M | 98.0 | 98.4 |

Abbreviation: HC, heavy chain; LC, light chain.

In summary, all monitored quality attributes remained stable throughout the stability study at 2-8° C. (Inverted) condition for up to 3 months.

### 5.11.2.3 Stability Study at 25° C. (Inverted) Condition

### Appearance, pH, Protein Conc., and Sub-Visible Particles (HIAC):

Table 65 shows the data summary of appearance, pH, protein conc., and sub-visible particles (HIAC) from the stability study at 25° C. Samples remained slightly yellow, slightly opalescent, and free of visible particles after incubation at 25° C. for 3 months. The amount of sub-visible particles (≥2 μm, ≥10 μm, and ≥25 μm) remained at a very low level. In addition, no substantial change in pH and the protein concentration was observed.

TABLE 65
Formulation confirmation study (25°
C.): appearance, protein conc., pH, and HIAC

| | | | Protein conc. | | HIAC (particles/mL) | | |
|---|---|---|---|---|---|---|---|
| Sampling point | | Appearance | (mg/mL) | pH | ≥2 μm | ≥10 μm | ≥25 μm |
| T0 | | SY, SO, FP | 216.5 | 5.5 | 246 | 34 | 1 |
| 25° C. | 2 W | | 215.2 | 5.5 | NT | NT | NT |
| | 1 M | | 214.3 | 5.6 | 115 | 2 | 0 |
| | 3 M | | 215.3 | 5.5 | 93 | 0 | 0 |

Abbreviation: SY, slightly yellow; SO, slightly opalescent; FP, free of visible particles; ND, not tested.

### SE-UPLC:

Table 66 shows the data summary of the SE-UPLC testing from the stability study at 25° C. Slight decrease (1.1%) was observed in the monomer % after storage at 25° C. for 3 months.

TABLE 66
Formulation confirmation (25° C.): SE-UPLC

SE-UPLC

| | | | |
|---|---|---|---|
| Sampling point | Monomer (%) | HMW (%) | LMW (%) |

| T0 | | 99.6 | 0.4 | ND |
|---|---|---|---|---|
| 25° C. | 2 W | 99.0 | 0.6 | 0.3 |
| | 1 M | 99.0 | 0.8 | 0.2 |
| | 3 M | 98.5 | 1.1 | 0.4 |

Abbreviation: HMW, high molecular weight; LMW, low molecular weight; ND, not detected.

iCIEF:

Table 67 shows the data summary of iCIEF testing results from the stability study at 25° C. The measured pI of all samples was 8.8. The main peak % decreased by 6.4% with increase of 5.1% in the acidic peaks % compared with T0 after stored at 25° C. for 3 months.

TABLE 67
Formulation confirmation (25° C.): iCIEF

iCIEF

| | Main | Acidic | Basic |
|---|---|---|---|
| Sampling point | peak (%) | peaks (%) | peaks (%) |

| T0 | | 67.8 | 23.7 | 8.6 |
|---|---|---|---|---|
| 25° C. | 2 W | 66.6 | 24.5 | 8.9 |
| | 1 M | 65.4 | 25.1 | 9.5 |
| | 3 M | 61.4 | 28.8 | 9.8 |

### CE-SDS-NR & R:

Table 68 shows the data summary of the CE-SDS-NR & R testing from the stability study at 25° C. No obvious change in purity % (0.6-0.7%) was observed for the CE-SDS-NR & R testing after stored at 25° C. for 3 months.

TABLE 68
Formulation confirmation study (25° C.): CE-SDS-NR & R

| | | CE-SDS-NR | CE-SDS-R |
|---|---|---|---|
| Sampling point | | Purity % | (LC + HC) % |
| T0 | | 97.8 | 98.7 |
| 25° C. | 2 W | 97.9 | 98.7 |
| | 4 W | 98.1 | 98.6 |
| | 3 M | 97.1 | 98.1 |

Abbreviation: HC, heavy chain; LC, light chain.

In summary, all monitored quality attributes remained relatively stable (changes within pharmaceutically acceptable range) throughout the stability study at 25° C. (Inverted) condition for up to 3 months. The decrease rate of monomer % in SE-UPLC and main peak % in iCIEF was in the pharmaceutically acceptable range.

### 5.11.2.4 Stability Study at 40° C. (Upright) Condition

### Appearance, pH, Protein Conc., and Sub-Visible Particles (HIAC):

Table 69 shows the data summary of appearance, pH, protein conc., and sub-visible particles (HIAC) from the stability study at 40° C. Samples remained slightly yellow, slightly opalescent, and free of visible particles after incubation at 40° C. for up to 1 month. The amount of sub-visible particles (≥2 μm, ≥10 μm, and ≥25 μm) remained at a very low level. In addition, no substantial change in pH and the protein conc. was observed.

TABLE 69
Formulation confirmation study (40°
C.): appearance, protein conc., pH, and HIAC

| | | | Protein conc. | | HIAC (particles/mL) | | |
|---|---|---|---|---|---|---|---|
| Sampling point | | Appearance | (mg/mL) | pH | ≥2 μm | ≥10 μm | ≥25 μm |
| T0 | | SY, SO, FP | 216.5 | 5.5 | 246 | 34 | 1 |
| 40° C. | 2 W | | 214.5 | 5.5 | NT | NT | NT |
| | 1 M | | 215.7 | 5.5 | 330 | 1 | 0 |

Abbreviation: SY, slightly yellow; SO, slightly opalescent; FP, free of visible particles; NT, not tested.

### SE-UPLC:

Table 70 shows the data summary of the SE-UPLC testing from the stability study at 40° C. After incubation at 40° C. for 1 month, a slight decline of 2.2% in monomer % was observed together with a slight increase of 1.4% in the HMW % species and a slight increase of 0.8% in the LMW % species.

TABLE 70
Formulation confirmation (40° C.): SE-UPLC

SE-UPLC

| | | | |
|---|---|---|---|
| Sampling point | Monomer (%) | HMW (%) | LMW (%) |

| T0 | | 99.6 | 0.4 | ND |
|---|---|---|---|---|
| 40° C. | 2 W | 97.9 | 1.2 | 0.9 |
| | 1 M | 97.4 | 1.8 | 0.8 |

Abbreviation: HMW, high molecular weight; LMW, low molecular weight; ND, not detected.

iCIEF:

Table 71 shows the data summary of iCIEF testing results from the stability study at 40° C. The measured pI of all samples was 8.8. After incubation at 40° C. for 1 month, the main peak % decreased by 18.2% with increase of 16.1% in the acidic peaks %.

TABLE 71
Formulation confirmation (40° C.): iCIEF

iCIEF

| | Main | Acidic | Basic |
|---|---|---|---|
| Sampling point | peak (%) | peaks (%) | peaks (%) |

| T0 | | 67.8 | 23.7 | 8.6 |
|---|---|---|---|---|
| 40° C. | 2 W | 58.3 | 31.9 | 9.7 |
| | 1 M | 49.6 | 39.8 | 10.5 |

### CE-SDS-NR & R

Table 72 shows the data summary of the CE-SDS-NR & R testing from the stability study at 40° C. After incubation at 40° C. for 1 month, the CE purity % of this formulation decreased by 2.6% in the NR evaluation and 1.4% in the R evaluation.

TABLE 72
Formulation confirmation study (40° C.): CE-SDS-NR & R

| | | CE-SDS-NR | CE-SDS-R |
|---|---|---|---|
| Sampling point | | Purity % | (LC + HC) % |
| T0 | | 97.8 | 98.7 |
| 40° C. | 2 W | 96.6 | 97.7 |
| | 1 M | 95.2 | 97.3 |

Abbreviation: HC, heavy chain; LC, light chain.

In summary, all monitored quality attributes remained relatively stable (changes within pharmaceutically acceptable range) throughout the stability study at 40° C. (Upright) condition for up to 1 month. The decrease rate in SE-UPLC monomer %, CE-SDS-R & NR purity % and iCIEF main peak % was in the pharmaceutically acceptable range.

5.11.2.5 Stability Study with 0.1-5.0 ppm H₂O₂ Treatment at 25° C. (Upright) Condition
Appearance, pH, Protein Conc., and Sub-Visible Particles (HIAC), Residual H₂O₂:

Table 73 shows the data summary of appearance, pH, protein conc., sub-visible particles (HIAC) and residual H₂O₂ from the stability study with 0.1-5.0 ppm H₂O₂ treatment at 25° C. Samples remained slightly yellow, slightly opalescent, and free of visible particles after incubation at 25° C. with 0.1, 0.5, 2.0 and 5.0 ppm H₂O₂ treatment for 1 month, respectively. The amount of sub-visible particles (≥2 μm, ≥10 μm, and ≥25 μm) remained at a very low level for all samples after 1-month incubation. Residual H₂O₂ was not detected after 1-month incubation for all samples. In addition, no substantial change in pH and the protein concentration was observed.

TABLE 73
Formulation confirmation study (0.1-5.0 ppm H₂O₂ treatment at
25° C.): appearance, protein conc., pH, HIAC and residual H₂O₂

| | | | Protein | | | | | Residual |
|---|---|---|---|---|---|---|---|---|
| | | | conc. | | HIAC (particles/mL) | | | H₂O₂ |
| Sampling point | | Appearance | (mg/mL) | pH | ≥2 μm | ≥10 μm | ≥25 μm | (ppm) |
| T0 | | SY, SO, FP | 216.5 | 5.5 | 246 | 34 | 1 | NT |
| 25° C. + 0.1 | 2 W | | 210.9 | 5.5 | NT | NT | NT | 0.1 |
| ppm H₂O₂ | 1 M | | 214.0 | 5.5 | 17 | 2 | 0 | ND |
| 25° C. + 0.5 | 2 W | | 215.1 | 5.5 | NT | NT | NT | ND |
| ppm H₂O₂ | 1 M | | 215.3 | 5.5 | 25 | 0 | 0 | ND |
| 25° C. + 2.0 | 2 W | | 210.2 | 5.5 | NT | NT | NT | 0.1 |
| ppm H₂O₂ | 1 M | | 212.9 | 5.5 | 26 | 0 | 0 | ND |
| 25° C. + 5.0 | 2 W | | 209.5 | 5.5 | NT | NT | NT | ND |
| ppm H₂O₂ | 1 M | | 211.6 | 5.5 | 26 | 5 | 1 | ND |

Abbreviation: SY, slightly yellow; SO, slightly opalescent; FP, free of visible particles; NT, not tested; ND, not detected.

### SE-UPLC:

Table 74 shows the data summary of the SE-UPLC testing from the stability study with 0.1-5.0 ppm H₂O₂ treatment at 25° C. Slight decrease (0.6%) was observed in the monomer % after being stored at 25° C. for 1 month with 0.1-5.0 ppm H₂O₂ treatment. No clear correlation between H₂O₂ concentration and monomer % decline was observed. The decrease could be ascribed to 25° C. incubation rather than H₂O₂ treatment, for the similar decrease in monomer % compared with samples without H₂O₂ treatment (refer to 5.4.3).

TABLE 74
Formulation confirmation (0.1-5.0 ppm
H₂O₂ treatment at 25° C.): SE-UPLC

| | | SE-UPLC | | |
|---|---|---|---|---|
| Sampling point | | Monomer (%) | HMW (%) | LMW (%) |
| T0 | | 99.6 | 0.4 | ND |
| 25° C. + 0.1 ppm | 2 W | 99.1 | 0.7 | 0.3 |
| H₂O₂ | 1 M | 99.0 | 0.8 | 0.2 |
| 25° C. + 0.5 ppm | 2 W | 99.1 | 0.6 | 0.3 |
| H₂O₂ | 1 M | 99.0 | 0.8 | 0.2 |
| 25° C. + 2.0 ppm | 2 W | 99.1 | 0.6 | 0.3 |
| H₂O₂ | 1 M | 99.0 | 0.9 | 0.2 |
| 25° C. + 5.0 ppm | 2 W | 99.1 | 0.7 | 0.3 |
| H₂O₂ | 1 M | 99.0 | 0.9 | 0.2 |

Abbreviation: HMW, high molecular weight; LMW, low molecular weight, ND, not detected.

iCIEF:

Table 75 shows the data summary of iCIEF testing results from the stability study with 0.1-5.0 ppm H₂O₂ treatment at 25° C. The measured pI of all samples was 8.8. No obvious change in iCIEF main peak % was observed after stored at 25° C. for 1 month with 0.1-5.0 ppm H₂O₂ treatment.

TABLE 75
Formulation confirmation (0.1-5.0 ppm
H₂O₂ treatment at 25° C.): iCIEF

| | | iCIEF | | |
|---|---|---|---|---|
| | | Main | Acidic | Basic |
| Sampling point | | peak (%) | peaks (%) | peaks (%) |
| T0 | | 67.8 | 23.7 | 8.6 |
| 25° C. + 0.1 ppm | 2 W | 67.0 | 24.3 | 8.8 |
| H₂O₂ | 1 M | 65.2 | 25.3 | 9.5 |
| 25° C. + 0.5 ppm | 2 W | 66.0 | 25.2 | 8.8 |
| H₂O₂ | 1 M | 64.8 | 26.0 | 9.1 |
| 25° C. + 2.0 ppm | 2 W | 65.7 | 25.3 | 9.0 |
| H₂O₂ | 1 M | 65.1 | 25.3 | 9.7 |
| 25° C. + 5.0 ppm | 2 W | 66.4 | 24.5 | 9.1 |
| H₂O₂ | 1 M | 65.3 | 25.4 | 9.3 |

### CE-SDS-NR & R

Table 76 shows the data summary of the CE-SDS-NR & R testing from the stability study with 0.1-5.0 ppm H₂O₂ treatment at 25° C. No obvious change was observed in CE-SDS-NR & R purity % after stored at 25° C. for 1 month with 0.1 to 5.0 ppm H₂O₂ treatment.

TABLE 76
Formulation confirmation study (0.1-5.0 ppm
H₂O₂ treatment at 25° C.): CE-SDS-NR & R

| | CE-SDS-NR | CE-SDS-R |
|---|---|---|
| Sampling point | Purity % | (LC + HC) % |

| T0 | | | 97.8 | 98.7 |
|---|---|---|---|---|
| | 25° C. + 0.1 ppm | 2 W | 98.0 | 98.7 |
| | H₂O₂ | 1 M | 97.8 | 98.6 |
| | 25° C. + 0.5 ppm | 2 W | 97.9 | 98.7 |
| | H₂O₂ | 1 M | 97.9 | 98.6 |
| | 25° C. + 2.0 ppm | 2 W | 97.8 | 98.7 |
| | H₂O₂ | 1 M | 97.7 | 98.6 |
| | 25° C. + 5.0 ppm | 2 W | 97.6 | 98.8 |
| | H₂O₂ | 1 M | 97.8 | 98.5 |

Abbreviation: HC, heavy chain; LC, light chain.

In summary, all monitored quality attributes remained relatively stable (changes within pharmaceutically acceptable range) throughout the stability study with 0.1-5.0 ppm H₂O₂ treatment at 25° C. (Upright) for up to 1 month. The decrease of SE-UPLC monomer % could be ascribed to 25° C. incubation, rather than H₂O₂ treatment. Residual H₂O₂ was not detected after 1-month incubation at 25° C., which suggesting that H₂O₂ has been degraded completely after 25° C.-1 M incubation.

Taking all the confirmation studies together, the testing results indicated no obvious effect on the stability of anti-CD30L antibody as formulated after agitation for 3 days and 5° C. (Inverted) incubation for 3 months.

Slight decline in SE-UPLC monomer % (1.1%) and iCIEF main peak % (6.4%) was observed after 25° C. (Inverted) incubation for 3 months. Decline in SE-UPLC monomer % (2.2%), CE-SDS-NR purity % (2.6%), CE-SDS-R purity % (1.4%) and iCIEF main peak % (18.2%) was observed after incubation at 40° C. (Upright) for 1 month. These declines were within the pharmaceutically acceptable range.

Slight decrease in SE-UPLC (0.6%) was observed after 25° C. incubation for 1 month with 0.1-5.0 ppm H₂O₂ treatment and no clear correlation between H₂O₂ concentration and monomer % decline was observed, which could be ascribed to 25° C.-1 M incubation rather than H₂O₂ treatment, indicating that the addition of H₂O₂ for less than 5 ppm showed almost no impact on protein quality.

From the perspective of container closure system (CCS) evaluation, good stability was observed in inverted samples incubated at 5° C. and 25° C. with fully contact with the stopper and vial for up to 3 months, which indicates compatibility between the anti-CD30L antibody and the CCS.

Overall, this study supported the stability of 20 mM histidine buffer, 100 mM arginine monohydrochloride, 0.02% (w/v) PS20, at pH 5.5 as the formulation for anti-CD30L antibody for a range of concentrations, including high antibody concentrations, e.g. at or above about 200 mg/ml. The compatible container closure systems include 8 R vial (Schott-1531541), 20 mm stopper (West-1970-0376) and 20 mm cap (West-5420-2960).

### 5.12 Example 12. Structure of anti-CD30L Antibody and CD30L Complex and Epitope Mapping Based on the Structure and XL-MS Analysis

This study was conducted to collect epitope and paratope information on the CD30L/Anti-CD30L antibody complex by (i) obtaining a high resolution structure of the complex utilizing cryogenic electron microscopy (cryo-EM) and (ii) confirming the epitope and paratope with the XL-MS cross-linking experiments (which has been described in detail in Section 5.7, Example 7).

### 5.12.1 Cryo-EM Sample Preparation and Data Acquisition

The purified Fab fragment of a representative anti-CD30L antibody (germlined clone 2) was prepared by FabALACTICA IgdE cysteine protease digestion and Fc resin spin column purification (Genovis). The CD30L/germlined-clone-2-Fab complex was prepared for CryoEM analysis by solubilizing lyophilized CD30L protein with a solution of germlined-clone-2 Fab. Uncomplexed Fab was removed from sample by size exclusion chromatography fractionation and complex was concentrated to 4.0 mg/mL using a 10,000 molecular weight cutoff Amicon spin concentrator. The complex was used to prepare CryoEM grids the same day, without freezing of the sample.

Purified CD30L: germlined-clone-2-Fab complex sample was diluted to 2.0 mg/mL in a buffer containing 25 mM HEPES pH 7.4, 150 mM NaCl, and 0.035% (w/v) fluorinated octyl maltoside immediately prior to vitrification. Blotting and vitrification was performed using a Vitrobot Mk IV (ThermoFisher Scientific, Inc.), onto a freshly glow-discharged Quantifoil R1.2/1.3 300 Au mesh grid (Quantifoil Micro Tools GmbH). Exposure movies were collected using a Titan Krios G3i electron microscope (ThermoFisher Scientific, Inc.) equipped with a Gatan BioQuantum K3 energy filter and camera (Gatan, Inc.). Microscope and camera parameters are given in Table 76.

TABLE 76
Electron Microscopy Parameters

| | |
|---|---|
| Accelerating voltage | 300 kV |
| Nominal magnification | 81,000x |
| Camera mode | Counted super-resolution + CDS |
| Calibrated pixel size | 0.534 Å (super-resolution)/ |
| | 1.068 Å (physical) |
| Dose rate | 8 e⁻/physical pixel/s |
| Total exposure dose | 60 e⁻/Å² |
| Movie frames | 52 |
| Energy filter width | 20 eV |

### 5.12.2 Cryo-EM Image Processing

Data processing was carried out using the Relion v4.0-beta2 software package unless otherwise noted. During motion correction, movie data were dose-weighted and 2× binned back to the camera physical pixel size. CTF parameters for each micrograph sum were fit out using CTFFind v4.1.14 on movie frame power spectra sums. Removal of micrographs with extremely high fitted total motion, poor power spectra Thon rings or CTF estimation fits, or large crystalline ice regions resulted in 3,817 micrographs used for downstream data processing. Potential particles were picked using the Topaz particle picker, using a model that was trained on a prior pilot dataset collected under identical conditions. This pilot data was also used to generate an ab-initio initial 3D reference. 258,298 particles remained after 2D classification and coarsely-sampled 3D classification. These were subject to rounds of 3D auto-refinement, CTF parameter refinement, per-particle motion correction, and fixed-pose 3D classification until the resolution of the reconstruction failed to improve. 163,805 particles contributed to the final 3D refinement.

### 5.12.3 Atomic Model Building

The atomic model was built starting from the publicly available AlphaFold prediction for CD30L and the germlined-clone-2 Fab model created with Antibody Modeler in Molecular Operating Environment (MOE) software, v.2020.0901, docked into the experimental map using Chimera. The model was refined by iterating between manual adjustment using Coot and automated real-space refinement in Phenix.

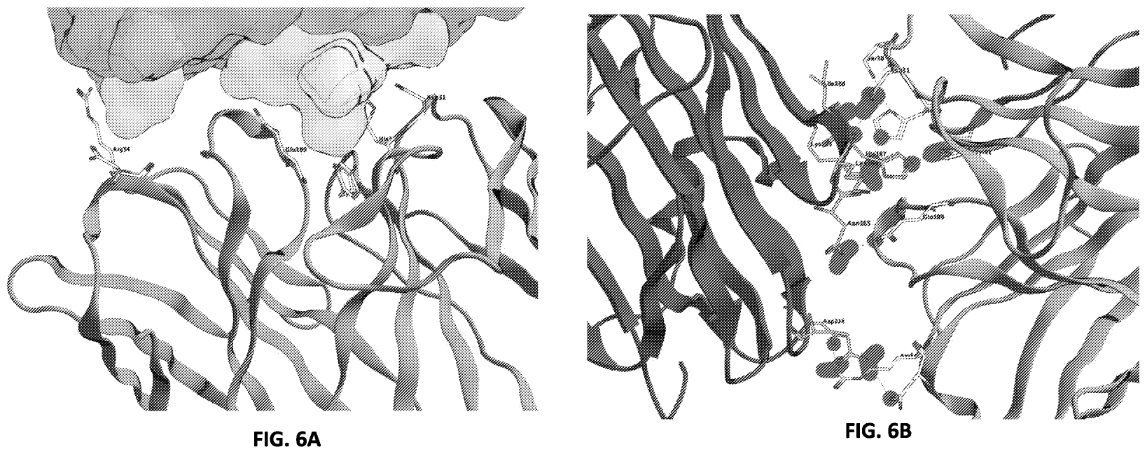

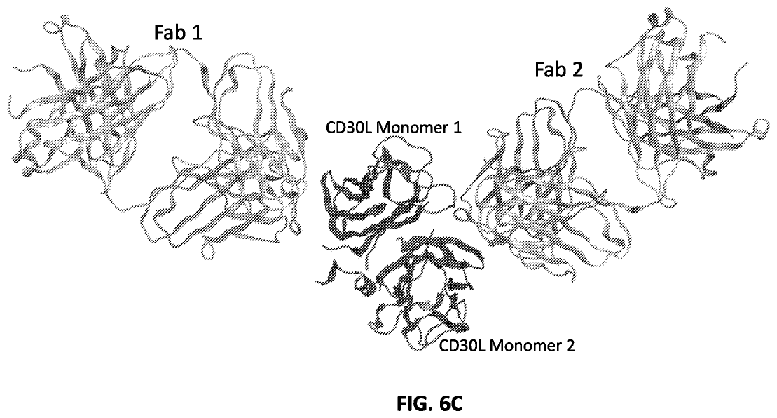

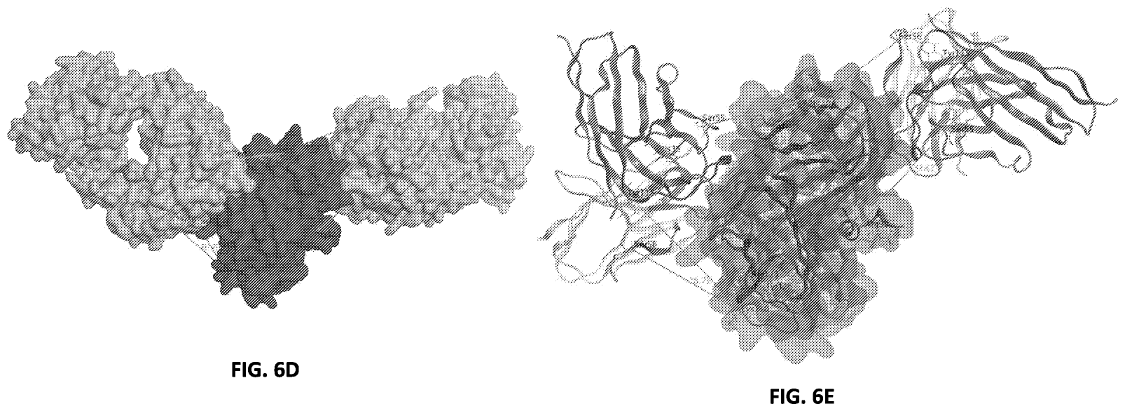

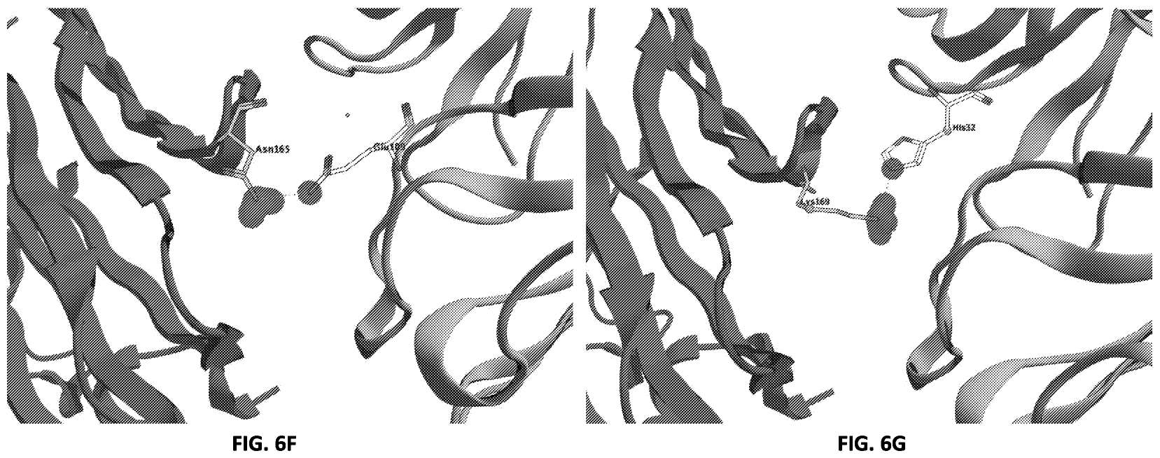

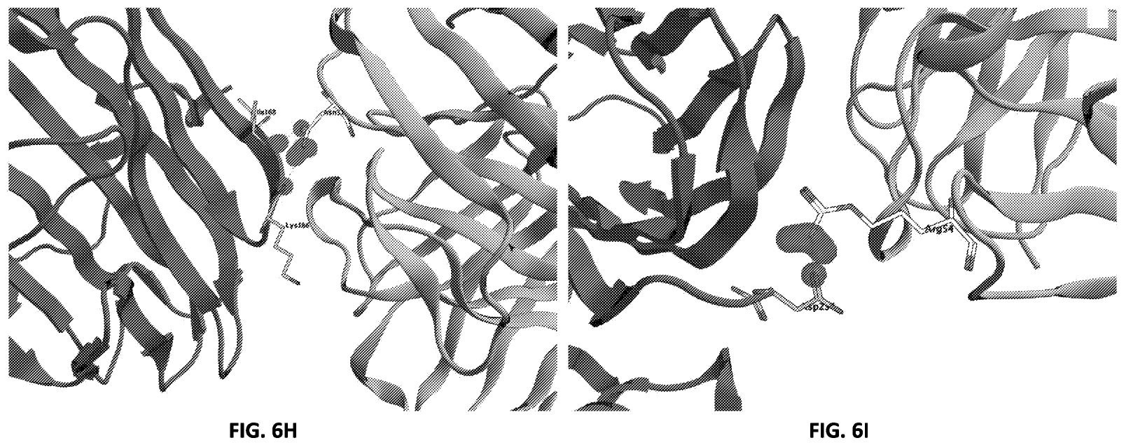

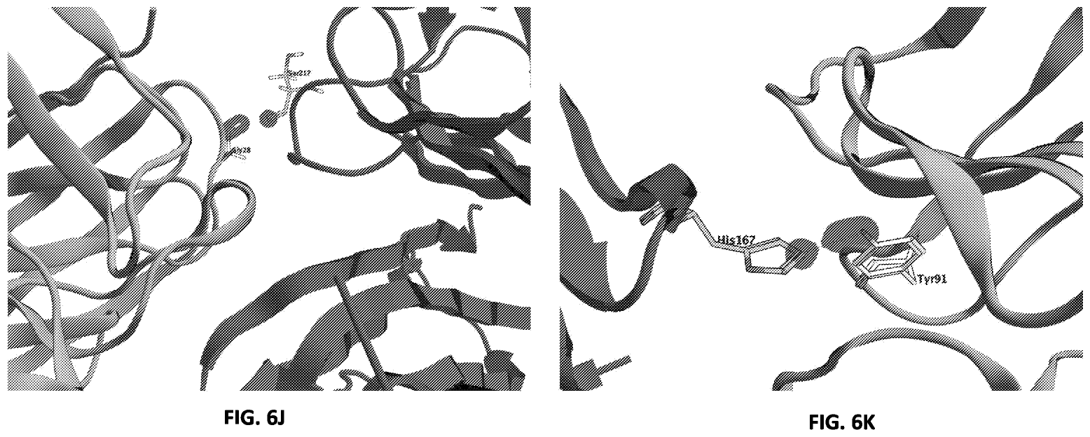

The high resolution CryoEM structure supports that the epitope and paratope residues primarily responsible for contact at the antibody:antigen interface comprise the amino acids denoted in Table 77 and displayed in FIGS. 6A and 6B. The structure also suggests additional contact points between the amino acids denoted in Table 78. The distance, energy, and the buried surface area as calculated from the structure demonstrated that the residues listed in Table 77 are the CD30L epitope and germlined-clone-2 antibody paratope. The structure showed that two germlined-clone-2 antibody Fabs bind to a CD30L dimer in the complex, in which each Fab binds to each monomer of the CD30L, as shown in FIG. 6C. Accordingly, the interactions between CD30L and germlined-clone-2 antibody Fab were observed multiple times, e.g. Fab 1 with CD30L monomer 1 and Fab 2 with CD30L monomer 2, as shown in FIG. 6C. The primary contacts between the Fab 1 and the CD30L monomer 1 are consistent with the primary contacts between Fab 2 and the CD30L monomer 2, thereby confirming such primary contact amino acids (listed in Table 77) as the epitope of CD30L for the germlined-clone-2 antibody. Additionally, Fab 2 makes other contacts with CD30L at the residues listed in Table 78.

TABLE 77

| Germlined clone | CD30L (SEQ | Type of | Distance | Energy |
|---|---|---|---|---|
| 2 residue | ID NO: 33) | Interaction | (Å) | (kcal/mol) |
| HC R54 (CDRH2) | D234 | Ionic | 3.08 | −14.02 |
| LC H32 (CDRL1) | K169 | Hydrogen | 3.07 | −10.3 |
| HC E109 (CDRH3) | N165 | Hydrogen | 2.84 | −5.0 |
| LC N31 (CDRL1) | I168 | Hydrogen | 2.87 | −4.4 |
| LC N31 (CDRL1) | K166 | Hydrogen | 2.81 | −3.8 |

TABLE 78

| Germlined clone | CD30L (SEQ | Type of | Distance | Energy |
|---|---|---|---|---|
| 2 residue | ID NO: 33) | Interaction | (Å) | (kcal/mol) |
| LC Y91 (CDRL3) | H167 | Hydrogen | 2.84, 2.60 | −3.1, −2.9 |
| LC G28 (CDRL1) | S217 | Hydrogen | 2.84 | −4.7 |
| HC W106 (CDRH3) | D118 | Hydrogen | 3.55 | −0.90 |

Therefore, based on the CD30L/anti-CD30L antibody complex structure, the epitope for the representative anti-CD30L antibody (germlined clone 2) can be any or any combination of D234, K169, N165, I168, and K166 as set forth in SEQ ID NO: 33. Alternatively, the epitope for the representative anti-CD30L antibody (germlined clone 2) can be any or any combination of D234, K169, N165, I168, K166, H167, S217, and D118, as set forth in SEQ ID NO: 33. Furthermore, the epitope for the representative anti-CD30L antibody (germlined clone 2) can be any or any combination of H167, S217, and D118, as set forth in SEQ ID NO: 33.

To confirm the epitope, crosslinking studies were performed as described in Section 5.7, Example 7. Briefly, the high affinity protein-protein complex was formed, then incubated with deuterated (e.g. suberoyl d0d12) bifunctional cross-linkers. The cross-linker mixture may contain cross-linkers with 1-hydroxy-7-azabenzotriazole groups as reactive groups bracketing spacer arms of various length, e.g. 3 to 30 Å. The cross-linked complex was subjected to multienzymatic cleavage, such as Trypsin, Chymotrypsin, ASP-N, Elastase and Thermolysin proteolysis. After enrichment of the cross-linked peptides, the samples were analyzed by high resolution mass spectrometry (nLC-orbitrap MS/MS) and the data generated were analyzed using Xquest version 2.0 and Stavrox 3.6. software.

The results of such crosslinking studies have been described in details in Section 5.7, Example 7.

Mapping the crosslinked residues from the crosslinking study confirms the epitope/paratope determined from the above described CryoEM structure. Briefly, the crosslinking residues and the distance between the crosslinked residues as determined from the cryoEM structure are shown in Table 79. The total internuclear distance between cross-links include the sidechains, product of the reactive groups, and the spacer arm. As an approximation for such cross-links, Ca—Ca distances were measured in the Cryo-EM structure and depicted as straight lines connecting the labeled residues in FIG. 6D and FIG. 6E. Some residues of the CD30L protein in the complex are disordered in the Cryo-EM experiment and therefore not modeled; where this applies no distance is reported. The cross-links identified by XL-MS between CD30L and germlined clone 2 Fab are within the expected distance range around the periphery of the non-covalent interactions in antibody-antigen interface found in the Cryo-EM structure. See FIG. 6D and FIG. 6E. As such, the results from the crosslinking studies provide a complementary description of the epitope-paratope interaction.

TABLE 79

| | CD30L SEQ ID 34 | |
|---|---|---|
| | (numbering based | |
| Germlined clone | on SEQ ID NO: 33 | Cα-Cα |
| 2 residue | in the bracket) | Distance (Å) |
| HC H35 (CDRH1) | R30 (R92) | 33 |
| HC S49 (FR-CDRH2) | S21 (S83) | NA |
| HC S55 (CDRH2) | R63, Y64 (R125, Y126) | 21-22 |
| HC Y108 (CDRH3) | S21 (S83) | NA |
| HC Y113 (CDRH3) | K48 (K110) | 33 |
| LC S52 (CDRL2) | S21 (S83) | NA |
| LC S56 (CDRL2) | K48 (K110) | 26-27 |
| LC T93 (CDRL3) | K16 (K78) | NA |

Close-up views of the contact sites were shown in FIGS. 6F-6K.

Based on the epitope mapped from cryoEM structure, the disclosure further provides that the germlined clone 2 binds to an epitope that is present in CD30L isoform 1 but missing in CD30L isoform 2. Accordingly, the disclosure confirmed that some embodiment of the antibody provided herein binds to CD30L isoform 1 but not isoform 2.

### 5.13 Example 13. Competition of Binding to CD30L Among Representative Functional Blocking Anti-CD30L Antibodies

As further described above, 110 clones across multiple antibody campaigns with at least three different mouse strains were generated, with the vast majority of which demonstrating robust binding to recombinant CD30L and cell surface CD30L and blocking/inhibiting recombinant CD30-CD30L binding. Despite such large number of clones from diverse campaigns and background, only several anti-CD30L both blocked/inhibited CD30-CD30L recombination protein interaction and functionally blocked/inhibited CD30L-mediated CD30 signaling in a dual cell assay. The common structural (including the primary structure, e.g. amino acid sequence) features of these clones that confer them both the binding and functional blocking capability have been determined as described above in Section 5.6 (Example 5). The disclosure provides that such unique function-blocking properties of these anti-CD30L antibodies may be determined by the related or overlapping epitope on CD30L targeted by the common antibody structural features (e.g. common structural features described above in Section 5.6 (Example 5). Competition of binding to CD30L amongst certain function-blocking antibodies were performed to identify other representative anti-CD30L antibodies that bind to epitope overlapping with or related to the epitope described in Section 5.12 (Example 12).

Binding competition studies were conducted using CARTERRA SPR platform. Briefly, a purified anti-CD30L antibody clone was normalized in duplicate to 10 μg/mL, 5 μg/mL and 1 μg/mL in 10 mM NaOAc, pH 4.25 and covalently coupled to the HC30M chip. A solution containing 80 nM rhCD30L was then injected over the covalently coupled HC30M chip for binding. A second injection of individual dilutions of each antibody (any self or other anti-CD30L antibody to be evaluated for ability to sandwich (e.g. noncompete) or compete with the anti-CD30L on the chip for CD30L binding) at 10 μg/mL was injected over the chip. Data from the 10 μg/mL coupled surface was analyzed for antibody binding. Asymmetries in the binding may be observed in CARTERRA assays. Such asymmetries represent clones that behave differently in each orientation when used as an analyte (in solution) or a ligand (immobilized on the surface), which may be attributed to antibody-induced conformational changes in CD30L.

Table 80 shows the competition among anti-CD30L clones 1, 2, 63, 80 and 82. These function-blocking antibodies compete against each other in at least one assay configuration. Such competition of binding to CD30L among clones 1, 2, 63, 80 and 82 indicate that they bind to overlapping or related epitopes. Furthermore, such competition amongst clones 1, 2, 63, 80 and 82 confirms that anti-CD30L antibodies clones 1, 63, 80 and 82 bind to epitope overlapping with or related to the epitope of clone 2 described in Section 5.12 (Example 12).

TABLE 80
Results of epitope binning based on antibody competition for CD30L binding

| | | Analyte mAbs (Injected over chip surface following capture of | | | | |
|---|---|---|---|---|---|---|
| | | rhCD30L by Ligand mAb) | | | | |
| Anti-CD30L Clone IDs | | Clone 82 | Clone 1 | Clone 63 | Clone 2 | Clone 80 |
| Antibodies on | Clone 82 | Self-self | Competing | Competing | Competing | Competing |
| the chip | | interaction | | | | |
| | Clone 1 | Competing | Self-self | Competing | Competing | Competing |
| | | | interaction | | | |
| | Clone 63 | Competing | Competing | Self-self | Competing | Competing |
| | | | | interaction | | |
| | Clone 2 | noncompete | noncompete | Competing | Self-self | Competing |
| | | | | | interaction | |
| | Clone 80 | noncompete | noncompete | Competing | noncompete | Self-self |
| | | | | | | interaction |

### 5.14 Example 14: Generation of Surrogate Anti-Mouse CD30L Antibody

The function-blocking anti-human CD30L antibodies described above have poor cross-reactivity with mouse CD30L. Therefore, an independent antibody campaign in rat with mouse CD30L antigen was conducted to generate function-blocking anti-mouse CD30L antibodies for animal model studies. Screening identified clone 111 a function blocking rat anti-mouse CD30L antibody. Clone 111 has a VH sequence set forth in SEQ ID NO: 770 and a VL sequence set forth in SEQ ID NO: 771. Clone 111 can have a CDR-H1 sequence set forth in any one of SEQ ID NOS: 774-779 based on Chothia, AbM, Kabat, Contact, IMGT, or Aho numbering scheme, a CDR-H2 sequence set forth in any one of SEQ ID NOS: 780-785 based on Chothia, AbM, Kabat, Contact, IMGT, or Aho numbering scheme, a CDR-H3 sequence set forth in any one of SEQ ID NOS: 786-791 based on Chothia, AbM, Kabat, Contact, IMGT, or Aho numbering scheme, a CDR-L1 sequence set forth in any one of SEQ ID NOS: 792-797 based on Chothia, AbM, Kabat, Contact, IMGT, or Aho numbering scheme, a CDR-L2 sequence set forth in any one of SEQ ID NOS: 798-803 based on Chothia, AbM, Kabat, Contact, IMGT, or Aho numbering scheme, and/or a CDR-L3 sequence set forth in any one of SEQ ID NOS: 804-809 based on Chothia, AbM, Kabat, Contact, IMGT, or Aho numbering scheme. To avoid immunogenicity in mouse studies, a VH/VL domain of the rat anti-mouse-CD30L antibody were fused with mouse IgG2a constant regions and produced as a rat-mouse chimeric antibody. Such full-length clone 111 (rat-mouse chimeric) anti-mouse-CD30L antibody has a heavy chain sequence set forth in SEQ ID NO: 772 and a light chain sequence set forth in SEQ ID NO: 773. The sequences were set forth in Table 27.

Although the function blocking rat anti-mouse CD30L antibody was generated and identified via another independent antibody campaign and was screened specifically for the mouse reactivity that the other function-blocking anti-CD30L antibodies lacked, the CDRs of the rat anti-mouse CD30L antibody have similarities to the CDRs of the function-blocking antibodies described in the examples above. For example, the alignment of the CDRs (using Kabat numbering scheme as an example) of clone 111 and clone 2 germlined are show in Table 81 below and indicate (identities and similarities of CDRs up to 100%, and similarities no less than 65%).

TABLE 81
Exemplary alignment of CDRs (Kabat numbering) between Clone 111 and Clone 2

| | CDR-L1 | | CDR-L2 | | CDR-L3 | |
|---|---|---|---|---|---|---|
| | (Kabat Numbering) | | (Kabat Numbering) | | (Kabat Numbering) | |
| Alignment | CLONE_2 | RASQGISNHLA | CLONE_2 | AASSLQS | CLONE_2 | QQYVTYPLT |
| | | \|\|\|.\|.\|:\|: | | .\|::\|:. | | :\|\| \|\|\|\| |
| | CLONE_111 | LASQDIGNYLS | CLONE_111 | GATNLED | CLONE_111 | HQYYEYPLT |
| Residues | 91% | | 100% | | 78% | |
| with | | | | | | |
| similarity | | | | | | |
| (%) | | | | | | |
| (including | | | | | | |
| ″\|″, ″:″, | | | | | | |
| and ″.″) | | | | | | |
| Residues | 73% | | 71% | | 78% | |
| with high | | | | | | |
| similarity | | | | | | |
| (%) | | | | | | |
| (including | | | | | | |
| ″\|″ and ″:″) | | | | | | |
| Identical | 55% | | 29% | | 67% | |
| Residues | | | | | | |
| (%) | | | | | | |
| (including | | | | | | |
| ″\|″ | | | | | | |
| | CDR-H1 | | CDR-H2 | | CDR-H3 | |
| | (Kabat Numbering) | | (Kabat Numbering) | | (Kabat Numbering) | |
| Alignment | CLONE_2 | DYAMH | CLONE_2 | GISWRSGNIGYADSVKG | CLONE_2 | DKGIGFNWNYEGFDY |
| | | \|\|\|\|\| | | | | :\|..: .\|\|\|\|\| |
| | CLONE_111 | DYAMH | CLONE_111 | SIIPLIDTTSYNQKFKG | CLONE_111 | ---LGSRY-LEGFDY |

| | | | |
|---|---|---|---|
| Residues | 100% | 65% | 100% |
| with some | | | |
| similarity | | | |
| (%) | | | |
| (including | | | |
| ″\|″,″:″, | | | |
| and ″.″) | | | |
| Residues | 100% | 29% | 73% |
| with high | | | |
| similarity | | | |
| (%) | | | |
| (including | | | |
| ″\|″ and ″:″) | | | |
| Identical | 100% | 24% | 55% |
| Residues | | | |
| (%) | | | |
| (including | | | |
| ″\|″) | | | |

″|″ indicates identity; ″:″ and ″.″ indicates different degrees of similarity, with ″:″ indicating higher degree of similarity.

### 5.15 Example 15: Studies of CD30L Blockade in CD4+ T Cell-Induced Murine Colitis and Inflammatory Cytokine Production In Vivo

The studies in this Example determines whether therapeutic CD30L blockade with the anti-CD30L antibodies provided herein have a protective effect in CD4+ T cell-induced model of murine colitis in vivo.

Briefly, the CD4+ T cell-induced model of murine colitis was used to assess the protective effect of CD30L blockade using mouse-specific CD30L blocking antibodies, clone_111 (HC sequence as set forth in SEQ ID NO: 772 and LC sequence as set forth in SEQ ID NO: 773). To initiate colitis, CD4+CD45RBhi T cells from the spleens of C57BL/6 mice were purified by magnetic activated cell sorting and fluorescence activated cell sorting to >95% purity and transferred by intraperitoneal (I.P.) injection to Rag2−/− mice lacking mature B and T cells (0.4×10⁶ cells/mouse). On the day of T cell injection, study day 0, recipient Rag2−/− mice were randomized into 6 treatment groups by weight. Mice were weighed and monitored for signs of disease and morbidity twice per week for 49 days.

Starting on study day 0, mice in the 6 treatment groups were treated by I.P. injection according to Table 82. Group 1 served as the naïve control and group 2 and 3 served as the vehicle and isotype antibody (same isotype as Clone_111) treated controls, respectively. Group 4 tested prophylactic treatment with a clinically relevant positive control for prevention of T cell-induced murine colitis, anti-IL-12p40. Group 5 tested therapeutic treatment, starting on study day 21, with Clone_111, a mouse specific CD30L blocking antibody on an Fc-silent backbone (HC sequence as set forth in SEQ ID NO: 772 and LC sequence as set forth in SEQ ID NO: 773). Group 6 tested prophylactic treatment with the same Clone_111.

TABLE 82

| | | | | Dose | | | | Dose | Dose |
|---|---|---|---|---|---|---|---|---|---|
| | # | Colitis | | level | Dose | | Dosing | volume | concentration |
| Group | mice | induction? | Treatment | (mg/kg)* | route | Regimen | days | (ml/kg)* | (mg/ml) |
| 1 | 5 | N | Naïve | N/A | N/A | N/A | N/A | N/A | N/A |
| 2 | 10 | Y | Vehicle | N/A | I.P. | 1x | D 0- | 10 | N/A |
| | | | (PBS) | | | weekly | D 49 | | |
| 3 | 10 | Y | Isotype | 10 | I.P. | 1x | D 0- | 10 | 1 |
| | | | control | | | weekly | D 49 | | |
| 4 | 10 | Y | Anti-IL- | 0.5 mg/mouse | I.P. | 1x | D 0- | 10 | 2.5 |
| | | | 12p40 | | | weekly | D 49 | | |
| 5 | 10 | Y | Clone 111 | 10 | I.P. | 1x | D 21- | 10 | 1 |
| | | | (anti- | | | weekly | D 49 | | |
| | | | CD30L) | | | | | | |
| 6 | 10 | Y | Clone_111 | 10 | I.P. | 1x | D 0- | 10 | 1 |
| | | | (anti- | | | weekly | D 49 | | |
| | | | CD30L) | | | | | | |

*Doses calculated daily in mg/kg based on the latest body weight measurement of each animal

At day 48 after disease induction, mice were assessed for intestinal pathology by endoscopic examination including photographs and scoring based on the criteria in Table 83. At day 49 after disease induction, mice were euthanized by bleeding to exsanguination followed by cervical dislocation. Half of the blood from each mouse was collected in Eppendorf tubes kept on ice for 30 minutes then spun down to collect the serum, which was stored on dry ice or at −80° C. until assessed by MSD for cytokine levels. The other half of the blood was collected into K2EDTA anticoagulant to collect plasma, which was stored on dry ice or at −80° C. Colons were harvested and measured for length and weight. A 1 cm piece of the middle colon was removed and stored in 1 ml of RNAlater on dry ice or at −80° C. until processing for gene expression studies. The remaining proximal and distal colon were preserved in 10% NBF for 24 hours, then transferred to 70% ethanol prior to processing for H&E staining and histopathological examination.

TABLE 83

| Endoscopic scoring | |
|---|---|
| system | |
| 0 | Normal |
| 1 | Moderate Loss of Vascularity |
| | (Small Vessels cannot be seen) |
| 2 | Severe Loss of Vascularity (Large & |
| | Small Vessels cannot be seen), |
| | Presence of Contact Bleeding |
| 3 | Presence of Mucosal Erosions |
| 4 | Ulcerations and/or Gross Bleeding |

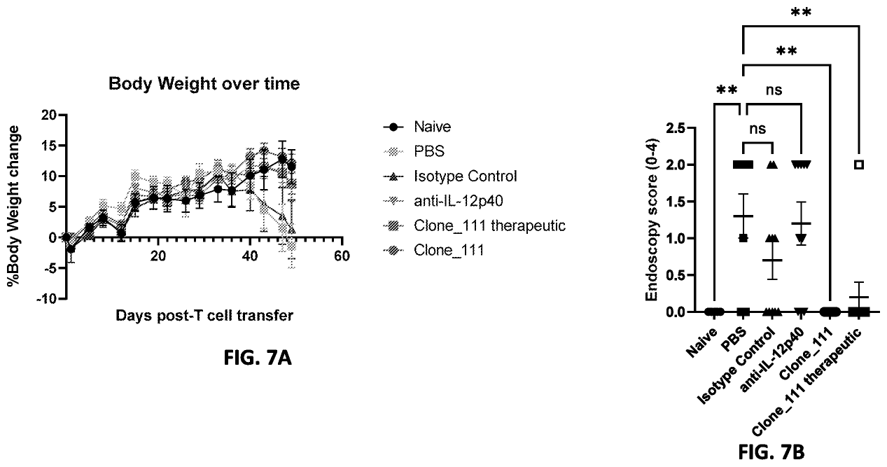

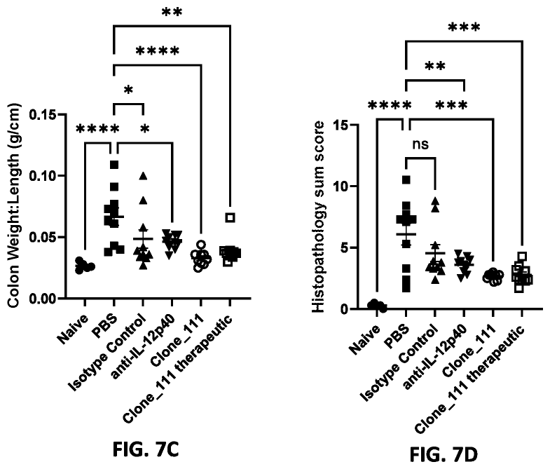

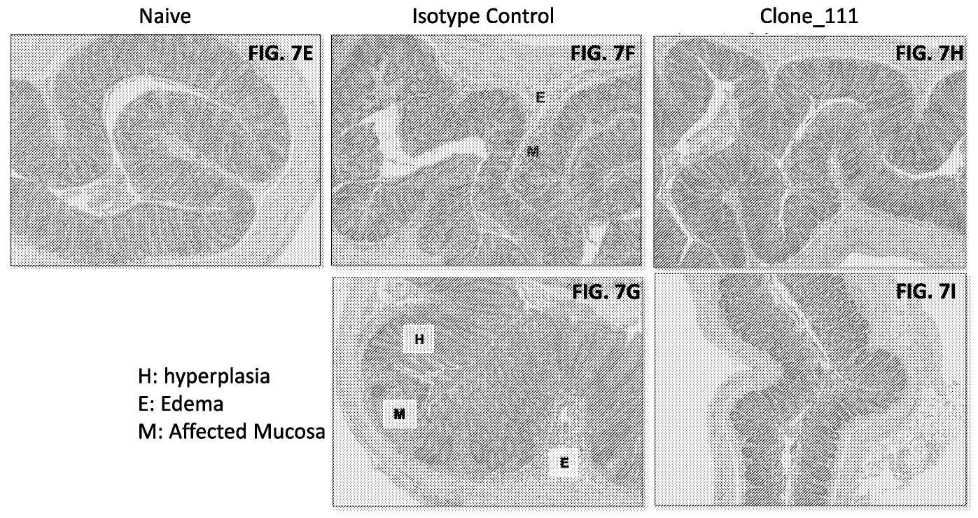

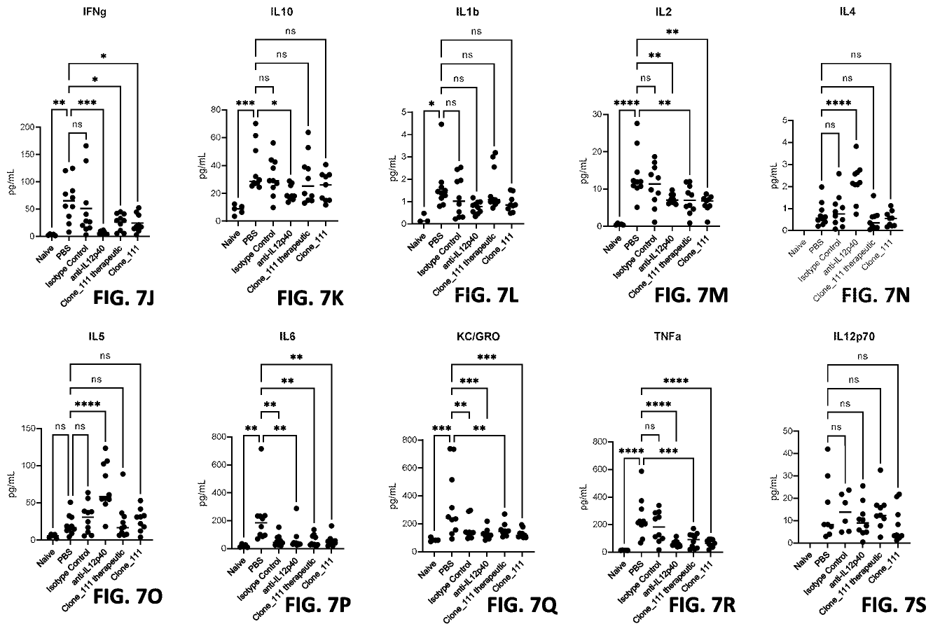

Body weight loss was observed starting at study day 40 in the PBS and isotype control antibody treated mice compared with naïve animals (FIG. 7A, grey, blue, and black lines, respectively). No weight loss was observed in the anti-IL-12p40 treated animals (FIG. 7A, green line). Prophylactic CD30L blockade by Clone_111 were effective in preventing weight loss, as was therapeutic CD30L blockade by Clone_111 (FIG. 7A, orange, red, purple lines, respectively).

Endoscopic assessment of gross intestinal pathology was used to assess severity of colitis at a macroscopic level. Moderate to severe loss of vascularity (endoscopy scores of 1-2) was observed in PBS and isotype control antibody treated animals compared with naïve mice (FIG. 7B). In contrast to its prevention of weight loss, anti-IL-12p40 treatment was not effective at significantly reducing macroscopic colon pathology. Surprisingly, prophylactic CD30L blockade by Clone_111 and therapeutic CD30L blockade by Clone_111 performed better than the positive control of anti-IL-12p40 treatment and were effective at reducing macroscopic colon pathology, with the majority of animals exhibiting normal colons at a macroscopic level (FIG. 7B).

As an additional measure of gross intestinal pathology, colon weight and length are measured and presented as a ratio of weight:length. This ratio increases with the inflammation associated with colitis. As expected, the colon weight:length ratio was significantly higher in PBS treated mice (reflecting induced colitis) than in naïve mice, while isotype antibody control treatment had no significant effect on the colon weight:length ratio as induced by colitis (FIG. 7C). Anti-IL-12p40 treatment positive control significantly reduced colon weight:length ratios, as did prophylactic and therapeutic CD30L blockade with Clone_111. Based on these data and the reduction in endoscopy scores, therapeutic or prophylactic CD30L blockade with the antibody provided herein can significantly reduce T cell-induced murine colitis.

To measure microscopic intestinal pathology, proximal and distal colon sections were stained with H&E and assessed by a veterinary pathologist for inflammation, gland loss, hyperplasia, and edema, the combined scores of which were represented as the histopathology sum score. PBS treated animals have significantly higher histopathology sum scores than naïve mice (FIG. 7D), reflecting the pathological damages of the colitis. The histopathology sum score is significantly reduced by IL-12p40 blockade, consistent with the published role for IL-12 and IL-23 in murine colitis. CD30L blockade by Clone_111 prevented microscopic intestinal pathology to a similar extent as the positive control of anti-IL-12p40, and this effect was observed not only in prophylactic CD30L blockade (anti-CD30L treatment before or at the onset of T cell transfer), but also for therapeutic CD30L blockade (anti-CD30L treatment after the induction of colitis), indicating an ongoing role for CD30-CD30L interactions during the development of colitis. Representative images from the proximal colon of naïve, isotype control, and Clone_111 treated mice were shown in FIGS. 7E-7I and confirmed that CD30L blockade reduced and reversed intestinal pathology in colitis.

A characteristic hallmark of T cell-induced colitis in mice is the elevation of systemic levels of proinflammatory cytokines. These levels are detectable in the serum of diseased mice, and the reduction of proinflammatory cytokines is an important hallmark of efficacious anti-colitogenic therapeutics. Significantly elevated levels of IFNg, IL-10, IL-1b, IL-2, IL-6, KC/Gro, TNFa, and IL-12p70 were observed in mice with T cell-induced colitis treated with PBS or isotype control antibodies (FIGS. 7J-7S). The positive control anti-IL-12p40 treatment significantly reduced all but IL-12p70, with a concomitant increase in the type 2 cytokines IL-4 and IL-5. Prophylactic or therapeutic CD30L blockade with Clone_111 significantly reduced IFNg, IL-2, IL-6, KC/Gro, and TNFa, without effecting levels of IL-4 or IL-5. These data suggest that the observed anti-colitogenic effect of CD30L blockade may be due to inhibition of pathogenic immune responses in a mechanism distinct from that of IL-12p40 blockade.

### 5.16 Example 16: Comparing the Binding of Anti-CD30L Antibody to CD30L Isoform 1 and CD30L Isoform 2

This study compares the binding of CD30L isoform 1 and CD30L isoform 2 with the anti-CD30L antibodies or antigen binding fragments provided herein. By such a comparison, this study validates that the disclosure provides an anti-CD30L antibody or antigen binding fragment that binds to CD30L isoform 1 but not CD30L isoform 2.

Briefly, CD30L isoforms 1 or 2 were separately expressed by transfecting HEK293 cells with CMV vectors encoding Flag-tagged CD30L isoforms 1 or 2 with PEI transfection agent. The binding of a representative anti-CD30L clone (germlined clone 2) to CD30L isoforms 1 or 2 expressed on HEK293 cells was assessed by flow cytometry using anti-CD30L (germlined clone 2) conjugated to AF647. Expression of Flag-tagged CD30L isoform 1 and isoform 2 at the surface of HEK cells was detected using FITC-conjugated anti-Flag-tag antibodies. Co-staining of the FITC and AF647 would indicate binding of the anti-CD30L to the expressed CD30L isoform. Staining of FITC but not AF647 would indicate that the cells had robust expression of the transfected CD30L isoform but had no binding by the anti-CD30L.

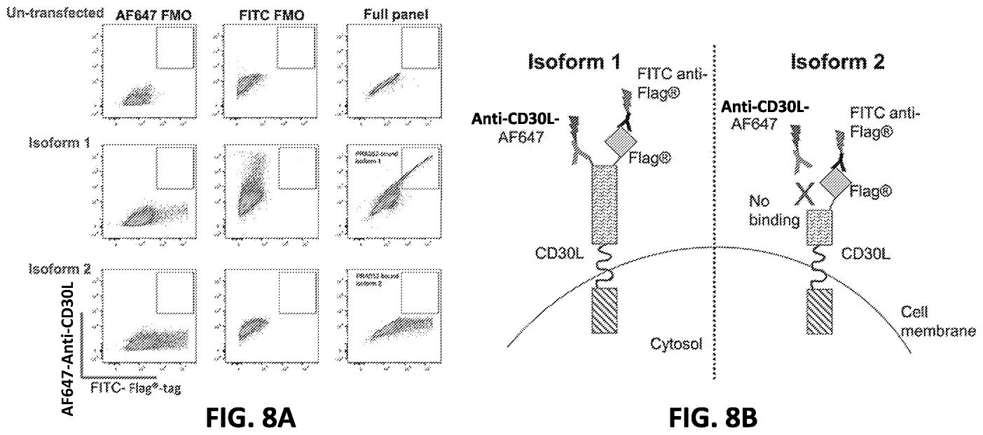

As shown in FIG. 8A, analyses of CD30L-isoform-1-expressing cells (CD30L isoform 1 FITC-Flag-tag positive cells) showed that 96.6% of the FITC positive cells were also positive for AF647 conjugated anti-CD30L (germlined clone 2), indicating strong binding of the anti-CD30L (germlined clone 2) to CD30L isoform 1. In contrast, only virtually none (0.04%) of the CD30L-isoform-2-expressing cells (CD30L isoform 2 FITC-Flag-tag positive cells) were positive for AF647 conjugated anti-CD30L (germlined clone 2), indicating lack of binding of the anti-CD30L (germlined clone 2) to CD30L isoform 2. These results validate that anti-CD30L clone (germlined clone 2) binds to CD30L isoform 1 but does not bind to CD30L isoform 2, as summarized in FIG. 8B.

### 5.17 Example 17: Long Term Stability Studies of Anti-CD30L Formulation

The disclosure further provides studies that confirm the long term stability of the formulation selected in Example 10 (Section 5.10) for the representative anti-CD30L clone (germlined clone 2). As is known and practiced in the art, the formulation with long term stability provides the advantage of longer shelf life and longer expiration date for clinical use. Additionally, the formulation can be stable in higher temperature (e.g. 4° C. as opposed to −70° C.) provides the advantage of easier storage and transportation.

Briefly, the selected formulation was tested in a 12-month (12M) stability study under various conditions (5° C.±3° C., or −70° C.±10° C.), as listed in Table 84 below.

TABLE 84
Formulations tested in 12-M stability study

| | | Formulation | Storage | |
|---|---|---|---|---|
| | Anti-CD30L | concentration | conditions | Formulation |
| Studies | antibodies | and format | tested | tested |
| Study #1 | anti-CD30L | 200.0 mg/mL; | 5° C. ± 3° C., | 20 mM Histidine |
| | germlined | 2.5 mL/vial | Upright vial, | buffer, 100 mM |
| | clone 2 | | protected from | L-Arginine |
| | | | light | Monohydrochloride, |
| | | | | 0.02% (w/v) PS20, |
| | | | | pH 5.5 |
| Study #2 | anti-CD30L | 200.0 mg/mL; | −70° C. ± 10° C., | 20 mM Histidine |
| | germlined | 16 mL/Bag | flatwise | buffer, 100 mM |
| | clone 2 | | | L-Arginine |
| | | | | Monohydrochloride, |
| | | | | 0.02% (w/v) PS20, |
| | | | | pH 5.5 |

Testing items including appearance (color and clarity, both using instrumental method), pH, protein conc., iCIEF, size exclusion chromatography (SEC), reduced CE-SDS, non-reduced CE-SDS, and binding ELISA (binding to CD30L) were conducted. Samples were obtained and analyzed at the beginning of the study (T0), 1 month of storage (1M), 3 months of storage (3M), 6 months of storage (6M), 9 months of storage (9M), and 12 months of storage (12M), with the result described below in Table 85 for the 5° C.±3° C. storage condition and in Table 86 for the −70° C.±10° C. storage condition.

TABLE 85
12 M stability results of Study #1 in Table 84 (5° C ± 3° C storage)

| | Sub-test | Acceptance | T0 (Time | | | | | |
|---|---|---|---|---|---|---|---|---|
| Test Items | items | Criteria | zero) | 1 M | 3 M | 6 M | 9 M | 12 M |
| Color | Color | Not more | Equal | Equal | Equal | Equal | Equal | Equal |
| | | colored than | colored | colored to | colored to | colored to | colored to | colored to |
| | | B1 | to M | M | M | M | M | M |
| Clarity | Clarity | ≤30. NTU | 13.5 | 13.6 | 13.9 | 13.8 | 14.0 | 14.1 |
| Visible | Visible | Liquid, | Liquid, | Liquid, | Liquid, | Liquid, | Liquid, | Liquid, |
| particle | particle | essentially | essentially | essentially | essentially | essentially | essentially | essentially |
| | | free of | free of | free of | free of | free of | free of | free of |
| | | visible | visible | visible | visible | visible | visible | visible |
| | | particles | particles | particles | particles | particles | particles | particles |
| pH | pH | 5.0-6.0 | 5.5 | 5.5 | 5.5 | 5.5 | 5.6 | 5.6 |
| Subvisible | ≥10 μm | ≤6000 particle(s)/ | 6 | 5 | 0 | 126 | 293 | 464 |
| Particulate | | container | | | | | | |
| Matter | ≥25 μm | ≤600 particle(s)/ | 0 | | 0 | 0 | 37 | 5 |
| | | container | | | | | | |
| | ≥2 μm | | 228 | 127 | 43 | 910 | 1960 | 3927 |
| | ≥5 μm | | 41 | 17 | 2 | 405 | 880 | 1500 |
| | ≥3 μm | | 154 | 70 | 23 | 719 | 1547 | 2942 |
| Protein | Protein | 180.0-220.0 | 197.0 | 197.0 | 195.7 | 198.5 | 197.3 | 198.4 |
| Concentration | Concentration | mg/mL | | | | | | |
| iCIEF | Main Peak | 8.3-9.3 | 8.8 | 8.8 | 8.9 | 8.8 | 8.9 | 8.8 |
| | pI | | | | | | | |
| | Main | ≥40.0% | 73.9 | 71.5 | 71.3 | 71.5 | 71.6 | 68.0 |
| | Peak % | | | | | | | |
| | Acidic | ≤60.0% | 19.9 | 22.5 | 22.0 | 21.9 | 22.2 | 25.3 |
| | peaks % | | | | | | | |
| | Basic | ≤20.0% | 6.2 | 6.1 | 6.8 | 6.6 | 6.2 | 6.7 |
| | peaks % | | | | | | | |
| SEC | Main Peak | ≥95.0% | 99.4 | 99.5 | 99.1 | 98.9 | 99.0 | 98.9 |
| | (monomer)% | | | | | | | |
| | HMWS % | ≤5.0% | 0.5 | 0.5 | 0.7 | 0.7 | 0.9 | 0.9 |
| Reduced | LC + HC | ≥90.0% | 98.4 | 97.9 | 98.1 | 98.1 | 98.2 | 98.0 |
| CE-SDS | Total | | 1.6 | 2.1 | 1.9 | 1.9 | 1.8 | 2.0 |
| | Minor | | | | | | | |
| | Species % | | | | | | | |
| | HC % | | 66.9 | 67.9 | 67.8 | 67.0 | 65.9 | 67.1 |
| | LC % | | 31.5 | 30.0 | 30.2 | 31.1 | 32.3 | 30.9 |
| Non- | Main Peak | ≥85.0% | 98.3 | 98.3 | 98.2 | 97.7 | 97.8 | 98.0 |
| reduced | Sum of | ≤15.0% | 1.7 | 1.7 | 1.7 | 2.3 | 2.2 | 1.8 |
| CE-SDS | LMW | | | | | | | |
| | Forms | | | | | | | |
| Binding | Binding | 60-140% | 101% | 96% | 103% | 104% | 97% | 103% |
| ELISA | ELISA | Relative | relative | relative | relative | relative | relative | relative |
| | | Potency | potency | potency | potency | potency | potency | potency |

TABLE 86
12 M stability results of Study #2 in Table 84 (−70° C. ± 10° C. storage)

| | Sub-test | Acceptance | T0 (Time | | | | | |
|---|---|---|---|---|---|---|---|---|
| Test Items | items | Criteria | zero) | 1 M | 3 M | 6 M | 9 M | 12 M |
| Color | Color | Not more | Equal | Equal | Equal | Equal | Equal | Equal |
| | | colored than | colored | colored to | colored to | colored to | colored to | colored to |
| | | B1 | to M | M | M | M | M | M |
| Clarity | Clarity | ≤30. NTU | 13.5 | 13.8 | 13.8 | 13.9 | 14.0 | 13.7 |
| Visible | Visible | Liquid, | Liquid, | Liquid, | Liquid, | Liquid, | Liquid, | Liquid, |
| particle | particle | essentially | essentially | essentially | essentially | essentially | essentially | essentially |
| | | free of | free of | free of | free of | free of | free of | free of |
| | | visible | visible | visible | visible | visible | visible | visible |
| | | particles | particles | particles | particles | particles | particles | particles |
| pH | pH | 5.0-6.0 | 5.5 | 5.5 | 5.6 | 5.5 | 5.5 | 5.5 |
| Protein | Protein | 180.0-220.0 | 196.0 | 197.8 | 197.2 | 199.6 | 196.3 | 198.9 |
| Concentration | Concentration | mg/mL | | | | | | |
| iCIEF | Main Peak | 8.3-9.3 | 8.8 | 8.8 | 8.8 | 8.8 | 8.8 | 8.9 |
| | pI | | | | | | | |
| | Main | ≥40.0% | 71.9 | 72.2 | 71.0 | 71.0 | 72.6 | 71.9 |
| | Peak % | | | | | | | |
| | Acidic | ≤60.0% | 21.5 | 21.2 | 21.3 | 22.8 | 21.6 | 21.6 |
| | peaks % | | | | | | | |
| | Basic | ≤20.0% | 6.6 | 6.5 | 7.7 | 6.2 | 5.9 | 6.5 |
| | peaks % | | | | | | | |
| SEC | Main Peak | ≥95.0% | 99.5 | 99.6 | 99.5 | 99.6 | 99.6 | 99.6 |
| | (monomer)% | | | | | | | |
| | HMWS % | ≤5.0% | 0.4 | 0.4 | 0.5 | 0.4 | 0.4 | 0.4 |
| Reduced | LC + HC | ≥90.0% | 98.3 | 97.9 | 98.1 | 97.9 | 97.6 | 98.0 |
| CE-SDS | Total | | 1.7 | 2.1 | 1.9 | 2.1 | 2.4 | 2.0 |
| | Minor | | | | | | | |
| | Species % | | | | | | | |
| | HC % | | 67.0 | 67.6 | 67.8 | 68.0 | 66.7 | 68.1 |
| | LC % | | 31.3 | 30.3 | 30.3 | 29.9 | 30.9 | 29.9 |
| Non- | Main Peak | ≥85.0% | 98.3 | 98.0 | 98.2 | 98.2 | 97.9 | 98.2 |
| reduced | | | | | | | | |
| CE-SDS | Sum of | ≤15.0% | 1.7 | 2.0 | 1.8 | 1.8 | 2.1 | 1.8 |
| | LMW | | | | | | | |
| | Forms | | | | | | | |
| Binding | Binding | 60-140% | 104% | 107% | 101% | 101% | 96% | 100% |
| ELISA | ELISA | Relative | relative | relative | relative | relative | relative | relative |
| | | Potency | potency | potency | potency | potency | potency | potency |

Accordingly, the anti-CD30L formulation described above have been demonstrated to be stable for 12 months when stored at 5° C.±3° or −70° C.±10°, with concentration, binding potency, pI, percentage of monomer, and other analytical characteristics stable for the duration of the 12-month storage.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### 6. SEQUENCE LISTING

TABLE 27
Sequence Listing

| SEQ | | Description | Description | Description |
|---|---|---|---|---|
| ID | SEQUENCE | 1 | 2 | 3 |
| 1 | QVQLQESGPGLVKPSETLSLTCTVS | CLONE_1_VH_ | | |
| | GGSISGYYWSWIRQPPVKGLEWIGY | parental | | |
| | IFYSGYTKYNPSLKSRVTISVDTSK | | | |
| | NQFSLKLSSVTAADTAVYYCARDRW | | | |
| | DFDYWGQGALVTVSS | | | |
| 2 | QVQLQESGPGLVKPSETLSLTCTVS | CLONE_1_VH_ | | |
| | GGSISGYYWSWIRQPPGKGLEWIGY | germlined | | |
| | IFYSGYTKYNPSLKSRVTISVDTSK | | | |
| | NQFSLKLSSVTAADTAVYYCARDRW | | | |
| | DFDYWGQGTLVTVSS | | | |
| 3 | AIQMTQSPSYLSASVGDRVTIACRA | CLONE_1_VL_ | | |
| | SQGIRNNLGWYQQKPGKAPKLLIYA | parental | | |
| | ESSLQSGVPSRFSGSGSGTDFTLTI | | | |
| | SSLQPEDFATYYCLQDFNYPYTFGQ | | | |
| | GTKLEIK | | | |
| 4 | AIQMTQSPSSLSASVGDRVTITCRA | CLONE_1_VL_ | | |
| | SQGIRNNLGWYQQKPGKAPKLLIYA | germlined | | |
| | ESSLQSGVPSRFSGSGSGTDFTLTI | | | |
| | SSLQPEDFATYYCLQDFNYPYTFGQ | | | |
| | GTKLEIK | | | |
| 5 | EVQLVESGGGLVQPGRSLRLSCAAS | CLONE_2_VH_ | | |
| | GFNFDDYAMHWVRQAPGKGLEWVSG | parental | | |
| | ISWRSGNIGYAGSVKGRFTISRDNA | | | |
| | KNSLYLQMNSLRPEDSALYYCAKDK | | | |
| | GIGFNWNYEGFDYWGQGTLVTVSS | | | |
| 6 | EVQLVESGGGLVQPGRSLRLSCAAS | CLONE_2_VH_ | | |
| | GFNFDDYAMHWVRQAPGKGLEWVSG | germlined | | |
| | ISWRSGNIGYADSVKGRFTISRDNA | | | |
| | KNSLYLQMNSLRAEDTALYYCAKDK | | | |
| | GIGFNWNYEGFDYWGQGTLVTVSS | | | |
| 7 | DIQMTQSPSSLSASVGDRVTITCRA | CLONE_2_VL_ | | |
| | SQGISNHLAWFQQKPGKAPKSLIYA | parental | | |
| | ASSLQSGVPSKFSGSGSGTDFTLTI | | | |
| | SSLQPEDFATYYCQQYVTYPLTFGG | | | |
| | GTKVEIK | | | |
| 8 | DIQMTQSPSSLSASVGDRVTITCRA | CLONE_2_VL_ | | |
| | SQGISNHLAWFQQKPGKAPKSLIYA | germlined | | |
| | ASSLQSGVPSRFSGSGSGTDFTLTI | | | |
| | SSLQPEDFATYYCQQYVTYPLTFGG | | | |
| | GTKVEIK | | | |
| 9 | QVQLQESGPGLVKPSETLSLTCTVS | CLONE_3_VH_ | | |
| | GGSISGYYWSWIRQPPVKGLEWIGY | parental | | |
| | IFYSGYTKYNPSLKSRVTISVDTSK | | | |
| | NQFSLKLSSVTAADTAVYYCARDRW | | | |
| | DFDYWGQGALVTVSS | | | |
| 10 | QVQLQESGPGLVKPSETLSLTCTVS | CLONE_3_VH_ | | |
| | GGSISGYYWSWIRQPPGKGLEWIGY | germlined | | |
| | IFYSGYTKYNPSLKSRVTISVDTSK | | | |
| | NQFSLKLSSVTAADTAVYYCARDRW | | | |
| | DEDYWGQGTLVTVSS | | | |
| 11 | DIVMTQSPLSLPVTPGEPASFSCRS | CLONE_3_VL_ | | |
| | SQSLLHSNGYNYLDWYLQKPGQSPQ | parental | | |
| | LLIYLGSNRASGVPDRFSGSGSGTD | | | |
| | FTLKISRVEAADVGVYYCMQALQTP | | | |
| | RTFGQGTKVEIK | | | |
| 12 | DIVMTQSPLSLPVTPGEPASISCRS | CLONE_3_VL_ | | |
| | SQSLLHSNGYNYLDWYLQKPGQSPQ | germlined | | |
| | LLIYLGSNRASGVPDRFSGSGSGTD | | | |
| | FTLKISRVEAEDVGVYYCMQALQTP | | | |
| | RTFGQGTKVEIK | | | |
| 13 | QVQLVESGGGVVQPGRSLRLSCAAS | CLONE_4_VH_ | | |
| | GFPFSSYGMHWVRQAPGKGLEWVTF | parental | | |
| | IWEDGNNKDYADSVKGRFSVSRDNS | | | |
| | KNTLYLQMNSLRADDTAVYYCARNG | | | |
| | VYYGSGAYVDYWGQGTLVTVSS | | | |
| 14 | QVQLVESGGGVVQPGRSLRLSCAAS | CLONE_4_VH_ | | |
| | GFPFSSYGMHWVRQAPGKGLEWVTF | germlined | | |
| | IWFDGNNKDYADSVKGRFTISRDNS | | | |
| | KNTLYLQMNSLRAEDTAVYYCARNG | | | |
| | VYYGSGAYVDYWGQGTLVTVSS | | | |
| 15 | DIVMTQSPLSLPVTPGEPASFSCRS | CLONE_4_VL_ | | |
| | SQSLLHSNGYNYLDWYLQKPGQSPQ | parental | | |
| | LLIYLGSNRASGVPDRFSGSGSGTD | | | |
| | FTLKISRVEAADVGVYYCMQALQTP | | | |
| | RTFGQGTKVEIK | | | |
| 16 | DIVMTQSPLSLPVTPGEPASISCRS | CLONE_4_VL_ | | |
| | SQSLLHSNGYNYLDWYLQKPGQSPQ | germlined | | |
| | LLIYLGSNRASGVPDRFSGSGSGTD | | | |
| | FTLKISRVEAEDVGVYYCMQALQTP | | | |
| | RTFGQGTKVEIK | | | |
| 17 | QVQLVESGGGVVQPGRSLRLSCAAS | CLONE_59_ | | |
| | GFTFSIYGMHWVRQAPGKGLEWVAV | VH | | |
| | IWYDGSNKYYVDSVKGRFTISRDNS | | | |
| | KNSLYLQMNSLRAEDTAVYYCAREG | | | |
| | NLFDYWGQGTLVTVSS | | | |
| 18 | DIQMTQSPSTLSASVGDRVTITCRA | CLONE_59_ | | |
| | SQSISSWLAWYQQKPGKAPKLLIYK | VL | | |
| | ASSLESGVPSRFSGSGSGTEFTLTI | | | |
| | SSLKPDDFATYYCQQYKSYYTFGQG | | | |
| | TKLEIK | | | |
| 19 | QVHLVESGGGVVQPGRSLRLSCAAS | CLONE_60_ | | |
| | GFAFNIYGMHWVRQAPGKGLEWVAV | VH | | |
| | ISYDGSNKVYADSVKGRFSISRDNS | | | |
| | KNTLYLQMNSLRAEDTAVYYCAKSG | | | |
| | GITMVRGVFDYWGQGTLVTVSS | | | |
| 20 | DIQMTQSPSSLPASVGDRVTITCRA | CLONE_60_ | | |
| | SQGIRNDLGWYQQKPGKAPKRLIHA | VL | | |
| | ASSLHSGVPSRFSGSGSGTEFTLTI | | | |
| | SSLQPEDFATYYCLQHNRYPITFGQ | | | |
| | GTRLEIK | | | |
| 21 | QITLKESGPTLVKPTQTLTLTCTES | CLONE_61_ | | |
| | GFSLSTSGVGVGWIRQPPGKALEWL | VH | | |
| | ALIYWNDDKRYSPSLKSRLTITKDT | | | |
| | SKNQVVLTMTNMDPVDTATYYCAHR | | | |
| | RTTTVTIYYYYMDVWGKGTTVTVSS | | | |
| 22 | EIVMTQSPATLSVSPGERATLSCRA | CLONE_61_ | | |
| | SQSVSSNLAWYQQKPGQAPRLLIYG | VL | | |
| | ASTRATGIPARFSGSGSGTEFTLTI | | | |
| | SSLQSEDFAVYYCQQYNNWPFTFGP | | | |
| | GTKVDIK | | | |
| 23 | EVQLVESGGGLVQPGGSLRLSCAAS | CLONE_63_ | | |
| | GFMFSSYSMNWVRQAPGKGLEWVSY | VH | | |
| | ISSSSSTIYDADSVKGRFTISRDDA | | | |
| | KNSLYLQMNSLRDEDTAVYYCTREA | | | |
| | YPGYYYNYMDVWGKGTTVTVSS | | | |
| 24 | DIQMTQSPSSLSASVGDRVTITCRA | CLONE_63_ | | |
| | SQSISSRLAWYQQKPGKAPNLLIYK | VL | | |
| | ASNLESGVPSRFSGSGSGTEFTLTI | | | |
| | SSLQPDDFATYYCQQYNSYSRTFGQ | | | |
| | GTKVEIK | | | |
| 25 | EVQLVESGGGLVQPGGSLRLSCAAS | CLONE_64_ | | |
| | GFTFSNYAMTWVRQAPGKGLEWVSA | VH | | |
| | ISGFGGSTYYADSVKGRFTISRDNS | | | |
| | KNTLYLQMNSLRAEDTAVYYCAKDH | | | |
| | DYYAFDYWGQGTLVTVSS | | | |
| 26 | DIQMTQSPSSLSASVGDRVTITCRA | CLONE_64_ | | |
| | SQSISSYLNWYQQKPGKAPKLLIYA | VL | | |
| | VSSLQSGVPSRFSGSGSGTDFTLTI | | | |
| | SSLQPEDFATYYCQQSYSTPYTFGQ | | | |
| | GTKLEIK | | | |
| 27 | EVQLVESGGGLVQPGGSLKLSCAAS | CLONE_65_ | | |
| | GFTFSNYAMTWVRQAPGKGLEWVSA | VH | | |
| | ISGYGGSTYYAASVKGRFTISRDNS | | | |
| | KNTLFLQMNSLRAEDTAVYYCAKDH | | | |
| | DYYAFDYWGQGTLVPVSS | | | |
| 28 | DIQMTQSPSSLSASVGDRVTITCRA | CLONE_65_ | | |
| | SQSINSYLNWYQQKPGKAPKLLIYS | VL | | |
| | ASSLQSGVPSRFSGSGSGTDFTLTI | | | |
| | SSLQPEDFATYYCQQSYSTPYTFGQ | | | |
| | GTKLEIK | | | |
| 29 | EVQLVESGGGLVQPGGSLRLSCEVS | CLONE_80_ | | |
| | GFTFSSYDMHWVRQVTGTGLEWVSS | VH | | |
| | IGIGGDTYYPGSVKGRFTISRENAK | | | |
| | NSLYLQMNSLRGGDTGVYYCARGEW | | | |
| | DLLWYFMDVWGKGTTVTVSS | | | |
| 30 | DIQMTQSPSSLSASIGDRVTITCRA | CLONE_80_ | | |
| | SQGISSWLAWYQQKPGKAPKLLIYA | VL | | |
| | ASSLQSGVPSRFSGSGSGTDFTLTI | | | |
| | SSLQPEDFATYYCQQANSFPLTFGG | | | |
| | GTKVEIK | | | |
| 31 | EVQLVESGGGLVQPGGSLRLSCAVS | CLONE_82_ | | |
| | GITFSNAWMSWVRQAPGKGLEWVGR | VH | | |
| | IKSKTYGGTTDYAAPVKGRFIISRD | | | |
| | DSKDTLYLQMNSLKTEDTAIYYCTT | | | |
| | DPWNYVNYNYFMDVWGKGTTVTVSS | | | |
| 32 | DIQMTQSPSSLSASVGDRVTITCRA | CLONE_82_ | | |
| | SQDIRNYLAWYQQKPGKITHLLVYA | VL | | |
| | ASTLQSGVPSRFSGSGSGTDFTLTI | | | |
| | NSLQPEDVATYYCQNYFSVPLTFGG | | | |
| | GTKVEIK | | | |
| 33 | MDPGLQQALNGMAPPGDTAMHVPAG | CD30L full | | |
| | SVASHLGTTSRSYFYLTTATLALCL | length | | |
| | VFTVATIMVLVVQRTDSIPNSPDNV | sequence | | |
| | PLKGGNCSEDLLCILKRAPFKKSWA | | | |
| | YLQVAKHLNKTKLSWNKDGILHGVR | | | |
| | YQDGNLVIQFPGLYFIICQLQFLVQ | | | |
| | CPNNSVDLKLELLINKHIKKQALVT | | | |
| | VCESGMQTKHVYQNLSQFLLDYLQV | | | |
| | NTTISVNVDTFQYIDTSTEPLENVL | | | |
| | SIFLYSNSD | | | |
| 34 | QRTDSIPNSPDNVPLKGGNCSEDLL | CD30L | | |
| | CILKRAPFKKSWAYLQVAKHLNKTK | extra- | | |
| | LSWNKDGILHGVRYQDGNLVIQFPG | cellular | | |
| | LYFIICQLQFLVQCPNNSVDLKLEL | domain | | |
| | LINKHIKKQALVTVCESGMQTKHVY | sequence | | |
| | QNLSQFLLDYLQVNTTISVNVDTFQ | (isoform 1 | | |
| | YIDTSTFPLENVLSIFLYSNSD | of CD30L) | | |
| 35 | QRTDSIPNSPDNVPLKGGNCSEDLLCIL | Extra- | | |
| | KRAPFKKSWAYLQVAKHLNKTKLSWNKD | cellular | | |
| | GILHGVRYQDGNLVIQFPDYCGMILHHS | domain | | |
| | HSTLDSGKGHCCLETLQP | sequence | | |
| | | of isoform | | |
| | | 2 of CD30L | | |
| 766 | QVQLQESGPGLVKPSETLSLTCTVS | Reference- | | |
| | GGSISSYIWSWIRQPAGKGLEWIGR | 1 (Ref1)_VH | | |
| | IYASGNTNYNPSLKSRVTISVDTSK | | | |
| | NQFSLKLSSMTAADTAVYYCARDYR | | | |
| | VAGTYYYYYGLDVWGQGTTVTVSS | | | |
| 767 | QSALTQPASVSGSPGQSITISCTGT | Reference- | | |
| | SSDVGVYDYVSWYQQHPGKAPKLMI | 1 (Ref1)_VL | | |
| | YEVSNRPSGVSNRFSGSKSGNTASL | | | |
| | TISGLQTEDEADYYCSSYTSRSTWV | | | |
| | FGGGTKLTVL | | | |
| 768 | QVQLQESGPGLVKPSETLSLTCTVS | Reference- | | |
| | GGSISSYIWSWIRQPAGKGLEWIGR | 1 (Ref1)_ | | |
| | IYASGNTNYNPSLKSRVTISVDTSK | Heavy Chain | | |
| | NQFSLKLSSMTAADTAVYYCARDYR | (HC) | | |
| | VAGTYYYYYGLDVWGQGTTVTVSSA | | | |
| | STKGPSVFPLAPSSKSTSGGTAALG | | | |
| | CLVKDYFPEPVTVSWNSGALTSGVH | | | |
| | TFPAVLQSSGLYSLSSVVTVPSSSL | | | |
| | GTQTYICNVNHKPSNTKVDKKVEPK | | | |
| | SCDKTHTCPPCPAPEAAGAPSVFLF | | | |
| | PPKPKDTLMISRTPEVTCVVVDVSH | | | |
| | EDPEVKFNWYVDGVEVHNAKTKPRE | | | |
| | EQYNSTYRVVSVLTVLHQDWLNGKE | | | |
| | YKCKVSNKALPAPIEKTISKAKGQP | | | |
| | REPQVYTLPPSRDELTKNQVSLTCL | | | |
| | VKGFYPSDIAVEWESNGQPENNYKT | | | |
| | TPPVLDSDGSFFLYSKLTVDKSRWQ | | | |
| | QGNVFSCSVMHEALHNHYTQKSLSL | | | |
| | SPGK | | | |
| 769 | QSALTQPASVSGSPGQSITISCTGT | Reference- | | |
| | SSDVGVYDYVSWYQQHPGKAPKLMI | 1 (Ref1)_ | | |
| | YEVSNRPSGVSNRFSGSKSGNTASL | Light Chain | | |
| | TISGLQTEDEADYYCSSYTSRSTWV | (LC) | | |
| | FGGGTKLTVLGQPKAAPSVTLEPPS | | | |
| | SEELQANKATLVCLISDFYPGAVTV | | | |
| | AWKADSSPVKAGVETTTPSKQSNNK | | | |
| | YAASSYLSLTPEQWKSHKSYSCQVT | | | |
| | HEGSTVEKTVAPTECS | | | |
| 770 | EVKLQQSGDELVRPGASVKMSCKASGYTFTD | CLONE_111_ | | |
| | YAMHWVKQSPGQGLEWIGSIIPLIDTTSYNQ | VH | | |
| | KFKGKATLTADKSSNTAYMELSRLTSEDSAV | | | |
| | YYCARLGSRYLEGFDYWGQGVMVTVSS | | | |
| 771 | DIQMTQSPSSMSASLGDTVTINCLASQDIGN | CLONE_111_ | | |
| | YLSWYQQKPGKSPKLMIYGATNLEDGVPSRF | VL | | |
| | SGSGSGSDYSLTINSLGYDDEGIYHCHQYYE | | | |
| | YPLTFGSGTKLEIK | | | |
| 772 | EVKLQQSGDELVRPGASVKMSCKAS | CLONE_111_ | | |
| | GYTFTDYAMHWVKQSPGQGLEWIGS | HC | | |
| | IIPLIDTTSYNQKFKGKATLTADKS | | | |
| | SNTAYMELSRLTSEDSAVYYCARLG | | | |
| | SRYLEGFDYWGQGVMVTVSSAKTTA | | | |
| | PSVYPLAPVCGDTTGSSVTLGCLVK | | | |
| | GYFPEPVTLTWNSGSLSSGVHTFPA | | | |
| | VLQSDLYTLSSSVTVISSTWPSQSI | | | |
| | TCNVAHPASSTKVDKKIEPRGPTIK | | | |
| | PCPPCKCPAPNAAGGPSVFIFPPKI | | | |
| | KDVLMISLSPIVTCVVVDVSEDDPD | | | |
| | VQISWFVNNVEVHTAQTQTHREDYN | | | |
| | STLRVVSALPIQHQDWMSGKEFKCK | | | |
| | VNNKDLGAPIERTISKPKGSVRAPQ | | | |
| | VYVLPPPEEEMTKKQVTLTCMVTDE | | | |
| | MPEDIYVEWTNNGKTELNYKNTEPV | | | |
| | LDSDGSYFMYSKLRVEKKNWVERNS | | | |
| | YSCSVVHEGLHNHHTTKSFSRTPGK | | | |
| 773 | DIQMTQSPSSMSASLGDTVTINCLA | CLONE_111_ | | |
| | SQDIGNYLSWYQQKPGKSPKLMIYG | LC | | |
| | ATNLEDGVPSRFSGSGSGSDYSLTI | | | |
| | NSLGYDDEGIYHCHQYYEYPLTEGS | | | |
| | GTKLEIKRADAAPTVSIFPPSSEQL | | | |
| | TSGGASVVCFLNNFYPKDINVKWKI | | | |
| | DGSERQNGVLNSWTDQDSKDSTYSM | | | |
| | SSTLTLTKDEYERHNSYTCEATHKT | | | |
| | STSPIVKSFNRNEC | | | |
| 100 | GGSISGY | CLONE_1 | Chothia | CDR-H1 |
| 101 | GGSISGYYWS | | AbM | |
| 102 | GYYWS | | Kabat | |
| 103 | SGYYWS | | Contact | |
| 104 | GGSISGYY | | IMGT | |
| 105 | GGSISGY | CLONE_1_ | Chothia | CDR-H1 |
| 106 | GGSISGYYWS | germlined | AbM | |
| 107 | GYYWS | | Kabat | |
| 108 | SGYYWS | | Contact | |
| 109 | GGSISGYY | | IMGT | |
| 110 | GGNGDDY | CLONE_2 | Chothia | CDR-H1 |
| 111 | GFNGDDYAMH | | AbM | |
| 112 | DYAMH | | Kabat | |
| 113 | DDYAMH | | Contact | |
| 114 | GFNGDDYA | | IMGT | |
| 115 | GFNGDDY | CLONE_2_ | Chothia | CDR-H1 |
| 116 | GGNGDDYAMH | germlined | AbM | |
| 117 | DYAMH | | Kabat | |
| 118 | DDYAMH | | Contact | |
| 119 | GGNFDDYA | | IMGT | |
| 120 | GGSISGY | CLONE_3 | Chothia | CDR-H1 |
| 121 | GGSISGYYWS | | AbM | |
| 122 | GYYWS | | Kabat | |
| 123 | SGYYWS | | Contact | |
| 124 | GGSISGYY | | IMGT | |
| 125 | GGSISGY | CLONE_3_ | Chothia | CDR-H1 |
| 126 | GGSISGYYWS | germlined | AbM | |
| 127 | GYYWS | | Kabat | |
| 128 | SGYYWS | | Contact | |
| 129 | GGSISGYY | | IMGT | |
| 130 | GFPFSSY | CLONE_4 | Chothia | CDR-H1 |
| 131 | GFPFSSYGMH | | AbM | |
| 132 | SYGMH | | Kabat | |
| 133 | SSYGMH | | Contact | |
| 134 | GFPFSSYG | | IMGT | |
| 135 | GFPFSSY | CLONE_4_ | Chothia | CDR-H1 |
| 136 | GFPFSSYGMH | germlined | AbM | |
| 137 | SYGMH | | Kabat | |
| 138 | SSYGMH | | Contact | |
| 139 | GFPFSSYG | | IMGT | |
| 140 | FYSGY | CLONE_1 | Chothia | CDR-H2 |
| 141 | YIFYSGYTK | | AbM | |
| 142 | YIFYSGYTKYNPSLKS | | Kabat | |
| 143 | WIGYIFYSGYTK | | Contact | |
| 144 | IFYSGYT | | IMGT | |
| 145 | FYSGY | CLONE_1_ | Chothia | CDR-H2 |
| 146 | YIFYSGYTK | germlined | AbM | |
| 147 | YIFYSGYTKYNPSLKS | | Kabat | |
| 148 | WIGYIFYSGYTK | | Contact | |
| 149 | IFYSGYT | | IMGT | |
| 150 | SWRSGN | CLONE_2 | Chothia | CDR-H2 |
| 151 | GISWRSGNIG | | AbM | |
| 152 | GISWRSGNIGYAGSVKG | | Kabat | |
| 153 | WVSGISWRSGNIG | | Contact | |
| 154 | ISWRSGNI | | IMGT | |
| 155 | SWRSGN | CLONE_2_ | Chothia | CDR-H2 |
| 156 | GISWRSGNIG | germlined | AbM | |
| 157 | GISWRSGNIGYADSVKG | | Kabat | |
| 158 | WVSGISWRSGNIG | | Contact | |
| 159 | ISWRSGNI | | IMGT | |
| 160 | FYSGY | CLONE_3 | Chothia | CDR-H2 |
| 161 | YIFYSGYTK | | AbM | |
| 162 | YIFYSGYTKYNPSLKS | | Kabat | |
| 163 | WIGYIFYSGYTK | | Contact | |
| 164 | IFYSGYT | | IMGT | |
| 165 | FYSGY | CLONE_3_ | Chothia | CDR-H2 |
| 166 | YIFYSGYTK | germlined | AbM | |
| 167 | YIFYSGYTKYNPSLKS | | Kabat | |
| 168 | WIGYIFYSGYTK | | Contact | |
| 169 | IFYSGYT | | IMGT | |
| 170 | WFDGNN | CLONE_4 | Chothia | CDR-H2 |
| 171 | FIWFDGNNKD | | AbM | |
| 172 | FIWFDGNNKDYADSVKG | | Kabat | |
| 173 | WVTFIWFDGNNKD | | Contact | |
| 174 | IWFDGNNK | | IMGT | |
| 175 | WFDGNN | CLONE_4_ | Chothia | CDR-H2 |
| 176 | FIWFDGNNKD | germlined | AbM | |
| 177 | FIWFDGNNKDYADSVKG | | Kabat | |
| 178 | WVTFIWFDGNNKD | | Contact | |
| 179 | IWFDGNNK | | IMGT | |
| 180 | DRWDFDY | CLONE_1 | Chothia | CDR-H3 |
| 181 | DRWDFDY | | AbM | |
| 182 | DRWDFDY | | Kabat | |
| 183 | ARDRWDFD | | Contact | |
| 184 | ARDRWDFDY | | IMGT | |
| 185 | DRWDFDY | CLONE_1_ | Chothia | CDR-H3 |
| 186 | DRWDFDY | germlined | AbM | |
| 187 | DRWDFDY | | Kabat | |
| 188 | ARDRWDFD | | Contact | |
| 189 | ARDRWDFDY | | IMGT | |
| 190 | DKGIGFNWNYEGFDY | CLONE_2 | Chothia | CDR-H3 |
| 191 | DKGIGFNWNYEGFDY | | AbM | |
| 192 | DKGIGFNWNYEGFDY | | Kabat | |
| 193 | AKDKGIGFNWNYEGFD | | Contact | |
| 194 | AKDKGIGFNWNYEGFDY | | IMGT | |
| 195 | DKGIGFNWNYEGFDY | CLONE_2_ | Chothia | CDR-H3 |
| 196 | DKGIGFNWNYEGFDY | germlined | AbM | |
| 197 | DKGIGFNWNYEGFDY | | Kabat | |
| 198 | AKDKGIGENWNYEGFD | | Contact | |
| 199 | AKDKGIGENWNYEGFDY | | IMGT | |
| 200 | DRWDFDY | CLONE_3 | Chothia | CDR-H3 |
| 201 | DRWDFDY | | AbM | |
| 202 | DRWDFDY | | Kabat | |
| 203 | ARDRWDFD | | Contact | |
| 204 | ARDRWDFDY | | IMGT | |
| 205 | DRWDFDY | CLONE_3_ | Chothia | CDR-H3 |
| 206 | DRWDFDY | germlined | AbM | |
| 207 | DRWDFDY | | Kabat | |
| 208 | ARDRWDFD | | Contact | |
| 209 | ARDRWDFDY | | IMGT | |
| 210 | NGVYYGSGAYVDY | CLONE_4 | Chothia | CDR-H3 |
| 211 | NGVYYGSGAYVDY | | AbM | |
| 212 | NGVYYGSGAYVDY | | Kabat | |
| 213 | ARNGVYYGSGAYVD | | Contact | |
| 214 | ARNGVYYGSGAYVDY | | IMGT | |
| 215 | NGVYYGSGAYVDY | CLONE_4_ | Chothia | CDR-H3 |
| 216 | NGVYYGSGAYVDY | germlined | AbM | |
| 217 | NGVYYGSGAYVDY | | Kabat | |
| 218 | ARNGVYYGSGAYVD | | Contact | |
| 219 | ARNGVYYGSGAYVDY | | IMGT | |
| 220 | GFTFSIY | CLONE_59 | Chothia | CDR-H1 |
| 221 | GFTFSIYGMH | | AbM | |
| 222 | IYGMH | | Kabat | |
| 223 | SIYGMH | | Contact | |
| 224 | GFTFSIYG | | IMGT | |
| 225 | GFAFNIY | CLONE_60 | Chothia | CDR-H1 |
| 226 | IFAFNIYGMH | | AbM | |
| 227 | TYGMH | | Kabat | |
| 228 | NIYGMH | | Contact | |
| 229 | GFAFNIYG | | IMGT | |
| 230 | GFSLSTSGV | CLONE_61 | Chothia | CDR-H1 |
| 231 | GFSLSTSGVGVG | | AbM | |
| 232 | TSGVGVG | | Kabat | |
| 233 | STSGVGVG | | Contact | |
| 234 | GFSLSTSGVG | | IMGT | |
| 235 | WYDGSN | CLONE_59 | Chothia | CDR-H2 |
| 236 | VIWYDGSNKY | | AbM | |
| 237 | VIWYDGSNKYYVDSVKG | | Kabat | |
| 238 | WVAVIWYDGSNKY | | Contact | |
| 239 | IWYDGSNK | | IMGT | |
| 240 | SYDGSN | CLONE_60 | Chothia | CDR-H2 |
| 241 | VISYDGSNKV | | AbM | |
| 242 | VISYDGSNKVYADSVKG | | Kabat | |
| 243 | WVAVISYDGSNKV | | Contact | |
| 244 | ISYDGSNK | | IMGT | |
| 245 | YWNDD | CLONE_61 | Chothia | CDR-H2 |
| 246 | LIYWNDDKR | | AbM | |
| 247 | LIYWNDDKRYSPSLKS | | Kabat | |
| 248 | WLALIYWNDDKR | | Contact | |
| 249 | IYWNDDK | | IMGT | |
| 250 | EGNLFDY | CLONE_59 | Chothia | CDR-H3 |
| 251 | EGNLFDY | | AbM | |
| 252 | EGNLFDY | | Kabat | |
| 253 | AREGNLFD | | Contact | |
| 254 | AREGNLFDY | | IMGT | |
| 255 | SGGITMVRGVFDY | CLONE_60 | Chothia | CDR-H3 |
| 256 | SGGITMVRGVFDY | | AbM | |
| 257 | SGGITMVRGVFDY | | Kabat | |
| 258 | AKSGGITMVRGVFD | | Contact | |
| 259 | AKSGGITMVRGVFDY | | IMGT | |
| 260 | RRTTTVTIYYYYMDV | CLONE_61 | Chothia | CDR-H3 |
| 261 | RRTTTVTIYYYYMDV | | AbM | |
| 262 | RRTTTVTIYYYYMDV | | Kabat | |
| 263 | AHRRITTVTIYYYYMD | | Contact | |
| 264 | AHRRITTVTIYYYYMDV | | IMGT | |
| 300 | RASQGIRNNLG | CLONE_1 | Chothia | CDR-L1 |
| 301 | RASQGIRNNLG | | AbM | |
| 302 | RASQGIRNNLG | | Kabat | |
| 303 | RNNLGWY | | Contact | |
| 304 | QGIRNN | | IMGT | |
| 305 | RASQGIRNNLG | CLONE_1_ | Chothia | CDR-L1 |
| 306 | RASQGIRNNLG | germlined | AbM | |
| 307 | RASQGIRNNLG | | Kabat | |
| 308 | RNNLGWY | | Contact | |
| 309 | QGIRNN | | IMGT | |
| 310 | RASQGISNHLA | CLONE_2 | Chothia | CDR-L1 |
| 311 | RASQGISNHLA | | AbM | |
| 312 | RASQGISNHLA | | Kabat | |
| 313 | SNHLAWE | | Contact | |
| 314 | QGISNH | | IMGT | |
| 315 | RASQGISNHLA | CLONE_2_ | Chothia | CDR-L1 |
| 316 | RASQGISNHLA | germlined | AbM | |
| 317 | RASQGISNHLA | | Kabat | |
| 318 | SNHLAWE | | Contact | |
| 319 | QGISNH | | IMGT | |
| 320 | RSSQSLLHSNGYNYLD | CLONE_3 | Chothia | CDR-L1 |
| 321 | RSSQSLLHSNGYNYLD | | AbM | |
| 322 | RSSQSLLHSNGYNYLD | | Kabat | |
| 323 | LHSNGYNYLDWY | | Contact | |
| 324 | QSLLHSNGYNY | | IMGT | |
| 325 | RSSQSLLHSNGYNYLD | CLONE_3_ | Chothia | CDR-L1 |
| 326 | RSSQSLLHSNGYNYLD | germlined | AbM | |
| 327 | RSSQSLLHSNGYNYLD | | Kabat | |
| 328 | LHSNGYNYLDWY | | Contact | |
| 329 | QSLLHSNGYNY | | IMGT | |
| 330 | RSSQSLLHSNGYNYLD | CLONE_4 | Chothia | CDR-L1 |
| 331 | RSSQSLLHSNGYNYLD | | AbM | |
| 332 | RSSQSLLHSNGYNYLD | | Kabat | |
| 333 | LHSNGYNYLDWY | | Contact | |
| 334 | QSLLHSNGYNY | | IMGT | |
| 335 | RSSQSLLHSNGYNYLD | CLONE_4_ | Chothia | CDR-L1 |
| 336 | RSSQSLLHSNGYNYLD | germlined | AbM | |
| 337 | RSSQSLLHSNGYNYLD | | Kabat | |
| 338 | LHSNGYNYLDWY | | Contact | |
| 339 | QSLLHSNGYNY | | IMGT | |
| 340 | AESSLQS | CLONE_1 | Chothia | CDR-L2 |
| 341 | AESSLQS | | AbM | |
| 342 | AESSLQS | | Kabat | |
| 343 | LLIYAESSLQ | | Contact | |
| 344 | AE | | IMGT | |
| 345 | AESSLQS | CLONE_1_ | Chothia | CDR-L2 |
| 346 | AESSLQS | germlined | AbM | |
| 347 | AESSLQS | | Kabat | |
| 348 | LLIYAESSLQ | | Contact | |
| 349 | AE | | IMGT | |
| 350 | AASSLQS | CLONE_2 | Chothia | CDR-L2 |
| 351 | AASSLQS | | AbM | |
| 352 | AASSLQS | | Kabat | |
| 353 | SLIYAASSLQ | | Contact | |
| 354 | AA | | IMGT | |
| 355 | AASSLQS | CLONE_2_ | Chothia | CDR-L2 |
| 356 | AASSLQS | germlined | AbM | |
| 357 | AASSLQS | | Kabat | |
| 358 | SLIYAASSLQ | | Contact | |
| 359 | AA | | IMGT | |
| 360 | LGSNRAS | CLONE_3 | Chothia | CDR-L2 |
| 361 | LGSNRAS | | AbM | |
| 362 | LGSNRAS | | Kabat | |
| 363 | LLIYLGSNRA | | Contact | |
| 364 | LG | | IMGT | |
| 365 | LGSNRAS | CLONE_3_ | Chothia | CDR-L2 |
| 366 | LGSNRAS | germlined | AbM | |
| 367 | LGSNRAS | | Kabat | |
| 368 | LLIYLGSNRA | | Contact | |
| 369 | LG | | IMGT | |
| 370 | LGSNRAS | CLONE_4 | Chothia | CDR-L2 |
| 371 | LGSNRAS | | AbM | |
| 372 | LGSNRAS | | Kabat | |
| 373 | LLIYLGSNRA | | Contact | |
| 374 | LG | | IMGT | |
| 375 | LGSNRAS | CLONE_4_ | Chothia | CDR-L2 |
| 376 | LGSNRAS | germlined | AbM | |
| 377 | LGSNRAS | | Kabat | |
| 378 | LLIYLGSNRA | | Contact | |
| 379 | LG | | IMGT | |
| 380 | LQDFNYPYT | CLONE_1 | Chothia | CDR-L3 |
| 381 | LQDFNYPYT | | AbM | |
| 382 | LQDFNYPYT | | Kabat | |
| 383 | LQDFNYPY | | Contact | |
| 384 | LQDFNYPYT | | IMGT | |
| 385 | LQDFNYPYT | CLONE_1_ | Chothia | CDR-L3 |
| 386 | LQDFNYPYT | germlined | AbM | |
| 387 | LQDFNYPYT | | Kabat | |
| 388 | LQDFNYPY | | Contact | |
| 389 | LQDFNYPYT | | IMGT | |
| 390 | QQYVTYPLT | CLONE_2 | Chothia | CDR-L3 |
| 391 | QQYVTYPLT | | AbM | |
| 392 | QQYVTYPLT | | Kabat | |
| 393 | QQYVTYPL | | Contact | |
| 394 | QQYVTYPLT | | IMGT | |
| 395 | QQYVTYPLT | CLONE_2_ | Chothia | CDR-L3 |
| 396 | QQYVTYPLT | germlined | AbM | |
| 397 | QQYVTYPLT | | Kabat | |
| 398 | QQYVTYPL | | Contact | |
| 399 | QQYVTYPLT | | IMGT | |
| 400 | MQALQTPRT | CLONE_3 | Chothia | CDR-L3 |
| 401 | MQALQTPRT | | AbM | |
| 402 | MQALQTPRT | | Kabat | |
| 403 | MQALQTPR | | Contact | |
| 404 | MQALQTPRT | | IMGT | |
| 405 | MQALQTPRT | CLONE_3_ | Chothia | CDR-L3 |
| 406 | MQALQTPRT | germlined | AbM | |
| 407 | MQALQTPRT | | Kabat | |
| 408 | MQALQTPR | | Contact | |
| 409 | MQALQTPRT | | IMGT | |
| 410 | MQALQTPRT | CLONE_4 | Chothia | CDR-L3 |
| 411 | MQALQTPRT | | AbM | |
| 412 | MQALQTPRT | | Kabat | |
| 413 | MQALQTPR | | Contact | |
| 414 | MQALQTPRT | | IMGT | |
| 415 | MQALQTPRT | CLONE_4_ | Chothia | CDR-L3 |
| 416 | MQALQTPRT | germlined | AbM | |
| 417 | MQALQTPRT | | Kabat | |
| 418 | MQALQTPR | | Contact | |
| 419 | MQALQTPRT | | IMGT | |
| 420 | RASQSISSWLA | CLONE_59 | Chothia | CDR-L1 |
| 421 | RASQSISSWLA | | AbM | |
| 422 | RASQSISSWLA | | Kabat | |
| 423 | SSWLAWY | | Contact | |
| 424 | QSISSW | | IMGT | |
| 425 | RASQGIRNDLG | CLONE_60 | Chothia | CDR-L1 |
| 426 | RASQGIRNDLG | | AbM | |
| 427 | RASQGIRNDLG | | Kabat | |
| 428 | RNDLGWY | | Contact | |
| 429 | QGIRND | | IMGT | |
| 430 | RASQSVSSNLA | CLONE_61 | Chothia | CDR-L1 |
| 431 | RASQSVSSNLA | | AbM | |
| 432 | RASQSVSSNLA | | Kabat | |
| 433 | SSNLAWY | | Contact | |
| 434 | QSVSSN | | IMGT | |
| 435 | KASSLES | CLONE_59 | Chothia | CDR-L2 |
| 436 | KASSLES | | AbM | |
| 437 | KASSLES | | Kabat | |
| 438 | LLIYKASSLE | | Contact | |
| 439 | KA | | IMGT | |
| 440 | AASSLHS | CLONE_60 | Chothia | CDR-L2 |
| 441 | AASSLHS | | AbM | |
| 442 | AASSLHS | | Kabat | |
| 443 | RLIHAASSLH | | Contact | |
| 444 | AA | | IMGT | |
| 445 | GASTRAT | CLONE_61 | Chothia | CDR-L2 |
| 446 | GASTRAT | | AbM | |
| 447 | GASTRAT | | Kabat | |
| 448 | LLIYGASTRA | | Contact | |
| 449 | GA | | IMGT | |
| 450 | QQYKSYYT | CLONE_59 | Chothia | CDR-L3 |
| 451 | QQYKSYYT | | AbM | |
| 452 | QQYKSYYT | | Kabat | |
| 453 | QQYKSYY | | Contact | |
| 454 | QQYKSYYT | | IMGT | |
| 455 | LQHNRYPIT | CLONE_60 | Chothia | CDR-L3 |
| 456 | LQHNRYPIT | | AbM | |
| 457 | LQHNRYPIT | | Kabat | |
| 458 | LQHNRYPI | | Contact | |
| 459 | LQHNRYPIT | | IMGT | |
| 460 | QQYNNWPFT | CLONE_61 | Chothia | CDR-L3 |
| 461 | QQYNNWPFT | | AbM | |
| 462 | QQYNNWPFT | | Kabat | |
| 463 | QQYNNWPF | | Contact | |
| 464 | QQYNNWPFT | | IMGT | |
| 465 | GFMFSSY | CLONE_63 | Chothia | CDR-H1 |
| 466 | GFMFSSYSMN | | AbM | |
| 467 | SYSMN | | Kabat | |
| 468 | SSYSMN | | Contact | |
| 469 | GFMFSSYS | | IMGT | |
| 470 | GFTFSNY | CLONE_64 | Chothia | CDR-H1 |
| 471 | GFTFSNYAMT | | AbM | |
| 472 | NYAMT | | Kabat | |
| 473 | SNYAMT | | Contact | |
| 474 | GFTFSNYA | | IMGT | |
| 475 | GFTFSNY | CLONE_65 | Chothia | CDR-H1 |
| 476 | GFTFSNYAMT | | AbM | |
| 477 | NYAMT | | Kabat | |
| 478 | SNYAMT | | Contact | |
| 479 | GFTFSNYA | | IMGT | |
| 480 | GFTFSSY | CLONE_80 | Chothia | CDR-H1 |
| 481 | GFTFSSYDMH | | AbM | |
| 482 | SYDMH | | Kabat | |
| 483 | SSYDMH | | Contact | |
| 484 | GFTFSSYD | | IMGT | |
| 485 | GITFSNA | CLONE_82 | Chothia | CDR-H1 |
| 486 | GITFSNAWMS | | AbM | |
| 487 | NAWMS | | Kabat | |
| 488 | SNAWMS | | Contact | |
| 489 | GITFSNAW | | IMGT | |
| 490 | SSSSST | CLONE_63 | Chothia | CDR-H2 |
| 491 | YISSSSSTIY | | AbM | |
| 492 | YISSSSSTIYDADSVKG | | Kabat | |
| 493 | WVSYISSSSSTIY | | Contact | |
| 494 | ISSSSSTI | | IMGT | |
| 495 | SGFGGS | CLONE_64 | Chothia | CDR-H2 |
| 496 | AISGFGGSTY | | AbM | |
| 497 | AISGFGGSTYYADSVKG | | Kabat | |
| 498 | WVSAISGFGGSTY | | Contact | |
| 499 | ISGFGGST | | IMGT | |
| 513 | SGYGGS | CLONE_65 | Chothia | CDR-H2 |
| 514 | AISGYGGSTY | | AbM | |
| 515 | AISGYGGSTYYAASVKG | | Kabat | |
| 516 | WVSAISGYGGSTY | | Contact | |
| 517 | ISGYGGST | | IMGT | |
| 518 | GIGGD | CLONE_80 | Chothia | CDR-H2 |
| 519 | SIGIGGDTY | | AbM | |
| 520 | SIGIGGDTYYPGSVKG | | Kabat | |
| 521 | WVSSIGIGGDTY | | Contact | |
| 522 | IGIGGDT | | IMGT | |
| 523 | KSKTYGGT | CLONE_82 | Chothia | CDR-H2 |
| 524 | RIKSKTYGGTTD | | AbM | |
| 525 | RIKSKTYGGTTDYAAPVKG | | Kabat | |
| 526 | WVGRIKSKTYGGTTD | | Contact | |
| 527 | IKSKTYGGTT | | IMGT | |
| 528 | EAYPGYYYNYMDV | CLONE_63 | Chothia | CDR-H3 |
| 529 | EAYPGYYYNYMDV | | AbM | |
| 530 | EAYPGYYYNYMDV | | Kabat | |
| 531 | TREAYPGYYYNYMD | | Contact | |
| 532 | TREAYPGYYYNYMDV | | IMGT | |
| 533 | DHDYYAFDY | CLONE_64 | Chothia | CDR-H3 |
| 534 | DHDYYAFDY | | AbM | |
| 535 | DHDYYAFDY | | Kabat | |
| 536 | AKDHDYYAFD | | Contact | |
| 537 | AKDHDYYAFDY | | IMGT | |
| 538 | DHDYYAFDY | CLONE_65 | Chothia | CDR-H3 |
| 539 | DHDYYAFDY | | AbM | |
| 540 | DHDYYAFDY | | Kabat | |
| 541 | AKDHDYYAFD | | Contact | |
| 542 | AKDHDYYAFDY | | IMGT | |
| 543 | GEWDLLWYFMDV | CLONE_80 | Chothia | CDR-H3 |
| 544 | GEWDLLWYFMDV | | AbM | |
| 545 | GEWDLLWYFMDV | | Kabat | |
| 546 | ARGEWDLLWYFMD | | Contact | |
| 547 | ARGEWDLLWYFMDV | | IMGT | |
| 548 | DPWNYVNYNYFMDV | CLONE_82 | Chothia | CDR-H3 |
| 549 | DPWNYVNYNYFMDV | | AbM | |
| 550 | DPWNYVNYNYFMDV | | Kabat | |
| 551 | TTDPWNYVNYNYFMD | | Contact | |
| 552 | TTDPWNYVNYNYFMDV | | IMGT | |
| 553 | RASQSISSRLA | CLONE_63 | Chothia | CDR-L1 |
| 554 | RASQSISSRLA | | AbM | |
| 555 | RASQSISSRLA | | Kabat | |
| 556 | SSRLAWY | | Contact | |
| 557 | QSISSR | | IMGT | |
| 558 | RASQSISSYLN | CLONE_64 | Chothia | CDR-L1 |
| 559 | RASQSISSYLN | | AbM | |
| 560 | RASQSISSYLN | | Kabat | |
| 561 | SSYLNWY | | Contact | |
| 562 | QSISSY | | IMGT | |
| 563 | RASQSINSYLN | CLONE_65 | Chothia | CDR-L1 |
| 564 | RASQSINSYLN | | AbM | |
| 565 | RASQSINSYLN | | Kabat | |
| 566 | NSYLNWY | | Contact | |
| 567 | QSINSY | | IMGT | |
| 568 | RASQGISSWLA | CLONE_80 | Chothia | CDR-L1 |
| 569 | RASQGISSWLA | | AbM | |
| 570 | RASQGISSWLA | | Kabat | |
| 571 | SSWLAWY | | Contact | |
| 572 | QGISSW | | IMGT | |
| 573 | RASQDIRNYLA | CLONE_82 | Chothia | CDR-L1 |
| 574 | RASQDIRNYLA | | AbM | |
| 575 | RASQDIRNYLA | | Kabat | |
| 576 | RNYLAWY | | Contact | |
| 577 | QDIRNY | | IMGT | |
| 578 | KASNLES | CLONE_63 | Chothia | CDR-L2 |
| 579 | KASNLES | | AbM | |
| 580 | KASNLES | | Kabat | |
| 581 | LLIYKASNLE | | Contact | |
| 582 | KA | | IMGT | |
| 583 | AVSSLQS | CLONE_64 | Chothia | CDR-L2 |
| 584 | AVSSIQS | | AbM | |
| 585 | AVSSLQS | | Kabat | |
| 586 | LLIYAVSSLQ | | Contact | |
| 587 | AV | | IMGT | |
| 588 | SASSLQS | CLONE_65 | Chothia | CDR-L2 |
| 589 | SASSLQS | | AbM | |
| 590 | SASSLQS | | Kabat | |
| 591 | LLIYSASSLQ | | Contact | |
| 592 | SA | | IMGT | |
| 593 | AASSLQS | CLONE_80 | Chothia | CDR-L2 |
| 594 | AASSLQS | | AbM | |
| 595 | AASSLQS | | Kabat | |
| 596 | ILIYAASSLQ | | Contact | |
| 597 | AA | | IMGT | |
| 598 | AASTLQS | CLONE_82 | Chothia | CDR-L2 |
| 599 | AASTLQS | | AbM | |
| 600 | AASTLQS | | Kabat | |
| 601 | LIVYAASTIQ | | Contact | |
| 602 | AA | | IMGT | |
| 603 | QQYNSYSRT | CLONE_63 | Chothia | CDR-L3 |
| 604 | QQYNSYSRT | | AbM | |
| 605 | QQYNSYSRT | | Kabat | |
| 606 | QQYNSYSR | | Contact | |
| 607 | QQYNSYSRT | | IMGT | |
| 608 | QQSYSTPYT | CLONE_64 | Chothia | CDR-L3 |
| 609 | QQSYSTPYT | | AbM | |
| 610 | QQSYSTPYT | | Kabat | |
| 611 | QQSYSTPY | | Contact | |
| 612 | QQSYSTPYT | | IMGT | |
| 613 | QQSYSTPYT | CLONE_65 | Chothia | CDR-L3 |
| 614 | QQSYSTPYT | | AbM | |
| 615 | QQSYSTPYT | | Kabat | |
| 616 | QQSYSTPY | | Contact | |
| 617 | QQSYSTPYT | | IMGT | |
| 618 | QQANSFPLT | CLONE_80 | Chothia | CDR-L3 |
| 619 | QQANSEPIT | | AbM | |
| 620 | QQANSFPLT | | Kabat | |
| 621 | QQANSFPL | | Contact | |
| 622 | QQANSFPLT | | IMGT | |
| 623 | QNYFSVPLT | CLONE_82 | Chothia | CDR-L3 |
| 624 | QNYFSVPLT | | AbM | |
| 625 | QNYFSVPLT | | Kabat | |
| 626 | QNYFSVPL | | Contact | |
| 627 | QNYFSVPLT | | IMGT | |
| 628 | VSGGSISGYY | CLONE_1_ | Aho | CDR-H1 |
| | | germlined | | |
| 629 | ASGFNFDDYA | CLONE_2_ | Aho | CDR-H1 |
| | | geimlined | | |
| 630 | VSGFTFSSYD | CLONE_80 | Ahc | CDR-H1 |
| 631 | ASGFMFSSYS | CLONE_63 | Aho | CDR-H1 |
| 632 | ASGFTFSNYA | CLONE_64 | Aho | CDR-H1 |
| 633 | ASGFTFSNYA | CLONE_65 | Aho | CDR-H1 |
| 634 | VSGITFSNAW | CLONE_82 | Aho | CDR-H1 |
| 635 | GYYWS (SEQ ID NO: 107) | CLONE_1_ | Cross- | CDR-H1 |
| | | germlined | linking | |
| 636 | DYAMH (SEQ ID NO: 117) | CLONE_2_ | Cross- | CDR-H1 |
| | | germlined | linking | |
| 637 | SYDMH (SEQ ID NO: 482) | CLONE_80 | Cross- | CDR-H1 |
| | | | linking | |
| 638 | SYSMN (SEQ ID NO: 467) | CIONE_63 | Cross- | CDR-H1 |
| | | | linking | |
| 639 | NYAMT (SEQ ID NO: 472) | CLONE_64 | Cross- | CDR-H1 |
| | | | linking | |
| 640 | NYAMT (SEQ ID NO: 477) | CLONE_65 | Cross- | CDR-H1 |
| | | | linking | |
| 641 | NAWMS (SEQ ID NO: 487) | CIONE_82 | Cross- | CDR-H1 |
| | | | linking | |
| 642 | IFYSGYTKYNPSLKSR | CLONE_1_ | Aho | CDR-H2 |
| | | germlined | | |
| 643 | ISWRSGNIGYADSVKGR | CLONE_2_ | Aho | CDR-H2 |
| | | germlined | | |
| 644 | IGIGGDTYYPGSVKGR | CLONE_80 | Aho | CDR-H2 |
| 645 | ISSSSSTIYDADSVKGR | CLONE_63 | Aho | CDR-H2 |
| 646 | ISGFGGSTYYADSVKGR | CLONE_64 | Aho | CDR-H2 |
| 647 | ISGYGGSTYYAASVKGR | CLONE_65 | Aho | CDR-H2 |
| 648 | IKSKTYGGTTDYAAPVKGR | CLONE_82 | Aho | CDR-H2 |
| 649 | WIGYIFYSGYTK (SEQ ID NO: | CLONE_1_ | Cross- | CDR-H2 |
| | 148) | germlined | linking | |
| 650 | WVSGISWRSGNIG (SEQ ID NO: | CLONE_2_ | Cross- | CDR-H2 |
| | 158) | germlined | linking | |
| 651 | WVSSIGIGGDTY (SEQ ID NO: | CLONE_80 | Cross- | CDR-H2 |
| | 521) | | linking | |
| 652 | WVSYISSSSSTIY (SEQ ID NO: | CLONE_63 | Cross- | CDR-H2 |
| | 493) | | linking | |
| 653 | WVSAISGFGGSTY (SEQ ID NO: | CLONE_64 | Cross- | CDR-H2 |
| | 498) | | linking | |
| 654 | WVSAISGYGGSTY (SEQ ID NO: | CLONE_65 | Cross- | CDR-H2 |
| | 516) | | linking | |
| 655 | WVGRIKSKTYGGTTD (SEQ ID | CLONE_82 | Cross- | CDR-H2 |
| | NO: 526) | | linking | |
| 656 | DRWDFD | CLONE_1_ | Aho | CDR-H3 |
| | | germlined | | |
| 657 | DKGIGENWNYEGFD | CLONE_2_ | Aho | CDR-H3 |
| | | germlined | | |
| 658 | GEWDLLWYFMD | CLONE_80 | Aho | CDR-H3 |
| 659 | EAYPGYYYNYMD | CLONE_63 | Aho | CDR-H3 |
| 660 | DHDYYAFD | CLONE_64 | Ahc | CDR-H3 |
| 661 | DHDYYAFD | CLONE_65 | Aho | CDR-H3 |
| 662 | DPWNYVNYNYFMD | CLONE_82 | Aho | CDR-H3 |
| 663 | DRWDFDY (SEQ ID NO: 187) | CLONE_1_ | Cross- | CDR-H3 |
| | | germlined | linking | |
| 664 | DKGIGFNWNYEGFDY (SEQ ID | CLONE_2_ | Cross- | CDR-H3 |
| | NO: 197) | germlined | linking | |
| 665 | GEWDLLWYFMDV (SEQ ID NO: | CLONE_80 | Cross- | CDR-H3 |
| | 545) | | linking | |
| 666 | EAYPGYYYNYMDV (SEQ ID NO: | CLONE_63 | Cross- | CDR-H3 |
| | 530) | | linking | |
| 667 | DHDYYAFDY (SEQ ID NO: | CLONE_64 | Cross- | CDR-H3 |
| | 535) | | linking | |
| 668 | DHDYYAFDY (SEQ ID NO: | CLONE_65 | Cross- | CDR-H3 |
| | 540) | | linking | |
| 669 | DPWNYVNYNYFMDV (SEQ ID | CLONE_82 | Cross- | CDR-H3 |
| | NO: 550) | | linking | |
| 670 | ASQGIRNN | CLONE_1_ | Aho | CDR-11 |
| | | germlined | | |
| 671 | ASQGISNH | CLONE_2_ | Aho | CDR-11 |
| | | germlined | | |
| 672 | ASQGISSW | CLONE_80 | Aho | CDR-L1 |
| 673 | ASQSISSR | CLONE_63 | Aho | CDR-11 |
| 674 | ASQSISSY | CLONE_64 | Aho | CDR-L1 |
| 675 | ASQSINSY | CLONE_65 | Aho | CDR-11 |
| 676 | ASQDIRNY | CLONE_82 | Aho | CDR-IL |
| 677 | RASQGIRNNIG (SEQ ID NO: | CLONE_1_ | Cross- | CDR-11 |
| | 307) | germlined | linking | |
| 678 | RASQGISNHLA (SEQ ID NO: | CLONE_2_ | Cross- | CDR-11 |
| | 317) | germlined | linking | |
| 679 | RASQGISSWLA (SEQ ID NO: | CLONE_80 | Cross- | CDR-11 |
| | 570) | | linking | |
| 680 | RASQSISSRLA (SEQ ID NO: | CLONE_63 | Cross- | CDR-11 |
| | 555) | | linking | |
| 681 | RASQSISSYIN (SEQ ID NO: | CLONE_64 | Cross- | CDR-11 |
| | 560) | | linking | |
| 682 | RASQSINSYLN (SEQ ID NO: | CLONE_65 | Cross- | CDR-L1 |
| | 565) | | linking | |
| 683 | RASQDIRNYLA (SEQ ID NO: | CLONE_82 | Cross- | CDR-L1 |
| | 575) | | linking | |
| 684 | AESSLQSGVPSR | CLONE_1_ | Aho | CDR-1:2 |
| | | germlined | | |
| 685 | AASSLQSGVPSR | CLONE_2_ | Aho | CDR-L2 |
| | | germlined | | |
| 686 | AASSLQSGVPSR | CLONE_80 | Aho | CDR-12 |
| 687 | KASNLESGVPSR | CLONE_63 | Aho | CDR-12 |
| 688 | AVSSLQSGVPSR | CLONE_64 | Aho | CDR-12 |
| 689 | SASSLQSGVPSR | CLONE_65 | Aho | CDR-L2 |
| 690 | AASTLQSGVPSR | CLONE_82 | Aho | CDR-12 |
| 691 | AESSLQS (SEQ ID NO: 347) | CLONE_1_ | Cross- | CDR-L2 |
| | | germlined | linking | |
| 692 | AASSLQS (SEQ ID NO: 357) | CLONE_2_ | Cross~ | CDR-12 |
| | | germlined | linking | |
| 693 | AASSLQS (SEQ ID NO: 595) | CLONE_80 | Cross- | CDR-L2 |
| | | | linking | |
| 694 | KASNLES (SEQ ID NO: 580) | CLONE_63 | Cross- | CDR-12 |
| | | | linking | |
| 695 | AVSSLQS (SEQ ID NO: 585) | CLONE_64 | Cross- | CDR-12 |
| | | | linking | |
| 696 | SASSLQS (SEQ ID NO: 590) | CLONE_65 | Cross- | CDR-L2 |
| | | | linking | |
| 697 | AASTIQS (SEQ ID NO: 600) | CLONE_82 | Cross- | CDR-12 |
| | | | linking | |
| 698 | DFNYPY | CLONE_1_ | Aho | CDR-13 |
| | | germlined | | |
| 699 | YVTYPL | CLONE_2_ | Ahc | CDR-L3 |
| | | germlined | | |
| 700 | ANSFPL | CLONE_80 | Aho | CDR-L3 |
| 701 | YNSYSR | CLONE_63 | Aho | CDR-13 |
| 702 | SYSTPY | CLONE_64 | Aho | CDR-13 |
| 703 | SYSTPY | CLONE_65 | Aho | CDR-13 |
| 704 | YESVPL | CLONE_82 | Aho | CDR-L3 |
| 705 | IQDFNYPYT (SEQ ID NO: | CLONE_1_ | Cross- | CDR-13 |
| | 387) | germlined | linking | |
| 706 | QQYVTYPLT (SEQ ID NO: | CLONE_2_ | Cross- | CDR-13 |
| | 397) | germlined | linking | |
| 707 | QQANSFPLT (SEQ ID NO: | CLONE_80 | Cross- | CDR-13 |
| | 620) | | linking | |
| 708 | QQYNSYSRT (SEQ ID NO: | CLONE_63 | Cross- | CDR-13 |
| | 605) | | linking | |
| 709 | QQSYSTPYT (SEQ ID NO: | CLONE_64 | Cross- | CDR-13 |
| | 610) | | linking | |
| 710 | QQSYSTPYT (SEQ ID NO: | CLONE_65 | Cross- | CDR-L3 |
| | 615) | | linking | |
| 711 | QNYESVPLT (SEQ ID NO: | CLONE_82 | Cross- | CDR-L3 |
| | 625) | | linking | |
| 712 | X1-Y-X2-X3-X4, wherein | Consensus_ | based on | CDR-H1 |
| | X1 is G, D, or S; X2 is | 1 | Kabat | |
| | Y, A, or D; X3 is W or M; | | | |
| | and X4 is S or H (SEQ ID | | | |
| | NO: 712) | | | |
| 713 | X1-X2-X3-X4-X5, wherein | Consensus_ | based on | CDR-H1 |
| | X1 is G, D, N, or S; X2 | 2 | Kabat | |
| | is Y or A; X3 is Y, A, S, | | | |
| | W, or D; X4 is W or M; | | | |
| | and X5 is N, T, S or H | | | |
| | (SEQ ID NO: 713) | | | |
| 714 | G-X1-X2-X3-X4-X5-Y, | Consensus_ | based on | CDR-H1 |
| | wherein X1 is G or F; X2 | 1 | Chothia | |
| | is S, N, or T; X3 is I or | | | |
| | F; X4 is S of D; and X5 | | | |
| | is G, D, or S (SEQ ID NO: | | | |
| | 714) | | | |
| 715 | G-X1-X2-X3-X4-X5-X6, | Consensus_ | based on | CDR-H1 |
| | wherein X1 is G, E or | 2 | Chothia | |
| | I; X2 is S, N, T, or M; | | | |
| | X3 is I or F; X4 is S or | | | |
| | D; X5 is G, D, S, or N; | | | |
| | and X6 is Y or A (SEQ ID | | | |
| | NO: 715) | | | |
| 716 | G-X1-X2-X3-X4-X5-Y-X6-X7- | Consensus_ | based on | CDR-H1 |
| | X8, wherein X1 is G or | 1 | AbM | |
| | F; X2 is S, N, or T; X3 | | | |
| | is I or F; X4 is S or D; | | | |
| | X5 is G, D, or S; X6 is | | | |
| | Y, A, or D; X7 is W or M; | | | |
| | and X8 is S or H (SEQ ID | | | |
| | NO: 716) | | | |
| 717 | G-X1-X2-X3-X4-X5-X6-X7- | Consensus_ | based on | CDR-H1 |
| | X8-X9, wherein X1 is G, | 2 | AbM | |
| | F or I; X2 is S, N, T, or | | | |
| | M; X3 is I or E; X4 is S | | | |
| | or D; X5 is G, D, S, or | | | |
| | N; X6 is Y or A; X7 is | | | |
| | Y, A, D, S or W; X8 is W | | | |
| | or M; and X9 is S, H, N, | | | |
| | or T (SEQ ID NO: 717) | | | |
| 718 | X1-X2-Y-X3-X4-X5, wherein | Consensus_ | based on | CDR-H1 |
| | X1 is S or D; X2 is G, D, | 1 | Contact | |
| | or S; X3 is Y, A, or D, | | | |
| | X4 is W or M; and X5 is S | | | |
| | or H (SEQ ID NO: 718) | | | |
| 719 | X1-X2-X3-X4-X5-X6, | Consensus_ | based on | CDR-H1 |
| | wherein X1 is S or D; X2 | 2 | Contact | |
| | is G, D, N, or S; X3 is Y | | | |
| | or A; X4 is Y, A, S, W, | | | |
| | or D; X5 is W or M; and | | | |
| | X6 is N, T, S or H (SEQ | | | |
| | ID NO: 719) | | | |
| 720 | G-X1-X2-X3-X4-X5-Y-X6, | Consensus_ | based on | CDR-H1 |
| | wherein X1 is G or F; X2 | 1 | IMGT | |
| | is S, N, or T; X3 is I or | | | |
| | F; X4 is S or D; X5 is | | | |
| | G, D, or S; and X6 is Y, | | | |
| | A, or D (SEQ ID NO: 720) | | | |
| 721 | G-X1-X2-X3-X4-X5-X6-X7, | Consensus_ | based on | CDR-H1 |
| | wherein X1 is G, F or | 2 | IMGT | |
| | I; X2 is S, N, T, or M; | | | |
| | X3 is I or F; X4 is S or | | | |
| | D; X5 is G, D, S, or N; | | | |
| | X6 is Y or A; and X7 is | | | |
| | Y, A, D, S, Or W (SEQ ID | | | |
| | NO: 721) | | | |
| 722 | X1-S-G-X2-X3-X4-X5-X6-Y- | Consensus_ | based on | CDR-H1 |
| | X7, wherein X1 is V or | 1 | Aho | |
| | A; X2 is G or F; X3 is S, | | | |
| | N, or T; X4 is I or F; | | | |
| | X5 is S or D; X6 is G, D, | | | |
| | or S; and X7 is Y, A, or | | | |
| | D (SEQ ID NO: 722) | | | |
| 723 | X1-S-G-X2-X3-X4-X5-X6-X7- | Consensus_ | based on | CDR-H1 |
| | X8, wherein X1 is V or | 2 | Aho | |
| | A; X2 is G, F or I; X3 | | | |
| | is S, N, T, or M; X4 is I | | | |
| | or E; X5 is S or D; X6 | | | |
| | is G, D, S, or N; X7 is | | | |
| | Y or A; and X8 is Y, A; | | | |
| | D, S or W (SEQ ID NO: | | | |
| | 723) | | | |
| 712 | X1-Y-X2-X3-X4, wherein | Consensus_ | based on | CDR-H1 |
| | X1 is G, D, or S; X2 is | 1 | Cross- | |
| | Y, A, or D; X3 is W or M; | | linking | |
| | and X4 is S or H (SEQ ID | | | |
| | NO: 712) | | | |
| 713 | X1-X2-X3-X4-X5, wherein | Consensus_ | based on | CDR-H1 |
| | X1 is G, D, N, or S; X2 | 2 | Cross- | |
| | is Y or A; X3 is Y, A, S, | | linking | |
| | W, or D; X4 is W or M; | | | |
| | and X5 is N, T, S or H | | | |
| | (SEQ ID NO: 713) | | | |
| 724 | X1-I-X2-X3-X4-X5-G-X6- | Consensus_ | based on | CDR-H2 |
| | X7-X8-Y-X9-X10-S-X11-K- | 1 | Kabat | |
| | X12, wherein X1 is Y, G; | | | |
| | or S; X2 is S, G, of | | | |
| | none; X3 is F, W, or I; | | | |
| | X4 is Y, R, or none; X5 | | | |
| | is G ox S; X6 is Y, N, or | | | |
| | D; X7 is T or I; X8 is K, | | | |
| | G, or Y; X9 is N, A, OI | | | |
| | P; and X10 is P, D, or G; | | | |
| | X11 is I or V; and X12 is | | | |
| | S or G (SEQ ID NO: 724) | | | |
| 725 | X1-I-X2-X3-X4-X5-X6- | Consensus_ | based on | CDR-H2 |
| | X7-X8-X9-X10-X11-X12-X13- | 2 | Kabat | |
| | X14-K-X15, wherein X1 is | | | |
| | Y, G, S, A or R; X2 is S, | | | |
| | G, or none; X3 is F, W, | | | |
| | I, S, G or T; X4 is Y, R; | | | |
| | S, F, or none; X5 is G or | | | |
| | S; X6 is G or S; X7 is Y, | | | |
| | N, D, T or S; X8 is T or | | | |
| | I; X9 is K, G, Y, or D; | | | |
| | X10 is Y or D; X11 is N, | | | |
| | A, or P; and X12 is P, D, | | | |
| | G, or A; X13 is S or P; | | | |
| | X14 is L or V; and X15 is | | | |
| | S or G (SEQ ID NO: 725) | | | |
| 726 | X1-X2-X3-X4-G-X5, | Consensus_ | based on | CDR-H2 |
| | wherein X1 is S, G, of | 1 | Chothia | |
| | none; X2 is F, W, or I; | | | |
| | X3 is Y, R, or none; X4 | | | |
| | is S of G; and X5 is Y, | | | |
| | N, or D (SEQ ID NO: 726) | | | |
| 727 | X1-X2-X3-X4-X5-X6, | Consensus_ | based on | CDR-H2 |
| | wherein X1 is S, G, or | 2 | Chothia | |
| | none; X2 is F, W, G, S, | | | |
| | I, or T; X3 is Y, R, S, | | | |
| | F, or none; X4 is S or G; | | | |
| | X5 is G or S) and X6 is | | | |
| | Y, N, D, T, Or S (SEQ ID | | | |
| | NO: 727) | | | |
| 728 | X1-I-X2-X3-X4-X5-G-X6- | Consensus_ | based on | CDR~H2 |
| | X7-X8, wherein X1 is Y, | 1 | AbM | |
| | G, or S; X2 is S, G, or | | | |
| | none; X3 is F, W, or I; | | | |
| | X4 is Y, R, or none; X5 | | | |
| | is G or S; X6 is Y, N, or | | | |
| | D; X7 is T or I; X8 is K, | | | |
| | G, or Y (SEQ ID NO: 728) | | | |
| 729 | X1-I-X2-X3-X4-X5-X6- | Consensus_ | based on | CDR-H2 |
| | X7-X8-X9, wherein X1 is | 2 | AbM | |
| | Y, G, S, A or R; X2 is S, | | | |
| | G, or none; X3 is E, W, | | | |
| | I, S, G or T; X4 is Y, R, | | | |
| | S, F, or none; X5 is G or | | | |
| | S; X6 is G or S; X7 is Y, | | | |
| | N, D, T or S; X8 is T or | | | |
| | I; X9 is K, G, Y, or D | | | |
| | (SEQ ID NO: 729) | | | |
| 730 | W-X1-X2-X3-I-X4-X5-X6-X7- | Consensus_ | based on | CDR-H2 |
| | G-X8-X9-X10, wherein X1 | 1 | Contact | |
| | is I or V; X2 is G or S; | | | |
| | X3 is Y, G, or S; X4 is | | | |
| | S, G, or none; X5 is F, | | | |
| | W, or I; X6 is Y, R, or | | | |
| | none; X7 is G or S; X8 is | | | |
| | Y, N, or D; X9 is T or I; | | | |
| | X 10 is K, G, or Y (SEQ | | | |
| | ID NO: 730) | | | |
| 731 | W-X1-X2-X3-I-X4-X5-X6-X7- | Consensus_ | based on | CDR-H2 |
| | X8-X9-X10-X11, wherein X1 | 2 | Contact | |
| | is I or V; X2 is G or S; | | | |
| | X3 is Y, G, S, A or R; X4 | | | |
| | is S, G, or none; X5 is | | | |
| | F, W, I, S, G or T; X6 is | | | |
| | Y, R, S, F, or none; X7 | | | |
| | is G or S; X8 is G or S; | | | |
| | X9 is Y, N, D, T or S; | | | |
| | X10 is T or I; X 10 is K, | | | |
| | G, Y, or D (SEQ ID NO: | | | |
| | 731) | | | |
| 732 | I-X1-X2-X3-X4-G-X5-X6, | Consensus_ | based on | CDR-H2 |
| | wherein X1 is S, G, or | 1 | IMGT | |
| | none; X2 is F, W, or I; | | | |
| | X3 is Y, R, or none; X4 | | | |
| | is G ox S; X5 is Y, N, or | | | |
| | D; and X6 is T or I (SEQ | | | |
| | ID NO: 732) | | | |
| 733 | I-X1-X2-X3-X4-X5-X6-X7, | Consensus_ | based on | CDR-H2 |
| | wherein X1 is S, G, or | 2 | IMGT | |
| | none; X2 is F, W, I, S, G | | | |
| | or T; X3 is Y, R, S, F, | | | |
| | or none; X4 is G or S; X5 | | | |
| | is G or S; X6 is Y, N, D, | | | |
| | T or S; and X7 is T or I | | | |
| | (SEQ ID NO: 733) | | | |
| 734 | I-X1-X2-X3-X4-G-X5-X6-X7- | Consensus_ | based on | CDR-H2 |
| | Y-X8-X9-S-X10-K-X11-R, | 1 | Aho | |
| | wherein X1 is S, G, or | | | |
| | none; X2 is F, W, or I; | | | |
| | X3 is Y, R, or none; X4 | | | |
| | is G or S; X5 is Y, N, or | | | |
| | D; X6 is T or I; X7 is K, | | | |
| | G, or Y; X8 is N, A, or | | | |
| | P; X9 is P, D or G; X10 | | | |
| | is L or V; and X11 is G or | | | |
| | S (SEQ ID NO: 734) | | | |
| 735 | I-X1-X2-X3-X4-X5-X6-X7- | Consensus_ | based on | CDR-H2 |
| | X8-X9-X10-X11-X12-X13-K- | 2 | Aho | |
| | X14-R, wherein X1 is S, | | | |
| | G, or none; X2 is E, W, | | | |
| | I, S, G or T; X3 is Y, R; | | | |
| | S, F, or none; X4 is G or | | | |
| | S; X5 is G or S; X6 is Y, | | | |
| | N, D, T or S; X7 is T or | | | |
| | I; X8 is K, G, Y, or D; | | | |
| | X9 is Y or D; X10 is N, | | | |
| | A, or P; X11 is P, D, G, | | | |
| | or A; X12 is S or P; X13 | | | |
| | is L or V; and X14 is G | | | |
| | ox S (SEQ ID NO: 735) | | | |
| 730 | W-X1-X2-X3-I-X4-X5-X6-X7- | Consensus_ | based on | CDR-H2 |
| | G-X8-X9-X10, wherein X1 | 1 | Cross- | |
| | is I or V; X2 is G or S; | | linking | |
| | X3 is Y, G, or S; X4 is | | | |
| | S, G, or none; X5 is E, | | | |
| | W, or I; X6 is Y, R, or | | | |
| | none; X7 is G or S; X8 is | | | |
| | Y, N, or D; X9 is T or I; | | | |
| | X 10 is K, G, or Y (SEQ | | | |
| | ID NO: 730) | | | |
| 731 | W-X1-X2-X3-I-X4-X5-X6-X7- | Consensus_ | based on | CDR-H2 |
| | X8-X9-X10-X11, wherein X1 | 2 | Cross- | |
| | is I or V; X2 is G or S; | | linking | |
| | X3 is Y, G, S, A or R; X4 | | | |
| | is S, G, or none; X5 is | | | |
| | F, W, I, S, G or T; X6 is | | | |
| | Y, R, S, E, or none; X7 | | | |
| | is G or S; X8 is G or S; | | | |
| | X9 is Y, N, D, T or S; | | | |
| | X10 is T or I; X10 is K, | | | |
| | G, Y, or D (SEQ ID NO: | | | |
| | 731) | | | |
| 736 | X1-X2-X3-X4-X5-X6-X7-W- | Consensus_ | based on | CDR-H3 |
| | X8-X9-X10-X11-F-X12-D- | 1 | Kabat | |
| | X13, wherein X1 is D or | | | |
| | none; X2 is R, K, or G; | | | |
| | X3 is G, E, or none; X4 | | | |
| | is I, W, or none; X5 is | | | |
| | G, D, or none; X6 is F, | | | |
| | I, or none; X7 is N, L, | | | |
| | or none; X8 is N, Y, or | | | |
| | none; X9 is Y, or none; | | | |
| | X10 is D, E, or none; X11 | | | |
| | is G or none; X12 is M or | | | |
| | none; and X13 is Y ox V | | | |
| | (SEQ ID NO: 736) | | | |
| 737 | X1-X2-X3-X4-X5-X6-X7-X8- | Consensus_ | based on | CDR-H3 |
| | X9-X10-X11-X12-X13-X14-D- | 2 | Kabat | |
| | X15, wherein X1 is D or | | | |
| | none; X2 is R, K, G, P, | | | |
| | or none; X3 is G, E, W, | | | |
| | or none; X4 is I, W, A, | | | |
| | N, or none; X5 is G, D, | | | |
| | Y, or none; X6 is F, | | | |
| | H, V, or none; X7 is | | | |
| | N, L, G, D, or none; X8 | | | |
| | is W or Y; X9 is N, Y, or | | | |
| | none; X10 is Y, A, or | | | |
| | none; X11 is D, E, N, or | | | |
| | none; X12 is G or none; | | | |
| | X13 is F or Y; X14 is M | | | |
| | or none; and X15 is Y or | | | |
| | V (SEQ ID NO: 737) | | | |
| 736 | X1-X2-X3-X4-X5-X6-X7-W- | Consensus_ | based on | CDR-H3 |
| | X8-X9-X10-X11-F-X12-D- | 1 | Chothia | |
| | X13, wherein X1 is D or | | | |
| | none; X2 is R, K, or G; | | | |
| | X3 is G, E, or none; X4 | | | |
| | is I, W, or none; X5 is | | | |
| | G, D, or none; X6 is | | | |
| | L, or none; X7 is N, L, | | | |
| | or none; X8 is N, Y, or | | | |
| | none; X9 is Y, or none; | | | |
| | X10 is D, E, or none; X11 | | | |
| | is G or none; X12 is M or | | | |
| | none; and X13 is Y of V | | | |
| | (SEQ ID NO: 736) | | | |
| 737 | X1-X2-X3-X4-X5-X6-X7-X8- | Consensus_ | based on | CDR-H3 |
| | X9-X10-X11-X12-X13-X14-D- | 2 | Chothia | |
| | X15, wherein X1 is D or | | | |
| | none; X2 is R, K, G, P, | | | |
| | or none; X3 is G, E, W, | | | |
| | or none; X4 is I, W, A, | | | |
| | N, or none; X5 is G, D, | | | |
| | Y, or none; X6 is F, | | | |
| | P, H, V, or none; X7 is | | | |
| | N, L, G, D, or none; X8 | | | |
| | is W or Y; X9 is N, Y, or | | | |
| | none; X10 is Y, A, or | | | |
| | none; X11 is D, E, N, or | | | |
| | none; X12 is G or none; | | | |
| | X13 is F or Y; X14 is M | | | |
| | or none; and X15 is Y or | | | |
| | V (SEQ ID NO: 737) | | | |
| 736 | X1-X2-X3-X4-X5-X6-X7-W- | Consensus_ | based on | CDR-H3 |
| | X8-X9-X10-X11-F-X12-D- | 1 | AbM | |
| | X13, wherein X1 is D or | | | |
| | none; X2 is R, K, or G; | | | |
| | X3 is G, E, or none; X4 | | | |
| | is I, W, or none; X5 is | | | |
| | G, D, or none; X6 is | | | |
| | L, or none; X7 is N, L, | | | |
| | or none; X8 is N, Y, or | | | |
| | none; X9 is Y, or none; | | | |
| | X10 is D, E, of none; X11 | | | |
| | is G or none; X12 is M or | | | |
| | none; and X13 is Y or V | | | |
| | (SEQ ID NO: 736) | | | |
| 737 | X1-X2-X3-X4-X5-X6-X7-X8- | Consensus_ | based on | CDR-H3 |
| | X9-X10-X11-X12-X13-X14-D- | 2 | AbM | |
| | X15, wherein X1 is D or | | | |
| | none; X2 is R, K, G, P, | | | |
| | or none; X3 is G, | | | |
| | or none; X4 is I, W, A, | | | |
| | N, or none; X5 is G, D, | | | |
| | Y, or none; X6 is F, | | | |
| | P, H, V, or none; X7 is | | | |
| | L, G, D, or none; X8 | | | |
| | is W or Y; X9 is N, Y, or | | | |
| | none; X10 is Y, A, or | | | |
| | none; X11 is D, E, N, Or | | | |
| | none; X12 is G or none; | | | |
| | X13 is F or Y; X14 is M | | | |
| | of none; and X15 is Y or | | | |
| | V (SEQ ID NO: 737) | | | |
| 738 | A-X1-X2-X3-X4-X5-X6-X7- | Consensus_ | based on | CDR-H3 |
| | X8-W-X9-X10-X11-X12-F- | 1 | Contact | |
| | X13-D, wherein X1 is K or | | | |
| | R; X2 is D or none; X3 is | | | |
| | R, K, or G; X4 is G, E, | | | |
| | or none; X5 is I, W, or | | | |
| | none; X6 is G, D; or | | | |
| | none; X7 is F, L, or | | | |
| | none; X8 is N, L, or | | | |
| | none; X9 is N, Y, or | | | |
| | none; X10 is Y, or none; | | | |
| | X11 is D, E, or none; X12 | | | |
| | is G or none; and X13 is | | | |
| | M or none (SEQ ID NO; | | | |
| | 738) | | | |
| 739 | X1-X2-X3-X4-X5-X6-X7-X8- | Consensus_ | based on | CDR-H3 |
| | X9-X10-X11-X12-X13-X14- | 2 | Contact | |
| | X15-X16-D, wherein X1 is | | | |
| | A or T; X2 is K, R, or T; | | | |
| | X3 is D or none; X4 is R, | | | |
| | K, G, P, or none; X5 is | | | |
| | G, E, W, or none; X6 is | | | |
| | I, W, A, N, or none; X7 | | | |
| | is G, D, or none; X8 | | | |
| | is F, L, P, H, V, or | | | |
| | none; X9 is N, L, G, D, | | | |
| | or none; X10 is W or Y; | | | |
| | X11 is N, Y, or none; X12 | | | |
| | is Y, A, or none; X13 is | | | |
| | D, E, N, or none; X14 is | | | |
| | G or none; X15 is F or Y; | | | |
| | and X16 is M or none (SEQ | | | |
| | ID NO: 739) | | | |
| 740 | A-X1-X2-X3-X4-X5-X6-X7- | Consensus_ | based on | CDR-H3 |
| | X8-W-X9-X10-X11-X12-F- | 1 | IMGT | |
| | X13-D-X14, wherein X1 is | | | |
| | K or R; X2 is D or none; | | | |
| | X3 is R, K, or G; X4 is | | | |
| | G, E, or none; X5 is I, | | | |
| | W, or none; X6 is G, D, | | | |
| | or none; X7 is F, L, or | | | |
| | none; X8 is N, L, or | | | |
| | none; X9 is N, Y, or | | | |
| | none; X10 is Y, or none; | | | |
| | X11 is D, E, or none; X12 | | | |
| | is G or none; X13 is M or | | | |
| | none; and X14 is Y or V | | | |
| | (SEQ ID NO: 740) | | | |
| 741 | X1-X2-X3-X4-X5-X6-X7-X8- | Consensus_ | based on | CDR-H3 |
| | X9-X10-X11-X12-X13-X14- | 2 | IMGT | |
| | X15-X16-D-X17, wherein X1 | | | |
| | is A or T; X2 is K, R, or | | | |
| | T; X3 is D or none; X4 is | | | |
| | R, K, G, P; or none; X5 | | | |
| | is G, E, W, none; X6 | | | |
| | is I, A, N; or none; | | | |
| | X7 is G, D, or none; | | | |
| | X8 is F, H, V, or | | | |
| | none; X9 is N, L, G, D, | | | |
| | or none; X10 is W or Y; | | | |
| | X11 is N, Y, or none; X12 | | | |
| | is Y, A, or none; X13 is | | | |
| | D, E, N, or none; X14 is | | | |
| | G or none; X15 is F or Y; | | | |
| | X16 is M or none; and X17 | | | |
| | is Y or V (SEQ ID NO: | | | |
| | 741) | | | |
| 742 | X1-X2-X3-X4-X5-X6-X7-W- | Consensus_ | based on | CDR-H3 |
| | X8-X9-X10-X11-F-X12-D, | 1 | Aho | |
| | wherein X1 is D or none; | | | |
| | X2 is R, K, or G; X3 is | | | |
| | G, E, or none; X4 is I, | | | |
| | W, or none; X5 is G, D, | | | |
| | or none; X6 is or | | | |
| | none; X7 is N, L, or | | | |
| | none; X8 is N, Y, or | | | |
| | none; X9 is Y, or none; | | | |
| | X10 is D, E, or none; X11 | | | |
| | is G of none; and X12 is | | | |
| | M or none (SEQ ID NO: | | | |
| | 742) | | | |
| 743 | X1-X2-X3-X4-X5-X6-X7-X8- | Consensus_ | based on | CDR-H3 |
| | X9-X10-X11-X12-X13-X14-D, | 2 | Aho | |
| | wherein X1 is D or none; | | | |
| | X2 is R, G, or | | | |
| | none; X3 is G, E, W, or | | | |
| | none; X4 is A, | | | |
| | or none; X5 is G, | | | |
| | or none; X6 is F, L, | | | |
| | V, none; X7 is N, | | | |
| | L, G, D, or none; X8 is W | | | |
| | or Y; X9 is N, Y, or | | | |
| | none; X10 is Y, A, or | | | |
| | none; X11 is D, E, N, or | | | |
| | none; X12 is G or none; | | | |
| | X13 is F or Y; and X14 is | | | |
| | M or none (SEQ ID NO: | | | |
| | 743) | | | |
| 736 | X1-X2-X3-X4-X5-X6-X7-W- | Consensus_ | based on | CDR-H3 |
| | X8-X9-X10-X11-F-X12-D- | 1 | Cross- | |
| | X13, wherein X1 is D or | | linking | |
| | none; X2 is R, K, or G; | | | |
| | X3 is G, E, or none; X4 | | | |
| | is I, W, or none; X5 is | | | |
| | G, D, or none; X6 is F, | | | |
| | L, or none; X7 is N, L, | | | |
| | or none; X8 is N, Y, or | | | |
| | none; X9 is Y, or none; | | | |
| | X10 is D, E, or none; X11 | | | |
| | is G or none; X12 is M or | | | |
| | none; and X13 is Y or V | | | |
| | (SEQ ID NO: 736) | | | |
| 737 | X1-X2-X3-X4-X5-X6-X7-X8- | Consensus_ | based on | CDR-H3 |
| | X9-X10-X11-X12-X13-X14-D- | 2 | Cross- | |
| | X15, wherein X1 is D or | | linking | |
| | none; X2 is R, K, G, P, | | | |
| | or none; X3 is G, E, W, | | | |
| | or none; X4 is I, W, A, | | | |
| | N, or none; X5 is G, D, | | | |
| | Y, or none; X6 is F, L, | | | |
| | P, H, V, or none; X7 is | | | |
| | N, L, G, D, or none; X8 | | | |
| | is W or Y; X9 is N, Y, or | | | |
| | none; X10 is Y, A, or | | | |
| | none; X11 is D, E, N, or | | | |
| | none; X12 is G or none; | | | |
| | X13 is F or Y; X14 is M | | | |
| | or none; and X15 is Y or | | | |
| | V (SEQ ID NO: 737) | | | |
| 744 | X4, wherein X1 is R of S; | Consensus_ | based on | CDR-L1 |
| | X2 is N or S; X3 is N, H | 1 | Kabat | |
| | or W; and X4 is G or A | | | |
| | (SEQ ID NO: 744) | | | |
| 745 | R-A-S-Q-X1-I-X2-X3-X4-L- | Consensus_ | based on | CDR-L1 |
| | X5, wherein X1 is G, S, | 2 | Kabat | |
| | or D; X2 is R, S, or N; | | | |
| | X3 is N or S; X4 is N, H, | | | |
| | W, R, or Y; and X5 is G, | | | |
| | A, ox N (SEQ ID NO: 745) | | | |
| 744 | R-A-S-Q-G-I-X1-X2-X3-1- | Consensus_ | based on | CDR-L1 |
| | X4, wherein X1 is R or S; | 1 | Chothia | |
| | X2 is N or S; X3 is N, H | | | |
| | or W; and X4 is G or A | | | |
| | (SEQ ID NO: 744) | | | |
| 745 | R-A-S-Q-X1-I-X2-X3-X4-L- | Consensus_ | based on | CDR-L1 |
| | X5, wherein X1 is G, S, | 2 | Chothia | |
| | or D; X2 is R, S, or N; | | | |
| | X3 is N or S; X4 is N, H, | | | |
| | W, R, or Y; and X5 is G, | | | |
| | A, or N (SEQ ID NO: 745) | | | |
| 744 | R-A-S-Q-G-I-X1-X2-X3-1- | Consensus_ | based on | CDR-L1 |
| | X4, wherein X1 is R or S; | 1 | AbM | |
| | X2 is N or S; X3 is N, H | | | |
| | or W; and X4 is G or A | | | |
| | (SEQ ID NO: 744) | | | |
| 745 | R-A-S-Q-X1-I-X2-X3-X4-L- | Consensus_ | based on | CDR-L1 |
| | X5, wherein X1 is G, S, | 2 | AbM | |
| | or D; X2 is R, S, or N; | | | |
| | X3 is N or S; X4 is N, H, | | | |
| | W, R, ox Y; and X5 is G, | | | |
| | A, or N (SEQ ID NO: 745) | | | |
| 746 | X1-X2-X3-L-X4-W-X5, | Consensus_ | based on | CDR-L1 |
| | wherein X1 is R or S; X2 | 1 | Contact | |
| | is N or S; X3 is N, H or | | | |
| | W; X4 is G or A; and X5 | | | |
| | is Y or F (SEQ ID NO: | | | |
| | 746) | | | |
| 747 | X1-X2-X3-I-X4-W-X5, | Consensus_ | based on | CDR-L1 |
| | wherein X1 is R, S, of N; | 2 | Contact | |
| | X2 is N or S; X3 is N, H, | | | |
| | W, R, or Y; X4 is G, A, | | | |
| | of N; and X5 Y or E (SEQ | | | |
| | ID NO: 747) | | | |
| 748 | Q-G-I-X1-X2-X3, wherein | Consensus_ | based on | CDR-L1 |
| | X1 is R or S; X2 is N or | 1 | IMGT | |
| | S; and X3 is N, H or W | | | |
| | (SEQ ID NO: 748) | | | |
| 749 | Q-X1-I-X2-X3-X4, wherein | Consensus_ | based on | CDR-L1 |
| | X1 is G, S, or D; X2 is | 2 | IMGT | |
| | R, S, or N; X3 is N or S; | | | |
| | and X4 is N, H, W, R, or | | | |
| | Y (SEQ ID NO: 749) | | | |
| 750 | A-S-Q-G-I-X1-X2-X3, | Consensus_ | based on | CDR-L1 |
| | wherein X1 is R or S; X2 | 1 | Aho | |
| | is N or S; and X3 is N, H | | | |
| | or W (SEQ ID NO: 750) | | | |
| 751 | A-S-Q-X1-I-X2-X3-X4, | Consensus_ | based on | CDR-L1 |
| | wherein X1 is G, S, or D; | 2 | Aho | |
| | X2 is R, S, or N; X3 is N | | | |
| | or S; and X4 is N, H, W, | | | |
| | R, or Y (SEQ ID NO: 751) | | | |
| 744 | R-A-S-Q-G-I-X1-X2-X3-I- | Consensus_ | based on | CDR-L1 |
| | X4, wherein X1 is R or S; | 1 | Cross- | |
| | X2 is N or S; X3 is N, H | | linking | |
| | or W; and X4 is G or A | | | |
| | (SEQ ID NO: 744) | | | |
| 745 | R-A-S-Q-X1-I-X2-X3-X4-L- | Consensus_ | based on | CDR-L1 |
| | X5, wherein X1 is G, S, | 2 | Cross- | |
| | or D; X2 is R, S, or N; | | linking | |
| | X3 is N or S; X4 is N, H, | | | |
| | W, R, or Y; and X5 is G, | | | |
| | A, or N (SEQ ID NO: 745) | | | |
| 752 | A-X1-S-S-I-Q-S, wherein | Consensus_ | based on | CDR-L2 |
| | X1 is A or E (SEQ ID NO: | 1 | Kabat | |
| | 752) | | | |
| 753 | X1-X2-S-X3-L-X4-S, | Consensus_ | based on | CDR-L2 |
| | wherein X1 is A, K, or S; | 2 | Kabat | |
| | X2 is A, E, or V; X3 is | | | |
| | S, N, or T; and X4 is Q | | | |
| | or E (SEQ ID NO: 753) | | | |
| 752 | A-X1-S-S-I-Q-S, wherein | Consensus_ | based on | CDR-L2 |
| | X1 is A or E (SEQ ID NO: | 1 | Chothia | |
| | 752) | | | |
| 753 | X1-X2-S-X3-L-X4-S, | Consensus_ | based on | CDR-L2 |
| | wherein X1 is A, K, or S; | 2 | Chothia | |
| | X2 is A, E, or V; X3 is | | | |
| | S, N, or T; and X4 is Q | | | |
| | or E (SEQ ID NO: 753) | | | |
| 752 | A-X1-S-S-I-Q-S, wherein | Consensus_ | based on | CDR-L2 |
| | X1 is A or E (SEQ ID NO: | 1 | AbM | |
| | 752) | | | |
| 753 | X1-X2-S-X3-L-X4-S, | Consensus_ | based on | CDR-12 |
| | wherein X1 is A, K, or S; | 2 | AbM | |
| | X2 is A, E, or V; X3 is | | | |
| | S, N, or T; and X4 is Q | | | |
| | or E (SEQ ID NO: 753) | | | |
| 754 | X1-L-I-Y-A-X2-S-S-I-Q, | Consensus_ | based on | CDR-L2 |
| | wherein X1 is L or S; and | 1 | Contact | |
| | X2 is A or E (SEQ ID NO: | | | |
| | 754) | | | |
| 755 | X1-L-X2-Y-X3-X4-S-X5-L- | Consensus_ | based on | CDR-L2 |
| | X6, wherein X1 is L or S; | 2 | Contact | |
| | X2 is I or V; X3 is A, K, | | | |
| | or S; X4 is A, E, or V; | | | |
| | X5 is S, N, or T; and X6 | | | |
| | is Q or E (SEQ ID NO: | | | |
| | 755) | | | |
| 756 | A-X1, wherein X1 is A or | Consensus_ | based on | CDR-L2 |
| | E (SEQ ID NO: 756) | 1 | IMGT | |
| 757 | X1-X2, wherein X1 is A, | Consensus_ | based on | CDR-L2 |
| | K, or S; and X2 is A, E, | 2 | IMGT | |
| | or V (SEQ ID NO: 757) | | | |
| 758 | wherein X1 is A or E (SEQ | Consensus_ | based on | CDR-L2 |
| | ID NO: 758) | 1 | Aho | |
| 759 | X1-X2-S-X3-L-X4-S-G-V-P- | Consensus_ | based on | CDR-12 |
| | S-R, wherein X1 is A, K, | 2 | Aho | |
| | or S; X2 is A, E, or V; | | | |
| | X3 is S, N, or T; and X4 | | | |
| | is Q or E (SEQ ID NO: | | | |
| | 759) | | | |
| 752 | A-X1-S-S-I-Q-S, wherein | Consensus_ | based on | CDR-L2 |
| | X1 is A or E (SEQ ID NO: | 1 | Cross- | |
| | 752) | | linking | |
| 753 | X1-X2-S-X3-L-X4-S, | Consensus_ | based on | CDR-L2 |
| | wherein X1 is A, K, or S; | 2 | Cross- | |
| | X2 is A, E, or V; X3 is | | linking | |
| | S, N, or T; and X4 is Q | | | |
| | ox E (SEQ ID NO: 753) | | | |
| 760 | X1-Q-X2-X3-X4-X5-P-X6-T, | Consensus_ | based on | CDR-L3 |
| | wherein X1 is L or Q; X2 | 1 | Kabat | |
| | is D, Y, or A; X3 is F, | | | |
| | V, or N; X4 is N, T, or | | | |
| | S; X5 is Y or F; X6 is Y | | | |
| | or L (SEQ ID NO: 760) | | | |
| 761 | X1-Q-X2-X3-X4-X5-X6-X7-T, | Consensus_ | based on | CDR-L3 |
| | wherein X1 is L or Q; X2 | 2 | Kabat | |
| | is D, Y, A, or S; X3 is | | | |
| | F, V, N, or Y; X4 is N, | | | |
| | T, or S; X5 is Y, F, T, | | | |
| | or V; X6 is P or S; X7 is | | | |
| | Y, L or R (SEQ ID NO: | | | |
| | 761) | | | |
| 760 | X1-Q-X2-X3-X4-X5-P-X6-T; | Consensus_ | based on | CDR-L3 |
| | wherein X1 is L or Q: X2 | 1 | Chothia | |
| | is D, Y, or A; X3 is F, | | | |
| | V, or N; X4 is N, T, or | | | |
| | S; X5 is Y or F; X6 is Y | | | |
| | or L (SEQ ID NO: 760) | | | |
| 761 | X1-Q-X2-X3-X4-X5-X6-X7-T, | Consensus_ | based on | CDR-L3 |
| | wherein X1 is L or Q; X2 | 2 | Chothia | |
| | is D, Y, A, or S; X3 is | | | |
| | F, V, N, or Y; X4 is N, | | | |
| | T, or S; X5 is Y, F, T | | | |
| | or V; X6 is P or S; X7 is | | | |
| | Y, L or R (SEQ ID NO: | | | |
| | 761) | | | |
| 760 | X1-Q-X2-X3-X4-X5-P-X6-T, | Consensus_ | based on | CDR-L3 |
| | wherein X1 is L or Q; X2 | 1 | AbM | |
| | is D, Y, or A; X3 is E, | | | |
| | V, or N; X4 is N, T, or | | | |
| | S; X5 is Y or F; X6 is Y | | | |
| | or L (SEQ ID NO: 760) | | | |
| 761 | X1-Q-X2-X3-X4-X5-X6-X7-T, | Consensus_ | based on | CDR-L3 |
| | wherein X1 is L or Q; X2 | 2 | AbM | |
| | is D, Y, A, or S; X3 is | | | |
| | E, V, N, or Y; X4 is N, | | | |
| | T, or S; X5 is Y, F, T; | | | |
| | or V; X6 is P or S; X7 is | | | |
| | Y, L or R (SEQ ID NO: | | | |
| | 761) | | | |
| 762 | wherein X1 is L or Q; X2 | Consensus_ | based on | CDR-L3 |
| | is D, Y, or A; X3 is E, | 1 | Contact | |
| | V, or N: X4 is N, T, or | | | |
| | S; X5 is Y or F; X6 is Y | | | |
| | or L (SEQ ID NO: 762) | | | |
| 763 | X1-Q-X2-X3-X4-X5-X6-X7, | Consensus_ | based on | CDR-L3 |
| | wherein X1 is L or Q; X2 | 2 | Contact | |
| | is D, Y, A, or S; X3 is | | | |
| | F, V, N, or Y; X4 is N, | | | |
| | T, or S; X5 is Y, F, T; | | | |
| | or V; X6 is P or S; X7 is | | | |
| | Y, L or R (SEQ ID NO: | | | |
| | 763) | | | |
| 760 | X1-Q-X2-X3-X4-X5-P-X6-T, | Consensus_ | based on | CDR-L3 |
| | wherein X1 is L or Q; X2 | 1 | IMGT | |
| | is D, Y, or A; X3 is F, | | | |
| | V, or N; X4 is N, T, or | | | |
| | S; X5 is Y or F; X6 is Y | | | |
| | or L (SEQ ID NO: 760) | | | |
| 761 | X1-Q-X2-X3-X4-X5-X6-X7-T, | Consensus_ | based on | CDR-L3 |
| | wherein X1 is L or Q; X2 | 2 | IMGT | |
| | is D, Y, A, or S; X3 is | | | |
| | F, V, N, or Y; X4 is N, | | | |
| | T, or S; X5 is Y, F, T, | | | |
| | or V; X6 is P or S; X7 is | | | |
| | Y, L or R (SEQ ID NO: | | | |
| | 761) | | | |
| 764 | X1-X2-X3-X4-P-X5, wherein | Consensus_ | based on | CDR-L3 |
| | X1 is D, Y, or A; X2 is | 1 | Aho | |
| | F, V, or N; X3 is N, T, | | | |
| | or S; X4 is Y or F; X5 is | | | |
| | Y or L (SEQ ID NO: 764) | | | |
| 765 | X1-X2-X3-X4-X5-X6, | Consensus_ | based on | CDR-L3 |
| | wherein X1 is D, Y, A, or | 2 | Aho | |
| | S; X2 is F, V, N, or Y; | | | |
| | X3 is N, T, or S; X4 is | | | |
| | Y, F, T, or V; X5 is P or | | | |
| | S; X6 is Y, L or R (SEQ | | | |
| | ID NO: 765) | | | |
| 760 | X1-Q-X2-X3-X4-X5-P-X6-T, | Consensus_ | based on | CDR-L3 |
| | wherein X1 is L or Q; X2 | 1 | Cross- | |
| | is D, Y, or A; X3 is F, | | linking | |
| | V, or N; X4 is N, T, or | | | |
| | S; X5 is Y ox F; X6 is Y | | | |
| | or L (SEQ ID NO: 760) | | | |
| 761 | X1-Q-X2-X3-X4-X5-X6-X7-T, | Consensus_ | based on | CDR-L3 |
| | wherein X1 is L or Q; X2 | 2 | Cross- | |
| | is D, Y, A, or S; X3 is | | linking | |
| | F, V, N, or Y; X4 is N, | | | |
| | T, or S; X5 is Y, F, T, | | | |
| | or V; X6 is P or S; X7 is | | | |
| | Y, L or R (SEQ ID NO: | | | |
| | 761) | | | |
| 500 | ASTKGPSVFPLAPSSKSTSGGTAAL | CONSTANT_ | | |
| | GCLVKDYFPEPVTVSWNSGALTSGV | L234A_L235A | | |
| | HTFPAVLQSSGLYSLSSVVTVPSSS | | | |
| | LGTQTYICNVNHKPSNTKVDKKVEP | | | |
| | KSCDKTHTCPPCPAPEAAGGPSVFL | | | |
| | FPPKPKDTLMISRTPEVTCVVVDVS | | | |
| | HEDPEVKFNWYVDGVEVHNAKTKPR | | | |
| | EEQYNSTYRVVSVLTVLHQDWLNGK | | | |
| | EYKCKVSNKALPAPIEKTISKAKGQ | | | |
| | PREPQVYTLPPSRDELTKNQVSLTC | | | |
| | LVKGFYPSDIAVEWESNGQPENNYK | | | |
| | TTPPVLDSDGSFFLYSKLTVDKSRW | | | |
| | QQGNVFSCSVMHEALHNHYTQKSLS | | | |
| | LSPGK | | | |
| 501 | ASTKGPSVFPLAPSSKSTSGGTAAL | CONSTANT_ | | |
| | GCLVKDYFPEPVTVSWNSGALTSGV | L235E | | |
| | HTFPAVLQSSGLYSLSSVVTVPSSS | | | |
| | LGTQTYICNVNHKPSNTKVDKKVEP | | | |
| | KSCDKTHTCPPCPAPELEGGPSVFL | | | |
| | FPPKPKDTLMISRTPEVTCVVVDVS | | | |
| | HEDPEVKFNWYVDGVEVHNAKTKPR | | | |
| | EEQYNSTYRVVSVLTVLHQDWLNGK | | | |
| | EYKCKVSNKALPAPIEKTISKAKGQ | | | |
| | PREPQVYTLPPSRDELTKNQVSLTC | | | |
| | LVKGFYPSDIAVEWESNGQPENNYK | | | |
| | TTPPVLDSDGSFFLYSKLTVDKSRW | | | |
| | QQGNVFSCSVMHEALHNHYTQKSLS | | | |
| | LSPGK | | | |
| 502 | ASTKGPSVFPLAPSSKSTSGGTAAL | CONSTANT_ | | |
| | GCLVKDYFPEPVTVSWNSGALTSGV | L234A_ | | |
| | HTFPAVLQSSGLYSLSSVVTVPSSS | L235A_ | | |
| | LGTQTYICNVNHKPSNTKVDKKVEP | G237A | | |
| | KSCDKTHTCPPCPAPEAAGAPSVFL | | | |
| | FPPKPKDTLMISRTPEVTCVVVDVS | | | |
| | HEDPEVKFNWYVDGVEVHNAKTKPR | | | |
| | EEQYNSTYRVVSVLTVLHQDWLNGK | | | |
| | EYKCKVSNKALPAPIEKTISKAKGQ | | | |
| | PREPQVYTLPPSRDELTKNQVSLTC | | | |
| | LVKGFYPSDIAVEWESNGQPENNYK | | | |
| | TTPPVLDSDGSFFLYSKLTVDKSRW | | | |
| | QQGNVFSCSVMHEALHNHYTQKSLS | | | |
| | LSPGK | | | |
| 503 | ASTKGPSVFPLAPSSKSTSGGTAAL | CONSTANT_ | | |
| | GCLVKDYFPEPVTVSWNSGALTSGV | L234A_ | | |
| | HTFPAVLQSSGLYSLSSVVTVPSSS | L235E_ | | |
| | LGTQTYICNVNHKPSNTKVDKKVEP | G237A | | |
| | KSCDKTHTCPPCPAPEAEGAPSVFL | | | |
| | FPPKPKDTLMISRTPEVTCVVVDVS | | | |
| | HEDPEVKFNWYVDGVEVHNAKTKPR | | | |
| | EEQYNSTYRVVSVLTVLHQDWLNGK | | | |
| | EYKCKVSNKALPAPIEKTISKAKGQ | | | |
| | PREPQVYTLPPSRDELTKNQVSLTC | | | |
| | LVKGFYPSDIAVEWESNGQPENNYK | | | |
| | TTPPVLDSDGSFFLYSKLTVDKSRW | | | |
| | QQGNVFSCSVMHEALHNHYTQKSLS | | | |
| | LSPGK | | | |
| 504 | ASTKGPSVFPLAPSSKSTSGGTAAL | CONSTANT_ | | |
| | GCLVKDYFPEPVTVSWNSGALTSGV | L234A_ | | |
| | HTFPAVLQSSGLYSLSSVVTVPSSS | L235A_ | | |
| | LGTQTYICNVNHKPSNTKVDKKVEP | P329A | | |
| | KSCDKTHTCPPCPAPEAAGGPSVFL | | | |
| | FPPKPKDTLMISRTPEVTCVVVDVS | | | |
| | HEDPEVKFNWYVDGVEVHNAKTKPR | | | |
| | EEQYNSTYRVVSVLTVLHQDWLNGK | | | |
| | EYKCKVSNKALAAPIEKTISKAKGQ | | | |
| | PREPQVYTLPPSRDELTKNQVSLTC | | | |
| | LVKGFYPSDIAVEWESNGQPENNYK | | | |
| | TTPPVLDSDGSFFLYSKLTVDKSRW | | | |
| | QQGNVFSCSVMHEALHNHYTQKSLS | | | |
| | LSPGK | | | |
| 505 | ASTKGPSVFPLAPSSKSTSGGTAAL | CONSTANT_ | | |
| | GCLVKDYFPEPVTVSWNSGALTSGV | L234A_ | | |
| | HTFPAVLQSSGLYSLSSVVTVPSSS | L235A_ | | |
| | LGTQTYICNVNHKPSNTKVDKKVEP | P329G | | |
| | KSCDKTHTCPPCPAPEAAGGPSVFL | | | |
| | FPPKPKDTLMISRTPEVTCVVVDVS | | | |
| | HEDPEVKFNWYVDGVEVHNAKTKPR | | | |
| | EEQYNSTYRVVSVLTVLHQDWLNGK | | | |
| | EYKCKVSNKALAAPIEKTISKAKGQ | | | |
| | PREPQVYTLPPSRDELTKNQVSLTC | | | |
| | LVKGFYPSDIAVEWESNGQPENNYK | | | |
| | TTPPVLDSDGSFFLYSKLTVDKSRW | | | |
| | QQGNVFSCSVMHEALHNHYTQKSLS | | | |
| | LSPGK | | | |
| 506 | ASTKGPSVFPLAPSSKSTSGGTAAL | CONSTANT_ | | |
| | GCLVKDYFPEPVTVSWNSGALTSGV | P329A | | |
| | HTFPAVLQSSGLYSLSSVVTVPSSS | | | |
| | LGTQTYICNVNHKPSNTKVDKKVEP | | | |
| | KSCDKTHTCPPCPAPEAAGGPSVFL | | | |
| | FPPKPKDTLMISRTPEVTCVVVDVS | | | |
| | HEDPEVKFNWYVDGVEVHNAKTKPR | | | |
| | EEQYNSTYRVVSVLTVLHQDWLNGK | | | |
| | EYKCKVSNKALAAPIEKTISKAKGQ | | | |
| | PREPQVYTLPPSRDELTKNQVSLTC | | | |
| | LVKGFYPSDIAVEWESNGQPENNYK | | | |
| | TTPPVLDSDGSFFLYSKLTVDKSRW | | | |
| | QQGNVFSCSVMHEALHNHYTQKSLS | | | |
| | LSPGK | | | |
| 507 | ASTKGPSVFPLAPSSKSTSGGTAAL | CONSTANT_ | | |
| | GCLVKDYFPEPVTVSWNSGALTSGV | L234E_ | | |
| | HTFPAVLQSSGLYSLSSVVTVPSSS | L235F_ | | |
| | LGTQTYICNVNHKPSNTKVDKKVEP | P331S | | |
| | KSCDKTHTCPPCPAPEEFGGPSVFL | | | |
| | FPPKPKDTLMISRTPEVTCVVVDVS | | | |
| | HEDPEVKFNWYVDGVEVHNAKTKPR | | | |
| | EEQYNSTYRVVSVLTVLHQDWLNGK | | | |
| | EYKCKVSNKALPASIEKTISKAKGQ | | | |
| | PREPQVYTLPPSRDELTKNQVSLTC | | | |
| | LVKGFYPSDIAVEWESNGQPENNYK | | | |
| | TTPPVLDSDGSFFLYSKLTVDKSRW | | | |
| | QQGNVFSCSVMHEALHNHYTQKSLS | | | |
| | LSPGK | | | |
| 508 | ASTKGPSVFPLAPSSKSTSGGTAAL | CONSTANT_ | | |
| | GCLVKDYFPEPVTVSWNSGALTSGV | D265A_N297G | | |
| | HTFPAVLQSSGLYSLSSVVTVPSSS | | | |
| | LGTQTYICNVNHKPSNTKVDKKVEP | | | |
| | KSCDKTHTCPPCPAPELLGGPSVFL | | | |
| | FPPKPKDTLMISRTPEVTCVVVAVS | | | |
| | HEDPEVKFNWYVDGVEVHNAKTKPR | | | |
| | EEQYGSTYRVVSVLTVLHQDWLNGK | | | |
| | EYKCKVSNKALPAPIEKTISKAKGQ | | | |
| | PREPQVYTLPPSRDELTKNQVSLTC | | | |
| | LVKGFYPSDIAVEWESNGQPENNYK | | | |
| | TTPPVLDSDGSFFLYSKLTVDKSRW | | | |
| | QQGNVFSCSVMHEALHNHYTQKSLS | | | |
| | LSPGK | | | |
| 509 | ASTKGPSVFPLAPSSKSTSGGTAAL | CONSTANT_ | | |
| | GCLVKDYFPEPVTVSWNSGALTSGV | N297G | | |
| | HTFPAVLQSSGLYSLSSVVTVPSSS | | | |
| | LGTQTYICNVNHKPSNTKVDKKVEP | | | |
| | KSCDKTHTCPPCPAPELLGGPSVFL | | | |
| | FPPKPKDTLMISRTPEVTCVVVDVS | | | |
| | HEDPEVKFNWYVDGVEVHNAKTKPR | | | |
| | EEQYGSTYRVVSVLTVLHQDWLNGK | | | |
| | EYKCKVSNKALPAPIEKTISKAKGQ | | | |
| | PREPQVYTLPPSRDELTKNQVSLTC | | | |
| | LVKGFYPSDIAVEWESNGQPENNYK | | | |
| | TTPPVLDSDGSFFLYSKLTVDKSRW | | | |
| | QQGNVFSCSVMHEALHNHYTQKSLS | | | |
| | LSPGK | | | |
| 510 | ASTKGPSVFPLAPCSRSTSESTAAL | CONSTANT_ | | |
| | GCLVKDYFPEPVTVSWNSGALTSGV | S228P | | |
| | HTFPAVLQSSGLYSLSSVVTVPSSS | | | |
| | LGTKTYTCNVDHKPSNTKVDKRVES | | | |
| | KYGPPCPPCPAPEFLGGPSVFLFPP | | | |
| | KPKDTLMISRTPEVTCVVVDVSQED | | | |
| | PEVQFNWYVDGVEVHNAKTKPREEQ | | | |
| | FNSTYRVVSVLTVLHQDWLNGKEYK | | | |
| | CKVSNKGLPSSIEKTISKAKGQPRE | | | |
| | PQVYTLPPSQEEMTKNQVSLTCLVK | | | |
| | GFYPSDIAVEWESNGQPENNYKTTP | | | |
| | PVLDSDGSFFLYSRLTVDKSRWQEG | | | |
| | NVFSCSVMHEALHNHYTQKSLSLSL | | | |
| | GK | | | |
| 511 | ASTKGPSVEPLAPCSRSTSESTAAL | CONSTANT_ | | |
| | GCLVKDYFPEPVTVSWNSGALTSGV | S228P_L235E | | |
| | HTFPAVLQSSGLYSLSSVVTVPSSS | | | |
| | LGTKTYTCNVDHKPSNTKVDKRVES | | | |
| | KYGPPCPPCPAPEFEGGPSVFLFPP | | | |
| | KPKDTLMISRTPEVTCVVVDVSQED | | | |
| | PEVQFNWYVDGVEVHNAKTKPREEQ | | | |
| | FNSTYRVVSVLTVLHQDWLNGKEYK | | | |
| | CKVSNKGLPSSIEKTISKAKGQPRE | | | |
| | PQVYTLPPSQEEMTKNQVSLTCLVK | | | |
| | GFYPSDIAVEWESNGQPENNYKTTP | | | |
| | PVLDSDGSFFLYSRLTVDKSRWQEG | | | |
| | NVFSCSVMHEALHNHYTQKSLSLSL | | | |
| | GK | | | |
| 512 | ASTKGPSVFPLAPCSRSTSESTAAL | CONSTANT_ | | |
| | GCLVKDYFPEPVTVSWNSGALTSGV | S228P_ | | |
| | HTFPAVLQSSGLYSLSSVVTVPSSS | F234A | | |
| | LGTKTYTCNVDHKPSNTKVDKRVES | L235A | | |
| | KYGPPCPPCPAPEAAGGPSVFLFPP | | | |
| | KPKDTLMISRTPEVTCVVVDVSQED | | | |
| | PEVQFNWYVDGVEVHNAKTKPREEQ | | | |
| | FNSTYRVVSVLTVLHQDWLNGKEYK | | | |
| | CKVSNKGLPSSIEKTISKAKGQPRE | | | |
| | PQVYTLPPSQEEMTKNQVSLTCLVK | | | |
| | GFYPSDIAVEWESNGQPENNYKTTP | | | |
| | PVLDSDGSFFLYSRLTVDKSRWQEG | | | |
| | NVFSCSVMHEALHNHYTQKSLSLSL | | | |
| | GK | | | |
| 774 | GYTFTDY | CLONE_111 | Chothia | CDR-H1 |
| 775 | GYTFTDYAMH | | AbM | |
| 776 | DYAMH | | Kabat | |
| 777 | TDYAMH | | Contact | |
| 778 | GYTFTDYA | | IMGT | |
| 779 | ASGYTFTDYA | | Aho | |
| 780 | IPLIDT | CLONE_111 | Chothia | CDR-H2 |
| 781 | SIIPLIDTTS | | AbM | |
| 782 | SIIPLIDTTSYNQKFKG | | Kabat | |
| 783 | WIGSIIPLIDTTS | | Contact | |
| 784 | IIPLIDTT | | IMGT | |
| 785 | IIPLIDTTSYNQKFKGK | | Aho | |
| 786 | LGSRYLEGFDY | CLONE_111 | Chothia | CDR-H3 |
| 787 | LGSRYLEGFDY | | AbM | |
| 788 | LGSRYLEGFDY | | Kabat | |
| 789 | ARLGSRYLEGFD | | Contact | |
| 790 | ARLGSRYLEGFDY | | IMGT | |
| 791 | LGSRYLEGFD | | Aho | |
| 792 | LASQDIGNYLS | CLONE_111 | Chothia | CDR-L1 |
| 793 | LASQDIGNYLS | | AbM | |
| 794 | LASQDIGNYLS | | Kabat | |
| 795 | GNYLSWY | | Contact | |
| 796 | QDIGNY | | IMGT | |
| 797 | ASQDIGNY | | Aho | |
| 798 | GATNLED | CLONE_111 | Chothia | CDR-L2 |
| 799 | GATNLED | | AbM | |
| 800 | GATNLED | | Kabat | |
| 801 | LMIYGATNLE | | Contact | |
| 802 | GA | | IMGT | |
| 803 | GATNLEDGVPSR | | Aho | |
| 804 | HQYYEYPLT | CLONE_111 | Chothia | CDR-L3 |
| 805 | HQYYEYPLT | | AbM | |
| 806 | HQYYEYPLT | | Kabat | |
| 807 | HQYYEYPL | | Contact | |
| 808 | HQYYEYPLT | | IMGT | |
| 809 | YYEYPL | | Aho | |

1. A pharmaceutical composition comprising an antibody or antigen binding fragment thereof that binds CD30L (anti-CD30L antibody or antigen binding fragment), wherein the antibody or antigen binding fragment thereof comprises:
(a) a CDR-H1 comprising the amino acid sequence set forth in any one of SEQ ID NOs: 100-139, 220-234, 465-489, 628-641, and 712-723;
(b) a CDR-H2 comprising the amino acid sequence set forth in any one of SEQ ID NOs: 140-179, 235-249, 490-499, 513-527, 642-655, and 724-735;
(c) a CDR-H3 comprising the amino acid sequence set forth in any one of SEQ Id NOs: 180-219, 250-264, 528-552, 656-669, and 736-743;
(d) a CDR-L1 comprising the amino acid sequence set forth in any one of SEQ ID NOs: 300-339, 420-434, 553-577, 670-683, and 744-751;
(d) CDR-L2 comprising the amino acid sequence set forth in any one of SEQ ID NOs: 340-379, 435-449, 578-602, 684-697, and 752-759; and/or
(e) a CDR-L3 comprising the amino acid sequence set forth in any one of SEQ ID NOs: 380-419, 450-464, 603-627, 698-711, and 760-765.

2. The pharmaceutical composition of claim 1, wherein the antibody or antigen binding fragment thereof comprises:
(a) a CDR-H1 comprising the amino acid sequence set forth in any one of SEQ ID NOs: 100-109, 628, 635, and 712-723;
(b) a CDR-H2 comprising the amino acid sequence set forth in any one of SEQ ID NOs: 140-149, 642, 649, and 724-735;
(c) a CDR-H3 comprising the amino acid sequence set forth in any one of SEQ ID NOs: 180-189, 656, 663, and 736-743;
(d) a CDR-L1 comprising the amino acid sequence set forth in any one of SEQ ID NOs: 300-309, 670, 677, and 744-751;
(e) a CDR-L2 comprising the amino acid sequence set forth in any one of SEQ ID NOs: 340-349, 684, 691, and 752-759; and/or
(f) a CDR-L3 comprising the amino acid sequence set forth in any one of SEQ ID Nos: 380-389, 698, 705, and 760-765.

3. The pharmaceutical composition of claim 1 or 2, wherein the antibody or antigen binding fragment thereof comprises:
(a) a CDR-H1 comprising the amino acid sequence set forth in any one of SEQ ID NOs: 100-104;
(b) a CDR-H2 comprising the amino acid sequence set forth in any one of SEQ ID NOs: 140-144;
(c) a CDR-H3 comprising the amino acid sequence set forth in any one of SEQ ID NOs: 180-184;
(d) a CDR-L1 comprising the amino acid sequence set forth in any one of SEQ ID NOs: 300-304;
(e) a CDR-L2 comprising the amino acid sequence set forth in any one of SEQ ID NOs: 340-344; and/or
(f) a CDR-L3 comprising the amino acid sequence set forth in any one of SEQ ID NOs: 380-384.

4. The pharmaceutical composition of claim 1 or 2, wherein the antibody or antigen binding fragment thereof comprises:
(a) a CDR-H1 comprising the amino acid sequence set forth in any one of SEQ ID NOs: 105-109, 628, 635, and 712-723;
(b) a CDR-H2 comprising the amino acid sequence set forth in any one of SEQ ID NOs: 145-149, 642, 649, and 724-735;
(c) a CDR-H3 comprising the amino acid sequence set forth in any one of SEQ ID NOs: 185-189, 656, 663, and 736-743;
(d) a CDR-L1 comprising the amino acid sequence set forth in any one of SEQ ID NOs: 305-309, 670, 677, and 744-751;
(e) a CDR-L2 comprising the amino acid sequence set forth in any one of SEQ ID NOs: 345-349, 684, 691, and 752-759; and/or
(f) a CDR-L3 comprising the amino acid sequence set forth in any one of SEQ ID NOs: 385-389, 698, 705, and 760-765.

5. The pharmaceutical composition of any one of claims 1 to 4, wherein the antibody or antigen binding fragment thereof comprises an immunoglobulin variable region heavy chain and an immunoglobulin variable region light chain, wherein:
(a) the immunoglobulin variable region heavy chain comprises an amino acid sequence having at least about 90, 95, 97, 98, 99, or 100% sequence identity to any one of SEQ ID NOs: 1 and 2; and/or
(b) the immunoglobulin variable region light chain comprises an amino acid sequence having at least about 90, 95, 97, 98, 99, or 100% sequence identity to any one of SEQ ID NOs: 3 and 4.

6. A pharmaceutical composition comprising an antibody or antigen binding fragment thereof that binds CD30L (anti-CD30L antibody or antigen binding fragment), wherein the antibody or antigen binding fragment thereof binds to an epitope comprising one or more amino acids in CD30L selected from the group consisting of N165, K166, I168, K169, and D234, wherein the amino acids are numbered according to the amino acid sequence of CD30L as set forth in SEQ ID NO:33.

7. The pharmaceutical composition of claim 6, wherein the antibody or antigen binding fragment thereof binds to an epitope comprising:
(i) any one amino acid in CD30L selected from the group consisting of N165, K166, I168, K169, and D234;
(ii) any two amino acids in CD30L selected from the group consisting of N165, K166, I168, K169, and D234;
(iii) any three amino acids in CD30L selected from the group consisting of N165, K166, I168, K169, and D234;
(iv) any four amino acids in CD30L selected from the group consisting of N165, K166, I168, K169, and D234; or
(v) any five amino acids in CD30L selected from the group consisting of N165, K166, I168, K169, and D234;
wherein the amino acids are numbered according to the amino acid sequence of CD30L as set forth in SEQ ID NO:33.

8. A pharmaceutical composition comprising an antibody or antigen binding fragment thereof that binds CD30L (anti-CD30L antibody or antigen binding fragment), wherein the antibody or antigen binding fragment thereof binds to an epitope comprising one or more amino acids in CD30L selected from the group consisting of H167, S217, and D118, wherein the amino acids are numbered according to the amino acid sequence of CD30L as set forth in SEQ ID NO:33.

9. The pharmaceutical composition of claim 6 or 7, wherein the antibody or antigen binding fragment thereof binds to an epitope comprising:
(i) any one amino acid in CD30L selected from the group consisting of H167, S217, and D118;
(ii) any two amino acids in CD30L selected from the group consisting of H167, S217, and D118; or
(iii) any three amino acids in CD30L selected from the group consisting of H167, S217, and D118;
wherein the amino acids are numbered according to the amino acid sequence of CD30L as set forth in SEQ ID NO:33.

10. A pharmaceutical composition comprising an antibody or antigen binding fragment thereof that binds CD30L (anti-CD30L antibody or antigen binding fragment), wherein the antibody or antigen binding fragment thereof binds to an epitope comprising one or more amino acids in CD30L selected from the group consisting of D118, N165, K166, H167, I168, K169, S217, and D234, wherein the amino acids are numbered according to the amino acid sequence of CD30L as set forth in SEQ ID NO:33.

11. The pharmaceutical composition of claim 10, wherein the antibody or antigen binding fragment thereof binds to an epitope comprising:
(i) any one amino acid in CD30L selected from the group consisting of D118, N165, K166, H167, I168, K169, S217, and D234;
(ii) any two amino acids in CD30L selected from the group consisting of D118, N165, K166, H167, I168, K169, S217, and D234;
(iii) any three amino acids in CD30L selected from the group consisting of D118, N165, K166, H167, I168, K169, S217, and D234;
(iv) any four amino acids in CD30L selected from the group consisting of D118, N165, K166, H167, I168, K169, S217, and D234;
(v) any five amino acids in CD30L selected from the group consisting of D118, N165, K166, H167, I168, K169, S217, and D234;
(vi) any six amino acids in CD30L selected from the group consisting of D118, N165, K166, H167, I168, K169, S217, and D234;
(vii) any seven amino acids in CD30L selected from the group consisting of D118, N165, K166, H167, I168, K169, S217, and D234; or
(viii) any five amino acids in CD30L selected from the group consisting of D118, N165, K166, H167, I168, K169, S217, and D234;
wherein the amino acids are numbered according to the amino acid sequence of CD30L as set forth in SEQ ID NO:33.

12. A pharmaceutical composition comprising an anti-CD30L antibody or antigen binding fragment thereof, wherein the antibody or antigen binding fragment thereof binds to an epitope comprising K169 in CD30L, wherein the amino acids are numbered according to the amino acid sequence of CD30L as set forth in SEQ ID NO:33.

13. The pharmaceutical composition of claim 12, wherein the antibody or antigen binding fragment thereof binds to an epitope comprising D234 in CD30L, wherein the amino acids are numbered according to the amino acid sequence of CD30L as set forth in SEQ ID NO:33.

14. A pharmaceutical composition comprising an anti-CD30L antibody or antigen binding fragment thereof, wherein the antibody or antigen binding fragment thereof binds to an epitope comprising D234 in CD30L, wherein the amino acids are numbered according to the amino acid sequence of CD30L as set forth in SEQ ID NO:33

15. The pharmaceutical composition of claims 12 to 14, wherein the epitope further comprises N165 in CD30L, wherein the amino acids are numbered according to the amino acid sequence of CD30L as set forth in SEQ ID NO:33.

16. The pharmaceutical composition of any one of claims 12 to 15, wherein the epitope further comprises I168 in CD30L, wherein the amino acids are numbered according to the amino acid sequence of CD30L as set forth in SEQ ID NO:33.

17. The pharmaceutical composition of any one of claims 12 to 16, wherein the epitope further comprises K166 in CD30L, wherein the amino acids are numbered according to the amino acid sequence of CD30L as set forth in SEQ ID NO:33.

18. The pharmaceutical composition of any one of claims 12 to 17, wherein the epitope further comprises H167 in CD30L, wherein the amino acids are numbered according to the amino acid sequence of CD30L as set forth in SEQ ID NO:33.

19. The pharmaceutical composition of any one of claims 12 to 18, wherein the epitope further comprises S217 in CD30L, wherein the amino acids are numbered according to the amino acid sequence of CD30L as set forth in SEQ ID NO:33.

20. The pharmaceutical composition of any one of claims 12 to 19, wherein the epitope further comprises D118 in CD30L, wherein the amino acids are numbered according to the amino acid sequence of CD30L as set forth in SEQ ID NO:33.

21. The pharmaceutical composition of any one of claims 1 and 6 to 20, wherein the antibody or antigen binding fragment thereof comprises:
(a) a CDR-H1 comprising the amino acid sequence set forth in any one of SEQ ID NOs: 110-119, 629, 636, and 712-723;
(b) a CDR-H2 comprising the amino acid sequence set forth in any one of SEQ ID NOs: 150-159, 643, 650, and 724-735;
(c) a CDR-H3 comprising the amino acid sequence set forth in any one of SEQ ID NOs: 190-199, 657, 664, and 736-743;
(d) a CDR-L1 comprising the amino acid sequence set forth in any one of SEQ ID NOs: 310-319, 671, 678, and 744-751;
(e) a CDR-L2 comprising the amino acid sequence set forth in any one of SEQ ID NOs: 350-359, 685, 692, and 752-759; and/or
(f) a CDR-L3 comprising the amino acid sequence set forth in any one of SEQ ID NOs: 390-399, 699, 706, and 760-765.

22. The pharmaceutical composition of any one of claims 1 and 6 to 20, wherein the antibody or antigen binding fragment thereof comprises:
(a) a CDR-H1 comprising the amino acid sequence set forth in any one of SEQ ID NOs: 110-114;
(b) a CDR-H2 comprising the amino acid sequence set forth in any one of SEQ ID NOs: 150-154;
(c) a CDR-H3 comprising the amino acid sequence set forth in any one of SEQ ID NOs: 190-194;
(d) a CDR-L1 comprising the amino acid sequence set forth in any one of SEQ ID NOs: 310-314;
(e) a CDR-L2 comprising the amino acid sequence set forth in any one of SEQ ID NOs: 350-354; and/or
(f) a CDR-L3 comprising the amino acid sequence set forth in any one of SEQ ID NOs: 390-394.

23. The pharmaceutical composition of any one of claims 1 and 6 to 20, wherein the antibody or antigen binding fragment thereof comprises:
(a) a CDR-H1 comprising the amino acid sequence set forth in any one of SEQ ID NOs: 115-119, 629, 636, and 712-723;
(b) a CDR-H2 comprising the amino acid sequence set forth in any one of SEQ ID NOs: 155-159, 643, 650, and 724-735;
(c) a CDR-H3 comprising the amino acid sequence set forth in any one of SEQ ID NOs: 195-199, 657, 664, and 736-743;
(d) a CDR-L1 comprising the amino acid sequence set forth in any one of SEQ ID NOs: 315-319, 671, 678, and 744-751;
(e) a CDR-L2 comprising the amino acid sequence set forth in any one of SEQ ID NOs: 355-359, 685, 692, and 752-759; and/or
(f) a CDR-L3 comprising the amino acid sequence set forth in any one of SEQ ID NOs: 395-399, 699, 706, and 760-765.

24. The pharmaceutical composition of any one of claims 1, 6 to 20, and 21 to 23, wherein the antibody or antigen binding fragment thereof comprises an immunoglobulin variable region heavy chain and an immunoglobulin variable region light chain, wherein:
(a) the immunoglobulin variable region heavy chain comprises an amino acid sequence having at least about 90, 95, 97, 98, 99, or 100% sequence identity to any one of SEQ ID NOs: 5 and 6; and/or
(b) the immunoglobulin variable region light chain comprises an amino acid sequence having at least about 90, 95, 97, 98, 99, or 100% sequence identity to any one of SEQ ID NOs: 7 and 8.

25. The pharmaceutical composition of claim 1, wherein the antibody or antigen binding fragment thereof comprises:
(a) a CDR-H1 comprising the amino acid sequence set forth in any one of SEQ ID NOs: 120-129, and 712-723;
(b) a CDR-H2 comprising the amino acid sequence set forth in any one of SEQ ID NOs: 160-169, and 724-735;
(c) a CDR-H3 comprising the amino acid sequence set forth in any one of SEQ ID NOs: 200-209, and 736-743;
(d) a CDR-L1 comprising the amino acid sequence set forth in any one of SEQ ID NOs: 320-329, and 744-751;
(e) a CDR-L2 comprising the amino acid sequence set forth in any one of SEQ ID NOs: 360-369, and 752-759; and/or
(f) a CDR-L3 comprising the amino acid sequence set forth in any one of SEQ ID NOs: 400-409, and 760-765.

26. The pharmaceutical composition of claim 1 or 25, wherein the antibody or antigen binding fragment thereof comprises:
(a) a CDR-H1 comprising the amino acid sequence set forth in any one of SEQ ID NOs: 120-124;
(b) a CDR-H2 comprising the amino acid sequence set forth in any one of SEQ ID NOs: 160-164;
(c) a CDR-H3 comprising the amino acid sequence set forth in any one of SEQ ID NOs: 200-204;
(d) a CDR-L1 comprising the amino acid sequence set forth in any one of SEQ ID NOs: 320-324;
(e) a CDR-L2 comprising the amino acid sequence set forth in any one of SEQ ID NOs: 360-364; and/or
(f) a CDR-L3 comprising the amino acid sequence set forth in any one of SEQ ID NOs: 400-404.

27. The pharmaceutical composition of claim 1 or 25, wherein the antibody or antigen binding fragment thereof comprises:
(a) a CDR-H1 comprising the amino acid sequence set forth in any one of SEQ ID NOs: 125-129, and 712-723;
(b) a CDR-H2 comprising the amino acid sequence set forth in any one of SEQ ID NOs: 165-169, and 724-735;
(c) a CDR-H3 comprising the amino acid sequence set forth in any one of SEQ ID NOs: 205-209, and 736-743;
(d) a CDR-L1 comprising the amino acid sequence set forth in any one of SEQ ID NOs: 325-329, and 744-751;
(e) a CDR-L2 comprising the amino acid sequence set forth in any one of SEQ ID NOs: 365-369, and 752-759; and/or
(f) a CDR-L3 comprising the amino acid sequence set forth in any one of SEQ ID NOs: 405-409, and 760-765.

28. The pharmaceutical composition of any one of claims 1 and 25 to 27, wherein the antibody or antigen binding fragment thereof comprises an immunoglobulin variable region heavy chain and an immunoglobulin variable region light chain, wherein:
(a) the immunoglobulin variable region heavy chain comprises an amino acid sequence having at least about 90, 95, 97, 98, 99, or 100% sequence identity to any one of SEQ ID NOs: 9 and 10; and/or
(b) the immunoglobulin variable region light chain comprises an amino acid sequence having at least about 90, 95, 97, 98, 99, or 100% sequence identity to any one of SEQ ID NOs: 11 and 12.

29. The pharmaceutical composition of claim 1, wherein the antibody or antigen binding fragment thereof comprises:
(a) a CDR-H1 comprising the amino acid sequence set forth in any one of SEQ ID NOs: 130-139, and 712-723;
(b) a CDR-H2 comprising the amino acid sequence set forth in any one of SEQ ID NOs: 170-179, and 724-735;
(c) a CDR-H3 comprising the amino acid sequence set forth in any one of SEQ ID NOs: 210-219, and 736-743;
(d) a CDR-L1 comprising the amino acid sequence set forth in any one of SEQ ID NOs: 330-339, and 744-751;
(e) a CDR-L2 comprising the amino acid sequence set forth in any one of SEQ ID NOs: 370-379, and 752-759; and/or
(f) a CDR-L3 comprising the amino acid sequence set forth in any one of SEQ ID NOs: 410-419, and 760-765.

30. The pharmaceutical composition of claim 1 or 29, wherein the antibody or antigen binding fragment thereof comprises:
(a) a CDR-H1 comprising the amino acid sequence set forth in any one of SEQ ID NOs: 130-134;
(b) a CDR-H2 comprising the amino acid sequence set forth in any one of SEQ ID NOs: 170-174;
(c) a CDR-H3 comprising the amino acid sequence set forth in any one of SEQ ID NOs: 210-214;
(d) a CDR-L1 comprising the amino acid sequence set forth in any one of SEQ ID NOs: 330-334;
(e) a CDR-L2 comprising the amino acid sequence set forth in any one of SEQ ID NOs: 370-374; and/or
(f) a CDR-L3 comprising the amino acid sequence set forth in any one of SEQ ID NOs: 410-414.

31. The pharmaceutical composition of claim 1 or 29, wherein the antibody or antigen binding fragment thereof comprises:
(a) a CDR-H1 comprising the amino acid sequence set forth in any one of SEQ ID NOs: 135-139, and 712-723;
(b) a CDR-H2 comprising the amino acid sequence set forth in any one of SEQ ID NOs: 175-179, and 724-735;
(c) a CDR-H3 comprising the amino acid sequence set forth in any one of SEQ ID NOs: 215-219, and 736-743;
(d) a CDR-L1 comprising the amino acid sequence set forth in any one of SEQ ID NOs: 335-339, and 744-751;
(e) a CDR-L2 comprising the amino acid sequence set forth in any one of SEQ ID NOs: 375-379, and 752-759; and/or
(f) a CDR-L3 comprising the amino acid sequence set forth in any one of SEQ ID NOs: 415-419, and 760-765.

32. The pharmaceutical composition of any one of claims 1 and 29 to 31, wherein the antibody or antigen binding fragment thereof comprises an immunoglobulin variable region heavy chain and an immunoglobulin variable region light chain, wherein:
(a) the immunoglobulin variable region heavy chain comprises an amino acid sequence having at least about 90, 95, 97, 98, 99, or 100% sequence identity to any one of SEQ ID NOs: 13 and 14; and/or
(b) the immunoglobulin variable region light chain comprises an amino acid sequence having at least about 90, 95, 97, 98, 99, or 100% sequence identity to any one of SEQ ID NOs: 15 and 16.

33. The pharmaceutical composition of claim 1, wherein the antibody or antigen binding fragment thereof comprises:
(a) a CDR-H1 comprising the amino acid sequence set forth in any one of SEQ ID NOs: 220-224, and 712-723;
(b) a CDR-H2 comprising the amino acid sequence set forth in any one of SEQ ID NOs: 235-239, and 724-735;
(c) a CDR-H3 comprising the amino acid sequence set forth in any one of SEQ ID NOs: 250-254, and 736-743;
(d) a CDR-L1 comprising the amino acid sequence set forth in any one of SEQ ID NOs: 420-424, and 744-751;
(e) a CDR-L2 comprising the amino acid sequence set forth in any one of SEQ ID NOs: 435-439, and 752-759; and/or
(f) a CDR-L3 comprising the amino acid sequence set forth in any one of SEQ ID NOs: 450-454, and 760-765.

34. The pharmaceutical composition of claim 1 or 33, wherein the antibody or antigen binding fragment thereof comprises an immunoglobulin variable region heavy chain and an immunoglobulin variable region light chain, wherein:
(a) the immunoglobulin variable region heavy chain comprises an amino acid sequence having at least about 90, 95, 97, 98, 99, or 100% sequence identity to SEQ ID NO: 17; and/or
(b) the immunoglobulin variable region light chain comprises an amino acid sequence having at least about 90, 95, 97, 98, 99, or 100% sequence identity to SEQ ID NO: 18.

35. The pharmaceutical composition of claim 1, wherein the antibody or antigen binding fragment thereof comprises:
(a) a CDR-H1 comprising the amino acid sequence set forth in any one of SEQ ID NOs: 225-229, and 712-723;
(b) a CDR-H2 comprising the amino acid sequence set forth in any one of SEQ ID NOs: 240-244, and 724-735;
(c) a CDR-H3 comprising the amino acid sequence set forth in any one of SEQ ID NOs: 255-259, and 736-743;
(d) a CDR-L1 comprising the amino acid sequence set forth in any one of SEQ ID NOs: 425-429, and 744-751;
(e) a CDR-L2 comprising the amino acid sequence set forth in any one of SEQ ID NOs: 440-444, and 752-759; and/or
(f) a CDR-L3 comprising the amino acid sequence set forth in any one of SEQ ID NOs: 455-459, and 760-765.

36. The pharmaceutical composition of claim 1 or 35, wherein the antibody or antigen binding fragment thereof comprises an immunoglobulin variable region heavy chain and an immunoglobulin variable region light chain, wherein:
(a) the immunoglobulin variable region heavy chain comprises an amino acid sequence having at least about 90, 95, 97, 98, 99, or 100% sequence identity to SEQ ID NO: 19; and/or
(b) the immunoglobulin variable region light chain comprises an amino acid sequence having at least about 90, 95, 97, 98, 99, or 100% sequence identity to SEQ ID NO: 20.

37. The pharmaceutical composition of claim 1, wherein the antibody or antigen binding fragment thereof comprises:
(a) a CDR-H1 comprising the amino acid sequence set forth in any one of SEQ ID NOs: 230-234, and 712-723;
(b) a CDR-H2 comprising the amino acid sequence set forth in any one of SEQ ID NOs: 245-249, and 724-735;
(c) a CDR-H3 comprising the amino acid sequence set forth in any one of SEQ ID NOs: 260-264, and 736-743;
(d) a CDR-L1 comprising the amino acid sequence set forth in any one of SEQ ID NOs: 430-434, and 744-751;
(e) a CDR-L2 comprising the amino acid sequence set forth in any one of SEQ ID NOs: 445-449, and 752-759; and/or
(f) a CDR-L3 comprising the amino acid sequence set forth in any one of SEQ ID NOs: 460-464, and 760-765.

38. The pharmaceutical composition of claim 1 or 37, wherein the antibody or antigen binding fragment thereof comprises an immunoglobulin variable region heavy chain and an immunoglobulin variable region light chain, wherein:
(a) the immunoglobulin variable region heavy chain comprises an amino acid sequence having at least about 90, 95, 97, 98, 99, or 100% sequence identity to SEQ ID NO: 21; and/or
(b) the immunoglobulin variable region light chain comprises an amino acid sequence having at least about 90, 95, 97, 98, 99, or 100% sequence identity to SEQ ID NO: 22.

39. The pharmaceutical composition of claim 1, wherein the antibody or antigen binding fragment thereof comprises:
(i)(a) a CDR-H1 comprising the amino acid sequence set forth in any one of SEQ ID NOs: 465-469, 631, 638, and 712-723; (b) a CDR-H2 comprising the amino acid sequence set forth in any one of SEQ ID NOs: 490-494, 645, 652, and 724-735; (c) a CDR-H3 comprising the amino acid sequence set forth in any one of SEQ ID NOs: 528-532, 659, 666, and 736-743; (d) a CDR-L1 comprising the amino acid sequence set forth in any one of SEQ ID NOs: 553-557, 673, 680, and 744-751; (e) a CDR-L2 comprising the amino acid sequence set forth in any one of SEQ ID NOs: 578-582, 687, 694, and 752-759; and/or (f) a CDR-L3 comprising the amino acid sequence set forth in any one of SEQ ID NOs: 603-607, 701, 708, and 760-765;
(ii)(a) a CDR-H1 comprising the amino acid sequence set forth in any one of SEQ ID NOs: 470-474, 632, 639, and 712-723; (b) a CDR-H2 comprising the amino acid sequence set forth in any one of SEQ ID NOs: 495-499, 646, 653, and 724-735; (c) a CDR-H3 comprising the amino acid sequence set forth in any one of SEQ ID NOs: 533-537, 660, 667, and 736-743; (d) a CDR-L1 comprising the amino acid sequence set forth in any one of SEQ ID NOs: 558-562, 674, 681, and 744-751; (e) a CDR-L2 comprising the amino acid sequence set forth in any one of SEQ ID NOs: 583-587, 688, 695, and 752-759; and/or (f) a CDR-L3 comprising the amino acid sequence set forth in any one of SEQ ID NOs: 608-612, 702, 709, and 760-765;
(iii)(a) a CDR-H1 comprising the amino acid sequence set forth in any one of SEQ ID NOs: 475-479, 633, 640, and 712-723; (b) a CDR-H2 comprising the amino acid sequence set forth in any one of SEQ ID NOs: 513-517, 647, 654, and 724-735; (c) a CDR-H3 comprising the amino acid sequence set forth in any one of SEQ ID NOs: 538-542, 661, 668, and 736-743; (d) a CDR-L1 comprising the amino acid sequence set forth in any one of SEQ ID NOs: 563-567, 675, 682, and 744-751; (e) a CDR-L2 comprising the amino acid sequence set forth in any one of SEQ ID NOs: 588-592, 689, 696, and 752-759; and/or (f) a CDR-L3 comprising the amino acid sequence set forth in any one of SEQ ID NOs: 613-617, 703, 710, and 760-765;
(iv)(a) a CDR-H1 comprising the amino acid sequence set forth in any one of SEQ ID NOs: 480-484, 630, 637, and 712-723; (b) a CDR-H2 comprising the amino acid sequence set forth in any one of SEQ ID NOs: 518-522, 644, 651, and 724-735; (c) a CDR-H3 comprising the amino acid sequence set forth in any one of SEQ ID NOs: 543-547, 658, 665, and 736-743; (d) a CDR-L1 comprising the amino acid sequence set forth in any one of SEQ ID NOs: 568-572, 672, 679, and 744-751; (e) a CDR-L2 comprising the amino acid sequence set forth in any one of SEQ ID NOs: 593-597, 686, 693, and 752-759; and/or (f) a CDR-L3 comprising the amino acid sequence set forth in any one of SEQ ID NOs: 618-622, 700, 707, and 760-765; or
(v)(a) a CDR-H1 comprising the amino acid sequence set forth in any one of SEQ ID NOs: 485-489, 634, 641, and 712-723; (b) a CDR-H2 comprising the amino acid sequence set forth in any one of SEQ ID NOs: 523-527, 648, 655, and 724-735; (c) a CDR-H3 comprising the amino acid sequence set forth in any one of SEQ ID NOs: 548-552, 662, 669, and 736-743; (d) a CDR-L1 comprising the amino acid sequence set forth in any one of SEQ ID NOs: 573-577, 676, 683, and 744-751; (e) a CDR-L2 comprising the amino acid sequence set forth in any one of SEQ ID NOs: 598-602, 690, 697, and 752-759; and/or (f) a CDR-L3 comprising the amino acid sequence set forth in any one of SEQ ID NOs: 623-627, 704, 711, and 760-765.

40. The pharmaceutical composition of any one of claims 1 and 6 to 20, wherein the antibody or antigen binding fragment thereof comprises:
(i)(a) a CDR-H1 comprising the amino acid sequence set forth in any one of SEQ ID NOs: 712-723; (b) a CDR-H2 comprising the amino acid sequence set forth in any one of SEQ ID NOs: 724-735; (c) a CDR-H3 comprising the amino acid sequence set forth in any one of SEQ ID NOs: 736-743; (d) a CDR-L1 comprising the amino acid sequence set forth in any one of SEQ ID NOs: 744-751; (e) a CDR-L2 comprising the amino acid sequence set forth in any one of SEQ ID NOs: 752-759; and/or (f) a CDR-L3 comprising the amino acid sequence set forth in any one of SEQ ID NOs: 760-765;
(ii)(a) a CDR-H1 comprising the amino acid sequence set forth in any one of SEQ ID NOs: 712, 714, 716, 718, 720, and 722; (b) a CDR-H2 comprising the amino acid sequence set forth in any one of SEQ ID NOs: 724, 726, 728, 730, 732, and 734; (c) a CDR-H3 comprising the amino acid sequence set forth in any one of SEQ ID NOs: 736, 738, 740, and 742; (d) a CDR-L1 comprising the amino acid sequence set forth in any one of SEQ ID NOs: 744, 746, 748, and 750; (e) a CDR-L2 comprising the amino acid sequence set forth in any one of SEQ ID NOs: 752, 754, 756, and 758; and/or (f) a CDR-L3 comprising the amino acid sequence set forth in any one of SEQ ID NOs: 760, 762, and 764;
(iii)(a) a CDR-H1 comprising the amino acid sequence set forth in any one of SEQ ID NOs: 713, 715, 717, 719, 721, and 723; (b) a CDR-H2 comprising the amino acid sequence set forth in any one of SEQ ID NOs: 725, 727, 729, 731, 733, and 735; (c) a CDR-H3 comprising the amino acid sequence set forth in any one of SEQ ID NOs: 737, 739, 741, and 743; (d) a CDR-L1 comprising the amino acid sequence set forth in any one of SEQ ID NOs: 745, 747, 749, and 751; (e) a CDR-L2 comprising the amino acid sequence set forth in any one of SEQ ID NOs: 753, 755, 757, and 759; and/or (f) a CDR-L3 comprising the amino acid sequence set forth in any one of SEQ ID NOs: 761, 763, and 765;
(iv)(a) a CDR-H1 comprising the amino acid sequence set forth in SEQ ID NO: 712; (b) a CDR-H2 comprising the amino acid sequence set forth in SEQ ID NO: 730; (c) a CDR-H3 comprising the amino acid sequence set forth in SEQ ID NO: 736; (d) a CDR-L1 comprising the amino acid sequence set forth in SEQ ID NO: 744; (e) a CDR-L2 comprising the amino acid sequence set forth SEQ ID NO: 752; and/or (f) a CDR-L3 comprising the amino acid sequence set forth SEQ ID NO: 760;
(v)(a) a CDR-H1 comprising the amino acid sequence set forth in SEQ ID NO: 713; (b) a CDR-H2 comprising the amino acid sequence set forth in SEQ ID NO: 731; (c) a CDR-H3 comprising the amino acid sequence set forth in SEQ ID NO: 737; (d) a CDR-L1 comprising the amino acid sequence set forth in SEQ ID NO: 745; (e) a CDR-L2 comprising the amino acid sequence set forth SEQ ID NO: 753; and/or (f) a CDR-L3 comprising the amino acid sequence set forth SEQ ID NO: 761;
(vi)(a) a CDR-H1 comprising the amino acid sequence set forth in SEQ ID NO: 712; (b) a CDR-H2 comprising the amino acid sequence set forth in SEQ ID NO: 724; (c) a CDR-H3 comprising the amino acid sequence set forth in SEQ ID NO: 736; (d) a CDR-L1 comprising the amino acid sequence set forth in SEQ ID NO: 744; (e) a CDR-L2 comprising the amino acid sequence set forth SEQ ID NO: 752; and/or (f) a CDR-L3 comprising the amino acid sequence set forth SEQ ID NO: 760;
(vii)(a) a CDR-H1 comprising the amino acid sequence set forth in SEQ ID NO: 713; (b) a CDR-H2 comprising the amino acid sequence set forth in SEQ ID NO: 725; (c) a CDR-H3 comprising the amino acid sequence set forth in SEQ ID NO: 737; (d) a CDR-L1 comprising the amino acid sequence set forth in SEQ ID NO: 745; (e) a CDR-L2 comprising the amino acid sequence set forth SEQ ID NO: 753; and/or (f) a CDR-L3 comprising the amino acid sequence set forth SEQ ID NO: 761;
(viii)(a) a CDR-H1 comprising the amino acid sequence set forth in SEQ ID NO: 714; (b) a CDR-H2 comprising the amino acid sequence set forth in SEQ ID NO: 726; (c) a CDR-H3 comprising the amino acid sequence set forth in SEQ ID NO: 736; (d) a CDR-L1 comprising the amino acid sequence set forth in SEQ ID NO: 744; (e) a CDR-L2 comprising the amino acid sequence set forth SEQ ID NO: 752; and/or (f) a CDR-L3 comprising the amino acid sequence set forth SEQ ID NO: 760;
(ix)(a) a CDR-H1 comprising the amino acid sequence set forth in SEQ ID NO: 715; (b) a CDR-H2 comprising the amino acid sequence set forth in SEQ ID NO: 727; (c) a CDR-H3 comprising the amino acid sequence set forth in SEQ ID NO: 737; (d) a CDR-L1 comprising the amino acid sequence set forth in SEQ ID NO: 745; (e) a CDR-L2 comprising the amino acid sequence set forth SEQ ID NO: 753; and/or (f) a CDR-L3 comprising the amino acid sequence set forth SEQ ID NO: 761;
(x)(a) a CDR-H1 comprising the amino acid sequence set forth in SEQ ID NO: 716; (b) a CDR-H2 comprising the amino acid sequence set forth in SEQ ID NO: 728; (c) a CDR-H3 comprising the amino acid sequence set forth in SEQ ID NO: 736; (d) a CDR-L1 comprising the amino acid sequence set forth in SEQ ID NO: 744; (e) a CDR-L2 comprising the amino acid sequence set forth SEQ ID NO: 752; and/or (f) a CDR-L3 comprising the amino acid sequence set forth SEQ ID NO: 760;
(xi)(a) a CDR-H1 comprising the amino acid sequence set forth in SEQ ID NO: 717; (b) a CDR-H2 comprising the amino acid sequence set forth in SEQ ID NO: 729; (c) a CDR-H3 comprising the amino acid sequence set forth in SEQ ID NO: 737; (d) a CDR-L1 comprising the amino acid sequence set forth in SEQ ID NO: 745; (e) a CDR-L2 comprising the amino acid sequence set forth SEQ ID NO: 753; and/or (f) a CDR-L3 comprising the amino acid sequence set forth SEQ ID NO: 761;
(xii)(a) a CDR-H1 comprising the amino acid sequence set forth in SEQ ID NO: 718; (b) a CDR-H2 comprising the amino acid sequence set forth in SEQ ID NO: 730; (c) a CDR-H3 comprising the amino acid sequence set forth in SEQ ID NO: 738; (d) a CDR-L1 comprising the amino acid sequence set forth in SEQ ID NO: 746; (e) a CDR-L2 comprising the amino acid sequence set forth SEQ ID NO: 754; and/or (f) a CDR-L3 comprising the amino acid sequence set forth SEQ ID NO: 762;
(xiii)(a) a CDR-H1 comprising the amino acid sequence set forth in SEQ ID NO: 719; (b) a CDR-H2 comprising the amino acid sequence set forth in SEQ ID NO: 731; (c) a CDR-H3 comprising the amino acid sequence set forth in SEQ ID NO: 739; (d) a CDR-L1 comprising the amino acid sequence set forth in SEQ ID NO: 747; (e) a CDR-L2 comprising the amino acid sequence set forth SEQ ID NO: 755; and/or (f) a CDR-L3 comprising the amino acid sequence set forth SEQ ID NO: 763;
(xiv)(a) a CDR-H1 comprising the amino acid sequence set forth in SEQ ID NO: 720; (b) a CDR-H2 comprising the amino acid sequence set forth in SEQ ID NO: 732; (c) a CDR-H3 comprising the amino acid sequence set forth in SEQ ID NO: 740; (d) a CDR-L1 comprising the amino acid sequence set forth in SEQ ID NO: 748; (e) a CDR-L2 comprising the amino acid sequence set forth SEQ ID NO: 756; and/or (f) a CDR-L3 comprising the amino acid sequence set forth SEQ ID NO: 760;
(xv)(a) a CDR-H1 comprising the amino acid sequence set forth in SEQ ID NO: 721; (b) a CDR-H2 comprising the amino acid sequence set forth in SEQ ID NO: 733; (c) a CDR-H3 comprising the amino acid sequence set forth in SEQ ID NO: 741; (d) a CDR-L1 comprising the amino acid sequence set forth in SEQ ID NO: 749; (e) a CDR-L2 comprising the amino acid sequence set forth SEQ ID NO: 757; and/or (f) a CDR-L3 comprising the amino acid sequence set forth SEQ ID NO: 761;
(xvi)(a) a CDR-H1 comprising the amino acid sequence set forth in SEQ ID NO: 722; (b) a CDR-H2 comprising the amino acid sequence set forth in SEQ ID NO: 734; (c) a CDR-H3 comprising the amino acid sequence set forth in SEQ ID NO: 742; (d) a CDR-L1 comprising the amino acid sequence set forth in SEQ ID NO: 750; (e) a CDR-L2 comprising the amino acid sequence set forth SEQ ID NO: 758; and/or (f) a CDR-L3 comprising the amino acid sequence set forth SEQ ID NO: 764; or
(xvii)(a) a CDR-H1 comprising the amino acid sequence set forth in SEQ ID NO: 723; (b) a CDR-H2 comprising the amino acid sequence set forth in SEQ ID NO: 735; (c) a CDR-H3 comprising the amino acid sequence set forth in SEQ ID NO: 743; (d) a CDR-L1 comprising the amino acid sequence set forth in SEQ ID NO: 751; (e) a CDR-L2 comprising the amino acid sequence set forth SEQ ID NO: 759; and/or (f) a CDR-L3 comprising the amino acid sequence set forth SEQ ID NO: 765.

41. The pharmaceutical composition of any one of claims 1 to 4 and 40, wherein the antibody or antigen binding fragment thereof comprises:
(i)(a) a CDR-H1 comprising the amino acid sequence set forth in SEQ ID NO: 635; (b) a CDR-H2 comprising the amino acid sequence set forth in SEQ ID NO: 649; (c) a CDR-H3 comprising the amino acid sequence set forth in SEQ ID NO: 663; (d) a CDR-L1 comprising the amino acid sequence set forth in SEQ ID NO: 677; (e) a CDR-L2 comprising the amino acid sequence set forth SEQ ID NO: 691; and/or (f) a CDR-L3 comprising the amino acid sequence set forth SEQ ID NO: 705;
(ii)(a) a CDR-H1 comprising the amino acid sequence set forth in SEQ ID NO: 107; (b) a CDR-H2 comprising the amino acid sequence set forth in SEQ ID NO: 147; (c) a CDR-H3 comprising the amino acid sequence set forth in SEQ ID NO: 187; (d) a CDR-L1 comprising the amino acid sequence set forth in SEQ ID NO: 307; (e) a CDR-L2 comprising the amino acid sequence set forth SEQ ID NO: 347; and/or (f) a CDR-L3 comprising the amino acid sequence set forth SEQ ID NO: 387;
(iii)(a) a CDR-H1 comprising the amino acid sequence set forth in SEQ ID NO: 105; (b) a CDR-H2 comprising the amino acid sequence set forth in SEQ ID NO: 145; (c) a CDR-H3 comprising the amino acid sequence set forth in SEQ ID NO: 185; (d) a CDR-L1 comprising the amino acid sequence set forth in SEQ ID NO: 305; (e) a CDR-L2 comprising the amino acid sequence set forth SEQ ID NO: 345; and/or (f) a CDR-L3 comprising the amino acid sequence set forth SEQ ID NO: 385;
(iv)(a) a CDR-H1 comprising the amino acid sequence set forth in SEQ ID NO: 106; (b) a CDR-H2 comprising the amino acid sequence set forth in SEQ ID NO: 146; (c) a CDR-H3 comprising the amino acid sequence set forth in SEQ ID NO: 186; (d) a CDR-L1 comprising the amino acid sequence set forth in SEQ ID NO: 306; (e) a CDR-L2 comprising the amino acid sequence set forth SEQ ID NO: 346; and/or (f) a CDR-L3 comprising the amino acid sequence set forth SEQ ID NO: 386;
(v)(a) a CDR-H1 comprising the amino acid sequence set forth in SEQ ID NO: 108; (b) a CDR-H2 comprising the amino acid sequence set forth in SEQ ID NO: 148; (c) a CDR-H3 comprising the amino acid sequence set forth in SEQ ID NO: 188; (d) a CDR-L1 comprising the amino acid sequence set forth in SEQ ID NO: 308; (e) a CDR-L2 comprising the amino acid sequence set forth SEQ ID NO: 348; and/or (f) a CDR-L3 comprising the amino acid sequence set forth SEQ ID NO: 388;
(vi)(a) a CDR-H1 comprising the amino acid sequence set forth in SEQ ID NO: 109; (b) a CDR-H2 comprising the amino acid sequence set forth in SEQ ID NO: 149; (c) a CDR-H3 comprising the amino acid sequence set forth in SEQ ID NO: 189; (d) a CDR-L1 comprising the amino acid sequence set forth in SEQ ID NO: 309; (e) a CDR-L2 comprising the amino acid sequence set forth SEQ ID NO: 349; and/or (f) a CDR-L3 comprising the amino acid sequence set forth SEQ ID NO: 389; or
(vii)(a) a CDR-H1 comprising the amino acid sequence set forth in SEQ ID NO: 628; (b) a CDR-H2 comprising the amino acid sequence set forth in SEQ ID NO: 642; (c) a CDR-H3 comprising the amino acid sequence set forth in SEQ ID NO: 656; (d) a CDR-L1 comprising the amino acid sequence set forth in SEQ ID NO: 670; (e) a CDR-L2 comprising the amino acid sequence set forth SEQ ID NO: 684; and/or (f) a CDR-L3 comprising the amino acid sequence set forth SEQ ID NO: 698.

42. The pharmaceutical composition of any one of claims 1, 6 to 23, and 40, wherein the antibody or antigen binding fragment thereof comprises:
(i)(a) a CDR-H1 comprising the amino acid sequence set forth in SEQ ID NO: 636; (b) a CDR-H2 comprising the amino acid sequence set forth in SEQ ID NO: 650; (c) a CDR-H3 comprising the amino acid sequence set forth in SEQ ID NO: 664; (d) a CDR-L1 comprising the amino acid sequence set forth in SEQ ID NO: 678; (e) a CDR-L2 comprising the amino acid sequence set forth SEQ ID NO: 692; and/or (f) a CDR-L3 comprising the amino acid sequence set forth SEQ ID NO: 706;
(ii)(a) a CDR-H1 comprising the amino acid sequence set forth in SEQ ID NO: 117; (b) a CDR-H2 comprising the amino acid sequence set forth in SEQ ID NO: 157; (c) a CDR-H3 comprising the amino acid sequence set forth in SEQ ID NO: 197; (d) a CDR-L1 comprising the amino acid sequence set forth in SEQ ID NO: 317; (e) a CDR-L2 comprising the amino acid sequence set forth SEQ ID NO: 357; and/or (f) a CDR-L3 comprising the amino acid sequence set forth SEQ ID NO: 397;
(iii)(a) a CDR-H1 comprising the amino acid sequence set forth in SEQ ID NO: 115; (b) a CDR-H2 comprising the amino acid sequence set forth in SEQ ID NO: 155; (c) a CDR-H3 comprising the amino acid sequence set forth in SEQ ID NO: 195; (d) a CDR-L1 comprising the amino acid sequence set forth in SEQ ID NO: 315; (e) a CDR-L2 comprising the amino acid sequence set forth SEQ ID NO: 355; and/or (f) a CDR-L3 comprising the amino acid sequence set forth SEQ ID NO: 395;
(iv)(a) a CDR-H1 comprising the amino acid sequence set forth in SEQ ID NO: 116; (b) a CDR-H2 comprising the amino acid sequence set forth in SEQ ID NO: 156; (c) a CDR-H3 comprising the amino acid sequence set forth in SEQ ID NO: 196; (d) a CDR-L1 comprising the amino acid sequence set forth in SEQ ID NO: 316; (e) a CDR-L2 comprising the amino acid sequence set forth SEQ ID NO: 356; and/or (f) a CDR-L3 comprising the amino acid sequence set forth SEQ ID NO: 396;
(v)(a) a CDR-H1 comprising the amino acid sequence set forth in SEQ ID NO: 118; (b) a CDR-H2 comprising the amino acid sequence set forth in SEQ ID NO: 158; (c) a CDR-H3 comprising the amino acid sequence set forth in SEQ ID NO: 198; (d) a CDR-L1 comprising the amino acid sequence set forth in SEQ ID NO: 318; (e) a CDR-L2 comprising the amino acid sequence set forth SEQ ID NO: 358; and/or (f) a CDR-L3 comprising the amino acid sequence set forth SEQ ID NO: 398;
(vi)(a) a CDR-H1 comprising the amino acid sequence set forth in SEQ ID NO: 119; (b) a CDR-H2 comprising the amino acid sequence set forth in SEQ ID NO: 159; (c) a CDR-H3 comprising the amino acid sequence set forth in SEQ ID NO: 199; (d) a CDR-L1 comprising the amino acid sequence set forth in SEQ ID NO: 319; (e) a CDR-L2 comprising the amino acid sequence set forth SEQ ID NO: 359; and/or (f) a CDR-L3 comprising the amino acid sequence set forth SEQ ID NO: 399; or
(vii)(a) a CDR-H1 comprising the amino acid sequence set forth in SEQ ID NO: 629; (b) a CDR-H2 comprising the amino acid sequence set forth in SEQ ID NO: 643; (c) a CDR-H3 comprising the amino acid sequence set forth in SEQ ID NO: 657; (d) a CDR-L1 comprising the amino acid sequence set forth in SEQ ID NO: 671; (e) a CDR-L2 comprising the amino acid sequence set forth SEQ ID NO: 685; and/or (f) a CDR-L3 comprising the amino acid sequence set forth SEQ ID NO: 699.

43. The pharmaceutical composition of claim 1 or 40, wherein the antibody or antigen binding fragment thereof comprises:
(i)(a) a CDR-H1 comprising the amino acid sequence set forth in SEQ ID NO: 637; (b) a CDR-H2 comprising the amino acid sequence set forth in SEQ ID NO: 651; (c) a CDR-H3 comprising the amino acid sequence set forth in SEQ ID NO: 665; (d) a CDR-L1 comprising the amino acid sequence set forth in SEQ ID NO: 679; (e) a CDR-L2 comprising the amino acid sequence set forth SEQ ID NO: 693; and/or (f) a CDR-L3 comprising the amino acid sequence set forth SEQ ID NO: 707;
(ii)(a) a CDR-H1 comprising the amino acid sequence set forth in SEQ ID NO: 482; (b) a CDR-H2 comprising the amino acid sequence set forth in SEQ ID NO: 520; (c) a CDR-H3 comprising the amino acid sequence set forth in SEQ ID NO: 545; (d) a CDR-L1 comprising the amino acid sequence set forth in SEQ ID NO: 570; (e) a CDR-L2 comprising the amino acid sequence set forth SEQ ID NO: 595; and/or (f) a CDR-L3 comprising the amino acid sequence set forth SEQ ID NO: 620;
(iii)(a) a CDR-H1 comprising the amino acid sequence set forth in SEQ ID NO: 480; (b) a CDR-H2 comprising the amino acid sequence set forth in SEQ ID NO: 518; (c) a CDR-H3 comprising the amino acid sequence set forth in SEQ ID NO: 543; (d) a CDR-L1 comprising the amino acid sequence set forth in SEQ ID NO: 568; (e) a CDR-L2 comprising the amino acid sequence set forth SEQ ID NO: 593; and/or (f) a CDR-L3 comprising the amino acid sequence set forth SEQ ID NO: 618;
(iv)(a) a CDR-H1 comprising the amino acid sequence set forth in SEQ ID NO: 481; (b) a CDR-H2 comprising the amino acid sequence set forth in SEQ ID NO: 519; (c) a CDR-H3 comprising the amino acid sequence set forth in SEQ ID NO: 544; (d) a CDR-L1 comprising the amino acid sequence set forth in SEQ ID NO: 569; (e) a CDR-L2 comprising the amino acid sequence set forth SEQ ID NO: 594; and/or (f) a CDR-L3 comprising the amino acid sequence set forth SEQ ID NO: 619;
(v)(a) a CDR-H1 comprising the amino acid sequence set forth in SEQ ID NO: 483; (b) a CDR-H2 comprising the amino acid sequence set forth in SEQ ID NO: 521; (c) a CDR-H3 comprising the amino acid sequence set forth in SEQ ID NO: 546; (d) a CDR-L1 comprising the amino acid sequence set forth in SEQ ID NO: 571; (e) a CDR-L2 comprising the amino acid sequence set forth SEQ ID NO: 596; and/or (f) a CDR-L3 comprising the amino acid sequence set forth SEQ ID NO: 621;
(vi)(a) a CDR-H1 comprising the amino acid sequence set forth in SEQ ID NO: 484; (b) a CDR-H2 comprising the amino acid sequence set forth in SEQ ID NO: 522; (c) a CDR-H3 comprising the amino acid sequence set forth in SEQ ID NO: 547; (d) a CDR-L1 comprising the amino acid sequence set forth in SEQ ID NO: 572; (e) a CDR-L2 comprising the amino acid sequence set forth SEQ ID NO: 597; and/or (f) a CDR-L3 comprising the amino acid sequence set forth SEQ ID NO: 622; or
(vii)(a) a CDR-H1 comprising the amino acid sequence set forth in SEQ ID NO: 630; (b) a CDR-H2 comprising the amino acid sequence set forth in SEQ ID NO: 644; (c) a CDR-H3 comprising the amino acid sequence set forth in SEQ ID NO: 658; (d) a CDR-L1 comprising the amino acid sequence set forth in SEQ ID NO: 672; (e) a CDR-L2 comprising the amino acid sequence set forth SEQ ID NO: 686; and/or (f) a CDR-L3 comprising the amino acid sequence set forth SEQ ID NO: 700.

44. The pharmaceutical composition of claim 1 or 40, wherein the antibody or antigen binding fragment thereof comprises:
(i)(a) a CDR-H1 comprising the amino acid sequence set forth in SEQ ID NO: 638; (b) a CDR-H2 comprising the amino acid sequence set forth in SEQ ID NO: 652; (c) a CDR-H3 comprising the amino acid sequence set forth in SEQ ID NO: 666; (d) a CDR-L1 comprising the amino acid sequence set forth in SEQ ID NO: 680; (e) a CDR-L2 comprising the amino acid sequence set forth SEQ ID NO: 694; and/or (f) a CDR-L3 comprising the amino acid sequence set forth SEQ ID NO: 708;
(ii)(a) a CDR-H1 comprising the amino acid sequence set forth in SEQ ID NO: 467; (b) a CDR-H2 comprising the amino acid sequence set forth in SEQ ID NO: 492; (c) a CDR-H3 comprising the amino acid sequence set forth in SEQ ID NO: 530; (d) a CDR-L1 comprising the amino acid sequence set forth in SEQ ID NO: 555; (e) a CDR-L2 comprising the amino acid sequence set forth SEQ ID NO: 580; and/or (f) a CDR-L3 comprising the amino acid sequence set forth SEQ ID NO: 605;
(iii)(a) a CDR-H1 comprising the amino acid sequence set forth in SEQ ID NO: 465; (b) a CDR-H2 comprising the amino acid sequence set forth in SEQ ID NO: 490; (c) a CDR-H3 comprising the amino acid sequence set forth in SEQ ID NO: 528; (d) a CDR-L1 comprising the amino acid sequence set forth in SEQ ID NO: 553; (e) a CDR-L2 comprising the amino acid sequence set forth SEQ ID NO: 578; and/or (f) a CDR-L3 comprising the amino acid sequence set forth SEQ ID NO: 603;
(iv)(a) a CDR-H1 comprising the amino acid sequence set forth in SEQ ID NO: 466; (b) a CDR-H2 comprising the amino acid sequence set forth in SEQ ID NO: 491; (c) a CDR-H3 comprising the amino acid sequence set forth in SEQ ID NO: 529; (d) a CDR-L1 comprising the amino acid sequence set forth in SEQ ID NO: 554; (e) a CDR-L2 comprising the amino acid sequence set forth SEQ ID NO: 579; and/or (f) a CDR-L3 comprising the amino acid sequence set forth SEQ ID NO: 604;
(v)(a) a CDR-H1 comprising the amino acid sequence set forth in SEQ ID NO: 468; (b) a CDR-H2 comprising the amino acid sequence set forth in SEQ ID NO: 493; (c) a CDR-H3 comprising the amino acid sequence set forth in SEQ ID NO: 531; (d) a CDR-L1 comprising the amino acid sequence set forth in SEQ ID NO: 556; (e) a CDR-L2 comprising the amino acid sequence set forth SEQ ID NO: 581; and/or (f) a CDR-L3 comprising the amino acid sequence set forth SEQ ID NO: 606;
(vi)(a) a CDR-H1 comprising the amino acid sequence set forth in SEQ ID NO: 469; (b) a CDR-H2 comprising the amino acid sequence set forth in SEQ ID NO: 494; (c) a CDR-H3 comprising the amino acid sequence set forth in SEQ ID NO: 532; (d) a CDR-L1 comprising the amino acid sequence set forth in SEQ ID NO: 557; (e) a CDR-L2 comprising the amino acid sequence set forth SEQ ID NO: 582; and/or (f) a CDR-L3 comprising the amino acid sequence set forth SEQ ID NO: 607; or
(vii)(a) a CDR-H1 comprising the amino acid sequence set forth in SEQ ID NO: 631; (b) a CDR-H2 comprising the amino acid sequence set forth in SEQ ID NO: 645; (c) a CDR-H3 comprising the amino acid sequence set forth in SEQ ID NO: 659; (d) a CDR-L1 comprising the amino acid sequence set forth in SEQ ID NO: 673; (e) a CDR-L2 comprising the amino acid sequence set forth SEQ ID NO: 687; and/or (f) a CDR-L3 comprising the amino acid sequence set forth SEQ ID NO: 701.

45. The pharmaceutical composition of claim 1 or 40, wherein the antibody or antigen binding fragment thereof comprises:
(i)(a) a CDR-H1 comprising the amino acid sequence set forth in SEQ ID NO: 639; (b) a CDR-H2 comprising the amino acid sequence set forth in SEQ ID NO: 653; (c) a CDR-H3 comprising the amino acid sequence set forth in SEQ ID NO: 667; (d) a CDR-L1 comprising the amino acid sequence set forth in SEQ ID NO: 681; (e) a CDR-L2 comprising the amino acid sequence set forth SEQ ID NO: 695; and/or (f) a CDR-L3 comprising the amino acid sequence set forth SEQ ID NO: 709;
(ii)(a) a CDR-H1 comprising the amino acid sequence set forth in SEQ ID NO: 472; (b) a CDR-H2 comprising the amino acid sequence set forth in SEQ ID NO: 497; (c) a CDR-H3 comprising the amino acid sequence set forth in SEQ ID NO: 535; (d) a CDR-L1 comprising the amino acid sequence set forth in SEQ ID NO: 560; (e) a CDR-L2 comprising the amino acid sequence set forth SEQ ID NO: 585; and/or (f) a CDR-L3 comprising the amino acid sequence set forth SEQ ID NO: 610;
(iii)(a) a CDR-H1 comprising the amino acid sequence set forth in SEQ ID NO: 470; (b) a CDR-H2 comprising the amino acid sequence set forth in SEQ ID NO: 495; (c) a CDR-H3 comprising the amino acid sequence set forth in SEQ ID NO: 533; (d) a CDR-L1 comprising the amino acid sequence set forth in SEQ ID NO: 558; (e) a CDR-L2 comprising the amino acid sequence set forth SEQ ID NO: 583; and/or (f) a CDR-L3 comprising the amino acid sequence set forth SEQ ID NO: 608;
(iv)(a) a CDR-H1 comprising the amino acid sequence set forth in SEQ ID NO: 471; (b) a CDR-H2 comprising the amino acid sequence set forth in SEQ ID NO: 496; (c) a CDR-H3 comprising the amino acid sequence set forth in SEQ ID NO: 534; (d) a CDR-L1 comprising the amino acid sequence set forth in SEQ ID NO: 559; (e) a CDR-L2 comprising the amino acid sequence set forth SEQ ID NO: 584; and/or (f) a CDR-L3 comprising the amino acid sequence set forth SEQ ID NO: 609;
(v)(a) a CDR-H1 comprising the amino acid sequence set forth in SEQ ID NO: 473; (b) a CDR-H2 comprising the amino acid sequence set forth in SEQ ID NO: 498; (c) a CDR-H3 comprising the amino acid sequence set forth in SEQ ID NO: 536; (d) a CDR-L1 comprising the amino acid sequence set forth in SEQ ID NO: 561; (e) a CDR-L2 comprising the amino acid sequence set forth SEQ ID NO: 586; and/or (f) a CDR-L3 comprising the amino acid sequence set forth SEQ ID NO: 611;
(vi)(a) a CDR-H1 comprising the amino acid sequence set forth in SEQ ID NO: 474; (b) a CDR-H2 comprising the amino acid sequence set forth in SEQ ID NO: 499; (c) a CDR-H3 comprising the amino acid sequence set forth in SEQ ID NO: 537; (d) a CDR-L1 comprising the amino acid sequence set forth in SEQ ID NO: 562; (e) a CDR-L2 comprising the amino acid sequence set forth SEQ ID NO: 587; and/or (f) a CDR-L3 comprising the amino acid sequence set forth SEQ ID NO: 612; or
(vii)(a) a CDR-H1 comprising the amino acid sequence set forth in SEQ ID NO: 632; (b) a CDR-H2 comprising the amino acid sequence set forth in SEQ ID NO: 646; (c) a CDR-H3 comprising the amino acid sequence set forth in SEQ ID NO: 660; (d) a CDR-L1 comprising the amino acid sequence set forth in SEQ ID NO: 674; (e) a CDR-L2 comprising the amino acid sequence set forth SEQ ID NO: 688; and/or (f) a CDR-L3 comprising the amino acid sequence set forth SEQ ID NO: 702.

46. The pharmaceutical composition of claim 1 or 40, wherein the antibody or antigen binding fragment thereof comprises:
(i)(a) a CDR-H1 comprising the amino acid sequence set forth in SEQ ID NO: 640; (b) a CDR-H2 comprising the amino acid sequence set forth in SEQ ID NO: 654; (c) a CDR-H3 comprising the amino acid sequence set forth in SEQ ID NO: 668; (d) a CDR-L1 comprising the amino acid sequence set forth in SEQ ID NO: 682; (e) a CDR-L2 comprising the amino acid sequence set forth SEQ ID NO: 696; and/or (f) a CDR-L3 comprising the amino acid sequence set forth SEQ ID NO: 710;
(ii)(a) a CDR-H1 comprising the amino acid sequence set forth in SEQ ID NO: 477; (b) a CDR-H2 comprising the amino acid sequence set forth in SEQ ID NO: 515; (c) a CDR-H3 comprising the amino acid sequence set forth in SEQ ID NO: 540; (d) a CDR-L1 comprising the amino acid sequence set forth in SEQ ID NO: 565; (e) a CDR-L2 comprising the amino acid sequence set forth SEQ ID NO: 590; and/or (f) a CDR-L3 comprising the amino acid sequence set forth SEQ ID NO: 615;
(iii)(a) a CDR-H1 comprising the amino acid sequence set forth in SEQ ID NO: 475; (b) a CDR-H2 comprising the amino acid sequence set forth in SEQ ID NO: 513; (c) a CDR-H3 comprising the amino acid sequence set forth in SEQ ID NO: 538; (d) a CDR-L1 comprising the amino acid sequence set forth in SEQ ID NO: 563; (e) a CDR-L2 comprising the amino acid sequence set forth SEQ ID NO: 588; and/or (f) a CDR-L3 comprising the amino acid sequence set forth SEQ ID NO: 613;
(iv)(a) a CDR-H1 comprising the amino acid sequence set forth in SEQ ID NO: 476; (b) a CDR-H2 comprising the amino acid sequence set forth in SEQ ID NO: 514; (c) a CDR-H3 comprising the amino acid sequence set forth in SEQ ID NO: 539; (d) a CDR-L1 comprising the amino acid sequence set forth in SEQ ID NO: 564; (e) a CDR-L2 comprising the amino acid sequence set forth SEQ ID NO: 589; and/or (f) a CDR-L3 comprising the amino acid sequence set forth SEQ ID NO: 614;
(v)(a) a CDR-H1 comprising the amino acid sequence set forth in SEQ ID NO: 478; (b) a CDR-H2 comprising the amino acid sequence set forth in SEQ ID NO: 516; (c) a CDR-H3 comprising the amino acid sequence set forth in SEQ ID NO: 541; (d) a CDR-L1 comprising the amino acid sequence set forth in SEQ ID NO: 566; (e) a CDR-L2 comprising the amino acid sequence set forth SEQ ID NO: 591; and/or (f) a CDR-L3 comprising the amino acid sequence set forth SEQ ID NO: 616;
(vi)(a) a CDR-H1 comprising the amino acid sequence set forth in SEQ ID NO: 479; (b) a CDR-H2 comprising the amino acid sequence set forth in SEQ ID NO: 517; (c) a CDR-H3 comprising the amino acid sequence set forth in SEQ ID NO: 542; (d) a CDR-L1 comprising the amino acid sequence set forth in SEQ ID NO: 567; (e) a CDR-L2 comprising the amino acid sequence set forth SEQ ID NO: 592; and/or (f) a CDR-L3 comprising the amino acid sequence set forth SEQ ID NO: 617; or
(vii)(a) a CDR-H1 comprising the amino acid sequence set forth in SEQ ID NO: 633; (b) a CDR-H2 comprising the amino acid sequence set forth in SEQ ID NO: 647; (c) a CDR-H3 comprising the amino acid sequence set forth in SEQ ID NO: 661; (d) a CDR-L1 comprising the amino acid sequence set forth in SEQ ID NO: 675; (e) a CDR-L2 comprising the amino acid sequence set forth SEQ ID NO: 689; and/or (f) a CDR-L3 comprising the amino acid sequence set forth SEQ ID NO: 703.

47. The pharmaceutical composition of claim 1 or 40, wherein the antibody or antigen binding fragment thereof comprises:
(i)(a) a CDR-H1 comprising the amino acid sequence set forth in SEQ ID NO: 641; (b) a CDR-H2 comprising the amino acid sequence set forth in SEQ ID NO: 655; (c) a CDR-H3 comprising the amino acid sequence set forth in SEQ ID NO: 669; (d) a CDR-L1 comprising the amino acid sequence set forth in SEQ ID NO: 683; (e) a CDR-L2 comprising the amino acid sequence set forth SEQ ID NO: 697; and/or (f) a CDR-L3 comprising the amino acid sequence set forth SEQ ID NO: 711;
(ii)(a) a CDR-H1 comprising the amino acid sequence set forth in SEQ ID NO: 487; (b) a CDR-H2 comprising the amino acid sequence set forth in SEQ ID NO: 525; (c) a CDR-H3 comprising the amino acid sequence set forth in SEQ ID NO: 550; (d) a CDR-L1 comprising the amino acid sequence set forth in SEQ ID NO: 575; (e) a CDR-L2 comprising the amino acid sequence set forth SEQ ID NO: 600; and/or (f) a CDR-L3 comprising the amino acid sequence set forth SEQ ID NO: 625;
(iii)(a) a CDR-H1 comprising the amino acid sequence set forth in SEQ ID NO: 485; (b) a CDR-H2 comprising the amino acid sequence set forth in SEQ ID NO: 523; (c) a CDR-H3 comprising the amino acid sequence set forth in SEQ ID NO: 548; (d) a CDR-L1 comprising the amino acid sequence set forth in SEQ ID NO: 573; (e) a CDR-L2 comprising the amino acid sequence set forth SEQ ID NO: 598; and/or (f) a CDR-L3 comprising the amino acid sequence set forth SEQ ID NO: 623;
(iv)(a) a CDR-H1 comprising the amino acid sequence set forth in SEQ ID NO: 486; (b) a CDR-H2 comprising the amino acid sequence set forth in SEQ ID NO: 524; (c) a CDR-H3 comprising the amino acid sequence set forth in SEQ ID NO: 549; (d) a CDR-L1 comprising the amino acid sequence set forth in SEQ ID NO: 574; (e) a CDR-L2 comprising the amino acid sequence set forth SEQ ID NO: 599; and/or (f) a CDR-L3 comprising the amino acid sequence set forth SEQ ID NO: 624;
(v)(a) a CDR-H1 comprising the amino acid sequence set forth in SEQ ID NO: 488; (b) a CDR-H2 comprising the amino acid sequence set forth in SEQ ID NO: 526; (c) a CDR-H3 comprising the amino acid sequence set forth in SEQ ID NO: 551; (d) a CDR-L1 comprising the amino acid sequence set forth in SEQ ID NO: 576; (e) a CDR-L2 comprising the amino acid sequence set forth SEQ ID NO: 601; and/or (f) a CDR-L3 comprising the amino acid sequence set forth SEQ ID NO: 626;
(vi)(a) a CDR-H1 comprising the amino acid sequence set forth in SEQ ID NO: 489; (b) a CDR-H2 comprising the amino acid sequence set forth in SEQ ID NO: 527; (c) a CDR-H3 comprising the amino acid sequence set forth in SEQ ID NO: 552; (d) a CDR-L1 comprising the amino acid sequence set forth in SEQ ID NO: 577; (e) a CDR-L2 comprising the amino acid sequence set forth SEQ ID NO: 602; and/or (f) a CDR-L3 comprising the amino acid sequence set forth SEQ ID NO: 627; or
(vii)(a) a CDR-H1 comprising the amino acid sequence set forth in SEQ ID NO: 634; (b) a CDR-H2 comprising the amino acid sequence set forth in SEQ ID NO: 648; (c) a CDR-H3 comprising the amino acid sequence set forth in SEQ ID NO: 662; (d) a CDR-L1 comprising the amino acid sequence set forth in SEQ ID NO: 676; (e) a CDR-L2 comprising the amino acid sequence set forth SEQ ID NO: 690; and/or (f) a CDR-L3 comprising the amino acid sequence set forth SEQ ID NO: 704.

48. The pharmaceutical composition of any one of claims 1 to 47, wherein the antibody or antigen binding fragment thereof comprises:
(i)(a) an immunoglobulin variable region heavy chain (VH) comprising an amino acid sequence having at least about 90, 95, 97, 98, 99, or 100% sequence identity to any one of SEQ ID NOs: 1, 2, 5, 6, 9, 10, 13, 14, 17, 19, 21, 23, 25, 27, 29, and 31; and/or (b) an immunoglobulin variable region light chain (VL) comprising an amino acid sequence having at least about 90, 95, 97, 98, 99, or 100% sequence identity to any one of SEQ ID NOs: 3, 4, 7, 8, 11, 12, 15, 16, 18, 20, 22, 24, 26, 28, and 30;
(ii)(a) a VH comprising an amino acid sequence having at least about 90, 95, 97, 98, 99, or 100% sequence identity to SEQ ID NO: 1; and/or (b) a VL comprising an amino acid sequence having at least about 90, 95, 97, 98, 99, or 100% sequence identity to SEQ ID NO: 3;
(iii)(a) a VH comprising an amino acid sequence having at least about 90, 95, 97, 98, 99, or 100% sequence identity to SEQ ID NO: 2; and/or (b) a VL comprising an amino acid sequence having at least about 90, 95, 97, 98, 99, or 100% sequence identity to SEQ ID NO: 4;
(iv)(a) a VH comprising an amino acid sequence having at least about 90, 95, 97, 98, 99, or 100% sequence identity to SEQ ID NO: 5; and/or (b) a VL comprising an amino acid sequence having at least about 90, 95, 97, 98, 99, or 100% sequence identity to SEQ ID NO: 7;
(v)(a) a VH comprising an amino acid sequence having at least about 90, 95, 97, 98, 99, or 100% sequence identity to SEQ ID NO: 6; and/or (b) a VL comprising an amino acid sequence having at least about 90, 95, 97, 98, 99, or 100% sequence identity to SEQ ID NO: 8;
(vi)(a) a VH comprising an amino acid sequence having at least about 90, 95, 97, 98, 99, or 100% sequence identity to SEQ ID NO: 9; and/or (b) a VL comprising an amino acid sequence having at least about 90, 95, 97, 98, 99, or 100% sequence identity to SEQ ID NO: 11;
(vii)(a) a VH comprising an amino acid sequence having at least about 90, 95, 97, 98, 99, or 100% sequence identity to SEQ ID NO: 10; and/or (b) a VL comprising an amino acid sequence having at least about 90, 95, 97, 98, 99, or 100% sequence identity to SEQ ID NO: 12;
(viii)(a) a VH comprising an amino acid sequence having at least about 90, 95, 97, 98, 99, or 100% sequence identity to SEQ ID NO: 13; and/or (b) a VL comprising an amino acid sequence having at least about 90, 95, 97, 98, 99, or 100% sequence identity to SEQ ID NO: 15;
(ix)(a) a VH comprising an amino acid sequence having at least about 90, 95, 97, 98, 99, or 100% sequence identity to SEQ ID NO: 14; and/or (b) a VL comprising an amino acid sequence having at least about 90, 95, 97, 98, 99, or 100% sequence identity to SEQ ID NO: 16;
(x)(a) a VH comprising an amino acid sequence having at least about 90, 95, 97, 98, 99, or 100% sequence identity to SEQ ID NO: 23; and/or (b) a VL comprising an amino acid sequence having at least about 90, 95, 97, 98, 99, or 100% sequence identity to SEQ ID NO: 24;
(xi)(a) a VH comprising an amino acid sequence having at least about 90, 95, 97, 98, 99, or 100% sequence identity to SEQ ID NO: 25; and/or (b) a VL comprising an amino acid sequence having at least about 90, 95, 97, 98, 99, or 100% sequence identity to SEQ ID NO: 26;
(xii)(a) a VH comprising an amino acid sequence having at least about 90, 95, 97, 98, 99, or 100% sequence identity to SEQ ID NO: 27; and/or (b) a VL comprising an amino acid sequence having at least about 90, 95, 97, 98, 99, or 100% sequence identity to SEQ ID NO: 28;
(xiii)(a) a VH comprising an amino acid sequence having at least about 90, 95, 97, 98, 99, or 100% sequence identity to SEQ ID NO: 29; and/or (b) a VL comprising an amino acid sequence having at least about 90, 95, 97, 98, 99, or 100% sequence identity to SEQ ID NO: 30; or
(xiv)(a) a VH comprising an amino acid sequence having at least about 90, 95, 97, 98, 99, or 100% sequence identity to SEQ ID NO: 31; and/or (b) a VL comprising an amino acid sequence having at least about 90, 95, 97, 98, 99, or 100% sequence identity to SEQ ID NO: 32.

49. The pharmaceutical composition of any one of claims 1, 40 and 48, wherein the antibody or antigen binding fragment thereof comprises:
(i)(a) a VH comprising an amino acid sequence set forth in any one of SEQ ID NOs: 1, 2, 5, 6, 9, 10, 13, 14, 17, 19, 21, 23, 25, 27, 29, and 31; and/or (b) a VL comprising an amino acid sequence set forth in any one of SEQ ID NOs: 3, 4, 7, 8, 11, 12, 15, 16, 18, 20, 22, 24, 26, 28, and 30;
(ii)(a) a VH comprising an amino acid sequence set forth in SEQ ID NO: 1; and/or (b) a VL comprising an amino acid sequence set forth in SEQ ID NO: 3;
(iii)(a) a VH comprising an amino acid sequence set forth in SEQ ID NO: 2; and/or (b) a VL comprising an amino acid sequence set forth in SEQ ID NO: 4;
(iv)(a) a VH comprising an amino acid sequence set forth in SEQ ID NO: 5; and/or (b) a VL comprising an amino acid sequence set forth in SEQ ID NO: 7;
(v)(a) a VH comprising an amino acid sequence set forth in SEQ ID NO: 6; and/or (b) a VL comprising an amino acid sequence set forth in SEQ ID NO: 8;
(vi)(a) a VH comprising an amino acid sequence set forth in SEQ ID NO: 9; and/or (b) a VL comprising an amino acid sequence set forth in SEQ ID NO: 11;
(vii)(a) a VH comprising an amino acid sequence set forth in SEQ ID NO: 10; and/or (b) a VL comprising an amino acid sequence set forth in SEQ ID NO: 12;
(viii)(a) a VH comprising an amino acid sequence set forth in SEQ ID NO: 13; and/or (b) a VL comprising an amino acid sequence set forth in SEQ ID NO: 15;
(ix)(a) a VH comprising an amino acid sequence set forth in SEQ ID NO: 14; and/or (b) a VL comprising an amino acid sequence set forth in SEQ ID NO: 16;
(x)(a) a VH comprising an amino acid sequence set forth in SEQ ID NO: 23; and/or (b) a VL comprising an amino acid sequence set forth in SEQ ID NO: 24;
(xi)(a) a VH comprising an amino acid sequence set forth in SEQ ID NO: 25; and/or (b) a VL comprising an amino acid sequence set forth in SEQ ID NO: 26;
(xii)(a) a VH comprising an amino acid sequence set forth in SEQ ID NO: 27; and/or (b) a VL comprising an amino acid sequence set forth in SEQ ID NO: 28;
(xiii)(a) a VH comprising an amino acid sequence set forth in SEQ ID NO: 29; and/or (b) a VL comprising an amino acid sequence set forth in SEQ ID NO: 30; or
(xiv)(a) a VH comprising an amino acid sequence set forth in SEQ ID NO: 31; and/or (b) a VL comprising an amino acid sequence set forth in SEQ ID NO: 32.

50. The pharmaceutical composition of any one of claims 1 to 49, the antibody or antigen binding fragment thereof further comprising an IgG constant region.

51. The pharmaceutical composition of any one of claims 1 to 50, the antibody or antigen binding fragment thereof further comprising an IgG constant region having reduced antibody-dependent cell-mediated cytotoxicity (ADCC) function as compared to human IgG and/or reduced complement-dependent cytotoxicity (CDC) as compared to human IgG.

52. The pharmaceutical composition of claim 50 or 51, wherein the constant region comprises an amino acid sequence having 80, 85, 90, 95, 97, 98, 99, or 100% sequence identity to the amino acid sequence set forth by any one of SEQ ID NOs: 500-512.

53. The pharmaceutical composition of any one of claims 50 to 52, wherein the constant region comprises the amino acid sequence set forth by any one of SEQ ID NOs: 500-512.

54. The pharmaceutical composition of any one of claims 1 to 53, wherein the antibody or antigen binding fragment thereof comprises a constant region having an amino acid sequence variant corresponding to (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) E233P, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) any combination of (a)-(mmm), per EU numbering.

55. The pharmaceutical composition of any one of claims 1 to 54, wherein the antibody or antigen binding fragment thereof is an IgG antibody.

56. The pharmaceutical composition of claim 55, wherein the IgG antibody is IgG1, IgG2, IgG3, or IgG4.

57. The pharmaceutical composition of any one of claims 1 to 56, wherein the antibody or antigen binding fragment thereof is human, chimeric, or humanized.

58. The pharmaceutical composition of any one of claims 1 to 57, wherein the antibody or antigen binding fragment thereof is a Fab, F(ab′)₂, a single-domain antibody, or a single chain variable fragment (scFv).

59. The pharmaceutical composition of any one of claims 1 to 58, wherein the antibody or antigen binding fragment thereof is a bispecific or multispecific antibody.

60. The pharmaceutical composition of any one of claims 1 to 59, wherein the antibody or antigen binding fragment thereof binds one or more amino acids residues of CD30L that interact with CD30.

61. The pharmaceutical composition of any one of claims 1 to 60, wherein the antibody or antigen binding fragment thereof inhibits a binding interaction between CD30L and CD30.

62. The pharmaceutical composition of any one of claims 1 to 60, wherein the antibody or antigen binding fragment thereof blocks a binding interaction between CD30L and CD30.

63. The pharmaceutical composition of claim 61 or 62, wherein the inhibition or blocking is determined in an ELISA assay, a cell binding assay with CD30L expressing cells, or a surface plasmon resonance (SPR) assay.

64. The pharmaceutical composition of any one of claims 1 to 63, wherein the antibody or antigen binding fragment thereof specifically binds to CD30L.

65. The pharmaceutical composition of any one of claims 1 to 64, wherein the antibody or antigen binding fragment thereof (i) inhibits interleukin-8 secretion in a cell-based assay, (ii) inhibits interleukin-6 secretion in a cell-based assay, or (iii) both (i) and (ii).

66. The pharmaceutical composition of any one of claims 1 to 64, wherein the antibody or antigen binding fragment thereof (i) blocks interleukin-8 secretion in a cell-based assay, (ii) blocks interleukin-6 secretion in a cell-based assay, or (iii) both (i) and (ii).

67. The pharmaceutical composition of claim 65 or 66, wherein the cell-based assay is a dual cell assay with a cell expressing CD30 and a cell expressing CD30L.

68. The pharmaceutical composition of any one of claims 1 to 67, wherein the antibody or antigen binding fragment thereof binds to (i) human CD30L, (ii) cynomolgus CD30L, or (iii) both human CD30L and cynomolgus CD30L.

69. The pharmaceutical composition of any one of claims 1 to 68, wherein the antibody or antigen binding fragment thereof binds to CD30L with a dissociation equilibrium constant (K_{D}) of no more than 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, or 1000 pM.

70. The pharmaceutical composition of any one of claims 1 to 69, wherein the antibody or antigen binding fragment thereof binds to CD30L with an association rate constant (kₒₙ) of at least 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 1.1×10⁶, 1.2×10⁶, 1.3×10⁶, 1.4×10⁶, 1.5×10⁶, or 1.55×10⁶M⁻¹ S⁻¹.

71. The pharmaceutical composition of any one of claims 1 to 70, wherein the antibody or antigen binding fragment thereof binds to CD30L with a dissociation rate constant (k_{off}) of no more than 1.4×10⁻⁴, 1.41×10⁻⁴, 1.5×10⁻⁴, 1.6×10⁻⁴, 1.7×10⁻⁴, 1.8×10⁻⁴, 1.9×10⁻⁴, 2.0×10⁻⁴, 2.1×10⁻⁴, 2.2×10⁻⁴, 2.3×10⁻⁴, 2.4×10⁻⁴, 2.5×10⁻⁴, 2.6×10⁻⁴, 2.7×10⁻⁴, 2.8×10⁻⁴, 2.9×10⁻⁴, 3.0×10⁻⁴, 3.1×10⁻⁴, 3.2×10⁻⁴, 3.3×10⁻⁴, 3.4×10⁻⁴, or 3.5×10⁻⁴ S⁻¹.

72. A pharmaceutical composition comprising an anti-CD30L antibody or antigen binding fragment thereof, wherein the antibody or antigen binding fragment thereof binds isoform 1 of CD30L, and wherein the antibody or antigen binding fragment thereof does not bind to isoform 2 of CD30L.

73. The pharmaceutical composition of any one of claims 1 to 71, wherein the antibody or antigen binding fragment thereof binds isoform 1 of CD30L, and wherein the antibody or antigen binding fragment thereof does not bind to isoform 2 of CD30L.

74. The pharmaceutical composition of claim 72 or 73, wherein the isoform 1 of CD30L comprises the amino acid sequence set forth in SEQ ID NO: 34 and wherein the isoform 2 of CD30L comprises the amino acid sequence set forth in SEQ ID NO: 35.

75. The pharmaceutical composition of any one of claims 1 to 71, wherein the antibody or antigen binding fragment thereof is recombinant antibody or antigen binding fragment thereof.

76. The pharmaceutical composition of any one of claims 1 to 75, wherein the antibody or antigen binding fragment thereof is isolated antibody or antigen binding fragment thereof.

77. The pharmaceutical composition of any one of claims 1 to 76, wherein the anti-CD30L antibody or antigen binding fragment is (a) at a concentration (i) from about 50 mg/ml to about 275 mg/ml, (ii) from about 50 mg/ml to about 250 mg/ml, (iii) from about 50 mg/ml to about 225 mg/ml, (iv) from about 50 mg/ml to about 200 mg/ml, (v) from about 50 mg/ml to about 175 mg/ml, (vi) from about 50 mg/ml to about 150 mg/ml, (vii) from about 50 mg/ml to about 125 mg/ml, (viii) from about 50 mg/ml to about 100 mg/ml, or (ix) from about 50 mg/ml to about 75 mg/ml;
(b) at a concentration (i) from about 75 mg/ml to about 275 mg/ml, (ii) from about 75 mg/ml to about 250 mg/ml, (iii) from about 75 mg/ml to about 225 mg/ml, (iv) from about 75 mg/ml to about 200 mg/ml, (v) from about 75 mg/ml to about 175 mg/ml, (vi) from about 75 mg/ml to about 150 mg/ml, (vii) from about 75 mg/ml to about 125 mg/ml, or (viii) from about 75 mg/ml to about 100 mg/ml;
(c) at a concentration (i) from about 100 mg/ml to about 275 mg/ml, (ii) from about 100 mg/ml to about 250 mg/ml, (iii) from about 100 mg/ml to about 225 mg/ml, (iv) from about 100 mg/ml to about 200 mg/ml, (v) from about 100 mg/ml to about 175 mg/ml, (vi) from about 100 mg/ml to about 150 mg/ml, or (vii) from about 100 mg/ml to about 125 mg/ml;
(d) at a concentration (i) from about 125 mg/ml to about 275 mg/ml, (ii) from about 125 mg/ml to about 250 mg/ml, (iii) from about 125 mg/ml to about 225 mg/ml, (iv) from about 125 mg/ml to about 200 mg/ml, (v) from about 125 mg/ml to about 175 mg/ml, or (vi) from about 125 mg/ml to about 150 mg/ml;
(e) at a concentration (i) from about 150 mg/ml to about 275 mg/ml, (ii) from about 150 mg/ml to about 250 mg/ml, (iii) from about 150 mg/ml to about 225 mg/ml, (iv) from about 150 mg/ml to about 200 mg/ml, or (v) from about 150 mg/ml to about 175 mg/ml;
(f) at a concentration (i) from about 175 mg/ml to about 275 mg/ml, (ii) from about 175 mg/ml to about 250 mg/ml, (iii) from about 175 mg/ml to about 225 mg/ml, or (iv) from about 175 mg/ml to about 200 mg/ml;
(g) at a concentration (i) from about 200 mg/ml to about 275 mg/ml, (ii) from about 200 mg/ml to about 250 mg/ml, or (iii) from about 200 mg/ml to about 225 mg/ml; or
(h) at a concentration (i) at least about 50 mg/mL, (ii) at least about 75 mg/ml, (iii) at least about 100 mg/ml, (iv) at least about 125 mg/ml, (v) at least about 150 mg/ml, (vi) at least about 175 mg/ml, (vii) at least about 200 mg/ml, (viii) at least about 225 mg/ml, or (ix) at least about 250 mg/ml.

78. A pharmaceutical composition comprising an antibody or antigen binding fragment that binds to CD30L (anti-CD30L antibody or antigen binding fragment) at a concentration (i) at least about 50 mg/mL, (ii) at least about 75 mg/ml, (iii) at least about 100 mg/ml, (iv) at least about 125 mg/ml, (v) at least about 150 mg/ml, (vi) at least about 175 mg/ml, (vii) at least about 200 mg/ml, (viii) at least about 225 mg/ml, or (ix) at least about 250 mg/ml.

79. A pharmaceutical composition comprising an antibody or antigen binding fragment that binds to CD30L (anti-CD30L antibody or antigen binding fragment)
(a) at a concentration (i) from about 50 mg/ml to about 275 mg/ml, (ii) from about 50 mg/ml to about 250 mg/ml, (iii) from about 50 mg/ml to about 225 mg/ml, (iv) from about 50 mg/ml to about 200 mg/ml, (v) from about 50 mg/ml to about 175 mg/ml, (vi) from about 50 mg/ml to about 150 mg/ml, (vii) from about 50 mg/ml to about 125 mg/ml, (viii) from about 50 mg/ml to about 100 mg/ml, or (ix) from about 50 mg/ml to about 75 mg/ml;
(b) at a concentration (i) from about 75 mg/ml to about 275 mg/ml, (ii) from about 75 mg/ml to about 250 mg/ml, (iii) from about 75 mg/ml to about 225 mg/ml, (iv) from about 75 mg/ml to about 200 mg/ml, (v) from about 75 mg/ml to about 175 mg/ml, (vi) from about 75 mg/ml to about 150 mg/ml, (vii) from about 75 mg/ml to about 125 mg/ml, or (viii) from about 75 mg/ml to about 100 mg/ml;
(c) at a concentration (i) from about 100 mg/ml to about 275 mg/ml, (ii) from about 100 mg/ml to about 250 mg/ml, (iii) from about 100 mg/ml to about 225 mg/ml, (iv) from about 100 mg/ml to about 200 mg/ml, (v) from about 100 mg/ml to about 175 mg/ml, (vi) from about 100 mg/ml to about 150 mg/ml, or (vii) from about 100 mg/ml to about 125 mg/ml;
(d) at a concentration (i) from about 125 mg/ml to about 275 mg/ml, (ii) from about 125 mg/ml to about 250 mg/ml, (iii) from about 125 mg/ml to about 225 mg/ml, (iv) from about 125 mg/ml to about 200 mg/ml, (v) from about 125 mg/ml to about 175 mg/ml, or (vi) from about 125 mg/ml to about 150 mg/ml;
(e) at a concentration (i) from about 150 mg/ml to about 275 mg/ml, (ii) from about 150 mg/ml to about 250 mg/ml, (iii) from about 150 mg/ml to about 225 mg/ml, (iv) from about 150 mg/ml to about 200 mg/ml, or (v) from about 150 mg/ml to about 175 mg/ml;
(f) at a concentration (i) from about 175 mg/ml to about 275 mg/ml, (ii) from about 175 mg/ml to about 250 mg/ml, (iii) from about 175 mg/ml to about 225 mg/ml, or (iv) from about 175 mg/ml to about 200 mg/ml; or
(g) at a concentration (i) from about 200 mg/ml to about 275 mg/ml, (ii) from about 200 mg/ml to about 250 mg/ml, or (iii) from about 200 mg/ml to about 225 mg/ml.

80. The pharmaceutical composition of any one of claims 1 to 79, further comprising a buffer.

81. The pharmaceutical composition of claim 80, wherein the buffer is selected from the group consisting of an acetate buffer, a succinate buffer, a histidine buffer, a phosphate buffer, and a citrate buffer.

82. The pharmaceutical composition of claim 80 or 81, wherein the buffer is a histidine buffer.

83. The pharmaceutical composition of any one of claims 80 to 82, wherein the concentration of the buffer is from 0.1 mM to 1 M.

84. The pharmaceutical composition of any one of claims 80 to 83, wherein the concentration of the buffer is from 1 mM to 100 mM.

85. The pharmaceutical composition of any one of claims 80 to 84, wherein the concentration of the buffer is from 10 mM to 50 mM.

86. The pharmaceutical composition of any one of claims 80 to 85, wherein the concentration of the buffer is from about 20 mM.

87. The pharmaceutical composition of any one of claims 80 to 86, wherein the pH of the buffer is from 4 to 7.

88. The pharmaceutical composition of any one of claims 80 to 87, wherein the pH of the buffer is from 5 to 6.

89. The pharmaceutical composition of any one of claims 80 to 88, wherein the pH of the buffer is about 5.5.

90. The pharmaceutical composition of any one of claims 1 to 89, further comprising a pharmaceutically acceptable excipient.

91. The pharmaceutical composition of claim 90, wherein the excipient is arginine, arginine hydrochloride (arginine HCl), proline, a polyol, sodium chloride, glycine, lysine, lysine-hydrochloride, arginine glutamate, potassium chloride, magnesium chloride, calcium chloride, or a combination thereof.

92. The pharmaceutical composition of claim 90 or 91, wherein the excipient is arginine HCl.

93. The pharmaceutical composition of claim 91 or 92, wherein the arginine HCl is
(i) at a concentration from 25 mM to 300 mM,
(ii) at a concentration from 50 mM to 250 mM
(iii) at a concentration from 50 mM to 200 mM,
(iv) at a concentration from 75 mM to 150 mM, or
(v) at a concentration of about 100 mM.

94. The pharmaceutical composition of any one of claims 91 to 93, wherein the polyol is selected from the group consisting of sugar, sugar alcohol, and sugar acid.

95. The pharmaceutical composition of claim 94, wherein the sugar is sucrose.

96. The pharmaceutical composition of claim 95, wherein the sucrose is:
(i) at a concentration from 5% to 10% (weight to volume, w/v),
(ii) at a concentration of about 5% (w/v),
(iii) at a concentration from 150 mM to 300 mM, or
(iv) at a concentration of about 150 mM.

97. The pharmaceutical composition of any one of claims 1 to 96, further comprising a surfactant.

98. The pharmaceutical composition of claim 97, wherein the surfactant is a polysorbate.

99. The pharmaceutical composition of claim 97 or 98, wherein the surfactant is polysorbate-80 (PS80).

100. The pharmaceutical composition of claim 97 or 98, wherein the surfactant is poloxamer 188 (P188).

101. The pharmaceutical composition of claim 97 or 98, wherein the surfactant is polysorbate-20 (PS20).

102. The pharmaceutical composition of any one of claims 97 to 101, wherein the concentration of the surfactant is
(i) from 0.001% to 0.1% (w/v),
(ii) from 0.005 to 0.05% (w/v),
(iii) from 0.01 to 0.05% (w/v),
(iv) from 0.01 to 0.04% (w/v),
(v) from 0.01 to 0.03% (w/v),
(vi) from 0.01 to 0.02% (w/v),
(vii) about 0.02% (w/v),
(viii) about 0.03% (w/v),
(ix) about 0.04% (w/v), or
(x) about 0.05% (w/v).

103. The pharmaceutical composition of any one of claims 1 to 102, comprising sodium chloride (NaCl).

104. The pharmaceutical composition of claim 103, wherein the concentration of NaCl is
(i) from 25 mM to 150 mM,
(ii) from 25 mM to 100 mM
(iii) from 50 mM to 100 mM,
(iv) from 75 mM to 100 mM, or
(v) about 50 mM.

105. The pharmaceutical composition of any one of claims 1 to 104, wherein the pharmaceutical composition comprises an anti-oxidant.

106. The pharmaceutical composition of any one of claims 1 to 104, wherein the pharmaceutical composition does not comprise an anti-oxidant.

107. The pharmaceutical composition of any one of claims 1 to 106, wherein the viscosity of the composition is from 1 to 50 cP at 25° C.

108. The pharmaceutical composition of any one of claims 1 to 107, wherein the viscosity of the composition is from 10 to 25 cP at 25° C.

109. The pharmaceutical composition of any one of claims 1 to 107, wherein the viscosity of the composition is from 1 to 50, 2 to 49, 3 to 48, 4 to 46, 5 to 45, 6 to 44, 7 to 43, 8 to 42, 9 to 41, 10 to 40, 10 to 35, 10 to 30, 11 to 29, 12 to 28, 13 to 27, 14 to 26, 15 to 25, 16 to 24, 17 to 23, 18 to 22, 18 to 21, 18 to 20, 18 to 19, 19 to 21, 1 to 10, 1 to 15, or 1 to 20 cP at 25° C.

110. The pharmaceutical composition of any one of claims 1 to 109, wherein the viscosity of the composition is no more than 25 cP, no more than 24 cP, no more than 23 cP, no more than 22 cP, no more than 21 cP, no more than 20 cP, or no more than 19 cP at 25° C.

111. The pharmaceutical composition of any one of claims 1 to 110, wherein the viscosity of the composition is about 15 cP, about 16 cP, about 17 cP, about 18 cP, about 19 cP, about 20 cP, about 21 cP, about 22 cP, about 23 cP, about 24 cP, or about 25 cP at 25° C.

112. The pharmaceutical composition of any one of claims 1 to 111, wherein the composition is a stable composition.

113. The pharmaceutical composition of any one of claims 1 to 112, wherein the composition is a stable:
(i) for at least 2, 4, 8, 12, 16, 20, or 24 weeks when stored at 25° C.±5° C.,
(ii) for at least 2, 4, or 8 weeks when stored at 40° C.±5° C., or
(iii) for at least 2, 4, 8, 12, 16, 20, or 24 weeks when stored at 25° C.±5° C.

114. The pharmaceutical composition of claim 112 or 113, wherein the stability of the composition is determined by:
(i) maintenance of at least 98% of the antibody main peak in size exclusion chromatography,
(ii) maintenance of at least 65% of the antibody main peak in iCIEF,
(iii) maintenance of at least 98% purity,
(iv) maintenance of at least 99% of the antibody concentration,
(v) maintenance of no more than 500 particles/ml of particles over 2 μm, no more than 10 particles/ml of particles over 10 μm, and/or no more than 2 particles/ml of particles over 25 μm, and/or
(vi) any combination of (i) to (iv).

115. The pharmaceutical composition of any one of claims 1 to 114, wherein the composition comprises the anti-CD30L antibody or antigen binding fragment at a concentration of 200 mg/mL, 20 mM histidine buffer, 100 mM arginine monohydrochloride, and 0.02% (w/v) PS20, at pH 5.5.

116. A pharmaceutical composition comprising an anti-CD30L antibody or antigen binding fragment at a concentration of 200 mg/mL, 20 mM histidine buffer, 100 mM arginine monohydrochloride, and 0.02% (w/v) PS20, at pH 5.5.

117. The pharmaceutical composition of any one of claims 1 to 116, the anti-CD30L blocks both the soluble CD30L and the transmembrane CD30L.

118. A method of making the pharmaceutical formulation of any one of claims 1 to 117, comprising:
culturing a cell of in a medium, wherein the cell comprises one or more polynucleotides comprising nucleotide sequences encoding a heavy chain, a light chain, or both a heavy chain and a light chain of the antibody or antigen-binding fragment thereof.

119. The method of claim 118, further comprising expressing the one or more polynucleotides in the cell.

120. The method of claim 118 or 119, further comprising purifying the antibody or antigen-binding fragment thereof.

121. The method of any one of claims 118-120, further comprising a formulation step.

122. The pharmaceutical composition of any one of claims 1 to 117 for use in a method of inhibiting binding of CD30L to CD30.

123. The pharmaceutical composition of any one of claims 1 to 117 for use in a method of inhibiting activation of CD30 signaling in a cell.

124. The pharmaceutical composition of any one of claims 1 to 117 for use in a method of inhibiting activation, expression, and/or secretion of a pro-inflammatory cytokine protein.

125. The pharmaceutical composition for use of claim 124, wherein the pro-inflammatory cytokine protein is interleukin-8 and/or interleukin-6.

126. The pharmaceutical composition for use of claim 124, wherein the interleukin-8 is expressed or released by a T lymphocyte.

127. The pharmaceutical composition of any one of claims 1 to 117 for use in treating an autoimmune disease in an individual in need thereof.

128. The pharmaceutical composition of claim 127, wherein the autoimmune disease is irritable bowel disease.

129. The pharmaceutical composition of claim 127, wherein the irritable bowel disease comprises ulcerative colitis (UC) or Crohn's Disease (CD).

130. A method of treating or ameliorating an autoimmune disease in an individual in need thereof, the method comprising administering to the individual the pharmaceutical composition of any one of claims 1 to 117, thereby treating or ameliorating the autoimmune disease.

131. The method of claim 130, wherein the autoimmune disease is irritable bowel disease.

132. The method of claim 131, wherein the irritable bowel disease comprises ulcerative colitis (UC) or Crohn's Disease (CD).

133. A method for inhibiting and/or reducing binding of CD30L to CD30 in an individual afflicted with an inflammatory or autoimmune disorder, the method comprising administering to the individual afflicted with the inflammatory or autoimmune disorder the pharmaceutical composition of any one of claims 1 to 117, thereby inhibiting and/or reducing the binding of CD30L to CD30.

134. The method of claim 133, wherein the individual is afflicted with an autoimmune disease.

135. The method of claim 133, wherein the autoimmune disease is irritable bowel disease.

136. The method of claim 135, wherein the irritable bowel disease comprises ulcerative colitis (UC) or Crohn's Disease (CD).

137. A method of reducing and/or inhibiting inflammation in an individual, the method comprising administering to the individual the pharmaceutical composition of any one of claims 1 to 117, thereby reducing and/or inhibiting inflammation.

138. The method of claim 137, wherein reducing and/or inhibiting inflammation comprises reducing an amount of pro-inflammatory cytokine expression or secretion in the individual or a tissue of the individual.

139. The method of claim 137 or 138, wherein reducing and/or inhibiting inflammation comprises reducing an amount of pro-inflammatory cytokine expression or secretion by a T lymphocyte of the individual.

140. The method of claim 137, 138 or 139, wherein the pro-inflammatory cytokine comprises interleukin-8.

141. The method of any one of claims 137 to 140, wherein the individual has an autoimmune disease.

142. The method of claim 141, wherein the autoimmune disease is irritable bowel disease.

143. The method of claim 142, wherein the irritable bowel disease comprises ulcerative colitis (UC) or Crohn's Disease (CD).


SEQUENCE LISTING (824 sequences)

SEQ ID NO: 1 - protein, 115 aa, synthetic construct
QVQLQESGPGLVKPSETLSLTCTVSGGSISGYYWSWIRQPPVKGLEWIGYIFYSGYTKYNPSLKSRVTISVDTSKNQFSLKLSSVTAADTAVYYCARDRWDFDYWGQGALVTVSS

SEQ ID NO: 2 - protein, 115 aa, synthetic construct
QVQLQESGPGLVKPSETLSLTCTVSGGSISGYYWSWIRQPPGKGLEWIGYIFYSGYTKYNPSLKSRVTISVDTSKNQFSLKLSSVTAADTAVYYCARDRWDFDYWGQGTLVTVSS

SEQ ID NO: 3 - protein, 107 aa, synthetic construct
AIQMTQSPSYLSASVGDRVTIACRASQGIRNNLGWYQQKPGKAPKLLIYAESSLQSGVPSRFSGSGSGTDFTLTISSLQPEDFATYYCLQDFNYPYTFGQGTKLEIK

SEQ ID NO: 4 - protein, 107 aa, synthetic construct
AIQMTQSPSSLSASVGDRVTITCRASQGIRNNLGWYQQKPGKAPKLLIYAESSLQSGVPSRFSGSGSGTDFTLTISSLQPEDFATYYCLQDFNYPYTFGQGTKLEIK

SEQ ID NO: 5 - protein, 124 aa, synthetic construct
EVQLVESGGGLVQPGRSLRLSCAASGFNFDDYAMHWVRQAPGKGLEWVSGISWRSGNIGYAGSVKGRFTISRDNAKNSLYLQMNSLRPEDSALYYCAKDKGIGFNWNYEGFDYWGQGTLVTVSS

SEQ ID NO: 6 - protein, 124 aa, synthetic construct
  CROSSLNK 49: note = S is crosslinked to S of SEQ ID NO: 34
  CROSSLNK 108: note = Y is crosslinked to S of SEQ ID NO: 34
  CROSSLNK 35: note = H is crosslinked to R of SEQ ID NO: 34
  CROSSLNK 113: note = Y is crosslinked to K of SEQ ID NO: 34
  CROSSLNK 55: note = S is crosslinked to R of SEQ ID NO: 34
  CROSSLNK 55: note = S is crosslinked to Y of SEQ ID NO: 34
EVQLVESGGGLVQPGRSLRLSCAASGFNFDDYAMHWVRQAPGKGLEWVSGISWRSGNIGYADSVKGRFTISRDNAKNSLYLQMNSLRAEDTALYYCAKDKGIGFNWNYEGFDYWGQGTLVTVSS

SEQ ID NO: 7 - protein, 107 aa, synthetic construct
DIQMTQSPSSLSASVGDRVTITCRASQGISNHLAWFQQKPGKAPKSLIYAASSLQSGVPSKFSGSGSGTDFTLTISSLQPEDFATYYCQQYVTYPLTFGGGTKVEIK

SEQ ID NO: 8 - protein, 107 aa, synthetic construct
  CROSSLNK 93: note = T is crosslinked to K of SEQ ID NO: 34
  CROSSLNK 52: note = S is crosslinked to S of SEQ ID NO: 34
  CROSSLNK 56: note = S is crosslinked to K of SEQ ID NO: 34
DIQMTQSPSSLSASVGDRVTITCRASQGISNHLAWFQQKPGKAPKSLIYAASSLQSGVPSRFSGSGSGTDFTLTISSLQPEDFATYYCQQYVTYPLTFGGGTKVEIK

SEQ ID NO: 9 - protein, 115 aa, synthetic construct; identical to SEQ ID NO: 1

SEQ ID NO: 10 - protein, 115 aa, synthetic construct; identical to SEQ ID NO: 2

SEQ ID NO: 11 - protein, 112 aa, synthetic construct
DIVMTQSPLSLPVTPGEPASFSCRSSQSLLHSNGYNYLDWYLQKPGQSPQLLIYLGSNRASGVPDRFSGSGSGTDFTLKISRVEAADVGVYYCMQALQTPRTFGQGTKVEIK

SEQ ID NO: 12 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPRTFGQGTKVEIK

SEQ ID NO: 13 - protein, 122 aa, synthetic construct
QVQLVESGGGVVQPGRSLRLSCAASGFPFSSYGMHWVRQAPGKGLEWVTFIWFDGNNKDYADSVKGRFSVSRDNSKNTLYLQMNSLRADDTAVYYCARNGVYYGSGAYVDYWGQGTLVTVSS

SEQ ID NO: 14 - protein, 122 aa, synthetic construct
QVQLVESGGGVVQPGRSLRLSCAASGFPFSSYGMHWVRQAPGKGLEWVTFIWFDGNNKDYADSVKGRFTISRDNSKNTLYLQMNSLRAEDTAVYYCARNGVYYGSGAYVDYWGQGTLVTVSS

SEQ ID NO: 15 - protein, 112 aa, synthetic construct; identical to SEQ ID NO: 11

SEQ ID NO: 16 - protein, 112 aa, synthetic construct; identical to SEQ ID NO: 12

SEQ ID NO: 17 - protein, 116 aa, synthetic construct
QVQLVESGGGVVQPGRSLRLSCAASGFTFSIYGMHWVRQAPGKGLEWVAVIWYDGSNKYYVDSVKGRFTISRDNSKNSLYLQMNSLRAEDTAVYYCAREGNLFDYWGQGTLVTVSS

SEQ ID NO: 18 - protein, 106 aa, synthetic construct
DIQMTQSPSTLSASVGDRVTITCRASQSISSWLAWYQQKPGKAPKLLIYKASSLESGVPSRFSGSGSGTEFTLTISSLKPDDFATYYCQQYKSYYTFGQGTKLEIK

SEQ ID NO: 19 - protein, 122 aa, synthetic construct
QVHLVESGGGVVQPGRSLRLSCAASGFAFNIYGMHWVRQAPGKGLEWVAVISYDGSNKVYADSVKGRFSISRDNSKNTLYLQMNSLRAEDTAVYYCAKSGGITMVRGVFDYWGQGTLVTVSS

SEQ ID NO: 20 - protein, 107 aa, synthetic construct
DIQMTQSPSSLPASVGDRVTITCRASQGIRNDLGWYQQKPGKAPKRLIHAASSLHSGVPSRFSGSGSGTEFTLTISSLQPEDFATYYCLQHNRYPITFGQGTRLEIK

SEQ ID NO: 21 - protein, 125 aa, synthetic construct
QITLKESGPTLVKPTQTLTLTCTFSGFSLSTSGVGVGWIRQPPGKALEWLALIYWNDDKRYSPSLKSRLTITKDTSKNQVVLTMTNMDPVDTATYYCAHRRTTTVTIYYYYMDVWGKGTTVTVSS

SEQ ID NO: 22 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPFTFGPGTKVDIK

SEQ ID NO: 23 - protein, 122 aa, synthetic construct
EVQLVESGGGLVQPGGSLRLSCAASGFMFSSYSMNWVRQAPGKGLEWVSYISSSSSTIYDADSVKGRFTISRDDAKNSLYLQMNSLRDEDTAVYYCTREAYPGYYYNYMDVWGKGTTVTVSS

SEQ ID NO: 24 - protein, 107 aa, synthetic construct
DIQMTQSPSSLSASVGDRVTITCRASQSISSRLAWYQQKPGKAPNLLIYKASNLESGVPSRFSGSGSGTEFTLTISSLQPDDFATYYCQQYNSYSRTFGQGTKVEIK

SEQ ID NO: 25 - protein, 118 aa, synthetic construct
EVQLVESGGGLVQPGGSLRLSCAASGFTFSNYAMTWVRQAPGKGLEWVSAISGFGGSTYYADSVKGRFTISRDNSKNTLYLQMNSLRAEDTAVYYCAKDHDYYAFDYWGQGTLVTVSS

SEQ ID NO: 26 - protein, 107 aa, synthetic construct
DIQMTQSPSSLSASVGDRVTITCRASQSISSYLNWYQQKPGKAPKLLIYAVSSLQSGVPSRFSGSGSGTDFTLTISSLQPEDFATYYCQQSYSTPYTFGQGTKLEIK

SEQ ID NO: 27 - protein, 118 aa, synthetic construct
EVQLVESGGGLVQPGGSLKLSCAASGFTFSNYAMTWVRQAPGKGLEWVSAISGYGGSTYYAASVKGRFTISRDNSKNTLFLQMNSLRAEDTAVYYCAKDHDYYAFDYWGQGTLVPVSS

SEQ ID NO: 28 - protein, 107 aa, synthetic construct
DIQMTQSPSSLSASVGDRVTITCRASQSINSYLNWYQQKPGKAPKLLIYSASSLQSGVPSRFSGSGSGTDFTLTISSLQPEDFATYYCQQSYSTPYTFGQGTKLEIK

SEQ ID NO: 29 - protein, 120 aa, synthetic construct
EVQLVESGGGLVQPGGSLRLSCEVSGFTFSSYDMHWVRQVTGTGLEWVSSIGIGGDTYYPGSVKGRFTISRENAKNSLYLQMNSLRGGDTGVYYCARGEWDLLWYFMDVWGKGTTVTVSS

SEQ ID NO: 30 - protein, 107 aa, synthetic construct
DIQMTQSPSSLSASIGDRVTITCRASQGISSWLAWYQQKPGKAPKLLIYAASSLQSGVPSRFSGSGSGTDFTLTISSLQPEDFATYYCQQANSFPLTFGGGTKVEIK

SEQ ID NO: 31 - protein, 125 aa, synthetic construct
EVQLVESGGGLVQPGGSLRLSCAVSGITFSNAWMSWVRQAPGKGLEWVGRIKSKTYGGTTDYAAPVKGRFIISRDDSKDTLYLQMNSLKTEDTAIYYCTTDPWNYVNYNYFMDVWGKGTTVTVSS

SEQ ID NO: 32 - protein, 107 aa, synthetic construct
DIQMTQSPSSLSASVGDRVTITCRASQDIRNYLAWYQQKPGKITHLLVYAASTLQSGVPSRFSGSGSGTDFTLTINSLQPEDVATYYCQNYFSVPLTFGGGTKVEIK

SEQ ID NO: 33 - protein, 234 aa, synthetic construct
MDPGLQQALNGMAPPGDTAMHVPAGSVASHLGTTSRSYFYLTTATLALCLVFTVATIMVLVVQRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 34 - protein, 172 aa, synthetic construct
  CROSSLNK 16: note = K is crosslinked to T of SEQ ID NO: 8
  CROSSLNK 21: note = S is crosslinked to S of SEQ ID NO: 6
  CROSSLNK 21: note = S is crosslinked to S of SEQ ID NO: 8
  CROSSLNK 21: note = S is crosslinked to Y of SEQ ID NO: 6
  CROSSLNK 30: note = R is crosslinked to H of SEQ ID NO: 6
  CROSSLNK 48: note = K is crosslinked to Y of SEQ ID NO: 6
  CROSSLNK 48: note = K is crosslinked to S of SEQ ID NO: 8
  CROSSLNK 63: note = R is crosslinked to S of SEQ ID NO: 6
  CROSSLNK 64: note = Y is crosslinked to S of SEQ ID NO: 6
QRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 35 - protein, 102 aa, synthetic construct
QRTDSIPNSPDNVPLKGGNCSEDLLCILKRAPFKKSWAYLQVAKHLNKTKLSWNKDGILHGVRYQDGNLVIQFPDYCGMILHHSHSTLDSGKGHCCLETLQP

SEQ ID NO: 36-99 - skipped (64 entries)

SEQ ID NO: 100 - protein, 7 aa, synthetic construct
GGSISGY

SEQ ID NO: 101 - protein, 10 aa, synthetic construct
GGSISGYYWS

SEQ ID NO: 102 - protein, 5 aa, synthetic construct
GYYWS

SEQ ID NO: 103 - protein, 6 aa, synthetic construct
SGYYWS

SEQ ID NO: 104 - protein, 8 aa, synthetic construct
GGSISGYY

SEQ ID NO: 105 - protein, 7 aa, synthetic construct
GGSISGY

SEQ ID NO: 106 - protein, 10 aa, synthetic construct
GGSISGYYWS

SEQ ID NO: 107 - protein, 5 aa, synthetic construct
GYYWS

SEQ ID NO: 108 - protein, 6 aa, synthetic construct
SGYYWS

SEQ ID NO: 109 - protein, 8 aa, synthetic construct
GGSISGYY

SEQ ID NO: 110 - protein, 7 aa, synthetic construct
GFNFDDY

SEQ ID NO: 111 - protein, 10 aa, synthetic construct
GFNFDDYAMH

SEQ ID NO: 112 - protein, 5 aa, synthetic construct
DYAMH

SEQ ID NO: 113 - protein, 6 aa, synthetic construct
DDYAMH

SEQ ID NO: 114 - protein, 8 aa, synthetic construct
GFNFDDYA

SEQ ID NO: 115 - protein, 7 aa, synthetic construct
GFNFDDY

SEQ ID NO: 116 - protein, 10 aa, synthetic construct
GFNFDDYAMH

SEQ ID NO: 117 - protein, 5 aa, synthetic construct
DYAMH

SEQ ID NO: 118 - protein, 6 aa, synthetic construct
DDYAMH

SEQ ID NO: 119 - protein, 8 aa, synthetic construct
GFNFDDYA

SEQ ID NO: 120 - protein, 7 aa, synthetic construct
GGSISGY

SEQ ID NO: 121 - protein, 10 aa, synthetic construct
GGSISGYYWS

SEQ ID NO: 122 - protein, 5 aa, synthetic construct
GYYWS

SEQ ID NO: 123 - protein, 6 aa, synthetic construct
SGYYWS

SEQ ID NO: 124 - protein, 8 aa, synthetic construct
GGSISGYY

SEQ ID NO: 125 - protein, 7 aa, synthetic construct
GGSISGY

SEQ ID NO: 126 - protein, 10 aa, synthetic construct
GGSISGYYWS

SEQ ID NO: 127 - protein, 5 aa, synthetic construct
GYYWS

SEQ ID NO: 128 - protein, 6 aa, synthetic construct
SGYYWS

SEQ ID NO: 129 - protein, 8 aa, synthetic construct
GGSISGYY

SEQ ID NO: 130 - protein, 7 aa, synthetic construct
GFPFSSY

SEQ ID NO: 131 - protein, 10 aa, synthetic construct
GFPFSSYGMH

SEQ ID NO: 132 - protein, 5 aa, synthetic construct
SYGMH

SEQ ID NO: 133 - protein, 6 aa, synthetic construct
SSYGMH

SEQ ID NO: 134 - protein, 8 aa, synthetic construct
GFPFSSYG

SEQ ID NO: 135 - protein, 7 aa, synthetic construct
GFPFSSY

SEQ ID NO: 136 - protein, 10 aa, synthetic construct
GFPFSSYGMH

SEQ ID NO: 137 - protein, 5 aa, synthetic construct
SYGMH

SEQ ID NO: 138 - protein, 6 aa, synthetic construct
SSYGMH

SEQ ID NO: 139 - protein, 8 aa, synthetic construct
GFPFSSYG

SEQ ID NO: 140 - protein, 5 aa, synthetic construct
FYSGY

SEQ ID NO: 141 - protein, 9 aa, synthetic construct
YIFYSGYTK

SEQ ID NO: 142 - protein, 16 aa, synthetic construct
YIFYSGYTKYNPSLKS

SEQ ID NO: 143 - protein, 12 aa, synthetic construct
WIGYIFYSGYTK

SEQ ID NO: 144 - protein, 7 aa, synthetic construct
IFYSGYT

SEQ ID NO: 145 - protein, 5 aa, synthetic construct
FYSGY

SEQ ID NO: 146 - protein, 9 aa, synthetic construct
YIFYSGYTK

SEQ ID NO: 147 - protein, 16 aa, synthetic construct
YIFYSGYTKYNPSLKS

SEQ ID NO: 148 - protein, 12 aa, synthetic construct
WIGYIFYSGYTK

SEQ ID NO: 149 - protein, 7 aa, synthetic construct
IFYSGYT

SEQ ID NO: 150 - protein, 6 aa, synthetic construct
SWRSGN

SEQ ID NO: 151 - protein, 10 aa, synthetic construct
GISWRSGNIG

SEQ ID NO: 152 - protein, 17 aa, synthetic construct
GISWRSGNIGYAGSVKG

SEQ ID NO: 153 - protein, 13 aa, synthetic construct
WVSGISWRSGNIG

SEQ ID NO: 154 - protein, 8 aa, synthetic construct
ISWRSGNI

SEQ ID NO: 155 - protein, 6 aa, synthetic construct
SWRSGN

SEQ ID NO: 156 - protein, 10 aa, synthetic construct
GISWRSGNIG

SEQ ID NO: 157 - protein, 17 aa, synthetic construct
GISWRSGNIGYADSVKG

SEQ ID NO: 158 - protein, 13 aa, synthetic construct
WVSGISWRSGNIG

SEQ ID NO: 159 - protein, 8 aa, synthetic construct
ISWRSGNI

SEQ ID NO: 160 - protein, 5 aa, synthetic construct
FYSGY

SEQ ID NO: 161 - protein, 9 aa, synthetic construct
YIFYSGYTK

SEQ ID NO: 162 - protein, 16 aa, synthetic construct
YIFYSGYTKYNPSLKS

SEQ ID NO: 163 - protein, 12 aa, synthetic construct
WIGYIFYSGYTK

SEQ ID NO: 164 - protein, 7 aa, synthetic construct
IFYSGYT

SEQ ID NO: 165 - protein, 5 aa, synthetic construct
FYSGY

SEQ ID NO: 166 - protein, 9 aa, synthetic construct
YIFYSGYTK

SEQ ID NO: 167 - protein, 16 aa, synthetic construct
YIFYSGYTKYNPSLKS

SEQ ID NO: 168 - protein, 12 aa, synthetic construct
WIGYIFYSGYTK

SEQ ID NO: 169 - protein, 7 aa, synthetic construct
IFYSGYT

SEQ ID NO: 170 - protein, 6 aa, synthetic construct
WFDGNN

SEQ ID NO: 171 - protein, 10 aa, synthetic construct
FIWFDGNNKD

SEQ ID NO: 172 - protein, 17 aa, synthetic construct
FIWFDGNNKDYADSVKG

SEQ ID NO: 173 - protein, 13 aa, synthetic construct
WVTFIWFDGNNKD

SEQ ID NO: 174 - protein, 8 aa, synthetic construct
IWFDGNNK

SEQ ID NO: 175 - protein, 6 aa, synthetic construct
WFDGNN

SEQ ID NO: 176 - protein, 10 aa, synthetic construct
FIWFDGNNKD

SEQ ID NO: 177 - protein, 17 aa, synthetic construct
FIWFDGNNKDYADSVKG

SEQ ID NO: 178 - protein, 13 aa, synthetic construct
WVTFIWFDGNNKD

SEQ ID NO: 179 - protein, 8 aa, synthetic construct
IWFDGNNK

SEQ ID NO: 180 - protein, 7 aa, synthetic construct
DRWDFDY

SEQ ID NO: 181 - protein, 7 aa, synthetic construct
DRWDFDY

SEQ ID NO: 182 - protein, 7 aa, synthetic construct
DRWDFDY

SEQ ID NO: 183 - protein, 8 aa, synthetic construct
ARDRWDFD

SEQ ID NO: 184 - protein, 9 aa, synthetic construct
ARDRWDFDY

SEQ ID NO: 185 - protein, 7 aa, synthetic construct
DRWDFDY

SEQ ID NO: 186 - protein, 7 aa, synthetic construct
DRWDFDY

SEQ ID NO: 187 - protein, 7 aa, synthetic construct
DRWDFDY

SEQ ID NO: 188 - protein, 8 aa, synthetic construct
ARDRWDFD

SEQ ID NO: 189 - protein, 9 aa, synthetic construct
ARDRWDFDY

SEQ ID NO: 190 - protein, 15 aa, synthetic construct
DKGIGFNWNYEGFDY

SEQ ID NO: 191 - protein, 15 aa, synthetic construct
DKGIGFNWNYEGFDY

SEQ ID NO: 192 - protein, 15 aa, synthetic construct
DKGIGFNWNYEGFDY

SEQ ID NO: 193 - protein, 16 aa, synthetic construct
AKDKGIGFNWNYEGFD

SEQ ID NO: 194 - protein, 17 aa, synthetic construct
AKDKGIGFNWNYEGFDY

SEQ ID NO: 195 - protein, 15 aa, synthetic construct
DKGIGFNWNYEGFDY

SEQ ID NO: 196 - protein, 15 aa, synthetic construct
DKGIGFNWNYEGFDY

SEQ ID NO: 197 - protein, 15 aa, synthetic construct
DKGIGFNWNYEGFDY

SEQ ID NO: 198 - protein, 16 aa, synthetic construct
AKDKGIGFNWNYEGFD

SEQ ID NO: 199 - protein, 17 aa, synthetic construct
AKDKGIGFNWNYEGFDY

SEQ ID NO: 200 - protein, 7 aa, synthetic construct
DRWDFDY

SEQ ID NO: 201 - protein, 7 aa, synthetic construct
DRWDFDY

SEQ ID NO: 202 - protein, 7 aa, synthetic construct
DRWDFDY

SEQ ID NO: 203 - protein, 8 aa, synthetic construct
ARDRWDFD

SEQ ID NO: 204 - protein, 9 aa, synthetic construct
ARDRWDFDY

SEQ ID NO: 205 - protein, 7 aa, synthetic construct
DRWDFDY

SEQ ID NO: 206 - protein, 7 aa, synthetic construct
DRWDFDY

SEQ ID NO: 207 - protein, 7 aa, synthetic construct
DRWDFDY

SEQ ID NO: 208 - protein, 8 aa, synthetic construct
ARDRWDFD

SEQ ID NO: 209 - protein, 9 aa, synthetic construct
ARDRWDFDY

SEQ ID NO: 210 - protein, 13 aa, synthetic construct
NGVYYGSGAYVDY

SEQ ID NO: 211 - protein, 13 aa, synthetic construct
NGVYYGSGAYVDY

SEQ ID NO: 212 - protein, 13 aa, synthetic construct
NGVYYGSGAYVDY

SEQ ID NO: 213 - protein, 14 aa, synthetic construct
ARNGVYYGSGAYVD

SEQ ID NO: 214 - protein, 15 aa, synthetic construct
ARNGVYYGSGAYVDY

SEQ ID NO: 215 - protein, 13 aa, synthetic construct
NGVYYGSGAYVDY

SEQ ID NO: 216 - protein, 13 aa, synthetic construct
NGVYYGSGAYVDY

SEQ ID NO: 217 - protein, 13 aa, synthetic construct
NGVYYGSGAYVDY

SEQ ID NO: 218 - protein, 14 aa, synthetic construct
ARNGVYYGSGAYVD

SEQ ID NO: 219 - protein, 15 aa, synthetic construct
ARNGVYYGSGAYVDY

SEQ ID NO: 220 - protein, 7 aa, synthetic construct
GFTFSIY

SEQ ID NO: 221 - protein, 10 aa, synthetic construct
GFTFSIYGMH

SEQ ID NO: 222 - protein, 5 aa, synthetic construct
IYGMH

SEQ ID NO: 223 - protein, 6 aa, synthetic construct
SIYGMH

SEQ ID NO: 224 - protein, 8 aa, synthetic construct
GFTFSIYG

SEQ ID NO: 225 - protein, 7 aa, synthetic construct
GFAFNIY

SEQ ID NO: 226 - protein, 10 aa, synthetic construct
GFAFNIYGMH

SEQ ID NO: 227 - protein, 5 aa, synthetic construct
IYGMH

SEQ ID NO: 228 - protein, 6 aa, synthetic construct
NIYGMH

SEQ ID NO: 229 - protein, 8 aa, synthetic construct
GFAFNIYG

SEQ ID NO: 230 - protein, 9 aa, synthetic construct
GFSLSTSGV

SEQ ID NO: 231 - protein, 12 aa, synthetic construct
GFSLSTSGVGVG

SEQ ID NO: 232 - protein, 7 aa, synthetic construct
TSGVGVG

SEQ ID NO: 233 - protein, 8 aa, synthetic construct
STSGVGVG

SEQ ID NO: 234 - protein, 10 aa, synthetic construct
GFSLSTSGVG

SEQ ID NO: 235 - protein, 6 aa, synthetic construct
WYDGSN

SEQ ID NO: 236 - protein, 10 aa, synthetic construct
VIWYDGSNKY

SEQ ID NO: 237 - protein, 17 aa, synthetic construct
VIWYDGSNKYYVDSVKG

SEQ ID NO: 238 - protein, 13 aa, synthetic construct
WVAVIWYDGSNKY

SEQ ID NO: 239 - protein, 8 aa, synthetic construct
IWYDGSNK

SEQ ID NO: 240 - protein, 6 aa, synthetic construct
SYDGSN

SEQ ID NO: 241 - protein, 10 aa, synthetic construct
VISYDGSNKV

SEQ ID NO: 242 - protein, 17 aa, synthetic construct
VISYDGSNKVYADSVKG

SEQ ID NO: 243 - protein, 13 aa, synthetic construct
WVAVISYDGSNKV

SEQ ID NO: 244 - protein, 8 aa, synthetic construct
ISYDGSNK

SEQ ID NO: 245 - protein, 5 aa, synthetic construct
YWNDD

SEQ ID NO: 246 - protein, 9 aa, synthetic construct
LIYWNDDKR

SEQ ID NO: 247 - protein, 16 aa, synthetic construct
LIYWNDDKRYSPSLKS

SEQ ID NO: 248 - protein, 12 aa, synthetic construct
WLALIYWNDDKR

SEQ ID NO: 249 - protein, 7 aa, synthetic construct
IYWNDDK

SEQ ID NO: 250 - protein, 7 aa, synthetic construct
EGNLFDY

SEQ ID NO: 251 - protein, 7 aa, synthetic construct
EGNLFDY

SEQ ID NO: 252 - protein, 7 aa, synthetic construct
EGNLFDY

SEQ ID NO: 253 - protein, 8 aa, synthetic construct
AREGNLFD

SEQ ID NO: 254 - protein, 9 aa, synthetic construct
AREGNLFDY

SEQ ID NO: 255 - protein, 13 aa, synthetic construct
SGGITMVRGVFDY

SEQ ID NO: 256 - protein, 13 aa, synthetic construct
SGGITMVRGVFDY

SEQ ID NO: 257 - protein, 13 aa, synthetic construct
SGGITMVRGVFDY

SEQ ID NO: 258 - protein, 14 aa, synthetic construct
AKSGGITMVRGVFD

SEQ ID NO: 259 - protein, 15 aa, synthetic construct
AKSGGITMVRGVFDY

SEQ ID NO: 260 - protein, 15 aa, synthetic construct
RRTTTVTIYYYYMDV

SEQ ID NO: 261 - protein, 15 aa, synthetic construct
RRTTTVTIYYYYMDV

SEQ ID NO: 262 - protein, 15 aa, synthetic construct
RRTTTVTIYYYYMDV

SEQ ID NO: 263 - protein, 16 aa, synthetic construct
AHRRTTTVTIYYYYMD

SEQ ID NO: 264 - protein, 17 aa, synthetic construct
AHRRTTTVTIYYYYMDV

SEQ ID NO: 265-299 - skipped (35 entries)

SEQ ID NO: 300 - protein, 11 aa, synthetic construct
RASQGIRNNLG

SEQ ID NO: 301 - protein, 11 aa, synthetic construct
RASQGIRNNLG

SEQ ID NO: 302 - protein, 11 aa, synthetic construct
RASQGIRNNLG

SEQ ID NO: 303 - protein, 7 aa, synthetic construct
RNNLGWY

SEQ ID NO: 304 - protein, 6 aa, synthetic construct
QGIRNN

SEQ ID NO: 305 - protein, 11 aa, synthetic construct
RASQGIRNNLG

SEQ ID NO: 306 - protein, 11 aa, synthetic construct
RASQGIRNNLG

SEQ ID NO: 307 - protein, 11 aa, synthetic construct
RASQGIRNNLG

SEQ ID NO: 308 - protein, 7 aa, synthetic construct
RNNLGWY

SEQ ID NO: 309 - protein, 6 aa, synthetic construct
QGIRNN

SEQ ID NO: 310 - protein, 11 aa, synthetic construct
RASQGISNHLA

SEQ ID NO: 311 - protein, 11 aa, synthetic construct
RASQGISNHLA

SEQ ID NO: 312 - protein, 11 aa, synthetic construct
RASQGISNHLA

SEQ ID NO: 313 - protein, 7 aa, synthetic construct
SNHLAWF

SEQ ID NO: 314 - protein, 6 aa, synthetic construct
QGISNH

SEQ ID NO: 315 - protein, 11 aa, synthetic construct
RASQGISNHLA

SEQ ID NO: 316 - protein, 11 aa, synthetic construct
RASQGISNHLA

SEQ ID NO: 317 - protein, 11 aa, synthetic construct
RASQGISNHLA

SEQ ID NO: 318 - protein, 7 aa, synthetic construct
SNHLAWF

SEQ ID NO: 319 - protein, 6 aa, synthetic construct
QGISNH

SEQ ID NO: 320 - protein, 16 aa, synthetic construct
RSSQSLLHSNGYNYLD

SEQ ID NO: 321 - protein, 16 aa, synthetic construct
RSSQSLLHSNGYNYLD

SEQ ID NO: 322 - protein, 16 aa, synthetic construct
RSSQSLLHSNGYNYLD

SEQ ID NO: 323 - protein, 12 aa, synthetic construct
LHSNGYNYLDWY

SEQ ID NO: 324 - protein, 11 aa, synthetic construct
QSLLHSNGYNY

SEQ ID NO: 325 - protein, 16 aa, synthetic construct
RSSQSLLHSNGYNYLD

SEQ ID NO: 326 - protein, 16 aa, synthetic construct
RSSQSLLHSNGYNYLD

SEQ ID NO: 327 - protein, 16 aa, synthetic construct
RSSQSLLHSNGYNYLD

SEQ ID NO: 328 - protein, 12 aa, synthetic construct
LHSNGYNYLDWY

SEQ ID NO: 329 - protein, 11 aa, synthetic construct
QSLLHSNGYNY

SEQ ID NO: 330 - protein, 16 aa, synthetic construct
RSSQSLLHSNGYNYLD

SEQ ID NO: 331 - protein, 16 aa, synthetic construct
RSSQSLLHSNGYNYLD

SEQ ID NO: 332 - protein, 16 aa, synthetic construct
RSSQSLLHSNGYNYLD

SEQ ID NO: 333 - protein, 12 aa, synthetic construct
LHSNGYNYLDWY

SEQ ID NO: 334 - protein, 11 aa, synthetic construct
QSLLHSNGYNY

SEQ ID NO: 335 - protein, 16 aa, synthetic construct
RSSQSLLHSNGYNYLD

SEQ ID NO: 336 - protein, 16 aa, synthetic construct
RSSQSLLHSNGYNYLD

SEQ ID NO: 337 - protein, 16 aa, synthetic construct
RSSQSLLHSNGYNYLD

SEQ ID NO: 338 - protein, 12 aa, synthetic construct
LHSNGYNYLDWY

SEQ ID NO: 339 - protein, 11 aa, synthetic construct
QSLLHSNGYNY

SEQ ID NO: 340 - protein, 7 aa, synthetic construct
AESSLQS

SEQ ID NO: 341 - protein, 7 aa, synthetic construct
AESSLQS

SEQ ID NO: 342 - protein, 7 aa, synthetic construct
AESSLQS

SEQ ID NO: 343 - protein, 10 aa, synthetic construct
LLIYAESSLQ

SEQ ID NO: 344 - skipped

SEQ ID NO: 345 - protein, 7 aa, synthetic construct
AESSLQS

SEQ ID NO: 346 - protein, 7 aa, synthetic construct
AESSLQS

SEQ ID NO: 347 - protein, 7 aa, synthetic construct
AESSLQS

SEQ ID NO: 348 - protein, 10 aa, synthetic construct
LLIYAESSLQ

SEQ ID NO: 349 - skipped

SEQ ID NO: 350 - protein, 7 aa, synthetic construct
AASSLQS

SEQ ID NO: 351 - protein, 7 aa, synthetic construct
AASSLQS

SEQ ID NO: 352 - protein, 7 aa, synthetic construct
AASSLQS

SEQ ID NO: 353 - protein, 10 aa, synthetic construct
SLIYAASSLQ

SEQ ID NO: 354 - skipped

SEQ ID NO: 355 - protein, 7 aa, synthetic construct
AASSLQS

SEQ ID NO: 356 - protein, 7 aa, synthetic construct
AASSLQS

SEQ ID NO: 357 - protein, 7 aa, synthetic construct
AASSLQS

SEQ ID NO: 358 - protein, 10 aa, synthetic construct
SLIYAASSLQ

SEQ ID NO: 359 - skipped

SEQ ID NO: 360 - protein, 7 aa, synthetic construct
LGSNRAS

SEQ ID NO: 361 - protein, 7 aa, synthetic construct
LGSNRAS

SEQ ID NO: 362 - protein, 7 aa, synthetic construct
LGSNRAS

SEQ ID NO: 363 - protein, 10 aa, synthetic construct
LLIYLGSNRA

SEQ ID NO: 364 - skipped

SEQ ID NO: 365 - protein, 7 aa, synthetic construct
LGSNRAS

SEQ ID NO: 366 - protein, 7 aa, synthetic construct
LGSNRAS

SEQ ID NO: 367 - protein, 7 aa, synthetic construct
LGSNRAS

SEQ ID NO: 368 - protein, 10 aa, synthetic construct
LLIYLGSNRA

SEQ ID NO: 369 - skipped

SEQ ID NO: 370 - protein, 7 aa, synthetic construct
LGSNRAS

SEQ ID NO: 371 - protein, 7 aa, synthetic construct
LGSNRAS

SEQ ID NO: 372 - protein, 7 aa, synthetic construct
LGSNRAS

SEQ ID NO: 373 - protein, 10 aa, synthetic construct
LLIYLGSNRA

SEQ ID NO: 374 - skipped

SEQ ID NO: 375 - protein, 7 aa, synthetic construct
LGSNRAS

SEQ ID NO: 376 - protein, 7 aa, synthetic construct
LGSNRAS

SEQ ID NO: 377 - protein, 7 aa, synthetic construct
LGSNRAS

SEQ ID NO: 378 - protein, 10 aa, synthetic construct
LLIYLGSNRA

SEQ ID NO: 379 - skipped

SEQ ID NO: 380 - protein, 9 aa, synthetic construct
LQDFNYPYT

SEQ ID NO: 381 - protein, 9 aa, synthetic construct
LQDFNYPYT

SEQ ID NO: 382 - protein, 9 aa, synthetic construct
LQDFNYPYT

SEQ ID NO: 383 - protein, 8 aa, synthetic construct
LQDFNYPY

SEQ ID NO: 384 - protein, 9 aa, synthetic construct
LQDFNYPYT

SEQ ID NO: 385 - protein, 9 aa, synthetic construct
LQDFNYPYT

SEQ ID NO: 386 - protein, 9 aa, synthetic construct
LQDFNYPYT

SEQ ID NO: 387 - protein, 9 aa, synthetic construct
LQDFNYPYT

SEQ ID NO: 388 - protein, 8 aa, synthetic construct
LQDFNYPY

SEQ ID NO: 389 - protein, 9 aa, synthetic construct
LQDFNYPYT

SEQ ID NO: 390 - protein, 9 aa, synthetic construct
QQYVTYPLT

SEQ ID NO: 391 - protein, 9 aa, synthetic construct
QQYVTYPLT

SEQ ID NO: 392 - protein, 9 aa, synthetic construct
QQYVTYPLT

SEQ ID NO: 393 - protein, 8 aa, synthetic construct
QQYVTYPL

SEQ ID NO: 394 - protein, 9 aa, synthetic construct
QQYVTYPLT

SEQ ID NO: 395 - protein, 9 aa, synthetic construct
QQYVTYPLT

SEQ ID NO: 396 - protein, 9 aa, synthetic construct
QQYVTYPLT

SEQ ID NO: 397 - protein, 9 aa, synthetic construct
QQYVTYPLT

SEQ ID NO: 398 - protein, 8 aa, synthetic construct
QQYVTYPL

SEQ ID NO: 399 - protein, 9 aa, synthetic construct
QQYVTYPLT

SEQ ID NO: 400 - protein, 9 aa, synthetic construct
MQALQTPRT

SEQ ID NO: 401 - protein, 9 aa, synthetic construct
MQALQTPRT

SEQ ID NO: 402 - protein, 9 aa, synthetic construct
MQALQTPRT

SEQ ID NO: 403 - protein, 8 aa, synthetic construct
MQALQTPR

SEQ ID NO: 404 - protein, 9 aa, synthetic construct
MQALQTPRT

SEQ ID NO: 405 - protein, 9 aa, synthetic construct
MQALQTPRT

SEQ ID NO: 406 - protein, 9 aa, synthetic construct
MQALQTPRT

SEQ ID NO: 407 - protein, 9 aa, synthetic construct
MQALQTPRT

SEQ ID NO: 408 - protein, 8 aa, synthetic construct
MQALQTPR

SEQ ID NO: 409 - protein, 9 aa, synthetic construct
MQALQTPRT

SEQ ID NO: 410 - protein, 9 aa, synthetic construct
MQALQTPRT

SEQ ID NO: 411 - protein, 9 aa, synthetic construct
MQALQTPRT

SEQ ID NO: 412 - protein, 9 aa, synthetic construct
MQALQTPRT

SEQ ID NO: 413 - protein, 8 aa, synthetic construct
MQALQTPR

SEQ ID NO: 414 - protein, 9 aa, synthetic construct
MQALQTPRT

SEQ ID NO: 415 - protein, 9 aa, synthetic construct
MQALQTPRT

SEQ ID NO: 416 - protein, 9 aa, synthetic construct
MQALQTPRT

SEQ ID NO: 417 - protein, 9 aa, synthetic construct
MQALQTPRT

SEQ ID NO: 418 - protein, 8 aa, synthetic construct
MQALQTPR

SEQ ID NO: 419 - protein, 9 aa, synthetic construct
MQALQTPRT

SEQ ID NO: 420 - protein, 11 aa, synthetic construct
RASQSISSWLA

SEQ ID NO: 421 - protein, 11 aa, synthetic construct
RASQSISSWLA

SEQ ID NO: 422 - protein, 11 aa, synthetic construct
RASQSISSWLA

SEQ ID NO: 423 - protein, 7 aa, synthetic construct
SSWLAWY

SEQ ID NO: 424 - protein, 6 aa, synthetic construct
QSISSW

SEQ ID NO: 425 - protein, 11 aa, synthetic construct
RASQGIRNDLG

SEQ ID NO: 426 - protein, 11 aa, synthetic construct
RASQGIRNDLG

SEQ ID NO: 427 - protein, 11 aa, synthetic construct
RASQGIRNDLG

SEQ ID NO: 428 - protein, 7 aa, synthetic construct
RNDLGWY

SEQ ID NO: 429 - protein, 6 aa, synthetic construct
QGIRND

SEQ ID NO: 430 - protein, 11 aa, synthetic construct
RASQSVSSNLA

SEQ ID NO: 431 - protein, 11 aa, synthetic construct
RASQSVSSNLA

SEQ ID NO: 432 - protein, 11 aa, synthetic construct
RASQSVSSNLA

SEQ ID NO: 433 - protein, 7 aa, synthetic construct
SSNLAWY

SEQ ID NO: 434 - protein, 6 aa, synthetic construct
QSVSSN

SEQ ID NO: 435 - protein, 7 aa, synthetic construct
KASSLES

SEQ ID NO: 436 - protein, 7 aa, synthetic construct
KASSLES

SEQ ID NO: 437 - protein, 7 aa, synthetic construct
KASSLES

SEQ ID NO: 438 - protein, 10 aa, synthetic construct
LLIYKASSLE

SEQ ID NO: 439 - skipped

SEQ ID NO: 440 - protein, 7 aa, synthetic construct
AASSLHS

SEQ ID NO: 441 - protein, 7 aa, synthetic construct
AASSLHS

SEQ ID NO: 442 - protein, 7 aa, synthetic construct
AASSLHS

SEQ ID NO: 443 - protein, 10 aa, synthetic construct
RLIHAASSLH

SEQ ID NO: 444 - skipped

SEQ ID NO: 445 - protein, 7 aa, synthetic construct
GASTRAT

SEQ ID NO: 446 - protein, 7 aa, synthetic construct
GASTRAT

SEQ ID NO: 447 - protein, 7 aa, synthetic construct
GASTRAT

SEQ ID NO: 448 - protein, 10 aa, synthetic construct
LLIYGASTRA

SEQ ID NO: 449 - skipped

SEQ ID NO: 450 - protein, 8 aa, synthetic construct
QQYKSYYT

SEQ ID NO: 451 - protein, 8 aa, synthetic construct
QQYKSYYT

SEQ ID NO: 452 - protein, 8 aa, synthetic construct
QQYKSYYT

SEQ ID NO: 453 - protein, 7 aa, synthetic construct
QQYKSYY

SEQ ID NO: 454 - protein, 8 aa, synthetic construct
QQYKSYYT

SEQ ID NO: 455 - protein, 9 aa, synthetic construct
LQHNRYPIT

SEQ ID NO: 456 - protein, 9 aa, synthetic construct
LQHNRYPIT

SEQ ID NO: 457 - protein, 9 aa, synthetic construct
LQHNRYPIT

SEQ ID NO: 458 - protein, 8 aa, synthetic construct
LQHNRYPI

SEQ ID NO: 459 - protein, 9 aa, synthetic construct
LQHNRYPIT

SEQ ID NO: 460 - protein, 9 aa, synthetic construct
QQYNNWPFT

SEQ ID NO: 461 - protein, 9 aa, synthetic construct
QQYNNWPFT

SEQ ID NO: 462 - protein, 9 aa, synthetic construct
QQYNNWPFT

SEQ ID NO: 463 - protein, 8 aa, synthetic construct
QQYNNWPF

SEQ ID NO: 464 - protein, 9 aa, synthetic construct
QQYNNWPFT

SEQ ID NO: 465 - protein, 7 aa, synthetic construct
GFMFSSY

SEQ ID NO: 466 - protein, 10 aa, synthetic construct
GFMFSSYSMN

SEQ ID NO: 467 - protein, 5 aa, synthetic construct
SYSMN

SEQ ID NO: 468 - protein, 6 aa, synthetic construct
SSYSMN

SEQ ID NO: 469 - protein, 8 aa, synthetic construct
GFMFSSYS

SEQ ID NO: 470 - protein, 7 aa, synthetic construct
GFTFSNY

SEQ ID NO: 471 - protein, 10 aa, synthetic construct
GFTFSNYAMT

SEQ ID NO: 472 - protein, 5 aa, synthetic construct
NYAMT

SEQ ID NO: 473 - protein, 6 aa, synthetic construct
SNYAMT

SEQ ID NO: 474 - protein, 8 aa, synthetic construct
GFTFSNYA

SEQ ID NO: 475 - protein, 7 aa, synthetic construct
GFTFSNY

SEQ ID NO: 476 - protein, 10 aa, synthetic construct
GFTFSNYAMT

SEQ ID NO: 477 - protein, 5 aa, synthetic construct
NYAMT

SEQ ID NO: 478 - protein, 6 aa, synthetic construct
SNYAMT

SEQ ID NO: 479 - protein, 8 aa, synthetic construct
GFTFSNYA

SEQ ID NO: 480 - protein, 7 aa, synthetic construct
GFTFSSY

SEQ ID NO: 481 - protein, 10 aa, synthetic construct
GFTFSSYDMH

SEQ ID NO: 482 - protein, 5 aa, synthetic construct
SYDMH

SEQ ID NO: 483 - protein, 6 aa, synthetic construct
SSYDMH

SEQ ID NO: 484 - protein, 8 aa, synthetic construct
GFTFSSYD

SEQ ID NO: 485 - protein, 7 aa, synthetic construct
GITFSNA

SEQ ID NO: 486 - protein, 10 aa, synthetic construct
GITFSNAWMS

SEQ ID NO: 487 - protein, 5 aa, synthetic construct
NAWMS

SEQ ID NO: 488 - protein, 6 aa, synthetic construct
SNAWMS

SEQ ID NO: 489 - protein, 8 aa, synthetic construct
GITFSNAW

SEQ ID NO: 490 - protein, 6 aa, synthetic construct
SSSSST

SEQ ID NO: 491 - protein, 10 aa, synthetic construct
YISSSSSTIY

SEQ ID NO: 492 - protein, 17 aa, synthetic construct
YISSSSSTIYDADSVKG

SEQ ID NO: 493 - protein, 13 aa, synthetic construct
WVSYISSSSSTIY

SEQ ID NO: 494 - protein, 8 aa, synthetic construct
ISSSSSTI

SEQ ID NO: 495 - protein, 6 aa, synthetic construct
SGFGGS

SEQ ID NO: 496 - protein, 10 aa, synthetic construct
AISGFGGSTY

SEQ ID NO: 497 - protein, 17 aa, synthetic construct
AISGFGGSTYYADSVKG

SEQ ID NO: 498 - protein, 13 aa, synthetic construct
WVSAISGFGGSTY

SEQ ID NO: 499 - protein, 8 aa, synthetic construct
ISGFGGST

SEQ ID NO: 500 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 501 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 502 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 503 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 504 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 505 - protein, 330 aa, synthetic construct; identical to SEQ ID NO: 504

SEQ ID NO: 506 - protein, 330 aa, synthetic construct; identical to SEQ ID NO: 504

SEQ ID NO: 507 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEEFGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 508 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 509 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 510 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 511 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 512 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 513 - protein, 6 aa, synthetic construct
SGYGGS

SEQ ID NO: 514 - protein, 10 aa, synthetic construct
AISGYGGSTY

SEQ ID NO: 515 - protein, 17 aa, synthetic construct
AISGYGGSTYYAASVKG

SEQ ID NO: 516 - protein, 13 aa, synthetic construct
WVSAISGYGGSTY

SEQ ID NO: 517 - protein, 8 aa, synthetic construct
ISGYGGST

SEQ ID NO: 518 - protein, 5 aa, synthetic construct
GIGGD

SEQ ID NO: 519 - protein, 9 aa, synthetic construct
SIGIGGDTY

SEQ ID NO: 520 - protein, 16 aa, synthetic construct
SIGIGGDTYYPGSVKG

SEQ ID NO: 521 - protein, 12 aa, synthetic construct
WVSSIGIGGDTY

SEQ ID NO: 522 - protein, 7 aa, synthetic construct
IGIGGDT

SEQ ID NO: 523 - protein, 8 aa, synthetic construct
KSKTYGGT

SEQ ID NO: 524 - protein, 12 aa, synthetic construct
RIKSKTYGGTTD

SEQ ID NO: 525 - protein, 19 aa, synthetic construct
RIKSKTYGGTTDYAAPVKG

SEQ ID NO: 526 - protein, 15 aa, synthetic construct
WVGRIKSKTYGGTTD

SEQ ID NO: 527 - protein, 10 aa, synthetic construct
IKSKTYGGTT

SEQ ID NO: 528 - protein, 13 aa, synthetic construct
EAYPGYYYNYMDV

SEQ ID NO: 529 - protein, 13 aa, synthetic construct
EAYPGYYYNYMDV

SEQ ID NO: 530 - protein, 13 aa, synthetic construct
EAYPGYYYNYMDV

SEQ ID NO: 531 - protein, 14 aa, synthetic construct
TREAYPGYYYNYMD

SEQ ID NO: 532 - protein, 15 aa, synthetic construct
TREAYPGYYYNYMDV

SEQ ID NO: 533 - protein, 9 aa, synthetic construct
DHDYYAFDY

SEQ ID NO: 534 - protein, 9 aa, synthetic construct
DHDYYAFDY

SEQ ID NO: 535 - protein, 9 aa, synthetic construct
DHDYYAFDY

SEQ ID NO: 536 - protein, 10 aa, synthetic construct
AKDHDYYAFD

SEQ ID NO: 537 - protein, 11 aa, synthetic construct
AKDHDYYAFDY

SEQ ID NO: 538 - protein, 9 aa, synthetic construct
DHDYYAFDY

SEQ ID NO: 539 - protein, 9 aa, synthetic construct
DHDYYAFDY

SEQ ID NO: 540 - protein, 9 aa, synthetic construct
DHDYYAFDY

SEQ ID NO: 541 - protein, 10 aa, synthetic construct
AKDHDYYAFD

SEQ ID NO: 542 - protein, 11 aa, synthetic construct
AKDHDYYAFDY

SEQ ID NO: 543 - protein, 12 aa, synthetic construct
GEWDLLWYFMDV

SEQ ID NO: 544 - protein, 12 aa, synthetic construct
GEWDLLWYFMDV

SEQ ID NO: 545 - protein, 12 aa, synthetic construct
GEWDLLWYFMDV

SEQ ID NO: 546 - protein, 13 aa, synthetic construct
ARGEWDLLWYFMD

SEQ ID NO: 547 - protein, 14 aa, synthetic construct
ARGEWDLLWYFMDV

SEQ ID NO: 548 - protein, 14 aa, synthetic construct
DPWNYVNYNYFMDV

SEQ ID NO: 549 - protein, 14 aa, synthetic construct
DPWNYVNYNYFMDV

SEQ ID NO: 550 - protein, 14 aa, synthetic construct
DPWNYVNYNYFMDV

SEQ ID NO: 551 - protein, 15 aa, synthetic construct
TTDPWNYVNYNYFMD

SEQ ID NO: 552 - protein, 16 aa, synthetic construct
TTDPWNYVNYNYFMDV

SEQ ID NO: 553 - protein, 11 aa, synthetic construct
RASQSISSRLA

SEQ ID NO: 554 - protein, 11 aa, synthetic construct
RASQSISSRLA

SEQ ID NO: 555 - protein, 11 aa, synthetic construct
RASQSISSRLA

SEQ ID NO: 556 - protein, 7 aa, synthetic construct
SSRLAWY

SEQ ID NO: 557 - protein, 6 aa, synthetic construct
QSISSR

SEQ ID NO: 558 - protein, 11 aa, synthetic construct
RASQSISSYLN

SEQ ID NO: 559 - protein, 11 aa, synthetic construct
RASQSISSYLN

SEQ ID NO: 560 - protein, 11 aa, synthetic construct
RASQSISSYLN

SEQ ID NO: 561 - protein, 7 aa, synthetic construct
SSYLNWY

SEQ ID NO: 562 - protein, 6 aa, synthetic construct
QSISSY

SEQ ID NO: 563 - protein, 11 aa, synthetic construct
RASQSINSYLN

SEQ ID NO: 564 - protein, 11 aa, synthetic construct
RASQSINSYLN

SEQ ID NO: 565 - protein, 11 aa, synthetic construct
RASQSINSYLN

SEQ ID NO: 566 - protein, 7 aa, synthetic construct
NSYLNWY

SEQ ID NO: 567 - protein, 6 aa, synthetic construct
QSINSY

SEQ ID NO: 568 - protein, 11 aa, synthetic construct
RASQGISSWLA

SEQ ID NO: 569 - protein, 11 aa, synthetic construct
RASQGISSWLA

SEQ ID NO: 570 - protein, 11 aa, synthetic construct
RASQGISSWLA

SEQ ID NO: 571 - protein, 7 aa, synthetic construct
SSWLAWY

SEQ ID NO: 572 - protein, 6 aa, synthetic construct
QGISSW

SEQ ID NO: 573 - protein, 11 aa, synthetic construct
RASQDIRNYLA

SEQ ID NO: 574 - protein, 11 aa, synthetic construct
RASQDIRNYLA

SEQ ID NO: 575 - protein, 11 aa, synthetic construct
RASQDIRNYLA

SEQ ID NO: 576 - protein, 7 aa, synthetic construct
RNYLAWY

SEQ ID NO: 577 - protein, 6 aa, synthetic construct
QDIRNY

SEQ ID NO: 578 - protein, 7 aa, synthetic construct
KASNLES

SEQ ID NO: 579 - protein, 7 aa, synthetic construct
KASNLES

SEQ ID NO: 580 - protein, 7 aa, synthetic construct
KASNLES

SEQ ID NO: 581 - protein, 10 aa, synthetic construct
LLIYKASNLE

SEQ ID NO: 582 - skipped

SEQ ID NO: 583 - protein, 7 aa, synthetic construct
AVSSLQS

SEQ ID NO: 584 - protein, 7 aa, synthetic construct
AVSSLQS

SEQ ID NO: 585 - protein, 7 aa, synthetic construct
AVSSLQS

SEQ ID NO: 586 - protein, 10 aa, synthetic construct
LLIYAVSSLQ

SEQ ID NO: 587 - skipped

SEQ ID NO: 588 - protein, 7 aa, synthetic construct
SASSLQS

SEQ ID NO: 589 - protein, 7 aa, synthetic construct
SASSLQS

SEQ ID NO: 590 - protein, 7 aa, synthetic construct
SASSLQS

SEQ ID NO: 591 - protein, 10 aa, synthetic construct
LLIYSASSLQ

SEQ ID NO: 592 - skipped

SEQ ID NO: 593 - protein, 7 aa, synthetic construct
AASSLQS

SEQ ID NO: 594 - protein, 7 aa, synthetic construct
AASSLQS

SEQ ID NO: 595 - protein, 7 aa, synthetic construct
AASSLQS

SEQ ID NO: 596 - protein, 10 aa, synthetic construct
LLIYAASSLQ

SEQ ID NO: 597 - skipped

SEQ ID NO: 598 - protein, 7 aa, synthetic construct
AASTLQS

SEQ ID NO: 599 - protein, 7 aa, synthetic construct
AASTLQS

SEQ ID NO: 600 - protein, 7 aa, synthetic construct
AASTLQS

SEQ ID NO: 601 - protein, 10 aa, synthetic construct
LLVYAASTLQ

SEQ ID NO: 602 - skipped

SEQ ID NO: 603 - protein, 9 aa, synthetic construct
QQYNSYSRT

SEQ ID NO: 604 - protein, 9 aa, synthetic construct
QQYNSYSRT

SEQ ID NO: 605 - protein, 9 aa, synthetic construct
QQYNSYSRT

SEQ ID NO: 606 - protein, 8 aa, synthetic construct
QQYNSYSR

SEQ ID NO: 607 - protein, 9 aa, synthetic construct
QQYNSYSRT

SEQ ID NO: 608 - protein, 9 aa, synthetic construct
QQSYSTPYT

SEQ ID NO: 609 - protein, 9 aa, synthetic construct
QQSYSTPYT

SEQ ID NO: 610 - protein, 9 aa, synthetic construct
QQSYSTPYT

SEQ ID NO: 611 - protein, 8 aa, synthetic construct
QQSYSTPY

SEQ ID NO: 612 - protein, 9 aa, synthetic construct
QQSYSTPYT

SEQ ID NO: 613 - protein, 9 aa, synthetic construct
QQSYSTPYT

SEQ ID NO: 614 - protein, 9 aa, synthetic construct
QQSYSTPYT

SEQ ID NO: 615 - protein, 9 aa, synthetic construct
QQSYSTPYT

SEQ ID NO: 616 - protein, 8 aa, synthetic construct
QQSYSTPY

SEQ ID NO: 617 - protein, 9 aa, synthetic construct
QQSYSTPYT

SEQ ID NO: 618 - protein, 9 aa, synthetic construct
QQANSFPLT

SEQ ID NO: 619 - protein, 9 aa, synthetic construct
QQANSFPLT

SEQ ID NO: 620 - protein, 9 aa, synthetic construct
QQANSFPLT

SEQ ID NO: 621 - protein, 8 aa, synthetic construct
QQANSFPL

SEQ ID NO: 622 - protein, 9 aa, synthetic construct
QQANSFPLT

SEQ ID NO: 623 - protein, 9 aa, synthetic construct
QNYFSVPLT

SEQ ID NO: 624 - protein, 9 aa, synthetic construct
QNYFSVPLT

SEQ ID NO: 625 - protein, 9 aa, synthetic construct
QNYFSVPLT

SEQ ID NO: 626 - protein, 8 aa, synthetic construct
QNYFSVPL

SEQ ID NO: 627 - protein, 9 aa, synthetic construct
QNYFSVPLT

SEQ ID NO: 628 - protein, 10 aa, synthetic construct
VSGGSISGYY

SEQ ID NO: 629 - protein, 10 aa, synthetic construct
ASGFNFDDYA

SEQ ID NO: 630 - protein, 10 aa, synthetic construct
VSGFTFSSYD

SEQ ID NO: 631 - protein, 10 aa, synthetic construct
ASGFMFSSYS

SEQ ID NO: 632 - protein, 10 aa, synthetic construct
ASGFTFSNYA

SEQ ID NO: 633 - protein, 10 aa, synthetic construct
ASGFTFSNYA

SEQ ID NO: 634 - protein, 10 aa, synthetic construct
VSGITFSNAW

SEQ ID NO: 635 - protein, 5 aa, synthetic construct
GYYWS

SEQ ID NO: 636 - protein, 5 aa, synthetic construct
DYAMH

SEQ ID NO: 637 - protein, 5 aa, synthetic construct
SYDMH

SEQ ID NO: 638 - protein, 5 aa, synthetic construct
SYSMN

SEQ ID NO: 639 - protein, 5 aa, synthetic construct
NYAMT

SEQ ID NO: 640 - protein, 5 aa, synthetic construct
NYAMT

SEQ ID NO: 641 - protein, 5 aa, synthetic construct
NAWMS

SEQ ID NO: 642 - protein, 16 aa, synthetic construct
IFYSGYTKYNPSLKSR

SEQ ID NO: 643 - protein, 17 aa, synthetic construct
ISWRSGNIGYADSVKGR

SEQ ID NO: 644 - protein, 16 aa, synthetic construct
IGIGGDTYYPGSVKGR

SEQ ID NO: 645 - protein, 17 aa, synthetic construct
ISSSSSTIYDADSVKGR

SEQ ID NO: 646 - protein, 17 aa, synthetic construct
ISGFGGSTYYADSVKGR

SEQ ID NO: 647 - protein, 17 aa, synthetic construct
ISGYGGSTYYAASVKGR

SEQ ID NO: 648 - protein, 19 aa, synthetic construct
IKSKTYGGTTDYAAPVKGR

SEQ ID NO: 649 - protein, 12 aa, synthetic construct
WIGYIFYSGYTK

SEQ ID NO: 650 - protein, 13 aa, synthetic construct
WVSGISWRSGNIG

SEQ ID NO: 651 - protein, 12 aa, synthetic construct
WVSSIGIGGDTY

SEQ ID NO: 652 - protein, 13 aa, synthetic construct
WVSYISSSSSTIY

SEQ ID NO: 653 - protein, 13 aa, synthetic construct
WVSAISGFGGSTY

SEQ ID NO: 654 - protein, 13 aa, synthetic construct
WVSAISGYGGSTY

SEQ ID NO: 655 - protein, 15 aa, synthetic construct
WVGRIKSKTYGGTTD

SEQ ID NO: 656 - protein, 6 aa, synthetic construct
DRWDFD

SEQ ID NO: 657 - protein, 14 aa, synthetic construct
DKGIGFNWNYEGFD

SEQ ID NO: 658 - protein, 11 aa, synthetic construct
GEWDLLWYFMD

SEQ ID NO: 659 - protein, 12 aa, synthetic construct
EAYPGYYYNYMD

SEQ ID NO: 660 - protein, 8 aa, synthetic construct
DHDYYAFD

SEQ ID NO: 661 - protein, 8 aa, synthetic construct
DHDYYAFD

SEQ ID NO: 662 - protein, 13 aa, synthetic construct
DPWNYVNYNYFMD

SEQ ID NO: 663 - protein, 7 aa, synthetic construct
DRWDFDY

SEQ ID NO: 664 - protein, 15 aa, synthetic construct
DKGIGFNWNYEGFDY

SEQ ID NO: 665 - protein, 12 aa, synthetic construct
GEWDLLWYFMDV

SEQ ID NO: 666 - protein, 13 aa, synthetic construct
EAYPGYYYNYMDV

SEQ ID NO: 667 - protein, 9 aa, synthetic construct
DHDYYAFDY

SEQ ID NO: 668 - protein, 9 aa, synthetic construct
DHDYYAFDY

SEQ ID NO: 669 - protein, 14 aa, synthetic construct
DPWNYVNYNYFMDV

SEQ ID NO: 670 - protein, 8 aa, synthetic construct
ASQGIRNN

SEQ ID NO: 671 - protein, 8 aa, synthetic construct
ASQGISNH

SEQ ID NO: 672 - protein, 8 aa, synthetic construct
ASQGISSW

SEQ ID NO: 673 - protein, 8 aa, synthetic construct
ASQSISSR

SEQ ID NO: 674 - protein, 8 aa, synthetic construct
ASQSISSY

SEQ ID NO: 675 - protein, 8 aa, synthetic construct
ASQSINSY

SEQ ID NO: 676 - protein, 8 aa, synthetic construct
ASQDIRNY

SEQ ID NO: 677 - protein, 11 aa, synthetic construct
RASQGIRNNLG

SEQ ID NO: 678 - protein, 11 aa, synthetic construct
RASQGISNHLA

SEQ ID NO: 679 - protein, 11 aa, synthetic construct
RASQGISSWLA

SEQ ID NO: 680 - protein, 11 aa, synthetic construct
RASQSISSRLA

SEQ ID NO: 681 - protein, 11 aa, synthetic construct
RASQSISSYLN

SEQ ID NO: 682 - protein, 11 aa, synthetic construct
RASQSINSYLN

SEQ ID NO: 683 - protein, 11 aa, synthetic construct
RASQDIRNYLA

SEQ ID NO: 684 - protein, 12 aa, synthetic construct
AESSLQSGVPSR

SEQ ID NO: 685 - protein, 12 aa, synthetic construct
AASSLQSGVPSR

SEQ ID NO: 686 - protein, 12 aa, synthetic construct
AASSLQSGVPSR

SEQ ID NO: 687 - protein, 12 aa, synthetic construct
KASNLESGVPSR

SEQ ID NO: 688 - protein, 12 aa, synthetic construct
AVSSLQSGVPSR

SEQ ID NO: 689 - protein, 12 aa, synthetic construct
SASSLQSGVPSR

SEQ ID NO: 690 - protein, 12 aa, synthetic construct
AASTLQSGVPSR

SEQ ID NO: 691 - protein, 7 aa, synthetic construct
AESSLQS

SEQ ID NO: 692 - protein, 7 aa, synthetic construct
AASSLQS

SEQ ID NO: 693 - protein, 7 aa, synthetic construct
AASSLQS

SEQ ID NO: 694 - protein, 7 aa, synthetic construct
KASNLES

SEQ ID NO: 695 - protein, 7 aa, synthetic construct
AVSSLQS

SEQ ID NO: 696 - protein, 7 aa, synthetic construct
SASSLQS

SEQ ID NO: 697 - protein, 7 aa, synthetic construct
AASTLQS

SEQ ID NO: 698 - protein, 6 aa, synthetic construct
DFNYPY

SEQ ID NO: 699 - protein, 6 aa, synthetic construct
YVTYPL

SEQ ID NO: 700 - protein, 6 aa, synthetic construct
ANSFPL

SEQ ID NO: 701 - protein, 6 aa, synthetic construct
YNSYSR

SEQ ID NO: 702 - protein, 6 aa, synthetic construct
SYSTPY

SEQ ID NO: 703 - protein, 6 aa, synthetic construct
SYSTPY

SEQ ID NO: 704 - protein, 6 aa, synthetic construct
YFSVPL

SEQ ID NO: 705 - protein, 9 aa, synthetic construct
LQDFNYPYT

SEQ ID NO: 706 - protein, 9 aa, synthetic construct
QQYVTYPLT

SEQ ID NO: 707 - protein, 9 aa, synthetic construct
QQANSFPLT

SEQ ID NO: 708 - protein, 9 aa, synthetic construct
QQYNSYSRT

SEQ ID NO: 709 - protein, 9 aa, synthetic construct
QQSYSTPYT

SEQ ID NO: 710 - protein, 9 aa, synthetic construct
QQSYSTPYT

SEQ ID NO: 711 - protein, 9 aa, synthetic construct
QNYFSVPLT

SEQ ID NO: 712-723 - skipped (12 entries)

SEQ ID NO: 724 - protein, 17 aa, synthetic construct
  VARIANT 1: note = X can be Y, G or S
  VARIANT 3: note = X can be S, G or absent
  VARIANT 4: note = X can be F, W or I
  VARIANT 5: note = X can be Y, R or absent
  VARIANT 6: note = X can be G or S
  VARIANT 8: note = X can be Y, N or D
  VARIANT 9: note = X can be T or I
  VARIANT 10: note = X can be K, G or Y
  VARIANT 12: note = X can be N, A or P
  VARIANT 13: note = X can be P, D or G
  VARIANT 15: note = X can be L or V
  VARIANT 17: note = X can be S or G
XIXXXXGXXXYXXSXKX

SEQ ID NO: 725-733 - skipped (9 entries)

SEQ ID NO: 734 - protein, 17 aa, synthetic construct
  VARIANT 2: note = X can be S, G or absent
  VARIANT 3: note = X can be F, W or I
  VARIANT 4: note = X can be Y, R or absent
  VARIANT 5: note = X can be G or S
  VARIANT 7: note = X can be Y, N or D
  VARIANT 8: note = X can be T or I
  VARIANT 9: note = X can be K, G or Y
  VARIANT 11: note = X can be N, A or P
  VARIANT 12: note = X can be P, D or G
  VARIANT 14: note = X can be L or V
  VARIANT 16: note = X can be G or S
IXXXXGXXXYXXSXKXR

SEQ ID NO: 735-737 - skipped (3 entries)

SEQ ID NO: 738 - protein, 17 aa, synthetic construct
  VARIANT 2: note = X can be K or R
  VARIANT 3: note = X can be D or absent
  VARIANT 4: note = X can be R, K or G
  VARIANT 5: note = X can be G, E or absent
  VARIANT 6: note = X can be I, W or absent
  VARIANT 7: note = X can be G, D or absent
  VARIANT 8: note = X can be F, L or absent
  VARIANT 9: note = X can be N, L or absent
  VARIANT 11: note = X can be N, Y or absent
  VARIANT 12: note = X can be Y or absent
  VARIANT 13: note = X can be D, E or absent
  VARIANT 14: note = X can be G or absent
  VARIANT 16: note = X can be M or absent
AXXXXXXXXWXXXXFXD

SEQ ID NO: 739 - skipped

SEQ ID NO: 740 - protein, 18 aa, synthetic construct
  VARIANT 2: note = X can be K or R
  VARIANT 3: note = X can be D or absent
  VARIANT 4: note = X can be R, K or G
  VARIANT 5: note = X can be G, E or absent
  VARIANT 6: note = X can be I, W or absent
  VARIANT 7: note = X can be G, D or absent
  VARIANT 8: note = X can be F, L or absent
  VARIANT 9: note = X can be N, L or absent
  VARIANT 11: note = X can be N, Y or absent
  VARIANT 12: note = X can be Y or absent
  VARIANT 13: note = X can be D, E or absent
  VARIANT 14: note = X can be G or absent
  VARIANT 16: note = X can be M or absent
  VARIANT 18: note = X can be Y or V
AXXXXXXXXWXXXXFXDX

SEQ ID NO: 741-743 - skipped (3 entries)

SEQ ID NO: 744 - protein, 11 aa, synthetic construct
  VARIANT 7: note = X can be R or S
  VARIANT 8: note = X can be N or S
  VARIANT 9: note = X can be N, H or W
  VARIANT 11: note = X can be G or A
RASQGIXXXLX

SEQ ID NO: 745 - protein, 11 aa, synthetic construct
  VARIANT 5: note = X can be G, S or D
  VARIANT 7: note = X can be R, S or N
  VARIANT 8: note = X can be N or S
  VARIANT 9: note = X can be N, H, W, R or Y
  VARIANT 11: note = X can be G, A or N
RASQXIXXXLX

SEQ ID NO: 746-749 - skipped (4 entries)

SEQ ID NO: 750 - protein, 8 aa, synthetic construct
  VARIANT 6: note = X can be R or S
  VARIANT 7: note = X can be N or S
  VARIANT 8: note = X can be N, H or W
ASQGIXXX

SEQ ID NO: 751 - protein, 8 aa, synthetic construct
  VARIANT 4: note = X can be G, S or D
  VARIANT 6: note = X can be R, S or N
  VARIANT 7: note = X can be N or S
  VARIANT 8: note = X can be N, H, W, R or Y
ASQXIXXX

SEQ ID NO: 752 - protein, 7 aa, synthetic construct
  VARIANT 2: note = X can be A or E
AXSSLQS

SEQ ID NO: 753 - skipped

SEQ ID NO: 754 - protein, 10 aa, synthetic construct
  VARIANT 1: note = X can be L or S
  VARIANT 6: note = X can be A or E
XLIYAXSSLQ

SEQ ID NO: 755 - protein, 10 aa, synthetic construct
  VARIANT 1: note = X can be L or S
  VARIANT 3: note = X can be I or V
  VARIANT 5: note = X can be A, K or S
  VARIANT 6: note = X can be A, E or V
  VARIANT 8: note = X can be S, N or T
XLXYXXSXLZ

SEQ ID NO: 756-757 - skipped (2 entries)

SEQ ID NO: 758 - protein, 12 aa, synthetic construct
  VARIANT 2: note = X can be A or E
AXSSLQSGVPSR

SEQ ID NO: 759 - protein, 12 aa, synthetic construct
  VARIANT 1: note = X can be A, K or S
  VARIANT 2: note = X can be A, E or V
  VARIANT 4: note = X can be S, N or T
XXSXLZSGVPSR

SEQ ID NO: 760-765 - skipped (6 entries)

SEQ ID NO: 766 - protein, 124 aa, synthetic construct
QVQLQESGPGLVKPSETLSLTCTVSGGSISSYIWSWIRQPAGKGLEWIGRIYASGNTNYNPSLKSRVTISVDTSKNQFSLKLSSMTAADTAVYYCARDYRVAGTYYYYYGLDVWGQGTTVTVSS

SEQ ID NO: 767 - protein, 110 aa, synthetic construct
QSALTQPASVSGSPGQSITISCTGTSSDVGVYDYVSWYQQHPGKAPKLMIYEVSNRPSGVSNRFSGSKSGNTASLTISGLQTEDEADYYCSSYTSRSTWVFGGGTKLTVL

SEQ ID NO: 768 - protein, 454 aa, synthetic construct
QVQLQESGPGLVKPSETLSLTCTVSGGSISSYIWSWIRQPAGKGLEWIGRIYASGNTNYNPSLKSRVTISVDTSKNQFSLKLSSMTAADTAVYYCARDYRVAGTYYYYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 769 - protein, 216 aa, synthetic construct
QSALTQPASVSGSPGQSITISCTGTSSDVGVYDYVSWYQQHPGKAPKLMIYEVSNRPSGVSNRFSGSKSGNTASLTISGLQTEDEADYYCSSYTSRSTWVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 770 - protein, 120 aa, synthetic construct
EVKLQQSGDELVRPGASVKMSCKASGYTFTDYAMHWVKQSPGQGLEWIGSIIPLIDTTSYNQKFKGKATLTADKSSNTAYMELSRLTSEDSAVYYCARLGSRYLEGFDYWGQGVMVTVSS

SEQ ID NO: 771 - protein, 107 aa, synthetic construct
DIQMTQSPSSMSASLGDTVTINCLASQDIGNYLSWYQQKPGKSPKLMIYGATNLEDGVPSRFSGSGSGSDYSLTINSLGYDDEGIYHCHQYYEYPLTFGSGTKLEIK

SEQ ID NO: 772 - protein, 450 aa, synthetic construct
EVKLQQSGDELVRPGASVKMSCKASGYTFTDYAMHWVKQSPGQGLEWIGSIIPLIDTTSYNQKFKGKATLTADKSSNTAYMELSRLTSEDSAVYYCARLGSRYLEGFDYWGQGVMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 773 - protein, 214 aa, synthetic construct
DIQMTQSPSSMSASLGDTVTINCLASQDIGNYLSWYQQKPGKSPKLMIYGATNLEDGVPSRFSGSGSGSDYSLTINSLGYDDEGIYHCHQYYEYPLTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 774 - protein, 7 aa, synthetic construct
GYTFTDY

SEQ ID NO: 775 - protein, 10 aa, synthetic construct
GYTFTDYAMH

SEQ ID NO: 776 - protein, 5 aa, synthetic construct
DYAMH

SEQ ID NO: 777 - protein, 6 aa, synthetic construct
TDYAMH

SEQ ID NO: 778 - protein, 8 aa, synthetic construct
GYTFTDYA

SEQ ID NO: 779 - protein, 10 aa, synthetic construct
ASGYTFTDYA

SEQ ID NO: 780 - protein, 6 aa, synthetic construct
IPLIDT

SEQ ID NO: 781 - protein, 10 aa, synthetic construct
SIIPLIDTTS

SEQ ID NO: 782 - protein, 17 aa, synthetic construct
SIIPLIDTTSYNQKFKG

SEQ ID NO: 783 - protein, 13 aa, synthetic construct
WIGSIIPLIDTTS

SEQ ID NO: 784 - protein, 8 aa, synthetic construct
IIPLIDTT

SEQ ID NO: 785 - protein, 17 aa, synthetic construct
IIPLIDTTSYNQKFKGK

SEQ ID NO: 786 - protein, 11 aa, synthetic construct
LGSRYLEGFDY

SEQ ID NO: 787 - protein, 11 aa, synthetic construct
LGSRYLEGFDY

SEQ ID NO: 788 - protein, 11 aa, synthetic construct
LGSRYLEGFDY

SEQ ID NO: 789 - protein, 12 aa, synthetic construct
ARLGSRYLEGFD

SEQ ID NO: 790 - protein, 13 aa, synthetic construct
ARLGSRYLEGFDY

SEQ ID NO: 791 - protein, 10 aa, synthetic construct
LGSRYLEGFD

SEQ ID NO: 792 - protein, 11 aa, synthetic construct
LASQDIGNYLS

SEQ ID NO: 793 - protein, 11 aa, synthetic construct
LASQDIGNYLS

SEQ ID NO: 794 - protein, 11 aa, synthetic construct
LASQDIGNYLS

SEQ ID NO: 795 - protein, 7 aa, synthetic construct
GNYLSWY

SEQ ID NO: 796 - protein, 6 aa, synthetic construct
QDIGNY

SEQ ID NO: 797 - protein, 8 aa, synthetic construct
ASQDIGNY

SEQ ID NO: 798 - protein, 7 aa, synthetic construct
GATNLED

SEQ ID NO: 799 - protein, 7 aa, synthetic construct
GATNLED

SEQ ID NO: 800 - protein, 7 aa, synthetic construct
GATNLED

SEQ ID NO: 801 - protein, 10 aa, synthetic construct
LMIYGATNLE

SEQ ID NO: 802 - skipped

SEQ ID NO: 803 - protein, 12 aa, synthetic construct
GATNLEDGVPSR

SEQ ID NO: 804 - protein, 9 aa, synthetic construct
HQYYEYPLT

SEQ ID NO: 805 - protein, 9 aa, synthetic construct
HQYYEYPLT

SEQ ID NO: 806 - protein, 9 aa, synthetic construct
HQYYEYPLT

SEQ ID NO: 807 - protein, 8 aa, synthetic construct
HQYYEYPL

SEQ ID NO: 808 - protein, 9 aa, synthetic construct
HQYYEYPLT

SEQ ID NO: 809 - protein, 6 aa, synthetic construct
YYEYPL

SEQ ID NO: 810 - protein, 12 aa, synthetic construct
  CROSSLNK 10: note = T is crosslinked to K of SEQ ID NO: 811
ATYYCQQYVTYP

SEQ ID NO: 811 - protein, 24 aa, synthetic construct
  CROSSLNK 16: note = K is crosslinked to T of SEQ ID NO: 810
QRTDSIPNSPDNVPLKGGNCSEDL

SEQ ID NO: 812 - protein, 11 aa, synthetic construct
  CROSSLNK 6: note = S is crosslinked to S of SEQ ID NO: 813
GLEWVSGISWR

SEQ ID NO: 813 - protein, 13 aa, synthetic construct
  CROSSLNK 5: note = S is crosslinked to S of SEQ ID NO: 812
  CROSSLNK 5: note = S is crosslinked to S of SEQ ID NO: 814
GGNCSEDLLCILK

SEQ ID NO: 814 - protein, 16 aa, synthetic construct
  CROSSLNK 7: note = S is crosslinked to S of SEQ ID NO: 813
SLIYAASSLQSGVPSR

SEQ ID NO: 815 - protein, 9 aa, synthetic construct
  CROSSLNK 7: note = Y is crosslinked to S of SEQ ID NO: 816
IGFNWNYEG

SEQ ID NO: 816 - protein, 12 aa, synthetic construct
  CROSSLNK 7: note = S is crosslinked to Y of SEQ ID NO: 815
LKGGNCSEDLLC

SEQ ID NO: 817 - protein, 22 aa, synthetic construct
  CROSSLNK 19: note = H is crosslinked to R of SEQ ID NO: 818
SLRLSCAASGFNFDDYAMHWVR

SEQ ID NO: 818 - protein, 5 aa, synthetic construct
  CROSSLNK 1: note = R is crosslinked to H of SEQ ID NO: 817
RAPFK

SEQ ID NO: 819 - protein, 5 aa, synthetic construct
  CROSSLNK 3: note = Y is crosslinked to N of SEQ ID NO: 820
FDYWG

SEQ ID NO: 820 - protein, 9 aa, synthetic construct
  CROSSLNK 5: note = K is crosslinked to Y of SEQ ID NO: 819
KHLNKTKLS

SEQ ID NO: 821 - protein, 8 aa, synthetic construct
  CROSSLNK 3: note = S is crosslinked to K of SEQ ID NO: 822
LQSGVPSR

SEQ ID NO: 822 - protein, 8 aa, synthetic construct
  CROSSLNK 6: note = K is crosslinked to S of SEQ ID NO: 821
AKHLNKTK

SEQ ID NO: 823 - protein, 13 aa, synthetic construct
  CROSSLNK 5: note = S is crosslinked to R of SEQ ID NO: 824
  CROSSLNK 5: note = S is crosslinked to Y of SEQ ID NO: 824
ISWRSGNIGYADS

SEQ ID NO: 824 - protein, 9 aa, synthetic construct
  CROSSLNK 2: note = R is crosslinked to S of SEQ ID NO: 823
  CROSSLNK 3: note = Y is crosslinked to S of SEQ ID NO: 823
VRYQDGNLV